(19) United States
(12) Patent Application Publication
(10) Pub. No.: US 2006/0100431 A1
(43) Pub. Date: May 11, 2006

(54) Biologically active compounds

(75) Inventors: Martin Quibell, Cambridge (GB); Peter C. Ray, By Kippen (GB); John P. Watts, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Amura Therapeutics Limited, Cambridge (GB)

(21) Appl. No.: 10/521,354

(22) PCT Filed: Jul. 15, 2003
(86) PCT No.: PCT/GB03/02957

§ 371 Date: Oct. 20, 2005

Related U.S. Application Data
(60) Provisional application No. 60/418,524, filed on Oct. 15, 2002.

(30) Foreign Application Priority Data
Jul. 16, 2002 (GB) 0216525.6
Jul. 25, 2002 (GB) 0217239.3

(51) Int. Cl. C07D 487/02

(52) U.S. Cl. 544/333; 546/276.7; 546/272.7; 544/360; 546/271.4; 546/176

(57) ABSTRACT

Compounds of general formula (I) 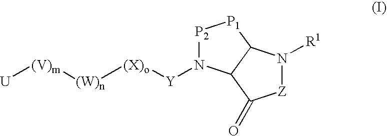
wherein Z=CR³R⁴, where R³ and R⁴ are independently chosen from C₀₋₇-alkyl P₁=CR⁵R⁶, P₂=O, CR⁷R⁸ or NR⁹, Y=CR¹⁰R¹¹—C(O) or CR¹⁰R¹¹—C(S) or CR¹⁰R¹¹—S(O) or CR¹⁰R¹¹—SO₂(X)₀=.CR¹⁶R¹⁷ (W)ₙ=0, S, C(O), S(O) or S(O)₂— or NR¹⁸(V)ₘ=C(O), C(S), S(O), S(O)₂, S(O)₂NH, OC(O), NHC(O), NHS(O), NHS(O)₂, OC(O)NH, C(O)NH or CR¹⁹R²⁰, C═N—C(O)—OR¹⁹ or C═N═C(O)—NHR¹⁹, U=a stable. 5- to 7 membered monocyclic or a stable 8- to 11-membered bicyclic ring which is either saturated or unsaturated, and which includes zero to four heteroatoms and their sales, hydrates, solvates, complexes and prodruges are inhibitors of cathepsin K and other cysteine protease inhibitors and are useful as therapeutic agents, for example in osteoporosis, Paget's disease gingival diseases such as gingivitis and periodontis, hypercalaemia of malignancy, metabolic bone disease, diseases involving matrix or cartilage degradation, in particular osteoarthitis and rheumatoid arthritis and neoplastic diseases. The compounds are also useful for validating therapeutic target compounds.

THIS INVENTION relates to compounds which are inhbitors across a broad range of cysteine proteases, to the use of these compounds, and to pharmaceutical compositions comprising them. Particular compounds of the invention are inhibitors of cathepsin K and related cysteine protesases of the CA clan. Furthermore, such compounds are useful for the in vivo therapeutic treatment of diseases in which participation of a cysteine protease is implicated.

Proteases form a substantial group of biological molecules which to date constitute approximately 2% of all the gene products identified following analysis of several completed genome sequencing programmes. Proteases have evolved to participate in an enormous range of biological processes, mediating their effect by cleavage of peptide amide bonds within the myriad of proteins found in nature. This hydrolytic action is performed by initially recognising, then binding to, particular three-dimensional electronic surfaces displayed by a protein, which aligns the bond for cleavage precisely within the protease catalytic site. Catalytic hydrolysis then commences through nucleophilic attack of the amide bond to be cleaved either via an amino acid side-chain of the protease itself, or through the action of a water molecule that is bound to and activated by the protease. Proteases in which the attacking nucleophile is the thiol side-chain of a Cys residue are known as cysteine proteases. The general classification of ‘cysteine protease’ contains many members found across a wide range of organisms from viruses, bacteria, protozoa, plants and fungi to mammals.

Cathepsin K and indeed many other crucial proteases belong to the papain-like CA C1 family. Cysteine proteases are classified into ‘clans’ based upon a similarity in the three-dimensional structure or a conserved arrangement of catalytic residues within the protease primary sequence. Additionally, ‘clans’ may be further classified into ‘families’ in which each protease shares a statistically significant relationship with other members when comparing the portions of amino acid sequence which constitute the parts responsible for the protease activity (see Barrett, A. J et al, in ‘Handbook of Proteolytic Enzymes’, Eds.

Barrett, A. J., Rawlings, N. D., and Woessner, J. F. Publ. Academic Press, 1998, for a thorough discussion).

To date, cysteine proteases have been classified into five clans, CA, CB, CC, CD and CE (Barrett, A. J. et al, 1998). A protease from the tropical papaya fruit ‘papain’ forms the foundation of clan CA, which currently contains over 80 distinct and complete entries in various sequence databases, with many more expected from the current genome sequencing efforts. Proteases of clan CA/family C1 have been implicated in a multitude of house-keeping roles and disease processes. e.g. human proteases such as cathepsin K (osteoporosis), cathepsin S (autoimmune disorders), cathepsin L (metastases), cathepsin B (metastases, arthritis), cathepsin F (antigen processing), cathepsin V (T-cell selection), dipeptidyl peptidase I (granulocyte serine protease activation) or parasitic proteases such as falcipain (malaria parasite Plasmodium falciparum) and cruzipain (Trypanosoma cruzi infection). Recently a bacterial protease, staphylopain (S. aureus infection) has also been tentatively assigned to clan CA. X-ray crystallographic structures are available for a range of the above mentioned proteases in complex with a range of inhibitors e.g. papain (PDB entries, 1pad, 1pe6, 1pip, 1pop, 4pad, 5pad, 6pad, 1ppp, 1the, 1csb, 1huc), cathepsin K (1au0, 1au2, 1au3, 1au4, 1atk, 1mem, 1bgo, 1ayw, 1ayu, 1 nl6, 1nlj), cathepsin L (1cs8, 1mhw), cathepsin S (1glo, 1ms6 and currently on-hold but published McGrath, M. E. et al, Protein Science, 7, 1294-1302, 1998), cathepsin V (1fh0), dipeptidyl peptidase I (1jqp, 1k3b), cathepsin B (1gmy), cathepsin F (currently on-hold, but published Somoza, J. R. et al, J. Mol. Biol., 322, 559-568, 2002), cruzain (a recombinant form of cruzipain see Eakin, A. E. et al, 268(9), 6115-6118, 1993) (1ewp, 1aim, 2aim, 1F29, 1F2A, 1F2B, 1F2C), staphylopain (1cv8). Each of the structures displays a similar overall active-site topology, as would be expected by their ‘clan’ and ‘family’ classification and such structural similarity exemplifies one aspect of the difficulties involved in discovering a selective inhibitor of cathepsin K suitable for human use. However, subtle differences in terms of the depth and intricate shape of the active site groove of each CA C1 protease are evident, which may be exploited for selective inhibitor design. Additionally, many of the current substrate-based inhibitor complexes of CA C1 family proteases show a series of conserved hydrogen bonds between the inhibitor and the protease backbone, which contribute significantly to inhibitor potency. Primarily a bidentate hydrogen-bond is observed between the protease Gly66 (C═O)/inhibitor N—H and the protease Gly66(NH)/inhibitor (C═O), where the inhibitor (C═O) and (NH) are provided by an amino acid residue NHCHRCO that constitutes the S2 sub-site binding element within the inhibitor (see Berger, A. and Schecter, I. Philos. Trans. R. Soc. Lond. [Biol.], 257, 249-264, 1970 for a description of protease binding site nomenclature). A further hydrogen-bond between the protease main-chain (C═O) of asparagine or aspartic acid (158 to 163, residue number varies between proteases) and an inhibitor (N—H) is often observed, where the inhibitor (N—H) is provided by the S1 sub-site binding element within the inhibitor. Thus, the motif X—NHCHRCO—NH—Y is widely observed amongst the prior art substrate-based inhibitors of CA C1 proteases.

Cathepsin K is thought to be significant in diseases involving excessive loss of bone or cartilage. Bone consists of a protein matrix incorporating hydroxyapatite crystals. About 90% of the structural protein of the matrix is type I collagen, with the remainder comprising various non-collagenous proteins such as osteocalcin, proteoglycans, osteopontin, osteonectin, thrombospondin, fibronectin and bone sialoprotein.

Skeletal bone is not a static structure but continually undergoes a cycle of bone resorption and replacement. Bone resorption is carried out by osteoclasts, which are multinuclear cells of haematopoietic lineage. Osteoclasts adhere to the bone surface and form a tight sealing zone. The membrane on the apical surface of the osteoclasts is folded so as to create a closed extracellular compartment between the osteoclast and the bone surface, which is acidified by proton pumps in the osteoclast membrane. Proteolytic enzymes are secreted into the compartment from the osteoclast. The high acidity in the compartment causes the hydroxyapatite at the surface of the bone to be dissolved and the proteolytic enzymes break down the protein matrix causing a resorption lacuna to be formed. Following bone resorption, osteoblasts produce a new protein matrix that is subsequently mineralised.

In disease states such as osteoporosis and Paget's disease, the bone resorption and replacement cycle is disrupted leading to a net loss of bone with each cycle. This leads to weakening of the bone and therefore to increased risk of bone fracture.

Cathepsin K is expressed at a high level in osteoclasts and is therefore thought to be essential for bone resorption. Therefore, selective inhibition of cathepsin K is likely to be effective in the treatment of diseases involving excessive bone loss. These include osteoporosis, gingival diseases such as gingivitis and periodontitis, Paget's disease, hypercalaemia of malignancy and metabolic bone disease.

In addition to osteoclasts, high levels of cathepsin K are also found in chondroclasts from the synovium of osteoarthritic patients. It therefore appears that cathepsin K inhibitors will be of use in the treatment of diseases involving matrix or cartilage degradation, in particular osteoarthritis and rheumatoid arthritis.

Elevated levels of cathepsin K are also found in metastatic neoplastic cells which suggests that cathepsin K inhibitors may also be useful for treating certain neoplastic diseases.

In the prior art, the development of cysteine protease inhibitors for human use has recently been an area of intense activity (e.g. see Bromme, D. and Kaleta, J., Curr. Pharm. Des., 8, 1639-1658, 2002; Kim, W. and Kang, K., Expert Opin. Ther. Patents, 12(3), 419-432, 2002; Leung-Toung, R. et al. Curr. Med. Chem., 9, 979-1002, 2002; Lecaille, F. et al., Chem. Rev., 102, 4459-4488, 2002; Hernandez, A. A. and Roush, W. R., Curr. Opin. Chem. Biol., 6, 459-465, 2002). Considering the CA C1 family members, particular emphasis has been placed upon the development of inhibitors of human cathepsins, primarily cathepsin K (osteoporosis), cathepsin S (autoimmune disorders), cathepsin L (metastases), cathepsin B (metastases, arthritis), cathepsin F (antigen processing), cathepsin V (T-cell selection) and dipeptidyl peptidase I (granulocyte serine protease activation), through the use of peptide and peptidomimetic nitriles (e.g. see WO-A-03041649, WO-A-03037892, WO-A-03029200, WO-A-02051983, WO-A-02020485, US-A-20020086996, WO-A-01096285, WO-A-0109910, WO-A-0051998, WO-A-0119816, WO-A-9924460, WO-A-0049008, WO-A-0048992, WO-A-0049007, WO-A-0130772, WO-A-0055125, WO-A-0055126, WO-A-0119808, WO-A-0149288, WO-A-0147886), linear and cyclic peptide and peptidomimetic ketones (e.g. see Veber, D. F. and Thompson, S. K., Curr. Opin. Drug Discovery Dev., 3(4), 362-369, 2000, WO-A-02092563, WO-A-02017924, WO-A-01095911, WO-A-0170232, WO-A-0178734, WO-A-0009653, WO-A-0069855, WO-A-0029408, WO-A-0134153 to WO-A-0134160, WO-A-0029408, WO-A-9964399, WO-A-9805336, WO-A-9850533), ketoheterocycles (e.g. see WO-A-02080920, WO-A-03042197, WO-A- WO-A-03024924, WO-A-0055144, WO-A-0055124), monobactams (e.g. see WO-A-0059881, WO-A-9948911, WO-A-0109169), α-ketoamides (e.g. see WO-A-03013518), cyanoamides (WO-A-01077073, WO-A-01068645), dihydro pyrimidines (e.g. see WO-A-02032879) and cyanoaminopyrimidines (e.g. see WO-A-03020278, WO-A-03020721). The prior art describes potent in vitro inhibitors, but also highlights the many difficulties in developing a human therapeutic. For example, WO-A-9850533 and WO-A-0029408 describe compounds that may be referred to as cyclic ketones and are inhibitors of cysteine proteases with a particular reference towards papain family proteases and as a most preferred embodiment, cathepsin K. WO-A-9850533 describes compounds subsequently detailed in the literature as potent inhibitors of cathepsin K with good oral bioavailability (Witherington, J., ‘Tetrahydrofurans as Selective Cathepsin K Inhibitors’, RSC meeting, Burlington House, London, 1999). The compounds of WO-A-9850533 were reported to bind to cathepsin K through the formation of a reversible covalent bond between the tetrahydrofuran carbonyl and the active site catalytic cysteine residue (Witherington, J., 1999). Additionally, the same cyclic ketone compounds are described in WO-A-9953039 as part of a wide-ranging description of inhibitors of cysteine proteases associated with parasitic diseases, with particular reference to the treatment of malaria by inhibition of falcipain. However, subsequent literature describes the cyclic ketone compounds of WO-A-9850533 to be unsuitable for further development or for full pharmacokinetic evaluation due to a physiochemical property of the inhibitors, the poor chiral stability of the α-aminoketone chiral centre (Marquis, R. W. et al, J. Med. Chem., 44(5), 725-736, 2001). WO-A-0069855 describes compounds that may also be referred to as cyclic ketones with particular reference towards inhibition of cathepsin S. The compounds of WO-A-0069855 are considered to be an advance on compounds of WO-A-9850533 due to the presence of the β-substituent on the cyclic ketone ring system that provides chiral stability to the α-carbon of the cyclic ketone ring system. However, the compounds of WO-A-0069855 and indeed those of WO-A-9850533 describe a requirement for the presence of the potential hydrogen-bonding motif X—NHCHRCO—NH—Y that is widely observed amongst the prior art substrate-based inhibitors of CA C1 proteases.

Our earlier patent application (WO-A-02057270) describes bicyclic compounds in which the chirality of the α-aminoketone is stabilised (for a review of energetic considerations within fused ring systems see Toromanoff, E. Tetrahedron Report No 96, 36, 2809-2931, 1980). These compounds do not contain the X—NHCHRCO—NH—Y motif and yet the compounds are highly potent inhibitors across a broad range of CA C1 cysteine proteases. In particular, certain of the compounds are potent and selective cruzipain inhibitors.

The present invention relates to variants of the compounds described in WO-A-02057270 which are also inhibitors of a wide range of CA C1 cysteine protease. In particular, some compounds of the present invention are potent and selective inhibitors of cathepsin K

Therefore, in the present invention, there is provided a compound of general formula (I) 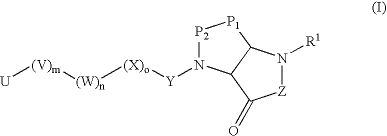
wherein:
Z=CR³R⁴, where R³ and R⁴ are independently chosen from C₀₋₇-alkyl (when C=0, R³ or R⁴ is simply a hydrogen atom), C₃₋₆-cycloalkyl, Ar—C₀₋₄-alkyl (when C=0, R³ or R⁴ is simply an aromatic moiety Ar),
P₁=CR⁵R⁶, where R⁵ and R⁶ are independently chosen from C₀₋₇-alkyl, C₃₋₆-cycloalkyl, Ar—C₀₋₇-alkyl, O—C₀₋₇-alkyl, O—C₃₋₆-cycloalkyl, O—Ar—C₀₋₇-alkyl, S—C₀₋₇-alkyl, S—C₃₋₆-cycloalkyl, S—Ar—C₀₋₇-alkyl, NH—C₀₋₇-alkyl, NH—C₃₋₆-cycloalkyl, NH—Ar—C₀₋₇-alkyl, N(C₀₋₇-alkyl)₂, N(C₃₋₆-cycloalkyl)₂ or N(Ar—C₀₋₇-alkyl)₂;
P₂=O, CR⁷R⁸ or NR⁹, where R⁷ and R⁸ are independently chosen from C₀₋₇-alkyl, C₃₋₆-cycloalkyl, Ar—C₀₋₇-alkyl and R⁹ is chosen from C₀₋₇-alkyl, C₃₋₆-cycloalkyl or Ar—C₀₋₇-alkyl;
Y=CR¹⁰R¹¹—C(O) or CR¹⁰R¹¹—C(S) or CR¹⁰R¹¹—S(O) or CR¹⁰R¹¹—SO₂ where R¹⁰ and R¹¹ are independently chosen from C₀₋₇-alkyl, C₃₋₆-cycloalkyl and Ar—C₀₋₇-alkyl, or Y represents 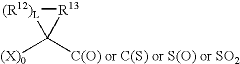
where L is a number from one to four and R¹² and R¹³ are independently chosen from CR¹⁴R¹⁵ where R¹⁴ and R¹⁵ are independently chosen from C₀₋₇-alkyl, C₃₋₆-cycloalkyl, Ar—C₀₋₇-alkyl or halogen; and for each R¹² and R¹³ either R¹⁴ or R¹⁵ (but not both R¹⁴ and R¹⁵) may additionally be chosen from O—C₀₋₇-alkyl, O—C₃₋₆-cycloalkyl, O—Ar—C₀₋₇-alkyl, S—C₀₋₇-alkyl, S—C₃₋₆-cycloalkyl, S—Ar—C₀₋₇-alkyl, NH—C₀₋₇-alkyl, NH—C₃₋₆-cycloalkyl, NH—Ar—C₀₋₇-alkyl, N—(C₀₋₇-alkyl)₂, N—(C₃₋₆-cycloalkyl)₂, and N—(Ar—C₀₋₇-alkyl)₂;
(X)ₒ=CR¹⁶R¹⁷, where R¹⁶ and R¹⁷ are independently chosen from C₀₋₇-alkyl, C₃₋₆-Cycloalkyl and Ar—C₀₋₇-alkyl and o is a number from zero to three;
(W)ₙ=O, S, C(O), S(O) or S(O)₂ or NR¹⁸, where R¹⁸ is chosen from C₀₋₇-alkyl, C₃₋₆-cycloalkyl and Ar—C₀₋₇-alkyl and n is zero or one;
(V)ₘ=C(O), C(S), S(O), S(O)₂, S(O)₂NH, OC(O), NHC(O), NHS(O), NHS(O)₂, OC(O)NH, C(O)NH or CR¹⁹R²⁰, C═N—C(O)—OR¹⁹ or C═N—C(O)—NHR¹⁹, where R¹⁹ and R²⁰ are independently chosen from C₀₋₇ alkyl, C₃₋₆-cycloalkyl, Ar—C₀₋₇-alkyl and m is a number from zero to three, provided that when m is greater than one, (V)ₘ contains a maximum of one carbonyl or sulphonyl group;
U=a stable 5- to 7-membered monocyclic or a stable 8- to 11-membered bicyclic ring which is either saturated or unsaturated and which includes zero to four heteroatoms (as detailed below): 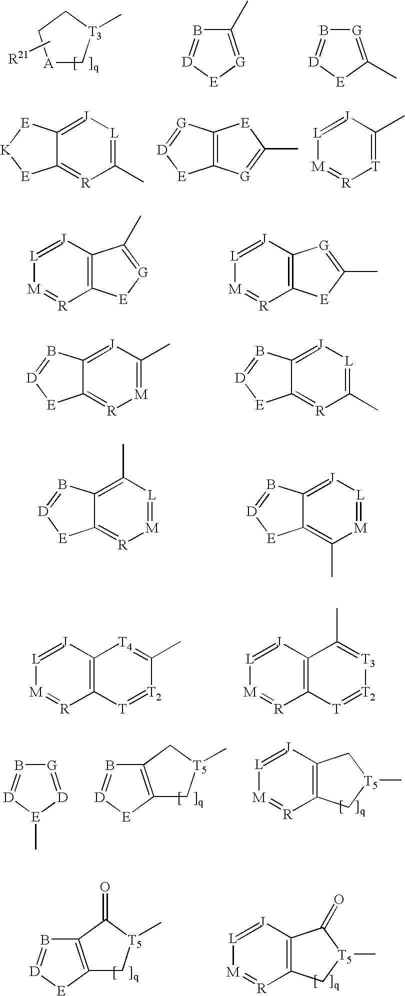 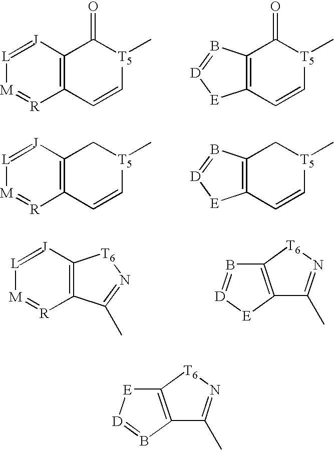
wherein R²¹ is:
   C₀₋₇-alkyl, C₃₋₆-cycloalkyl, Ar—C₀₋₇-alkyl, O—C₀₋₇-alkyl, O—C₃₋₆-cycloalkyl, —O—Ar—C₀₋₇-alkyl, S—C₀₋₇-alkyl, S—C₃₋₆-cycloalkyl, S—Ar—C₀₋₇-alkyl, SO₂—C₀₋₇-alkyl, SO₂—C₃₋₆-cycloalkyl, SO₂—Ar—C₀₋₇-alkyl, NH—C₀₋₇-alkyl, NH—C₃₋₆-cycloalkyl, NH—Ar—C₀₋₇-alkyl, N(C₀₋₇-alkyl)₂, N(C₃₋₆-cycloalkyl)₂ or N(Ar—C₀₋₇-alkyl)₂; or, when part of a CHR²¹ or CR²¹ group, R²¹ may be halogen;
A is chosen from:
   CH₂, CHR²¹, O, S, SO₂, NR²² or N-oxide (N→O), where R²¹ is as defined above; and R²² is chosen from C₀₋₇-alkyl, C₃₋₆-cycloalkyl and Ar—C₀₋₇-alkyl;
B, D and G are independently chosen from:
   CR²¹, where R²¹ is as defined above, or N or N-oxide (N→O);
E is chosen from:

CH₂, CHR²¹, O, S, SO₂, NR² or N-oxide (N→O), where R²¹ and R²² are defined as above;
K is chosen from:
   CH₂, CHR²², where R²² is defined as above;
J, L, M, R, T, T₂, T₃ and T₄ are independently chosen from:
   CR²¹ where R²¹ is as defined above, or N or N-oxide (N→O);
T₅ is chosen from:
   CH or N;
T₆ is chosen from:
   NR²², SO₂, OC(O), C(O), NR²²C(O);
q is a number from one to three, thereby defining a 5-, 6- or 7-membered ring;
R¹═R²C(O), R²OC(O), R²NQC(O), R²SO₂, where R² is chosen from C₁₋₇-alkyl, C₃₋₆-cycloalkyl or Ar—C₀₋₇-alkyl (when C=0, R² is simply an aromatic moiety Ar) and Q is C₀₋₇-alkyl;
provided that when Y is other than CR¹⁰R¹¹—C(O) or when U is: 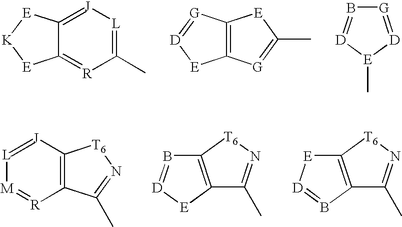
R¹ may also be C₀₋₇-alkyl, C₃₋₆-cycloalkyl or Ar—C₇-alkyl.

The present invention includes all salts, hydrates, solvates, complexes and prodrugs of the compounds of this invention. The term “compound” is intended to include all such salts, hydrates, solvates, complexes and prodrugs, unless the context requires otherwise.

Appropriate pharmaceutically and veterinarily acceptable salts of the compounds of general formula (I) include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Salts which are not pharmaceutically or veterinarily acceptable may still be valuable as intermediates.

Prodrugs are any covalently bonded compounds which release the active parent drug according to general formula (I) in vivo. A prodrug may for example constitute an acetal or hemiacetal derivative of the exocyclic ketone functionality present in the hexahydropyrrolo[3,2-b]pyrrol-3-one, hexahydropyrrolo[3,2-c]pyrazol-6-one or hexahydro-2-oxa-1,4-diazapentalen-6-one scaffold. If a chiral centre or another form of isomeric centre is present in a compound of the present invention, all forms of such isomer or isomers, including enantiomers and diastereoisomers, are intended to be covered herein. Compounds of the invention containing a chiral centre may be used as a racemic mixture, an enantiomerically enriched mixture, or the racemic mixture may be separated using well-known techniques and an individual enantiomer may be used alone.

‘Halogen’ as applied herein is meant to include F, Cl, Br, I;

‘Heteroatom’ as applied herein is meant to include O, S and N;

‘C₀₋₇-alkyl’ as applied herein is meant to include stable straight and branched chain aliphatic carbon chains containing zero (i.e. simply hydrogen) to seven carbon atoms such as methyl, ethyl, n-propyl, isopropyl, n-butyl, isobutyl, t-butyl, pentyl, isopentyl, hexyl, heptyl and any simple isomers thereof. Additionally, where ‘C₀₋₇-alkyl’ contains 2 or more contiguous carbon atoms, an alkene (—CH═CH—) may be present. Additionally, any C₀₋₇-alkyl may optionally be substituted at any point by one, two or three halogen atoms (as defined above) for example to give a trifluoromethyl substituent. Furthermore, C₀₋₇-alkyl may contain one or more heteroatoms (as defined above) for example to give ethers, thioethers, sulphones, sulphonamides, substituted amines, amidines, guanidines, carboxylic acids, carboxamides. If the heteroatom is located at a chain terminus then it is appropriately substituted with one or two hydrogen atoms. A heteroatom or halogen is only present when C₀₋₇-alkyl contains a minimum of one carbon atom. For example, the group CH₃—CH₂—O—CH₂—CH₂— is defined within ‘C₀₋₇-alkyl’ as a C₄ alkyl that contains a centrally positioned heteroatom whereas the group CH₃—CH₂—CH₂—CH₂— is defined within ‘C₀₋₇-alkyl’ as an unsubstituted C₄ alkyl.

‘C₃₋₆-cycloalkyl’ as applied herein is meant to include any variation of ‘C₀₋₇-alkyl’ which additionally contains a carbocyclic ring such as cyclopropyl, cyclobutyl, cyclopentyl, cyclohexyl. The carbocyclic ring may optionally be substituted at any position with one or more halogens (as defined above) or heteroatoms (as defined above) for example to give a tetrahydrofuran, pyrrolidine, piperidine, piperazine or morpholine substituent.

‘Ar—C₀₋₇-alkyl’ as applied herein is meant to include any variation of C₀₋₇-alkyl which additionally contains an aromatic ring moiety ‘Ar’. The aromatic ring moiety Ar can be a stable 5 or 6-membered monocyclic or a stable 8 to 10 membered bicyclic ring which is unsaturated, as defined previously for U in general formula (I). The aromatic ring moiety Ar may be substituted by R²¹ (as defined above for U in general formula (I)). When C=0 in the substituent Ar—C₀₋₇-alkyl, the substituent is simply the aromatic ring moiety Ar.

Other expressions containing terms such as alkyl and cycloalkyl are intended to be construed according to the definitions above. For example “C₁₋₄ alkyl” is the same as C₀₋₇-alkyl except that it contains from one to four carbon atoms.

If different structural isomers are present, and/or one or more chiral centres are present, all isomeric forms are intended to be covered. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985). It is also intended to include compounds of general formula (I) where any hydrogen atom has been replaced by a deuterium atom.

Compounds of general formula I are inhibitors of a wide range of CA C1 cysteinyl proteases for example cathepsin K, cathepsin S, cathepsin L, cathepsin F, cathepsin B, cruzipains, falcipains and leismania mexicana CPB protease.

For all the above mentioned CA C1 proteases, the preferred fundamental backbone shape of inhibitor molecules is broadly similar. Therefore, the preferred compounds of general formula (I) will have similar (V)ₘ, (W)ₙ, (X)ₒ and R¹ whether they act as cathepsin K cathepsin S, cathepsin L, cathepsin F, cathepsin B, cruzipains, falcipains or leismania mexicana CPB protease inhibitors. Within general formula (I), inhibitory potency and selectivity for each CA C1 protease is primarily determined by different preferences for the Y and U groups for each CA C1 protease.

Preferred compounds of general formula (I) include, but are not limited to those which, independently or in any combination:

Z is CH₂;

P¹ is CH₂;

P² is CH₂, O or NH.

As mentioned above, cysteine protease inhibitors of general formula (I), comprise an R² group chosen from C₁₋₇-alkyl, C₃-cycloalkyl and Ar—C₀₋₇-alkyl.

When R² comprises Ar—C₀₋₇-alkyl, preferred R² groups comprise Ar—C₀₋₂-alkyl and examples include but are not limited to: 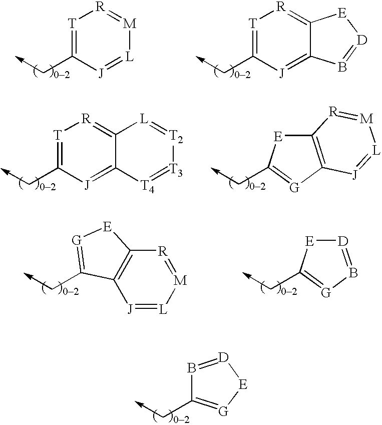
where J, L, M, R, T, T₂, T₃ and T₄, B, D, G and E are as previously defined.

More preferred R² comprises Ar—C₀₋₁-alkyl and examples of such R² groups include, but are not limited to: 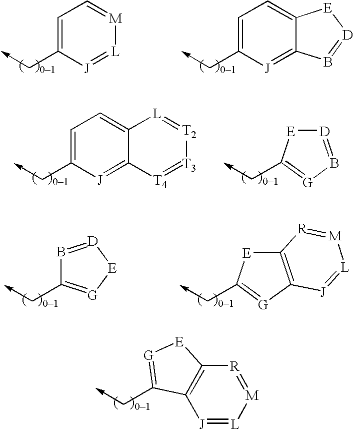
where J, L, M, T₂, T₃, T₄, B, D, G and E are as previously defined.

Still more active compounds of general formula (I) are those in which R² comprises a monocyclic Ar—C₀₋₁-alkyl and forms part of an R¹ group such as: 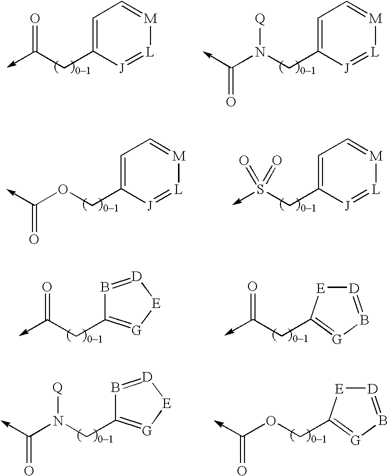
wherein:
J, L, M, B, D and G are as defined above (i.e. CR²¹, N or N→O) and wherein R²¹ is chosen from hydrogen, methyl, methoxy, ethyl, isopropyl, trifluoromethyl, trifluoromethoxy, F, Cl, SO₂Me; and

E is as previously defined; and

Q is chosen from hydrogen or methyl.

In cysteine protease inhibitors of general formula (I) when R² is C₁₋₇-alkyl, preferred R² groups comprise C₃₋₇-alkyl which may include an —O— or —NH— as part of the chain and which is either unsubstituted or is substituted with one or more NH₂, NHMe, NHC(O)CH₃, NMeC(O)CH₃, OH or OMe groups.

When R² is C₃₋₇-alkyl, more preferred groups include C₃₋₆-alkyl, in particular those which are branched at the α-position or which include an NH₂, NHMe, NHC(O)CH₃, NMeC(O)CH₃, OH or OMe substituent at the α-position.

In cysteine protease inhibitors of general formula (I) when R² is C₃₋₆-cycloalkyl, R² may include a heteroatom in the ring system. Examples of R² groups include cyclopropyl, cyclobutyl, cyclopentyl, cyclohexyl, pyrrolidine, piperidine, morpholine, tetrahydrofuran, cyclopentene, cyclopentadiene, cyclohexadiene and piperazine. Nitrogen-containing rings may be N-substituted with groups such as C₁₋₄ alkyl, phenyl or benzyl.

It is yet more preferred that when R² is a C₃₋₆-cycloalkyl group, the ring system is either connected directly to the remainder of the R¹ moiety or there is one intervening methylene group. The inventors have found that the activity of the molecule increases with the size of the cycloalkyl ring and therefore compounds in which R² is a five- or six-membered cyclic ring are most favourable.

In compounds of general formula (I), particularly preferred R¹ groups therefore include: benzoyl; pyridine-2-carbonyl; 1-oxy-pyridine-2-carbonyl; pyridine-3-carbonyl; 1-oxy-pyridine-3-carbonyl; pyridine-4-carbonyl; 1-oxy-pyridine-4-carbonyl; phenyl sulphonyl; pyridine-2-sulphonyl; 1-oxy-pyridine-2-sulphonyl; pyridine-3-sulphonyl; 1-oxy-pyridine-3-sulphonyl; pyridine-4-sulphonyl; 1-oxy-pyridine-4 sulphonyl; phenylacetyl; phenylcarbamoyl; isobutylcarbamoyl; phenyloxycarbonyl; isobutyloxycarbonyl; pyrrolidine-N-carbonyl; piperidine-N-carbonyl; morpholin-N-carbonyl; piperazine-N-carbonyl; 4-methyl-piperazie-N-carbonyl; (4-methyl-piperazin-1-yl)-acetoyl; piperazin-1-yl-acetoyl; furan-2-carbonyl; 5-chlorofuran-2-carbonyl; thiophene-2-carbonyl; 5-chlorothiophene-2-carbonyl; furan-3-carbonyl; thiophene-3-carbonyl; cyclopentoyl; cyclohexoyl; cyclopent-3-enoyl; cyclopentylmethylcarbonyl; cyclohexylmethylcarbonyl; pyrrolidine-2-carbonyl; N-acetyl-pyrrolidine-2-carbonyl; piperidine-2-carbonyl; N-acetyl-piperidine-2-carbonyl; tetrahydrofuran-2-carbonyl; 1-aminocyclobutanoyl; 1-aminocyclopentanoyl; 1-aminocyclohexanoyl; N-acetyl-1-aminocyclobutanoyl; N-acetyl-1-aminocyclopentanoyl; N-acetyl-1-aminocyclohexanoyl; 1-hydroxycyclobutanoyl; 1-hydroxycyclopentanoyl; 1-hydroxycyclohexanoyl; 1-methoxycyclobutanoyl; 1-methoxycyclopentanoyl; 1-methoxycyclohexanoyl; aminocyclopentylacetoyl; aminocyclohexylacetoyl; N-acetylaminocyclopentylacetoyl; N-acetylaminocyclohexylacetoyl; 2-acetylaminopropionoyl; 2-acetylaminoethanoyl; 2-acetyl-N-methylaminoethanoyl; N,N-dimethylaminoacetoyl; 2-aminobutanoyl; N-acetyl-2-aminobutanoyl; 2-amino-3-methylbutanoyl; N-acetyl-2-amino-3-methylbutanoyl, 2-amino-3,3-dimethylbutanoyl; N-acetyl-2-amino-3,3-dimethylbutanoyl; 2-amino-3-methylpentanoyl; N-acetyl-2-amino-3-methylpentanoyl; pentanoyl; 3-methylpentanoyl; 4-methylpentanoyl; 2-amino-4-methylpentanoyl; N-acetyl-2-amino-4-methylpentanoyl; 2-amino-4,4-dimethylpentanoyl; N-acetyl-2-amino-4,4-dimethylpentanoyl; 2-aminopentanoyl; N-acetyl-2-aminopentanoyl; 2-amino-5-methylhexanoyl; N-acetyl-2-amino-5-methylhexanoyl; 2-hydroxy-3-methylbutanoyl; 2-methoxy-3-methylbutanoyl; 2-hydroxy-3,3-dimethylbutanoyl; 2-methoxy-3,3-dimethylbutanoyl; 2-hydroxy-3-methylpentanoyl; 2-methoxy-3-methylpentanoyl; 2-hydroxy-4-methylpentanoyl; 2-methoxy-methylpentanoyl; 2-hydroxy-4,4-dimethylpentanoyl; 2-methoxy-4,4-dimethylpentanoyl; 2hydroxypentanoyl; 2-methoxypentanoyl; 2-hydroxy-5-methylhexanoyl; 2-methoxy-5-methylhexanoyl;

In cysteine protease inhibitors of general formula (I), it is preferred that in the group (X)ₒ, each of R¹⁶ and R¹⁷ is selected from C₀₋₇-alkyl or Ar—C₀₋₇-alkyl, for example hydrogen, a straight or branched alkyl chain, a straight or branched heteroalkyl chain, an optionally substituted arylalkyl chain or an optionally substituted arylheteroalkyl chain.

More preferred (X)ₒ groups comprise R₁₆ chosen from hydrogen; R¹⁷ chosen from hydrogen or C₁₋₄-alkyl, which may be substituted with OH, NR²²R²², COOR²², or CONR²²; or Ar—C₁₋₄-alkyl, where the aryl group may be substituted with R²¹, wherein each R²¹ and R²² is independently as defined previously.

Yet more preferred (X)ₒ groups are those in which R¹⁶ is from hydrogen and R¹⁷ is chosen from hydrogen or simple C₁₋₄-alkyl groups such as methyl, ethyl, propyl, butyl.

In the most preferred (X)ₒ groups, R¹⁶ and R¹⁷ are hydrogen and o is zero or one.

Preferred compounds of general formula (I) are those in which, in the group (W)ₙ, W is chosen from O, S, SO₂, S(O), C(O) or NR¹⁸, where R¹⁸ is chosen from C₀₋₇-alkyl; and n is zero or one.

In more preferred (W)ₙ groups, W comprises O, S, SO₂, C(O) or NH where n is zero or one.

Still more active compounds are those in which W is C(O) or NH where n is zero or one.

It is most preferred that in the group (W)ₙ, W is NH and n is zero or one.

In protease inhibitors of general formula (I), more active compounds are those in which, in the group (V)ₘ, V is chosen from C(O), OC(O), NHC(O), C(O)NH, CHR²⁰; C—N—C(O)—OR¹⁹ or C═N—C(O)—NHR¹⁹
where R¹⁹ is chosen from C₀₋₇-alkyl, C₃₋₆-cycloalkyl, Ar—C₀₋₇-alkyl and R²⁰ is C₀₋₄-alkyl, and
   m is zero or one.

Examples of preferred V and W substituent combinations include, but are not limited to:

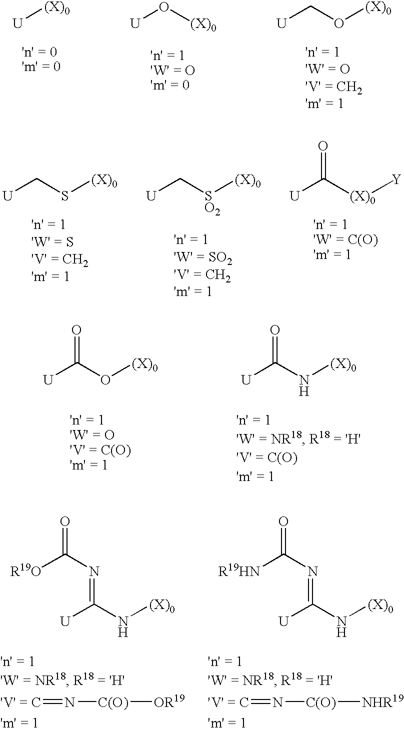

Preferred V, W and X substituent combinations include, but are not limited to:

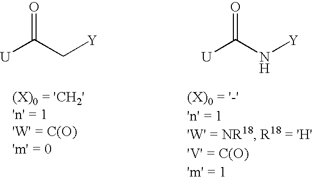

As mentioned above, the substituents Y and U are important in determining the inhibitory potency and selectivity for various proteases and the preferred Y and U substituents vary depending on the target protease.

In compounds of general formula (I) that are inhibitors of cathepsin K, it is preferred that the Y substituent is CHR¹¹CO where R¹¹ is selected from C₀₋₇-alkyl, Ar—C₀₋₇-alkyl or C₃₋₆-cycloalkyl. Examples of suitable R¹¹ groups include, for example, hydrogen, a straight or branched alkyl chain, a straight or branched heteroalkyl chain, an optionally substituted arylalkyl chain or an optionally substituted arylheteroalkyl chain, cyclohexylmethyl or cyclopentylmethyl. Additionally, preferred compounds of general formula (I) are those in which Y comprises a group: 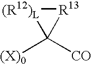
where R¹² and R¹³ are each CR¹⁴R¹⁵ and each R¹⁴ and R¹⁵ is, independently, selected from C₀₋₇-alkyl or Ar—C₀₋₇-alkyl, for example hydrogen, a straight or branched alkyl chain, a straight or branched heteroalkyl chain, an optionally substituted arylalkyl chain or an optionally substituted arylheteroalkyl chain and L is a number from one to four.

Examples of preferred Y substituents in compounds of general formula (I) which are inhibitors of cathepsin K include, but are not limited to: 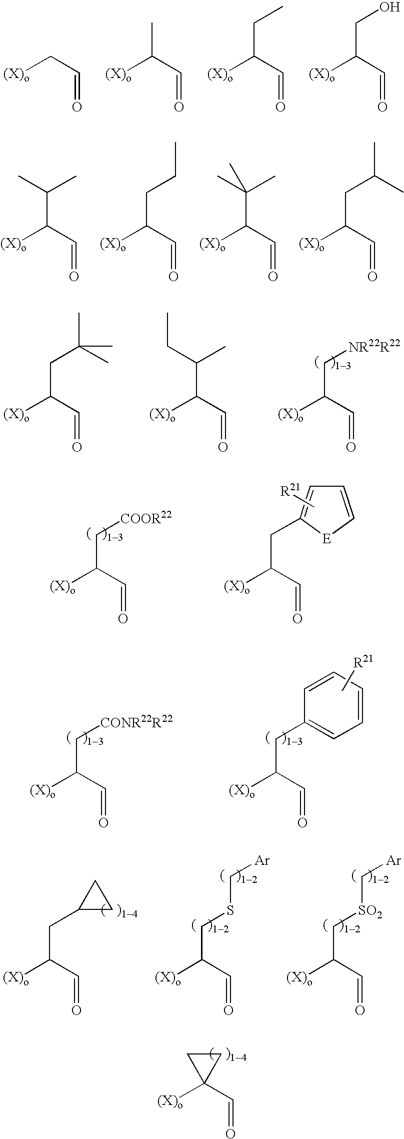
wherein E, R²¹, R²² and Ar are as defined previously, any of which may be substituted with one or more halogen, preferably fluoro, substituents.

In compounds that are inhibitors of cathepsin K, more preferred R¹¹ groups include C₁₋₄-alkyl, which may be substituted with cycloalkylmethyl or halogen, or R¹¹ is chosen from cycloalkyl-1-carbonyl or R¹¹ is chosen from Ar—C₁₋₄-alkyl, where the aryl group may be substituted with R²¹; where R²¹ is defined above.

Increased inhibition of cathepsin K can be achieved in compounds in which the R¹¹ groups are simple branched alkyl groups such as isobutyl or straight alkyl chains such as n-propyl, optionally substituted with one or more halogen (preferably fluoro) substitutents. Yet more preferred R¹¹ groups comprise ArCH₂—, where the aromatic ring is an optionally substituted monocyclic heterocycle and still more preferred R¹¹ groups comprise cyclopropylmethyl and cyclohexyl-1-carbonyl. In compounds which are particularly active inhibitors of cathepsin K, Y substituents include, but are not limited to: 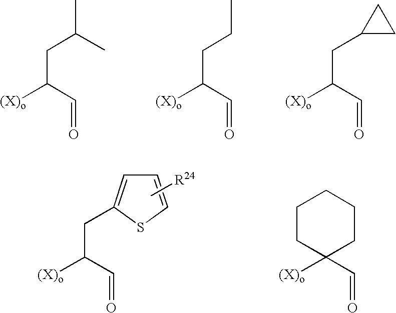
wherein R²⁴ is chosen from hydrogen, methyl, methoxy, ethyl, isopropyl, F, Cl and wherein any of the alkyl groups may be substituted with one or more F or Cl.

In order to maximise the inhibition of cathepsin K, the compound of formula (I) may comprise R¹¹ groups which are simple branched alkyl groups such as isobutyl or n-propyl or halogen substituted variants thereof such as 3,3,3-trifluoro-2-trifluoromethylpropyl.

In compounds of general formula (I) that are inhibitors of cathepsin K, it is preferred that the group U comprises an optionally substituted 5- or 6-membered saturated or unsaturated heterocycle or Ar group or an optionally substituted saturated or unsaturated 8 to 10-membered heterocycle or Ar group. Examples of such preferred U rings include, but are not limited to the following: 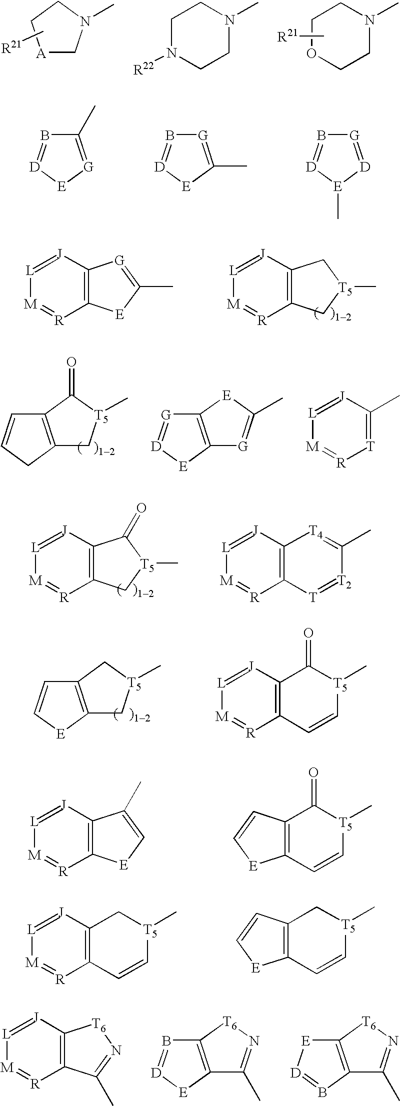
wherein R²¹, R²², A, B, D, E, G, J, L, M, R, T, T₂, T₄, T₅ and T₆ are as defined previously.

Stronger inhibition of cathepsin K can be achieved in compounds where the U groups comprise a bulky alkyl or aryl group at the para position of an aryl; a meta or para 5,6-biaryl Ar—Ar, where Ar is as previously defined; a 6,6 or 6,5 or 5,5-fused aromatic ring, where Ar is as previously defined, or a 4-substituted piperazine. Examples of more preferred U groups include but are not limited to: 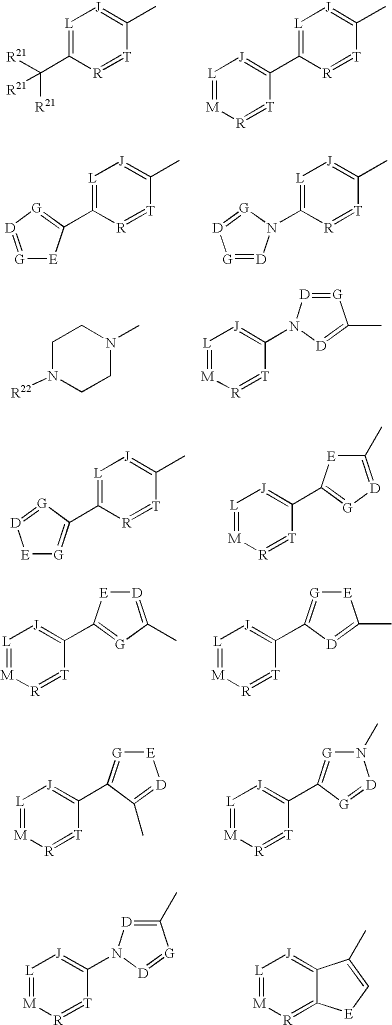 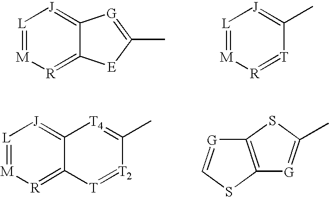
wherein R²¹, R²², D, E, G, J, L, M, R, T, T₂ and T₄ are as defined previously.

In compounds that are inhibitors of cathepsin K, even more preferred U groups, comprise a 6-membered aromatic ring Ar containing a bulky alkyl or aryl group at the para position; a meta or para-biaryl Ar—Ar, where Ar is as previously defined; a 6,6 or 6,5 or 5,5-fused aromatic ring, where Ar is as previously defined; or a 4-substituted piperazine where R²⁵ is chosen from hydrogen, C₁₋₂-alkyl or Ar—C₀₋₂-alkyl. Examples of even more preferred U groups include but are not limited to: 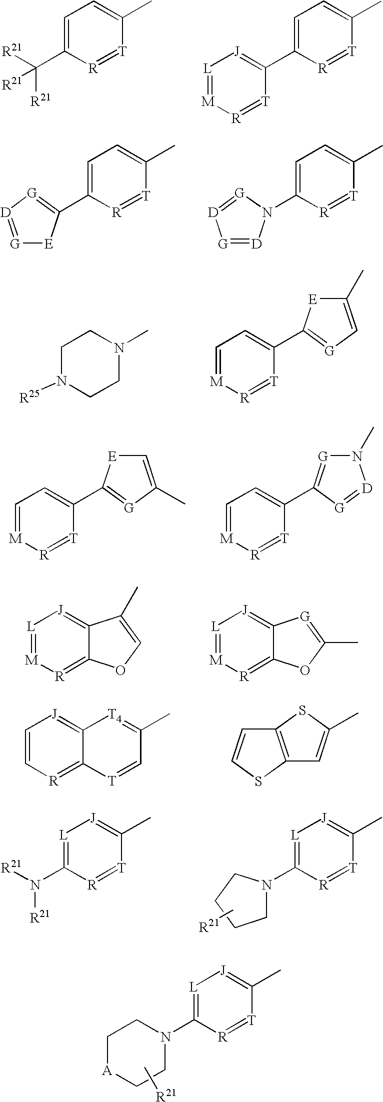
wherein R²¹, R²⁵, D, E, G, J, L, M, R, T and T₄ are as defined previously.

In order to maximise inhibition of cathepsin K, compounds of general formula (I) may be selected to have U groups chosen from the following: 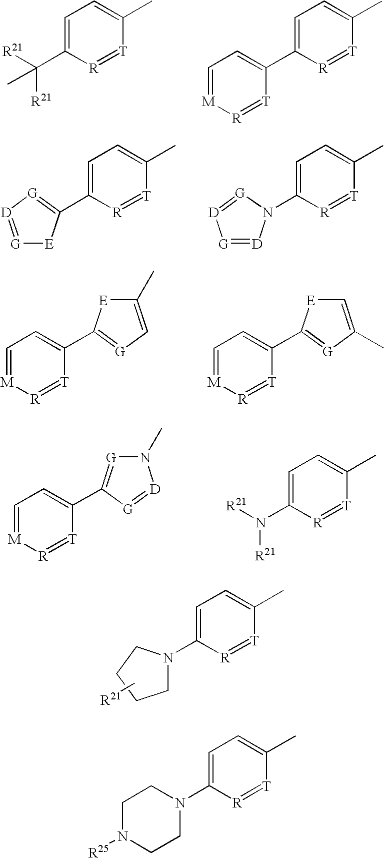
wherein R²¹, R²⁵, D, E, G, M, R and T are as defined previously.

In order to achieve the greatest inhibitory effect against cathepsin S, it is preferred that the Y substituent is chosen from the following; 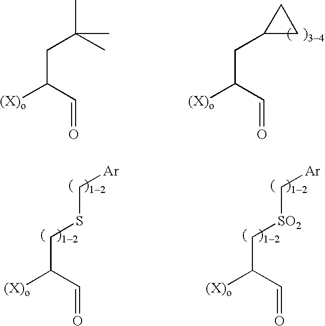
wherein (X)_{o′} and Ar are as previously defined.

In order to achieve the greatest inhibitory effect against cathepsin S, it is preferred that the group U comprises an optionally substituted 5-membered unsaturated heterocycle or a 6,5- 5,5- or 5,6-fused aromatic ring, where Ar is as previously defined or a morpholine. Examples of such preferred U rings include, but are not limited to the following: 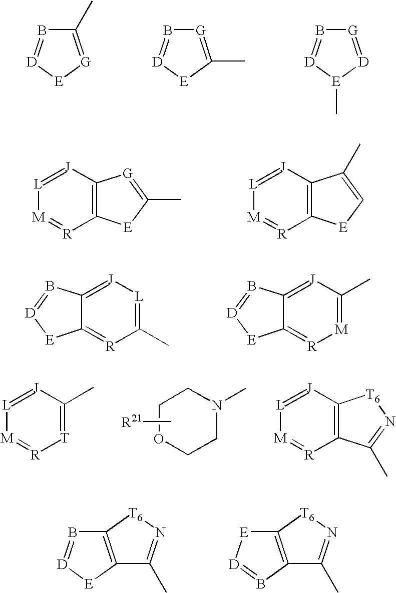
wherein R²¹, B, D, E, G, J, L, M, R and T₆ are as defined previously.

In order to achieve the greatest inhibitory effect against cathepsin S whilst retaining selectivity against other CA C1 cysteinyl proteases, it is more preferred that the group U comprises an optionally substituted 5-membered unsaturated heterocycle or a 6,5- or 5,5-fused aromatic ring, where Ar is as previously defined. Examples of more preferred U rings include, but are not limited to the following: 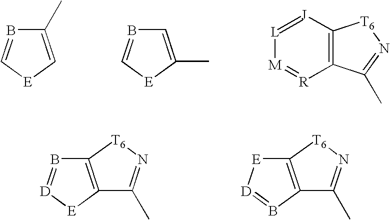
wherein B, D, E, J, L, M, R and T₆ are as defined previously.

In order to achieve the greatest inhibitory effect against cathepsin L, it is preferred that the Y substituent is chosen as an aromatic group as follows; 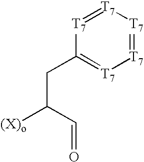
wherein T₇ is chosen from CH, N or CR²¹ where R²¹ is as defined previously.

In particular, for cathepsin L inhibition it is more preferred that within the T₇ substituent that the R²¹ substituent is chosen from single and multiple ring substitution combinations of Me, F, Cl, OH and OMe.

In order to achieve the greatest inhibitory effect against cathepsin L, it is preferred that the group U comprises an optionally substituted 5-membered unsaturated heterocycle or a 6,6- or 6,5- or 5,6-fused aromatic ring, where Ar is as previously defined or a meta-substituted Ar. Examples of such preferred U rings include, but are not limited to the following: 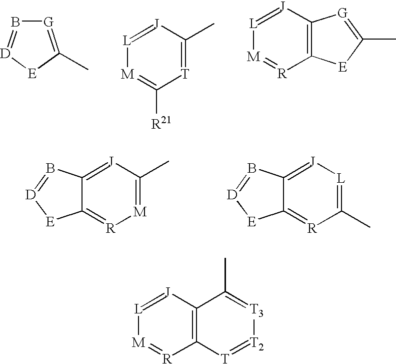
wherein R²¹, B, D, E, G. J, L, M, R, T, T₂ and T₃ are as defined previously.

In order to achieve the greatest inhibitory effect against cathepsin L whilst retaining selectivity against other CA C1 cysteinyl proteases, it is more preferred that the group U comprises a substituted 5-membered unsaturated heterocycle or a 6,6-fused aromatic ring, where Ar is as previously defined or a meta-substituted Ar. Examples of such preferred U rings include, but are not limited to the following: 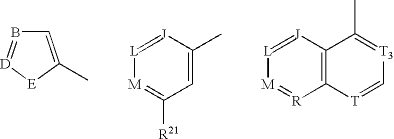
wherein E is chosen from oxygen or N-ethyl, D is chosen from nitrogen or CCH₃, B is chosen from nitrogen or CCH₃, R²¹ is chosen from halogen, OMe, CF₃, OCF₃, CH₂NH₂ and J, L, M, R, T and T₃ are as previously defined.

The inventors have observed that for the cruzipains and leismania mexicana CPB protease, that the U and Y substituent preferences are composed of a mixture of those described earlier for cathepsin K and cathepsin L. In essence, many of the preferred cathepsin K and cathepsin L inhibitors also show potency against the cruzipains and leismania mexicana CPB protease as highlighted in the EXAMPLES section. Such promiscurity can be used to provide potent and selective inhibitors of the cruzipains and leismania meicana CPB protease by combining a preferred U substituent as described for cathepsin K with a preferred Y substituent as described for cathepsin L or by combining a preferred U substituent as described for cathepsin L with a preferred Y substituent as described for cathepsin K Such preferred combinations provide potent inhibitors of the cruzipains and leismania mexicana CPB protease with selectivity against either or both cathepsin K and cathepsin L.

Particular compounds of the invention are selected from the compounds formed by joining one of the ‘U—(V)ₘ’ fragments herein defined as the ‘Capping group (Cg1 to Cg103)’ of general formula (I) shown in Table 1, with one of the ‘(W)ₙ—(X)ₒ—Y’ fragments herein defined as the ‘P2 pocket group (Pg1 to Pg39)’ of general formula (I) shown in Table 2, with a 5,5-bicyclic scaffold containing one of the R¹ fragments herein defined as the ‘Prime-side binding group (Ps1 to Ps243)’ of general formula (I) shown in Table 3.

TABLE 1
‘Capping group Cg’ Fragments

| | U—(V)ₘ |
|---|---|
| Cg1 | 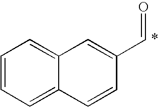 |
| Cg2 | 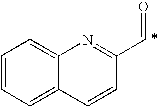 |
| Cg3 | 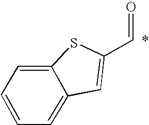 |
| Cg4 | 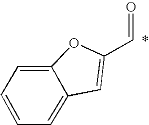 |
| Cg5 | 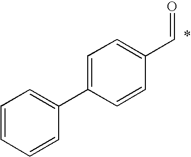 |
| Cg6 | 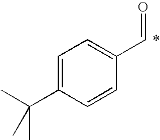 |
| Cg7 | 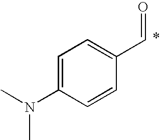 |
| Cg8 | 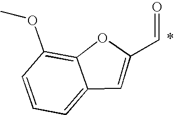 |
| Cg9 | 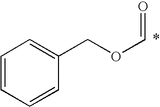 |
| Cg10 | 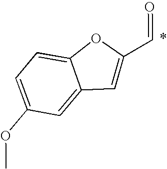 |
| Cg11 | 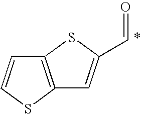 |
| Cg12 | 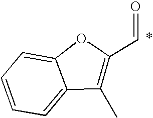 |
| Cg13 | 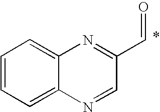 |
| Cg14 | 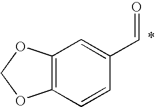 |
| Cg15 | 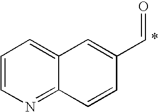 |
| Cg16 | 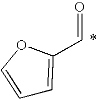 |
| Cg17 | 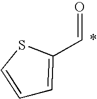 |
| Cg18 | 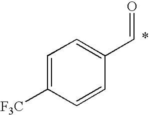 |
| Cg19 | 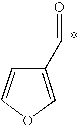 |
| Cg20 | 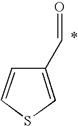 |
| Cg21 | 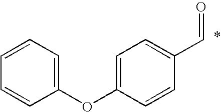 |
| Cg22 | 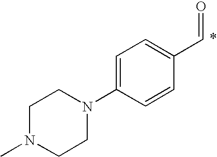 |
| Cg23 | 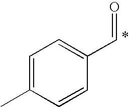 |
| Cg24 | 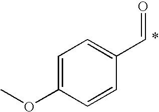 |
| Cg25 | 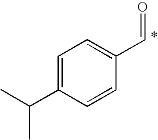 |
| Cg26 | 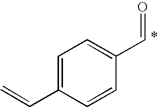 |
| Cg27 | 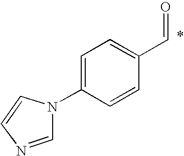 |
| Cg28 | 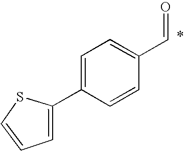 |
| Cg29 | 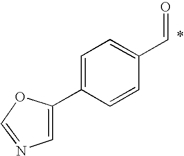 |
| Cg30 | 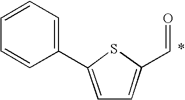 |
| Cg31 | 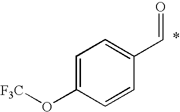 |
| Cg32 | 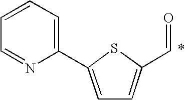 |
| Cg33 | 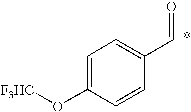 |
| Cg34 | 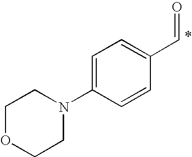 |
| Cg35 | 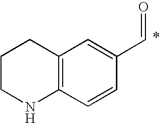 |
| Cg36 | 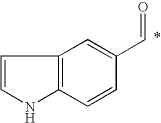 |
| Cg37 | 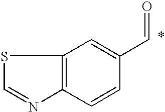 |
| Cg38 | 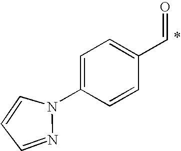 |
| Cg39 | 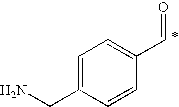 |
| Cg40 | 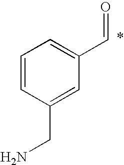 |
| Cg41 | 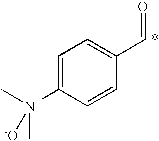 |
| Cg42 | 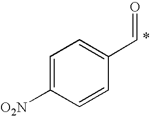 |
| Cg43 | 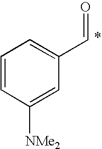 |
| Cg44 | 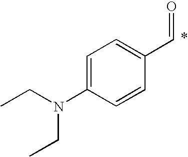 |
| Cg45 | 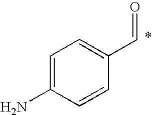 |
| Cg46 | 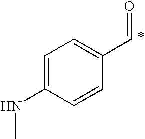 |
| Cg47 | 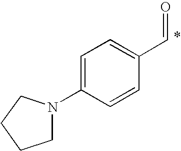 |
| Cg48 | 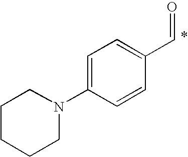 |
| Cg49 | 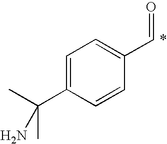 |
| Cg50 | 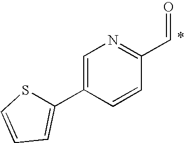 |
| Cg51 | 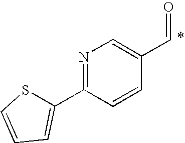 |
| Cg52 | 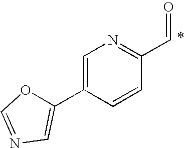 |
| Cg53 | 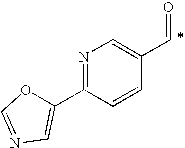 |
| Cg54 | 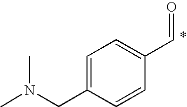 |
| Cg55 | 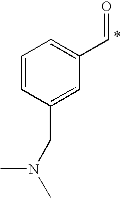 |
| Cg56 | 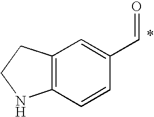 |
| Cg57 | 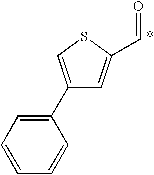 |
| Cg58 | 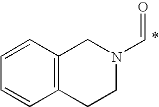 |
| Cg59 | 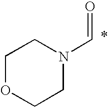 |
| Cg60 | 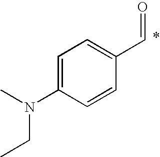 |
| Cg61 | 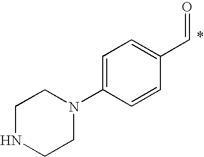 |
| Cg62 | 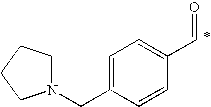 |
| Cg63 | 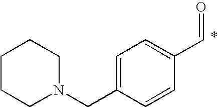 |
| Cg64 | 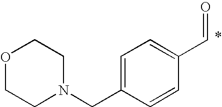 |
| Cg65 | 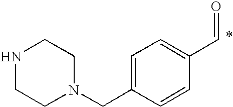 |
| Cg66 | 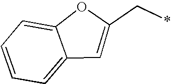 |
| Cg67 | 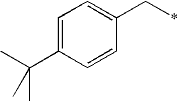 |
| Cg68 | 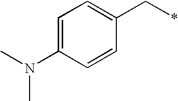 |
| Cg69 | 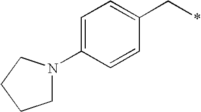 |
| Cg70 | 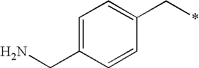 |
| Cg71 | 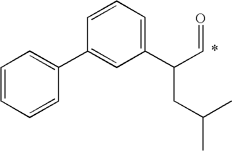 |
| Cg72 | 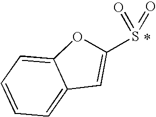 |
| Cg73 | 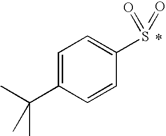 |
| Cg74 | 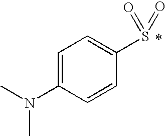 |
| Cg75 | 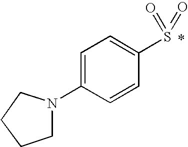 |
| Cg76 | 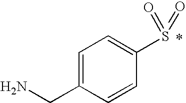 |
| Cg77 | 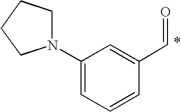 |
| Cg78 | 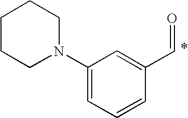 |
| Cg79 | 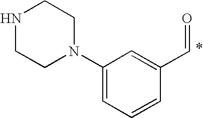 |
| Cg80 | 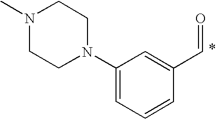 |
| Cg81 | 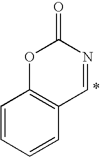 |
| Cg82 | 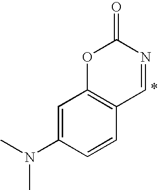 |
| Cg83 | 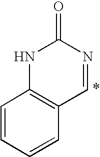 |
| Cg84 | 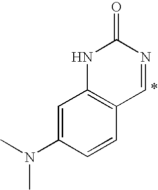 |
| Cg85 | 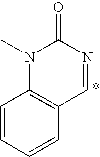 |
| Cg86 | 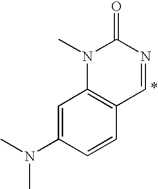 |
| Cg87 | 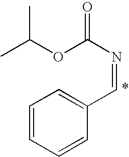 |
| Cg88 | 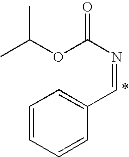 |
| Cg89 | 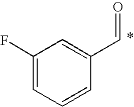 |
| Cg90 | 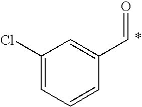 |
| Cg91 | 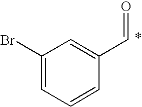 |
| Cg92 | 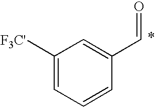 |
| Cg93 | 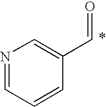 |
| Cg94 | 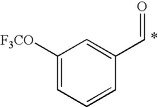 |
| Cg95 | 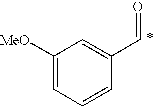 |
| Cg96 | 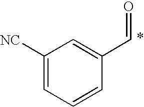 |
| Cg97 | 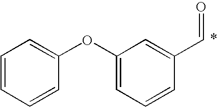 |
| Cg98 | 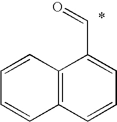 |
| Cg99 | 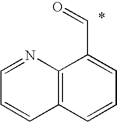 |
| Cg100 | 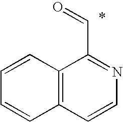 |
| Cg101 | 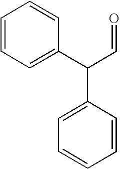 |
| Cg102 | 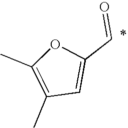 |
| Cg103 | 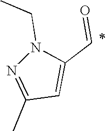 |

*signifies the point of attachment of ’Cg groups’ to ‘Pg groups’.

TABLE 2
‘P2 pocket group Pg’ Fragments

| | (W)ₙ—(X)ₒ—Y |
|---|---|
| Pg1 | 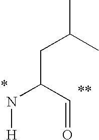 |
| Pg2 | 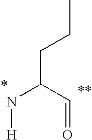 |
| Pg3 | 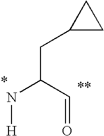 |
| Pg4 | 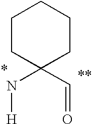 |
| Pg5 | 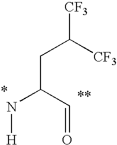 |
| Pg6 | 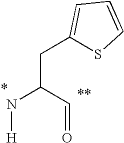 |
| Pg7 | 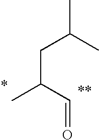 |
| Pg8 | 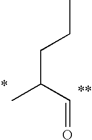 |
| Pg9 | 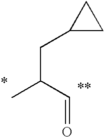 |
| Pg10 | 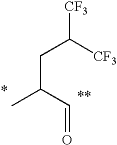 |
| Pg11 | 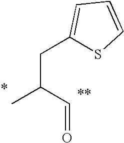 |
| Pg12 | 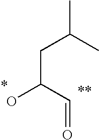 |
| Pg13 | 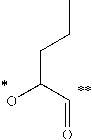 |
| Pg14 | 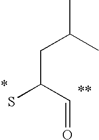 |
| Pg15 | 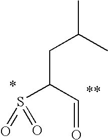 |
| Pg16 | 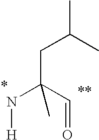 |
| Pg17 | 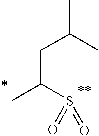 |
| Pg18 | 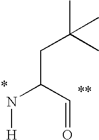 |
| Pg19 | 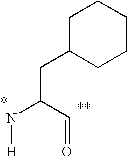 |
| Pg20 | 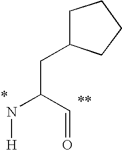 |
| Pg21 | 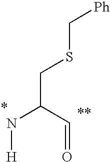 |
| Pg22 | 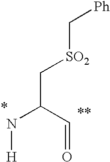 |
| Pg23 | 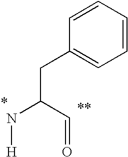 |
| Pg24 | 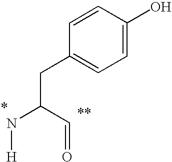 |
| Pg25 | 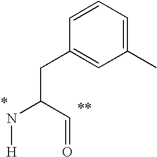 |
| Pg26 | 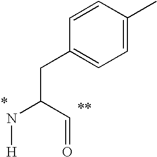 |
| Pg27 | 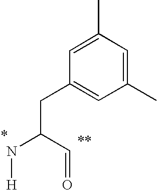 |
| Pg28 | 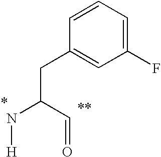 |
| Pg29 | 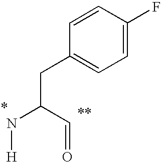 |
| Pg30 | 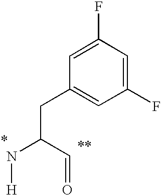 |
| Pg31 | 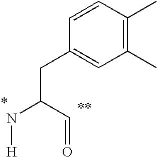 |
| Pg32 | 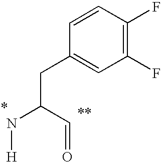 |
| Pg33 | 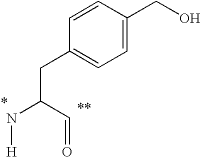 |
| Pg34 | 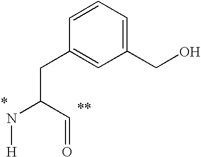 |
| Pg35 | 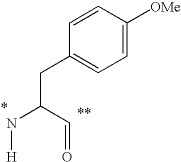 |
| Pg36 | 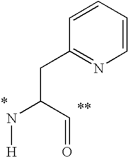 |
| Pg37 | 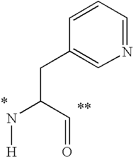 |
| Pg38 | 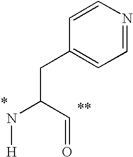 |
| Pg39 | 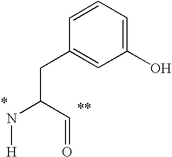 |

**signifies the point of attachment of ‘Pg groups’ to 5,5-bicyclic scaffold.

TABLE 3
‘Prime-side binding group Ps’ Fragments

| | R₁ |
|---|---|
| Ps1 | 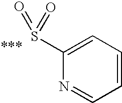 |
| Ps2 | 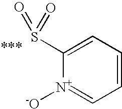 |
| Ps3 | 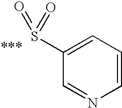 |
| Ps4 | 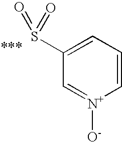 |
| Ps5 | 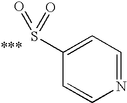 |
| Ps6 | 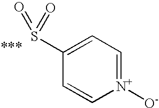 |
| Ps7 | 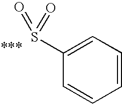 |
| Ps8 | 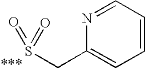 |
| Ps9 | 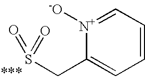 |
| Ps10 | 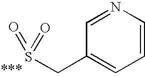 |
| Ps11 | 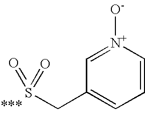 |
| Ps12 | 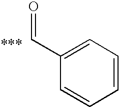 |
| Ps13 | 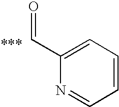 |
| Ps14 | 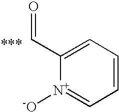 |
| Ps15 | 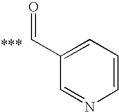 |
| Ps16 | 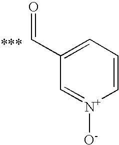 |
| Ps17 | 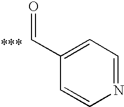 |
| Ps18 | 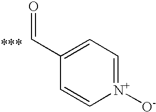 |
| Ps19 | 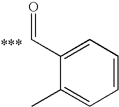 |
| Ps20 | 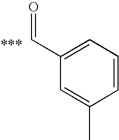 |
| Ps21 | 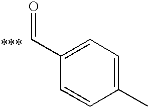 |
| Ps22 | 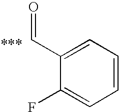 |
| Ps23 | 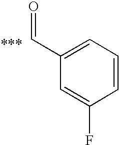 |
| Ps24 | 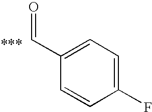 |
| Ps25 | 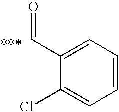 |
| Ps26 | 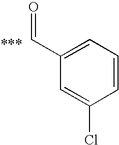 |
| Ps27 | 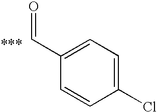 |
| Ps28 | 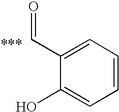 |
| Ps29 | 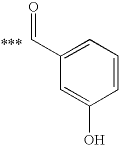 |
| Ps30 | 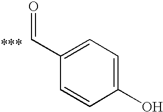 |
| Ps31 | 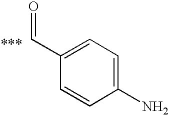 |
| Ps32 | 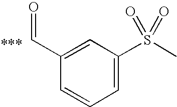 |
| Ps33 | 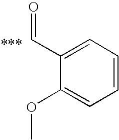 |
| Ps34 | 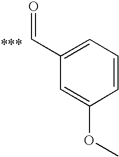 |
| Ps35 | 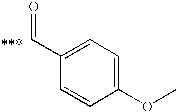 |
| Ps36 | 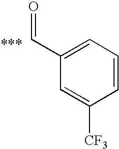 |
| Ps37 | 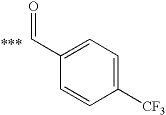 |
| Ps38 | 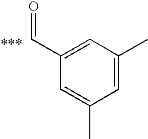 |
| Ps39 | 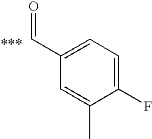 |
| Ps40 | 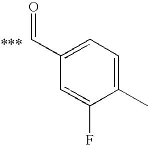 |
| Ps41 | 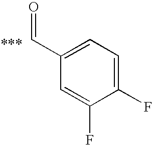 |
| Ps42 | 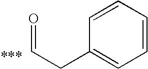 |
| Ps43 | 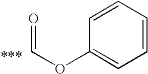 |
| Ps44 | 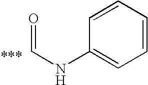 |
| Ps45 | 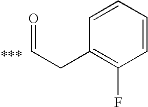 |
| Ps46 | 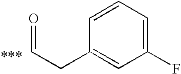 |
| Ps47 | 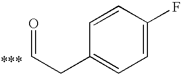 |
| Ps48 | 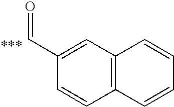 |
| Ps49 | 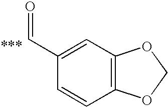 |
| Ps50 | 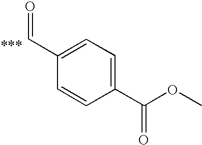 |
| Ps51 | 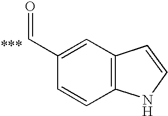 |
| Ps52 | 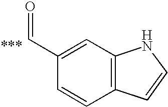 |
| Ps53 | 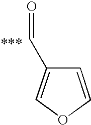 |
| Ps54 | 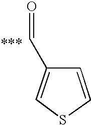 |
| Ps55 | 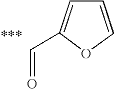 |
| Ps56 | 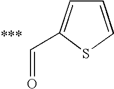 |
| Ps57 | 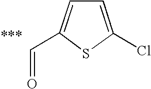 |
| Ps58 | 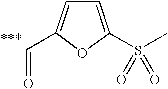 |
| Ps59 | 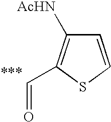 |
| Ps60 | 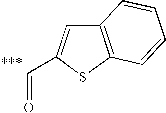 |
| Ps61 | 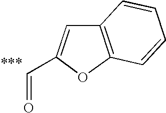 |
| Ps62 | 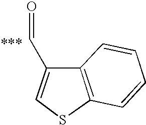 |
| Ps63 | 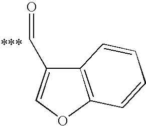 |
| Ps64 | 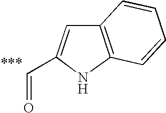 |
| Ps65 | 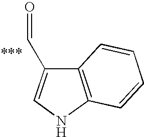 |
| Ps66 | 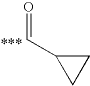 |
| Ps67 | 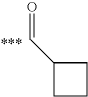 |
| Ps68 | 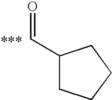 |
| Ps69 | 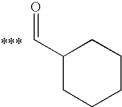 |
| Ps70 | 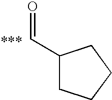 |
| Ps71 | 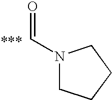 |
| Ps72 | 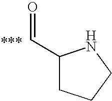 |
| Ps73 | 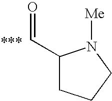 |
| Ps74 | 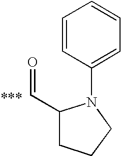 |
| Ps75 | 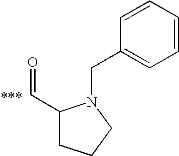 |
| Ps76 | 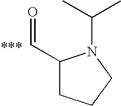 |
| Ps77 | 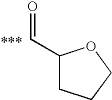 |
| Ps78 | 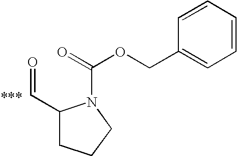 |
| Ps79 | 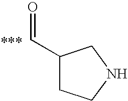 |
| Ps80 | 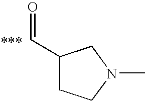 |
| Ps81 | 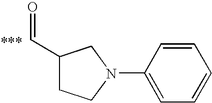 |
| Ps82 | 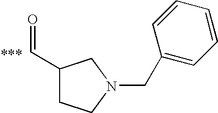 |
| Ps83 | 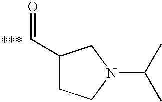 |
| Ps84 | 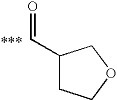 |
| Ps85 | 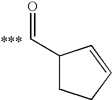 |
| Ps86 | 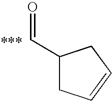 |
| Ps87 | 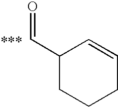 |
| Ps88 | 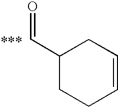 |
| Ps89 | 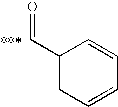 |
| Ps90 | 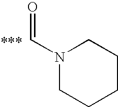 |
| Ps91 | 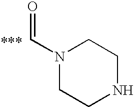 |
| Ps92 | 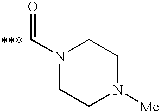 |
| Ps93 | 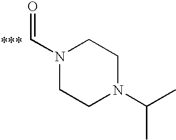 |
| Ps94 | 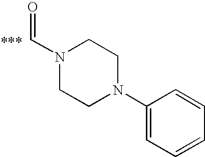 |
| Ps95 | 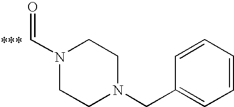 |
| Ps96 | 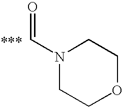 |
| Ps97 | 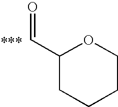 |
| Ps98 | 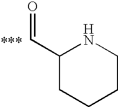 |
| Ps99 | 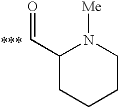 |
| Ps100 | 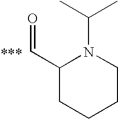 |
| Ps101 | 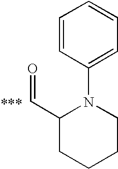 |
| Ps102 | 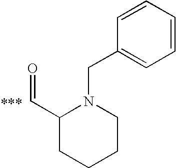 |
| Ps103 | 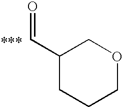 |
| Ps104 | 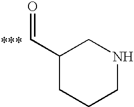 |
| Ps105 | 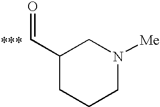 |
| Ps106 | 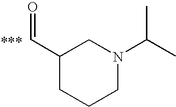 |
| Ps107 | 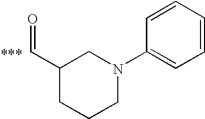 |
| Ps108 | 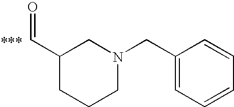 |
| Ps109 | 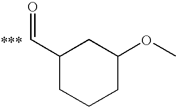 |
| Ps110 | 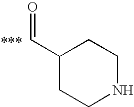 |
| Ps111 | 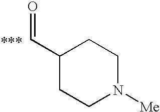 |
| Ps112 | 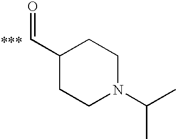 |
| Ps113 | 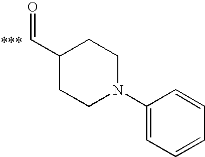 |
| Ps114 | 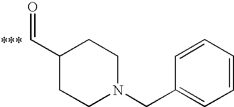 |
| Ps115 | 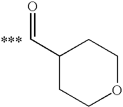 |
| Ps116 | 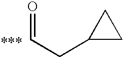 |
| Ps117 | 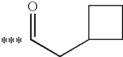 |
| Ps118 | 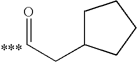 |
| Ps119 | 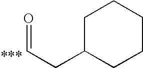 |
| Ps120 | 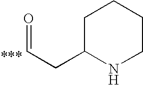 |
| Ps121 | 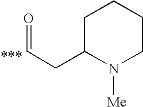 |
| Ps122 | 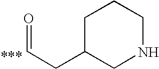 |
| Ps123 | 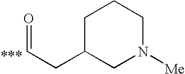 |
| Ps124 | 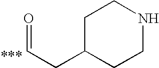 |
| Ps125 | 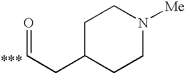 |
| Ps126 | 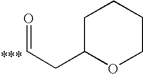 |
| Ps127 | 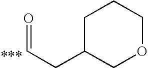 |
| Ps128 | 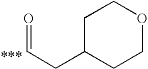 |
| Ps129 | 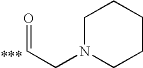 |
| Ps130 | 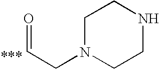 |
| Ps131 | 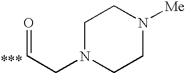 |
| Ps132 | 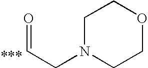 |
| Ps133 | 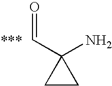 |
| Ps134 | 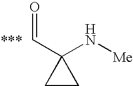 |
| Ps135 | 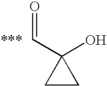 |
| Ps136 | 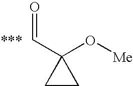 |
| Ps137 | 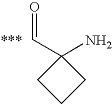 |
| Ps138 | 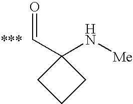 |
| Ps139 | 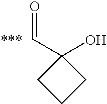 |
| Ps140 | 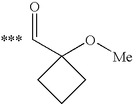 |
| Ps141 | 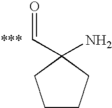 |
| Ps142 | 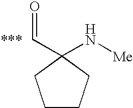 |
| Ps143 | 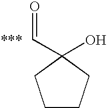 |
| Ps144 | 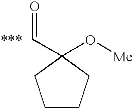 |
| Ps145 | 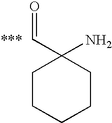 |
| Pd146 | 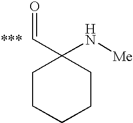 |
| Ps147 | 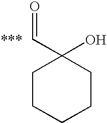 |
| Ps148 | 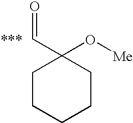 |
| Ps149 | 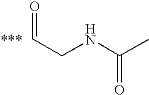 |
| Ps150 | 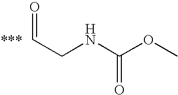 |
| Ps151 | 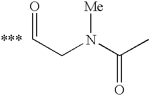 |
| Ps152 | 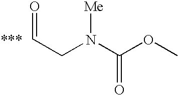 |
| Ps153 | 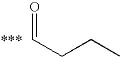 |
| Ps154 | 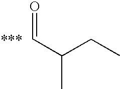 |
| Ps155 | 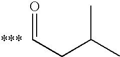 |
| Ps156 | 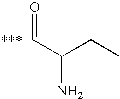 |
| Ps157 | 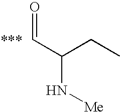 |
| Ps158 | 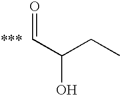 |
| Ps159 | 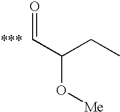 |
| Ps160 | 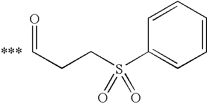 |
| Ps161 | 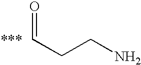 |
| Ps162 | 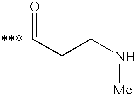 |
| Ps163 | 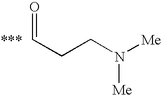 |
| Ps164 | 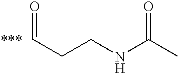 |
| Ps165 | 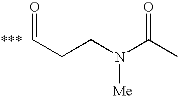 |
| Ps166 | 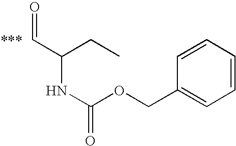 |
| Ps167 | 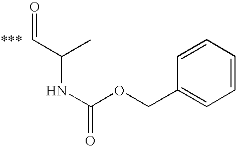 |
| Ps168 | 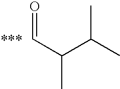 |
| Ps169 | 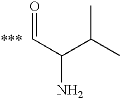 |
| Ps170 | 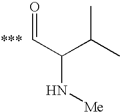 |
| Ps171 | 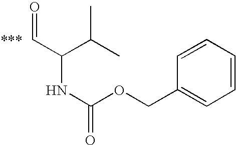 |
| Ps172 | 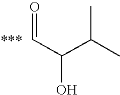 |
| Ps173 | 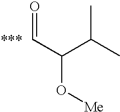 |
| Ps174 | 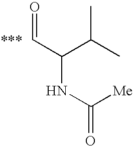 |
| Ps175 | 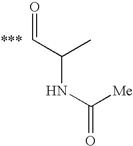 |
| Ps176 | 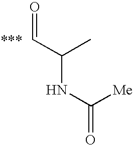 |
| Ps177 | 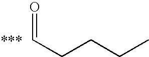 |
| Ps178 | 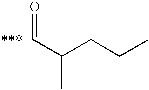 |
| Ps179 | 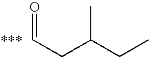 |
| Ps180 | 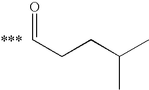 |
| Ps181 | 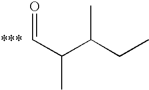 |
| Ps182 | 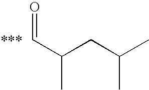 |
| Ps183 | 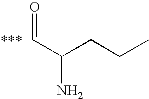 |
| Ps184 | 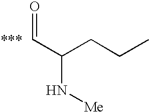 |
| Ps185 | 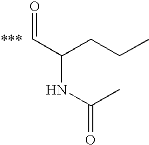 |
| Ps186 | 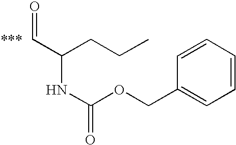 |
| Ps187 | 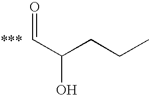 |
| Ps188 | 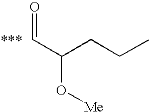 |
| Ps189 | 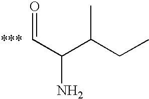 |
| Ps190 | 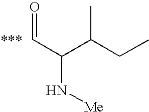 |
| Ps191 | 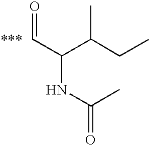 |
| Ps192 | 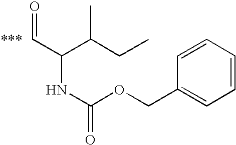 |
| Ps193 | 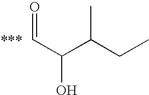 |
| Ps194 | 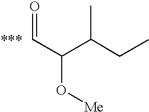 |
| Ps195 | 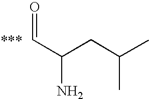 |
| Ps196 | 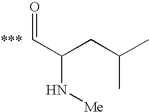 |
| Ps197 | 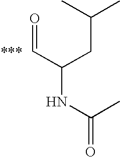 |
| Ps198 | 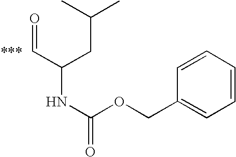 |
| Ps199 | 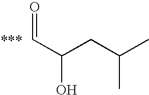 |
| Ps200 | 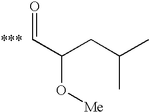 |
| Ps201 | 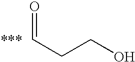 |
| Ps202 | 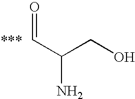 |
| Ps203 | 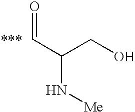 |
| Ps204 | 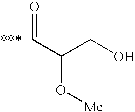 |
| Ps205 | 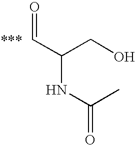 |
| Ps206 | 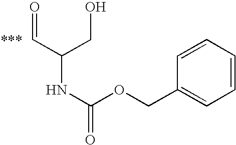 |
| Ps207 | 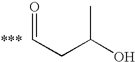 |
| Ps208 | 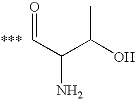 |
| Ps209 | 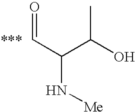 |
| Ps210 | 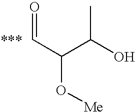 |
| Ps211 | 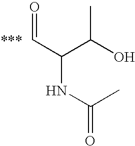 |
| Ps212 | 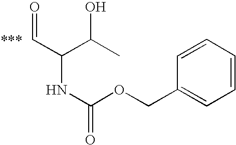 |
| Ps213 | 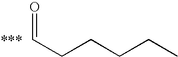 |
| Ps214 | 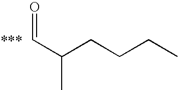 |
| Ps215 | 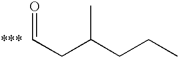 |
| Ps216 | 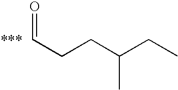 |
| Ps217 | 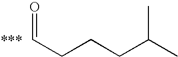 |
| Ps218 | 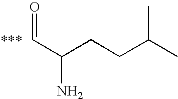 |
| Ps219 | 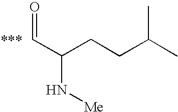 |
| Ps220 | 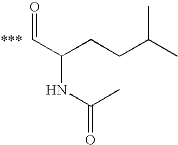 |
| Ps221 | 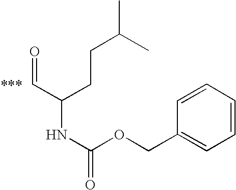 |
| Ps222 | 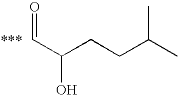 |
| Ps223 | 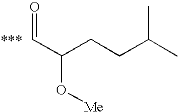 |
| Ps224 | 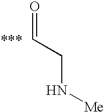 |
| Ps225 | 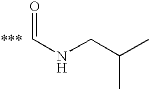 |
| Ps226 | 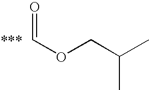 |
| Ps227 | 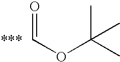 |
| Ps228 | 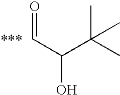 |
| Ps229 | 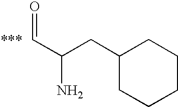 |
| Ps230 | 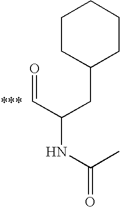 |
| Ps231 | 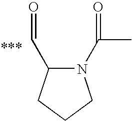 |
| Ps232 | 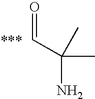 |
| Ps233 | 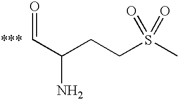 |
| Ps234 | 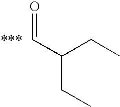 |
| Ps235 | 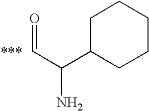 |
| Ps236 | 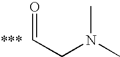 |
| Ps237 | 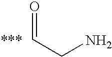 |
| Ps238 | 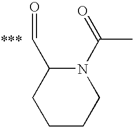 |
| Ps239 | 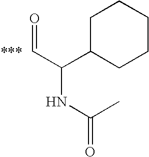 |
| Ps240 | 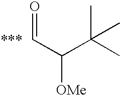 |
| Ps241 | 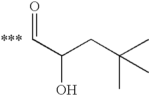 |
| Ps242 | 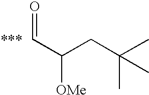 |
| Ps243 | 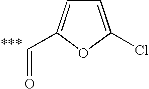 |

***signifies the point of attachment of ‘Ps groups’ to 5,5-bicyclic scaffold.

Particularly preferred compounds of the invention are inhibitors of cathepsin K and include but are not limited to the compounds formed by the following Cg-Pg-Ps combinations;

| | | | |
|---|---|---|---|
| Cg5-Pg1-Ps1 | Cg5-Pg1-Ps2 | Cg5-Pg1-Ps3 | Cg5-Pg1-Ps4 |
| Cg5-Pg1-Ps5 | Cg5-Pg1-Ps6 | Cg5-Pg1-Ps7 | Cg5-Pg1-Ps8 |
| Cg5-Pg1-Ps9 | Cg5-Pg1-Ps10 | Cg5-Pg1-Ps11 | Cg5-Pg1-Ps12 |
| Cg5-Pg1-Ps13 | Cg5-Pg1-Ps14 | Cg5-Pg1-Ps15 | Cg5-Pg1-Ps16 |
| Cg5-Pg1-Ps17 | Cg5-Pg1-Ps18 | Cg5-Pg1-Ps19 | Cg5-Pg1-Ps20 |
| Cg5-Pg1-Ps21 | Cg5-Pg1-Ps22 | Cg5-Pg1-Ps23 | Cg5-Pg1-Ps24 |
| Cg5-Pg1-Ps25 | Cg5-Pg1-Ps26 | Cg5-Pg1-Ps27 | Cg5-Pg1-Ps28 |
| Cg5-Pg1-Ps29 | Cg5-Pg1-Ps30 | Cg5-Pg1-Ps31 | Cg5-Pg1-Ps32 |
| Cg5-Pg1-Ps33 | Cg5-Pg1-Ps34 | Cg5-Pg1-Ps35 | Cg5-Pg1-Ps36 |
| Cg5-Pg1-Ps37 | Cg5-Pg1-Ps38 | Cg5-Pg1-Ps39 | Cg5-Pg1-Ps40 |
| Cg5-Pg1-Ps41 | Cg5-Pg1-Ps42 | Cg5-Pg1-Ps43 | Cg5-Pg1-Ps44 |
| Cg5-Pg1-Ps45 | Cg5-Pg1-Ps46 | Cg5-Pg1-Ps47 | Cg5-Pg1-Ps48 |
| Cg5-Pg1-Ps49 | Cg5-Pg1-Ps50 | Cg5-Pg1-Ps51 | Cg5-Pg1-Ps52 |
| Cg5-Pg1-Ps53 | Cg5-Pg1-Ps54 | Cg5-Pg1-Ps55 | Cg5-Pg1-Ps56 |
| Cg5-Pg1-Ps57 | Cg5-Pg1-Ps58 | Cg5-Pg1-Ps59 | Cg5-Pg1-Ps60 |
| Cg5-Pg1-Ps61 | Cg5-Pg1-Ps62 | Cg5-Pg1-Ps63 | Cg5-Pg1-Ps64 |
| Cg5-Pg1-Ps65 | Cg5-Pg1-Ps66 | Cg5-Pg1-Ps67 | Cg5-Pg1-Ps68 |
| Cg5-Pg1-Ps69 | Cg5-Pg1-Ps70 | Cg5-Pg1-Ps71 | Cg5-Pg1-Ps72 |
| Cg5-Pg1-Ps73 | Cg5-Pg1-Ps74 | Cg5-Pg1-Ps75 | Cg5-Pg1-Ps76 |
| Cg5-Pg1-Ps77 | Cg5-Pg1-Ps78 | Cg5-Pg1-Ps79 | Cg5-Pg1-Ps80 |
| Cg5-Pg1-Ps81 | Cg5-Pg1-Ps82 | Cg5-Pg1-Ps83 | Cg5-Pg1-Ps84 |
| Cg5-Pg1-Ps85 | Cg5-Pg1-Ps86 | Cg5-Pg1-Ps87 | Cg5-Pg1-Ps88 |
| Cg5-Pg1-Ps89 | Cg5-Pg1-Ps90 | Cg5-Pg1-Ps91 | Cg5-Pg1-Ps92 |
| Cg5-Pg1-Ps93 | Cg5-Pg1-Ps94 | Cg5-Pg1-Ps95 | Cg5-Pg1-Ps96 |
| Cg5-Pg1-Ps97 | Cg5-Pg1-Ps98 | Cg5-Pg1-Ps99 | Cg5-Pg1-Ps100 |
| Cg5-Pg1-Ps101 | Cg5-Pg1-Ps102 | Cg5-Pg1-Ps103 | Cg5-Pg1-Ps104 |
| Cg5-Pg1-Ps105 | Cg5-Pg1-Ps106 | Cg5-Pg1-Ps107 | Cg5-Pg1-Ps108 |
| Cg5-Pg1-Ps109 | Cg5-Pg1-Ps110 | Cg5-Pg1-Ps111 | Cg5-Pg1-Ps112 |
| Cg5-Pg1-Ps113 | Cg5-Pg1-Ps114 | Cg5-Pg1-Ps115 | Cg5-Pg1-Ps116 |
| Cg5-Pg1-Ps117 | Cg5-Pg1-Ps118 | Cg5-Pg1-Ps119 | Cg5-Pg1-Ps120 |
| Cg5-Pg1-Ps121 | Cg5-Pg1-Ps122 | Cg5-Pg1-Ps123 | Cg5-Pg1-Ps124 |
| Cg5-Pg1-Ps125 | Cg5-Pg1-Ps126 | Cg5-Pg1-Ps127 | Cg5-Pg1-Ps128 |
| Cg5-Pg1-Ps129 | Cg5-Pg1-Ps130 | Cg5-Pg1-Ps131 | Cg5-Pg1-Ps132 |
| Cg5-Pg1-Ps133 | Cg5-Pg1-Ps134 | Cg5-Pg1-Ps135 | Cg5-Pg1-Ps136 |
| Cg5-Pg1-Ps137 | Cg5-Pg1-Ps138 | Cg5-Pg1-Ps139 | Cg5-Pg1-Ps140 |
| Cg5-Pg1-Ps141 | Cg5-Pg1-Ps142 | Cg5-Pg1-Ps143 | Cg5-Pg1-Ps144 |
| Cg5-Pg1-Ps145 | Cg5-Pg1-Ps146 | Cg5-Pg1-Ps147 | Cg5-Pg1-Ps148 |
| Cg5-Pg1-Ps149 | Cg5-Pg1-Ps150 | Cg5-Pg1-Ps151 | Cg5-Pg1-Ps152 |
| Cg5-Pg1-Ps153 | Cg5-Pg1-Ps154 | Cg5-Pg1-Ps155 | Cg5-Pg1-Ps156 |
| Cg5-Pg1-Ps157 | Cg5-Pg1-Ps158 | Cg5-Pg1-Ps159 | Cg5-Pg1-Ps160 |
| Cg5-Pg1-Ps161 | Cg5-Pg1-Ps162 | Cg5-Pg1-Ps163 | Cg5-Pg1-Ps164 |
| Cg5-Pg1-Ps165 | Cg5-Pg1-Ps166 | Cg5-Pg1-Ps167 | Cg5-Pg1-Ps168 |
| Cg5-Pg1-Ps169 | Cg5-Pg1-Ps170 | Cg5-Pg1-Ps171 | Cg5-Pg1-Ps172 |
| Cg5-Pg1-Ps173 | Cg5-Pg1-Ps174 | Cg5-Pg1-Ps175 | Cg5-Pg1-Ps176 |
| Cg5-Pg1-Ps177 | Cg5-Pg1-Ps178 | Cg5-Pg1-Ps179 | Cg5-Pg1-Ps180 |
| Cg5-Pg1-Ps181 | Cg5-Pg1-Ps182 | Cg5-Pg1-Ps183 | Cg5-Pg1-Ps184 |
| Cg5-Pg1-Ps185 | Cg5-Pg1-Ps186 | Cg5-Pg1-Ps187 | Cg5-Pg1-Ps188 |
| Cg5-Pg1-Ps189 | Cg5-Pg1-Ps190 | Cg5-Pg1-Ps191 | Cg5-Pg1-Ps192 |
| Cg5-Pg1-Ps193 | Cg5-Pg1-Ps194 | Cg5-Pg1-Ps195 | Cg5-Pg1-Ps196 |
| Cg5-Pg1-Ps197 | Cg5-Pg1-Ps198 | Cg5-Pg1-Ps199 | Cg5-Pg1-Ps200 |
| Cg5-Pg1-Ps201 | Cg5-Pg1-Ps202 | Cg5-Pg1-Ps203 | Cg5-Pg1-Ps204 |
| Cg5-Pg1-Ps205 | Cg5-Pg1-Ps206 | Cg5-Pg1-Ps207 | Cg5-Pg1-Ps208 |
| Cg5-Pg1-Ps209 | Cg5-Pg1-Ps210 | Cg5-Pg1-Ps211 | Cg5-Pg1-Ps212 |
| Cg5-Pg1-Ps213 | Cg5-Pg1-Ps214 | Cg5-Pg1-Ps215 | Cg5-Pg1-Ps216 |
| Cg5-Pg1-Ps217 | Cg5-Pg1-Ps218 | Cg5-Pg1-Ps219 | Cg5-Pg1-Ps220 |
| Cg5-Pg1-Ps221 | Cg5-Pg1-Ps222 | Cg5-Pg1-Ps223 | Cg5-Pg1-Ps224 |
| Cg5-Pg1-Ps225 | Cg5-Pg1-Ps226 | Cg5-Pg1-Ps227 | Cg5-Pg1-Ps228 |
| Cg5-Pg1-Ps229 | Cg5-Pg1-Ps230 | Cg5-Pg1-Ps231 | Cg5-Pg1-Ps232 |
| Cg5-Pg1-Ps233 | Cg5-Pg1-Ps234 | Cg5-Pg1-Ps235 | Cg5-Pg1-Ps236 |
| Cg5-Pg1-Ps237 | Cg5-Pg1-Ps238 | Cg5-Pg1-Ps239 | Cg5-Pg1-Ps240 |
| Cg5-Pg1-Ps241 | Cg5-Pg1-Ps242 | Cg5-Pg1-Ps243 | |
| Cg6-Pg1-Ps1 | Cg6-Pg1-Ps2 | Cg6-Pg1-Ps3 | Cg6-Pg1-Ps4 |
| Cg6-Pg1-Ps5 | Cg6-Pg1-Ps6 | Cg6-Pg1-Ps7 | Cg6-Pg1-Ps8 |
| Cg6-Pg1-Ps9 | Cg6-Pg1-Ps10 | Cg6-Pg1-Ps11 | Cg6-Pg1-Ps12 |
| Cg6-Pg1-Ps13 | Cg6-Pg1-Ps14 | Cg6-Pg1-Ps15 | Cg6-Pg1-Ps16 |
| Cg6-Pg1-Ps17 | Cg6-Pg1-Ps18 | Cg6-Pg1-Ps19 | Cg6-Pg1-Ps20 |
| Cg6-Pg1-Ps21 | Cg6-Pg1-Ps22 | Cg6-Pg1-Ps23 | Cg6-Pg1-Ps24 |
| Cg6-Pg1-Ps25 | Cg6-Pg1-Ps26 | Cg6-Pg1-Ps27 | Cg6-Pg1-Ps28 |
| Cg6-Pg1-Ps29 | Cg6-Pg1-Ps30 | Cg6-Pg1-Ps31 | Cg6-Pg1-Ps32 |
| Cg6-Pg1-Ps33 | Cg6-Pg1-Ps34 | Cg6-Pg1-Ps35 | Cg6-Pg1-Ps36 |
| Cg6-Pg1-Ps37 | Cg6-Pg1-Ps38 | Cg6-Pg1-Ps39 | Cg6-Pg1-Ps40 |
| Cg6-Pg1-Ps41 | Cg6-Pg1-Ps42 | Cg6-Pg1-Ps43 | Cg6-Pg1-Ps44 |
| Cg6-Pg1-Ps45 | Cg6-Pg1-Ps46 | Cg6-Pg1-Ps47 | Cg6-Pg1-Ps48 |
| Cg6-Pg1-Ps49 | Cg6-Pg1-Ps50 | Cg6-Pg1-Ps51 | Cg6-Pg1-Ps52 |
| Cg6-Pg1-Ps53 | Cg6-Pg1-Ps54 | Cg6-Pg1-Ps55 | Cg6-Pg1-Ps56 |
| Cg6-Pg1-Ps57 | Cg6-Pg1-Ps58 | Cg6-Pg1-Ps59 | Cg6-Pg1-Ps60 |
| Cg6-Pg1-Ps61 | Cg6-Pg1-Ps62 | Cg6-Pg1-Ps63 | Cg6-Pg1-Ps64 |
| Cg6-Pg1-Ps65 | Cg6-Pg1-Ps66 | Cg6-Pg1-Ps67 | Cg6-Pg1-Ps68 |
| Cg6-Pg1-Ps69 | Cg6-Pg1-Ps70 | Cg6-Pg1-Ps71 | Cg6-Pg1-Ps72 |
| Cg6-Pg1-Ps73 | Cg6-Pg1-Ps74 | Cg6-Pg1-Ps75 | Cg6-Pg1-Ps76 |
| Cg6-Pg1-Ps77 | Cg6-Pg1-Ps78 | Cg6-Pg1-Ps79 | Cg6-Pg1-Ps80 |
| Cg6-Pg1-Ps81 | Cg6-Pg1-Ps82 | Cg6-Pg1-Ps83 | Cg6-Pg1-Ps84 |
| Cg6-Pg1-Ps85 | Cg6-Pg1-Ps86 | Cg6-Pg1-Ps87 | Cg6-Pg1-Ps88 |
| Cg6-Pg1-Ps89 | Cg6-Pg1-Ps90 | Cg6-Pg1-Ps91 | Cg6-Pg1-Ps92 |
| Cg6-Pg1-Ps93 | Cg6-Pg1-Ps94 | Cg6-Pg1-Ps95 | Cg6-Pg1-Ps96 |
| Cg6-Pg1-Ps97 | Cg6-Pg1-Ps98 | Cg6-Pg1-Ps99 | Cg6-Pg1-Ps100 |
| Cg6-Pg1-Ps101 | Cg6-Pg1-Ps102 | Cg6-Pg1-Ps103 | Cg6-Pg1-Ps104 |
| Cg6-Pg1-Ps105 | Cg6-Pg1-Ps106 | Cg6-Pg1-Ps107 | Cg6-Pg1-Ps108 |
| Cg6-Pg1-Ps109 | Cg6-Pg1-Ps110 | Cg6-Pg1-Ps111 | Cg6-Pg1-Ps112 |
| Cg6-Pg1-Ps113 | Cg6-Pg1-Ps114 | Cg6-Pg1-Ps115 | Cg6-Pg1-Ps116 |
| Cg6-Pg1-Ps117 | Cg6-Pg1-Ps118 | Cg6-Pg1-Ps119 | Cg6-Pg1-Ps120 |
| Cg6-Pg1-Ps121 | Cg6-Pg1-Ps122 | Cg6-Pg1-Ps123 | Cg6-Pg1-Ps124 |
| Cg6-Pg1-Ps125 | Cg6-Pg1-Ps126 | Cg6-Pg1-Ps127 | Cg6-Pg1-Ps128 |
| Cg6-Pg1-Ps129 | Cg6-Pg1-Ps130 | Cg6-Pg1-Ps131 | Cg6-Pg1-Ps132 |
| Cg6-Pg1-Ps133 | Cg6-Pg1-Ps134 | Cg6-Pg1-Ps135 | Cg6-Pg1-Ps136 |
| Cg6-Pg1-Ps137 | Cg6-Pg1-Ps138 | Cg6-Pg1-Ps139 | Cg6-Pg1-Ps140 |
| Cg6-Pg1-Ps141 | Cg6-Pg1-Ps142 | Cg6-Pg1-Ps143 | Cg6-Pg1-Ps144 |
| Cg6-Pg1-Ps145 | Cg6-Pg1-Ps146 | Cg6-Pg1-Ps147 | Cg6-Pg1-Ps148 |
| Cg6-Pg1-Ps149 | Cg6-Pg1-Ps150 | Cg6-Pg1-Ps151 | Cg6-Pg1-Ps152 |
| Cg6-Pg1-Ps153 | Cg6-Pg1-Ps154 | Cg6-Pg1-Ps155 | Cg6-Pg1-Ps156 |
| Cg6-Pg1-Ps157 | Cg6-Pg1-Ps158 | Cg6-Pg1-Ps159 | Cg6-Pg1-Ps160 |
| Cg6-Pg1-Ps161 | Cg6-Pg1-Ps162 | Cg6-Pg1-Ps163 | Cg6-Pg1-Ps164 |
| Cg6-Pg1-Ps165 | Cg6-Pg1-Ps166 | Cg6-Pg1-Ps167 | Cg6-Pg1-Ps168 |
| Cg6-Pg1-Ps169 | Cg6-Pg1-Ps170 | Cg6-Pg1-Ps171 | Cg6-Pg1-Ps172 |
| Cg6-Pg1-Ps173 | Cg6-Pg1-Ps174 | Cg6-Pg1-Ps175 | Cg6-Pg1-Ps176 |
| Cg6-Pg1-Ps177 | Cg6-Pg1-Ps178 | Cg6-Pg1-Ps179 | Cg6-Pg1-Ps180 |
| Cg6-Pg1-Ps181 | Cg6-Pg1-Ps182 | Cg6-Pg1-Ps183 | Cg6-Pg1-Ps184 |
| Cg6-Pg1-Ps185 | Cg6-Pg1-Ps186 | Cg6-Pg1-Ps187 | Cg6-Pg1-Ps188 |
| Cg6-Pg1-Ps189 | Cg6-Pg1-Ps190 | Cg6-Pg1-Ps191 | Cg6-Pg1-Ps192 |
| Cg6-Pg1-Ps193 | Cg6-Pg1-Ps194 | Cg6-Pg1-Ps195 | Cg6-Pg1-Ps196 |
| Cg6-Pg1-Ps197 | Cg6-Pg1-Ps198 | Cg6-Pg1-Ps199 | Cg6-Pg1-Ps200 |
| Cg6-Pg1-Ps201 | Cg6-Pg1-Ps202 | Cg6-Pg1-Ps203 | Cg6-Pg1-Ps204 |
| Cg6-Pg1-Ps205 | Cg6-Pg1-Ps206 | Cg6-Pg1-Ps207 | Cg6-Pg1-Ps208 |
| Cg6-Pg1-Ps209 | Cg6-Pg1-Ps210 | Cg6-Pg1-Ps211 | Cg6-Pg1-Ps212 |
| Cg6-Pg1-Ps213 | Cg6-Pg1-Ps214 | Cg6-Pg1-Ps215 | Cg6-Pg1-Ps216 |
| Cg6-Pg1-Ps217 | Cg6-Pg1-Ps218 | Cg6-Pg1-Ps219 | Cg6-Pg1-Ps220 |
| Cg6-Pg1-Ps221 | Cg6-Pg1-Ps222 | Cg6-Pg1-Ps223 | Cg6-Pg1-Ps224 |
| Cg6-Pg1-Ps225 | Cg6-Pg1-Ps226 | Cg6-Pg1-Ps227 | Cg6-Pg1-Ps228 |
| Cg6-Pg1-Ps229 | Cg6-Pg1-Ps230 | Cg6-Pg1-Ps231 | Cg6-Pg1-Ps232 |
| Cg6-Pg1-Ps233 | Cg6-Pg1-Ps234 | Cg6-Pg1-Ps235 | Cg6-Pg1-Ps236 |
| Cg6-Pg1-Ps237 | Cg6-Pg1-Ps238 | Cg6-Pg1-Ps239 | Cg6-Pg1-Ps240 |
| Cg6-Pg1-Ps241 | Cg6-Pg1-Ps242 | Cg6-Pg1-Ps243 | |
| Cg7-Pg1-Ps1 | Cg7-Pg1-Ps2 | Cg7-Pg1-Ps3 | Cg7-Pg1-Ps4 |
| Cg7-Pg1-Ps5 | Cg7-Pg1-Ps6 | Cg7-Pg1-Ps7 | Cg7-Pg1-Ps8 |
| Cg7-Pg1-Ps9 | Cg7-Pg1-Ps10 | Cg7-Pg1-Ps11 | Cg7-Pg1-Ps12 |
| Cg7-Pg1-Ps13 | Cg7-Pg1-Ps14 | Cg7-Pg1-Ps15 | Cg7-Pg1-Ps16 |
| Cg7-Pg1-Ps17 | Cg7-Pg1-Ps18 | Cg7-Pg1-Ps19 | Cg7-Pg1-Ps20 |
| Cg7-Pg1-Ps21 | Cg7-Pg1-Ps22 | Cg7-Pg1-Ps23 | Cg7-Pg1-Ps24 |
| Cg7-Pg1-Ps25 | Cg7-Pg1-Ps26 | Cg7-Pg1-Ps27 | Cg7-Pg1-Ps28 |
| Cg7-Pg1-Ps29 | Cg7-Pg1-Ps30 | Cg7-Pg1-Ps31 | Cg7-Pg1-Ps32 |
| Cg7-Pg1-Ps33 | Cg7-Pg1-Ps34 | Cg7-Pg1-Ps35 | Cg7-Pg1-Ps36 |
| Cg7-Pg1-Ps37 | Cg7-Pg1-Ps38 | Cg7-Pg1-Ps39 | Cg7-Pg1-Ps40 |
| Cg7-Pg1-Ps41 | Cg7-Pg1-Ps42 | Cg7-Pg1-Ps43 | Cg7-Pg1-Ps44 |
| Cg7-Pg1-Ps45 | Cg7-Pg1-Ps46 | Cg7-Pg1-Ps47 | Cg7-Pg1-Ps48 |
| Cg7-Pg1-Ps49 | Cg7-Pg1-Ps50 | Cg7-Pg1-Ps51 | Cg7-Pg1-Ps52 |
| Cg7-Pg1-Ps53 | Cg7-Pg1-Ps54 | Cg7-Pg1-Ps55 | Cg7-Pg1-Ps56 |
| Cg7-Pg1-Ps57 | Cg7-Pg1-Ps58 | Cg7-Pg1-Ps59 | Cg7-Pg1-Ps60 |
| Cg7-Pg1-Ps61 | Cg7-Pg1-Ps62 | Cg7-Pg1-Ps63 | Cg7-Pg1-Ps64 |
| Cg7-Pg1-Ps65 | Cg7-Pg1-Ps66 | Cg7-Pg1-Ps67 | Cg7-Pg1-Ps68 |
| Cg7-Pg1-Ps69 | Cg7-Pg1-Ps70 | Cg7-Pg1-Ps71 | Cg7-Pg1-Ps72 |
| Cg7-Pg1-Ps73 | Cg7-Pg1-Ps74 | Cg7-Pg1-Ps75 | Cg7-Pg1-Ps76 |
| Cg7-Pg1-Ps77 | Cg7-Pg1-Ps78 | Cg7-Pg1-Ps79 | Cg7-Pg1-Ps80 |
| Cg7-Pg1-Ps81 | Cg7-Pg1-Ps82 | Cg7-Pg1-Ps83 | Cg7-Pg1-Ps84 |
| Cg7-Pg1-Ps85 | Cg7-Pg1-Ps86 | Cg7-Pg1-Ps87 | Cg7-Pg1-Ps88 |
| Cg7-Pg1-Ps89 | Cg7-Pg1-Ps90 | Cg7-Pg1-Ps91 | Cg7-Pg1-Ps92 |
| Cg7-Pg1-Ps93 | Cg7-Pg1-Ps94 | Cg7-Pg1-Ps95 | Cg7-Pg1-Ps96 |
| Cg7-Pg1-Ps97 | Cg7-Pg1-Ps98 | Cg7-Pg1-Ps99 | Cg7-Pg1-Ps100 |
| Cg7-Pg1-Ps101 | Cg7-Pg1-Ps102 | Cg7-Pg1-Ps103 | Cg7-Pg1-Ps104 |
| Cg7-Pg1-Ps105 | Cg7-Pg1-Ps106 | Cg7-Pg1-Ps107 | Cg7-Pg1-Ps108 |
| Cg7-Pg1-Ps109 | Cg7-Pg1-Ps110 | Cg7-Pg1-Ps111 | Cg7-Pg1-Ps112 |
| Cg7-Pg1-Ps113 | Cg7-Pg1-Ps114 | Cg7-Pg1-Ps115 | Cg7-Pg1-Ps116 |
| Cg7-Pg1-Ps117 | Cg7-Pg1-Ps118 | Cg7-Pg1-Ps119 | Cg7-Pg1-Ps120 |
| Cg7-Pg1-Ps121 | Cg7-Pg1-Ps122 | Cg7-Pg1-Ps123 | Cg7-Pg1-Ps124 |
| Cg7-Pg1-Ps125 | Cg7-Pg1-Ps126 | Cg7-Pg1-Ps127 | Cg7-Pg1-Ps128 |
| Cg7-Pg1-Ps129 | Cg7-Pg1-Ps130 | Cg7-Pg1-Ps131 | Cg7-Pg1-Ps132 |
| Cg7-Pg1-Ps133 | Cg7-Pg1-Ps134 | Cg7-Pg1-Ps135 | Cg7-Pg1-Ps136 |
| Cg7-Pg1-Ps137 | Cg7-Pg1-Ps138 | Cg7-Pg1-Ps139 | Cg7-Pg1-Ps140 |
| Cg7-Pg1-Ps141 | Cg7-Pg1-Ps142 | Cg7-Pg1-Ps143 | Cg7-Pg1-Ps144 |
| Cg7-Pg1-Ps145 | Cg7-Pg1-Ps146 | Cg7-Pg1-Ps147 | Cg7-Pg1-Ps148 |
| Cg7-Pg1-Ps149 | Cg7-Pg1-Ps150 | Cg7-Pg1-Ps151 | Cg7-Pg1-Ps152 |
| Cg7-Pg1-Ps153 | Cg7-Pg1-Ps154 | Cg7-Pg1-Ps155 | Cg7-Pg1-Ps156 |
| Cg7-Pg1-Ps157 | Cg7-Pg1-Ps158 | Cg7-Pg1-Ps159 | Cg7-Pg1-Ps160 |
| Cg7-Pg1-Ps161 | Cg7-Pg1-Ps162 | Cg7-Pg1-Ps163 | Cg7-Pg1-Ps164 |
| Cg7-Pg1-Ps165 | Cg7-Pg1-Ps166 | Cg7-Pg1-Ps167 | Cg7-Pg1-Ps168 |
| Cg7-Pg1-Ps169 | Cg7-Pg1-Ps170 | Cg7-Pg1-Ps171 | Cg7-Pg1-Ps172 |
| Cg7-Pg1-Ps173 | Cg7-Pg1-Ps174 | Cg7-Pg1-Ps175 | Cg7-Pg1-Ps176 |
| Cg7-Pg1-Ps177 | Cg7-Pg1-Ps178 | Cg7-Pg1-Ps179 | Cg7-Pg1-Ps180 |
| Cg7-Pg1-Ps181 | Cg7-Pg1-Ps182 | Cg7-Pg1-Ps183 | Cg7-Pg1-Ps184 |
| Cg7-Pg1-Ps185 | Cg7-Pg1-Ps186 | Cg7-Pg1-Ps187 | Cg7-Pg1-Ps188 |
| Cg7-Pg1-Ps189 | Cg7-Pg1-Ps190 | Cg7-Pg1-Ps191 | Cg7-Pg1-Ps192 |
| Cg7-Pg1-Ps193 | Cg7-Pg1-Ps194 | Cg7-Pg1-Ps195 | Cg7-Pg1-Ps196 |
| Cg7-Pg1-Ps197 | Cg7-Pg1-Ps198 | Cg7-Pg1-Ps199 | Cg7-Pg1-Ps200 |
| Cg7-Pg1-Ps201 | Cg7-Pg1-Ps202 | Cg7-Pg1-Ps203 | Cg7-Pg1-Ps204 |
| Cg7-Pg1-Ps205 | Cg7-Pg1-Ps206 | Cg7-Pg1-Ps207 | Cg7-Pg1-Ps208 |
| Cg7-Pg1-Ps209 | Cg7-Pg1-Ps210 | Cg7-Pg1-Ps211 | Cg7-Pg1-Ps212 |
| Cg7-Pg1-Ps213 | Cg7-Pg1-Ps214 | Cg7-Pg1-Ps215 | Cg7-Pg1-Ps216 |
| Cg7-Pg1-Ps217 | Cg7-Pg1-Ps218 | Cg7-Pg1-Ps219 | Cg7-Pg1-Ps220 |
| Cg7-Pg1-Ps221 | Cg7-Pg1-Ps222 | Cg7-Pg1-Ps223 | Cg7-Pg1-Ps224 |
| Cg7-Pg1-Ps225 | Cg7-Pg1-Ps226 | Cg7-Pg1-Ps227 | Cg7-Pg1-Ps228 |
| Cg7-Pg1-Ps229 | Cg7-Pg1-Ps230 | Cg7-Pg1-Ps231 | Cg7-Pg1-Ps232 |
| Cg7-Pg1-Ps233 | Cg7-Pg1-Ps234 | Cg7-Pg1-Ps235 | Cg7-Pg1-Ps236 |
| Cg7-Pg1-Ps237 | Cg7-Pg1-Ps238 | Cg7-Pg1-Ps239 | Cg7-Pg1-Ps240 |
| Cg7-Pg1-Ps241 | Cg7-Pg1-Ps242 | Cg7-Pg1-Ps243 | |
| Cg18-Pg1-Ps1 | Cg18-Pg1-Ps2 | Cg18-Pg1-Ps3 | Cg18-Pg1-Ps4 |
| Cg18-Pg1-Ps5 | Cg18-Pg1-Ps6 | Cg18-Pg1-Ps7 | Cg18-Pg1-Ps8 |
| Cg18-Pg1-Ps9 | Cg18-Pg1-Ps10 | Cg18-Pg1-Ps11 | Cg18-Pg1-Ps12 |
| Cg18-Pg1-Ps13 | Cg18-Pg1-Ps14 | Cg18-Pg1-Ps15 | Cg18-Pg1-Ps16 |
| Cg18-Pg1-Ps17 | Cg18-Pg1-Ps18 | Cg18-Pg1-Ps19 | Cg18-Pg1-Ps20 |
| Cg18-Pg1-Ps21 | Cg18-Pg1-Ps22 | Cg18-Pg1-Ps23 | Cg18-Pg1-Ps24 |
| Cg18-Pg1-Ps25 | Cg18-Pg1-Ps26 | Cg18-Pg1-Ps27 | Cg18-Pg1-Ps28 |
| Cg18-Pg1-Ps29 | Cg18-Pg1-Ps30 | Cg18-Pg1-Ps31 | Cg18-Pg1-Ps32 |
| Cg18-Pg1-Ps33 | Cg18-Pg1-Ps34 | Cg18-Pg1-Ps35 | Cg18-Pg1-Ps36 |
| Cg18-Pg1-Ps37 | Cg18-Pg1-Ps38 | Cg18-Pg1-Ps39 | Cg18-Pg1-Ps40 |
| Cg18-Pg1-Ps41 | Cg18-Pg1-Ps42 | Cg18-Pg1-Ps43 | Cg18-Pg1-Ps44 |
| Cg18-Pg1-Ps45 | Cg18-Pg1-Ps46 | Cg18-Pg1-Ps47 | Cg18-Pg1-Ps48 |
| Cg18-Pg1-Ps49 | Cg18-Pg1-Ps50 | Cg18-Pg1-Ps51 | Cg18-Pg1-Ps52 |
| Cg18-Pg1-Ps53 | Cg18-Pg1-Ps54 | Cg18-Pg1-Ps55 | Cg18-Pg1-Ps56 |
| Cg18-Pg1-Ps57 | Cg18-Pg1-Ps58 | Cg18-Pg1-Ps59 | Cg18-Pg1-Ps60 |
| Cg18-Pg1-Ps61 | Cg18-Pg1-Ps62 | Cg18-Pg1-Ps63 | Cg18-Pg1-Ps64 |
| Cg18-Pg1-Ps65 | Cg18-Pg1-Ps66 | Cg18-Pg1-Ps67 | Cg18-Pg1-Ps68 |
| Cg18-Pg1-Ps69 | Cg18-Pg1-Ps70 | Cg18-Pg1-Ps71 | Cg18-Pg1-Ps72 |
| Cg18-Pg1-Ps73 | Cg18-Pg1-Ps74 | Cg18-Pg1-Ps75 | Cg18-Pg1-Ps76 |
| Cg18-Pg1-Ps77 | Cg18-Pg1-Ps78 | Cg18-Pg1-Ps79 | Cg18-Pg1-Ps80 |
| Cg18-Pg1-Ps81 | Cg18-Pg1-Ps82 | Cg18-Pg1-Ps83 | Cg18-Pg1-Ps84 |
| Cg18-Pg1-Ps85 | Cg18-Pg1-Ps86 | Cg18-Pg1-Ps87 | Cg18-Pg1-Ps88 |
| Cg18-Pg1-Ps89 | Cg18-Pg1-Ps90 | Cg18-Pg1-Ps91 | Cg18-Pg1-Ps92 |
| Cg18-Pg1-Ps93 | Cg18-Pg1-Ps94 | Cg18-Pg1-Ps95 | Cg18-Pg1-Ps96 |
| Cg18-Pg1-Ps97 | Cg18-Pg1-Ps98 | Cg18-Pg1-Ps99 | Cg18-Pg1-Ps100 |
| Cg18-Pg1-Ps101 | Cg18-Pg1-Ps102 | Cg18-Pg1-Ps103 | Cg18-Pg1-Ps104 |
| Cg18-Pg1-Ps105 | Cg18-Pg1-Ps106 | Cg18-Pg1-Ps107 | Cg18-Pg1-Ps108 |
| Cg18-Pg1-Ps109 | Cg18-Pg1-Ps110 | Cg18-Pg1-Ps111 | Cg18-Pg1-Ps112 |
| Cg18-Pg1-Ps113 | Cg18-Pg1-Ps114 | Cg18-Pg1-Ps115 | Cg18-Pg1-Ps116 |
| Cg18-Pg1-Ps117 | Cg18-Pg1-Ps118 | Cg18-Pg1-Ps119 | Cg18-Pg1-Ps120 |
| Cg18-Pg1-Ps121 | Cg18-Pg1-Ps122 | Cg18-Pg1-Ps123 | Cg18-Pg1-Ps124 |
| Cg18-Pg1-Ps125 | Cg18-Pg1-Ps126 | Cg18-Pg1-Ps127 | Cg18-Pg1-Ps128 |
| Cg18-Pg1-Ps129 | Cg18-Pg1-Ps130 | Cg18-Pg1-Ps131 | Cg18-Pg1-Ps132 |
| Cg18-Pg1-Ps133 | Cg18-Pg1-Ps134 | Cg18-Pg1-Ps135 | Cg18-Pg1-Ps136 |
| Cg18-Pg1-Ps137 | Cg18-Pg1-Ps138 | Cg18-Pg1-Ps139 | Cg18-Pg1-Ps140 |
| Cg18-Pg1-Ps141 | Cg18-Pg1-Ps142 | Cg18-Pg1-Ps143 | Cg18-Pg1-Ps144 |
| Cg18-Pg1-Ps145 | Cg18-Pg1-Ps146 | Cg18-Pg1-Ps147 | Cg18-Pg1-Ps148 |
| Cg18-Pg1-Ps149 | Cg18-Pg1-Ps150 | Cg18-Pg1-Ps151 | Cg18-Pg1-Ps152 |
| Cg18-Pg1-Ps153 | Cg18-Pg1-Ps154 | Cg18-Pg1-Ps155 | Cg18-Pg1-Ps156 |
| Cg18-Pg1-Ps157 | Cg18-Pg1-Ps158 | Cg18-Pg1-Ps159 | Cg18-Pg1-Ps160 |
| Cg18-Pg1-Ps161 | Cg18-Pg1-Ps162 | Cg18-Pg1-Ps163 | Cg18-Pg1-Ps164 |
| Cg18-Pg1-Ps165 | Cg18-Pg1-Ps166 | Cg18-Pg1-Ps167 | Cg18-Pg1-Ps168 |
| Cg18-Pg1-Ps169 | Cg18-Pg1-Ps170 | Cg18-Pg1-Ps171 | Cg18-Pg1-Ps172 |
| Cg18-Pg1-Ps173 | Cg18-Pg1-Ps174 | Cg18-Pg1-Ps175 | Cg18-Pg1-Ps176 |
| Cg18-Pg1-Ps177 | Cg18-Pg1-Ps178 | Cg18-Pg1-Ps179 | Cg18-Pg1-Ps180 |
| Cg18-Pg1-Ps181 | Cg18-Pg1-Ps182 | Cg18-Pg1-Ps183 | Cg18-Pg1-Ps184 |
| Cg18-Pg1-Ps185 | Cg18-Pg1-Ps186 | Cg18-Pg1-Ps187 | Cg18-Pg1-Ps188 |
| Cg18-Pg1-Ps189 | Cg18-Pg1-Ps190 | Cg18-Pg1-Ps191 | Cg18-Pg1-Ps192 |
| Cg18-Pg1-Ps193 | Cg18-Pg1-Ps194 | Cg18-Pg1-Ps195 | Cg18-Pg1-Ps196 |
| Cg18-Pg1-Ps197 | Cg18-Pg1-Ps198 | Cg18-Pg1-Ps199 | Cg18-Pg1-Ps200 |
| Cg18-Pg1-Ps201 | Cg18-Pg1-Ps202 | Cg18-Pg1-Ps203 | Cg18-Pg1-Ps204 |
| Cg18-Pg1-Ps205 | Cg18-Pg1-Ps206 | Cg18-Pg1-Ps207 | Cg18-Pg1-Ps208 |
| Cg18-Pg1-Ps209 | Cg18-Pg1-Ps210 | Cg18-Pg1-Ps211 | Cg18-Pg1-Ps212 |
| Cg18-Pg1-Ps213 | Cg18-Pg1-Ps214 | Cg18-Pg1-Ps215 | Cg18-Pg1-Ps216 |
| Cg18-Pg1-Ps217 | Cg18-Pg1-Ps218 | Cg18-Pg1-Ps219 | Cg18-Pg1-Ps220 |
| Cg18-Pg1-Ps221 | Cg18-Pg1-Ps222 | Cg18-Pg1-Ps223 | Cg18-Pg1-Ps224 |
| Cg18-Pg1-Ps225 | Cg18-Pg1-Ps226 | Cg18-Pg1-Ps227 | Cg18-Pg1-Ps228 |
| Cg18-Pg1-Ps229 | Cg18-Pg1-Ps230 | Cg18-Pg1-Ps231 | Cg18-Pg1-Ps232 |
| Cg18-Pg1-Ps233 | Cg18-Pg1-Ps234 | Cg18-Pg1-Ps235 | Cg18-Pg1-Ps236 |
| Cg18-Pg1-Ps237 | Cg18-Pg1-Ps238 | Cg18-Pg1-Ps239 | Cg18-Pg1-Ps240 |
| Cg18-Pg1-Ps241 | Cg18-Pg1-Ps242 | Cg18-Pg1-Ps243 | |
| Cg21-Pg1-Ps1 | Cg21-Pg1-Ps2 | Cg21-Pg1-Ps3 | Cg21-Pg1-Ps4 |
| Cg21-Pg1-Ps5 | Cg21-Pg1-Ps6 | Cg21-Pg1-Ps7 | Cg21-Pg1-Ps8 |
| Cg21-Pg1-Ps9 | Cg21-Pg1-Ps10 | Cg21-Pg1-Ps11 | Cg21-Pg1-Ps12 |
| Cg21-Pg1-Ps13 | Cg21-Pg1-Ps14 | Cg21-Pg1-Ps15 | Cg21-Pg1-Ps16 |
| Cg21-Pg1-Ps17 | Cg21-Pg1-Ps18 | Cg21-Pg1-Ps19 | Cg21-Pg1-Ps20 |
| Cg21-Pg1-Ps21 | Cg21-Pg1-Ps22 | Cg21-Pg1-Ps23 | Cg21-Pg1-Ps24 |
| Cg21-Pg1-Ps25 | Cg21-Pg1-Ps26 | Cg21-Pg1-Ps27 | Cg21-Pg1-Ps28 |
| Cg21-Pg1-Ps29 | Cg21-Pg1-Ps30 | Cg21-Pg1-Ps31 | Cg21-Pg1-Ps32 |
| Cg21-Pg1-Ps33 | Cg21-Pg1-Ps34 | Cg21-Pg1-Ps35 | Cg21-Pg1-Ps36 |
| Cg21-Pg1-Ps37 | Cg21-Pg1-Ps38 | Cg21-Pg1-Ps39 | Cg21-Pg1-Ps40 |
| Cg21-Pg1-Ps41 | Cg21-Pg1-Ps42 | Cg21-Pg1-Ps43 | Cg21-Pg1-Ps44 |
| Cg21-Pg1-Ps45 | Cg21-Pg1-Ps46 | Cg21-Pg1-Ps47 | Cg21-Pg1-Ps48 |
| Cg21-Pg1-Ps49 | Cg21-Pg1-Ps50 | Cg21-Pg1-Ps51 | Cg21-Pg1-Ps52 |
| Cg21-Pg1-Ps53 | Cg21-Pg1-Ps54 | Cg21-Pg1-Ps55 | Cg21-Pg1-Ps56 |
| Cg21-Pg1-Ps57 | Cg21-Pg1-Ps58 | Cg21-Pg1-Ps59 | Cg21-Pg1-Ps60 |
| Cg21-Pg1-Ps61 | Cg21-Pg1-Ps62 | Cg21-Pg1-Ps63 | Cg21-Pg1-Ps64 |
| Cg21-Pg1-Ps65 | Cg21-Pg1-Ps66 | Cg21-Pg1-Ps67 | Cg21-Pg1-Ps68 |
| Cg21-Pg1-Ps69 | Cg21-Pg1-Ps70 | Cg21-Pg1-Ps71 | Cg21-Pg1-Ps72 |
| Cg21-Pg1-Ps73 | Cg21-Pg1-Ps74 | Cg21-Pg1-Ps75 | Cg21-Pg1-Ps76 |
| Cg21-Pg1-Ps77 | Cg21-Pg1-Ps78 | Cg21-Pg1-Ps79 | Cg21-Pg1-Ps80 |
| Cg21-Pg1-Ps81 | Cg21-Pg1-Ps82 | Cg21-Pg1-Ps83 | Cg21-Pg1-Ps84 |
| Cg21-Pg1-Ps85 | Cg21-Pg1-Ps86 | Cg21-Pg1-Ps87 | Cg21-Pg1-Ps88 |
| Cg21-Pg1-Ps89 | Cg21-Pg1-Ps90 | Cg21-Pg1-Ps91 | Cg21-Pg1-Ps92 |
| Cg21-Pg1-Ps93 | Cg21-Pg1-Ps94 | Cg21-Pg1-Ps95 | Cg21-Pg1-Ps96 |
| Cg21-Pg1-Ps97 | Cg21-Pg1-Ps98 | Cg21-Pg1-Ps99 | Cg21-Pg1-Ps100 |
| Cg21-Pg1-Ps101 | Cg21-Pg1-Ps102 | Cg21-Pg1-Ps103 | Cg21-Pg1-Ps104 |
| Cg21-Pg1-Ps105 | Cg21-Pg1-Ps106 | Cg21-Pg1-Ps107 | Cg21-Pg1-Ps108 |
| Cg21-Pg1-Ps109 | Cg21-Pg1-Ps110 | Cg21-Pg1-Ps111 | Cg21-Pg1-Ps112 |
| Cg21-Pg1-Ps113 | Cg21-Pg1-Ps114 | Cg21-Pg1-Ps115 | Cg21-Pg1-Ps116 |
| Cg21-Pg1-Ps117 | Cg21-Pg1-Ps118 | Cg21-Pg1-Ps119 | Cg21-Pg1-Ps120 |
| Cg21-Pg1-Ps121 | Cg21-Pg1-Ps122 | Cg21-Pg1-Ps123 | Cg21-Pg1-Ps124 |
| Cg21-Pg1-Ps125 | Cg21-Pg1-Ps126 | Cg21-Pg1-Ps127 | Cg21-Pg1-Ps128 |
| Cg21-Pg1-Ps129 | Cg21-Pg1-Ps130 | Cg21-Pg1-Ps131 | Cg21-Pg1-Ps132 |
| Cg21-Pg1-Ps133 | Cg21-Pg1-Ps134 | Cg21-Pg1-Ps135 | Cg21-Pg1-Ps136 |
| Cg21-Pg1-Ps137 | Cg21-Pg1-Ps138 | Cg21-Pg1-Ps139 | Cg21-Pg1-Ps140 |
| Cg21-Pg1-Ps141 | Cg21-Pg1-Ps142 | Cg21-Pg1-Ps143 | Cg21-Pg1-Ps144 |
| Cg21-Pg1-Ps145 | Cg21-Pg1-Ps146 | Cg21-Pg1-Ps147 | Cg21-Pg1-Ps148 |
| Cg21-Pg1-Ps149 | Cg21-Pg1-Ps150 | Cg21-Pg1-Ps151 | Cg21-Pg1-Ps152 |
| Cg21-Pg1-Ps153 | Cg21-Pg1-Ps154 | Cg21-Pg1-Ps155 | Cg21-Pg1-Ps156 |
| Cg21-Pg1-Ps157 | Cg21-Pg1-Ps158 | Cg21-Pg1-Ps159 | Cg21-Pg1-Ps160 |
| Cg21-Pg1-Ps161 | Cg21-Pg1-Ps162 | Cg21-Pg1-Ps163 | Cg21-Pg1-Ps164 |
| Cg21-Pg1-Ps165 | Cg21-Pg1-Ps166 | Cg21-Pg1-Ps167 | Cg21-Pg1-Ps168 |
| Cg21-Pg1-Ps169 | Cg21-Pg1-Ps170 | Cg21-Pg1-Ps171 | Cg21-Pg1-Ps172 |
| Cg21-Pg1-Ps173 | Cg21-Pg1-Ps174 | Cg21-Pg1-Ps175 | Cg21-Pg1-Ps176 |
| Cg21-Pg1-Ps177 | Cg21-Pg1-Ps178 | Cg21-Pg1-Ps179 | Cg21-Pg1-Ps180 |
| Cg21-Pg1-Ps181 | Cg21-Pg1-Ps182 | Cg21-Pg1-Ps183 | Cg21-Pg1-Ps184 |
| Cg21-Pg1-Ps185 | Cg21-Pg1-Ps186 | Cg21-Pg1-Ps187 | Cg21-Pg1-Ps188 |
| Cg21-Pg1-Ps189 | Cg21-Pg1-Ps190 | Cg21-Pg1-Ps191 | Cg21-Pg1-Ps192 |
| Cg21-Pg1-Ps193 | Cg21-Pg1-Ps194 | Cg21-Pg1-Ps195 | Cg21-Pg1-Ps196 |
| Cg21-Pg1-Ps197 | Cg21-Pg1-Ps198 | Cg21-Pg1-Ps199 | Cg21-Pg1-Ps200 |
| Cg21-Pg1-Ps201 | Cg21-Pg1-Ps202 | Cg21-Pg1-Ps203 | Cg21-Pg1-Ps204 |
| Cg21-Pg1-Ps205 | Cg21-Pg1-Ps206 | Cg21-Pg1-Ps207 | Cg21-Pg1-Ps208 |
| Cg21-Pg1-Ps209 | Cg21-Pg1-Ps210 | Cg21-Pg1-Ps211 | Cg21-Pg1-Ps212 |
| Cg21-Pg1-Ps213 | Cg21-Pg1-Ps214 | Cg21-Pg1-Ps215 | Cg21-Pg1-Ps216 |
| Cg21-Pg1-Ps217 | Cg21-Pg1-Ps218 | Cg21-Pg1-Ps219 | Cg21-Pg1-Ps220 |
| Cg21-Pg1-Ps221 | Cg21-Pg1-Ps222 | Cg21-Pg1-Ps223 | Cg21-Pg1-Ps224 |
| Cg21-Pg1-Ps225 | Cg21-Pg1-Ps226 | Cg21-Pg1-Ps227 | Cg21-Pg1-Ps228 |
| Cg21-Pg1-Ps229 | Cg21-Pg1-Ps230 | Cg21-Pg1-Ps231 | Cg21-Pg1-Ps232 |
| Cg21-Pg1-Ps233 | Cg21-Pg1-Ps234 | Cg21-Pg1-Ps235 | Cg21-Pg1-Ps236 |
| Cg21-Pg1-Ps237 | Cg21-Pg1-Ps238 | Cg21-Pg1-Ps239 | Cg21-Pg1-Ps240 |
| Cg21-Pg1-Ps241 | Cg21-Pg1-Ps242 | Cg21-Pg1-Ps243 | |
| Cg22-Pg1-Ps1 | Cg22-Pg1-Ps2 | Cg22-Pg1-Ps3 | Cg22-Pg1-Ps4 |
| Cg22-Pg1-Ps5 | Cg22-Pg1-Ps6 | Cg22-Pg1-Ps7 | Cg22-Pg1-Ps8 |
| Cg22-Pg1-Ps9 | Cg22-Pg1-Ps10 | Cg22-Pg1-Ps11 | Cg22-Pg1-Ps12 |
| Cg22-Pg1-Ps13 | Cg22-Pg1-Ps14 | Cg22-Pg1-Ps15 | Cg22-Pg1-Ps16 |
| Cg22-Pg1-Ps17 | Cg22-Pg1-Ps18 | Cg22-Pg1-Ps19 | Cg22-Pg1-Ps20 |
| Cg22-Pg1-Ps21 | Cg22-Pg1-Ps22 | Cg22-Pg1-Ps23 | Cg22-Pg1-Ps24 |
| Cg22-Pg1-Ps25 | Cg22-Pg1-Ps26 | Cg22-Pg1-Ps27 | Cg22-Pg1-Ps28 |
| Cg22-Pg1-Ps29 | Cg22-Pg1-Ps30 | Cg22-Pg1-Ps31 | Cg22-Pg1-Ps32 |
| Cg22-Pg1-Ps33 | Cg22-Pg1-Ps34 | Cg22-Pg1-Ps35 | Cg22-Pg1-Ps36 |
| Cg22-Pg1-Ps37 | Cg22-Pg1-Ps38 | Cg22-Pg1-Ps39 | Cg22-Pg1-Ps40 |
| Cg22-Pg1-Ps41 | Cg22-Pg1-Ps42 | Cg22-Pg1-Ps43 | Cg22-Pg1-Ps44 |
| Cg22-Pg1-Ps45 | Cg22-Pg1-Ps46 | Cg22-Pg1-Ps47 | Cg22-Pg1-Ps48 |
| Cg22-Pg1-Ps49 | Cg22-Pg1-Ps50 | Cg22-Pg1-Ps51 | Cg22-Pg1-Ps52 |
| Cg22-Pg1-Ps53 | Cg22-Pg1-Ps54 | Cg22-Pg1-Ps55 | Cg22-Pg1-Ps56 |
| Cg22-Pg1-Ps57 | Cg22-Pg1-Ps58 | Cg22-Pg1-Ps59 | Cg22-Pg1-Ps60 |
| Cg22-Pg1-Ps61 | Cg22-Pg1-Ps62 | Cg22-Pg1-Ps63 | Cg22-Pg1-Ps64 |
| Cg22-Pg1-Ps65 | Cg22-Pg1-Ps66 | Cg22-Pg1-Ps67 | Cg22-Pg1-Ps68 |
| Cg22-Pg1-Ps69 | Cg22-Pg1-Ps70 | Cg22-Pg1-Ps71 | Cg22-Pg1-Ps72 |
| Cg22-Pg1-Ps73 | Cg22-Pg1-Ps74 | Cg22-Pg1-Ps75 | Cg22-Pg1-Ps76 |
| Cg22-Pg1-Ps77 | Cg22-Pg1-Ps78 | Cg22-Pg1-Ps79 | Cg22-Pg1-Ps80 |
| Cg22-Pg1-Ps81 | Cg22-Pg1-Ps82 | Cg22-Pg1-Ps83 | Cg22-Pg1-Ps84 |
| Cg22-Pg1-Ps85 | Cg22-Pg1-Ps86 | Cg22-Pg1-Ps87 | Cg22-Pg1-Ps88 |
| Cg22-Pg1-Ps89 | Cg22-Pg1-Ps90 | Cg22-Pg1-Ps91 | Cg22-Pg1-Ps92 |
| Cg22-Pg1-Ps93 | Cg22-Pg1-Ps94 | Cg22-Pg1-Ps95 | Cg22-Pg1-Ps96 |
| Cg22-Pg1-Ps97 | Cg22-Pg1-Ps98 | Cg22-Pg1-Ps99 | Cg22-Pg1-Ps100 |
| Cg22-Pg1-Ps101 | Cg22-Pg1-Ps102 | Cg22-Pg1-Ps103 | Cg22-Pg1-Ps104 |
| Cg22-Pg1-Ps105 | Cg22-Pg1-Ps106 | Cg22-Pg1-Ps107 | Cg22-Pg1-Ps108 |
| Cg22-Pg1-Ps109 | Cg22-Pg1-Ps110 | Cg22-Pg1-Ps111 | Cg22-Pg1-Ps112 |
| Cg22-Pg1-Ps113 | Cg22-Pg1-Ps114 | Cg22-Pg1-Ps115 | Cg22-Pg1-Ps116 |
| Cg22-Pg1-Ps117 | Cg22-Pg1-Ps118 | Cg22-Pg1-Ps119 | Cg22-Pg1-Ps120 |
| Cg22-Pg1-Ps121 | Cg22-Pg1-Ps122 | Cg22-Pg1-Ps123 | Cg22-Pg1-Ps124 |
| Cg22-Pg1-Ps125 | Cg22-Pg1-Ps126 | Cg22-Pg1-Ps127 | Cg22-Pg1-Ps128 |
| Cg22-Pg1-Ps129 | Cg22-Pg1-Ps130 | Cg22-Pg1-Ps131 | Cg22-Pg1-Ps132 |
| Cg22-Pg1-Ps133 | Cg22-Pg1-Ps134 | Cg22-Pg1-Ps135 | Cg22-Pg1-Ps136 |
| Cg22-Pg1-Ps137 | Cg22-Pg1-Ps138 | Cg22-Pg1-Ps139 | Cg22-Pg1-Ps140 |
| Cg22-Pg1-Ps141 | Cg22-Pg1-Ps142 | Cg22-Pg1-Ps143 | Cg22-Pg1-Ps144 |
| Cg22-Pg1-Ps145 | Cg22-Pg1-Ps146 | Cg22-Pg1-Ps147 | Cg22-Pg1-Ps148 |
| Cg22-Pg1-Ps149 | Cg22-Pg1-Ps150 | Cg22-Pg1-Ps151 | Cg22-Pg1-Ps152 |
| Cg22-Pg1-Ps153 | Cg22-Pg1-Ps154 | Cg22-Pg1-Ps155 | Cg22-Pg1-Ps156 |
| Cg22-Pg1-Ps157 | Cg22-Pg1-Ps158 | Cg22-Pg1-Ps159 | Cg22-Pg1-Ps160 |
| Cg22-Pg1-Ps161 | Cg22-Pg1-Ps162 | Cg22-Pg1-Ps163 | Cg22-Pg1-Ps164 |
| Cg22-Pg1-Ps165 | Cg22-Pg1-Ps166 | Cg22-Pg1-Ps167 | Cg22-Pg1-Ps168 |
| Cg22-Pg1-Ps169 | Cg22-Pg1-Ps170 | Cg22-Pg1-Ps171 | Cg22-Pg1-Ps172 |
| Cg22-Pg1-Ps173 | Cg22-Pg1-Ps174 | Cg22-Pg1-Ps175 | Cg22-Pg1-Ps176 |
| Cg22-Pg1-Ps177 | Cg22-Pg1-Ps178 | Cg22-Pg1-Ps179 | Cg22-Pg1-Ps180 |
| Cg22-Pg1-Ps181 | Cg22-Pg1-Ps182 | Cg22-Pg1-Ps183 | Cg22-Pg1-Ps184 |
| Cg22-Pg1-Ps185 | Cg22-Pg1-Ps186 | Cg22-Pg1-Ps187 | Cg22-Pg1-Ps188 |
| Cg22-Pg1-Ps189 | Cg22-Pg1-Ps190 | Cg22-Pg1-Ps191 | Cg22-Pg1-Ps192 |
| Cg22-Pg1-Ps193 | Cg22-Pg1-Ps194 | Cg22-Pg1-Ps195 | Cg22-Pg1-Ps196 |
| Cg22-Pg1-Ps197 | Cg22-Pg1-Ps198 | Cg22-Pg1-Ps199 | Cg22-Pg1-Ps200 |
| Cg22-Pg1-Ps201 | Cg22-Pg1-Ps202 | Cg22-Pg1-Ps203 | Cg22-Pg1-Ps204 |
| Cg22-Pg1-Ps205 | Cg22-Pg1-Ps206 | Cg22-Pg1-Ps207 | Cg22-Pg1-Ps208 |
| Cg22-Pg1-Ps209 | Cg22-Pg1-Ps210 | Cg22-Pg1-Ps211 | Cg22-Pg1-Ps212 |
| Cg22-Pg1-Ps213 | Cg22-Pg1-Ps214 | Cg22-Pg1-Ps215 | Cg22-Pg1-Ps216 |
| Cg22-Pg1-Ps217 | Cg22-Pg1-Ps218 | Cg22-Pg1-Ps219 | Cg22-Pg1-Ps220 |
| Cg22-Pg1-Ps221 | Cg22-Pg1-Ps222 | Cg22-Pg1-Ps223 | Cg22-Pg1-Ps224 |
| Cg22-Pg1-Ps225 | Cg22-Pg1-Ps226 | Cg22-Pg1-Ps227 | Cg22-Pg1-Ps228 |
| Cg22-Pg1-Ps229 | Cg22-Pg1-Ps230 | Cg22-Pg1-Ps231 | Cg22-Pg1-Ps232 |
| Cg22-Pg1-Ps233 | Cg22-Pg1-Ps234 | Cg22-Pg1-Ps235 | Cg22-Pg1-Ps236 |
| Cg22-Pg1-Ps237 | Cg22-Pg1-Ps238 | Cg22-Pg1-Ps239 | Cg22-Pg1-Ps240 |
| Cg22-Pg1-Ps241 | Cg22-Pg1-Ps242 | Cg22-Pg1-Ps243 | |
| Cg23-Pg1-Ps1 | Cg23-Pg1-Ps2 | Cg23-Pg1-Ps3 | Cg23-Pg1-Ps4 |
| Cg23-Pg1-Ps5 | Cg23-Pg1-Ps6 | Cg23-Pg1-Ps7 | Cg23-Pg1-Ps8 |
| Cg23-Pg1-Ps9 | Cg23-Pg1-Ps10 | Cg23-Pg1-Ps11 | Cg23-Pg1-Ps12 |
| Cg23-Pg1-Ps13 | Cg23-Pg1-Ps14 | Cg23-Pg1-Ps15 | Cg23-Pg1-Ps16 |
| Cg23-Pg1-Ps17 | Cg23-Pg1-Ps18 | Cg23-Pg1-Ps19 | Cg23-Pg1-Ps20 |
| Cg23-Pg1-Ps21 | Cg23-Pg1-Ps22 | Cg23-Pg1-Ps23 | Cg23-Pg1-Ps24 |
| Cg23-Pg1-Ps25 | Cg23-Pg1-Ps26 | Cg23-Pg1-Ps27 | Cg23-Pg1-Ps28 |
| Cg23-Pg1-Ps29 | Cg23-Pg1-Ps30 | Cg23-Pg1-Ps31 | Cg23-Pg1-Ps32 |
| Cg23-Pg1-Ps33 | Cg23-Pg1-Ps34 | Cg23-Pg1-Ps35 | Cg23-Pg1-Ps36 |
| Cg23-Pg1-Ps37 | Cg23-Pg1-Ps38 | Cg23-Pg1-Ps39 | Cg23-Pg1-Ps40 |
| Cg23-Pg1-Ps41 | Cg23-Pg1-Ps42 | Cg23-Pg1-Ps43 | Cg23-Pg1-Ps44 |
| Cg23-Pg1-Ps45 | Cg23-Pg1-Ps46 | Cg23-Pg1-Ps47 | Cg23-Pg1-Ps48 |
| Cg23-Pg1-Ps49 | Cg23-Pg1-Ps50 | Cg23-Pg1-Ps51 | Cg23-Pg1-Ps52 |
| Cg23-Pg1-Ps53 | Cg23-Pg1-Ps54 | Cg23-Pg1-Ps55 | Cg23-Pg1-Ps56 |
| Cg23-Pg1-Ps57 | Cg23-Pg1-Ps58 | Cg23-Pg1-Ps59 | Cg23-Pg1-Ps60 |
| Cg23-Pg1-Ps61 | Cg23-Pg1-Ps62 | Cg23-Pg1-Ps63 | Cg23-Pg1-Ps64 |
| Cg23-Pg1-Ps65 | Cg23-Pg1-Ps66 | Cg23-Pg1-Ps67 | Cg23-Pg1-Ps68 |
| Cg23-Pg1-Ps69 | Cg23-Pg1-Ps70 | Cg23-Pg1-Ps71 | Cg23-Pg1-Ps72 |
| Cg23-Pg1-Ps73 | Cg23-Pg1-Ps74 | Cg23-Pg1-Ps75 | Cg23-Pg1-Ps76 |
| Cg23-Pg1-Ps77 | Cg23-Pg1-Ps78 | Cg23-Pg1-Ps79 | Cg23-Pg1-Ps80 |
| Cg23-Pg1-Ps81 | Cg23-Pg1-Ps82 | Cg23-Pg1-Ps83 | Cg23-Pg1-Ps84 |
| Cg23-Pg1-Ps85 | Cg23-Pg1-Ps86 | Cg23-Pg1-Ps87 | Cg23-Pg1-Ps88 |
| Cg23-Pg1-Ps89 | Cg23-Pg1-Ps90 | Cg23-Pg1-Ps91 | Cg23-Pg1-Ps92 |
| Cg23-Pg1-Ps93 | Cg23-Pg1-Ps94 | Cg23-Pg1-Ps95 | Cg23-Pg1-Ps96 |
| Cg23-Pg1-Ps97 | Cg23-Pg1-Ps98 | Cg23-Pg1-Ps99 | Cg23-Pg1-Ps100 |
| Cg23-Pg1-Ps101 | Cg23-Pg1-Ps102 | Cg23-Pg1-Ps103 | Cg23-Pg1-Ps104 |
| Cg23-Pg1-Ps105 | Cg23-Pg1-Ps106 | Cg23-Pg1-Ps107 | Cg23-Pg1-Ps108 |
| Cg23-Pg1-Ps109 | Cg23-Pg1-Ps110 | Cg23-Pg1-Ps111 | Cg23-Pg1-Ps112 |
| Cg23-Pg1-Ps113 | Cg23-Pg1-Ps114 | Cg23-Pg1-Ps115 | Cg23-Pg1-Ps116 |
| Cg23-Pg1-Ps117 | Cg23-Pg1-Ps118 | Cg23-Pg1-Ps119 | Cg23-Pg1-Ps120 |
| Cg23-Pg1-Ps121 | Cg23-Pg1-Ps122 | Cg23-Pg1-Ps123 | Cg23-Pg1-Ps124 |
| Cg23-Pg1-Ps125 | Cg23-Pg1-Ps126 | Cg23-Pg1-Ps127 | Cg23-Pg1-Ps128 |
| Cg23-Pg1-Ps129 | Cg23-Pg1-Ps130 | Cg23-Pg1-Ps131 | Cg23-Pg1-Ps132 |
| Cg23-Pg1-Ps133 | Cg23-Pg1-Ps134 | Cg23-Pg1-Ps135 | Cg23-Pg1-Ps136 |
| Cg23-Pg1-Ps137 | Cg23-Pg1-Ps138 | Cg23-Pg1-Ps139 | Cg23-Pg1-Ps140 |
| Cg23-Pg1-Ps141 | Cg23-Pg1-Ps142 | Cg23-Pg1-Ps143 | Cg23-Pg1-Ps144 |
| Cg23-Pg1-Ps145 | Cg23-Pg1-Ps146 | Cg23-Pg1-Ps147 | Cg23-Pg1-Ps148 |
| Cg23-Pg1-Ps149 | Cg23-Pg1-Ps150 | Cg23-Pg1-Ps151 | Cg23-Pg1-Ps152 |
| Cg23-Pg1-Ps153 | Cg23-Pg1-Ps154 | Cg23-Pg1-Ps155 | Cg23-Pg1-Ps156 |
| Cg23-Pg1-Ps157 | Cg23-Pg1-Ps158 | Cg23-Pg1-Ps159 | Cg23-Pg1-Ps160 |
| Cg23-Pg1-Ps161 | Cg23-Pg1-Ps162 | Cg23-Pg1-Ps163 | Cg23-Pg1-Ps164 |
| Cg23-Pg1-Ps165 | Cg23-Pg1-Ps166 | Cg23-Pg1-Ps167 | Cg23-Pg1-Ps168 |
| Cg23-Pg1-Ps169 | Cg23-Pg1-Ps170 | Cg23-Pg1-Ps171 | Cg23-Pg1-Ps172 |
| Cg23-Pg1-Ps173 | Cg23-Pg1-Ps174 | Cg23-Pg1-Ps175 | Cg23-Pg1-Ps176 |
| Cg23-Pg1-Ps177 | Cg23-Pg1-Ps178 | Cg23-Pg1-Ps179 | Cg23-Pg1-Ps180 |
| Cg23-Pg1-Ps181 | Cg23-Pg1-Ps182 | Cg23-Pg1-Ps183 | Cg23-Pg1-Ps184 |
| Cg23-Pg1-Ps185 | Cg23-Pg1-Ps186 | Cg23-Pg1-Ps187 | Cg23-Pg1-Ps188 |
| Cg23-Pg1-Ps189 | Cg23-Pg1-Ps190 | Cg23-Pg1-Ps191 | Cg23-Pg1-Ps192 |
| Cg23-Pg1-Ps193 | Cg23-Pg1-Ps194 | Cg23-Pg1-Ps195 | Cg23-Pg1-Ps196 |
| Cg23-Pg1-Ps197 | Cg23-Pg1-Ps198 | Cg23-Pg1-Ps199 | Cg23-Pg1-Ps200 |
| Cg23-Pg1-Ps201 | Cg23-Pg1-Ps202 | Cg23-Pg1-Ps203 | Cg23-Pg1-Ps204 |
| Cg23-Pg1-Ps205 | Cg23-Pg1-Ps206 | Cg23-Pg1-Ps207 | Cg23-Pg1-Ps208 |
| Cg23-Pg1-Ps209 | Cg23-Pg1-Ps210 | Cg23-Pg1-Ps211 | Cg23-Pg1-Ps212 |
| Cg23-Pg1-Ps213 | Cg23-Pg1-Ps214 | Cg23-Pg1-Ps215 | Cg23-Pg1-Ps216 |
| Cg23-Pg1-Ps217 | Cg23-Pg1-Ps218 | Cg23-Pg1-Ps219 | Cg23-Pg1-Ps220 |
| Cg23-Pg1-Ps221 | Cg23-Pg1-Ps222 | Cg23-Pg1-Ps223 | Cg23-Pg1-Ps224 |
| Cg23-Pg1-Ps225 | Cg23-Pg1-Ps226 | Cg23-Pg1-Ps227 | Cg23-Pg1-Ps228 |
| Cg23-Pg1-Ps229 | Cg23-Pg1-Ps230 | Cg23-Pg1-Ps231 | Cg23-Pg1-Ps232 |
| Cg23-Pg1-Ps233 | Cg23-Pg1-Ps234 | Cg23-Pg1-Ps235 | Cg23-Pg1-Ps236 |
| Cg23-Pg1-Ps237 | Cg23-Pg1-Ps238 | Cg23-Pg1-Ps239 | Cg23-Pg1-Ps240 |
| Cg23-Pg1-Ps241 | Cg23-Pg1-Ps242 | Cg23-Pg1-Ps243 | |
| Cg24-Pg1-Ps1 | Cg24-Pg1-Ps2 | Cg24-Pg1-Ps3 | Cg24-Pg1-Ps4 |
| Cg24-Pg1-Ps5 | Cg24-Pg1-Ps6 | Cg24-Pg1-Ps7 | Cg24-Pg1-Ps8 |
| Cg24-Pg1-Ps9 | Cg24-Pg1-Ps10 | Cg24-Pg1-Ps11 | Cg24-Pg1-Ps12 |
| Cg24-Pg1-Ps13 | Cg24-Pg1-Ps14 | Cg24-Pg1-Ps15 | Cg24-Pg1-Ps16 |
| Cg24-Pg1-Ps17 | Cg24-Pg1-Ps18 | Cg24-Pg1-Ps19 | Cg24-Pg1-Ps20 |
| Cg24-Pg1-Ps21 | Cg24-Pg1-Ps22 | Cg24-Pg1-Ps23 | Cg24-Pg1-Ps24 |
| Cg24-Pg1-Ps25 | Cg24-Pg1-Ps26 | Cg24-Pg1-Ps27 | Cg24-Pg1-Ps28 |
| Cg24-Pg1-Ps29 | Cg24-Pg1-Ps30 | Cg24-Pg1-Ps31 | Cg24-Pg1-Ps32 |
| Cg24-Pg1-Ps33 | Cg24-Pg1-Ps34 | Cg24-Pg1-Ps35 | Cg24-Pg1-Ps36 |
| Cg24-Pg1-Ps37 | Cg24-Pg1-Ps38 | Cg24-Pg1-Ps39 | Cg24-Pg1-Ps40 |
| Cg24-Pg1-Ps41 | Cg24-Pg1-Ps42 | Cg24-Pg1-Ps43 | Cg24-Pg1-Ps44 |
| Cg24-Pg1-Ps45 | Cg24-Pg1-Ps46 | Cg24-Pg1-Ps47 | Cg24-Pg1-Ps48 |
| Cg24-Pg1-Ps49 | Cg24-Pg1-Ps50 | Cg24-Pg1-Ps51 | Cg24-Pg1-Ps52 |
| Cg24-Pg1-Ps53 | Cg24-Pg1-Ps54 | Cg24-Pg1-Ps55 | Cg24-Pg1-Ps56 |
| Cg24-Pg1-Ps57 | Cg24-Pg1-Ps58 | Cg24-Pg1-Ps59 | Cg24-Pg1-Ps60 |
| Cg24-Pg1-Ps61 | Cg24-Pg1-Ps62 | Cg24-Pg1-Ps63 | Cg24-Pg1-Ps64 |
| Cg24-Pg1-Ps65 | Cg24-Pg1-Ps66 | Cg24-Pg1-Ps67 | Cg24-Pg1-Ps68 |
| Cg24-Pg1-Ps69 | Cg24-Pg1-Ps70 | Cg24-Pg1-Ps71 | Cg24-Pg1-Ps72 |
| Cg24-Pg1-Ps73 | Cg24-Pg1-Ps74 | Cg24-Pg1-Ps75 | Cg24-Pg1-Ps76 |
| Cg24-Pg1-Ps77 | Cg24-Pg1-Ps78 | Cg24-Pg1-Ps79 | Cg24-Pg1-Ps80 |
| Cg24-Pg1-Ps81 | Cg24-Pg1-Ps82 | Cg24-Pg1-Ps83 | Cg24-Pg1-Ps84 |
| Cg24-Pg1-Ps85 | Cg24-Pg1-Ps86 | Cg24-Pg1-Ps87 | Cg24-Pg1-Ps88 |
| Cg24-Pg1-Ps89 | Cg24-Pg1-Ps90 | Cg24-Pg1-Ps91 | Cg24-Pg1-Ps92 |
| Cg24-Pg1-Ps93 | Cg24-Pg1-Ps94 | Cg24-Pg1-Ps95 | Cg24-Pg1-Ps96 |
| Cg24-Pg1-Ps97 | Cg24-Pg1-Ps98 | Cg24-Pg1-Ps99 | Cg24-Pg1-Ps100 |
| Cg24-Pg1-Ps101 | Cg24-Pg1-Ps102 | Cg24-Pg1-Ps103 | Cg24-Pg1-Ps104 |
| Cg24-Pg1-Ps105 | Cg24-Pg1-Ps106 | Cg24-Pg1-Ps107 | Cg24-Pg1-Ps108 |
| Cg24-Pg1-Ps109 | Cg24-Pg1-Ps110 | Cg24-Pg1-Ps111 | Cg24-Pg1-Ps112 |
| Cg24-Pg1-Ps113 | Cg24-Pg1-Ps114 | Cg24-Pg1-Ps115 | Cg24-Pg1-Ps116 |
| Cg24-Pg1-Ps117 | Cg24-Pg1-Ps118 | Cg24-Pg1-Ps119 | Cg24-Pg1-Ps120 |
| Cg24-Pg1-Ps121 | Cg24-Pg1-Ps122 | Cg24-Pg1-Ps123 | Cg24-Pg1-Ps124 |
| Cg24-Pg1-Ps125 | Cg24-Pg1-Ps126 | Cg24-Pg1-Ps127 | Cg24-Pg1-Ps128 |
| Cg24-Pg1-Ps129 | Cg24-Pg1-Ps130 | Cg24-Pg1-Ps131 | Cg24-Pg1-Ps132 |
| Cg24-Pg1-Ps133 | Cg24-Pg1-Ps134 | Cg24-Pg1-Ps135 | Cg24-Pg1-Ps136 |
| Cg24-Pg1-Ps137 | Cg24-Pg1-Ps138 | Cg24-Pg1-Ps139 | Cg24-Pg1-Ps140 |
| Cg24-Pg1-Ps141 | Cg24-Pg1-Ps142 | Cg24-Pg1-Ps143 | Cg24-Pg1-Ps144 |
| Cg24-Pg1-Ps145 | Cg24-Pg1-Ps146 | Cg24-Pg1-Ps147 | Cg24-Pg1-Ps148 |
| Cg24-Pg1-Ps149 | Cg24-Pg1-Ps150 | Cg24-Pg1-Ps151 | Cg24-Pg1-Ps152 |
| Cg24-Pg1-Ps153 | Cg24-Pg1-Ps154 | Cg24-Pg1-Ps155 | Cg24-Pg1-Ps156 |
| Cg24-Pg1-Ps157 | Cg24-Pg1-Ps158 | Cg24-Pg1-Ps159 | Cg24-Pg1-Ps160 |
| Cg24-Pg1-Ps161 | Cg24-Pg1-Ps162 | Cg24-Pg1-Ps163 | Cg24-Pg1-Ps164 |
| Cg24-Pg1-Ps165 | Cg24-Pg1-Ps166 | Cg24-Pg1-Ps167 | Cg24-Pg1-Ps168 |
| Cg24-Pg1-Ps169 | Cg24-Pg1-Ps170 | Cg24-Pg1-Ps171 | Cg24-Pg1-Ps172 |
| Cg24-Pg1-Ps173 | Cg24-Pg1-Ps174 | Cg24-Pg1-Ps175 | Cg24-Pg1-Ps176 |
| Cg24-Pg1-Ps177 | Cg24-Pg1-Ps178 | Cg24-Pg1-Ps179 | Cg24-Pg1-Ps180 |
| Cg24-Pg1-Ps181 | Cg24-Pg1-Ps182 | Cg24-Pg1-Ps183 | Cg24-Pg1-Ps184 |
| Cg24-Pg1-Ps185 | Cg24-Pg1-Ps186 | Cg24-Pg1-Ps187 | Cg24-Pg1-Ps188 |
| Cg24-Pg1-Ps189 | Cg24-Pg1-Ps190 | Cg24-Pg1-Ps191 | Cg24-Pg1-Ps192 |
| Cg24-Pg1-Ps193 | Cg24-Pg1-Ps194 | Cg24-Pg1-Ps195 | Cg24-Pg1-Ps196 |
| Cg24-Pg1-Ps197 | Cg24-Pg1-Ps198 | Cg24-Pg1-Ps199 | Cg24-Pg1-Ps200 |
| Cg24-Pg1-Ps201 | Cg24-Pg1-Ps202 | Cg24-Pg1-Ps203 | Cg24-Pg1-Ps204 |
| Cg24-Pg1-Ps205 | Cg24-Pg1-Ps206 | Cg24-Pg1-Ps207 | Cg24-Pg1-Ps208 |
| Cg24-Pg1-Ps209 | Cg24-Pg1-Ps210 | Cg24-Pg1-Ps211 | Cg24-Pg1-Ps212 |
| Cg24-Pg1-Ps213 | Cg24-Pg1-Ps214 | Cg24-Pg1-Ps215 | Cg24-Pg1-Ps216 |
| Cg24-Pg1-Ps217 | Cg24-Pg1-Ps218 | Cg24-Pg1-Ps219 | Cg24-Pg1-Ps220 |
| Cg24-Pg1-Ps221 | Cg24-Pg1-Ps222 | Cg24-Pg1-Ps223 | Cg24-Pg1-Ps224 |
| Cg24-Pg1-Ps225 | Cg24-Pg1-Ps226 | Cg24-Pg1-Ps227 | Cg24-Pg1-Ps228 |
| Cg24-Pg1-Ps229 | Cg24-Pg1-Ps230 | Cg24-Pg1-Ps231 | Cg24-Pg1-Ps232 |
| Cg24-Pg1-Ps233 | Cg24-Pg1-Ps234 | Cg24-Pg1-Ps235 | Cg24-Pg1-Ps236 |
| Cg24-Pg1-Ps237 | Cg24-Pg1-Ps238 | Cg24-Pg1-Ps239 | Cg24-Pg1-Ps240 |
| Cg24-Pg1-Ps241 | Cg24-Pg1-Ps242 | Cg24-Pg1-Ps243 | |
| Cg25-Pg1-Ps1 | Cg25-Pg1-Ps2 | Cg25-Pg1-Ps3 | Cg25-Pg1-Ps4 |
| Cg25-Pg1-Ps5 | Cg25-Pg1-Ps6 | Cg25-Pg1-Ps7 | Cg25-Pg1-Ps8 |
| Cg25-Pg1-Ps9 | Cg25-Pg1-Ps10 | Cg25-Pg1-Ps11 | Cg25-Pg1-Ps12 |
| Cg25-Pg1-Ps13 | Cg25-Pg1-Ps14 | Cg25-Pg1-Ps15 | Cg25-Pg1-Ps16 |
| Cg25-Pg1-Ps17 | Cg25-Pg1-Ps18 | Cg25-Pg1-Ps19 | Cg25-Pg1-Ps20 |
| Cg25-Pg1-Ps21 | Cg25-Pg1-Ps22 | Cg25-Pg1-Ps23 | Cg25-Pg1-Ps24 |
| Cg25-Pg1-Ps25 | Cg25-Pg1-Ps26 | Cg25-Pg1-Ps27 | Cg25-Pg1-Ps28 |
| Cg25-Pg1-Ps29 | Cg25-Pg1-Ps30 | Cg25-Pg1-Ps31 | Cg25-Pg1-Ps32 |
| Cg25-Pg1-Ps33 | Cg25-Pg1-Ps34 | Cg25-Pg1-Ps35 | Cg25-Pg1-Ps36 |
| Cg25-Pg1-Ps37 | Cg25-Pg1-Ps38 | Cg25-Pg1-Ps39 | Cg25-Pg1-Ps40 |
| Cg25-Pg1-Ps41 | Cg25-Pg1-Ps42 | Cg25-Pg1-Ps43 | Cg25-Pg1-Ps44 |
| Cg25-Pg1-Ps45 | Cg25-Pg1-Ps46 | Cg25-Pg1-Ps47 | Cg25-Pg1-Ps48 |
| Cg25-Pg1-Ps49 | Cg25-Pg1-Ps50 | Cg25-Pg1-Ps51 | Cg25-Pg1-Ps52 |
| Cg25-Pg1-Ps53 | Cg25-Pg1-Ps54 | Cg25-Pg1-Ps55 | Cg25-Pg1-Ps56 |
| Cg25-Pg1-Ps57 | Cg25-Pg1-Ps58 | Cg25-Pg1-Ps59 | Cg25-Pg1-Ps60 |
| Cg25-Pg1-Ps61 | Cg25-Pg1-Ps62 | Cg25-Pg1-Ps63 | Cg25-Pg1-Ps64 |
| Cg25-Pg1-Ps65 | Cg25-Pg1-Ps66 | Cg25-Pg1-Ps67 | Cg25-Pg1-Ps68 |
| Cg25-Pg1-Ps69 | Cg25-Pg1-Ps70 | Cg25-Pg1-Ps71 | Cg25-Pg1-Ps72 |
| Cg25-Pg1-Ps73 | Cg25-Pg1-Ps74 | Cg25-Pg1-Ps75 | Cg25-Pg1-Ps76 |
| Cg25-Pg1-Ps77 | Cg25-Pg1-Ps78 | Cg25-Pg1-Ps79 | Cg25-Pg1-Ps80 |
| Cg25-Pg1-Ps81 | Cg25-Pg1-Ps82 | Cg25-Pg1-Ps83 | Cg25-Pg1-Ps84 |
| Cg25-Pg1-Ps85 | Cg25-Pg1-Ps86 | Cg25-Pg1-Ps87 | Cg25-Pg1-Ps88 |
| Cg25-Pg1-Ps89 | Cg25-Pg1-Ps90 | Cg25-Pg1-Ps91 | Cg25-Pg1-Ps92 |
| Cg25-Pg1-Ps93 | Cg25-Pg1-Ps94 | Cg25-Pg1-Ps95 | Cg25-Pg1-Ps96 |
| Cg25-Pg1-Ps97 | Cg25-Pg1-Ps98 | Cg25-Pg1-Ps99 | Cg25-Pg1-Ps100 |
| Cg25-Pg1-Ps101 | Cg25-Pg1-Ps102 | Cg25-Pg1-Ps103 | Cg25-Pg1-Ps104 |
| Cg25-Pg1-Ps105 | Cg25-Pg1-Ps106 | Cg25-Pg1-Ps107 | Cg25-Pg1-Ps108 |
| Cg25-Pg1-Ps109 | Cg25-Pg1-Ps110 | Cg25-Pg1-Ps111 | Cg25-Pg1-Ps112 |
| Cg25-Pg1-Ps113 | Cg25-Pg1-Ps114 | Cg25-Pg1-Ps115 | Cg25-Pg1-Ps116 |
| Cg25-Pg1-Ps117 | Cg25-Pg1-Ps118 | Cg25-Pg1-Ps119 | Cg25-Pg1-Ps120 |
| Cg25-Pg1-Ps121 | Cg25-Pg1-Ps122 | Cg25-Pg1-Ps123 | Cg25-Pg1-Ps124 |
| Cg25-Pg1-Ps125 | Cg25-Pg1-Ps126 | Cg25-Pg1-Ps127 | Cg25-Pg1-Ps128 |
| Cg25-Pg1-Ps129 | Cg25-Pg1-Ps130 | Cg25-Pg1-Ps131 | Cg25-Pg1-Ps132 |
| Cg25-Pg1-Ps133 | Cg25-Pg1-Ps134 | Cg25-Pg1-Ps135 | Cg25-Pg1-Ps136 |
| Cg25-Pg1-Ps137 | Cg25-Pg1-Ps138 | Cg25-Pg1-Ps139 | Cg25-Pg1-Ps140 |
| Cg25-Pg1-Ps141 | Cg25-Pg1-Ps142 | Cg25-Pg1-Ps143 | Cg25-Pg1-Ps144 |
| Cg25-Pg1-Ps145 | Cg25-Pg1-Ps146 | Cg25-Pg1-Ps147 | Cg25-Pg1-Ps148 |
| Cg25-Pg1-Ps149 | Cg25-Pg1-Ps150 | Cg25-Pg1-Ps151 | Cg25-Pg1-Ps152 |
| Cg25-Pg1-Ps153 | Cg25-Pg1-Ps154 | Cg25-Pg1-Ps155 | Cg25-Pg1-Ps156 |
| Cg25-Pg1-Ps157 | Cg25-Pg1-Ps158 | Cg25-Pg1-Ps159 | Cg25-Pg1-Ps160 |
| Cg25-Pg1-Ps161 | Cg25-Pg1-Ps162 | Cg25-Pg1-Ps163 | Cg25-Pg1-Ps164 |
| Cg25-Pg1-Ps165 | Cg25-Pg1-Ps166 | Cg25-Pg1-Ps167 | Cg25-Pg1-Ps168 |
| Cg25-Pg1-Ps169 | Cg25-Pg1-Ps170 | Cg25-Pg1-Ps171 | Cg25-Pg1-Ps172 |
| Cg25-Pg1-Ps173 | Cg25-Pg1-Ps174 | Cg25-Pg1-Ps175 | Cg25-Pg1-Ps176 |
| Cg25-Pg1-Ps177 | Cg25-Pg1-Ps178 | Cg25-Pg1-Ps179 | Cg25-Pg1-Ps180 |
| Cg25-Pg1-Ps181 | Cg25-Pg1-Ps182 | Cg25-Pg1-Ps183 | Cg25-Pg1-Ps184 |
| Cg25-Pg1-Ps185 | Cg25-Pg1-Ps186 | Cg25-Pg1-Ps187 | Cg25-Pg1-Ps188 |
| Cg25-Pg1-Ps189 | Cg25-Pg1-Ps190 | Cg25-Pg1-Ps191 | Cg25-Pg1-Ps192 |
| Cg25-Pg1-Ps193 | Cg25-Pg1-Ps194 | Cg25-Pg1-Ps195 | Cg25-Pg1-Ps196 |
| Cg25-Pg1-Ps197 | Cg25-Pg1-Ps198 | Cg25-Pg1-Ps199 | Cg25-Pg1-Ps200 |
| Cg25-Pg1-Ps201 | Cg25-Pg1-Ps202 | Cg25-Pg1-Ps203 | Cg25-Pg1-Ps204 |
| Cg25-Pg1-Ps205 | Cg25-Pg1-Ps206 | Cg25-Pg1-Ps207 | Cg25-Pg1-Ps208 |
| Cg25-Pg1-Ps209 | Cg25-Pg1-Ps210 | Cg25-Pg1-Ps211 | Cg25-Pg1-Ps212 |
| Cg25-Pg1-Ps213 | Cg25-Pg1-Ps214 | Cg25-Pg1-Ps215 | Cg25-Pg1-Ps216 |
| Cg25-Pg1-Ps217 | Cg25-Pg1-Ps218 | Cg25-Pg1-Ps219 | Cg25-Pg1-Ps220 |
| Cg25-Pg1-Ps221 | Cg25-Pg1-Ps222 | Cg25-Pg1-Ps223 | Cg25-Pg1-Ps224 |
| Cg25-Pg1-Ps225 | Cg25-Pg1-Ps226 | Cg25-Pg1-Ps227 | Cg25-Pg1-Ps228 |
| Cg25-Pg1-Ps229 | Cg25-Pg1-Ps230 | Cg25-Pg1-Ps231 | Cg25-Pg1-Ps232 |
| Cg25-Pg1-Ps233 | Cg25-Pg1-Ps234 | Cg25-Pg1-Ps235 | Cg25-Pg1-Ps236 |
| Cg25-Pg1-Ps237 | Cg25-Pg1-Ps238 | Cg25-Pg1-Ps239 | Cg25-Pg1-Ps240 |
| Cg25-Pg1-Ps241 | Cg25-Pg1-Ps242 | Cg25-Pg1-Ps243 | |
| Cg26-Pg1-Ps1 | Cg26-Pg1-Ps2 | Cg26-Pg1-Ps3 | Cg26-Pg1-Ps4 |
| Cg26-Pg1-Ps5 | Cg26-Pg1-Ps6 | Cg26-Pg1-Ps7 | Cg26-Pg1-Ps8 |
| Cg26-Pg1-Ps9 | Cg26-Pg1-Ps10 | Cg26-Pg1-Ps11 | Cg26-Pg1-Ps12 |
| Cg26-Pg1-Ps13 | Cg26-Pg1-Ps14 | Cg26-Pg1-Ps15 | Cg26-Pg1-Ps16 |
| Cg26-Pg1-Ps17 | Cg26-Pg1-Ps18 | Cg26-Pg1-Ps19 | Cg26-Pg1-Ps20 |
| Cg26-Pg1-Ps21 | Cg26-Pg1-Ps22 | Cg26-Pg1-Ps23 | Cg26-Pg1-Ps24 |
| Cg26-Pg1-Ps25 | Cg26-Pg1-Ps26 | Cg26-Pg1-Ps27 | Cg26-Pg1-Ps28 |
| Cg26-Pg1-Ps29 | Cg26-Pg1-Ps30 | Cg26-Pg1-Ps31 | Cg26-Pg1-Ps32 |
| Cg26-Pg1-Ps33 | Cg26-Pg1-Ps34 | Cg26-Pg1-Ps35 | Cg26-Pg1-Ps36 |
| Cg26-Pg1-Ps37 | Cg26-Pg1-Ps38 | Cg26-Pg1-Ps39 | Cg26-Pg1-Ps40 |
| Cg26-Pg1-Ps41 | Cg26-Pg1-Ps42 | Cg26-Pg1-Ps43 | Cg26-Pg1-Ps44 |
| Cg26-Pg1-Ps45 | Cg26-Pg1-Ps46 | Cg26-Pg1-Ps47 | Cg26-Pg1-Ps48 |
| Cg26-Pg1-Ps49 | Cg26-Pg1-Ps50 | Cg26-Pg1-Ps51 | Cg26-Pg1-Ps52 |
| Cg26-Pg1-Ps53 | Cg26-Pg1-Ps54 | Cg26-Pg1-Ps55 | Cg26-Pg1-Ps56 |
| Cg26-Pg1-Ps57 | Cg26-Pg1-Ps58 | Cg26-Pg1-Ps59 | Cg26-Pg1-Ps60 |
| Cg26-Pg1-Ps61 | Cg26-Pg1-Ps62 | Cg26-Pg1-Ps63 | Cg26-Pg1-Ps64 |
| Cg26-Pg1-Ps65 | Cg26-Pg1-Ps66 | Cg26-Pg1-Ps67 | Cg26-Pg1-Ps68 |
| Cg26-Pg1-Ps69 | Cg26-Pg1-Ps70 | Cg26-Pg1-Ps71 | Cg26-Pg1-Ps72 |
| Cg26-Pg1-Ps73 | Cg26-Pg1-Ps74 | Cg26-Pg1-Ps75 | Cg26-Pg1-Ps76 |
| Cg26-Pg1-Ps77 | Cg26-Pg1-Ps78 | Cg26-Pg1-Ps79 | Cg26-Pg1-Ps80 |
| Cg26-Pg1-Ps81 | Cg26-Pg1-Ps82 | Cg26-Pg1-Ps83 | Cg26-Pg1-Ps84 |
| Cg26-Pg1-Ps85 | Cg26-Pg1-Ps86 | Cg26-Pg1-Ps87 | Cg26-Pg1-Ps88 |
| Cg26-Pg1-Ps89 | Cg26-Pg1-Ps90 | Cg26-Pg1-Ps91 | Cg26-Pg1-Ps92 |
| Cg26-Pg1-Ps93 | Cg26-Pg1-Ps94 | Cg26-Pg1-Ps95 | Cg26-Pg1-Ps96 |
| Cg26-Pg1-Ps97 | Cg26-Pg1-Ps98 | Cg26-Pg1-Ps99 | Cg26-Pg1-Ps100 |
| Cg26-Pg1-Ps101 | Cg26-Pg1-Ps102 | Cg26-Pg1-Ps103 | Cg26-Pg1-Ps104 |
| Cg26-Pg1-Ps105 | Cg26-Pg1-Ps106 | Cg26-Pg1-Ps107 | Cg26-Pg1-Ps108 |
| Cg26-Pg1-Ps109 | Cg26-Pg1-Ps110 | Cg26-Pg1-Ps111 | Cg26-Pg1-Ps112 |
| Cg26-Pg1-Ps113 | Cg26-Pg1-Ps114 | Cg26-Pg1-Ps115 | Cg26-Pg1-Ps116 |
| Cg26-Pg1-Ps117 | Cg26-Pg1-Ps118 | Cg26-Pg1-Ps119 | Cg26-Pg1-Ps120 |
| Cg26-Pg1-Ps121 | Cg26-Pg1-Ps122 | Cg26-Pg1-Ps123 | Cg26-Pg1-Ps124 |
| Cg26-Pg1-Ps125 | Cg26-Pg1-Ps126 | Cg26-Pg1-Ps127 | Cg26-Pg1-Ps128 |
| Cg26-Pg1-Ps129 | Cg26-Pg1-Ps130 | Cg26-Pg1-Ps131 | Cg26-Pg1-Ps132 |
| Cg26-Pg1-Ps133 | Cg26-Pg1-Ps134 | Cg26-Pg1-Ps135 | Cg26-Pg1-Ps136 |
| Cg26-Pg1-Ps137 | Cg26-Pg1-Ps138 | Cg26-Pg1-Ps139 | Cg26-Pg1-Ps140 |
| Cg26-Pg1-Ps141 | Cg26-Pg1-Ps142 | Cg26-Pg1-Ps143 | Cg26-Pg1-Ps144 |
| Cg26-Pg1-Ps145 | Cg26-Pg1-Ps146 | Cg26-Pg1-Ps147 | Cg26-Pg1-Ps148 |
| Cg26-Pg1-Ps149 | Cg26-Pg1-Ps150 | Cg26-Pg1-Ps151 | Cg26-Pg1-Ps152 |
| Cg26-Pg1-Ps153 | Cg26-Pg1-Ps154 | Cg26-Pg1-Ps155 | Cg26-Pg1-Ps156 |
| Cg26-Pg1-Ps157 | Cg26-Pg1-Ps158 | Cg26-Pg1-Ps159 | Cg26-Pg1-Ps160 |
| Cg26-Pg1-Ps161 | Cg26-Pg1-Ps162 | Cg26-Pg1-Ps163 | Cg26-Pg1-Ps164 |
| Cg26-Pg1-Ps165 | Cg26-Pg1-Ps166 | Cg26-Pg1-Ps167 | Cg26-Pg1-Ps168 |
| Cg26-Pg1-Ps169 | Cg26-Pg1-Ps170 | Cg26-Pg1-Ps171 | Cg26-Pg1-Ps172 |
| Cg26-Pg1-Ps173 | Cg26-Pg1-Ps174 | Cg26-Pg1-Ps175 | Cg26-Pg1-Ps176 |
| Cg26-Pg1-Ps177 | Cg26-Pg1-Ps178 | Cg26-Pg1-Ps179 | Cg26-Pg1-Ps180 |
| Cg26-Pg1-Ps181 | Cg26-Pg1-Ps182 | Cg26-Pg1-Ps183 | Cg26-Pg1-Ps184 |
| Cg26-Pg1-Ps185 | Cg26-Pg1-Ps186 | Cg26-Pg1-Ps187 | Cg26-Pg1-Ps188 |
| Cg26-Pg1-Ps189 | Cg26-Pg1-Ps190 | Cg26-Pg1-Ps191 | Cg26-Pg1-Ps192 |
| Cg26-Pg1-Ps193 | Cg26-Pg1-Ps194 | Cg26-Pg1-Ps195 | Cg26-Pg1-Ps196 |
| Cg26-Pg1-Ps197 | Cg26-Pg1-Ps198 | Cg26-Pg1-Ps199 | Cg26-Pg1-Ps200 |
| Cg26-Pg1-Ps201 | Cg26-Pg1-Ps202 | Cg26-Pg1-Ps203 | Cg26-Pg1-Ps204 |
| Cg26-Pg1-Ps205 | Cg26-Pg1-Ps206 | Cg26-Pg1-Ps207 | Cg26-Pg1-Ps208 |
| Cg26-Pg1-Ps209 | Cg26-Pg1-Ps210 | Cg26-Pg1-Ps211 | Cg26-Pg1-Ps212 |
| Cg26-Pg1-Ps213 | Cg26-Pg1-Ps214 | Cg26-Pg1-Ps215 | Cg26-Pg1-Ps216 |
| Cg26-Pg1-Ps217 | Cg26-Pg1-Ps218 | Cg26-Pg1-Ps219 | Cg26-Pg1-Ps220 |
| Cg26-Pg1-Ps221 | Cg26-Pg1-Ps222 | Cg26-Pg1-Ps223 | Cg26-Pg1-Ps224 |
| Cg26-Pg1-Ps225 | Cg26-Pg1-Ps226 | Cg26-Pg1-Ps227 | Cg26-Pg1-Ps228 |
| Cg26-Pg1-Ps229 | Cg26-Pg1-Ps230 | Cg26-Pg1-Ps231 | Cg26-Pg1-Ps232 |
| Cg26-Pg1-Ps233 | Cg26-Pg1-Ps234 | Cg26-Pg1-Ps235 | Cg26-Pg1-Ps236 |
| Cg26-Pg1-Ps237 | Cg26-Pg1-Ps238 | Cg26-Pg1-Ps239 | Cg26-Pg1-Ps240 |
| Cg26-Pg1-Ps241 | Cg26-Pg1-Ps242 | Cg26-Pg1-Ps243 | |
| Cg27-Pg1-Ps1 | Cg27-Pg1-Ps2 | Cg27-Pg1-Ps3 | Cg27-Pg1-Ps4 |
| Cg27-Pg1-Ps5 | Cg27-Pg1-Ps6 | Cg27-Pg1-Ps7 | Cg27-Pg1-Ps8 |
| Cg27-Pg1-Ps9 | Cg27-Pg1-Ps10 | Cg27-Pg1-Ps11 | Cg27-Pg1-Ps12 |
| Cg27-Pg1-Ps13 | Cg27-Pg1-Ps14 | Cg27-Pg1-Ps15 | Cg27-Pg1-Ps16 |
| Cg27-Pg1-Ps17 | Cg27-Pg1-Ps18 | Cg27-Pg1-Ps19 | Cg27-Pg1-Ps20 |
| Cg27-Pg1-Ps21 | Cg27-Pg1-Ps22 | Cg27-Pg1-Ps23 | Cg27-Pg1-Ps24 |
| Cg27-Pg1-Ps25 | Cg27-Pg1-Ps26 | Cg27-Pg1-Ps27 | Cg27-Pg1-Ps28 |
| Cg27-Pg1-Ps29 | Cg27-Pg1-Ps30 | Cg27-Pg1-Ps31 | Cg27-Pg1-Ps32 |
| Cg27-Pg1-Ps33 | Cg27-Pg1-Ps34 | Cg27-Pg1-Ps35 | Cg27-Pg1-Ps36 |
| Cg27-Pg1-Ps37 | Cg27-Pg1-Ps38 | Cg27-Pg1-Ps39 | Cg27-Pg1-Ps40 |
| Cg27-Pg1-Ps41 | Cg27-Pg1-Ps42 | Cg27-Pg1-Ps43 | Cg27-Pg1-Ps44 |
| Cg27-Pg1-Ps45 | Cg27-Pg1-Ps46 | Cg27-Pg1-Ps47 | Cg27-Pg1-Ps48 |
| Cg27-Pg1-Ps49 | Cg27-Pg1-Ps50 | Cg27-Pg1-Ps51 | Cg27-Pg1-Ps52 |
| Cg27-Pg1-Ps53 | Cg27-Pg1-Ps54 | Cg27-Pg1-Ps55 | Cg27-Pg1-Ps56 |
| Cg27-Pg1-Ps57 | Cg27-Pg1-Ps58 | Cg27-Pg1-Ps59 | Cg27-Pg1-Ps60 |
| Cg27-Pg1-Ps61 | Cg27-Pg1-Ps62 | Cg27-Pg1-Ps63 | Cg27-Pg1-Ps64 |
| Cg27-Pg1-Ps65 | Cg27-Pg1-Ps66 | Cg27-Pg1-Ps67 | Cg27-Pg1-Ps68 |
| Cg27-Pg1-Ps69 | Cg27-Pg1-Ps70 | Cg27-Pg1-Ps71 | Cg27-Pg1-Ps72 |
| Cg27-Pg1-Ps73 | Cg27-Pg1-Ps74 | Cg27-Pg1-Ps75 | Cg27-Pg1-Ps76 |
| Cg27-Pg1-Ps77 | Cg27-Pg1-Ps78 | Cg27-Pg1-Ps79 | Cg27-Pg1-Ps80 |
| Cg27-Pg1-Ps81 | Cg27-Pg1-Ps82 | Cg27-Pg1-Ps83 | Cg27-Pg1-Ps84 |
| Cg27-Pg1-Ps85 | Cg27-Pg1-Ps86 | Cg27-Pg1-Ps87 | Cg27-Pg1-Ps88 |
| Cg27-Pg1-Ps89 | Cg27-Pg1-Ps90 | Cg27-Pg1-Ps91 | Cg27-Pg1-Ps92 |
| Cg27-Pg1-Ps93 | Cg27-Pg1-Ps94 | Cg27-Pg1-Ps95 | Cg27-Pg1-Ps96 |
| Cg27-Pg1-Ps97 | Cg27-Pg1-Ps98 | Cg27-Pg1-Ps99 | Cg27-Pg1-Ps100 |
| Cg27-Pg1-Ps101 | Cg27-Pg1-Ps102 | Cg27-Pg1-Ps103 | Cg27-Pg1-Ps104 |
| Cg27-Pg1-Ps105 | Cg27-Pg1-Ps106 | Cg27-Pg1-Ps107 | Cg27-Pg1-Ps108 |
| Cg27-Pg1-Ps109 | Cg27-Pg1-Ps110 | Cg27-Pg1-Ps111 | Cg27-Pg1-Ps112 |
| Cg27-Pg1-Ps113 | Cg27-Pg1-Ps114 | Cg27-Pg1-Ps115 | Cg27-Pg1-Ps116 |
| Cg27-Pg1-Ps117 | Cg27-Pg1-Ps118 | Cg27-Pg1-Ps119 | Cg27-Pg1-Ps120 |
| Cg27-Pg1-Ps121 | Cg27-Pg1-Ps122 | Cg27-Pg1-Ps123 | Cg27-Pg1-Ps124 |
| Cg27-Pg1-Ps125 | Cg27-Pg1-Ps126 | Cg27-Pg1-Ps127 | Cg27-Pg1-Ps128 |
| Cg27-Pg1-Ps129 | Cg27-Pg1-Ps130 | Cg27-Pg1-Ps131 | Cg27-Pg1-Ps132 |
| Cg27-Pg1-Ps133 | Cg27-Pg1-Ps134 | Cg27-Pg1-Ps135 | Cg27-Pg1-Ps136 |
| Cg27-Pg1-Ps137 | Cg27-Pg1-Ps138 | Cg27-Pg1-Ps139 | Cg27-Pg1-Ps140 |
| Cg27-Pg1-Ps141 | Cg27-Pg1-Ps142 | Cg27-Pg1-Ps143 | Cg27-Pg1-Ps144 |
| Cg27-Pg1-Ps145 | Cg27-Pg1-Ps146 | Cg27-Pg1-Ps147 | Cg27-Pg1-Ps148 |
| Cg27-Pg1-Ps149 | Cg27-Pg1-Ps150 | Cg27-Pg1-Ps151 | Cg27-Pg1-Ps152 |
| Cg27-Pg1-Ps153 | Cg27-Pg1-Ps154 | Cg27-Pg1-Ps155 | Cg27-Pg1-Ps156 |
| Cg27-Pg1-Ps157 | Cg27-Pg1-Ps158 | Cg27-Pg1-Ps159 | Cg27-Pg1-Ps160 |
| Cg27-Pg1-Ps161 | Cg27-Pg1-Ps162 | Cg27-Pg1-Ps163 | Cg27-Pg1-Ps164 |
| Cg27-Pg1-Ps165 | Cg27-Pg1-Ps166 | Cg27-Pg1-Ps167 | Cg27-Pg1-Ps168 |
| Cg27-Pg1-Ps169 | Cg27-Pg1-Ps170 | Cg27-Pg1-Ps171 | Cg27-Pg1-Ps172 |
| Cg27-Pg1-Ps173 | Cg27-Pg1-Ps174 | Cg27-Pg1-Ps175 | Cg27-Pg1-Ps176 |
| Cg27-Pg1-Ps177 | Cg27-Pg1-Ps178 | Cg27-Pg1-Ps179 | Cg27-Pg1-Ps180 |
| Cg27-Pg1-Ps181 | Cg27-Pg1-Ps182 | Cg27-Pg1-Ps183 | Cg27-Pg1-Ps184 |
| Cg27-Pg1-Ps185 | Cg27-Pg1-Ps186 | Cg27-Pg1-Ps187 | Cg27-Pg1-Ps188 |
| Cg27-Pg1-Ps189 | Cg27-Pg1-Ps190 | Cg27-Pg1-Ps191 | Cg27-Pg1-Ps192 |
| Cg27-Pg1-Ps193 | Cg27-Pg1-Ps194 | Cg27-Pg1-Ps195 | Cg27-Pg1-Ps196 |
| Cg27-Pg1-Ps197 | Cg27-Pg1-Ps198 | Cg27-Pg1-Ps199 | Cg27-Pg1-Ps200 |
| Cg27-Pg1-Ps201 | Cg27-Pg1-Ps202 | Cg27-Pg1-Ps203 | Cg27-Pg1-Ps204 |
| Cg27-Pg1-Ps205 | Cg27-Pg1-Ps206 | Cg27-Pg1-Ps207 | Cg27-Pg1-Ps208 |
| Cg27-Pg1-Ps209 | Cg27-Pg1-Ps210 | Cg27-Pg1-Ps211 | Cg27-Pg1-Ps212 |
| Cg27-Pg1-Ps213 | Cg27-Pg1-Ps214 | Cg27-Pg1-Ps215 | Cg27-Pg1-Ps216 |
| Cg27-Pg1-Ps217 | Cg27-Pg1-Ps218 | Cg27-Pg1-Ps219 | Cg27-Pg1-Ps220 |
| Cg27-Pg1-Ps221 | Cg27-Pg1-Ps222 | Cg27-Pg1-Ps223 | Cg27-Pg1-Ps224 |
| Cg27-Pg1-Ps225 | Cg27-Pg1-Ps226 | Cg27-Pg1-Ps227 | Cg27-Pg1-Ps228 |
| Cg27-Pg1-Ps229 | Cg27-Pg1-Ps230 | Cg27-Pg1-Ps231 | Cg27-Pg1-Ps232 |
| Cg27-Pg1-Ps233 | Cg27-Pg1-Ps234 | Cg27-Pg1-Ps235 | Cg27-Pg1-Ps236 |
| Cg27-Pg1-Ps237 | Cg27-Pg1-Ps238 | Cg27-Pg1-Ps239 | Cg27-Pg1-Ps240 |
| Cg27-Pg1-Ps241 | Cg27-Pg1-Ps242 | Cg27-Pg1-Ps243 | |
| Cg28-Pg1-Ps1 | Cg28-Pg1-Ps2 | Cg28-Pg1-Ps3 | Cg28-Pg1-Ps4 |
| Cg28-Pg1-Ps5 | Cg28-Pg1-Ps6 | Cg28-Pg1-Ps7 | Cg28-Pg1-Ps8 |
| Cg28-Pg1-Ps9 | Cg28-Pg1-Ps10 | Cg28-Pg1-Ps11 | Cg28-Pg1-Ps12 |
| Cg28-Pg1-Ps13 | Cg28-Pg1-Ps14 | Cg28-Pg1-Ps15 | Cg28-Pg1-Ps16 |
| Cg28-Pg1-Ps17 | Cg28-Pg1-Ps18 | Cg28-Pg1-Ps19 | Cg28-Pg1-Ps20 |
| Cg28-Pg1-Ps21 | Cg28-Pg1-Ps22 | Cg28-Pg1-Ps23 | Cg28-Pg1-Ps24 |
| Cg28-Pg1-Ps25 | Cg28-Pg1-Ps26 | Cg28-Pg1-Ps27 | Cg28-Pg1-Ps28 |
| Cg28-Pg1-Ps29 | Cg28-Pg1-Ps30 | Cg28-Pg1-Ps31 | Cg28-Pg1-Ps32 |
| Cg28-Pg1-Ps33 | Cg28-Pg1-Ps34 | Cg28-Pg1-Ps35 | Cg28-Pg1-Ps36 |
| Cg28-Pg1-Ps37 | Cg28-Pg1-Ps38 | Cg28-Pg1-Ps39 | Cg28-Pg1-Ps40 |
| Cg28-Pg1-Ps41 | Cg28-Pg1-Ps42 | Cg28-Pg1-Ps43 | Cg28-Pg1-Ps44 |
| Cg28-Pg1-Ps45 | Cg28-Pg1-Ps46 | Cg28-Pg1-Ps47 | Cg28-Pg1-Ps48 |
| Cg28-Pg1-Ps49 | Cg28-Pg1-Ps50 | Cg28-Pg1-Ps51 | Cg28-Pg1-Ps52 |
| Cg28-Pg1-Ps53 | Cg28-Pg1-Ps54 | Cg28-Pg1-Ps55 | Cg28-Pg1-Ps56 |
| Cg28-Pg1-Ps57 | Cg28-Pg1-Ps58 | Cg28-Pg1-Ps59 | Cg28-Pg1-Ps60 |
| Cg28-Pg1-Ps61 | Cg28-Pg1-Ps62 | Cg28-Pg1-Ps63 | Cg28-Pg1-Ps64 |
| Cg28-Pg1-Ps65 | Cg28-Pg1-Ps66 | Cg28-Pg1-Ps67 | Cg28-Pg1-Ps68 |
| Cg28-Pg1-Ps69 | Cg28-Pg1-Ps70 | Cg28-Pg1-Ps71 | Cg28-Pg1-Ps72 |
| Cg28-Pg1-Ps73 | Cg28-Pg1-Ps74 | Cg28-Pg1-Ps75 | Cg28-Pg1-Ps76 |
| Cg28-Pg1-Ps77 | Cg28-Pg1-Ps78 | Cg28-Pg1-Ps79 | Cg28-Pg1-Ps80 |
| Cg28-Pg1-Ps81 | Cg28-Pg1-Ps82 | Cg28-Pg1-Ps83 | Cg28-Pg1-Ps84 |
| Cg28-Pg1-Ps85 | Cg28-Pg1-Ps86 | Cg28-Pg1-Ps87 | Cg28-Pg1-Ps88 |
| Cg28-Pg1-Ps89 | Cg28-Pg1-Ps90 | Cg28-Pg1-Ps91 | Cg28-Pg1-Ps92 |
| Cg28-Pg1-Ps93 | Cg28-Pg1-Ps94 | Cg28-Pg1-Ps95 | Cg28-Pg1-Ps96 |
| Cg28-Pg1-Ps97 | Cg28-Pg1-Ps98 | Cg28-Pg1-Ps99 | Cg28-Pg1-Ps100 |
| Cg28-Pg1-Ps101 | Cg28-Pg1-Ps102 | Cg28-Pg1-Ps103 | Cg28-Pg1-Ps104 |
| Cg28-Pg1-Ps105 | Cg28-Pg1-Ps106 | Cg28-Pg1-Ps107 | Cg28-Pg1-Ps108 |
| Cg28-Pg1-Ps109 | Cg28-Pg1-Ps110 | Cg28-Pg1-Ps111 | Cg28-Pg1-Ps112 |
| Cg28-Pg1-Ps113 | Cg28-Pg1-Ps114 | Cg28-Pg1-Ps115 | Cg28-Pg1-Ps116 |
| Cg28-Pg1-Ps117 | Cg28-Pg1-Ps118 | Cg28-Pg1-Ps119 | Cg28-Pg1-Ps120 |
| Cg28-Pg1-Ps121 | Cg28-Pg1-Ps122 | Cg28-Pg1-Ps123 | Cg28-Pg1-Ps124 |
| Cg28-Pg1-Ps125 | Cg28-Pg1-Ps126 | Cg28-Pg1-Ps127 | Cg28-Pg1-Ps128 |
| Cg28-Pg1-Ps129 | Cg28-Pg1-Ps130 | Cg28-Pg1-Ps131 | Cg28-Pg1-Ps132 |
| Cg28-Pg1-Ps133 | Cg28-Pg1-Ps134 | Cg28-Pg1-Ps135 | Cg28-Pg1-Ps136 |
| Cg28-Pg1-Ps137 | Cg28-Pg1-Ps138 | Cg28-Pg1-Ps139 | Cg28-Pg1-Ps140 |
| Cg28-Pg1-Ps141 | Cg28-Pg1-Ps142 | Cg28-Pg1-Ps143 | Cg28-Pg1-Ps144 |
| Cg28-Pg1-Ps145 | Cg28-Pg1-Ps146 | Cg28-Pg1-Ps147 | Cg28-Pg1-Ps148 |
| Cg28-Pg1-Ps149 | Cg28-Pg1-Ps150 | Cg28-Pg1-Ps151 | Cg28-Pg1-Ps152 |
| Cg28-Pg1-Ps153 | Cg28-Pg1-Ps154 | Cg28-Pg1-Ps155 | Cg28-Pg1-Ps156 |
| Cg28-Pg1-Ps157 | Cg28-Pg1-Ps158 | Cg28-Pg1-Ps159 | Cg28-Pg1-Ps160 |
| Cg28-Pg1-Ps161 | Cg28-Pg1-Ps162 | Cg28-Pg1-Ps163 | Cg28-Pg1-Ps164 |
| Cg28-Pg1-Ps165 | Cg28-Pg1-Ps166 | Cg28-Pg1-Ps167 | Cg28-Pg1-Ps168 |
| Cg28-Pg1-Ps169 | Cg28-Pg1-Ps170 | Cg28-Pg1-Ps171 | Cg28-Pg1-Ps172 |
| Cg28-Pg1-Ps173 | Cg28-Pg1-Ps174 | Cg28-Pg1-Ps175 | Cg28-Pg1-Ps176 |
| Cg28-Pg1-Ps177 | Cg28-Pg1-Ps178 | Cg28-Pg1-Ps179 | Cg28-Pg1-Ps180 |
| Cg28-Pg1-Ps181 | Cg28-Pg1-Ps182 | Cg28-Pg1-Ps183 | Cg28-Pg1-Ps184 |
| Cg28-Pg1-Ps185 | Cg28-Pg1-Ps186 | Cg28-Pg1-Ps187 | Cg28-Pg1-Ps188 |
| Cg28-Pg1-Ps189 | Cg28-Pg1-Ps190 | Cg28-Pg1-Ps191 | Cg28-Pg1-Ps192 |
| Cg28-Pg1-Ps193 | Cg28-Pg1-Ps194 | Cg28-Pg1-Ps195 | Cg28-Pg1-Ps196 |
| Cg28-Pg1-Ps197 | Cg28-Pg1-Ps198 | Cg28-Pg1-Ps199 | Cg28-Pg1-Ps200 |
| Cg28-Pg1-Ps201 | Cg28-Pg1-Ps202 | Cg28-Pg1-Ps203 | Cg28-Pg1-Ps204 |
| Cg28-Pg1-Ps205 | Cg28-Pg1-Ps206 | Cg28-Pg1-Ps207 | Cg28-Pg1-Ps208 |
| Cg28-Pg1-Ps209 | Cg28-Pg1-Ps210 | Cg28-Pg1-Ps211 | Cg28-Pg1-Ps212 |
| Cg28-Pg1-Ps213 | Cg28-Pg1-Ps214 | Cg28-Pg1-Ps215 | Cg28-Pg1-Ps216 |
| Cg28-Pg1-Ps217 | Cg28-Pg1-Ps218 | Cg28-Pg1-Ps219 | Cg28-Pg1-Ps220 |
| Cg28-Pg1-Ps221 | Cg28-Pg1-Ps222 | Cg28-Pg1-Ps223 | Cg28-Pg1-Ps224 |
| Cg28-Pg1-Ps225 | Cg28-Pg1-Ps226 | Cg28-Pg1-Ps227 | Cg28-Pg1-Ps228 |
| Cg28-Pg1-Ps229 | Cg28-Pg1-Ps230 | Cg28-Pg1-Ps231 | Cg28-Pg1-Ps232 |
| Cg28-Pg1-Ps233 | Cg28-Pg1-Ps234 | Cg28-Pg1-Ps235 | Cg28-Pg1-Ps236 |
| Cg28-Pg1-Ps237 | Cg28-Pg1-Ps238 | Cg28-Pg1-Ps239 | Cg28-Pg1-Ps240 |
| Cg28-Pg1-Ps241 | Cg28-Pg1-Ps242 | Cg28-Pg1-Ps243 | |
| Cg29-Pg1-Ps1 | Cg29-Pg1-Ps2 | Cg29-Pg1-Ps3 | Cg29-Pg1-Ps4 |
| Cg29-Pg1-Ps5 | Cg29-Pg1-Ps6 | Cg29-Pg1-Ps7 | Cg29-Pg1-Ps8 |
| Cg29-Pg1-Ps9 | Cg29-Pg1-Ps10 | Cg29-Pg1-Ps11 | Cg29-Pg1-Ps12 |
| Cg29-Pg1-Ps13 | Cg29-Pg1-Ps14 | Cg29-Pg1-Ps15 | Cg29-Pg1-Ps16 |
| Cg29-Pg1-Ps17 | Cg29-Pg1-Ps18 | Cg29-Pg1-Ps19 | Cg29-Pg1-Ps20 |
| Cg29-Pg1-Ps21 | Cg29-Pg1-Ps22 | Cg29-Pg1-Ps23 | Cg29-Pg1-Ps24 |
| Cg29-Pg1-Ps25 | Cg29-Pg1-Ps26 | Cg29-Pg1-Ps27 | Cg29-Pg1-Ps28 |
| Cg29-Pg1-Ps29 | Cg29-Pg1-Ps30 | Cg29-Pg1-Ps31 | Cg29-Pg1-Ps32 |
| Cg29-Pg1-Ps33 | Cg29-Pg1-Ps34 | Cg29-Pg1-Ps35 | Cg29-Pg1-Ps36 |
| Cg29-Pg1-Ps37 | Cg29-Pg1-Ps38 | Cg29-Pg1-Ps39 | Cg29-Pg1-Ps40 |
| Cg29-Pg1-Ps41 | Cg29-Pg1-Ps42 | Cg29-Pg1-Ps43 | Cg29-Pg1-Ps44 |
| Cg29-Pg1-Ps45 | Cg29-Pg1-Ps46 | Cg29-Pg1-Ps47 | Cg29-Pg1-Ps48 |
| Cg29-Pg1-Ps49 | Cg29-Pg1-Ps50 | Cg29-Pg1-Ps51 | Cg29-Pg1-Ps52 |
| Cg29-Pg1-Ps53 | Cg29-Pg1-Ps54 | Cg29-Pg1-Ps55 | Cg29-Pg1-Ps56 |
| Cg29-Pg1-Ps57 | Cg29-Pg1-Ps58 | Cg29-Pg1-Ps59 | Cg29-Pg1-Ps60 |
| Cg29-Pg1-Ps61 | Cg29-Pg1-Ps62 | Cg29-Pg1-Ps63 | Cg29-Pg1-Ps64 |
| Cg29-Pg1-Ps65 | Cg29-Pg1-Ps66 | Cg29-Pg1-Ps67 | Cg29-Pg1-Ps68 |
| Cg29-Pg1-Ps69 | Cg29-Pg1-Ps70 | Cg29-Pg1-Ps71 | Cg29-Pg1-Ps72 |
| Cg29-Pg1-Ps73 | Cg29-Pg1-Ps74 | Cg29-Pg1-Ps75 | Cg29-Pg1-Ps76 |
| Cg29-Pg1-Ps77 | Cg29-Pg1-Ps78 | Cg29-Pg1-Ps79 | Cg29-Pg1-Ps80 |
| Cg29-Pg1-Ps81 | Cg29-Pg1-Ps82 | Cg29-Pg1-Ps83 | Cg29-Pg1-Ps84 |
| Cg29-Pg1-Ps85 | Cg29-Pg1-Ps86 | Cg29-Pg1-Ps87 | Cg29-Pg1-Ps88 |
| Cg29-Pg1-Ps89 | Cg29-Pg1-Ps90 | Cg29-Pg1-Ps91 | Cg29-Pg1-Ps92 |
| Cg29-Pg1-Ps93 | Cg29-Pg1-Ps94 | Cg29-Pg1-Ps95 | Cg29-Pg1-Ps96 |
| Cg29-Pg1-Ps97 | Cg29-Pg1-Ps98 | Cg29-Pg1-Ps99 | Cg29-Pg1-Ps100 |
| Cg29-Pg1-Ps101 | Cg29-Pg1-Ps102 | Cg29-Pg1-Ps103 | Cg29-Pg1-Ps104 |
| Cg29-Pg1-Ps105 | Cg29-Pg1-Ps106 | Cg29-Pg1-Ps107 | Cg29-Pg1-Ps108 |
| Cg29-Pg1-Ps109 | Cg29-Pg1-Ps110 | Cg29-Pg1-Ps111 | Cg29-Pg1-Ps112 |
| Cg29-Pg1-Ps113 | Cg29-Pg1-Ps114 | Cg29-Pg1-Ps115 | Cg29-Pg1-Ps116 |
| Cg29-Pg1-Ps117 | Cg29-Pg1-Ps118 | Cg29-Pg1-Ps119 | Cg29-Pg1-Ps120 |
| Cg29-Pg1-Ps121 | Cg29-Pg1-Ps122 | Cg29-Pg1-Ps123 | Cg29-Pg1-Ps124 |
| Cg29-Pg1-Ps125 | Cg29-Pg1-Ps126 | Cg29-Pg1-Ps127 | Cg29-Pg1-Ps128 |
| Cg29-Pg1-Ps129 | Cg29-Pg1-Ps130 | Cg29-Pg1-Ps131 | Cg29-Pg1-Ps132 |
| Cg29-Pg1-Ps133 | Cg29-Pg1-Ps134 | Cg29-Pg1-Ps135 | Cg29-Pg1-Ps136 |
| Cg29-Pg1-Ps137 | Cg29-Pg1-Ps138 | Cg29-Pg1-Ps139 | Cg29-Pg1-Ps140 |
| Cg29-Pg1-Ps141 | Cg29-Pg1-Ps142 | Cg29-Pg1-Ps143 | Cg29-Pg1-Ps144 |
| Cg29-Pg1-Ps145 | Cg29-Pg1-Ps146 | Cg29-Pg1-Ps147 | Cg29-Pg1-Ps148 |
| Cg29-Pg1-Ps149 | Cg29-Pg1-Ps150 | Cg29-Pg1-Ps151 | Cg29-Pg1-Ps152 |
| Cg29-Pg1-Ps153 | Cg29-Pg1-Ps154 | Cg29-Pg1-Ps155 | Cg29-Pg1-Ps156 |
| Cg29-Pg1-Ps157 | Cg29-Pg1-Ps158 | Cg29-Pg1-Ps159 | Cg29-Pg1-Ps160 |
| Cg29-Pg1-Ps161 | Cg29-Pg1-Ps162 | Cg29-Pg1-Ps163 | Cg29-Pg1-Ps164 |
| Cg29-Pg1-Ps165 | Cg29-Pg1-Ps166 | Cg29-Pg1-Ps167 | Cg29-Pg1-Ps168 |
| Cg29-Pg1-Ps169 | Cg29-Pg1-Ps170 | Cg29-Pg1-Ps171 | Cg29-Pg1-Ps172 |
| Cg29-Pg1-Ps173 | Cg29-Pg1-Ps174 | Cg29-Pg1-Ps175 | Cg29-Pg1-Ps176 |
| Cg29-Pg1-Ps177 | Cg29-Pg1-Ps178 | Cg29-Pg1-Ps179 | Cg29-Pg1-Ps180 |
| Cg29-Pg1-Ps181 | Cg29-Pg1-Ps182 | Cg29-Pg1-Ps183 | Cg29-Pg1-Ps184 |
| Cg29-Pg1-Ps185 | Cg29-Pg1-Ps186 | Cg29-Pg1-Ps187 | Cg29-Pg1-Ps188 |
| Cg29-Pg1-Ps189 | Cg29-Pg1-Ps190 | Cg29-Pg1-Ps191 | Cg29-Pg1-Ps192 |
| Cg29-Pg1-Ps193 | Cg29-Pg1-Ps194 | Cg29-Pg1-Ps195 | Cg29-Pg1-Ps196 |
| Cg29-Pg1-Ps197 | Cg29-Pg1-Ps198 | Cg29-Pg1-Ps199 | Cg29-Pg1-Ps200 |
| Cg29-Pg1-Ps201 | Cg29-Pg1-Ps202 | Cg29-Pg1-Ps203 | Cg29-Pg1-Ps204 |
| Cg29-Pg1-Ps205 | Cg29-Pg1-Ps206 | Cg29-Pg1-Ps207 | Cg29-Pg1-Ps208 |
| Cg29-Pg1-Ps209 | Cg29-Pg1-Ps210 | Cg29-Pg1-Ps211 | Cg29-Pg1-Ps212 |
| Cg29-Pg1-Ps213 | Cg29-Pg1-Ps214 | Cg29-Pg1-Ps215 | Cg29-Pg1-Ps216 |
| Cg29-Pg1-Ps217 | Cg29-Pg1-Ps218 | Cg29-Pg1-Ps219 | Cg29-Pg1-Ps220 |
| Cg29-Pg1-Ps221 | Cg29-Pg1-Ps222 | Cg29-Pg1-Ps223 | Cg29-Pg1-Ps224 |
| Cg29-Pg1-Ps225 | Cg29-Pg1-Ps226 | Cg29-Pg1-Ps227 | Cg29-Pg1-Ps228 |
| Cg29-Pg1-Ps229 | Cg29-Pg1-Ps230 | Cg29-Pg1-Ps231 | Cg29-Pg1-Ps232 |
| Cg29-Pg1-Ps233 | Cg29-Pg1-Ps234 | Cg29-Pg1-Ps235 | Cg29-Pg1-Ps236 |
| Cg29-Pg1-Ps237 | Cg29-Pg1-Ps238 | Cg29-Pg1-Ps239 | Cg29-Pg1-Ps240 |
| Cg29-Pg1-Ps241 | Cg29-Pg1-Ps242 | Cg29-Pg1-Ps243 | |
| Cg30-Pg1-Ps1 | Cg30-Pg1-Ps2 | Cg30-Pg1-Ps3 | Cg30-Pg1-Ps4 |
| Cg30-Pg1-Ps5 | Cg30-Pg1-Ps6 | Cg30-Pg1-Ps7 | Cg30-Pg1-Ps8 |
| Cg30-Pg1-Ps9 | Cg30-Pg1-Ps10 | Cg30-Pg1-Ps11 | Cg30-Pg1-Ps12 |
| Cg30-Pg1-Ps13 | Cg30-Pg1-Ps14 | Cg30-Pg1-Ps15 | Cg30-Pg1-Ps16 |
| Cg30-Pg1-Ps17 | Cg30-Pg1-Ps18 | Cg30-Pg1-Ps19 | Cg30-Pg1-Ps20 |
| Cg30-Pg1-Ps21 | Cg30-Pg1-Ps22 | Cg30-Pg1-Ps23 | Cg30-Pg1-Ps24 |
| Cg30-Pg1-Ps25 | Cg30-Pg1-Ps26 | Cg30-Pg1-Ps27 | Cg30-Pg1-Ps28 |
| Cg30-Pg1-Ps29 | Cg30-Pg1-Ps30 | Cg30-Pg1-Ps31 | Cg30-Pg1-Ps32 |
| Cg30-Pg1-Ps33 | Cg30-Pg1-Ps34 | Cg30-Pg1-Ps35 | Cg30-Pg1-Ps36 |
| Cg30-Pg1-Ps37 | Cg30-Pg1-Ps38 | Cg30-Pg1-Ps39 | Cg30-Pg1-Ps40 |
| Cg30-Pg1-Ps41 | Cg30-Pg1-Ps42 | Cg30-Pg1-Ps43 | Cg30-Pg1-Ps44 |
| Cg30-Pg1-Ps45 | Cg30-Pg1-Ps46 | Cg30-Pg1-Ps47 | Cg30-Pg1-Ps48 |
| Cg30-Pg1-Ps49 | Cg30-Pg1-Ps50 | Cg30-Pg1-Ps51 | Cg30-Pg1-Ps52 |
| Cg30-Pg1-Ps53 | Cg30-Pg1-Ps54 | Cg30-Pg1-Ps55 | Cg30-Pg1-Ps56 |
| Cg30-Pg1-Ps57 | Cg30-Pg1-Ps58 | Cg30-Pg1-Ps59 | Cg30-Pg1-Ps60 |
| Cg30-Pg1-Ps61 | Cg30-Pg1-Ps62 | Cg30-Pg1-Ps63 | Cg30-Pg1-Ps64 |
| Cg30-Pg1-Ps65 | Cg30-Pg1-Ps66 | Cg30-Pg1-Ps67 | Cg30-Pg1-Ps68 |
| Cg30-Pg1-Ps69 | Cg30-Pg1-Ps70 | Cg30-Pg1-Ps71 | Cg30-Pg1-Ps72 |
| Cg30-Pg1-Ps73 | Cg30-Pg1-Ps74 | Cg30-Pg1-Ps75 | Cg30-Pg1-Ps76 |
| Cg30-Pg1-Ps77 | Cg30-Pg1-Ps78 | Cg30-Pg1-Ps79 | Cg30-Pg1-Ps80 |
| Cg30-Pg1-Ps81 | Cg30-Pg1-Ps82 | Cg30-Pg1-Ps83 | Cg30-Pg1-Ps84 |
| Cg30-Pg1-Ps85 | Cg30-Pg1-Ps86 | Cg30-Pg1-Ps87 | Cg30-Pg1-Ps88 |
| Cg30-Pg1-Ps89 | Cg30-Pg1-Ps90 | Cg30-Pg1-Ps91 | Cg30-Pg1-Ps92 |
| Cg30-Pg1-Ps93 | Cg30-Pg1-Ps94 | Cg30-Pg1-Ps95 | Cg30-Pg1-Ps96 |
| Cg30-Pg1-Ps97 | Cg30-Pg1-Ps98 | Cg30-Pg1-Ps99 | Cg30-Pg1-Ps100 |
| Cg30-Pg1-Ps101 | Cg30-Pg1-Ps102 | Cg30-Pg1-Ps103 | Cg30-Pg1-Ps104 |
| Cg30-Pg1-Ps105 | Cg30-Pg1-Ps106 | Cg30-Pg1-Ps107 | Cg30-Pg1-Ps108 |
| Cg30-Pg1-Ps109 | Cg30-Pg1-Ps110 | Cg30-Pg1-Ps111 | Cg30-Pg1-Ps112 |
| Cg30-Pg1-Ps113 | Cg30-Pg1-Ps114 | Cg30-Pg1-Ps115 | Cg30-Pg1-Ps116 |
| Cg30-Pg1-Ps117 | Cg30-Pg1-Ps118 | Cg30-Pg1-Ps119 | Cg30-Pg1-Ps120 |
| Cg30-Pg1-Ps121 | Cg30-Pg1-Ps122 | Cg30-Pg1-Ps123 | Cg30-Pg1-Ps124 |
| Cg30-Pg1-Ps125 | Cg30-Pg1-Ps126 | Cg30-Pg1-Ps127 | Cg30-Pg1-Ps128 |
| Cg30-Pg1-Ps129 | Cg30-Pg1-Ps130 | Cg30-Pg1-Ps131 | Cg30-Pg1-Ps132 |
| Cg30-Pg1-Ps133 | Cg30-Pg1-Ps134 | Cg30-Pg1-Ps135 | Cg30-Pg1-Ps136 |
| Cg30-Pg1-Ps137 | Cg30-Pg1-Ps138 | Cg30-Pg1-Ps139 | Cg30-Pg1-Ps140 |
| Cg30-Pg1-Ps141 | Cg30-Pg1-Ps142 | Cg30-Pg1-Ps143 | Cg30-Pg1-Ps144 |
| Cg30-Pg1-Ps145 | Cg30-Pg1-Ps146 | Cg30-Pg1-Ps147 | Cg30-Pg1-Ps148 |
| Cg30-Pg1-Ps149 | Cg30-Pg1-Ps150 | Cg30-Pg1-Ps151 | Cg30-Pg1-Ps152 |
| Cg30-Pg1-Ps153 | Cg30-Pg1-Ps154 | Cg30-Pg1-Ps155 | Cg30-Pg1-Ps156 |
| Cg30-Pg1-Ps157 | Cg30-Pg1-Ps158 | Cg30-Pg1-Ps159 | Cg30-Pg1-Ps160 |
| Cg30-Pg1-Ps161 | Cg30-Pg1-Ps162 | Cg30-Pg1-Ps163 | Cg30-Pg1-Ps164 |
| Cg30-Pg1-Ps165 | Cg30-Pg1-Ps166 | Cg30-Pg1-Ps167 | Cg30-Pg1-Ps168 |
| Cg30-Pg1-Ps169 | Cg30-Pg1-Ps170 | Cg30-Pg1-Ps171 | Cg30-Pg1-Ps172 |
| Cg30-Pg1-Ps173 | Cg30-Pg1-Ps174 | Cg30-Pg1-Ps175 | Cg30-Pg1-Ps176 |
| Cg30-Pg1-Ps177 | Cg30-Pg1-Ps178 | Cg30-Pg1-Ps179 | Cg30-Pg1-Ps180 |
| Cg30-Pg1-Ps181 | Cg30-Pg1-Ps182 | Cg30-Pg1-Ps183 | Cg30-Pg1-Ps184 |
| Cg30-Pg1-Ps185 | Cg30-Pg1-Ps186 | Cg30-Pg1-Ps187 | Cg30-Pg1-Ps188 |
| Cg30-Pg1-Ps189 | Cg30-Pg1-Ps190 | Cg30-Pg1-Ps191 | Cg30-Pg1-Ps192 |
| Cg30-Pg1-Ps193 | Cg30-Pg1-Ps194 | Cg30-Pg1-Ps195 | Cg30-Pg1-Ps196 |
| Cg30-Pg1-Ps197 | Cg30-Pg1-Ps198 | Cg30-Pg1-Ps199 | Cg30-Pg1-Ps200 |
| Cg30-Pg1-Ps201 | Cg30-Pg1-Ps202 | Cg30-Pg1-Ps203 | Cg30-Pg1-Ps204 |
| Cg30-Pg1-Ps205 | Cg30-Pg1-Ps206 | Cg30-Pg1-Ps207 | Cg30-Pg1-Ps208 |
| Cg30-Pg1-Ps209 | Cg30-Pg1-Ps210 | Cg30-Pg1-Ps211 | Cg30-Pg1-Ps212 |
| Cg30-Pg1-Ps213 | Cg30-Pg1-Ps214 | Cg30-Pg1-Ps215 | Cg30-Pg1-Ps216 |
| Cg30-Pg1-Ps217 | Cg30-Pg1-Ps218 | Cg30-Pg1-Ps219 | Cg30-Pg1-Ps220 |
| Cg30-Pg1-Ps221 | Cg30-Pg1-Ps222 | Cg30-Pg1-Ps223 | Cg30-Pg1-Ps224 |
| Cg30-Pg1-Ps225 | Cg30-Pg1-Ps226 | Cg30-Pg1-Ps227 | Cg30-Pg1-Ps228 |
| Cg30-Pg1-Ps229 | Cg30-Pg1-Ps230 | Cg30-Pg1-Ps231 | Cg30-Pg1-Ps232 |
| Cg30-Pg1-Ps233 | Cg30-Pg1-Ps234 | Cg30-Pg1-Ps235 | Cg30-Pg1-Ps236 |
| Cg30-Pg1-Ps237 | Cg30-Pg1-Ps238 | Cg30-Pg1-Ps239 | Cg30-Pg1-Ps240 |
| Cg30-Pg1-Ps241 | Cg30-Pg1-Ps242 | Cg30-Pg1-Ps243 | |
| Cg31-Pg1-Ps1 | Cg31-Pg1-Ps2 | Cg31-Pg1-Ps3 | Cg31-Pg1-Ps4 |
| Cg31-Pg1-Ps5 | Cg31-Pg1-Ps6 | Cg31-Pg1-Ps7 | Cg31-Pg1-Ps8 |
| Cg31-Pg1-Ps9 | Cg31-Pg1-Ps10 | Cg31-Pg1-Ps11 | Cg31-Pg1-Ps12 |
| Cg31-Pg1-Ps13 | Cg31-Pg1-Ps14 | Cg31-Pg1-Ps15 | Cg31-Pg1-Ps16 |
| Cg31-Pg1-Ps17 | Cg31-Pg1-Ps18 | Cg31-Pg1-Ps19 | Cg31-Pg1-Ps20 |
| Cg31-Pg1-Ps21 | Cg31-Pg1-Ps22 | Cg31-Pg1-Ps23 | Cg31-Pg1-Ps24 |
| Cg31-Pg1-Ps25 | Cg31-Pg1-Ps26 | Cg31-Pg1-Ps27 | Cg31-Pg1-Ps28 |
| Cg31-Pg1-Ps29 | Cg31-Pg1-Ps30 | Cg31-Pg1-Ps31 | Cg31-Pg1-Ps32 |
| Cg31-Pg1-Ps33 | Cg31-Pg1-Ps34 | Cg31-Pg1-Ps35 | Cg31-Pg1-Ps36 |
| Cg31-Pg1-Ps37 | Cg31-Pg1-Ps38 | Cg31-Pg1-Ps39 | Cg31-Pg1-Ps40 |
| Cg31-Pg1-Ps41 | Cg31-Pg1-Ps42 | Cg31-Pg1-Ps43 | Cg31-Pg1-Ps44 |
| Cg31-Pg1-Ps45 | Cg31-Pg1-Ps46 | Cg31-Pg1-Ps47 | Cg31-Pg1-Ps48 |
| Cg31-Pg1-Ps49 | Cg31-Pg1-Ps50 | Cg31-Pg1-Ps51 | Cg31-Pg1-Ps52 |
| Cg31-Pg1-Ps53 | Cg31-Pg1-Ps54 | Cg31-Pg1-Ps55 | Cg31-Pg1-Ps56 |
| Cg31-Pg1-Ps57 | Cg31-Pg1-Ps58 | Cg31-Pg1-Ps59 | Cg31-Pg1-Ps60 |
| Cg31-Pg1-Ps61 | Cg31-Pg1-Ps62 | Cg31-Pg1-Ps63 | Cg31-Pg1-Ps64 |
| Cg31-Pg1-Ps65 | Cg31-Pg1-Ps66 | Cg31-Pg1-Ps67 | Cg31-Pg1-Ps68 |
| Cg31-Pg1-Ps69 | Cg31-Pg1-Ps70 | Cg31-Pg1-Ps71 | Cg31-Pg1-Ps72 |
| Cg31-Pg1-Ps73 | Cg31-Pg1-Ps74 | Cg31-Pg1-Ps75 | Cg31-Pg1-Ps76 |
| Cg31-Pg1-Ps77 | Cg31-Pg1-Ps78 | Cg31-Pg1-Ps79 | Cg31-Pg1-Ps80 |
| Cg31-Pg1-Ps81 | Cg31-Pg1-Ps82 | Cg31-Pg1-Ps83 | Cg31-Pg1-Ps84 |
| Cg31-Pg1-Ps85 | Cg31-Pg1-Ps86 | Cg31-Pg1-Ps87 | Cg31-Pg1-Ps88 |
| Cg31-Pg1-Ps89 | Cg31-Pg1-Ps90 | Cg31-Pg1-Ps91 | Cg31-Pg1-Ps92 |
| Cg31-Pg1-Ps93 | Cg31-Pg1-Ps94 | Cg31-Pg1-Ps95 | Cg31-Pg1-Ps96 |
| Cg31-Pg1-Ps97 | Cg31-Pg1-Ps98 | Cg31-Pg1-Ps99 | Cg31-Pg1-Ps100 |
| Cg31-Pg1-Ps101 | Cg31-Pg1-Ps102 | Cg31-Pg1-Ps103 | Cg31-Pg1-Ps104 |
| Cg31-Pg1-Ps105 | Cg31-Pg1-Ps106 | Cg31-Pg1-Ps107 | Cg31-Pg1-Ps108 |
| Cg31-Pg1-Ps109 | Cg31-Pg1-Ps110 | Cg31-Pg1-Ps111 | Cg31-Pg1-Ps112 |
| Cg31-Pg1-Ps113 | Cg31-Pg1-Ps114 | Cg31-Pg1-Ps115 | Cg31-Pg1-Ps116 |
| Cg31-Pg1-Ps117 | Cg31-Pg1-Ps118 | Cg31-Pg1-Ps119 | Cg31-Pg1-Ps120 |
| Cg31-Pg1-Ps121 | Cg31-Pg1-Ps122 | Cg31-Pg1-Ps123 | Cg31-Pg1-Ps124 |
| Cg31-Pg1-Ps125 | Cg31-Pg1-Ps126 | Cg31-Pg1-Ps127 | Cg31-Pg1-Ps128 |
| Cg31-Pg1-Ps129 | Cg31-Pg1-Ps130 | Cg31-Pg1-Ps131 | Cg31-Pg1-Ps132 |
| Cg31-Pg1-Ps133 | Cg31-Pg1-Ps134 | Cg31-Pg1-Ps135 | Cg31-Pg1-Ps136 |
| Cg31-Pg1-Ps137 | Cg31-Pg1-Ps138 | Cg31-Pg1-Ps139 | Cg31-Pg1-Ps140 |
| Cg31-Pg1-Ps141 | Cg31-Pg1-Ps142 | Cg31-Pg1-Ps143 | Cg31-Pg1-Ps144 |
| Cg31-Pg1-Ps145 | Cg31-Pg1-Ps146 | Cg31-Pg1-Ps147 | Cg31-Pg1-Ps148 |
| Cg31-Pg1-Ps149 | Cg31-Pg1-Ps150 | Cg31-Pg1-Ps151 | Cg31-Pg1-Ps152 |
| Cg31-Pg1-Ps153 | Cg31-Pg1-Ps154 | Cg31-Pg1-Ps155 | Cg31-Pg1-Ps156 |
| Cg31-Pg1-Ps157 | Cg31-Pg1-Ps158 | Cg31-Pg1-Ps159 | Cg31-Pg1-Ps160 |
| Cg31-Pg1-Ps161 | Cg31-Pg1-Ps162 | Cg31-Pg1-Ps163 | Cg31-Pg1-Ps164 |
| Cg31-Pg1-Ps165 | Cg31-Pg1-Ps166 | Cg31-Pg1-Ps167 | Cg31-Pg1-Ps168 |
| Cg31-Pg1-Ps169 | Cg31-Pg1-Ps170 | Cg31-Pg1-Ps171 | Cg31-Pg1-Ps172 |
| Cg31-Pg1-Ps173 | Cg31-Pg1-Ps174 | Cg31-Pg1-Ps175 | Cg31-Pg1-Ps176 |
| Cg31-Pg1-Ps177 | Cg31-Pg1-Ps178 | Cg31-Pg1-Ps179 | Cg31-Pg1-Ps180 |
| Cg31-Pg1-Ps181 | Cg31-Pg1-Ps182 | Cg31-Pg1-Ps183 | Cg31-Pg1-Ps184 |
| Cg31-Pg1-Ps185 | Cg31-Pg1-Ps186 | Cg31-Pg1-Ps187 | Cg31-Pg1-Ps188 |
| Cg31-Pg1-Ps189 | Cg31-Pg1-Ps190 | Cg31-Pg1-Ps191 | Cg31-Pg1-Ps192 |
| Cg31-Pg1-Ps193 | Cg31-Pg1-Ps194 | Cg31-Pg1-Ps195 | Cg31-Pg1-Ps196 |
| Cg31-Pg1-Ps197 | Cg31-Pg1-Ps198 | Cg31-Pg1-Ps199 | Cg31-Pg1-Ps200 |
| Cg31-Pg1-Ps201 | Cg31-Pg1-Ps202 | Cg31-Pg1-Ps203 | Cg31-Pg1-Ps204 |
| Cg31-Pg1-Ps205 | Cg31-Pg1-Ps206 | Cg31-Pg1-Ps207 | Cg31-Pg1-Ps208 |
| Cg31-Pg1-Ps209 | Cg31-Pg1-Ps210 | Cg31-Pg1-Ps211 | Cg31-Pg1-Ps212 |
| Cg31-Pg1-Ps213 | Cg31-Pg1-Ps214 | Cg31-Pg1-Ps215 | Cg31-Pg1-Ps216 |
| Cg31-Pg1-Ps217 | Cg31-Pg1-Ps218 | Cg31-Pg1-Ps219 | Cg31-Pg1-Ps220 |
| Cg31-Pg1-Ps221 | Cg31-Pg1-Ps222 | Cg31-Pg1-Ps223 | Cg31-Pg1-Ps224 |
| Cg31-Pg1-Ps225 | Cg31-Pg1-Ps226 | Cg31-Pg1-Ps227 | Cg31-Pg1-Ps228 |
| Cg31-Pg1-Ps229 | Cg31-Pg1-Ps230 | Cg31-Pg1-Ps231 | Cg31-Pg1-Ps232 |
| Cg31-Pg1-Ps233 | Cg31-Pg1-Ps234 | Cg31-Pg1-Ps235 | Cg31-Pg1-Ps236 |
| Cg31-Pg1-Ps237 | Cg31-Pg1-Ps238 | Cg31-Pg1-Ps239 | Cg31-Pg1-Ps240 |
| Cg31-Pg1-Ps241 | Cg31-Pg1-Ps242 | Cg31-Pg1-Ps243 | |
| Cg32-Pg1-Ps1 | Cg32-Pg1-Ps2 | Cg32-Pg1-Ps3 | Cg32-Pg1-Ps4 |
| Cg32-Pg1-Ps5 | Cg32-Pg1-Ps6 | Cg32-Pg1-Ps7 | Cg32-Pg1-Ps8 |
| Cg32-Pg1-Ps9 | Cg32-Pg1-Ps10 | Cg32-Pg1-Ps11 | Cg32-Pg1-Ps12 |
| Cg32-Pg1-Ps13 | Cg32-Pg1-Ps14 | Cg32-Pg1-Ps15 | Cg32-Pg1-Ps16 |
| Cg32-Pg1-Ps17 | Cg32-Pg1-Ps18 | Cg32-Pg1-Ps19 | Cg32-Pg1-Ps20 |
| Cg32-Pg1-Ps21 | Cg32-Pg1-Ps22 | Cg32-Pg1-Ps23 | Cg32-Pg1-Ps24 |
| Cg32-Pg1-Ps25 | Cg32-Pg1-Ps26 | Cg32-Pg1-Ps27 | Cg32-Pg1-Ps28 |
| Cg32-Pg1-Ps29 | Cg32-Pg1-Ps30 | Cg32-Pg1-Ps31 | Cg32-Pg1-Ps32 |
| Cg32-Pg1-Ps33 | Cg32-Pg1-Ps34 | Cg32-Pg1-Ps35 | Cg32-Pg1-Ps36 |
| Cg32-Pg1-Ps37 | Cg32-Pg1-Ps38 | Cg32-Pg1-Ps39 | Cg32-Pg1-Ps40 |
| Cg32-Pg1-Ps41 | Cg32-Pg1-Ps42 | Cg32-Pg1-Ps43 | Cg32-Pg1-Ps44 |
| Cg32-Pg1-Ps45 | Cg32-Pg1-Ps46 | Cg32-Pg1-Ps47 | Cg32-Pg1-Ps48 |
| Cg32-Pg1-Ps49 | Cg32-Pg1-Ps50 | Cg32-Pg1-Ps51 | Cg32-Pg1-Ps52 |
| Cg32-Pg1-Ps53 | Cg32-Pg1-Ps54 | Cg32-Pg1-Ps55 | Cg32-Pg1-Ps56 |
| Cg32-Pg1-Ps57 | Cg32-Pg1-Ps58 | Cg32-Pg1-Ps59 | Cg32-Pg1-Ps60 |
| Cg32-Pg1-Ps61 | Cg32-Pg1-Ps62 | Cg32-Pg1-Ps63 | Cg32-Pg1-Ps64 |
| Cg32-Pg1-Ps65 | Cg32-Pg1-Ps66 | Cg32-Pg1-Ps67 | Cg32-Pg1-Ps68 |
| Cg32-Pg1-Ps69 | Cg32-Pg1-Ps70 | Cg32-Pg1-Ps71 | Cg32-Pg1-Ps72 |
| Cg32-Pg1-Ps73 | Cg32-Pg1-Ps74 | Cg32-Pg1-Ps75 | Cg32-Pg1-Ps76 |
| Cg32-Pg1-Ps77 | Cg32-Pg1-Ps78 | Cg32-Pg1-Ps79 | Cg32-Pg1-Ps80 |
| Cg32-Pg1-Ps81 | Cg32-Pg1-Ps82 | Cg32-Pg1-Ps83 | Cg32-Pg1-Ps84 |
| Cg32-Pg1-Ps85 | Cg32-Pg1-Ps86 | Cg32-Pg1-Ps87 | Cg32-Pg1-Ps88 |
| Cg32-Pg1-Ps89 | Cg32-Pg1-Ps90 | Cg32-Pg1-Ps91 | Cg32-Pg1-Ps92 |
| Cg32-Pg1-Ps93 | Cg32-Pg1-Ps94 | Cg32-Pg1-Ps95 | Cg32-Pg1-Ps96 |
| Cg32-Pg1-Ps97 | Cg32-Pg1-Ps98 | Cg32-Pg1-Ps99 | Cg32-Pg1-Ps100 |
| Cg32-Pg1-Ps101 | Cg32-Pg1-Ps102 | Cg32-Pg1-Ps103 | Cg32-Pg1-Ps104 |
| Cg32-Pg1-Ps105 | Cg32-Pg1-Ps106 | Cg32-Pg1-Ps107 | Cg32-Pg1-Ps108 |
| Cg32-Pg1-Ps109 | Cg32-Pg1-Ps110 | Cg32-Pg1-Ps111 | Cg32-Pg1-Ps112 |
| Cg32-Pg1-Ps113 | Cg32-Pg1-Ps114 | Cg32-Pg1-Ps115 | Cg32-Pg1-Ps116 |
| Cg32-Pg1-Ps117 | Cg32-Pg1-Ps118 | Cg32-Pg1-Ps119 | Cg32-Pg1-Ps120 |
| Cg32-Pg1-Ps121 | Cg32-Pg1-Ps122 | Cg32-Pg1-Ps123 | Cg32-Pg1-Ps124 |
| Cg32-Pg1-Ps125 | Cg32-Pg1-Ps126 | Cg32-Pg1-Ps127 | Cg32-Pg1-Ps128 |
| Cg32-Pg1-Ps129 | Cg32-Pg1-Ps130 | Cg32-Pg1-Ps131 | Cg32-Pg1-Ps132 |
| Cg32-Pg1-Ps133 | Cg32-Pg1-Ps134 | Cg32-Pg1-Ps135 | Cg32-Pg1-Ps136 |
| Cg32-Pg1-Ps137 | Cg32-Pg1-Ps138 | Cg32-Pg1-Ps139 | Cg32-Pg1-Ps140 |
| Cg32-Pg1-Ps141 | Cg32-Pg1-Ps142 | Cg32-Pg1-Ps143 | Cg32-Pg1-Ps144 |
| Cg32-Pg1-Ps145 | Cg32-Pg1-Ps146 | Cg32-Pg1-Ps147 | Cg32-Pg1-Ps148 |
| Cg32-Pg1-Ps149 | Cg32-Pg1-Ps150 | Cg32-Pg1-Ps151 | Cg32-Pg1-Ps152 |
| Cg32-Pg1-Ps153 | Cg32-Pg1-Ps154 | Cg32-Pg1-Ps155 | Cg32-Pg1-Ps156 |
| Cg32-Pg1-Ps157 | Cg32-Pg1-Ps158 | Cg32-Pg1-Ps159 | Cg32-Pg1-Ps160 |
| Cg32-Pg1-Ps161 | Cg32-Pg1-Ps162 | Cg32-Pg1-Ps163 | Cg32-Pg1-Ps164 |
| Cg32-Pg1-Ps165 | Cg32-Pg1-Ps166 | Cg32-Pg1-Ps167 | Cg32-Pg1-Ps168 |
| Cg32-Pg1-Ps169 | Cg32-Pg1-Ps170 | Cg32-Pg1-Ps171 | Cg32-Pg1-Ps172 |
| Cg32-Pg1-Ps173 | Cg32-Pg1-Ps174 | Cg32-Pg1-Ps175 | Cg32-Pg1-Ps176 |
| Cg32-Pg1-Ps177 | Cg32-Pg1-Ps178 | Cg32-Pg1-Ps179 | Cg32-Pg1-Ps180 |
| Cg32-Pg1-Ps181 | Cg32-Pg1-Ps182 | Cg32-Pg1-Ps183 | Cg32-Pg1-Ps184 |
| Cg32-Pg1-Ps185 | Cg32-Pg1-Ps186 | Cg32-Pg1-Ps187 | Cg32-Pg1-Ps188 |
| Cg32-Pg1-Ps189 | Cg32-Pg1-Ps190 | Cg32-Pg1-Ps191 | Cg32-Pg1-Ps192 |
| Cg32-Pg1-Ps193 | Cg32-Pg1-Ps194 | Cg32-Pg1-Ps195 | Cg32-Pg1-Ps196 |
| Cg32-Pg1-Ps197 | Cg32-Pg1-Ps198 | Cg32-Pg1-Ps199 | Cg32-Pg1-Ps200 |
| Cg32-Pg1-Ps201 | Cg32-Pg1-Ps202 | Cg32-Pg1-Ps203 | Cg32-Pg1-Ps204 |
| Cg32-Pg1-Ps205 | Cg32-Pg1-Ps206 | Cg32-Pg1-Ps207 | Cg32-Pg1-Ps208 |
| Cg32-Pg1-Ps209 | Cg32-Pg1-Ps210 | Cg32-Pg1-Ps211 | Cg32-Pg1-Ps212 |
| Cg32-Pg1-Ps213 | Cg32-Pg1-Ps214 | Cg32-Pg1-Ps215 | Cg32-Pg1-Ps216 |
| Cg32-Pg1-Ps217 | Cg32-Pg1-Ps218 | Cg32-Pg1-Ps219 | Cg32-Pg1-Ps220 |
| Cg32-Pg1-Ps221 | Cg32-Pg1-Ps222 | Cg32-Pg1-Ps223 | Cg32-Pg1-Ps224 |
| Cg32-Pg1-Ps225 | Cg32-Pg1-Ps226 | Cg32-Pg1-Ps227 | Cg32-Pg1-Ps228 |
| Cg32-Pg1-Ps229 | Cg32-Pg1-Ps230 | Cg32-Pg1-Ps231 | Cg32-Pg1-Ps232 |
| Cg32-Pg1-Ps233 | Cg32-Pg1-Ps234 | Cg32-Pg1-Ps235 | Cg32-Pg1-Ps236 |
| Cg32-Pg1-Ps237 | Cg32-Pg1-Ps238 | Cg32-Pg1-Ps239 | Cg32-Pg1-Ps240 |
| Cg32-Pg1-Ps241 | Cg32-Pg1-Ps242 | Cg32-Pg1-Ps243 | |
| Cg33-Pg1-Ps1 | Cg33-Pg1-Ps2 | Cg33-Pg1-Ps3 | Cg33-Pg1-Ps4 |
| Cg33-Pg1-Ps5 | Cg33-Pg1-Ps6 | Cg33-Pg1-Ps7 | Cg33-Pg1-Ps8 |
| Cg33-Pg1-Ps9 | Cg33-Pg1-Ps10 | Cg33-Pg1-Ps11 | Cg33-Pg1-Ps12 |
| Cg33-Pg1-Ps13 | Cg33-Pg1-Ps14 | Cg33-Pg1-Ps15 | Cg33-Pg1-Ps16 |
| Cg33-Pg1-Ps17 | Cg33-Pg1-Ps18 | Cg33-Pg1-Ps19 | Cg33-Pg1-Ps20 |
| Cg33-Pg1-Ps21 | Cg33-Pg1-Ps22 | Cg33-Pg1-Ps23 | Cg33-Pg1-Ps24 |
| Cg33-Pg1-Ps25 | Cg33-Pg1-Ps26 | Cg33-Pg1-Ps27 | Cg33-Pg1-Ps28 |
| Cg33-Pg1-Ps29 | Cg33-Pg1-Ps30 | Cg33-Pg1-Ps31 | Cg33-Pg1-Ps32 |
| Cg33-Pg1-Ps33 | Cg33-Pg1-Ps34 | Cg33-Pg1-Ps35 | Cg33-Pg1-Ps36 |
| Cg33-Pg1-Ps37 | Cg33-Pg1-Ps38 | Cg33-Pg1-Ps39 | Cg33-Pg1-Ps40 |
| Cg33-Pg1-Ps41 | Cg33-Pg1-Ps42 | Cg33-Pg1-Ps43 | Cg33-Pg1-Ps44 |
| Cg33-Pg1-Ps45 | Cg33-Pg1-Ps46 | Cg33-Pg1-Ps47 | Cg33-Pg1-Ps48 |
| Cg33-Pg1-Ps49 | Cg33-Pg1-Ps50 | Cg33-Pg1-Ps51 | Cg33-Pg1-Ps52 |
| Cg33-Pg1-Ps53 | Cg33-Pg1-Ps54 | Cg33-Pg1-Ps55 | Cg33-Pg1-Ps56 |
| Cg33-Pg1-Ps57 | Cg33-Pg1-Ps58 | Cg33-Pg1-Ps59 | Cg33-Pg1-Ps60 |
| Cg33-Pg1-Ps61 | Cg33-Pg1-Ps62 | Cg33-Pg1-Ps63 | Cg33-Pg1-Ps64 |
| Cg33-Pg1-Ps65 | Cg33-Pg1-Ps66 | Cg33-Pg1-Ps67 | Cg33-Pg1-Ps68 |
| Cg33-Pg1-Ps69 | Cg33-Pg1-Ps70 | Cg33-Pg1-Ps71 | Cg33-Pg1-Ps72 |
| Cg33-Pg1-Ps73 | Cg33-Pg1-Ps74 | Cg33-Pg1-Ps75 | Cg33-Pg1-Ps76 |
| Cg33-Pg1-Ps77 | Cg33-Pg1-Ps78 | Cg33-Pg1-Ps79 | Cg33-Pg1-Ps80 |
| Cg33-Pg1-Ps81 | Cg33-Pg1-Ps82 | Cg33-Pg1-Ps83 | Cg33-Pg1-Ps84 |
| Cg33-Pg1-Ps85 | Cg33-Pg1-Ps86 | Cg33-Pg1-Ps87 | Cg33-Pg1-Ps88 |
| Cg33-Pg1-Ps89 | Cg33-Pg1-Ps90 | Cg33-Pg1-Ps91 | Cg33-Pg1-Ps92 |
| Cg33-Pg1-Ps93 | Cg33-Pg1-Ps94 | Cg33-Pg1-Ps95 | Cg33-Pg1-Ps96 |
| Cg33-Pg1-Ps97 | Cg33-Pg1-Ps98 | Cg33-Pg1-Ps99 | Cg33-Pg1-Ps100 |
| Cg33-Pg1-Ps101 | Cg33-Pg1-Ps102 | Cg33-Pg1-Ps103 | Cg33-Pg1-Ps104 |
| Cg33-Pg1-Ps105 | Cg33-Pg1-Ps106 | Cg33-Pg1-Ps107 | Cg33-Pg1-Ps108 |
| Cg33-Pg1-Ps109 | Cg33-Pg1-Ps110 | Cg33-Pg1-Ps111 | Cg33-Pg1-Ps112 |
| Cg33-Pg1-Ps113 | Cg33-Pg1-Ps114 | Cg33-Pg1-Ps115 | Cg33-Pg1-Ps116 |
| Cg33-Pg1-Ps117 | Cg33-Pg1-Ps118 | Cg33-Pg1-Ps119 | Cg33-Pg1-Ps120 |
| Cg33-Pg1-Ps121 | Cg33-Pg1-Ps122 | Cg33-Pg1-Ps123 | Cg33-Pg1-Ps124 |
| Cg33-Pg1-Ps125 | Cg33-Pg1-Ps126 | Cg33-Pg1-Ps127 | Cg33-Pg1-Ps128 |
| Cg33-Pg1-Ps129 | Cg33-Pg1-Ps130 | Cg33-Pg1-Ps131 | Cg33-Pg1-Ps132 |
| Cg33-Pg1-Ps133 | Cg33-Pg1-Ps134 | Cg33-Pg1-Ps135 | Cg33-Pg1-Ps136 |
| Cg33-Pg1-Ps137 | Cg33-Pg1-Ps138 | Cg33-Pg1-Ps139 | Cg33-Pg1-Ps140 |
| Cg33-Pg1-Ps141 | Cg33-Pg1-Ps142 | Cg33-Pg1-Ps143 | Cg33-Pg1-Ps144 |
| Cg33-Pg1-Ps145 | Cg33-Pg1-Ps146 | Cg33-Pg1-Ps147 | Cg33-Pg1-Ps148 |
| Cg33-Pg1-Ps149 | Cg33-Pg1-Ps150 | Cg33-Pg1-Ps151 | Cg33-Pg1-Ps152 |
| Cg33-Pg1-Ps153 | Cg33-Pg1-Ps154 | Cg33-Pg1-Ps155 | Cg33-Pg1-Ps156 |
| Cg33-Pg1-Ps157 | Cg33-Pg1-Ps158 | Cg33-Pg1-Ps159 | Cg33-Pg1-Ps160 |
| Cg33-Pg1-Ps161 | Cg33-Pg1-Ps162 | Cg33-Pg1-Ps163 | Cg33-Pg1-Ps164 |
| Cg33-Pg1-Ps165 | Cg33-Pg1-Ps166 | Cg33-Pg1-Ps167 | Cg33-Pg1-Ps168 |
| Cg33-Pg1-Ps169 | Cg33-Pg1-Ps170 | Cg33-Pg1-Ps171 | Cg33-Pg1-Ps172 |
| Cg33-Pg1-Ps173 | Cg33-Pg1-Ps174 | Cg33-Pg1-Ps175 | Cg33-Pg1-Ps176 |
| Cg33-Pg1-Ps177 | Cg33-Pg1-Ps178 | Cg33-Pg1-Ps179 | Cg33-Pg1-Ps180 |
| Cg33-Pg1-Ps181 | Cg33-Pg1-Ps182 | Cg33-Pg1-Ps183 | Cg33-Pg1-Ps184 |
| Cg33-Pg1-Ps185 | Cg33-Pg1-Ps186 | Cg33-Pg1-Ps187 | Cg33-Pg1-Ps188 |
| Cg33-Pg1-Ps189 | Cg33-Pg1-Ps190 | Cg33-Pg1-Ps191 | Cg33-Pg1-Ps192 |
| Cg33-Pg1-Ps193 | Cg33-Pg1-Ps194 | Cg33-Pg1-Ps195 | Cg33-Pg1-Ps196 |
| Cg33-Pg1-Ps197 | Cg33-Pg1-Ps198 | Cg33-Pg1-Ps199 | Cg33-Pg1-Ps200 |
| Cg33-Pg1-Ps201 | Cg33-Pg1-Ps202 | Cg33-Pg1-Ps203 | Cg33-Pg1-Ps204 |
| Cg33-Pg1-Ps205 | Cg33-Pg1-Ps206 | Cg33-Pg1-Ps207 | Cg33-Pg1-Ps208 |
| Cg33-Pg1-Ps209 | Cg33-Pg1-Ps210 | Cg33-Pg1-Ps211 | Cg33-Pg1-Ps212 |
| Cg33-Pg1-Ps213 | Cg33-Pg1-Ps214 | Cg33-Pg1-Ps215 | Cg33-Pg1-Ps216 |
| Cg33-Pg1-Ps217 | Cg33-Pg1-Ps218 | Cg33-Pg1-Ps219 | Cg33-Pg1-Ps220 |
| Cg33-Pg1-Ps221 | Cg33-Pg1-Ps222 | Cg33-Pg1-Ps223 | Cg33-Pg1-Ps224 |
| Cg33-Pg1-Ps225 | Cg33-Pg1-Ps226 | Cg33-Pg1-Ps227 | Cg33-Pg1-Ps228 |
| Cg33-Pg1-Ps229 | Cg33-Pg1-Ps230 | Cg33-Pg1-Ps231 | Cg33-Pg1-Ps232 |
| Cg33-Pg1-Ps233 | Cg33-Pg1-Ps234 | Cg33-Pg1-Ps235 | Cg33-Pg1-Ps236 |
| Cg33-Pg1-Ps237 | Cg33-Pg1-Ps238 | Cg33-Pg1-Ps239 | Cg33-Pg1-Ps240 |
| Cg33-Pg1-Ps241 | Cg33-Pg1-Ps242 | Cg33-Pg1-Ps243 | |
| Cg34-Pg1-Ps1 | Cg34-Pg1-Ps2 | Cg34-Pg1-Ps3 | Cg34-Pg1-Ps4 |
| Cg34-Pg1-Ps5 | Cg34-Pg1-Ps6 | Cg34-Pg1-Ps7 | Cg34-Pg1-Ps8 |
| Cg34-Pg1-Ps9 | Cg34-Pg1-Ps10 | Cg34-Pg1-Ps11 | Cg34-Pg1-Ps12 |
| Cg34-Pg1-Ps13 | Cg34-Pg1-Ps14 | Cg34-Pg1-Ps15 | Cg34-Pg1-Ps16 |
| Cg34-Pg1-Ps17 | Cg34-Pg1-Ps18 | Cg34-Pg1-Ps19 | Cg34-Pg1-Ps20 |
| Cg34-Pg1-Ps21 | Cg34-Pg1-Ps22 | Cg34-Pg1-Ps23 | Cg34-Pg1-Ps24 |
| Cg34-Pg1-Ps25 | Cg34-Pg1-Ps26 | Cg34-Pg1-Ps27 | Cg34-Pg1-Ps28 |
| Cg34-Pg1-Ps29 | Cg34-Pg1-Ps30 | Cg34-Pg1-Ps31 | Cg34-Pg1-Ps32 |
| Cg34-Pg1-Ps33 | Cg34-Pg1-Ps34 | Cg34-Pg1-Ps35 | Cg34-Pg1-Ps36 |
| Cg34-Pg1-Ps37 | Cg34-Pg1-Ps38 | Cg34-Pg1-Ps39 | Cg34-Pg1-Ps40 |
| Cg34-Pg1-Ps41 | Cg34-Pg1-Ps42 | Cg34-Pg1-Ps43 | Cg34-Pg1-Ps44 |
| Cg34-Pg1-Ps45 | Cg34-Pg1-Ps46 | Cg34-Pg1-Ps47 | Cg34-Pg1-Ps48 |
| Cg34-Pg1-Ps49 | Cg34-Pg1-Ps50 | Cg34-Pg1-Ps51 | Cg34-Pg1-Ps52 |
| Cg34-Pg1-Ps53 | Cg34-Pg1-Ps54 | Cg34-Pg1-Ps55 | Cg34-Pg1-Ps56 |
| Cg34-Pg1-Ps57 | Cg34-Pg1-Ps58 | Cg34-Pg1-Ps59 | Cg34-Pg1-Ps60 |
| Cg34-Pg1-Ps61 | Cg34-Pg1-Ps62 | Cg34-Pg1-Ps63 | Cg34-Pg1-Ps64 |
| Cg34-Pg1-Ps65 | Cg34-Pg1-Ps66 | Cg34-Pg1-Ps67 | Cg34-Pg1-Ps68 |
| Cg34-Pg1-Ps69 | Cg34-Pg1-Ps70 | Cg34-Pg1-Ps71 | Cg34-Pg1-Ps72 |
| Cg34-Pg1-Ps73 | Cg34-Pg1-Ps74 | Cg34-Pg1-Ps75 | Cg34-Pg1-Ps76 |
| Cg34-Pg1-Ps77 | Cg34-Pg1-Ps78 | Cg34-Pg1-Ps79 | Cg34-Pg1-Ps80 |
| Cg34-Pg1-Ps81 | Cg34-Pg1-Ps82 | Cg34-Pg1-Ps83 | Cg34-Pg1-Ps84 |
| Cg34-Pg1-Ps85 | Cg34-Pg1-Ps86 | Cg34-Pg1-Ps87 | Cg34-Pg1-Ps88 |
| Cg34-Pg1-Ps89 | Cg34-Pg1-Ps90 | Cg34-Pg1-Ps91 | Cg34-Pg1-Ps92 |
| Cg34-Pg1-Ps93 | Cg34-Pg1-Ps94 | Cg34-Pg1-Ps95 | Cg34-Pg1-Ps96 |
| Cg34-Pg1-Ps97 | Cg34-Pg1-Ps98 | Cg34-Pg1-Ps99 | Cg34-Pg1-Ps100 |
| Cg34-Pg1-Ps101 | Cg34-Pg1-Ps102 | Cg34-Pg1-Ps103 | Cg34-Pg1-Ps104 |
| Cg34-Pg1-Ps105 | Cg34-Pg1-Ps106 | Cg34-Pg1-Ps107 | Cg34-Pg1-Ps108 |
| Cg34-Pg1-Ps109 | Cg34-Pg1-Ps110 | Cg34-Pg1-Ps111 | Cg34-Pg1-Ps112 |
| Cg34-Pg1-Ps113 | Cg34-Pg1-Ps114 | Cg34-Pg1-Ps115 | Cg34-Pg1-Ps116 |
| Cg34-Pg1-Ps117 | Cg34-Pg1-Ps118 | Cg34-Pg1-Ps119 | Cg34-Pg1-Ps120 |
| Cg34-Pg1-Ps121 | Cg34-Pg1-Ps122 | Cg34-Pg1-Ps123 | Cg34-Pg1-Ps124 |
| Cg34-Pg1-Ps125 | Cg34-Pg1-Ps126 | Cg34-Pg1-Ps127 | Cg34-Pg1-Ps128 |
| Cg34-Pg1-Ps129 | Cg34-Pg1-Ps130 | Cg34-Pg1-Ps131 | Cg34-Pg1-Ps132 |
| Cg34-Pg1-Ps133 | Cg34-Pg1-Ps134 | Cg34-Pg1-Ps135 | Cg34-Pg1-Ps136 |
| Cg34-Pg1-Ps137 | Cg34-Pg1-Ps138 | Cg34-Pg1-Ps139 | Cg34-Pg1-Ps140 |
| Cg34-Pg1-Ps141 | Cg34-Pg1-Ps142 | Cg34-Pg1-Ps143 | Cg34-Pg1-Ps144 |
| Cg34-Pg1-Ps145 | Cg34-Pg1-Ps146 | Cg34-Pg1-Ps147 | Cg34-Pg1-Ps148 |
| Cg34-Pg1-Ps149 | Cg34-Pg1-Ps150 | Cg34-Pg1-Ps151 | Cg34-Pg1-Ps152 |
| Cg34-Pg1-Ps153 | Cg34-Pg1-Ps154 | Cg34-Pg1-Ps155 | Cg34-Pg1-Ps156 |
| Cg34-Pg1-Ps157 | Cg34-Pg1-Ps158 | Cg34-Pg1-Ps159 | Cg34-Pg1-Ps160 |
| Cg34-Pg1-Ps161 | Cg34-Pg1-Ps162 | Cg34-Pg1-Ps163 | Cg34-Pg1-Ps164 |
| Cg34-Pg1-Ps165 | Cg34-Pg1-Ps166 | Cg34-Pg1-Ps167 | Cg34-Pg1-Ps168 |
| Cg34-Pg1-Ps169 | Cg34-Pg1-Ps170 | Cg34-Pg1-Ps171 | Cg34-Pg1-Ps172 |
| Cg34-Pg1-Ps173 | Cg34-Pg1-Ps174 | Cg34-Pg1-Ps175 | Cg34-Pg1-Ps176 |
| Cg34-Pg1-Ps177 | Cg34-Pg1-Ps178 | Cg34-Pg1-Ps179 | Cg34-Pg1-Ps180 |
| Cg34-Pg1-Ps181 | Cg34-Pg1-Ps182 | Cg34-Pg1-Ps183 | Cg34-Pg1-Ps184 |
| Cg34-Pg1-Ps185 | Cg34-Pg1-Ps186 | Cg34-Pg1-Ps187 | Cg34-Pg1-Ps188 |
| Cg34-Pg1-Ps189 | Cg34-Pg1-Ps190 | Cg34-Pg1-Ps191 | Cg34-Pg1-Ps192 |
| Cg34-Pg1-Ps193 | Cg34-Pg1-Ps194 | Cg34-Pg1-Ps195 | Cg34-Pg1-Ps196 |
| Cg34-Pg1-Ps197 | Cg34-Pg1-Ps198 | Cg34-Pg1-Ps199 | Cg34-Pg1-Ps200 |
| Cg34-Pg1-Ps201 | Cg34-Pg1-Ps202 | Cg34-Pg1-Ps203 | Cg34-Pg1-Ps204 |
| Cg34-Pg1-Ps205 | Cg34-Pg1-Ps206 | Cg34-Pg1-Ps207 | Cg34-Pg1-Ps208 |
| Cg34-Pg1-Ps209 | Cg34-Pg1-Ps210 | Cg34-Pg1-Ps211 | Cg34-Pg1-Ps212 |
| Cg34-Pg1-Ps213 | Cg34-Pg1-Ps214 | Cg34-Pg1-Ps215 | Cg34-Pg1-Ps216 |
| Cg34-Pg1-Ps217 | Cg34-Pg1-Ps218 | Cg34-Pg1-Ps219 | Cg34-Pg1-Ps220 |
| Cg34-Pg1-Ps221 | Cg34-Pg1-Ps222 | Cg34-Pg1-Ps223 | Cg34-Pg1-Ps224 |
| Cg34-Pg1-Ps225 | Cg34-Pg1-Ps226 | Cg34-Pg1-Ps227 | Cg34-Pg1-Ps228 |
| Cg34-Pg1-Ps229 | Cg34-Pg1-Ps230 | Cg34-Pg1-Ps231 | Cg34-Pg1-Ps232 |
| Cg34-Pg1-Ps233 | Cg34-Pg1-Ps234 | Cg34-Pg1-Ps235 | Cg34-Pg1-Ps236 |
| Cg34-Pg1-Ps237 | Cg34-Pg1-Ps238 | Cg34-Pg1-Ps239 | Cg34-Pg1-Ps240 |
| Cg34-Pg1-Ps241 | Cg34-Pg1-Ps242 | Cg34-Pg1-Ps243 | |
| Cg38-Pg1-Ps1 | Cg38-Pg1-Ps2 | Cg38-Pg1-Ps3 | Cg38-Pg1-Ps4 |
| Cg38-Pg1-Ps5 | Cg38-Pg1-Ps6 | Cg38-Pg1-Ps7 | Cg38-Pg1-Ps8 |
| Cg38-Pg1-Ps9 | Cg38-Pg1-Ps10 | Cg38-Pg1-Ps11 | Cg38-Pg1-Ps12 |
| Cg38-Pg1-Ps13 | Cg38-Pg1-Ps14 | Cg38-Pg1-Ps15 | Cg38-Pg1-Ps16 |
| Cg38-Pg1-Ps17 | Cg38-Pg1-Ps18 | Cg38-Pg1-Ps19 | Cg38-Pg1-Ps20 |
| Cg38-Pg1-Ps21 | Cg38-Pg1-Ps22 | Cg38-Pg1-Ps23 | Cg38-Pg1-Ps24 |
| Cg38-Pg1-Ps25 | Cg38-Pg1-Ps26 | Cg38-Pg1-Ps27 | Cg38-Pg1-Ps28 |
| Cg38-Pg1-Ps29 | Cg38-Pg1-Ps30 | Cg38-Pg1-Ps31 | Cg38-Pg1-Ps32 |
| Cg38-Pg1-Ps33 | Cg38-Pg1-Ps34 | Cg38-Pg1-Ps35 | Cg38-Pg1-Ps36 |
| Cg38-Pg1-Ps37 | Cg38-Pg1-Ps38 | Cg38-Pg1-Ps39 | Cg38-Pg1-Ps40 |
| Cg38-Pg1-Ps41 | Cg38-Pg1-Ps42 | Cg38-Pg1-Ps43 | Cg38-Pg1-Ps44 |
| Cg38-Pg1-Ps45 | Cg38-Pg1-Ps46 | Cg38-Pg1-Ps47 | Cg38-Pg1-Ps48 |
| Cg38-Pg1-Ps49 | Cg38-Pg1-Ps50 | Cg38-Pg1-Ps51 | Cg38-Pg1-Ps52 |
| Cg38-Pg1-Ps53 | Cg38-Pg1-Ps54 | Cg38-Pg1-Ps55 | Cg38-Pg1-Ps56 |
| Cg38-Pg1-Ps57 | Cg38-Pg1-Ps58 | Cg38-Pg1-Ps59 | Cg38-Pg1-Ps60 |
| Cg38-Pg1-Ps61 | Cg38-Pg1-Ps62 | Cg38-Pg1-Ps63 | Cg38-Pg1-Ps64 |
| Cg38-Pg1-Ps65 | Cg38-Pg1-Ps66 | Cg38-Pg1-Ps67 | Cg38-Pg1-Ps68 |
| Cg38-Pg1-Ps69 | Cg38-Pg1-Ps70 | Cg38-Pg1-Ps71 | Cg38-Pg1-Ps72 |
| Cg38-Pg1-Ps73 | Cg38-Pg1-Ps74 | Cg38-Pg1-Ps75 | Cg38-Pg1-Ps76 |
| Cg38-Pg1-Ps77 | Cg38-Pg1-Ps78 | Cg38-Pg1-Ps79 | Cg38-Pg1-Ps80 |
| Cg38-Pg1-Ps81 | Cg38-Pg1-Ps82 | Cg38-Pg1-Ps83 | Cg38-Pg1-Ps84 |
| Cg38-Pg1-Ps85 | Cg38-Pg1-Ps86 | Cg38-Pg1-Ps87 | Cg38-Pg1-Ps88 |
| Cg38-Pg1-Ps89 | Cg38-Pg1-Ps90 | Cg38-Pg1-Ps91 | Cg38-Pg1-Ps92 |
| Cg38-Pg1-Ps93 | Cg38-Pg1-Ps94 | Cg38-Pg1-Ps95 | Cg38-Pg1-Ps96 |
| Cg38-Pg1-Ps97 | Cg38-Pg1-Ps98 | Cg38-Pg1-Ps99 | Cg38-Pg1-Ps100 |
| Cg38-Pg1-Ps101 | Cg38-Pg1-Ps102 | Cg38-Pg1-Ps103 | Cg38-Pg1-Ps104 |
| Cg38-Pg1-Ps105 | Cg38-Pg1-Ps106 | Cg38-Pg1-Ps107 | Cg38-Pg1-Ps108 |
| Cg38-Pg1-Ps109 | Cg38-Pg1-Ps110 | Cg38-Pg1-Ps111 | Cg38-Pg1-Ps112 |
| Cg38-Pg1-Ps113 | Cg38-Pg1-Ps114 | Cg38-Pg1-Ps115 | Cg38-Pg1-Ps116 |
| Cg38-Pg1-Ps117 | Cg38-Pg1-Ps118 | Cg38-Pg1-Ps119 | Cg38-Pg1-Ps120 |
| Cg38-Pg1-Ps121 | Cg38-Pg1-Ps122 | Cg38-Pg1-Ps123 | Cg38-Pg1-Ps124 |
| Cg38-Pg1-Ps125 | Cg38-Pg1-Ps126 | Cg38-Pg1-Ps127 | Cg38-Pg1-Ps128 |
| Cg38-Pg1-Ps129 | Cg38-Pg1-Ps130 | Cg38-Pg1-Ps131 | Cg38-Pg1-Ps132 |
| Cg38-Pg1-Ps133 | Cg38-Pg1-Ps134 | Cg38-Pg1-Ps135 | Cg38-Pg1-Ps136 |
| Cg38-Pg1-Ps137 | Cg38-Pg1-Ps138 | Cg38-Pg1-Ps139 | Cg38-Pg1-Ps140 |
| Cg38-Pg1-Ps141 | Cg38-Pg1-Ps142 | Cg38-Pg1-Ps143 | Cg38-Pg1-Ps144 |
| Cg38-Pg1-Ps145 | Cg38-Pg1-Ps146 | Cg38-Pg1-Ps147 | Cg38-Pg1-Ps148 |
| Cg38-Pg1-Ps149 | Cg38-Pg1-Ps150 | Cg38-Pg1-Ps151 | Cg38-Pg1-Ps152 |
| Cg38-Pg1-Ps153 | Cg38-Pg1-Ps154 | Cg38-Pg1-Ps155 | Cg38-Pg1-Ps156 |
| Cg38-Pg1-Ps157 | Cg38-Pg1-Ps158 | Cg38-Pg1-Ps159 | Cg38-Pg1-Ps160 |
| Cg38-Pg1-Ps161 | Cg38-Pg1-Ps162 | Cg38-Pg1-Ps163 | Cg38-Pg1-Ps164 |
| Cg38-Pg1-Ps165 | Cg38-Pg1-Ps166 | Cg38-Pg1-Ps167 | Cg38-Pg1-Ps168 |
| Cg38-Pg1-Ps169 | Cg38-Pg1-Ps170 | Cg38-Pg1-Ps171 | Cg38-Pg1-Ps172 |
| Cg38-Pg1-Ps173 | Cg38-Pg1-Ps174 | Cg38-Pg1-Ps175 | Cg38-Pg1-Ps176 |
| Cg38-Pg1-Ps177 | Cg38-Pg1-Ps178 | Cg38-Pg1-Ps179 | Cg38-Pg1-Ps180 |
| Cg38-Pg1-Ps181 | Cg38-Pg1-Ps182 | Cg38-Pg1-Ps183 | Cg38-Pg1-Ps184 |
| Cg38-Pg1-Ps185 | Cg38-Pg1-Ps186 | Cg38-Pg1-Ps187 | Cg38-Pg1-Ps188 |
| Cg38-Pg1-Ps189 | Cg38-Pg1-Ps190 | Cg38-Pg1-Ps191 | Cg38-Pg1-Ps192 |
| Cg38-Pg1-Ps193 | Cg38-Pg1-Ps194 | Cg38-Pg1-Ps195 | Cg38-Pg1-Ps196 |
| Cg38-Pg1-Ps197 | Cg38-Pg1-Ps198 | Cg38-Pg1-Ps199 | Cg38-Pg1-Ps200 |
| Cg38-Pg1-Ps201 | Cg38-Pg1-Ps202 | Cg38-Pg1-Ps203 | Cg38-Pg1-Ps204 |
| Cg38-Pg1-Ps205 | Cg38-Pg1-Ps206 | Cg38-Pg1-Ps207 | Cg38-Pg1-Ps208 |
| Cg38-Pg1-Ps209 | Cg38-Pg1-Ps210 | Cg38-Pg1-Ps211 | Cg38-Pg1-Ps212 |
| Cg38-Pg1-Ps213 | Cg38-Pg1-Ps214 | Cg38-Pg1-Ps215 | Cg38-Pg1-Ps216 |
| Cg38-Pg1-Ps217 | Cg38-Pg1-Ps218 | Cg38-Pg1-Ps219 | Cg38-Pg1-Ps220 |
| Cg38-Pg1-Ps221 | Cg38-Pg1-Ps222 | Cg38-Pg1-Ps223 | Cg38-Pg1-Ps224 |
| Cg38-Pg1-Ps225 | Cg38-Pg1-Ps226 | Cg38-Pg1-Ps227 | Cg38-Pg1-Ps228 |
| Cg38-Pg1-Ps229 | Cg38-Pg1-Ps230 | Cg38-Pg1-Ps231 | Cg38-Pg1-Ps232 |
| Cg38-Pg1-Ps233 | Cg38-Pg1-Ps234 | Cg38-Pg1-Ps235 | Cg38-Pg1-Ps236 |
| Cg38-Pg1-Ps237 | Cg38-Pg1-Ps238 | Cg38-Pg1-Ps239 | Cg38-Pg1-Ps240 |
| Cg38-Pg1-Ps241 | Cg38-Pg1-Ps242 | Cg38-Pg1-Ps243 | |
| Cg39-Pg1-Ps1 | Cg39-Pg1-Ps2 | Cg39-Pg1-Ps3 | Cg39-Pg1-Ps4 |
| Cg39-Pg1-Ps5 | Cg39-Pg1-Ps6 | Cg39-Pg1-Ps7 | Cg39-Pg1-Ps8 |
| Cg39-Pg1-Ps9 | Cg39-Pg1-Ps10 | Cg39-Pg1-Ps11 | Cg39-Pg1-Ps12 |
| Cg39-Pg1-Ps13 | Cg39-Pg1-Ps14 | Cg39-Pg1-Ps15 | Cg39-Pg1-Ps16 |
| Cg39-Pg1-Ps17 | Cg39-Pg1-Ps18 | Cg39-Pg1-Ps19 | Cg39-Pg1-Ps20 |
| Cg39-Pg1-Ps21 | Cg39-Pg1-Ps22 | Cg39-Pg1-Ps23 | Cg39-Pg1-Ps24 |
| Cg39-Pg1-Ps25 | Cg39-Pg1-Ps26 | Cg39-Pg1-Ps27 | Cg39-Pg1-Ps28 |
| Cg39-Pg1-Ps29 | Cg39-Pg1-Ps30 | Cg39-Pg1-Ps31 | Cg39-Pg1-Ps32 |
| Cg39-Pg1-Ps33 | Cg39-Pg1-Ps34 | Cg39-Pg1-Ps35 | Cg39-Pg1-Ps36 |
| Cg39-Pg1-Ps37 | Cg39-Pg1-Ps38 | Cg39-Pg1-Ps39 | Cg39-Pg1-Ps40 |
| Cg39-Pg1-Ps41 | Cg39-Pg1-Ps42 | Cg39-Pg1-Ps43 | Cg39-Pg1-Ps44 |
| Cg39-Pg1-Ps45 | Cg39-Pg1-Ps46 | Cg39-Pg1-Ps47 | Cg39-Pg1-Ps48 |
| Cg39-Pg1-Ps49 | Cg39-Pg1-Ps50 | Cg39-Pg1-Ps51 | Cg39-Pg1-Ps52 |
| Cg39-Pg1-Ps53 | Cg39-Pg1-Ps54 | Cg39-Pg1-Ps55 | Cg39-Pg1-Ps56 |
| Cg39-Pg1-Ps57 | Cg39-Pg1-Ps58 | Cg39-Pg1-Ps59 | Cg39-Pg1-Ps60 |
| Cg39-Pg1-Ps61 | Cg39-Pg1-Ps62 | Cg39-Pg1-Ps63 | Cg39-Pg1-Ps64 |
| Cg39-Pg1-Ps65 | Cg39-Pg1-Ps66 | Cg39-Pg1-Ps67 | Cg39-Pg1-Ps68 |
| Cg39-Pg1-Ps69 | Cg39-Pg1-Ps70 | Cg39-Pg1-Ps71 | Cg39-Pg1-Ps72 |
| Cg39-Pg1-Ps73 | Cg39-Pg1-Ps74 | Cg39-Pg1-Ps75 | Cg39-Pg1-Ps76 |
| Cg39-Pg1-Ps77 | Cg39-Pg1-Ps78 | Cg39-Pg1-Ps79 | Cg39-Pg1-Ps80 |
| Cg39-Pg1-Ps81 | Cg39-Pg1-Ps82 | Cg39-Pg1-Ps83 | Cg39-Pg1-Ps84 |
| Cg39-Pg1-Ps85 | Cg39-Pg1-Ps86 | Cg39-Pg1-Ps87 | Cg39-Pg1-Ps88 |
| Cg39-Pg1-Ps89 | Cg39-Pg1-Ps90 | Cg39-Pg1-Ps91 | Cg39-Pg1-Ps92 |
| Cg39-Pg1-Ps93 | Cg39-Pg1-Ps94 | Cg39-Pg1-Ps95 | Cg39-Pg1-Ps96 |
| Cg39-Pg1-Ps97 | Cg39-Pg1-Ps98 | Cg39-Pg1-Ps99 | Cg39-Pg1-Ps100 |
| Cg39-Pg1-Ps101 | Cg39-Pg1-Ps102 | Cg39-Pg1-Ps103 | Cg39-Pg1-Ps104 |
| Cg39-Pg1-Ps105 | Cg39-Pg1-Ps106 | Cg39-Pg1-Ps107 | Cg39-Pg1-Ps108 |
| Cg39-Pg1-Ps109 | Cg39-Pg1-Ps110 | Cg39-Pg1-Ps111 | Cg39-Pg1-Ps112 |
| Cg39-Pg1-Ps113 | Cg39-Pg1-Ps114 | Cg39-Pg1-Ps115 | Cg39-Pg1-Ps116 |
| Cg39-Pg1-Ps117 | Cg39-Pg1-Ps118 | Cg39-Pg1-Ps119 | Cg39-Pg1-Ps120 |
| Cg39-Pg1-Ps121 | Cg39-Pg1-Ps122 | Cg39-Pg1-Ps123 | Cg39-Pg1-Ps124 |
| Cg39-Pg1-Ps125 | Cg39-Pg1-Ps126 | Cg39-Pg1-Ps127 | Cg39-Pg1-Ps128 |
| Cg39-Pg1-Ps129 | Cg39-Pg1-Ps130 | Cg39-Pg1-Ps131 | Cg39-Pg1-Ps132 |
| Cg39-Pg1-Ps133 | Cg39-Pg1-Ps134 | Cg39-Pg1-Ps135 | Cg39-Pg1-Ps136 |
| Cg39-Pg1-Ps137 | Cg39-Pg1-Ps138 | Cg39-Pg1-Ps139 | Cg39-Pg1-Ps140 |
| Cg39-Pg1-Ps141 | Cg39-Pg1-Ps142 | Cg39-Pg1-Ps143 | Cg39-Pg1-Ps144 |
| Cg39-Pg1-Ps145 | Cg39-Pg1-Ps146 | Cg39-Pg1-Ps147 | Cg39-Pg1-Ps148 |
| Cg39-Pg1-Ps149 | Cg39-Pg1-Ps150 | Cg39-Pg1-Ps151 | Cg39-Pg1-Ps152 |
| Cg39-Pg1-Ps153 | Cg39-Pg1-Ps154 | Cg39-Pg1-Ps155 | Cg39-Pg1-Ps156 |
| Cg39-Pg1-Ps157 | Cg39-Pg1-Ps158 | Cg39-Pg1-Ps159 | Cg39-Pg1-Ps160 |
| Cg39-Pg1-Ps161 | Cg39-Pg1-Ps162 | Cg39-Pg1-Ps163 | Cg39-Pg1-Ps164 |
| Cg39-Pg1-Ps165 | Cg39-Pg1-Ps166 | Cg39-Pg1-Ps167 | Cg39-Pg1-Ps168 |
| Cg39-Pg1-Ps169 | Cg39-Pg1-Ps170 | Cg39-Pg1-Ps171 | Cg39-Pg1-Ps172 |
| Cg39-Pg1-Ps173 | Cg39-Pg1-Ps174 | Cg39-Pg1-Ps175 | Cg39-Pg1-Ps176 |
| Cg39-Pg1-Ps177 | Cg39-Pg1-Ps178 | Cg39-Pg1-Ps179 | Cg39-Pg1-Ps180 |
| Cg39-Pg1-Ps181 | Cg39-Pg1-Ps182 | Cg39-Pg1-Ps183 | Cg39-Pg1-Ps184 |
| Cg39-Pg1-Ps185 | Cg39-Pg1-Ps186 | Cg39-Pg1-Ps187 | Cg39-Pg1-Ps188 |
| Cg39-Pg1-Ps189 | Cg39-Pg1-Ps190 | Cg39-Pg1-Ps191 | Cg39-Pg1-Ps192 |
| Cg39-Pg1-Ps193 | Cg39-Pg1-Ps194 | Cg39-Pg1-Ps195 | Cg39-Pg1-Ps196 |
| Cg39-Pg1-Ps197 | Cg39-Pg1-Ps198 | Cg39-Pg1-Ps199 | Cg39-Pg1-Ps200 |
| Cg39-Pg1-Ps201 | Cg39-Pg1-Ps202 | Cg39-Pg1-Ps203 | Cg39-Pg1-Ps204 |
| Cg39-Pg1-Ps205 | Cg39-Pg1-Ps206 | Cg39-Pg1-Ps207 | Cg39-Pg1-Ps208 |
| Cg39-Pg1-Ps209 | Cg39-Pg1-Ps210 | Cg39-Pg1-Ps211 | Cg39-Pg1-Ps212 |
| Cg39-Pg1-Ps213 | Cg39-Pg1-Ps214 | Cg39-Pg1-Ps215 | Cg39-Pg1-Ps216 |
| Cg39-Pg1-Ps217 | Cg39-Pg1-Ps218 | Cg39-Pg1-Ps219 | Cg39-Pg1-Ps220 |
| Cg39-Pg1-Ps221 | Cg39-Pg1-Ps222 | Cg39-Pg1-Ps223 | Cg39-Pg1-Ps224 |
| Cg39-Pg1-Ps225 | Cg39-Pg1-Ps226 | Cg39-Pg1-Ps227 | Cg39-Pg1-Ps228 |
| Cg39-Pg1-Ps229 | Cg39-Pg1-Ps230 | Cg39-Pg1-Ps231 | Cg39-Pg1-Ps232 |
| Cg39-Pg1-Ps233 | Cg39-Pg1-Ps234 | Cg39-Pg1-Ps235 | Cg39-Pg1-Ps236 |
| Cg39-Pg1-Ps237 | Cg39-Pg1-Ps238 | Cg39-Pg1-Ps239 | Cg39-Pg1-Ps240 |
| Cg39-Pg1-Ps241 | Cg39-Pg1-Ps242 | Cg39-Pg1-Ps243 | |
| Cg41-Pg1-Ps1 | Cg41-Pg1-Ps2 | Cg41-Pg1-Ps3 | Cg41-Pg1-Ps4 |
| Cg41-Pg1-Ps5 | Cg41-Pg1-Ps6 | Cg41-Pg1-Ps7 | Cg41-Pg1-Ps8 |
| Cg41-Pg1-Ps9 | Cg41-Pg1-Ps10 | Cg41-Pg1-Ps11 | Cg41-Pg1-Ps12 |
| Cg41-Pg1-Ps13 | Cg41-Pg1-Ps14 | Cg41-Pg1-Ps15 | Cg41-Pg1-Ps16 |
| Cg41-Pg1-Ps17 | Cg41-Pg1-Ps18 | Cg41-Pg1-Ps19 | Cg41-Pg1-Ps20 |
| Cg41-Pg1-Ps21 | Cg41-Pg1-Ps22 | Cg41-Pg1-Ps23 | Cg41-Pg1-Ps24 |
| Cg41-Pg1-Ps25 | Cg41-Pg1-Ps26 | Cg41-Pg1-Ps27 | Cg41-Pg1-Ps28 |
| Cg41-Pg1-Ps29 | Cg41-Pg1-Ps30 | Cg41-Pg1-Ps31 | Cg41-Pg1-Ps32 |
| Cg41-Pg1-Ps33 | Cg41-Pg1-Ps34 | Cg41-Pg1-Ps35 | Cg41-Pg1-Ps36 |
| Cg41-Pg1-Ps37 | Cg41-Pg1-Ps38 | Cg41-Pg1-Ps39 | Cg41-Pg1-Ps40 |
| Cg41-Pg1-Ps41 | Cg41-Pg1-Ps42 | Cg41-Pg1-Ps43 | Cg41-Pg1-Ps44 |
| Cg41-Pg1-Ps45 | Cg41-Pg1-Ps46 | Cg41-Pg1-Ps47 | Cg41-Pg1-Ps48 |
| Cg41-Pg1-Ps49 | Cg41-Pg1-Ps50 | Cg41-Pg1-Ps51 | Cg41-Pg1-Ps52 |
| Cg41-Pg1-Ps53 | Cg41-Pg1-Ps54 | Cg41-Pg1-Ps55 | Cg41-Pg1-Ps56 |
| Cg41-Pg1-Ps57 | Cg41-Pg1-Ps58 | Cg41-Pg1-Ps59 | Cg41-Pg1-Ps60 |
| Cg41-Pg1-Ps61 | Cg41-Pg1-Ps62 | Cg41-Pg1-Ps63 | Cg41-Pg1-Ps64 |
| Cg41-Pg1-Ps65 | Cg41-Pg1-Ps66 | Cg41-Pg1-Ps67 | Cg41-Pg1-Ps68 |
| Cg41-Pg1-Ps69 | Cg41-Pg1-Ps70 | Cg41-Pg1-Ps71 | Cg41-Pg1-Ps72 |
| Cg41-Pg1-Ps73 | Cg41-Pg1-Ps74 | Cg41-Pg1-Ps75 | Cg41-Pg1-Ps76 |
| Cg41-Pg1-Ps77 | Cg41-Pg1-Ps78 | Cg41-Pg1-Ps79 | Cg41-Pg1-Ps80 |
| Cg41-Pg1-Ps81 | Cg41-Pg1-Ps82 | Cg41-Pg1-Ps83 | Cg41-Pg1-Ps84 |
| Cg41-Pg1-Ps85 | Cg41-Pg1-Ps86 | Cg41-Pg1-Ps87 | Cg41-Pg1-Ps88 |
| Cg41-Pg1-Ps89 | Cg41-Pg1-Ps90 | Cg41-Pg1-Ps91 | Cg41-Pg1-Ps92 |
| Cg41-Pg1-Ps93 | Cg41-Pg1-Ps94 | Cg41-Pg1-Ps95 | Cg41-Pg1-Ps96 |
| Cg41-Pg1-Ps97 | Cg41-Pg1-Ps98 | Cg41-Pg1-Ps99 | Cg41-Pg1-Ps100 |
| Cg41-Pg1-Ps101 | Cg41-Pg1-Ps102 | Cg41-Pg1-Ps103 | Cg41-Pg1-Ps104 |
| Cg41-Pg1-Ps105 | Cg41-Pg1-Ps106 | Cg41-Pg1-Ps107 | Cg41-Pg1-Ps108 |
| Cg41-Pg1-Ps109 | Cg41-Pg1-Ps110 | Cg41-Pg1-Ps111 | Cg41-Pg1-Ps112 |
| Cg41-Pg1-Ps113 | Cg41-Pg1-Ps114 | Cg41-Pg1-Ps115 | Cg41-Pg1-Ps116 |
| Cg41-Pg1-Ps117 | Cg41-Pg1-Ps118 | Cg41-Pg1-Ps119 | Cg41-Pg1-Ps120 |
| Cg41-Pg1-Ps121 | Cg41-Pg1-Ps122 | Cg41-Pg1-Ps123 | Cg41-Pg1-Ps124 |
| Cg41-Pg1-Ps125 | Cg41-Pg1-Ps126 | Cg41-Pg1-Ps127 | Cg41-Pg1-Ps128 |
| Cg41-Pg1-Ps129 | Cg41-Pg1-Ps130 | Cg41-Pg1-Ps131 | Cg41-Pg1-Ps132 |
| Cg41-Pg1-Ps133 | Cg41-Pg1-Ps134 | Cg41-Pg1-Ps135 | Cg41-Pg1-Ps136 |
| Cg41-Pg1-Ps137 | Cg41-Pg1-Ps138 | Cg41-Pg1-Ps139 | Cg41-Pg1-Ps140 |
| Cg41-Pg1-Ps141 | Cg41-Pg1-Ps142 | Cg41-Pg1-Ps143 | Cg41-Pg1-Ps144 |
| Cg41-Pg1-Ps145 | Cg41-Pg1-Ps146 | Cg41-Pg1-Ps147 | Cg41-Pg1-Ps148 |
| Cg41-Pg1-Ps149 | Cg41-Pg1-Ps150 | Cg41-Pg1-Ps151 | Cg41-Pg1-Ps152 |
| Cg41-Pg1-Ps153 | Cg41-Pg1-Ps154 | Cg41-Pg1-Ps155 | Cg41-Pg1-Ps156 |
| Cg41-Pg1-Ps157 | Cg41-Pg1-Ps158 | Cg41-Pg1-Ps159 | Cg41-Pg1-Ps160 |
| Cg41-Pg1-Ps161 | Cg41-Pg1-Ps162 | Cg41-Pg1-Ps163 | Cg41-Pg1-Ps164 |
| Cg41-Pg1-Ps165 | Cg41-Pg1-Ps166 | Cg41-Pg1-Ps167 | Cg41-Pg1-Ps168 |
| Cg41-Pg1-Ps169 | Cg41-Pg1-Ps170 | Cg41-Pg1-Ps171 | Cg41-Pg1-Ps172 |
| Cg41-Pg1-Ps173 | Cg41-Pg1-Ps174 | Cg41-Pg1-Ps175 | Cg41-Pg1-Ps176 |
| Cg41-Pg1-Ps177 | Cg41-Pg1-Ps178 | Cg41-Pg1-Ps179 | Cg41-Pg1-Ps180 |
| Cg41-Pg1-Ps181 | Cg41-Pg1-Ps182 | Cg41-Pg1-Ps183 | Cg41-Pg1-Ps184 |
| Cg41-Pg1-Ps185 | Cg41-Pg1-Ps186 | Cg41-Pg1-Ps187 | Cg41-Pg1-Ps188 |
| Cg41-Pg1-Ps189 | Cg41-Pg1-Ps190 | Cg41-Pg1-Ps191 | Cg41-Pg1-Ps192 |
| Cg41-Pg1-Ps193 | Cg41-Pg1-Ps194 | Cg41-Pg1-Ps195 | Cg41-Pg1-Ps196 |
| Cg41-Pg1-Ps197 | Cg41-Pg1-Ps198 | Cg41-Pg1-Ps199 | Cg41-Pg1-Ps200 |
| Cg41-Pg1-Ps201 | Cg41-Pg1-Ps202 | Cg41-Pg1-Ps203 | Cg41-Pg1-Ps204 |
| Cg41-Pg1-Ps205 | Cg41-Pg1-Ps206 | Cg41-Pg1-Ps207 | Cg41-Pg1-Ps208 |
| Cg41-Pg1-Ps209 | Cg41-Pg1-Ps210 | Cg41-Pg1-Ps211 | Cg41-Pg1-Ps212 |
| Cg41-Pg1-Ps213 | Cg41-Pg1-Ps214 | Cg41-Pg1-Ps215 | Cg41-Pg1-Ps216 |
| Cg41-Pg1-Ps217 | Cg41-Pg1-Ps218 | Cg41-Pg1-Ps219 | Cg41-Pg1-Ps220 |
| Cg41-Pg1-Ps221 | Cg41-Pg1-Ps222 | Cg41-Pg1-Ps223 | Cg41-Pg1-Ps224 |
| Cg41-Pg1-Ps225 | Cg41-Pg1-Ps226 | Cg41-Pg1-Ps227 | Cg41-Pg1-Ps228 |
| Cg41-Pg1-Ps229 | Cg41-Pg1-Ps230 | Cg41-Pg1-Ps231 | Cg41-Pg1-Ps232 |
| Cg41-Pg1-Ps233 | Cg41-Pg1-Ps234 | Cg41-Pg1-Ps235 | Cg41-Pg1-Ps236 |
| Cg41-Pg1-Ps237 | Cg41-Pg1-Ps238 | Cg41-Pg1-Ps239 | Cg41-Pg1-Ps240 |
| Cg41-Pg1-Ps241 | Cg41-Pg1-Ps242 | Cg41-Pg1-Ps243 | |
| Cg42-Pg1-Ps1 | Cg42-Pg1-Ps2 | Cg42-Pg1-Ps3 | Cg42-Pg1-Ps4 |
| Cg42-Pg1-Ps5 | Cg42-Pg1-Ps6 | Cg42-Pg1-Ps7 | Cg42-Pg1-Ps8 |
| Cg42-Pg1-Ps9 | Cg42-Pg1-Ps10 | Cg42-Pg1-Ps11 | Cg42-Pg1-Ps12 |
| Cg42-Pg1-Ps13 | Cg42-Pg1-Ps14 | Cg42-Pg1-Ps15 | Cg42-Pg1-Ps16 |
| Cg42-Pg1-Ps17 | Cg42-Pg1-Ps18 | Cg42-Pg1-Ps19 | Cg42-Pg1-Ps20 |
| Cg42-Pg1-Ps21 | Cg42-Pg1-Ps22 | Cg42-Pg1-Ps23 | Cg42-Pg1-Ps24 |
| Cg42-Pg1-Ps25 | Cg42-Pg1-Ps26 | Cg42-Pg1-Ps27 | Cg42-Pg1-Ps28 |
| Cg42-Pg1-Ps29 | Cg42-Pg1-Ps30 | Cg42-Pg1-Ps31 | Cg42-Pg1-Ps32 |
| Cg42-Pg1-Ps33 | Cg42-Pg1-Ps34 | Cg42-Pg1-Ps35 | Cg42-Pg1-Ps36 |
| Cg42-Pg1-Ps37 | Cg42-Pg1-Ps38 | Cg42-Pg1-Ps39 | Cg42-Pg1-Ps40 |
| Cg42-Pg1-Ps41 | Cg42-Pg1-Ps42 | Cg42-Pg1-Ps43 | Cg42-Pg1-Ps44 |
| Cg42-Pg1-Ps45 | Cg42-Pg1-Ps46 | Cg42-Pg1-Ps47 | Cg42-Pg1-Ps48 |
| Cg42-Pg1-Ps49 | Cg42-Pg1-Ps50 | Cg42-Pg1-Ps51 | Cg42-Pg1-Ps52 |
| Cg42-Pg1-Ps53 | Cg42-Pg1-Ps54 | Cg42-Pg1-Ps55 | Cg42-Pg1-Ps56 |
| Cg42-Pg1-Ps57 | Cg42-Pg1-Ps58 | Cg42-Pg1-Ps59 | Cg42-Pg1-Ps60 |
| Cg42-Pg1-Ps61 | Cg42-Pg1-Ps62 | Cg42-Pg1-Ps63 | Cg42-Pg1-Ps64 |
| Cg42-Pg1-Ps65 | Cg42-Pg1-Ps66 | Cg42-Pg1-Ps67 | Cg42-Pg1-Ps68 |
| Cg42-Pg1-Ps69 | Cg42-Pg1-Ps70 | Cg42-Pg1-Ps71 | Cg42-Pg1-Ps72 |
| Cg42-Pg1-Ps73 | Cg42-Pg1-Ps74 | Cg42-Pg1-Ps75 | Cg42-Pg1-Ps76 |
| Cg42-Pg1-Ps77 | Cg42-Pg1-Ps78 | Cg42-Pg1-Ps79 | Cg42-Pg1-Ps80 |
| Cg42-Pg1-Ps81 | Cg42-Pg1-Ps82 | Cg42-Pg1-Ps83 | Cg42-Pg1-Ps84 |
| Cg42-Pg1-Ps85 | Cg42-Pg1-Ps86 | Cg42-Pg1-Ps87 | Cg42-Pg1-Ps88 |
| Cg42-Pg1-Ps89 | Cg42-Pg1-Ps90 | Cg42-Pg1-Ps91 | Cg42-Pg1-Ps92 |
| Cg42-Pg1-Ps93 | Cg42-Pg1-Ps94 | Cg42-Pg1-Ps95 | Cg42-Pg1-Ps96 |
| Cg42-Pg1-Ps97 | Cg42-Pg1-Ps98 | Cg42-Pg1-Ps99 | Cg42-Pg1-Ps100 |
| Cg42-Pg1-Ps101 | Cg42-Pg1-Ps102 | Cg42-Pg1-Ps103 | Cg42-Pg1-Ps104 |
| Cg42-Pg1-Ps105 | Cg42-Pg1-Ps106 | Cg42-Pg1-Ps107 | Cg42-Pg1-Ps108 |
| Cg42-Pg1-Ps109 | Cg42-Pg1-Ps110 | Cg42-Pg1-Ps111 | Cg42-Pg1-Ps112 |
| Cg42-Pg1-Ps113 | Cg42-Pg1-Ps114 | Cg42-Pg1-Ps115 | Cg42-Pg1-Ps116 |
| Cg42-Pg1-Ps117 | Cg42-Pg1-Ps118 | Cg42-Pg1-Ps119 | Cg42-Pg1-Ps120 |
| Cg42-Pg1-Ps121 | Cg42-Pg1-Ps122 | Cg42-Pg1-Ps123 | Cg42-Pg1-Ps124 |
| Cg42-Pg1-Ps125 | Cg42-Pg1-Ps126 | Cg42-Pg1-Ps127 | Cg42-Pg1-Ps128 |
| Cg42-Pg1-Ps129 | Cg42-Pg1-Ps130 | Cg42-Pg1-Ps131 | Cg42-Pg1-Ps132 |
| Cg42-Pg1-Ps133 | Cg42-Pg1-Ps134 | Cg42-Pg1-Ps135 | Cg42-Pg1-Ps136 |
| Cg42-Pg1-Ps137 | Cg42-Pg1-Ps138 | Cg42-Pg1-Ps139 | Cg42-Pg1-Ps140 |
| Cg42-Pg1-Ps141 | Cg42-Pg1-Ps142 | Cg42-Pg1-Ps143 | Cg42-Pg1-Ps144 |
| Cg42-Pg1-Ps145 | Cg42-Pg1-Ps146 | Cg42-Pg1-Ps147 | Cg42-Pg1-Ps148 |
| Cg42-Pg1-Ps149 | Cg42-Pg1-Ps150 | Cg42-Pg1-Ps151 | Cg42-Pg1-Ps152 |
| Cg42-Pg1-Ps153 | Cg42-Pg1-Ps154 | Cg42-Pg1-Ps155 | Cg42-Pg1-Ps156 |
| Cg42-Pg1-Ps157 | Cg42-Pg1-Ps158 | Cg42-Pg1-Ps159 | Cg42-Pg1-Ps160 |
| Cg42-Pg1-Ps161 | Cg42-Pg1-Ps162 | Cg42-Pg1-Ps163 | Cg42-Pg1-Ps164 |
| Cg42-Pg1-Ps165 | Cg42-Pg1-Ps166 | Cg42-Pg1-Ps167 | Cg42-Pg1-Ps168 |
| Cg42-Pg1-Ps169 | Cg42-Pg1-Ps170 | Cg42-Pg1-Ps171 | Cg42-Pg1-Ps172 |
| Cg42-Pg1-Ps173 | Cg42-Pg1-Ps174 | Cg42-Pg1-Ps175 | Cg42-Pg1-Ps176 |
| Cg42-Pg1-Ps177 | Cg42-Pg1-Ps178 | Cg42-Pg1-Ps179 | Cg42-Pg1-Ps180 |
| Cg42-Pg1-Ps181 | Cg42-Pg1-Ps182 | Cg42-Pg1-Ps183 | Cg42-Pg1-Ps184 |
| Cg42-Pg1-Ps185 | Cg42-Pg1-Ps186 | Cg42-Pg1-Ps187 | Cg42-Pg1-Ps188 |
| Cg42-Pg1-Ps189 | Cg42-Pg1-Ps190 | Cg42-Pg1-Ps191 | Cg42-Pg1-Ps192 |
| Cg42-Pg1-Ps193 | Cg42-Pg1-Ps194 | Cg42-Pg1-Ps195 | Cg42-Pg1-Ps196 |
| Cg42-Pg1-Ps197 | Cg42-Pg1-Ps198 | Cg42-Pg1-Ps199 | Cg42-Pg1-Ps200 |
| Cg42-Pg1-Ps201 | Cg42-Pg1-Ps202 | Cg42-Pg1-Ps203 | Cg42-Pg1-Ps204 |
| Cg42-Pg1-Ps205 | Cg42-Pg1-Ps206 | Cg42-Pg1-Ps207 | Cg42-Pg1-Ps208 |
| Cg42-Pg1-Ps209 | Cg42-Pg1-Ps210 | Cg42-Pg1-Ps211 | Cg42-Pg1-Ps212 |
| Cg42-Pg1-Ps213 | Cg42-Pg1-Ps214 | Cg42-Pg1-Ps215 | Cg42-Pg1-Ps216 |
| Cg42-Pg1-Ps217 | Cg42-Pg1-Ps218 | Cg42-Pg1-Ps219 | Cg42-Pg1-Ps220 |
| Cg42-Pg1-Ps221 | Cg42-Pg1-Ps222 | Cg42-Pg1-Ps223 | Cg42-Pg1-Ps224 |
| Cg42-Pg1-Ps225 | Cg42-Pg1-Ps226 | Cg42-Pg1-Ps227 | Cg42-Pg1-Ps228 |
| Cg42-Pg1-Ps229 | Cg42-Pg1-Ps230 | Cg42-Pg1-Ps231 | Cg42-Pg1-Ps232 |
| Cg42-Pg1-Ps233 | Cg42-Pg1-Ps234 | Cg42-Pg1-Ps235 | Cg42-Pg1-Ps236 |
| Cg42-Pg1-Ps237 | Cg42-Pg1-Ps238 | Cg42-Pg1-Ps239 | Cg42-Pg1-Ps240 |
| Cg42-Pg1-Ps241 | Cg42-Pg1-Ps242 | Cg42-Pg1-Ps243 | |
| Cg44-Pg1-Ps1 | Cg44-Pg1-Ps2 | Cg44-Pg1-Ps3 | Cg44-Pg1-Ps4 |
| Cg44-Pg1-Ps5 | Cg44-Pg1-Ps6 | Cg44-Pg1-Ps7 | Cg44-Pg1-Ps8 |
| Cg44-Pg1-Ps9 | Cg44-Pg1-Ps10 | Cg44-Pg1-Ps11 | Cg44-Pg1-Ps12 |
| Cg44-Pg1-Ps13 | Cg44-Pg1-Ps14 | Cg44-Pg1-Ps15 | Cg44-Pg1-Ps16 |
| Cg44-Pg1-Ps17 | Cg44-Pg1-Ps18 | Cg44-Pg1-Ps19 | Cg44-Pg1-Ps20 |
| Cg44-Pg1-Ps21 | Cg44-Pg1-Ps22 | Cg44-Pg1-Ps23 | Cg44-Pg1-Ps24 |
| Cg44-Pg1-Ps25 | Cg44-Pg1-Ps26 | Cg44-Pg1-Ps27 | Cg44-Pg1-Ps28 |
| Cg44-Pg1-Ps29 | Cg44-Pg1-Ps30 | Cg44-Pg1-Ps31 | Cg44-Pg1-Ps32 |
| Cg44-Pg1-Ps33 | Cg44-Pg1-Ps34 | Cg44-Pg1-Ps35 | Cg44-Pg1-Ps36 |
| Cg44-Pg1-Ps37 | Cg44-Pg1-Ps38 | Cg44-Pg1-Ps39 | Cg44-Pg1-Ps40 |
| Cg44-Pg1-Ps41 | Cg44-Pg1-Ps42 | Cg44-Pg1-Ps43 | Cg44-Pg1-Ps44 |
| Cg44-Pg1-Ps45 | Cg44-Pg1-Ps46 | Cg44-Pg1-Ps47 | Cg44-Pg1-Ps48 |
| Cg44-Pg1-Ps49 | Cg44-Pg1-Ps50 | Cg44-Pg1-Ps51 | Cg44-Pg1-Ps52 |
| Cg44-Pg1-Ps53 | Cg44-Pg1-Ps54 | Cg44-Pg1-Ps55 | Cg44-Pg1-Ps56 |
| Cg44-Pg1-Ps57 | Cg44-Pg1-Ps58 | Cg44-Pg1-Ps59 | Cg44-Pg1-Ps60 |
| Cg44-Pg1-Ps61 | Cg44-Pg1-Ps62 | Cg44-Pg1-Ps63 | Cg44-Pg1-Ps64 |
| Cg44-Pg1-Ps65 | Cg44-Pg1-Ps66 | Cg44-Pg1-Ps67 | Cg44-Pg1-Ps68 |
| Cg44-Pg1-Ps69 | Cg44-Pg1-Ps70 | Cg44-Pg1-Ps71 | Cg44-Pg1-Ps72 |
| Cg44-Pg1-Ps73 | Cg44-Pg1-Ps74 | Cg44-Pg1-Ps75 | Cg44-Pg1-Ps76 |
| Cg44-Pg1-Ps77 | Cg44-Pg1-Ps78 | Cg44-Pg1-Ps79 | Cg44-Pg1-Ps80 |
| Cg44-Pg1-Ps81 | Cg44-Pg1-Ps82 | Cg44-Pg1-Ps83 | Cg44-Pg1-Ps84 |
| Cg44-Pg1-Ps85 | Cg44-Pg1-Ps86 | Cg44-Pg1-Ps87 | Cg44-Pg1-Ps88 |
| Cg44-Pg1-Ps89 | Cg44-Pg1-Ps90 | Cg44-Pg1-Ps91 | Cg44-Pg1-Ps92 |
| Cg44-Pg1-Ps93 | Cg44-Pg1-Ps94 | Cg44-Pg1-Ps95 | Cg44-Pg1-Ps96 |
| Cg44-Pg1-Ps97 | Cg44-Pg1-Ps98 | Cg44-Pg1-Ps99 | Cg44-Pg1-Ps100 |
| Cg44-Pg1-Ps101 | Cg44-Pg1-Ps102 | Cg44-Pg1-Ps103 | Cg44-Pg1-Ps104 |
| Cg44-Pg1-Ps105 | Cg44-Pg1-Ps106 | Cg44-Pg1-Ps107 | Cg44-Pg1-Ps108 |
| Cg44-Pg1-Ps109 | Cg44-Pg1-Ps110 | Cg44-Pg1-Ps111 | Cg44-Pg1-Ps112 |
| Cg44-Pg1-Ps113 | Cg44-Pg1-Ps114 | Cg44-Pg1-Ps115 | Cg44-Pg1-Ps116 |
| Cg44-Pg1-Ps117 | Cg44-Pg1-Ps118 | Cg44-Pg1-Ps119 | Cg44-Pg1-Ps120 |
| Cg44-Pg1-Ps121 | Cg44-Pg1-Ps122 | Cg44-Pg1-Ps123 | Cg44-Pg1-Ps124 |
| Cg44-Pg1-Ps125 | Cg44-Pg1-Ps126 | Cg44-Pg1-Ps127 | Cg44-Pg1-Ps128 |
| Cg44-Pg1-Ps129 | Cg44-Pg1-Ps130 | Cg44-Pg1-Ps131 | Cg44-Pg1-Ps132 |
| Cg44-Pg1-Ps133 | Cg44-Pg1-Ps134 | Cg44-Pg1-Ps135 | Cg44-Pg1-Ps136 |
| Cg44-Pg1-Ps137 | Cg44-Pg1-Ps138 | Cg44-Pg1-Ps139 | Cg44-Pg1-Ps140 |
| Cg44-Pg1-Ps141 | Cg44-Pg1-Ps142 | Cg44-Pg1-Ps143 | Cg44-Pg1-Ps144 |
| Cg44-Pg1-Ps145 | Cg44-Pg1-Ps146 | Cg44-Pg1-Ps147 | Cg44-Pg1-Ps148 |
| Cg44-Pg1-Ps149 | Cg44-Pg1-Ps150 | Cg44-Pg1-Ps151 | Cg44-Pg1-Ps152 |
| Cg44-Pg1-Ps153 | Cg44-Pg1-Ps154 | Cg44-Pg1-Ps155 | Cg44-Pg1-Ps156 |
| Cg44-Pg1-Ps157 | Cg44-Pg1-Ps158 | Cg44-Pg1-Ps159 | Cg44-Pg1-Ps160 |
| Cg44-Pg1-Ps161 | Cg44-Pg1-Ps162 | Cg44-Pg1-Ps163 | Cg44-Pg1-Ps164 |
| Cg44-Pg1-Ps165 | Cg44-Pg1-Ps166 | Cg44-Pg1-Ps167 | Cg44-Pg1-Ps168 |
| Cg44-Pg1-Ps169 | Cg44-Pg1-Ps170 | Cg44-Pg1-Ps171 | Cg44-Pg1-Ps172 |
| Cg44-Pg1-Ps173 | Cg44-Pg1-Ps174 | Cg44-Pg1-Ps175 | Cg44-Pg1-Ps176 |
| Cg44-Pg1-Ps177 | Cg44-Pg1-Ps178 | Cg44-Pg1-Ps179 | Cg44-Pg1-Ps180 |
| Cg44-Pg1-Ps181 | Cg44-Pg1-Ps182 | Cg44-Pg1-Ps183 | Cg44-Pg1-Ps184 |
| Cg44-Pg1-Ps185 | Cg44-Pg1-Ps186 | Cg44-Pg1-Ps187 | Cg44-Pg1-Ps188 |
| Cg44-Pg1-Ps189 | Cg44-Pg1-Ps190 | Cg44-Pg1-Ps191 | Cg44-Pg1-Ps192 |
| Cg44-Pg1-Ps193 | Cg44-Pg1-Ps194 | Cg44-Pg1-Ps195 | Cg44-Pg1-Ps196 |
| Cg44-Pg1-Ps197 | Cg44-Pg1-Ps198 | Cg44-Pg1-Ps199 | Cg44-Pg1-Ps200 |
| Cg44-Pg1-Ps201 | Cg44-Pg1-Ps202 | Cg44-Pg1-Ps203 | Cg44-Pg1-Ps204 |
| Cg44-Pg1-Ps205 | Cg44-Pg1-Ps206 | Cg44-Pg1-Ps207 | Cg44-Pg1-Ps208 |
| Cg44-Pg1-Ps209 | Cg44-Pg1-Ps210 | Cg44-Pg1-Ps211 | Cg44-Pg1-Ps212 |
| Cg44-Pg1-Ps213 | Cg44-Pg1-Ps214 | Cg44-Pg1-Ps215 | Cg44-Pg1-Ps216 |
| Cg44-Pg1-Ps217 | Cg44-Pg1-Ps218 | Cg44-Pg1-Ps219 | Cg44-Pg1-Ps220 |
| Cg44-Pg1-Ps221 | Cg44-Pg1-Ps222 | Cg44-Pg1-Ps223 | Cg44-Pg1-Ps224 |
| Cg44-Pg1-Ps225 | Cg44-Pg1-Ps226 | Cg44-Pg1-Ps227 | Cg44-Pg1-Ps228 |
| Cg44-Pg1-Ps229 | Cg44-Pg1-Ps230 | Cg44-Pg1-Ps231 | Cg44-Pg1-Ps232 |
| Cg44-Pg1-Ps233 | Cg44-Pg1-Ps234 | Cg44-Pg1-Ps235 | Cg44-Pg1-Ps236 |
| Cg44-Pg1-Ps237 | Cg44-Pg1-Ps238 | Cg44-Pg1-Ps239 | Cg44-Pg1-Ps240 |
| Cg44-Pg1-Ps241 | Cg44-Pg1-Ps242 | Cg44-Pg1-Ps243 | |
| Cg45-Pg1-Ps1 | Cg45-Pg1-Ps2 | Cg45-Pg1-Ps3 | Cg45-Pg1-Ps4 |
| Cg45-Pg1-Ps5 | Cg45-Pg1-Ps6 | Cg45-Pg1-Ps7 | Cg45-Pg1-Ps8 |
| Cg45-Pg1-Ps9 | Cg45-Pg1-Ps10 | Cg45-Pg1-Ps11 | Cg45-Pg1-Ps12 |
| Cg45-Pg1-Ps13 | Cg45-Pg1-Ps14 | Cg45-Pg1-Ps15 | Cg45-Pg1-Ps16 |
| Cg45-Pg1-Ps17 | Cg45-Pg1-Ps18 | Cg45-Pg1-Ps19 | Cg45-Pg1-Ps20 |
| Cg45-Pg1-Ps21 | Cg45-Pg1-Ps22 | Cg45-Pg1-Ps23 | Cg45-Pg1-Ps24 |
| Cg45-Pg1-Ps25 | Cg45-Pg1-Ps26 | Cg45-Pg1-Ps27 | Cg45-Pg1-Ps28 |
| Cg45-Pg1-Ps29 | Cg45-Pg1-Ps30 | Cg45-Pg1-Ps31 | Cg45-Pg1-Ps32 |
| Cg45-Pg1-Ps33 | Cg45-Pg1-Ps34 | Cg45-Pg1-Ps35 | Cg45-Pg1-Ps36 |
| Cg45-Pg1-Ps37 | Cg45-Pg1-Ps38 | Cg45-Pg1-Ps39 | Cg45-Pg1-Ps40 |
| Cg45-Pg1-Ps41 | Cg45-Pg1-Ps42 | Cg45-Pg1-Ps43 | Cg45-Pg1-Ps44 |
| Cg45-Pg1-Ps45 | Cg45-Pg1-Ps46 | Cg45-Pg1-Ps47 | Cg45-Pg1-Ps48 |
| Cg45-Pg1-Ps49 | Cg45-Pg1-Ps50 | Cg45-Pg1-Ps51 | Cg45-Pg1-Ps52 |
| Cg45-Pg1-Ps53 | Cg45-Pg1-Ps54 | Cg45-Pg1-Ps55 | Cg45-Pg1-Ps56 |
| Cg45-Pg1-Ps57 | Cg45-Pg1-Ps58 | Cg45-Pg1-Ps59 | Cg45-Pg1-Ps60 |
| Cg45-Pg1-Ps61 | Cg45-Pg1-Ps62 | Cg45-Pg1-Ps63 | Cg45-Pg1-Ps64 |
| Cg45-Pg1-Ps65 | Cg45-Pg1-Ps66 | Cg45-Pg1-Ps67 | Cg45-Pg1-Ps68 |
| Cg45-Pg1-Ps69 | Cg45-Pg1-Ps70 | Cg45-Pg1-Ps71 | Cg45-Pg1-Ps72 |
| Cg45-Pg1-Ps73 | Cg45-Pg1-Ps74 | Cg45-Pg1-Ps75 | Cg45-Pg1-Ps76 |
| Cg45-Pg1-Ps77 | Cg45-Pg1-Ps78 | Cg45-Pg1-Ps79 | Cg45-Pg1-Ps80 |
| Cg45-Pg1-Ps81 | Cg45-Pg1-Ps82 | Cg45-Pg1-Ps83 | Cg45-Pg1-Ps84 |
| Cg45-Pg1-Ps85 | Cg45-Pg1-Ps86 | Cg45-Pg1-Ps87 | Cg45-Pg1-Ps88 |
| Cg45-Pg1-Ps89 | Cg45-Pg1-Ps90 | Cg45-Pg1-Ps91 | Cg45-Pg1-Ps92 |
| Cg45-Pg1-Ps93 | Cg45-Pg1-Ps94 | Cg45-Pg1-Ps95 | Cg45-Pg1-Ps96 |
| Cg45-Pg1-Ps97 | Cg45-Pg1-Ps98 | Cg45-Pg1-Ps99 | Cg45-Pg1-Ps100 |
| Cg45-Pg1-Ps101 | Cg45-Pg1-Ps102 | Cg45-Pg1-Ps103 | Cg45-Pg1-Ps104 |
| Cg45-Pg1-Ps105 | Cg45-Pg1-Ps106 | Cg45-Pg1-Ps107 | Cg45-Pg1-Ps108 |
| Cg45-Pg1-Ps109 | Cg45-Pg1-Ps110 | Cg45-Pg1-Ps111 | Cg45-Pg1-Ps112 |
| Cg45-Pg1-Ps113 | Cg45-Pg1-Ps114 | Cg45-Pg1-Ps115 | Cg45-Pg1-Ps116 |
| Cg45-Pg1-Ps117 | Cg45-Pg1-Ps118 | Cg45-Pg1-Ps119 | Cg45-Pg1-Ps120 |
| Cg45-Pg1-Ps121 | Cg45-Pg1-Ps122 | Cg45-Pg1-Ps123 | Cg45-Pg1-Ps124 |
| Cg45-Pg1-Ps125 | Cg45-Pg1-Ps126 | Cg45-Pg1-Ps127 | Cg45-Pg1-Ps128 |
| Cg45-Pg1-Ps129 | Cg45-Pg1-Ps130 | Cg45-Pg1-Ps131 | Cg45-Pg1-Ps132 |
| Cg45-Pg1-Ps133 | Cg45-Pg1-Ps134 | Cg45-Pg1-Ps135 | Cg45-Pg1-Ps136 |
| Cg45-Pg1-Ps137 | Cg45-Pg1-Ps138 | Cg45-Pg1-Ps139 | Cg45-Pg1-Ps140 |
| Cg45-Pg1-Ps141 | Cg45-Pg1-Ps142 | Cg45-Pg1-Ps143 | Cg45-Pg1-Ps144 |
| Cg45-Pg1-Ps145 | Cg45-Pg1-Ps146 | Cg45-Pg1-Ps147 | Cg45-Pg1-Ps148 |
| Cg45-Pg1-Ps149 | Cg45-Pg1-Ps150 | Cg45-Pg1-Ps151 | Cg45-Pg1-Ps152 |
| Cg45-Pg1-Ps153 | Cg45-Pg1-Ps154 | Cg45-Pg1-Ps155 | Cg45-Pg1-Ps156 |
| Cg45-Pg1-Ps157 | Cg45-Pg1-Ps158 | Cg45-Pg1-Ps159 | Cg45-Pg1-Ps160 |
| Cg45-Pg1-Ps161 | Cg45-Pg1-Ps162 | Cg45-Pg1-Ps163 | Cg45-Pg1-Ps164 |
| Cg45-Pg1-Ps165 | Cg45-Pg1-Ps166 | Cg45-Pg1-Ps167 | Cg45-Pg1-Ps168 |
| Cg45-Pg1-Ps169 | Cg45-Pg1-Ps170 | Cg45-Pg1-Ps171 | Cg45-Pg1-Ps172 |
| Cg45-Pg1-Ps173 | Cg45-Pg1-Ps174 | Cg45-Pg1-Ps175 | Cg45-Pg1-Ps176 |
| Cg45-Pg1-Ps177 | Cg45-Pg1-Ps178 | Cg45-Pg1-Ps179 | Cg45-Pg1-Ps180 |
| Cg45-Pg1-Ps181 | Cg45-Pg1-Ps182 | Cg45-Pg1-Ps183 | Cg45-Pg1-Ps184 |
| Cg45-Pg1-Ps185 | Cg45-Pg1-Ps186 | Cg45-Pg1-Ps187 | Cg45-Pg1-Ps188 |
| Cg45-Pg1-Ps189 | Cg45-Pg1-Ps190 | Cg45-Pg1-Ps191 | Cg45-Pg1-Ps192 |
| Cg45-Pg1-Ps193 | Cg45-Pg1-Ps194 | Cg45-Pg1-Ps195 | Cg45-Pg1-Ps196 |
| Cg45-Pg1-Ps197 | Cg45-Pg1-Ps198 | Cg45-Pg1-Ps199 | Cg45-Pg1-Ps200 |
| Cg45-Pg1-Ps201 | Cg45-Pg1-Ps202 | Cg45-Pg1-Ps203 | Cg45-Pg1-Ps204 |
| Cg45-Pg1-Ps205 | Cg45-Pg1-Ps206 | Cg45-Pg1-Ps207 | Cg45-Pg1-Ps208 |
| Cg45-Pg1-Ps209 | Cg45-Pg1-Ps210 | Cg45-Pg1-Ps211 | Cg45-Pg1-Ps212 |
| Cg45-Pg1-Ps213 | Cg45-Pg1-Ps214 | Cg45-Pg1-Ps215 | Cg45-Pg1-Ps216 |
| Cg45-Pg1-Ps217 | Cg45-Pg1-Ps218 | Cg45-Pg1-Ps219 | Cg45-Pg1-Ps220 |
| Cg45-Pg1-Ps221 | Cg45-Pg1-Ps222 | Cg45-Pg1-Ps223 | Cg45-Pg1-Ps224 |
| Cg45-Pg1-Ps225 | Cg45-Pg1-Ps226 | Cg45-Pg1-Ps227 | Cg45-Pg1-Ps228 |
| Cg45-Pg1-Ps229 | Cg45-Pg1-Ps230 | Cg45-Pg1-Ps231 | Cg45-Pg1-Ps232 |
| Cg45-Pg1-Ps233 | Cg45-Pg1-Ps234 | Cg45-Pg1-Ps235 | Cg45-Pg1-Ps236 |
| Cg45-Pg1-Ps237 | Cg45-Pg1-Ps238 | Cg45-Pg1-Ps239 | Cg45-Pg1-Ps240 |
| Cg45-Pg1-Ps241 | Cg45-Pg1-Ps242 | Cg45-Pg1-Ps243 | |
| Cg46-Pg1-Ps1 | Cg46-Pg1-Ps2 | Cg46-Pg1-Ps3 | Cg46-Pg1-Ps4 |
| Cg46-Pg1-Ps5 | Cg46-Pg1-Ps6 | Cg46-Pg1-Ps7 | Cg46-Pg1-Ps8 |
| Cg46-Pg1-Ps9 | Cg46-Pg1-Ps10 | Cg46-Pg1-Ps11 | Cg46-Pg1-Ps12 |
| Cg46-Pg1-Ps13 | Cg46-Pg1-Ps14 | Cg46-Pg1-Ps15 | Cg46-Pg1-Ps16 |
| Cg46-Pg1-Ps17 | Cg46-Pg1-Ps18 | Cg46-Pg1-Ps19 | Cg46-Pg1-Ps20 |
| Cg46-Pg1-Ps21 | Cg46-Pg1-Ps22 | Cg46-Pg1-Ps23 | Cg46-Pg1-Ps24 |
| Cg46-Pg1-Ps25 | Cg46-Pg1-Ps26 | Cg46-Pg1-Ps27 | Cg46-Pg1-Ps28 |
| Cg46-Pg1-Ps29 | Cg46-Pg1-Ps30 | Cg46-Pg1-Ps31 | Cg46-Pg1-Ps32 |
| Cg46-Pg1-Ps33 | Cg46-Pg1-Ps34 | Cg46-Pg1-Ps35 | Cg46-Pg1-Ps36 |
| Cg46-Pg1-Ps37 | Cg46-Pg1-Ps38 | Cg46-Pg1-Ps39 | Cg46-Pg1-Ps40 |
| Cg46-Pg1-Ps41 | Cg46-Pg1-Ps42 | Cg46-Pg1-Ps43 | Cg46-Pg1-Ps44 |
| Cg46-Pg1-Ps45 | Cg46-Pg1-Ps46 | Cg46-Pg1-Ps47 | Cg46-Pg1-Ps48 |
| Cg46-Pg1-Ps49 | Cg46-Pg1-Ps50 | Cg46-Pg1-Ps51 | Cg46-Pg1-Ps52 |
| Cg46-Pg1-Ps53 | Cg46-Pg1-Ps54 | Cg46-Pg1-Ps55 | Cg46-Pg1-Ps56 |
| Cg46-Pg1-Ps57 | Cg46-Pg1-Ps58 | Cg46-Pg1-Ps59 | Cg46-Pg1-Ps60 |
| Cg46-Pg1-Ps61 | Cg46-Pg1-Ps62 | Cg46-Pg1-Ps63 | Cg46-Pg1-Ps64 |
| Cg46-Pg1-Ps65 | Cg46-Pg1-Ps66 | Cg46-Pg1-Ps67 | Cg46-Pg1-Ps68 |
| Cg46-Pg1-Ps69 | Cg46-Pg1-Ps70 | Cg46-Pg1-Ps71 | Cg46-Pg1-Ps72 |
| Cg46-Pg1-Ps73 | Cg46-Pg1-Ps74 | Cg46-Pg1-Ps75 | Cg46-Pg1-Ps76 |
| Cg46-Pg1-Ps77 | Cg46-Pg1-Ps78 | Cg46-Pg1-Ps79 | Cg46-Pg1-Ps80 |
| Cg46-Pg1-Ps81 | Cg46-Pg1-Ps82 | Cg46-Pg1-Ps83 | Cg46-Pg1-Ps84 |
| Cg46-Pg1-Ps85 | Cg46-Pg1-Ps86 | Cg46-Pg1-Ps87 | Cg46-Pg1-Ps88 |
| Cg46-Pg1-Ps89 | Cg46-Pg1-Ps90 | Cg46-Pg1-Ps91 | Cg46-Pg1-Ps92 |
| Cg46-Pg1-Ps93 | Cg46-Pg1-Ps94 | Cg46-Pg1-Ps95 | Cg46-Pg1-Ps96 |
| Cg46-Pg1-Ps97 | Cg46-Pg1-Ps98 | Cg46-Pg1-Ps99 | Cg46-Pg1-Ps100 |
| Cg46-Pg1-Ps101 | Cg46-Pg1-Ps102 | Cg46-Pg1-Ps103 | Cg46-Pg1-Ps104 |
| Cg46-Pg1-Ps105 | Cg46-Pg1-Ps106 | Cg46-Pg1-Ps107 | Cg46-Pg1-Ps108 |
| Cg46-Pg1-Ps109 | Cg46-Pg1-Ps110 | Cg46-Pg1-Ps111 | Cg46-Pg1-Ps112 |
| Cg46-Pg1-Ps113 | Cg46-Pg1-Ps114 | Cg46-Pg1-Ps115 | Cg46-Pg1-Ps116 |
| Cg46-Pg1-Ps117 | Cg46-Pg1-Ps118 | Cg46-Pg1-Ps119 | Cg46-Pg1-Ps120 |
| Cg46-Pg1-Ps121 | Cg46-Pg1-Ps122 | Cg46-Pg1-Ps123 | Cg46-Pg1-Ps124 |
| Cg46-Pg1-Ps125 | Cg46-Pg1-Ps126 | Cg46-Pg1-Ps127 | Cg46-Pg1-Ps128 |
| Cg46-Pg1-Ps129 | Cg46-Pg1-Ps130 | Cg46-Pg1-Ps131 | Cg46-Pg1-Ps132 |
| Cg46-Pg1-Ps133 | Cg46-Pg1-Ps134 | Cg46-Pg1-Ps135 | Cg46-Pg1-Ps136 |
| Cg46-Pg1-Ps137 | Cg46-Pg1-Ps138 | Cg46-Pg1-Ps139 | Cg46-Pg1-Ps140 |
| Cg46-Pg1-Ps141 | Cg46-Pg1-Ps142 | Cg46-Pg1-Ps143 | Cg46-Pg1-Ps144 |
| Cg46-Pg1-Ps145 | Cg46-Pg1-Ps146 | Cg46-Pg1-Ps147 | Cg46-Pg1-Ps148 |
| Cg46-Pg1-Ps149 | Cg46-Pg1-Ps150 | Cg46-Pg1-Ps151 | Cg46-Pg1-Ps152 |
| Cg46-Pg1-Ps153 | Cg46-Pg1-Ps154 | Cg46-Pg1-Ps155 | Cg46-Pg1-Ps156 |
| Cg46-Pg1-Ps157 | Cg46-Pg1-Ps158 | Cg46-Pg1-Ps159 | Cg46-Pg1-Ps160 |
| Cg46-Pg1-Ps161 | Cg46-Pg1-Ps162 | Cg46-Pg1-Ps163 | Cg46-Pg1-Ps164 |
| Cg46-Pg1-Ps165 | Cg46-Pg1-Ps166 | Cg46-Pg1-Ps167 | Cg46-Pg1-Ps168 |
| Cg46-Pg1-Ps169 | Cg46-Pg1-Ps170 | Cg46-Pg1-Ps171 | Cg46-Pg1-Ps172 |
| Cg46-Pg1-Ps173 | Cg46-Pg1-Ps174 | Cg46-Pg1-Ps175 | Cg46-Pg1-Ps176 |
| Cg46-Pg1-Ps177 | Cg46-Pg1-Ps178 | Cg46-Pg1-Ps179 | Cg46-Pg1-Ps180 |
| Cg46-Pg1-Ps181 | Cg46-Pg1-Ps182 | Cg46-Pg1-Ps183 | Cg46-Pg1-Ps184 |
| Cg46-Pg1-Ps185 | Cg46-Pg1-Ps186 | Cg46-Pg1-Ps187 | Cg46-Pg1-Ps188 |
| Cg46-Pg1-Ps189 | Cg46-Pg1-Ps190 | Cg46-Pg1-Ps191 | Cg46-Pg1-Ps192 |
| Cg46-Pg1-Ps193 | Cg46-Pg1-Ps194 | Cg46-Pg1-Ps195 | Cg46-Pg1-Ps196 |
| Cg46-Pg1-Ps197 | Cg46-Pg1-Ps198 | Cg46-Pg1-Ps199 | Cg46-Pg1-Ps200 |
| Cg46-Pg1-Ps201 | Cg46-Pg1-Ps202 | Cg46-Pg1-Ps203 | Cg46-Pg1-Ps204 |
| Cg46-Pg1-Ps205 | Cg46-Pg1-Ps206 | Cg46-Pg1-Ps207 | Cg46-Pg1-Ps208 |
| Cg46-Pg1-Ps209 | Cg46-Pg1-Ps210 | Cg46-Pg1-Ps211 | Cg46-Pg1-Ps212 |
| Cg46-Pg1-Ps213 | Cg46-Pg1-Ps214 | Cg46-Pg1-Ps215 | Cg46-Pg1-Ps216 |
| Cg46-Pg1-Ps217 | Cg46-Pg1-Ps218 | Cg46-Pg1-Ps219 | Cg46-Pg1-Ps220 |
| Cg46-Pg1-Ps221 | Cg46-Pg1-Ps222 | Cg46-Pg1-Ps223 | Cg46-Pg1-Ps224 |
| Cg46-Pg1-Ps225 | Cg46-Pg1-Ps226 | Cg46-Pg1-Ps227 | Cg46-Pg1-Ps228 |
| Cg46-Pg1-Ps229 | Cg46-Pg1-Ps230 | Cg46-Pg1-Ps231 | Cg46-Pg1-Ps232 |
| Cg46-Pg1-Ps233 | Cg46-Pg1-Ps234 | Cg46-Pg1-Ps235 | Cg46-Pg1-Ps236 |
| Cg46-Pg1-Ps237 | Cg46-Pg1-Ps238 | Cg46-Pg1-Ps239 | Cg46-Pg1-Ps240 |
| Cg46-Pg1-Ps241 | Cg46-Pg1-Ps242 | Cg46-Pg1-Ps243 | |
| Cg47-Pg1-Ps1 | Cg47-Pg1-Ps2 | Cg47-Pg1-Ps3 | Cg47-Pg1-Ps4 |
| Cg47-Pg1-Ps5 | Cg47-Pg1-Ps6 | Cg47-Pg1-Ps7 | Cg47-Pg1-Ps8 |
| Cg47-Pg1-Ps9 | Cg47-Pg1-Ps10 | Cg47-Pg1-Ps11 | Cg47-Pg1-Ps12 |
| Cg47-Pg1-Ps13 | Cg47-Pg1-Ps14 | Cg47-Pg1-Ps15 | Cg47-Pg1-Ps16 |
| Cg47-Pg1-Ps17 | Cg47-Pg1-Ps18 | Cg47-Pg1-Ps19 | Cg47-Pg1-Ps20 |
| Cg47-Pg1-Ps21 | Cg47-Pg1-Ps22 | Cg47-Pg1-Ps23 | Cg47-Pg1-Ps24 |
| Cg47-Pg1-Ps25 | Cg47-Pg1-Ps26 | Cg47-Pg1-Ps27 | Cg47-Pg1-Ps28 |
| Cg47-Pg1-Ps29 | Cg47-Pg1-Ps30 | Cg47-Pg1-Ps31 | Cg47-Pg1-Ps32 |
| Cg47-Pg1-Ps33 | Cg47-Pg1-Ps34 | Cg47-Pg1-Ps35 | Cg47-Pg1-Ps36 |
| Cg47-Pg1-Ps37 | Cg47-Pg1-Ps38 | Cg47-Pg1-Ps39 | Cg47-Pg1-Ps40 |
| Cg47-Pg1-Ps41 | Cg47-Pg1-Ps42 | Cg47-Pg1-Ps43 | Cg47-Pg1-Ps44 |
| Cg47-Pg1-Ps45 | Cg47-Pg1-Ps46 | Cg47-Pg1-Ps47 | Cg47-Pg1-Ps48 |
| Cg47-Pg1-Ps49 | Cg47-Pg1-Ps50 | Cg47-Pg1-Ps51 | Cg47-Pg1-Ps52 |
| Cg47-Pg1-Ps53 | Cg47-Pg1-Ps54 | Cg47-Pg1-Ps55 | Cg47-Pg1-Ps56 |
| Cg47-Pg1-Ps57 | Cg47-Pg1-Ps58 | Cg47-Pg1-Ps59 | Cg47-Pg1-Ps60 |
| Cg47-Pg1-Ps61 | Cg47-Pg1-Ps62 | Cg47-Pg1-Ps63 | Cg47-Pg1-Ps64 |
| Cg47-Pg1-Ps65 | Cg47-Pg1-Ps66 | Cg47-Pg1-Ps67 | Cg47-Pg1-Ps68 |
| Cg47-Pg1-Ps69 | Cg47-Pg1-Ps70 | Cg47-Pg1-Ps71 | Cg47-Pg1-Ps72 |
| Cg47-Pg1-Ps73 | Cg47-Pg1-Ps74 | Cg47-Pg1-Ps75 | Cg47-Pg1-Ps76 |
| Cg47-Pg1-Ps77 | Cg47-Pg1-Ps78 | Cg47-Pg1-Ps79 | Cg47-Pg1-Ps80 |
| Cg47-Pg1-Ps81 | Cg47-Pg1-Ps82 | Cg47-Pg1-Ps83 | Cg47-Pg1-Ps84 |
| Cg47-Pg1-Ps85 | Cg47-Pg1-Ps86 | Cg47-Pg1-Ps87 | Cg47-Pg1-Ps88 |
| Cg47-Pg1-Ps89 | Cg47-Pg1-Ps90 | Cg47-Pg1-Ps91 | Cg47-Pg1-Ps92 |
| Cg47-Pg1-Ps93 | Cg47-Pg1-Ps94 | Cg47-Pg1-Ps95 | Cg47-Pg1-Ps96 |
| Cg47-Pg1-Ps97 | Cg47-Pg1-Ps98 | Cg47-Pg1-Ps99 | Cg47-Pg1-Ps100 |
| Cg47-Pg1-Ps101 | Cg47-Pg1-Ps102 | Cg47-Pg1-Ps103 | Cg47-Pg1-Ps104 |
| Cg47-Pg1-Ps105 | Cg47-Pg1-Ps106 | Cg47-Pg1-Ps107 | Cg47-Pg1-Ps108 |
| Cg47-Pg1-Ps109 | Cg47-Pg1-Ps110 | Cg47-Pg1-Ps111 | Cg47-Pg1-Ps112 |
| Cg47-Pg1-Ps113 | Cg47-Pg1-Ps114 | Cg47-Pg1-Ps115 | Cg47-Pg1-Ps116 |
| Cg47-Pg1-Ps117 | Cg47-Pg1-Ps118 | Cg47-Pg1-Ps119 | Cg47-Pg1-Ps120 |
| Cg47-Pg1-Ps121 | Cg47-Pg1-Ps122 | Cg47-Pg1-Ps123 | Cg47-Pg1-Ps124 |
| Cg47-Pg1-Ps125 | Cg47-Pg1-Ps126 | Cg47-Pg1-Ps127 | Cg47-Pg1-Ps128 |
| Cg47-Pg1-Ps129 | Cg47-Pg1-Ps130 | Cg47-Pg1-Ps131 | Cg47-Pg1-Ps132 |
| Cg47-Pg1-Ps133 | Cg47-Pg1-Ps134 | Cg47-Pg1-Ps135 | Cg47-Pg1-Ps136 |
| Cg47-Pg1-Ps137 | Cg47-Pg1-Ps138 | Cg47-Pg1-Ps139 | Cg47-Pg1-Ps140 |
| Cg47-Pg1-Ps141 | Cg47-Pg1-Ps142 | Cg47-Pg1-Ps143 | Cg47-Pg1-Ps144 |
| Cg47-Pg1-Ps145 | Cg47-Pg1-Ps146 | Cg47-Pg1-Ps147 | Cg47-Pg1-Ps148 |
| Cg47-Pg1-Ps149 | Cg47-Pg1-Ps150 | Cg47-Pg1-Ps151 | Cg47-Pg1-Ps152 |
| Cg47-Pg1-Ps153 | Cg47-Pg1-Ps154 | Cg47-Pg1-Ps155 | Cg47-Pg1-Ps156 |
| Cg47-Pg1-Ps157 | Cg47-Pg1-Ps158 | Cg47-Pg1-Ps159 | Cg47-Pg1-Ps160 |
| Cg47-Pg1-Ps161 | Cg47-Pg1-Ps162 | Cg47-Pg1-Ps163 | Cg47-Pg1-Ps164 |
| Cg47-Pg1-Ps165 | Cg47-Pg1-Ps166 | Cg47-Pg1-Ps167 | Cg47-Pg1-Ps168 |
| Cg47-Pg1-Ps169 | Cg47-Pg1-Ps170 | Cg47-Pg1-Ps171 | Cg47-Pg1-Ps172 |
| Cg47-Pg1-Ps173 | Cg47-Pg1-Ps174 | Cg47-Pg1-Ps175 | Cg47-Pg1-Ps176 |
| Cg47-Pg1-Ps177 | Cg47-Pg1-Ps178 | Cg47-Pg1-Ps179 | Cg47-Pg1-Ps180 |
| Cg47-Pg1-Ps181 | Cg47-Pg1-Ps182 | Cg47-Pg1-Ps183 | Cg47-Pg1-Ps184 |
| Cg47-Pg1-Ps185 | Cg47-Pg1-Ps186 | Cg47-Pg1-Ps187 | Cg47-Pg1-Ps188 |
| Cg47-Pg1-Ps189 | Cg47-Pg1-Ps190 | Cg47-Pg1-Ps191 | Cg47-Pg1-Ps192 |
| Cg47-Pg1-Ps193 | Cg47-Pg1-Ps194 | Cg47-Pg1-Ps195 | Cg47-Pg1-Ps196 |
| Cg47-Pg1-Ps197 | Cg47-Pg1-Ps198 | Cg47-Pg1-Ps199 | Cg47-Pg1-Ps200 |
| Cg47-Pg1-Ps201 | Cg47-Pg1-Ps202 | Cg47-Pg1-Ps203 | Cg47-Pg1-Ps204 |
| Cg47-Pg1-Ps205 | Cg47-Pg1-Ps206 | Cg47-Pg1-Ps207 | Cg47-Pg1-Ps208 |
| Cg47-Pg1-Ps209 | Cg47-Pg1-Ps210 | Cg47-Pg1-Ps211 | Cg47-Pg1-Ps212 |
| Cg47-Pg1-Ps213 | Cg47-Pg1-Ps214 | Cg47-Pg1-Ps215 | Cg47-Pg1-Ps216 |
| Cg47-Pg1-Ps217 | Cg47-Pg1-Ps218 | Cg47-Pg1-Ps219 | Cg47-Pg1-Ps220 |
| Cg47-Pg1-Ps221 | Cg47-Pg1-Ps222 | Cg47-Pg1-Ps223 | Cg47-Pg1-Ps224 |
| Cg47-Pg1-Ps225 | Cg47-Pg1-Ps226 | Cg47-Pg1-Ps227 | Cg47-Pg1-Ps228 |
| Cg47-Pg1-Ps229 | Cg47-Pg1-Ps230 | Cg47-Pg1-Ps231 | Cg47-Pg1-Ps232 |
| Cg47-Pg1-Ps233 | Cg47-Pg1-Ps234 | Cg47-Pg1-Ps235 | Cg47-Pg1-Ps236 |
| Cg47-Pg1-Ps237 | Cg47-Pg1-Ps238 | Cg47-Pg1-Ps239 | Cg47-Pg1-Ps240 |
| Cg47-Pg1-Ps241 | Cg47-Pg1-Ps242 | Cg47-Pg1-Ps243 | |
| Cg48-Pg1-Ps1 | Cg48-Pg1-Ps2 | Cg48-Pg1-Ps3 | Cg48-Pg1-Ps4 |
| Cg48-Pg1-Ps5 | Cg48-Pg1-Ps6 | Cg48-Pg1-Ps7 | Cg48-Pg1-Ps8 |
| Cg48-Pg1-Ps9 | Cg48-Pg1-Ps10 | Cg48-Pg1-Ps11 | Cg48-Pg1-Ps12 |
| Cg48-Pg1-Ps13 | Cg48-Pg1-Ps14 | Cg48-Pg1-Ps15 | Cg48-Pg1-Ps16 |
| Cg48-Pg1-Ps17 | Cg48-Pg1-Ps18 | Cg48-Pg1-Ps19 | Cg48-Pg1-Ps20 |
| Cg48-Pg1-Ps21 | Cg48-Pg1-Ps22 | Cg48-Pg1-Ps23 | Cg48-Pg1-Ps24 |
| Cg48-Pg1-Ps25 | Cg48-Pg1-Ps26 | Cg48-Pg1-Ps27 | Cg48-Pg1-Ps28 |
| Cg48-Pg1-Ps29 | Cg48-Pg1-Ps30 | Cg48-Pg1-Ps31 | Cg48-Pg1-Ps32 |
| Cg48-Pg1-Ps33 | Cg48-Pg1-Ps34 | Cg48-Pg1-Ps35 | Cg48-Pg1-Ps36 |
| Cg48-Pg1-Ps37 | Cg48-Pg1-Ps38 | Cg48-Pg1-Ps39 | Cg48-Pg1-Ps40 |
| Cg48-Pg1-Ps41 | Cg48-Pg1-Ps42 | Cg48-Pg1-Ps43 | Cg48-Pg1-Ps44 |
| Cg48-Pg1-Ps45 | Cg48-Pg1-Ps46 | Cg48-Pg1-Ps47 | Cg48-Pg1-Ps48 |
| Cg48-Pg1-Ps49 | Cg48-Pg1-Ps50 | Cg48-Pg1-Ps51 | Cg48-Pg1-Ps52 |
| Cg48-Pg1-Ps53 | Cg48-Pg1-Ps54 | Cg48-Pg1-Ps55 | Cg48-Pg1-Ps56 |
| Cg48-Pg1-Ps57 | Cg48-Pg1-Ps58 | Cg48-Pg1-Ps59 | Cg48-Pg1-Ps60 |
| Cg48-Pg1-Ps61 | Cg48-Pg1-Ps62 | Cg48-Pg1-Ps63 | Cg48-Pg1-Ps64 |
| Cg48-Pg1-Ps65 | Cg48-Pg1-Ps66 | Cg48-Pg1-Ps67 | Cg48-Pg1-Ps68 |
| Cg48-Pg1-Ps69 | Cg48-Pg1-Ps70 | Cg48-Pg1-Ps71 | Cg48-Pg1-Ps72 |
| Cg48-Pg1-Ps73 | Cg48-Pg1-Ps74 | Cg48-Pg1-Ps75 | Cg48-Pg1-Ps76 |
| Cg48-Pg1-Ps77 | Cg48-Pg1-Ps78 | Cg48-Pg1-Ps79 | Cg48-Pg1-Ps80 |
| Cg48-Pg1-Ps81 | Cg48-Pg1-Ps82 | Cg48-Pg1-Ps83 | Cg48-Pg1-Ps84 |
| Cg48-Pg1-Ps85 | Cg48-Pg1-Ps86 | Cg48-Pg1-Ps87 | Cg48-Pg1-Ps88 |
| Cg48-Pg1-Ps89 | Cg48-Pg1-Ps90 | Cg48-Pg1-Ps91 | Cg48-Pg1-Ps92 |
| Cg48-Pg1-Ps93 | Cg48-Pg1-Ps94 | Cg48-Pg1-Ps95 | Cg48-Pg1-Ps96 |
| Cg48-Pg1-Ps97 | Cg48-Pg1-Ps98 | Cg48-Pg1-Ps99 | Cg48-Pg1-Ps100 |
| Cg48-Pg1-Ps101 | Cg48-Pg1-Ps102 | Cg48-Pg1-Ps103 | Cg48-Pg1-Ps104 |
| Cg48-Pg1-Ps105 | Cg48-Pg1-Ps106 | Cg48-Pg1-Ps107 | Cg48-Pg1-Ps108 |
| Cg48-Pg1-Ps109 | Cg48-Pg1-Ps110 | Cg48-Pg1-Ps111 | Cg48-Pg1-Ps112 |
| Cg48-Pg1-Ps113 | Cg48-Pg1-Ps114 | Cg48-Pg1-Ps115 | Cg48-Pg1-Ps116 |
| Cg48-Pg1-Ps117 | Cg48-Pg1-Ps118 | Cg48-Pg1-Ps119 | Cg48-Pg1-Ps120 |
| Cg48-Pg1-Ps121 | Cg48-Pg1-Ps122 | Cg48-Pg1-Ps123 | Cg48-Pg1-Ps124 |
| Cg48-Pg1-Ps125 | Cg48-Pg1-Ps126 | Cg48-Pg1-Ps127 | Cg48-Pg1-Ps128 |
| Cg48-Pg1-Ps129 | Cg48-Pg1-Ps130 | Cg48-Pg1-Ps131 | Cg48-Pg1-Ps132 |
| Cg48-Pg1-Ps133 | Cg48-Pg1-Ps134 | Cg48-Pg1-Ps135 | Cg48-Pg1-Ps136 |
| Cg48-Pg1-Ps137 | Cg48-Pg1-Ps138 | Cg48-Pg1-Ps139 | Cg48-Pg1-Ps140 |
| Cg48-Pg1-Ps141 | Cg48-Pg1-Ps142 | Cg48-Pg1-Ps143 | Cg48-Pg1-Ps144 |
| Cg48-Pg1-Ps145 | Cg48-Pg1-Ps146 | Cg48-Pg1-Ps147 | Cg48-Pg1-Ps148 |
| Cg48-Pg1-Ps149 | Cg48-Pg1-Ps150 | Cg48-Pg1-Ps151 | Cg48-Pg1-Ps152 |
| Cg48-Pg1-Ps153 | Cg48-Pg1-Ps154 | Cg48-Pg1-Ps155 | Cg48-Pg1-Ps156 |
| Cg48-Pg1-Ps157 | Cg48-Pg1-Ps158 | Cg48-Pg1-Ps159 | Cg48-Pg1-Ps160 |
| Cg48-Pg1-Ps161 | Cg48-Pg1-Ps162 | Cg48-Pg1-Ps163 | Cg48-Pg1-Ps164 |
| Cg48-Pg1-Ps165 | Cg48-Pg1-Ps166 | Cg48-Pg1-Ps167 | Cg48-Pg1-Ps168 |
| Cg48-Pg1-Ps169 | Cg48-Pg1-Ps170 | Cg48-Pg1-Ps171 | Cg48-Pg1-Ps172 |
| Cg48-Pg1-Ps173 | Cg48-Pg1-Ps174 | Cg48-Pg1-Ps175 | Cg48-Pg1-Ps176 |
| Cg48-Pg1-Ps177 | Cg48-Pg1-Ps178 | Cg48-Pg1-Ps179 | Cg48-Pg1-Ps180 |
| Cg48-Pg1-Ps181 | Cg48-Pg1-Ps182 | Cg48-Pg1-Ps183 | Cg48-Pg1-Ps184 |
| Cg48-Pg1-Ps185 | Cg48-Pg1-Ps186 | Cg48-Pg1-Ps187 | Cg48-Pg1-Ps188 |
| Cg48-Pg1-Ps189 | Cg48-Pg1-Ps190 | Cg48-Pg1-Ps191 | Cg48-Pg1-Ps192 |
| Cg48-Pg1-Ps193 | Cg48-Pg1-Ps194 | Cg48-Pg1-Ps195 | Cg48-Pg1-Ps196 |
| Cg48-Pg1-Ps197 | Cg48-Pg1-Ps198 | Cg48-Pg1-Ps199 | Cg48-Pg1-Ps200 |
| Cg48-Pg1-Ps201 | Cg48-Pg1-Ps202 | Cg48-Pg1-Ps203 | Cg48-Pg1-Ps204 |
| Cg48-Pg1-Ps205 | Cg48-Pg1-Ps206 | Cg48-Pg1-Ps207 | Cg48-Pg1-Ps208 |
| Cg48-Pg1-Ps209 | Cg48-Pg1-Ps210 | Cg48-Pg1-Ps211 | Cg48-Pg1-Ps212 |
| Cg48-Pg1-Ps213 | Cg48-Pg1-Ps214 | Cg48-Pg1-Ps215 | Cg48-Pg1-Ps216 |
| Cg48-Pg1-Ps217 | Cg48-Pg1-Ps218 | Cg48-Pg1-Ps219 | Cg48-Pg1-Ps220 |
| Cg48-Pg1-Ps221 | Cg48-Pg1-Ps222 | Cg48-Pg1-Ps223 | Cg48-Pg1-Ps224 |
| Cg48-Pg1-Ps225 | Cg48-Pg1-Ps226 | Cg48-Pg1-Ps227 | Cg48-Pg1-Ps228 |
| Cg48-Pg1-Ps229 | Cg48-Pg1-Ps230 | Cg48-Pg1-Ps231 | Cg48-Pg1-Ps232 |
| Cg48-Pg1-Ps233 | Cg48-Pg1-Ps234 | Cg48-Pg1-Ps235 | Cg48-Pg1-Ps236 |
| Cg48-Pg1-Ps237 | Cg48-Pg1-Ps238 | Cg48-Pg1-Ps239 | Cg48-Pg1-Ps240 |
| Cg48-Pg1-Ps241 | Cg48-Pg1-Ps242 | Cg48-Pg1-Ps243 | |
| Cg49-Pg1-Ps1 | Cg49-Pg1-Ps2 | Cg49-Pg1-Ps3 | Cg49-Pg1-Ps4 |
| Cg49-Pg1-Ps5 | Cg49-Pg1-Ps6 | Cg49-Pg1-Ps7 | Cg49-Pg1-Ps8 |
| Cg49-Pg1-Ps9 | Cg49-Pg1-Ps10 | Cg49-Pg1-Ps11 | Cg49-Pg1-Ps12 |
| Cg49-Pg1-Ps13 | Cg49-Pg1-Ps14 | Cg49-Pg1-Ps15 | Cg49-Pg1-Ps16 |
| Cg49-Pg1-Ps17 | Cg49-Pg1-Ps18 | Cg49-Pg1-Ps19 | Cg49-Pg1-Ps20 |
| Cg49-Pg1-Ps21 | Cg49-Pg1-Ps22 | Cg49-Pg1-Ps23 | Cg49-Pg1-Ps24 |
| Cg49-Pg1-Ps25 | Cg49-Pg1-Ps26 | Cg49-Pg1-Ps27 | Cg49-Pg1-Ps28 |
| Cg49-Pg1-Ps29 | Cg49-Pg1-Ps30 | Cg49-Pg1-Ps31 | Cg49-Pg1-Ps32 |
| Cg49-Pg1-Ps33 | Cg49-Pg1-Ps34 | Cg49-Pg1-Ps35 | Cg49-Pg1-Ps36 |
| Cg49-Pg1-Ps37 | Cg49-Pg1-Ps38 | Cg49-Pg1-Ps39 | Cg49-Pg1-Ps40 |
| Cg49-Pg1-Ps41 | Cg49-Pg1-Ps42 | Cg49-Pg1-Ps43 | Cg49-Pg1-Ps44 |
| Cg49-Pg1-Ps45 | Cg49-Pg1-Ps46 | Cg49-Pg1-Ps47 | Cg49-Pg1-Ps48 |
| Cg49-Pg1-Ps49 | Cg49-Pg1-Ps50 | Cg49-Pg1-Ps51 | Cg49-Pg1-Ps52 |
| Cg49-Pg1-Ps53 | Cg49-Pg1-Ps54 | Cg49-Pg1-Ps55 | Cg49-Pg1-Ps56 |
| Cg49-Pg1-Ps57 | Cg49-Pg1-Ps58 | Cg49-Pg1-Ps59 | Cg49-Pg1-Ps60 |
| Cg49-Pg1-Ps61 | Cg49-Pg1-Ps62 | Cg49-Pg1-Ps63 | Cg49-Pg1-Ps64 |
| Cg49-Pg1-Ps65 | Cg49-Pg1-Ps66 | Cg49-Pg1-Ps67 | Cg49-Pg1-Ps68 |
| Cg49-Pg1-Ps69 | Cg49-Pg1-Ps70 | Cg49-Pg1-Ps71 | Cg49-Pg1-Ps72 |
| Cg49-Pg1-Ps73 | Cg49-Pg1-Ps74 | Cg49-Pg1-Ps75 | Cg49-Pg1-Ps76 |
| Cg49-Pg1-Ps77 | Cg49-Pg1-Ps78 | Cg49-Pg1-Ps79 | Cg49-Pg1-Ps80 |
| Cg49-Pg1-Ps81 | Cg49-Pg1-Ps82 | Cg49-Pg1-Ps83 | Cg49-Pg1-Ps84 |
| Cg49-Pg1-Ps85 | Cg49-Pg1-Ps86 | Cg49-Pg1-Ps87 | Cg49-Pg1-Ps88 |
| Cg49-Pg1-Ps89 | Cg49-Pg1-Ps90 | Cg49-Pg1-Ps91 | Cg49-Pg1-Ps92 |
| Cg49-Pg1-Ps93 | Cg49-Pg1-Ps94 | Cg49-Pg1-Ps95 | Cg49-Pg1-Ps96 |
| Cg49-Pg1-Ps97 | Cg49-Pg1-Ps98 | Cg49-Pg1-Ps99 | Cg49-Pg1-Ps100 |
| Cg49-Pg1-Ps101 | Cg49-Pg1-Ps102 | Cg49-Pg1-Ps103 | Cg49-Pg1-Ps104 |
| Cg49-Pg1-Ps105 | Cg49-Pg1-Ps106 | Cg49-Pg1-Ps107 | Cg49-Pg1-Ps108 |
| Cg49-Pg1-Ps109 | Cg49-Pg1-Ps110 | Cg49-Pg1-Ps111 | Cg49-Pg1-Ps112 |
| Cg49-Pg1-Ps113 | Cg49-Pg1-Ps114 | Cg49-Pg1-Ps115 | Cg49-Pg1-Ps116 |
| Cg49-Pg1-Ps117 | Cg49-Pg1-Ps118 | Cg49-Pg1-Ps119 | Cg49-Pg1-Ps120 |
| Cg49-Pg1-Ps121 | Cg49-Pg1-Ps122 | Cg49-Pg1-Ps123 | Cg49-Pg1-Ps124 |
| Cg49-Pg1-Ps125 | Cg49-Pg1-Ps126 | Cg49-Pg1-Ps127 | Cg49-Pg1-Ps128 |
| Cg49-Pg1-Ps129 | Cg49-Pg1-Ps130 | Cg49-Pg1-Ps131 | Cg49-Pg1-Ps132 |
| Cg49-Pg1-Ps133 | Cg49-Pg1-Ps134 | Cg49-Pg1-Ps135 | Cg49-Pg1-Ps136 |
| Cg49-Pg1-Ps137 | Cg49-Pg1-Ps138 | Cg49-Pg1-Ps139 | Cg49-Pg1-Ps140 |
| Cg49-Pg1-Ps141 | Cg49-Pg1-Ps142 | Cg49-Pg1-Ps143 | Cg49-Pg1-Ps144 |
| Cg49-Pg1-Ps145 | Cg49-Pg1-Ps146 | Cg49-Pg1-Ps147 | Cg49-Pg1-Ps148 |
| Cg49-Pg1-Ps149 | Cg49-Pg1-Ps150 | Cg49-Pg1-Ps151 | Cg49-Pg1-Ps152 |
| Cg49-Pg1-Ps153 | Cg49-Pg1-Ps154 | Cg49-Pg1-Ps155 | Cg49-Pg1-Ps156 |
| Cg49-Pg1-Ps157 | Cg49-Pg1-Ps158 | Cg49-Pg1-Ps159 | Cg49-Pg1-Ps160 |
| Cg49-Pg1-Ps161 | Cg49-Pg1-Ps162 | Cg49-Pg1-Ps163 | Cg49-Pg1-Ps164 |
| Cg49-Pg1-Ps165 | Cg49-Pg1-Ps166 | Cg49-Pg1-Ps167 | Cg49-Pg1-Ps168 |
| Cg49-Pg1-Ps169 | Cg49-Pg1-Ps170 | Cg49-Pg1-Ps171 | Cg49-Pg1-Ps172 |
| Cg49-Pg1-Ps173 | Cg49-Pg1-Ps174 | Cg49-Pg1-Ps175 | Cg49-Pg1-Ps176 |
| Cg49-Pg1-Ps177 | Cg49-Pg1-Ps178 | Cg49-Pg1-Ps179 | Cg49-Pg1-Ps180 |
| Cg49-Pg1-Ps181 | Cg49-Pg1-Ps182 | Cg49-Pg1-Ps183 | Cg49-Pg1-Ps184 |
| Cg49-Pg1-Ps185 | Cg49-Pg1-Ps186 | Cg49-Pg1-Ps187 | Cg49-Pg1-Ps188 |
| Cg49-Pg1-Ps189 | Cg49-Pg1-Ps190 | Cg49-Pg1-Ps191 | Cg49-Pg1-Ps192 |
| Cg49-Pg1-Ps193 | Cg49-Pg1-Ps194 | Cg49-Pg1-Ps195 | Cg49-Pg1-Ps196 |
| Cg49-Pg1-Ps197 | Cg49-Pg1-Ps198 | Cg49-Pg1-Ps199 | Cg49-Pg1-Ps200 |
| Cg49-Pg1-Ps201 | Cg49-Pg1-Ps202 | Cg49-Pg1-Ps203 | Cg49-Pg1-Ps204 |
| Cg49-Pg1-Ps205 | Cg49-Pg1-Ps206 | Cg49-Pg1-Ps207 | Cg49-Pg1-Ps208 |
| Cg49-Pg1-Ps209 | Cg49-Pg1-Ps210 | Cg49-Pg1-Ps211 | Cg49-Pg1-Ps212 |
| Cg49-Pg1-Ps213 | Cg49-Pg1-Ps214 | Cg49-Pg1-Ps215 | Cg49-Pg1-Ps216 |
| Cg49-Pg1-Ps217 | Cg49-Pg1-Ps218 | Cg49-Pg1-Ps219 | Cg49-Pg1-Ps220 |
| Cg49-Pg1-Ps221 | Cg49-Pg1-Ps222 | Cg49-Pg1-Ps223 | Cg49-Pg1-Ps224 |
| Cg49-Pg1-Ps225 | Cg49-Pg1-Ps226 | Cg49-Pg1-Ps227 | Cg49-Pg1-Ps228 |
| Cg49-Pg1-Ps229 | Cg49-Pg1-Ps230 | Cg49-Pg1-Ps231 | Cg49-Pg1-Ps232 |
| Cg49-Pg1-Ps233 | Cg49-Pg1-Ps234 | Cg49-Pg1-Ps235 | Cg49-Pg1-Ps236 |
| Cg49-Pg1-Ps237 | Cg49-Pg1-Ps238 | Cg49-Pg1-Ps239 | Cg49-Pg1-Ps240 |
| Cg49-Pg1-Ps241 | Cg49-Pg1-Ps242 | Cg49-Pg1-Ps243 | |
| Cg50-Pg1-Ps1 | Cg50-Pg1-Ps2 | Cg50-Pg1-Ps3 | Cg50-Pg1-Ps4 |
| Cg50-Pg1-Ps5 | Cg50-Pg1-Ps6 | Cg50-Pg1-Ps7 | Cg50-Pg1-Ps8 |
| Cg50-Pg1-Ps9 | Cg50-Pg1-Ps10 | Cg50-Pg1-Ps11 | Cg50-Pg1-Ps12 |
| Cg50-Pg1-Ps13 | Cg50-Pg1-Ps14 | Cg50-Pg1-Ps15 | Cg50-Pg1-Ps16 |
| Cg50-Pg1-Ps17 | Cg50-Pg1-Ps18 | Cg50-Pg1-Ps19 | Cg50-Pg1-Ps20 |
| Cg50-Pg1-Ps21 | Cg50-Pg1-Ps22 | Cg50-Pg1-Ps23 | Cg50-Pg1-Ps24 |
| Cg50-Pg1-Ps25 | Cg50-Pg1-Ps26 | Cg50-Pg1-Ps27 | Cg50-Pg1-Ps28 |
| Cg50-Pg1-Ps29 | Cg50-Pg1-Ps30 | Cg50-Pg1-Ps31 | Cg50-Pg1-Ps32 |
| Cg50-Pg1-Ps33 | Cg50-Pg1-Ps34 | Cg50-Pg1-Ps35 | Cg50-Pg1-Ps36 |
| Cg50-Pg1-Ps37 | Cg50-Pg1-Ps38 | Cg50-Pg1-Ps39 | Cg50-Pg1-Ps40 |
| Cg50-Pg1-Ps41 | Cg50-Pg1-Ps42 | Cg50-Pg1-Ps43 | Cg50-Pg1-Ps44 |
| Cg50-Pg1-Ps45 | Cg50-Pg1-Ps46 | Cg50-Pg1-Ps47 | Cg50-Pg1-Ps48 |
| Cg50-Pg1-Ps49 | Cg50-Pg1-Ps50 | Cg50-Pg1-Ps51 | Cg50-Pg1-Ps52 |
| Cg50-Pg1-Ps53 | Cg50-Pg1-Ps54 | Cg50-Pg1-Ps55 | Cg50-Pg1-Ps56 |
| Cg50-Pg1-Ps57 | Cg50-Pg1-Ps58 | Cg50-Pg1-Ps59 | Cg50-Pg1-Ps60 |
| Cg50-Pg1-Ps61 | Cg50-Pg1-Ps62 | Cg50-Pg1-Ps63 | Cg50-Pg1-Ps64 |
| Cg50-Pg1-Ps65 | Cg50-Pg1-Ps66 | Cg50-Pg1-Ps67 | Cg50-Pg1-Ps68 |
| Cg50-Pg1-Ps69 | Cg50-Pg1-Ps70 | Cg50-Pg1-Ps71 | Cg50-Pg1-Ps72 |
| Cg50-Pg1-Ps73 | Cg50-Pg1-Ps74 | Cg50-Pg1-Ps75 | Cg50-Pg1-Ps76 |
| Cg50-Pg1-Ps77 | Cg50-Pg1-Ps78 | Cg50-Pg1-Ps79 | Cg50-Pg1-Ps80 |
| Cg50-Pg1-Ps81 | Cg50-Pg1-Ps82 | Cg50-Pg1-Ps83 | Cg50-Pg1-Ps84 |
| Cg50-Pg1-Ps85 | Cg50-Pg1-Ps86 | Cg50-Pg1-Ps87 | Cg50-Pg1-Ps88 |
| Cg50-Pg1-Ps89 | Cg50-Pg1-Ps90 | Cg50-Pg1-Ps91 | Cg50-Pg1-Ps92 |
| Cg50-Pg1-Ps93 | Cg50-Pg1-Ps94 | Cg50-Pg1-Ps95 | Cg50-Pg1-Ps96 |
| Cg50-Pg1-Ps97 | Cg50-Pg1-Ps98 | Cg50-Pg1-Ps99 | Cg50-Pg1-Ps100 |
| Cg50-Pg1-Ps101 | Cg50-Pg1-Ps102 | Cg50-Pg1-Ps103 | Cg50-Pg1-Ps104 |
| Cg50-Pg1-Ps105 | Cg50-Pg1-Ps106 | Cg50-Pg1-Ps107 | Cg50-Pg1-Ps108 |
| Cg50-Pg1-Ps109 | Cg50-Pg1-Ps110 | Cg50-Pg1-Ps111 | Cg50-Pg1-Ps112 |
| Cg50-Pg1-Ps113 | Cg50-Pg1-Ps114 | Cg50-Pg1-Ps115 | Cg50-Pg1-Ps116 |
| Cg50-Pg1-Ps117 | Cg50-Pg1-Ps118 | Cg50-Pg1-Ps119 | Cg50-Pg1-Ps120 |
| Cg50-Pg1-Ps121 | Cg50-Pg1-Ps122 | Cg50-Pg1-Ps123 | Cg50-Pg1-Ps124 |
| Cg50-Pg1-Ps125 | Cg50-Pg1-Ps126 | Cg50-Pg1-Ps127 | Cg50-Pg1-Ps128 |
| Cg50-Pg1-Ps129 | Cg50-Pg1-Ps130 | Cg50-Pg1-Ps131 | Cg50-Pg1-Ps132 |
| Cg50-Pg1-Ps133 | Cg50-Pg1-Ps134 | Cg50-Pg1-Ps135 | Cg50-Pg1-Ps136 |
| Cg50-Pg1-Ps137 | Cg50-Pg1-Ps138 | Cg50-Pg1-Ps139 | Cg50-Pg1-Ps140 |
| Cg50-Pg1-Ps141 | Cg50-Pg1-Ps142 | Cg50-Pg1-Ps143 | Cg50-Pg1-Ps144 |
| Cg50-Pg1-Ps145 | Cg50-Pg1-Ps146 | Cg50-Pg1-Ps147 | Cg50-Pg1-Ps148 |
| Cg50-Pg1-Ps149 | Cg50-Pg1-Ps150 | Cg50-Pg1-Ps151 | Cg50-Pg1-Ps152 |
| Cg50-Pg1-Ps153 | Cg50-Pg1-Ps154 | Cg50-Pg1-Ps155 | Cg50-Pg1-Ps156 |
| Cg50-Pg1-Ps157 | Cg50-Pg1-Ps158 | Cg50-Pg1-Ps159 | Cg50-Pg1-Ps160 |
| Cg50-Pg1-Ps161 | Cg50-Pg1-Ps162 | Cg50-Pg1-Ps163 | Cg50-Pg1-Ps164 |
| Cg50-Pg1-Ps165 | Cg50-Pg1-Ps166 | Cg50-Pg1-Ps167 | Cg50-Pg1-Ps168 |
| Cg50-Pg1-Ps169 | Cg50-Pg1-Ps170 | Cg50-Pg1-Ps171 | Cg50-Pg1-Ps172 |
| Cg50-Pg1-Ps173 | Cg50-Pg1-Ps174 | Cg50-Pg1-Ps175 | Cg50-Pg1-Ps176 |
| Cg50-Pg1-Ps177 | Cg50-Pg1-Ps178 | Cg50-Pg1-Ps179 | Cg50-Pg1-Ps180 |
| Cg50-Pg1-Ps181 | Cg50-Pg1-Ps182 | Cg50-Pg1-Ps183 | Cg50-Pg1-Ps184 |
| Cg50-Pg1-Ps185 | Cg50-Pg1-Ps186 | Cg50-Pg1-Ps187 | Cg50-Pg1-Ps188 |
| Cg50-Pg1-Ps189 | Cg50-Pg1-Ps190 | Cg50-Pg1-Ps191 | Cg50-Pg1-Ps192 |
| Cg50-Pg1-Ps193 | Cg50-Pg1-Ps194 | Cg50-Pg1-Ps195 | Cg50-Pg1-Ps196 |
| Cg50-Pg1-Ps197 | Cg50-Pg1-Ps198 | Cg50-Pg1-Ps199 | Cg50-Pg1-Ps200 |
| Cg50-Pg1-Ps201 | Cg50-Pg1-Ps202 | Cg50-Pg1-Ps203 | Cg50-Pg1-Ps204 |
| Cg50-Pg1-Ps205 | Cg50-Pg1-Ps206 | Cg50-Pg1-Ps207 | Cg50-Pg1-Ps208 |
| Cg50-Pg1-Ps209 | Cg50-Pg1-Ps210 | Cg50-Pg1-Ps211 | Cg50-Pg1-Ps212 |
| Cg50-Pg1-Ps213 | Cg50-Pg1-Ps214 | Cg50-Pg1-Ps215 | Cg50-Pg1-Ps216 |
| Cg50-Pg1-Ps217 | Cg50-Pg1-Ps218 | Cg50-Pg1-Ps219 | Cg50-Pg1-Ps220 |
| Cg50-Pg1-Ps221 | Cg50-Pg1-Ps222 | Cg50-Pg1-Ps223 | Cg50-Pg1-Ps224 |
| Cg50-Pg1-Ps225 | Cg50-Pg1-Ps226 | Cg50-Pg1-Ps227 | Cg50-Pg1-Ps228 |
| Cg50-Pg1-Ps229 | Cg50-Pg1-Ps230 | Cg50-Pg1-Ps231 | Cg50-Pg1-Ps232 |
| Cg50-Pg1-Ps233 | Cg50-Pg1-Ps234 | Cg50-Pg1-Ps235 | Cg50-Pg1-Ps236 |
| Cg50-Pg1-Ps237 | Cg50-Pg1-Ps238 | Cg50-Pg1-Ps239 | Cg50-Pg1-Ps240 |
| Cg50-Pg1-Ps241 | Cg50-Pg1-Ps242 | Cg50-Pg1-Ps243 | |
| Cg51-Pg1-Ps1 | Cg51-Pg1-Ps2 | Cg51-Pg1-Ps3 | Cg51-Pg1-Ps4 |
| Cg51-Pg1-Ps5 | Cg51-Pg1-Ps6 | Cg51-Pg1-Ps7 | Cg51-Pg1-Ps8 |
| Cg51-Pg1-Ps9 | Cg51-Pg1-Ps10 | Cg51-Pg1-Ps11 | Cg51-Pg1-Ps12 |
| Cg51-Pg1-Ps13 | Cg51-Pg1-Ps14 | Cg51-Pg1-Ps15 | Cg51-Pg1-Ps16 |
| Cg51-Pg1-Ps17 | Cg51-Pg1-Ps18 | Cg51-Pg1-Ps19 | Cg51-Pg1-Ps20 |
| Cg51-Pg1-Ps21 | Cg51-Pg1-Ps22 | Cg51-Pg1-Ps23 | Cg51-Pg1-Ps24 |
| Cg51-Pg1-Ps25 | Cg51-Pg1-Ps26 | Cg51-Pg1-Ps27 | Cg51-Pg1-Ps28 |
| Cg51-Pg1-Ps29 | Cg51-Pg1-Ps30 | Cg51-Pg1-Ps31 | Cg51-Pg1-Ps32 |
| Cg51-Pg1-Ps33 | Cg51-Pg1-Ps34 | Cg51-Pg1-Ps35 | Cg51-Pg1-Ps36 |
| Cg51-Pg1-Ps37 | Cg51-Pg1-Ps38 | Cg51-Pg1-Ps39 | Cg51-Pg1-Ps40 |
| Cg51-Pg1-Ps41 | Cg51-Pg1-Ps42 | Cg51-Pg1-Ps43 | Cg51-Pg1-Ps44 |
| Cg51-Pg1-Ps45 | Cg51-Pg1-Ps46 | Cg51-Pg1-Ps47 | Cg51-Pg1-Ps48 |
| Cg51-Pg1-Ps49 | Cg51-Pg1-Ps50 | Cg51-Pg1-Ps51 | Cg51-Pg1-Ps52 |
| Cg51-Pg1-Ps53 | Cg51-Pg1-Ps54 | Cg51-Pg1-Ps55 | Cg51-Pg1-Ps56 |
| Cg51-Pg1-Ps57 | Cg51-Pg1-Ps58 | Cg51-Pg1-Ps59 | Cg51-Pg1-Ps60 |
| Cg51-Pg1-Ps61 | Cg51-Pg1-Ps62 | Cg51-Pg1-Ps63 | Cg51-Pg1-Ps64 |
| Cg51-Pg1-Ps65 | Cg51-Pg1-Ps66 | Cg51-Pg1-Ps67 | Cg51-Pg1-Ps68 |
| Cg51-Pg1-Ps69 | Cg51-Pg1-Ps70 | Cg51-Pg1-Ps71 | Cg51-Pg1-Ps72 |
| Cg51-Pg1-Ps73 | Cg51-Pg1-Ps74 | Cg51-Pg1-Ps75 | Cg51-Pg1-Ps76 |
| Cg51-Pg1-Ps77 | Cg51-Pg1-Ps78 | Cg51-Pg1-Ps79 | Cg51-Pg1-Ps80 |
| Cg51-Pg1-Ps81 | Cg51-Pg1-Ps82 | Cg51-Pg1-Ps83 | Cg51-Pg1-Ps84 |
| Cg51-Pg1-Ps85 | Cg51-Pg1-Ps86 | Cg51-Pg1-Ps87 | Cg51-Pg1-Ps88 |
| Cg51-Pg1-Ps89 | Cg51-Pg1-Ps90 | Cg51-Pg1-Ps91 | Cg51-Pg1-Ps92 |
| Cg51-Pg1-Ps93 | Cg51-Pg1-Ps94 | Cg51-Pg1-Ps95 | Cg51-Pg1-Ps96 |
| Cg51-Pg1-Ps97 | Cg51-Pg1-Ps98 | Cg51-Pg1-Ps99 | Cg51-Pg1-Ps100 |
| Cg51-Pg1-Ps101 | Cg51-Pg1-Ps102 | Cg51-Pg1-Ps103 | Cg51-Pg1-Ps104 |
| Cg51-Pg1-Ps105 | Cg51-Pg1-Ps106 | Cg51-Pg1-Ps107 | Cg51-Pg1-Ps108 |
| Cg51-Pg1-Ps109 | Cg51-Pg1-Ps110 | Cg51-Pg1-Ps111 | Cg51-Pg1-Ps112 |
| Cg51-Pg1-Ps113 | Cg51-Pg1-Ps114 | Cg51-Pg1-Ps115 | Cg51-Pg1-Ps116 |
| Cg51-Pg1-Ps117 | Cg51-Pg1-Ps118 | Cg51-Pg1-Ps119 | Cg51-Pg1-Ps120 |
| Cg51-Pg1-Ps121 | Cg51-Pg1-Ps122 | Cg51-Pg1-Ps123 | Cg51-Pg1-Ps124 |
| Cg51-Pg1-Ps125 | Cg51-Pg1-Ps126 | Cg51-Pg1-Ps127 | Cg51-Pg1-Ps128 |
| Cg51-Pg1-Ps129 | Cg51-Pg1-Ps130 | Cg51-Pg1-Ps131 | Cg51-Pg1-Ps132 |
| Cg51-Pg1-Ps133 | Cg51-Pg1-Ps134 | Cg51-Pg1-Ps135 | Cg51-Pg1-Ps136 |
| Cg51-Pg1-Ps137 | Cg51-Pg1-Ps138 | Cg51-Pg1-Ps139 | Cg51-Pg1-Ps140 |
| Cg51-Pg1-Ps141 | Cg51-Pg1-Ps142 | Cg51-Pg1-Ps143 | Cg51-Pg1-Ps144 |
| Cg51-Pg1-Ps145 | Cg51-Pg1-Ps146 | Cg51-Pg1-Ps147 | Cg51-Pg1-Ps148 |
| Cg51-Pg1-Ps149 | Cg51-Pg1-Ps150 | Cg51-Pg1-Ps151 | Cg51-Pg1-Ps152 |
| Cg51-Pg1-Ps153 | Cg51-Pg1-Ps154 | Cg51-Pg1-Ps155 | Cg51-Pg1-Ps156 |
| Cg51-Pg1-Ps157 | Cg51-Pg1-Ps158 | Cg51-Pg1-Ps159 | Cg51-Pg1-Ps160 |
| Cg51-Pg1-Ps161 | Cg51-Pg1-Ps162 | Cg51-Pg1-Ps163 | Cg51-Pg1-Ps164 |
| Cg51-Pg1-Ps165 | Cg51-Pg1-Ps166 | Cg51-Pg1-Ps167 | Cg51-Pg1-Ps168 |
| Cg51-Pg1-Ps169 | Cg51-Pg1-Ps170 | Cg51-Pg1-Ps171 | Cg51-Pg1-Ps172 |
| Cg51-Pg1-Ps173 | Cg51-Pg1-Ps174 | Cg51-Pg1-Ps175 | Cg51-Pg1-Ps176 |
| Cg51-Pg1-Ps177 | Cg51-Pg1-Ps178 | Cg51-Pg1-Ps179 | Cg51-Pg1-Ps180 |
| Cg51-Pg1-Ps181 | Cg51-Pg1-Ps182 | Cg51-Pg1-Ps183 | Cg51-Pg1-Ps184 |
| Cg51-Pg1-Ps185 | Cg51-Pg1-Ps186 | Cg51-Pg1-Ps187 | Cg51-Pg1-Ps188 |
| Cg51-Pg1-Ps189 | Cg51-Pg1-Ps190 | Cg51-Pg1-Ps191 | Cg51-Pg1-Ps192 |
| Cg51-Pg1-Ps193 | Cg51-Pg1-Ps194 | Cg51-Pg1-Ps195 | Cg51-Pg1-Ps196 |
| Cg51-Pg1-Ps197 | Cg51-Pg1-Ps198 | Cg51-Pg1-Ps199 | Cg51-Pg1-Ps200 |
| Cg51-Pg1-Ps201 | Cg51-Pg1-Ps202 | Cg51-Pg1-Ps203 | Cg51-Pg1-Ps204 |
| Cg51-Pg1-Ps205 | Cg51-Pg1-Ps206 | Cg51-Pg1-Ps207 | Cg51-Pg1-Ps208 |
| Cg51-Pg1-Ps209 | Cg51-Pg1-Ps210 | Cg51-Pg1-Ps211 | Cg51-Pg1-Ps212 |
| Cg51-Pg1-Ps213 | Cg51-Pg1-Ps214 | Cg51-Pg1-Ps215 | Cg51-Pg1-Ps216 |
| Cg51-Pg1-Ps217 | Cg51-Pg1-Ps218 | Cg51-Pg1-Ps219 | Cg51-Pg1-Ps220 |
| Cg51-Pg1-Ps221 | Cg51-Pg1-Ps222 | Cg51-Pg1-Ps223 | Cg51-Pg1-Ps224 |
| Cg51-Pg1-Ps225 | Cg51-Pg1-Ps226 | Cg51-Pg1-Ps227 | Cg51-Pg1-Ps228 |
| Cg51-Pg1-Ps229 | Cg51-Pg1-Ps230 | Cg51-Pg1-Ps231 | Cg51-Pg1-Ps232 |
| Cg51-Pg1-Ps233 | Cg51-Pg1-Ps234 | Cg51-Pg1-Ps235 | Cg51-Pg1-Ps236 |
| Cg51-Pg1-Ps237 | Cg51-Pg1-Ps238 | Cg51-Pg1-Ps239 | Cg51-Pg1-Ps240 |
| Cg51-Pg1-Ps241 | Cg51-Pg1-Ps242 | Cg51-Pg1-Ps243 | |
| Cg52-Pg1-Ps1 | Cg52-Pg1-Ps2 | Cg52-Pg1-Ps3 | Cg52-Pg1-Ps4 |
| Cg52-Pg1-Ps5 | Cg52-Pg1-Ps6 | Cg52-Pg1-Ps7 | Cg52-Pg1-Ps8 |
| Cg52-Pg1-Ps9 | Cg52-Pg1-Ps10 | Cg52-Pg1-Ps11 | Cg52-Pg1-Ps12 |
| Cg52-Pg1-Ps13 | Cg52-Pg1-Ps14 | Cg52-Pg1-Ps15 | Cg52-Pg1-Ps16 |
| Cg52-Pg1-Ps17 | Cg52-Pg1-Ps18 | Cg52-Pg1-Ps19 | Cg52-Pg1-Ps20 |
| Cg52-Pg1-Ps21 | Cg52-Pg1-Ps22 | Cg52-Pg1-Ps23 | Cg52-Pg1-Ps24 |
| Cg52-Pg1-Ps25 | Cg52-Pg1-Ps26 | Cg52-Pg1-Ps27 | Cg52-Pg1-Ps28 |
| Cg52-Pg1-Ps29 | Cg52-Pg1-Ps30 | Cg52-Pg1-Ps31 | Cg52-Pg1-Ps32 |
| Cg52-Pg1-Ps33 | Cg52-Pg1-Ps34 | Cg52-Pg1-Ps35 | Cg52-Pg1-Ps36 |
| Cg52-Pg1-Ps37 | Cg52-Pg1-Ps38 | Cg52-Pg1-Ps39 | Cg52-Pg1-Ps40 |
| Cg52-Pg1-Ps41 | Cg52-Pg1-Ps42 | Cg52-Pg1-Ps43 | Cg52-Pg1-Ps44 |
| Cg52-Pg1-Ps45 | Cg52-Pg1-Ps46 | Cg52-Pg1-Ps47 | Cg52-Pg1-Ps48 |
| Cg52-Pg1-Ps49 | Cg52-Pg1-Ps50 | Cg52-Pg1-Ps51 | Cg52-Pg1-Ps52 |
| Cg52-Pg1-Ps53 | Cg52-Pg1-Ps54 | Cg52-Pg1-Ps55 | Cg52-Pg1-Ps56 |
| Cg52-Pg1-Ps57 | Cg52-Pg1-Ps58 | Cg52-Pg1-Ps59 | Cg52-Pg1-Ps60 |
| Cg52-Pg1-Ps61 | Cg52-Pg1-Ps62 | Cg52-Pg1-Ps63 | Cg52-Pg1-Ps64 |
| Cg52-Pg1-Ps65 | Cg52-Pg1-Ps66 | Cg52-Pg1-Ps67 | Cg52-Pg1-Ps68 |
| Cg52-Pg1-Ps69 | Cg52-Pg1-Ps70 | Cg52-Pg1-Ps71 | Cg52-Pg1-Ps72 |
| Cg52-Pg1-Ps73 | Cg52-Pg1-Ps74 | Cg52-Pg1-Ps75 | Cg52-Pg1-Ps76 |
| Cg52-Pg1-Ps77 | Cg52-Pg1-Ps78 | Cg52-Pg1-Ps79 | Cg52-Pg1-Ps80 |
| Cg52-Pg1-Ps81 | Cg52-Pg1-Ps82 | Cg52-Pg1-Ps83 | Cg52-Pg1-Ps84 |
| Cg52-Pg1-Ps85 | Cg52-Pg1-Ps86 | Cg52-Pg1-Ps87 | Cg52-Pg1-Ps88 |
| Cg52-Pg1-Ps89 | Cg52-Pg1-Ps90 | Cg52-Pg1-Ps91 | Cg52-Pg1-Ps92 |
| Cg52-Pg1-Ps93 | Cg52-Pg1-Ps94 | Cg52-Pg1-Ps95 | Cg52-Pg1-Ps96 |
| Cg52-Pg1-Ps97 | Cg52-Pg1-Ps98 | Cg52-Pg1-Ps99 | Cg52-Pg1-Ps100 |
| Cg52-Pg1-Ps101 | Cg52-Pg1-Ps102 | Cg52-Pg1-Ps103 | Cg52-Pg1-Ps104 |
| Cg52-Pg1-Ps105 | Cg52-Pg1-Ps106 | Cg52-Pg1-Ps107 | Cg52-Pg1-Ps108 |
| Cg52-Pg1-Ps109 | Cg52-Pg1-Ps110 | Cg52-Pg1-Ps111 | Cg52-Pg1-Ps112 |
| Cg52-Pg1-Ps113 | Cg52-Pg1-Ps114 | Cg52-Pg1-Ps115 | Cg52-Pg1-Ps116 |
| Cg52-Pg1-Ps117 | Cg52-Pg1-Ps118 | Cg52-Pg1-Ps119 | Cg52-Pg1-Ps120 |
| Cg52-Pg1-Ps121 | Cg52-Pg1-Ps122 | Cg52-Pg1-Ps123 | Cg52-Pg1-Ps124 |
| Cg52-Pg1-Ps125 | Cg52-Pg1-Ps126 | Cg52-Pg1-Ps127 | Cg52-Pg1-Ps128 |
| Cg52-Pg1-Ps129 | Cg52-Pg1-Ps130 | Cg52-Pg1-Ps131 | Cg52-Pg1-Ps132 |
| Cg52-Pg1-Ps133 | Cg52-Pg1-Ps134 | Cg52-Pg1-Ps135 | Cg52-Pg1-Ps136 |
| Cg52-Pg1-Ps137 | Cg52-Pg1-Ps138 | Cg52-Pg1-Ps139 | Cg52-Pg1-Ps140 |
| Cg52-Pg1-Ps141 | Cg52-Pg1-Ps142 | Cg52-Pg1-Ps143 | Cg52-Pg1-Ps144 |
| Cg52-Pg1-Ps145 | Cg52-Pg1-Ps146 | Cg52-Pg1-Ps147 | Cg52-Pg1-Ps148 |
| Cg52-Pg1-Ps149 | Cg52-Pg1-Ps150 | Cg52-Pg1-Ps151 | Cg52-Pg1-Ps152 |
| Cg52-Pg1-Ps153 | Cg52-Pg1-Ps154 | Cg52-Pg1-Ps155 | Cg52-Pg1-Ps156 |
| Cg52-Pg1-Ps157 | Cg52-Pg1-Ps158 | Cg52-Pg1-Ps159 | Cg52-Pg1-Ps160 |
| Cg52-Pg1-Ps161 | Cg52-Pg1-Ps162 | Cg52-Pg1-Ps163 | Cg52-Pg1-Ps164 |
| Cg52-Pg1-Ps165 | Cg52-Pg1-Ps166 | Cg52-Pg1-Ps167 | Cg52-Pg1-Ps168 |
| Cg52-Pg1-Ps169 | Cg52-Pg1-Ps170 | Cg52-Pg1-Ps171 | Cg52-Pg1-Ps172 |
| Cg52-Pg1-Ps173 | Cg52-Pg1-Ps174 | Cg52-Pg1-Ps175 | Cg52-Pg1-Ps176 |
| Cg52-Pg1-Ps177 | Cg52-Pg1-Ps178 | Cg52-Pg1-Ps179 | Cg52-Pg1-Ps180 |
| Cg52-Pg1-Ps181 | Cg52-Pg1-Ps182 | Cg52-Pg1-Ps183 | Cg52-Pg1-Ps184 |
| Cg52-Pg1-Ps185 | Cg52-Pg1-Ps186 | Cg52-Pg1-Ps187 | Cg52-Pg1-Ps188 |
| Cg52-Pg1-Ps189 | Cg52-Pg1-Ps190 | Cg52-Pg1-Ps191 | Cg52-Pg1-Ps192 |
| Cg52-Pg1-Ps193 | Cg52-Pg1-Ps194 | Cg52-Pg1-Ps195 | Cg52-Pg1-Ps196 |
| Cg52-Pg1-Ps197 | Cg52-Pg1-Ps198 | Cg52-Pg1-Ps199 | Cg52-Pg1-Ps200 |
| Cg52-Pg1-Ps201 | Cg52-Pg1-Ps202 | Cg52-Pg1-Ps203 | Cg52-Pg1-Ps204 |
| Cg52-Pg1-Ps205 | Cg52-Pg1-Ps206 | Cg52-Pg1-Ps207 | Cg52-Pg1-Ps208 |
| Cg52-Pg1-Ps209 | Cg52-Pg1-Ps210 | Cg52-Pg1-Ps211 | Cg52-Pg1-Ps212 |
| Cg52-Pg1-Ps213 | Cg52-Pg1-Ps214 | Cg52-Pg1-Ps215 | Cg52-Pg1-Ps216 |
| Cg52-Pg1-Ps217 | Cg52-Pg1-Ps218 | Cg52-Pg1-Ps219 | Cg52-Pg1-Ps220 |
| Cg52-Pg1-Ps221 | Cg52-Pg1-Ps222 | Cg52-Pg1-Ps223 | Cg52-Pg1-Ps224 |
| Cg52-Pg1-Ps225 | Cg52-Pg1-Ps226 | Cg52-Pg1-Ps227 | Cg52-Pg1-Ps228 |
| Cg52-Pg1-Ps229 | Cg52-Pg1-Ps230 | Cg52-Pg1-Ps231 | Cg52-Pg1-Ps232 |
| Cg52-Pg1-Ps233 | Cg52-Pg1-Ps234 | Cg52-Pg1-Ps235 | Cg52-Pg1-Ps236 |
| Cg52-Pg1-Ps237 | Cg52-Pg1-Ps238 | Cg52-Pg1-Ps239 | Cg52-Pg1-Ps240 |
| Cg52-Pg1-Ps241 | Cg52-Pg1-Ps242 | Cg52-Pg1-Ps243 | |
| Cg53-Pg1-Ps1 | Cg53-Pg1-Ps2 | Cg53-Pg1-Ps3 | Cg53-Pg1-Ps4 |
| Cg53-Pg1-Ps5 | Cg53-Pg1-Ps6 | Cg53-Pg1-Ps7 | Cg53-Pg1-Ps8 |
| Cg53-Pg1-Ps9 | Cg53-Pg1-Ps10 | Cg53-Pg1-Ps11 | Cg53-Pg1-Ps12 |
| Cg53-Pg1-Ps13 | Cg53-Pg1-Ps14 | Cg53-Pg1-Ps15 | Cg53-Pg1-Ps16 |
| Cg53-Pg1-Ps17 | Cg53-Pg1-Ps18 | Cg53-Pg1-Ps19 | Cg53-Pg1-Ps20 |
| Cg53-Pg1-Ps21 | Cg53-Pg1-Ps22 | Cg53-Pg1-Ps23 | Cg53-Pg1-Ps24 |
| Cg53-Pg1-Ps25 | Cg53-Pg1-Ps26 | Cg53-Pg1-Ps27 | Cg53-Pg1-Ps28 |
| Cg53-Pg1-Ps29 | Cg53-Pg1-Ps30 | Cg53-Pg1-Ps31 | Cg53-Pg1-Ps32 |
| Cg53-Pg1-Ps33 | Cg53-Pg1-Ps34 | Cg53-Pg1-Ps35 | Cg53-Pg1-Ps36 |
| Cg53-Pg1-Ps37 | Cg53-Pg1-Ps38 | Cg53-Pg1-Ps39 | Cg53-Pg1-Ps40 |
| Cg53-Pg1-Ps41 | Cg53-Pg1-Ps42 | Cg53-Pg1-Ps43 | Cg53-Pg1-Ps44 |
| Cg53-Pg1-Ps45 | Cg53-Pg1-Ps46 | Cg53-Pg1-Ps47 | Cg53-Pg1-Ps48 |
| Cg53-Pg1-Ps49 | Cg53-Pg1-Ps50 | Cg53-Pg1-Ps51 | Cg53-Pg1-Ps52 |
| Cg53-Pg1-Ps53 | Cg53-Pg1-Ps54 | Cg53-Pg1-Ps55 | Cg53-Pg1-Ps56 |
| Cg53-Pg1-Ps57 | Cg53-Pg1-Ps58 | Cg53-Pg1-Ps59 | Cg53-Pg1-Ps60 |
| Cg53-Pg1-Ps61 | Cg53-Pg1-Ps62 | Cg53-Pg1-Ps63 | Cg53-Pg1-Ps64 |
| Cg53-Pg1-Ps65 | Cg53-Pg1-Ps66 | Cg53-Pg1-Ps67 | Cg53-Pg1-Ps68 |
| Cg53-Pg1-Ps69 | Cg53-Pg1-Ps70 | Cg53-Pg1-Ps71 | Cg53-Pg1-Ps72 |
| Cg53-Pg1-Ps73 | Cg53-Pg1-Ps74 | Cg53-Pg1-Ps75 | Cg53-Pg1-Ps76 |
| Cg53-Pg1-Ps77 | Cg53-Pg1-Ps78 | Cg53-Pg1-Ps79 | Cg53-Pg1-Ps80 |
| Cg53-Pg1-Ps81 | Cg53-Pg1-Ps82 | Cg53-Pg1-Ps83 | Cg53-Pg1-Ps84 |
| Cg53-Pg1-Ps85 | Cg53-Pg1-Ps86 | Cg53-Pg1-Ps87 | Cg53-Pg1-Ps88 |
| Cg53-Pg1-Ps89 | Cg53-Pg1-Ps90 | Cg53-Pg1-Ps91 | Cg53-Pg1-Ps92 |
| Cg53-Pg1-Ps93 | Cg53-Pg1-Ps94 | Cg53-Pg1-Ps95 | Cg53-Pg1-Ps96 |
| Cg53-Pg1-Ps97 | Cg53-Pg1-Ps98 | Cg53-Pg1-Ps99 | Cg53-Pg1-Ps100 |
| Cg53-Pg1-Ps101 | Cg53-Pg1-Ps102 | Cg53-Pg1-Ps103 | Cg53-Pg1-Ps104 |
| Cg53-Pg1-Ps105 | Cg53-Pg1-Ps106 | Cg53-Pg1-Ps107 | Cg53-Pg1-Ps108 |
| Cg53-Pg1-Ps109 | Cg53-Pg1-Ps110 | Cg53-Pg1-Ps111 | Cg53-Pg1-Ps112 |
| Cg53-Pg1-Ps113 | Cg53-Pg1-Ps114 | Cg53-Pg1-Ps115 | Cg53-Pg1-Ps116 |
| Cg53-Pg1-Ps117 | Cg53-Pg1-Ps118 | Cg53-Pg1-Ps119 | Cg53-Pg1-Ps120 |
| Cg53-Pg1-Ps121 | Cg53-Pg1-Ps122 | Cg53-Pg1-Ps123 | Cg53-Pg1-Ps124 |
| Cg53-Pg1-Ps125 | Cg53-Pg1-Ps126 | Cg53-Pg1-Ps127 | Cg53-Pg1-Ps128 |
| Cg53-Pg1-Ps129 | Cg53-Pg1-Ps130 | Cg53-Pg1-Ps131 | Cg53-Pg1-Ps132 |
| Cg53-Pg1-Ps133 | Cg53-Pg1-Ps134 | Cg53-Pg1-Ps135 | Cg53-Pg1-Ps136 |
| Cg53-Pg1-Ps137 | Cg53-Pg1-Ps138 | Cg53-Pg1-Ps139 | Cg53-Pg1-Ps140 |
| Cg53-Pg1-Ps141 | Cg53-Pg1-Ps142 | Cg53-Pg1-Ps143 | Cg53-Pg1-Ps144 |
| Cg53-Pg1-Ps145 | Cg53-Pg1-Ps146 | Cg53-Pg1-Ps147 | Cg53-Pg1-Ps148 |
| Cg53-Pg1-Ps149 | Cg53-Pg1-Ps150 | Cg53-Pg1-Ps151 | Cg53-Pg1-Ps152 |
| Cg53-Pg1-Ps153 | Cg53-Pg1-Ps154 | Cg53-Pg1-Ps155 | Cg53-Pg1-Ps156 |
| Cg53-Pg1-Ps157 | Cg53-Pg1-Ps158 | Cg53-Pg1-Ps159 | Cg53-Pg1-Ps160 |
| Cg53-Pg1-Ps161 | Cg53-Pg1-Ps162 | Cg53-Pg1-Ps163 | Cg53-Pg1-Ps164 |
| Cg53-Pg1-Ps165 | Cg53-Pg1-Ps166 | Cg53-Pg1-Ps167 | Cg53-Pg1-Ps168 |
| Cg53-Pg1-Ps169 | Cg53-Pg1-Ps170 | Cg53-Pg1-Ps171 | Cg53-Pg1-Ps172 |
| Cg53-Pg1-Ps173 | Cg53-Pg1-Ps174 | Cg53-Pg1-Ps175 | Cg53-Pg1-Ps176 |
| Cg53-Pg1-Ps177 | Cg53-Pg1-Ps178 | Cg53-Pg1-Ps179 | Cg53-Pg1-Ps180 |
| Cg53-Pg1-Ps181 | Cg53-Pg1-Ps182 | Cg53-Pg1-Ps183 | Cg53-Pg1-Ps184 |
| Cg53-Pg1-Ps185 | Cg53-Pg1-Ps186 | Cg53-Pg1-Ps187 | Cg53-Pg1-Ps188 |
| Cg53-Pg1-Ps189 | Cg53-Pg1-Ps190 | Cg53-Pg1-Ps191 | Cg53-Pg1-Ps192 |
| Cg53-Pg1-Ps193 | Cg53-Pg1-Ps194 | Cg53-Pg1-Ps195 | Cg53-Pg1-Ps196 |
| Cg53-Pg1-Ps197 | Cg53-Pg1-Ps198 | Cg53-Pg1-Ps199 | Cg53-Pg1-Ps200 |
| Cg53-Pg1-Ps201 | Cg53-Pg1-Ps202 | Cg53-Pg1-Ps203 | Cg53-Pg1-Ps204 |
| Cg53-Pg1-Ps205 | Cg53-Pg1-Ps206 | Cg53-Pg1-Ps207 | Cg53-Pg1-Ps208 |
| Cg53-Pg1-Ps209 | Cg53-Pg1-Ps210 | Cg53-Pg1-Ps211 | Cg53-Pg1-Ps212 |
| Cg53-Pg1-Ps213 | Cg53-Pg1-Ps214 | Cg53-Pg1-Ps215 | Cg53-Pg1-Ps216 |
| Cg53-Pg1-Ps217 | Cg53-Pg1-Ps218 | Cg53-Pg1-Ps219 | Cg53-Pg1-Ps220 |
| Cg53-Pg1-Ps221 | Cg53-Pg1-Ps222 | Cg53-Pg1-Ps223 | Cg53-Pg1-Ps224 |
| Cg53-Pg1-Ps225 | Cg53-Pg1-Ps226 | Cg53-Pg1-Ps227 | Cg53-Pg1-Ps228 |
| Cg53-Pg1-Ps229 | Cg53-Pg1-Ps230 | Cg53-Pg1-Ps231 | Cg53-Pg1-Ps232 |
| Cg53-Pg1-Ps233 | Cg53-Pg1-Ps234 | Cg53-Pg1-Ps235 | Cg53-Pg1-Ps236 |
| Cg53-Pg1-Ps237 | Cg53-Pg1-Ps238 | Cg53-Pg1-Ps239 | Cg53-Pg1-Ps240 |
| Cg53-Pg1-Ps241 | Cg53-Pg1-Ps242 | Cg53-Pg1-Ps243 | |
| Cg54-Pg1-Ps1 | Cg54-Pg1-Ps2 | Cg54-Pg1-Ps3 | Cg54-Pg1-Ps4 |
| Cg54-Pg1-Ps5 | Cg54-Pg1-Ps6 | Cg54-Pg1-Ps7 | Cg54-Pg1-Ps8 |
| Cg54-Pg1-Ps9 | Cg54-Pg1-Ps10 | Cg54-Pg1-Ps11 | Cg54-Pg1-Ps12 |
| Cg54-Pg1-Ps13 | Cg54-Pg1-Ps14 | Cg54-Pg1-Ps15 | Cg54-Pg1-Ps16 |
| Cg54-Pg1-Ps17 | Cg54-Pg1-Ps18 | Cg54-Pg1-Ps19 | Cg54-Pg1-Ps20 |
| Cg54-Pg1-Ps21 | Cg54-Pg1-Ps22 | Cg54-Pg1-Ps23 | Cg54-Pg1-Ps24 |
| Cg54-Pg1-Ps25 | Cg54-Pg1-Ps26 | Cg54-Pg1-Ps27 | Cg54-Pg1-Ps28 |
| Cg54-Pg1-Ps29 | Cg54-Pg1-Ps30 | Cg54-Pg1-Ps31 | Cg54-Pg1-Ps32 |
| Cg54-Pg1-Ps33 | Cg54-Pg1-Ps34 | Cg54-Pg1-Ps35 | Cg54-Pg1-Ps36 |
| Cg54-Pg1-Ps37 | Cg54-Pg1-Ps38 | Cg54-Pg1-Ps39 | Cg54-Pg1-Ps40 |
| Cg54-Pg1-Ps41 | Cg54-Pg1-Ps42 | Cg54-Pg1-Ps43 | Cg54-Pg1-Ps44 |
| Cg54-Pg1-Ps45 | Cg54-Pg1-Ps46 | Cg54-Pg1-Ps47 | Cg54-Pg1-Ps48 |
| Cg54-Pg1-Ps49 | Cg54-Pg1-Ps50 | Cg54-Pg1-Ps51 | Cg54-Pg1-Ps52 |
| Cg54-Pg1-Ps53 | Cg54-Pg1-Ps54 | Cg54-Pg1-Ps55 | Cg54-Pg1-Ps56 |
| Cg54-Pg1-Ps57 | Cg54-Pg1-Ps58 | Cg54-Pg1-Ps59 | Cg54-Pg1-Ps60 |
| Cg54-Pg1-Ps61 | Cg54-Pg1-Ps62 | Cg54-Pg1-Ps63 | Cg54-Pg1-Ps64 |
| Cg54-Pg1-Ps65 | Cg54-Pg1-Ps66 | Cg54-Pg1-Ps67 | Cg54-Pg1-Ps68 |
| Cg54-Pg1-Ps69 | Cg54-Pg1-Ps70 | Cg54-Pg1-Ps71 | Cg54-Pg1-Ps72 |
| Cg54-Pg1-Ps73 | Cg54-Pg1-Ps74 | Cg54-Pg1-Ps75 | Cg54-Pg1-Ps76 |
| Cg54-Pg1-Ps77 | Cg54-Pg1-Ps78 | Cg54-Pg1-Ps79 | Cg54-Pg1-Ps80 |
| Cg54-Pg1-Ps81 | Cg54-Pg1-Ps82 | Cg54-Pg1-Ps83 | Cg54-Pg1-Ps84 |
| Cg54-Pg1-Ps85 | Cg54-Pg1-Ps86 | Cg54-Pg1-Ps87 | Cg54-Pg1-Ps88 |
| Cg54-Pg1-Ps89 | Cg54-Pg1-Ps90 | Cg54-Pg1-Ps91 | Cg54-Pg1-Ps92 |
| Cg54-Pg1-Ps93 | Cg54-Pg1-Ps94 | Cg54-Pg1-Ps95 | Cg54-Pg1-Ps96 |
| Cg54-Pg1-Ps97 | Cg54-Pg1-Ps98 | Cg54-Pg1-Ps99 | Cg54-Pg1-Ps100 |
| Cg54-Pg1-Ps101 | Cg54-Pg1-Ps102 | Cg54-Pg1-Ps103 | Cg54-Pg1-Ps104 |
| Cg54-Pg1-Ps105 | Cg54-Pg1-Ps106 | Cg54-Pg1-Ps107 | Cg54-Pg1-Ps108 |
| Cg54-Pg1-Ps109 | Cg54-Pg1-Ps110 | Cg54-Pg1-Ps111 | Cg54-Pg1-Ps112 |
| Cg54-Pg1-Ps113 | Cg54-Pg1-Ps114 | Cg54-Pg1-Ps115 | Cg54-Pg1-Ps116 |
| Cg54-Pg1-Ps117 | Cg54-Pg1-Ps118 | Cg54-Pg1-Ps119 | Cg54-Pg1-Ps120 |
| Cg54-Pg1-Ps121 | Cg54-Pg1-Ps122 | Cg54-Pg1-Ps123 | Cg54-Pg1-Ps124 |
| Cg54-Pg1-Ps125 | Cg54-Pg1-Ps126 | Cg54-Pg1-Ps127 | Cg54-Pg1-Ps128 |
| Cg54-Pg1-Ps129 | Cg54-Pg1-Ps130 | Cg54-Pg1-Ps131 | Cg54-Pg1-Ps132 |
| Cg54-Pg1-Ps133 | Cg54-Pg1-Ps134 | Cg54-Pg1-Ps135 | Cg54-Pg1-Ps136 |
| Cg54-Pg1-Ps137 | Cg54-Pg1-Ps138 | Cg54-Pg1-Ps139 | Cg54-Pg1-Ps140 |
| Cg54-Pg1-Ps141 | Cg54-Pg1-Ps142 | Cg54-Pg1-Ps143 | Cg54-Pg1-Ps144 |
| Cg54-Pg1-Ps145 | Cg54-Pg1-Ps146 | Cg54-Pg1-Ps147 | Cg54-Pg1-Ps148 |
| Cg54-Pg1-Ps149 | Cg54-Pg1-Ps150 | Cg54-Pg1-Ps151 | Cg54-Pg1-Ps152 |
| Cg54-Pg1-Ps153 | Cg54-Pg1-Ps154 | Cg54-Pg1-Ps155 | Cg54-Pg1-Ps156 |
| Cg54-Pg1-Ps157 | Cg54-Pg1-Ps158 | Cg54-Pg1-Ps159 | Cg54-Pg1-Ps160 |
| Cg54-Pg1-Ps161 | Cg54-Pg1-Ps162 | Cg54-Pg1-Ps163 | Cg54-Pg1-Ps164 |
| Cg54-Pg1-Ps165 | Cg54-Pg1-Ps166 | Cg54-Pg1-Ps167 | Cg54-Pg1-Ps168 |
| Cg54-Pg1-Ps169 | Cg54-Pg1-Ps170 | Cg54-Pg1-Ps171 | Cg54-Pg1-Ps172 |
| Cg54-Pg1-Ps173 | Cg54-Pg1-Ps174 | Cg54-Pg1-Ps175 | Cg54-Pg1-Ps176 |
| Cg54-Pg1-Ps177 | Cg54-Pg1-Ps178 | Cg54-Pg1-Ps179 | Cg54-Pg1-Ps180 |
| Cg54-Pg1-Ps181 | Cg54-Pg1-Ps182 | Cg54-Pg1-Ps183 | Cg54-Pg1-Ps184 |
| Cg54-Pg1-Ps185 | Cg54-Pg1-Ps186 | Cg54-Pg1-Ps187 | Cg54-Pg1-Ps188 |
| Cg54-Pg1-Ps189 | Cg54-Pg1-Ps190 | Cg54-Pg1-Ps191 | Cg54-Pg1-Ps192 |
| Cg54-Pg1-Ps193 | Cg54-Pg1-Ps194 | Cg54-Pg1-Ps195 | Cg54-Pg1-Ps196 |
| Cg54-Pg1-Ps197 | Cg54-Pg1-Ps198 | Cg54-Pg1-Ps199 | Cg54-Pg1-Ps200 |
| Cg54-Pg1-Ps201 | Cg54-Pg1-Ps202 | Cg54-Pg1-Ps203 | Cg54-Pg1-Ps204 |
| Cg54-Pg1-Ps205 | Cg54-Pg1-Ps206 | Cg54-Pg1-Ps207 | Cg54-Pg1-Ps208 |
| Cg54-Pg1-Ps209 | Cg54-Pg1-Ps210 | Cg54-Pg1-Ps211 | Cg54-Pg1-Ps212 |
| Cg54-Pg1-Ps213 | Cg54-Pg1-Ps214 | Cg54-Pg1-Ps215 | Cg54-Pg1-Ps216 |
| Cg54-Pg1-Ps217 | Cg54-Pg1-Ps218 | Cg54-Pg1-Ps219 | Cg54-Pg1-Ps220 |
| Cg54-Pg1-Ps221 | Cg54-Pg1-Ps222 | Cg54-Pg1-Ps223 | Cg54-Pg1-Ps224 |
| Cg54-Pg1-Ps225 | Cg54-Pg1-Ps226 | Cg54-Pg1-Ps227 | Cg54-Pg1-Ps228 |
| Cg54-Pg1-Ps229 | Cg54-Pg1-Ps230 | Cg54-Pg1-Ps231 | Cg54-Pg1-Ps232 |
| Cg54-Pg1-Ps233 | Cg54-Pg1-Ps234 | Cg54-Pg1-Ps235 | Cg54-Pg1-Ps236 |
| Cg54-Pg1-Ps237 | Cg54-Pg1-Ps238 | Cg54-Pg1-Ps239 | Cg54-Pg1-Ps240 |
| Cg54-Pg1-Ps241 | Cg54-Pg1-Ps242 | Cg54-Pg1-Ps243 | |
| Cg57-Pg1-Ps1 | Cg57-Pg1-Ps2 | Cg57-Pg1-Ps3 | Cg57-Pg1-Ps4 |
| Cg57-Pg1-Ps5 | Cg57-Pg1-Ps6 | Cg57-Pg1-Ps7 | Cg57-Pg1-Ps8 |
| Cg57-Pg1-Ps9 | Cg57-Pg1-Ps10 | Cg57-Pg1-Ps11 | Cg57-Pg1-Ps12 |
| Cg57-Pg1-Ps13 | Cg57-Pg1-Ps14 | Cg57-Pg1-Ps15 | Cg57-Pg1-Ps16 |
| Cg57-Pg1-Ps17 | Cg57-Pg1-Ps18 | Cg57-Pg1-Ps19 | Cg57-Pg1-Ps20 |
| Cg57-Pg1-Ps21 | Cg57-Pg1-Ps22 | Cg57-Pg1-Ps23 | Cg57-Pg1-Ps24 |
| Cg57-Pg1-Ps25 | Cg57-Pg1-Ps26 | Cg57-Pg1-Ps27 | Cg57-Pg1-Ps28 |
| Cg57-Pg1-Ps29 | Cg57-Pg1-Ps30 | Cg57-Pg1-Ps31 | Cg57-Pg1-Ps32 |
| Cg57-Pg1-Ps33 | Cg57-Pg1-Ps34 | Cg57-Pg1-Ps35 | Cg57-Pg1-Ps36 |
| Cg57-Pg1-Ps37 | Cg57-Pg1-Ps38 | Cg57-Pg1-Ps39 | Cg57-Pg1-Ps40 |
| Cg57-Pg1-Ps41 | Cg57-Pg1-Ps42 | Cg57-Pg1-Ps43 | Cg57-Pg1-Ps44 |
| Cg57-Pg1-Ps45 | Cg57-Pg1-Ps46 | Cg57-Pg1-Ps47 | Cg57-Pg1-Ps48 |
| Cg57-Pg1-Ps49 | Cg57-Pg1-Ps50 | Cg57-Pg1-Ps51 | Cg57-Pg1-Ps52 |
| Cg57-Pg1-Ps53 | Cg57-Pg1-Ps54 | Cg57-Pg1-Ps55 | Cg57-Pg1-Ps56 |
| Cg57-Pg1-Ps57 | Cg57-Pg1-Ps58 | Cg57-Pg1-Ps59 | Cg57-Pg1-Ps60 |
| Cg57-Pg1-Ps61 | Cg57-Pg1-Ps62 | Cg57-Pg1-Ps63 | Cg57-Pg1-Ps64 |
| Cg57-Pg1-Ps65 | Cg57-Pg1-Ps66 | Cg57-Pg1-Ps67 | Cg57-Pg1-Ps68 |
| Cg57-Pg1-Ps69 | Cg57-Pg1-Ps70 | Cg57-Pg1-Ps71 | Cg57-Pg1-Ps72 |
| Cg57-Pg1-Ps73 | Cg57-Pg1-Ps74 | Cg57-Pg1-Ps75 | Cg57-Pg1-Ps76 |
| Cg57-Pg1-Ps77 | Cg57-Pg1-Ps78 | Cg57-Pg1-Ps79 | Cg57-Pg1-Ps80 |
| Cg57-Pg1-Ps81 | Cg57-Pg1-Ps82 | Cg57-Pg1-Ps83 | Cg57-Pg1-Ps84 |
| Cg57-Pg1-Ps85 | Cg57-Pg1-Ps86 | Cg57-Pg1-Ps87 | Cg57-Pg1-Ps88 |
| Cg57-Pg1-Ps89 | Cg57-Pg1-Ps90 | Cg57-Pg1-Ps91 | Cg57-Pg1-Ps92 |
| Cg57-Pg1-Ps93 | Cg57-Pg1-Ps94 | Cg57-Pg1-Ps95 | Cg57-Pg1-Ps96 |
| Cg57-Pg1-Ps97 | Cg57-Pg1-Ps98 | Cg57-Pg1-Ps99 | Cg57-Pg1-Ps100 |
| Cg57-Pg1-Ps101 | Cg57-Pg1-Ps102 | Cg57-Pg1-Ps103 | Cg57-Pg1-Ps104 |
| Cg57-Pg1-Ps105 | Cg57-Pg1-Ps106 | Cg57-Pg1-Ps107 | Cg57-Pg1-Ps108 |
| Cg57-Pg1-Ps109 | Cg57-Pg1-Ps110 | Cg57-Pg1-Ps111 | Cg57-Pg1-Ps112 |
| Cg57-Pg1-Ps113 | Cg57-Pg1-Ps114 | Cg57-Pg1-Ps115 | Cg57-Pg1-Ps116 |
| Cg57-Pg1-Ps117 | Cg57-Pg1-Ps118 | Cg57-Pg1-Ps119 | Cg57-Pg1-Ps120 |
| Cg57-Pg1-Ps121 | Cg57-Pg1-Ps122 | Cg57-Pg1-Ps123 | Cg57-Pg1-Ps124 |
| Cg57-Pg1-Ps125 | Cg57-Pg1-Ps126 | Cg57-Pg1-Ps127 | Cg57-Pg1-Ps128 |
| Cg57-Pg1-Ps129 | Cg57-Pg1-Ps130 | Cg57-Pg1-Ps131 | Cg57-Pg1-Ps132 |
| Cg57-Pg1-Ps133 | Cg57-Pg1-Ps134 | Cg57-Pg1-Ps135 | Cg57-Pg1-Ps136 |
| Cg57-Pg1-Ps137 | Cg57-Pg1-Ps138 | Cg57-Pg1-Ps139 | Cg57-Pg1-Ps140 |
| Cg57-Pg1-Ps141 | Cg57-Pg1-Ps142 | Cg57-Pg1-Ps143 | Cg57-Pg1-Ps144 |
| Cg57-Pg1-Ps145 | Cg57-Pg1-Ps146 | Cg57-Pg1-Ps147 | Cg57-Pg1-Ps148 |
| Cg57-Pg1-Ps149 | Cg57-Pg1-Ps150 | Cg57-Pg1-Ps151 | Cg57-Pg1-Ps152 |
| Cg57-Pg1-Ps153 | Cg57-Pg1-Ps154 | Cg57-Pg1-Ps155 | Cg57-Pg1-Ps156 |
| Cg57-Pg1-Ps157 | Cg57-Pg1-Ps158 | Cg57-Pg1-Ps159 | Cg57-Pg1-Ps160 |
| Cg57-Pg1-Ps161 | Cg57-Pg1-Ps162 | Cg57-Pg1-Ps163 | Cg57-Pg1-Ps164 |
| Cg57-Pg1-Ps165 | Cg57-Pg1-Ps166 | Cg57-Pg1-Ps167 | Cg57-Pg1-Ps168 |
| Cg57-Pg1-Ps169 | Cg57-Pg1-Ps170 | Cg57-Pg1-Ps171 | Cg57-Pg1-Ps172 |
| Cg57-Pg1-Ps173 | Cg57-Pg1-Ps174 | Cg57-Pg1-Ps175 | Cg57-Pg1-Ps176 |
| Cg57-Pg1-Ps177 | Cg57-Pg1-Ps178 | Cg57-Pg1-Ps179 | Cg57-Pg1-Ps180 |
| Cg57-Pg1-Ps181 | Cg57-Pg1-Ps182 | Cg57-Pg1-Ps183 | Cg57-Pg1-Ps184 |
| Cg57-Pg1-Ps185 | Cg57-Pg1-Ps186 | Cg57-Pg1-Ps187 | Cg57-Pg1-Ps188 |
| Cg57-Pg1-Ps189 | Cg57-Pg1-Ps190 | Cg57-Pg1-Ps191 | Cg57-Pg1-Ps192 |
| Cg57-Pg1-Ps193 | Cg57-Pg1-Ps194 | Cg57-Pg1-Ps195 | Cg57-Pg1-Ps196 |
| Cg57-Pg1-Ps197 | Cg57-Pg1-Ps198 | Cg57-Pg1-Ps199 | Cg57-Pg1-Ps200 |
| Cg57-Pg1-Ps201 | Cg57-Pg1-Ps202 | Cg57-Pg1-Ps203 | Cg57-Pg1-Ps204 |
| Cg57-Pg1-Ps205 | Cg57-Pg1-Ps206 | Cg57-Pg1-Ps207 | Cg57-Pg1-Ps208 |
| Cg57-Pg1-Ps209 | Cg57-Pg1-Ps210 | Cg57-Pg1-Ps211 | Cg57-Pg1-Ps212 |
| Cg57-Pg1-Ps213 | Cg57-Pg1-Ps214 | Cg57-Pg1-Ps215 | Cg57-Pg1-Ps216 |
| Cg57-Pg1-Ps217 | Cg57-Pg1-Ps218 | Cg57-Pg1-Ps219 | Cg57-Pg1-Ps220 |
| Cg57-Pg1-Ps221 | Cg57-Pg1-Ps222 | Cg57-Pg1-Ps223 | Cg57-Pg1-Ps224 |
| Cg57-Pg1-Ps225 | Cg57-Pg1-Ps226 | Cg57-Pg1-Ps227 | Cg57-Pg1-Ps228 |
| Cg57-Pg1-Ps229 | Cg57-Pg1-Ps230 | Cg57-Pg1-Ps231 | Cg57-Pg1-Ps232 |
| Cg57-Pg1-Ps233 | Cg57-Pg1-Ps234 | Cg57-Pg1-Ps235 | Cg57-Pg1-Ps236 |
| Cg57-Pg1-Ps237 | Cg57-Pg1-Ps238 | Cg57-Pg1-Ps239 | Cg57-Pg1-Ps240 |
| Cg57-Pg1-Ps241 | Cg57-Pg1-Ps242 | Cg57-Pg1-Ps243 | |
| Cg60-Pg1-Ps1 | Cg60-Pg1-Ps2 | Cg60-Pg1-Ps3 | Cg60-Pg1-Ps4 |
| Cg60-Pg1-Ps5 | Cg60-Pg1-Ps6 | Cg60-Pg1-Ps7 | Cg60-Pg1-Ps8 |
| Cg60-Pg1-Ps9 | Cg60-Pg1-Ps10 | Cg60-Pg1-Ps11 | Cg60-Pg1-Ps12 |
| Cg60-Pg1-Ps13 | Cg60-Pg1-Ps14 | Cg60-Pg1-Ps15 | Cg60-Pg1-Ps16 |
| Cg60-Pg1-Ps17 | Cg60-Pg1-Ps18 | Cg60-Pg1-Ps19 | Cg60-Pg1-Ps20 |
| Cg60-Pg1-Ps21 | Cg60-Pg1-Ps22 | Cg60-Pg1-Ps23 | Cg60-Pg1-Ps24 |
| Cg60-Pg1-Ps25 | Cg60-Pg1-Ps26 | Cg60-Pg1-Ps27 | Cg60-Pg1-Ps28 |
| Cg60-Pg1-Ps29 | Cg60-Pg1-Ps30 | Cg60-Pg1-Ps31 | Cg60-Pg1-Ps32 |
| Cg60-Pg1-Ps33 | Cg60-Pg1-Ps34 | Cg60-Pg1-Ps35 | Cg60-Pg1-Ps36 |
| Cg60-Pg1-Ps37 | Cg60-Pg1-Ps38 | Cg60-Pg1-Ps39 | Cg60-Pg1-Ps40 |
| Cg60-Pg1-Ps41 | Cg60-Pg1-Ps42 | Cg60-Pg1-Ps43 | Cg60-Pg1-Ps44 |
| Cg60-Pg1-Ps45 | Cg60-Pg1-Ps46 | Cg60-Pg1-Ps47 | Cg60-Pg1-Ps48 |
| Cg60-Pg1-Ps49 | Cg60-Pg1-Ps50 | Cg60-Pg1-Ps51 | Cg60-Pg1-Ps52 |
| Cg60-Pg1-Ps53 | Cg60-Pg1-Ps54 | Cg60-Pg1-Ps55 | Cg60-Pg1-Ps56 |
| Cg60-Pg1-Ps57 | Cg60-Pg1-Ps58 | Cg60-Pg1-Ps59 | Cg60-Pg1-Ps60 |
| Cg60-Pg1-Ps61 | Cg60-Pg1-Ps62 | Cg60-Pg1-Ps63 | Cg60-Pg1-Ps64 |
| Cg60-Pg1-Ps65 | Cg60-Pg1-Ps66 | Cg60-Pg1-Ps67 | Cg60-Pg1-Ps68 |
| Cg60-Pg1-Ps69 | Cg60-Pg1-Ps70 | Cg60-Pg1-Ps71 | Cg60-Pg1-Ps72 |
| Cg60-Pg1-Ps73 | Cg60-Pg1-Ps74 | Cg60-Pg1-Ps75 | Cg60-Pg1-Ps76 |
| Cg60-Pg1-Ps77 | Cg60-Pg1-Ps78 | Cg60-Pg1-Ps79 | Cg60-Pg1-Ps80 |
| Cg60-Pg1-Ps81 | Cg60-Pg1-Ps82 | Cg60-Pg1-Ps83 | Cg60-Pg1-Ps84 |
| Cg60-Pg1-Ps85 | Cg60-Pg1-Ps86 | Cg60-Pg1-Ps87 | Cg60-Pg1-Ps88 |
| Cg60-Pg1-Ps89 | Cg60-Pg1-Ps90 | Cg60-Pg1-Ps91 | Cg60-Pg1-Ps92 |
| Cg60-Pg1-Ps93 | Cg60-Pg1-Ps94 | Cg60-Pg1-Ps95 | Cg60-Pg1-Ps96 |
| Cg60-Pg1-Ps97 | Cg60-Pg1-Ps98 | Cg60-Pg1-Ps99 | Cg60-Pg1-Ps100 |
| Cg60-Pg1-Ps101 | Cg60-Pg1-Ps102 | Cg60-Pg1-Ps103 | Cg60-Pg1-Ps104 |
| Cg60-Pg1-Ps105 | Cg60-Pg1-Ps106 | Cg60-Pg1-Ps107 | Cg60-Pg1-Ps108 |
| Cg60-Pg1-Ps109 | Cg60-Pg1-Ps110 | Cg60-Pg1-Ps111 | Cg60-Pg1-Ps112 |
| Cg60-Pg1-Ps113 | Cg60-Pg1-Ps114 | Cg60-Pg1-Ps115 | Cg60-Pg1-Ps116 |
| Cg60-Pg1-Ps117 | Cg60-Pg1-Ps118 | Cg60-Pg1-Ps119 | Cg60-Pg1-Ps120 |
| Cg60-Pg1-Ps121 | Cg60-Pg1-Ps122 | Cg60-Pg1-Ps123 | Cg60-Pg1-Ps124 |
| Cg60-Pg1-Ps125 | Cg60-Pg1-Ps126 | Cg60-Pg1-Ps127 | Cg60-Pg1-Ps128 |
| Cg60-Pg1-Ps129 | Cg60-Pg1-Ps130 | Cg60-Pg1-Ps131 | Cg60-Pg1-Ps132 |
| Cg60-Pg1-Ps133 | Cg60-Pg1-Ps134 | Cg60-Pg1-Ps135 | Cg60-Pg1-Ps136 |
| Cg60-Pg1-Ps137 | Cg60-Pg1-Ps138 | Cg60-Pg1-Ps139 | Cg60-Pg1-Ps140 |
| Cg60-Pg1-Ps141 | Cg60-Pg1-Ps142 | Cg60-Pg1-Ps143 | Cg60-Pg1-Ps144 |
| Cg60-Pg1-Ps145 | Cg60-Pg1-Ps146 | Cg60-Pg1-Ps147 | Cg60-Pg1-Ps148 |
| Cg60-Pg1-Ps149 | Cg60-Pg1-Ps150 | Cg60-Pg1-Ps151 | Cg60-Pg1-Ps152 |
| Cg60-Pg1-Ps153 | Cg60-Pg1-Ps154 | Cg60-Pg1-Ps155 | Cg60-Pg1-Ps156 |
| Cg60-Pg1-Ps157 | Cg60-Pg1-Ps158 | Cg60-Pg1-Ps159 | Cg60-Pg1-Ps160 |
| Cg60-Pg1-Ps161 | Cg60-Pg1-Ps162 | Cg60-Pg1-Ps163 | Cg60-Pg1-Ps164 |
| Cg60-Pg1-Ps165 | Cg60-Pg1-Ps166 | Cg60-Pg1-Ps167 | Cg60-Pg1-Ps168 |
| Cg60-Pg1-Ps169 | Cg60-Pg1-Ps170 | Cg60-Pg1-Ps171 | Cg60-Pg1-Ps172 |
| Cg60-Pg1-Ps173 | Cg60-Pg1-Ps174 | Cg60-Pg1-Ps175 | Cg60-Pg1-Ps176 |
| Cg60-Pg1-Ps177 | Cg60-Pg1-Ps178 | Cg60-Pg1-Ps179 | Cg60-Pg1-Ps180 |
| Cg60-Pg1-Ps181 | Cg60-Pg1-Ps182 | Cg60-Pg1-Ps183 | Cg60-Pg1-Ps184 |
| Cg60-Pg1-Ps185 | Cg60-Pg1-Ps186 | Cg60-Pg1-Ps187 | Cg60-Pg1-Ps188 |
| Cg60-Pg1-Ps189 | Cg60-Pg1-Ps190 | Cg60-Pg1-Ps191 | Cg60-Pg1-Ps192 |
| Cg60-Pg1-Ps193 | Cg60-Pg1-Ps194 | Cg60-Pg1-Ps195 | Cg60-Pg1-Ps196 |
| Cg60-Pg1-Ps197 | Cg60-Pg1-Ps198 | Cg60-Pg1-Ps199 | Cg60-Pg1-Ps200 |
| Cg60-Pg1-Ps201 | Cg60-Pg1-Ps202 | Cg60-Pg1-Ps203 | Cg60-Pg1-Ps204 |
| Cg60-Pg1-Ps205 | Cg60-Pg1-Ps206 | Cg60-Pg1-Ps207 | Cg60-Pg1-Ps208 |
| Cg60-Pg1-Ps209 | Cg60-Pg1-Ps210 | Cg60-Pg1-Ps211 | Cg60-Pg1-Ps212 |
| Cg60-Pg1-Ps213 | Cg60-Pg1-Ps214 | Cg60-Pg1-Ps215 | Cg60-Pg1-Ps216 |
| Cg60-Pg1-Ps217 | Cg60-Pg1-Ps218 | Cg60-Pg1-Ps219 | Cg60-Pg1-Ps220 |
| Cg60-Pg1-Ps221 | Cg60-Pg1-Ps222 | Cg60-Pg1-Ps223 | Cg60-Pg1-Ps224 |
| Cg60-Pg1-Ps225 | Cg60-Pg1-Ps226 | Cg60-Pg1-Ps227 | Cg60-Pg1-Ps228 |
| Cg60-Pg1-Ps229 | Cg60-Pg1-Ps230 | Cg60-Pg1-Ps231 | Cg60-Pg1-Ps232 |
| Cg60-Pg1-Ps233 | Cg60-Pg1-Ps234 | Cg60-Pg1-Ps235 | Cg60-Pg1-Ps236 |
| Cg60-Pg1-Ps237 | Cg60-Pg1-Ps238 | Cg60-Pg1-Ps239 | Cg60-Pg1-Ps240 |
| Cg60-Pg1-Ps241 | Cg60-Pg1-Ps242 | Cg60-Pg1-Ps243 | |
| Cg61-Pg1-Ps1 | Cg61-Pg1-Ps2 | Cg61-Pg1-Ps3 | Cg61-Pg1-Ps4 |
| Cg61-Pg1-Ps5 | Cg61-Pg1-Ps6 | Cg61-Pg1-Ps7 | Cg61-Pg1-Ps8 |
| Cg61-Pg1-Ps9 | Cg61-Pg1-Ps10 | Cg61-Pg1-Ps11 | Cg61-Pg1-Ps12 |
| Cg61-Pg1-Ps13 | Cg61-Pg1-Ps14 | Cg61-Pg1-Ps15 | Cg61-Pg1-Ps16 |
| Cg61-Pg1-Ps17 | Cg61-Pg1-Ps18 | Cg61-Pg1-Ps19 | Cg61-Pg1-Ps20 |
| Cg61-Pg1-Ps21 | Cg61-Pg1-Ps22 | Cg61-Pg1-Ps23 | Cg61-Pg1-Ps24 |
| Cg61-Pg1-Ps25 | Cg61-Pg1-Ps26 | Cg61-Pg1-Ps27 | Cg61-Pg1-Ps28 |
| Cg61-Pg1-Ps29 | Cg61-Pg1-Ps30 | Cg61-Pg1-Ps31 | Cg61-Pg1-Ps32 |
| Cg61-Pg1-Ps33 | Cg61-Pg1-Ps34 | Cg61-Pg1-Ps35 | Cg61-Pg1-Ps36 |
| Cg61-Pg1-Ps37 | Cg61-Pg1-Ps38 | Cg61-Pg1-Ps39 | Cg61-Pg1-Ps40 |
| Cg61-Pg1-Ps41 | Cg61-Pg1-Ps42 | Cg61-Pg1-Ps43 | Cg61-Pg1-Ps44 |
| Cg61-Pg1-Ps45 | Cg61-Pg1-Ps46 | Cg61-Pg1-Ps47 | Cg61-Pg1-Ps48 |
| Cg61-Pg1-Ps49 | Cg61-Pg1-Ps50 | Cg61-Pg1-Ps51 | Cg61-Pg1-Ps52 |
| Cg61-Pg1-Ps53 | Cg61-Pg1-Ps54 | Cg61-Pg1-Ps55 | Cg61-Pg1-Ps56 |
| Cg61-Pg1-Ps57 | Cg61-Pg1-Ps58 | Cg61-Pg1-Ps59 | Cg61-Pg1-Ps60 |
| Cg61-Pg1-Ps61 | Cg61-Pg1-Ps62 | Cg61-Pg1-Ps63 | Cg61-Pg1-Ps64 |
| Cg61-Pg1-Ps65 | Cg61-Pg1-Ps66 | Cg61-Pg1-Ps67 | Cg61-Pg1-Ps68 |
| Cg61-Pg1-Ps69 | Cg61-Pg1-Ps70 | Cg61-Pg1-Ps71 | Cg61-Pg1-Ps72 |
| Cg61-Pg1-Ps73 | Cg61-Pg1-Ps74 | Cg61-Pg1-Ps75 | Cg61-Pg1-Ps76 |
| Cg61-Pg1-Ps77 | Cg61-Pg1-Ps78 | Cg61-Pg1-Ps79 | Cg61-Pg1-Ps80 |
| Cg61-Pg1-Ps81 | Cg61-Pg1-Ps82 | Cg61-Pg1-Ps83 | Cg61-Pg1-Ps84 |
| Cg61-Pg1-Ps85 | Cg61-Pg1-Ps86 | Cg61-Pg1-Ps87 | Cg61-Pg1-Ps88 |
| Cg61-Pg1-Ps89 | Cg61-Pg1-Ps90 | Cg61-Pg1-Ps91 | Cg61-Pg1-Ps92 |
| Cg61-Pg1-Ps93 | Cg61-Pg1-Ps94 | Cg61-Pg1-Ps95 | Cg61-Pg1-Ps96 |
| Cg61-Pg1-Ps97 | Cg61-Pg1-Ps98 | Cg61-Pg1-Ps99 | Cg61-Pg1-Ps100 |
| Cg61-Pg1-Ps101 | Cg61-Pg1-Ps102 | Cg61-Pg1-Ps103 | Cg61-Pg1-Ps104 |
| Cg61-Pg1-Ps105 | Cg61-Pg1-Ps106 | Cg61-Pg1-Ps107 | Cg61-Pg1-Ps108 |
| Cg61-Pg1-Ps109 | Cg61-Pg1-Ps110 | Cg61-Pg1-Ps111 | Cg61-Pg1-Ps112 |
| Cg61-Pg1-Ps113 | Cg61-Pg1-Ps114 | Cg61-Pg1-Ps115 | Cg61-Pg1-Ps116 |
| Cg61-Pg1-Ps117 | Cg61-Pg1-Ps118 | Cg61-Pg1-Ps119 | Cg61-Pg1-Ps120 |
| Cg61-Pg1-Ps121 | Cg61-Pg1-Ps122 | Cg61-Pg1-Ps123 | Cg61-Pg1-Ps124 |
| Cg61-Pg1-Ps125 | Cg61-Pg1-Ps126 | Cg61-Pg1-Ps127 | Cg61-Pg1-Ps128 |
| Cg61-Pg1-Ps129 | Cg61-Pg1-Ps130 | Cg61-Pg1-Ps131 | Cg61-Pg1-Ps132 |
| Cg61-Pg1-Ps133 | Cg61-Pg1-Ps134 | Cg61-Pg1-Ps135 | Cg61-Pg1-Ps136 |
| Cg61-Pg1-Ps137 | Cg61-Pg1-Ps138 | Cg61-Pg1-Ps139 | Cg61-Pg1-Ps140 |
| Cg61-Pg1-Ps141 | Cg61-Pg1-Ps142 | Cg61-Pg1-Ps143 | Cg61-Pg1-Ps144 |
| Cg61-Pg1-Ps145 | Cg61-Pg1-Ps146 | Cg61-Pg1-Ps147 | Cg61-Pg1-Ps148 |
| Cg61-Pg1-Ps149 | Cg61-Pg1-Ps150 | Cg61-Pg1-Ps151 | Cg61-Pg1-Ps152 |
| Cg61-Pg1-Ps153 | Cg61-Pg1-Ps154 | Cg61-Pg1-Ps155 | Cg61-Pg1-Ps156 |
| Cg61-Pg1-Ps157 | Cg61-Pg1-Ps158 | Cg61-Pg1-Ps159 | Cg61-Pg1-Ps160 |
| Cg61-Pg1-Ps161 | Cg61-Pg1-Ps162 | Cg61-Pg1-Ps163 | Cg61-Pg1-Ps164 |
| Cg61-Pg1-Ps165 | Cg61-Pg1-Ps166 | Cg61-Pg1-Ps167 | Cg61-Pg1-Ps168 |
| Cg61-Pg1-Ps169 | Cg61-Pg1-Ps170 | Cg61-Pg1-Ps171 | Cg61-Pg1-Ps172 |
| Cg61-Pg1-Ps173 | Cg61-Pg1-Ps174 | Cg61-Pg1-Ps175 | Cg61-Pg1-Ps176 |
| Cg61-Pg1-Ps177 | Cg61-Pg1-Ps178 | Cg61-Pg1-Ps179 | Cg61-Pg1-Ps180 |
| Cg61-Pg1-Ps181 | Cg61-Pg1-Ps182 | Cg61-Pg1-Ps183 | Cg61-Pg1-Ps184 |
| Cg61-Pg1-Ps185 | Cg61-Pg1-Ps186 | Cg61-Pg1-Ps187 | Cg61-Pg1-Ps188 |
| Cg61-Pg1-Ps189 | Cg61-Pg1-Ps190 | Cg61-Pg1-Ps191 | Cg61-Pg1-Ps192 |
| Cg61-Pg1-Ps193 | Cg61-Pg1-Ps194 | Cg61-Pg1-Ps195 | Cg61-Pg1-Ps196 |
| Cg61-Pg1-Ps197 | Cg61-Pg1-Ps198 | Cg61-Pg1-Ps199 | Cg61-Pg1-Ps200 |
| Cg61-Pg1-Ps201 | Cg61-Pg1-Ps202 | Cg61-Pg1-Ps203 | Cg61-Pg1-Ps204 |
| Cg61-Pg1-Ps205 | Cg61-Pg1-Ps206 | Cg61-Pg1-Ps207 | Cg61-Pg1-Ps208 |
| Cg61-Pg1-Ps209 | Cg61-Pg1-Ps210 | Cg61-Pg1-Ps211 | Cg61-Pg1-Ps212 |
| Cg61-Pg1-Ps213 | Cg61-Pg1-Ps214 | Cg61-Pg1-Ps215 | Cg61-Pg1-Ps216 |
| Cg61-Pg1-Ps217 | Cg61-Pg1-Ps218 | Cg61-Pg1-Ps219 | Cg61-Pg1-Ps220 |
| Cg61-Pg1-Ps221 | Cg61-Pg1-Ps222 | Cg61-Pg1-Ps223 | Cg61-Pg1-Ps224 |
| Cg61-Pg1-Ps225 | Cg61-Pg1-Ps226 | Cg61-Pg1-Ps227 | Cg61-Pg1-Ps228 |
| Cg61-Pg1-Ps229 | Cg61-Pg1-Ps230 | Cg61-Pg1-Ps231 | Cg61-Pg1-Ps232 |
| Cg61-Pg1-Ps233 | Cg61-Pg1-Ps234 | Cg61-Pg1-Ps235 | Cg61-Pg1-Ps236 |
| Cg61-Pg1-Ps237 | Cg61-Pg1-Ps238 | Cg61-Pg1-Ps239 | Cg61-Pg1-Ps240 |
| Cg61-Pg1-Ps241 | Cg61-Pg1-Ps242 | Cg61-Pg1-Ps243 | |
| Cg62-Pg1-Ps1 | Cg62-Pg1-Ps2 | Cg62-Pg1-Ps3 | Cg62-Pg1-Ps4 |
| Cg62-Pg1-Ps5 | Cg62-Pg1-Ps6 | Cg62-Pg1-Ps7 | Cg62-Pg1-Ps8 |
| Cg62-Pg1-Ps9 | Cg62-Pg1-Ps10 | Cg62-Pg1-Ps11 | Cg62-Pg1-Ps12 |
| Cg62-Pg1-Ps13 | Cg62-Pg1-Ps14 | Cg62-Pg1-Ps15 | Cg62-Pg1-Ps16 |
| Cg62-Pg1-Ps17 | Cg62-Pg1-Ps18 | Cg62-Pg1-Ps19 | Cg62-Pg1-Ps20 |
| Cg62-Pg1-Ps21 | Cg62-Pg1-Ps22 | Cg62-Pg1-Ps23 | Cg62-Pg1-Ps24 |
| Cg62-Pg1-Ps25 | Cg62-Pg1-Ps26 | Cg62-Pg1-Ps27 | Cg62-Pg1-Ps28 |
| Cg62-Pg1-Ps29 | Cg62-Pg1-Ps30 | Cg62-Pg1-Ps31 | Cg62-Pg1-Ps32 |
| Cg62-Pg1-Ps33 | Cg62-Pg1-Ps34 | Cg62-Pg1-Ps35 | Cg62-Pg1-Ps36 |
| Cg62-Pg1-Ps37 | Cg62-Pg1-Ps38 | Cg62-Pg1-Ps39 | Cg62-Pg1-Ps40 |
| Cg62-Pg1-Ps41 | Cg62-Pg1-Ps42 | Cg62-Pg1-Ps43 | Cg62-Pg1-Ps44 |
| Cg62-Pg1-Ps45 | Cg62-Pg1-Ps46 | Cg62-Pg1-Ps47 | Cg62-Pg1-Ps48 |
| Cg62-Pg1-Ps49 | Cg62-Pg1-Ps50 | Cg62-Pg1-Ps51 | Cg62-Pg1-Ps52 |
| Cg62-Pg1-Ps53 | Cg62-Pg1-Ps54 | Cg62-Pg1-Ps55 | Cg62-Pg1-Ps56 |
| Cg62-Pg1-Ps57 | Cg62-Pg1-Ps58 | Cg62-Pg1-Ps59 | Cg62-Pg1-Ps60 |
| Cg62-Pg1-Ps61 | Cg62-Pg1-Ps62 | Cg62-Pg1-Ps63 | Cg62-Pg1-Ps64 |
| Cg62-Pg1-Ps65 | Cg62-Pg1-Ps66 | Cg62-Pg1-Ps67 | Cg62-Pg1-Ps68 |
| Cg62-Pg1-Ps69 | Cg62-Pg1-Ps70 | Cg62-Pg1-Ps71 | Cg62-Pg1-Ps72 |
| Cg62-Pg1-Ps73 | Cg62-Pg1-Ps74 | Cg62-Pg1-Ps75 | Cg62-Pg1-Ps76 |
| Cg62-Pg1-Ps77 | Cg62-Pg1-Ps78 | Cg62-Pg1-Ps79 | Cg62-Pg1-Ps80 |
| Cg62-Pg1-Ps81 | Cg62-Pg1-Ps82 | Cg62-Pg1-Ps83 | Cg62-Pg1-Ps84 |
| Cg62-Pg1-Ps85 | Cg62-Pg1-Ps86 | Cg62-Pg1-Ps87 | Cg62-Pg1-Ps88 |
| Cg62-Pg1-Ps89 | Cg62-Pg1-Ps90 | Cg62-Pg1-Ps91 | Cg62-Pg1-Ps92 |
| Cg62-Pg1-Ps93 | Cg62-Pg1-Ps94 | Cg62-Pg1-Ps95 | Cg62-Pg1-Ps96 |
| Cg62-Pg1-Ps97 | Cg62-Pg1-Ps98 | Cg62-Pg1-Ps99 | Cg62-Pg1-Ps100 |
| Cg62-Pg1-Ps101 | Cg62-Pg1-Ps102 | Cg62-Pg1-Ps103 | Cg62-Pg1-Ps104 |
| Cg62-Pg1-Ps105 | Cg62-Pg1-Ps106 | Cg62-Pg1-Ps107 | Cg62-Pg1-Ps108 |
| Cg62-Pg1-Ps109 | Cg62-Pg1-Ps110 | Cg62-Pg1-Ps111 | Cg62-Pg1-Ps112 |
| Cg62-Pg1-Ps113 | Cg62-Pg1-Ps114 | Cg62-Pg1-Ps115 | Cg62-Pg1-Ps116 |
| Cg62-Pg1-Ps117 | Cg62-Pg1-Ps118 | Cg62-Pg1-Ps119 | Cg62-Pg1-Ps120 |
| Cg62-Pg1-Ps121 | Cg62-Pg1-Ps122 | Cg62-Pg1-Ps123 | Cg62-Pg1-Ps124 |
| Cg62-Pg1-Ps125 | Cg62-Pg1-Ps126 | Cg62-Pg1-Ps127 | Cg62-Pg1-Ps128 |
| Cg62-Pg1-Ps129 | Cg62-Pg1-Ps130 | Cg62-Pg1-Ps131 | Cg62-Pg1-Ps132 |
| Cg62-Pg1-Ps133 | Cg62-Pg1-Ps134 | Cg62-Pg1-Ps135 | Cg62-Pg1-Ps136 |
| Cg62-Pg1-Ps137 | Cg62-Pg1-Ps138 | Cg62-Pg1-Ps139 | Cg62-Pg1-Ps140 |
| Cg62-Pg1-Ps141 | Cg62-Pg1-Ps142 | Cg62-Pg1-Ps143 | Cg62-Pg1-Ps144 |
| Cg62-Pg1-Ps145 | Cg62-Pg1-Ps146 | Cg62-Pg1-Ps147 | Cg62-Pg1-Ps148 |
| Cg62-Pg1-Ps149 | Cg62-Pg1-Ps150 | Cg62-Pg1-Ps151 | Cg62-Pg1-Ps152 |
| Cg62-Pg1-Ps153 | Cg62-Pg1-Ps154 | Cg62-Pg1-Ps155 | Cg62-Pg1-Ps156 |
| Cg62-Pg1-Ps157 | Cg62-Pg1-Ps158 | Cg62-Pg1-Ps159 | Cg62-Pg1-Ps160 |
| Cg62-Pg1-Ps161 | Cg62-Pg1-Ps162 | Cg62-Pg1-Ps163 | Cg62-Pg1-Ps164 |
| Cg62-Pg1-Ps165 | Cg62-Pg1-Ps166 | Cg62-Pg1-Ps167 | Cg62-Pg1-Ps168 |
| Cg62-Pg1-Ps169 | Cg62-Pg1-Ps170 | Cg62-Pg1-Ps171 | Cg62-Pg1-Ps172 |
| Cg62-Pg1-Ps173 | Cg62-Pg1-Ps174 | Cg62-Pg1-Ps175 | Cg62-Pg1-Ps176 |
| Cg62-Pg1-Ps177 | Cg62-Pg1-Ps178 | Cg62-Pg1-Ps179 | Cg62-Pg1-Ps180 |
| Cg62-Pg1-Ps181 | Cg62-Pg1-Ps182 | Cg62-Pg1-Ps183 | Cg62-Pg1-Ps184 |
| Cg62-Pg1-Ps185 | Cg62-Pg1-Ps186 | Cg62-Pg1-Ps187 | Cg62-Pg1-Ps188 |
| Cg62-Pg1-Ps189 | Cg62-Pg1-Ps190 | Cg62-Pg1-Ps191 | Cg62-Pg1-Ps192 |
| Cg62-Pg1-Ps193 | Cg62-Pg1-Ps194 | Cg62-Pg1-Ps195 | Cg62-Pg1-Ps196 |
| Cg62-Pg1-Ps197 | Cg62-Pg1-Ps198 | Cg62-Pg1-Ps199 | Cg62-Pg1-Ps200 |
| Cg62-Pg1-Ps201 | Cg62-Pg1-Ps202 | Cg62-Pg1-Ps203 | Cg62-Pg1-Ps204 |
| Cg62-Pg1-Ps205 | Cg62-Pg1-Ps206 | Cg62-Pg1-Ps207 | Cg62-Pg1-Ps208 |
| Cg62-Pg1-Ps209 | Cg62-Pg1-Ps210 | Cg62-Pg1-Ps211 | Cg62-Pg1-Ps212 |
| Cg62-Pg1-Ps213 | Cg62-Pg1-Ps214 | Cg62-Pg1-Ps215 | Cg62-Pg1-Ps216 |
| Cg62-Pg1-Ps217 | Cg62-Pg1-Ps218 | Cg62-Pg1-Ps219 | Cg62-Pg1-Ps220 |
| Cg62-Pg1-Ps221 | Cg62-Pg1-Ps222 | Cg62-Pg1-Ps223 | Cg62-Pg1-Ps224 |
| Cg62-Pg1-Ps225 | Cg62-Pg1-Ps226 | Cg62-Pg1-Ps227 | Cg62-Pg1-Ps228 |
| Cg62-Pg1-Ps229 | Cg62-Pg1-Ps230 | Cg62-Pg1-Ps231 | Cg62-Pg1-Ps232 |
| Cg62-Pg1-Ps233 | Cg62-Pg1-Ps234 | Cg62-Pg1-Ps235 | Cg62-Pg1-Ps236 |
| Cg62-Pg1-Ps237 | Cg62-Pg1-Ps238 | Cg62-Pg1-Ps239 | Cg62-Pg1-Ps240 |
| Cg62-Pg1-Ps241 | Cg62-Pg1-Ps242 | Cg62-Pg1-Ps243 | |
| Cg63-Pg1-Ps1 | Cg63-Pg1-Ps2 | Cg63-Pg1-Ps3 | Cg63-Pg1-Ps4 |
| Cg63-Pg1-Ps5 | Cg63-Pg1-Ps6 | Cg63-Pg1-Ps7 | Cg63-Pg1-Ps8 |
| Cg63-Pg1-Ps9 | Cg63-Pg1-Ps10 | Cg63-Pg1-Ps11 | Cg63-Pg1-Ps12 |
| Cg63-Pg1-Ps13 | Cg63-Pg1-Ps14 | Cg63-Pg1-Ps15 | Cg63-Pg1-Ps16 |
| Cg63-Pg1-Ps17 | Cg63-Pg1-Ps18 | Cg63-Pg1-Ps19 | Cg63-Pg1-Ps20 |
| Cg63-Pg1-Ps21 | Cg63-Pg1-Ps22 | Cg63-Pg1-Ps23 | Cg63-Pg1-Ps24 |
| Cg63-Pg1-Ps25 | Cg63-Pg1-Ps26 | Cg63-Pg1-Ps27 | Cg63-Pg1-Ps28 |
| Cg63-Pg1-Ps29 | Cg63-Pg1-Ps30 | Cg63-Pg1-Ps31 | Cg63-Pg1-Ps32 |
| Cg63-Pg1-Ps33 | Cg63-Pg1-Ps34 | Cg63-Pg1-Ps35 | Cg63-Pg1-Ps36 |
| Cg63-Pg1-Ps37 | Cg63-Pg1-Ps38 | Cg63-Pg1-Ps39 | Cg63-Pg1-Ps40 |
| Cg63-Pg1-Ps41 | Cg63-Pg1-Ps42 | Cg63-Pg1-Ps43 | Cg63-Pg1-Ps44 |
| Cg63-Pg1-Ps45 | Cg63-Pg1-Ps46 | Cg63-Pg1-Ps47 | Cg63-Pg1-Ps48 |
| Cg63-Pg1-Ps49 | Cg63-Pg1-Ps50 | Cg63-Pg1-Ps51 | Cg63-Pg1-Ps52 |
| Cg63-Pg1-Ps53 | Cg63-Pg1-Ps54 | Cg63-Pg1-Ps55 | Cg63-Pg1-Ps56 |
| Cg63-Pg1-Ps57 | Cg63-Pg1-Ps58 | Cg63-Pg1-Ps59 | Cg63-Pg1-Ps60 |
| Cg63-Pg1-Ps61 | Cg63-Pg1-Ps62 | Cg63-Pg1-Ps63 | Cg63-Pg1-Ps64 |
| Cg63-Pg1-Ps65 | Cg63-Pg1-Ps66 | Cg63-Pg1-Ps67 | Cg63-Pg1-Ps68 |
| Cg63-Pg1-Ps69 | Cg63-Pg1-Ps70 | Cg63-Pg1-Ps71 | Cg63-Pg1-Ps72 |
| Cg63-Pg1-Ps73 | Cg63-Pg1-Ps74 | Cg63-Pg1-Ps75 | Cg63-Pg1-Ps76 |
| Cg63-Pg1-Ps77 | Cg63-Pg1-Ps78 | Cg63-Pg1-Ps79 | Cg63-Pg1-Ps80 |
| Cg63-Pg1-Ps81 | Cg63-Pg1-Ps82 | Cg63-Pg1-Ps83 | Cg63-Pg1-Ps84 |
| Cg63-Pg1-Ps85 | Cg63-Pg1-Ps86 | Cg63-Pg1-Ps87 | Cg63-Pg1-Ps88 |
| Cg63-Pg1-Ps89 | Cg63-Pg1-Ps90 | Cg63-Pg1-Ps91 | Cg63-Pg1-Ps92 |
| Cg63-Pg1-Ps93 | Cg63-Pg1-Ps94 | Cg63-Pg1-Ps95 | Cg63-Pg1-Ps96 |
| Cg63-Pg1-Ps97 | Cg63-Pg1-Ps98 | Cg63-Pg1-Ps99 | Cg63-Pg1-Ps100 |
| Cg63-Pg1-Ps101 | Cg63-Pg1-Ps102 | Cg63-Pg1-Ps103 | Cg63-Pg1-Ps104 |
| Cg63-Pg1-Ps105 | Cg63-Pg1-Ps106 | Cg63-Pg1-Ps107 | Cg63-Pg1-Ps108 |
| Cg63-Pg1-Ps109 | Cg63-Pg1-Ps110 | Cg63-Pg1-Ps111 | Cg63-Pg1-Ps112 |
| Cg63-Pg1-Ps113 | Cg63-Pg1-Ps114 | Cg63-Pg1-Ps115 | Cg63-Pg1-Ps116 |
| Cg63-Pg1-Ps117 | Cg63-Pg1-Ps118 | Cg63-Pg1-Ps119 | Cg63-Pg1-Ps120 |
| Cg63-Pg1-Ps121 | Cg63-Pg1-Ps122 | Cg63-Pg1-Ps123 | Cg63-Pg1-Ps124 |
| Cg63-Pg1-Ps125 | Cg63-Pg1-Ps126 | Cg63-Pg1-Ps127 | Cg63-Pg1-Ps128 |
| Cg63-Pg1-Ps129 | Cg63-Pg1-Ps130 | Cg63-Pg1-Ps131 | Cg63-Pg1-Ps132 |
| Cg63-Pg1-Ps133 | Cg63-Pg1-Ps134 | Cg63-Pg1-Ps135 | Cg63-Pg1-Ps136 |
| Cg63-Pg1-Ps137 | Cg63-Pg1-Ps138 | Cg63-Pg1-Ps139 | Cg63-Pg1-Ps140 |
| Cg63-Pg1-Ps141 | Cg63-Pg1-Ps142 | Cg63-Pg1-Ps143 | Cg63-Pg1-Ps144 |
| Cg63-Pg1-Ps145 | Cg63-Pg1-Ps146 | Cg63-Pg1-Ps147 | Cg63-Pg1-Ps148 |
| Cg63-Pg1-Ps149 | Cg63-Pg1-Ps150 | Cg63-Pg1-Ps151 | Cg63-Pg1-Ps152 |
| Cg63-Pg1-Ps153 | Cg63-Pg1-Ps154 | Cg63-Pg1-Ps155 | Cg63-Pg1-Ps156 |
| Cg63-Pg1-Ps157 | Cg63-Pg1-Ps158 | Cg63-Pg1-Ps159 | Cg63-Pg1-Ps160 |
| Cg63-Pg1-Ps161 | Cg63-Pg1-Ps162 | Cg63-Pg1-Ps163 | Cg63-Pg1-Ps164 |
| Cg63-Pg1-Ps165 | Cg63-Pg1-Ps166 | Cg63-Pg1-Ps167 | Cg63-Pg1-Ps168 |
| Cg63-Pg1-Ps169 | Cg63-Pg1-Ps170 | Cg63-Pg1-Ps171 | Cg63-Pg1-Ps172 |
| Cg63-Pg1-Ps173 | Cg63-Pg1-Ps174 | Cg63-Pg1-Ps175 | Cg63-Pg1-Ps176 |
| Cg63-Pg1-Ps177 | Cg63-Pg1-Ps178 | Cg63-Pg1-Ps179 | Cg63-Pg1-Ps180 |
| Cg63-Pg1-Ps181 | Cg63-Pg1-Ps182 | Cg63-Pg1-Ps183 | Cg63-Pg1-Ps184 |
| Cg63-Pg1-Ps185 | Cg63-Pg1-Ps186 | Cg63-Pg1-Ps187 | Cg63-Pg1-Ps188 |
| Cg63-Pg1-Ps189 | Cg63-Pg1-Ps190 | Cg63-Pg1-Ps191 | Cg63-Pg1-Ps192 |
| Cg63-Pg1-Ps193 | Cg63-Pg1-Ps194 | Cg63-Pg1-Ps195 | Cg63-Pg1-Ps196 |
| Cg63-Pg1-Ps197 | Cg63-Pg1-Ps198 | Cg63-Pg1-Ps199 | Cg63-Pg1-Ps200 |
| Cg63-Pg1-Ps201 | Cg63-Pg1-Ps202 | Cg63-Pg1-Ps203 | Cg63-Pg1-Ps204 |
| Cg63-Pg1-Ps205 | Cg63-Pg1-Ps206 | Cg63-Pg1-Ps207 | Cg63-Pg1-Ps208 |
| Cg63-Pg1-Ps209 | Cg63-Pg1-Ps210 | Cg63-Pg1-Ps211 | Cg63-Pg1-Ps212 |
| Cg63-Pg1-Ps213 | Cg63-Pg1-Ps214 | Cg63-Pg1-Ps215 | Cg63-Pg1-Ps216 |
| Cg63-Pg1-Ps217 | Cg63-Pg1-Ps218 | Cg63-Pg1-Ps219 | Cg63-Pg1-Ps220 |
| Cg63-Pg1-Ps221 | Cg63-Pg1-Ps222 | Cg63-Pg1-Ps223 | Cg63-Pg1-Ps224 |
| Cg63-Pg1-Ps225 | Cg63-Pg1-Ps226 | Cg63-Pg1-Ps227 | Cg63-Pg1-Ps228 |
| Cg63-Pg1-Ps229 | Cg63-Pg1-Ps230 | Cg63-Pg1-Ps231 | Cg63-Pg1-Ps232 |
| Cg63-Pg1-Ps233 | Cg63-Pg1-Ps234 | Cg63-Pg1-Ps235 | Cg63-Pg1-Ps236 |
| Cg63-Pg1-Ps237 | Cg63-Pg1-Ps238 | Cg63-Pg1-Ps239 | Cg63-Pg1-Ps240 |
| Cg63-Pg1-Ps241 | Cg63-Pg1-Ps242 | Cg63-Pg1-Ps243 | |
| Cg64-Pg1-Ps1 | Cg64-Pg1-Ps2 | Cg64-Pg1-Ps3 | Cg64-Pg1-Ps4 |
| Cg64-Pg1-Ps5 | Cg64-Pg1-Ps6 | Cg64-Pg1-Ps7 | Cg64-Pg1-Ps8 |
| Cg64-Pg1-Ps9 | Cg64-Pg1-Ps10 | Cg64-Pg1-Ps11 | Cg64-Pg1-Ps12 |
| Cg64-Pg1-Ps13 | Cg64-Pg1-Ps14 | Cg64-Pg1-Ps15 | Cg64-Pg1-Ps16 |
| Cg64-Pg1-Ps17 | Cg64-Pg1-Ps18 | Cg64-Pg1-Ps19 | Cg64-Pg1-Ps20 |
| Cg64-Pg1-Ps21 | Cg64-Pg1-Ps22 | Cg64-Pg1-Ps23 | Cg64-Pg1-Ps24 |
| Cg64-Pg1-Ps25 | Cg64-Pg1-Ps26 | Cg64-Pg1-Ps27 | Cg64-Pg1-Ps28 |
| Cg64-Pg1-Ps29 | Cg64-Pg1-Ps30 | Cg64-Pg1-Ps31 | Cg64-Pg1-Ps32 |
| Cg64-Pg1-Ps33 | Cg64-Pg1-Ps34 | Cg64-Pg1-Ps35 | Cg64-Pg1-Ps36 |
| Cg64-Pg1-Ps37 | Cg64-Pg1-Ps38 | Cg64-Pg1-Ps39 | Cg64-Pg1-Ps40 |
| Cg64-Pg1-Ps41 | Cg64-Pg1-Ps42 | Cg64-Pg1-Ps43 | Cg64-Pg1-Ps44 |
| Cg64-Pg1-Ps45 | Cg64-Pg1-Ps46 | Cg64-Pg1-Ps47 | Cg64-Pg1-Ps48 |
| Cg64-Pg1-Ps49 | Cg64-Pg1-Ps50 | Cg64-Pg1-Ps51 | Cg64-Pg1-Ps52 |
| Cg64-Pg1-Ps53 | Cg64-Pg1-Ps54 | Cg64-Pg1-Ps55 | Cg64-Pg1-Ps56 |
| Cg64-Pg1-Ps57 | Cg64-Pg1-Ps58 | Cg64-Pg1-Ps59 | Cg64-Pg1-Ps60 |
| Cg64-Pg1-Ps61 | Cg64-Pg1-Ps62 | Cg64-Pg1-Ps63 | Cg64-Pg1-Ps64 |
| Cg64-Pg1-Ps65 | Cg64-Pg1-Ps66 | Cg64-Pg1-Ps67 | Cg64-Pg1-Ps68 |
| Cg64-Pg1-Ps69 | Cg64-Pg1-Ps70 | Cg64-Pg1-Ps71 | Cg64-Pg1-Ps72 |
| Cg64-Pg1-Ps73 | Cg64-Pg1-Ps74 | Cg64-Pg1-Ps75 | Cg64-Pg1-Ps76 |
| Cg64-Pg1-Ps77 | Cg64-Pg1-Ps78 | Cg64-Pg1-Ps79 | Cg64-Pg1-Ps80 |
| Cg64-Pg1-Ps81 | Cg64-Pg1-Ps82 | Cg64-Pg1-Ps83 | Cg64-Pg1-Ps84 |
| Cg64-Pg1-Ps85 | Cg64-Pg1-Ps86 | Cg64-Pg1-Ps87 | Cg64-Pg1-Ps88 |
| Cg64-Pg1-Ps89 | Cg64-Pg1-Ps90 | Cg64-Pg1-Ps91 | Cg64-Pg1-Ps92 |
| Cg64-Pg1-Ps93 | Cg64-Pg1-Ps94 | Cg64-Pg1-Ps95 | Cg64-Pg1-Ps96 |
| Cg64-Pg1-Ps97 | Cg64-Pg1-Ps98 | Cg64-Pg1-Ps99 | Cg64-Pg1-Ps100 |
| Cg64-Pg1-Ps101 | Cg64-Pg1-Ps102 | Cg64-Pg1-Ps103 | Cg64-Pg1-Ps104 |
| Cg64-Pg1-Ps105 | Cg64-Pg1-Ps106 | Cg64-Pg1-Ps107 | Cg64-Pg1-Ps108 |
| Cg64-Pg1-Ps109 | Cg64-Pg1-Ps110 | Cg64-Pg1-Ps111 | Cg64-Pg1-Ps112 |
| Cg64-Pg1-Ps113 | Cg64-Pg1-Ps114 | Cg64-Pg1-Ps115 | Cg64-Pg1-Ps116 |
| Cg64-Pg1-Ps117 | Cg64-Pg1-Ps118 | Cg64-Pg1-Ps119 | Cg64-Pg1-Ps120 |
| Cg64-Pg1-Ps121 | Cg64-Pg1-Ps122 | Cg64-Pg1-Ps123 | Cg64-Pg1-Ps124 |
| Cg64-Pg1-Ps125 | Cg64-Pg1-Ps126 | Cg64-Pg1-Ps127 | Cg64-Pg1-Ps128 |
| Cg64-Pg1-Ps129 | Cg64-Pg1-Ps130 | Cg64-Pg1-Ps131 | Cg64-Pg1-Ps132 |
| Cg64-Pg1-Ps133 | Cg64-Pg1-Ps134 | Cg64-Pg1-Ps135 | Cg64-Pg1-Ps136 |
| Cg64-Pg1-Ps137 | Cg64-Pg1-Ps138 | Cg64-Pg1-Ps139 | Cg64-Pg1-Ps140 |
| Cg64-Pg1-Ps141 | Cg64-Pg1-Ps142 | Cg64-Pg1-Ps143 | Cg64-Pg1-Ps144 |
| Cg64-Pg1-Ps145 | Cg64-Pg1-Ps146 | Cg64-Pg1-Ps147 | Cg64-Pg1-Ps148 |
| Cg64-Pg1-Ps149 | Cg64-Pg1-Ps150 | Cg64-Pg1-Ps151 | Cg64-Pg1-Ps152 |
| Cg64-Pg1-Ps153 | Cg64-Pg1-Ps154 | Cg64-Pg1-Ps155 | Cg64-Pg1-Ps156 |
| Cg64-Pg1-Ps157 | Cg64-Pg1-Ps158 | Cg64-Pg1-Ps159 | Cg64-Pg1-Ps160 |
| Cg64-Pg1-Ps161 | Cg64-Pg1-Ps162 | Cg64-Pg1-Ps163 | Cg64-Pg1-Ps164 |
| Cg64-Pg1-Ps165 | Cg64-Pg1-Ps166 | Cg64-Pg1-Ps167 | Cg64-Pg1-Ps168 |
| Cg64-Pg1-Ps169 | Cg64-Pg1-Ps170 | Cg64-Pg1-Ps171 | Cg64-Pg1-Ps172 |
| Cg64-Pg1-Ps173 | Cg64-Pg1-Ps174 | Cg64-Pg1-Ps175 | Cg64-Pg1-Ps176 |
| Cg64-Pg1-Ps177 | Cg64-Pg1-Ps178 | Cg64-Pg1-Ps179 | Cg64-Pg1-Ps180 |
| Cg64-Pg1-Ps181 | Cg64-Pg1-Ps182 | Cg64-Pg1-Ps183 | Cg64-Pg1-Ps184 |
| Cg64-Pg1-Ps185 | Cg64-Pg1-Ps186 | Cg64-Pg1-Ps187 | Cg64-Pg1-Ps188 |
| Cg64-Pg1-Ps189 | Cg64-Pg1-Ps190 | Cg64-Pg1-Ps191 | Cg64-Pg1-Ps192 |
| Cg64-Pg1-Ps193 | Cg64-Pg1-Ps194 | Cg64-Pg1-Ps195 | Cg64-Pg1-Ps196 |
| Cg64-Pg1-Ps197 | Cg64-Pg1-Ps198 | Cg64-Pg1-Ps199 | Cg64-Pg1-Ps200 |
| Cg64-Pg1-Ps201 | Cg64-Pg1-Ps202 | Cg64-Pg1-Ps203 | Cg64-Pg1-Ps204 |
| Cg64-Pg1-Ps205 | Cg64-Pg1-Ps206 | Cg64-Pg1-Ps207 | Cg64-Pg1-Ps208 |
| Cg64-Pg1-Ps209 | Cg64-Pg1-Ps210 | Cg64-Pg1-Ps211 | Cg64-Pg1-Ps212 |
| Cg64-Pg1-Ps213 | Cg64-Pg1-Ps214 | Cg64-Pg1-Ps215 | Cg64-Pg1-Ps216 |
| Cg64-Pg1-Ps217 | Cg64-Pg1-Ps218 | Cg64-Pg1-Ps219 | Cg64-Pg1-Ps220 |
| Cg64-Pg1-Ps221 | Cg64-Pg1-Ps222 | Cg64-Pg1-Ps223 | Cg64-Pg1-Ps224 |
| Cg64-Pg1-Ps225 | Cg64-Pg1-Ps226 | Cg64-Pg1-Ps227 | Cg64-Pg1-Ps228 |
| Cg64-Pg1-Ps229 | Cg64-Pg1-Ps230 | Cg64-Pg1-Ps231 | Cg64-Pg1-Ps232 |
| Cg64-Pg1-Ps233 | Cg64-Pg1-Ps234 | Cg64-Pg1-Ps235 | Cg64-Pg1-Ps236 |
| Cg64-Pg1-Ps237 | Cg64-Pg1-Ps238 | Cg64-Pg1-Ps239 | Cg64-Pg1-Ps240 |
| Cg64-Pg1-Ps241 | Cg64-Pg1-Ps242 | Cg64-Pg1-Ps243 | |
| Cg65-Pg1-Ps1 | Cg65-Pg1-Ps2 | Cg65-Pg1-Ps3 | Cg65-Pg1-Ps4 |
| Cg65-Pg1-Ps5 | Cg65-Pg1-Ps6 | Cg65-Pg1-Ps7 | Cg65-Pg1-Ps8 |
| Cg65-Pg1-Ps9 | Cg65-Pg1-Ps10 | Cg65-Pg1-Ps11 | Cg65-Pg1-Ps12 |
| Cg65-Pg1-Ps13 | Cg65-Pg1-Ps14 | Cg65-Pg1-Ps15 | Cg65-Pg1-Ps16 |
| Cg65-Pg1-Ps17 | Cg65-Pg1-Ps18 | Cg65-Pg1-Ps19 | Cg65-Pg1-Ps20 |
| Cg65-Pg1-Ps21 | Cg65-Pg1-Ps22 | Cg65-Pg1-Ps23 | Cg65-Pg1-Ps24 |
| Cg65-Pg1-Ps25 | Cg65-Pg1-Ps26 | Cg65-Pg1-Ps27 | Cg65-Pg1-Ps28 |
| Cg65-Pg1-Ps29 | Cg65-Pg1-Ps30 | Cg65-Pg1-Ps31 | Cg65-Pg1-Ps32 |
| Cg65-Pg1-Ps33 | Cg65-Pg1-Ps34 | Cg65-Pg1-Ps35 | Cg65-Pg1-Ps36 |
| Cg65-Pg1-Ps37 | Cg65-Pg1-Ps38 | Cg65-Pg1-Ps39 | Cg65-Pg1-Ps40 |
| Cg65-Pg1-Ps41 | Cg65-Pg1-Ps42 | Cg65-Pg1-Ps43 | Cg65-Pg1-Ps44 |
| Cg65-Pg1-Ps45 | Cg65-Pg1-Ps46 | Cg65-Pg1-Ps47 | Cg65-Pg1-Ps48 |
| Cg65-Pg1-Ps49 | Cg65-Pg1-Ps50 | Cg65-Pg1-Ps51 | Cg65-Pg1-Ps52 |
| Cg65-Pg1-Ps53 | Cg65-Pg1-Ps54 | Cg65-Pg1-Ps55 | Cg65-Pg1-Ps56 |
| Cg65-Pg1-Ps57 | Cg65-Pg1-Ps58 | Cg65-Pg1-Ps59 | Cg65-Pg1-Ps60 |
| Cg65-Pg1-Ps61 | Cg65-Pg1-Ps62 | Cg65-Pg1-Ps63 | Cg65-Pg1-Ps64 |
| Cg65-Pg1-Ps65 | Cg65-Pg1-Ps66 | Cg65-Pg1-Ps67 | Cg65-Pg1-Ps68 |
| Cg65-Pg1-Ps69 | Cg65-Pg1-Ps70 | Cg65-Pg1-Ps71 | Cg65-Pg1-Ps72 |
| Cg65-Pg1-Ps73 | Cg65-Pg1-Ps74 | Cg65-Pg1-Ps75 | Cg65-Pg1-Ps76 |
| Cg65-Pg1-Ps77 | Cg65-Pg1-Ps78 | Cg65-Pg1-Ps79 | Cg65-Pg1-Ps80 |
| Cg65-Pg1-Ps81 | Cg65-Pg1-Ps82 | Cg65-Pg1-Ps83 | Cg65-Pg1-Ps84 |
| Cg65-Pg1-Ps85 | Cg65-Pg1-Ps86 | Cg65-Pg1-Ps87 | Cg65-Pg1-Ps88 |
| Cg65-Pg1-Ps89 | Cg65-Pg1-Ps90 | Cg65-Pg1-Ps91 | Cg65-Pg1-Ps92 |
| Cg65-Pg1-Ps93 | Cg65-Pg1-Ps94 | Cg65-Pg1-Ps95 | Cg65-Pg1-Ps96 |
| Cg65-Pg1-Ps97 | Cg65-Pg1-Ps98 | Cg65-Pg1-Ps99 | Cg65-Pg1-Ps100 |
| Cg65-Pg1-Ps101 | Cg65-Pg1-Ps102 | Cg65-Pg1-Ps103 | Cg65-Pg1-Ps104 |
| Cg65-Pg1-Ps105 | Cg65-Pg1-Ps106 | Cg65-Pg1-Ps107 | Cg65-Pg1-Ps108 |
| Cg65-Pg1-Ps109 | Cg65-Pg1-Ps110 | Cg65-Pg1-Ps111 | Cg65-Pg1-Ps112 |
| Cg65-Pg1-Ps113 | Cg65-Pg1-Ps114 | Cg65-Pg1-Ps115 | Cg65-Pg1-Ps116 |
| Cg65-Pg1-Ps117 | Cg65-Pg1-Ps118 | Cg65-Pg1-Ps119 | Cg65-Pg1-Ps120 |
| Cg65-Pg1-Ps121 | Cg65-Pg1-Ps122 | Cg65-Pg1-Ps123 | Cg65-Pg1-Ps124 |
| Cg65-Pg1-Ps125 | Cg65-Pg1-Ps126 | Cg65-Pg1-Ps127 | Cg65-Pg1-Ps128 |
| Cg65-Pg1-Ps129 | Cg65-Pg1-Ps130 | Cg65-Pg1-Ps131 | Cg65-Pg1-Ps132 |
| Cg65-Pg1-Ps133 | Cg65-Pg1-Ps134 | Cg65-Pg1-Ps135 | Cg65-Pg1-Ps136 |
| Cg65-Pg1-Ps137 | Cg65-Pg1-Ps138 | Cg65-Pg1-Ps139 | Cg65-Pg1-Ps140 |
| Cg65-Pg1-Ps141 | Cg65-Pg1-Ps142 | Cg65-Pg1-Ps143 | Cg65-Pg1-Ps144 |
| Cg65-Pg1-Ps145 | Cg65-Pg1-Ps146 | Cg65-Pg1-Ps147 | Cg65-Pg1-Ps148 |
| Cg65-Pg1-Ps149 | Cg65-Pg1-Ps150 | Cg65-Pg1-Ps151 | Cg65-Pg1-Ps152 |
| Cg65-Pg1-Ps153 | Cg65-Pg1-Ps154 | Cg65-Pg1-Ps155 | Cg65-Pg1-Ps156 |
| Cg65-Pg1-Ps157 | Cg65-Pg1-Ps158 | Cg65-Pg1-Ps159 | Cg65-Pg1-Ps160 |
| Cg65-Pg1-Ps161 | Cg65-Pg1-Ps162 | Cg65-Pg1-Ps163 | Cg65-Pg1-Ps164 |
| Cg65-Pg1-Ps165 | Cg65-Pg1-Ps166 | Cg65-Pg1-Ps167 | Cg65-Pg1-Ps168 |
| Cg65-Pg1-Ps169 | Cg65-Pg1-Ps170 | Cg65-Pg1-Ps171 | Cg65-Pg1-Ps172 |
| Cg65-Pg1-Ps173 | Cg65-Pg1-Ps174 | Cg65-Pg1-Ps175 | Cg65-Pg1-Ps176 |
| Cg65-Pg1-Ps177 | Cg65-Pg1-Ps178 | Cg65-Pg1-Ps179 | Cg65-Pg1-Ps180 |
| Cg65-Pg1-Ps181 | Cg65-Pg1-Ps182 | Cg65-Pg1-Ps183 | Cg65-Pg1-Ps184 |
| Cg65-Pg1-Ps185 | Cg65-Pg1-Ps186 | Cg65-Pg1-Ps187 | Cg65-Pg1-Ps188 |
| Cg65-Pg1-Ps189 | Cg65-Pg1-Ps190 | Cg65-Pg1-Ps191 | Cg65-Pg1-Ps192 |
| Cg65-Pg1-Ps193 | Cg65-Pg1-Ps194 | Cg65-Pg1-Ps195 | Cg65-Pg1-Ps196 |
| Cg65-Pg1-Ps197 | Cg65-Pg1-Ps198 | Cg65-Pg1-Ps199 | Cg65-Pg1-Ps200 |
| Cg65-Pg1-Ps201 | Cg65-Pg1-Ps202 | Cg65-Pg1-Ps203 | Cg65-Pg1-Ps204 |
| Cg65-Pg1-Ps205 | Cg65-Pg1-Ps206 | Cg65-Pg1-Ps207 | Cg65-Pg1-Ps208 |
| Cg65-Pg1-Ps209 | Cg65-Pg1-Ps210 | Cg65-Pg1-Ps211 | Cg65-Pg1-Ps212 |
| Cg65-Pg1-Ps213 | Cg65-Pg1-Ps214 | Cg65-Pg1-Ps215 | Cg65-Pg1-Ps216 |
| Cg65-Pg1-Ps217 | Cg65-Pg1-Ps218 | Cg65-Pg1-Ps219 | Cg65-Pg1-Ps220 |
| Cg65-Pg1-Ps221 | Cg65-Pg1-Ps222 | Cg65-Pg1-Ps223 | Cg65-Pg1-Ps224 |
| Cg65-Pg1-Ps225 | Cg65-Pg1-Ps226 | Cg65-Pg1-Ps227 | Cg65-Pg1-Ps228 |
| Cg65-Pg1-Ps229 | Cg65-Pg1-Ps230 | Cg65-Pg1-Ps231 | Cg65-Pg1-Ps232 |
| Cg65-Pg1-Ps233 | Cg65-Pg1-Ps234 | Cg65-Pg1-Ps235 | Cg65-Pg1-Ps236 |
| Cg65-Pg1-Ps237 | Cg65-Pg1-Ps238 | Cg65-Pg1-Ps239 | Cg65-Pg1-Ps240 |
| Cg65-Pg1-Ps241 | Cg65-Pg1-Ps242 | Cg65-Pg1-Ps243 | |
| Cg67-Pg1-Ps1 | Cg67-Pg1-Ps2 | Cg67-Pg1-Ps3 | Cg67-Pg1-Ps4 |
| Cg67-Pg1-Ps5 | Cg67-Pg1-Ps6 | Cg67-Pg1-Ps7 | Cg67-Pg1-Ps8 |
| Cg67-Pg1-Ps9 | Cg67-Pg1-Ps10 | Cg67-Pg1-Ps11 | Cg67-Pg1-Ps12 |
| Cg67-Pg1-Ps13 | Cg67-Pg1-Ps14 | Cg67-Pg1-Ps15 | Cg67-Pg1-Ps16 |
| Cg67-Pg1-Ps17 | Cg67-Pg1-Ps18 | Cg67-Pg1-Ps19 | Cg67-Pg1-Ps20 |
| Cg67-Pg1-Ps21 | Cg67-Pg1-Ps22 | Cg67-Pg1-Ps23 | Cg67-Pg1-Ps24 |
| Cg67-Pg1-Ps25 | Cg67-Pg1-Ps26 | Cg67-Pg1-Ps27 | Cg67-Pg1-Ps28 |
| Cg67-Pg1-Ps29 | Cg67-Pg1-Ps30 | Cg67-Pg1-Ps31 | Cg67-Pg1-Ps32 |
| Cg67-Pg1-Ps33 | Cg67-Pg1-Ps34 | Cg67-Pg1-Ps35 | Cg67-Pg1-Ps36 |
| Cg67-Pg1-Ps37 | Cg67-Pg1-Ps38 | Cg67-Pg1-Ps39 | Cg67-Pg1-Ps40 |
| Cg67-Pg1-Ps41 | Cg67-Pg1-Ps42 | Cg67-Pg1-Ps43 | Cg67-Pg1-Ps44 |
| Cg67-Pg1-Ps45 | Cg67-Pg1-Ps46 | Cg67-Pg1-Ps47 | Cg67-Pg1-Ps48 |
| Cg67-Pg1-Ps49 | Cg67-Pg1-Ps50 | Cg67-Pg1-Ps51 | Cg67-Pg1-Ps52 |
| Cg67-Pg1-Ps53 | Cg67-Pg1-Ps54 | Cg67-Pg1-Ps55 | Cg67-Pg1-Ps56 |
| Cg67-Pg1-Ps57 | Cg67-Pg1-Ps58 | Cg67-Pg1-Ps59 | Cg67-Pg1-Ps60 |
| Cg67-Pg1-Ps61 | Cg67-Pg1-Ps62 | Cg67-Pg1-Ps63 | Cg67-Pg1-Ps64 |
| Cg67-Pg1-Ps65 | Cg67-Pg1-Ps66 | Cg67-Pg1-Ps67 | Cg67-Pg1-Ps68 |
| Cg67-Pg1-Ps69 | Cg67-Pg1-Ps70 | Cg67-Pg1-Ps71 | Cg67-Pg1-Ps72 |
| Cg67-Pg1-Ps73 | Cg67-Pg1-Ps74 | Cg67-Pg1-Ps75 | Cg67-Pg1-Ps76 |
| Cg67-Pg1-Ps77 | Cg67-Pg1-Ps78 | Cg67-Pg1-Ps79 | Cg67-Pg1-Ps80 |
| Cg67-Pg1-Ps81 | Cg67-Pg1-Ps82 | Cg67-Pg1-Ps83 | Cg67-Pg1-Ps84 |
| Cg67-Pg1-Ps85 | Cg67-Pg1-Ps86 | Cg67-Pg1-Ps87 | Cg67-Pg1-Ps88 |
| Cg67-Pg1-Ps89 | Cg67-Pg1-Ps90 | Cg67-Pg1-Ps91 | Cg67-Pg1-Ps92 |
| Cg67-Pg1-Ps93 | Cg67-Pg1-Ps94 | Cg67-Pg1-Ps95 | Cg67-Pg1-Ps96 |
| Cg67-Pg1-Ps97 | Cg67-Pg1-Ps98 | Cg67-Pg1-Ps99 | Cg67-Pg1-Ps100 |
| Cg67-Pg1-Ps101 | Cg67-Pg1-Ps102 | Cg67-Pg1-Ps103 | Cg67-Pg1-Ps104 |
| Cg67-Pg1-Ps105 | Cg67-Pg1-Ps106 | Cg67-Pg1-Ps107 | Cg67-Pg1-Ps108 |
| Cg67-Pg1-Ps109 | Cg67-Pg1-Ps110 | Cg67-Pg1-Ps111 | Cg67-Pg1-Ps112 |
| Cg67-Pg1-Ps113 | Cg67-Pg1-Ps114 | Cg67-Pg1-Ps115 | Cg67-Pg1-Ps116 |
| Cg67-Pg1-Ps117 | Cg67-Pg1-Ps118 | Cg67-Pg1-Ps119 | Cg67-Pg1-Ps120 |
| Cg67-Pg1-Ps121 | Cg67-Pg1-Ps122 | Cg67-Pg1-Ps123 | Cg67-Pg1-Ps124 |
| Cg67-Pg1-Ps125 | Cg67-Pg1-Ps126 | Cg67-Pg1-Ps127 | Cg67-Pg1-Ps128 |
| Cg67-Pg1-Ps129 | Cg67-Pg1-Ps130 | Cg67-Pg1-Ps131 | Cg67-Pg1-Ps132 |
| Cg67-Pg1-Ps133 | Cg67-Pg1-Ps134 | Cg67-Pg1-Ps135 | Cg67-Pg1-Ps136 |
| Cg67-Pg1-Ps137 | Cg67-Pg1-Ps138 | Cg67-Pg1-Ps139 | Cg67-Pg1-Ps140 |
| Cg67-Pg1-Ps141 | Cg67-Pg1-Ps142 | Cg67-Pg1-Ps143 | Cg67-Pg1-Ps144 |
| Cg67-Pg1-Ps145 | Cg67-Pg1-Ps146 | Cg67-Pg1-Ps147 | Cg67-Pg1-Ps148 |
| Cg67-Pg1-Ps149 | Cg67-Pg1-Ps150 | Cg67-Pg1-Ps151 | Cg67-Pg1-Ps152 |
| Cg67-Pg1-Ps153 | Cg67-Pg1-Ps154 | Cg67-Pg1-Ps155 | Cg67-Pg1-Ps156 |
| Cg67-Pg1-Ps157 | Cg67-Pg1-Ps158 | Cg67-Pg1-Ps159 | Cg67-Pg1-Ps160 |
| Cg67-Pg1-Ps161 | Cg67-Pg1-Ps162 | Cg67-Pg1-Ps163 | Cg67-Pg1-Ps164 |
| Cg67-Pg1-Ps165 | Cg67-Pg1-Ps166 | Cg67-Pg1-Ps167 | Cg67-Pg1-Ps168 |
| Cg67-Pg1-Ps169 | Cg67-Pg1-Ps170 | Cg67-Pg1-Ps171 | Cg67-Pg1-Ps172 |
| Cg67-Pg1-Ps173 | Cg67-Pg1-Ps174 | Cg67-Pg1-Ps175 | Cg67-Pg1-Ps176 |
| Cg67-Pg1-Ps177 | Cg67-Pg1-Ps178 | Cg67-Pg1-Ps179 | Cg67-Pg1-Ps180 |
| Cg67-Pg1-Ps181 | Cg67-Pg1-Ps182 | Cg67-Pg1-Ps183 | Cg67-Pg1-Ps184 |
| Cg67-Pg1-Ps185 | Cg67-Pg1-Ps186 | Cg67-Pg1-Ps187 | Cg67-Pg1-Ps188 |
| Cg67-Pg1-Ps189 | Cg67-Pg1-Ps190 | Cg67-Pg1-Ps191 | Cg67-Pg1-Ps192 |
| Cg67-Pg1-Ps193 | Cg67-Pg1-Ps194 | Cg67-Pg1-Ps195 | Cg67-Pg1-Ps196 |
| Cg67-Pg1-Ps197 | Cg67-Pg1-Ps198 | Cg67-Pg1-Ps199 | Cg67-Pg1-Ps200 |
| Cg67-Pg1-Ps201 | Cg67-Pg1-Ps202 | Cg67-Pg1-Ps203 | Cg67-Pg1-Ps204 |
| Cg67-Pg1-Ps205 | Cg67-Pg1-Ps206 | Cg67-Pg1-Ps207 | Cg67-Pg1-Ps208 |
| Cg67-Pg1-Ps209 | Cg67-Pg1-Ps210 | Cg67-Pg1-Ps211 | Cg67-Pg1-Ps212 |
| Cg67-Pg1-Ps213 | Cg67-Pg1-Ps214 | Cg67-Pg1-Ps215 | Cg67-Pg1-Ps216 |
| Cg67-Pg1-Ps217 | Cg67-Pg1-Ps218 | Cg67-Pg1-Ps219 | Cg67-Pg1-Ps220 |
| Cg67-Pg1-Ps221 | Cg67-Pg1-Ps222 | Cg67-Pg1-Ps223 | Cg67-Pg1-Ps224 |
| Cg67-Pg1-Ps225 | Cg67-Pg1-Ps226 | Cg67-Pg1-Ps227 | Cg67-Pg1-Ps228 |
| Cg67-Pg1-Ps229 | Cg67-Pg1-Ps230 | Cg67-Pg1-Ps231 | Cg67-Pg1-Ps232 |
| Cg67-Pg1-Ps233 | Cg67-Pg1-Ps234 | Cg67-Pg1-Ps235 | Cg67-Pg1-Ps236 |
| Cg67-Pg1-Ps237 | Cg67-Pg1-Ps238 | Cg67-Pg1-Ps239 | Cg67-Pg1-Ps240 |
| Cg67-Pg1-Ps241 | Cg67-Pg1-Ps242 | Cg67-Pg1-Ps243 | |
| Cg68-Pg1-Ps1 | Cg68-Pg1-Ps2 | Cg68-Pg1-Ps3 | Cg68-Pg1-Ps4 |
| Cg68-Pg1-Ps5 | Cg68-Pg1-Ps6 | Cg68-Pg1-Ps7 | Cg68-Pg1-Ps8 |
| Cg68-Pg1-Ps9 | Cg68-Pg1-Ps10 | Cg68-Pg1-Ps11 | Cg68-Pg1-Ps12 |
| Cg68-Pg1-Ps13 | Cg68-Pg1-Ps14 | Cg68-Pg1-Ps15 | Cg68-Pg1-Ps16 |
| Cg68-Pg1-Ps17 | Cg68-Pg1-Ps18 | Cg68-Pg1-Ps19 | Cg68-Pg1-Ps20 |
| Cg68-Pg1-Ps21 | Cg68-Pg1-Ps22 | Cg68-Pg1-Ps23 | Cg68-Pg1-Ps24 |
| Cg68-Pg1-Ps25 | Cg68-Pg1-Ps26 | Cg68-Pg1-Ps27 | Cg68-Pg1-Ps28 |
| Cg68-Pg1-Ps29 | Cg68-Pg1-Ps30 | Cg68-Pg1-Ps31 | Cg68-Pg1-Ps32 |
| Cg68-Pg1-Ps33 | Cg68-Pg1-Ps34 | Cg68-Pg1-Ps35 | Cg68-Pg1-Ps36 |
| Cg68-Pg1-Ps37 | Cg68-Pg1-Ps38 | Cg68-Pg1-Ps39 | Cg68-Pg1-Ps40 |
| Cg68-Pg1-Ps41 | Cg68-Pg1-Ps42 | Cg68-Pg1-Ps43 | Cg68-Pg1-Ps44 |
| Cg68-Pg1-Ps45 | Cg68-Pg1-Ps46 | Cg68-Pg1-Ps47 | Cg68-Pg1-Ps48 |
| Cg68-Pg1-Ps49 | Cg68-Pg1-Ps50 | Cg68-Pg1-Ps51 | Cg68-Pg1-Ps52 |
| Cg68-Pg1-Ps53 | Cg68-Pg1-Ps54 | Cg68-Pg1-Ps55 | Cg68-Pg1-Ps56 |
| Cg68-Pg1-Ps57 | Cg68-Pg1-Ps58 | Cg68-Pg1-Ps59 | Cg68-Pg1-Ps60 |
| Cg68-Pg1-Ps61 | Cg68-Pg1-Ps62 | Cg68-Pg1-Ps63 | Cg68-Pg1-Ps64 |
| Cg68-Pg1-Ps65 | Cg68-Pg1-Ps66 | Cg68-Pg1-Ps67 | Cg68-Pg1-Ps68 |
| Cg68-Pg1-Ps69 | Cg68-Pg1-Ps70 | Cg68-Pg1-Ps71 | Cg68-Pg1-Ps72 |
| Cg68-Pg1-Ps73 | Cg68-Pg1-Ps74 | Cg68-Pg1-Ps75 | Cg68-Pg1-Ps76 |
| Cg68-Pg1-Ps77 | Cg68-Pg1-Ps78 | Cg68-Pg1-Ps79 | Cg68-Pg1-Ps80 |
| Cg68-Pg1-Ps81 | Cg68-Pg1-Ps82 | Cg68-Pg1-Ps83 | Cg68-Pg1-Ps84 |
| Cg68-Pg1-Ps85 | Cg68-Pg1-Ps86 | Cg68-Pg1-Ps87 | Cg68-Pg1-Ps88 |
| Cg68-Pg1-Ps89 | Cg68-Pg1-Ps90 | Cg68-Pg1-Ps91 | Cg68-Pg1-Ps92 |
| Cg68-Pg1-Ps93 | Cg68-Pg1-Ps94 | Cg68-Pg1-Ps95 | Cg68-Pg1-Ps96 |
| Cg68-Pg1-Ps97 | Cg68-Pg1-Ps98 | Cg68-Pg1-Ps99 | Cg68-Pg1-Ps100 |
| Cg68-Pg1-Ps101 | Cg68-Pg1-Ps102 | Cg68-Pg1-Ps103 | Cg68-Pg1-Ps104 |
| Cg68-Pg1-Ps105 | Cg68-Pg1-Ps106 | Cg68-Pg1-Ps107 | Cg68-Pg1-Ps108 |
| Cg68-Pg1-Ps109 | Cg68-Pg1-Ps110 | Cg68-Pg1-Ps111 | Cg68-Pg1-Ps112 |
| Cg68-Pg1-Ps113 | Cg68-Pg1-Ps114 | Cg68-Pg1-Ps115 | Cg68-Pg1-Ps116 |
| Cg68-Pg1-Ps117 | Cg68-Pg1-Ps118 | Cg68-Pg1-Ps119 | Cg68-Pg1-Ps120 |
| Cg68-Pg1-Ps121 | Cg68-Pg1-Ps122 | Cg68-Pg1-Ps123 | Cg68-Pg1-Ps124 |
| Cg68-Pg1-Ps125 | Cg68-Pg1-Ps126 | Cg68-Pg1-Ps127 | Cg68-Pg1-Ps128 |
| Cg68-Pg1-Ps129 | Cg68-Pg1-Ps130 | Cg68-Pg1-Ps131 | Cg68-Pg1-Ps132 |
| Cg68-Pg1-Ps133 | Cg68-Pg1-Ps134 | Cg68-Pg1-Ps135 | Cg68-Pg1-Ps136 |
| Cg68-Pg1-Ps137 | Cg68-Pg1-Ps138 | Cg68-Pg1-Ps139 | Cg68-Pg1-Ps140 |
| Cg68-Pg1-Ps141 | Cg68-Pg1-Ps142 | Cg68-Pg1-Ps143 | Cg68-Pg1-Ps144 |
| Cg68-Pg1-Ps145 | Cg68-Pg1-Ps146 | Cg68-Pg1-Ps147 | Cg68-Pg1-Ps148 |
| Cg68-Pg1-Ps149 | Cg68-Pg1-Ps150 | Cg68-Pg1-Ps151 | Cg68-Pg1-Ps152 |
| Cg68-Pg1-Ps153 | Cg68-Pg1-Ps154 | Cg68-Pg1-Ps155 | Cg68-Pg1-Ps156 |
| Cg68-Pg1-Ps157 | Cg68-Pg1-Ps158 | Cg68-Pg1-Ps159 | Cg68-Pg1-Ps160 |
| Cg68-Pg1-Ps161 | Cg68-Pg1-Ps162 | Cg68-Pg1-Ps163 | Cg68-Pg1-Ps164 |
| Cg68-Pg1-Ps165 | Cg68-Pg1-Ps166 | Cg68-Pg1-Ps167 | Cg68-Pg1-Ps168 |
| Cg68-Pg1-Ps169 | Cg68-Pg1-Ps170 | Cg68-Pg1-Ps171 | Cg68-Pg1-Ps172 |
| Cg68-Pg1-Ps173 | Cg68-Pg1-Ps174 | Cg68-Pg1-Ps175 | Cg68-Pg1-Ps176 |
| Cg68-Pg1-Ps177 | Cg68-Pg1-Ps178 | Cg68-Pg1-Ps179 | Cg68-Pg1-Ps180 |
| Cg68-Pg1-Ps181 | Cg68-Pg1-Ps182 | Cg68-Pg1-Ps183 | Cg68-Pg1-Ps184 |
| Cg68-Pg1-Ps185 | Cg68-Pg1-Ps186 | Cg68-Pg1-Ps187 | Cg68-Pg1-Ps188 |
| Cg68-Pg1-Ps189 | Cg68-Pg1-Ps190 | Cg68-Pg1-Ps191 | Cg68-Pg1-Ps192 |
| Cg68-Pg1-Ps193 | Cg68-Pg1-Ps194 | Cg68-Pg1-Ps195 | Cg68-Pg1-Ps196 |
| Cg68-Pg1-Ps197 | Cg68-Pg1-Ps198 | Cg68-Pg1-Ps199 | Cg68-Pg1-Ps200 |
| Cg68-Pg1-Ps201 | Cg68-Pg1-Ps202 | Cg68-Pg1-Ps203 | Cg68-Pg1-Ps204 |
| Cg68-Pg1-Ps205 | Cg68-Pg1-Ps206 | Cg68-Pg1-Ps207 | Cg68-Pg1-Ps208 |
| Cg68-Pg1-Ps209 | Cg68-Pg1-Ps210 | Cg68-Pg1-Ps211 | Cg68-Pg1-Ps212 |
| Cg68-Pg1-Ps213 | Cg68-Pg1-Ps214 | Cg68-Pg1-Ps215 | Cg68-Pg1-Ps216 |
| Cg68-Pg1-Ps217 | Cg68-Pg1-Ps218 | Cg68-Pg1-Ps219 | Cg68-Pg1-Ps220 |
| Cg68-Pg1-Ps221 | Cg68-Pg1-Ps222 | Cg68-Pg1-Ps223 | Cg68-Pg1-Ps224 |
| Cg68-Pg1-Ps225 | Cg68-Pg1-Ps226 | Cg68-Pg1-Ps227 | Cg68-Pg1-Ps228 |
| Cg68-Pg1-Ps229 | Cg68-Pg1-Ps230 | Cg68-Pg1-Ps231 | Cg68-Pg1-Ps232 |
| Cg68-Pg1-Ps233 | Cg68-Pg1-Ps234 | Cg68-Pg1-Ps235 | Cg68-Pg1-Ps236 |
| Cg68-Pg1-Ps237 | Cg68-Pg1-Ps238 | Cg68-Pg1-Ps239 | Cg68-Pg1-Ps240 |
| Cg68-Pg1-Ps241 | Cg68-Pg1-Ps242 | Cg68-Pg1-Ps243 | |
| Cg69-Pg1-Ps1 | Cg69-Pg1-Ps2 | Cg69-Pg1-Ps3 | Cg69-Pg1-Ps4 |
| Cg69-Pg1-Ps5 | Cg69-Pg1-Ps6 | Cg69-Pg1-Ps7 | Cg69-Pg1-Ps8 |
| Cg69-Pg1-Ps9 | Cg69-Pg1-Ps10 | Cg69-Pg1-Ps11 | Cg69-Pg1-Ps12 |
| Cg69-Pg1-Ps13 | Cg69-Pg1-Ps14 | Cg69-Pg1-Ps15 | Cg69-Pg1-Ps16 |
| Cg69-Pg1-Ps17 | Cg69-Pg1-Ps18 | Cg69-Pg1-Ps19 | Cg69-Pg1-Ps20 |
| Cg69-Pg1-Ps21 | Cg69-Pg1-Ps22 | Cg69-Pg1-Ps23 | Cg69-Pg1-Ps24 |
| Cg69-Pg1-Ps25 | Cg69-Pg1-Ps26 | Cg69-Pg1-Ps27 | Cg69-Pg1-Ps28 |
| Cg69-Pg1-Ps29 | Cg69-Pg1-Ps30 | Cg69-Pg1-Ps31 | Cg69-Pg1-Ps32 |
| Cg69-Pg1-Ps33 | Cg69-Pg1-Ps34 | Cg69-Pg1-Ps35 | Cg69-Pg1-Ps36 |
| Cg69-Pg1-Ps37 | Cg69-Pg1-Ps38 | Cg69-Pg1-Ps39 | Cg69-Pg1-Ps40 |
| Cg69-Pg1-Ps41 | Cg69-Pg1-Ps42 | Cg69-Pg1-Ps43 | Cg69-Pg1-Ps44 |
| Cg69-Pg1-Ps45 | Cg69-Pg1-Ps46 | Cg69-Pg1-Ps47 | Cg69-Pg1-Ps48 |
| Cg69-Pg1-Ps49 | Cg69-Pg1-Ps50 | Cg69-Pg1-Ps51 | Cg69-Pg1-Ps52 |
| Cg69-Pg1-Ps53 | Cg69-Pg1-Ps54 | Cg69-Pg1-Ps55 | Cg69-Pg1-Ps56 |
| Cg69-Pg1-Ps57 | Cg69-Pg1-Ps58 | Cg69-Pg1-Ps59 | Cg69-Pg1-Ps60 |
| Cg69-Pg1-Ps61 | Cg69-Pg1-Ps62 | Cg69-Pg1-Ps63 | Cg69-Pg1-Ps64 |
| Cg69-Pg1-Ps65 | Cg69-Pg1-Ps66 | Cg69-Pg1-Ps67 | Cg69-Pg1-Ps68 |
| Cg69-Pg1-Ps69 | Cg69-Pg1-Ps70 | Cg69-Pg1-Ps71 | Cg69-Pg1-Ps72 |
| Cg69-Pg1-Ps73 | Cg69-Pg1-Ps74 | Cg69-Pg1-Ps75 | Cg69-Pg1-Ps76 |
| Cg69-Pg1-Ps77 | Cg69-Pg1-Ps78 | Cg69-Pg1-Ps79 | Cg69-Pg1-Ps80 |
| Cg69-Pg1-Ps81 | Cg69-Pg1-Ps82 | Cg69-Pg1-Ps83 | Cg69-Pg1-Ps84 |
| Cg69-Pg1-Ps85 | Cg69-Pg1-Ps86 | Cg69-Pg1-Ps87 | Cg69-Pg1-Ps88 |
| Cg69-Pg1-Ps89 | Cg69-Pg1-Ps90 | Cg69-Pg1-Ps91 | Cg69-Pg1-Ps92 |
| Cg69-Pg1-Ps93 | Cg69-Pg1-Ps94 | Cg69-Pg1-Ps95 | Cg69-Pg1-Ps96 |
| Cg69-Pg1-Ps97 | Cg69-Pg1-Ps98 | Cg69-Pg1-Ps99 | Cg69-Pg1-Ps100 |
| Cg69-Pg1-Ps101 | Cg69-Pg1-Ps102 | Cg69-Pg1-Ps103 | Cg69-Pg1-Ps104 |
| Cg69-Pg1-Ps105 | Cg69-Pg1-Ps106 | Cg69-Pg1-Ps107 | Cg69-Pg1-Ps108 |
| Cg69-Pg1-Ps109 | Cg69-Pg1-Ps110 | Cg69-Pg1-Ps111 | Cg69-Pg1-Ps112 |
| Cg69-Pg1-Ps113 | Cg69-Pg1-Ps114 | Cg69-Pg1-Ps115 | Cg69-Pg1-Ps116 |
| Cg69-Pg1-Ps117 | Cg69-Pg1-Ps118 | Cg69-Pg1-Ps119 | Cg69-Pg1-Ps120 |
| Cg69-Pg1-Ps121 | Cg69-Pg1-Ps122 | Cg69-Pg1-Ps123 | Cg69-Pg1-Ps124 |
| Cg69-Pg1-Ps125 | Cg69-Pg1-Ps126 | Cg69-Pg1-Ps127 | Cg69-Pg1-Ps128 |
| Cg69-Pg1-Ps129 | Cg69-Pg1-Ps130 | Cg69-Pg1-Ps131 | Cg69-Pg1-Ps132 |
| Cg69-Pg1-Ps133 | Cg69-Pg1-Ps134 | Cg69-Pg1-Ps135 | Cg69-Pg1-Ps136 |
| Cg69-Pg1-Ps137 | Cg69-Pg1-Ps138 | Cg69-Pg1-Ps139 | Cg69-Pg1-Ps140 |
| Cg69-Pg1-Ps141 | Cg69-Pg1-Ps142 | Cg69-Pg1-Ps143 | Cg69-Pg1-Ps144 |
| Cg69-Pg1-Ps145 | Cg69-Pg1-Ps146 | Cg69-Pg1-Ps147 | Cg69-Pg1-Ps148 |
| Cg69-Pg1-Ps149 | Cg69-Pg1-Ps150 | Cg69-Pg1-Ps151 | Cg69-Pg1-Ps152 |
| Cg69-Pg1-Ps153 | Cg69-Pg1-Ps154 | Cg69-Pg1-Ps155 | Cg69-Pg1-Ps156 |
| Cg69-Pg1-Ps157 | Cg69-Pg1-Ps158 | Cg69-Pg1-Ps159 | Cg69-Pg1-Ps160 |
| Cg69-Pg1-Ps161 | Cg69-Pg1-Ps162 | Cg69-Pg1-Ps163 | Cg69-Pg1-Ps164 |
| Cg69-Pg1-Ps165 | Cg69-Pg1-Ps166 | Cg69-Pg1-Ps167 | Cg69-Pg1-Ps168 |
| Cg69-Pg1-Ps169 | Cg69-Pg1-Ps170 | Cg69-Pg1-Ps171 | Cg69-Pg1-Ps172 |
| Cg69-Pg1-Ps173 | Cg69-Pg1-Ps174 | Cg69-Pg1-Ps175 | Cg69-Pg1-Ps176 |
| Cg69-Pg1-Ps177 | Cg69-Pg1-Ps178 | Cg69-Pg1-Ps179 | Cg69-Pg1-Ps180 |
| Cg69-Pg1-Ps181 | Cg69-Pg1-Ps182 | Cg69-Pg1-Ps183 | Cg69-Pg1-Ps184 |
| Cg69-Pg1-Ps185 | Cg69-Pg1-Ps186 | Cg69-Pg1-Ps187 | Cg69-Pg1-Ps188 |
| Cg69-Pg1-Ps189 | Cg69-Pg1-Ps190 | Cg69-Pg1-Ps191 | Cg69-Pg1-Ps192 |
| Cg69-Pg1-Ps193 | Cg69-Pg1-Ps194 | Cg69-Pg1-Ps195 | Cg69-Pg1-Ps196 |
| Cg69-Pg1-Ps197 | Cg69-Pg1-Ps198 | Cg69-Pg1-Ps199 | Cg69-Pg1-Ps200 |
| Cg69-Pg1-Ps201 | Cg69-Pg1-Ps202 | Cg69-Pg1-Ps203 | Cg69-Pg1-Ps204 |
| Cg69-Pg1-Ps205 | Cg69-Pg1-Ps206 | Cg69-Pg1-Ps207 | Cg69-Pg1-Ps208 |
| Cg69-Pg1-Ps209 | Cg69-Pg1-Ps210 | Cg69-Pg1-Ps211 | Cg69-Pg1-Ps212 |
| Cg69-Pg1-Ps213 | Cg69-Pg1-Ps214 | Cg69-Pg1-Ps215 | Cg69-Pg1-Ps216 |
| Cg69-Pg1-Ps217 | Cg69-Pg1-Ps218 | Cg69-Pg1-Ps219 | Cg69-Pg1-Ps220 |
| Cg69-Pg1-Ps221 | Cg69-Pg1-Ps222 | Cg69-Pg1-Ps223 | Cg69-Pg1-Ps224 |
| Cg69-Pg1-Ps225 | Cg69-Pg1-Ps226 | Cg69-Pg1-Ps227 | Cg69-Pg1-Ps228 |
| Cg69-Pg1-Ps229 | Cg69-Pg1-Ps230 | Cg69-Pg1-Ps231 | Cg69-Pg1-Ps232 |
| Cg69-Pg1-Ps233 | Cg69-Pg1-Ps234 | Cg69-Pg1-Ps235 | Cg69-Pg1-Ps236 |
| Cg69-Pg1-Ps237 | Cg69-Pg1-Ps238 | Cg69-Pg1-Ps239 | Cg69-Pg1-Ps240 |
| Cg69-Pg1-Ps241 | Cg69-Pg1-Ps242 | Cg69-Pg1-Ps243 | |
| Cg70-Pg1-Ps1 | Cg70-Pg1-Ps2 | Cg70-Pg1-Ps3 | Cg70-Pg1-Ps4 |
| Cg70-Pg1-Ps5 | Cg70-Pg1-Ps6 | Cg70-Pg1-Ps7 | Cg70-Pg1-Ps8 |
| Cg70-Pg1-Ps9 | Cg70-Pg1-Ps10 | Cg70-Pg1-Ps11 | Cg70-Pg1-Ps12 |
| Cg70-Pg1-Ps13 | Cg70-Pg1-Ps14 | Cg70-Pg1-Ps15 | Cg70-Pg1-Ps16 |
| Cg70-Pg1-Ps17 | Cg70-Pg1-Ps18 | Cg70-Pg1-Ps19 | Cg70-Pg1-Ps20 |
| Cg70-Pg1-Ps21 | Cg70-Pg1-Ps22 | Cg70-Pg1-Ps23 | Cg70-Pg1-Ps24 |
| Cg70-Pg1-Ps25 | Cg70-Pg1-Ps26 | Cg70-Pg1-Ps27 | Cg70-Pg1-Ps28 |
| Cg70-Pg1-Ps29 | Cg70-Pg1-Ps30 | Cg70-Pg1-Ps31 | Cg70-Pg1-Ps32 |
| Cg70-Pg1-Ps33 | Cg70-Pg1-Ps34 | Cg70-Pg1-Ps35 | Cg70-Pg1-Ps36 |
| Cg70-Pg1-Ps37 | Cg70-Pg1-Ps38 | Cg70-Pg1-Ps39 | Cg70-Pg1-Ps40 |
| Cg70-Pg1-Ps41 | Cg70-Pg1-Ps42 | Cg70-Pg1-Ps43 | Cg70-Pg1-Ps44 |
| Cg70-Pg1-Ps45 | Cg70-Pg1-Ps46 | Cg70-Pg1-Ps47 | Cg70-Pg1-Ps48 |
| Cg70-Pg1-Ps49 | Cg70-Pg1-Ps50 | Cg70-Pg1-Ps51 | Cg70-Pg1-Ps52 |
| Cg70-Pg1-Ps53 | Cg70-Pg1-Ps54 | Cg70-Pg1-Ps55 | Cg70-Pg1-Ps56 |
| Cg70-Pg1-Ps57 | Cg70-Pg1-Ps58 | Cg70-Pg1-Ps59 | Cg70-Pg1-Ps60 |
| Cg70-Pg1-Ps61 | Cg70-Pg1-Ps62 | Cg70-Pg1-Ps63 | Cg70-Pg1-Ps64 |
| Cg70-Pg1-Ps65 | Cg70-Pg1-Ps66 | Cg70-Pg1-Ps67 | Cg70-Pg1-Ps68 |
| Cg70-Pg1-Ps69 | Cg70-Pg1-Ps70 | Cg70-Pg1-Ps71 | Cg70-Pg1-Ps72 |
| Cg70-Pg1-Ps73 | Cg70-Pg1-Ps74 | Cg70-Pg1-Ps75 | Cg70-Pg1-Ps76 |
| Cg70-Pg1-Ps77 | Cg70-Pg1-Ps78 | Cg70-Pg1-Ps79 | Cg70-Pg1-Ps80 |
| Cg70-Pg1-Ps81 | Cg70-Pg1-Ps82 | Cg70-Pg1-Ps83 | Cg70-Pg1-Ps84 |
| Cg70-Pg1-Ps85 | Cg70-Pg1-Ps86 | Cg70-Pg1-Ps87 | Cg70-Pg1-Ps88 |
| Cg70-Pg1-Ps89 | Cg70-Pg1-Ps90 | Cg70-Pg1-Ps91 | Cg70-Pg1-Ps92 |
| Cg70-Pg1-Ps93 | Cg70-Pg1-Ps94 | Cg70-Pg1-Ps95 | Cg70-Pg1-Ps96 |
| Cg70-Pg1-Ps97 | Cg70-Pg1-Ps98 | Cg70-Pg1-Ps99 | Cg70-Pg1-Ps100 |
| Cg70-Pg1-Ps101 | Cg70-Pg1-Ps102 | Cg70-Pg1-Ps103 | Cg70-Pg1-Ps104 |
| Cg70-Pg1-Ps105 | Cg70-Pg1-Ps106 | Cg70-Pg1-Ps107 | Cg70-Pg1-Ps108 |
| Cg70-Pg1-Ps109 | Cg70-Pg1-Ps110 | Cg70-Pg1-Ps111 | Cg70-Pg1-Ps112 |
| Cg70-Pg1-Ps113 | Cg70-Pg1-Ps114 | Cg70-Pg1-Ps115 | Cg70-Pg1-Ps116 |
| Cg70-Pg1-Ps117 | Cg70-Pg1-Ps118 | Cg70-Pg1-Ps119 | Cg70-Pg1-Ps120 |
| Cg70-Pg1-Ps121 | Cg70-Pg1-Ps122 | Cg70-Pg1-Ps123 | Cg70-Pg1-Ps124 |
| Cg70-Pg1-Ps125 | Cg70-Pg1-Ps126 | Cg70-Pg1-Ps127 | Cg70-Pg1-Ps128 |
| Cg70-Pg1-Ps129 | Cg70-Pg1-Ps130 | Cg70-Pg1-Ps131 | Cg70-Pg1-Ps132 |
| Cg70-Pg1-Ps133 | Cg70-Pg1-Ps134 | Cg70-Pg1-Ps135 | Cg70-Pg1-Ps136 |
| Cg70-Pg1-Ps137 | Cg70-Pg1-Ps138 | Cg70-Pg1-Ps139 | Cg70-Pg1-Ps140 |
| Cg70-Pg1-Ps141 | Cg70-Pg1-Ps142 | Cg70-Pg1-Ps143 | Cg70-Pg1-Ps144 |
| Cg70-Pg1-Ps145 | Cg70-Pg1-Ps146 | Cg70-Pg1-Ps147 | Cg70-Pg1-Ps148 |
| Cg70-Pg1-Ps149 | Cg70-Pg1-Ps150 | Cg70-Pg1-Ps151 | Cg70-Pg1-Ps152 |
| Cg70-Pg1-Ps153 | Cg70-Pg1-Ps154 | Cg70-Pg1-Ps155 | Cg70-Pg1-Ps156 |
| Cg70-Pg1-Ps157 | Cg70-Pg1-Ps158 | Cg70-Pg1-Ps159 | Cg70-Pg1-Ps160 |
| Cg70-Pg1-Ps161 | Cg70-Pg1-Ps162 | Cg70-Pg1-Ps163 | Cg70-Pg1-Ps164 |
| Cg70-Pg1-Ps165 | Cg70-Pg1-Ps166 | Cg70-Pg1-Ps167 | Cg70-Pg1-Ps168 |
| Cg70-Pg1-Ps169 | Cg70-Pg1-Ps170 | Cg70-Pg1-Ps171 | Cg70-Pg1-Ps172 |
| Cg70-Pg1-Ps173 | Cg70-Pg1-Ps174 | Cg70-Pg1-Ps175 | Cg70-Pg1-Ps176 |
| Cg70-Pg1-Ps177 | Cg70-Pg1-Ps178 | Cg70-Pg1-Ps179 | Cg70-Pg1-Ps180 |
| Cg70-Pg1-Ps181 | Cg70-Pg1-Ps182 | Cg70-Pg1-Ps183 | Cg70-Pg1-Ps184 |
| Cg70-Pg1-Ps185 | Cg70-Pg1-Ps186 | Cg70-Pg1-Ps187 | Cg70-Pg1-Ps188 |
| Cg70-Pg1-Ps189 | Cg70-Pg1-Ps190 | Cg70-Pg1-Ps191 | Cg70-Pg1-Ps192 |
| Cg70-Pg1-Ps193 | Cg70-Pg1-Ps194 | Cg70-Pg1-Ps195 | Cg70-Pg1-Ps196 |
| Cg70-Pg1-Ps197 | Cg70-Pg1-Ps198 | Cg70-Pg1-Ps199 | Cg70-Pg1-Ps200 |
| Cg70-Pg1-Ps201 | Cg70-Pg1-Ps202 | Cg70-Pg1-Ps203 | Cg70-Pg1-Ps204 |
| Cg70-Pg1-Ps205 | Cg70-Pg1-Ps206 | Cg70-Pg1-Ps207 | Cg70-Pg1-Ps208 |
| Cg70-Pg1-Ps209 | Cg70-Pg1-Ps210 | Cg70-Pg1-Ps211 | Cg70-Pg1-Ps212 |
| Cg70-Pg1-Ps213 | Cg70-Pg1-Ps214 | Cg70-Pg1-Ps215 | Cg70-Pg1-Ps216 |
| Cg70-Pg1-Ps217 | Cg70-Pg1-Ps218 | Cg70-Pg1-Ps219 | Cg70-Pg1-Ps220 |
| Cg70-Pg1-Ps221 | Cg70-Pg1-Ps222 | Cg70-Pg1-Ps223 | Cg70-Pg1-Ps224 |
| Cg70-Pg1-Ps225 | Cg70-Pg1-Ps226 | Cg70-Pg1-Ps227 | Cg70-Pg1-Ps228 |
| Cg70-Pg1-Ps229 | Cg70-Pg1-Ps230 | Cg70-Pg1-Ps231 | Cg70-Pg1-Ps232 |
| Cg70-Pg1-Ps233 | Cg70-Pg1-Ps234 | Cg70-Pg1-Ps235 | Cg70-Pg1-Ps236 |
| Cg70-Pg1-Ps237 | Cg70-Pg1-Ps238 | Cg70-Pg1-Ps239 | Cg70-Pg1-Ps240 |
| Cg70-Pg1-Ps241 | Cg70-Pg1-Ps242 | Cg70-Pg1-Ps243 | |
| Cg73-Pg1-Ps1 | Cg73-Pg1-Ps2 | Cg73-Pg1-Ps3 | Cg73-Pg1-Ps4 |
| Cg73-Pg1-Ps5 | Cg73-Pg1-Ps6 | Cg73-Pg1-Ps7 | Cg73-Pg1-Ps8 |
| Cg73-Pg1-Ps9 | Cg73-Pg1-Ps10 | Cg73-Pg1-Ps11 | Cg73-Pg1-Ps12 |
| Cg73-Pg1-Ps13 | Cg73-Pg1-Ps14 | Cg73-Pg1-Ps15 | Cg73-Pg1-Ps16 |
| Cg73-Pg1-Ps17 | Cg73-Pg1-Ps18 | Cg73-Pg1-Ps19 | Cg73-Pg1-Ps20 |
| Cg73-Pg1-Ps21 | Cg73-Pg1-Ps22 | Cg73-Pg1-Ps23 | Cg73-Pg1-Ps24 |
| Cg73-Pg1-Ps25 | Cg73-Pg1-Ps26 | Cg73-Pg1-Ps27 | Cg73-Pg1-Ps28 |
| Cg73-Pg1-Ps29 | Cg73-Pg1-Ps30 | Cg73-Pg1-Ps31 | Cg73-Pg1-Ps32 |
| Cg73-Pg1-Ps33 | Cg73-Pg1-Ps34 | Cg73-Pg1-Ps35 | Cg73-Pg1-Ps36 |
| Cg73-Pg1-Ps37 | Cg73-Pg1-Ps38 | Cg73-Pg1-Ps39 | Cg73-Pg1-Ps40 |
| Cg73-Pg1-Ps41 | Cg73-Pg1-Ps42 | Cg73-Pg1-Ps43 | Cg73-Pg1-Ps44 |
| Cg73-Pg1-Ps45 | Cg73-Pg1-Ps46 | Cg73-Pg1-Ps47 | Cg73-Pg1-Ps48 |
| Cg73-Pg1-Ps49 | Cg73-Pg1-Ps50 | Cg73-Pg1-Ps51 | Cg73-Pg1-Ps52 |
| Cg73-Pg1-Ps53 | Cg73-Pg1-Ps54 | Cg73-Pg1-Ps55 | Cg73-Pg1-Ps56 |
| Cg73-Pg1-Ps57 | Cg73-Pg1-Ps58 | Cg73-Pg1-Ps59 | Cg73-Pg1-Ps60 |
| Cg73-Pg1-Ps61 | Cg73-Pg1-Ps62 | Cg73-Pg1-Ps63 | Cg73-Pg1-Ps64 |
| Cg73-Pg1-Ps65 | Cg73-Pg1-Ps66 | Cg73-Pg1-Ps67 | Cg73-Pg1-Ps68 |
| Cg73-Pg1-Ps69 | Cg73-Pg1-Ps70 | Cg73-Pg1-Ps71 | Cg73-Pg1-Ps72 |
| Cg73-Pg1-Ps73 | Cg73-Pg1-Ps74 | Cg73-Pg1-Ps75 | Cg73-Pg1-Ps76 |
| Cg73-Pg1-Ps77 | Cg73-Pg1-Ps78 | Cg73-Pg1-Ps79 | Cg73-Pg1-Ps80 |
| Cg73-Pg1-Ps81 | Cg73-Pg1-Ps82 | Cg73-Pg1-Ps83 | Cg73-Pg1-Ps84 |
| Cg73-Pg1-Ps85 | Cg73-Pg1-Ps86 | Cg73-Pg1-Ps87 | Cg73-Pg1-Ps88 |
| Cg73-Pg1-Ps89 | Cg73-Pg1-Ps90 | Cg73-Pg1-Ps91 | Cg73-Pg1-Ps92 |
| Cg73-Pg1-Ps93 | Cg73-Pg1-Ps94 | Cg73-Pg1-Ps95 | Cg73-Pg1-Ps96 |
| Cg73-Pg1-Ps97 | Cg73-Pg1-Ps98 | Cg73-Pg1-Ps99 | Cg73-Pg1-Ps100 |
| Cg73-Pg1-Ps101 | Cg73-Pg1-Ps102 | Cg73-Pg1-Ps103 | Cg73-Pg1-Ps104 |
| Cg73-Pg1-Ps105 | Cg73-Pg1-Ps106 | Cg73-Pg1-Ps107 | Cg73-Pg1-Ps108 |
| Cg73-Pg1-Ps109 | Cg73-Pg1-Ps110 | Cg73-Pg1-Ps111 | Cg73-Pg1-Ps112 |
| Cg73-Pg1-Ps113 | Cg73-Pg1-Ps114 | Cg73-Pg1-Ps115 | Cg73-Pg1-Ps116 |
| Cg73-Pg1-Ps117 | Cg73-Pg1-Ps118 | Cg73-Pg1-Ps119 | Cg73-Pg1-Ps120 |
| Cg73-Pg1-Ps121 | Cg73-Pg1-Ps122 | Cg73-Pg1-Ps123 | Cg73-Pg1-Ps124 |
| Cg73-Pg1-Ps125 | Cg73-Pg1-Ps126 | Cg73-Pg1-Ps127 | Cg73-Pg1-Ps128 |
| Cg73-Pg1-Ps129 | Cg73-Pg1-Ps130 | Cg73-Pg1-Ps131 | Cg73-Pg1-Ps132 |
| Cg73-Pg1-Ps133 | Cg73-Pg1-Ps134 | Cg73-Pg1-Ps135 | Cg73-Pg1-Ps136 |
| Cg73-Pg1-Ps137 | Cg73-Pg1-Ps138 | Cg73-Pg1-Ps139 | Cg73-Pg1-Ps140 |
| Cg73-Pg1-Ps141 | Cg73-Pg1-Ps142 | Cg73-Pg1-Ps143 | Cg73-Pg1-Ps144 |
| Cg73-Pg1-Ps145 | Cg73-Pg1-Ps146 | Cg73-Pg1-Ps147 | Cg73-Pg1-Ps148 |
| Cg73-Pg1-Ps149 | Cg73-Pg1-Ps150 | Cg73-Pg1-Ps151 | Cg73-Pg1-Ps152 |
| Cg73-Pg1-Ps153 | Cg73-Pg1-Ps154 | Cg73-Pg1-Ps155 | Cg73-Pg1-Ps156 |
| Cg73-Pg1-Ps157 | Cg73-Pg1-Ps158 | Cg73-Pg1-Ps159 | Cg73-Pg1-Ps160 |
| Cg73-Pg1-Ps161 | Cg73-Pg1-Ps162 | Cg73-Pg1-Ps163 | Cg73-Pg1-Ps164 |
| Cg73-Pg1-Ps165 | Cg73-Pg1-Ps166 | Cg73-Pg1-Ps167 | Cg73-Pg1-Ps168 |
| Cg73-Pg1-Ps169 | Cg73-Pg1-Ps170 | Cg73-Pg1-Ps171 | Cg73-Pg1-Ps172 |
| Cg73-Pg1-Ps173 | Cg73-Pg1-Ps174 | Cg73-Pg1-Ps175 | Cg73-Pg1-Ps176 |
| Cg73-Pg1-Ps177 | Cg73-Pg1-Ps178 | Cg73-Pg1-Ps179 | Cg73-Pg1-Ps180 |
| Cg73-Pg1-Ps181 | Cg73-Pg1-Ps182 | Cg73-Pg1-Ps183 | Cg73-Pg1-Ps184 |
| Cg73-Pg1-Ps185 | Cg73-Pg1-Ps186 | Cg73-Pg1-Ps187 | Cg73-Pg1-Ps188 |
| Cg73-Pg1-Ps189 | Cg73-Pg1-Ps190 | Cg73-Pg1-Ps191 | Cg73-Pg1-Ps192 |
| Cg73-Pg1-Ps193 | Cg73-Pg1-Ps194 | Cg73-Pg1-Ps195 | Cg73-Pg1-Ps196 |
| Cg73-Pg1-Ps197 | Cg73-Pg1-Ps198 | Cg73-Pg1-Ps199 | Cg73-Pg1-Ps200 |
| Cg73-Pg1-Ps201 | Cg73-Pg1-Ps202 | Cg73-Pg1-Ps203 | Cg73-Pg1-Ps204 |
| Cg73-Pg1-Ps205 | Cg73-Pg1-Ps206 | Cg73-Pg1-Ps207 | Cg73-Pg1-Ps208 |
| Cg73-Pg1-Ps209 | Cg73-Pg1-Ps210 | Cg73-Pg1-Ps211 | Cg73-Pg1-Ps212 |
| Cg73-Pg1-Ps213 | Cg73-Pg1-Ps214 | Cg73-Pg1-Ps215 | Cg73-Pg1-Ps216 |
| Cg73-Pg1-Ps217 | Cg73-Pg1-Ps218 | Cg73-Pg1-Ps219 | Cg73-Pg1-Ps220 |
| Cg73-Pg1-Ps221 | Cg73-Pg1-Ps222 | Cg73-Pg1-Ps223 | Cg73-Pg1-Ps224 |
| Cg73-Pg1-Ps225 | Cg73-Pg1-Ps226 | Cg73-Pg1-Ps227 | Cg73-Pg1-Ps228 |
| Cg73-Pg1-Ps229 | Cg73-Pg1-Ps230 | Cg73-Pg1-Ps231 | Cg73-Pg1-Ps232 |
| Cg73-Pg1-Ps233 | Cg73-Pg1-Ps234 | Cg73-Pg1-Ps235 | Cg73-Pg1-Ps236 |
| Cg73-Pg1-Ps237 | Cg73-Pg1-Ps238 | Cg73-Pg1-Ps239 | Cg73-Pg1-Ps240 |
| Cg73-Pg1-Ps241 | Cg73-Pg1-Ps242 | Cg73-Pg1-Ps243 | |
| Cg74-Pg1-Ps1 | Cg74-Pg1-Ps2 | Cg74-Pg1-Ps3 | Cg74-Pg1-Ps4 |
| Cg74-Pg1-Ps5 | Cg74-Pg1-Ps6 | Cg74-Pg1-Ps7 | Cg74-Pg1-Ps8 |
| Cg74-Pg1-Ps9 | Cg74-Pg1-Ps10 | Cg74-Pg1-Ps11 | Cg74-Pg1-Ps12 |
| Cg74-Pg1-Ps13 | Cg74-Pg1-Ps14 | Cg74-Pg1-Ps15 | Cg74-Pg1-Ps16 |
| Cg74-Pg1-Ps17 | Cg74-Pg1-Ps18 | Cg74-Pg1-Ps19 | Cg74-Pg1-Ps20 |
| Cg74-Pg1-Ps21 | Cg74-Pg1-Ps22 | Cg74-Pg1-Ps23 | Cg74-Pg1-Ps24 |
| Cg74-Pg1-Ps25 | Cg74-Pg1-Ps26 | Cg74-Pg1-Ps27 | Cg74-Pg1-Ps28 |
| Cg74-Pg1-Ps29 | Cg74-Pg1-Ps30 | Cg74-Pg1-Ps31 | Cg74-Pg1-Ps32 |
| Cg74-Pg1-Ps33 | Cg74-Pg1-Ps34 | Cg74-Pg1-Ps35 | Cg74-Pg1-Ps36 |
| Cg74-Pg1-Ps37 | Cg74-Pg1-Ps38 | Cg74-Pg1-Ps39 | Cg74-Pg1-Ps40 |
| Cg74-Pg1-Ps41 | Cg74-Pg1-Ps42 | Cg74-Pg1-Ps43 | Cg74-Pg1-Ps44 |
| Cg74-Pg1-Ps45 | Cg74-Pg1-Ps46 | Cg74-Pg1-Ps47 | Cg74-Pg1-Ps48 |
| Cg74-Pg1-Ps49 | Cg74-Pg1-Ps50 | Cg74-Pg1-Ps51 | Cg74-Pg1-Ps52 |
| Cg74-Pg1-Ps53 | Cg74-Pg1-Ps54 | Cg74-Pg1-Ps55 | Cg74-Pg1-Ps56 |
| Cg74-Pg1-Ps57 | Cg74-Pg1-Ps58 | Cg74-Pg1-Ps59 | Cg74-Pg1-Ps60 |
| Cg74-Pg1-Ps61 | Cg74-Pg1-Ps62 | Cg74-Pg1-Ps63 | Cg74-Pg1-Ps64 |
| Cg74-Pg1-Ps65 | Cg74-Pg1-Ps66 | Cg74-Pg1-Ps67 | Cg74-Pg1-Ps68 |
| Cg74-Pg1-Ps69 | Cg74-Pg1-Ps70 | Cg74-Pg1-Ps71 | Cg74-Pg1-Ps72 |
| Cg74-Pg1-Ps73 | Cg74-Pg1-Ps74 | Cg74-Pg1-Ps75 | Cg74-Pg1-Ps76 |
| Cg74-Pg1-Ps77 | Cg74-Pg1-Ps78 | Cg74-Pg1-Ps79 | Cg74-Pg1-Ps80 |
| Cg74-Pg1-Ps81 | Cg74-Pg1-Ps82 | Cg74-Pg1-Ps83 | Cg74-Pg1-Ps84 |
| Cg74-Pg1-Ps85 | Cg74-Pg1-Ps86 | Cg74-Pg1-Ps87 | Cg74-Pg1-Ps88 |
| Cg74-Pg1-Ps89 | Cg74-Pg1-Ps90 | Cg74-Pg1-Ps91 | Cg74-Pg1-Ps92 |
| Cg74-Pg1-Ps93 | Cg74-Pg1-Ps94 | Cg74-Pg1-Ps95 | Cg74-Pg1-Ps96 |
| Cg74-Pg1-Ps97 | Cg74-Pg1-Ps98 | Cg74-Pg1-Ps99 | Cg74-Pg1-Ps100 |
| Cg74-Pg1-Ps101 | Cg74-Pg1-Ps102 | Cg74-Pg1-Ps103 | Cg74-Pg1-Ps104 |
| Cg74-Pg1-Ps105 | Cg74-Pg1-Ps106 | Cg74-Pg1-Ps107 | Cg74-Pg1-Ps108 |
| Cg74-Pg1-Ps109 | Cg74-Pg1-Ps110 | Cg74-Pg1-Ps111 | Cg74-Pg1-Ps112 |
| Cg74-Pg1-Ps113 | Cg74-Pg1-Ps114 | Cg74-Pg1-Ps115 | Cg74-Pg1-Ps116 |
| Cg74-Pg1-Ps117 | Cg74-Pg1-Ps118 | Cg74-Pg1-Ps119 | Cg74-Pg1-Ps120 |
| Cg74-Pg1-Ps121 | Cg74-Pg1-Ps122 | Cg74-Pg1-Ps123 | Cg74-Pg1-Ps124 |
| Cg74-Pg1-Ps125 | Cg74-Pg1-Ps126 | Cg74-Pg1-Ps127 | Cg74-Pg1-Ps128 |
| Cg74-Pg1-Ps129 | Cg74-Pg1-Ps130 | Cg74-Pg1-Ps131 | Cg74-Pg1-Ps132 |
| Cg74-Pg1-Ps133 | Cg74-Pg1-Ps134 | Cg74-Pg1-Ps135 | Cg74-Pg1-Ps136 |
| Cg74-Pg1-Ps137 | Cg74-Pg1-Ps138 | Cg74-Pg1-Ps139 | Cg74-Pg1-Ps140 |
| Cg74-Pg1-Ps141 | Cg74-Pg1-Ps142 | Cg74-Pg1-Ps143 | Cg74-Pg1-Ps144 |
| Cg74-Pg1-Ps145 | Cg74-Pg1-Ps146 | Cg74-Pg1-Ps147 | Cg74-Pg1-Ps148 |
| Cg74-Pg1-Ps149 | Cg74-Pg1-Ps150 | Cg74-Pg1-Ps151 | Cg74-Pg1-Ps152 |
| Cg74-Pg1-Ps153 | Cg74-Pg1-Ps154 | Cg74-Pg1-Ps155 | Cg74-Pg1-Ps156 |
| Cg74-Pg1-Ps157 | Cg74-Pg1-Ps158 | Cg74-Pg1-Ps159 | Cg74-Pg1-Ps160 |
| Cg74-Pg1-Ps161 | Cg74-Pg1-Ps162 | Cg74-Pg1-Ps163 | Cg74-Pg1-Ps164 |
| Cg74-Pg1-Ps165 | Cg74-Pg1-Ps166 | Cg74-Pg1-Ps167 | Cg74-Pg1-Ps168 |
| Cg74-Pg1-Ps169 | Cg74-Pg1-Ps170 | Cg74-Pg1-Ps171 | Cg74-Pg1-Ps172 |
| Cg74-Pg1-Ps173 | Cg74-Pg1-Ps174 | Cg74-Pg1-Ps175 | Cg74-Pg1-Ps176 |
| Cg74-Pg1-Ps177 | Cg74-Pg1-Ps178 | Cg74-Pg1-Ps179 | Cg74-Pg1-Ps180 |
| Cg74-Pg1-Ps181 | Cg74-Pg1-Ps182 | Cg74-Pg1-Ps183 | Cg74-Pg1-Ps184 |
| Cg74-Pg1-Ps185 | Cg74-Pg1-Ps186 | Cg74-Pg1-Ps187 | Cg74-Pg1-Ps188 |
| Cg74-Pg1-Ps189 | Cg74-Pg1-Ps190 | Cg74-Pg1-Ps191 | Cg74-Pg1-Ps192 |
| Cg74-Pg1-Ps193 | Cg74-Pg1-Ps194 | Cg74-Pg1-Ps195 | Cg74-Pg1-Ps196 |
| Cg74-Pg1-Ps197 | Cg74-Pg1-Ps198 | Cg74-Pg1-Ps199 | Cg74-Pg1-Ps200 |
| Cg74-Pg1-Ps201 | Cg74-Pg1-Ps202 | Cg74-Pg1-Ps203 | Cg74-Pg1-Ps204 |
| Cg74-Pg1-Ps205 | Cg74-Pg1-Ps206 | Cg74-Pg1-Ps207 | Cg74-Pg1-Ps208 |
| Cg74-Pg1-Ps209 | Cg74-Pg1-Ps210 | Cg74-Pg1-Ps211 | Cg74-Pg1-Ps212 |
| Cg74-Pg1-Ps213 | Cg74-Pg1-Ps214 | Cg74-Pg1-Ps215 | Cg74-Pg1-Ps216 |
| Cg74-Pg1-Ps217 | Cg74-Pg1-Ps218 | Cg74-Pg1-Ps219 | Cg74-Pg1-Ps220 |
| Cg74-Pg1-Ps221 | Cg74-Pg1-Ps222 | Cg74-Pg1-Ps223 | Cg74-Pg1-Ps224 |
| Cg74-Pg1-Ps225 | Cg74-Pg1-Ps226 | Cg74-Pg1-Ps227 | Cg74-Pg1-Ps228 |
| Cg74-Pg1-Ps229 | Cg74-Pg1-Ps230 | Cg74-Pg1-Ps231 | Cg74-Pg1-Ps232 |
| Cg74-Pg1-Ps233 | Cg74-Pg1-Ps234 | Cg74-Pg1-Ps235 | Cg74-Pg1-Ps236 |
| Cg74-Pg1-Ps237 | Cg74-Pg1-Ps238 | Cg74-Pg1-Ps239 | Cg74-Pg1-Ps240 |
| Cg74-Pg1-Ps241 | Cg74-Pg1-Ps242 | Cg74-Pg1-Ps243 | |
| Cg75-Pg1-Ps1 | Cg75-Pg1-Ps2 | Cg75-Pg1-Ps3 | Cg75-Pg1-Ps4 |
| Cg75-Pg1-Ps5 | Cg75-Pg1-Ps6 | Cg75-Pg1-Ps7 | Cg75-Pg1-Ps8 |
| Cg75-Pg1-Ps9 | Cg75-Pg1-Ps10 | Cg75-Pg1-Ps11 | Cg75-Pg1-Ps12 |
| Cg75-Pg1-Ps13 | Cg75-Pg1-Ps14 | Cg75-Pg1-Ps15 | Cg75-Pg1-Ps16 |
| Cg75-Pg1-Ps17 | Cg75-Pg1-Ps18 | Cg75-Pg1-Ps19 | Cg75-Pg1-Ps20 |
| Cg75-Pg1-Ps21 | Cg75-Pg1-Ps22 | Cg75-Pg1-Ps23 | Cg75-Pg1-Ps24 |
| Cg75-Pg1-Ps25 | Cg75-Pg1-Ps26 | Cg75-Pg1-Ps27 | Cg75-Pg1-Ps28 |
| Cg75-Pg1-Ps29 | Cg75-Pg1-Ps30 | Cg75-Pg1-Ps31 | Cg75-Pg1-Ps32 |
| Cg75-Pg1-Ps33 | Cg75-Pg1-Ps34 | Cg75-Pg1-Ps35 | Cg75-Pg1-Ps36 |
| Cg75-Pg1-Ps37 | Cg75-Pg1-Ps38 | Cg75-Pg1-Ps39 | Cg75-Pg1-Ps40 |
| Cg75-Pg1-Ps41 | Cg75-Pg1-Ps42 | Cg75-Pg1-Ps43 | Cg75-Pg1-Ps44 |
| Cg75-Pg1-Ps45 | Cg75-Pg1-Ps46 | Cg75-Pg1-Ps47 | Cg75-Pg1-Ps48 |
| Cg75-Pg1-Ps49 | Cg75-Pg1-Ps50 | Cg75-Pg1-Ps51 | Cg75-Pg1-Ps52 |
| Cg75-Pg1-Ps53 | Cg75-Pg1-Ps54 | Cg75-Pg1-Ps55 | Cg75-Pg1-Ps56 |
| Cg75-Pg1-Ps57 | Cg75-Pg1-Ps58 | Cg75-Pg1-Ps59 | Cg75-Pg1-Ps60 |
| Cg75-Pg1-Ps61 | Cg75-Pg1-Ps62 | Cg75-Pg1-Ps63 | Cg75-Pg1-Ps64 |
| Cg75-Pg1-Ps65 | Cg75-Pg1-Ps66 | Cg75-Pg1-Ps67 | Cg75-Pg1-Ps68 |
| Cg75-Pg1-Ps69 | Cg75-Pg1-Ps70 | Cg75-Pg1-Ps71 | Cg75-Pg1-Ps72 |
| Cg75-Pg1-Ps73 | Cg75-Pg1-Ps74 | Cg75-Pg1-Ps75 | Cg75-Pg1-Ps76 |
| Cg75-Pg1-Ps77 | Cg75-Pg1-Ps78 | Cg75-Pg1-Ps79 | Cg75-Pg1-Ps80 |
| Cg75-Pg1-Ps81 | Cg75-Pg1-Ps82 | Cg75-Pg1-Ps83 | Cg75-Pg1-Ps84 |
| Cg75-Pg1-Ps85 | Cg75-Pg1-Ps86 | Cg75-Pg1-Ps87 | Cg75-Pg1-Ps88 |
| Cg75-Pg1-Ps89 | Cg75-Pg1-Ps90 | Cg75-Pg1-Ps91 | Cg75-Pg1-Ps92 |
| Cg75-Pg1-Ps93 | Cg75-Pg1-Ps94 | Cg75-Pg1-Ps95 | Cg75-Pg1-Ps96 |
| Cg75-Pg1-Ps97 | Cg75-Pg1-Ps98 | Cg75-Pg1-Ps99 | Cg75-Pg1-Ps100 |
| Cg75-Pg1-Ps101 | Cg75-Pg1-Ps102 | Cg75-Pg1-Ps103 | Cg75-Pg1-Ps104 |
| Cg75-Pg1-Ps105 | Cg75-Pg1-Ps106 | Cg75-Pg1-Ps107 | Cg75-Pg1-Ps108 |
| Cg75-Pg1-Ps109 | Cg75-Pg1-Ps110 | Cg75-Pg1-Ps111 | Cg75-Pg1-Ps112 |
| Cg75-Pg1-Ps113 | Cg75-Pg1-Ps114 | Cg75-Pg1-Ps115 | Cg75-Pg1-Ps116 |
| Cg75-Pg1-Ps117 | Cg75-Pg1-Ps118 | Cg75-Pg1-Ps119 | Cg75-Pg1-Ps120 |
| Cg75-Pg1-Ps121 | Cg75-Pg1-Ps122 | Cg75-Pg1-Ps123 | Cg75-Pg1-Ps124 |
| Cg75-Pg1-Ps125 | Cg75-Pg1-Ps126 | Cg75-Pg1-Ps127 | Cg75-Pg1-Ps128 |
| Cg75-Pg1-Ps129 | Cg75-Pg1-Ps130 | Cg75-Pg1-Ps131 | Cg75-Pg1-Ps132 |
| Cg75-Pg1-Ps133 | Cg75-Pg1-Ps134 | Cg75-Pg1-Ps135 | Cg75-Pg1-Ps136 |
| Cg75-Pg1-Ps137 | Cg75-Pg1-Ps138 | Cg75-Pg1-Ps139 | Cg75-Pg1-Ps140 |
| Cg75-Pg1-Ps141 | Cg75-Pg1-Ps142 | Cg75-Pg1-Ps143 | Cg75-Pg1-Ps144 |
| Cg75-Pg1-Ps145 | Cg75-Pg1-Ps146 | Cg75-Pg1-Ps147 | Cg75-Pg1-Ps148 |
| Cg75-Pg1-Ps149 | Cg75-Pg1-Ps150 | Cg75-Pg1-Ps151 | Cg75-Pg1-Ps152 |
| Cg75-Pg1-Ps153 | Cg75-Pg1-Ps154 | Cg75-Pg1-Ps155 | Cg75-Pg1-Ps156 |
| Cg75-Pg1-Ps157 | Cg75-Pg1-Ps158 | Cg75-Pg1-Ps159 | Cg75-Pg1-Ps160 |
| Cg75-Pg1-Ps161 | Cg75-Pg1-Ps162 | Cg75-Pg1-Ps163 | Cg75-Pg1-Ps164 |
| Cg75-Pg1-Ps165 | Cg75-Pg1-Ps166 | Cg75-Pg1-Ps167 | Cg75-Pg1-Ps168 |
| Cg75-Pg1-Ps169 | Cg75-Pg1-Ps170 | Cg75-Pg1-Ps171 | Cg75-Pg1-Ps172 |
| Cg75-Pg1-Ps173 | Cg75-Pg1-Ps174 | Cg75-Pg1-Ps175 | Cg75-Pg1-Ps176 |
| Cg75-Pg1-Ps177 | Cg75-Pg1-Ps178 | Cg75-Pg1-Ps179 | Cg75-Pg1-Ps180 |
| Cg75-Pg1-Ps181 | Cg75-Pg1-Ps182 | Cg75-Pg1-Ps183 | Cg75-Pg1-Ps184 |
| Cg75-Pg1-Ps185 | Cg75-Pg1-Ps186 | Cg75-Pg1-Ps187 | Cg75-Pg1-Ps188 |
| Cg75-Pg1-Ps189 | Cg75-Pg1-Ps190 | Cg75-Pg1-Ps191 | Cg75-Pg1-Ps192 |
| Cg75-Pg1-Ps193 | Cg75-Pg1-Ps194 | Cg75-Pg1-Ps195 | Cg75-Pg1-Ps196 |
| Cg75-Pg1-Ps197 | Cg75-Pg1-Ps198 | Cg75-Pg1-Ps199 | Cg75-Pg1-Ps200 |
| Cg75-Pg1-Ps201 | Cg75-Pg1-Ps202 | Cg75-Pg1-Ps203 | Cg75-Pg1-Ps204 |
| Cg75-Pg1-Ps205 | Cg75-Pg1-Ps206 | Cg75-Pg1-Ps207 | Cg75-Pg1-Ps208 |
| Cg75-Pg1-Ps209 | Cg75-Pg1-Ps210 | Cg75-Pg1-Ps211 | Cg75-Pg1-Ps212 |
| Cg75-Pg1-Ps213 | Cg75-Pg1-Ps214 | Cg75-Pg1-Ps215 | Cg75-Pg1-Ps216 |
| Cg75-Pg1-Ps217 | Cg75-Pg1-Ps218 | Cg75-Pg1-Ps219 | Cg75-Pg1-Ps220 |
| Cg75-Pg1-Ps221 | Cg75-Pg1-Ps222 | Cg75-Pg1-Ps223 | Cg75-Pg1-Ps224 |
| Cg75-Pg1-Ps225 | Cg75-Pg1-Ps226 | Cg75-Pg1-Ps227 | Cg75-Pg1-Ps228 |
| Cg75-Pg1-Ps229 | Cg75-Pg1-Ps230 | Cg75-Pg1-Ps231 | Cg75-Pg1-Ps232 |
| Cg75-Pg1-Ps233 | Cg75-Pg1-Ps234 | Cg75-Pg1-Ps235 | Cg75-Pg1-Ps236 |
| Cg75-Pg1-Ps237 | Cg75-Pg1-Ps238 | Cg75-Pg1-Ps239 | Cg75-Pg1-Ps240 |
| Cg75-Pg1-Ps241 | Cg75-Pg1-Ps242 | Cg75-Pg1-Ps243 | |
| Cg76-Pg1-Ps1 | Cg76-Pg1-Ps2 | Cg76-Pg1-Ps3 | Cg76-Pg1-Ps4 |
| Cg76-Pg1-Ps5 | Cg76-Pg1-Ps6 | Cg76-Pg1-Ps7 | Cg76-Pg1-Ps8 |
| Cg76-Pg1-Ps9 | Cg76-Pg1-Ps10 | Cg76-Pg1-Ps11 | Cg76-Pg1-Ps12 |
| Cg76-Pg1-Ps13 | Cg76-Pg1-Ps14 | Cg76-Pg1-Ps15 | Cg76-Pg1-Ps16 |
| Cg76-Pg1-Ps17 | Cg76-Pg1-Ps18 | Cg76-Pg1-Ps19 | Cg76-Pg1-Ps20 |
| Cg76-Pg1-Ps21 | Cg76-Pg1-Ps22 | Cg76-Pg1-Ps23 | Cg76-Pg1-Ps24 |
| Cg76-Pg1-Ps25 | Cg76-Pg1-Ps26 | Cg76-Pg1-Ps27 | Cg76-Pg1-Ps28 |
| Cg76-Pg1-Ps29 | Cg76-Pg1-Ps30 | Cg76-Pg1-Ps31 | Cg76-Pg1-Ps32 |
| Cg76-Pg1-Ps33 | Cg76-Pg1-Ps34 | Cg76-Pg1-Ps35 | Cg76-Pg1-Ps36 |
| Cg76-Pg1-Ps37 | Cg76-Pg1-Ps38 | Cg76-Pg1-Ps39 | Cg76-Pg1-Ps40 |
| Cg76-Pg1-Ps41 | Cg76-Pg1-Ps42 | Cg76-Pg1-Ps43 | Cg76-Pg1-Ps44 |
| Cg76-Pg1-Ps45 | Cg76-Pg1-Ps46 | Cg76-Pg1-Ps47 | Cg76-Pg1-Ps48 |
| Cg76-Pg1-Ps49 | Cg76-Pg1-Ps50 | Cg76-Pg1-Ps51 | Cg76-Pg1-Ps52 |
| Cg76-Pg1-Ps53 | Cg76-Pg1-Ps54 | Cg76-Pg1-Ps55 | Cg76-Pg1-Ps56 |
| Cg76-Pg1-Ps57 | Cg76-Pg1-Ps58 | Cg76-Pg1-Ps59 | Cg76-Pg1-Ps60 |
| Cg76-Pg1-Ps61 | Cg76-Pg1-Ps62 | Cg76-Pg1-Ps63 | Cg76-Pg1-Ps64 |
| Cg76-Pg1-Ps65 | Cg76-Pg1-Ps66 | Cg76-Pg1-Ps67 | Cg76-Pg1-Ps68 |
| Cg76-Pg1-Ps69 | Cg76-Pg1-Ps70 | Cg76-Pg1-Ps71 | Cg76-Pg1-Ps72 |
| Cg76-Pg1-Ps73 | Cg76-Pg1-Ps74 | Cg76-Pg1-Ps75 | Cg76-Pg1-Ps76 |
| Cg76-Pg1-Ps77 | Cg76-Pg1-Ps78 | Cg76-Pg1-Ps79 | Cg76-Pg1-Ps80 |
| Cg76-Pg1-Ps81 | Cg76-Pg1-Ps82 | Cg76-Pg1-Ps83 | Cg76-Pg1-Ps84 |
| Cg76-Pg1-Ps85 | Cg76-Pg1-Ps86 | Cg76-Pg1-Ps87 | Cg76-Pg1-Ps88 |
| Cg76-Pg1-Ps89 | Cg76-Pg1-Ps90 | Cg76-Pg1-Ps91 | Cg76-Pg1-Ps92 |
| Cg76-Pg1-Ps93 | Cg76-Pg1-Ps94 | Cg76-Pg1-Ps95 | Cg76-Pg1-Ps96 |
| Cg76-Pg1-Ps97 | Cg76-Pg1-Ps98 | Cg76-Pg1-Ps99 | Cg76-Pg1-Ps100 |
| Cg76-Pg1-Ps101 | Cg76-Pg1-Ps102 | Cg76-Pg1-Ps103 | Cg76-Pg1-Ps104 |
| Cg76-Pg1-Ps105 | Cg76-Pg1-Ps106 | Cg76-Pg1-Ps107 | Cg76-Pg1-Ps108 |
| Cg76-Pg1-Ps109 | Cg76-Pg1-Ps110 | Cg76-Pg1-Ps111 | Cg76-Pg1-Ps112 |
| Cg76-Pg1-Ps113 | Cg76-Pg1-Ps114 | Cg76-Pg1-Ps115 | Cg76-Pg1-Ps116 |
| Cg76-Pg1-Ps117 | Cg76-Pg1-Ps118 | Cg76-Pg1-Ps119 | Cg76-Pg1-Ps120 |
| Cg76-Pg1-Ps121 | Cg76-Pg1-Ps122 | Cg76-Pg1-Ps123 | Cg76-Pg1-Ps124 |
| Cg76-Pg1-Ps125 | Cg76-Pg1-Ps126 | Cg76-Pg1-Ps127 | Cg76-Pg1-Ps128 |
| Cg76-Pg1-Ps129 | Cg76-Pg1-Ps130 | Cg76-Pg1-Ps131 | Cg76-Pg1-Ps132 |
| Cg76-Pg1-Ps133 | Cg76-Pg1-Ps134 | Cg76-Pg1-Ps135 | Cg76-Pg1-Ps136 |
| Cg76-Pg1-Ps137 | Cg76-Pg1-Ps138 | Cg76-Pg1-Ps139 | Cg76-Pg1-Ps140 |
| Cg76-Pg1-Ps141 | Cg76-Pg1-Ps142 | Cg76-Pg1-Ps143 | Cg76-Pg1-Ps144 |
| Cg76-Pg1-Ps145 | Cg76-Pg1-Ps146 | Cg76-Pg1-Ps147 | Cg76-Pg1-Ps148 |
| Cg76-Pg1-Ps149 | Cg76-Pg1-Ps150 | Cg76-Pg1-Ps151 | Cg76-Pg1-Ps152 |
| Cg76-Pg1-Ps153 | Cg76-Pg1-Ps154 | Cg76-Pg1-Ps155 | Cg76-Pg1-Ps156 |
| Cg76-Pg1-Ps157 | Cg76-Pg1-Ps158 | Cg76-Pg1-Ps159 | Cg76-Pg1-Ps160 |
| Cg76-Pg1-Ps161 | Cg76-Pg1-Ps162 | Cg76-Pg1-Ps163 | Cg76-Pg1-Ps164 |
| Cg76-Pg1-Ps165 | Cg76-Pg1-Ps166 | Cg76-Pg1-Ps167 | Cg76-Pg1-Ps168 |
| Cg76-Pg1-Ps169 | Cg76-Pg1-Ps170 | Cg76-Pg1-Ps171 | Cg76-Pg1-Ps172 |
| Cg76-Pg1-Ps173 | Cg76-Pg1-Ps174 | Cg76-Pg1-Ps175 | Cg76-Pg1-Ps176 |
| Cg76-Pg1-Ps177 | Cg76-Pg1-Ps178 | Cg76-Pg1-Ps179 | Cg76-Pg1-Ps180 |
| Cg76-Pg1-Ps181 | Cg76-Pg1-Ps182 | Cg76-Pg1-Ps183 | Cg76-Pg1-Ps184 |
| Cg76-Pg1-Ps185 | Cg76-Pg1-Ps186 | Cg76-Pg1-Ps187 | Cg76-Pg1-Ps188 |
| Cg76-Pg1-Ps189 | Cg76-Pg1-Ps190 | Cg76-Pg1-Ps191 | Cg76-Pg1-Ps192 |
| Cg76-Pg1-Ps193 | Cg76-Pg1-Ps194 | Cg76-Pg1-Ps195 | Cg76-Pg1-Ps196 |
| Cg76-Pg1-Ps197 | Cg76-Pg1-Ps198 | Cg76-Pg1-Ps199 | Cg76-Pg1-Ps200 |
| Cg76-Pg1-Ps201 | Cg76-Pg1-Ps202 | Cg76-Pg1-Ps203 | Cg76-Pg1-Ps204 |
| Cg76-Pg1-Ps205 | Cg76-Pg1-Ps206 | Cg76-Pg1-Ps207 | Cg76-Pg1-Ps208 |
| Cg76-Pg1-Ps209 | Cg76-Pg1-Ps210 | Cg76-Pg1-Ps211 | Cg76-Pg1-Ps212 |
| Cg76-Pg1-Ps213 | Cg76-Pg1-Ps214 | Cg76-Pg1-Ps215 | Cg76-Pg1-Ps216 |
| Cg76-Pg1-Ps217 | Cg76-Pg1-Ps218 | Cg76-Pg1-Ps219 | Cg76-Pg1-Ps220 |
| Cg76-Pg1-Ps221 | Cg76-Pg1-Ps222 | Cg76-Pg1-Ps223 | Cg76-Pg1-Ps224 |
| Cg76-Pg1-Ps225 | Cg76-Pg1-Ps226 | Cg76-Pg1-Ps227 | Cg76-Pg1-Ps228 |
| Cg76-Pg1-Ps229 | Cg76-Pg1-Ps230 | Cg76-Pg1-Ps231 | Cg76-Pg1-Ps232 |
| Cg76-Pg1-Ps233 | Cg76-Pg1-Ps234 | Cg76-Pg1-Ps235 | Cg76-Pg1-Ps236 |
| Cg76-Pg1-Ps237 | Cg76-Pg1-Ps238 | Cg76-Pg1-Ps239 | Cg76-Pg1-Ps240 |
| Cg76-Pg1-Ps241 | Cg76-Pg1-Ps242 | Cg76-Pg1-Ps243 | |
| Cg81-Pg1-Ps1 | Cg81-Pg1-Ps2 | Cg81-Pg1-Ps3 | Cg81-Pg1-Ps4 |
| Cg81-Pg1-Ps5 | Cg81-Pg1-Ps6 | Cg81-Pg1-Ps7 | Cg81-Pg1-Ps8 |
| Cg81-Pg1-Ps9 | Cg81-Pg1-Ps10 | Cg81-Pg1-Ps11 | Cg81-Pg1-Ps12 |
| Cg81-Pg1-Ps13 | Cg81-Pg1-Ps14 | Cg81-Pg1-Ps15 | Cg81-Pg1-Ps16 |
| Cg81-Pg1-Ps17 | Cg81-Pg1-Ps18 | Cg81-Pg1-Ps19 | Cg81-Pg1-Ps20 |
| Cg81-Pg1-Ps21 | Cg81-Pg1-Ps22 | Cg81-Pg1-Ps23 | Cg81-Pg1-Ps24 |
| Cg81-Pg1-Ps25 | Cg81-Pg1-Ps26 | Cg81-Pg1-Ps27 | Cg81-Pg1-Ps28 |
| Cg81-Pg1-Ps29 | Cg81-Pg1-Ps30 | Cg81-Pg1-Ps31 | Cg81-Pg1-Ps32 |
| Cg81-Pg1-Ps33 | Cg81-Pg1-Ps34 | Cg81-Pg1-Ps35 | Cg81-Pg1-Ps36 |
| Cg81-Pg1-Ps37 | Cg81-Pg1-Ps38 | Cg81-Pg1-Ps39 | Cg81-Pg1-Ps40 |
| Cg81-Pg1-Ps41 | Cg81-Pg1-Ps42 | Cg81-Pg1-Ps43 | Cg81-Pg1-Ps44 |
| Cg81-Pg1-Ps45 | Cg81-Pg1-Ps46 | Cg81-Pg1-Ps47 | Cg81-Pg1-Ps48 |
| Cg81-Pg1-Ps49 | Cg81-Pg1-Ps50 | Cg81-Pg1-Ps51 | Cg81-Pg1-Ps52 |
| Cg81-Pg1-Ps53 | Cg81-Pg1-Ps54 | Cg81-Pg1-Ps55 | Cg81-Pg1-Ps56 |
| Cg81-Pg1-Ps57 | Cg81-Pg1-Ps58 | Cg81-Pg1-Ps59 | Cg81-Pg1-Ps60 |
| Cg81-Pg1-Ps61 | Cg81-Pg1-Ps62 | Cg81-Pg1-Ps63 | Cg81-Pg1-Ps64 |
| Cg81-Pg1-Ps65 | Cg81-Pg1-Ps66 | Cg81-Pg1-Ps67 | Cg81-Pg1-Ps68 |
| Cg81-Pg1-Ps69 | Cg81-Pg1-Ps70 | Cg81-Pg1-Ps71 | Cg81-Pg1-Ps72 |
| Cg81-Pg1-Ps73 | Cg81-Pg1-Ps74 | Cg81-Pg1-Ps75 | Cg81-Pg1-Ps76 |
| Cg81-Pg1-Ps77 | Cg81-Pg1-Ps78 | Cg81-Pg1-Ps79 | Cg81-Pg1-Ps80 |
| Cg81-Pg1-Ps81 | Cg81-Pg1-Ps82 | Cg81-Pg1-Ps83 | Cg81-Pg1-Ps84 |
| Cg81-Pg1-Ps85 | Cg81-Pg1-Ps86 | Cg81-Pg1-Ps87 | Cg81-Pg1-Ps88 |
| Cg81-Pg1-Ps89 | Cg81-Pg1-Ps90 | Cg81-Pg1-Ps91 | Cg81-Pg1-Ps92 |
| Cg81-Pg1-Ps93 | Cg81-Pg1-Ps94 | Cg81-Pg1-Ps95 | Cg81-Pg1-Ps96 |
| Cg81-Pg1-Ps97 | Cg81-Pg1-Ps98 | Cg81-Pg1-Ps99 | Cg81-Pg1-Ps100 |
| Cg81-Pg1-Ps101 | Cg81-Pg1-Ps102 | Cg81-Pg1-Ps103 | Cg81-Pg1-Ps104 |
| Cg81-Pg1-Ps105 | Cg81-Pg1-Ps106 | Cg81-Pg1-Ps107 | Cg81-Pg1-Ps108 |
| Cg81-Pg1-Ps109 | Cg81-Pg1-Ps110 | Cg81-Pg1-Ps111 | Cg81-Pg1-Ps112 |
| Cg81-Pg1-Ps113 | Cg81-Pg1-Ps114 | Cg81-Pg1-Ps115 | Cg81-Pg1-Ps116 |
| Cg81-Pg1-Ps117 | Cg81-Pg1-Ps118 | Cg81-Pg1-Ps119 | Cg81-Pg1-Ps120 |
| Cg81-Pg1-Ps121 | Cg81-Pg1-Ps122 | Cg81-Pg1-Ps123 | Cg81-Pg1-Ps124 |
| Cg81-Pg1-Ps125 | Cg81-Pg1-Ps126 | Cg81-Pg1-Ps127 | Cg81-Pg1-Ps128 |
| Cg81-Pg1-Ps129 | Cg81-Pg1-Ps130 | Cg81-Pg1-Ps131 | Cg81-Pg1-Ps132 |
| Cg81-Pg1-Ps133 | Cg81-Pg1-Ps134 | Cg81-Pg1-Ps135 | Cg81-Pg1-Ps136 |
| Cg81-Pg1-Ps137 | Cg81-Pg1-Ps138 | Cg81-Pg1-Ps139 | Cg81-Pg1-Ps140 |
| Cg81-Pg1-Ps141 | Cg81-Pg1-Ps142 | Cg81-Pg1-Ps143 | Cg81-Pg1-Ps144 |
| Cg81-Pg1-Ps145 | Cg81-Pg1-Ps146 | Cg81-Pg1-Ps147 | Cg81-Pg1-Ps148 |
| Cg81-Pg1-Ps149 | Cg81-Pg1-Ps150 | Cg81-Pg1-Ps151 | Cg81-Pg1-Ps152 |
| Cg81-Pg1-Ps153 | Cg81-Pg1-Ps154 | Cg81-Pg1-Ps155 | Cg81-Pg1-Ps156 |
| Cg81-Pg1-Ps157 | Cg81-Pg1-Ps158 | Cg81-Pg1-Ps159 | Cg81-Pg1-Ps160 |
| Cg81-Pg1-Ps161 | Cg81-Pg1-Ps162 | Cg81-Pg1-Ps163 | Cg81-Pg1-Ps164 |
| Cg81-Pg1-Ps165 | Cg81-Pg1-Ps166 | Cg81-Pg1-Ps167 | Cg81-Pg1-Ps168 |
| Cg81-Pg1-Ps169 | Cg81-Pg1-Ps170 | Cg81-Pg1-Ps171 | Cg81-Pg1-Ps172 |
| Cg81-Pg1-Ps173 | Cg81-Pg1-Ps174 | Cg81-Pg1-Ps175 | Cg81-Pg1-Ps176 |
| Cg81-Pg1-Ps177 | Cg81-Pg1-Ps178 | Cg81-Pg1-Ps179 | Cg81-Pg1-Ps180 |
| Cg81-Pg1-Ps181 | Cg81-Pg1-Ps182 | Cg81-Pg1-Ps183 | Cg81-Pg1-Ps184 |
| Cg81-Pg1-Ps185 | Cg81-Pg1-Ps186 | Cg81-Pg1-Ps187 | Cg81-Pg1-Ps188 |
| Cg81-Pg1-Ps189 | Cg81-Pg1-Ps190 | Cg81-Pg1-Ps191 | Cg81-Pg1-Ps192 |
| Cg81-Pg1-Ps193 | Cg81-Pg1-Ps194 | Cg81-Pg1-Ps195 | Cg81-Pg1-Ps196 |
| Cg81-Pg1-Ps197 | Cg81-Pg1-Ps198 | Cg81-Pg1-Ps199 | Cg81-Pg1-Ps200 |
| Cg81-Pg1-Ps201 | Cg81-Pg1-Ps202 | Cg81-Pg1-Ps203 | Cg81-Pg1-Ps204 |
| Cg81-Pg1-Ps205 | Cg81-Pg1-Ps206 | Cg81-Pg1-Ps207 | Cg81-Pg1-Ps208 |
| Cg81-Pg1-Ps209 | Cg81-Pg1-Ps210 | Cg81-Pg1-Ps211 | Cg81-Pg1-Ps212 |
| Cg81-Pg1-Ps213 | Cg81-Pg1-Ps214 | Cg81-Pg1-Ps215 | Cg81-Pg1-Ps216 |
| Cg81-Pg1-Ps217 | Cg81-Pg1-Ps218 | Cg81-Pg1-Ps219 | Cg81-Pg1-Ps220 |
| Cg81-Pg1-Ps221 | Cg81-Pg1-Ps222 | Cg81-Pg1-Ps223 | Cg81-Pg1-Ps224 |
| Cg81-Pg1-Ps225 | Cg81-Pg1-Ps226 | Cg81-Pg1-Ps227 | Cg81-Pg1-Ps228 |
| Cg81-Pg1-Ps229 | Cg81-Pg1-Ps230 | Cg81-Pg1-Ps231 | Cg81-Pg1-Ps232 |
| Cg81-Pg1-Ps233 | Cg81-Pg1-Ps234 | Cg81-Pg1-Ps235 | Cg81-Pg1-Ps236 |
| Cg81-Pg1-Ps237 | Cg81-Pg1-Ps238 | Cg81-Pg1-Ps239 | Cg81-Pg1-Ps240 |
| Cg81-Pg1-Ps241 | Cg81-Pg1-Ps242 | Cg81-Pg1-Ps243 | |
| Cg82-Pg1-Ps1 | Cg82-Pg1-Ps2 | Cg82-Pg1-Ps3 | Cg82-Pg1-Ps4 |
| Cg82-Pg1-Ps5 | Cg82-Pg1-Ps6 | Cg82-Pg1-Ps7 | Cg82-Pg1-Ps8 |
| Cg82-Pg1-Ps9 | Cg82-Pg1-Ps10 | Cg82-Pg1-Ps11 | Cg82-Pg1-Ps12 |
| Cg82-Pg1-Ps13 | Cg82-Pg1-Ps14 | Cg82-Pg1-Ps15 | Cg82-Pg1-Ps16 |
| Cg82-Pg1-Ps17 | Cg82-Pg1-Ps18 | Cg82-Pg1-Ps19 | Cg82-Pg1-Ps20 |
| Cg82-Pg1-Ps21 | Cg82-Pg1-Ps22 | Cg82-Pg1-Ps23 | Cg82-Pg1-Ps24 |
| Cg82-Pg1-Ps25 | Cg82-Pg1-Ps26 | Cg82-Pg1-Ps27 | Cg82-Pg1-Ps28 |
| Cg82-Pg1-Ps29 | Cg82-Pg1-Ps30 | Cg82-Pg1-Ps31 | Cg82-Pg1-Ps32 |
| Cg82-Pg1-Ps33 | Cg82-Pg1-Ps34 | Cg82-Pg1-Ps35 | Cg82-Pg1-Ps36 |
| Cg82-Pg1-Ps37 | Cg82-Pg1-Ps38 | Cg82-Pg1-Ps39 | Cg82-Pg1-Ps40 |
| Cg82-Pg1-Ps41 | Cg82-Pg1-Ps42 | Cg82-Pg1-Ps43 | Cg82-Pg1-Ps44 |
| Cg82-Pg1-Ps45 | Cg82-Pg1-Ps46 | Cg82-Pg1-Ps47 | Cg82-Pg1-Ps48 |
| Cg82-Pg1-Ps49 | Cg82-Pg1-Ps50 | Cg82-Pg1-Ps51 | Cg82-Pg1-Ps52 |
| Cg82-Pg1-Ps53 | Cg82-Pg1-Ps54 | Cg82-Pg1-Ps55 | Cg82-Pg1-Ps56 |
| Cg82-Pg1-Ps57 | Cg82-Pg1-Ps58 | Cg82-Pg1-Ps59 | Cg82-Pg1-Ps60 |
| Cg82-Pg1-Ps61 | Cg82-Pg1-Ps62 | Cg82-Pg1-Ps63 | Cg82-Pg1-Ps64 |
| Cg82-Pg1-Ps65 | Cg82-Pg1-Ps66 | Cg82-Pg1-Ps67 | Cg82-Pg1-Ps68 |
| Cg82-Pg1-Ps69 | Cg82-Pg1-Ps70 | Cg82-Pg1-Ps71 | Cg82-Pg1-Ps72 |
| Cg82-Pg1-Ps73 | Cg82-Pg1-Ps74 | Cg82-Pg1-Ps75 | Cg82-Pg1-Ps76 |
| Cg82-Pg1-Ps77 | Cg82-Pg1-Ps78 | Cg82-Pg1-Ps79 | Cg82-Pg1-Ps80 |
| Cg82-Pg1-Ps81 | Cg82-Pg1-Ps82 | Cg82-Pg1-Ps83 | Cg82-Pg1-Ps84 |
| Cg82-Pg1-Ps85 | Cg82-Pg1-Ps86 | Cg82-Pg1-Ps87 | Cg82-Pg1-Ps88 |
| Cg82-Pg1-Ps89 | Cg82-Pg1-Ps90 | Cg82-Pg1-Ps91 | Cg82-Pg1-Ps92 |
| Cg82-Pg1-Ps93 | Cg82-Pg1-Ps94 | Cg82-Pg1-Ps95 | Cg82-Pg1-Ps96 |
| Cg82-Pg1-Ps97 | Cg82-Pg1-Ps98 | Cg82-Pg1-Ps99 | Cg82-Pg1-Ps100 |
| Cg82-Pg1-Ps101 | Cg82-Pg1-Ps102 | Cg82-Pg1-Ps103 | Cg82-Pg1-Ps104 |
| Cg82-Pg1-Ps105 | Cg82-Pg1-Ps106 | Cg82-Pg1-Ps107 | Cg82-Pg1-Ps108 |
| Cg82-Pg1-Ps109 | Cg82-Pg1-Ps110 | Cg82-Pg1-Ps111 | Cg82-Pg1-Ps112 |
| Cg82-Pg1-Ps113 | Cg82-Pg1-Ps114 | Cg82-Pg1-Ps115 | Cg82-Pg1-Ps116 |
| Cg82-Pg1-Ps117 | Cg82-Pg1-Ps118 | Cg82-Pg1-Ps119 | Cg82-Pg1-Ps120 |
| Cg82-Pg1-Ps121 | Cg82-Pg1-Ps122 | Cg82-Pg1-Ps123 | Cg82-Pg1-Ps124 |
| Cg82-Pg1-Ps125 | Cg82-Pg1-Ps126 | Cg82-Pg1-Ps127 | Cg82-Pg1-Ps128 |
| Cg82-Pg1-Ps129 | Cg82-Pg1-Ps130 | Cg82-Pg1-Ps131 | Cg82-Pg1-Ps132 |
| Cg82-Pg1-Ps133 | Cg82-Pg1-Ps134 | Cg82-Pg1-Ps135 | Cg82-Pg1-Ps136 |
| Cg82-Pg1-Ps137 | Cg82-Pg1-Ps138 | Cg82-Pg1-Ps139 | Cg82-Pg1-Ps140 |
| Cg82-Pg1-Ps141 | Cg82-Pg1-Ps142 | Cg82-Pg1-Ps143 | Cg82-Pg1-Ps144 |
| Cg82-Pg1-Ps145 | Cg82-Pg1-Ps146 | Cg82-Pg1-Ps147 | Cg82-Pg1-Ps148 |
| Cg82-Pg1-Ps149 | Cg82-Pg1-Ps150 | Cg82-Pg1-Ps151 | Cg82-Pg1-Ps152 |
| Cg82-Pg1-Ps153 | Cg82-Pg1-Ps154 | Cg82-Pg1-Ps155 | Cg82-Pg1-Ps156 |
| Cg82-Pg1-Ps157 | Cg82-Pg1-Ps158 | Cg82-Pg1-Ps159 | Cg82-Pg1-Ps160 |
| Cg82-Pg1-Ps161 | Cg82-Pg1-Ps162 | Cg82-Pg1-Ps163 | Cg82-Pg1-Ps164 |
| Cg82-Pg1-Ps165 | Cg82-Pg1-Ps166 | Cg82-Pg1-Ps167 | Cg82-Pg1-Ps168 |
| Cg82-Pg1-Ps169 | Cg82-Pg1-Ps170 | Cg82-Pg1-Ps171 | Cg82-Pg1-Ps172 |
| Cg82-Pg1-Ps173 | Cg82-Pg1-Ps174 | Cg82-Pg1-Ps175 | Cg82-Pg1-Ps176 |
| Cg82-Pg1-Ps177 | Cg82-Pg1-Ps178 | Cg82-Pg1-Ps179 | Cg82-Pg1-Ps180 |
| Cg82-Pg1-Ps181 | Cg82-Pg1-Ps182 | Cg82-Pg1-Ps183 | Cg82-Pg1-Ps184 |
| Cg82-Pg1-Ps185 | Cg82-Pg1-Ps186 | Cg82-Pg1-Ps187 | Cg82-Pg1-Ps188 |
| Cg82-Pg1-Ps189 | Cg82-Pg1-Ps190 | Cg82-Pg1-Ps191 | Cg82-Pg1-Ps192 |
| Cg82-Pg1-Ps193 | Cg82-Pg1-Ps194 | Cg82-Pg1-Ps195 | Cg82-Pg1-Ps196 |
| Cg82-Pg1-Ps197 | Cg82-Pg1-Ps198 | Cg82-Pg1-Ps199 | Cg82-Pg1-Ps200 |
| Cg82-Pg1-Ps201 | Cg82-Pg1-Ps202 | Cg82-Pg1-Ps203 | Cg82-Pg1-Ps204 |
| Cg82-Pg1-Ps205 | Cg82-Pg1-Ps206 | Cg82-Pg1-Ps207 | Cg82-Pg1-Ps208 |
| Cg82-Pg1-Ps209 | Cg82-Pg1-Ps210 | Cg82-Pg1-Ps211 | Cg82-Pg1-Ps212 |
| Cg82-Pg1-Ps213 | Cg82-Pg1-Ps214 | Cg82-Pg1-Ps215 | Cg82-Pg1-Ps216 |
| Cg82-Pg1-Ps217 | Cg82-Pg1-Ps218 | Cg82-Pg1-Ps219 | Cg82-Pg1-Ps220 |
| Cg82-Pg1-Ps221 | Cg82-Pg1-Ps222 | Cg82-Pg1-Ps223 | Cg82-Pg1-Ps224 |
| Cg82-Pg1-Ps225 | Cg82-Pg1-Ps226 | Cg82-Pg1-Ps227 | Cg82-Pg1-Ps228 |
| Cg82-Pg1-Ps229 | Cg82-Pg1-Ps230 | Cg82-Pg1-Ps231 | Cg82-Pg1-Ps232 |
| Cg82-Pg1-Ps233 | Cg82-Pg1-Ps234 | Cg82-Pg1-Ps235 | Cg82-Pg1-Ps236 |
| Cg82-Pg1-Ps237 | Cg82-Pg1-Ps238 | Cg82-Pg1-Ps239 | Cg82-Pg1-Ps240 |
| Cg82-Pg1-Ps241 | Cg82-Pg1-Ps242 | Cg82-Pg1-Ps243 | |
| Cg83-Pg1-Ps1 | Cg83-Pg1-Ps2 | Cg83-Pg1-Ps3 | Cg83-Pg1-Ps4 |
| Cg83-Pg1-Ps5 | Cg83-Pg1-Ps6 | Cg83-Pg1-Ps7 | Cg83-Pg1-Ps8 |
| Cg83-Pg1-Ps9 | Cg83-Pg1-Ps10 | Cg83-Pg1-Ps11 | Cg83-Pg1-Ps12 |
| Cg83-Pg1-Ps13 | Cg83-Pg1-Ps14 | Cg83-Pg1-Ps15 | Cg83-Pg1-Ps16 |
| Cg83-Pg1-Ps17 | Cg83-Pg1-Ps18 | Cg83-Pg1-Ps19 | Cg83-Pg1-Ps20 |
| Cg83-Pg1-Ps21 | Cg83-Pg1-Ps22 | Cg83-Pg1-Ps23 | Cg83-Pg1-Ps24 |
| Cg83-Pg1-Ps25 | Cg83-Pg1-Ps26 | Cg83-Pg1-Ps27 | Cg83-Pg1-Ps28 |
| Cg83-Pg1-Ps29 | Cg83-Pg1-Ps30 | Cg83-Pg1-Ps31 | Cg83-Pg1-Ps32 |
| Cg83-Pg1-Ps33 | Cg83-Pg1-Ps34 | Cg83-Pg1-Ps35 | Cg83-Pg1-Ps36 |
| Cg83-Pg1-Ps37 | Cg83-Pg1-Ps38 | Cg83-Pg1-Ps39 | Cg83-Pg1-Ps40 |
| Cg83-Pg1-Ps41 | Cg83-Pg1-Ps42 | Cg83-Pg1-Ps43 | Cg83-Pg1-Ps44 |
| Cg83-Pg1-Ps45 | Cg83-Pg1-Ps46 | Cg83-Pg1-Ps47 | Cg83-Pg1-Ps48 |
| Cg83-Pg1-Ps49 | Cg83-Pg1-Ps50 | Cg83-Pg1-Ps51 | Cg83-Pg1-Ps52 |
| Cg83-Pg1-Ps53 | Cg83-Pg1-Ps54 | Cg83-Pg1-Ps55 | Cg83-Pg1-Ps56 |
| Cg83-Pg1-Ps57 | Cg83-Pg1-Ps58 | Cg83-Pg1-Ps59 | Cg83-Pg1-Ps60 |
| Cg83-Pg1-Ps61 | Cg83-Pg1-Ps62 | Cg83-Pg1-Ps63 | Cg83-Pg1-Ps64 |
| Cg83-Pg1-Ps65 | Cg83-Pg1-Ps66 | Cg83-Pg1-Ps67 | Cg83-Pg1-Ps68 |
| Cg83-Pg1-Ps69 | Cg83-Pg1-Ps70 | Cg83-Pg1-Ps71 | Cg83-Pg1-Ps72 |
| Cg83-Pg1-Ps73 | Cg83-Pg1-Ps74 | Cg83-Pg1-Ps75 | Cg83-Pg1-Ps76 |
| Cg83-Pg1-Ps77 | Cg83-Pg1-Ps78 | Cg83-Pg1-Ps79 | Cg83-Pg1-Ps80 |
| Cg83-Pg1-Ps81 | Cg83-Pg1-Ps82 | Cg83-Pg1-Ps83 | Cg83-Pg1-Ps84 |
| Cg83-Pg1-Ps85 | Cg83-Pg1-Ps86 | Cg83-Pg1-Ps87 | Cg83-Pg1-Ps88 |
| Cg83-Pg1-Ps89 | Cg83-Pg1-Ps90 | Cg83-Pg1-Ps91 | Cg83-Pg1-Ps92 |
| Cg83-Pg1-Ps93 | Cg83-Pg1-Ps94 | Cg83-Pg1-Ps95 | Cg83-Pg1-Ps96 |
| Cg83-Pg1-Ps97 | Cg83-Pg1-Ps98 | Cg83-Pg1-Ps99 | Cg83-Pg1-Ps100 |
| Cg83-Pg1-Ps101 | Cg83-Pg1-Ps102 | Cg83-Pg1-Ps103 | Cg83-Pg1-Ps104 |
| Cg83-Pg1-Ps105 | Cg83-Pg1-Ps106 | Cg83-Pg1-Ps107 | Cg83-Pg1-Ps108 |
| Cg83-Pg1-Ps109 | Cg83-Pg1-Ps110 | Cg83-Pg1-Ps111 | Cg83-Pg1-Ps112 |
| Cg83-Pg1-Ps113 | Cg83-Pg1-Ps114 | Cg83-Pg1-Ps115 | Cg83-Pg1-Ps116 |
| Cg83-Pg1-Ps117 | Cg83-Pg1-Ps118 | Cg83-Pg1-Ps119 | Cg83-Pg1-Ps120 |
| Cg83-Pg1-Ps121 | Cg83-Pg1-Ps122 | Cg83-Pg1-Ps123 | Cg83-Pg1-Ps124 |
| Cg83-Pg1-Ps125 | Cg83-Pg1-Ps126 | Cg83-Pg1-Ps127 | Cg83-Pg1-Ps128 |
| Cg83-Pg1-Ps129 | Cg83-Pg1-Ps130 | Cg83-Pg1-Ps131 | Cg83-Pg1-Ps132 |
| Cg83-Pg1-Ps133 | Cg83-Pg1-Ps134 | Cg83-Pg1-Ps135 | Cg83-Pg1-Ps136 |
| Cg83-Pg1-Ps137 | Cg83-Pg1-Ps138 | Cg83-Pg1-Ps139 | Cg83-Pg1-Ps140 |
| Cg83-Pg1-Ps141 | Cg83-Pg1-Ps142 | Cg83-Pg1-Ps143 | Cg83-Pg1-Ps144 |
| Cg83-Pg1-Ps145 | Cg83-Pg1-Ps146 | Cg83-Pg1-Ps147 | Cg83-Pg1-Ps148 |
| Cg83-Pg1-Ps149 | Cg83-Pg1-Ps150 | Cg83-Pg1-Ps151 | Cg83-Pg1-Ps152 |
| Cg83-Pg1-Ps153 | Cg83-Pg1-Ps154 | Cg83-Pg1-Ps155 | Cg83-Pg1-Ps156 |
| Cg83-Pg1-Ps157 | Cg83-Pg1-Ps158 | Cg83-Pg1-Ps159 | Cg83-Pg1-Ps160 |
| Cg83-Pg1-Ps161 | Cg83-Pg1-Ps162 | Cg83-Pg1-Ps163 | Cg83-Pg1-Ps164 |
| Cg83-Pg1-Ps165 | Cg83-Pg1-Ps166 | Cg83-Pg1-Ps167 | Cg83-Pg1-Ps168 |
| Cg83-Pg1-Ps169 | Cg83-Pg1-Ps170 | Cg83-Pg1-Ps171 | Cg83-Pg1-Ps172 |
| Cg83-Pg1-Ps173 | Cg83-Pg1-Ps174 | Cg83-Pg1-Ps175 | Cg83-Pg1-Ps176 |
| Cg83-Pg1-Ps177 | Cg83-Pg1-Ps178 | Cg83-Pg1-Ps179 | Cg83-Pg1-Ps180 |
| Cg83-Pg1-Ps181 | Cg83-Pg1-Ps182 | Cg83-Pg1-Ps183 | Cg83-Pg1-Ps184 |
| Cg83-Pg1-Ps185 | Cg83-Pg1-Ps186 | Cg83-Pg1-Ps187 | Cg83-Pg1-Ps188 |
| Cg83-Pg1-Ps189 | Cg83-Pg1-Ps190 | Cg83-Pg1-Ps191 | Cg83-Pg1-Ps192 |
| Cg83-Pg1-Ps193 | Cg83-Pg1-Ps194 | Cg83-Pg1-Ps195 | Cg83-Pg1-Ps196 |
| Cg83-Pg1-Ps197 | Cg83-Pg1-Ps198 | Cg83-Pg1-Ps199 | Cg83-Pg1-Ps200 |
| Cg83-Pg1-Ps201 | Cg83-Pg1-Ps202 | Cg83-Pg1-Ps203 | Cg83-Pg1-Ps204 |
| Cg83-Pg1-Ps205 | Cg83-Pg1-Ps206 | Cg83-Pg1-Ps207 | Cg83-Pg1-Ps208 |
| Cg83-Pg1-Ps209 | Cg83-Pg1-Ps210 | Cg83-Pg1-Ps211 | Cg83-Pg1-Ps212 |
| Cg83-Pg1-Ps213 | Cg83-Pg1-Ps214 | Cg83-Pg1-Ps215 | Cg83-Pg1-Ps216 |
| Cg83-Pg1-Ps217 | Cg83-Pg1-Ps218 | Cg83-Pg1-Ps219 | Cg83-Pg1-Ps220 |
| Cg83-Pg1-Ps221 | Cg83-Pg1-Ps222 | Cg83-Pg1-Ps223 | Cg83-Pg1-Ps224 |
| Cg83-Pg1-Ps225 | Cg83-Pg1-Ps226 | Cg83-Pg1-Ps227 | Cg83-Pg1-Ps228 |
| Cg83-Pg1-Ps229 | Cg83-Pg1-Ps230 | Cg83-Pg1-Ps231 | Cg83-Pg1-Ps232 |
| Cg83-Pg1-Ps233 | Cg83-Pg1-Ps234 | Cg83-Pg1-Ps235 | Cg83-Pg1-Ps236 |
| Cg83-Pg1-Ps237 | Cg83-Pg1-Ps238 | Cg83-Pg1-Ps239 | Cg83-Pg1-Ps240 |
| Cg83-Pg1-Ps241 | Cg83-Pg1-Ps242 | Cg83-Pg1-Ps243 | |
| Cg84-Pg1-Ps1 | Cg84-Pg1-Ps2 | Cg84-Pg1-Ps3 | Cg84-Pg1-Ps4 |
| Cg84-Pg1-Ps5 | Cg84-Pg1-Ps6 | Cg84-Pg1-Ps7 | Cg84-Pg1-Ps8 |
| Cg84-Pg1-Ps9 | Cg84-Pg1-Ps10 | Cg84-Pg1-Ps11 | Cg84-Pg1-Ps12 |
| Cg84-Pg1-Ps13 | Cg84-Pg1-Ps14 | Cg84-Pg1-Ps15 | Cg84-Pg1-Ps16 |
| Cg84-Pg1-Ps17 | Cg84-Pg1-Ps18 | Cg84-Pg1-Ps19 | Cg84-Pg1-Ps20 |
| Cg84-Pg1-Ps21 | Cg84-Pg1-Ps22 | Cg84-Pg1-Ps23 | Cg84-Pg1-Ps24 |
| Cg84-Pg1-Ps25 | Cg84-Pg1-Ps26 | Cg84-Pg1-Ps27 | Cg84-Pg1-Ps28 |
| Cg84-Pg1-Ps29 | Cg84-Pg1-Ps30 | Cg84-Pg1-Ps31 | Cg84-Pg1-Ps32 |
| Cg84-Pg1-Ps33 | Cg84-Pg1-Ps34 | Cg84-Pg1-Ps35 | Cg84-Pg1-Ps36 |
| Cg84-Pg1-Ps37 | Cg84-Pg1-Ps38 | Cg84-Pg1-Ps39 | Cg84-Pg1-Ps40 |
| Cg84-Pg1-Ps41 | Cg84-Pg1-Ps42 | Cg84-Pg1-Ps43 | Cg84-Pg1-Ps44 |
| Cg84-Pg1-Ps45 | Cg84-Pg1-Ps46 | Cg84-Pg1-Ps47 | Cg84-Pg1-Ps48 |
| Cg84-Pg1-Ps49 | Cg84-Pg1-Ps50 | Cg84-Pg1-Ps51 | Cg84-Pg1-Ps52 |
| Cg84-Pg1-Ps53 | Cg84-Pg1-Ps54 | Cg84-Pg1-Ps55 | Cg84-Pg1-Ps56 |
| Cg84-Pg1-Ps57 | Cg84-Pg1-Ps58 | Cg84-Pg1-Ps59 | Cg84-Pg1-Ps60 |
| Cg84-Pg1-Ps61 | Cg84-Pg1-Ps62 | Cg84-Pg1-Ps63 | Cg84-Pg1-Ps64 |
| Cg84-Pg1-Ps65 | Cg84-Pg1-Ps66 | Cg84-Pg1-Ps67 | Cg84-Pg1-Ps68 |
| Cg84-Pg1-Ps69 | Cg84-Pg1-Ps70 | Cg84-Pg1-Ps71 | Cg84-Pg1-Ps72 |
| Cg84-Pg1-Ps73 | Cg84-Pg1-Ps74 | Cg84-Pg1-Ps75 | Cg84-Pg1-Ps76 |
| Cg84-Pg1-Ps77 | Cg84-Pg1-Ps78 | Cg84-Pg1-Ps79 | Cg84-Pg1-Ps80 |
| Cg84-Pg1-Ps81 | Cg84-Pg1-Ps82 | Cg84-Pg1-Ps83 | Cg84-Pg1-Ps84 |
| Cg84-Pg1-Ps85 | Cg84-Pg1-Ps86 | Cg84-Pg1-Ps87 | Cg84-Pg1-Ps88 |
| Cg84-Pg1-Ps89 | Cg84-Pg1-Ps90 | Cg84-Pg1-Ps91 | Cg84-Pg1-Ps92 |
| Cg84-Pg1-Ps93 | Cg84-Pg1-Ps94 | Cg84-Pg1-Ps95 | Cg84-Pg1-Ps96 |
| Cg84-Pg1-Ps97 | Cg84-Pg1-Ps98 | Cg84-Pg1-Ps99 | Cg84-Pg1-Ps100 |
| Cg84-Pg1-Ps101 | Cg84-Pg1-Ps102 | Cg84-Pg1-Ps103 | Cg84-Pg1-Ps104 |
| Cg84-Pg1-Ps105 | Cg84-Pg1-Ps106 | Cg84-Pg1-Ps107 | Cg84-Pg1-Ps108 |
| Cg84-Pg1-Ps109 | Cg84-Pg1-Ps110 | Cg84-Pg1-Ps111 | Cg84-Pg1-Ps112 |
| Cg84-Pg1-Ps113 | Cg84-Pg1-Ps114 | Cg84-Pg1-Ps115 | Cg84-Pg1-Ps116 |
| Cg84-Pg1-Ps117 | Cg84-Pg1-Ps118 | Cg84-Pg1-Ps119 | Cg84-Pg1-Ps120 |
| Cg84-Pg1-Ps121 | Cg84-Pg1-Ps122 | Cg84-Pg1-Ps123 | Cg84-Pg1-Ps124 |
| Cg84-Pg1-Ps125 | Cg84-Pg1-Ps126 | Cg84-Pg1-Ps127 | Cg84-Pg1-Ps128 |
| Cg84-Pg1-Ps129 | Cg84-Pg1-Ps130 | Cg84-Pg1-Ps131 | Cg84-Pg1-Ps132 |
| Cg84-Pg1-Ps133 | Cg84-Pg1-Ps134 | Cg84-Pg1-Ps135 | Cg84-Pg1-Ps136 |
| Cg84-Pg1-Ps137 | Cg84-Pg1-Ps138 | Cg84-Pg1-Ps139 | Cg84-Pg1-Ps140 |
| Cg84-Pg1-Ps141 | Cg84-Pg1-Ps142 | Cg84-Pg1-Ps143 | Cg84-Pg1-Ps144 |
| Cg84-Pg1-Ps145 | Cg84-Pg1-Ps146 | Cg84-Pg1-Ps147 | Cg84-Pg1-Ps148 |
| Cg84-Pg1-Ps149 | Cg84-Pg1-Ps150 | Cg84-Pg1-Ps151 | Cg84-Pg1-Ps152 |
| Cg84-Pg1-Ps153 | Cg84-Pg1-Ps154 | Cg84-Pg1-Ps155 | Cg84-Pg1-Ps156 |
| Cg84-Pg1-Ps157 | Cg84-Pg1-Ps158 | Cg84-Pg1-Ps159 | Cg84-Pg1-Ps160 |
| Cg84-Pg1-Ps161 | Cg84-Pg1-Ps162 | Cg84-Pg1-Ps163 | Cg84-Pg1-Ps164 |
| Cg84-Pg1-Ps165 | Cg84-Pg1-Ps166 | Cg84-Pg1-Ps167 | Cg84-Pg1-Ps168 |
| Cg84-Pg1-Ps169 | Cg84-Pg1-Ps170 | Cg84-Pg1-Ps171 | Cg84-Pg1-Ps172 |
| Cg84-Pg1-Ps173 | Cg84-Pg1-Ps174 | Cg84-Pg1-Ps175 | Cg84-Pg1-Ps176 |
| Cg84-Pg1-Ps177 | Cg84-Pg1-Ps178 | Cg84-Pg1-Ps179 | Cg84-Pg1-Ps180 |
| Cg84-Pg1-Ps181 | Cg84-Pg1-Ps182 | Cg84-Pg1-Ps183 | Cg84-Pg1-Ps184 |
| Cg84-Pg1-Ps185 | Cg84-Pg1-Ps186 | Cg84-Pg1-Ps187 | Cg84-Pg1-Ps188 |
| Cg84-Pg1-Ps189 | Cg84-Pg1-Ps190 | Cg84-Pg1-Ps191 | Cg84-Pg1-Ps192 |
| Cg84-Pg1-Ps193 | Cg84-Pg1-Ps194 | Cg84-Pg1-Ps195 | Cg84-Pg1-Ps196 |
| Cg84-Pg1-Ps197 | Cg84-Pg1-Ps198 | Cg84-Pg1-Ps199 | Cg84-Pg1-Ps200 |
| Cg84-Pg1-Ps201 | Cg84-Pg1-Ps202 | Cg84-Pg1-Ps203 | Cg84-Pg1-Ps204 |
| Cg84-Pg1-Ps205 | Cg84-Pg1-Ps206 | Cg84-Pg1-Ps207 | Cg84-Pg1-Ps208 |
| Cg84-Pg1-Ps209 | Cg84-Pg1-Ps210 | Cg84-Pg1-Ps211 | Cg84-Pg1-Ps212 |
| Cg84-Pg1-Ps213 | Cg84-Pg1-Ps214 | Cg84-Pg1-Ps215 | Cg84-Pg1-Ps216 |
| Cg84-Pg1-Ps217 | Cg84-Pg1-Ps218 | Cg84-Pg1-Ps219 | Cg84-Pg1-Ps220 |
| Cg84-Pg1-Ps221 | Cg84-Pg1-Ps222 | Cg84-Pg1-Ps223 | Cg84-Pg1-Ps224 |
| Cg84-Pg1-Ps225 | Cg84-Pg1-Ps226 | Cg84-Pg1-Ps227 | Cg84-Pg1-Ps228 |
| Cg84-Pg1-Ps229 | Cg84-Pg1-Ps230 | Cg84-Pg1-Ps231 | Cg84-Pg1-Ps232 |
| Cg84-Pg1-Ps233 | Cg84-Pg1-Ps234 | Cg84-Pg1-Ps235 | Cg84-Pg1-Ps236 |
| Cg84-Pg1-Ps237 | Cg84-Pg1-Ps238 | Cg84-Pg1-Ps239 | Cg84-Pg1-Ps240 |
| Cg84-Pg1-Ps241 | Cg84-Pg1-Ps242 | Cg84-Pg1-Ps243 | |
| Cg85-Pg1-Ps1 | Cg85-Pg1-Ps2 | Cg85-Pg1-Ps3 | Cg85-Pg1-Ps4 |
| Cg85-Pg1-Ps5 | Cg85-Pg1-Ps6 | Cg85-Pg1-Ps7 | Cg85-Pg1-Ps8 |
| Cg85-Pg1-Ps9 | Cg85-Pg1-Ps10 | Cg85-Pg1-Ps11 | Cg85-Pg1-Ps12 |
| Cg85-Pg1-Ps13 | Cg85-Pg1-Ps14 | Cg85-Pg1-Ps15 | Cg85-Pg1-Ps16 |
| Cg85-Pg1-Ps17 | Cg85-Pg1-Ps18 | Cg85-Pg1-Ps19 | Cg85-Pg1-Ps20 |
| Cg85-Pg1-Ps21 | Cg85-Pg1-Ps22 | Cg85-Pg1-Ps23 | Cg85-Pg1-Ps24 |
| Cg85-Pg1-Ps25 | Cg85-Pg1-Ps26 | Cg85-Pg1-Ps27 | Cg85-Pg1-Ps28 |
| Cg85-Pg1-Ps29 | Cg85-Pg1-Ps30 | Cg85-Pg1-Ps31 | Cg85-Pg1-Ps32 |
| Cg85-Pg1-Ps33 | Cg85-Pg1-Ps34 | Cg85-Pg1-Ps35 | Cg85-Pg1-Ps36 |
| Cg85-Pg1-Ps37 | Cg85-Pg1-Ps38 | Cg85-Pg1-Ps39 | Cg85-Pg1-Ps40 |
| Cg85-Pg1-Ps41 | Cg85-Pg1-Ps42 | Cg85-Pg1-Ps43 | Cg85-Pg1-Ps44 |
| Cg85-Pg1-Ps45 | Cg85-Pg1-Ps46 | Cg85-Pg1-Ps47 | Cg85-Pg1-Ps48 |
| Cg85-Pg1-Ps49 | Cg85-Pg1-Ps50 | Cg85-Pg1-Ps51 | Cg85-Pg1-Ps52 |
| Cg85-Pg1-Ps53 | Cg85-Pg1-Ps54 | Cg85-Pg1-Ps55 | Cg85-Pg1-Ps56 |
| Cg85-Pg1-Ps57 | Cg85-Pg1-Ps58 | Cg85-Pg1-Ps59 | Cg85-Pg1-Ps60 |
| Cg85-Pg1-Ps61 | Cg85-Pg1-Ps62 | Cg85-Pg1-Ps63 | Cg85-Pg1-Ps64 |
| Cg85-Pg1-Ps65 | Cg85-Pg1-Ps66 | Cg85-Pg1-Ps67 | Cg85-Pg1-Ps68 |
| Cg85-Pg1-Ps69 | Cg85-Pg1-Ps70 | Cg85-Pg1-Ps71 | Cg85-Pg1-Ps72 |
| Cg85-Pg1-Ps73 | Cg85-Pg1-Ps74 | Cg85-Pg1-Ps75 | Cg85-Pg1-Ps76 |
| Cg85-Pg1-Ps77 | Cg85-Pg1-Ps78 | Cg85-Pg1-Ps79 | Cg85-Pg1-Ps80 |
| Cg85-Pg1-Ps81 | Cg85-Pg1-Ps82 | Cg85-Pg1-Ps83 | Cg85-Pg1-Ps84 |
| Cg85-Pg1-Ps85 | Cg85-Pg1-Ps86 | Cg85-Pg1-Ps87 | Cg85-Pg1-Ps88 |
| Cg85-Pg1-Ps89 | Cg85-Pg1-Ps90 | Cg85-Pg1-Ps91 | Cg85-Pg1-Ps92 |
| Cg85-Pg1-Ps93 | Cg85-Pg1-Ps94 | Cg85-Pg1-Ps95 | Cg85-Pg1-Ps96 |
| Cg85-Pg1-Ps97 | Cg85-Pg1-Ps98 | Cg85-Pg1-Ps99 | Cg85-Pg1-Ps100 |
| Cg85-Pg1-Ps101 | Cg85-Pg1-Ps102 | Cg85-Pg1-Ps103 | Cg85-Pg1-Ps104 |
| Cg85-Pg1-Ps105 | Cg85-Pg1-Ps106 | Cg85-Pg1-Ps107 | Cg85-Pg1-Ps108 |
| Cg85-Pg1-Ps109 | Cg85-Pg1-Ps110 | Cg85-Pg1-Ps111 | Cg85-Pg1-Ps112 |
| Cg85-Pg1-Ps113 | Cg85-Pg1-Ps114 | Cg85-Pg1-Ps115 | Cg85-Pg1-Ps116 |
| Cg85-Pg1-Ps117 | Cg85-Pg1-Ps118 | Cg85-Pg1-Ps119 | Cg85-Pg1-Ps120 |
| Cg85-Pg1-Ps121 | Cg85-Pg1-Ps122 | Cg85-Pg1-Ps123 | Cg85-Pg1-Ps124 |
| Cg85-Pg1-Ps125 | Cg85-Pg1-Ps126 | Cg85-Pg1-Ps127 | Cg85-Pg1-Ps128 |
| Cg85-Pg1-Ps129 | Cg85-Pg1-Ps130 | Cg85-Pg1-Ps131 | Cg85-Pg1-Ps132 |
| Cg85-Pg1-Ps133 | Cg85-Pg1-Ps134 | Cg85-Pg1-Ps135 | Cg85-Pg1-Ps136 |
| Cg85-Pg1-Ps137 | Cg85-Pg1-Ps138 | Cg85-Pg1-Ps139 | Cg85-Pg1-Ps140 |
| Cg85-Pg1-Ps141 | Cg85-Pg1-Ps142 | Cg85-Pg1-Ps143 | Cg85-Pg1-Ps144 |
| Cg85-Pg1-Ps145 | Cg85-Pg1-Ps146 | Cg85-Pg1-Ps147 | Cg85-Pg1-Ps148 |
| Cg85-Pg1-Ps149 | Cg85-Pg1-Ps150 | Cg85-Pg1-Ps151 | Cg85-Pg1-Ps152 |
| Cg85-Pg1-Ps153 | Cg85-Pg1-Ps154 | Cg85-Pg1-Ps155 | Cg85-Pg1-Ps156 |
| Cg85-Pg1-Ps157 | Cg85-Pg1-Ps158 | Cg85-Pg1-Ps159 | Cg85-Pg1-Ps160 |
| Cg85-Pg1-Ps161 | Cg85-Pg1-Ps162 | Cg85-Pg1-Ps163 | Cg85-Pg1-Ps164 |
| Cg85-Pg1-Ps165 | Cg85-Pg1-Ps166 | Cg85-Pg1-Ps167 | Cg85-Pg1-Ps168 |
| Cg85-Pg1-Ps169 | Cg85-Pg1-Ps170 | Cg85-Pg1-Ps171 | Cg85-Pg1-Ps172 |
| Cg85-Pg1-Ps173 | Cg85-Pg1-Ps174 | Cg85-Pg1-Ps175 | Cg85-Pg1-Ps176 |
| Cg85-Pg1-Ps177 | Cg85-Pg1-Ps178 | Cg85-Pg1-Ps179 | Cg85-Pg1-Ps180 |
| Cg85-Pg1-Ps181 | Cg85-Pg1-Ps182 | Cg85-Pg1-Ps183 | Cg85-Pg1-Ps184 |
| Cg85-Pg1-Ps185 | Cg85-Pg1-Ps186 | Cg85-Pg1-Ps187 | Cg85-Pg1-Ps188 |
| Cg85-Pg1-Ps189 | Cg85-Pg1-Ps190 | Cg85-Pg1-Ps191 | Cg85-Pg1-Ps192 |
| Cg85-Pg1-Ps193 | Cg85-Pg1-Ps194 | Cg85-Pg1-Ps195 | Cg85-Pg1-Ps196 |
| Cg85-Pg1-Ps197 | Cg85-Pg1-Ps198 | Cg85-Pg1-Ps199 | Cg85-Pg1-Ps200 |
| Cg85-Pg1-Ps201 | Cg85-Pg1-Ps202 | Cg85-Pg1-Ps203 | Cg85-Pg1-Ps204 |
| Cg85-Pg1-Ps205 | Cg85-Pg1-Ps206 | Cg85-Pg1-Ps207 | Cg85-Pg1-Ps208 |
| Cg85-Pg1-Ps209 | Cg85-Pg1-Ps210 | Cg85-Pg1-Ps211 | Cg85-Pg1-Ps212 |
| Cg85-Pg1-Ps213 | Cg85-Pg1-Ps214 | Cg85-Pg1-Ps215 | Cg85-Pg1-Ps216 |
| Cg85-Pg1-Ps217 | Cg85-Pg1-Ps218 | Cg85-Pg1-Ps219 | Cg85-Pg1-Ps220 |
| Cg85-Pg1-Ps221 | Cg85-Pg1-Ps222 | Cg85-Pg1-Ps223 | Cg85-Pg1-Ps224 |
| Cg85-Pg1-Ps225 | Cg85-Pg1-Ps226 | Cg85-Pg1-Ps227 | Cg85-Pg1-Ps228 |
| Cg85-Pg1-Ps229 | Cg85-Pg1-Ps230 | Cg85-Pg1-Ps231 | Cg85-Pg1-Ps232 |
| Cg85-Pg1-Ps233 | Cg85-Pg1-Ps234 | Cg85-Pg1-Ps235 | Cg85-Pg1-Ps236 |
| Cg85-Pg1-Ps237 | Cg85-Pg1-Ps238 | Cg85-Pg1-Ps239 | Cg85-Pg1-Ps240 |
| Cg85-Pg1-Ps241 | Cg85-Pg1-Ps242 | Cg85-Pg1-Ps243 | |
| Cg86-Pg1-Ps1 | Cg86-Pg1-Ps2 | Cg86-Pg1-Ps3 | Cg86-Pg1-Ps4 |
| Cg86-Pg1-Ps5 | Cg86-Pg1-Ps6 | Cg86-Pg1-Ps7 | Cg86-Pg1-Ps8 |
| Cg86-Pg1-Ps9 | Cg86-Pg1-Ps10 | Cg86-Pg1-Ps11 | Cg86-Pg1-Ps12 |
| Cg86-Pg1-Ps13 | Cg86-Pg1-Ps14 | Cg86-Pg1-Ps15 | Cg86-Pg1-Ps16 |
| Cg86-Pg1-Ps17 | Cg86-Pg1-Ps18 | Cg86-Pg1-Ps19 | Cg86-Pg1-Ps20 |
| Cg86-Pg1-Ps21 | Cg86-Pg1-Ps22 | Cg86-Pg1-Ps23 | Cg86-Pg1-Ps24 |
| Cg86-Pg1-Ps25 | Cg86-Pg1-Ps26 | Cg86-Pg1-Ps27 | Cg86-Pg1-Ps28 |
| Cg86-Pg1-Ps29 | Cg86-Pg1-Ps30 | Cg86-Pg1-Ps31 | Cg86-Pg1-Ps32 |
| Cg86-Pg1-Ps33 | Cg86-Pg1-Ps34 | Cg86-Pg1-Ps35 | Cg86-Pg1-Ps36 |
| Cg86-Pg1-Ps37 | Cg86-Pg1-Ps38 | Cg86-Pg1-Ps39 | Cg86-Pg1-Ps40 |
| Cg86-Pg1-Ps41 | Cg86-Pg1-Ps42 | Cg86-Pg1-Ps43 | Cg86-Pg1-Ps44 |
| Cg86-Pg1-Ps45 | Cg86-Pg1-Ps46 | Cg86-Pg1-Ps47 | Cg86-Pg1-Ps48 |
| Cg86-Pg1-Ps49 | Cg86-Pg1-Ps50 | Cg86-Pg1-Ps51 | Cg86-Pg1-Ps52 |
| Cg86-Pg1-Ps53 | Cg86-Pg1-Ps54 | Cg86-Pg1-Ps55 | Cg86-Pg1-Ps56 |
| Cg86-Pg1-Ps57 | Cg86-Pg1-Ps58 | Cg86-Pg1-Ps59 | Cg86-Pg1-Ps60 |
| Cg86-Pg1-Ps61 | Cg86-Pg1-Ps62 | Cg86-Pg1-Ps63 | Cg86-Pg1-Ps64 |
| Cg86-Pg1-Ps65 | Cg86-Pg1-Ps66 | Cg86-Pg1-Ps67 | Cg86-Pg1-Ps68 |
| Cg86-Pg1-Ps69 | Cg86-Pg1-Ps70 | Cg86-Pg1-Ps71 | Cg86-Pg1-Ps72 |
| Cg86-Pg1-Ps73 | Cg86-Pg1-Ps74 | Cg86-Pg1-Ps75 | Cg86-Pg1-Ps76 |
| Cg86-Pg1-Ps77 | Cg86-Pg1-Ps78 | Cg86-Pg1-Ps79 | Cg86-Pg1-Ps80 |
| Cg86-Pg1-Ps81 | Cg86-Pg1-Ps82 | Cg86-Pg1-Ps83 | Cg86-Pg1-Ps84 |
| Cg86-Pg1-Ps85 | Cg86-Pg1-Ps86 | Cg86-Pg1-Ps87 | Cg86-Pg1-Ps88 |
| Cg86-Pg1-Ps89 | Cg86-Pg1-Ps90 | Cg86-Pg1-Ps91 | Cg86-Pg1-Ps92 |
| Cg86-Pg1-Ps93 | Cg86-Pg1-Ps94 | Cg86-Pg1-Ps95 | Cg86-Pg1-Ps96 |
| Cg86-Pg1-Ps97 | Cg86-Pg1-Ps98 | Cg86-Pg1-Ps99 | Cg86-Pg1-Ps100 |
| Cg86-Pg1-Ps101 | Cg86-Pg1-Ps102 | Cg86-Pg1-Ps103 | Cg86-Pg1-Ps104 |
| Cg86-Pg1-Ps105 | Cg86-Pg1-Ps106 | Cg86-Pg1-Ps107 | Cg86-Pg1-Ps108 |
| Cg86-Pg1-Ps109 | Cg86-Pg1-Ps110 | Cg86-Pg1-Ps111 | Cg86-Pg1-Ps112 |
| Cg86-Pg1-Ps113 | Cg86-Pg1-Ps114 | Cg86-Pg1-Ps115 | Cg86-Pg1-Ps116 |
| Cg86-Pg1-Ps117 | Cg86-Pg1-Ps118 | Cg86-Pg1-Ps119 | Cg86-Pg1-Ps120 |
| Cg86-Pg1-Ps121 | Cg86-Pg1-Ps122 | Cg86-Pg1-Ps123 | Cg86-Pg1-Ps124 |
| Cg86-Pg1-Ps125 | Cg86-Pg1-Ps126 | Cg86-Pg1-Ps127 | Cg86-Pg1-Ps128 |
| Cg86-Pg1-Ps129 | Cg86-Pg1-Ps130 | Cg86-Pg1-Ps131 | Cg86-Pg1-Ps132 |
| Cg86-Pg1-Ps133 | Cg86-Pg1-Ps134 | Cg86-Pg1-Ps135 | Cg86-Pg1-Ps136 |
| Cg86-Pg1-Ps137 | Cg86-Pg1-Ps138 | Cg86-Pg1-Ps139 | Cg86-Pg1-Ps140 |
| Cg86-Pg1-Ps141 | Cg86-Pg1-Ps142 | Cg86-Pg1-Ps143 | Cg86-Pg1-Ps144 |
| Cg86-Pg1-Ps145 | Cg86-Pg1-Ps146 | Cg86-Pg1-Ps147 | Cg86-Pg1-Ps148 |
| Cg86-Pg1-Ps149 | Cg86-Pg1-Ps150 | Cg86-Pg1-Ps151 | Cg86-Pg1-Ps152 |
| Cg86-Pg1-Ps153 | Cg86-Pg1-Ps154 | Cg86-Pg1-Ps155 | Cg86-Pg1-Ps156 |
| Cg86-Pg1-Ps157 | Cg86-Pg1-Ps158 | Cg86-Pg1-Ps159 | Cg86-Pg1-Ps160 |
| Cg86-Pg1-Ps161 | Cg86-Pg1-Ps162 | Cg86-Pg1-Ps163 | Cg86-Pg1-Ps164 |
| Cg86-Pg1-Ps165 | Cg86-Pg1-Ps166 | Cg86-Pg1-Ps167 | Cg86-Pg1-Ps168 |
| Cg86-Pg1-Ps169 | Cg86-Pg1-Ps170 | Cg86-Pg1-Ps171 | Cg86-Pg1-Ps172 |
| Cg86-Pg1-Ps173 | Cg86-Pg1-Ps174 | Cg86-Pg1-Ps175 | Cg86-Pg1-Ps176 |
| Cg86-Pg1-Ps177 | Cg86-Pg1-Ps178 | Cg86-Pg1-Ps179 | Cg86-Pg1-Ps180 |
| Cg86-Pg1-Ps181 | Cg86-Pg1-Ps182 | Cg86-Pg1-Ps183 | Cg86-Pg1-Ps184 |
| Cg86-Pg1-Ps185 | Cg86-Pg1-Ps186 | Cg86-Pg1-Ps187 | Cg86-Pg1-Ps188 |
| Cg86-Pg1-Ps189 | Cg86-Pg1-Ps190 | Cg86-Pg1-Ps191 | Cg86-Pg1-Ps192 |
| Cg86-Pg1-Ps193 | Cg86-Pg1-Ps194 | Cg86-Pg1-Ps195 | Cg86-Pg1-Ps196 |
| Cg86-Pg1-Ps197 | Cg86-Pg1-Ps198 | Cg86-Pg1-Ps199 | Cg86-Pg1-Ps200 |
| Cg86-Pg1-Ps201 | Cg86-Pg1-Ps202 | Cg86-Pg1-Ps203 | Cg86-Pg1-Ps204 |
| Cg86-Pg1-Ps205 | Cg86-Pg1-Ps206 | Cg86-Pg1-Ps207 | Cg86-Pg1-Ps208 |
| Cg86-Pg1-Ps209 | Cg86-Pg1-Ps210 | Cg86-Pg1-Ps211 | Cg86-Pg1-Ps212 |
| Cg86-Pg1-Ps213 | Cg86-Pg1-Ps214 | Cg86-Pg1-Ps215 | Cg86-Pg1-Ps216 |
| Cg86-Pg1-Ps217 | Cg86-Pg1-Ps218 | Cg86-Pg1-Ps219 | Cg86-Pg1-Ps220 |
| Cg86-Pg1-Ps221 | Cg86-Pg1-Ps222 | Cg86-Pg1-Ps223 | Cg86-Pg1-Ps224 |
| Cg86-Pg1-Ps225 | Cg86-Pg1-Ps226 | Cg86-Pg1-Ps227 | Cg86-Pg1-Ps228 |
| Cg86-Pg1-Ps229 | Cg86-Pg1-Ps230 | Cg86-Pg1-Ps231 | Cg86-Pg1-Ps232 |
| Cg86-Pg1-Ps233 | Cg86-Pg1-Ps234 | Cg86-Pg1-Ps235 | Cg86-Pg1-Ps236 |
| Cg86-Pg1-Ps237 | Cg86-Pg1-Ps238 | Cg86-Pg1-Ps239 | Cg86-Pg1-Ps240 |
| Cg86-Pg1-Ps241 | Cg86-Pg1-Ps242 | Cg86-Pg1-Ps243 | |
| Cg87-Pg1-Ps1 | Cg87-Pg1-Ps2 | Cg87-Pg1-Ps3 | Cg87-Pg1-Ps4 |
| Cg87-Pg1-Ps5 | Cg87-Pg1-Ps6 | Cg87-Pg1-Ps7 | Cg87-Pg1-Ps8 |
| Cg87-Pg1-Ps9 | Cg87-Pg1-Ps10 | Cg87-Pg1-Ps11 | Cg87-Pg1-Ps12 |
| Cg87-Pg1-Ps13 | Cg87-Pg1-Ps14 | Cg87-Pg1-Ps15 | Cg87-Pg1-Ps16 |
| Cg87-Pg1-Ps17 | Cg87-Pg1-Ps18 | Cg87-Pg1-Ps19 | Cg87-Pg1-Ps20 |
| Cg87-Pg1-Ps21 | Cg87-Pg1-Ps22 | Cg87-Pg1-Ps23 | Cg87-Pg1-Ps24 |
| Cg87-Pg1-Ps25 | Cg87-Pg1-Ps26 | Cg87-Pg1-Ps27 | Cg87-Pg1-Ps28 |
| Cg87-Pg1-Ps29 | Cg87-Pg1-Ps30 | Cg87-Pg1-Ps31 | Cg87-Pg1-Ps32 |
| Cg87-Pg1-Ps33 | Cg87-Pg1-Ps34 | Cg87-Pg1-Ps35 | Cg87-Pg1-Ps36 |
| Cg87-Pg1-Ps37 | Cg87-Pg1-Ps38 | Cg87-Pg1-Ps39 | Cg87-Pg1-Ps40 |
| Cg87-Pg1-Ps41 | Cg87-Pg1-Ps42 | Cg87-Pg1-Ps43 | Cg87-Pg1-Ps44 |
| Cg87-Pg1-Ps45 | Cg87-Pg1-Ps46 | Cg87-Pg1-Ps47 | Cg87-Pg1-Ps48 |
| Cg87-Pg1-Ps49 | Cg87-Pg1-Ps50 | Cg87-Pg1-Ps51 | Cg87-Pg1-Ps52 |
| Cg87-Pg1-Ps53 | Cg87-Pg1-Ps54 | Cg87-Pg1-Ps55 | Cg87-Pg1-Ps56 |
| Cg87-Pg1-Ps57 | Cg87-Pg1-Ps58 | Cg87-Pg1-Ps59 | Cg87-Pg1-Ps60 |
| Cg87-Pg1-Ps61 | Cg87-Pg1-Ps62 | Cg87-Pg1-Ps63 | Cg87-Pg1-Ps64 |
| Cg87-Pg1-Ps65 | Cg87-Pg1-Ps66 | Cg87-Pg1-Ps67 | Cg87-Pg1-Ps68 |
| Cg87-Pg1-Ps69 | Cg87-Pg1-Ps70 | Cg87-Pg1-Ps71 | Cg87-Pg1-Ps72 |
| Cg87-Pg1-Ps73 | Cg87-Pg1-Ps74 | Cg87-Pg1-Ps75 | Cg87-Pg1-Ps76 |
| Cg87-Pg1-Ps77 | Cg87-Pg1-Ps78 | Cg87-Pg1-Ps79 | Cg87-Pg1-Ps80 |
| Cg87-Pg1-Ps81 | Cg87-Pg1-Ps82 | Cg87-Pg1-Ps83 | Cg87-Pg1-Ps84 |
| Cg87-Pg1-Ps85 | Cg87-Pg1-Ps86 | Cg87-Pg1-Ps87 | Cg87-Pg1-Ps88 |
| Cg87-Pg1-Ps89 | Cg87-Pg1-Ps90 | Cg87-Pg1-Ps91 | Cg87-Pg1-Ps92 |
| Cg87-Pg1-Ps93 | Cg87-Pg1-Ps94 | Cg87-Pg1-Ps95 | Cg87-Pg1-Ps96 |
| Cg87-Pg1-Ps97 | Cg87-Pg1-Ps98 | Cg87-Pg1-Ps99 | Cg87-Pg1-Ps100 |
| Cg87-Pg1-Ps101 | Cg87-Pg1-Ps102 | Cg87-Pg1-Ps103 | Cg87-Pg1-Ps104 |
| Cg87-Pg1-Ps105 | Cg87-Pg1-Ps106 | Cg87-Pg1-Ps107 | Cg87-Pg1-Ps108 |
| Cg87-Pg1-Ps109 | Cg87-Pg1-Ps110 | Cg87-Pg1-Ps111 | Cg87-Pg1-Ps112 |
| Cg87-Pg1-Ps113 | Cg87-Pg1-Ps114 | Cg87-Pg1-Ps115 | Cg87-Pg1-Ps116 |
| Cg87-Pg1-Ps117 | Cg87-Pg1-Ps118 | Cg87-Pg1-Ps119 | Cg87-Pg1-Ps120 |
| Cg87-Pg1-Ps121 | Cg87-Pg1-Ps122 | Cg87-Pg1-Ps123 | Cg87-Pg1-Ps124 |
| Cg87-Pg1-Ps125 | Cg87-Pg1-Ps126 | Cg87-Pg1-Ps127 | Cg87-Pg1-Ps128 |
| Cg87-Pg1-Ps129 | Cg87-Pg1-Ps130 | Cg87-Pg1-Ps131 | Cg87-Pg1-Ps132 |
| Cg87-Pg1-Ps133 | Cg87-Pg1-Ps134 | Cg87-Pg1-Ps135 | Cg87-Pg1-Ps136 |
| Cg87-Pg1-Ps137 | Cg87-Pg1-Ps138 | Cg87-Pg1-Ps139 | Cg87-Pg1-Ps140 |
| Cg87-Pg1-Ps141 | Cg87-Pg1-Ps142 | Cg87-Pg1-Ps143 | Cg87-Pg1-Ps144 |
| Cg87-Pg1-Ps145 | Cg87-Pg1-Ps146 | Cg87-Pg1-Ps147 | Cg87-Pg1-Ps148 |
| Cg87-Pg1-Ps149 | Cg87-Pg1-Ps150 | Cg87-Pg1-Ps151 | Cg87-Pg1-Ps152 |
| Cg87-Pg1-Ps153 | Cg87-Pg1-Ps154 | Cg87-Pg1-Ps155 | Cg87-Pg1-Ps156 |
| Cg87-Pg1-Ps157 | Cg87-Pg1-Ps158 | Cg87-Pg1-Ps159 | Cg87-Pg1-Ps160 |
| Cg87-Pg1-Ps161 | Cg87-Pg1-Ps162 | Cg87-Pg1-Ps163 | Cg87-Pg1-Ps164 |
| Cg87-Pg1-Ps165 | Cg87-Pg1-Ps166 | Cg87-Pg1-Ps167 | Cg87-Pg1-Ps168 |
| Cg87-Pg1-Ps169 | Cg87-Pg1-Ps170 | Cg87-Pg1-Ps171 | Cg87-Pg1-Ps172 |
| Cg87-Pg1-Ps173 | Cg87-Pg1-Ps174 | Cg87-Pg1-Ps175 | Cg87-Pg1-Ps176 |
| Cg87-Pg1-Ps177 | Cg87-Pg1-Ps178 | Cg87-Pg1-Ps179 | Cg87-Pg1-Ps180 |
| Cg87-Pg1-Ps181 | Cg87-Pg1-Ps182 | Cg87-Pg1-Ps183 | Cg87-Pg1-Ps184 |
| Cg87-Pg1-Ps185 | Cg87-Pg1-Ps186 | Cg87-Pg1-Ps187 | Cg87-Pg1-Ps188 |
| Cg87-Pg1-Ps189 | Cg87-Pg1-Ps190 | Cg87-Pg1-Ps191 | Cg87-Pg1-Ps192 |
| Cg87-Pg1-Ps193 | Cg87-Pg1-Ps194 | Cg87-Pg1-Ps195 | Cg87-Pg1-Ps196 |
| Cg87-Pg1-Ps197 | Cg87-Pg1-Ps198 | Cg87-Pg1-Ps199 | Cg87-Pg1-Ps200 |
| Cg87-Pg1-Ps201 | Cg87-Pg1-Ps202 | Cg87-Pg1-Ps203 | Cg87-Pg1-Ps204 |
| Cg87-Pg1-Ps205 | Cg87-Pg1-Ps206 | Cg87-Pg1-Ps207 | Cg87-Pg1-Ps208 |
| Cg87-Pg1-Ps209 | Cg87-Pg1-Ps210 | Cg87-Pg1-Ps211 | Cg87-Pg1-Ps212 |
| Cg87-Pg1-Ps213 | Cg87-Pg1-Ps214 | Cg87-Pg1-Ps215 | Cg87-Pg1-Ps216 |
| Cg87-Pg1-Ps217 | Cg87-Pg1-Ps218 | Cg87-Pg1-Ps219 | Cg87-Pg1-Ps220 |
| Cg87-Pg1-Ps221 | Cg87-Pg1-Ps222 | Cg87-Pg1-Ps223 | Cg87-Pg1-Ps224 |
| Cg87-Pg1-Ps225 | Cg87-Pg1-Ps226 | Cg87-Pg1-Ps227 | Cg87-Pg1-Ps228 |
| Cg87-Pg1-Ps229 | Cg87-Pg1-Ps230 | Cg87-Pg1-Ps231 | Cg87-Pg1-Ps232 |
| Cg87-Pg1-Ps233 | Cg87-Pg1-Ps234 | Cg87-Pg1-Ps235 | Cg87-Pg1-Ps236 |
| Cg87-Pg1-Ps237 | Cg87-Pg1-Ps238 | Cg87-Pg1-Ps239 | Cg87-Pg1-Ps240 |
| Cg87-Pg1-Ps241 | Cg87-Pg1-Ps242 | Cg87-Pg1-Ps243 | |
| Cg88-Pg1-Ps1 | Cg88-Pg1-Ps2 | Cg88-Pg1-Ps3 | Cg88-Pg1-Ps4 |
| Cg88-Pg1-Ps5 | Cg88-Pg1-Ps6 | Cg88-Pg1-Ps7 | Cg88-Pg1-Ps8 |
| Cg88-Pg1-Ps9 | Cg88-Pg1-Ps10 | Cg88-Pg1-Ps11 | Cg88-Pg1-Ps12 |
| Cg88-Pg1-Ps13 | Cg88-Pg1-Ps14 | Cg88-Pg1-Ps15 | Cg88-Pg1-Ps16 |
| Cg88-Pg1-Ps17 | Cg88-Pg1-Ps18 | Cg88-Pg1-Ps19 | Cg88-Pg1-Ps20 |
| Cg88-Pg1-Ps21 | Cg88-Pg1-Ps22 | Cg88-Pg1-Ps23 | Cg88-Pg1-Ps24 |
| Cg88-Pg1-Ps25 | Cg88-Pg1-Ps26 | Cg88-Pg1-Ps27 | Cg88-Pg1-Ps28 |
| Cg88-Pg1-Ps29 | Cg88-Pg1-Ps30 | Cg88-Pg1-Ps31 | Cg88-Pg1-Ps32 |
| Cg88-Pg1-Ps33 | Cg88-Pg1-Ps34 | Cg88-Pg1-Ps35 | Cg88-Pg1-Ps36 |
| Cg88-Pg1-Ps37 | Cg88-Pg1-Ps38 | Cg88-Pg1-Ps39 | Cg88-Pg1-Ps40 |
| Cg88-Pg1-Ps41 | Cg88-Pg1-Ps42 | Cg88-Pg1-Ps43 | Cg88-Pg1-Ps44 |
| Cg88-Pg1-Ps45 | Cg88-Pg1-Ps46 | Cg88-Pg1-Ps47 | Cg88-Pg1-Ps48 |
| Cg88-Pg1-Ps49 | Cg88-Pg1-Ps50 | Cg88-Pg1-Ps51 | Cg88-Pg1-Ps52 |
| Cg88-Pg1-Ps53 | Cg88-Pg1-Ps54 | Cg88-Pg1-Ps55 | Cg88-Pg1-Ps56 |
| Cg88-Pg1-Ps57 | Cg88-Pg1-Ps58 | Cg88-Pg1-Ps59 | Cg88-Pg1-Ps60 |
| Cg88-Pg1-Ps61 | Cg88-Pg1-Ps62 | Cg88-Pg1-Ps63 | Cg88-Pg1-Ps64 |
| Cg88-Pg1-Ps65 | Cg88-Pg1-Ps66 | Cg88-Pg1-Ps67 | Cg88-Pg1-Ps68 |
| Cg88-Pg1-Ps69 | Cg88-Pg1-Ps70 | Cg88-Pg1-Ps71 | Cg88-Pg1-Ps72 |
| Cg88-Pg1-Ps73 | Cg88-Pg1-Ps74 | Cg88-Pg1-Ps75 | Cg88-Pg1-Ps76 |
| Cg88-Pg1-Ps77 | Cg88-Pg1-Ps78 | Cg88-Pg1-Ps79 | Cg88-Pg1-Ps80 |
| Cg88-Pg1-Ps81 | Cg88-Pg1-Ps82 | Cg88-Pg1-Ps83 | Cg88-Pg1-Ps84 |
| Cg88-Pg1-Ps85 | Cg88-Pg1-Ps86 | Cg88-Pg1-Ps87 | Cg88-Pg1-Ps88 |
| Cg88-Pg1-Ps89 | Cg88-Pg1-Ps90 | Cg88-Pg1-Ps91 | Cg88-Pg1-Ps92 |
| Cg88-Pg1-Ps93 | Cg88-Pg1-Ps94 | Cg88-Pg1-Ps95 | Cg88-Pg1-Ps96 |
| Cg88-Pg1-Ps97 | Cg88-Pg1-Ps98 | Cg88-Pg1-Ps99 | Cg88-Pg1-Ps100 |
| Cg88-Pg1-Ps101 | Cg88-Pg1-Ps102 | Cg88-Pg1-Ps103 | Cg88-Pg1-Ps104 |
| Cg88-Pg1-Ps105 | Cg88-Pg1-Ps106 | Cg88-Pg1-Ps107 | Cg88-Pg1-Ps108 |
| Cg88-Pg1-Ps109 | Cg88-Pg1-Ps110 | Cg88-Pg1-Ps111 | Cg88-Pg1-Ps112 |
| Cg88-Pg1-Ps113 | Cg88-Pg1-Ps114 | Cg88-Pg1-Ps115 | Cg88-Pg1-Ps116 |
| Cg88-Pg1-Ps117 | Cg88-Pg1-Ps118 | Cg88-Pg1-Ps119 | Cg88-Pg1-Ps120 |
| Cg88-Pg1-Ps121 | Cg88-Pg1-Ps122 | Cg88-Pg1-Ps123 | Cg88-Pg1-Ps124 |
| Cg88-Pg1-Ps125 | Cg88-Pg1-Ps126 | Cg88-Pg1-Ps127 | Cg88-Pg1-Ps128 |
| Cg88-Pg1-Ps129 | Cg88-Pg1-Ps130 | Cg88-Pg1-Ps131 | Cg88-Pg1-Ps132 |
| Cg88-Pg1-Ps133 | Cg88-Pg1-Ps134 | Cg88-Pg1-Ps135 | Cg88-Pg1-Ps136 |
| Cg88-Pg1-Ps137 | Cg88-Pg1-Ps138 | Cg88-Pg1-Ps139 | Cg88-Pg1-Ps140 |
| Cg88-Pg1-Ps141 | Cg88-Pg1-Ps142 | Cg88-Pg1-Ps143 | Cg88-Pg1-Ps144 |
| Cg88-Pg1-Ps145 | Cg88-Pg1-Ps146 | Cg88-Pg1-Ps147 | Cg88-Pg1-Ps148 |
| Cg88-Pg1-Ps149 | Cg88-Pg1-Ps150 | Cg88-Pg1-Ps151 | Cg88-Pg1-Ps152 |
| Cg88-Pg1-Ps153 | Cg88-Pg1-Ps154 | Cg88-Pg1-Ps155 | Cg88-Pg1-Ps156 |
| Cg88-Pg1-Ps157 | Cg88-Pg1-Ps158 | Cg88-Pg1-Ps159 | Cg88-Pg1-Ps160 |
| Cg88-Pg1-Ps161 | Cg88-Pg1-Ps162 | Cg88-Pg1-Ps163 | Cg88-Pg1-Ps164 |
| Cg88-Pg1-Ps165 | Cg88-Pg1-Ps166 | Cg88-Pg1-Ps167 | Cg88-Pg1-Ps168 |
| Cg88-Pg1-Ps169 | Cg88-Pg1-Ps170 | Cg88-Pg1-Ps171 | Cg88-Pg1-Ps172 |
| Cg88-Pg1-Ps173 | Cg88-Pg1-Ps174 | Cg88-Pg1-Ps175 | Cg88-Pg1-Ps176 |
| Cg88-Pg1-Ps177 | Cg88-Pg1-Ps178 | Cg88-Pg1-Ps179 | Cg88-Pg1-Ps180 |
| Cg88-Pg1-Ps181 | Cg88-Pg1-Ps182 | Cg88-Pg1-Ps183 | Cg88-Pg1-Ps184 |
| Cg88-Pg1-Ps185 | Cg88-Pg1-Ps186 | Cg88-Pg1-Ps187 | Cg88-Pg1-Ps188 |
| Cg88-Pg1-Ps189 | Cg88-Pg1-Ps190 | Cg88-Pg1-Ps191 | Cg88-Pg1-Ps192 |
| Cg88-Pg1-Ps193 | Cg88-Pg1-Ps194 | Cg88-Pg1-Ps195 | Cg88-Pg1-Ps196 |
| Cg88-Pg1-Ps197 | Cg88-Pg1-Ps198 | Cg88-Pg1-Ps199 | Cg88-Pg1-Ps200 |
| Cg88-Pg1-Ps201 | Cg88-Pg1-Ps202 | Cg88-Pg1-Ps203 | Cg88-Pg1-Ps204 |
| Cg88-Pg1-Ps205 | Cg88-Pg1-Ps206 | Cg88-Pg1-Ps207 | Cg88-Pg1-Ps208 |
| Cg88-Pg1-Ps209 | Cg88-Pg1-Ps210 | Cg88-Pg1-Ps211 | Cg88-Pg1-Ps212 |
| Cg88-Pg1-Ps213 | Cg88-Pg1-Ps214 | Cg88-Pg1-Ps215 | Cg88-Pg1-Ps216 |
| Cg88-Pg1-Ps217 | Cg88-Pg1-Ps218 | Cg88-Pg1-Ps219 | Cg88-Pg1-Ps220 |
| Cg88-Pg1-Ps221 | Cg88-Pg1-Ps222 | Cg88-Pg1-Ps223 | Cg88-Pg1-Ps224 |
| Cg88-Pg1-Ps225 | Cg88-Pg1-Ps226 | Cg88-Pg1-Ps227 | Cg88-Pg1-Ps228 |
| Cg88-Pg1-Ps229 | Cg88-Pg1-Ps230 | Cg88-Pg1-Ps231 | Cg88-Pg1-Ps232 |
| Cg88-Pg1-Ps233 | Cg88-Pg1-Ps234 | Cg88-Pg1-Ps235 | Cg88-Pg1-Ps236 |
| Cg88-Pg1-Ps237 | Cg88-Pg1-Ps238 | Cg88-Pg1-Ps239 | Cg88-Pg1-Ps240 |
| Cg88-Pg1-Ps241 | Cg88-Pg1-Ps242 | Cg88-Pg1-Ps243 | |

Abbreviations and symbols commonly used in the peptide and chemical arts are used herein to describe compounds of the present invention, following the general guidelines presented by the IUPAC-IUB Joint Commission on Biochemical Nomenclature as described in Eur. J. Biochem., 158, 9-, 1984. Compounds of formula (I) and the intermediates and starting materials used in their preparation are named in accordance with the IUPAC rules of nomenclature in which the characteristic groups have decreasing priority for citation as the principle group. An example compound of formula (I), compound (1) in which Z is CH₂, R¹ is R²C(O), where R² is 2-pyridyl, P₁, P₂ are methylene, Y is 4-methylpentoyl, (X)₀ is zero, (W)ₙ is NH, (V)ₘ is C(O) and U is phenyl is thus named:— 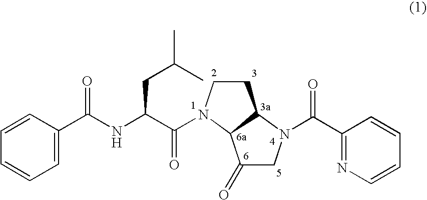
(3aR,6aS)—N-{(1S)-3-Methyl-1-[6-oxo-4-(pyridine-2-carbonyl)-hexahydropyrrolo [3,2-b]pyrrole-1-carbonyl]-butyl}-benzamide;

A second example compound of formula (I), compound (2) in which Z is CH₂, R¹ is R²C(O), where R² is 2-pyridyl, P₁, is methylene, P₂ is NH, Y is 4-methylpentoyl, (X)₀ is zero, (W)ₙ is NH, (V)ₘ is C(O) and U is phenyl is thus named:— 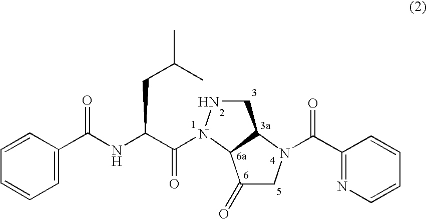
(3aR,6aS)—N-{(1S)-3-Methyl-1-[6-oxo-4-(pyridine-2-carbonyl)-hexahydropyrrolo [3,2-c]pyrazole-1-carbonyl]-butyl}-benzamide;

A third example compound of formula (I), compound (3) in which Z is CH₂, R¹ is R²C(O), where R² is 2-pyridyl, P₁, is methylene, P₂ is O, Y is 4-methylpentoyl, (X)₀ is zero, (W)ₙ is NH, (V)ₘ is C(O) and U is phenyl is thus named:— 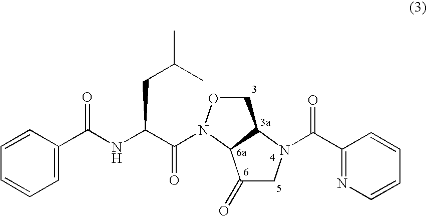
(3aS,6aS)—N-{(1S)-3-Methyl-1-[6-oxo-4-(pyridine-2-carbonyl)-hexahydro-2-oxa-1,4-diaza-pentalene-1-carbonyl]-butyl}-benzamide.

Compounds of the invention include, but are not limited to, the following examples where all 4 stereoisomeric combinations of the bicyclic ketone are included where P₂ is CH₂, i.e. (3aS,6aS), (3aR,6aS), (3aS,6aR), (3aR,6aR) and also included are the equivalent analogues where P₂ is O and NH. More preferred compounds consist of the cis-bicyclic isomers which, when P₂ is CH₂, are designated as (3aR,6aS) and (3aS,6aR) and also more preferred are the equivalent cis-bicyclic analogues where P₂ is O and NH.
4. Naphthalene-2-carboxylic acid {3-methyl-1-[6-oxo-4-(pyridine-2-sulfonyl)hexahydro-pyrrolo[3,2-b]pyrrole-1-carbonyl]-butyl}-amide;
5. Quinoline-2-carboxylic acid {3-methyl-1-[6-oxo-4-(pyridine-2-sulfonyl)-hexahydro-pyrrolo[3,2-b]pyrrole-1-carbonyl]-butyl}-amide;
6. Benzo[b]thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
7. Benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
8. Biphenyl-4-carboxylic acid {3-methyl-1-[6-oxo-4-(pyridine-2-sulfonyl)-hexahydro-pyrrolo[3,2-b]pyrrole-1-carbonyl]-butyl}-amide;
9. 4-tert-Butyl-N-{3-methyl-1-[6-oxo-4-(pyridine-2-sulfonyl)-hexahydro-pyrrolo[3,2-b]pyrrole-1-carbonyl]-butyl}-benzamide;
10. 4-Dimethylamino-N-{3-methyl-1-[6-oxo-4-(pyridine-2-sulfonyl)-hexahydro-pyrrolo[3,2-b]pyrrole-1-carbonyl]-butyl}-benzamide;
11. 7-Methoxy-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
12. {3-Methyl-1-[6-oxo-4-(pyridine-2-sulfonyl)-hexahydro-pyrrolo[3,2-b]pyrrole-1-carbonyl]-butyl}-carbamic acid benzyl ester,
13. 5-Methoxy-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
14. Thieno[3,2-b]thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
15. 3-Methyl-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
16. Quinoxaline-2-carboxylic acid {3-methyl-1-[6-oxo-4-(pyridine-2-sulfonyl)-hexahydro-pyrrolo[3,2-b]pyrrole-1-carbonyl]-butyl}-amide;
17. Benzo[1,3]dioxole-5-carboxylic acid {3-methyl-1-[6-oxo-4-(pyridine-2-sulfonyl)-hexahydro-pyrrolo[3,2-b]pyrrole-1-carbonyl]-butyl}-amide;
18. Quinoline-6-carboxylic acid {3-methyl-1-[6-oxo-4-(pyridine-2-sulfonyl)-hexahydro-pyrrolo[3,2-b]pyrrole-1-carbonyl]-butyl}-amide;
19. Furan-2-carboxylic acid {3-methyl-1-[6-oxo-4-(pyridine-2-sulfonyl)-hexahydro-pyrrolo[3,2-b]pyrrole-1-carbonyl]-butyl}-amide;
20. Thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
21. N-{3-Methyl-1-[6-oxo-4-(pyridine-2-sulfonyl)-hexahydro-pyrrolo[3,2-b]pyrrole-1-carbonyl]-butyl}-4-trifluoromethyl-benzamide;
22. Furan-3-carboxylic acid {3-methyl-1-[6-oxo-4-(pyridine-2-sulfonyl)-hexahydro-pyrrolo[3,2-b]pyrrole-1-carbonyl]-butyl}-amide;
23. Thiophene-3-carboxylic acid {3-methyl-1-[6-oxo-4-(pyridine-2-sulfonyl)-hexahydro-pyrrolo[3,2-b]pyrrole-1-carbonyl]-butyl}-amide;
24. N-{3-Methyl-1-[6-oxo-4-(pyridine-2-sulfonyl)-hexahydro-pyrrolo[3,2-b]pyrrole-1-carbonyl]-butyl}-4-phenoxy-benzamide;
25. N-{3-Methyl-1-[6-oxo-4-(pyridine-2-sulfonyl)-hexahydro-pyrrolo[3,2-b]pyrrole-1-carbonyl]-butyl}-4-(4-methyl-piperazin-1-yl)-benzamide;
26. 4-Methyl-N-{3-methyl-1-[6-oxo-4-(pyridine-2-sulfonyl)-hexahydro-pyrrolo[3,2-b]pyrrole-1-carbonyl]-butyl}-benzamide;
27. 4-Methoxy-N-{3-methyl-1-[6-oxo-4-(pyridine-2-sulfonyl)-hexahydro-pyrrolo[3,2-b]pyrrole-1-carbonyl]-butyl}-benzamide;
28. 4-Isopropyl-N-{3-methyl-1-[6-oxo-4-(pyridine-2-sulfonyl)-hexahydro-pyrrolo[3,2-b]pyrrole-1-carbonyl]-butyl}-benzamide;
29. N-{3-Methyl-1-[6-oxo-4-(pyridine-2-sulfonyl)-hexahydro-pyrrolo[3,2-b]pyrrole-1-carbonyl]-butyl}-4-vinyl-benzamide;
30. 4-Imidazol-1-yl-N-{3-methyl-1-[6-oxo-4-(pyridine-2-sulfonyl)-hexahydro-pyrrolo[3,2-b]pyrrole-1-carbonyl]-butyl}-benzamide;
31. N-{3-Methyl-1-[6-oxo-4-(pyridine-2-sulfonyl)-hexahydro-pyrrolo[3,2-b]pyrrole-1-carbonyl]-butyl}-4-thiophen-2-yl-benzamide;
32. N-{3-Methyl-1-[6-oxo-4-(pyridine-2-sulfonyl)-hexahydro-pyrrolo[3,2-b]pyrrole-1-carbonyl]-butyl}-4-oxazol-5-yl-benzamide;
33. 5-Phenyl-thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
34. N-{3-Methyl-1-[6-oxo-4-(pyridine-2-sulfonyl)hexahydro-pyrrolo[3,2-b]pyrrole-1-carbonyl]-butyl}-4-trifluoromethoxy-benzamide;
35. 5-Pyridin-2-yl-thiophene-2-carboxylic acid {3-methyl-1-[6-oxo-4-(pyridine-2-sulfonyl)hexahydro-pyrrolo[3,2-b]pyrrole-1-carbonyl]-butyl}-amide;
36. 4-Difluoromethoxy-N-{3-methyl-1-[6-oxo-4-(pyridine-2-sulfonyl)-hexahydro-pyrrolo[3,2-b]pyrrole-1-carbonyl]-butyl}-benzamide;
37. N-{3-Methyl-1-[6-oxo-4-(pyridine-2-sulfonyl)-hexahydro-pyrrolo[3,2-b]pyrrole-1-carbonyl]-butyl}-4-morpholin-4-yl-benzamide;
38. Naphthalene-2-carboxylic acid {1-[6-oxo-4-(pyridine-2-sulfonyl)hexahydro-pyrrolo[3,2-b]pyrrole-1-carbonyl]-butyl}-amide;
39. Quinoline-2-carboxylic acid {1-[6-oxo-4-(pyridine-2-sulfonyl)-hexahydro-pyrrolo[3,2-b]pyrrole-1-carbonyl]-butyl}-amide;
40. Benzo[b]thiophene-2-carboxylic acid {1-[6-oxo-4-(pyridine-2-sulfonyl)-hexahydro-pyrrolo[3,2-b]pyrrole-1-carbonyl]-butyl}-amide;
41. Benzofuran-2-carboxylic acid {1-[6-oxo-4-(pyridine-2-sulfonyl)-hexahydro-pyrrolo[3,2-b]pyrrole-1-carbonyl]-butyl}-amide;
42. Biphenyl-4-carboxylic acid {1-[6-oxo-4-(pyridine-2-sulfonyl)-hexahydro-pyrrolo[3,2-b]pyrrole-1-carbonyl]-butyl}-amide;
43. 4-tert-Butyl-N-{1-[6-oxo-4-(pyridine-2-sulfonyl)-hexahydro-pyrrolo[3,2-b]pyrrole-1-carbonyl]-butyl}-benzamide;
44. 4-Dimethylamino-N-{1-[6-oxo-4-(pyridine-2-sulfonyl)-hexahydro-pyrrolo[3,2-b]pyrrole-1-carbonyl]-butyl}-benzamide;
45. 7-Methoxy-benzofuran-2-caboxylic acid {1-[6-oxo-4-(pyridine-2-sulfonyl)-hexahydro-pyrrolo[3,2-b]pyrrole-1-carbonyl]-butyl}-amide;
46. {1-[6-oxo-4-(pyridine-2-sulfonyl)-hexahydro-pyrrolo[3,2-b]pyrrole-1-carbonyl]-butyl}-carbamic acid benzyl ester,
47. 5-Methoxy-benzofuran-2-carboxylic acid {1-[6-oxo-4-(pyridine-2-sulfonyl)-hexahydro-pyrrolo[3,2-b]pyrrole-1-carbonyl]-butyl}-amide;
48. Thieno[3,2-b]thiophene-2-carboxylic acid {1-[6-oxo-4-(pyridine-2-sulfonyl)-hexahydro-pyrrolo[3,2-b]pyrrole-1-carbonyl]-butyl}-amide;
49. 3-Methyl-benzofuran-2-carboxylic acid {1-[6-oxo-4-(pyridine-2-sulfonyl)-hexahydro-pyrrolo[3,2-b]pyrrole-1-carbonyl]-butyl}-amide;
50. Quinoxaline-2-carboxylic acid {1-[6-oxo-4-(pyridine-2-sulfonyl)-hexahydro-pyrrolo[3,2-b]pyrrole-1-carbonyl]-butyl}-amide;
51. Benzo[1,3]dioxole-5-carboxylic acid {1-[6-oxo-4-(pyridine-2-sulfonyl)-hexahydro-pyrrolo[3,2-b]pyrrole-1-carbonyl]-butyl}-amide;
52. Quinoline-6-carboxylic acid {1-[6-oxo-4-(pyridine-2-sulfonyl)-hexahydro-pyrrolo[3,2-b]pyrrole-1-carbonyl]-butyl}-amide;
53. Furan-2-carboxylic acid {1-[6-oxo-4-(pyridine-2-sulfonyl)hexahydro-pyrrolo[3,2-b]pyrrole-1-carbonyl]-butyl}-amide;
54. Thiophene-2-carboxylic acid {1-[6-oxo-4-(pyridine-2-sulfonyl)-hexahydro-pyrrolo[3,2-b]pyrrole-1-carbonyl]-butyl}-amide;
55. N-{1-[6-oxo-4-(pyridine-2-sulfonyl)-hexahydro-pyrrolo[3,2-b]pyrrole-1-carbonyl]-butyl}-4-trifluoromethyl-benzamide;
56. Furan-3-carboxylic acid {1-[6-oxo-4-(pyridine-2-sulfonyl)-hexahydro-pyrrolo[3,2-b]pyrrole-1-carbonyl]-butyl}-amide;
57. Thiophene-3-carboxylic acid {1-[6-oxo-4-(pyridine-2-sulfonyl)-hexahydro-pyrrolo[3,2-b]pyrrole-1-carbonyl]-butyl}-amide;
58. N-{1-[6-oxo-4-(pyridine-2-sulfonyl)-hexahydro-pyrrolo[3,2-b]pyrrole-1-carbonyl]-butyl}-4-phenoxy-benzamide;
59. N-{1-[6-oxo-4-(pyridine-2-sulfonyl)-hexahydro-pyrrolo[3,2-b]pyrrole-1-carbonyl]-butyl}-4-(4-methyl-piperazin-1-yl)-benzamide;
60. 4-Methyl-N-{1-[6-oxo-4-(pyridine-2-sulfonyl)-hexahydro-pyrrolo[3,2-b]pyrrole-1-carbonyl]-butyl}-benzamide;
61. 4-Methoxy-N-{1-[6-oxo-4-(pyridine-2-sulfonyl)-hexahydro-pyrrolo[3,2-b]pyrrole-1-carbonyl]-butyl}-benzamide;
62. 4-Isopropyl-N-{1-[6-oxo-4-(pyridine-2-sulfonyl)-hexahydro-pyrrolo[3,2-b]pyrrole-1-carbonyl]-butyl}-benzamide;
63. N-{1-[6-oxo-4-(pyridine-2-sulfonyl)-hexahydro-pyrrolo[3,2-b]pyrrole-1-carbonyl]-butyl}-4-vinyl-benzamide;
64. 4-Imidazol-1-yl-N-{1-[6-oxo-4-(pyridine-2-sulfonyl)-hexahydro-pyrrolo[3,2-b]pyrrole-1-carbonyl]-butyl}-benzamide;
65. N-{1-[6-oxo-4-(pyridine-2-sulfonyl)-hexahydro-pyrrolo[3,2-b]pyrrole-1-carbonyl]-butyl}-4-thiophen-2-yl-benzamide;
66. N-{1-[6-oxo-4-(pyridine-2-sulfonyl)-hexahydro-pyrrolo[3,2-b]pyrrole-1-carbonyl]-butyl}-4-oxazol-5-yl-benzamide;
67. 5-Phenyl-thiophene-2-carboxylic acid {1-[6-oxo-4-(pyridine-2-sulfonyl)-hexahydro-pyrrolo[3,2-b]pyrrole-1-carbonyl]-butyl}-amide;
68. N-{1-[6-oxo-4-(pyridine-2-sulfonyl)-hexahydro-pyrrolo[3,2-b]pyrrole-1-carbonyl]-butyl}-4-trifluoromethoxy-benzamide;
69. 5-Pyridin-2-yl-thiophene-2-carboxylic acid {1-[6-oxo-4-(pyridine-2-sulfonyl)-hexahydro-pyrrolo[3,2-b]pyrrole-1-carbonyl]-butyl}-amide;
70. 4-Difluoromethoxy-N-{1-[6-oxo-4-(pyridine-2-sulfonyl)-hexahydro-pyrrolo[3,2-b]pyrrole-1-carbonyl]-butyl}-benzamide;
71. N-{1-[6-oxo-4-(pyridine-2-sulfonyl)-hexahydro-pyrrolo[3,2-b]pyrrole-1-carbonyl]-butyl}-4-morpholin-4-yl-benzamide;
72. Naphthalene-2-carboxylic acid {3-methyl-1-[6-oxo-4-(1-oxy-pyridine-2-sulfonyl)hexahydro-pyrrolo[3,2-b]pyrrole-1-carbonyl]-butyl}-amide;
73. Quinoline-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
74. Benzo[b]thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
75. Benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
76. Biphenyl-4-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
77. 4-tert-Butyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
78. 4-Dimethylamino-N-{3-methyl-1-[6-oxo-4-(1-oxy-pyridine-2-sulfonyl)-hexahydro-pyrrolo[3,2-b]pyrrole-1-carbonyl]-butyl}-benzamide;
79. 7-Methoxy-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
80. {3-Methyl-1-[6-oxo-4-(1-oxy-pyridine-2-sulfonyl)-hexahydro-pyrrolo[3,2-b]pyrrole-1-carbonyl]-butyl}-carbamic acid benzyl ester,
81. 5-Methoxy-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
82. Thieno[3,2-b]thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
83. 3-Methyl-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
84. Quinoxaline-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
85. Benzo[1,3]dioxole-5-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
86. Quinoline-6-carboxylic acid {3-methyl-1-[6-oxo-4-(1-oxy-pyridine-2-sulfonyl)hexahydro-pyrrolo[3,2-b]pyrrole-1-carbonyl]-butyl}-amide;
87. Furan-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
88. Thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
89. N-{3-Methyl-1-[6-oxo-4-(1-oxy-pyridine-2-sulfonyl)-hexahydro-pyrrolo[3,2-b]pyrrole-1-carbonyl]-butyl}-4-trifluoromethyl-benzamide;
90. Furan-3-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
91. Thiophene-3-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
92. N-{3-Methyl-1-[6-oxo-4-(1-oxy-pyridine-2-sulfonyl)-hexahydro-pyrrolo[3,2-b]pyrrole-1-carbonyl]-butyl}-4-phenoxy-benzamide;
93. N-{3-Methyl-1-[6-oxo-4-(1-oxy-pyridine-2-sulfonyl)-hexahydro-pyrrolo[3,2-b]pyrrole-1-carbonyl]-butyl}-4-(4-methyl-piperazin-1-yl)-benzamide;
94. 4-Methyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
95. 4-Methoxy-N-{3-methyl-1-[6-oxo-4-(1-oxy-pyridine-2-sulfonyl)-hexahydro-pyrrolo[3,2-b]pyrrole-1-carbonyl]-butyl}-benzamide;
96. 4-Isopropyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
97. N-{3-Methyl-1-[6-oxo-4-(1-oxy-pyridine-2-sulfonyl)-hexahydro-pyrrolo[3,2-b]pyrrole-1-carbonyl]-butyl}-4-vinyl-benzamide;
98. 4-Imidazol-1-yl-N-{3-methyl-1-[6-oxo-4-(1-oxy-pyridine-2-sulfonyl) hexahydro-pyrrolo[3,2-b]pyrrole-1-carbonyl]-butyl}-benzamide;
99. N-{3-Methyl-1-[6-oxo-4-(1-oxy-pyridine-2-sulfonyl)-hexahydro-pyrrolo[3,2-b]pyrrole-1-carbonyl]-butyl}-4-thiophen-2-yl-benzamide;
100. N-{3-Methyl-1-[6-oxo-4-(1-oxy-pyridine-2-sulfonyl)-hexahydro-pyrrolo[3,2-b]pyrrole-1-carbonyl]-butyl}-4-oxazol-5-yl-benzamide;
101. 5-Phenyl-thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
102. N-{3-Methyl-1-[6-oxo-4-(1-oxy-pyridine-2-sulfonyl)-hexahydro-pyrrolo[3,2-b]pyrrole-1-carbonyl]-butyl}-4-trifluoromethoxy-benzamide;
103. 5-Pyridin-2-yl-thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
104. 4-Difluoromethoxy-N-{3-methyl-1-[6-oxo-4-(1-oxy-pyridine-2-sulfonyl)-hexahydro-pyrrolo[3,2-b]pyrrole-1-carbonyl]-butyl}-benzamide;
105. N-{3-Methyl-1-[6-oxo-4-(1-oxy-pyridine-2-sulfonyl)-hexahydro-pyrrolo[3,2-b]pyrrole-1-carbonyl]-butyl}-4-morpholin-4-yl-benzamide;
106. Naphthalene-2-carboxylic acid {1-[6-oxo-4-(1-oxy-pyridine-2-sulfonyl)hexahydro-pyrrolo[3,2-b]pyrrole-1-carbonyl]-butyl}-amide;
107. Quinoline-2-carboxylic acid {1-[6-oxo-4-(1-oxy-pyridine-2-sulfonyl)-hexahydro-pyrrolo[3,2-b]pyrrole-1-carbonyl]-butyl}-amide;
108. Benzo[b]thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
109. Benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
110. Biphenyl-4-carboxylic acid {1-[6-oxo-4-(1-oxy-pyridine-2-sulfonyl)-hexahydro-pyrrolo[3,2-b]pyrrole-1-carbonyl]-butyl}-amide;
111. 4-tert-Butyl-N-{1-[6-oxo-4-(1-oxy-pyridine-2-sulfonyl)-hexahydro-pyrrolo[3,2-b]pyrrole-1-carbonyl]-butyl}-benzamide;
112. 4-Dimethylamino-N-{-1-[6-oxo-4-(1-oxy-pyridine-2-sulfonyl)-hexahydro-pyrrolo[3,2-b]pyrrole-1-carbonyl]-butyl}-benzamide;
113. 7-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
114. {1-[6-oxo-4-(1-oxy-pyridine-2-sulfonyl)hexahydro-pyrrolo[3,2-b]pyrrole-1-carbonyl]-butyl}-carbamic acid benzyl ester,
115. 5-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
116. Thieno[3,2-b]thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
117. 3-Methyl-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
118. Quinoxaline-2-carboxylic acid {1-[6-oxo-4-(1-oxy-pyridine-2-sulfonyl)-hexahydro-pyrrolo[3,2-b]pyrrole-1-carbonyl]-butyl}-amide;
119. Benzo[1,3]dioxole-5-carboxylic acid {1-[6-oxo-4-(1-oxy-pyridine-2-sulfonyl)-hexahydro-pyrrolo[3,2-b]pyrrole-1-carbonyl]-butyl}-amide;
120. Quinoline-6-carboxylic acid {1-[6-oxo-4-(1-oxy-pyridine-2-sulfonyl)-hexahydro-pyrrolo[3,2-b]pyrrole-1-carbonyl]-butyl}-amide;
121. Furan-2-carboxylic acid {1-[6-oxo-4-(1-oxy-pyridine-2-sulfonyl)-hexahydro-pyrrolo[3,2-b]pyrrole-1-carbonyl]-butyl}-amide;
122. Thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
123. N-{1-[6-oxo-4-(1-oxy-pyridine-2-sulfonyl)-hexahydro-pyrrolo[3,2-b]pyrrole-1-carbonyl]-butyl}-4-trifluoromethyl-benzamide;
124. Furan-3-carboxylic acid {1-[6-oxo-4-(1-oxy-pyridine-2-sulfonyl)-hexahydro-pyrrolo[3,2-b]pyrrole-1-carbonyl]-butyl}-amide;
125. Thiophene-3-carboxylic acid {1-[6-oxo-4-(1-oxy-pyridine-2-sulfonyl)-hexahydro-pyrrolo[3,2-b]pyrrole-1-carbonyl]-butyl}-amide;
126. N-{1-[6-oxo-4-(1-oxy-pyridine-2-sulfonyl)-hexahydro-pyrrolo[3,2-b]pyrrole-1-carbonyl]-butyl}-4-phenoxy-benzamide;
127. N-{1-[6-oxo-4-(1-oxy-pyridine-2-sulfonyl)-hexahydro-pyrrolo[3,2-b]pyrrole-1-carbonyl]-butyl}-4-(4-methyl-piperazin-1-yl)benzamide;
128. 4-Methyl-N-{1-[6-oxo-4-(1-oxy-pyridine-2-sulfonyl)-hexahydro-pyrrolo[3,2-b]pyrrole-1-carbonyl]-butyl}-benzamide;
129. 4-Methoxy-N-{1-[6-oxo-4-(1-oxy-pyridine-2-sulfonyl)-hexahydro-pyrrolo[3,2-b]pyrrole-1-carbonyl]-butyl}-benzamide;
130. 4-Isopropyl-N-{1-[6-oxo-4-(1-oxy-pyridine-2-sulfonyl)hexahydro-pyrrolo[3,2-b]pyrrole-1-carbonyl]-butyl}-benzamide;
131. N-{1-[6-oxo-4-(1-oxy-pyridine-2-sulfonyl)-hexahydro-pyrrolo[3,2-b]pyrrole-1-carbonyl]-butyl}-4-vinyl-benzamide;
132. 4-Imidazol-1-yl-N-{1-[6-oxo-4-(1-oxy-pyridine-2-sulfonyl)-hexahydro-pyrrolo[3,2-b]pyrrole-1-carbonyl]-butyl}-benzamide;
133. N-{1-[6-oxo-4-(1-oxy-pyridine-2-sulfonyl)-hexahydro-pyrrolo[3,2-b]pyrrole-1-carbonyl]-butyl}-4-thiophen-2-yl-benzamide;
134. N-{1-[6-oxo-4-(1-oxy-pyridine-2-sulfonyl)-hexahydro-pyrrolo[3,2-b]pyrrole-1-carbonyl]-butyl}-4-oxazol-5-yl-benzamide;
135. 5-Phenyl-thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
136. N-{1-[6-oxo-4-(1-oxy-pyridine-2-sulfonyl)-hexahydro-pyrrolo[3,2-b]pyrrole-1-carbonyl]-butyl}-4-trifluoromethoxy-benzamide;
137. 5-Pyridin-2-yl-thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
138. 4-Difluoromethoxy-N-{1-[6-oxo-4-(1-oxy-pyridine-2-sulfonyl)-hexahydro-pyrrolo[3,2-b]pyrrole-1-carbonyl]-butyl}-benzamide;
139. N-{1-[6-oxo-4-(1-oxy-pyridine-2-sulfonyl)-hexahydro-pyrrolo[3,2-b]pyrrole-1-carbonyl]-butyl}-4-morpholin-4-yl-benzamide;
140. Naphthalene-2-carboxylic acid {3-methyl-1-[6-oxo-4-(pyridine-3-sulfonyl)hexahydro-pyrrolo[3,2-b]pyrrole-1-carbonyl]-butyl}-amide;
141. Quinoline-2-carboxylic acid {3-methyl-1-[6-oxo-4-(pyridine-3-sulfonyl)-hexahydro-pyrrolo[3,2-b]pyrrole-1-carbonyl]-butyl}-amide;
142. Benzo[b]thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
143. Benzofuran-2-caboxylic acid {3-methyl-1-[6-oxo-4-(pyridine-3-sulfonyl)hexahydro-pyrrolo[3,2-b]pyrrole-1-carbonyl]-butyl}-amide;
144. Biphenyl-4-carboxylic acid {3-methyl-1-[6-oxo-4-(pyridine-3-sulfonyl)-hexahydro-pyrrolo[3,2-b]pyrrole-1-carbonyl]-butyl}-amide;
145. 4-tert-Butyl-N-{3-methyl-1-[6-oxo-4-(pyridine-3-sulfonyl)-hexahydro-pyrrolo[3,2-b]pyrrole-1-carbonyl]-butyl}-benzamide;
146. 4-Dimethylamino-N-{3-methyl-1-[6-oxo-4-(pyridine-3-sulfonyl)-hexahydro-pyrrolo[3,2-b]pyrrole-1-carbonyl]-butyl}-benzamide;
147. 7-Methoxy-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
148. {3-Methyl-1-[6-oxo-4-(pyridine-3-sulfonyl)-hexahydro-pyrrolo[3,2-b]pyrrole-1-carbonyl]-butyl}-carbamic acid benzyl ester;
149. 5-Methoxy-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
150. Thieno[3,2-b]thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
151. 3-Methyl-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
152. Quinoxaline-2-carboxylic acid {3-methyl-1-[6-oxo-4-(pyridine-3-sulfonyl)-hexahydro-pyrrolo[3,2-b]pyrrole-1-carbonyl]-butyl}-amide;
153. Benzo[1,3]dioxole-5-carboxylic acid {3-methyl-1-[6-oxo-4-(pyridine-3-sulfonyl)-hexahydro-pyrrolo[3,2-b]pyrrole-1-carbonyl]-butyl}-amide;
154. Quinoline-6-carboxylic acid {3-methyl-1-[6-oxo-4-(pyridine-3-sulfonyl)-hexahydro-pyrrolo[3,2-b]pyrrole-1-carbonyl]-butyl}-amide;
155. Furan-2-carboxylic acid {3-methyl-1-[6-oxo-4-(pyridine-3-sulfonyl)-hexahydro-pyrrolo[3,2-b]pyrrole-1-carbonyl]-butyl}-amide;
156. Thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
157. N-{3-Methyl-1-[6-oxo-4-(pyridine-3-sulfonyl)-hexahydro-pyrrolo[3,2-b]pyrrole-1-carbonyl]-butyl}-4-trifluoromethyl-benzamide;
158. Furan-3-carboxylic acid {3-methyl-1-[6-oxo-4-(pyridine-3-sulfonyl)-hexahydro-pyrrolo[3,2-b]pyrrole-1-carbonyl]-butyl}-amide;
159. Thiophene-3-carboxylic acid {3-methyl-1-[6-oxo-4-(pyridine-3-sulfonyl)-hexahydro-pyrrolo[3,2-b]pyrrole-1-carbonyl]-butyl}-amide;
160. N-{3-Methyl-1-[6-oxo-4-(pyridine-3-sulfonyl)-hexahydro-pyrrolo[3,2-b]pyrrole-1-carbonyl]-butyl}-4-phenoxy-benzamide;
161. N-{3-Methyl-1-[6-oxo-4-(pyridine-3-sulfonyl)-hexahydro-pyrrolo[3,2-b]pyrrole-1-carbonyl]-butyl}-4-(4-methyl-piperazin-1-yl)-benzamide;
162. 4-Methyl-N-{3-methyl-1-[6-oxo-4-(pyridine-3-sulfonyl)-hexahydro-pyrrolo[3,2-b]pyrrole-1-carbonyl]-butyl}-benzamide;
163. 4-Methoxy-N-{3-methyl-1-[6-oxo-4-(pyridine-3-sulfonyl)-hexahydro-11 pyrrolo[3,2-b]pyrrole-1-carbonyl]-butyl}-benzamide;
164. 4-Isopropyl-N-{3-methyl-1-[6-oxo-4-(pyridine-3-sulfonyl)-hexahydro-pyrrolo[3,2-b]pyrrole-1-carbonyl]-butyl}-benzamide;
165. N-{3-Methyl-1-[6-oxo-4-(pyridine-3-sulfonyl)-hexahydro-pyrrolo[3,2-b]pyrrole-1-carbonyl]-butyl}-4-vinyl-benzamide;
166. 4-imidazol-1-yl-N-{3-methyl-1-[6-oxo-4-(pyridine-3-sulfonyl)-hexahydro-pyrrolo[3,2-b]pyrrole-1-carbonyl]-butyl}-benzamide;
167. N-{3-Methyl-1-[6-oxo-4-(pyridine-3-sulfonyl)-hexahydro-pyrrolo[3,2-b]pyrrole-1-carbonyl]-butyl}-4-thiophen-2-yl-benzamide;
168. N-{3-Methyl-1-[6-oxo-4-(pyridine-3-sulfonyl)-hexahydro-pyrrolo[3,2-b]pyrrole-1-carbonyl]-butyl}-4-oxazol-5-yl-benzamide;
169. 5-Phenyl-thiophene-2 carboxylic acid {3-methyl-1-[6-oxo-4-(pyridine-3-sulfonyl)-hexahydro-pyrrolo[3,2-b]pyrrole-1-carbonyl]-butyl}-amide;
170. N-{3-Methyl-1-[6-oxo-4-(pyridine-3-sulfonyl)-hexahydro-pyrrolo[3,2-b]pyrrole-1-carbonyl]-butyl}-4-trifluoromethoxy-benzamide;
171. 5-Pyridin-2-yl-thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
172. 4-Difluoromethoxy-N-{3-methyl-1-[6-oxo-4-(pyridine-3-sulfonyl)-hexahydro-pyrrolo[3,2-b]pyrrole-1-carbonyl]-butyl}-benzamide;
173. N-{3-Methyl-1-[6-oxo-4-(pyridine-3-sulfonyl)-hexahydro-pyrrolo[3,2-b]pyrrole-1-carbonyl]-butyl}-4-morpholin-4-yl-benzamide;
174. Naphthalene-2-carboxylic acid {1-[6-oxo-4-(pyridine-3-sulfonyl)hexahydro-pyrrolo[3,2-b]pyrrole-1-carbonyl]-butyl}-amide;
175. Quinoline-2-carboxylic acid {1-[6-oxo-4-(pyridine-3-sulfonyl)-hexahydro-pyrrolo[3,2-b]pyrrole-1-carbonyl]-butyl}-amide;
176. Benzo[b]thiophene-2-carboxylic acid {1-[6-oxo-4-(pyridine-3-sulfonyl)-hexahydro-pyrrolo[3,2-b]pyrrole-1-carbonyl]-butyl}-amide;
177. Benzofuran-2-carboxylic acid {1-[6-oxo-4-(pyridine-3-sulfonyl) hexahydro-pyrrolo[3,2-b]pyrrole-1-carbonyl]-butyl}-amide;
178. Biphenyl-4-carboxylic acid {1-[6-oxo-4-(pyridine-3-sulfonyl)-hexahydro pyrrolo[3,2-b]pyrrole-1-carbonyl]-butyl}-amide;
179. 4-tert-Butyl-N-{1-[6-oxo-4-(pyridine-3-sulfonyl)-hexahydro-pyrrolo[3,2-b]pyrrole-1-carbonyl]-butyl}-benzamide;
180. 4-Dimethylamino-N-{1-[6-oxo-4-(pyridine-3-sulfonyl)-hexahydro-pyrrolo[3,2-b]pyrrole-1-carbonyl]-butyl}-benzamide;
181. 7-Methoxy-benzofuran-2-carboxylic acid {1-[6-oxo-4-(pyridine-3-sulfonyl)-hexahydro-pyrrolo[3,2-b]pyrrole-1-carbonyl]-butyl}-amide;
182. {1-[6-oxo-4-(pyridine-3-sulfonyl)-hexahydro-pyrrolo[3,2-b]pyrrole-1-carbonyl]-butyl}-carbamic acid benzyl ester;
183. 5-Methoxy-benzofuran-2-carboxylic acid {1-[6-oxo-4-(pyridine-3-sulfonyl)-hexahydro-pyrrolo[3,2-b]pyrrole-1-carbonyl]-butyl}-amide;
184. Thieno[3,2-b]thiophene-2-carboxylic acid {1-[6-oxo-4-(pyridine-3-sulfonyl)-hexahydro-pyrrolo[3,2-b]pyrrole-1-carbonyl]-butyl}-amide;
185. 3-Methyl-benzofuran-2-carboxylic acid {1-[6-oxo-4-(pyridine-3-sulfonyl) hexahydro-pyrrolo[3,2-b]pyrrole-1-carbonyl]-butyl}-amide;
186. Quinoxaline-2-carboxylic acid {1-[6-oxo-4-(pyridine-3-sulfonyl)-hexahydro-pyrrolo[3,2-b]pyrrole-1-carbonyl]-butyl}-amide;
187. Benzo[1,3]dioxole-5-carboxylic acid {1-[6-oxo-4-(pyridine-3-sulfonyl)-hexahydro-pyrrolo[3,2-b]pyrrole-1-carbonyl]-butyl}-amide;
188. Quinoline-6-carboxylic acid {1-[6-oxo-4-(pyridine-3-sulfonyl)-hexahydro-pyrrolo[3,2-b]pyrrole-1-carbonyl]-butyl}-amide;
189. Furan-2-carboxylic acid {1-[6-oxo-4-(pyridine-3-sulfonyl)-hexahydro-pyrrolo[3,2-b]pyrrole-1-carbonyl]-butyl}-amide;
190. Thiophene-2-carboxylic acid {1-[6-oxo-4-(pyridine-3-sulfonyl) hexahydro-pyrrolo[3,2-b]pyrrole-1-carbonyl]-butyl}-amide;
191. N-{1-[6-oxo-4-(pyridine-3-sulfonyl)-hexahydro-pyrrolo[3,2-b]pyrrole-1-carbonyl]-butyl}-4-trifluoromethyl-benzamide;
192. Furan-3-carboxylic acid {1-[6-oxo-4-(pyridine-3-sulfonyl)-hexahydro-pyrrolo[3,2-b]pyrrole-1-carbonyl]-butyl}-amide;
193. Thiophene-3-carboxylic acid {1-[6-oxo-4-(pyridine-3-sulfonyl)-hexahydro-pyrrolo[3,2-b]pyrrole-1-carbonyl]-butyl}-amide;
194. N-{1-[6-oxo-4-(pyridine-3-sulfonyl)-hexahydro-pyrrolo[3,2-b]pyrrole-1-carbonyl]-butyl}-4-phenoxy-benzamide;
195. N-{1-[6-oxo-4-(pyridine-3-sulfonyl)-hexahydro-pyrrolo[3,2-b]pyrrole-1-carbonyl]-butyl}-4-(methyl-piperazin-1-yl)-benzamide;
196. 4-Methyl-N-{1-[6-oxo-4-(pyridine-3-sulfonyl)-hexahydro-pyrrolo[3,2-b]pyrrole-1-carbonyl]-butyl}-benzamide;
197. 4-Methoxy-N-{1-[6-oxo-4-(pyridine-3-sulfonyl)-hexahydro-pyrrolo[3,2-b]pyrrole-1-carbonyl]-butyl}-benzamide;
198. 4-Isopropyl-N-{1-[6-oxo-4-(pyridine-3-sulfonyl)-hexahydro-pyrrolo[3,2-b]pyrrole-1-carbonyl]-butyl}-benzamide;
199. N-{1-[6-oxo-4-(pyridine-3-sulfonyl)-hexahydro-pyrrolo[3,2-b]pyrrole-1-carbonyl]-butyl}-4-vinyl-benzamide;
200. 4-Imidazol-1-yl-N-{1-[6-oxo-4-(pyridine-3-sulfonyl)-hexahydro-pyrrolo[3,2-b]pyrrole-1-carbonyl]-butyl}-benzamide;
201. N-{1-[6-oxo-4-(pyridine-3-sulfonyl)-hexahydro-pyrrolo[3,2-b]pyrrole-1-carbonyl]-butyl}-4-thiophen-2-yl-benzamide;
202. N-{1-[6-oxo-4-(pyridine-3-sulfonyl)-hexahydro-pyrrolo[3,2-b]pyrrole-1-carbonyl]-butyl}-4-oxazol-5-yl-benzamide;
203. 5-Phenyl-thiophene-2-carboxylic acid {1-[6-oxo-4-(pyridine-3-sulfonyl)-hexahydro-pyrrolo[3,2-b]pyrrole-1-carbonyl]-butyl}-amide;
204. N-{1-[6-oxo-4-(pyridine-3-sulfonyl)-hexahydro-pyrrolo[3,2-b]pyrrole-1-carbonyl]-butyl}-4-trifluoromethoxy-benzamide;
205. 5-Pyridin-2-yl-thiophene-2-carboxylic acid {1-[6-oxo-4-(pyridine-3-sulfonyl)-hexahydro-pyrrolo[3,2-b]pyrrole-1-carbonyl]-butyl}-amide;
206. 4-Difluoromethoxy-N-{1-[6-oxo-4-(pyridine-3-sulfonyl)-hexahydro-pyrrolo[3,2-b]pyrrole-1-carbonyl]-butyl}-benzamide;
207. N-{1-[6-oxo-4-(pyridine-3-sulfonyl)-hexahydro-pyrrolo[3,2-b]pyrrole-1-carbonyl]-butyl}-4-morpholin-4-yl-benzamide;
208. Naphthalene-2-carboxylic acid {3-methyl-1-[6-oxo-4-(1-oxy-pyridine-3-sulfonyl)hexahydro-pyrrolo[3,2-b]pyrrole-1-carbonyl]-butyl}-amide;
209. Quinoline-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
210. Benzo[b]thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
211. Benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
212. Biphenyl-4-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
213. 4-tert-Butyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
214. 4-Dimethylamino-N-{3-methyl-1-[6-oxo-4-(1-oxy-pyridine-3-sulfonyl)-hexahydro-pyrrolo[3,2-b]pyrrole-1-carbonyl]-butyl}-benzamide;
215. 7-Methoxy-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
216. {3-Methyl-1-[6-oxo-4-(1-oxy-pyridine-3-sulfonyl)-hexahydro-pyrrolo[3,2-b]pyrrole-1-carbonyl]-butyl}-carbamic acid benzyl ester,
217. 5-Methoxy-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
218. Thieno[3,2-b]thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
219. 3-Methyl-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
220. Quinoxaline-2-carboxylic acid {3-methyl-1-[6-oxo-4-(1-oxy-pyridine-3-sulfonyl)hexahydro-pyrrolo[3,2-b]pyrrole-1-carbonyl]-butyl}-amide;
221. Benzo[1,3]dioxole-5-carboxylic acid {3-methyl-1-[6-oxo-4-(1-oxy-pyridine-3-sulfonyl)hexahydro-pyrrolo[3,2-b]pyrrole-carbonyl]-butyl}-amide;
222. Quinoline-6-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
223. Furan-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
224. Thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
225. N-{3-Methyl-1-[6-oxo-4-(1-oxy-pyridine-3-sulfonyl)-hexahydro-pyrrolo[3,2-b]pyrrole-1-carbonyl]-butyl}-4-trifluoromethyl-benzamide;
226. Furan-3-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
227. Thiophene-3-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
228. N-{3-Methyl-1-[6-oxo-4-(1-oxy-pyridine-3-sulfonyl)-hexahydro-pyrrolo[3,2-b]pyrrole-1-carbonyl]-butyl}-4-phenoxy-benzamide;
229. N-{3-Methyl-1-[6-oxo-4-(1-oxy-pyridine-3-sulfonyl)-hexahydro-pyrrolo[3,2-b]pyrrole-1-carbonyl]-butyl}-4-(4-methyl-piperazin-1-yl)-benzamide;
230. 4-Methyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
231. 4-Methoxy-N-{3-methyl-1-[6-oxo-4-(1-oxy-pyridine-3-sulfonyl)-hexahydro-pyrrolo[3,2-b]pyrrole-1-carbonyl]-butyl}-benzamide;
232. 4-Isopropyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
233. N-{3-Methyl-1-[6-oxo-4-(1-oxy-pyridine-3-sulfonyl)-hexahydro-pyrrolo[3,2-b]pyrrole-1-carbonyl]-butyl}-4-vinyl-benzamide;
234. 4-Imidazol-1-yl-N-{3-methyl-1-[6-oxo-4-(1-oxy-pyridine-3-sulfonyl)-hexahydro-pyrrolo[3,2-b]pyrrole-1-carbonyl]-butyl}-benzamide;
235. N-{3-Methyl-1-[6-oxo-4-(1-oxy-pyridine-3-sulfonyl)-hexahydro-pyrrolo[3,2-b]pyrrole-1-carbonyl]-butyl}-4-thiophen-2-yl-benzamide;
236. N-{3-Methyl-1-[6-oxo-4-(1-oxy-pyridine-3-sulfonyl)-hexahydro-pyrrolo[3,2-b]pyrrole-1-carbonyl]-butyl}-4-oxazol-5-yl-benzamide;
237. 5-Phenyl-thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
238. N-{3-Methyl-1-[6-oxo-4-(1-oxy-pyridine-3-sulfonyl)-hexahydro-pyrrolo[3,2-b]pyrrole-1-carbonyl]-butyl}-4-trifluoromethoxy-benzamide;
239. 5-Pyridin-2-yl-thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
240. 4-Difluoromethoxy-N-{3-methyl-1-[6-oxo-4-(1-oxy-pyridine-3-sulfonyl)-hexahydro-pyrrolo[3,2-b]pyrrole-1-carbonyl]-butyl}-benzamide;
241. N-{3-Methyl-1-[6-oxo-4-(1-oxy-pyridine-3-sulfonyl)-hexahydro-pyrrolo[3,2-b]pyrrole-1-carbonyl]-butyl}-4-morpholin-4-yl-benzamide;
242. Naphthalene-2-carboxylic acid {1-[6-oxo-4-(1-oxy-pyridine-3-sulfonyl)hexahydro-pyrrolo[3,2-b]pyrrole-1-carbonyl]-butyl}-amide;
243. Quinoline-2-carboxylic acid {1-[6-oxo-4-(1-oxy-pyridine-3-sulfonyl)-hexahydro-pyrrolo[3,2-b]pyrrole-1-carbonyl]-butyl}-amide;
244. Benzo[b]thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
245. Benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
246. Biphenyl-4-carboxylic acid {1-[6-oxo-4-(1-oxy-pyridine-3-sulfonyl)-hexahydro-pyrrolo[3,2-b]pyrrole-1-carbonyl]-butyl}-amide;
247. 4-tert-Butyl-N-{1-[6-oxo-4-(1-oxy-pyridine-3-sulfonyl)-hexahydro-pyrrolo[3,2-b]pyrrole-1-carbonyl]-butyl}-benzamide;
248. 4-Dimethylamino-N-{1-[6-oxo-4-(1-oxy-pyridine-3-sulfonyl)-hexahydro-pyrrolo[3,2-b]pyrrole-1-carbonyl]-butyl}-benzamide;
249. 7-Methoxy-benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
250. {1-[6-oxo-4-(1-oxy-pyridine-3-sulfonyl)hexahydro-pyrrolo[3,2-b]pyrrole-1-carbonyl]-butyl}-carbamic acid benzyl ester,
251. 5-Methoxy-benzofuran-2-caboxylic acid {1-[6-oxo-4-(1-oxy-pyridine-3-sulfonyl)hexahydro-pyrrolo[3,2-b]pyrrole-1-carbonyl]-butyl}-amide;
252. Thieno[3,2-b]thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
253. 3-Methyl-benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
254. Quinoxaline-2-carboxylic acid {1-[6-oxo-4-(1-oxy-pyridine-3-sulfonyl)-hexahydro-pyrrolo[3,2-b]pyrrole-1-carbonyl]-butyl}-amide;
255. Benzo[1,3]dioxole-5-carboxylic acid {1-[6-oxo-4-(1-oxy-pyridine-3-sulfonyl)-hexahydro-pyrrolo[3,2-b]pyrrole-1-carbonyl]-butyl}-amide;
256. Quinoline-6-carboxylic acid {1-[6-oxo-4-(1-oxy-pyridine-3-sulfonyl)-hexahydro-pyrrolo[3,2-b]pyrrole-1-carbonyl]-butyl}-amide;
257. Furan-2-carboxylic acid {1-[6-oxo-4-(1-oxy-pyridine-3-sulfonyl)-hexahydro-pyrrolo[3,2-b]pyrrole-1-carbonyl]-butyl}-amide;
258. Thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
259. N-{1-[6-oxo-4-(1-oxy-pyridine-3-sulfonyl)-hexahydro-pyrrolo[3,2-b]pyrrole-1-carbonyl]-butyl}-4-trifluoromethyl-benzamide;
260. Furan-3-carboxylic acid {1-[6-oxo-4-(1-oxy-pyridine-3-sulfonyl)-hexahydro-pyrrolo[3,2-b]pyrrole-1-carbonyl]-butyl}-amide;
261. Thiophene-3-carboxylic acid {1-[6-oxo-4-(1-oxy-pyridine-3-sulfonyl)-hexahydro-pyrrolo[3,2-b]pyrrole-1-carbonyl]-butyl}-amide;
262. N-{1-[6-oxo-4-(1-oxy-pyridine-3-sulfonyl)-hexahydro-pyrrolo[3,2-b]pyrrole-1-carbonyl]-butyl}-4-phenoxy-benzamide;
263. N-{1-[6-oxo-4-(1-oxy-pyridine-3-sulfonyl)-hexahydro-pyrrolo[3,2-b]pyrrole-1-carbonyl]-butyl}-4-(4-methyl-piperazin-1-yl)-benzamide;
264. 4-Methyl-N-{1-[6-oxo-4-(1-oxy-pyridine-3-sulfonyl)-hexahydro-pyrrolo[3,2-b]pyrrole-1-carbonyl]-butyl}-benzamide;
265. 4-Methoxy-N-{1-[6-oxo-4-(1-oxy-pyridine-3-sulfonyl)-hexahydro-pyrrolo[3,2-b]pyrrole-1-carbonyl]-butyl}-benzamide;
266. 4-Isopropyl-N-{1-[6-oxo-4-(1-oxy-pyridine-3-sulfonyl)-hexahydro-pyrrolo[3,2-b]pyrrole-1-carbonyl]-butyl}-benzamide;
267. N-{1-[6-oxo-4-(1-oxy-pyridine-3-sulfonyl)-hexahydro-pyrrolo[3,2-b]pyrrole-1-carbonyl]-butyl}-4-vinyl-benzamide;
268. 4-Imidazol-1-yl-N-{1-[6-oxo-4-(1-oxy-pyridine-3-sulfonyl)-hexahydro-pyrrolo[3,2-b]pyrrole-1-carbonyl]-butyl}-benzamide;
269. N-f{-[6-oxo-4-(1-oxy-pyridine-3-sulfonyl)-hexahydro-pyrrolo[3,2-b]pyrrole-1-carbonyl]-butyl}-4-thiophen-2-yl-benzamide;
270. N-{1-[6-oxo-4-(1-oxy-pyridine-3-sulfonyl)-hexahydro-pyrrolo[3,2-b]pyrrole-1-carbonyl]-butyl}-4-oxazol-5-yl-benzamide;
271. 5-Phenyl-thiophene-2-carboxylic acid {1-[6-oxo-4-(1-oxy-pyridine-3-sulfonyl)hexahydro-pyrrolo[3,2-b]pyrrole-1-carbonyl]-butyl}-amide;
272. N-{1-[6-oxo-4-(1-oxy-pyridine-3-sulfonyl)-hexahydro-pyrrolo[3,2-b]pyrrole-1-carbonyl]-butyl}-4-trifluoromethoxy-benzamide;
273. 5-Pyridin-2-yl-thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
274. 4-Difluoromethoxy-N-{1-[6-oxo-4-(1-oxy-pyridine-3-sulfonyl)-hexahydro-pyrrolo[3,2-b]pyrrole-1-carbonyl]-butyl}-benzamide;
275. N-{1-[6-oxo-4-(1-oxy-pyridine-3-sulfonyl)-hexahydro-pyrrolo[3,2-b]pyrrole-1-carbonyl]-butyl}-4-morpholin-4-yl-benzamide;
276. Naphthalene-2-carboxylic acid {3-methyl-1-[6-oxo-4-(pyridine-2-carbonyl)hexahydro-pyrrolo[3,2-b]pyrrole-1-carbonyl]-butyl}-amide;
277. Quinoline-2-carboxylic acid {3-methyl-1-[6-oxo-4-(pyridine-2-carbonyl)-hexahydro-pyrrolo[3,2-b]pyrrole-1-carbonyl]-butyl}-amide;
278. Benzo[b]thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
279. Benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
280. Biphenyl-4-carboxylic acid {3-methyl-1-[6-oxo-4-(pyridine-2-carbonyl)-hexahydro-pyrrolo[3,2-b]pyrrole-1-carbonyl]-butyl}-amide;
281. 4-tert-Butyl-N-{3-methyl-1-[6-oxo-4-(pyridine-2-carbonyl)-hexahydro-pyrrolo[3,2-b]pyrrole-1-carbonyl]-butyl}-benzamide;
282. 4-Dimethylamino-N-{3-methyl-1-[6-oxo-4-(pyridine-2-carbonyl)-hexahydro-pyrrolo[3,2-b]pyrrole-1-carbonyl]-butyl}-benzamide;
283. 7-Methoxy-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
284. {3-Methyl-1-[6-oxo-4-(pyridine-2-carbonyl)-hexahydro-pyrrolo[3,2-b]pyrrole-1-carbonyl]-butyl}-carbamic acid benzyl ester;
285. 5-Methoxy-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
286. Thieno[3,2-b]thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
287. 3-Methyl-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
288. Quinoxaline-2-carboxylic acid {3-methyl-1-[6-oxo-4-(pyridine-2-carbonyl)-hexahydro-pyrrolo[3,2-b]pyrrole-1-carbonyl]-butyl}-amide;
289. Benzo[1,3]dioxole-5-carboxylic acid {3-methyl-1-[6-oxo-4-(pyridine-2-carbonyl)-hexahydro-pyrrolo[3,2-b]pyrrole-1-carbonyl]-butyl}-amide;
290. Quinoline-6-carboxylic acid {3-methyl-1-[6-oxo-4-(pyridine-2-carbonyl)-hexahydro-pyrrolo[3,2-b]pyrrole-1-carbonyl]-butyl}-amide;
291. Furan-2-carboxylic acid {3-methyl-1-[6-oxo-4-(pyridine-2-carbonyl)-hexahydro-pyrrolo[3,2-b]pyrrole-1-carbonyl]-butyl}-amide;
292. Thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
293. N-{3-Methyl-1-[6-oxo-4-(pyridine-2-carbonyl)-hexahydro-pyrrolo[3,2-b]pyrrole-1-carbonyl]-butyl}-4-trifluoromethyl-benzamide;
294. Furan-3-carboxylic acid {3-methyl-1-[6-oxo-4-(pyridine-2-carbonyl)-hexahydro-pyrrolo[3,2-b]pyrrole-1-carbonyl]-butyl}-amide;
295. Thiophene-3-carboxylic acid {3-methyl-1-[6-oxo-4-(pyridine-2-carbonyl)-hexahydro-pyrrolo[3,2-b]pyrrole-1-carbonyl]-butyl}-amide;
296. N-{3-Methyl-1-[6-oxo-4-(pyridine-2-carbonyl)-hexahydro-pyrrolo[3,2-b]pyrrole-1-carbonyl]-butyl}-4-phenoxy-benzamide;
297. N-{3-Methyl-1-[6-oxo-4-(pyridine-2-carbonyl)-hexahydro-pyrrolo[3,2-b]pyrrole-1-carbonyl]-butyl}-4-(4-methyl-piperazin-1-yl)-benzamide;
298. 4-Methyl-N-{3-methyl-1-[6-oxo-4-(pyridine-2-carbonyl)hexahydro-pyrrolo[3,2-b]pyrrole-1-carbonyl]-butyl}-benzamide;
299. 4-Methoxy-N-{3-methyl-1-[6-oxo-4-(pyridine-2-carbonyl)hexahydro-pyrrolo[3,2-b]pyrrole-1-carbonyl]-butyl}-benzamide;
300. 4-Isopropyl-N-{3-methyl-1-[6-oxo-4-(pyridine-2-carbonyl)-hexahydro-pyrrolo[3,2-b]pyrrole-1-carbonyl]-butyl}-benzamide;
301. N-{3-Methyl-1-[6-oxo-4-(pyridine-2-carbonyl)-hexahydro-pyrrolo[3,2-b]pyrrole-1-carbonyl]-butyl}-4-vinyl-benzamide;
302. 4-Imidazol-1-yl-N-{3-methyl-1-[6-oxo-4-(pyridine-2-carbonyl)-hexahydro-pyrrolo[3,2-b]pyrrole-1-carbonyl]-butyl}-benzamide;
303. N-{3-Methyl-1-[6-oxo-4-(pyridine-2-carbonyl)-hexahydro-pyrrolo[3,2-b]pyrrole-1-carbonyl]-butyl}-4-thiophen-2-yl-benzamide;
304. N-{3-Methyl-1-[6-oxo-4-(pyridine-2-carbonyl)-hexahydro-pyrrolo[3,2-b]pyrrole-1-carbonyl]-butyl}-4-oxazol-5-yl-benzamide;
305. 5-Phenyl-thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
306. N-{3-Methyl-1-[6-oxo-4-(pyridine-2-carbonyl)-hexahydro-pyrrolo[3,2-b]pyrrole-1-carbonyl]-butyl}-4-trifluoromethoxy-benzamide;
307. 5-Pyridin-2-yl-thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
308. 4-Difluoromethoxy-N-{3-methyl-1-[6-oxo-4-(pyridine-2-carbonyl)-hexahydro-pyrrolo[3,2-b]pyrrole-1-carbonyl]-butyl}-benzamide;
309. N-{3-Methyl-1-[6-oxo-4-(pyridine-2-carbonyl)-hexahydro-pyrrolo[3,2-b]pyrrole-1-carbonyl]-butyl}-4-morpholin-4-yl-benzamide;
310. Naphthalene-2-carboxylic acid {1-[6-oxo-4-(pyridine-2-carbonyl)hexahydro-pyrrolo[3,2-b]pyrrole-1-carbonyl]-butyl}-amide;
311. Quinoline-2-carboxylic acid {1-[6-oxo-4-(pyridine-2-carbonyl)-hexahydro-pyrrolo[3,2-b]pyrrole-1-carbonyl]-butyl}-amide;
312. Benzo[b]thiophene-2-carboxylic acid {1-[6-oxo-4-(pyridine-2-carbonyl)-hexahydro-pyrrolo[3,2-b]pyrrole-1-carbonyl]-butyl}-amide;
313. Benzofuran-2-carboxylic acid {1-[6-oxo-4-(pyridine-2-carbonyl)-hexahydro-pyrrolo[3,2-b]pyrrole-1-carbonyl]-butyl}-amide;
314. Biphenyl-4-carboxylic acid {1-[6-oxo-4-(pyridine-2-carbonyl)-hexahydro-pyrrolo[3,2-b]pyrrole-1-carbonyl]-butyl}-amide;
315. 4-tert-Butyl-N-{1-[6-oxo-4-(pyridine-2-carbonyl)-hexahydro-pyrrolo[3,2-b]pyrrole-1-carbonyl]-butyl}-benzamide;
316. 4-Dimethylamino-N-{1-[6-oxo-4-(pyridine-2-carbonyl)hexahydro-pyrrolo[3,2-b]pyrrole-1-carbonyl]-butyl}-benzamide;
317. 7-Methoxy-benzofuran-2-carboxylic acid {1-[6-oxo-4-(pyridine-2-carbonyl)-hexahydro-pyrrolo[3,2-b]pyrrole-1-carbonyl]-butyl}-amide;
318. {-[6-oxo-4-(pyridine-2-carbonyl)-hexahydro-pyrrolo[3,2-b]pyrrole-1-carbonyl]-butyl}-carbamic acid benzyl ester;
319. 5-Methoxy-benzofuran-2-carboxylic acid {1-[6-oxo-4-(pyridine-2-carbonyl)-hexahydro-pyrrolo[3,2-b]pyrrole-1-carbonyl]-butyl}-amide;
320. Thieno[3,2-b]thiophene-2-carboxylic acid {1-[6-oxo-4-(pyridine-2-carbonyl)-hexahydro-pyrrolo[3,2-b]pyrrole-1-carbonyl]-butyl}-amide;
321. 3-Methyl-benzofuran-2-carboxylic acid {1-[6-oxo-4-(pyridine-2-carbonyl)-hexahydro-pyrrolo[3,2-b]pyrrole-1-carbonyl]-butyl}-amide;
322. Quinoxaline-2-carboxylic acid {1-[6-oxo-4-(pyridine-2-carbonyl)-hexahydro-pyrrolo[3,2-b]pyrrole-1-carbonyl]-butyl}-amide;
323. Benzo[1,3]dioxole-5-carboxylic acid {1-[6-oxo-4-(pyridine-2-carbonyl)-hexahydro-pyrrolo[3,2-b]pyrrole-1-carbonyl]-butyl}-amide;
324. Quinoline-6-carboxylic acid {1-[6-oxo-4-(pyridine-2-carbonyl)-hexahydro-pyrrolo[3,2-b]pyrrole-1-carbonyl]-butyl}-amide;
325. Furan-2-carboxylic acid {1-[6-oxo-4-(pyridine-2-carbonyl)-hexahydro-pyrrolo[3,2-b]pyrrole-1-carbonyl]-butyl}-amide;
326. Thiophene-2-carboxylic acid {1-[6-oxo-4-(pyridine-2-carbonyl)-hexahydro-pyrrolo[3,2-b]pyrrole-1-carbonyl]-butyl}-amide;
327. N-{1-[6-oxo-4-(pyridine-2-carbonyl)-hexahydro-pyrrolo[3,2-b]pyrrole-1-carbonyl]-butyl}-4-trifluoromethyl-benzamide;
328. Furan-3-carboxylic acid {1-[6-oxo-4-(pyridine-2-carbonyl)-hexahydro-pyrrolo[3,2-b]pyrrole-1-carbonyl]-butyl}-amide;
329. Thiophene-3-carboxylic acid {1-[6-oxo-4-(pyridine-2-carbonyl)-hexahydro-pyrrolo[3,2-b]pyrrole-1-carbonyl]-butyl}-amide;
330. N-{1-[6-oxo-4-(pyridine-2-carbonyl)hexahydro-pyrrolo[3,2-b]pyrrole-1-carbonyl]-butyl}-4-phenoxy-benzamide;
331. N-{1-[6-oxo-4-(pyridine-2-carbonyl)-hexahydro-pyrrolo[3,2-b]pyrrole-1-carbonyl]-butyl}-4-(4-methyl-piperazin-1-yl)-benzamide;
332. 4-Methyl-N-{1-[6-oxo-4-(pyridine-2-carbonyl)-hexahydro-pyrrolo[3,2-b]pyrrole-1-carbonyl]-butyl}-benzamide;
333. 4-Methoxy-N-{1-[6-oxo-4-(pyridine-2-carbonyl)hexahydro-pyrrolo[3,2-b]pyrrole-1-carbonyl]-butyl}-benzamide;
334. 4-Isopropyl-N-{1-[6-oxo-1 (pyridine-2-carbonyl)-hexahydro-pyrrolo[3,2-b]pyrrole-1-carbonyl]-butyl}-benzamide;
335. N-{1-[6-oxo-4-(pyridine-2-carbonyl)-hexahydro-pyrrolo[3,2-b]pyrrole-1-carbonyl]-butyl}-4-vinyl-benzamide;
336. 4-Imidazol-1-yl-N-{1-[6-oxo-4-(pyridine-2-carbonyl)-hexahydro-pyrrolo[3,2-b]pyrrole-1-carbonyl]-butyl}-benzamide;
337. N-{1-[6-oxo-4-(pyridine-2-carbonyl)-hexahydro-pyrrolo[3,2-b]pyrrole-1-carbonyl]-butyl}-4-thiophen-2-yl-benzamide;
338. N-{1-[6-oxo-4-(pyridine-2-carbonyl)-hexahydro-pyrrolo[3,2-b]pyrrole-1-carbonyl]-butyl}-4-oxazol-5-yl-benzamide;
339. 5-Phenyl-thiophene-2-carboxylic acid {1-[6-oxo-4-(pyridine-2-carbonyl)-hexahydro-pyrrolo[3,2-b]pyrrole-1-carbonyl]-butyl}-amide;
340. N-{1-[6-oxo-4-(pyridine-2-carbonyl)-hexahydro-pyrrolo[3,2-b]pyrrole-1-carbonyl]-butyl}-4-trifluoromethoxy-benzamide;
341. 5-Pyridin-2-yl-thiophene-2-carboxylic acid {1-[6-oxo-4-(pyridine-2-carbonyl)-hexahydro-pyrrolo[3,2-b]pyrrole-1-carbonyl]-butyl}-amide;
342. 4-Difluoromethoxy-N-{1-[6-oxo-4-(pyridine-2-carbonyl)-hexahydro-pyrrolo[3,2-b]pyrrole-1-carbonyl]-butyl}-benzamide;
343. N-{1-[6-oxo-4-(pyridine-2-carbonyl)-hexahydro-pyrrolo[3,2-b]pyrrole-1-carbonyl]-butyl}-4-morpholin-4-yl-benzamide;
344. Naphthalene-2-carboxylic acid {3-methyl-1-[6-oxo-1-(2-pyridine-3-yl-acetyl)hexahydro-pyrrolo[3,2-b]pyrrole-1-carbonyl]-butyl}-amide;
345. Quinoline-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
346. Benzo[b]thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
347. Benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
348. Biphenyl-4-carboxylic acid {3-methyl-1-[6-oxo-4-(2-pyridin-3-yl-acetyl) hexahydro-pyrrolo[3,2-b]pyrrole-1-carbonyl]-butyl}-amide;
349. 4-tert-Butyl-N-{3-methyl-1-[6-oxo-4-(2-pyridin-3-yl-acetyl)-hexahydro-pyrrolo[3,2-b]pyrrole-1-carbonyl]-butyl}-benzamide;
350. 4-Dimethylamino-N-{3-methyl-1-[6-oxo-4-(2-pyridin-3-yl-acetyl)-hexahydro-pyrrolo[3,2-b]pyrrole-1-carbonyl]-butyl}-benzamide;
351. 7-Methoxy-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
352. {3-Methyl-1-[6-oxo-4-(2-pyridin-3-yl-acetyl)-hexahydro-pyrrolo[3,2-b]pyrrole-1-carbonyl]-butyl}-carbamic acid benzyl ester;
353. 5-Methoxy-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
354. Thieno[3,2-b]thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
355. 3-Methyl-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
356. Quinoxaline-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
357. Benzo[1,3]dioxole-5-carboxylic acid {3-methyl-1-[6-oxo-4-(2-pyridin-3-yl-acetyl)-hexahydro-pyrrolo[3,2-b]pyrrole-1-carbonyl]-butyl}-amide;
358. Quinoline-6-carboxylic acid {3-methyl-1-[6-oxo-4-(2-pyridin-3-yl-acetyl)-hexahydro-pyrrolo[3,2-b]pyrrole-1-carbonyl]-butyl}-amide;
359. Furan-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
360. Thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
361. N-{3-Methyl-1-[6-oxo-4-(2-pyridin-3-yl-acetyl)-hexahydro-pyrrolo[3,2-b]pyrrole-1-carbonyl]-butyl}-4-trifluoromethyl-benzamide;
362. Furan-3-carboxylic acid {3-methyl-1-[6-oxo-4-(2-pyridin-3-yl-acetyl)-hexahydro-pyrrolo[3,2-b]pyrrole-1-carbonyl]-butyl}-amide;
363. Thiophene-3-carboxylic acid {3-methyl-1-[6-oxo-4-(2-pyridin-3-yl-acetyl)-hexahydro-pyrrolo[3,2-b]pyrrole-1-carbonyl]-butyl}-amide;
364. N-{3-Methyl-1-[6-oxo-(2-pyridin-3-yl-acetyl)-hexahydro-pyrrolo[3,2-b]pyrrole-1-carbonyl]-butyl}-4-phenoxy-benzamide;
365. N-{3-Methyl-1-[6-oxo-(2-pyridin-3-yl-acetyl)-hexahydro-pyrrolo[3,2-b]pyrrole-1-carbonyl]-butyl}-4-(4-methyl-piperazin-1-yl)-benzamide;
366. 4-Methyl-N-{3-methyl-1-[6-oxo-4-(2-pyridin-3-yl-acetyl)-hexahydro-pyrrolo[3,2-b]pyrrole-1-carbonyl]-butyl}-benzamide;
367. 4-Methoxy-N-{3-methyl-1-[6-oxo-4-(2-pyridin-3-yl-acetyl)-hexahydro-pyrrolo[3,2-b]pyrrole-1-carbonyl]-butyl}-benzamide;
368. 4-Isopropyl-N-{3-methyl-1-[6-oxo-4-(2-pyridin-3-yl-acetyl)-hexahydro-pyrrolo[3,2-b]pyrrole-1-carbonyl]-butyl}-benzamide;
369. N-{3-Methyl-1-[6-oxo-4-(2-pyridin-3-yl-acetyl)-hexahydro-pyrrolo[3,2-b]pyrrole-1-carbonyl]-butyl}-4-vinyl-benzamide;
370. 4-Imidazol-1-yl-N-{3-methyl-1-[6-oxo-4-(2-pyridin-3-yl-acetyl)-hexahydro-pyrrolo[3,2-b]pyrrole-1-carbonyl]-butyl}-benzamide;
371. N-{3-Methyl-1-[6-oxo-4-(2-pyridin-3-yl-acetyl)-hexahydro-pyrrolo[3,2-b]pyrrole-1-carbonyl]-butyl}-4-thiophen-2-yl-benzamide;
372. N-{3-Methyl-1-[6-oxo-4-(2-pyridin-3-yl-acetyl)-hexahydro-pyrrolo[3,2-b]pyrrole-1-carbonyl]-butyl}-oxazol-5-yl-benzamide;
373. 5-Phenyl-thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
374. N-{3-Methyl-1-[6-oxo-4-(2-pyridin-3-yl-acetyl)-hexahydro-pyrrolo[3,2-b]pyrrole-1-carbonyl]-butyl}-4-trifluoromethoxy-benzamide;
375. 5-Pyridin-2-yl-thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
376. 4-Difluoromethoxy-N-{3-methyl-1-[6-oxo-4-(2-pyridin-3-yl-acetyl)-hexahydro-pyrrolo[3,2-b]pyrrole-1-carbonyl]-butyl}-benzamide;
377. N-{3-Methyl-1-[6-oxo-4-(2-pyridin-3-yl-acetyl)-hexahydro-pyrrolo[3,2-b]pyrrole-1-carbonyl]-butyl}-4-morpholin-4-yl-benzamide;
378. Naphthalene-2-carboxylic acid {1-[6-oxo-4-(2-pyridin-3-yl-acetyl)hexahydro-pyrrolo[3,2-b]pyrrole-1-carbonyl]-butyl}-amide;
379. Quinoline-2-carboxylic acid {1-[6-oxo-4-(2-pyridin-3-yl-acetyl) hexahydro-pyrrolo[3,2-b]pyrrole-1-carbonyl]-butyl}-amide;
380. Benzo[b]thiophene-2-carboxylic acid {1-[6-oxo-4-(2-pyridin-3-yl-acetyl)-hexahydro-pyrrolo[3,2-b]pyrrole-1-carbonyl]-butyl}-amide;
381. Benzofuran-2-carboxylic acid {1-[6-oxo-4-(2-pyridin-3-yl-acetyl) hexahydro-pyrrolo[3,2-b]pyrrole-1-carbonyl]-butyl}-amide;
382. Biphenyl-4-carboxylic acid {1-[6-oxo-4-(2-pyridin-3-yl-acetyl)-hexahydro-pyrrolo[3,2-b]pyrrole-1-carbonyl]-butyl}-amide;
383. 4-tert-Butyl-N-{1-[6-oxo-4-(2-pyridin-3-yl-acetyl)-hexahydro-pyrrolo[3,2-b]pyrrole-1-carbonyl]-butyl}-benzamide;
384. 4-Dimethylamino-N-{1-[6-oxo-4-(2-pyridin-3-yl-acetyl)-hexahydro-pyrrolo[3,2-b]pyrrole-1-carbonyl]-butyl}-benzamide;
385. 7-Methoxy-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
386. {1-[6-oxo-4-(2-pyridin-3-yl-acetyl)-hexahydro-pyrrolo[3,2-b]pyrrole-1-carbonyl]-butyl}-carbamic acid benzyl ester;
387. 5-Methoxy-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
388. Thieno[3,2-b]thiophene-2-carboxylic acid {1-[6-oxo-4-(2-pyridin-3-yl-acetyl)-hexahydro-pyrrolo[3,2-b]pyrrole-1-carbonyl]-butyl}-amide;
389. 3-Methyl-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
390. Quinoxaline-2-carboxylic acid {1-[6-oxo-4-(2-pyridin-3-yl-acetyl)-hexahydro-pyrrolo[3,2-b]pyrrole-1-carbonyl]-butyl}-amide;
391. Benzo[1,3]dioxole-5-carboxylic acid {1-[6-oxo-4-(2-pyridin-3-yl-acetyl)-hexahydro-pyrrolo[3,2-b]pyrrole-1-carbonyl]-butyl}-amide;
392. Quinoline-6-carboxylic acid {1-[6-oxo-4-(2-pyridin-3-yl-acetyl)-hexahydro-pyrrolo[3,2-b]pyrrole-1-carbonyl]-butyl}-amide;
393. Furan-2-carboxylic acid {1-[6-oxo-4-(2-pyridin-3-yl-acetyl)hexahydro-pyrrolo[3,2-b]pyrrole-1-carbonyl]-butyl}-amide;
394. Thiophene-2-carboxylic acid {1-[6-oxo-4-(2-pyridin-3-yl-acetyl)-hexahydro-pyrrolo[3,2-b]pyrrole-1-carbonyl]-butyl}-amide;
395. N-{-[6-oxo-4-(2-pyridin-3-yl-acetyl)-hexahydro-pyrrolo[3,2-b]pyrrole-1-carbonyl]-butyl}-4-trifluoromethyl-benzamide;
396. Furan-3-carboxylic acid {1-[6-oxo-4-(2-pyridin-3-yl-acetyl)-hexahydro-pyrrolo[3,2-b]pyrrole-1-carbonyl]-butyl}-amide;
397. Thiophene-3-carboxylic acid {1-[6-oxo-4-(2-pyridin-3-yl-acetyl)-hexahydro-pyrrolo[3,2-b]pyrrole-1-carbonyl]-butyl}-amide;
398. N-{1-[6-oxo-4-(2-pyridin-3-yl-acetyl)-hexahydro-pyrrolo[3,2-b]pyrrole-1-carbonyl]-butyl}-4-phenoxy-benzamide;
399. N-{1-[6-oxo-4-(2-pyridin-3-yl-acetyl)hexahydro-pyrrolo[3,2-b]pyrrole-1-carbonyl]-butyl}-4-(4-methyl-piperazin-1-yl)-benzamide;
400. 4-Methyl-N-{1-[6-oxo-(2-pyridin-3-yl-acetyl)-hexahydro-pyrrolo[3,2-b]pyrrole-1-carbonyl]-butyl}-benzamide;
401. 4-Methoxy-N-{1-[6-oxo-4-(2-pyridin-3-yl-acetyl)-hexahydro-pyrrolo[3,2-b]pyrrole-1-carbonyl]-butyl}-benzamide;
402. 4-Isopropyl-N-{1-[6-oxo-4-(2-pyridin-3-yl-acetyl)-hexahydro-pyrrolo[3,2-b]pyrrole-1-carbonyl]-butyl}-benzamide;
403. N-{1-[6-oxo-4-(2-pyridin-3-yl-acetyl)-hexahydro-pyrrolo[3,2-b]pyrrole-1-carbonyl]-butyl}-4-vinyl-benzamide;
404. 4-imidazol-1-yl-N-{1-[6-oxo-4-(2-pyridin-3-yl-acetyl)-hexahydro-pyrrolo[3,2-b]pyrrole-1-carbonyl]-butyl}-benzamide;
405. N-{1-[6-oxo-4-(2-pyridin-3-yl-acetyl)-hexahydro-pyrrolo[3,2-b]pyrrole-1-carbonyl]-butyl}-4-thiophen-2-yl-benzamide;
406. N-{1-[6-oxo-4-(2-pyridin-3-yl-acetyl)-hexahydro-pyrrolo[3,2-b]pyrrole-1-carbonyl]-butyl}-4-oxazol-5-yl-benzamide;
407. 5-Phenyl-thiophene-2-carboxylic acid {1-[6-oxo-4-(2-pyridin-3-yl-acetyl)-hexahydro-pyrrolo[3,2-b]pyrrole-1-carbonyl]-butyl}-amide;
408. N-{1-[6-oxo-4-(2-pyridin-3-yl-acetyl)hexahydro-pyrrolo[3,2-b]pyrrole-1-carbonyl]-butyl}-4-trifluoromethoxy-benzamide;
409. 5-Pyridin-2-yl-thiophene-2-carboxylic acid {1-[6-oxo-4-(2-pyridin-3-yl-acetyl)-hexahydro-pyrrolo[3,2-b]pyrrole-1-carbonyl]-butyl}-amide;
410. 4-Difluoromethoxy-N-{1-[6-oxo-4-(2-pyridin-3-yl-acetyl)-hexahydro-pyrrolo[3,2-b]pyrrole-1-carbonyl]-butyl}-benzamide;
411. N-{1-[6-oxo-4-(2-pyridin-3-yl-acetyl)-hexahydro-pyrrolo[3,2-b]pyrrole-1-carbonyl]-butyl}-4-morpholin-4-yl-benzamide;
412. Naphthalene-2-carboxylic acid {3-methyl-1-[6-oxo-4-(pyridin-2-ylmethanesulfonyl)hexahydro-pyrrolo[3,2-b]pyrrole-1-carbonyl]-butyl}-amide;
413. Quinoline-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
414. Benzo[b]thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
415. Benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
416. Biphenyl-4-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
417. 4-tert-Butyl-N-{3-methyl-1-[6-oxo-4-(pyridin-2-ylmethanesulfonyl)-hexahydro-pyrrolo[3,2-b]pyrrole-1-carbonyl]-butyl}-benzamide;
418. 4-Dimethylamino-N-{3-methyl-1-[6-oxo-4-(pyridin-2-ylmethanesulfonyl)-hexahydro-pyrrolo[3,2-b]pyrrole-1-carbonyl]-butyl}-benzamide;
419. 7-Methoxy-benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
420. {3-Methyl-1-[6-oxo-4-(pyridin-2-ylmethanesulfonyl)-hexahydro-pyrrolo[3,2-b]pyrrole-1-carbonyl]-butyl}-carbamic acid benzyl ester;
421. 5-Methoxy-benzofuran-2-carboxylic acid {3-methyl-1-[6-oxo-4-(pyridin-2-ylmethanesulfonyl)hexahydro-pyrrolo[3,2-b]pyrrole-1-carbonyl]-butyl}-amide;
422. Thieno[3,2-b]thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
423. 3-Methyl-benzofuran-2-carboxylic acid {3-methyl-1-[6-oxo-4-(pyridin-2-ylmethanesulfonyl)hexahydro-pyrrolo[3,2-b]pyrrole-1-carbonyl]-butyl}-amide;
424. Quinoxaline-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
425. Benzo[1,3]dioxole-5-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
426. Quinoline-6-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
427. Furan-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
428. Thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
429. N-{3-Methyl-1-[6-oxo-4-(pyridin-2-ylmethanesulfonyl)-hexahydro-pyrrolo[3,2-b]pyrrole-1-carbonyl]-butyl}-4-trifluoromethyl-benzamide,
430. Furan-3-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
431. Thiophene-3-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
432. N-{3-Methyl-1-[6-oxo-4-(pyridin-2-ylmethanesulfonyl)-hexahydro-pyrrolo[3,2-b]pyrrole-1-carbonyl]-butyl}-4-phenoxy-benzamide;
433. N-{3-Methyl-1-[6-oxo-4-(pyridin-2-ylmethanesulfonyl)-hexahydro-pyrrolo[3,2-b]pyrrole-1-carbonyl]-butyl}-4-(4-methyl-piperazin-1-yl)-benzamide;
434. 4-Methyl-N-{3-methyl-1-[6-oxo-4-(pyridin-2-ylmethanesulfonyl)-hexahydro-pyrrolo[3,2-b]pyrrole-1-carbonyl]-butyl}-benzamide;
435. 4-Methoxy-N-{3-methyl-1-[6-oxo-4-(pyridin-2-ylmethanesulfonyl)-hexahydro-pyrrolo[3,2-b]pyrrole-1-carbonyl]-butyl}-benzamide;
436. 4-Isopropyl-N-{3-methyl-1-[6-oxo-4-(pyridin-2-ylmethanesulfonyl)-hexahydro-pyrrolo[3,2-b]pyrrole-1-carbonyl]-butyl}-benzamide;
437. N-{3-Methyl-1-[6-oxo-4-(pyridin-2-ylmethanesulfonyl)-hexahydro-pyrrolo[3,2-b]pyrrole-1-carbonyl]-butyl}-4-vinyl-benzamide;
438. 4-Imidazol-1-yl-N-{3-methyl-1-[6-oxo-4-(pyridin-2-ylmethanesulfonyl) hexahydro-pyrrolo[3,2-b]pyrrole-1-carbonyl]-butyl}-benzamide;
439. N-{3-Methyl-1-[6-oxo-4-(pyridin-2-ylmethanesulfonyl)-hexahydro-pyrrolo[3,2-b]pyrrole-1-carbonyl]-butyl}-4-thiophen-2-yl-benzamide;
440. N-{3-Methyl-1-[6-oxo-4-(pyridin-2-ylmethanesulfonyl)-hexahydro-pyrrolo[3,2-b]pyrrole-1-carbonyl]-butyl}-4-oxazol-5-yl-benzamide;
441. 5-Phenyl-thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
442. N-{3-Methyl-1-[6-oxo-4-(pyridin-2-ylmethanesulfonyl)-hexahydro-pyrrolo[3,2-b]pyrrole-1-carbonyl]-butyl}-4-trifluoromethoxy-benzamide;
443. 5-Pyridin-2-yl-thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
444. 4-Difluoromethoxy-N-{3-methyl-1-[6-oxo-4-(pyridin-2-ylmethanesulfonyl)-hexahydro-pyrrolo[3,2-b]pyrrole-1-carbonyl]-butyl}-benzamide;
445. N-{3-Methyl-1-[6-oxo-4-(pyridin-2-ylmethanesulfonyl)-hexahydro-pyrrolo[3,2-b]pyrrole-1-carbonyl]-butyl}-4-morpholin-4-yl-benzamide;
446. Naphthalene-2-carboxylic acid {1-[6-oxo-4-(pyridin-2-ylmethanesulfonyl)hexahydro-pyrrolo[3,2-b]pyrrole-1-carbonyl]-butyl}-amide;
447. Quinoline-2-carboxylic acid {1-[6-oxo-4-(pyridin-2-ylmethanesulfonyl)-hexahydro-pyrrolo[3,2-b]pyrrole-1-carbonyl]-butyl}-amide;
448. Benzo[b]thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
449. Benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
450. Biphenyl-4-carboxylic acid {1-[6-oxo-4-(pyridin-2-ylmethanesulfonyl)-hexahydro-pyrrolo[3,2-b]pyrrole-1-carbonyl]-butyl}-amide;
451. 4-tert-Butyl-N-{1-[6-oxo-4-(pyridin-2-ylmethanesulfonyl)-hexahydro-pyrrolo[3,2-b]pyrrole-1-carbonyl]-butyl}-benzamide;
452. 4-Dimethylamino-N-{1-[6-oxo-4-(pyridin-2-ylmethanesulfonyl)-hexahydro-pyrrolo[3,2-b]pyrrole-1-carbonyl]-butyl}-benzamide;
453. 7-Methoxy-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
454. {1-[6-oxo-4-(pyridin-2-ylmethanesulfonyl)-hexahydro-pyrrolo[3,2-b]pyrrole-1-carbonyl]-butyl}-carbamic acid benzyl ester;
455. 5-Methoxy-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
456. Thieno[3,2-b]thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
457. 3-Methyl-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
458. Quinoxaline-2-carboxylic acid {1-[6-oxo-4-(pyridin-2-ylmethanesulfonyl)-hexahydro-pyrrolo[3,2-b]pyrrole-1-carbonyl]-butyl}-amide;
459. Benzo[1,3]dioxole-5-carboxylic acid {1-[6-oxo-4-(pyridin-2-ylmethanesulfonyl)-hexahydro-pyrrolo[3,2-b]pyrrole-1-carbonyl]-butyl}-amide;
460. Quinoline-6-carboxylic acid {1-[6-oxo-4-(pyridin-2-ylmethanesulfonyl) hexahydro-pyrrolo[3,2-b]pyrrole-1-carbonyl]-butyl}-amide;
461. Furan-2-carboxylic acid {1-[6-oxo-4-(pyridin-2-ylmethanesulfonyl) hexahydro-pyrrolo[3,2-b]pyrrole-1-carbonyl]-butyl}-amide;
462. Thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
463. N-{1-[6-oxo-4-(pyridin-2-ylmethanesulfonyl)-hexahydro-pyrrolo[3,2-b]pyrrole-1-carbonyl]-butyl}-4-trifluoromethyl-benzamide;
464. Furan-3-carboxylic acid {1-[6-oxo-4-(pyridin-2-ylmethanesulfonyl)-hexahydro-pyrrolo[3,2-b]pyrrole-1-carbonyl]-butyl}-amide;
465. Thiophene-3-carboxylic acid {1-[6-oxo-4-(pyridin-2-ylmethanesulfonyl) hexahydro-pyrrolo[3,2-b]pyrrole-1-carbonyl]-butyl}-amide;
466. N-{1-[6-oxo-4-(pyridin-2-ylmethanesulfonyl)-hexahydro-pyrrolo[3,2-b]pyrrole-1-carbonyl]-butyl}-4-phenoxy-benzamide;
467. N-{1-[6-oxo-4-(pyridin-2-ylmethanesulfonyl)-hexahydro-pyrrolo[3,2-b]pyrrole-1-carbonyl]-butyl}-4-(4-methyl-piperazin-1-yl)-benzamide;
468. 4-Methyl-N-{1-[6-oxo-4-(pyridin-2-ylmethanesulfonyl)-hexahydro-pyrrolo[3,2-b]pyrrole-1-carbonyl]-butyl}-benzamide;
469. 4-Methoxy-N-{1-[6-oxo-4-(pyridin-2-ylmethanesulfonyl)-hexahydro-pyrrolo[3,2-b]pyrrole-1-carbonyl]-butyl}-benzamide;
470. 4-Isopropyl-N-{1-[6-oxo-4-(pyridin-2-ylmethanesulfonyl)-hexahydro-pyrrolo[3,2-b]pyrrole-1-carbonyl]-butyl}-benzamide;
471. N-{1-[6-oxo-4-(pyridin-2-ylmethanesulfonyl)-hexahydro-pyrrolo[3,2-b]pyrrole-1-carbonyl]-butyl}-4-vinyl-benzamide;
472. 4-Imidazol-1-yl-N-{1-[6-oxo-4-(pyridin-2-ylmethanesulfonyl)-hexahydro-pyrrolo[3,2-b]pyrrole-1-carbonyl]-butyl}-benzamide;
473. N-{1-[6-oxo-4-(pyridin-2-ylmethanesulfonyl)-hexahydro-pyrrolo[3,2-b]pyrrole-1-carbonyl]-butyl}-4-thiophen-2-yl-benzamide;
474. N-{1-[6-oxo-4-(pyridin-2-ylmethanesulfonyl)-hexahydro-pyrrolo[3,2-b]pyrrole-1-carbonyl]-butyl}-4-oxazol-5-yl-benzamide;
475. 5-Phenyl-thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
476. N-{1-[6-oxo-4-(pyridin-2-ylmethanesulfonyl)-hexahydro-pyrrolo[3,2-b]pyrrole-1-carbonyl]-butyl}-4-trifluoromethoxy-benzamide;
477. 5-Pyridin-2-yl-thiophene-2-carboxylic acid {1-[6-oxo-4-(pyridin-2-ylmethanesulfonyl)-hexahydro-pyrrolo[3,2-b]pyrrol-carbonyl]-butyl}-amide;
478. 4-Difluoromethoxy-N-{1-[6-oxo-4-(pyridin-2-ylmethanesulfonyl)-hexahydro-pyrrolo[3,2-b]pyrrole-1-carbonyl]-butyl}-benzamide;
479. N-{1-[6-oxo-4-(pyridin-2-ylmethanesulfonyl)hexahydro-pyrrolo[3,2-b]pyrrole-1-carbonyl]-butyl}-4-morpholin-4-yl-benzamide;
480. Naphthalene-2-carboxylic acid {3-methyl-1-[6-oxo-4-(pyridin-3-ylmethanesulfonyl)hexahydro-pyrrolo[3,2-b]pyrrole-1-carbonyl]-butyl}-amide;
481. Quinoline-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
482. Benzo[b]thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
483. Benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
484. Biphenyl-4-carboxylic acid {3-methyl-1-[6-oxo-4-pyridin-3-ylmethanesulfonyl)-hexahydro-pyrrolo[3,2-b]pyrrole-1-carbonyl]-butyl}-amide;
485. 4-tert-Butyl-N-{3-methyl-1-[6-oxo-4-(pyridin-3-ylmethanesulfonyl)-hexahydro-pyrrolo[3,2-b]pyrrole-1-carbonyl]-butyl}-benzamide;
486. 4-Dimethylamino-N-{3-methyl-1-[6-oxo-4-(pyridin-3-ylmethanesulfonyl)-hexahydro-pyrrolo[3,2-b]pyrrole-1-carbonyl]-butyl}-benzamide;
487. 7-Methoxy-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
488. {3-Methyl-1-[6-oxo-4-(pyridin-3-ylmethanesulfonyl)-hexahydro-pyrrolo[3,2-b]pyrrole-1-carbonyl]-butyl}-carbamic acid benzyl ester;
489. 5-Methoxy-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
490. Thieno[3,2-b]thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
491. 3-Methyl-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
492. Quinoxaline-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
493. Benzo[1,3]dioxole-5-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
494. Quinoline-6-carboxylic acid {3-methyl-1-[6-oxo-4-(pyridin-3-ylmethanesulfonyl)hexahydro-pyrrolo[3,2-b]pyrrole-1-carbonyl]-butyl}-amide;
495. Furan-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
496. Thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
497. N-{3-Methyl-1-[6-oxo-4-(pyridin-3-ylmethanesulfonyl)-hexahydro-pyrrolo[3,2-b]pyrrole-1-carbonyl]-butyl}-4-trifluoromethyl-benzamide;
498. Furan-3-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
499. Thiophene-3-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
500. N-{3-Methyl-1-[6-oxo-4-(pyridin-3-ylmethanesulfonyl)hexahydro-pyrrolo[3,2-b]pyrrole-1-carbonyl]-butyl}-4-phenoxy-benzamide;
501. N-{3-Methyl-1-[6-oxo-4-(pyridin-3-ylmethanesulfonyl)hexahydro-pyrrolo[3,2-b]pyrrole-1-carbonyl]-butyl}-4-methyl-piperazin-1-yl)-benzamide;
502. 4-Methyl-N-{3-methyl-1-[6-oxo-4-(pyridin-3-ylmethanesulfonyl) hexahydro-pyrrolo[3,2-b]pyrrole-1-carbonyl]-butyl}-benzamide;
503. 4-Methoxy-N-{3-methyl-1-[6-oxo-4-(pyridin-3-ylmethanesulfonyl)-hexahydro-pyrrolo[3,2-b]pyrrole-1-carbonyl]-butyl}-benzamide;
504. 4-Isopropyl-N-{3-methyl-1-[6-oxo-4-(pyridin-3-ylmethanesulfonyl)-hexahydro-pyrrolo[3,2-b]pyrrole-1-carbonyl]-butyl}-benzamide;
505. N-{3-Methyl-1-[6-oxo-4-(pyridin-3-ylmethanesulfonyl)-hexahydro-pyrrolo[3,2-b]pyrrole-1-carbonyl]-butyl}-4-vinyl-benzamide;
506. 4-Imidazol-1-yl-N-{3-methyl-1-[6-oxo-4-(pyridin-3-ylmethanesulfonyl)-hexahydro-pyrrolo[3,2-b]pyrrole-1-carbonyl]-butyl}-benzamide;
507. N-{3-Methyl-1-[6-oxo-4-(pyridin-3-ylmethanesulfonyl)-hexahydro-pyrrolo[3,2-b]pyrrole-1-carbonyl]-butyl}-4-thiophen-2-yl-benzamide;
508. N-{3-Methyl-1-[6-oxo-4-(pyridin-3-ylmethanesulfonyl)-hexahydro-pyrrolo[3,2-b]pyrrole-1-carbonyl]-butyl}-4-oxazol-5-yl-benzamide;
509. 5-Phenyl-thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
510. N-{3-Methyl-1-[6-oxo-4-(pyridin-3-ylmethanesulfonyl)-hexahydro-pyrrolo[3,2-b]pyrrole-1-carbonyl]-butyl}-4-trifluoromethoxy-benzamide;
511. 5-Pyridin-2-yl-thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
512. 4-Difluoromethoxy-N-{3-methyl-1-[6-oxo-4-(pyridin-3-ylmethanesulfonyl)-hexahydro-pyrrolo[3,2-b]pyrrole-1-carbonyl]-butyl}-benzamide;
513. N-{3-Methyl-1-[6-oxo-4-(pyridin-3-ylmethanesulfonyl)-hexahydro-pyrrolo[3,2-b]pyrrole-1-carbonyl]-butyl}-4-morpholin-4-yl-benzamide;
514. Naphthalene-2-carboxylic acid {1-[6-oxo-4-(pyridin-3-ylmethanesulfonyl)hexahydro-pyrrolo[3,2-b]pyrrole-1-carbonyl]-butyl}-amide;
515. Quinoline-2-carboxylic acid {1-[6-oxo-4-(pyridin-3-ylmethanesulfonyl)-hexahydro-pyrrolo[3,2-b]pyrrole-1-carbonyl]-butyl}-amide;
516. Benzo[b]thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
517. Benzofuran-2-carboxylic acid {1-[6-oxo-1-(pyridin-3-ylmethanesulfonyl)-hexahydro-pyrrolo[3,2-b]pyrrole-1-carbonyl]-butyl}-amide;
518. Biphenyl-4-carboxylic acid {1-[6-oxo-4-(pyridin-3-ylmethanesulfonyl)-hexahydro-pyrrolo[3,2-b]pyrrole-1-carbonyl]-butyl}-amide;
519. 4-tert-Butyl-N-{1-[6-oxo-4-(pyridin-3-ylmethanesulfonyl)-hexahydro-pyrrolo[3,2-b]pyrrole-1-carbonyl]-butyl}-benzamide;
520. 4-Dimethylamino-N-{1-[6-oxo-4-(pyridin-3-ylmethanesulfonyl)-hexahydro-pyrrolo[3,2-b]pyrrole-1-carbonyl]-butyl}-benzamide;
521. 7-Methoxy-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
522. {1-[6-oxo-4-(pyridin-3-ylmethanesulfonyl)-hexahydro-pyrrolo[3,2-b]pyrrole-1-carbonyl]-butyl}-carbamic acid benzyl ester;
523. 5-Methoxy-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
524. Thieno[3,2-b]thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
525. 3-Methyl-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
526. Quinoxaline-2-carboxylic acid {1-[6-oxo-4-(pyridin-3-ylmethanesulfonyl)-hexahydro-pyrrolo[3,2-b]pyrrole-1-carbonyl]-butyl}-amide;
527. Benzo[1,3]dioxole-5-carboxylic acid {1-[6-oxo-4-(pyridin-3-ylmethanesulfonyl)-hexahydro-pyrrolo[3,2-b]pyrrole-1-carbonyl]-butyl}-amide;
528. Quinoline-6-carboxylic acid {1-[6-oxo-4-pyridin-3-ylmethanesulfonyl)-hexahydro-pyrrolo[3,2-b]pyrrole-1-carbonyl]-butyl}-amide;
529. Furan-2-carboxylic acid {1-[6-oxo-4-(pyridin-3-ylmethanesulfonyl)-hexahydro-pyrrolo[3,2-b]pyrrole-1-carbonyl]-butyl}-amide;
530. Thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
531. N-{1-[6-oxo-4-(pyridin-3-ylmethanesulfonyl)-hexahydro-pyrrolo[3,2-b]pyrrole-1-carbonyl]-butyl}-4-trifluoromethyl-benzamide;
532. Furan-3-carboxylic acid {1-[6-oxo-(pyridin-3-ylmethanesulfonyl)-hexahydro-pyrrolo[3,2-b]pyrrole-1-carbonyl]-butyl}-amide;
533. Thiophene-3-carboxylic acid {1-[6-oxo-4-(pyridin-3-ylmethanesulfonyl)-hexahydro-pyrrolo[3,2-b]pyrrole-1-carbonyl]-butyl}-amide;
534. N-{1-[6-oxo-4-(pyridin-3-ylmethanesulfonyl)-hexahydro-pyrrolo[3,2-b]pyrrole-1-carbonyl]-butyl}-4-phenoxy-benzamide;
535. N-{1-[6-oxo-4-(pyridin-3-ylmethanesulfonyl)hexahydro-pyrrolo[3,2-b]pyrrole-1-carbonyl]-butyl}-4-(4-methyl-piperazin-1-yl)-benzamide;
536. 4-Methyl-N-{1-[6-oxo-4-(pyridin-3-ylmethanesulfonyl)-hexahydro-pyrrolo[3,2-b]pyrrole-1-carbonyl]-butyl}-benzamide;
537. 4-Methoxy-N-{1-[6-oxo-4-(pyridin-3-ylmethanesulfonyl)-hexahydro-pyrrolo[3,2-b]pyrrole-1-carbonyl]-butyl}-benzamide;
538. 4-Isopropyl-N-{1-[6-oxo-4-(pyridin-3-ylmethanesulfonyl)-hexahydro-pyrrolo[3,2-b]pyrrole-1-carbonyl]-butyl}-benzamide;
539. N-{1-[6-oxo-4-(pyridin-3-ylmethanesulfonyl)-hexahydro-pyrrolo[3,2-b]pyrrole-1-carbonyl]-butyl}-4-vinyl-benzamide;
540. 4-Imidazol-1-yl-N-{1-[6-oxo-4-(pyridin-3-ylmethanesulfonyl)-hexahydro-pyrrolo[3,2-b]pyrrole-1-carbonyl]-butyl}-benzamide;
541. N-{1-[6-oxo-4-(pyridin-3-ylmethanesulfonyl)-hexahydro-pyrrolo[3,2-b]pyrrole-1-carbonyl]-butyl}-4-thiophen-2-yl-benzamide;
542. N-{1-[6-oxo-4-(pyridin-3-ylmethanesulfonyl)-hexahydro-pyrrolo[3,2-b]pyrrole-1-carbonyl]-butyl}-4-oxazol-5-yl-benzamide;
543. 5-Phenyl-thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
544. N-{1-[6-oxo-4-(pyridin-3-ylmethanesulfonyl)hexahydro-pyrrolo[3,2-b]pyrrole-1-carbonyl]-butyl}-4-trifluoromethoxy-benzamide;
545. 5-Pyridin-2-yl-thiophene-2 caboxylic acid {1-[6-oxo-4-(pyridin-3-ylmethanesulfonyl)hexahydro-pyrrolo[3,2-b]pyrrole-1-carbonyl]-butyl}-amide;
546. 4-Difluoromethoxy-N-{1-[6-oxo-4-(pyridin-3-ylmethanesulfonyl)-hexahydro-pyrrolo[3,2-b]pyrrole-1-carbonyl]-butyl}-benzamide;
547. N-{1-[6-oxo-4-(pyridin-3-ylmethanesulfonyl)-hexahydro-pyrrolo[3,2-b]pyrrole-1-carbonyl]-butyl}-4-morpholin-4-yl-benzamide;
548. Naphthalene-2-carboxylic acid {3-methyl-1-[6-oxo-4-(1-oxy-pyridin-2-ylmethanesulfonyl)hexahydro-pyrrolo[3,2-b]pyrrole-1-carbonyl]-butyl}-amide;
549. Quinoline-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
550. Benzo[b]thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
551. Benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
552. Biphenyl-4-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
553. 4-tert-Butyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
554. 4-Dimethylamino-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
555. 7-Methoxy-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
556. {3-Methyl-1-[6-oxo-4-(1-oxy-pyridin-2-ylmethanesulfonyl)-hexahydro-pyrrolo[3,2-b]pyrrole-1-carbonyl]-butyl}-carbamic acid benzyl ester,
557. 5-Methoxy-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
558. Thieno[3,2-b]thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
559. 3-Methyl-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
560. Quinoxaline-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
561. Benzo[1,3]dioxole-5-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
562. Quinoline-6-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
563. Furan-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
564. Thiophene-2-carboxylic acid {3-methyl-1-[6-oxo-4-(1-oxy-pyridin-2-ylmethanesulfonyl)hexahydro-pyrrolo[3,2-b]pyrrole-1-carbonyl]-butyl}-amide;
565. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-trifluoromethyl-benzamide;
566. Furan-3-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
567. Thiophene-3-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
568. N-{3-Methyl-1-[6-oxo-4-(1-oxy-pyridin-2-ylmethanesulfonyl)-hexahydro-pyrrolo[3,2-b]pyrrole-1-carbonyl]-butyl}phenoxy-benzamide;
569. N-{3-Methyl-1-[6-oxo-4-(1-oxy-pyridin-2-ylmethanesulfonyl)hexahydro-pyrrolo[3,2-b]pyrrole-1-carbonyl]-butyl}-4-(4-methyl-piperazin-1-yl)-benzamide;
570. 4-Methyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
571. 4-Methoxy-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
572. 4-Isopropyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
573. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-vinyl-benzamide;
574. 4-Imidazol-1-yl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
575. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-thiophen-2-yl-benzamide;
576. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-oxazol-5-yl-benzamide;
577. 5-Phenyl-thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
578. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-trifluoromethoxy-benzamide;
579. 5-Pyridin-2-yl-thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
580. 4-Difluoromethoxy-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
581. N-{3-Methyl-1-[6-oxo-4-(1-oxy-pyridin-2-ylmethanesulfonyl)-hexahydro-pyrrolo[3,2-b]pyrrole-1-carbonyl]-butyl}-4-morpholin-4-yl-benzamide;
582. Naphthalene-2-carboxylic acid {1-[6-oxo-4-(1-oxy-pyridin-2-ylmethanesulfonyl)hexahydro-pyrrolo[3,2-b]pyrrole-1-carbonyl]-butyl}-amide;
583. Quinoline-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
584. Benzo[b]thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
585. Benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
586. Biphenyl-4-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
587. 4-tert-Butyl-N-{1-[6-oxo-4-(1-oxy-pyridin-2-ylmethanesulfonyl)-hexahydro-pyrrolo[3,2-b]pyrrole-1-carbonyl]-butyl}-benzamide;
588. 4-Dimethylamino-N-{1-[6-oxo-4-(1-oxy-pyridin-2-ylmethanesulfonyl)-hexahydro-pyrrolo[3,2-b]pyrrole-1-carbonyl]-butyl}-benzamide;
589. 7-Methoxy-benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
590. {1-[6-oxo-4-(1-oxy-pyridin-2-ylmethanesulfonyl)-hexahydro-pyrrolo[3,2-b]pyrrole-1-carbonyl]-butyl}-carbamic acid benzyl ester;
591. 5-Methoxy-benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
592. Thieno[3,2-b]thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
593. 3-Methyl-benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
594. Quinoxaline-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
595. Benzo[1,3]dioxole-5-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
596. Quinoline-6-carboxylic acid {1-[6-oxo-4-(1-oxy-pyridin-2-ylmethanesulfonyl)hexahydro-pyrrolo[3,2-b]pyrrole-1-carbonyl]-butyl}-amide;
597. Furan-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
598. Thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
599. N-{1-[6-oxo-4-(1-oxy-pyridin-2-ylmethanesulfonyl)-hexahydro-pyrrolo[3,2-b]pyrrole-1-carbonyl]-butyl}-4-trifluoromethyl-benzamide;
600. Furan-3-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
601. Thiophene-3-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
602. N-{1-[6-oxo-4-(1-oxy-pyridin-2-ylmethanesulfonyl)-hexahydro-pyrrolo[3,2-b]pyrrole-1-carbonyl]-butyl}-4-phenoxy-benzamide;
603. N-{1-[6-oxo-4-(1-oxy-pyridin-2-ylmethanesulfonyl)-hexahydro-pyrrolo[3,2-b]pyrrole-1-carbonyl]-butyl}-4-(4-methyl-piperazin-1-yl)-benzamide;
604. 4-Methyl-N-{1-[6-oxo-4-(1-oxy-pyridin-2-ylmethanesulfonyl)-hexahydro-pyrrolo[3,2-b]pyrrole-1-carbonyl]-butyl}-benzamide;
605. 4-Methoxy-N-{1-[6-oxo-4-(1-oxy-pyridin-2-ylmethanesulfonyl)-hexahydro-pyrrolo[3,2-b]pyrrole-1-carbonyl]-butyl}-benzamide;
606. 4-Isopropyl-N-{1-[6-oxo-4-(1-oxy-pyridin-2-ylmethanesulfonyl)-hexahydro-pyrrolo[3,2-b]pyrrole-1-carbonyl]-butyl}-benzamide;
607. N-{1-[6-oxo-4-(1-oxy-pyridin-2-ylmethanesulfonyl)-hexahydro-pyrrolo[3,2-b]pyrrole-1-carbonyl]-butyl}-4-vinyl-benzamide;
608. 4-Imidazol-1-yl-N-{1-[6-oxo-4-(1-oxy-pyridin-2-ylmethanesulfonyl)-hexahydro-pyrrolo[3,2-b]pyrrole-1-carbonyl]-butyl}-benzamide;
609. N-{1-[6-oxo-4-(1-oxy-pyridin-2-ylmethanesulfonyl)-hexahydro-pyrrolo[3,2-b]pyrrole-1-carbonyl]-butyl}-4-thiophen-2-yl-benzamide;
610. N-{1-[6-oxo-4-(1-oxy-pyridin-2-ylmethanesulfonyl)-hexahydro-pyrrolo[3,2-b]pyrrole-1-carbonyl]-butyl}-4-oxazol-5-yl-benzamide;
611. 5-Phenyl-thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
612. N-{1-[6-oxo-4-(1-oxy-pyridin-2-ylmethanesulfonyl)-hexahydro-pyrrolo[3,2-b]pyrrole-1-carbonyl]-butyl}-4-trifluoromethoxy-benzamide;
613. 5-Pyridin-2-yl-thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
614. 4-Difluoromethoxy-N-{1-[6-oxo-4-(1-oxy-pyridin-2-ylmethanesulfonyl)-hexahydro-pyrrolo[3,2-b]pyrrole-1-carbonyl]-butyl}-benzamide;
615. N-{1-[6-oxo-4-(1-oxy-pyridin-2-ylmethanesulfonyl)-hexahydro-pyrrolo[3,2-b]pyrrole-1-carbonyl]-butyl}-4-morpholin-4-yl-benzamide;
616. Naphthalene-2-carboxylic acid {3-methyl-1-[6-oxo-4-(1-oxy-pyridin-3-ylmethanesulfonyl)hexahydro-pyrrolo[3,2-b]pyrrole-1-carbonyl]-butyl}-amide;
617. Quinoline-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
618. Benzo[b]thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
619. Benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
620. Biphenyl-4-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
621. 4-tert-Butyl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
622. 4-Dimethylamino-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
623. 7-Methoxy-benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
624. {3-Methyl-1-[6-oxo-4-(1-oxy-pyridin-3-ylmethanesulfonyl)-hexahydro-pyrrolo[3,2-b]pyrrole-1-carbonyl]-butyl}-carbamic acid benzyl ester;
625. 5-Methoxy-benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
626. Thieno[3,2-b]thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
627. 3-Methyl-benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
628. Quinoxaline-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
629. Benzo[1,3]dioxole-5-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
630. Quinoline-6-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
631. Furan-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
632. Thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
633. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-trifluoromethyl-benzamide;
634. Furan-3-caboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
635. Thiophene-3-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
636. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-phenoxy-benzamide;
637. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-(4-methyl-piperazin-1-yl) benzamide;
638. 4-Methyl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
639. 4-Methoxy-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
640. 4-Isopropyl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
641. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-vinyl-benzamide;
642. 4-Imidazol-1-yl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
643. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-thiophen-2-yl-benzamide;
644. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-oxazol-5-yl-benzamide;
645. 5-Phenyl-thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
646. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-trifluoromethoxy-benzamide;
647. 5-Pyridin-2-yl-thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
648. 4-Difluoromethoxy-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
649. N-{3-Methyl-1-[6-oxo-4-(1-oxy-pyridin-3-ylmethanesulfonyl)-hexahydro-pyrrolo[3,2-b]pyrrole-1-carbonyl]-butyl}-4-morpholin-4-yl-benzamide;
650. Naphthalene-2-carboxylic acid {1-[6-oxo-4-(1-oxy-pyridin-3-ylmethanesulfonyl)hexahydro-pyrrolo[3,2-b]pyrrole-1-carbonyl]-butyl}-amide;
651. Quinoline-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
652. Benzo[b]thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
653. Benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
654. Biphenyl-4-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
655. 4-tert-Butyl-N-{1-[6-oxo-4-(1-oxy-pyridin-3-ylmethanesulfonyl)-hexahydro-pyrrolo[3,2-b]pyrrole-1-carbonyl]-butyl}-benzamide;
656. 4-Dimethylamino-N-{1-[6-oxo-4-(1-oxy-pyridin-3-ylmethanesulfonyl)-hexahydro-pyrrolo[3,2-b]pyrrole-1-carbonyl]-butyl}-benzamide;
657. 7-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
658. {1-[6-oxo-4-(1-oxy-pyridin-3-ylmethanesulfonyl)-hexahydro-pyrrolo[3,2-b]pyrrole-1-carbonyl]-butyl}-carbamic acid benzyl ester;
659. 5-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
660. Thieno[3,2-b]thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
661. 3-Methyl-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
662. Quinoxaline-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
663. Benzo[1,3]dioxole-5-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
664. Quinoline-6-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
665. Furan-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
666. Thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
667. N-{1-[6-oxo-4-(1-oxy-pyridin-3-ylmethanesulfonyl)-hexahydro-pyrrolo[3,2-b]pyrrole-1-carbonyl]-butyl}-4-trifluoromethyl-benzamide;
668. Furan-3-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
669. Thiophene-3-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
670. N-{1-[6-oxo-4-(1-oxy-pyridin-3-ylmethanesulfonyl)-hexahydro-pyrrolo[3,2-b]pyrrole-1-carbonyl]-butyl}-4-phenoxy-benzamide;
671. N-{1-[6-oxo-4-(1-oxy-pyridin-3-ylmethanesulfonyl)-hexahydro-pyrrolo[3,2-b]pyrrole-1-carbonyl]-butyl}-4-(4-methyl-piperazin-1-yl)-benzamide;
672. 4-Methyl-N-{1-[6-oxo-4-(1-oxy-pyridin-3-ylmethanesulfonyl)hexahydro-pyrrolo[3,2-b]pyrrole-1-carbonyl]-butyl}-benzamide;
673. 4-Methoxy-N-{1-[6-oxo-4-(1-oxy-pyridin-3-ylmethanesulfonyl)-hexahydro-pyrrolo[3,2-b]pyrrole-1-carbonyl]-butyl}-benzamide;
674. 4-Isopropyl-N-{1-[6-oxo-4-(1-oxy-pyridin-3-ylmethanesulfonyl)-hexahydro-pyrrolo[3,2-b]pyrrole-1-carbonyl]-butyl}-benzamide;
675. N-{1-[6-oxo-4-(1-oxy-pyridin-3-ylmethanesulfonyl)-hexahydro-pyrrolo[3,2-b]pyrrole-1-carbonyl]-butyl}-4-vinyl-benzamide;
676. 4-Imidazol-1-yl-N-{1-[6-oxo-4-(1-oxy-pyridin-3-ylmethanesulfonyl)-hexahydro-pyrrolo[3,2-b]pyrrole-1-carbonyl]-butyl}-benzamide;
677. N-{1-[6-oxo-4-(1-oxy-pyridin-3-ylmethanesulfonyl)-hexahydro-pyrrolo[3,2-b]pyrrole-1-carbonyl]-butyl}-4-thiophen-2-yl-benzamide;
678. N-{1-[6-oxo-4-(1-oxy-pyridin-3-ylmethanesulfonyl)-hexahydro-pyrrolo[3,2-b]pyrrole-1-carbonyl]-butyl}-4-oxazol-5-yl-benzamide;
679. 5-Phenyl-thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
680. N-{1-[6-oxo-4-(1-oxy-pyridin-3-ylmethanesulfonyl)-hexahydro-pyrrolo[3,2-b]pyrrole-1-carbonyl]-butyl}-4-trifluoromethoxy-benzamide;
681. 5-Pyridin-2-yl-thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
682. 4-Difluoromethoxy-N-{1-[6-oxo-4-(1-oxy-pyridin-3-ylmethanesulfonyl)-hexahydro-pyrrolo[3,2-b]pyrrole-1-carbonyl]-butyl}-benzamide;
683. N-{1-[6-oxo-4-(1-oxy-pyridin-3-ylmethanesulfonyl)-hexahydro-pyrrolo[3,2-b]pyrrole-1-carbonyl]-butyl}-4-morpholin-4-yl-benzamide;
684. 4-tert-Butyl-N-{1-[6-oxo-4-(pyridine-2-sulfonyl)-hexahydro-pyrrolo[3,2-b]pyrrole-1-carbonyl]-cyclohexyl}-benzamide;
685. 5-Methoxy-benzofuran-2-carboxylic acid {1-[6-oxo-4-(pyridine-2-sulfonyl)-hexahydro-pyrrolo[3,2-b]pyrrole-1-carbonyl]-cyclohexyl}-amide;
686. Thieno[3,2-b]thiophene-2-carboxylic acid {1-[6-oxo-4-(pyridine-2-sulfonyl)-hexahydro-pyrrolo[3,2-b]pyrrole-1-carbonyl]-cyclohexyl}-amide;
687. 3-Methyl-benzofuran-2-carboxylic acid {1-[6-oxo-4-(pyridine-2-sulfonyl) hexahydro-pyrrolo[3,2-b]pyrrole-1-carbonyl]-cyclohexyl}-amide;
688. N-{1-[6-oxo-4-(pyridine-2-sulfonyl)-hexahydro-pyrrolo[3,2-b]pyrrole-1-carbonyl]-cyclohexyl}-4-phenoxy-benzamide;
689. N-{1-[6-oxo-4-(pyridine-2-sulfonyl)hexahydro-pyrrolo[3,2-b]pyrrole-1-carbonyl]-cyclohexyl}-4-thiophen-2-yl-benzamide;
690. 4-tert-Butyl-N-{1-cyclopropylmethyl-2-oxo-2-[6-oxo-4-(pyridine-2-sulfonyl)-hexahydro-pyrrolo[3,2-b]pyrrole-1-yl]-ethyl}-benzamide;
691. 5-Methoxy-benzofuran-2-carboxylic acid {1-cyclopropylmethyl-2-oxo-2-[6-oxo-4-(pyridine-2-sulfonyl)-hexahydro-pyrrolo[3,2-b]pyrrole-1-yl]-ethyl}-amide;
692. Thieno[3,2-b]thiophene-2-carboxylic acid {1-cyclopropylmethyl-2-oxo-2-[6-oxo-4-(pyridine-2-sulfonyl)-hexahydro-pyrrolo[3,2-b]pyrrol-1-yl]-ethyl}-amide;
693. 3-Methyl-benzofuran-2-carboxylic acid {1-cyclopropylmethyl-2-oxo-2-[6-oxo-4-(pyridine-2-sulfonyl)-hexahydro-pyrrolo[3,2-b]pyrrole-1-yl]-ethyl}-amide;
694. N-{1-cyclopropylmethyl-2-oxo-2-[6-oxo-4-(pyridine-2-sulfonyl)-hexahydro-pyrrolo[3,2-b]pyrrole-1-yl]-ethyl}-4-phenoxy-benzamide;
695. N-{1-cyclopropylmethyl-2-oxo-2-[6-oxo-4-(pyridine-2-sulfonyl)-hexahydro-pyrrolo[3,2-b]pyrrole-1-yl]-ethyl}-4-thiophen-2-yl-benzamide;
696. 4-tert-Butyl-N-{2-oxo-2-[6-oxo-4-(pyridine-2-sulfonyl)-hexahydro-pyrrolo[3,2-b]pyrrole-1-yl]-1-thiophen-2-ylmethyl-ethyl}-benzamide;
697. 5-Methoxy-benzofuran-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
698. Thieno[3,2-b]thiophene-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
699. 3-Methyl-benzofuran-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
700. N-{2-oxo-2-[6-oxo-4-(pyridine-2-sulfonyl)-hexahydro-pyrrolo[3,2-b]pyrrol-1-yl]-1-thiophen-2-ylmethyl-ethyl}-4-phenoxy-benzamide;
701. N-{2-oxo-2-[6-oxo-4-(pyridine-2-sulfonyl)-hexahydro-pyrrolo[3,2-b]pyrrol-1-yl]-1-thiophen-2-ylmethyl-ethyl}-4-thiophen-2-yl-benzamide;
702. 4-tert-Butyl-N-{1-[6-oxo-4-(pyridine-3-sulfonyl)-hexahydro-pyrrolo[3,2-b]pyrrole-1-carbonyl]-cyclohexyl}-benzamide;
703. 5-Methoxy-benzofuran-2-carboxylic acid {1-[6-oxo-4-(pyridine-3-sulfonyl)-hexahydro-pyrrolo[3,2-b]pyrrole-1-carbonyl]-cyclohexyl}-amide;
704. Thieno[3,2-b]thiophene-2-carboxylic acid {1-[6-oxo-4-(pyridine-3-sulfonyl)-hexahydro-pyrrolo[3,2-b]pyrrole-1-carbonyl]-cyclohexyl}-amide;
705. 3-Methyl-benzofuran-2-carboxylic acid {1-[6-oxo-4-(pyridine-3-sulfonyl)-hexahydro-pyrrolo[3,2-b]pyrrole-1-carbonyl]-cyclohexyl}-amide;
706. N-{1-[6-oxo-4-(pyridine-3-sulfonyl)-hexahydro-pyrrolo[3,2-b]pyrrole-1-carbonyl]-cyclohexyl}-4-phenoxy-benzamide;
707. N-{1-[6-oxo-4-(pyridine-3-sulfonyl)-hexahydro-pyrrolo[3,2-b]pyrrole-1-carbonyl]-cyclohexyl}-4-thiophen-2-yl-benzamide;
708. 4-tert-Butyl-N-{1-cyclopropylmethyl-2-oxo-2-[6-oxo-4-(pyridine-3-sulfonyl)-hexahydro-pyrrolo[3,2-b]pyrrole-1-yl]-ethyl}-benzamide;
709. 5-Methoxy-benzofuran-2-carboxylic acid {1-cyclopropylmethyl-2-oxo-2-[6-oxo-4-(pyridine-3-sulfonyl)-hexahydro-pyrrolo[3,2-b]pyrrole-1-yl]-ethyl}-amide;
710. Thieno[3,2-b]thiophene-2-carboxylic acid {1-cyclopropylmethyl-2-oxo-2-[6-oxo-4-(pyridine-3-sulfonyl)-hexahydro-pyrrolo[3,2-b]pyrrol-1-yl]-ethyl}-amide;
711. 3-Methyl-benzofuran-2-carboxylic acid {1-cyclopropylmethyl-2-oxo-2-[6-oxo-4-(pyridine-3-sulfonyl)-hexahydro-pyrrolo[3,2-b]pyrrole-1-yl]-ethyl}-amide;
712. N-{1-cyclopropylmethyl-2-oxo-2-[6-oxo-4-(pyridine-3-sulfonyl)-hexahydro-pyrrolo[3,2-b]pyrrole-1-yl]-ethyl}-4-phenoxy-benzamide;
713. N-{1-cyclopropylmethyl-2-oxo-2-[6-oxo-4-(pyridine-3-sulfonyl)-hexahydro-pyrrolo[3,2-b]pyrrole-1-yl]-ethyl}-4-thiophen-2-yl-benzamide;
714. 4-tert-Butyl-N-{2-oxo-2-[6-oxo-4-(pyridine-3-sulfonyl)-hexahydro-pyrrolo[3,2-b]pyrrole-1-yl]-1-thiophen-2-ylmethyl-ethyl}-benzamide;
715. 5-Methoxy-benzofuran-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
716. Thieno[3,2-b]thiophene-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
717. 3-Methyl-benzofuran-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
718. N-{2-oxo-2-[6-oxo-4-(pyridine-3-sulfonyl)-hexahydro-pyrrolo[3,2-b]pyrrol-1-yl]-1-thiophen-2-ylmethyl-ethyl}-4-phenoxy-benzamide;
719. N-{2-oxo-2-[6-oxo-4-(pyridine-3-sulfonyl)-hexahydro-pyrrolo[3,2-b]pyrrol-1-yl]-1-thiophen-2-ylmethyl-ethyl}-4-thiophen-2-yl-benzamide;
720. 4-tert-Butyl-N-{1-[6-oxo-4-(1-oxy-pyridine-2-sulfonyl)-hexahydro-pyrrolo[3,2-b]pyrrole-1-carbonyl]-cyclohexyl}-benzamide;
721. 5-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
722. Thieno[3,2-b]thiophene-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
723. 3-Methyl-benzofuran-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
724. N-{1-[6-oxo-4-(1-oxy-pyridine-2-sulfonyl)-hexahydro-pyrrolo[3,2-b]pyrrole-1-carbonyl]-cyclohexyl}-4-phenoxy-benzamide;
725. N-{1-[6-oxo-4-(1-oxy-pyridine-2-sulfonyl)-hexahydro-pyrrolo[3,2-b]pyrrole-1-carbonyl]-cyclohexyl}-4-thiophen-2-yl-benzamide;
726. 4-tert-Butyl-N-{1-cyclopropylmethyl-2-oxo-2-[6-oxo-(1-oxy-pyridine-2-sulfonyl)-hexahydro-pyrrolo[3,2-b]pyrrole-1-yl]-ethyl}-benzamide;
727. 5-Methoxy-benzofuran-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
728. Thieno[3,2-b]thiophene-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
729. 3-Methyl-benzofuran-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
730. N-{1-cyclopropylmethyl-2-oxo-2-[6-oxo-4-(1-oxy-pyridine-2-sulfonyl)-hexahydro-pyrrolo[3,2-b]pyrrole-1-yl]-ethyl}-4-phenoxy-benzamide;
731. N-{1-cyclopropylmethyl-2-oxo-2-[6-oxo-4-(1-oxy-pyridine-2-sulfonyl)-hexahydro-pyrrolo[3,2-b]pyrrole-1-yl]-ethyl}-4-thiophen-2-yl-benzamide;
732. 4-tert-Butyl-N-{2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-benzamide;
733. 5-Methoxy-benzofuran-2-carboxylic acid {2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-amide;
734. Thieno[3,2-b]thiophene-2-carboxylic acid {2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-amide;
735. 3-Methyl-benzofuran-2-carboxylic acid {2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-amide;
736. N-{2-oxo-2-[6-oxo-4-(1-oxy-pyridine-2-sulfonyl)-hexahydro-pyrrolo[3,2-b]pyrrol-1-yl]-1-thiophen-2-ylmethyl-ethyl}-4-phenoxy-benzamide;
737. N-{2-oxo-2-[6-oxo-4-(1-oxy-pyridine-2-sulfonyl)-hexahydro-pyrrolo[3,2-b]pyrrol-1-yl]-1-thiophen-2-ylmethyl-ethyl}-4-thiophen-2-yl-benzamide;
738. 4-tert-Butyl-N-{1-[6-oxo-4-(1-oxy-pyridine-3-sulfonyl)-hexahydro-pyrrolo[3,2-b]pyrrole-1-carbonyl]-cyclohexyl}-benzamide;
739. 5-Methoxy-benzofuran-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
740. Thieno[3,2-b]thiophene-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
741. 3-Methyl-benzofuran-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
742. N-{1-[6-oxo-4-(1-oxy-pyridine-3-sulfonyl)-hexahydro-pyrrolo[3,2-b]pyrrole-1-carbonyl]-cyclohexyl}-4-phenoxy-benzamide;
743. N-{1-[6-oxo-4-(1-oxy-pyridine-3-sulfonyl)-hexahydro-pyrrolo[3,2-b]pyrrole-1-carbonyl]-cyclohexyl}-4-thiophen-2-yl-benzamide;
744. 4-tert-Butyl-N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-benzamide;
745. 5-Methoxy-benzofuran-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
746. Thieno[3,2-b]thiophene-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
747. 3-Methyl-benzofuran-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
748. N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-4-phenoxy-benzamide;
749. N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-4-thiophen-2-yl-benzamide;
750. 4-tert-Butyl-N-{2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-benzamide;
751. 5-Methoxy-benzofuran-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
752. Thieno[3,2-b]thiophene-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
753. 3-Methyl-benzofuran-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
754. N-{2-oxo-2-[6-oxo-4-(1-oxy-pyridine-3-sulfonyl)-hexahydro-pyrrolo[3,2-b]pyrrol-1-yl]-1-thiophen-2-ylmethyl-ethyl}-4-phenoxy-benzamide;
755. N-{2-oxo-2-[6-oxo-4-(1-oxy-pyridine-3-sulfonyl)-hexahydro-pyrrolo[3,2-b]pyrrol-1-yl]-1-thiophen-2-ylmethyl-ethyl}-4-thiophen-2-yl-benzamide;
756. 4-tert-Butyl-N-{1-[6-oxo-4-(pyridine-2-carbonyl)-hexahydro-pyrrolo[3,2-b]pyrrole-1-carbonyl]-cyclohexyl}-benzamide;
757. 5-Methoxy-benzofuran-2-carboxylic acid {1-[6-oxo-4-(pyridine-2-carbonyl)-hexahydro-pyrrolo[3,2-b]pyrrole-1-carbonyl]-cyclohexyl}-amide;
758. Thieno[3,2-b]thiophene-2-carboxylic acid {1-[6-oxo-4-(pyridine-2-carbonyl)-hexahydro-pyrrolo[3,2-b]pyrrole-1-carbonyl]-cyclohexyl}-amide;
759. 3-Methyl-benzofuran-2-carboxylic acid {1-[6-oxo-4-(pyridine-2-carbonyl)-hexahydro-pyrrolo[3,2-b]pyrrole-1-carbonyl]-cyclohexyl}-amide;
760. N-{1-[6-oxo-4-(pyridine-2-carbonyl)-hexahydro-pyrrolo[3,2-b]pyrrole-1-carbonyl]-cyclohexyl})-phenoxy-benzamide;
761. N-{1-[6-oxo-4-(pyridine-2-carbonyl)-hexahydro-pyrrolo[3,2-b]pyrrole-1-carbonyl]-cyclohexyl}-4-thiophen-2-yl-benzamide;
762. 4-tert-Butyl-N-{1-cyclopropylmethyl-2-oxo-2-[6-oxo-4-(pyridine-2-carbonyl)-hexahydro-pyrrolo[3,2-b]pyrrole-1-yl]-ethyl}-benzamide;
763. 5-Methoxy-benzofuran-2-carboxylic acid {1-cyclopropylmethyl-2-oxo-2-[6-oxo-4-(pyridine-2-carbonyl)-hexahydro-pyrrolo[3,2-b]pyrrole-1-yl]-ethyl}-amide;
764. Thieno[3,2-b]thiophene-2-carboxylic acid {1-cyclopropylmethyl-2-oxo-2-[6-oxo-4-(pyridine-2-carbonyl)-hexahydro-pyrrolo[3,2-b]pyrrole-1-yl]-ethyl}-amide;
765. 3-Methyl-benzofuran-2-carboxylic acid {1-cyclopropylmethyl-2-oxo-2-[6-oxo-4-(pyridine-2-carbonyl) hexahydro-pyrrolo[3,2-b]pyrrole-1-yl]-ethyl}-amide;
766. N-{1-cyclopropylmethyl-2-oxo-2-[6-oxo-4-(pyridine-2-carbonyl)-hexahydro-pyrrolo[3,2-b]pyrrole-1-yl]-ethyl}-4-phenoxy-benzamide;
767. N-{1-cyclopropylmethyl-2-oxo-2-[6-oxo-4-(pyridine-2-carbonyl)-hexahydro-pyrrolo[3,2-b]pyrrole-1-yl]-ethyl}-4-thiophen-2-yl-benzamide;
768. 4-tert-Butyl-N-{2-oxo-2-[6-oxo-4-(pyridine-2-carbonyl)-hexahydro-pyrrolo[3,2-b]pyrrole-1-yl]-1-thiophen-2-ylmethyl-ethyl}-benzamide;
769. 5-Methoxy-benzofuran-2-carboxylic acid {2-oxo-2-[6-oxo-4-(pyridine-2-carbonyl)-hexahydro-pyrrolo[3,2-b]pyrrole-1-yl]-1-thiophen-2-ylmethyl-ethyl}-amide;
770. Thieno[3,2-b]thiophene-2-carboxylic acid {2-oxo-2-[6-oxo-4-(pyridine-2-carbonyl)-hexahydro-pyrrolo[3,2-b]pyrrole-1-yl]-1-thiophen-2-ylmethyl-ethyl}-amide;
771. 3-Methyl-benzofuran-2-carboxylic acid {2-oxo-2-[6-oxo-4-(pyridine-2-carbonyl)hexahydro-pyrrolo[3,2-b]pyrrole-1-yl]-1-thiophen-2-ylmethyl-ethyl}-amide;
772. N-{2-oxo-2-[6-oxo-4-(pyridine-2-carbonyl)-hexahydro-pyrrolo[3,2-b]pyrrol-1-yl]-1-thiophen-2-ylmethyl-ethyl}-4-phenoxy-benzamide;
773. N-{2-oxo-2-[6-oxo-4-(pyridine-2-carbonyl)hexahydro-pyrrolo[3,2-b]pyrrol-1-yl]-1-thiophen-2-ylmethyl-ethyl}-4-thiophen-2-yl-benzamide;
774. 4-tert-Butyl-N-{1-[6-oxo-4-(2-pyridin-3-yl-acetyl)-hexahydro-pyrrolo[3,2-b]pyrrole-1-carbonyl]-cyclohexyl}-benzamide;
775. 5-Methoxy-benzofuran-2-carboxylic acid {1-[6-oxo-4-(2-pyridin-3-yl-acetyl)-hexahydro-pyrrolo[3,2-b]pyrrole-1-carbonyl]-cyclohexyl}-amide;
776. Thieno[3,2-b]thiophene-2-carboxylic acid {1-[6-oxo-4-(2-pyridin-3-yl-acetyl)-hexahydro-pyrrolo[3,2-b]pyrrole-1-carbonyl]-cyclohexyl}-amide;
777. 3-Methyl-benzofuran-2-carboxylic acid {1-[6-oxo-4-(2-pyridin-3-yl-acetyl)-hexahydro-pyrrolo[3,2-b]pyrrole-1-carbonyl]-cyclohexyl}-amide;
778. N-{1-[6-oxo-4-(2-pyridin-3-yl-acetyl)-hexahydro-pyrrolo[3,2-b]pyrrole-1-carbonyl]-cyclohexyl}-4-phenoxy-benzamide;
779. N-{1-[6-oxo-4-(2-pyridin-3-yl-acetyl)-hexahydro-pyrrolo[3,2-b]pyrrole-1-carbonyl]-cyclohexyl}-4-thiophen-2-yl-benzamide;
780. 4-tert-Butyl-N-{1-cyclopropylmethyl-2-oxo-2-[6-oxo-4-(2-pyridin-3-yl-acetyl)-hexahydro-pyrrolo[3,2-b]pyrrole-1-yl]-ethyl}-benzamide;
781. 5-Methoxy-benzofuran-2-carboxylic acid {1-cyclopropylmethyl-2-oxo-2-[6-oxo-4-(2-pyridin-3-yl-acetyl)-hexahydro-pyrrolo[3,2-b]pyrrole-1-yl]-ethyl}-amide;
782. Thieno[3,2-b]thiophene-2-carboxylic acid {1-cyclopropylmethyl-2-oxo-2-[6-oxo-4-(2-pyridin-3-yl-acetyl)-hexahydro-pyrrolo[3,2-b]pyrrole-1-yl]-ethyl}-amide;
783. 3-Methyl-benzofuran-2-carboxylic acid {1-cyclopropylmethyl-2-oxo-2-[6-oxo-4-(2-pyridin-3-yl-acetyl)-hexahydro-pyrrolo[3,2-b]pyrrol-1-yl]-ethyl}-amide;
784. N-{1-cyclopropylmethyl-2-oxo-2-[6-oxo-4-(2-pyridin-3-yl-acetyl)-hexahydro-pyrrolo[3,2-b]pyrrole-1-yl]-ethyl}-4-phenoxy-benzamide;
785. N-{1-cyclopropylmethyl-2-oxo-2-[6-oxo-4-(2-pyridin-3-yl-acetyl)-hexahydro-pyrrolo[3,2-b]pyrrole-1-yl]-ethyl}-4-thiophen-2-yl-benzamide;
786. 4-tert-Butyl-N-{2-oxo-2-[6-oxo-4-(2-pyridin-3-yl-acetyl)-hexahydro-pyrrolo[3,2-b]pyrrole-1-yl]-1-thiophen-2-ylmethyl-ethyl}-benzamide;
787. 5-Methoxy-benzofuran-2-carboxylic acid {2-oxo-2-[6-oxo-4-(2-pyridin-3-yl-acetyl)-hexahydro-pyrrolo[3,2-b]pyrrole-1-yl]-1-thiophen-2-ylmethyl-ethyl}-amide;
788. Thieno[3,2-b]thiophene-2-carboxylic acid {2-oxo-2-[6-oxo-4-(2-pyridin-3-yl-acetyl)-hexahydro-pyrrolo[3,2-b]pyrrole-1-yl]-1-thiophen-2-ylmethyl-ethyl}-amide;
789. 3-Methyl-benzofuran-2-carboxylic acid {2-oxo-2-[6-oxo-4-(2-pyridin-3-yl-acetyl)-hexahydro-pyrrolo[3,2-b]pyrrol-1-yl]-1-thiophen-2-ylmethyl-ethyl}-amide;
790. N-{2-oxo-2-[6-oxo-4-(2-pyridin-3-yl-acetyl)-hexahydro-pyrrolo[3,2-b]pyrrol-1-yl]-1-thiophen-2-ylmethyl-ethyl}-4-phenoxy-benzamide;
791. N-{2-oxo-2-[6-oxo-4-(2-pyridin-3-yl-acetyl)-hexahydro-pyrrolo[3,2-b]pyrrol-1-yl]-1-thiophen-2-ylmethyl-ethyl}-4-thiophen-2-yl-benzamide;
792. 4-tert-Butyl-N-{1-[6-oxo-4-(pyridin-Z-ylmethanesulfonyl)-hexahydro-pyrrolo[3,2-b]pyrrole-1-carbonyl]-cyclohexyl}-benzamide;
793. 5-Methoxy-benzofuran-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
794. Thieno[3,2-b]thiophene-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
795. 3-Methyl-benzofuran-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
796. N-{1-[6-oxo-4-(pyridin-2-ylmethanesulfonyl)-hexahydro-pyrrolo[3,2-b]pyrrole-1-carbonyl]-cyclohexyl}-4-phenoxy-benzamide;
797. N-{1-[6-oxo-4-(pyridin-2-ylmethanesulfonyl)-hexahydro-pyrrolo[3,2-b]pyrrole-1-carbonyl]-cyclohexyl}-4-thiophen-2-yl-benzamide;
798. 4-tert-Butyl-N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-benzamide;
799. 5-Methoxy-benzofuran-2-carboxylic acid {1-cyclopropylmethyl-2-oxo-2-[6-oxo-4-(pyridin-2-ylmethanesulfonyl)-hexahydro-pyrrolo[3,2-b]pyrrol-1-yl]-ethyl}-amide;
800. Thieno[3,2-b]thiophene-2-carboxylic acid {1-cyclopropylmethyl-2-oxo-2-[6-oxo-4-(pyridin-2-ylmethanesulfonyl)-hexahydro-pyrrolo[3,2-b]pyrrol-1-yl]-ethyl}-amide;
801. 3-Methyl-benzofuran-2-carboxylic acid {1-cyclopropylmethyl-2-oxo-2-[6-oxo-4-(pyridin-2-ylmethanesulfonyl)-hexahydro-pyrrolo[3,2-b]pyrrole-1-yl]-ethyl}-amide;
802. N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-4-phenoxy-benzamide;
803. N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-4-thiophen-2-yl-benzamide;
804. 4-tert-Butyl-N-{2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-benzamide;
805. 5-Methoxy-benzofuran-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
806. Thieno[3,2-b]thiophene-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
807. 3-Methyl-benzofuran-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
808. N-{2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-4-phenoxy-benzamide;
809. N-{2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-4-thiophen-2-yl-benzamide;
810. 4-tert-Butyl-N-{1-[6-oxo-4-(pyridin-3-ylmethanesulfonyl)-hexahydro-pyrrolo[3,2-b]pyrrole-1-carbonyl]-cyclohexyl}-benzamide;
811. 5-Methoxy-benzofuran-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
812. Thieno[3,2-b]thiophene-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
813. 3-Methyl-benzofuran-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
814. N-{1-[6-oxo-4-(pyridin-3-ylmethanesulfonyl)-hexahydro-pyrrolo[3,2-b]pyrrole-1-carbonyl]-cyclohexyl}-4-phenoxy-benzamide;
815. N-{1-[6-oxo-4-(pyridin-3-ylmethanesulfonyl)-hexahydro-pyrrolo[3,2-b]pyrrole-1-carbonyl]-cyclohexyl}-4-thiophen-2-yl-benzamide;
816. 4-tert-Butyl-N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-benzamide;
817. 5-Methoxy-benzofuran-2-carboxylic acid {1-cyclopropylmethyl-2-oxo-2-[6-oxo-4-(pyridin-3-ylmethanesulfonyl)-hexahydro-pyrrolo[3,2-b]pyrrol-1-yl]-ethyl}-amide;
818. Thieno[3,2-b]thiophene-2-carboxylic acid {1-cyclopropylmethyl-2-oxo-2-[6-oxo-4-(pyridin-3-ylmethanesulfonyl)-hexahydro-pyrrolo[3,2-b]pyrrol-1-yl]-ethyl}-amide;
819. 3-Methyl-benzofuran-2-carboxylic acid {1-cyclopropylmethyl-2-oxo-2-[6-oxo-4-(pyridin-3-ylmethanesulfonyl)-hexahydro-pyrrolo[3,2-b]pyrrole-1-yl]-ethyl}-amide;
820. N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-4-phenoxy-benzamide;
821. N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-4-thiophen-2-yl-benzamide;
822. 4-tert-Butyl-N-{2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-benzamide;
823. 5-Methoxy-benzofuran-2-carboxylic acid {2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-amide;
824. Thieno[3,2-b]thiophene-2-carboxylic acid {2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-amide;
825. 3-Methyl-benzofuran-2-carboxylic acid {2-oxo-2-[6-oxo-4-(pyridin-3-ylmethanesulfonyl)-hexahydro-pyrrolo[3,2-b]pyrrol-1-yl]-1-thiophen-2-ylmethyl-ethyl}-amide;
826. N-{2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-4-phenoxy-benzamide;
827. N-{2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-4-thiophen-2-yl-benzamide;
828. 4-tert-Butyl-N-{1-[6-oxo-4-(1-oxy-pyridin-2-ylmethanesulfonyl)-hexahydro-pyrrolo[3,2-b]pyrrole-1-carbonyl]-cyclohexyl}-benzamide;
829. 5-Methoxy-benzofuran-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
830. Thieno[3,2-b]thiophene-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
831. 3-Methyl-benzofuran-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
832. N-{1-[6-oxo-4-(1-oxy-pyridin-2-ylmethanesulfonyl)-hexahydro-pyrrolo[3,2-b]pyrrole-1-carbonyl]-cyclohexyl}-4-phenoxy-benzamide;
833. N-{1-[6-oxo-4-(1-oxy-pyridin-2-ylmethanesulfonyl)-hexahydro-pyrrolo[3,2-b]pyrrole-1-carbonyl]-cyclohexyl}-4-thiophen-2-yl-benzamide;
834. 4-tert-Butyl-N-{1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-benzamide;
835. 5-Methoxy-benzofuran-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrol-1-yl]-ethyl}-amide;
836. Thieno[3,2-b]thiophene-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrol-1-yl]-ethyl}-amide;
837. 3-Methyl-benzofuran-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrol-1-yl]-ethyl}-amide;
838. N-{1-cyclopropylmethyl-2-oxo-2-[6-oxo-4-(1-oxy-pyridin-2-ylmethanesulfonyl)hexahydro-pyrrolo[3,2-b]pyrrole-1-yl]-ethyl}-4-phenoxy-benzamide;
839. N-{1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-4-thiophen-2-yl-benzamide;
840. 4-tert-Butyl-N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-benzamide;
841. 5-Methoxy-benzofuran-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
842. Thieno[3,2-b]thiophene-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
843. 3-Methyl-benzofuran-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
844. N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-4-phenoxy-benzamide;
845. N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-4-thiophen-2-yl-benzamide;
846. 4-tert-Butyl-N-{1-[6-oxo-4-(1-oxy-pyridin-3-ylmethanesulfonyl)-hexahydro-pyrrolo[3,2-b]pyrrole-1-carbonyl]-cyclohexyl}-benzamide;
847. 5-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
848. Thieno[3,2-b]thiophene-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
849. 3-Methyl-benzofuran-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
850. N-{1-[6-oxo-4-(1-oxy-pyridin-3-ylmethanesulfonyl)-hexahydro-pyrrolo[3,2-b]pyrrole-1-carbonyl]-cyclohexyl}-4-phenoxy-benzamide;
851. N-{1-[6-oxo-4-(1-oxy-pyridin-3-ylmethanesulfonyl)hexahydro-pyrrolo[3,2-b]pyrrole-1-carbonyl]-cyclohexyl}-4-thiophen-2-yl-benzamide;
852. 4-tert-Butyl-N-{1-cyclopropylmethyl-2-oxo-2-[6-oxo-4-(1-oxy-pyridin-3-ylmethanesulfonyl)hexahydro-pyrrolo[3,2-b]pyrrole-1-yl]-ethyl}-benzamide;
853. 5-Methoxy-benzofuran-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrol-1-yl]-ethyl}-amide;
854. Thieno[3,2-b]thiophene-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrol-1-yl]-ethyl}-amide;
855. 3-Methyl-benzofuran-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrol-1-yl]-ethyl}-amide;
856. N-{1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-4-phenoxy-benzamide;
857. N-{1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-4-thiophen-2-yl-benzamide;
858. 4-tert-Butyl-N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-benzamide;
859. 5-Methoxy-benzofuran-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
860. Thieno[3,2-b]thiophene-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
861. 3-Methyl-benzofuran-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
862. N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-4-phenoxy-benzamide;
863. N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-4-thiophen-2-yl-benzamide;
864. 2-Isobutyl-4-morpholin-4-yl-1-[6-oxo-4-(pyridine-2-sulfonyl)-hexahydro-pyrrolo[3,2-b]pyrrole-1-yl]-butane-1,4-one;
865. 2-Isobutyl-1-[6-oxo-4-(pyridine-2-sulfonyl)-hexahydro-pyrrolo[3,2-b]pyrrol-1-yl]-4-piperazin-1-yl-butane-1,4-dione;
866. 2-Isobutyl-1-(4-methyl-piperazin-1-yl)-1-[6-oxo-4-(1-pyridine-2-sulfonyl)-hexahydro-pyrrolo[3,2-b]pyrrole-1-yl]-butane-1,4-dione;
867. 2-Isobutyl-1-[6-oxo-4-(pyridine-2-sulfonyl)-hexahydro-pyrrolo[3,2-b]pyrrol-1-yl]-4-(4-phenyl-piperazin-1-yl)-butane-1,4-dione;
868. 2-Isobutyl-1-[6-oxo-4-(pyridine-2-sulfonyl)-hexahydro-pyrrolo[3,2-b]pyrrol-1-yl]-4-(3,4,4a,8a-tetrahydro-1H-isoquinolin-2-yl)-butane-1,4-dione;
869. 2-Isobutyl-1-[6-oxo-4-(pyridine-2-sulfonyl)-hexahydro-pyrrolo[3,2-b]pyrrol-1-yl]-4-(1,3,3a,7a-tetrahydro-isoindol-2-yl)-butane-1,4-dione;
870. 4-(4-Benzyl-piperazin-1-yl)-2-isobutyl-1-[6-oxo-4-(pyridine-2-sulfonyl)-hexahydro-pyrrolo[3,2-b]pyrrole-1-yl]-butane-1,4-dione;
871. 2-Isobutyl-4-morpholin-4-yl-1-[6-oxo-4-(pyridine-3-sulfonyl)-hexahydro-pyrrolo[3,2-b]pyrrole-1-yl]-butane-1,4-dione;
872. 2-Isobutyl-1-[6-oxo-4-(pyridine-3-sulfonyl)-hexahydro-pyrrolo[3,2-b]pyrrol-1-yl]-4-piperazin-1-yl-butane-1,4-dione;
873. 2-Isobutyl-4-(4-methyl-piperazin-1-yl)-1-[6-oxo-4-(pyridine-3-sulfonyl)-hexahydro-pyrrolo[3,2-b]pyrrole-1-yl]-butane-1,4-dione;
874. 2-Isobutyl-1-[6-oxo-4-(pyridine-3-sulfonyl)-hexahydro-pyrrolo[3,2-b]pyrrol-1-yl]-4-(4-phenyl-piperazin-1-yl)-butane-1,4-dione;
875. 2-Isobutyl-1-[6-oxo-4-(pyridine-3-sulfonyl)-hexahydro-pyrrolo[3,2-b]pyrrol-1-yl]-4-(3,4,4a,8a-tetrahydro-1H-isoquinolin-2-yl)-butane-1,4-dione;
876. 2-Isobutyl-1-[6-oxo-4-(pyridine-3-sulfonyl)-hexahydro-pyrrolo[3,2-b]pyrrol-1-yl]-4-(1,3,3a,7a-tetrahydro-isoindol-2-yl)-butane-1,4-dione;
877. 4-(4-Benzyl-piperazin-1-yl)-2-isobutyl-1-[6-oxo-4-(pyridine-3-sulfonyl)-hexahydro-pyrrolo[3,2-b]pyrrole-1-yl]-butane-1,4-dione;
878. 2-Isobutyl 4-morpholin-4-yl-1-[6-oxo-4-(1-oxy-pyridine-2-sulfonyl)-hexahydro-pyrrolo[3,2-b]pyrrole-1-yl]-butane-1,4-dione;
879. 2-Isobutyl-1-[6-oxo-4-(1-oxy-pyridine-2-sulfonyl)-hexahydro-pyrrolo[3,2-b]pyrrol-1-yl]-4-piperazin-1-yl-butane-1,4-dione;
880. 2-Isobutyl-4-(4-methyl-piperazin-1-yl)-1-[6-oxo-4-(1-oxy-pyridine-2-sulfonyl)-hexahydro-pyrrolo[3,2-b]pyrrole-1-yl]-butane-1,4-dione;
881. 2-Isobutyl-1-[6-oxo-4-(1-oxy-pyridine-2-sulfonyl)-hexahydro-pyrrolo[3,2-b]pyrrol-1-yl]-4-(4-phenyl-piperazin-1-yl)-butane-1,4-dione;
882. 2-Isobutyl-1-[6-oxo-4-(1-oxy-pyridine-2-sulfonyl)-hexahydro-pyrrolo[3,2-b]pyrrol-1-yl]-4-(3,4,4a,8a-tetrahydro-1H-isoquinolin-2-yl)-butane-1,4-dione;
883. 2-Isobutyl-1-[6-oxo-4-(1-oxy-pyridine-2-sulfonyl)-hexahydro-pyrrolo[3,2-b]pyrrol-1-yl]-4-(1,3,3a,7a-tetrahydro-isoindol-2-yl)-butane-1,4-dione;
884. 4-(4-Benzyl-piperazin-1-yl)₂-isobutyl-1-[6-oxo-4-(1-oxy-pyridine-2-sulfonyl)-hexahydro-pyrrolo[3,2-b]pyrrole-1-yl]-butane-1,4-dione;
885. 2-Isobutyl-4-morpholin-4-yl-1-[6-oxo-4-(1-oxy-pyridine-3-sulfonyl)-hexahydro-pyrrolo[3,2-b]pyrrole-1-yl]-butane-1,4-dione;
886. 2-Isobutyl-1-[6-oxo-4-(1-oxy-pyridine-3-sulfonyl)-hexahydro-pyrrolo[3,2-b]pyrrol-1-yl]-4-piperazin-1-yl-butane-1,4-dione;
887. 2-Isobutyl-4-(4-methyl-piperazin-1-yl)-1-[6-oxo-4-(1-oxy-pyridine-3-sulfonyl)-hexahydro-pyrrolo[3,2-b]pyrrole-1-yl]-butane-1,4-dione;
888. 2-Isobutyl-1-[6-oxo-4-(1-oxy-pyridine-3-sulfonyl)-hexahydro-pyrrolo[3,2-b]pyrrol-1-yl]-4-(4-phenyl-piperazin-1-yl)-butane-1,4-dione;
889. 2-Isobutyl-1-[6-oxo-4-(1-oxy-pyridine-3-sulfonyl)-hexahydro-pyrrolo[3,2-b]pyrrol-1-yl]-4-(3,4,4a,8a-tetrahydro-1H-isoquinolin-2-yl)-butane-1,4-dione;
890. 2-Isobutyl-1-[6-oxo-4-(1-oxy-pyridine-3-sulfonyl)-hexahydro-pyrrolo[3,2-b]pyrrol-1-yl]-4-(1,3,3a,7a-tetrahydro-isoindol-2-yl)-butane-1,4-dione;
891. 4-(4-Benzyl-piperazin-1-yl)-2-isobutyl-1-[6-oxo-4-(1-oxy-pyridine-3-sulfonyl)-hexahydro-pyrrolo[3,2-b]pyrrole-1-yl]-butane-1,4-dione;
892. 2-Isobutyl-4-morpholin-4-yl-1-[6-oxo-4-(pyridine-2-carbonyl)hexahydro-pyrrolo[3,2-b]pyrrole-1-yl]-butane-1,4-dione;
893. 2-Isobutyl-1-[6-oxo-4-(pyridine-2-carbonyl)-hexahydro-pyrrolo[3,2-b]pyrrol-1-yl]-4-piperazin-1-yl-butane-1,4-dione;
894. 2-Isobutyl-4-(4-methyl-piperazin-1-yl)-1-[6-oxo-4-(pyridine-2-carbonyl)-hexahydro-pyrrolo[3,2-b]pyrrole-1-yl]-butane-1,4-dione;
895. 2-Isobutyl-1-[6-oxo-4-(pyridine-2-carbonyl)-hexahydro-pyrrolo[3,2-b]pyrrol-1-yl]-4-(4-phenyl-piperazin-1-yl)-butane-1,4-dione;
896. 2-Isobutyl-1-[6-oxo-4-(pyridine-2-carbonyl)-hexahydro-pyrrolo[3,2-b]pyrrol-1-yl]-4-(3,4,4a,8a-tetrahydro-1H-isoquinolin-2-yl)-butane-1,4-dione;
897. 2-Isobutyl-1-[6-oxo-4-(pyridine-2-carbonyl)hexahydro-pyrrolo[3,2-b]pyrrol-1-yl]-4-(1,3,3a,7a-tetrahydro-isoindol-2-yl)-butane-1,4-dione;
898. 4-(4-Benzyl-piperazin-1-yl)-2-isobutyl-1-[6-oxo-4-(pyridine-2-carbonyl)-hexahydro-pyrrolo[3,2-b]pyrrole-1-yl]-butane-1,4-dione;
899. 2-Isobutyl-4-morpholin-4-yl-1-[6-oxo-4-(2-pyridin-3-yl-acetyl)-hexahydro-pyrrolo[3,2-b]pyrrole-1-yl]-butane-1,4-dione;
900. 2-Isobutyl-1-[6-oxo-4-(2-pyridin-3-yl-acetyl)-hexahydro-pyrrolo[3,2-b]pyrrol-1-yl]-4-piperazin-1-yl-butane-1,4-dione;
901. 2-Isobutyl-1-(4-methyl-piperazin-1-yl)-1-[6-oxo-4-(2-pyridin-3-yl-acetyl)-hexahydro-pyrrolo[3,2-b]pyrrole-1-yl]-butane-1,4-dione;
902. 2-Isobutyl-1-[6-oxo-4-(2-pyridin-3-yl-acetyl)-hexahydro-pyrrolo[3,2-b]pyrrol-1-yl]-4-(4-phenyl-piperazin-1-yl)-butane-1,4-dione;
903. 2-Isobutyl-1-[6-oxo-4-(2-pyridin-3-yl-acetyl)-hexahydro-pyrrolo[3,2-b]pyrrol-1-yl]-4-(3,4,4a,8a-tetrahydro-1H-isoquinolin-2-yl)-butane-1,4-dione;
904. 2-Isobutyl-1-[6-oxo-4-(2-pyridin-3-yl-acetyl)-hexahydro-pyrrolo[3,2-b]pyrrol-1-yl]-4-(1,3,3a,7a-tetrahydro-isoindol-2-yl)-butane-1,4-dione;
905. 4-(4-Benzyl-piperazin-1-yl)-2-isobutyl-1-[6-oxo-4-(2-pyridin-3-yl-acetyl)-hexahydro-pyrrolo[3,2-b]pyrrole-1-yl]-butane-1,4-dione;
906. 2-Isobutyl-4-morpholin-4-yl-1-[6-oxo-4-(pyridin-2-ylmethanesulfonyl)-hexahydro-pyrrolo[3,2-b]pyrrole-1-yl]-butane-1,4-dione;
907. 2-Isobutyl-1-[6-oxo-4-(pyridin-2-ylmethanesulfonyl)-hexahydro pyrrolo[3,2-b]pyrrol-1-yl]-4-piperazin-1-yl-butane-1,4-dione;
908. 2-Isobutyl-4-(4-methyl-piperazin-1-yl)-1-[6-oxo-4-(pyridin-2-ylmethanesulfonyl)-hexahydro-pyrrolo[3,2-b]pyrrole-1-yl]-butane-1,4-dione;
909. 2-Isobutyl-1-[6-oxo-4-(pyridin-2-ylmethanesulfonyl)-hexahydro-pyrrolo[3,2-b]pyrrole-1-yl]-4-(4-phenyl-piperazin-1-yl)-butane-1,4-dione;
910. 2-Isobutyl-1-[6-oxo-4-(pyridin-2-ylmethanesulfonyl-hexahydro-pyrrolo[3,2-b]pyrrole-1-yl]-4-(3,4,4a,8a-tetrahydro-1H-isoquinolin-2-yl)-butane-1,4-dione;
911. 2-Isobutyl-1-[6-oxo-4-(pyridin-2-ylmethanesulfonyl)-hexahydro-pyrrolo[3,2-b]pyrrole-1-yl]-4-(1,3,3a,7a-tetrahydro-isoindol-2-yl)-butane-1,4-dione;
912. 4-(4-Benzyl-piperazin-1-yl)-2-isobutyl-1-[6-oxo-4-(pyridin-2-ylmethanesulfonyl)-hexahydro-pyrrolo[3,2-b]pyrrole-1-yl]-butane-1,4-dione;
913. 2-Isobutyl-4-morpholin-4-yl-1-[6-oxo-4-(pyridin-3-ylmethanesulfonyl)-hexahydro-pyrrolo[3,2-b]pyrrole-1-yl]-butane-1,4-dione;
914. 2-Isobutyl-1-[6-oxo-4-(pyridin-3-ylmethanesulfonyl)-hexahydro-pyrrolo[3,2-b]pyrrole-1-yl]-4-piperazin-1-yl-butane-1,4-dione;
915. 2-Isobutyl-1-(4-methyl-piperazin-1-yl)-1-[6-oxo-4-(pyridin-3-ylmethanesulfonyl)-hexahydro-pyrrolo[3,2-b]pyrrole-1-yl]-butane-1,4-dione;
916. 2-Isobutyl-1-[6-oxo-4-(pyridin-3-ylmethanesulfonyl)-hexahydro-pyrrolo[3,2-b]pyrrole-1-yl]-4-(4-phenyl-piperazin-1-yl)-butane-1,4-dione;
917. 2-Isobutyl-1-[6-oxo-4-(pyridin-3-ylmethanesulfonyl)-hexahydro-pyrrolo[3,2-b]pyrrole-1-yl]-4-(3,4,4a,8a-tetrahydro-1H-isoquinolin-2-yl)-butane-1,4-dione;
918. 2-Isobutyl-1-[6-oxo-4-(pyridin-3-ylmethanesulfonyl)-hexahydro-pyrrolo[3,2-b]pyrrole-1-yl]-4-(1,3,3a,7a-tetrahydro-isoindol-2-yl)-butane-1,4-dione;
919. 4-(4-Benzyl-piperazin-1-yl)-2-isobutyl-1-[6-oxo-4-(pyridin-3-ylmethanesulfonyl)-hexahydro-pyrrolo[3,2-b]pyrrole-1-yl]-butane-1,4-dione;
920. 2-Isobutyl-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione;
921. 2-Isobutyl-1-[6-oxo-4-(1-oxy-pyridin-2-ylmethanesulfonyl)-hexahydro-pyrrolo[3,2-b]pyrrole-1-yl]-4-piperazin-1-yl-butane-1,4-dione;
922. 2-Isobutyl-4-(4-methyl-piperazin-1-yl)-1-[6-oxo-4-(1-oxy-pyridin-2-ylmethanesulfonyl)-hexahydro-pyrrolo[3,2-b]pyrrole-1-yl]-butane-1,4-dione;
923. 2-Isobutyl-1-[6-oxo-4-(1-oxy-pyridin-2-ylmethanesulfonyl)-hexahydro-pyrrolo[3,2-b]pyrrol-1-yl]-4-(4-phenyl-piperazin-1-yl)-butane-1,4-dione;
924. 2-Isobutyl-1-[6-oxo-4-(1-oxy-pyridin-2-ylmethanesulfonyl)-hexahydro-pyrrolo[3,2-b]pyrrole-1-yl]-4-(3,4,4a,8a-tetrahydro-1H-isoquinolin-2-yl)-butane-1,4-dione;
925. 2-Isobutyl-1-[6-oxo-4-(1-oxy-pyridin-2-ylmethanesulfonyl)-hexahydro-pyrrolo[3,2-b]pyrrole-1-yl]-4-(1,3,3a,7a-tetrahydro-isoindol-2-yl)-butane-1,4-dione;
926. 4-(4-Benzyl-piperazin-1-yl)-2-isobutyl-1-[6-oxo-4-(1-oxy-pyridin-2-ylmethanesulfonyl)-hexahydro-pyrrolo[3,2-b]pyrrole-1-yl]-butane-1,4-dione;
927. 2-Isobutyl-4-morpholin-4-yl-1-[6-oxo-4-(1-oxy-pyridin-3-ylmethanesulfonyl)-hexahydro-pyrrolo[3,2-b]pyrrole-1-yl]-butane-1,4-dione;
928. 2-Isobutyl-1-[6-oxo-4-(1-oxy-pyridin-3-ylmethanesulfonyl)-hexahydro-pyrrolo[3,2-b]pyrrole-1-yl]-4-piperazin-1-yl-butane-1,4-dione;
929. 2-Isobutyl-4-(4-methyl-piperazin-1-yl)-1-[6-oxo-4-(1-oxy-pyridin-3-ylmethanesulfonyl)-hexahydro-pyrrolo[3,2-b]pyrrole-1-yl]-butane-1,4-dione;
930. 2-Isobutyl-1-[6-oxo-4-(1-oxy-pyridin-3-ylmethanesulfonyl)-hexahydro-pyrrolo[3,2-b]pyrrole-1-yl]-4-(4-phenyl-piperazin-1-yl)-butane-1,4-dione;
931. 2-Isobutyl-1-[6-oxo-4-(1-oxy-pyridin-3-ylmethanesulfonyl)-hexahydro pyrrolo[3,2-b]pyrrole-1-yl]-4-(3,4,4a,8a-tetrahydro-1H-isoquinolin-2-yl)-butane-1,4-dione;
932. 2-Isobutyl-1-[6-oxo-4-(1-oxy-pyridin-3-ylmethanesulfonyl)-hexahydro-pyrrolo[3,2-b]pyrrole-1-yl]-1-(1,3,3a,7a-tetrahydro-isoindol-2-yl)-butane-1,4-dione;
933. 4-(4-Benzyl-piperazin-1-yl)-2-isobutyl-1-[6-oxo-4-(1-oxy-pyridin-3-ylmethanesulfonyl)-hexahydro-pyrrolo[3,2-b]pyrrole-1-yl]-butane-1,4-dione;
934. 4-(2-Biphenyl-3-yl-4-methyl-pentanoyl)-1-(pyridine-2-sulfonyl)-hexahydro-pyrrolo[3,2-b]pyrrol-3-one;
935. 4-(2-Biphenyl-3-yl-4-methyl-pentanoyl)-1-(pyridine-3-sulfonyl)-hexahydro-pyrrolo[3,2-b]pyrrol-3-one;
936. 4-(2-Biphenyl-3-yl-4-methyl-pentanoyl)-1-(1-oxy-pyridine-2-sulfonyl)-hexahydro-pyrrolo[3,2-b]pyrrol-3-one;
937. 4-(2-Biphenyl-3-ylmethyl-pentanoyl)-1-(1-oxy-pyridine-3-sulfonyl)-hexahydro-pyrrolo[3,2-b]pyrrol-3-one;
938. 4-(2-Biphenyl-3-ylmethyl-pentanoyl)-1-(pyridine-2-carbonyl)-hexahydro-pyrrolo[3,2-b]pyrrol-3-one;
939. 4-(2-Biphenyl-3-yl-4-methyl-pentanoyl)-1-(2-pyridin-3-yl-acetyl) hexahydro-pyrrolo[3,2-b]pyrrol-3-one;
940. 4-(2-Biphenyl-3-yl-4-methyl-pentanoyl)-1-(pyridin-2-ylmethanesulfonyl)-hexahydro-pyrrolo[3,2-b]pyrrol-3-one;
941. 4-2-Biphenyl-3-yl-4-methyl-pentanoyl)-1-(pyridin-3-ylmethanesulfonyl)-hexahydro-pyrrolo[3,2-b]pyrrol-3-one;
942. 4-(2-Biphenyl-3-yl-4-methyl-pentanoyl)-1-(1-oxy-pyridin-2-ylmethanesulfonyl)-hexahydro-pyrrolo[3,2-b]pyrrol-3-one;
943. 4-(2-Biphenyl-3-yl-4-methyl-pentanoyl)-1-(1-oxy-pyridin-3-ylmethanesulfonyl)hexahydro-pyrrolo[3,2-b]pyrrol-3-one;
944. 4-[4-Methyl-2-(3-pyridin-2-yl-phenyl)-pentanoyl]-1-(pyridine-2-sulfonyl)-hexahydro-pyrrolo[3,2-b]pyrrol-3-one;
945. 4-[4-Methyl-2-(3-pyridin-2-yl-phenyl)-pentanoyl]-1-(pyridine-3-sulfonyl)-hexahydro-pyrrolo[3,2-b]pyrrol-3-one;
946. 4-[4-Methyl-2-(3-pyridin-2-yl-phenyl)-pentanoyl]-1-(1-oxy-pyridine-2-sulfonyl)-hexahydro-pyrrolo[3,2-b]pyrrol-3-one;
947. 4-[4-Methyl-2-(3-pyridin-2-yl-phenyl)-pentanoyl]-1-(1-oxy-pyridine-3-sulfonyl)-hexahydro-pyrrolo[3,2-b]pyrrol-3-one,
948. 4-[4-Methyl-2-(3-pyridin-2-yl-phenyl)-pentanoyl]-1-(pyridine-2-carbonyl)-hexahydro-pyrrolo[3,2-b]pyrrol-3-one;
949. 4-[4-Methyl-2-(3-pyridin-2-yl-phenyl)-pentanoyl]-1-(2-pyridin-3-yl-acetyl)-hexahydro-pyrrolo[3,2-b]pyrrol-3-one;
950. 4-[4-Methyl-2-(3-pyridin-2-yl-phenyl)-pentanoyl]-1-(pyridin-2-ylmethanesulfonyl)-hexahydro-pyrrolo[3,2-b]pyrrol-3-one;
951. 4-[4-Methyl-2-(3-pyridin-2-yl-phenyl)-pentanoyl]-1-(pyridin-3-ylmethanesulfonyl)-hexahydro-pyrrolo[3,2-b]pyrrol-3-one;
952. 4-[4-Methyl-2-(3-pyridin-2-yl-phenyl)-pentanoyl]-1-(1-oxy-pyridin-2-ylmethanesulfonyl)-hexahydro-pyrrolo[3,2-b]pyrrol-3-one;
953. 4-[4-Methyl-2-(3-pyridin-2-yl-phenyl)-pentanoyl]-1-(1-oxy-pyridin-3-ylmethanesulfonyl)-hexahydro-pyrrolo[3,2-b]pyrrol-3-one;
954. 4-[4-Methyl-2-(3-pyridin-3-yl-phenyl)-pentanoyl]-1-(pyridine-2-sulfonyl)-hexahydro-pyrrolo[3,2-b]pyrrol-3-one;
955. 4-[4-Methyl-2-(3-pyridin-3-yl-phenyl)-pentanoyl]-1-(pyridine-3-sulfonyl)-hexahydro-pyrrolo[3,2-b]pyrrol-3-one;
956. 4-[4-Methyl-2-3-pyridin-3-yl-phenyl)-pentanoyl]-1-(1-oxy-pyridine-2-sulfonyl)-hexahydro-pyrrolo[3,2-b]pyrrol-3-one;
957. 4-[4-Methyl-2-(3-pyridin-3-yl-phenyl)-pentanoyl]-1-(1-oxy-pyridine-3-sulfonyl)-hexahydro-pyrrolo[3,2-b]pyrrol-3-one;
958. 4-[4-Methyl-2-(3-pyridin-3-yl-phenyl)-pentanoyl]-1-(pyridine-2-carbonyl)-hexahydro-pyrrolo[3,2-b]pyrrol-3-one;
959. 4-[4-Methyl-2-(3-pyridin-3-yl-phenyl)-pentanoyl]-1-(2-pyridin-3-yl-acetyl)-hexahydro-pyrrolo[3,2-b]pyrrol-3-one;
960. 4-[4-Methyl-2-(3-pyridin-3-yl-phenyl)-pentanoyl]-1-(pyridin-2-ylmethanesulfonyl)-hexahydro-pyrrolo[3,2-b]pyrrol-3-one;
961. 4-[4-Methyl-2-(3-pyridin-3-yl-phenyl)-pentanoyl]-1-(pyridin-3-ylmethanesulfonyl)-hexahydro-pyrrolo[3,2-b]pyrrol-3-one;
962. 4-[4-Methyl-2-(3-pyridin-3-yl-phenyl)-pentanoyl]-1-(1-oxy-pyridin-2-ylmethanesulfonyl)-hexahydro-pyrrolo[3,2-b]pyrrol-3-one;
963. 4-[4-Methyl-2-(3-pyridin-3-yl-phenyl)-pentanoyl]-1-(1-oxy-pyridin-3-ylmethanesulfonyl)-hexahydro-pyrrolo[3,2-b]pyrrol-3-one;
964. 4-[4-Methyl-2-(3-pyridin-4-yl-phenyl)-pentanoyl]-1-(pyridine-2-sulfonyl)-hexahydro-pyrrolo[3,2-b]pyrrol-3-one;
965. 4-[4-Methyl-2-(3-pyridin-4-yl-phenyl)-pentanoyl]-1-(pyridine-3-sulfonyl)-hexahydro-pyrrolo[3,2-b]pyrrol-3-one;
966. 4-[4-Methyl-2-(3-pyridin-4-yl-phenyl)-pentanoyl]-1-(1-oxy-pyridine-2-sulfonyl)-hexahydro-pyrrolo[3,2-b]pyrrol-3-one;
967. 4-[4-Methyl-2-(3-pyridinyl-phenyl)-pentanoyl]-1-(1-oxy-pyridine-3-sulfonyl)-hexahydro-pyrrolo[3,2-b]pyrrol-3-one;
968. 4-[4-Methyl-2-(3-pyridin-4-yl-phenyl)-pentanoyl]-1-(pyridine-2-carbonyl)-hexahydro-pyrrolo[3,2-b]pyrrol-3-one;
969. 4-[4-Methyl-2-(3-pyridin-4-yl-phenyl)-pentanoyl]-1-(2-pyridin-3-yl-acetyl)-hexahydro-pyrrolo[3,2-b]pyrrol-3-one;
970. 4-[4-Methyl-2-(3-pyridin-4-yl-phenyl)-pentanoyl]-1-(pyridin-2-ylmethanesulfonyl)hexahydro-pyrrolo[3,2-b]pyrrol-3-one;
971. 4-[4-Methyl-2-(3-pyridin-4-yl-phenyl)-pentanoyl]-1-(pyridin-3-ylmethanesulfonyl)-hexahydro-pyrrolo[3,2-b]pyrrol-3-one;
972. 4-[4-Methyl-2-(3-pyridin-4-yl-phenyl)-pentanoyl]-1-(1-oxy-pyridin-2-ylmethanesulfonyl)-hexahydro-pyrrolo[3,2-b]pyrrol-3-one;
973. 4-[4-Methyl-2-(3-pyridin-4-yl-phenyl)-pentanoyl]-1-(1-oxy-pyridin-3-ylmethanesulfonyl)-hexahydro-pyrrolo[3,2-b]pyrrol-3-one.
   Other compounds of the invention include, but are not limited to, the following examples where all 4 stereoisomeric combinations of the bicyclic ketone are included where P₂ is CH₂, i.e. (3aS,6aS), (3aR,6aS), (3aS,6aR), (3aR,6aR) and also included are the equivalent analogues where P₂ is O and NH. More preferred compounds consist of the cis-bicyclic isomers which, when P₂ is CH₂, are designated as (3aR,6aS) and (3aS,6aR) and also more preferred are the equivalent cis-bicyclic analogues where P₂ is O and NH.
974. Furan-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
975. Thiophene-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
976. Benzo[b]thiophene-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
977. Furan-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
978. Thiophene-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
979. Benzo[b]thiophene-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
980. Naphthalene-1-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
981. Quinoline-8-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
982. 4-tert-Butyl-N-{1-(4-hydroxy-benzyl)-2-oxo-2-[6-oxo-4-(pyridine-2-sulfonyl)-hexahydro-pyrrolo[3,2-b]pyrrole-1-yl]-ethyl}-benzamide;
983. 4-tert-Butyl-N-{1-(4-fluoro-benzyl)-2-oxo-2-[6-oxo-4-(pyridine-2-sulfonyl)-hexahydro-pyrrolo[3,2-b]pyrrole-1-yl]-ethyl}-benzamide;
984. 4-tert-Butyl-N-{1-(4-methoxy-benzyl)-2-oxo-2-[6-oxo-4-(pyridine-2-sulfonyl)-hexahydro-pyrrolo[3,2-b]pyrrole-1-yl]-ethyl}-benzamide;
985. 4-tert-Butyl-N-{1-(3-hydroxy-benzyl)-2-oxo-2-[6-oxo-4-(pyridine-2-sulfonyl)-hexahydro-pyrrolo[3,2-b]pyrrole-1-yl]-ethyl}-benzamide;
986. Biphenyl-4-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
987. Biphenyl-4-carboxylic acid {1-(4-fluoro-benzyl)-2-oxo-2-[6-oxo-4-(pyridine-2-sulfonyl)-hexahydro-pyrrolo[3,2-b]pyrrole-1-yl]-ethyl}-amide;
988. Biphenyl-4-carboxylic acid {1-(4-methoxy-benzyl)-2-oxo-2-[6-oxo-4-(pyridine-2-sulfonyl)-hexahydro-pyrrolo[3,2-b]pyrrole-1-yl]-ethyl}-amide;
989. Biphenyl-4-carboxylic acid {1-(3-hydroxy-benzyl)-2-oxo-2-[6-oxo-4-(pyridine-2-sulfonyl)-hexahydro-pyrrolo[3,2-b]pyrrole-1-yl]-ethyl}-amide;
990. Furan-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
991. Thiophene-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
992. Benzo[b]thiophene-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
993. Furan-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
994. Thiophene-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
995. Benzo[b]thiophene-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
996. Naphthalene-1-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
997. Quinoline-8-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
998. 4-tert-Butyl-N-{1-(4-hydroxy-benzyl)-2-oxo-2-[6-oxo-4-(pyridine-3-sulfonyl)hexahydro-pyrrolo[3,2-b]pyrrole-1-yl]-ethyl}-benzamide;
999. 4-tert-Butyl-N-{1-(4-fluoro-benzyl)-2-oxo-2-[6-oxo-4-(pyridine-3-sulfonyl)-hexahydro-pyrrolo[3,2-b]pyrrole-1-yl]-ethyl}-benzamide;
1000. 4-tert-Butyl-N-{1-(4-methoxy-benzyl)-2-oxo-2-[6-oxo-4-(pyridine-3-sulfonyl)-hexahydro-pyrrolo[3,2-b]pyrrole-1-yl]-ethyl}-benzamide;
1001. 4-tert-Butyl-N-{1-(3-hydroxy-benzyl)-2-oxo-2-[6-oxo-4-(pyridine-3-sulfonyl)-hexahydro-pyrrolo[3,2-b]pyrrole-1-yl]-ethyl}-benzamide;
1002. Biphenyl-4-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
1003. Biphenyl-4-carboxylic acid {1-(4-fluoro-benzyl)-2-oxo-2-[6-oxo-4-(pyridine-3-sulfonyl)-hexahydro-pyrrolo[3,2-b]pyrrole-1-yl]-ethyl}-amide;
1004. Biphenyl-carboxylic acid {1-(4-methoxy-benzyl)-2-oxo-2-[6-oxo-4-(pyridine-3-sulfonyl)-hexahydro-pyrrolo[3,2-b]pyrrole-1-yl]-ethyl}-amide;
1005. Biphenyl-4-carboxylic acid {1-(3-hydroxy-benzyl)-2-oxo-2-[6-oxo-4-(pyridine-3-sulfonyl)-hexahydro-pyrrolo[3,2-b]pyrrole-1-yl]-ethyl}-amide;
1006. Furan-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
1007. Thiophene-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
1008. Benzo[b]thiophene-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
1009. Furan-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
1010. Thiophene-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
1011. Benzo[b]thiophene-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
1012. Naphthalene-1-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
1013. Quinoline-8-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
1014. 4-tert-Butyl-N-{1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
1015. 4-tert-Butyl-N-{1-(4-fluoro-benzyl)-2-oxo-2-[6-oxo-4-(1-oxy-pyridine-2-sulfonyl)-hexahydro-pyrrolo[3,2-b]pyrrole-1-yl]-ethyl}-benzamide;
1016. 4-tert-Butyl-N-{1-(4-methoxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
1017. 4-tert-Butyl-N-{1-(3-hydroxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
1018. Biphenyl-4-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
1019. Biphenyl-4-carboxylic acid {1-(4-fluoro-benzyl)-2-oxo-2-[6-oxo-4-(1-oxy-pyridine-2-sulfonyl)-hexahydro-pyrrolo[3,2-b]pyrrole-1-yl]-ethyl}-amide;
1020. Biphenyl-4-carboxylic acid {1-(4-methoxy-benzyl)-2-oxo-2-[6-oxo-4-(1-oxy-pyridine-2-sulfonyl)-hexahydro-pyrrolo[3,2-b]pyrrole-1-yl]-ethyl}-amide;
1021. Biphenyl-4-carboxylic acid {1-(3-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
1022. Furan-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
1023. Thiophene-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
1024. Benzo[b]thiophene-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
1025. Furan-3-carboxylic acid {1-cyclohexylmethyl-2-oxo-2-[6-oxo-4-(1-oxy-pyridine-3-sulfonyl)-hexahydro-pyrrolo[3,2-b]pyrrol-1-yl]-ethyl}-amide;
1026. Thiophene-3-carboxylic acid {1-cyclohexylmethyl-2-oxo-2-[6-oxo-4-(1-oxy-pyridine-3-sulfonyl)-hexahydro-pyrrolo[3,2-b]pyrrole-1-yl]-ethyl}-amide;
1027. Benzo[b]thiophene-3-carboxylic acid {1-cyclohexylmethyl-2-oxo-2-[6-oxo-4-(1-oxy-pyridine-3-sulfonyl)-hexahydro-pyrrolo[3,2-b]pyrrole-1-yl]-ethyl}-amide;
1028. Naphthalene-1-carboxylic acid {1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
1029. Quinoline-8-carboxylic acid {1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
1030. 4-tert-Butyl-N-{1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-benzamide;
1031. 4-tert-Butyl-N-{1-(4-fluoro-benzyl)-2-oxo-2-[6-oxo-4-(1-oxy-pyridine-3-sulfonyl)-hexahydro-pyrrolo[3,2-b]pyrrole-1-yl]-ethyl}-benzamide;
1032. 4-tert-Butyl-N-{1-(4-methoxy-benzyl)-2-oxo-2-[6-oxo-4-(1-oxy-pyridine-3-sulfonyl)-hexahydro-pyrrolo[3,2-b]pyrrole-1-yl]-ethyl}-benzamide;
1033. 4-tert-Butyl-N-{1-(3-hydroxy-benzyl)-2-oxo-2-[6-oxo-4-(1-oxy-pyridine-3-sulfonyl)hexahydro-pyrrolo[3,2-b]pyrrole-1-yl]-ethyl}-benzamide;
1034. Biphenyl-4-carboxylic acid {1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
1035. Biphenyl-4-carboxylic acid {1-(4-fluoro-benzyl)-2-oxo-2-[6-oxo-4-(1-oxy-pyridine-3-sulfonyl)-hexahydro-pyrrolo[3,2-b]pyrrole-1-yl]-ethyl}-amide;
1036. Biphenyl-4-carboxylic acid {1-(4-methoxy-benzyl)-2-oxo-2-[6-oxo-4-(1-oxy-pyridine-3-sulfonyl)-hexahydro-pyrrolo[3,2-b]pyrrole-1-yl]-ethyl}-amide;
1037. Biphenyl-4-carboxylic acid {1-(3-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
1038. Furan-3-carboxylic acid {3,3-dimethyl-1-[6-oxo-4-(pyridine-2-carbonyl)-hexahydro-pyrrolo[3,2-b]pyrrole-1-carbonyl]-butyl}-amide;
1039. Thiophene-3-carboxylic acid {3,3-dimethyl-1-[6-oxo-4-(pyridine-2-carbonyl)hexahydro-pyrrolo[3,2-b]pyrrole-1-carbonyl]-butyl}-amide;
1040. Benzo[b]thiophene-3-carboxylic acid {3,3-dimethyl-1-[6-oxo-4-(pyridine-2-carbonyl)-hexahydro-pyrrolo[3,2-b]pyrrole-1-carbonyl]-butyl}-amide;
1041. Furan-3-carboxylic acid {1-cyclohexylmethyl-2-oxo-2-[6-oxo-4-(pyridine-2-carbonyl)-hexahydro-pyrrolo[3,2-b]pyrrol-1-yl]-ethyl}-amide;
1042. Thiophene-3-carboxylic acid {1-cyclohexylmethyl-2-oxo-2-[6-oxo-4-(pyridine-2-carbonyl)-hexahydro-pyrrolo[3,2-b]pyrrole-1-yl]-ethyl}-amide;
1043. Benzo[b]thiophene-3-caboxylic acid {1-cyclohexylmethyl-2-oxo-2-[6-oxo-4-(pyridine-2-carbonyl)-hexahydro-pyrrolo[3,2-b]pyrrol-1-yl]-ethyl}-amide;
1044. Naphthalene-1-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
1045. Quinoline-8-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
1046. 4-tert-Butyl-N-{1-(4-hydroxy-benzyl)-2-oxo-2-[6-oxo-4-(pyridine-2-carbonyl)-hexahydro-pyrrolo[3,2-b]pyrrole-1-yl]-ethyl}-benzamide;
1047. 4-tert-Butyl-N-{1-(4-fluoro-benzyl)-2-oxo-2-[6-oxo-4-(pyridine-2-carbonyl)-hexahydro-pyrrolo[3,2-b]pyrrole-1-yl]-ethyl}-benzamide;
1048. 4-tert-Butyl-N-{1-(4-methoxy-benzyl)-2-oxo-2-[6-oxo-4-(pyridine-2-carbonyl)-hexahydro-pyrrolo[3,2-b]pyrrole-1-yl]-ethyl}-benzamide;
1049. 4-tert-Butyl-N-{1-(3-hydroxy-benzyl)-2-oxo-2-[6-oxo-4-(pyridine-2-carbonyl)-hexahydro-pyrrolo[3,2-b]pyrrole-1-yl]-ethyl}-benzamide;
1050. Biphenyl-4-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
1051. Biphenyl-4-carboxylic acid {1-(4-fluoro-benzyl)-2-oxo-2-[6-oxo-4-(pyridine-2-carbonyl)-hexahydro-pyrrolo[3,2-b]pyrrole-1-yl]-ethyl}-amide;
1052. Biphenyl-4-carboxylic acid {1-(4-methoxy-benzyl)-2-oxo-2-[6-oxo-4-(pyridine-2-carbonyl)-hexahydro-pyrrolo[3,2-b]pyrrole-1-yl]-ethyl}-amide;
1053. Biphenyl-4-carboxylic acid {1-(3-hydroxy-benzyl)-2-oxo-2-[6-oxo-4-(pyridine-2-carbonyl)-hexahydro-pyrrolo[3,2-b]pyrrole-1-yl]-ethyl}-amide;
1054. Furan-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
1055. Thiophene-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
1056. Benzo[b]thiophene-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
1057. Furan-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
1058. Thiophene-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
1059. Benzo[b]thiophene-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
1060. Naphthalene-1-carboxylic acid {1-(4-hydroxy-benzyl)-2-oxo-2-[6-oxo-4-(2-pyridin-3-yl-acetyl)hexahydro-pyrrolo[3,2-b]pyrrole-1-yl]-ethyl}-amide;
1061. Quinoline-8-carboxylic acid {1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
1062. 4-tert-Butyl-N-{1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-benzamide;
1063. 4-ten-Butyl-N-{1-(4-fluoro-benzyl)-2-oxo-2-[6-oxo-4-(2-pyridin-3-yl-acetyl)-hexahydro-pyrrolo[3,2-b]pyrrole-1-yl]-ethyl}-benzamide;
1064. 4-tert-Butyl-N-{1-(4-methoxy-benzyl)-2-oxo-2-[6-oxo-4-(2-pyridin-3-yl-acetyl)-hexahydro-pyrrolo[3,2-b]pyrrole-1-yl]-ethyl}-benzamide;
1065. 4-tert-Butyl-N-{1-(3-hydroxy-benzyl)-2-oxo-2-[6-oxo-4-(2-pyridin-3-yl-acetyl)-hexahydro-pyrrolo[3,2-b]pyrrole-1-yl]-ethyl}-benzamide;
1066. Biphenyl-4-carboxylic acid {1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
1067. Biphenyl-4-carboxylic acid {1-(4-fluoro-benzyl)-2-oxo-2-[6-oxo-4-(2-pyridin-3-yl-acetyl)-hexahydro-pyrrolo[3,2-b]pyrrole-1-yl]-ethyl}-amide;
1068. Biphenyl-4-carboxylic acid {1-(4-methoxy-benzyl)-2-oxo-2-[6-oxo-4-(2-pyridin-3-yl-acetyl)-hexahydro-pyrrolo[3,2-b]pyrrole-1-yl]-ethyl}-amide;
1069. Biphenyl-4-carboxylic acid {1-(3-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
1070. Furan-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
1071. Thiophene-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
1072. Benzo[b]thiophene-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
1073. Furan-3-carboxylic acid {1-cyclohexylmethyl-2-oxo-2-[6-oxo-4-(pyridin-2-ylmethanesulfonyl-hexahydro-pyrrolo[3,2-b]pyrrole-1-yl]-ethyl}-amide;
1074. Thiophene-3 carboxylic acid {1-cyclohexylmethyl-2-oxo-2-[6-oxo-4-(pyridin-2-ylmethanesulfonyl)-hexahydro-pyrrolo[3,2-b]pyrrole-1-yl]-ethyl}-amide;
1075. Benzo[b]thiophene-3-carboxylic acid {1-cyclohexylmethyl-2-oxo-2-[6-oxo-4-(pyridin-2-ylmethanesulfonyl)-hexahydro-pyrrolo[3,2-b]pyrrole-1-yl]-ethyl}-amide;
1076. Naphthalene-1-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
1077. Quinoline-8-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
1078. 4-tert-Butyl-N-{1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-benzamide;
1079. 4-tert-Butyl-N-{1-(4-fluoro-benzyl)-2-oxo-2-[6-oxo-4-(pyridin-2-ylmethanesulfonyl)-hexahydro-pyrrolo[3,2-b]pyrrole-1-yl]-ethyl}-benzamide;
1080. 4-tert-Butyl-N-{1-(4-methoxy-benzyl)-2-oxo-2-[6-oxo-4-(pyridin-2-ylmethanesulfonyl)-hexahydro-pyrrolo[3,2-b]pyrrole-1-yl]-ethyl}-benzamide;
1081. 4-tert-Butyl-N-{1-(3-hydroxy-benzyl)-2-oxo-2-[6-oxo-4-(pyridin-2-ylmethanesulfonyl)hexahydro-pyrrolo[3,2-b]pyrrole-1-yl]-ethyl}-benzamide;
1082. Biphenyl-4-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
1083. Biphenyl-4-carboxylic acid {1-(4-fluoro-benzyl)-2-oxo-2-[6-oxo-4-(pyridin-2-ylmethanesulfonyl)-hexahydro-pyrrolo[3,2-b]pyrrole-1-yl]-ethyl}-amide;
1084. Biphenyl-4-carboxylic acid {1-(4-methoxy-benzyl)-2-oxo-2-[6-oxo-4-(pyridin-2-ylmethanesulfonyl)-hexahydro-pyrrolo[3,2-b]pyrrole-1-yl]-ethyl}-amide;
1085. Biphenyl-4-carboxylic acid {1-(3-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
1086. Furan-3-carboxylic acid {3,3-dimethyl-1-[6-oxo-4-(pyridin-3-ylmethanesulfonyl)-hexahydro-pyrrolo[3,2-b]pyrrole-1-carbonyl]-butyl}-amide;
1087. Thiophene-3-carboxylic acid {3,3-dimethyl-1-[6-oxo-4-pyridin-3-ylmethanesulfonyl)-hexahydro-pyrrolo[3,2-b]pyrrole-1-carbonyl]-butyl}-amide;
1088. Benzo[b]thiophene-3-carboxylic acid {3,3-dimethyl-1-[6-oxo-4-(pyridin-3-ylmethanesulfonyl)-hexahydro-pyrrolo[3,2-b]pyrrole-1-carbonyl]-butyl}-amide;
1089. Furan-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
1090. Thiophene-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
1091. Benzo[b]thiophene-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
1092. Naphthalene-1-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
1093. Quinoline-8-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
1094. 4-tert-Butyl-N-{1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
1095. 4-tert-Butyl-N-{1-(4-fluoro-benzyl)-2-oxo-2-[6-oxo-4-(pyridin-3-ylmethanesulfonyl)-hexahydro-pyrrolo[3,2-b]pyrrole-1-yl]-ethyl}-benzamide;
1096. 4-tert-Butyl-N-{1-(4-methoxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
1097. 4-tert-Butyl-N-{1-(3-hydroxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
1098. Biphenyl-4-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
1099. Biphenyl-4-carboxylic acid {1-(4-fluoro-benzyl)-2-oxo-2-[6-oxo-4-(pyridin-3-ylmethanesulfonyl)hexahydro-pyrrolo[3,2-b]pyrrole-1-yl]-ethyl}-amide;
1100. Biphenyl-4-carboxylic acid {1-(4-methoxy-benzyl)-2-oxo-2-[6-oxo-4-(pyridin-3-ylmethanesulfonyl)-hexahydro-pyrrolo[3,2-b]pyrrole-1-yl]-ethyl}-amide;
1101. Biphenyl-4-carboxylic acid {1-(3-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
1102. Furan-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
1103. Thiophene-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
1104. Benzo[b]thiophene-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
1105. Furan-3-carboxylic acid {1-cyclohexylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
1106. Thiophene-3-carboxylic acid {1-cyclohexylmethyl-2-oxo-2-[6-oxo-(1-oxy-pyridin-2-ylmethanesulfonyl)-hexahydro-pyrrolo[3,2-b]pyrrole-1-yl]-ethyl}-amide;
1107. Benzo[b]thiophene-3-carboxylic acid {1-cyclohexylmethyl-2-oxo-2-[6-oxo-4-(1-oxy-pyridin-2-ylmethanesulfonyl)-hexahydro-pyrrolo[3,2-b]pyrrol-1-yl]-ethyl}-amide;
1108. Naphthalene-1-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1109. Quinoline-8-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1110. 4-tert-Butyl-N-{1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
1111. 4-tert-Butyl-N-{1-(4-fluoro-benzyl)-2-oxo-2-[6-oxo-(1-oxy-pyridin-2-ylmethanesulfonyl)hexahydro-pyrrolo[3,2-b]pyrrole-1-yl]-ethyl}-benzamide;
1112. 4-tert-Butyl-N-{1-(4-methoxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
1113. 4-tert-Butyl-N-{1-(3-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
1114. Biphenyl-4-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1115. Biphenyl-4-carboxylic acid {1-(4-fluoro-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1116. Biphenyl-4-carboxylic acid {1-(4-methoxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1117. Biphenyl carboxylic acid {1-(3-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
1118. Furan-3-carboxylic acid {3,3-dimethyl-1-[6-oxo-4-(1-oxy-pyridin-3-ylmethanesulfonyl)-hexahydro-pyrrolo[3,2-b]pyrrole-1-carbonyl]-butyl}-amide;
1119. Thiophene-3-carboxylic acid {3,3-dimethyl-1-[6-oxo-4-(1-oxy-pyridin-3-ylmethanesulfonyl)-hexahydro-pyrrolo[3,2-b]pyrrole-1-carbonyl]-butyl}-amide;
1120. Benzo[b]thiophene-3-carboxylic acid {3,3-dimethyl-1-[6-oxo-4-(1-oxy-pyridin-3-ylmethanesulfonyl)-hexahydro-pyrrolo[3,2-b]pyrrole-1-carbonyl]-butyl}-amide;
1121. Furan-3-carboxylic acid {1-cyclohexylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
1122. Thiophene-3-carboxylic acid {1-cyclohexylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
1123. Benzo[b]thiophene-3-carboxylic acid {1-cyclohexylmethyl-2-oxo-2-[6-oxo-4-(1-oxy-pyridin-3-ylmethanesulfonyl)hexahydro-pyrrolo[3,2-b]pyrrol-1-yl]-ethyl}-amide;
1124. Naphthalene-1-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
1125. Quinoline-8-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
1126. 4-tert-Butyl-N-{1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
1127. 4-tert-Butyl-N-{1-(4-fluoro-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
1128. 4-tert-Butyl-N-{1-(4-methoxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
1129. 4-tert-Butyl-N-{1-(3-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
1130. Biphenyl-4-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
1131. Biphenyl-4-carboxylic acid {1-(4-fluoro-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
1132. Biphenyl-carboxylic acid {1-(4-methoxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide,
1133. Biphenyl-4-carboxylic acid {1-(3-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
1134. 2-Cyclohexylmethyl-4-morpholin-4-yl-1-[6-oxo-4-(pyridine-2-sulfonyl)-hexahydro-pyrrolo[3,2-b]pyrrole-1-yl]-butane-1,4-dione;
1135. 2-Cyclohexylmethyl-4-morpholin-4-yl-1-[6-oxo-4-(pyridine-3-sulfonyl)-hexahydro-pyrrolo[3,2-b]pyrrole-1-yl]-butane-1,4-dione;
1136. 2-Cyclohexylmethyl morpholin-4-yl-1-[6-oxo-4-(1-oxy-pyridine-2-sulfonyl)-hexahydro-pyrrolo[3,2-b]pyrrole-1-yl]-butane-1,4-dione;
1137. 2-Cyclohexylmethyl-4-morpholin-4-yl-1-[6-oxo-4-(1-oxy-pyridine-3-sulfonyl)-hexahydro-pyrrolo[3,2-b]pyrrole-1-yl]-butane-1,4-dione;
1138. 2-Cyclohexylmethyl-4-morpholin-4-yl-1-[6-oxo-4-(pyridine-2-carbonyl)-hexahydro-pyrrolo[3,2-b]pyrrole-1-yl]-butane-1,4-dione;
1139. 2-Cyclohexylmethyl-4-morpholin-4-yl-1-[6-oxo-4-(2-pyridin-3-yl-acetyl)-hexahydro-pyrrolo[3,2-b]pyrrole-1-yl]-butane-1,4-dione;
1140. 2-Cyclohexylmethyl-4-morpholin-4-yl-4-yl-1-[6-oxo-4-(pyridin-2-ylmethanesulfonyl)-hexahydro-pyrrolo[3,2-b]pyrrole-1-yl]-butane-1,4-dione;
1141. 2-Cyclohexylmethyl-4-morpholin-4-yl-1-[6-oxo-4-(pyridin-3-ylmethanesulfonyl)-hexahydro-pyrrolo[3,2-b]pyrrole-1-yl]-butane-1,4-dione;
1142. 2-Cyclohexylmethyl-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione;
1143. 2-Cyclohexylmethyl-4-morpholin-4-yl-1-[6-oxo-4-(1-oxy-pyridin-3-ylmethanesulfonyl)-hexahydro-pyrrolo[3,2-b]pyrrole-1-yl]-butane-1,4-dione;
1144. 2-(2,2-Dimethyl-propyl)-4-morpholin-4-yl-1-[6-oxo-4-(pyridine-2-sulfonyl)-hexahydro-pyrrolo[3,2-b]pyrrole-1-yl]-butane-1,4-dione;
1145. 2-(2,2-Dimethyl-propyl)-4-morpholin-4-yl-1-[6-oxo-4-(pyridine-3-sulfonyl)-hexahydro-pyrrolo[3,2-b]pyrrole-1-yl]-butane-1,4-dione;
1146. 2-(2,2-Dimethyl-propyl)-4-morpholin-4-yl-1-[6-oxo-4-(1-oxy-pyridine-2-sulfonyl)-hexahydro-pyrrolo[3,2-b]pyrrole-1-yl]-butane-1,4-dione;
1147. 2-(2,2-Dimethyl-propyl)-4-morpholin-4-yl-1-[6-oxo-4-(1-oxy-pyridine-3-sulfonyl)-hexahydro-pyrrolo[3,2-b]pyrrole-1-yl]-butane-1,4-dione;
1148. 2-(2,2-Dimethyl-propyl)-4-morpholin-4-yl-1-[6-oxo-4-(pyridine-2-carbonyl)-hexahydro-pyrrolo[3,2-b]pyrrole-1-yl]-butane-1,4-dione;
1149. 2-(2,2-Dimethyl-propyl)-4-morpholin-4-yl-1-[6-oxo-4-(2-pyridin-3-yl-acetyl)-hexahydro-pyrrolo[3,2-b]pyrrole-1-yl]-butane-1,4-dione;
1150. 2-(2,2-Dimethyl-propyl)-4-morpholin-4-yl-1-[6-oxo-4-(pyridin-2-ylmethanesulfonyl)-hexahydro-pyrrolo[3,2-b]pyrrole-1-yl]-butane-1,4-dione;
1151. 2-(2,2-Dimethyl-propyl)-4-morpholin-4-yl-1-[6-oxo-4-(pyridin-3-ylmethanesulfonyl)-hexahydro-pyrrolo[3,2-b]pyrrole-1-yl]-butane-1,4-dione;
1152. 2-(2,2-Dimethyl-propyl)-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione;
1153. 2-(2,2-Dimethyl-propyl)-4-morpholin-4-yl-1-[6-oxo-4-(1-oxy-pyridin-3-ylmethanesulfonyl)hexahydro-pyrrolo[3,2-b]pyrrole-1-yl]-butane-1,4-dione.

Additional compounds of the invention include, but are not limited to, the following examples where all 4 stereoisomeric combinations of the bicyclic ketone are included where P₂ is CH₂, i.e. (3aS,6aS), (3aR,6aS), (3aS,6aR), (3aR,6aR) and also included are the equivalent analogues where P₂ is O and NH. More preferred compounds consist of the cis-bicyclic isomers which, when P₂ is CH₂, are designated as (3aR,6aS) and (3aS,6aR) and also more preferred are the equivalent cis-bicyclic analogues where P₂ is O and NH.
1154. N-[1-(4-Benzoyl-6-oxo-hexahydro-pyrrolo[3,2-b]pyrrole-1-carbonyl)-3-methyl-butyl]-4-tert-butylbenzamide;
1155. 4-tert-Butyl-N-{3-methyl-1-[6-oxo-4-(1-oxy-pyridine-2-carbonyl)-hexahydro-pyrrolo[3,2-b]pyrrole-1-carbonyl]-butyl}-benzamide;
1156. 4-tert-Butyl-N-{3-methyl-1-[6-oxo-4-(pyridine-3-carbonyl)-hexahydro-pyrrolo[3,2-b]pyrrole-1-carbonyl]-butyl}-benzamide;
1157. 4-tert-Butyl-N-{3-methyl-1-[6-oxo-4-(1-oxy-pyridine-3-carbonyl)-hexahydro-pyrrolo[3,2-b]pyrrole-1-carbonyl]-butyl}-benzamide;
1158. 4-tert-Butyl-N-{3-methyl-1-[6-oxo-4-(pyridine-4-carbonyl)-hexahydro-pyrrolo[3,2-b]pyrrole-1-carbonyl]-butyl}-benzamide;
1159. 4-tert-Butyl-N-{3-methyl-1-[6-oxo-4-(1-oxy-pyridinecarbonyl)-hexahydro-pyrrolo[3,2-b]pyrrole-1-carbonyl]-butyl}-benzamide;
1160. N-[1-(4-Benzenesulfonyl-6-oxo-hexahydro-pyrrolo[3,2-b]pyrrole-1-carbonyl)-3-methyl-butyl]-4-tert-butyl-benzamide;
1161. 4-tert-Butyl-N-{3-methyl-1-[6-oxo-4-(pyridine-sulfonyl)-hexahydro-pyrrolo[3,2-b]pyrrole-1-carbonyl]-butyl}-benzamide;
1162. 4-tert-Butyl-N-{3-methyl-1-[6-oxo-4-(1-oxy-pyridine-sulfonyl) hexahydro-pyrrolo[3,2-b]pyrrole-1-carbonyl]-butyl}-benzamide;
1163. 4-tert-Butyl-N-[3-methyl-1-(6-oxo-4-phenylacetyl-hexahydro-pyrrolo[3,2-b]pyrrole-1-carbonyl)-butyl]-benzamide;
1164. 4-[2-(4-tert-Butyl-benzoylamino)-4-methyl-pentanoyl]-3-oxo-hexahydro-pyrrolo[3,2-b]pyrrole-1-carboxylic acid phenylamide;
1165. 4-[2-(4-tert-Butyl-benzoylamino)-4-methyl-pentanoyl]-3-oxo-hexahydro-pyrrolo[3,2-b]pyrrole-1-carboxylic acid isobutyl-amide;
1166. 4-[2-(4-tert-Butyl-benzoylamino)-4-methyl-pentanoyl]-3-oxo-hexahydro-pyrrolo[3,2-b]pyrrole-1-carboxylic acid phenyl ester,
1167. 4-[2-(4-tert-Butyl-benzoylamino)-4-methyl-pentanoyl]-3-oxo-hexahydro-pyrrolo[3,2-b]pyrrole-1-carboxylic acid isobutyl ester,
1168. 4-tert-Butyl-N-{3-methyl-1-[6-oxo-4-(pyrrolidine-1-carbonyl)-hexahydro-pyrrolo[3,2-b]pyrrole-1-carbonyl]-butyl}-benzamide;
1169. 4-tert-Butyl-N-{3-methyl-1-[6-oxo-4-(piperidine-1-carbonyl)-hexahydro-pyrrolo[3,2-b]pyrrole-1-carbonyl]-butyl}-benzamide;
1170. 4-tert-Butyl-N-{3-methyl-1-[4-(morpholin-4-carbonyl)-6-oxo-hexahydro-pyrrolo[3,2-b]pyrrole-1-carbonyl]-butyl}-benzamide;
1171. 4-tert-Butyl-N-{3-methyl-1-[6-oxo-4-piperazine-1-carbonyl)-hexahydro-pyrrolo[3,2-b]pyrrole-1-carbonyl]-butyl}-benzamide;
1172. 4-tert-Butyl-N-{3-methyl-1-[4-(4-methyl-piperazine-1-carbonyl)-6-oxo-hexahydro-pyrrolo[3,2-b]pyrrole-1-carbonyl]-butyl}-benzamide;
1173. 4-tert-Butyl-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
1174. 4-tert-Butyl-N-{3-methyl-1-[6-oxo-4-(2-piperazin-1-yl-acetyl)-hexahydro-pyrrolo[3,2-b]pyrrole-1-carbonyl]-butyl}-benzamide;
1175. 4-tert-Butyl-N-{1-[4-(furan-2-carbonyl)-6-oxo-hexahydro-pyrrolo[3,2-b]pyrrole-1-carbonyl]-3-methyl-butyl}-benzamide;
1176. 4-tert-Butyl-N-{1-[4-(5-chloro-furan-2-carbonyl)-6-oxo-hexahydro-pyrrolo[3,2-b]pyrrole-1-carbonyl]-3-methyl-butyl}-benzamide;
1177. 4-tert-Butyl-N-{1-[4-(thiophene-2-carbonyl)-6-oxo-hexahydro-pyrrolo[3,2-b]pyrrole-1-carbonyl]-3-methyl-butyl}-benzamide;
1178. 4-tert-Butyl-N-{1-[4-(5-chloro-thiophene-2-carbonyl)-6-oxo-hexahydro-pyrrolo[3,2-b]pyrrole-1-carbonyl]-3-methyl-butyl}-benzamide;
1179. 4-tert-Butyl-N-{1-[4-(furan-3-carbonyl)-6-oxo-hexahydro-pyrrolo[3,2-b]pyrrole-1-carbonyl]-3-methyl-butyl}-benzamide;
1180. 4-tert-Butyl-N-{1-[4-(thiophene-3-carbonyl)-6-oxo-hexahydro-pyrrolo[3,2-b]pyrrole-1-carbonyl]-3-methyl-butyl}-benzamide;
1181. 4-tert-Butyl-N-[1-(4-cyclopentanecarbonyl-6-oxo-hexahydro-pyrrolo[3,2-b]pyrrole-1-carbonyl)-3-methyl-butyl]-benzamide;
1182. 4-tert-Butyl-N-[1-(4-cyclohexanecarbonyl-6-oxo-hexahydro-pyrrolo[3,2-b]pyrrole-1-carbonyl)-3-methyl-butyl]-benzamide;
1183. 4-tert-Butyl-N-{1-[4-(cyclopent-3-enecarbonyl)-6-oxo-hexahydro-pyrrolo[3,2-b]pyrrole-1-carbonyl]-3-methyl-butyl}-benzamide;
1184. 4-tert-Butyl-N-{1-[4-(2-cyclopentyl-acetyl)-6-oxo-hexahydro-pyrrolo[3,2-b]pyrrole-1-carbonyl]-3-methyl-butyl}-benzamide;
1185. 4-tert-Butyl-N-{1-[4-(2-cyclohexyl-acetyl)-6-oxo-hexahydro-pyrrolo[3,2-b]pyrrole-1-carbonyl]-3-methyl-butyl}-benzamide;
1186. 4-tert-Butyl-N-{3-methyl-1-[6-oxo-4-(pyrrolidine-2-carbonyl)-hexahydro-pyrrolo[3,2-b]pyrrole-1-carbonyl]-butyl}-benzamide;
1187. N-{1-[4-(1-Acetyl-pyrrolidine-2-carbonyl)-6-oxo-hexahydro-pyrrolo[3,2-b]pyrrole-1-carbonyl]-3-methyl-butyl}-4-tert-butyl-benzamide;
1188. 4-tert-Butyl-N-{3-methyl-1-[6-oxo-4-(piperidine-2-carbonyl)-hexahydro-pyrrolo[3,2-b]pyrrole-1-carbonyl]-butyl}-benzamide;
1189. N-{1-[4-(1-Acetyl-piperidine-2-carbonyl)-6-oxo-hexahydro-pyrrolo[3,2-b]pyrrole-1-carbonyl]-3-methyl-butyl}-4-tert-butyl-benzamide;
1190. 4-tert-Butyl-N-{3-methyl-1-[6-oxo-4-(tetrahydro-furan-2-carbonyl)-hexahydro-pyrrolo[3,2-b]pyrrole-1-carbonyl]-butyl}-benzamide;
1191. N-{1-[4-(1-Amino-cyclobutanecarbonyl)-6-oxo-hexahydro-pyrrolo[3,2-b]pyrrole-1-carbonyl]-3-methyl-butyl}-4-tert-butyl-benzamide;
1192. N-{1-[4-(1-Amino-cyclopentanecarbonyl)-6-oxo-hexahydro-pyrrolo[3,2-b]pyrrole-1-carbonyl]-3-methyl-butyl}-4-tert-butyl-benzamide;
1193. N-{1-[4-(1-Amino-cyclohexanecarbonyl)-6-oxo-hexahydro-pyrrolo[3,2-b]pyrrole-1-carbonyl]-3-methyl-butyl}-4-tert-butyl-benzamide;
1194. N-{1-[4-(1-Acetylamino-cyclobutanecarbonyl)-6-oxo-hexahydro-pyrrolo[3,2-b]pyrrole-1-carbonyl]-3-methyl-butyl}-4-tert-butyl-benzamide;
1195. N-{1-[4-(1-Acetylamino-cyclopentanecarbonyl)-6-oxo-hexahydro-pyrrolo[3,2-b]pyrrole-1-carbonyl]-3-methyl-butyl}-4-tert-butyl-benzamide;
1196. N-{1-[4-(1-Acetylamino-cyclohexanecarbonyl)-6-oxo-hexahydro-pyrrolo[3,2-b]pyrrole-1-carbonyl]-3-methyl-butyl}-4-tert-butyl-benzamide;
1197. 4-tert-Butyl-N-{1-[4-(1-hydroxy-cyclobutanecarbonyl)-6-oxo-hexahydro-pyrrolo[3,2-b]pyrrole-1-carbonyl]-3-methyl-butyl}-benzamide;
1198. 4-tert-Butyl-N-{1-[4-(1-hydroxy-cyclopentanecarbonyl)-6-oxo-hexahydro-pyrrolo[3,2-b]pyrrole-1-carbonyl]-3-methyl-butyl}-benzamide;
1199. 4-tert-Butyl-N-{1-[4-(1-hydroxy-cyclohexanecarbonyl)-6-oxo-hexahydro-pyrrolo[3,2-b]pyrrole-1-carbonyl]-3-methyl-butyl}-benzamide;
1200. 4-tert-Butyl-N-{1-[4-(1-methoxy-cyclobutanecarbonyl)-6-oxo-hexahydro-pyrrolo[3,2-b]pyrrole-1-carbonyl]-3-methyl-butyl}-benzamide;
1201. 4-tert-Butyl-N-{1-[4-(1-methoxy-cyclopentanecarbonyl)-6-oxo-hexahydro-pyrrolo[3,2-b]pyrrole-1-carbonyl]-3-methyl-butyl}-benzamide;
1202. 4-tert-Butyl-N-{1-[4-(1-methoxy-cyclohexanecarbonyl)-6-oxo-hexahydro-pyrrolo[3,2-b]pyrrole-1-carbonyl]-3-methyl-butyl}-benzamide;
1203. N-{1-[4-(2-Amino-2-cyclopentyl-acetyl)-6-oxo-hexahydro-pyrrolo[3,2-b]pyrrole-1-carbonyl]-3-methyl-butyl}-4-tert-butyl-benzamide;
1204. N-{1-[4-(2-Amino-2-cyclohexyl-acetyl)-6-oxo-hexahydro-pyrrolo[3,2-b]pyrrole-1-carbonyl]-3-methyl-butyl}-4-tert-butyl-benzamide;
1205. N-{1-[4-(2-Acetylamino-2-cyclopentyl-acetyl)-6-oxo-hexahydro-pyrrolo[3,2-b]pyrrole-1-carbonyl]-3-methyl-butyl}-4-tert-butyl-benzamide;
1206. N-{1-[4-(2-Acetylamino-2-cyclohexyl-acetyl)-6-oxo-hexahydro-pyrrolo[3,2-b]pyrrole-1-carbonyl]-3-methyl-butyl}-4-tert-butyl-benzamide;
1207. N-{1-[4-(2-Acetylamino-propionyl)-6-oxo-hexahydro-pyrrolo[3,2-b]pyrrole-1-carbonyl]-3-methyl-butyl}-4-tert-butyl-benzamide;
1208. N-{1-[4-(2-Acetylamino-acetyl)-6-oxo-hexahydro-pyrrolo[3,2-b]pyrrole-1-carbonyl]-3-methyl-butyl}-4-tert-butyl-benzamide;
1209. N-(1-{4-[2-(Acetyl-methyl-amino)-acetyl]-6-oxo-hexahydro-pyrrolo[3,2-b]pyrrole-carbonyl}-3-methyl-butyl)-4-tert-butyl-benzamide;
1210. 4-tert-Butyl-N-{1-[4-(2-dimethylamino-acetyl)-6-oxo-hexahydro-pyrrolo[3,2-b]pyrrole-1-carbonyl]-3-methyl-butyl}-benzamide;
1211. N-{1-[4-(2-Amino-butyryl)-6-oxo-hexahydro-pyrrolo[3,2-b]pyrrole-1-carbonyl]-3-methyl-butyl}-4-tert-butyl-benzamide;
1212. N-{1-[4-(2-Acetylamino-butyryl)-6-oxo-hexahydro-pyrrolo[3,2-b]pyrrole-1-carbonyl]-3-methyl-butyl}-4-tert-butyl-benzamide;
1213. N-{1-[4-(2-amino-3-methyl-butyryl)-6-oxo-hexahydro-pyrrolo[3,2-b]pyrrole-1-carbonyl]-3-methyl-butyl}-4-tert-butyl-benzamide;
1214. N-{1-[4-(2-Acetylamino-3-methyl-butyryl)-6-oxo-hexahydro-pyrrolo[3,2-b]pyrrole-1-carbonyl]-3-methyl-butyl}-4-tert-butyl-benzamide;
1215. N-{1-[4-(2-amino-3,3-dimethyl-butyryl)-6-oxo-hexahydro-pyrrolo[3,2-b]pyrrole-1-carbonyl]-3-methyl-butyl}-4-tert-butyl-benzamide;
1216. N-{1-[4-(2-Acetylamino-3,3-dimethyl-butyryl)-6-oxo-hexahydro-pyrrolo[3,2-b]pyrrole-1-carbonyl]-3-methyl-butyl}-4-tert-butyl-benzamide;
1217. N-{1-[4-(2-Amino-3-methyl-pentanoyl)-6-oxo-hexahydro-pyrrolo[3,2-b]pyrrole-1-carbonyl]-3-methyl-butyl}-4-tert-butyl-benzamide;
1218. N-{1-[4-(2-Acetylamino-3-methyl-pentanoyl)-6-oxo-hexahydro-pyrrolo[3,2-b]pyrrole-1-carbonyl]-3-methyl-butyl}-4-tert-butyl-benzamide;
1219. 4-tert-Butyl-N-[3-methyl-1-(6-oxo-4-pentanoyl-hexahydro-pyrrolo[3,2-b]pyrrole-1-carbonyl)-butyl]-benzamide;
1220. 4-tert-Butyl-N-{3-methyl-1-[4-(3-methyl-pentanoyl)-6-oxo-hexahydro-pyrrolo[3,2-b]pyrrole-1-carbonyl]-butyl}-benzamide;
1221. 4-tert-Butyl-N-{3-methyl-1-[4-(4-methyl-pentanoyl)-6-oxo-hexahydro-pyrrolo[3,2-b]pyrrole-1-carbonyl]-butyl}-benzamide;
1222. N-{1-[4-(2-Amino-4-methyl-pentanoyl)-6-oxo-hexahydro-pyrrolo[3,2-b]pyrrole-1-carbonyl]-3-methyl-butyl}-4-tert-butyl-benzamide;
1223. N-{1-[4-(2-Acetylamino-4-methyl-pentanoyl)-6-oxo-hexahydro-pyrrolo[3,2-b]pyrrole-1-carbonyl]-3-methyl-butyl}-4-tert-butyl-benzamide;
1224. N-{1-[4-(2-Amino-4,4-dimethyl-pentanoyl)-6-oxo-hexahydro-pyrrolo[3,2-b]pyrrole-1-carbonyl]-3-methyl-butyl}-4-tert-butyl-benzamide;
1225. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-tert-butyl-benzamide;
1226. N-{1-[4-(2-Amino-pentanoyl)-6-oxo-hexahydro-pyrrolo[3,2-b]pyrrole-1-carbonyl]-3-methyl-butyl}-4-tert-butyl-benzamide;
1227. N-{1-[4-(2-Acetylamino-pentanoyl)-6-oxo-hexahydro-pyrrolo[3,2-b]pyrrole-1-carbonyl]-3-methyl-butyl}-4-tert-butyl-benzamide;
1228. N-{1-[4-(2-Amino-5-methyl-hexanoyl)-6-oxo-hexahydro-pyrrolo[3,2-b]pyrrole-1-carbonyl]-3-methyl-butyl}-4-tert-butyl-benzamide;
1229. N-{1-[4-(2-Acetylamino-5-methyl-hexanoyl)-6-oxo-hexahydro-pyrrolo[3,2-b]pyrrole-1-carbonyl]-3-methyl-butyl}-4-tert-butyl-benzamide;
1230. N-{1-[4-(2-hydroxy-3-methyl-butyryl)-6-oxo-hexahydro-pyrrolo[3,2-b]pyrrole-1-carbonyl]-3-methyl-butyl}-4-tert-butyl-benzamide;
1231. N-{1-[4-(2-methoxy-3-methyl-butyryl)-6-oxo-hexahydro-pyrrolo[3,2-b]pyrrole-1-carbonyl]-3-methyl-butyl}-4-tert-butyl-benzamide;
1232. 4-tert-Butyl-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
1233. 4-tert-Butyl-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
1234. 4-tert-Butyl-N-{1-[4-(2-hydroxy-3-methyl-pentanoyl)-6-oxo-hexahydro-pyrrolo[3,2-b]pyrrole-1-carbonyl]-3-methyl-butyl}-benzamide;
1235. 4-tert-Butyl-N-{1-[4-(2-methoxy-3-methyl-pentanoyl)-6-oxo-hexahydro-pyrrolo[3,2-b]pyrrole-1-carbonyl]-3-methyl-butyl}-benzamide;
1236. 4-tert-Butyl-N-{1-[4-(2-hydroxy-4-methyl-pentanoyl)-6-oxo-hexahydro-pyrrolo[3,2-b]pyrrole-1-carbonyl]-3-methyl-butyl}-benzamide;
1237. 4-tert-Butyl-N-{1-[4-(2-methoxy-4-methyl-pentanoyl)-6-oxo-hexahydro-pyrrolo[3,2-b]pyrrole-1-carbonyl]-3-methyl-butyl}-benzamide;
1238. 4-tert-Butyl-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
1239. 4-tert-Butyl-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
1240. 4-tert-Butyl-N-{1-[4-(2-hydroxy-pentanoyl)-6-oxo-hexahydro-pyrrolo[3,2-b]pyrrole-1-carbonyl]-3-methyl-butyl}-benzamide;
1241. 4-tert-Butyl-N-{1-[4-(2-methoxy-pentanoyl)-6-oxo-hexahydro-pyrrolo[3,2-b]pyrrole-1-carbonyl]-3-methyl-butyl}-benzamide;
1242. 4-tert-Butyl-N-{1-[4-(2-hydroxy-5-methyl-hexanoyl)-6-oxo-hexahydro-pyrrolo[3,2-b]pyrrole-1-carbonyl]-3-methyl-butyl}-benzamide;
1243. 4-tert-Butyl-N-{1-[4-(2-methoxy-5-methyl-hexanoyl)-6-oxo-hexahydro-pyrrolo[3,2-b]pyrrole-1-carbonyl]-3-methyl-butyl}-benzamide;
1244. N-[1-(4-Benzoyl-6-oxo-hexahydro-pyrrolo[3,2-b]pyrrole-1-carbonyl)-3-methyl-butyl]-4-dimethylaminobenzamide;
1245. 4-Dimethylamino-N-{3-methyl-1-[6-oxo-4-(1-oxy-pyridine-2-carbonyl)-hexahydro-pyrrolo[3,2-b]pyrrole-1-carbonyl]-butyl}-benzamide;
1246. 4-Dimethylamino-N-{3-methyl-1-[6-oxo-4-(pyridine-3-carbonyl)-hexahydro-pyrrolo[3,2-b]pyrrole-1-carbonyl]-butyl}-benzamide;
1247. 4-Dimethylamino-N-{3-methyl-1-[6-oxo-4-(1-oxy-pyridine-3-carbonyl)-hexahydro-pyrrolo[3,2-b]pyrrole-1-carbonyl]-butyl}-benzamide;
1248. 4-Dimethylamino-N-{3-methyl-1-[6-oxo-4-(pyridine-4-carbonyl)-hexahydro-pyrrolo[3,2-b]pyrrole-1-carbonyl]-butyl}-benzamide;
1249. 4-Dimethylamino-N-{3-methyl-1-[6-oxo-4-(1-oxy-pyridinecarbonyl)-hexahydro-pyrrolo[3,2-b]pyrrole-1-carbonyl]-butyl}-benzamide;
1250. N-[1-(4-Benzenesulfonyl-6-oxo-hexahydro-pyrrolo[3,2-b]pyrrole-1-carbonyl)-3-methyl-butyl]-4-dimethylamino-benzamide;
1251. 4-Dimethylamino-N-{3-methyl-1-[6-oxo-4-(pyridinesulfonyl)-hexahydro-pyrrolo[3,2-b]pyrrole-1-carbonyl]-butyl}-benzamide;
1252. 4-Dimethylamino-N-{3-methyl-1-[6-oxo-4-(1-oxy-pyridine-4-sulfonyl)-hexahydro-pyrrolo[3,2-b]pyrrole-1-carbonyl]-butyl}-benzamide;
1253. 4-Dimethylamino-N-[3-methyl-1-(6-oxo-4-phenylacetyl-hexahydro-pyrrolo[3,2-b]pyrrole-1-carbonyl)-butyl]-benzamide;
1254. 4-[2-(4-Dimethylamino-benzoylamino)methyl-pentanoyl]-3-oxo-hexahydro-pyrrolo[3,2-b]pyrrole-1-carboxylic acid phenylamide;
1255. 4-[2-(4-Dimethylamino-benzoylamino)-4-methyl-pentanoyl]-3-oxo-hexahydro-pyrrolo[3,2-b]pyrrole-1-carboxylic acid isobutyl-amide;
1256. 4-[2-(4-Dimethylamino-benzoylamino)-4-methyl-pentanoyl]-3-oxo-hexahydro-pyrrolo[3,2-b]pyrrole-1-carboxylic acid phenyl ester;
1257. 4-[2-(4-Dimethylamino-benzoylamino)-4-methyl-pentanoyl]-3-oxo-hexahydro-pyrrolo[3,2-b]pyrrole-1-carboxylic acid isobutyl ester,
1258. 4-Dimethylamino-N-{3-methyl-1-[6-oxo-4-(pyrrolidine-1-carbonyl)-hexahydro-pyrrolo[3,2-b]pyrrole-1-carbonyl]-butyl}-benzamide;
1259. 4-Dimethylamino-N-{3-methyl-1-[6-oxo-4-(piperidine-1-carbonyl)-hexahydro-pyrrolo[3,2-b]pyrrole-1-carbonyl]-butyl}-benzamide;
1260. 4-Dimethylamino-N-{3-methyl-1-[4-(morpholin-4-carbonyl)-6-oxo-hexahydro-pyrrolo[3,2-b]pyrrole-1-carbonyl]-butyl}-benzamide;
1261. 4-Dimethylamino-N-{3-methyl-1-[6-oxo-4-(piperazine-1-carbonyl)-hexahydro-pyrrolo[3,2-b]pyrrole-1-carbonyl]-butyl}-benzamide;
1262. 4-Dimethylamino-N-{3-methyl-1-[4-(4-methyl-piperazine-1-carbonyl)-6-oxo-hexahydro-pyrrolo[3,2-b]pyrrole-1-carbonyl]-butyl}-benzamide;
1263. 4-Dimethylamino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
1264. 4-Dimethylamino-N-{3-methyl-1-[6-oxo-4-(2-piperazin-1-yl-acetyl) hexahydro-pyrrolo[3,2-b]pyrrole-1-carbonyl]-butyl}-benzamide;
1265. 4-Dimethylamino-N-{1-[4-(furan-2-carbonyl)-6-oxo-hexahydro-pyrrolo[3,2-b]pyrrole-1-carbonyl]-3-methyl-butyl}-benzamide;
1266. 4-Dimethylamino-N-{1-[4-(5-chloro-furan-2-carbonyl)-6-oxo-hexahydro-pyrrolo[3,2-b]pyrrole-1-carbonyl]-3-methyl-butyl}-benzamide;
1267. 4-Dimethylamino-N-{1-[4-(thiophene-2-carbonyl)-6-oxo-hexahydro-pyrrolo[3,2-b]pyrrole-1-carbonyl]-3-methyl-butyl}-benzamide;
1268. 4-Dimethylamino-N-{1-[4-(5-chloro-thiophene-2-carbonyl)-6-oxo-hexahydro-pyrrolo[3,2-b]pyrrole-1-carbonyl]-3-methyl-butyl}-benzamide;
1269. 4-Dimethylamino-N-{1-[4-(furan-3-carbonyl)-6-oxo-hexahydro-pyrrolo[3,2-b]pyrrole-1-carbonyl]-3-methyl-butyl}-benzamide;
1270. 4-Dimethylamino-N-{1-[4-(thiophene-3-carbonyl)-6-oxo-hexahydro-pyrrolo[3,2-b]pyrrole-1-carbonyl]-3-methyl-butyl}-benzamide;
1271. 4-Dimethylamino-N-[1-(4-cyclopentanecarbonyl-6-oxo-hexahydro-pyrrolo[3,2-b]pyrrole-1-carbonyl)-3-methyl-butyl]-benzamide;
1272. 4-Dimethylamino-N-[1-(4-cyclohexanecarbonyl-6-oxo-hexahydro-pyrrolo[3,2-b]pyrrole-1-carbonyl)-3-methyl-butyl]-benzamide;
1273. 4-Dimethylamino-N-{1-[4-(2-cyclopent-3-enecarbonyl)-6-oxo-hexahydro-pyrrolo[3,2-b]pyrrole-1-carbonyl]-3-methyl-butyl}-benzamide;
1274. 4-Dimethylamino-N-{1-[4-(2-cyclopentyl-acetyl)-6-oxo-hexahydro-pyrrolo[3,2-b]pyrrole-1-carbonyl]-3-methyl-butyl}-benzamide;
1275. 4-Dimethylamino-N-{1-[4-(2-cyclohexyl-acetyl)-6-oxo-hexahydro-pyrrolo[3,2-b]pyrrole-1-carbonyl]-3-methyl-butyl}-benzamide;
1276. 4-Dimethylamino-N-{3-methyl-1-[6-oxo-4-(pyrrolidine-2-carbonyl)-hexahydro-pyrrolo[3,2-b]pyrrole-1-carbonyl]-butyl}-benzamide;
1277. N-{1-[4-(1-Acetyl-pyrrolidine-2-carbonyl)-6-oxo-hexahydro-pyrrolo[3,2-b]pyrrole-1-carbonyl]-3-methyl-butyl}-4-dimethylamino-benzamide;
1278. 4-Dimethylamino-N-{3-methyl-1-[6-oxo-4-(piperidine-2-carbonyl)-hexahydro-pyrrolo[3,2-b]pyrrole-1-carbonyl]-butyl}-benzamide;
1279. N-{1-[4-(1-Acetyl-piperidine-2-carbonyl)-6-oxo-hexahydro-pyrrolo[3,2-b]pyrrole-1-carbonyl]-3-methyl-butyl}-4-dimethylamino-benzamide;
1280. 4-Dimethylamino-N-{3-methyl-1-[6-oxo-4-(tetrahydro-furan-2-carbonyl)-hexahydro-pyrrolo[3,2-b]pyrrole-1-carbonyl]-butyl}-benzamide;
1281. N-{1-[4-(1-Amino-cyclobutanecarbonyl)-6-oxo-hexahydro-pyrrolo[3,2-b]pyrrole-1-carbonyl]-3-methyl-butyl}-4-dimethylamino-benzamide;
1282. N-{1-[4-(1-Amino-cyclopentanecarbonyl)-6-oxo-hexahydro-pyrrolo[3,2-b]pyrrole-1-carbonyl]-3-methyl-butyl}-4-dimethylamino-benzamide;
1283. N-{1-[4-(1-Amino-cyclohexanecarbonyl)-6-oxo-hexahydro-pyrrolo[3,2-b]pyrrole-1-carbonyl]-3-methyl-butyl}-4-dimethylamino-benzamide;
1284. N-{1-[4-(1-Acetylamino-cyclobutanecarbonyl)-6-oxo-hexahydro-pyrrolo[3,2-b]pyrrole-1-carbonyl]-3-methyl-butyl}-4-dimethylamino-benzamide;
1285. N-{1-[4-(1-Acetylamino-cyclopentanecarbonyl)-6-oxo-hexahydro-pyrrolo[3,2-b]pyrrole-1-carbonyl]-3-methyl-butyl}-4-dimethylamino-benzamide;
1286. N-{1-[4-(1-Acetylamino-cyclohexanecarbonyl)-6-oxo-hexahydro-pyrrolo[3,2-b]pyrrole-1-carbonyl]-3-methyl-butyl}-4-dimethylamino-benzamide;
1287. 4-Dimethylamino-N-{1-[4-(1-hydroxy-cyclobutanecarbonyl)-6-oxo-hexahydro-pyrrolo[3,2-b]pyrrole-1-carbonyl]-3-methyl-butyl}-benzamide;
1288. 4-Dimethylamino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
1289. 4-Dimethylamino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
1290. 4-Dimethylamino-N-{1-[4-methoxy-cyclobutanecarbonyl)-6-oxo-hexahydro-pyrrolo[3,2-b]pyrrole-1-carbonyl]-3-methyl-butyl}-benzamide;
1291. 4-Dimethylamino-N-{1-[4-(1-methoxy-cyclopentanecarbonyl)-6-oxo-hexahydro-pyrrolo[3,2-b]pyrrole-1-carbonyl]-3-methyl-butyl}-benzamide;
1292. 4-Dimethylamino-N-{1-[4-(1-methoxy-cyclohexanecarbonyl)-6-oxo-hexahydro-pyrrolo[3,2-b]pyrrole-1-carbonyl]-3-methyl-butyl}-benzamide;
1293. N-{1-[4-(2-Amino-2-cyclopentyl-acetyl)-6-oxo-hexahydro-pyrrolo[3,2-b]pyrrole-1-carbonyl]-3-methyl-butyl}-4-dimethylamino-benzamide;
1294. N-{1-[4-(2-Amino-2-cyclohexyl-acetyl)-6-oxo-hexahydro-pyrrolo[3,2-b]pyrrole-1-carbonyl]-3-methyl-butyl}-4-dimethylamino-benzamide;
1295. N {1-[4-(2-Acetylamino-2-cyclopentyl-acetyl)-6-oxo-hexahydro-pyrrolo[3,2-b]pyrrole-1-carbonyl]-3-methyl-butyl}-4-dimethylamino-benzamide;
1296. N-{1-[4-(2-Acetylamino-2-cyclohexyl-acetyl)-6-oxo-hexahydro-pyrrolo[3,2-b]pyrrole-1-carbonyl]-3-methyl-butyl}-4-dimethylamino-benzamide;
1297. N-{1-[4-(2-Acetylamino-propionyl)-6-oxo-hexahydro-pyrrolo[3,2-b]pyrrole-1-carbonyl]-3-methyl-butyl}-4-dimethylamino-benzamide;
1298. N-{1-[4-(2-Acetylamino-acetyl)-6-oxo-hexahydro-pyrrolo[3,2-b]pyrrole-1-carbonyl]-3-methyl-butyl}-4-dimethylamino-benzamide;
1299. N-(1-{4-[2-(Acetyl-methyl-amino)-acetyl]-6-oxo-hexahydro-pyrrolo[3,2-b]pyrrole-1-carbonyl}-3-methyl-butyl)-4-dimethylamino-benzamide;
1300. 4-Dimethylamino-N-{1-[4-(2-dimethylamino-acetyl)-6-oxo-hexahydro-pyrrolo[3,2-b]pyrrole-1-carbonyl]-3-methyl-butyl}-benzamide;
1301. N-{1-[4-(2-Amino-butyryl)-6-oxo-hexahydro-pyrrolo[3,2-b]pyrrole-1-carbonyl]-3-methyl-butyl}-4-dimethylamino-benzamide;
1302. N-{1-[4-(2-Acetylamino-butyryl)-6-oxo-hexahydro-pyrrolo[3,2-b]pyrrole-1-carbonyl]-3-methyl-butyl}-4-dimethylamino-benzamide;
1303. N-{1-[4-(2-amino-3-methyl-butyryl)-6-oxo-hexahydro-pyrrolo[3,2-b]pyrrole-1-carbonyl]-3-methyl-butyl}-4-dimethylamino-benzamide;
1304. N-{1-[4-(2-Acetylamino-3-methyl-butyryl)-6-oxo-hexahydro-pyrrolo[3,2-b]pyrrole-1-carbonyl]-3-methyl-butyl}-4-dimethylamino-benzamide;
1305. N-{1-[4-(2-amino-3,3-dimethyl-butyryl)-6-oxo-hexahydro-pyrrolo[3,2-b]pyrrole-1-carbonyl]-3-methyl-butyl}-4-dimethylamino-benzamide;
1306. N-{1-[4-(2-Acetylamino-3,3-dimethyl-butyryl)-6-oxo-hexahydro-pyrrolo[3,2-b]pyrrole-1-carbonyl]-3-methyl-butyl}-4-dimethylamino-benzamide;
1307. N-{1-[4-(2-Amino-3-methyl-pentanoyl)-6-oxo-hexahydro-pyrrolo[3,2-b]pyrrole-1-carbonyl]-3-methyl-butyl}-4-dimethylamino-benzamide;
1308. N-{1-[4-(2-Acetylamino-3-methyl-pentanoyl)-6-oxo-hexahydro-pyrrolo[3,2-b]pyrrole-1-carbonyl]-3-methyl-butyl}-4-dimethylamino-benzamide;
1309. 4-Dimethylamino-N-[3-methyl-1-(6-oxo-4-pentanoyl-hexahydro-pyrrolo[3,2-b]pyrrole-1-carbonyl)-butyl]-benzamide;
1310. 4-Dimethylamino-N-{3-methyl-1-[4-(3-methyl-pentanoyl)-6-oxo-hexahydro-pyrrolo[3,2-b]pyrrole-1-carbonyl]-butyl}-benzamide;
1311. 4-Dimethylamino-N-{3-methyl-1-[4-(4-methyl-pentanoyl)-6-oxo-hexahydro-pyrrolo[3,2-b]pyrrole-1-carbonyl]-butyl}-benzamide;
1312. N-{1-[4-(2-Amino-4-methyl-pentanoyl)-6-oxo-hexahydro-pyrrolo[3,2-b]pyrrole-1-carbonyl]-3-methyl-butyl}-4-dimethylamino-benzamide;
1313. N-{1-[4-(2-Acetylamino-4-methyl-pentanoyl)-6-oxo-hexahydro-pyrrolo[3,2-b]pyrrole-1-carbonyl]-3-methyl-butyl}-4-dimethyl amino-benzamide;
1314. N-{1-[4-(2-Amino-4,4-dimethyl-pentanoyl)-6-oxo-hexahydro-pyrrolo[3,2-b]pyrrole-1-carbonyl]-3-methyl-butyl}-4-dimethylamino-benzamide;
1315. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-dimethylamino-benzamide;
1316. N-{1-[4-(2-Amino-pentanoyl)-6-oxo-hexahydro-pyrrolo[3,2-b]pyrrole-1-carbonyl]-3-methyl-butyl}-4-dimethylamino-benzamide;
1317. N-{1-[4-(2-Acetylamino-pentanoyl)-6-oxo-hexahydro-pyrrolo[3,2-b]pyrrole-1-carbonyl]-3-methyl-butyl}-4-dimethylamino-benzamide;
1318. N-{1-[4-(2-Amino-5-methyl-hexanoyl)-6-oxo-hexahydro-pyrrolo[3,2-b]pyrrole-1-carbonyl]-3-methyl-butyl}-4-dimethylamino-benzamide;
1319. N-{1-[4-(2-Acetylamino-5-methyl-hexanoyl)-6-oxo-hexahydro-pyrrolo[3,2-b]pyrrole-1-carbonyl]-3-methyl-butyl}-4-dimethylamino-benzamide;
1320. N-{1-[4-(2-hydroxy-3-methyl-butyryl)-6-oxo-hexahydro-pyrrolo[3,2-b]pyrrole-1-carbonyl]-3-methyl-butyl}-4-dimethylamino-benzamide;
1321. N-{1-[4-(2-methoxy-3-methyl-butyryl)-6-oxo-hexahydro-pyrrolo[3,2-b]pyrrole-1-carbonyl]-3-methyl-butyl}-4-dimethylamino-benzamide;
1322. 4-Dimethylamino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
1323. 4-Dimethylamino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
1324. 4-Dimethylamino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
1325. 4-Dimethylamino-N-{1-[4-(2-methoxy-3-methyl-pentanoyl)-6-oxo-hexahydro-pyrrolo[3,2-b]pyrrole-1-carbonyl]-3-methyl-butyl}-benzamide;
1326. 4-Dimethylamino-N-{1-[4-(2-hydroxy-4-methyl-pentanoyl)-6-oxo-hexahydro-pyrrolo[3,2-b]pyrrole-1-carbonyl]-3-methyl-butyl}-benzamide;
1327. 4-Dimethylamino-N-{1-[4-(2-methoxy-4-methyl-pentanoyl)-6-oxo-hexahydro-pyrrolo[3,2-b]pyrrole-1-carbonyl]-3-methyl-butyl}-benzamide;
1328. 4-Dimethylamino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
1329. 4-Dimethylamino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
1330. 4-Dimethylamino-N-{1-[4-(2-hydroxy-pentanoyl)-6-oxo-hexahydro-pyrrolo[3,2-b]pyrrole-1-carbonyl]-3-methyl-butyl}-benzamide;
1331. 4-Dimethylamino-N-{1-[4-(2-methoxy-pentanoyl)-6-oxo-hexahydro-pyrrolo[3,2-b]pyrrole-1-carbonyl]-3-methyl-butyl}-benzamide;
1332. 4-Dimethylamino-N-{1-[4-(2-hydroxy-5-methyl-hexanoyl)-6-oxo-hexahydro-pyrrolo[3,2-b]pyrrole-1-carbonyl]-3-methyl-butyl}-benzamide;
1333. 4-Dimethylamino-N-{1-[4-(2-methoxy-5-methyl-hexanoyl)-6-oxo-hexahydro-pyrrolo[3,2-b]pyrrole-1-carbonyl]-3-methyl-butyl}-benzamide;
1334. N-[1-(4-Benzoyl-6-oxo-hexahydro-pyrrolo[3,2-b]pyrrole-1-carbonyl)-3-methyl-butyl]-4-thiophen-2-yl-benzamide;
1335. N-{3-methyl-1-[6-oxo-4-(1-oxy-pyridine-2-carbonyl)-hexahydro-pyrrolo[3,2-b]pyrrole-1-carbonyl]-butyl}-4-thiophen-2-yl-benzamide;
1336. N-{3-methyl-1-[6-oxo-4-(pyridine-3-carbonyl)-hexahydro-pyrrolo[3,2-b]pyrrole-1-carbonyl]-butyl}-4-thiophen-2-yl-benzamide;
1337. N-{3-methyl-1-[6-oxo-4-(1-oxy-pyridine-3-carbonyl)-hexahydro-pyrrolo[3,2-b]pyrrole-1-carbonyl]-butyl}-4-thiophen-2-yl-benzamide;
1338. N-{3-methyl-1-[6-oxo-4-(pyridine-4-carbonyl)-hexahydro-pyrrolo[3,2-b]pyrrole-1-carbonyl]-butyl}-4-thiophen-2-yl-benzamide;
1339. N-{3-methyl-1-[6-oxo-4-(1-oxy-pyridine-4-carbonyl)-hexahydro-pyrrolo[3,2-b]pyrrole-1-carbonyl]-butyl}-4-thiophen-2-yl-benzamide;
1340. N-[1-(4-Benzenesulfonyl-6-oxo-hexahydro-pyrrolo[3,2-b]pyrrole-1-carbonyl)-3-methyl-butyl]-4-thiophen-2-yl-benzamide;
1341. N-{3-methyl-1-[6-oxo-4-(pyridinesulfonyl)hexahydro-pyrrolo[3,2-b]pyrrole-1-carbonyl]-butyl}-4-thiophen-2-yl-benzamide;
1342. N-{3-methyl-1-[6-oxo-4-(1-oxy-pyridinesulfonyl)hexahydro-pyrrolo[3,2-b]pyrrole-1-carbonyl]-butyl}-4-thiophen-2-yl-benzamide;
1343. N-[3-methyl-1-(6-oxo-4-phenylacetyl-hexahydro-pyrrolo[3,2-b]pyrrole-1-carbonyl)-butyl]-4-thiophen-2-yl-benzamide;
1344. 4-[4-Methyl-2-(4-thiophen-2-yl-benzoylamino)-pentanoyl]-3-oxo-hexahydro-pyrrolo[3,2-b]pyrrole-1-carboxylic acid phenylamide;
1345. 4-[4-Methyl-2-(4-thiophen-2-yl-benzoylamino)-pentanoyl]-3-oxo-hexahydro-pyrrolo[3,2-b]pyrrole-1-carboxylic acid isobutyl-amide;
1346. 4-[4-Methyl-2-(4-thiophen-2-yl-benzoylamino)-pentanoyl]-3-oxo-hexahydro-pyrrolo[3,2-b]pyrrole-1-carboxylic acid phenyl ester,
1347. 4-[4-Methyl-2-(4-thiophen-2-yl-benzoylamino)-pentanoyl]-3-oxo-hexahydro-pyrrolo[3,2-b]pyrrole-1-carboxylic acid isobutyl ester,
1348. N-{3-methyl-1-[6-oxo-4-(pyrrolidine-1-carbonyl)-hexahydro-pyrrolo[3,2-b]pyrrole-1-carbonyl]-butyl}-4-thiophen-2-yl-benzamide;
1349. N-{3-methyl-1-[6-oxo-4-(piperidine-1-carbonyl)-hexahydro-pyrrolo[3,2-b]pyrrole-1-carbonyl]-butyl}-4-thiophen-2-yl-benzamide;
1350. N-{3-methyl-1-[4-(morpholin-4-carbonyl)-6-oxo-hexahydro-pyrrolo[3,2-b]pyrrole-1-carbonyl]-butyl}-4-thiophen-2-yl-benzamide;
1351. N-{3-methyl-1-[6-oxo-4-(piperazine-1-carbonyl)-hexahydro-pyrrolo[3,2-b]pyrrole-1-carbonyl]-butyl}-4-thiophen-2-yl-benzamide;
1352. N-{3-methyl-1-[4-(4-methyl-piperazine-1-carbonyl)-6-oxo-hexahydro-pyrrolo[3,2-b]pyrrole-1-carbonyl]-butyl}-4-thiophen-2-yl-benzamide;
1353. N-(3-methyl-1-{4-[2-(4-methyl-piperazine-1-yl)-acetyl]-6-oxo-hexahydro-pyrrolo[3,2-b]pyrrole-1-carbonyl}-butyl)-4-thiophen-2-yl-benzamide;
1354. N-{3-methyl-1-[6-oxo-4-(2-piperazin-1-yl-acetyl)-hexahydro-pyrrolo[3,2-b]pyrrole-1-carbonyl]-butyl}-4-thiophen-2-yl-benzamide;
1355. N-{1-[4-(furan-2-carbonyl)-6-oxo-hexahydro-pyrrolo[3,2-b]pyrrole-1-carbonyl]-3-methyl-butyl}-4-thiophen-2-yl-benzamide;
1356. N-{1-[4-(5-chloro-furan-2-carbonyl)-6-oxo-hexahydro-pyrrolo[3,2-b]pyrrole-1-carbonyl]-3-methyl-butyl}-4-thiophen-2-yl-benzamide;
1357. N-{1-[4-(thiophene-2-carbonyl)-6-oxo-hexahydro-pyrrolo[3,2-b]pyrrole-1-carbonyl]-3-methyl-butyl}-4-thiophen-2-yl-benzamide;
1358. N-{1-[4-(5-chloro-thiophene-2-carbonyl)-6-oxo-hexahydro-pyrrolo[3,2-b]pyrrole-1-carbonyl]-3-methyl-butyl}-4-thiophen-2-yl-benzamide;
1359. N-{1-[4-(furan-3-carbonyl)-6-oxo-hexahydro-pyrrolo[3,2-b]pyrrole-1-carbonyl]-3-methyl-butyl}-4-thiophen-2-yl-benzamide;
1360. N-{1-[4-(thiophene-3-carbonyl)-6-oxo-hexahydro-pyrrolo[3,2-b]pyrrole-1-carbonyl]-3-methyl-butyl}-4-thiophen-2-yl-benzamide;
1361. N-[1-(4-cyclopentanecarbonyl-6-oxo-hexahydro-pyrrolo[3,2-b]pyrrole-1-carbonyl)-3-methyl-butyl]-4-thiophen-2-yl-benzamide;
1362. N-[1-(4-cyclohexanecarbonyl-6-oxo-hexahydro-pyrrolo[3,2-b]pyrrole-1-carbonyl)-3-methyl-butyl]-4-thiophen-2-yl-benzamide;
1363. N-{1-[4-(cyclopent-3-enecarbonyl)-6-oxo-hexahydro-pyrrolo[3,2-b]pyrrole-1-carbonyl]-3-methyl-butyl}-4-thiophen-2-yl-benzamide;
1364. N-{1-[4-(2-cyclopentyl-acetyl)-6-oxo-hexahydro-pyrrolo[3,2-b]pyrrole-1-carbonyl]-3-methyl-butyl}-4-thiophen-2-yl-benzamide;
1365. N-{1-[4-(2-cyclohexyl-acetyl)-6-oxo-hexahydro-pyrrolo[3,2-b]pyrrole-1-carbonyl]-3-methyl-butyl}-4-thiophen-2-yl-benzamide;
1366. N-{3-methyl-1-[6-oxo-4-(pyrrolidine-2-carbonyl)-hexahydro-pyrrolo[3,2-b]pyrrole-1-carbonyl]-butyl}-4-thiophen-2-yl-benzamide;
1367. N-{1-[4-(1-Acetyl-pyrrolidine-2-carbonyl)-6-oxo-hexahydro-pyrrolo[3,2-b]pyrrole-1-carbonyl]-3-methyl-butyl}-4-thiophen-2-yl-benzamide;
1368. N-{3-methyl-1-[6-oxo-4-(piperidine-2-carbonyl)-hexahydro-pyrrolo[3,2-b]pyrrole-1-carbonyl]-butyl}-4-thiophen-2-yl-benzamide;
1369. N-{1-[4-(1-Acetyl-piperidine-2-carbonyl)-6-oxo-hexahydro-pyrrolo[3,2-b]pyrrole-1-carbonyl]-3-methyl-butyl}-4-thiophen-2-yl-benzamide;
1370. N-{3-methyl-1-[6-oxo-4-(tetrahydro-furan-2-carbonyl)-hexahydro-pyrrolo[3,2-b]pyrrole-1-carbonyl]-butyl}-4-thiophen-2-yl-benzamide;
1371. N-{1-[4-(1-Amino-cyclobutanecarbonyl)-6-oxo-hexahydro-pyrrolo[3,2-b]pyrrole-1-carbonyl]-3-methyl-butyl}-4-thiophen-2-yl-benzamide;
1372. N-{1-[4-(1-Amino-cyclopentanecarbonyl)-6-oxo-hexahydro-pyrrolo[3,2-b]pyrrole-1-carbonyl]-3-methyl-butyl}-4-thiophen-2-yl-benzamide;
1373. N-{1-[4-(1-Amino-cyclohexanecarbonyl)-6-oxo-hexahydro-pyrrolo[3,2-b]pyrrole-1-carbonyl]-3-methyl-butyl}-4-thiophen-2-yl-benzamide;
1374. N-{1-[4-(1-Acetylamino-cyclobutanecarbonyl)-6-oxo-hexahydro-pyrrolo[3,2-b]pyrrole-1-carbonyl]-3-methyl-butyl}-4-thiophen-2-yl-benzamide;
1375. N{1-[4-(1-Acetylamino-cyclopentanecarbonyl)-6-oxo-hexahydro-pyrrolo[3,2-b]pyrrole-1-carbonyl]-3-methyl-butyl}-4-thiophen-2-yl-benzamide;
1376. N-{1-[4-(1-Acetylamino-cyclohexanecarbonyl)-6-oxo-hexahydro-pyrrolo[3,2-b]pyrrole-1-carbonyl]-3-methyl-butyl}-4-thiophen-2-yl-benzamide;
1377. N-{1-[4-(1-hydroxy-cyclobutanecarbonyl)-6-oxo-hexahydro-pyrrolo[3,2-b]pyrrole-1-carbonyl]-3-methyl-butyl}-4-thiophen-2-yl-benzamide;
1378. N-{1-[4-(1-hydroxy-cyclopentanecarbonyl)-6-oxo-hexahydro-pyrrolo[3,2-b]pyrrole-1-carbonyl]-3-methyl-butyl}-4-thiophen-2-yl-benzamide;
1379. N-{1-[4-(1-hydroxy-cyclohexanecarbonyl)-6-oxo-hexahydro-pyrrolo[3,2-b]pyrrole-1-carbonyl]-3-methyl-butyl}-4-thiophen-2-yl-benzamide;
1380. N-{1-[4-(1-methoxy-cyclobutanecarbonyl)-6-oxo-hexahydro-pyrrolo[3,2-b]pyrrole-1-carbonyl]-3-methyl-butyl}-4-thiophen-2-yl-benzamide;
1381. N-{1-[4-(1-methoxy-cyclopentanecarbonyl)-6-oxo-hexahydro-pyrrolo[3,2-b]pyrrole-1-carbonyl]-3-methyl-butyl}-4-thiophen-2-yl-benzamide;
1382. N-{1-[4-(1-methoxy-cyclohexanecarbonyl)-6-oxo-hexahydro-pyrrolo[3,2-b]pyrrole-1-carbonyl]-3-methyl-butyl}-4-thiophen-2-yl-benzamide;
1383. N-{1-[4-(2-Amino-2-cyclopentyl-acetyl)-6-oxo-hexahydro-pyrrolo[3,2-b]pyrrole-1-carbonyl]-3-methyl-butyl}-4-thiophen-2-yl-benzamide;
1384. N-{1-[4-(2-Amino-2-cyclohexyl-acetyl)-6-oxo-hexahydro-pyrrolo[3,2-b]pyrrole-1-carbonyl]-3-methyl-butyl}-4-thiophen-2-yl-benzamide;
1385. N-{1-[4-(2-Acetylamino-2-cyclopentyl-acetyl)-6-oxo-hexahydro-pyrrolo[3,2-b]pyrrole-1-carbonyl]-3-methyl-butyl}-4-thiophen-2-yl-benzamide;
1386. N-{1-[4-(2-Acetylamino-2-cyclohexyl-acetyl)-6-oxo-hexahydro-pyrrolo[3,2-b]pyrrole-1-carbonyl]-3-methyl-butyl}-4-thiophen-2-yl-benzamide;
1387. N-{1-[4-(2-Acetylamino-propionyl)-6-oxo-hexahydro-pyrrolo[3,2-b]pyrrole-1-carbonyl]-3-methyl-butyl}-4-thiophen-2-yl-benzamide;
1388. N-{1-[4-(2-Acetylamino-acetyl)-6-oxo-hexahydro-pyrrolo[3,2-b]pyrrole-1-carbonyl]-3-methyl-butyl}-4-thiophen-2-yl-benzamide;
1389. N-(1-{4-[2-(Acetyl-methyl-amino)-acetyl]-6-oxo-hexahydro-pyrrolo[3,2-b]pyrrole-1-carbonyl}-3-methyl-butyl)-4-thiophen-2-yl-benzamide;
1390. N-{1-[4-(2-dimethylamino-acetyl)-6-oxo-hexahydro-pyrrolo[3,2-b]pyrrole-1-carbonyl]-3-methyl-butyl}-4-thiophen-2-yl-benzamide;
1391. N-{1-[4-(2-Amino-butyryl)-6-oxo-hexahydro-pyrrolo[3,2-b]pyrrole-1-carbonyl]-3-methyl-butyl}-4-thiophen-2-yl-benzamide;
1392. N-{1-[4-(2-Acetylamino-butyryl)-6-oxo-hexahydro-pyrrolo[3,2-b]pyrrole-1-carbonyl]-3-methyl-butyl}-4-thiophen-2-yl-benzamide;
1393. N-{1-[4-(2-amino-3-methyl-butyryl)-6-oxo-hexahydro-pyrrolo[3,2-b]pyrrole-1-carbonyl]-3-methyl-butyl}-4-thiophen-2-yl-benzamide;
1394. N-{1-[4-(2-Acetylamino-3-methyl-butyryl)-6-oxo-hexahydro-pyrrolo[3,2-b]pyrrole-1-carbonyl]-3-methyl-butyl}-6-thiophen-2-yl-benzamide;
1395. N-{1-[4-(2-amino-3,3-dimethyl-butyryl)-6-oxo-hexahydro-pyrrolo[3,2-b]pyrrole-1-carbonyl]-3-methyl-butyl}-4-thiophen-2-yl-benzamide;
1396. N-{1-[4-(2-Acetylamino-3,3-dimethyl-butyryl)-6-oxo-hexahydro-pyrrolo[3,2-b]pyrrole-1-carbonyl]-3-methyl-butyl}-4-thiophen-2-yl-benzamide;
1397. N-{1-[4-(2-Amino-3-methyl-pentanoyl)-6-oxo-hexahydro-pyrrolo[3,2-b]pyrrole-1-carbonyl]-3-methyl-butyl}-4-thiophen-2-yl-benzamide;
1398. N-{1-[4-(2-Acetylamino-3-methyl-pentanoyl)-6-oxo-hexahydro pyrrolo[3,2-b]pyrrole-1-carbonyl]-3-methyl-butyl}-4-thiophen-2-yl-benzamide;
1399. N-[3-methyl-1-(6-oxo-4-pentanoyl-hexahydro-pyrrolo[3,2-b]pyrrole-1-carbonyl)-butyl]-4-thiophen-2-yl-benzamide;
1400. N-{3-methyl-1-[4-(3-methyl-pentanoyl)-6-oxo-hexahydro-pyrrolo[3,2-b]pyrrole-1-carbonyl]-butyl}-4-thiophen-2-yl-benzamide;
1401. N-{3-methyl-1-[4-(4-methyl-pentanoyl)-6-oxo-hexahydro-pyrrolo[3,2-b]pyrrole-1-carbonyl]-butyl}-4-thiophen-2-yl-benzamide;
1402. N-{1-[4-(2-Amino-4-methyl-pentanoyl)-6-oxo-hexahydro-pyrrolo[3,2-b]pyrrole-1-carbonyl]-3-methyl-butyl}-4-thiophen-2-yl-benzamide;
1403. N-{1-[4-(2-Acetylamino-4-methyl-pentanoyl)-6-oxo-hexahydro-pyrrolo[3,2-b]pyrrole-1-carbonyl]-3-methyl-butyl}-4-thiophen-2-yl-benzamide;
1404. N-{1-[4-(2-Amino-4,4-dimethyl-pentanoyl)-6-oxo-hexahydro-pyrrolo[3,2-b]pyrrole-1-carbonyl]-3-methyl-butyl}-4-thiophen-2-yl-benzamide;
1405. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-thiophen-2-yl-benzamide;
1406. N-{1-[4-(2-Amino-pentanoyl)-6-oxo-hexahydro-pyrrolo[3,2-b]pyrrole-1-carbonyl]-3-methyl-butyl}-4-thiophen-2-yl-benzamide;
1407. N-{1-[4-(2-Acetylamino-pentanoyl)-6-oxo-hexahydro-pyrrolo[3,2-b]pyrrole-1-carbonyl]-3-methyl-butyl}-4-thiophen-2-yl-benzamide;
1408. N-{1-[4-(2-Amino-5-methyl-hexanoyl)-6-oxo-hexahydro-pyrrolo[3,2-b]pyrrole-1-carbonyl]-3-methyl-butyl}-4-thiophen-2-yl-benzamide;
1409. N-{1-[4-(2-Acetylamino-5-methyl-hexanoyl)-6-oxo-hexahydro-pyrrolo[3,2-b]pyrrole-1-carbonyl]-3-methyl-butyl}-4-thiophen-2-yl-benzamide;
1410. N-{1-[4-(2-hydroxy-3-methyl-butyryl)-6-oxo-hexahydro-pyrrolo[3,2-b]pyrrole-1-carbonyl]-3-methyl-butyl}-4-thiophen-2-yl-benzamide;
1411. N-{1-[4-(2-methoxy-3-methyl-butyryl)-6-oxo-hexahydro-pyrrolo[3,2-b]pyrrole-1-carbonyl]-3-methyl-butyl}-4-thiophen-2-yl-benzamide;
1412. N-{1-[4-(2-hydroxy-3,3-dimethyl-butyryl)-6-oxo-hexahydro-pyrrolo[3,2-b]pyrrole-1-carbonyl]-3-methyl-butyl}-4-thiophen-2-yl-benzamide;
1413. N-{1-[4-(2-methoxy-3,3-dimethyl-butyryl)-6-oxo-hexahydro-pyrrolo[3,2-b]pyrrole-1-carbonyl]-3-methyl-butyl}-4-thiophen-2-yl-benzamide;
1414. N-{1-[4-(2-hydroxy-3-methyl-pentanoyl)-6-oxo-hexahydro-pyrrolo[3,2-b]pyrrole-1-carbonyl]-3-methyl-butyl}-4-thiophen-2-yl-benzamide,
1415. N-{1-[4-(2-methoxy-3-methyl-pentanoyl)-6-oxo-hexahydro-pyrrolo[3,2-b]pyrrole-1-carbonyl]-3-methyl-butyl}-4-thiophen-2-yl-benzamide;
1416. N-{1-[4-(2-hydroxy methyl-pentanoyl)-6-oxo-hexahydro-pyrrolo[3,2-b]pyrrole-1-carbonyl]-3-methyl-butyl}-4-thiophen-2-yl-benzamide;
1417. N-{1-[4-(2-methoxy-4-methyl-pentanoyl)-6-oxo-hexahydro-pyrrolo[3,2-b]pyrrole-1-carbonyl]-3-methyl-butyl}-4-thiophen-2-yl-benzamide;
1418. N-{1-[4-(2-hydroxy-4,4-dimethyl-pentanoyl)-6-oxo-hexahydro-pyrrolo[3,2-b]pyrrole-1-carbonyl]-3-methyl-butyl}-4-thiophen-2-yl-benzamide;
1419. N-{1-[4-(2-methoxy-4,4-dimethyl-pentanoyl)-6-oxo-hexahydro-pyrrolo[3,2-b]pyrrole-1-carbonyl]-3-methyl-butyl}-4-thiophen-2-yl-benzamide;
1420. N-{1-[4-(2-hydroxy-pentanoyl)-6-oxo-hexahydro-pyrrolo[3,2-b]pyrrole-1-carbonyl]-3-methyl-butyl}-4-thiophen-2-yl-benzamide;
1421. N-{1-[4-(2-methoxy-pentanoyl)-6-oxo-hexahydro-pyrrolo[3,2-b]pyrrole-1-carbonyl]-3-methyl-butyl}-4-thiophen-2-yl-benzamide;
1422. N-{1-[4-(2-hydroxy-5-methyl-hexanoyl)-6-oxo-hexahydro-pyrrolo[3,2-b]pyrrole-1-carbonyl]-3-methyl-butyl}-4-thiophen-2-yl-benzamide;
1423. N-{1-[4-(2-methoxy-5-methyl-hexanoyl)-6-oxo-hexahydro-pyrrolo[3,2-b]pyrrole-1-carbonyl]-3-methyl-butyl}-4-thiophen-2-yl-benzamide;
1424. 5-Phenyl-thiophene-2-carboxylic acid-[1-(4-Benzoyl-6-oxo-hexahydro-pyrrolo[3,2-b]pyrrole-1-carbonyl)-3-methyl-butyl]-amide;
1425. 5-Phenyl-thiophene-2-carboxylic acid-{3-methyl-1-[6-oxo-4-(pyridine-2-carbonyl)-hexahydro-pyrrolo[3,2-b]pyrrole-1-carbonyl]-butyl}-amide;
1426. 5-Phenyl-thiophene-2-carboxylic acid-{3-methyl-1-[6-oxo-4-(1-oxy-pyridine-2-carbonyl)-hexahydro-pyrrolo[3,2-b]pyrrole-1-carbonyl]-butyl}-amide;
1427. 5-Phenyl-thiophene-2-carboxylic acid-{3-methyl-1-[6-oxo-4-(pyridine-3-carbonyl)-hexahydro-pyrrolo[3,2-b]pyrrole-1-carbonyl]-butyl}-amide;
1428. 5-Phenyl-thiophene-2-carboxylic acid-{3-methyl-1-[6-oxo-4-(1-oxy-pyridine-3-carbonyl)-hexahydro-pyrrolo[3,2-b]pyrrole-1-carbonyl]-butyl}-amide;
1429. 5-Phenyl-thiophene-2-carboxylic acid-{3-methyl-1-[6-oxo-4-(pyridine-4-carbonyl)-hexahydro-pyrrolo[3,2-b]pyrrole-1-carbonyl]-butyl}-amide;
1430. 5-Phenyl-thiophene-2-carboxylic acid-{3-methyl-1-[6-oxo-4-(1-oxy-pyridine-4-carbonyl)-hexahydro-pyrrolo[3,2-b]pyrrole-1-carbonyl]-butyl}-amide;
1431. 5-Phenyl-thiophene-2-carboxylic acid-[1-(4-Benzenesulfonyl-6-oxo-hexahydro-pyrrolo[3,2-b]pyrrole-1-carbonyl)-3-methyl-butyl]-amide;
1432. 5-Phenyl-thiophene-2-carboxylic acid-{3-methyl-1-[6-oxo-4-(pyridine-2-sulfonyl)-hexahydro-pyrrolo[3,2-b]pyrrole-1-carbonyl]-butyl}-amide;
1433. 5-Phenyl-thiophene-2-carboxylic acid-{3-methyl-1-[6-oxo-4-(1-oxy-pyridine-2-sulfonyl)-hexahydro-pyrrolo[3,2-b]pyrrole-1-carbonyl]-butyl}-amide;
1434. 5-Phenyl-thiophene-2-carboxylic acid-{3-methyl-1-[6-oxo-4-(pyridine-3-sulfonyl)-hexahydro-pyrrolo[3,2-b]pyrrole-1-carbonyl]-butyl}-amide;
1435. 5-Phenyl-thiophene-2-carboxylic acid-{3-methyl-1-[6-oxo-4-(1-oxy-pyridine-3-sulfonyl)-hexahydro-pyrrolo[3,2-b]pyrrole-1-carbonyl]-butyl}-amide;
1436. 5-Phenyl-thiophene-2-carboxylic acid-{3-methyl-1-[6-oxo-4-(pyridine-4-sulfonyl)-hexahydro-pyrrolo[3,2-b]pyrrole-1-carbonyl]-butyl}-amide;
1437. 5-Phenyl-thiophene-2-carboxylic acid-{3-methyl-1-[6-oxo-4-(1-oxy-pyridine-4-sulfonyl)-hexahydro-pyrrolo[3,2-b]pyrrole-1-carbonyl]-butyl}-amide;
1438. 5-Phenyl-thiophene-2-carboxylic acid-[3-methyl-1-(6-oxo-4-phenylacetyl-hexahydro-pyrrolo[3,2-b]pyrrole-1-carbonyl)-butyl]-amide;
1439. 4-{4-Methyl-2-[(5-phenyl-thiophene-2-carbonyl)-amino]-pentanoyl}-3-oxo-hexahydro-pyrrolo[3,2-b]pyrrole-1-carboxylic acid phenylamide;
1440. 4-{4-Methyl-2-[(5-phenyl-thiophene-2-carbonyl)-amino]-pentanoyl}-3-oxo-hexahydro-pyrrolo[3,2-b]pyrrole-1-carboxylic acid isobutyl-amide;
1441. 4-{4-Methyl-2-[(5-phenyl-thiophene-2-carbonyl)-amino]-pentanoyl}-3-oxo-hexahydro-pyrrolo[3,2-b]pyrrole-1-carboxylic acid phenyl ester;
1442. 4-{4-Methyl-2-[(5-phenyl-thiophene-2-carbonyl)-amino]-pentanoyl}-3-oxo-hexahydro-pyrrolo[3,2-b]pyrrole-1-carboxylic acid isobutyl ester;
1443. 5-Phenyl-thiophene-2-carboxylic acid-{3-methyl-1-[6-oxo-4-(pyrrolidine-1-carbonyl)-hexahydro-pyrrolo[3,2-b]pyrrole-1-carbonyl]-butyl}-amide;
1444. 5-Phenyl-thiophene-2-carboxylic acid-{3-methyl-1-[6-oxo-4-(piperidine-1-carbonyl)-hexahydro-pyrrolo[3,2-b]pyrrole-1-carbonyl]-butyl}-amide;
1445. 5-Phenyl-thiophene-2-carboxylic acid-{3-methyl-1-[4-(morpholin-4-carbonyl)-6-oxo-hexahydro-pyrrolo[3,2-b]pyrrole-1-carbonyl]-butyl}-amide;
1446. 5-Phenyl-thiophene-2-carboxylic acid-{3-methyl-1-[6-oxo-4-(piperazine-1-carbonyl)-hexahydro-pyrrolo[3,2-b]pyrrole-1-carbonyl]-butyl}-amide;
1447. 5-Phenyl-thiophene-2-carboxylic acid-{3-methyl-1-[4-(4-methyl-piperazine-1-carbonyl)-6-oxo-hexahydro-pyrrolo[3,2-b]pyrrole-1-carbonyl]-butyl}-amide;
1448. 5-Phenyl-thiophene-2-carboxylic acid-(3-methyl-1-{4-[2-(4-methyl-piperazin-1-yl)-acetyl]-6-oxo-hexahydro-pyrrolo[3,2-b]pyrrole-1-carbonyl}-butyl)-amide;
1449. 5-Phenyl-thiophene-2-carboxylic acid-{3-methyl-1-[6-oxo-4-(2-piperazin-1-yl-acetyl)-hexahydro-pyrrolo[3,2-b]pyrrole-1-carbonyl]-butyl}-amide;
1450. 5-Phenyl-thiophene-2-carboxylic acid-{1-[4-(furan-2-carbonyl)-6-oxo-hexahydro-pyrrolo[3,2-b]pyrrole-1-carbonyl]-3-methyl-butyl}-amide;
1451. 5-Phenyl-thiophene-2-carboxylic acid-{1-[4-(5-chloro-furan-2-carbonyl)-6-oxo-hexahydro-pyrrolo[3,2-b]pyrrole-1-carbonyl]-3-methyl-butyl}-amide;
1452. 5-Phenyl-thiophene-2-carboxylic acid-{1-[4-(thiophene-2-carbonyl)-6-oxo-hexahydro-pyrrolo[3,2-b]pyrrole-1-carbonyl]-3-methyl-butyl}-amide;
1453. 5-Phenyl-thiophene-2-carboxylic acid-{1-[4-(5-chloro-thiophene-2-carbonyl)-6-oxo-hexahydro-pyrrolo[3,2-b]pyrrole-1-carbonyl]-3-methyl-butyl}-amide;
1454. 5-Phenyl-thiophene-2-carboxylic acid-{1-[4-(furan-3-carbonyl)-6-oxo-hexahydro-pyrrolo[3,2-b]pyrrole-1-carbonyl]-3-methyl-butyl}-amide;
1455. 5-Phenyl-thiophene-2-carboxylic acid-{1-[4-(thiophene-3-carbonyl)-6-oxo-hexahydro-pyrrolo[3,2-b]pyrrole-1-carbonyl]-3-methyl-butyl}-amide;
1456. 5-Phenyl-thiophene-2-carboxylic acid-[1-(4-cyclopentanecarbonyl-6-oxo-hexahydro-pyrrolo[3,2-b]pyrrole-1-carbonyl]-3-methyl-butyl)-amide;
1457. 5-Phenyl-thiophene-2-carboxylic acid-[1-(4-cyclohexanecarbonyl-6-oxo-hexahydro-pyrrolo[3,2-b]pyrrole-1-carbonyl)-3-methyl-butyl]-amide;
1458. 5-Phenyl-thiophene-2-carboxylic acid-{1-[4-(cyclopent-3-enecarbonyl)-6-oxo-hexahydro-pyrrolo[3,2-b]pyrrole-1-carbonyl]-3-methyl-butyl}-amide;
1459. 5-Phenyl-thiophene-2-carboxylic acid-{1-[4-(2-cyclopentyl-acetyl)-6-oxo-hexahydro-pyrrolo[3,2-b]pyrrole-1-carbonyl]-3-methyl-butyl}-amide;
1460. 5-Phenyl-thiophene-2-carboxylic acid-{1-[4-(2-cyclohexyl-acetyl)-6-oxo-hexahydro-pyrrolo[3,2-b]pyrrole-1-carbonyl]-3-methyl-butyl}-amide;
1461. 5-Phenyl-thiophene-2-carboxylic acid-{3-methyl-1-[6-oxo-4-(pyrrolidine-2-carbonyl)-hexahydro-pyrrolo[3,2-b]pyrrole-1-carbonyl]-butyl}-amide;
1462. 5-Phenyl-thiophene-2-carboxylic acid-{1-[4-(1-Acetyl-pyrrolidine-2-carbonyl)-6-oxo-hexahydro-pyrrolo[3,2-b]pyrrole-1-carbonyl]-3-methyl-butyl}-amide;
1463. 5-Phenyl-thiophene-2-carboxylic acid-{3-methyl-1-[6-oxopiperidine-2-carbonyl)-hexahydro-pyrrolo[3,2-b]pyrrole-1-carbonyl]-butyl}-amide;
1464. 5-Phenyl-thiophene-2-carboxylic acid-{1-[4-(1-Acetyl-piperidine-2-carbonyl)-6-oxo-hexahydro-pyrrolo[3,2-b]pyrrole-1-carbonyl]-3-methyl-butyl}-amide;
1465. 5-Phenyl-thiophene-2-carboxylic acid-{3-methyl-1-[6-oxo-4-(tetrahydro-furan-2-carbonyl)-hexahydro-pyrrolo[3,2-b]pyrrole-1-carbonyl]-butyl}-amide;
1466. 5-Phenyl-thiophene-2-carboxylic acid-{1-[4-(1-Amino-cyclobutanecarbonyl)-6-oxo-hexahydro-pyrrolo[3,2-b]pyrrole-1-carbonyl]-3-methyl-butyl}-amide;
1467. 5-Phenyl-thiophene-2-carboxylic acid-{1-[4-(1-Amino-cyclopentanecarbonyl)-6-oxo-hexahydro-pyrrolo[3,2-b]pyrrole-1-carbonyl]-3-methyl-butyl}-amide;
1468. 5-Phenyl-thiophene-2-carboxylic acid-{1-[4-(1-Amino-cyclohexanecarbonyl)-6-oxo-hexahydro-pyrrolo[3,2-b]pyrrole-1-carbonyl]-3-methyl-butyl}-amide;
1469. 5-Phenyl-thiophene-2-carboxylic acid-{1-[4-(1-Acetylamino-cyclobutanecarbonyl)-6-oxo-hexahydro-pyrrolo[3,2-b]pyrrole-1-carbonyl]-3-methyl-butyl}-amide;
1470. 5-Phenyl-thiophene-2-carboxylic acid-{1-[4-(1-Acetylamino-cyclopentanecarbonyl)-6-oxo-hexahydro-pyrrolo[3,2-b]pyrrole-1-carbonyl]-3-methyl-butyl}-amide;
1471. 5-Phenyl-thiophene-2-carboxylic acid-{1-[4-(1-Acetylamino-cyclohexanecarbonyl)-6-oxo-hexahydro-pyrrolo[3,2-b]pyrrole-1-carbonyl]-3-methyl-butyl}-amide;
1472. 5-Phenyl-thiophene-2-carboxylic acid-{1-[4-(1-hydroxy-cyclobutanecarbonyl)-6-oxo-hexahydro-pyrrolo[3,2-b]pyrrole-1-carbonyl]-3-methyl-butyl}-amide;
1473. 5-Phenyl-thiophene-2-carboxylic acid-{1-[4-(1-hydroxy-cyclopentanecarbonyl)-6-oxo-hexahydro-pyrrolo[3,2-b]pyrrole-1-carbonyl]-3-methyl-butyl}-amide;
1474. 5-Phenyl-thiophene-2-carboxylic acid-{1-[4-(1-hydroxy-cyclohexanecarbonyl)-6-oxo-hexahydro-pyrrolo[3,2-b]pyrrole-1-carbonyl]-3-methyl-butyl}-amide;
1475. 5-Phenyl-thiophene-2-carboxylic acid-{1-[4-(1-methoxy-cyclobutanecarbonyl)-6-oxo-hexahydro-pyrrolo[3,2-b]pyrrole-1-carbonyl]-3-methyl-butyl}-amide;
1476. 5-Phenyl-thiophene-2-carboxylic acid-{1-[4-(1-methoxy-cyclopentanecarbonyl)-6-oxo-hexahydro-pyrrolo[3,2-b]pyrrole-1-carbonyl]-3-methyl-butyl}-amide;
1477. 5-Phenyl-thiophene-2-carboxylic acid-{1-[4-(1-methoxy-cyclohexanecarbonyl)-6-oxo-hexahydro-pyrrolo[3,2-b]pyrrole-1-carbonyl]-3-methyl-butyl}-amide;
1478. 5-Phenyl-thiophene-2-carboxylic acid-{1-[4-(2-Amino-2-cyclopentyl-acetyl)-6-oxo-hexahydro-pyrrolo[3,2-b]pyrrole-1-carbonyl]-3-methyl-butyl}-amide;
1479. 5-Phenyl-thiophene-2-carboxylic acid-{1-[4-(2-Amino-2-cyclohexyl-acetyl)-6-oxo-hexahydro-pyrrolo[3,2-b]pyrrole-1-carbonyl]-3-methyl-butyl}-amide;
1480. 5-Phenyl-thiophene-2-carboxylic acid-{1-[4-(2-Acetylamino-2-cyclopentyl-acetyl)-6-oxo-hexahydro-pyrrolo[3,2-b]pyrrole-1-carbonyl]-3-methyl-butyl}-amide;
1481. 5-Phenyl-thiophene-2-carboxylic acid-{1-[4-(2-Acetylamino-2-cyclohexyl-acetyl)-6-oxo-hexahydro-pyrrolo[3,2-b]pyrrole-1-carbonyl]-3-methyl-butyl}-amide;
1482. 5-Phenyl-thiophene-2-carboxylic acid-{1-[4-(2-Acetylamino-propionyl)-6-oxo-hexahydro-pyrrolo[3,2-b]pyrrole-1-carbonyl]-3-methyl-butyl}-amide;
1483. 5-Phenyl-thiophene-2-carboxylic acid-{1-[4-(2-Acetylamino-acetyl)-6-oxo-hexahydro-pyrrolo[3,2-b]pyrrole-1-carbonyl]-3-methyl-butyl}-amide;
1484. 5-Phenyl-thiophene-2-carboxylic acid-(1-{4-[2-(Acetyl-methyl-amino)-acetyl]-6-oxo-hexahydro-pyrrolo[3,2-b]pyrrole-1-carbonyl}-3-methyl-butyl)-amide;
1485. 5-Phenyl-thiophene-2-carboxylic acid-{1-[4-(2-dimethylamino-acetyl)-6-oxo-hexahydro-pyrrolo[3,2-b]pyrrole-1-carbonyl]-3-methyl-butyl}-amide;
1486. 5-Phenyl-thiophene-2-carboxylic acid-{1-[4-(2-Amino-butyryl)-6-oxo-hexahydro-pyrrolo[3,2-b]pyrrole-1-carbonyl)-3-methyl-butyl}-amide;
1487. 5-Phenyl-thiophene-2-carboxylic acid-{1-[4-(2-Acetylamino-butyryl)-6-oxo-hexahydro-pyrrolo[3,2-b]pyrrole-1-carbonyl]-3-methyl-butyl}-amide;
1488. 5-Phenyl-thiophene-2-carboxylic acid-{1-[4-(2-amino-3-methyl-butyryl)-6-oxo-hexahydro-pyrrolo[3,2-b]pyrrole-1-carbonyl]-3-methyl-butyl}-amide;
1489. 5-Phenyl-thiophene-2-carboxylic acid-{1-[4-(2-Acetylamino-3-methyl-butyryl)-6-oxo-hexahydro-pyrrolo[3,2-b]pyrrole-1-carbonyl]-3-methyl-butyl}-amide;
1490. 5-Phenyl-thiophene-2-carboxylic acid-{1-[4-(2-amino-3,3-dimethyl-butyryl)-6-oxo-hexahydro-pyrrolo[3,2-b]pyrrole-1-carbonyl]-3-methyl-butyl}-amide;
1491. 5-Phenyl-thiophene-2-carboxylic acid-{1-[4-(2-Acetylamino-3,3-dimethyl-butyryl)-6-oxo-hexahydro-pyrrolo[3,2-b]pyrrole-1-carbonyl]-3-methyl-butyl}-amide;
1492. 5-Phenyl-thiophene-2-carboxylic acid-{1-[4-(2-Amino-3-methyl-pentanoyl)-6-oxo-hexahydro-pyrrolo[3,2-b]pyrrole-1-carbonyl]-3-methyl-butyl}-amide;
1493. 5-Phenyl-thiophene-2-carboxylic acid-{1-[4-(2-Acetylamino-3-methyl-pentanoyl)-6-oxo-hexahydro-pyrrolo[3,2-b]pyrrole-1-carbonyl]-3-methyl-butyl}-amide;
1494. 5-Phenyl-thiophene-2-carboxylic acid-[3-methyl-1-(6-oxo-4-pentanoyl-hexahydro-pyrrolo[3,2-b]pyrrole-1-carbonyl)-butyl]-amide;
1495. 5-Phenyl-thiophene-2-carboxylic acid-{3-methyl-1-[4-(3-methyl-pentanoyl)-6-oxo-hexahydro-pyrrolo[3,2-b]pyrrole-1-carbonyl]-butyl}-amide;
1496. 5-Phenyl-thiophene-2-carboxylic acid-{3-methyl-1-[4-(4-methyl-pentanoyl)-6-oxo-hexahydro-pyrrolo[3,2-b]pyrrole-1-carbonyl]-butyl}-amide;
1497. 5-Phenyl-thiophene-2-carboxylic acid-{1-[4-(2-Amino-4-methyl-pentanoyl)-6-oxo-hexahydro-pyrrolo[3,2-b]pyrrole-1-carbonyl]-3-methyl-butyl}-amide;
1498. 5-Phenyl-thiophene-2-carboxylic acid-{1-[4-(2-Acetylamino-4-methyl-pentanoyl)-6-oxo-hexahydro-pyrrolo[3,2-b]pyrrole-1-carbonyl]-3-methyl-butyl}-amide;
1499. 5-Phenyl-thiophene-2-carboxylic acid-{1-[4-(2-Amino-4,4-dimethyl-pentanoyl)-6-oxo-hexahydro-pyrrolo[3,2-b]pyrrole-1-carbonyl]-3-methyl-butyl}-amide;
1500. 5-Phenyl-thiophene-2-carboxylic acid-{1-[4-(2-Acetylamino-4,4-dimethyl-pentanoyl)-6-oxo-hexahydro-pyrrolo[3,2-b]pyrrole-1-carbonyl]-3-methyl-butyl}-amide;
1501. 5-Phenyl-thiophene-2-carboxylic acid-{1-[4-(2-Amino-pentanoyl)-6-oxo-hexahydro-pyrrolo[3,2-b]pyrrole-1-carbonyl]-3-methyl-butyl}-amide;
1502. 5-Phenyl-thiophene-2-carboxylic acid {1-[4-(2-Acetylamino-pentanoyl)-6-oxo-hexahydro-pyrrolo[3,2-b]pyrrole-1-carbonyl]-3-methyl-butyl}-amide;
1503. 5-Phenyl-thiophene-2-carboxylic acid-{1-[4-(2-Amino-5-methyl-hexanoyl)-6-oxo-hexahydro-pyrrolo[3,2-b]pyrrole-1-carbonyl]-3-methyl-butyl}-amide;
1504. 5-Phenyl-thiophene-2-carboxylic acid-{1-[4-(2-Acetylamino-5-methyl-hexanoyl)-6-oxo-hexahydro-pyrrolo[3,2-b]pyrrole-1-carbonyl]-3-methyl-butyl}-amide;
1505. 5-Phenyl-thiophene-2-carboxylic acid-{1-[4-(2-hydroxy-3-methyl-butyryl)-6-oxo-hexahydro-pyrrolo[3,2-b]pyrrole-1-carbonyl]-3-methyl-butyl}-amide;
1506. 5-Phenyl-thiophene-2-carboxylic acid-{1-[4-(2-methoxy-3-methyl-butyryl)-6-oxo-hexahydro-pyrrolo[3,2-b]pyrrole-1-carbonyl]-3-methyl-butyl}-amide;
1507. 5-Phenyl-thiophene-2-carboxylic acid-{1-[4-(2-hydroxy-3,3-dimethyl-butyryl)-6-oxo-hexahydro-pyrrolo[3,2-b]pyrrole-1-carbonyl]-3-methyl-butyl}-amide;
1508. 5-Phenyl-thiophene-2-carboxylic acid-{1-[4-(2-methoxy-3,3-dimethyl-butyryl)-6-oxo-hexahydro-pyrrolo[3,2-b]pyrrole-1-carbonyl]-3-methyl-butyl}-amide;
1509. 5-Phenyl-thiophene-2-carboxylic acid-{1-[4-(2-hydroxy-3-methyl-pentanoyl)-6-oxo-hexahydro-pyrrolo[3,2-b]pyrrole-1-carbonyl]-3-methyl-butyl}-amide;
1510. 5-Phenyl-thiophene-2-carboxylic acid-{1-[4-(2-methoxy-3-methyl-pentanoyl)-6-oxo-hexahydro-pyrrolo[3,2-b]pyrrole-1-carbonyl]-3-methyl-butyl}-amide;
1511. 5-Phenyl-thiophene-2-carboxylic acid-{1-[4-(2-hydroxy-4-methyl-pentanoyl)-6-oxo-hexahydro-pyrrolo[3,2-b]pyrrole-1-carbonyl]-3-methyl-butyl}-amide;
1512. 5-Phenyl-thiophene-2-carboxylic acid-{1-[4-(2-methoxy-4-methyl-pentanoyl)-6-oxo-hexahydro-pyrrolo[3,2-b]pyrrole-1-carbonyl]-3-methyl-butyl}-amide;
1513. 5-Phenyl-thiophene-2-carboxylic acid-{1-[4-(2-hydroxy-4,4-dimethyl-pentanoyl)-6-oxo-hexahydro-pyrrolo[3,2-b]pyrrole-1-carbonyl]-3-methyl-butyl}-amide;
1514. 5-Phenyl-thiophene-2-carboxylic acid-{1-[4-(2-methoxy-4,4-dimethyl-pentanoyl)-6-oxo-hexahydro-pyrrolo[3,2-b]pyrrole-1-carbonyl]-3-methyl-butyl}-amide;
1515. 5-Phenyl-thiophene-2-carboxylic acid {1-[4-(2-hydroxy-pentanoyl)-6-oxo-hexahydro-pyrrolo[3,2-b]pyrrole-1-carbonyl]-3-methyl-butyl}-amide;
1516. 5-Phenyl-thiophene-2-carboxylic acid-{1-[4-(2-methoxy-pentanoyl)-6-oxo-hexahydro-pyrrolo[3,2-b]pyrrole-1-carbonyl]-3-methyl-butyl}-amide;
1517. 5-Phenyl-thiophene-2-carboxylic acid-{1-[4-(2-hydroxy-5-methyl-hexanoyl)-6-oxo-hexahydro-pyrrolo[3,2-b]pyrrole-1-carbonyl]-3-methyl-butyl}-amide;
1518. 5-Phenyl-thiophene-2-carboxylic acid-{1-[4-(2-methoxy-5-methyl-hexanoyl)-6-oxo-hexahydro-pyrrolo[3,2-b]pyrrole-1-carbonyl]-3-methyl-butyl}-amide;
1519. N-[1-(4-Benzoyl-6-oxo-hexahydro-pyrrolo[3,2-b]pyrrole-1-carbonyl)-3-methyl-butyl]-4-pyrrolidin-1-ylbenzamide;
1520. N-{3-methyl-1-[6-oxo-4-(pyridine-2-carbonyl)hexahydro-pyrrolo[3,2-b]pyrrole-1-carbonyl]-butyl}-4-pyrrolidin-1-yl-benzamide;
1521. N-{3-methyl-1-[6-oxo-4-(1-oxy-pyridine-2-carbonyl)-hexahydro-pyrrolo[3,2-b]pyrrole-1-carbonyl]-butyl}-4-pyrrolidin-1-yl-benzamide;
1522. N-{3-methyl-1-[6-oxo-4-(pyridine-3-carbonyl)-hexahydro-pyrrolo[3,2-b]pyrrole-1-carbonyl]-butyl}-4-pyrrolidin-1-yl-benzamide;
1523. N-{3-methyl-1-[6-oxo-4-(1-oxy-pyridine-3-carbonyl)-hexahydro-pyrrolo[3,2-b]pyrrole-1-carbonyl]-butyl}-4-pyrrolidin-1-yl-benzamide;
1524. N-{3-methyl-1-[6-oxo-4-(pyridinecarbonyl)-hexahydro-pyrrolo[3,2-b]pyrrole-1-carbonyl]-butyl}-4-pyrrolidin-1-yl-benzamide;
1525. N-{3-methyl-1-[6-oxo-4-(1-oxy-pyridinecarbonyl)-hexahydro-pyrrolo[3,2-b]pyrrole-1-carbonyl]-butyl}-4-pyrrolidin-1-yl-benzamide;
1526. N-[1-(4-Benzenesulfonyl-6-oxo-hexahydro-pyrrolo[3,2-b]pyrrole-1-carbonyl)-3-methyl-butyl]-4-pyrrolidin-1-yl-benzamide;
1527. N-{3-methyl-1-[6-oxo-4-(pyridine-2-sulfonyl)-hexahydro-pyrrolo[3,2-b]pyrrole-1-carbonyl]-butyl}-4-pyrrolidin-1-yl-benzamide;
1528. N-{3-methyl-1-[6-oxo-4-(1-oxy-pyridine-2-sulfonyl)-hexahydro-pyrrolo[3,2-b]pyrrole-1-carbonyl]-butyl}-4-pyrrolidin-1-yl-benzamide;
1529. N-{3-methyl-1-[6-oxo-4-(pyridine-3-sulfonyl)-hexahydro-pyrrolo[3,2-b]pyrrole-1-carbonyl]-butyl}-4-pyrrolidin-1-yl-benzamide;
1530. N-{3-methyl-1-[6-oxo-4-(1-oxy-pyridine-3-sulfonyl)-hexahydro-pyrrolo[3,2-b]pyrrole-1-carbonyl]-butyl}-4-pyrrolidin-1-yl-benzamide;
1531. N-{3-methyl-1-[6-oxo-4-(pyridine-4-sulfonyl)-hexahydro-pyrrolo[3,2-b]pyrrole-1-carbonyl]-butyl}-4-pyrrolidin-1-yl-benzamide;
1532. N-{3-methyl-1-[6-oxo-4-(1-oxy-pyridine-4-sulfonyl)-hexahydro-pyrrolo[3,2-b]pyrrole-1-carbonyl]-butyl}-4-pyrrolidin-1-yl-benzamide;
1533. N-[3-methyl-1-(6-oxo-4-phenylacetyl-hexahydro-pyrrolo[3,2-b]pyrrole-1-carbonyl)-butyl]-4-pyrrolidin-1-yl-benzamide;
1534. 4-[2-(4-pyrrolidin-1-yl-benzoylamino)-4-methyl-pentanoyl]-3-oxo-hexahydro-pyrrolo[3,2-b]pyrrole-1-carboxylic acid phenylamide;
1535. 4-[2-(4-pyrrolidin-1-yl-benzoylamino)-4-methyl-pentanoyl]-3-oxo-hexahydro-pyrrolo[3,2-b]pyrrole-1-carboxylic acid isobutyl-amide;
1536. 4-[2-(4-pyrrolidin-1-yl-benzoylamino)-4-methyl-pentanoyl]-3-oxo-hexahydro-pyrrolo[3,2-b]pyrrole-1-carboxylic acid phenyl ester,
1537. 4-[2-(4-pyrrolidin-1-yl-benzoylamino)-4-methyl-pentanoyl]-3-oxo-hexahydro-pyrrolo[3,2-b]pyrrole-1-carboxylic acid isobutyl ester;
1538. N-{3-methyl-1-[6-oxo-4-(pyrrolidine-1-carbonyl)-hexahydro-pyrrolo[3,2-b]pyrrole-1-carbonyl]-butyl}-4-pyrrolidin-1-yl-benzamide;
1539. N-{3-methyl-1-[6-oxo-4-(piperidine-1-carbonyl)-hexahydro-pyrrolo[3,2-b]pyrrole-1-carbonyl]-butyl}-4-pyrrolidin-1-yl-benzamide;
1540. N-{3-methyl-1-[4-(morpholinecarbonyl)-6-oxo-hexahydro-pyrrolo[3,2-b]pyrrole-1-carbonyl]-butyl}-4-pyrrolidin-1-yl-benzamide;
1541. N-{3-methyl-1-[6-oxo-4-(piperazine-1-carbonyl)-hexahydro-pyrrolo[3,2-b]pyrrole-1-carbonyl]-butyl}-4-pyrrolidin-1-yl-benzamide;
1542. N-{3-methyl-1-[4-(4-methyl-piperazine-1-carbonyl)-6-oxo-hexahydro-pyrrolo[3,2-b]pyrrole-1-carbonyl]-butyl}-4-pyrrolidin-1-yl-benzamide;
1543. N-(3-methyl-1-{4-[2-(4-methyl-piperazin-1-yl)-acetyl]-6-oxo-hexahydro-pyrrolo[3,2-b]pyrrole-1-carbonyl}-butyl)-4-pyrrolidin-1-yl-benzamide;
1544. N-{3-methyl-1-[6-oxo-4-(2-piperazin-1-yl-acetyl)-hexahydro-pyrrolo[3,2-b]pyrrole-1-carbonyl]-butyl}-4-pyrrolidin-1-yl-benzamide;
1545. N-{1-[4-(furan-2-carbonyl)-6-oxo-hexahydro-pyrrolo[3,2-b]pyrrole-1-carbonyl]-3-methyl-butyl}-4-pyrrolidin-1-yl-benzamide;
1546. N-{1-[4-(5-chloro-furan-2-carbonyl)-6-oxo-hexahydro-pyrrolo[3,2-b]pyrrole-1-carbonyl]-3-methyl-butyl}-4-pyrrolidin-1-yl-benzamide;
1547. N-{1-[4-(thiophene-2-carbonyl)-6-oxo-hexahydro-pyrrolo[3,2-b]pyrrole-1-carbonyl]-3-methyl-butyl}-4-pyrrolidin-1-yl-benzamide;
1548. N-{1-[4-(5-chloro-thiophene-2-carbonyl)-6-oxo-hexahydro-pyrrolo[3,2-b]pyrrole-1-carbonyl]-3-methyl-butyl}-4-pyrrolidin-1-yl-benzamide;
1549. N-{1-[4-(furan-3-carbonyl)-6-oxo-hexahydro-pyrrolo[3,2-b]pyrrole-1-carbonyl]-3-methyl-butyl}-4-pyrrolidin-1-yl-benzamide;
1550. N-{1-[4-(thiophene-3-carbonyl)-6-oxo-hexahydro-pyrrolo[3,2-b]pyrrole-1-carbonyl]-3-methyl-butyl}-4-pyrrolidin-1-yl-benzamide;
1551. N-[1-(4-cyclopentanecarbonyl-6-oxo-hexahydro-pyrrolo[3,2-b]pyrrole-1-carbonyl)-3-methyl-butyl]-4-pyrrolidin-1-yl-benzamide;
1552. N-[1-(4-cyclohexanecarbonyl-6-oxo-hexahydro-pyrrolo[3,2-b]pyrrole-1-carbonyl)-3-methyl-butyl]-4-pyrrolidin-1-yl-benzamide;
1553. N-{1-[4-(cyclopent-3-enecarbonyl)-6-oxo-hexahydro-pyrrolo[3,2-b]pyrrole-1-carbonyl]-3-methyl-butyl}-4-pyrrolidin-1-yl-benzamide;
1554. N-{1-[4-(2-cyclopentyl-acetyl)-6-oxo-hexahydro-pyrrolo[3,2-b]pyrrole-1-carbonyl]-3-methyl-butyl}-4-pyrrolidin-1-yl-benzamide;
1555. N-{1-[4-(2-cyclohexyl-acetyl)-6-oxo-hexahydro-pyrrolo[3,2-b]pyrrole-1-carbonyl]-3-methyl-butyl}-4-pyrrolidin-1-yl-benzamide;
1556. 4-Pyrrolidin-1-yl-N-{3-methyl-1-[6-oxo-4-(pyrrolidine-2-carbonyl)-hexahydro-pyrrolo[3,2-b]pyrrole-1-carbonyl]-butyl}-benzamide;
1557. N-{1-[4-(1-Acetyl-pyrrolidine-2-carbonyl)-6-oxo-hexahydro-pyrrolo[3,2-b]pyrrole-1-carbonyl]-3-methyl-butyl}-4-pyrrolidin-1-yl-benzamide;
1558. N-{3-methyl-1-[6-oxo-4-(piperidine-2-carbonyl)-hexahydro-pyrrolo[3,2-b]pyrrole-1-carbonyl]-butyl}-4-pyrrolidin-1-yl-benzamide;
1559. N-{1-[4-(1-Acetyl-piperidine-2-carbonyl)-6-oxo-hexahydro-pyrrolo[3,2-b]pyrrole-1-carbonyl]-3-methyl-butyl}-4-pyrrolidin-1-yl-benzamide;
1560. N-{3-methyl-1-[6-oxo-4-(tetrahydro-furan-2-carbonyl)-hexahydro-pyrrolo[3,2-b]pyrrole-1-carbonyl]-butyl}-4-pyrrolidin-1-yl-benzamide;
1561. N-{1-[4-(1-Amino-cyclobutanecarbonyl)-6-oxo-hexahydro-pyrrolo[3,2-b]pyrrole-1-carbonyl]-3-methyl-butyl}-4-pyrrolidin-1-yl-benzamide;
1562. N-{1-[4-(1-Amino-cyclopentanecarbonyl)-6-oxo-hexahydro-pyrrolo[3,2-b]pyrrole-1-carbonyl]-3-methyl-butyl}-4-pyrrolidin-1-yl-benzamide;
1563. N-{1-[4-(1-Amino-cyclohexanecarbonyl)-6-oxo-hexahydro-pyrrolo[3,2-b]pyrrole-1-carbonyl]-3-methyl-butyl}-4-pyrrolidin-1-yl-benzamide;
1564. N-{1-[4-(1-Acetylamino-cyclobutanecarbonyl)-6-oxo-hexahydro-pyrrolo[3,2-b]pyrrole-1-carbonyl]-3-methyl-butyl}-4-pyrrolidin-1-yl-benzamide;
1565. N-{1-[4-(1-Acetylamino-cyclopentanecarbonyl)-6-oxo-hexahydro-pyrrolo[3,2-b]pyrrole-1-carbonyl]-3-methyl-butyl}-4-pyrrolidin-1-yl-benzamide;
1566. N-{1-[4-(1-Acetylamino-cyclohexanecarbonyl)-6-oxo-hexahydro-pyrrolo[3,2-b]pyrrole-1-carbonyl]-3-methyl-butyl}-4-pyrrolidin-1-yl-benzamide;
1567. N-{1-[4-(1-hydroxy-cyclobutanecarbonyl)-6-oxo-hexahydro-pyrrolo[3,2-b]pyrrole-1-carbonyl]-3-methyl-butyl}-4-pyrrolidin-1-yl-benzamide;
1568. N-{1-[4-(1-hydroxy-cyclopentanecarbonyl)-6-oxo-hexahydro-pyrrolo[3,2-b]pyrrole-1-carbonyl]-3-methyl-butyl}-4-pyrrolidin-1-yl-benzamide;
1569. N-{1-[4-(1-hydroxy-cyclohexanecarbonyl)-6-oxo-hexahydro-pyrrolo[3,2-b]pyrrole-1-carbonyl]-3-methyl-butyl}-4-pyrrolidin-1-yl-benzamide;
1570. N-{1-[4-(1-methoxy-cyclobutanecarbonyl)-6-oxo-hexahydro-pyrrolo[3,2-b]pyrrole-1-carbonyl]-3-methyl-butyl}-4-pyrrolidin-1-yl-benzamide;
1571. N-{1-[4-(1-methoxy-cyclopentanecarbonyl)-6-oxo-hexahydro-pyrrolo[3,2-b]pyrrole-1-carbonyl]-3-methyl-butyl}-4-pyrrolidin-1-yl-benzamide;
1572. N-{1-[4-(1-methoxy-cyclohexanecarbonyl)-6-oxo-hexahydro-pyrrolo[3,2-b]pyrrole-1-carbonyl]-3-methyl-butyl}-4-pyrrolidin-1-yl-benzamide;
1573. N-{1-[4-(2-Amino-2-cyclopentyl-acetyl)-6-oxo-hexahydro-pyrrolo[3,2-b]pyrrole-1-carbonyl]-3-methyl-butyl}-4-pyrrolidin-1-yl-benzamide;
1574. N-{1-[4-(2-Amino-2-cyclohexyl-acetyl)-6-oxo-hexahydro-pyrrolo[3,2-b]pyrrole-1-carbonyl]-3-methyl-butyl}-4-pyrrolidin-1-yl-benzamide;
1575. N-{1-[4-(2-Acetylamino-2-cyclopentyl-acetyl)-6-oxo-hexahydro-pyrrolo[3,2-b]pyrrole-1-carbonyl]-3-methyl-butyl}-4-pyrrolidin-1-yl-benzamide;
1576. N-{1-[4-(2-Acetylamino-2-cyclohexyl-acetyl)-6-oxo-hexahydro-pyrrolo[3,2-b]pyrrole-1-carbonyl]-3-methyl-butyl}-4-pyrrolidin-1-yl-benzamide;
1577. N-{1-[4-(2-Acetylamino-propionyl)-6-oxo-hexahydro-pyrrolo[3,2-b]pyrrole-1-carbonyl]-3-methyl-butyl}-4-pyrrolidin-1-yl-benzamide;
1578. N-{1-[4-(2-Acetylamino-acetyl)-6-oxo-hexahydro-pyrrolo[3,2-b]pyrrole-1-carbonyl]-3-methyl-butyl}-4-pyrrolidin-1-yl-benzamide;
1579. N-(1-{4-[2-(Acetyl-methyl-amino)-acetyl]-6-oxo-hexahydro-pyrrolo[3,2-b]pyrrole-1-carbonyl}-3-methyl-butyl]-4-pyrrolidin-1-yl-benzamide;
1580. N-{1-[4-(2-dimethylamino-acetyl)-6-oxo-hexahydro-pyrrolo[3,2-b]pyrrole-1-carbonyl]-3-methyl-butyl}-4-pyrrolidin-1-yl-benzamide;
1581. N-{1-[4-(2-Amino-butyryl)-6-oxo-hexahydro-pyrrolo[3,2-b]pyrrole-1-carbonyl]-3-methyl-butyl}-4-pyrrolidin-1-yl-benzamide;
1582. N-{1-[4-(2-Acetylamino-butyryl)-6-oxo-hexahydro-pyrrolo[3,2-b]pyrrole-1-carbonyl]-3-methyl-butyl}-4-pyrrolidin-1-yl-benzamide;
1583. N-{1-[4-(2-amino-3-methyl-butyryl)-6-oxo-hexahydro-pyrrolo[3,2-b]pyrrole-1-carbonyl]-3-methyl-butyl}-4-pyrrolidin-1-yl-benzamide;
1584. N-{1-[4-(2-Acetylamino-3-methyl-butyryl)-6-oxo-hexahydro-pyrrolo[3,2-b]pyrrole-1-carbonyl]-3-methyl-butyl}-4-pyrrolidin-1-yl-benzamide;
1585. N-{1-[4-(2-amino-3,3-dimethyl-butyryl)-6-oxo-hexahydro-pyrrolo[3,2-b]pyrrole-1-carbonyl]-3-methyl-butyl}-4-pyrrolidin-1-yl-benzamide;
1586. N-{1-[4-(2-Acetylamino-3,3-dimethyl-butyryl)-6-oxo-hexahydro-pyrrolo[3,2-b]pyrrole-1-carbonyl]-3-methyl-butyl}-4-pyrrolidin-1-yl-benzamide;
1587. N-{1-[4-(2-Amino-3-methyl-pentanoyl)-6-oxo-hexahydro-pyrrolo[3,2-b]pyrrole-1-carbonyl]-3-methyl-butyl}-4-pyrrolidin-1-yl-benzamide;
1588. N-{1-[4-(2-Acetylamino-3-methyl-pentanoyl)-6-oxo-hexahydro-pyrrolo[3,2-b]pyrrole-1-carbonyl]-3-methyl-butyl}-4-pyrrolidin-1-yl-benzamide;
1589. N-[3-methyl-1-(6-oxo-4-pentanoyl-hexahydro-pyrrolo[3,2-b]pyrrole-1-carbonyl)-butyl]-4-pyrrolidin-1-yl-benzamide;
1590. N-{3-methyl-1-[4-(3-methyl-pentanoyl)-6-oxo-hexahydro-pyrrolo[3,2-b]pyrrole-1-carbonyl]-butyl}-4-pyrrolidin-1-yl-benzamide;
1591. N-{3-methyl-1-[4-(4-methyl-pentanoyl)-6-oxo-hexahydro-pyrrolo[3,2-b]pyrrole-1-carbonyl]-butyl}-4-pyrrolidin-1-yl-benzamide;
1592. N-{1-[4-(2-Amino-4-methyl-pentanoyl)-6-oxo-hexahydro-pyrrolo[3,2-b]pyrrole-1-carbonyl]-3-methyl-butyl}-4-pyrrolidin-1-yl-benzamide;
1593. N-{1-[4-(2-Acetylamino-4-methyl-pentanoyl)-6-oxo-hexahydro-pyrrolo[3,2-b]pyrrole-1-carbonyl]-3-methyl-butyl}-4-pyrrolidin-1-yl-benzamide;
1594. N-{1-[4-(2-Amino-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
1595. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
1596. N-{1-[4-(2-Amino-pentanoyl)-6-oxo-hexahydro-pyrrolo[3,2-b]pyrrole-1-carbonyl]-3-methyl-butyl}-4-pyrrolidin-1-yl-benzamide;
1597. N-{1-[4-(2-Acetylamino-pentanoyl)-6-oxo-hexahydro-pyrrolo[3,2-b]pyrrole-1-carbonyl]-3-methyl-butyl}-4-pyrrolidin-1-yl-benzamide;
1598. N-{1-[4-(2-Amino-5-methyl-hexanoyl)-6-oxo-hexahydro-pyrrolo[3,2-b]pyrrole-1-carbonyl]-3-methyl-butyl}-4-pyrrolidin-1-yl-benzamide;
1599. N-{1-[4-(2-Acetylamino-5-methyl-hexanoyl)-6-oxo-hexahydro-pyrrolo[3,2-b]pyrrole-1-carbonyl]-3-methyl-butyl}-4-pyrrolidin-1-yl-benzamide;
1600. N-{1-[4-(2-hydroxy-3-methyl-butyryl)-6-oxo-hexahydro-pyrrolo[3,2-b]pyrrole-1-carbonyl]-3-methyl-butyl}-4-pyrrolidin-1-yl-benzamide;
1601. N-{1-[4-(2-methoxy-3-methyl-butyryl)-6-oxo-hexahydro-pyrrolo[3,2-b]pyrrole-1-carbonyl]-3-methyl-butyl}-4-pyrrolidin-1-yl-benzamide;
1602. N-{1-[4-(2-hydroxy-3,3-dimethyl-butyryl)-6-oxo-hexahydro-pyrrolo[3,2-b]pyrrole-1-carbonyl]-3-methyl-butyl}-4-pyrrolidin-1-yl-benzamide;
1603. N-{1-[4-(2-methoxy-3,3-dimethyl-butyryl)-6-oxo-hexahydro-pyrrolo[3,2-b]pyrrole-1-carbonyl]-3-methyl-butyl}-4-pyrrolidin-1-yl-benzamide;
1604. N-{1-[4-(2-hydroxy-3-methyl-pentanoyl)-6-oxo-hexahydro-pyrrolo[3,2-b]pyrrole-1-carbonyl]-3-methyl-butyl}-4-pyrrolidin-1-yl-benzamide;
1605. N-{1-[4-(2-methoxy-3-methyl-pentanoyl)-6-oxo-hexahydro-pyrrolo[3,2-b]pyrrole-1-carbonyl]-3-methyl-butyl}-4-pyrrolidin-1-yl-benzamide;
1606. N-{1-[4-(2-hydroxy-4-methyl-pentanoyl)-6-oxo-hexahydro-pyrrolo[3,2-b]pyrrole-1-carbonyl]-3-methyl-butyl}-4-pyrrolidin-1-yl-benzamide;
1607. N-{1-[4-(2-methoxymethyl-pentanoyl)-6-oxo-hexahydro-pyrrolo[3,2-b]pyrrole-1-carbonyl]-3-methyl-butyl}-4-pyrrolidin-1-yl-benzamide;
1608. N-{1-[4-(2-hydroxy-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
1609. N-{1-[4-(2-methoxy-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
1610. N-{1-[4-(2-hydroxy-pentanoyl)-6-oxo-hexahydro-pyrrolo[3,2-b]pyrrole-1-carbonyl]-3-methyl-butyl}-4-pyrrolidin-1-yl-benzamide;
1611. N-{1-[4-(2-methoxy-pentanoyl)-6-oxo-hexahydro-pyrrolo[3,2-b]pyrrole-1-carbonyl]-3-methyl-butyl}-4-pyrrolidin-1-yl-benzamide;
1612. N-{1-[4-(2-hydroxy-5-methyl-hexanoyl)-6-oxo-hexahydro-pyrrolo[3,2-b]pyrrole-1-carbonyl]-3-methyl-butyl}-4-pyrrolidin-1-yl-benzamide;
1613. N-{1-[4-(2-methoxy-5-methyl-hexanoyl)-6-oxo-hexahydro-pyrrolo[3,2-b]pyrrole-1-carbonyl]-3-methyl-butyl}-4-pyrrolidin-1-yl-benzamide;
1614. N-[1-(4-Benzoyl-6-oxo-hexahydro-pyrrolo[3,2-b]pyrrole-1-carbonyl)-3-methyl-butyl]-4-morpholin-4-ylbenzamide;
1615. N-{3-methyl-1-[6-oxo-4-(1-oxy-pyridine-2-carbonyl)-hexahydro-pyrrolo[3,2-b]pyrrole-1-carbonyl]-butyl}-4-morpholin-4-yl-4-yl-benzamide;
1616. N-{3-methyl-1-[6-oxo-4-(4-pyridine-3-carbonyl)-hexahydro-pyrrolo[3,2-b]pyrrole-1-carbonyl]-butyl}-4-morpholin-4-yl-benzamide;
1617. N-{3-methyl-1-[6-oxo-4-(1-oxy-pyridine-3-carbonyl)-hexahydro-pyrrolo[3,2-b]pyrrole-1-carbonyl]-butyl}-4-morpholin-4-yl-benzamide;
1618. N-{3-methyl-1-[6-oxo-4-(pyridine-4-carbonyl)-hexahydro-pyrrolo[3,2-b]pyrrole-1-carbonyl]-butyl}-4-morpholin-4-yl-benzamide;
1619. N-{3-methyl-1-[6-oxo-4-(1-oxy-pyridine-4-carbonyl)-hexahydro-pyrrolo[3,2-b]pyrrole-1-carbonyl]-butyl}-4-morpholin-4-yl-benzamide;
1620. N-[1-(4-Benzenesulfonyl-6-oxo-hexahydro-pyrrolo[3,2-b]pyrrole-1-carbonyl)-3-methyl-butyl]-4-morpholin-4-yl-benzamide;
1621. N-{3-methyl-1-[6-oxo-4-(pyridine-4-sulfonyl)-hexahydro-pyrrolo[3,2-b]pyrrole-1-carbonyl]-butyl}-4-morpholin-4-yl-benzamide;
1622. N-{3-methyl-1-[6-oxo-4-(1-oxy-pyridinesulfonyl)-hexahydro-pyrrolo[3,2-b]pyrrole-1-carbonyl]-butyl}-4-morpholin-4-yl-benzamide;
1623. N-[3-methyl-1-(6-oxo-4-phenylacetyl-hexahydro-pyrrolo[3,2-b]pyrrole-1-carbonyl)-butyl]-4-morpholin-4-yl-benzamide;
1624. 4-[2-(4-morpholin-4-yl-benzoylamino)-4-methyl-pentanoyl]-3-oxo-hexahydro-pyrrolo[3,2-b]pyrrole-1-carboxylic acid phenylamide;
1625. 4-[2-(4-morpholin-4-yl-benzoylamino)-4-methyl-pentanoyl]-3-oxo-hexahydro-pyrrolo[3,2-b]pyrrole-1-carboxylic acid isobutyl-amide;
1626. 4-[2-(4-morpholin-4-yl-benzoylamino)-4-methyl-pentanoyl]-3-oxo-hexahydro-pyrrolo[3,2-b]pyrrole-1-carboxylic acid phenyl ester;
1627. 4-[2-(4-morpholin-4-yl-benzoylamino)-4-methyl-pentanoyl]-3-oxo-hexahydro-pyrrolo[3,2-b]pyrrole-1-carboxylic acid isobutyl ester;
1628. N-{3-methyl-1-[6-oxo-4-(pyrrolidine-1-carbonyl)-hexahydro-pyrrolo[3,2-b]pyrrole-1-carbonyl]-butyl}-4-morpholin-4-yl-benzamide;
1629. N-{3-methyl-1-[6-oxo-4-(piperidine-1-carbonyl)-hexahydro-pyrrolo[3,2-b]pyrrole-1-carbonyl]-butyl}-4-morpholin-4-yl-benzamide;
1630. N-{3-methyl-1-[4-(morpholin-4-carbonyl)-6-oxo-hexahydro-pyrrolo[3,2-b]pyrrole-1-carbonyl]-butyl}-4-morpholin-4-yl-benzamide;
1631. N-{3-methyl-1-[6-oxo-4-(piperazine-1-carbonyl)-hexahydro-pyrrolo[3,2-b]pyrrole-1-carbonyl]-butyl}-4-morpholin-4-yl-benzamide;
1632. N-{3-methyl-1-[4-(4-methyl-piperazine-1-carbonyl)-6-oxo-hexahydro-pyrrolo[3,2-b]pyrrole-1-carbonyl]-butyl}-4-morpholin-4-yl-benzamide;
1633. N-(3-methyl-1-{4-[2-(4-methyl-piperazin-1-yl)-acetyl]-6-oxo-hexahydro-pyrrolo[3,2-b]pyrrole-1-carbonyl}-butyl)-4-morpholin-4-yl-benzamide;
1634. N-{3-methyl-1-[6-oxo-4-(2-piperazin-1-yl-acetyl)-hexahydro-pyrrolo[3,2-b]pyrrole-1-carbonyl]-butyl}-4-morpholin-4-yl-benzamide;
1635. N-{1-[4-(furan-2-carbonyl)-6-oxo-hexahydro-pyrrolo[3,2-b]pyrrole-1-carbonyl]-3-methyl-butyl}-4-morpholin-4-yl-benzamide;
1636. N-{1-[4-(5-chloro-furan-2-carbonyl)-6-oxo-hexahydro-pyrrolo[3,2-b]pyrrole-1-carbonyl]-3-methyl-butyl}-4-morpholin-4-yl-benzamide;
1637. N-{1-[4-(thiophene-2-carbonyl)-6-oxo-hexahydro-pyrrolo[3,2-b]pyrrole-1-carbonyl]-3-methyl-butyl}-4-morpholin-4-yl-benzamide;
1638. N-{1-[4-(5-chloro-thiophene-2-carbonyl)-6-oxo-hexahydro-pyrrolo[3,2-b]pyrrole-1-carbonyl]-3-methyl-butyl}-4-morpholin-4-yl-benzamide;
1639. N-{1-[4-(furan-3-carbonyl)-6-oxo-hexahydro-pyrrolo[3,2-b]pyrrole-1-carbonyl]-3-methyl-butyl}-4-morpholin-4-yl-benzamide;
1640. N-{1-[4-(thiophene-3-carbonyl)-6-oxo-hexahydro-pyrrolo[3,2-b]pyrrole-1-carbonyl]-3-methyl-butyl}-4-morpholin-4-yl-benzamide;
1641. N-[1-(4-cyclopentanecarbonyl-6-oxo-hexahydro-pyrrolo[3,2-b]pyrrole-1-carbonyl)-3-methyl-butyl]-4-morpholin-4-yl-benzamide;
1642. N-[1-(4-cyclohexanecarbonyl-6-oxo-hexahydro-pyrrolo[3,2-b]pyrrole-1-carbonyl)-3-methyl-butyl]-4-morpholin-4-yl-benzamide;
1643. N-{1-[4-(cyclopent-3-enecarbonyl)-6-oxo-hexahydro-pyrrolo[3,2-b]pyrrole-1-carbonyl]-3-methyl-butyl}-4-morpholin-4-yl-benzamide;
1644. N-{1-[4-(2-cyclopentyl-acetyl)-6-oxo-hexahydro-pyrrolo[3,2-b]pyrrole-1-carbonyl]-3-methyl-butyl}-4-morpholin-4-yl-benzamide;
1645. N-{1-[4-(2-cyclohexyl-acetyl)-6-oxo-hexahydro-pyrrolo[3,2-b]pyrrole-1-carbonyl]-3-methyl-butyl}-4-morpholin-4-yl-benzamide;
1646. 4-morpholin-4-yl-N-{3-methyl-1-[6-oxo-4-(pyrrolidine-2-carbonyl)-hexahydro-pyrrolo[3,2-b]pyrrole-1-carbonyl]-butyl}-benzamide;
1647. N-{1-[4-(1-Acetyl-pyrrolidine-2-carbonyl)-6-oxo-hexahydro-pyrrolo[3,2-b]pyrrole-1-carbonyl]-3-methyl-butyl}-4-morpholin-4-yl-benzamide;
1648. N-{3-methyl-1-[6-oxo-4-piperidine-2-carbonyl)-hexahydro-pyrrolo[3,2-b]pyrrole-1-carbonyl]-butyl}-4-morpholin-4-yl-benzamide;
1649. N-{1-[4-(1-Acetyl-piperidine-2-carbonyl)-6-oxo-hexahydro-pyrrolo[3,2-b]pyrrole-1-carbonyl]-3-methyl-butyl}-4-morpholin-4-yl-benzamide;
1650. N-{3-methyl-1-[6-oxo-4-(tetrahydro-furan-2-carbonyl)-hexahydro-pyrrolo[3,2-b]pyrrole-1-carbonyl]-butyl}-4-morpholin-4-yl-benzamide;
1651. N-{1-[4-(1-Amino-cyclobutanecarbonyl)-6-oxo-hexahydro-pyrrolo[3,2-b]pyrrole-1-carbonyl]-3-methyl-butyl}-4-morpholin-4-yl-benzamide;
1652. N-{1-[4-(1-Amino-cyclopentanecarbonyl)-6-oxo-hexahydro-pyrrolo[3,2-b]pyrrole-1-carbonyl]-3-methyl-butyl}-4-morpholin-4-yl-benzamide;
1653. N-{1-[4-(1-Amino-cyclohexanecarbonyl)-6-oxo-hexahydro-pyrrolo[3,2-b]pyrrole-1-carbonyl]-3-methyl-butyl}-4-morpholin-4-yl-benzamide;
1654. N-{1-[4-(1-Acetylamino-cyclobutanecarbonyl)-6-oxo-hexahydro-pyrrolo[3,2-b]pyrrole-1-carbonyl]-3-methyl-butyl}-4-morpholin-4-yl-benzamide;
1655. N-{1-[4-(1-Acetylamino-cyclopentanecarbonyl)-6-oxo-hexahydro-pyrrolo[3,2-b]pyrrole-1-carbonyl]-3-methyl-butyl}-4-morpholin-4-yl-benzamide;
1656. N-{1-[4-(1-Acetylamino-cyclohexanecarbonyl)-6-oxo-hexahydro-pyrrolo[3,2-b]pyrrole-1-carbonyl]-3-methyl-butyl}-4-morpholin-4-yl-benzamide;
1657. N-{1-[4-(1-hydroxy-cyclobutanecarbonyl)-6-oxo-hexahydro-pyrrolo[3,2-b]pyrrole-1-carbonyl]-3-methyl-butyl}-4-morpholin-4-yl-benzamide;
1658. N-{1-[4-(1-hydroxy-cyclopentanecarbonyl)-6-oxo-hexahydro-pyrrolo[3,2-b]pyrrole-1-carbonyl]-3-methyl-butyl}-4-morpholin-4-yl-benzamide;
1659. N-{1-[4-(1-hydroxy-cyclohexanecarbonyl)-6-oxo-hexahydro-pyrrolo[3,2-b]pyrrole-1-carbonyl]-3-methyl-butyl}-4-morpholin-4-yl-benzamide;
1660. N-{1-[4-(1-methoxy-cyclobutanecarbonyl)-6-oxo-hexahydro-pyrrolo[3,2-b]pyrrole-1-carbonyl]-3-methyl-butyl}-4-morpholin-4-yl-benzamide;
1661. N-{1-[4-(1-methoxy-cyclopentanecarbonyl)-6-oxo-hexahydro-pyrrolo[3,2-b]pyrrole-1-carbonyl]-3-methyl-butyl}-4-morpholin-4-yl-benzamide;
1662. N-{1-[4-(1-methoxy-cyclohexanecarbonyl)-6-oxo-hexahydro-pyrrolo[3,2-b]pyrrole-1-carbonyl]-3-methyl-butyl}-4-morpholin-4-yl-benzamide;
1663. N-{1-[4-(2-Amino-2-cyclopentyl-acetyl)-6-oxo-hexahydro-pyrrolo[3,2-b]pyrrole-1-carbonyl]-3-methyl-butyl}-4-morpholin-4-yl-benzamide;
1664. N-{1-[4-(2-Amino-2-cyclohexyl-acetyl)-6-oxo-hexahydro-pyrrolo[3,2-b]pyrrole-1-carbonyl]-3-methyl-butyl}-4-morpholin-4-yl-benzamide;
1665. N-{1-[4-(2-Acetylamino-2-cyclopentyl-acetyl)-6-oxo-hexahydro-pyrrolo[3,2-b]pyrrole-1-carbonyl]-3-methyl-butyl}-4-morpholin-4-yl-benzamide;
1666. N-{1-[4-(2-Acetylamino-2-cyclohexyl-acetyl)-6-oxo-hexahydro-pyrrolo[3,2-b]pyrrole-1-carbonyl]-3-methyl-butyl}-4-morpholin-4-yl-benzamide;
1667. N-{1-[4-(2-Acetylamino-propionyl)-6-oxo-hexahydro-pyrrolo[3,2-b]pyrrole-1-carbonyl]-3-methyl-butyl}-4-morpholin-4-yl-benzamide;
1668. N-{1-[4-(2-Acetylamino-acetyl)-6-oxo-hexahydro-pyrrolo[3,2-b]pyrrole-1-carbonyl]-3-methyl-butyl}-4-morpholin-4-yl-benzamide;
1669. N-(1-{4-[2-(Acetyl-methyl-amino)-acetyl]-6-oxo-hexahydro-pyrrolo[3,2-b]pyrrole-1-carbonyl}-3-methyl-butyl)-4-morpholin-4-yl-benzamide;
1670. N-{1-[4-(2-dimethylamino-acetyl)-6-oxo-hexahydro-pyrrolo[3,2-b]pyrrole-1-carbonyl]-3-methyl-butyl}-4-morpholin-4-yl-benzamide;
1671. N-{1-[4-(2-Amino-butyryl)-6-oxo-hexahydro-pyrrolo[3,2-b]pyrrole-1-carbonyl]-3-methyl-butyl}-4-morpholin-4-yl-benzamide;
1672. N-{1-[4-(2-Acetylamino-butyryl)-6-oxo-hexahydro-pyrrolo[3,2-b]pyrrole-1-carbonyl]-3-methyl-butyl}-4-morpholin-4-yl-benzamide;
1673. N-{1-[4-(2-amino-3-methyl-butyryl)-6-oxo-hexahydro-pyrrolo[3,2-b]pyrrole-1-carbonyl]-3-methyl-butyl}-4-morpholin-4-yl-benzamide;
1674. N-{1-[4-(2-Acetylamino-3-methyl-butyryl)-6-oxo-hexahydro-pyrrolo[3,2-b]pyrrole-1-carbonyl]-3-methyl-butyl}-4-morpholin-4-yl-benzamide;
1675. N-{1-[4-(2-amino-3,3-ethyl-butyryl)-6-oxo-hexahydro-pyrrolo[3,2-b]pyrrole-1-carbonyl]-3-methyl-butyl}-4-morpholin-4-yl-benzamide;
1676. N-{1-[4-(2-Acetylamino-3,3-dimethyl-butyryl)-6-oxo-hexahydro-pyrrolo[3,2-b]pyrrole-1-carbonyl]-3-methyl-butyl}-4-morpholin-4-yl-benzamide;
1677. N-{1-[4-(2-Amino-3-methyl-pentanoyl)-6-oxo-hexahydro-pyrrolo[3,2-b]pyrrole-1-carbonyl]-3-methyl-butyl}-4-morpholin-4-yl-benzamide;
1678. N-{1-[4-(2-Acetylamino-3-methyl-pentanoyl)-6-oxo-hexahydro-pyrrolo[3,2-b]pyrrole-1-carbonyl]-3-methyl-butyl}-4-morpholin-4-yl-benzamide;
1679. N-[3-methyl-1-(6-oxo-4-pentanoyl-hexahydro-pyrrolo[3,2-b]pyrrole-1-carbonyl)-butyl]-4-morpholin-4-yl-benzamide;
1680. N-{3-methyl-1-[4-(3-methyl-pentanoyl)-6-oxo-hexahydro-pyrrolo[3,2-b]pyrrole-1-carbonyl]-butyl}-4-morpholin-4-yl-benzamide;
1681. N-{3-methyl-1-[4-(4-methyl-pentanoyl)-6-oxo-hexahydro-pyrrolo[3,2-b]pyrrole-1-carbonyl]-butyl}-4-morpholin-4-yl-benzamide;
1682. N-{1-[4-(2-Amino-4-methyl-pentanoyl)-6-oxo-hexahydro-pyrrolo[3,2-b]pyrrole-1-carbonyl]-3-methyl-butyl}-4-morpholin-4-yl-benzamide;
1683. N-{1-[4-(2-Acetylamino-4-methyl-pentanoyl)-6-oxo-hexahydro-pyrrolo[3,2-b]pyrrole-1-carbonyl]-3-methyl-butyl}-4-morpholin-4-yl-benzamide;
1684. N-{1-[4-(2-Amino-4,4-dimethyl-pentanoyl)-6-oxo-hexahydro-pyrrolo[3,2-b]pyrrole-1-carbonyl]-3-methyl-butyl}-4-morpholin-4-yl-benzamide;
1685. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-morpholin-4-yl-benzamide;
1686. N-{1-[4-(2-Amino-pentanoyl)-6-oxo-hexahydro-pyrrolo[3,2-b]pyrrole-1-carbonyl]-3-methyl-butyl}-4-morpholin-4-yl-benzamide;
1687. N-{1-[4-(2-Acetylamino-pentanoyl)-6-oxo-hexahydro-pyrrolo[3,2-b]pyrrole-1-carbonyl]-3-methyl-butyl}-4-morpholin-4-yl-benzamide;
1688. N-{1-[4-(2-Amino-5-methyl-hexanoyl)-6-oxo-hexahydro-pyrrolo[3,2-b]pyrrole-1-carbonyl]-3-methyl-butyl}-4-morpholin-4-yl-benzamide;
1689. N-{1-[4-(2-Acetylamino-5-methyl-hexanoyl)-6-oxo-hexahydro-pyrrolo[3,2-b]pyrrole-1-carbonyl]-3-methyl-butyl}-4-morpholin-4-yl-benzamide;
1690. N-{1-[4-(2-hydroxy-3-methyl-butyryl)-6-oxo-hexahydro-pyrrolo[3,2-b]pyrrole-1-carbonyl]-3-methyl-butyl}-4-morpholin-4-yl-benzamide;
1691. N-{1-[4-(2-methoxy-3-methyl-butyryl)-6-oxo-hexahydro-pyrrolo[3,2-b]pyrrole-1-carbonyl]-3-methyl-butyl}-4-morpholin-4-yl-benzamide;
1692. N-{1-[4-(2-hydroxy-3,3-dimethyl-butyryl)-6-oxo-hexahydro-pyrrolo[3,2-b]pyrrole-1-carbonyl]-3-methyl-butyl}-4-morpholin-4-yl-benzamide;
1693. N-{1-[4-(2-methoxy-3,3-dimethyl-butyryl)-6-oxo-hexahydro-pyrrolo[3,2-b]pyrrole-1-carbonyl]-3-methyl-butyl}-4-morpholin-4-yl-benzamide;
1694. N-{1-[4-(2-hydroxy-3-methyl-pentanoyl)-6-oxo-hexahydro-pyrrolo[3,2-b]pyrrole-1-carbonyl]-3-methyl-butyl}-4-morpholin-4-yl-benzamide;
1695. N-{1-[4-(2-methoxy-3-methyl-pentanoyl)-6-oxo-hexahydro-pyrrolo[3,2-b]pyrrole-1-carbonyl]-3-methyl-butyl}-4-morpholin-4-yl-benzamide;
1696. N-{1-[4-(2-hydroxy methyl-pentanoyl)-6-oxo-hexahydro-pyrrolo[3,2-b]pyrrole-1-carbonyl]-3-methyl-butyl}-4-morpholin-4-yl-benzamide;
1697. N-{1-[4-(2-methoxy methyl-pentanoyl)-6-oxo-hexahydro-pyrrolo[3,2-b]pyrrole-1-carbonyl]-3-methyl-butyl}-4-morpholin-4-yl-benzamide;
1698. N-{1-[4-(2-hydroxy-4,4-dimethyl-pentanoyl)-6-oxo-hexahydro-pyrrolo[3,2-b]pyrrole-1-carbonyl]-3-methyl-butyl}-4-morpholin-4-yl-benzamide;
1699. N-{1-[4-(2-methoxy-4,4-dimethyl-pentanoyl)-6-oxo-hexahydro-pyrrolo[3,2-b]pyrrole-1-carbonyl]-3-methyl-butyl}-4-morpholin-4-yl-benzamide;
1700. N-{1-[4-(2-hydroxy-pentanoyl)-6-oxo-hexahydro-pyrrolo[3,2-b]pyrrole-1-carbonyl]-3-methyl-butyl}-4-morpholin-4-yl-benzamide;
1701. N-{1-[4-(2-methoxy-pentanoyl)-6-oxo-hexahydro-pyrrolo[3,2-b]pyrrole-1-carbonyl]-3-methyl-butyl}-4-morpholin-4-yl-benzamide;
1702. N-{1-[4-(2-hydroxy-5-methyl-hexanoyl)-6-oxo-hexahydro-pyrrolo[3,2-b]pyrrole-1-carbonyl]-3-methyl-butyl}-4-morpholin-4-yl-benzamide;
1703. N-{1-[4-(2-methoxy-5-methyl-hexanoyl)-6-oxo-hexahydro-pyrrolo[3,2-b]pyrrole-1-carbonyl]-3-methyl-butyl}-4-morpholin-4-yl-benzamide;
1704. N-[1-(4-Benzoyl-6-oxo-hexahydro-pyrrolo[3,2-b]pyrrole-1-carbonyl)-3-methyl-butyl]-4-piperazin-1-ylbenzamide;
1705. N-{3-methyl-1-[6-oxo-4-(pyridine-2-carbonyl)-hexahydro-pyrrolo[3,2-b]pyrrole-1-carbonyl]-butyl}-4-piperazin-1-yl-benzamide;
1706. N-{3-methyl-1-[6-oxo-4-(1-oxy-pyridine-2-carbonyl)-hexahydro-pyrrolo[3,2-b]pyrrole-1-carbonyl]-butyl})piperazin-1-yl-benzamide;
1707. N-{3-methyl-1-[6-oxo-4-(pyridine-3-carbonyl)-hexahydro-pyrrolo[3,2-b]pyrrole-1-carbonyl]-butyl}-4-piperazin-1-yl-benzamide;
1708. N-{3-methyl-1-[6-oxo-4-(1-oxy-pyridine-3-carbonyl)-hexahydro-pyrrolo[3,2-b]pyrrole-1-carbonyl]-butyl}-4-piperazin-1-yl-benzamide;
1709. N-{3-methyl-1-[6-oxo-4-(pyridine-4-carbonyl)-hexahydro-pyrrolo[3,2-b]pyrrole-1-carbonyl]-butyl}-4-piperazin-1-yl-benzamide;
1710. N-{3-methyl-1-[6-oxo-4-(1-oxy-pyridine-4-carbonyl)-hexahydro-pyrrolo[3,2-b]pyrrole-1-carbonyl]-butyl}-4-piperazin-1-yl-benzamide;
1711. N-[1-(4-Benzenesulfonyl-6-oxo-hexahydro-pyrrolo[3,2-b]pyrrole-1-carbonyl)-3-methyl-butyl]-4-piperazin-1-yl-benzamide;
1712. N-{3-methyl-1-[6-oxo-4-(pyridine-2-sulfonyl)-hexahydro-pyrrolo[3,2-b]pyrrole-1-carbonyl]-butyl}-4-piperazin-1-yl-benzamide;
1713. N-{3-methyl-1-[6-oxo-4-(1-oxy-pyridine-2-sulfonyl)-hexahydro-pyrrolo[3,2-b]pyrrole-1-carbonyl]-butyl}-4-piperazin-1-yl-benzamide;
1714. N-{3-methyl-1-[6-oxo-4-(pyridine-3-sulfonyl)-hexahydro-pyrrolo[3,2-b]pyrrole-1-carbonyl]-butyl}-4-piperazin-1-yl-benzamide;
1715. N-{3-methyl-1-[6-oxo-4-(1-oxy-pyridine-3-sulfonyl)-hexahydro-pyrrolo[3,2-b]pyrrole-1-carbonyl]-butyl}-4-piperazin-1-yl-benzamide;
1716. N-{3-methyl-1-[6-oxo-4-(pyridine sulfonyl)-hexahydro-pyrrolo[3,2-b]pyrrole-1-carbonyl]-butyl}-4-piperazin-1-yl-benzamide;
1717. N-{3-methyl-1-[6-oxo-4-(1-oxy-pyridinesulfonyl)-hexahydro-pyrrolo[3,2-b]pyrrole-1-carbonyl]-butyl}-4-piperazin-1-yl-benzamide;
1718. N-[3-methyl-1-(6-oxo-4-phenylacetyl-hexahydro-pyrrolo[3,2-b]pyrrole-1-carbonyl)-butyl]-4-piperazin-1-yl-benzamide;
1719. 4-[2-(4-piperazin-1-yl-benzoylamino)-4-methyl-pentanoyl]-3-oxo-hexahydro-pyrrolo[3,2-b]pyrrole-1-carboxylic acid phenylamide;
1720. 4-[2-(4-piperazin-1-yl-benzoylamino)-4-methyl-pentanoyl]-3-oxo hexahydro-pyrrolo[3,2-b]pyrrole-1-carboxylic acid isobutyl-amide;
1721. 4-[2-(4-piperazin-1-yl-benzoylamino)-4-methyl-pentanoyl]-3-oxo-hexahydro-pyrrolo[3,2-b]pyrrole-1-carboxylic acid phenyl ester,
1722. 4-[2-(4-piperazin-1-yl-benzoylamino)-4-methyl-pentanoyl]-3-oxo-hexahydro-pyrrolo[3,2-b]pyrrole-1-carboxylic acid isobutyl ester;
1723. N-{3-methyl-1-[6-oxo-4-(pyrrolidine-1-carbonyl)-hexahydro-pyrrolo[3,2-b]pyrrole-1-carbonyl]-butyl}-4-piperazin-1-yl-benzamide;
1724. N-{3-methyl-1-[6-oxo-4-(piperidine-1-carbonyl)-hexahydro-pyrrolo[3,2-b]pyrrole-1-carbonyl]-butyl}-4-piperazin-1-yl-benzamide;
1725. N-{3-methyl-1-[4-morpholinecarbonyl)-6-oxo-hexahydro-pyrrolo[3,2-b]pyrrole-1-carbonyl]-butyl}-4-piperazin-1-yl-benzamide;
1726. N-{3-methyl-1-[6-oxo-4-(piperazine-1-carbonyl)-hexahydro-pyrrolo[3,2-b]pyrrole-1-carbonyl]-butyl}-4-piperazin-1-yl-benzamide;
1727. N-{3-methyl-1-[4-(4-methyl-piperazine-1-carbonyl)-6-oxo-hexahydro-pyrrolo[3,2-b]pyrrole-1-carbonyl]-butyl}-4-piperazin-1-yl-benzamide;
1728. N-(3-methyl-1-{4-[2-(4-methyl-piperazin-1-yl)-acetyl]-6-oxo-hexahydro-pyrrolo[3,2-b]pyrrole-1-carbonyl}-butyl)-4-piperazin-1-yl-benzamide;
1729. N-{3-methyl-1-[6-oxo-4-(2-piperazin-1-yl-acetyl)-hexahydro-pyrrolo[3,2-b]pyrrole-1-carbonyl]-butyl}-4-piperazin-1-yl-benzamide;
1730. N-{1-[4-(furan-2-carbonyl)-6-oxo-hexahydro-pyrrolo[3,2-b]pyrrole-1-carbonyl]-3-methyl-butyl}-4-piperazin-1-yl-benzamide;
1731. N-{1-[4-(5-chloro-furan-2-carbonyl)-6-oxo-hexahydro-pyrrolo[3,2-b]pyrrole-1-carbonyl]-3-methyl-butyl}-4-piperazin-1-yl-benzamide;
1732. N-{1-[4-(thiophene-2-carbonyl)-6-oxo-hexahydro-pyrrolo[3,2-b]pyrrole-1-carbonyl]-3-methyl-butyl}-4-piperazin-1-yl-benzamide;
1733. N-{1-[4-(5-chloro-thiophene-2-carbonyl)-6-oxo-hexahydro-pyrrolo[3,2-b]pyrrole-1-carbonyl]-3-methyl-butyl}-4-piperazin-1-yl-benzamide;
1734. N-{1-[4-(furan-3-carbonyl)-6-oxo-hexahydro-pyrrolo[3,2-b]pyrrole-1-carbonyl]-3-methyl-butyl}-4-piperazin-1-yl-benzamide;
1735. N-{1-[4-(thiophene-3-carbonyl)-6-oxo-hexahydro-pyrrolo[3,2-b]pyrrole-1-carbonyl]-3-methyl-butyl}-4-piperazin-1-yl-benzamide;
1736. N-[1-(4-cyclopentanecarbonyl-6-oxo-hexahydro-pyrrolo[3,2-b]pyrrole-1-carbonyl)-3-methyl-butyl]-4-piperazin-1-yl-benzamide;
1737. N-[1-(4-Cyclohexanecarbonyl-6-oxo-hexahydro-pyrrolo[3,2-b]pyrrole-1-carbonyl)-3-methyl-butyl]-4-piperazin-1-yl-benzamide;
1738. N-{1-[4-(cyclopent-3-enecarbonyl)-6-oxo-hexahydro-pyrrolo[3,2-b]pyrrole-1-carbonyl]-3-methyl-butyl}-4-piperazin-1-yl-benzamide;
1739. N-{1-[4-(2-cyclopentyl-acetyl)-6-oxo-hexahydro-pyrrolo[3,2-b]pyrrole-1-carbonyl]-3-methyl-butyl}-4-piperazin-1-yl-benzamide;
1740. N-{1-[4-(2-cyclohexyl-acetyl)-6-oxo-hexahydro-pyrrolo[3,2-b]pyrrole-1-carbonyl]-3-methyl-butyl}-4-piperazin-1-yl-benzamide;
1741. 4-piperazin-1-yl-N-{3-methyl-1-[6-oxo-4-(pyrrolidine-2-carbonyl)-hexahydro-pyrrolo[3,2-b]pyrrole-1-carbonyl]-butyl}-benzamide;
1742. N-{1-[4-(1-Acetyl-pyrrolidine-2-carbonyl)-6-oxo-hexahydro-pyrrolo[3,2-b]pyrrole-1-carbonyl]-3-methyl-butyl}-4-piperazin-1-yl-benzamide;
1743. N-{3-methyl-1-[6-oxo piperidine-2-carbonyl)-hexahydro-pyrrolo[3,2-b]pyrrole-1-carbonyl]-butyl}-4-piperazin-1-yl-benzamide;
1744. N-{1-[4-(1-Acetyl-piperidine-2-carbonyl)-6-oxo-hexahydro-pyrrolo[3,2-b]pyrrole-1-carbonyl]-3-methyl-butyl}-4-piperazin-1-yl-benzamide;
1745. N-{3-methyl-1-[6-oxo-4-(tetrahydro-furan-2-carbonyl)-hexahydro-pyrrolo[3,2-b]pyrrole-1-carbonyl]-butyl}-4-piperazin-1-yl-benzamide;
1746. N-{1-[4-(1-Amino-cyclobutanecarbonyl)-6-oxo-hexahydro-pyrrolo[3,2-b]pyrrole-1-carbonyl]-3-methyl-butyl}-4-piperazin-1-yl-benzamide;
1747. N-{1-[4-(1-Amino-cyclopentanecarbonyl)-6-oxo-hexahydro-pyrrolo[3,2-b]pyrrole-1-carbonyl]-3-methyl-butyl}-4-piperazin-1-yl-benzamide;
1748. N-{1-[4-(1-Amino-cyclohexanecarbonyl)-6-oxo-hexahydro-pyrrolo[3,2-b]pyrrole-1-carbonyl]-3-methyl-butyl}-4-piperazin-1-yl-benzamide;
1749. N-{1-[4-(1-Acetylamino-cyclobutanecarbonyl)-6-oxo-hexahydro-pyrrolo[3,2-b]pyrrole-1-carbonyl]-3-methyl-butyl}-4-piperazin-1-yl-benzamide;
1750. N-{1-[4-(1-Acetylamino-cyclopentanecarbonyl)-6-oxo-hexahydro-pyrrolo[3,2-b]pyrrole-1-carbonyl]-3-methyl-butyl}-4-piperazin-1-yl-benzamide;
1751. N-{1-[4-(1-Acetylamino-cyclohexanecarbonyl)-6-oxo-hexahydro-pyrrolo[3,2-b]pyrrole-1-carbonyl]-3-methyl-butyl}-4-piperazin-1-yl-benzamide;
1752. N-{1-[4-(1-hydroxy-cyclobutanecarbonyl)-6-oxo-hexahydro-pyrrolo[3,2-b]pyrrole-1-carbonyl]-3-methyl-butyl}-4-piperazin-1-yl-benzamide;
1753. N-{1-[4-(1-hydroxy-cyclopentanecarbonyl)-6-oxo-hexahydro-pyrrolo[3,2-b]pyrrole-1-carbonyl]-3-methyl-butyl}-4-piperazin-1-yl-benzamide;
1754. N-{1-[4-(1-hydroxy-cyclohexanecarbonyl)-6-oxo-hexahydro-pyrrolo[3,2-b]pyrrole-1-carbonyl]-3-methyl-butyl}-4-piperazin-1-yl-benzamide;
1755. N-{1-[4-(1-methoxy-cyclobutanecarbonyl)-6-oxo-hexahydro-pyrrolo[3,2-b]pyrrole-1-carbonyl]-3-methyl-butyl}-4-piperazin-1-yl-benzamide;
1756. N-{1-[4-(1-methoxy-cyclopentanecarbonyl)-6-oxo-hexahydro-pyrrolo[3,2-b]pyrrole-1-carbonyl]-3-methyl-butyl}-4-piperazin-1-yl-benzamide;
1757. N-{1-[4-(1-methoxy-cyclohexanecarbonyl)-6-oxo-hexahydro-pyrrolo[3,2-b]pyrrole-1-carbonyl]-3-methyl-butyl}-4-piperazin-1-yl-benzamide;
1758. N-{1-[4-(2-Amino-2-cyclopentyl-acetyl)-6-oxo-hexahydro-pyrrolo[3,2-b]pyrrole-1-carbonyl]-3-methyl-butyl}-4-piperazin-1-yl-benzamide;
1759. N-{1-[4-(2-Amino-2-cyclohexyl-acetyl)-6-oxo-hexahydro-pyrrolo[3,2-b]pyrrole-1-carbonyl]-3-methyl-butyl}-4-piperazin-1-yl-benzamide;
1760. N-{1-[4-(2-Acetylamino-2-cyclopentyl-acetyl)-6-oxo-hexahydro-pyrrolo[3,2-b]pyrrole-1-carbonyl]-3-methyl-butyl}-4-piperazin-1-yl-benzamide;
1761. N-{1-[4-(2-Acetylamino-2-cyclohexyl-acetyl)-6-oxo-hexahydro-pyrrolo[3,2-b]pyrrole-1-carbonyl]-3-methyl-butyl}-4-piperazin-1-yl-benzamide;
1762. N-{1-[4-(2-Acetylamino-propionyl)-6-oxo-hexahydro-pyrrolo[3,2-b]pyrrole-1-carbonyl]-3-methyl-butyl}-4-piperazin-1-yl-benzamide;
1763. N-{1-[4-(2-Acetylamino-acetyl)-6-oxo-hexahydro-pyrrolo[3,2-b]pyrrole-1-carbonyl]-3-methyl-butyl}-4-piperazin-1-yl-benzamide;
1764. N-(1-{4-[2-(Acetyl-methyl-amino)-acetyl]-6-oxo-hexahydro-pyrrolo[3,2-b]pyrrole-1-carbonyl}-3-methyl-butyl)-4-piperazin-1-yl-benzamide;
1765. N-{1-[4-(2-dimethylamino-acetyl)-6-oxo-hexahydro-pyrrolo[3,2-b]pyrrole-1-carbonyl]-3-methyl-butyl}-4-piperazin-1-yl-benzamide;
1766. N-{1-[4-(2-Amino-butyryl)-6-oxo-hexahydro-pyrrolo[3,2-b]pyrrole-1-carbonyl]-3-methyl-butyl}-4-piperazin-1-yl-benzamide;
1767. N-{1-[4-(2-Acetylamino-butyryl)-6-oxo-hexahydro-pyrrolo[3,2-b]pyrrole-1-carbonyl]-3-methyl-butyl}-4-piperazin-1-yl-benzamide;
1768. N-{1-[4-(2-amino-3-methyl-butyryl)-6-oxo-hexahydro-pyrrolo[3,2-b]pyrrole-1-carbonyl]-3-methyl-butyl}-4-piperazin-1-yl-benzamide;
1769. N-{1-[4-(2-Acetylamino-3-methyl-butyryl)-6-oxo-hexahydro-pyrrolo[3,2-b]pyrrole-1-carbonyl]-3-methyl-butyl}-4-piperazin-1-yl-benzamide;
1770. N-{1-[4-(2-amino-3,3-dimethyl-butyryl)-6-oxo-hexahydro-pyrrolo[3,2-b]pyrrole-1-carbonyl]-3-methyl-butyl}-4-piperazin-1-yl-benzamide;
1771. N-{1-[4-(2-Acetylamino-3,3-dimethyl-butyryl)-6-oxo-hexahydro-pyrrolo[3,2-b]pyrrole-1-carbonyl]-3-methyl-butyl}-4-piperazin-1-yl-benzamide;
1772. N-{1-[4-(2-Amino-3-methyl-pentanoyl)-6-oxo-hexahydro-pyrrolo[3,2-b]pyrrole-1-carbonyl]-3-methyl-butyl}-4-piperazin-1-yl-benzamide;
1773. N-{1-[4-(2-Acetylamino-3-methyl-pentanoyl)-6-oxo-hexahydro-pyrrolo[3,2-b]pyrrole-1-carbonyl]-3-methyl-butyl}-4-piperazin-1-yl-benzamide;
1774. N-[3-methyl-1-(6-oxo-4-pentanoyl-hexahydro-pyrrolo[3,2-b]pyrrole-1-carbonyl)-butyl]-4-piperazin-1-yl-benzamide;
1775. N-{3-methyl-1-[4-(3-methyl-pentanoyl)-6-oxo-hexahydro-pyrrolo[3,2-b]pyrrole-1-carbonyl]-butyl}-4-piperazin-1-yl-benzamide;
1776. N-{3-methyl-1-[4-(4-methyl-pentanoyl)-6-oxo-hexahydro-pyrrolo[3,2-b]pyrrole-1-carbonyl]-butyl}-4-piperazin-1-yl-benzamide;
1777. N-{1-[4-(2-Aminomethyl-pentanoyl)-6-oxo-hexahydro-pyrrolo[3,2-b]pyrrole-1-carbonyl]-3-methyl-butyl}-4-piperazin-1-yl-benzamide;
1778. N-{1-[4-(2-Acetylamino-4-methyl-pentanoyl)-6-oxo-hexahydro-pyrrolo[3,2-b]pyrrole-1-carbonyl]-3-methyl-butyl}-4-piperazin-1-yl-benzamide;
1779. N-{1-[4-(2-Amino-4,4-dimethyl-pentanoyl)-6-oxo-hexahydro-pyrrolo[3,2-b]pyrrole-1-carbonyl]-3-methyl-butyl}-4-piperazin-1-yl-benzamide;
1780. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-piperazin-1-yl-benzamide;
1781. N-{1-[4-(2-Amino-pentanoyl)-6-oxo-hexahydro-pyrrolo[3,2-b]pyrrole-1-carbonyl]-3-methyl-butyl}-4-piperazin-1-yl-benzamide;
1782. N-{1-[4-(2-Acetylamino-pentanoyl)-6-oxo-hexahydro-pyrrolo[3,2-b]pyrrole-1-carbonyl]-3-methyl-butyl}-4-piperazin-1-yl-benzamide;
1783. N-{1-[4-(2-Amino-5-methyl-hexanoyl)-6-oxo-hexahydro-pyrrolo[3,2-b]pyrrole-1-carbonyl]-3-methyl-butyl}-4-piperazin-1-yl-benzamide;
1784. N-{1-[4-(2-Acetylamino-5-methyl-hexanoyl)-6-oxo-hexahydro-pyrrolo[3,2-b]pyrrole-1-carbonyl]-3-methyl-butyl}-4-piperazin-1-yl-benzamide;
1785. N-{1-[4-(2-hydroxy-3-methyl-butyryl)-6-oxo-hexahydro-pyrrolo[3,2-b]pyrrole-1-carbonyl]-3-methyl-butyl}-4-piperazin-1-yl-benzamide;
1786. N-{1-[4-(2-methoxy-3-methyl-butyryl)-6-oxo-hexahydro-pyrrolo[3,2-b]pyrrole-1-carbonyl]-3-methyl-butyl}-4-piperazin-1-yl-benzamide;
1787. N-{1-[4-(2-hydroxy-3,3-dimethyl-butyryl)-6-oxo-hexahydro-pyrrolo[3,2-b]pyrrole-1-carbonyl]-3-methyl-butyl}-4-piperazin-1-yl-benzamide;
1788. N-{1-[4-(2-methoxy-3,3-dimethyl-butyryl)-6-oxo-hexahydro-pyrrolo[3,2-b]pyrrole-1-carbonyl]-3-methyl-butyl}-4-piperazin-1-yl-benzamide;
1789. N-{1-[4-(2-hydroxy-3-methyl-pentanoyl)-6-oxo-hexahydro-pyrrolo[3,2-b]pyrrole-1-carbonyl]-3-methyl-butyl}-4-piperazin-1-yl-benzamide;
1790. N-{1-[4-(2-methoxy-3-methyl-pentanoyl)-6-oxo-hexahydro-pyrrolo[3,2-b]pyrrole-1-carbonyl]-3-methyl-butyl}-4-piperazin-1-yl-benzamide;
1791. N-{1-[4-(2-hydroxy-4-methyl-pentanoyl)-6-oxo-hexahydro-pyrrolo[3,2-b]pyrrole-1-carbonyl]-3-methyl-butyl}-4-piperazin-1-yl-benzamide;
1792. N-{1-[4-(2-methoxymethyl-pentanoyl)-6-oxo-hexahydro-pyrrolo[3,2-b]pyrrole-1-carbonyl]-3-methyl-butyl}-4-piperazin-1-yl-benzamide;
1793. N-{1-[4-(2-hydroxy-4,4-dimethyl-pentanoyl)-6-oxo-hexahydro-pyrrolo[3,2-b]pyrrole-1-carbonyl]-3-methyl-butyl}-4-piperazin-1-yl-benzamide;
1794. N-{1-[4-(2-methoxy-4,4-dimethyl-pentanoyl)-6-oxo-hexahydro-pyrrolo[3,2-b]pyrrole-1-carbonyl]-3-methyl-butyl}-4-piperazin-1-yl-benzamide;
1795. N-{1-[4-(2-hydroxy-pentanoyl)-6-oxo-hexahydro-pyrrolo[3,2-b]pyrrole-1-carbonyl]-3-methyl-butyl}-4-piperazin-1-yl-benzamide;
1796. N-{1-[4-(2-methoxy-pentanoyl)-6-oxo-hexahydro-pyrrolo[3,2-b]pyrrole-1-carbonyl]-3-methyl-butyl}-4-piperazin-1-yl-benzamide;
1797. N-{1-[4-(2-hydroxy-5-methyl-hexanoyl)-6-oxo-hexahydro-pyrrolo[3,2-b]pyrrole-1-carbonyl]-3-methyl-butyl}-4-piperazin-1-yl-benzamide;
1798. N-{1-[4-(2-methoxy-5-methyl-hexanoyl)-6-oxo-hexahydro-pyrrolo[3,2-b]pyrrole-1-carbonyl]-3-methyl-butyl}-4-piperazin-1-yl-benzamide;
1799. N-[1-(4-Benzoyl-6-oxo-hexahydro-pyrrolo[3,2-b]pyrrole-1-carbonyl)-3-methyl-butyl]-4-(4-methyl-piperazin-1-yl)benzamide;
1800. N-{3-methyl-1-[6-oxo-4-(1-oxy-pyridine-2-carbonyl)-hexahydro-pyrrolo[3,2-b]pyrrole-1-carbonyl]-butyl}-4-(4-methyl-piperazin-1-yl)-benzamide;
1801. N-{3-methyl-1-[6-oxo-4-(pyridine-3-carbonyl)-hexahydro-pyrrolo[3,2-b]pyrrole-1-carbonyl]-butyl}-4-(4-methyl-piperazin-1-yl)-benzamide;
1802. N-{3-methyl-1-[6-oxo-4-(1-oxy-pyridine-3-carbonyl)-hexahydro-pyrrolo[3,2-b]pyrrole-1-carbonyl]-butyl}-4-(4-methyl-piperazin-1-yl)-benzamide;
1803. N-{3-methyl-1-[6-oxo-4-(pyridine-4-carbonyl)-hexahydro-pyrrolo[3,2-b]pyrrole-1-carbonyl]-butyl}-4-(4-methyl-piperazin-1-yl)-benzamide;
1804. N-{3-methyl-1-[6-oxo-4-(1-oxy-pyridine-4-carbonyl)-hexahydro-pyrrolo[3,2-b]pyrrole-1-carbonyl]-butyl}-4-(4-methyl-piperazin-1-yl)-benzamide;
1805. N-[1-(4-Benzenesulfonyl-6-oxo-hexahydro-pyrrolo[3,2-b]pyrrole-1-carbonyl)-3-methyl-butyl]-4-(4-methyl-piperazin-1-yl)-benzamide;
1806. N-{3-methyl-1-[6-oxo-4-(pyridinesulfonyl)-hexahydro-pyrrolo[3,2-b]pyrrole-1-carbonyl]-butyl}-4-(4-methyl-piperazin-1-yl)-benzamide;
1807. N-{3-methyl-1-[6-oxo-4-(1-oxy-pyridine-4-sulfonyl)-hexahydro-pyrrolo[3,2-b]pyrrole-1-carbonyl]-butyl}-4-(4-methyl-piperazin-1-yl)-benzamide;
1808. N-[3-methyl-1-(6-oxo-4-phenylacetyl-hexahydro-pyrrolo[3,2-b]pyrrole-1-carbonyl)-butyl]-4-(4-methyl-piperazin-1-yl)-benzamide;
1809. 4-[2-(4-(4-methyl-piperazin-1-yl)-benzoylamino)-4-methyl-pentanoyl]-3-oxo-hexahydro-pyrrolo[3,2-b]pyrrole-1-carboxylic acid phenylamide;
1810. 4-[2-(4-(4-methyl-piperazin-1-yl)benzoylamino)-4-methyl-pentanoyl]-3-oxo-hexahydro-pyrrolo[3,2-b]pyrrole-1-carboxylic acid isobutyl-amide;
1811. 4-[2-(4-(4-methyl-piperazin-1-yl)-benzoylamino)-4-methyl-pentanoyl]-3-oxo-hexahydro-pyrrolo[3,2-b]pyrrole-1-carboxylic acid phenyl ester,
1812. 4-[2-(4-(4-methyl-piperazin-1-yl)-benzoylamino)-4-methyl-pentanoyl]-3-oxo-hexahydro-pyrrolo[3,2-b]pyrrole-1-carboxylic acid isobutyl ester,
1813. N-{3-methyl-1-[6-oxo-4-pyrrolidine-1-carbonyl)-hexahydro-pyrrolo[3,2-b]pyrrole-1-carbonyl]-butyl}-4-(4-methyl-piperazin-1-yl)-benzamide;
1814. N-{3-methyl-1-[6-oxo-4-(piperidine-1-carbonyl)-hexahydro-pyrrolo[3,2-b]pyrrole-1-carbonyl]-butyl-}-4-(4-methyl-piperazin-1-yl)-benzamide;
1815. N-{3-methyl-1-[4-(morpholine-4-carbonyl)-6-oxo-hexahydro-pyrrolo[3,2-b]pyrrole-1-carbonyl]-butyl}-4-(4-methyl-piperazin-1-yl)-benzamide;
1816. N-{3-methyl-1-[6-oxo-4-(piperazine-1-carbonyl)-hexahydro-pyrrolo[3,2-b]pyrrole-1-carbonyl]-butyl}-4-(4-methyl-piperazin-1-yl)-benzamide;
1817. N-{3-methyl-1-[4-(4-methyl-piperazine-1-carbonyl)-6-oxo-hexahydro-pyrrolo[3,2-b]pyrrole-1-carbonyl]-butyl}-4-(4-methyl-piperazin-1-yl)-benzamide;
1818. N-(3-methyl-1-{4-[2-(4-methyl-piperazin-1-yl)-acetyl]-6-oxo-hexahydro-pyrrolo[3,2-b]pyrrole-1-carbonyl}-butyl)-4-(4-methyl-piperazin-1-yl)-benzamide;
1819. N-{3-methyl-1-[6-oxo-4-(2-piperazin-1-yl-acetyl)-hexahydro-pyrrolo[3,2-b]pyrrole-1-carbonyl]-butyl}-4-(4-methyl-piperazin-1-yl)-benzamide;
1820. N-{1-[4-(furan-2-carbonyl)-6-oxo-hexahydro-pyrrolo[3,2-b]pyrrole-1-carbonyl]-3-methyl-butyl}-4-(4-methyl-piperazin-1-yl)-benzamide;
1821. N-{1-[4-(5-chloro-furan-2-carbonyl)-6-oxo-hexahydro-pyrrolo[3,2-b]pyrrole-1-carbonyl]-3-methyl-butyl}-4-(4-methyl-piperazin-1-yl)-benzamide;
1822. N-{1-[4-(thiophene-2-carbonyl)-6-oxo-hexahydro-pyrrolo[3,2-b]pyrrole-1-carbonyl]-3-methyl-butyl}-4-(4-methyl-piperazin-1-yl)-benzamide;
1823. N-{1-[4-(5-chloro-thiophene-2-carbonyl)-6-oxo-hexahydro-pyrrolo[3,2-b]pyrrole-1-carbonyl]-3-methyl-butyl}-4-(4-methyl-piperazin-1-yl)-benzamide;
1824. N-{1-[4-(furan-3-carbonyl)-6-oxo-hexahydro-pyrrolo[3,2-b]pyrrole-1-carbonyl]-3-methyl-butyl}-4-(4-methyl-piperazin-1-yl)-benzamide;
1825. N-{1-[4-(thiophene-3-carbonyl)-6-oxo-hexahydro-pyrrolo[3,2-b]pyrrole-1-carbonyl]-3-methyl-butyl}-4-(4-methyl-piperazin-1-yl)-benzamide;
1826. N-[1-(4-cyclopentanecarbonyl-6-oxo-hexahydro-pyrrolo[3,2-b]pyrrole-1-carbonyl)-3-methyl-butyl]-4-(4-methyl-piperazin-1-yl)-benzamide;
1827. N-[1-(4-cyclohexanecarbonyl-6-oxo-hexahydro-pyrrolo[3,2-b]pyrrole-1-carbonyl)-3-methyl-butyl]-4-(4-methyl-piperazin-1-yl)-benzamide;
1828. N-{1-[4-(cyclopent-3-enecarbonyl)-6-oxo-hexahydro-pyrrolo[3,2-b]pyrrole-1-carbonyl]-3-methyl-butyl}-4-(4-methyl-piperazin-1-yl)-benzamide;
1829. N-{1-[4-(2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
1830. N-{1-[4-(2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
1831. 4-(4-methyl-piperazin-1-yl)-N-{3-methyl-1-[6-oxo-4-(pyrrolidine-2-carbonyl)-hexahydro-pyrrolo[3,2-b]pyrrole-1-carbonyl]-butyl}-benzamide;
1832. N-{1-[4-(1-Acetyl-pyrrolidine-2-carbonyl)-6-oxo-hexahydro-pyrrolo[3,2-b]pyrrole-1-carbonyl]-3-methyl-butyl}-4-(4-methyl-piperazin-1-yl)-benzamide;
1833. N-{3-methyl-1-[6-oxo-4-(piperidine-2-carbonyl)-hexahydro-pyrrolo[3,2-b]pyrrole-1-carbonyl]-butyl}-4-(4-methyl-piperazin-1-yl)-benzamide;
1834. N-{1-[4-(1-Acetyl-piperidine-2-carbonyl)-6-oxo-hexahydro-pyrrolo[3,2-b]pyrrole-1-carbonyl]-3-methyl-butyl}-4-(4-methyl-piperazin-1-yl)-benzamide;
1835. N-{3-methyl-1-[6-oxo-4-(tetrahydro-furan-2-carbonyl)-hexahydro-pyrrolo[3,2-b]pyrrole-1-carbonyl]-butyl}-4-(4-methyl-piperazin-1-yl)-benzamide;
1836. N-{1-[4-(1-Amino-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
1837. N-{1-[4-(1-Amino-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
1838. N-{1-[4-(1-Amino-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
1839. N-{1-[4-(1-Acetylamino-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
1840. N-{1-[4-(1-Acetylamino-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
1841. N-{1-[4-(1-Acetylamino-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
1842. N-{1-[4-(1-hydroxy-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
1843. N-{1-[4-(1-hydroxy-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
1844. N-{1-[4-(1-hydroxy-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
1845. N-{1-[4-(1-methoxy-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
1846. N-{1-[4-(1-methoxy-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
1847. N-{1-[4-(1-methoxy-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
1848. N-{1-[4-(2-Amino-2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
1849. N-{1-[4-(2-Amino-2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
1850. N-{1-[4-(2-Acetylamino-2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
1851. N-{1-[4-(2-Acetylamino-2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
1852. N-{1-[4-(2-Acetylamino-propionyl)-6-oxo-hexahydro-pyrrolo[3,2-b]pyrrole-1-carbonyl]-3-methyl-butyl}-4-(4-methyl-piperazin-1-yl)-benzamide;
1853. N-{1-[4-(2-Acetylamino-acetyl)-6-oxo-hexahydro-pyrrolo[3,2-b]pyrrole-1-carbonyl]-3-methyl-butyl}-4-(4-methyl-piperazin-1-yl)-benzamide;
1854. N-(1-{4-[2-(Acetyl-methyl-amino)-acetyl]-6-oxo-hexahydro-pyrrolo[3,2-b]pyrrole-1-carbonyl}-3-methyl-butyl)-4-(4-methyl-piperazin-1-yl)-benzamide;
1855. N-{1-[4-(2-dimethylamino-acetyl)-6-oxo-hexahydro-pyrrolo[3,2-b]pyrrole-1-carbonyl]-3-methyl-butyl}-4-(4-methyl-piperazin-1-yl)-benzamide;
1856. N-{1-[4-(2-Amino-butyryl)-6-oxo-hexahydro-pyrrolo[3,2-b]pyrrole-1-carbonyl]-3-methyl-butyl}-4-(4-methyl-piperazin-1-yl)-benzamide;
1857. N-{1-[4-(2-Acetylamino-butyryl)-6-oxo-hexahydro-pyrrolo[3,2-b]pyrrole-1-carbonyl]-3-methyl-butyl}-4-(4-methyl-piperazin-1-yl)-benzamide;
1858. N-{1-[4-(2-amino-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
1859. N-{1-[4-(2-Acetylamino-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
1860. N-{1-[4-(2-amino-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
1861. N-{1-[4-(2-Acetylamino-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
1862. N-{1-[4-(2-Amino-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1863. N-{1-[4-(2-Acetylamino-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1864. N-[3-methyl-1-(6-oxo-4-pentanoyl-hexahydro-pyrrolo[3,2-b]pyrrole-1-carbonyl)-butyl]-4-(4-methyl-piperazin-1-yl)-benzamide;
1865. N-{3-methyl-1-[4-(3-methyl-pentanoyl)-6-oxo-hexahydro-pyrrolo[3,2-b]pyrrole-1-carbonyl]-butyl}-4-(4-methyl-piperazin-1-yl)-benzamide;
1866. N-{3-methyl-1-[4-(4-methyl-pentanoyl)-6-oxo-hexahydro-pyrrolo[3,2-b]pyrrole-1-carbonyl]-butyl}-4-(4-methyl-piperazin-1-yl)-benzamide;
1867. N-{1-[4-(2-Aminomethyl-pentanoyl)-6-oxo-hexahydro-pyrrolo[3,2-b]pyrrole-1-carbonyl]-3-methyl-butyl}-4-(4-methyl-piperazin-1-yl)-benzamide;
1868. N-{1-[4-(2-Acetylamino-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1869. N-{1-[4-(2-Amino-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
1870. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
1871. N-{1-[4-(2-Amino-pentanoyl)-6-oxo-hexahydro-pyrrolo[3,2-b]pyrrole-1-carbonyl]-3-methyl-butyl}-4-(4-methyl-piperazin-1-yl)-benzamide;
1872. N-{1-[4-(2-Acetylamino-pentanoyl)-6-oxo-hexahydro-pyrrolo[3,2-b]pyrrole-1-carbonyl]-3-methyl-butyl}-4-(4-methyl-piperazin-1-yl)-benzamide;
1873. N-{1-[4-(2-Amino-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
1874. N-{1-[4-(2-Acetylamino-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
1875. N-{1-[4-(2-hydroxy-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
1876. N-{1-[4-(2-methoxy-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
1877. N-{1-[4-(2-hydroxy-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
1878. N-{1-[4-(2-methoxy-3,3-dimethyl-butyryl)-6-oxo-hexahydro-pyrrolo[3,2-b]pyrrole-1-carbonyl]-3-methyl-butyl}-4-(4-methyl-piperazine-1-yl)-benzamide;
1879. N-{1-[4-(2-hydroxy-3-methyl-pentanoyl)-6-oxo-hexahydro-pyrrolo[3,2-b]pyrrole-1-carbonyl]-3-methyl-butyl}-4-(4-methyl-piperazine-1-yl)-benzamide;
1880. N-{1-[4-(2-methoxy-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1881. N-{1-[4-(2-hydroxy methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1882. N-{1-[4-(2-methoxy-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
1883. N-{1-[4-(2-hydroxy-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
1884. N-{1-[4-(2-methoxy-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
1885. N-{1-[4-(2-hydroxy-pentanoyl)-6-oxo-hexahydro-pyrrolo[3,2-b]pyrrole-1-carbonyl]-3-methyl-butyl}-4-(4-methyl-piperazin-1-yl)-benzamide;
1886. N-{1-[4-(2-methoxy-pentanoyl)-6-oxo-hexahydro-pyrrolo[3,2-b]pyrrole-1-carbonyl]-3-methyl-butyl}-4-(4-methyl-piperazin-1-yl)-benzamide;
1887. N-{1-[4-(2-hydroxy-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
1888. N-{1-[4-(2-methoxy-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
1889. N-[1-(4-Benzoyl-6-oxo-hexahydro-pyrrolo[3,2-b]pyrrole-1-carbonyl)-3-methyl-butyl]-4-aminobenzamide;
1890. 4-Amino-N-{3-methyl-1-[6-oxo-4-(pyridine-2-carbonyl)-hexahydro-pyrrolo[3,2-b]pyrrole-1-carbonyl]-butyl}-benzamide;
1891. 4-Amino-N-{3-methyl-1-[6-oxo-4-(1-oxy-pyridine-2-carbonyl)-hexahydro-pyrrolo[3,2-b]pyrrole-1-carbonyl]-butyl}-benzamide;
1892. 4-Amino-N-{3-methyl-1-[6-oxo-4-(pyridine-3-carbonyl)-hexahydro-pyrrolo[3,2-b]pyrrole-1-carbonyl]-butyl}-benzamide;
1893. 4-Amino-N-{3-methyl-1-[6-oxo-4-(1-oxy-pyridine-3-carbonyl)-hexahydro-pyrrolo[3,2-b]pyrrole-1-carbonyl]-butyl}-benzamide;
1894. 4-Amino-N-{3-methyl-1-[6-oxo-4-(pyridine-4-carbonyl)-hexahydro-pyrrolo[3,2-b]pyrrole-1-carbonyl]-butyl}-benzamide;
1895. 4-Amino-N-{3-methyl-1-[6-oxo-4-(1-oxy-pyridine-4-carbonyl)-hexahydro-pyrrolo[3,2-b]pyrrole-1-carbonyl]-butyl}-benzamide;
1896. N-[1-(4-Benzenesulfonyl-6-oxo-hexahydro-pyrrolo[3,2-b]pyrrole-1-carbonyl)-3-methyl-butyl]-4-amino-benzamide;
1897. 4-Amino-N-{3-methyl-1-[6-oxo-4-(pyridine-2-sulfonyl)-hexahydro-pyrrolo[3,2-b]pyrrole-1-carbonyl]-butyl}-benzamide;
1898. 4-Amino-N-{3-methyl-1-[6-oxo-4-(1-oxy-pyridine-2-sulfonyl)-hexahydro-pyrrolo[3,2-b]pyrrole-1-carbonyl]-butyl}-benzamide;
1899. 4-Amino-N-{3-methyl-1-[6-oxo-4-(pyridine-3-sulfonyl)-hexahydro-pyrrolo[3,2-b]pyrrole-1-carbonyl]-butyl}-benzamide;
1900. 4-Amino-N-{3-methyl-1-[6-oxo-4-(1-oxy-pyridine-3-sulfonyl)-hexahydro-pyrrolo[3,2-b]pyrrole-1-carbonyl]-butyl}-benzamide;
1901. 4-Amino-N-{3-methyl-1-[6-oxo-4-(pyridine-4-sulfonyl)-hexahydro-pyrrolo[3,2-b]pyrrole-1-carbonyl]-butyl}-benzamide;
1902. 4-Amino-N-{3-methyl-1-[6-oxo-4-(1-oxy-pyridinesulfonyl)-hexahydro-pyrrolo[3,2-b]pyrrole-1-carbonyl]-butyl}-benzamide;
1903. 4-Amino-N-[3-methyl-1-(6-oxo-4-phenylacetyl-hexahydro-pyrrolo[3,2-b]pyrrole-1-carbonyl)-butyl]-benzamide;
1904. 4-[2-(4-amino-benzoylamino)-4-methyl-pentanoyl]-3-oxo-hexahydro-pyrrolo[3,2-b]pyrrole-1-carboxylic acid phenylamide;
1905. 4-[2-(4-amino-benzoylamino)-4-methyl-pentanoyl]-3-oxo-hexahydro-pyrrolo[3,2-b]pyrrole-1-carboxylic acid isobutyl-amide;
1906. 4-[2-(4-amino-benzoylamino)-4-methyl-pentanoyl]-3-oxo-hexahydro-pyrrolo[3,2-b]pyrrole-1-carboxylic acid phenyl ester;
1907. 4-[2-(4-amino-benzoylamino)-4-methyl-pentanoyl]-3-oxo-hexahydro-pyrrolo[3,2-b]pyrrole-1-carboxylic acid isobutyl ester,
1908. 4-Amino-N-{3-methyl-1-[6-oxo-4-(pyrrolidine-1-carbonyl)-hexahydro-pyrrolo[3,2-b]pyrrole-1-carbonyl]-butyl}-benzamide;
1909. 4-Amino-N-{3-methyl-1-[6-oxo-4-(piperidine-1-carbonyl)-hexahydro-pyrrolo[3,2-b]pyrrole-1-carbonyl]-butyl}-benzamide;
1910. 4-Amino-N-{3-methyl-1-[4-(morpholin-carbonyl)-6-oxo-hexahydro-pyrrolo[3,2-b]pyrrole-1-carbonyl]-butyl}-benzamide;
1911. 4-Amino-N-{3-methyl-1-[6-oxo-4-(piperazine-1-carbonyl)hexahydro-pyrrolo[3,2-b]pyrrole-1-carbonyl]-butyl}-benzamide;
1912. 4-Amino-N-{3-methyl-1-[4-(4-methyl-piperazine-1-carbonyl)-6-oxo-hexahydro-pyrrolo[3,2-b]pyrrole-1-carbonyl]-butyl}-benzamide;
1913. 4-Amino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
1914. 4-Amino-N-{3-methyl-1-[6-oxo-4-(2-piperazin-1-yl-acetyl)-hexahydro-pyrrolo[3,2-b]pyrrole-1-carbonyl]-butyl}-benzamide;
1915. 4-Amino-N-{1-[4-(furan-2-carbonyl)-6-oxo-hexahydro-pyrrolo[3,2-b]pyrrole-1-carbonyl]-3-methyl-butyl}-benzamide;
1916. 4-Amino-N-{1-[4-(5-chloro-furan-2-carbonyl)-6-oxo-hexahydro-pyrrolo[3,2-b]pyrrole-1-carbonyl]-3-methyl-butyl}-benzamide;
1917. 4-Amino-N-{1-[4-(thiophene-2-carbonyl)-6-oxo-hexahydro-pyrrolo[3,2-b]pyrrole-1-carbonyl]-3-methyl-butyl}-benzamide;
1918. 4-Amino-N-{1-[4-(5-chloro-thiophene-2-carbonyl)-6-oxo-hexahydro-pyrrolo[3,2-b]pyrrole-1-carbonyl]-3-methyl-butyl}-benzamide;
1919. 4-Amino-N-{1-[4-(furan-3-carbonyl)-6-oxo-hexahydro-pyrrolo[3,2-b]pyrrole-1-carbonyl]-3-methyl-butyl}-benzamide;
1920. 4-Amino-N-{1-[4-(thiophene-3-carbonyl)-6-oxo-hexahydro-pyrrolo[3,2-b]pyrrole-1-carbonyl]-3-methyl-butyl}-benzamide;
1921. 4-Amino-N-[1-(4-cyclopentanecarbonyl-6-oxo-hexahydro-pyrrolo[3,2-b]pyrrole-1-carbonyl)-3-methyl-butyl]-benzamide;
1922. 4-Amino-N-[1-(4-cyclohexanecarbonyl-6-oxo-hexahydro-pyrrolo[3,2-b]pyrrole-1-carbonyl)-3-methyl-butyl]-benzamide;
1923. 4-Amino-N-{1-[4-(cyclopent-3-enecarbonyl)-6-oxo-hexahydro-pyrrolo[3,2-b]pyrrole-1-carbonyl]-3-methyl-butyl}-benzamide;
1924. 4-Amino-N-{1-[4-(2-cyclopentyl-acetyl)-6-oxo-hexahydro-pyrrolo[3,2-b]pyrrole-1-carbonyl]-3-methyl-butyl}-benzamide;
1925. 4-Amino-N-{1-[4-(2-cyclohexyl-acetyl)-6-oxo-hexahydro-pyrrolo[3,2-b]pyrrole-1-carbonyl]-3-methyl-butyl}-benzamide;
1926. 4-Amino-N-{3-methyl-1-[6-oxo-4-pyrrolidine-2-carbonyl)-hexahydro-pyrrolo[3,2-b]pyrrole-1-carbonyl]-butyl}-benzamide;
1927. N-{1-[4-(1-Acetyl-pyrrolidine-2-carbonyl)-6-oxo-hexahydro-pyrrolo[3,2-b]pyrrole-1-carbonyl]-3-methyl-butyl}-4-amino-benzamide;
1928. 4-Amino-N-{3-methyl-1-[6-oxo-4-(piperidine-2-carbonyl)-hexahydro-pyrrolo[3,2-b]pyrrole-1-carbonyl]-butyl}-benzamide;
1929. N-{1-[4-(1-Acetyl-piperidine-2-carbonyl)-6-oxo-hexahydro-pyrrolo[3,2-b]pyrrole-1-carbonyl]-3-methyl-butyl}-4-amino-benzamide;
1930. 4-Amino-N-{3-methyl-1-[6-oxo-4-(tetrahydro-furan-2-carbonyl)-hexahydro-pyrrolo[3,2-b]pyrrole-1-carbonyl]-butyl}-benzamide;
1931. N-{1-[4-(1-Amino-cyclobutanecarbonyl)-6-oxo-hexahydro-pyrrolo[3,2-b]pyrrole-1-carbonyl]-3-methyl-butyl}-4-Amino-benzamide;
1932. N-{1-[4-(1-Amino-cyclopentanecarbonyl)-6-oxo-hexahydro-pyrrolo[3,2-b]pyrrole-1-carbonyl]-3-methyl-butyl}-4-amino-benzamide;
1933. N-{1-[4-(1-Amino-cyclohexanecarbonyl)-6-oxo-hexahydro-pyrrolo[3,2-b]pyrrole-1-carbonyl]-3-methyl-butyl}-4-amino-benzamide;
1934. N {1-[4-(1-Acetylamino-cyclobutanecarbonyl)-6-oxo-hexahydro-pyrrolo[3,2-b]pyrrole-1-carbonyl]-3-methyl-butyl}-4-amino-benzamide;
1935. N-{1-[4-(1-Acetylamino-cyclopentanecarbonyl)-6-oxo-hexahydro-pyrrolo[3,2-b]pyrrole-1-carbonyl]-3-methyl-butyl}-4-amino-benzamide;
1936. N-{1-[4-(1-Acetylamino-cyclohexanecarbonyl)-6-oxo-hexahydro-pyrrolo[3,2-b]pyrrole-1-carbonyl]-3-methyl-butyl}-4-amino-benzamide;
1937. 4-Amino-N-{1-[4-(1-hydroxy-cyclobutanecarbonyl)-6-oxo-hexahydro-pyrrolo[3,2-b]pyrrole-1-carbonyl)-3-methyl-butyl}-benzamide;
1938. 4-Amino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
1939. 4-Amino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
1940. 4-Amino-N-{1-[4-(1-methoxy-cyclobutanecarbonyl)-6-oxo-hexahydro-pyrrolo[3,2-b]pyrrole-1-carbonyl]-3-methyl-butyl}-benzamide;
1941. 4-Amino-N-{1-[4-(1-methoxy-cyclopentanecarbonyl)-6-oxo-hexahydro-pyrrolo[3,2-b]pyrrole-1-carbonyl]-3-methyl-butyl}-benzamide;
1942. 4-Amino-N-{1-[4-(1-methoxy-cyclohexanecarbonyl)-6-oxo-hexahydro-pyrrolo[3,2-b]pyrrole-1-carbonyl]-3-methyl-butyl}-benzamide;
1943. N-{1-[4-(2-amino-2-cyclopentyl-acetyl)-6-oxo-hexahydro-pyrrolo[3,2-b]pyrrole-1-carbonyl]-3-methyl-butyl}-4-amino-benzamide;
1944. N-{1-[4-(2-amino-2-cyclohexyl-acetyl)-6-oxo-hexahydro-pyrrolo[3,2-b]pyrrole-1-carbonyl]-3-methyl-butyl}-4-amino-benzamide;
1945. N-{1-[4-(2-acetylamino-2-cyclopentyl-acetyl)-6-oxo-hexahydro-pyrrolo[3,2-b]pyrrole-1-carbonyl]-3-methyl-butyl}-4-amino-benzamide;
1946. N-{1-[4-(2-acetylamino-2-cyclohexyl-acetyl)-6-oxo-hexahydro-pyrrolo[3,2-b]pyrrole-1-carbonyl]-3-methyl-butyl}-4-amino-benzamide;
1947. N-{1-[4-(2-acetylamino-propionyl)-6-oxo-hexahydro-pyrrolo[3,2-b]pyrrole-1-carbonyl]-3-methyl-butyl}-4-amino-benzamide;
1948. N-{1-[4-(2-acetylamino-acetyl)-6-oxo-hexahydro-pyrrolo[3,2-b]pyrrole-1-carbonyl]-3-methyl-butyl}-4-amino-benzamide;
1949. N-(1-{4-[2-(acetyl-methyl-amino)-acetyl]-6-oxo-hexahydro-pyrrolo[3,2-b]pyrrole-1-carbonyl}-3-methyl-butyl)-4-amino-benzamide;
1950. 4-Amino-N-{1-[4-(2-dimethylamino-acetyl)-6-oxo-hexahydro-pyrrolo[3,2-b]pyrrole-1-carbonyl]-3-methyl-butyl}-benzamide;
1951. N-{1-[4-(2-amino-butyryl)-6-oxo-hexahydro-pyrrolo[3,2-b]pyrrole-1-carbonyl]-3-methyl-butyl}-4-amino-benzamide;
1952. N-{1-[4-(2-acetylamino-butyryl)-6-oxo-hexahydro-pyrrolo[3,2-b]pyrrole-1-carbonyl]-3-methyl-butyl}-4-amino-benzamide;
1953. N-{1-[4-(2-amino-3-methyl-butyryl)-6-oxo-hexahydro-pyrrolo[3,2-b]pyrrole-1-carbonyl]-3-methyl-butyl}-4-amino-benzamide;
1954. N-{1-[4-(2-acetylamino-3-methyl-butyryl)-6-oxo-hexahydro-pyrrolo[3,2-b]pyrrole-1-carbonyl]-3-methyl-butyl}-4-amino-benzamide;
1955. N-{1-[4-(2-amino-3,3-dimethyl-butyryl)-6-oxo-hexahydro-pyrrolo[3,2-b]pyrrole-1-carbonyl]-3-methyl-butyl}-4-amino-benzamide;
1956. N-{1-[4-(2-acetylamino-3,3-dimethyl-butyryl)-6-oxo-hexahydro-pyrrolo[3,2-b]pyrrole-1-carbonyl]-3-methyl-butyl}-4-amino-benzamide;
1957. N-{1-[4-(2-amino-3-methyl-pentanoyl)-6-oxo-hexahydro-pyrrolo[3,2-b]pyrrole-1-carbonyl]-3-methyl-butyl}-4-amino-benzamide;
1958. N-{1-[4-(2-acetylamino-3-methyl-pentanoyl)-6-oxo-hexahydro-pyrrolo[3,2-b]pyrrole-1-carbonyl]-3-methyl-butyl}-4-amino-benzamide;
1959. 4-Amino-N-[3-methyl-1-(6-oxo-4-pentanoyl-hexahydro-pyrrolo[3,2-b]pyrrole-1-carbonyl)-butyl]-benzamide;
1960. 4-Amino-N-{3-methyl-1-[4-(3-methyl-pentanoyl)-6-oxo-hexahydro-pyrrolo[3,2-b]pyrrole-1-carbonyl]-butyl}-benzamide;
1961. 4-Amino-N-{3-methyl-1-[4-(4-methyl-pentanoyl)-6-oxo-hexahydro-pyrrolo[3,2-b]pyrrole-1-carbonyl]-butyl}-benzamide;
1962. N-{1-[4-(2-amino-4-methyl-pentanoyl)-6-oxo-hexahydro-pyrrolo[3,2-b]pyrrole-1-carbonyl]-3-methyl-butyl}-4-amino-benzamide;
1963. N-{1-[4-(2-acetylamino-methyl-pentanoyl)-6-oxo-hexahydro-pyrrolo[3,2-b]pyrrole-1-carbonyl]-3-methyl-butyl}-4-amino-benzamide;
1964. N-{1-[4-(2-amino-4,4-dimethyl-pentanoyl)-6-oxo-hexahydro-pyrrolo[3,2-b]pyrrole-1-carbonyl]-3-methyl-butyl}-4-amino-benzamide;
1965. N-{1-[4-(2-acetylamino-4,4-dimethyl-pentanoyl)-6-oxo-hexahydro-pyrrolo[3,2-b]pyrrole-1-carbonyl]-3-methyl-butyl}-4-amino-benzamide;
1966. N-{1-[4-(2-amino-pentanoyl)-6-oxo-hexahydro-pyrrolo[3,2-b]pyrrole-1-carbonyl]-3-methyl-butyl}-4-amino-benzamide;
1967. N-{1-[4-(2-acetylamino-pentanoyl)-6-oxo-hexahydro-pyrrolo[3,2-b]pyrrole-1-carbonyl]-3-methyl-butyl}-4-amino-benzamide;
1968. N-{1-[4-(2-amino-5-methyl-hexanoyl)-6-oxo-hexahydro-pyrrolo[3,2-b]pyrrole-1-carbonyl]-3-methyl-butyl}-4-amino-benzamide;
1969. N-{1-[4-(2-acetylamino-5-methyl-hexanoyl)-6-oxo-hexahydro-pyrrolo[3,2-b]pyrrole-1-carbonyl]-3-methyl-butyl}-4-amino-benzamide;
1970. N-{1-[4-(2-hydroxy-3-methyl-butyryl)-6-oxo-hexahydro-pyrrolo[3,2-b]pyrrole-1-carbonyl]-3-methyl-butyl}-4-amino-benzamide;
1971. N-{1-[4-(2-methoxy-3-methyl-butyryl)-6-oxo-hexahydro-pyrrolo[3,2-b]pyrrole-1-carbonyl]-3-methyl-butyl}-4-amino-benzamide;
1972. 4-Amino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
1973. 4-Amino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
1974. 4-Amino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
1975. 4-Amino-N-{1-[4-(2-methoxy-3-methyl-pentanoyl)-6-oxo-hexahydro-pyrrolo[3,2-b]pyrrole-1-carbonyl]-3-methyl-butyl}-benzamide;
1976. 4-Amino-N-{1-[4-(2-hydroxymethyl-pentanoyl)-6-oxo-hexahydro-pyrrolo[3,2-b]pyrrole-1-carbonyl]-3-methyl-butyl}-benzamide;
1977. 4-Amino-N-{1-[4-(2-methoxy-methyl-pentanoyl)-6-oxo-hexahydro-pyrrolo[3,2-b]pyrrole-1-carbonyl]-3-methyl-butyl}-benzamide;
1978. 4-Amino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
1979. 4-Amino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
1980. 4-Amino-N-{1-[4-(2-hydroxy-pentanoyl)-6-oxo-hexahydro-pyrrolo[3,2-b]pyrrole-1-carbonyl]-3-methyl-butyl}-benzamide;
1981. 4-Amino-N-{1-[4-(2-methoxy-pentanoyl)-6-oxo-hexahydro-pyrrolo[3,2-b]pyrrole-1-carbonyl]-3-methyl-butyl}-benzamide;
1982. 4-Amino-N-{1-[4-(2-hydroxy-5-methyl-hexanoyl)-6-oxo-hexahydro-pyrrolo[3,2-b]pyrrole-1-carbonyl]-3-methyl-butyl}-benzamide;
1983. 4-Amino-N-{1-[4-(2-methoxy-5-methyl-hexanoyl)-6-oxo-hexahydro-pyrrolo[3,2-b]pyrrole-1-carbonyl]-3-methyl-butyl}-benzamide;
1984. N-[1-(4-Benzoyl-6-oxo-hexahydro-pyrrolo[3,2-b]pyrrole-1-carbonyl)-3-methyl-butyl]-4-diethylaminobenzamide;
1985. 4-Diethylamino-N-{3-methyl-1-[6-oxo-4-(pyridine-2-carbonyl)-hexahydro-pyrrolo[3,2-b]pyrrole-1-carbonyl]-butyl}-benzamide;
1986. 4-Diethylamino-N-{3-methyl-1-[6-oxo-4-(1-oxy-pyridine-2-carbonyl) hexahydro-pyrrolo[3,2-b]pyrrole-1-carbonyl]-butyl}-benzamide;
1987. 4-Diethylamino-N-{3-methyl-1-[6-oxo-4-(pyridine-3-carbonyl)-hexahydro-pyrrolo[3,2-b]pyrrole-1-carbonyl]-butyl}-benzamide;
1988. 4-Diethylamino-N-{3-methyl-1-[6-oxo-4-(1-oxy-pyridine-3-carbonyl)-hexahydro-pyrrolo[3,2-b]pyrrole-1-carbonyl]-butyl}-benzamide;
1989. 4-Diethylamino-N-{3-methyl-1-[6-oxo-4-(pyridine-4-carbonyl) hexahydro-pyrrolo[3,2-b]pyrrole-1-carbonyl]-butyl}-benzamide;
1990. 4-Diethylamino-N-{3-methyl-1-[6-oxo-4-(1-oxy-pyridine-4-carbonyl)-hexahydro-pyrrolo[3,2-b]pyrrole-1-carbonyl]-butyl}-benzamide;
1991. N-[1-(4-Benzenesulfonyl-6-oxo-hexahydro-pyrrolo[3,2-b]pyrrole-1-carbonyl)-3-methyl-butyl]-4-diethylamino-benzamide;
1992. 4-Diethylamino-N-{3-methyl-1-[6-oxo-4-(pyridine-2-sulfonyl)-hexahydro-pyrrolo[3,2-b]pyrrole-1-carbonyl]-butyl}-benzamide;
1993. 4-Diethylamino-N-{3-methyl-1-[6-oxo-4-(1-oxy-pyridine-2-sulfonyl)-hexahydro-pyrrolo[3,2-b]pyrrole-1-carbonyl]-butyl}-benzamide;
1994. 4-Diethylamino-N-{3-methyl-1-[6-oxo-4-(pyridine-3-sulfonyl)-hexahydro-pyrrolo[3,2-b]pyrrole-1-carbonyl]-butyl}-benzamide;
1995. 4-Diethylamino-N-{3-methyl-1-[6-oxo-4-(1-oxy-pyridine-3-sulfonyl)-hexahydro-pyrrolo[3,2-b]pyrrole-1-carbonyl]-butyl}-benzamide;
1996. 4-Diethylamino-N-{3-methyl-1-[6-oxo-4-(pyridine sulfonyl)-hexahydro-pyrrolo[3,2-b]pyrrole-1-carbonyl]-butyl}-benzamide;
1997. 4-Diethylamino-N-{3-methyl-1-[6-oxo-4-(1-oxy-pyridine-4-sulfonyl)-hexahydro-pyrrolo[3,2-b]pyrrole-1-carbonyl]-butyl}-benzamide;
1998. 4-Diethylamino-N-[3-methyl-1-(6-oxo-4-phenylacetyl-hexahydro-pyrrolo[3,2-b]pyrrole-1-carbonyl)-butyl]-benzamide;
1999. 4-[2-(4-diethylamino-benzoylamino)-4-methyl-pentanoyl]-3-oxo-hexahydro-pyrrolo[3,2-b]pyrrole-1-carboxylic acid phenylamide;
2000. 4-[2-(4-diethylamino-benzoylamino)-4-methyl-pentanoyl]-3-oxo-hexahydro-pyrrolo[3,2-b]pyrrole-1-carboxylic acid isobutyl-amide;
2001. 4-[2-(4-diethylamino-benzoylamino)-4-methyl-pentanoyl]-3-oxo-hexahydro-pyrrolo[3,2-b]pyrrole-1-carboxylic acid phenyl ester,
2002. 4-[2-(4-diethylamino-benzoylamino)-4-methyl-pentanoyl]-3-oxo-hexahydro-pyrrolo[3,2-b]pyrrole-1-carboxylic acid isobutyl ester,
2003. 4-Diethylamino-N-{3-methyl-1-[6-oxo-4-(pyrrolidine-1-carbonyl)-hexahydro-pyrrolo[3,2-b]pyrrole-1-carbonyl]-butyl}-benzamide;
2004. 4-Diethylamino-N-{3-methyl-1-[6-oxo-4-(piperidine-1-carbonyl)-hexahydro-pyrrolo[3,2-b]pyrrole-1-carbonyl]-butyl}-benzamide;
2005. 4-Diethylamino-N-{3-methyl-1-[4-(morpholinecarbonyl)-6-oxo-hexahydro-pyrrolo[3,2-b]pyrrole-1-carbonyl]-butyl}-benzamide;
2006. 4-Diethylamino-N-{3-methyl-1-[6-oxo-4-(piperazine-1-carbonyl)-hexahydro-pyrrolo[3,2-b]pyrrole-1-carbonyl]-butyl}-benzamide;
2007. 4Diethylamino-N-{3-methyl-1-[4-(4-methyl-piperazine-1-carbonyl)-6-oxo-hexahydro-pyrrolo[3,2-b]pyrrole-1-carbonyl]-butyl}-benzamide;
2008. 4-Diethylamino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
2009. 4-Diethylamino-N-{3-methyl-1-[6-oxo-4-(2-piperazin-1-yl-acetyl)-hexahydro-pyrrolo[3,2-b]pyrrole-1-carbonyl]-butyl}-benzamide;
2010. 4-Diethylamino-N-{1-[4-(furan-2-carbonyl)-6-oxo-hexahydro-pyrrolo[3,2-b]pyrrole-1-carbonyl]-3-methyl-butyl}-benzamide;
2011. 4-Diethylamino-N-{1-[4-(5-chloro-furan-2-carbonyl)-6-oxo-hexahydro-pyrrolo[3,2-b]pyrrole-1-carbonyl]-3-methyl-butyl}-benzamide;
2012. 4-Diethylamino-N-{1-[4-(thiophene-2-carbonyl)-6-oxo-hexahydro-pyrrolo[3,2-b]pyrrole-1-carbonyl]-3-methyl-butyl}-benzamide;
2013. 4-Diethylamino-N-{1-[4-(5-chloro-thiophene-2-carbonyl)-6-oxo-hexahydro-pyrrolo[3,2-b]pyrrole-1-carbonyl]-3-methyl-butyl}-benzamide;
2014. 4-Diethylamino-N-{1-[4-(furan-3-carbonyl)-6-oxo-hexahydro-pyrrolo[3,2-b]pyrrole-1-carbonyl]-3-methyl-butyl}-benzamide;
2015. 4-Diethylamino-N-{1-[4-(thiophene-3-carbonyl)-6-oxo-hexahydro-pyrrolo[3,2-b]pyrrole-1-carbonyl]-3-methyl-butyl}-benzamide;
2016. 4-Diethylamino-N-[1-(4-cyclopentanecarbonyl-6-oxo-hexahydro-pyrrolo[3,2-b]pyrrole-1-carbonyl)-3-methyl-butyl]-benzamide;
2017. 4-Diethylamino-N-[1-(4-cyclohexanecarbonyl-6-oxo-hexahydro-pyrrolo[3,2-b]pyrrole-1-carbonyl)-3-methyl-butyl]-benzamide;
2018. 4-Diethylamino-N-{1-[4-(cyclopent-3-enecarbonyl)-6-oxo-hexahydro-pyrrolo[3,2-b]pyrrole-1-carbonyl]-3-methyl-butyl}-benzamide;
2019. 4-Diethylamino-N-{1-[4-(2-cyclopentyl-acetyl)-6-oxo-hexahydro-pyrrolo[3,2-b]pyrrole-1-carbonyl]-3-methyl-butyl}-benzamide;
2020. 4-Diethylamino-N-{1-[4-(2-cyclohexyl-acetyl)-6-oxo-hexahydro-pyrrolo[3,2-b]pyrrole-1-carbonyl]-3-methyl-butyl}-benzamide;
2021. 4-Diethylamino-N-{3-methyl-1-[6-oxo-4-(pyrrolidine-2-carbonyl)-hexahydro-pyrrolo[3,2-b]pyrrole-1-carbonyl]-butyl}-benzamide;
2022. N-{1-[4-(1-Acetyl-pyrrolidine-2-carbonyl)-6-oxo-hexahydro-pyrrolo[3,2-b]pyrrole-1-carbonyl]-3-methyl-butyl}-4-diethylamino-benzamide;
2023. 4-Diethylamino-N-{3-methyl-1-[6-oxo-4-(piperidine-2-carbonyl)-hexahydro-pyrrolo[3,2-b]pyrrole-1-carbonyl]-butyl}-benzamide;
2024. N-{1-[4-(1-Acetyl-piperidine-2-carbonyl)-6-oxo-hexahydro-pyrrolo[3,2-b]pyrrole-1-carbonyl]-3-methyl-butyl}-4-diethylamino-benzamide;
2025. 4-Diethylamino-N-{3-methyl-1-[6-oxo-4-(tetrahydro-furan-2-carbonyl)-hexahydro-pyrrolo[3,2-b]pyrrole-1-carbonyl]-butyl}-benzamide;
2026. N-{1-[4-(1-Amino-cyclobutanecarbonyl)-6-oxo-hexahydro-pyrrolo[3,2-b]pyrrole-1-carbonyl]-3-methyl-butyl}-4-diethylamino-benzamide;
2027. N-{1-[4-(1-Amino-cyclopentanecarbonyl)-6-oxo-hexahydro-pyrrolo[3,2-b]pyrrole-1-carbonyl]-3-methyl-butyl}-4-diethylamino-benzamide;
2028. N-{1-[4-(1-Amino-cyclohexanecarbonyl)-6-oxo-hexahydro-pyrrolo[3,2-b]pyrrole-1-carbonyl]-3-methyl-butyl}-4-diethylamino-benzamide;
2029. N-{1-[4-(1-Acetylamino-cyclobutanecarbonyl)-6-oxo-hexahydro-pyrrolo[3,2-b]pyrrole-1-carbonyl]-3-methyl-butyl}-4-diethylamino-benzamide;
2030. N-{1-[4-(1-Acetylamino-cyclopentanecarbonyl)-6-oxo-hexahydro-pyrrolo[3,2-b]pyrrole-1-carbonyl]-3-methyl-butyl}-4-diethylamino-benzamide;
2031. N-{1-[4-(1-Acetylamino-cyclohexanecarbonyl)-6-oxo-hexahydro-pyrrolo[3,2-b]pyrrole-1-carbonyl]-3-methyl-butyl}-4-diethylamino-benzamide;
2032. 4-Diethylamino-N-{1-[4-(1-hydroxy-cyclobutanecarbonyl)-6-oxo-hexahydro-pyrrolo[3,2-b]pyrrole-1-carbonyl]-3-methyl-butyl}-benzamide;
2033. 4-Diethylamino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
2034. 4-Diethylamino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
2035. 4-Diethylamino-N-{1-[4-(1-methoxy-cyclobutanecarbonyl)-6-oxo-hexahydro-pyrrolo[3,2-b]pyrrole-1-carbonyl]-3-methyl-butyl}-benzamide;
2036. 4-Diethylamino-N-{1-[4-(1-methoxy-cyclopentanecarbonyl)-6-oxo-hexahydro-pyrrolo[3,2-b]pyrrole-1-carbonyl]-3-methyl-butyl}-benzamide;
2037. 4-Diethylamino-N-{1-[4-(1-methoxy-cyclohexanecarbonyl)-6-oxo-hexahydro-pyrrolo[3,2-b]pyrrole-1-carbonyl]-3-methyl-butyl}-benzamide;
2038. N-{1-[4-(2-Amino-2-cyclopentyl-acetyl)-6-oxo-hexahydro-pyrrolo[3,2-b]pyrrole-1-carbonyl]-3-methyl-butyl}-4-diethylamino-benzamide;
2039. N-{1-[4-(2-Amino-2-cyclohexyl-acetyl)-6-oxo-hexahydro-pyrrolo[3,2-b]pyrrole-1-carbonyl]-3-methyl-butyl}-4-diethylamino-benzamide;
2040. N-{1-[4-(2-Acetylamino-2-cyclopentyl-acetyl)-6-oxo-hexahydro-pyrrolo[3,2-b]pyrrole-1-carbonyl]-3-methyl-butyl}-4-diethylamino-benzamide;
2041. N-{1-[4-(2-Acetylamino-2-cyclohexyl-acetyl)-6-oxo-hexahydro-pyrrolo[3,2-b]pyrrole-1-carbonyl]-3-methyl-butyl}-4-diethylamino-benzamide;
2042. N-{1-[4-(2-Acetylamino-propionyl)-6-oxo-hexahydro-pyrrolo[3,2-b]pyrrole-1-carbonyl]-3-methyl-butyl}-4-diethylamino-benzamide;
2043. N-{1-[4-(2-Acetylamino-acetyl)-6-oxo-hexahydro-pyrrolo[3,2-b]pyrrole-1-carbonyl]-3-methyl-butyl}-4-diethylamino-benzamide;
2044. N-(1-{4-[2-(Acetyl-methyl-amino)-acetyl]-6-oxo-hexahydro-pyrrolo[3,2-b]pyrrole-1-carbonyl}-3-methyl-butyl]-4-diethylamino-benzamide;
2045. 4-Diethylamino-N-{1-[4-(2-dimethylamino-acetyl)-6-oxo-hexahydro-pyrrolo[3,2-b]pyrrole-1-carbonyl]-3-methyl-butyl}-benzamide;
2046. N-{1-[4-(2-Amino-butyryl)-6-oxo-hexahydro-pyrrolo[3,2-b]pyrrole-1-carbonyl]-3-methyl-butyl}-4-diethylamino-benzamide;
2047. N-{1-[4-(2-Acetylamino-butyryl)-6-oxo-hexahydro-pyrrolo[3,2-b]pyrrole-1-carbonyl]-3-methyl-butyl}-4-diethylamino-benzamide;
2048. N-{1-[4-(2-amino-3-methyl-butyryl)-6-oxo-hexahydro-pyrrolo[3,2-b]pyrrole-1-carbonyl]-3-methyl-butyl}-4-diethylamino-benzamide;
2049. N-{1-[4-(2-Acetylamino-3-methyl-butyryl)-6-oxo-hexahydro-pyrrolo[3,2-b]pyrrole-1-carbonyl]-3-methyl-butyl}-4-diethylamino-benzamide;
2050. N-{1-[4-(2-amino-3,3-dimethyl-butyryl)-6-oxo-hexahydro-pyrrolo[3,2-b]pyrrole-1-carbonyl]-3-methyl-butyl}-4-diethylamino-benzamide;
2051. N-{1-[4-(2-Acetylamino-3,3-dimethyl-butyryl)-6-oxo-hexahydro-pyrrolo[3,2-b]pyrrole-1-carbonyl]-3-methyl-butyl}-4-diethylamino-benzamide;
2052. N-{1-[4-(2-Amino-3-methyl-pentanoyl)-6-oxo-hexahydro-pyrrolo[3,2-b]pyrrole-1-carbonyl]-3-methyl-butyl}-4-diethylamino-benzamide;
2053. N-{1-[4-(2-Acetylamino-3-methyl-pentanoyl)-6-oxo-hexahydro-pyrrolo[3,2-b]pyrrole-1-carbonyl]-3-methyl-butyl}-4-diethylamino-benzamide;
2054. 4-Diethylamino-N-[3-methyl-1-(6-oxo-4-pentanoyl-hexahydro-pyrrolo[3,2-b]pyrrole-1-carbonyl)-butyl]-benzamide;
2055. 4-Diethylamino-N-{3-methyl-1-[4-(3-methyl-pentanoyl)-6-oxo-hexahydro-pyrrolo[3,2-b]pyrrole-1-carbonyl]-butyl}-benzamide;
2056. 4-Diethylamino-N-{3-methyl-1-[4-(4-methyl-pentanoyl)-6-oxo-hexahydro-pyrrolo[3,2-b]pyrrole-1-carbonyl]-butyl}-benzamide;
2057. N-{1-[4-(2-Amino-4-methyl-pentanoyl)-6-oxo-hexahydro-pyrrolo[3,2-b]pyrrole-1-carbonyl]-3-methyl-butyl}-4-diethylamino-benzamide;
2058. N-{1-[4-(2-Acetylamino-4-methyl-pentanoyl)-6-oxo-hexahydro-pyrrolo[3,2-b]pyrrole-1-carbonyl]-3-methyl-butyl}-4-diethylamino-benzamide;
2059. N-{1-[4-(2-Amino-4,4-dimethyl-pentanoyl)-6-oxo-hexahydro-pyrrolo[3,2-b]pyrrole-1-carbonyl]-3-methyl-butyl}-4-diethylamino-benzamide;
2060. N-{1-[4-(2-Acetylamino-4,4-dimethyl-pentanoyl)-6-oxo-hexahydro-pyrrolo[3,2-b]pyrrole-1-carbonyl]-3-methyl-butyl}-4-diethylamino-benzamide;
2061. N-{1-[4-(2-Amino-pentanoyl)-6-oxo-hexahydro-pyrrolo[3,2-b]pyrrole-1-carbonyl]-3-methyl-butyl}-4-diethylamino-benzamide;
2062. N-{1-[4-(2-Acetylamino-pentanoyl)-6-oxo-hexahydro-pyrrolo[3,2-b]pyrrole-1-carbonyl]-3-methyl-butyl}-4-diethylamino-benzamide;
2063. N-{1-[4-(2-Amino-5-methyl-hexanoyl)-6-oxo-hexahydro-pyrrolo[3,2-b]pyrrole-1-carbonyl]-3-methyl-butyl}-4-diethylamino-benzamide;
2064. N-{1-[4-(2-Acetylamino-5-methyl-hexanoyl)-6-oxo-hexahydro-pyrrolo[3,2-b]pyrrole-1-carbonyl]-3-methyl-butyl}-4-diethylamino-benzamide;
2065. N-{1-[4-(2-hydroxy-3-methyl-butyryl)-6-oxo-hexahydro-pyrrolo[3,2-b]pyrrole-1-carbonyl]-3-methyl-butyl}-4-diethylamino-benzamide;
2066. N-{1-[4-(2-methoxy-3-methyl-butyryl)-6-oxo-hexahydro-pyrrolo[3,2-b]pyrrole-1-carbonyl]-3-methyl-butyl}-4-diethylamino-benzamide;
2067. 4-Diethylamino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
2068. 4-Diethylamino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
2069. 4-Diethylamino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
2070. 4-Diethylamino-N-{1-[4-(2-methoxy-3-methyl-pentanoyl)-6-oxo-hexahydro-pyrrolo[3,2-b]pyrrole-1-carbonyl]-3-methyl-butyl}-benzamide,
2071. 4-Diethylamino-N-{1-[4-(2-hydroxy-4-methyl-pentanoyl)-6-oxo-hexahydro-pyrrolo[3,2-b]pyrrole-1-carbonyl]-3-methyl-butyl}-benzamide;
2072. 4-Diethylamino-N-{1-[4-(2-methoxy-4-methyl-pentanoyl)-6-oxo-hexahydro-pyrrolo[3,2-b]pyrrole-1-carbonyl]-3-methyl-butyl}-benzamide;
2073. 4-Diethylamino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
2074. 4-Diethylamino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
2075. 4-Diethylamino-N-{1-[4-(2-hydroxy-pentanoyl)-6-oxo-hexahydro-pyrrolo[3,2-b]pyrrole-1-carbonyl]-3-methyl-butyl}-benzamide;
2076. 4-Diethylamino-N-{1-[4-(2-methoxy-pentanoyl)-6-oxo-hexahydro-pyrrolo[3,2-b]pyrrole-1-carbonyl]-3-methyl-butyl}-benzamide;
2077. 4-Diethylamino-N-{1-[4-(2-hydroxy-5-methyl-hexanoyl)-6-oxo-hexahydro-pyrrolo[3,2-b]pyrrole-1-carbonyl]-3-methyl-butyl}-benzamide;
2078. 4-Diethylamino-N-{1-[4-(2-methoxy-5-methyl-hexanoyl)-6-oxo-hexahydro-pyrrolo[3,2-b]pyrrole-1-carbonyl]-3-methyl-butyl}-benzamide;

To those skilled in the practices of organic chemistry, compounds of general formula (I) may be readily synthesised by a number of chemical strategies, performed either in solution or on the solid phase (see Atherton, E. and Sheppard, R. C. In ‘Solid Phase Peptide Synthesis: A Practical Approach’, Oxford University Press, Oxford, U.K. 1989, for a general review of solid phase synthesis principles). The solid phase strategy is attractive in being able to generate many thousands of analogues, typically on a 5-100 mg scale, through established parallel synthesis methodologies (e.g. see (a) Bastos, M.; Maeji, N. J.; Abeles, R. H. Proc. Natl. Acad. Sci. USA, 92, 6738-6742, 1995).

Therefore, one strategy for the synthesis of compounds of general formula (I) comprises:—
(a) Preparation of an appropriately functionalised and protected bicyclic ketone building block in solution.
(b) Attachment of the building block (a) to the solid phase through a linker that is stable to the conditions of synthesis, but readily labile to cleavage at the end of a synthesis (see James, I. W., Tetrahedron, 55(Report No 489), 4855-4946, 1999, for examples of the ‘linker’ function as applied to solid phase synthesis).
(c) Solid phase organic chemistry (see Brown, R. D. J. Chem. Soc., Perkin Trans.1, 19, 3293-3320, 1998), to construct the remainder of the molecule.
(d) Compound cleavage from the solid phase into solution.
(e) Cleavage work-up and compound analysis.

The first stage in a synthesis of compounds of general formula (I) is the preparation in solution of a functionalised and protected building block. Typical schemes towards the hexahydropyrrolo[3,2-b]pyrrol-3-one (6) are detailed in Schemes 1-3, a hexahydropyrrolo[3,2-c]pyrazol-6-one (21) in Scheme 4 and a hexahydro-2-oxa-1,4-diazapentalen-6-one (26) in Scheme 5. The synthetic descriptions detailed in Schemes 6-18 could equally be applied using each of the scaffolds of general formula (I).

‘Pg₁’ and ‘Pg₂’ denotes suitable amine protecting groups such as the 9-fluorenyl methoxycarbonyl (Fmoc, see Atherton, E. and Sheppard, R. C. In ‘Solid Phase Peptide Synthesis: A Practical Approach’, Oxford University Press, Oxford, U.K. 1989), tert-butoxycarbonyl (Boc), benzyloxycarbonyl (Cbz) or allyloxycarbonyl (Alloc) for example.

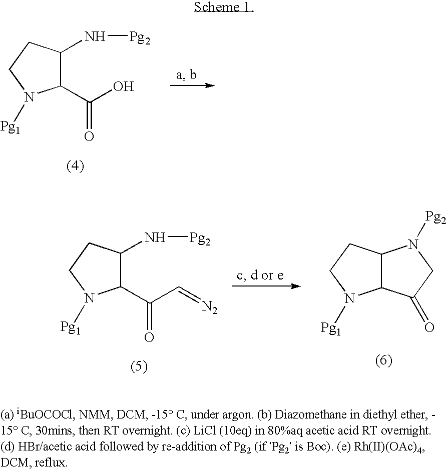

In the illustrated case, condensation with diazomethane provides Z=CH₂ in general formula (I). Synthesis may commence from a suitably protected β-aminoproline (4) which are described in the literature e.g. Gomez-Vidal, J. A. and Silverman, R. B. Org. Lett., 3(16), 2481-2484, 2001.

Activation of the suitably protected β-aminoproline (4) via isobutyl chloroformate mixed anhydride, followed by condensation with diazomethane, yields the diazomethylketone intermediate (5). Treatment of diazomethylketone intermediate (5) with lithium chloride in aqueous acetic acid provides the protected hexahydropyrrolo[3,2-b]pyrrol-3-one (6). Alternatively, when Pg₂ is Boc, treatment with HBr in acetic acid provides an intermediate bicycle with the secondary amine. HBr salt. This intermediate may be acylated with a variety of reagents e.g. activated carboxylic acids, sulphonyl chlorides, urethane chloroformates to provide many variations of (6) where the nitrogen substituent is a suitable protecting group ‘Pg₂’ or R²C(O), R²SO₂, etc. Alternatively, treatment of diazomethylketone intermediate (5) with rhodium (II) tetraacetate in dichloromethane provides the hexahydropyrrolo[3,2-b]pyrrol-3-one (6) (e.g. see Lall, M. S. et al, J. Org. Chem., 67, 1536-1547, 2002. and refs cited therein).

Introduction of simple ‘Z’ substituents may be achieved by condensation of activated (4) with alternatives to diazomethane such as diazoethane (Z=CHCH₃, R³=H, R⁴=CH₃), or I-phenyloxydiazoethane (Z=CHCH₂OPh, R³=H, R⁴=CH₂OPh).

An alternative route towards a suitably protected building block is detailed in Scheme 2. Using an Arndt-Eistert synthesis, a suitably protected 3,4-dehydroproline (7) may be homologated by methylene insertion between the α-carbon and carboxylic acid following standard literature methods (e.g. see Meier and Zeller, Angew. Chem. Intl. Ed. Engl., 14, 3243, 1975 for a review). Conversion of (7) into the α-diazomethylketone proceeds via isobutyl chloroformate mixed anhydride, followed by condensation with diazomethane. Wolff rearrangement, e.g. with silver oxide in methanol provides the protected homologated analogue (8), e.g. 2-Methoxycarbonylmethyl-2,5-dihydro-pyrrole-1-carboxylic acid tert-butyl ester.

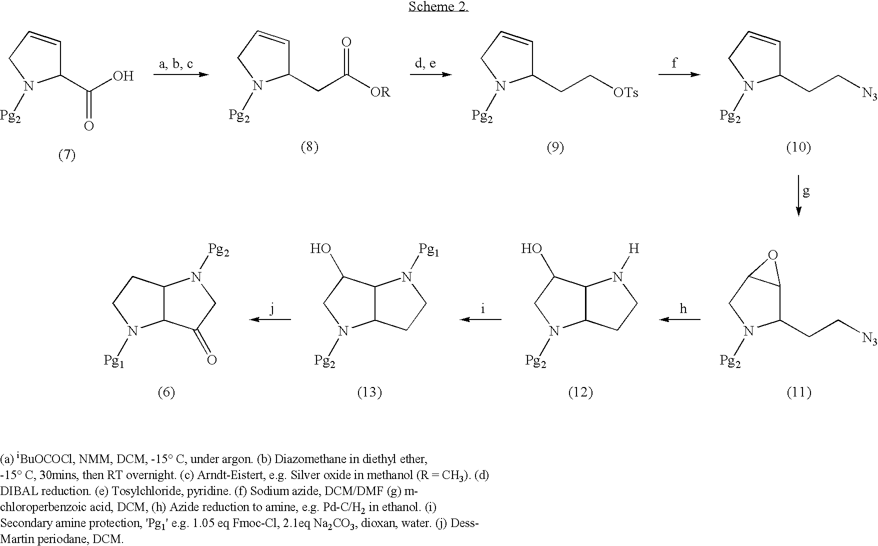

Treatment of the methyl ester (8) with a reducing agent such as DIBAL-H (diisobutylaluminium hydride) provides the primary alcohol, which is readily converted to tosylate (9). Similarly the mesylate or triflate analogues of (9) may be prepared. Nucleophilic displacement of the activated alcohol with sodium azide provides intermediate (10) e.g. 2-(2-Azido-ethyl)-2,5-dihydro-pyrrole-1-carboxylic acid tert-butyl ester. Epoxidation of (10) with oxidising agents common to the art such as m-CPBA provides the epoxide (11) e.g. 2-(2-Azido-ethyl)-6-oxa-3-aza-bicyclo[3.1.0]hexane-3-carboxylic acid tert-butyl ester. Reduction of the azide (11) to the amine intermediate may be effected under a range of conditions such as Pd—C/H₂ or triphenylphosphine in THF and water. The amine intermediates undergo intramolecular epoxide ring opening to provide the bicyclic alcohol (12) e.g. 3-Hydroxy-hexahydro-pyrrolo[3,2-b]pyrrole-1-carboxylic acid tert-butyl ester. The free secondary amine (12) may be protected with a variety of suitable protecting groups such as Fmoc, Boc, Cbz, Alloc to provide orthogonal protection of the bicyclic scaffold. Protected alcohol (13) may be oxidised by reagents common to the art such as pyridine sulphur trioxide complex in DMSO and triethylamine or Dess-Martin periodane to provide ketone (6) e.g. 3-Oxo-hexahydro-pyrrolo[3,2-b]pyrrole-1,4-dicarboxylic acid 1-tert-butyl ester 4-(9H-fluoren-9-ylmethyl) ester.

Alternative routes towards intermediate (9) (ex Scheme 2) are available such as that detailed in Scheme 3. Protected alkene (14) is readily available following literature procedures from the protected homoserine lactol ((a) Wright, D. L. et al, Org. Lett., 2(13), 1847-1850, 2000. (b) Boyle, P. H. et al, Tet. Asymm., 6, 2819, 1995. (c) Baldwin, J. E. and Flinn, A., Org. Lett., 2, 3605, 1987.). N-alkylation of (14) with a base such as sodium hydride and allyl bromide provides diene (15). Treatment of (15) with the olefin metathesis catalysts developed by Grubbs such as bis(tricyclohexylphosphine)benzylidine ruthenium (I) dichloride provides the protected primary alcohol intermediate of compound (9) detailed in Scheme 2.

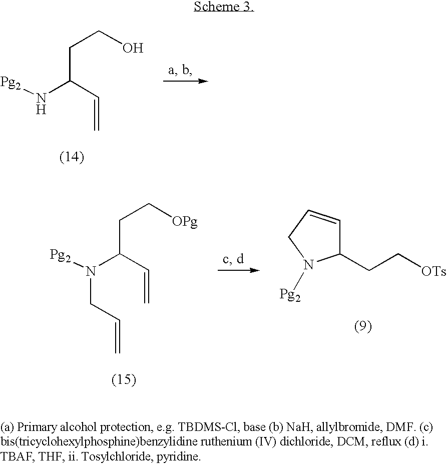

The hexahydropyrrolo[3,2-c]pyrazol-6-one (21) scaffold may be prepared following a similar route to that described in Scheme 2 (see Scheme 4). Treatment of the protected 3,4-dehydroproline (7) with HCl in methanol provides the methyl ester. Reduction of the ester with a reducing agent such as DIBAL-H (diisobutylaluminium hydride) provides the primary alcohol, which is readily converted to tosylate (16). Similarly the mesylate or triflate analogues of (16) may be prepared. Nucleophilic displacement of the activated alcohol with a protected hydrazide e.g. Hydrazinecarboxylic acid allyl ester (Alloc-NHNH₂) followed by Boc protection of the secondary hydrazide e.g. under standard Schotten-Baumenn conditions and removal of the alloc group e.g. (PPh₃)-4Pd^{o}/DCM/PhSiH₃ provides (17). Epoxidation of (17) with oxidising agents common to the art such as m-CPBA provides the epoxide intermediate (18). Intermediate (18) readily undergoes intramolecular epoxide ring opening to provide the bicyclic alcohol (19). The free secondary hydrazide (19) may be protected with a variety of suitable protecting groups e.g. Fmoc, Cbz, Alloc to provide orthogonal protection of the bicyclic scaffold. Protected alcohol (20) may be oxidised by reagents common to the art such as pyridine sulphur trioxide complex in DMSO and triethylamine or Dess-Martin periodane to provide ketone (21).

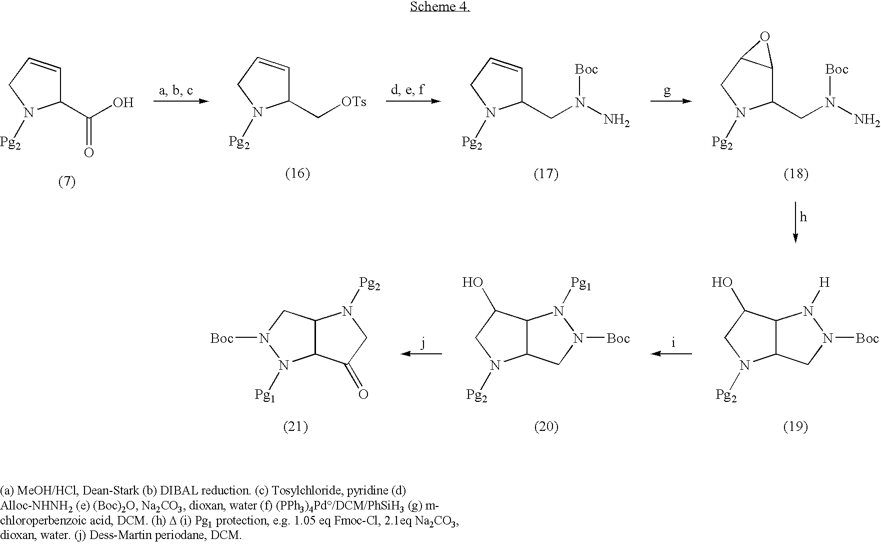

The hexahydro-2-oxa-1,4-diazapentalen-6-one (26) scaffold may be prepared following a similar route to that described in Scheme 4. Tosylate (16) undergoes nucleophilic displacement with a protected oxyamine e.g. N-Boc hydroxylamine to provide intermediate (22). Epoxidation of (22) with oxidising agents common to the art such as m-CPBA provides the epoxide intermediate, which upon acidolytic removal of the Boc group provides intermediate amine.salt (23). Neutralisation of the amine.salt initiates intramolecular epoxide ring opening to provide the bicyclic alcohol (24). The free secondary amine (24) may be protected with a variety of suitable protecting groups e.g. Fmoc, Cbz, Alloc, Boc to provide orthogonal protection of the bicyclic scaffold. Protected alcohol (25) may be oxidised by reagents common to the art such as pyridine sulphur trioxide complex in DMSO and triethylamine or Dess-Martin periodane to provide ketone (26) e.g. 6-Oxo-tetrahydro-2-oxa-1,4-diaza-pentalene-1,4-dicarboxylic acid 4-benzyl ester 1-(9H-fluoren-9-ylmethyl) ester.

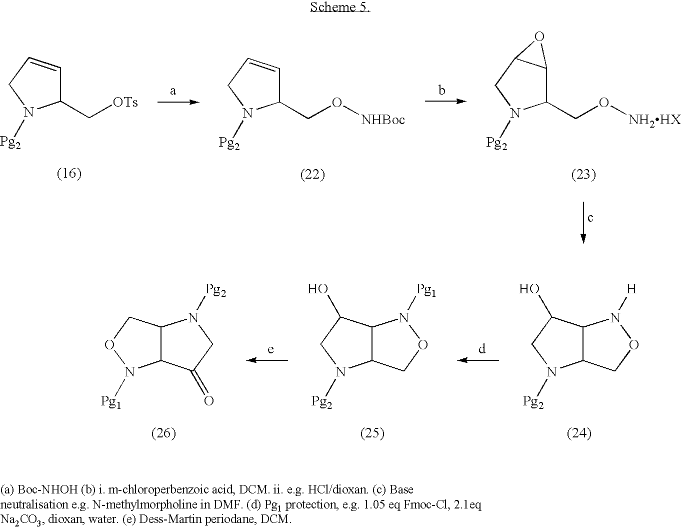

The protected building blocks detailed in Schemes 1, 2, 4 and 5 may be utilised in a solid phase synthesis of inhibitor molecules (steps (b) to (e)). Preferred protecting group combinations include ‘Pg₁’=Fmoc/‘Pg₂’=Boc, or ‘Pg₁’=Fmoc/‘Pg₂’=Alloc, or ‘Pg₁’=Boc/‘Pg₂’=Fmoc, or ‘Pg₁’=Boc/‘Pg₂’=Alloc, or ‘Pg₁’=Alloc/‘Pg₂’=Fmoc, or ‘Pg₁’=Alloc/‘Pg₂’=Boc. The solid phase syntheses exemplified in Schemes 6 and 7 by the use of hexahydropyrrolo[3,2-b]pyrrol-3-one (6) and octahydropyrrolo[3,2-b]pyrrol-3-ol (13) could equally apply to ketones (21) and (26) and alcohols (20) and (25). Step (b), the solid phase linkage of an aldehyde or ketone, has previously been described by a variety of methods (e.g. see (a) James, I. W., 1999, (b) Lee, A., Huang, L., Ellman, J. A., J. Am. Chem. Soc, 121(43), 9907-9914, 1999, (c) Murphy, A. M., et al, J. Am. Chem. Soc, 114, 3156-3157, 1992). A suitable method amenable to the reversible linkage of an alkyl ketone functionality such as (6) is through a combination of the previously described chemistries. The semicarbazide, 4[[hydrazinocarbonyl)amino]methyl]cyclohexane carboxylic acid. trifluoroacetate (Murphy, A. M., et al, J. Am. Chem. Soc, 114, 3156-3157, 1992), may be utilised as illustrated in Scheme 6, where ‘Pg₁’=Fmoc and ‘Pg₂’=Boc or Alloc, exemplified by linkage of the hexahydropyrrolo[3,2-b]pyrrol-3-one (6).

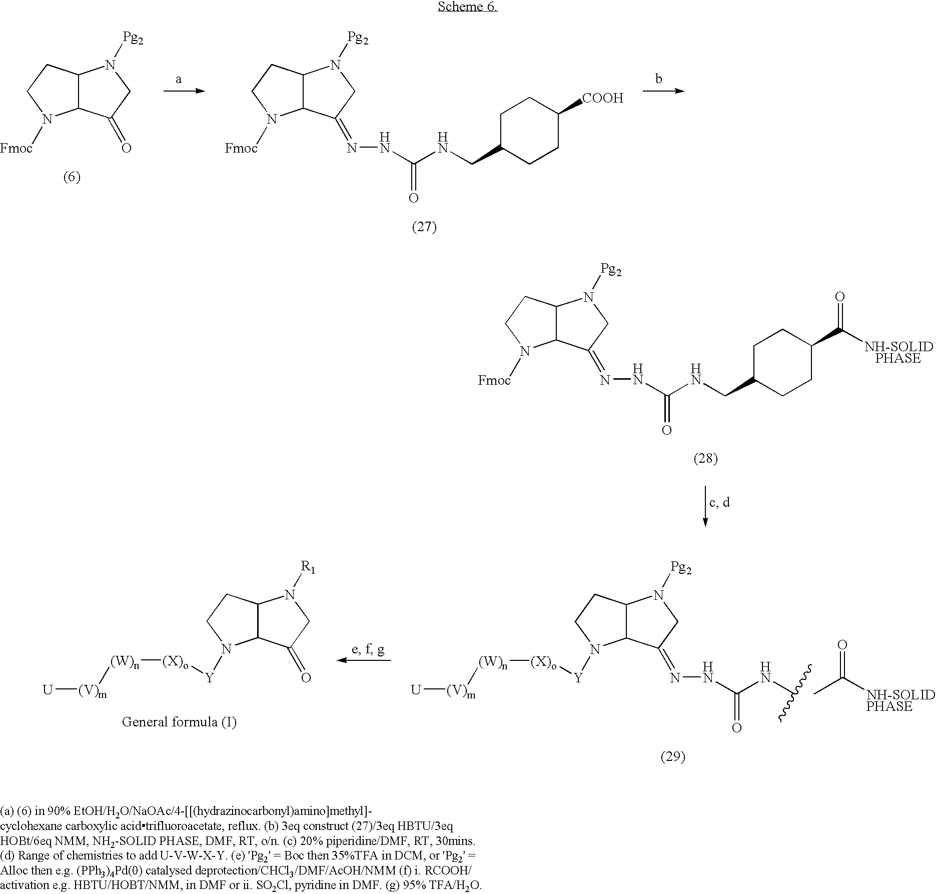

Construct (28) is prepared through reaction of the linker molecule (27) and the hexahydropyrrolo[3,2-b]pyrrol-3-one (6) by reflux in aqueous ethanol/sodium acetate. Standard solid phase techniques (e.g. see Atherton, E. and Sheppard, R. C. In ‘Solid Phase Peptide Synthesis: A Practical Approach’, Oxford University Press, Oxford, U.K. 1989) are used to anchor the construct to an amino-functionalised solid phase through the free carboxylic acid functionality of (27), providing the loaded construct (28). Loaded construct (28) may be reacted with a wide range of carboxylic acids or sulphonyl chlorides available commercially in the literature, to introduce the left-hand portion ‘U-V-W-X-Y’ in general formula (I), providing loaded construct (29). Orthogonal removal of ‘Pg₂’ then liberates the secondary amine functionality of the right-hand ring, which may be acylated with a range of carboxylic acid and sulphonyl chlorides. Finally, compounds of general formula (I) are released from the solid phase by treatment with 95% aq trifluoroacetic acid.

An alternative solid phase synthesis of compounds of general formula (I) utilises the bicyclic alcohol intermediate (13), Scheme 7. The secondary alcohol may be attached to the solid phase through the acid labile dihydropyran linker (30) that is well known in the literature (e.g. see (a) Thompson, L. A. and Ellman, J. A., Tet. Lett., 35, 9333, 1994. (b) Kick, E. K and Ellman, J. A. J. Med. Chem., 38, 1427, 1995.). Preferred protecting group combinations include ‘Pg₁’=Fmoc/‘Pg₂’ Alloc, or ‘Pg₁’=Alloc/‘Pg₂’=Fmoc.

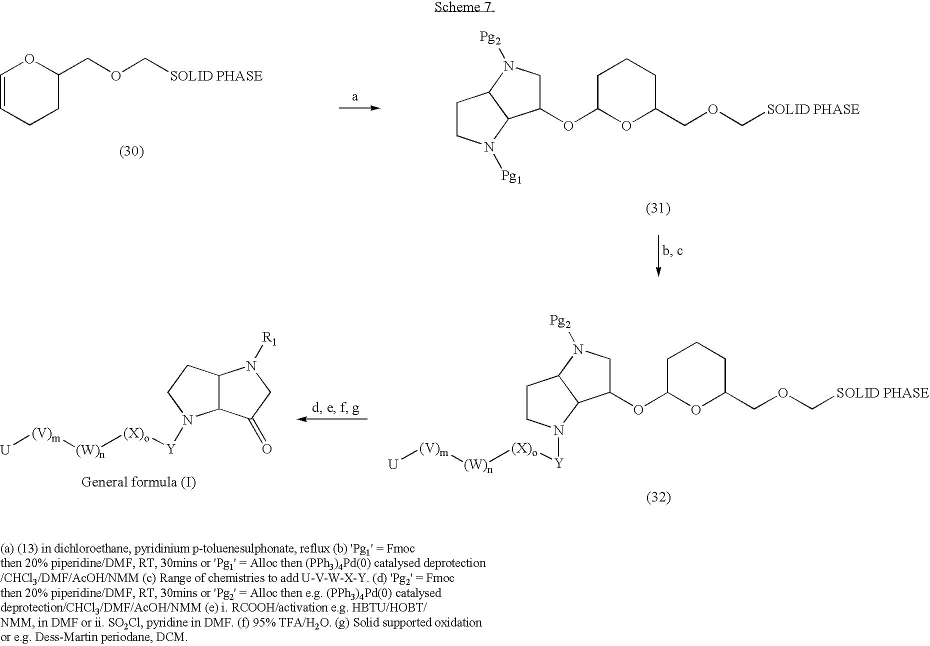

Loaded construct (31) may be reacted with a wide range of carboxylic acids or sulphonyl chlorides available commercially in the literature, to introduce the left-hand portion ‘U-V-W-X-Y’ in general formula (I), providing loaded construct (32). Orthogonal removal of ‘Pg₂’ then liberates the secondary amine functionality of the right-hand ring, which may be acylated with a range of carboxylic acid and sulphonyl chlorides. Compounds of general formula (I) are released from the solid phase by treatment with 95% aq trifluoroacetic acid and the resultant alcohols may be oxidised with a range of solution based reagents e.g. Dess-Marin periodane in DCM or solid supported oxidants (e.g. see Ley, S. V. et al, J. Chem. Soc. Perkin Trans. 1., 3815-4195, 2000.) to provide the ketone products.

In the simplest example, the entire left hand portion of an inhibitor of general formula (I) comprises a capped amino acid (Scheme 8), providing for example analogues of general formula (I) where Z=‘CH₂’, Y=CHR¹¹C(O), (X)ₒ=‘-’, (W)ₙ=‘NH’, R¹⁸=‘H’, n=1, (V)ₘ=‘CO’, m=1 and U=phenyl. Scheme 8 details chemistry utilising protected ketone construct (33) and the reactions could equally be applied to the protected alcohol construct (31).

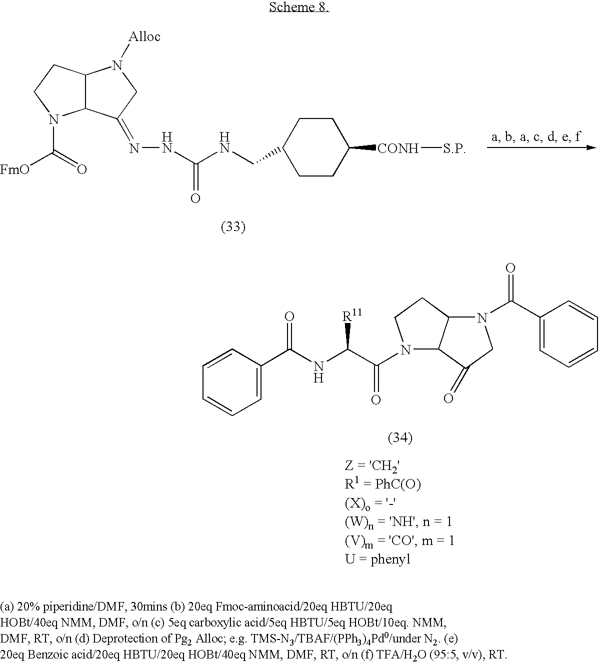

Alternatively, carboxylic acids can be prepared in solution by traditional organic chemistry methods and coupled to constructs (28) and (31) on the solid phase (Schemes 9-13). For example (Scheme 9), treatment in solution of an amino acid, exemplified by (35) with sodium nitrite/H₂SO₄, provides the α-hydroxyacid, exemplified by (36) (Degerbeck, F. et al, J. Chem. Soc, Perkin Trans. 1, 11-14, 1993). Treatment of α-hydroxyacid, (36) with sodium hydride in a dimethylformamide/dichloromethane mixture followed by addition of benzyl bromide, provides (RS) 2-benzyloxymethyl-pentanoic acid (37). Coupling of (37) to the solid phase construct (33) followed by alloc deprotection, benzoylation then cleavage, provides (38), an example of general formula (I) where Z=‘CH₂’, (X)ₒ=‘-’, (W)ₙ=‘O’, n=1, (V)ₘ=‘CH₂’, m=1, R¹⁹ and R²⁰=H and U=phenyl. To those skilled in the practices of organic synthesis, a wide variety of aminoacids such as (35) may be converted to the corresponding α-hydroxyacid such as (36) following the general conditions detailed. Additionally, benzylbromide may be replaced by any reasonable Ar—CR¹⁹R²⁰-halide, providing many variations of carboxylic acid (37) following the general conditions detailed. In certain instances, it may be advantageous to temporarily protect the carboxylic acid as the methyl ester (for example compound (43), Scheme 11) prior to reaction with the alkylhalide. The ester intermediate is then simply hydrolysed to acid (37). Analogues of (38), exploring a wide range of (V)ₘ and U in general formula (I) may be prepared through the general conditions detailed in Scheme 9.

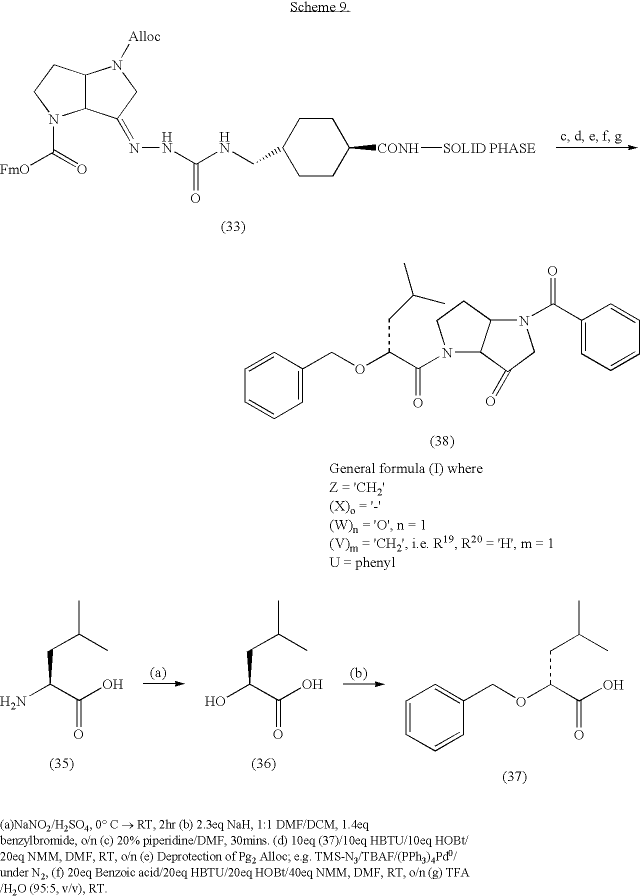

Since the final synthetic step involves a trifluoroacetic acid (TFA) mediated cleavage of the solid phase bound compound, analogues where the substituted ether is labile to TFA may be prepared in solution by an alternative route (see Scheme 16).

Alternatively, coupling of construct (33) (following removal of Fmoc) with the α-hydroxyacid (36), provides a versatile solid phase bound intermediate ‘Y’ substituent in general formula (I) that may be reacted with many reagents. For example, the α-hydroxyl can be reacted under Mitsunobu conditions (Hughes, D. L. Org. React. (N.Y), 42, 335-656, 1992) to give ethers (i.e. X=‘-’, W=‘O’, in general formula (I)) (see Grabowska, U. et al, J. Comb. Chem., 2(5), 475-490, 2000, for an example of Mitsunobu reaction on the solid phase). Alternatively, the α-hydroxyl can be reacted with a carbamoyl chloride to give a carbamate (i.e. X=‘-’, W=‘O’, V=‘NHC(O)’, in general formula (I)).

Alternatively, (Scheme 10), treatment in solution of an amino acid, exemplified by (35) with sodium nitrite/H₂SO₄/potassium bromide provides the α-bromoacid, exemplified by (39) (Souers, A. J. et al, Synthesis, 4, 583-585, 1999) with retention of configuration. Treatment of α-bromoacid (39) with an alkylthiol exemplified by 4-tert-butylphenylmethanethiol (40) in dimethylformamide/triethylamine, provides 2R-(4-tert-butylbenzylsulfanyl)-4-methylpropionic acid (41), with inversion of configuration. Coupling of (41) to the solid phase construct (33) followed by alloc deprotection, benzoylation, then cleavage provides (42), an example of general formula (I) where Z=‘CH₂’, (X)ₒ=‘-’, (W)ₙ=‘S’, n=1, (V)ₘ=‘CH₂’, m=1, R¹⁹ and R²⁰=H and U=4-tert-butylphenyl. To those skilled in the practices of organic synthesis, a wide variety of aminoacids such as (35) may be converted to the corresponding α-bromoacid such as (39) following the general conditions detailed. Additionally, starting with the R-isomer of (35) gives the R-bromoacid analogue of (39) and S-thioether analogue of (41). Additionally, (4-tert-butylphehyl)methanethiol (40) may be replaced by any reasonable Ar—CR¹⁹R²⁰—SH, providing many variations of carboxylic acid (41) following the general conditions detailed. Thus analogues of (42) exploring a wide range of (V)ₘ and U in general formula (I) may be prepared through the general conditions detailed in Scheme 10.

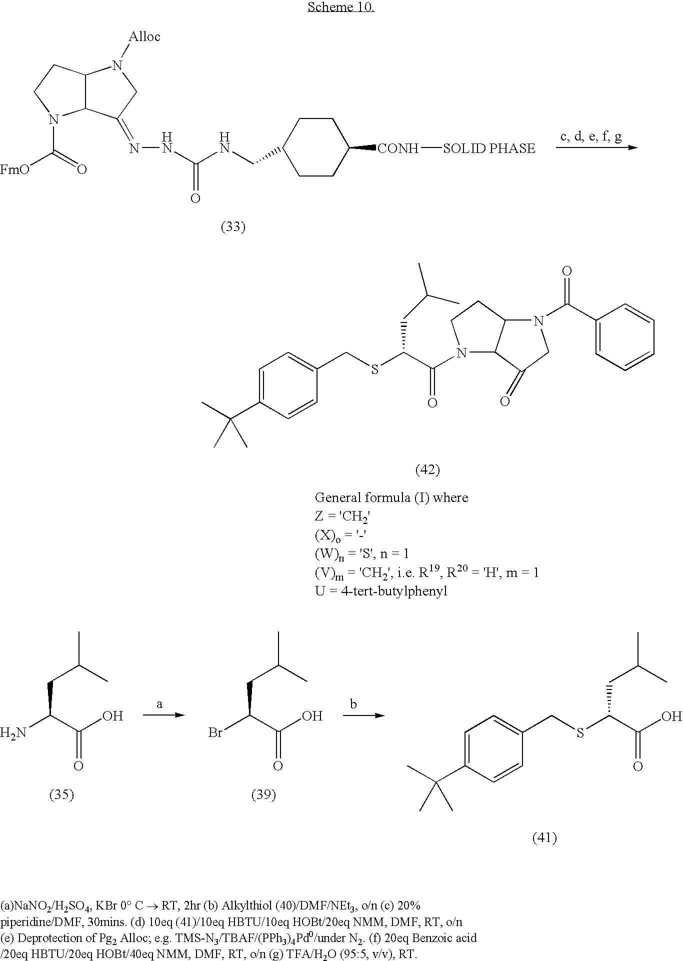

Alternatively, coupling of construct (33) (following removal of Fmoc) with an α-bromoacid e.g. (39), provides a versatile intermediate ‘Y’ substituent in general formula (I) that may be reacted with many reagents. For example, the α-bromide can be displaced with nucleophiles e.g. alcohols, thiols, carbanions etc, to give ethers (i.e. X=‘-’, W=‘O’, in general formula (I)), thioethers (i.e. X=‘-’, W=‘S’, in general formula (I)). The thioethers may optionally be oxidised to the sulphone (see Scheme 14, i.e. X=‘-’, W=‘SO₂’, in general formula (I)) (see Grabowska, U. et al, J. Comb. Chem., 2(5), 475-490, 2000, for an example of bromide displacement and thioether oxidation on the solid phase).

Alternatively, (Scheme 11), treatment of an α-hydroxyacid, exemplified by (36) with trimethylsilylchloride and methanol provides the methyl ester (43). Activation of the free hydroxyl to the chloroformate with phosgene in dichloromethane followed by addition of morpholine, then hydrolysis, provides morpholin-4-carboxylic acid 1-carboxy-3-methyl-butyl ester (44). Coupling of (44) to the solid phase construct (33) followed by alloc deprotection, benzoylation then cleavage provides (45), an example of general formula (I) where Z=‘CH₂’, (X)ₒ=‘-’, (W)_{n=‘O’, n=}1, (V)ₘ=‘CO’ and U=morpholino. To those skilled in the practices of organic synthesis, a wide variety of α-hydroxyacid esters such as (36) could be converted to the activated chloroformate following the general conditions detailed. Additionally, morpholine may be replaced by any reasonable amine, providing many variations of carboxylic acid (44) following the general conditions detailed. Thus analogues of (45) exploring a wide range of (V)ₘ and U in general formula (I) may be prepared through the general conditions detailed in Scheme 11.

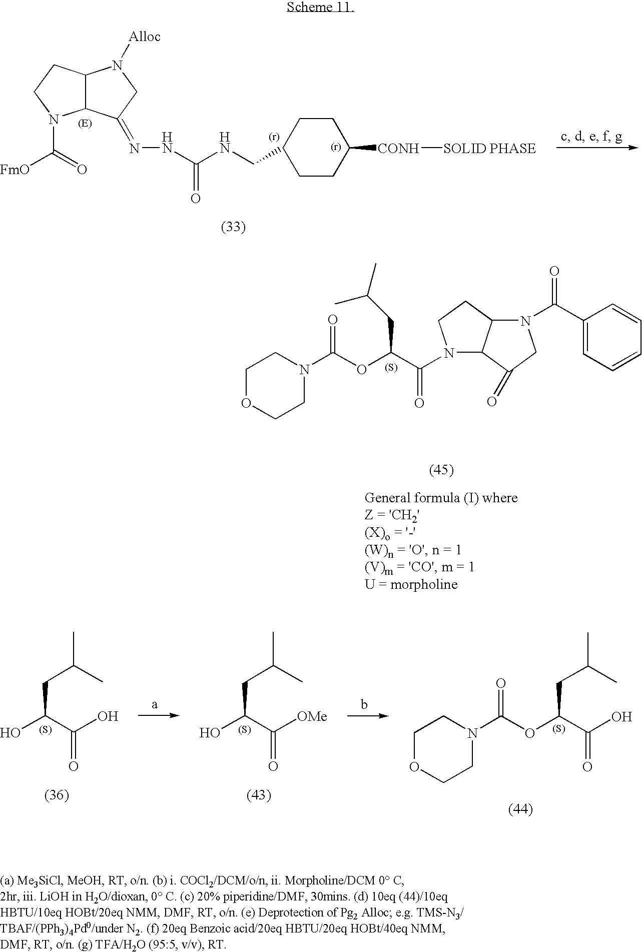

Alternatively, (Scheme 12), a wide range of alkylsuccinate esters exemplified by 2R-isobutyl-succinic acid 1-methyl ester (46) are commercially available or readily prepared by known methods (see (a) Azam et al, J. Chem. Soc. Perkin Trans. 1, 621-, 1996; (b) Evans et al, J. Chem. Soc. Perkin Trans. 1, 103, 2127, 1981; (c) Oikawa et al, Tet. Lett, 37, 6169, 1996). Carboxyl activation of alkylsuccinate ester (46) followed by addition of morpholine in dimethylformamide and subsequent ester hydroylsis, provides 4-Methyl-2R-(2-morpholin-4-yl-2-oxo-ethyl)-pentanoic acid (47). Coupling of (47) to the solid phase construct (33) followed by alloc deprotection, benzoylation then cleavage provides (48), an example of general formula (I) where Z=‘CH₂’, (X)ₒ=‘CH₂’, (W)ₙ=‘CO’, n=1, (V)ₘ=‘-’ and U=morpholino. To those skilled in the practices of organic synthesis, a wide variety of alkylsuccinate esters such as (46) may be prepared and converted to the corresponding substituted alkylsuccinate acid such as (47) following the general conditions detailed. Additionally, morpholine may be replaced by any reasonable amine, providing many variations of carboxylic acid (47) following the general conditions detailed. Thus analogues of (48) exploring a wide range of (X)ₒ, (V)ₘ and U in general formula (I) may be prepared through the general conditions detailed in Scheme 12.

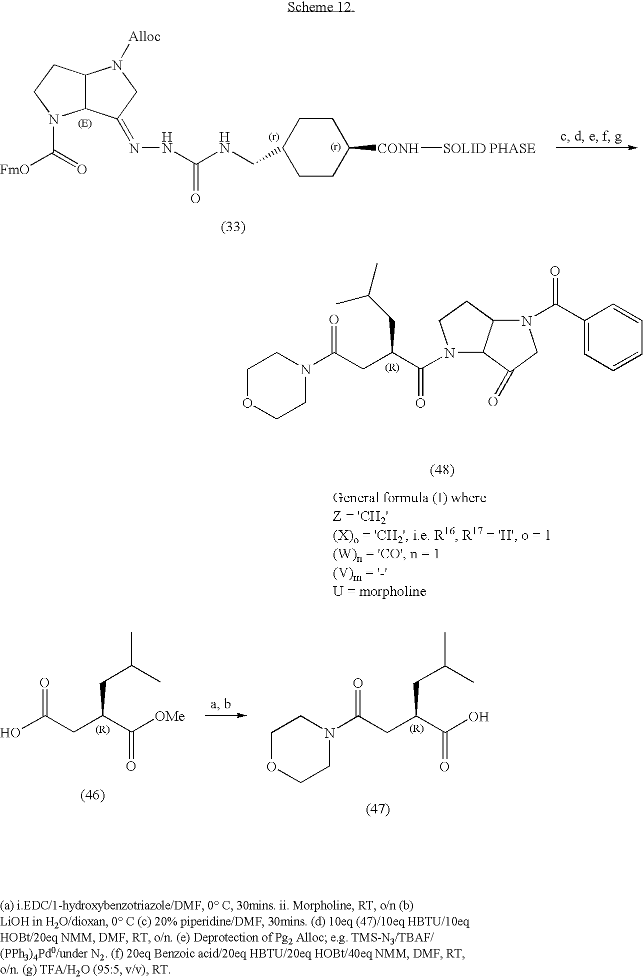

Alternatively, (Scheme 13), a wide range of biarylalkylacetic acids, exemplified by 2RS-biphenyl-3-yl-4-methylpentanoic acid (50) are readily available by known methods (see (a) DesJarlais, R. L. et al, J. Am. Chem. Soc, 120, 9114-9115, 1998; (b) Oballa, R. M. et al, WO 0149288). Coupling of biarylalkylacetic acid (50) to the solid phase construct (33) followed by alloc deprotection, benzoylation then cleavage provides (51), an example of general formula (I) where Z=‘CH₂’, (X)ₒ=‘-’, (W)ₙ=‘-’, (V)ₘ=‘-’ and U=m-biphenyl. To those skilled in the practices of organic synthesis, a wide variety of biarylalkylacetic acids such as (50) may be prepared by alkylation of the α-anion of the free acid analogue of (49), which in turn is prepared by Suzuki coupling of phenylboronic acid and 3-bromophenylacetic acid methyl ester. Phenylboronic acid may be replaced by a wide range of arylboronic acids in the Suzuki coupling, providing many variations of carboxylic acid (50) following the general conditions detailed. Thus analogues of (51) exploring a wide range of group ‘U’ in general formula (I) may be prepared through the general conditions detailed in Scheme 13.

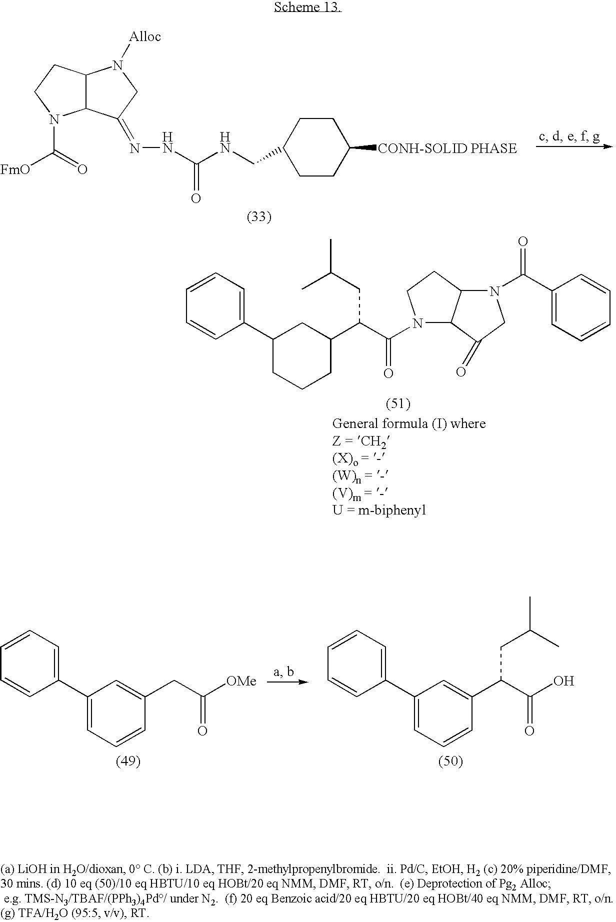

Many other possibilities for solid phase organic chemistry (e.g. see Brown, R. D. J. Chem. Soc., Perkin Trans. 1, 19, 3293-3320, 1998, for a review of recent SPOC publications) can be used to derivatise constructs (28) and (31) towards compounds of general formula (I). For example, the left-hand portion ‘U-V-W-X-Y’ in general formula (I) can be partially constructed in solution, coupled to constructs (28) and (31) and further modified on the solid phase. For example (Scheme 14), a simple extension of Scheme 10 is through the oxidation of the intermediate solid phase bound species, with m-chloroperbenzoic acid in dichloromethane prior to cleavage, to give the sulphone analogue. Commencing from 2R-(4-tert-butylbenzylsulfanyl)-4-methylpropionic acid (41), sulphone (52) is prepared, an example of general formula (I) where Z=‘CH₂’, (X)ₒ=‘-’, (W)ₙ=‘SO₂’, n=1, (V)ₘ=‘CH₂’, m=1, R¹⁹ and R²⁰=H and U=4-tert-butylphenyl. As described in Scheme 10, many variations of carboxylic acid (41) may be prepared following the general conditions detailed. Thus analogues of (52) exploring a wide range of (V)ₘ and U in general formula (I) may be prepared through the general conditions detailed in Schemes 10 and 14.

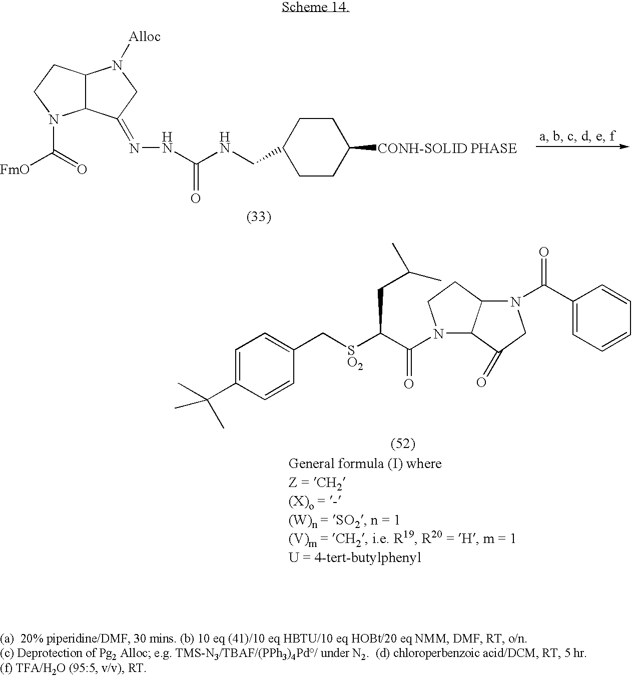

Compounds of general formula (I) are finally released from the solid phase by treatment with trifluoroacetic acid/water, followed by evaporation, lyophylis and standard analytical characterisation.

A second strategy for the synthesis of compounds of general formula (I) comprises:—
(a) Preparation of an appropriately functionalised and protected hexahydropyrrolo[3,2-b]pyrrol-3-one, hexahydropyrrolo[3,2-c]pyrazol-6-one or hexahydro-2-oxa-1,4-diazapentalen-6-one building block in solution.
Preferred protecting groups for solution phase chemistry are the 9-fluorenylmethoxycarbonyl, Nα-tert-butoxycarbonyl, Nα-benzyloxy carbonyl and Nα-allyloxycarbonyl group.
(b) Standard organic chemistry methods for the conversion of building block (a) towards compounds of general formula (I).

In the simplest example, the entire left hand portion of an inhibitor of general formula (I) can be prepared in solution by traditional organic chemistry methods and coupled to building block (a) (see Scheme 15 exemplified by the use of 3-Oxo-hexahydro-pyrrolo[3,2-b]pyrrole-1-carboxylic acid allyl ester (54)).

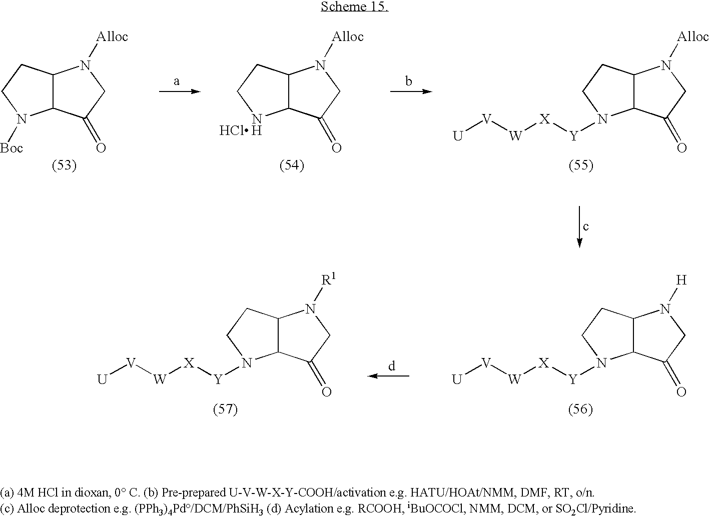

The general strategy detailed in Scheme 15 is particularly useful when the compound of general formula (I) contains a substituent that is labile to trifluoroacetic acid, this being the final reagent used in each of the solid phase Schemes 6-14. For example (Scheme 16), treatment in solution of α-hydroxyacid (36) with sodium hydride in a dimethylformamide/dichloromethane mixture followed by addition of 4-tert-butylbenzyl bromide, provides 2RS-(4-tert-butylbenzyloxy)-4-methylpentanoic acid (58). Coupling of (58) to hydrochloride salt (54), followed by alloc deprotection then benzoylation provides (59), an example of general formula (I) where Z=‘CH₂’, (X)ₒ=‘-’, (W)ₙ=‘O’, n=1, (V)ₘ=‘CH₂’, m=1, R¹⁹ and R²⁰=H and U=4-tert-butylphenyl. To those skilled in the practices of organic synthesis, 4-tert-butylbenzyl bromide may be replaced by any reasonable Ar—CR¹⁹R²⁰-halide, providing many variations of carboxylic acid (58) under the conditions shown. Thus analogues of (59) exploring a wide range of (V)ₘ and U in general formula (I) may be prepared through the conditions detailed in Scheme 16.

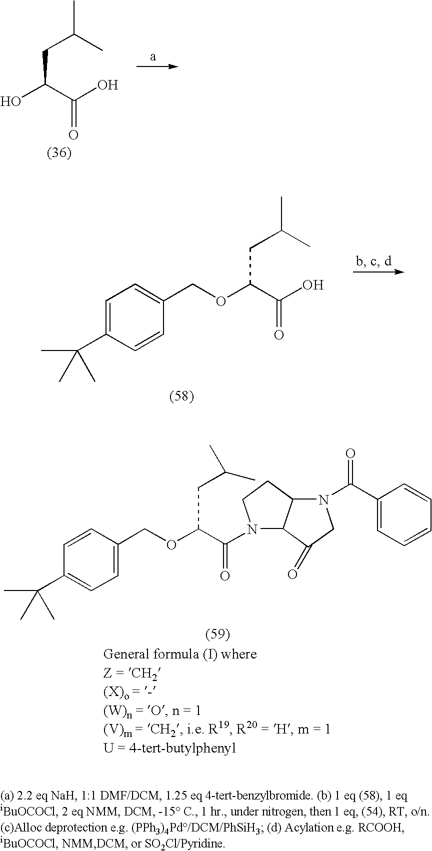

A third strategy for the synthesis of compounds of general formula (I) where the addition of U-V-W-X-Y to the protected building block involves multistep organic reactions comprises:—
(a) Preparation of an appropriately functionalised and protected hexahydropyrrolo[3,2-b]pyrrol-3-one, hexahydropyrrolo[3,2-c]pyrazol-6-one or hexahydro-2-oxa-1,4-diazapentalen-6-one building block in solution.
Preferred protecting groups for solution phase chemistry are the 9-fluorenylmethoxycarbonyl, Nα-tert-butoxycarbonyl, Nα-benzyloxy carbonyl and Nα-allyloxycarbonyl group.
(b) Protection of the ketone functionality of the hexahydropyrrolo[3,2-b]pyrrol-3-one, hexahydropyrrolo[3,2-c]pyrazol-6-one or hexahydro-2-oxa-1,4-diaza pentalen-6-one building block e.g. as a dimethylacetal. Alternatively, the reduced ketone (achiral secondary alcohols (13), (20) and (25)) intermediates may be used and re-oxidised as the final synthetic step.
(c) Standard organic chemistry methods for the conversion of building block (b) towards compounds of general formula (I).

Intermediates may be prepared in solution, followed by coupling to building block (b) and further derivitisation towards compounds of general formula (I) (see Scheme 17 exemplified by preparation and use of the 3-Hydroxy-hexahydro-pyrrolo[3,2-b]pyrrole-1-carboxylic acid allyl ester (61)).

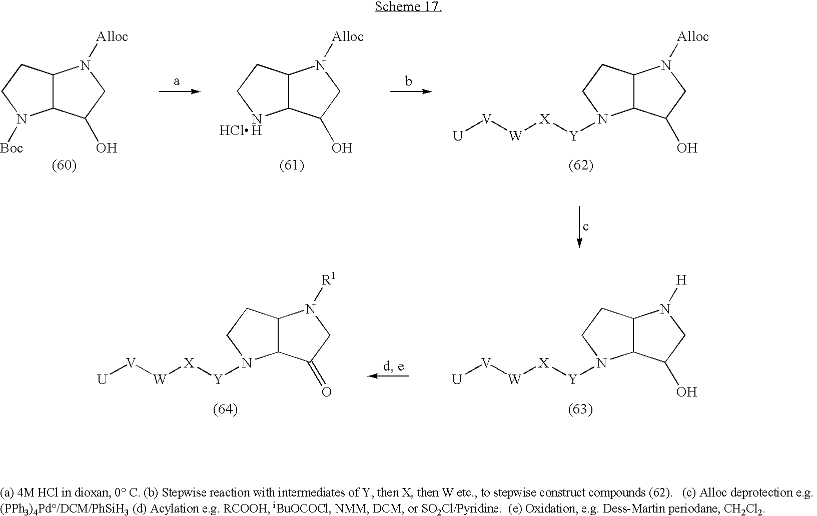

Alternatively, depending upon the types of chemistry used to construct the left hand side U-V-W-X-Y of compounds of general formula (I), the ketone may require protection e.g. as the dimethyl acetal. Such a method is detailed and exemplified in Scheme 18 by the preparation and use of 3,3-Dimethoxy-hexahydro-pyrrolo[3,2-b]pyrrole-1-carboxylic acid allyl ester (66).

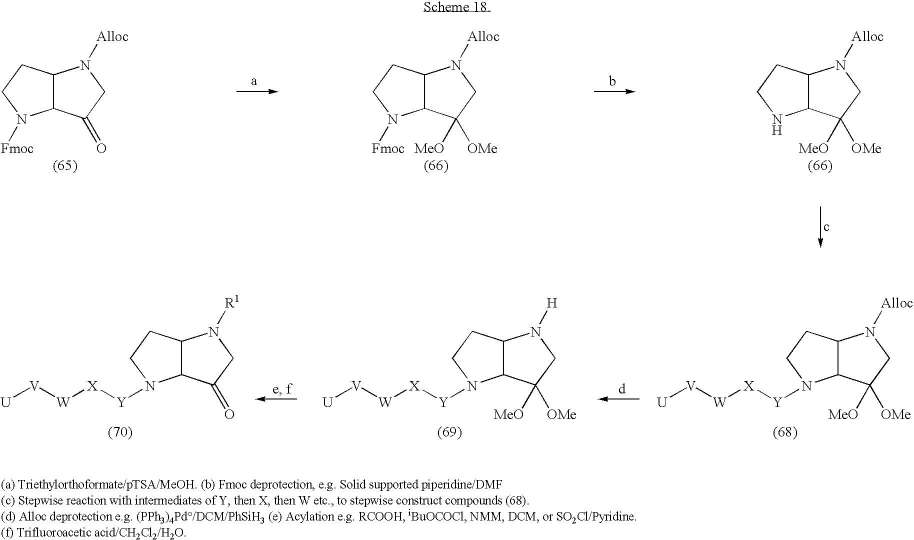

The invention extends to novel intermediates as described above, and to processes for preparing compounds of general formula (I) from each of their immediate precursors. In turn, processes for preparing intermediates from their immediate precursors also form part of the invention.

Compounds of general formula (I) are useful both as laboratory tools and as therapeutic agents. In the laboratory certain compounds of the invention are useful in establishing whether a known or newly discovered cysteine protease contributes a critical or at least significant biochemical function during the establishment or progression of a disease state, a process commonly referred to as ‘target validation’.

According to a second aspect of the invention, there is provided a method of validating a known or putative cysteine protease inhibitor as a therapeutic target, the method comprising:

(a) assessing the in vitro binding of a compound as described above to an isolated known or putative cysteine protease, providing a measure of potency, and optionally, one or more of the steps of:

(b) assessing the binding of the compound to closely related homologous proteases of the target and general house-keeping proteases (e.g. trypsin) to provides a measure of selectivity,

(c) monitoring a cell-based functional marker of a particular cysteine protease activity, in the presence of the compound; and

(d) monitoring an animal model-based functional marker of a particular cysteine protease activity in the presence of the compound.

The invention therefore provides a method of validating a known or putative cysteine protease inhibitor as a therapeutic target. Differing approaches and levels of complexity are appropriate to the effective inhibition and ‘validation’ of a particular target. In the first instance, the method comprises assessing the in vitro binding of a compound of general formula (I) to an isolated known or putative cysteine protease, providing a measure of ‘potency’. An additional assessment of the binding of a compound of general formula (I) to closely related homologous proteases of the target and general house-keeping proteases (e.g. trypsin) provides a measure of ‘selectivity’. A second level of complexity may be assessed by monitoring a cell-based functional marker of a particular cysteine protease activity, in the presence of a compound of general formula (I). For example, a ‘human osteoclast resorption assay’ has been utilised as a cell-based secondary in vitro testing system for monitoring the activity of cathepsin K and the biochemical effect of protease inhibitors (e.g. see WO-A-9850533). An ‘MHC-II processing—T-cell activation assay’ has been utilised as a cell-based secondary in vitro testing system for monitoring the activity of cathepsin S and the biochemical effect of protease inhibitors (Shi, G-P., et al, Immunity, 10, 197-206, 1999). When investigating viral or bacterial infections such a marker could simply be a functional assessment of viral (e.g. count of mRNA copies) or bacterial loading and assessing the biochemical effect of protease inhibitors. A third level of complexity may be assessed by monitoring an animal model-based functional marker of a particular cysteine protease activity, in the presence of a compound of general formula (I). For example, murine models of Leishmania infection, P. vinckei infection, malaria (inhibition of falcipain) and T. cruzi infection (cruzipain), indicate that inhibition of cysteine proteases that play a key role in pathogen propagation is effective in arresting disease symptoms, ‘validating’ said targets.

The invention therefore extends to the use of a compound of general formula (I) in the validation of a known or putative cysteine protease inhibitor as a therapeutic target.

Compounds of general formula (I) are useful for the in vivo treatment or prevention of diseases in which participation of a cysteine protease is implicated.

According to a third aspect of the invention, there is provided a compound of general formula (I) for use in medicine, especially for preventing or treating diseases in which the disease pathology may be modified by inhibiting a cysteine protease.

According to a fourth aspect of the invention, there is provided the use of a compound of general formula (I) in the preparation of a medicament for preventing or treating diseases in which the disease pathology may be modified by inhibiting a cysteine protease.

Certain cysteine proteases function in the normal physiological process of protein degradation in animals, including humans, e.g. in the degradation of connective tissue. However, elevated levels of these enzymes in the body can result in pathological conditions leading to disease. Thus, cysteine proteases have been implicated in various disease states, including but not limited to, infections by Pneumocystis carinii, Trypsanoma cruzi, Trypsanoma brucei brucei and Crithidia fusiculata; as well as in osteoporosis, autoimmunity, schistosomiasis, malaria, tumour metastasis, metachromatic leukodystrophy, muscular dystrophy, amytrophy, and the like. See WO-A-9404172 and EP-A-0603873 and references cited in both of them. Additionally, a secreted bacterial cysteine protease from S. Aureus called staphylopain has been implicated as a bacterial virulence factor (Potempa, J., et al. J. Biol. Chem, 262(6), 2664-2667, 1998).

The invention is useful in the prevention and/or treatment of each of the disease states mentioned or implied above. The present invention also is useful in a methods of treatment or prevention of diseases caused by pathological levels of cysteine proteases, particularly cysteine proteases of the papain superfamily, which methods comprise administering to an animal, particularly a mammal, most particularly a human, in need thereof a compound of the present invention. The present invention particularly provides methods for treating diseases in which cysteine proteases are implicated, including infections by Pneumocystis carinii, Trypsanoma cruzi, Trypsanoma brucei, Leishmania mexicana, Clostridium histolyticum, Staphylococcus aureus, foot-and-mouth disease virus and Crithidia fusiculata; as well as in osteoporosis, autoimmuunity, schistosomiasis, malaria, tumour metastasis, metachromatic leukodystrophy, muscular dystrophy and amytrophy.

Inhibitors of cathepsin K, particularly cathepsin K-specific compounds, are useful for the treatment of osteoporosis, Paget's disease, gingival diseases such as gingivitis and periodontitis, hypercalaemia of malignancy, metabolic bone disease, diseases involving matrix or cartilage degradation, in particular osteoarthritis and rheumatoid arthritis and neoplastic diseases.

In accordance with this invention, an effective amount of a compound of general formula (I) may be administered to inhibit the protease implicated with a particular condition or disease. Of course, this dosage amount will further be modified according to the type of administration of the compound. For example, to achieve an “effective amount” for acute therapy, parenteral administration of a compound of general formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a cysteine protease.

The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect. Prodrugs of compounds of the present invention may be prepared by any suitable method. For those compounds in which the prodrug moiety is a ketone functionality, specifically ketals and/or hemiacetals, the conversion may be effected in accordance with conventional methods.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

According to a fifth aspect of the invention, there is provided a pharmaceutical or veterinary composition comprising one or more compounds of general formula (I) and a pharmaceutically or veterinarily acceptable carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for rectal, nasal, topical (including buccal and sublingual), vaginal or parenteral (including subcutaneous, intramuscular, intravenous and intradermal) administration, but preferably the formulation is an orally administered formulation. The formulations may conveniently be presented in unit dosage form, e.g. tablets and sustained release capsules, and may be prepared by any methods well known in the art of pharmacy.

Such methods include the step of bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

Formulations for oral administration in the present invention may be presented as: discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

According to a sixth aspect of the invention, there is provided a process for the preparation of a pharmaceutical or veterinary composition as described above, the process comprising bringing the active compound(s) into association with the carrier, for example by admixture.

Preferred features for each aspect of the invention are as for each other aspect mutatis mutandis.

### EXPERIMENTAL PROCEDURES

Solution Phase Chemistry—General Methods

All solvents were purchased from ROMIL Ltd (Waterbeach, Cambridge, UK) at SpS or Hi-Dry grade unless otherwise stated. General peptide synthesis reagents were obtained from Chem-Impex Intl. Inc. (Wood Dale Ill. 60191. USA). Thin layer chromatography (TLC) was performed on pre-coated plates (Merck aluminium sheets silica 60 F254, part no. 5554). Visualisation of compounds was achieved under ultraviolet light (254 nm) or by using an appropriate staining reagent. Flash column purification was performed on silica gel 60 (Merck 9385) or Isolute Flash silica cartridge. All analytical HPLC were obtained on Phenomenex Jupiter C₄, 5μ, 300 A, 250×4.6 mm, using mixtures of solvent A=0.1% aq trifluoroacetic acid (TFA) and solvent B=90% acetonitrile/10% solvent A on automated Agilent systems with 215 and 1 or 254 nm UV detection. Unless otherwise stated a gradient of 10-90% B in A over 25 minutes at 1.5 mL/min was performed for full analytical HPLC analysis. HPLC-MS analysis was performed on an Agilent 1100 series LC/MSD, using-automated Agilent HPLC systems, with a gradient of 10-90% B in A over 10 minutes on Phenomenex Columbus C₈, 5μ, 300 A, 50×2.0 mm at 0.4 mL/min. Nuclear magnetic resonance (NMR) were obtained on a Bruker DPX400 (400 MHz 1H frequency, QXI probe) or Bruker DPX500 (500 MHz 1H frequency) in the solvents and temperature indicated (298 K unless otherwise stated). Chemical shifts are expressed in parts per million (δ) and are referenced to residual signals of the solvent. Coupling constants (J) are expressed in Hz. High resolution mass spectrometry was performed on a Micromass QTOF 1.

Solid Phase Chemistry—General Methods

Example inhibitors were prepared through a combination of solution and solid phase Fmoc-based chemistries (see ‘Solid Phase Peptide Synthesis’, Atherton, E. and Sheppard, R. C., IRL Press Ltd, Oxford, UK, 1989, for a general description). An appropriately protected and functionalised building block was prepared in solution (e.g. general compound (6), Scheme 6), then reversibly attached to the solid phase through an appropriate linker followed by rounds of coupling/deprotection/chemical modification (Scheme 6). Example inhibitors were then released (cleaved) from the solid phase, analysed, purified and assayed for inhibition verses a range of proteases.

Generally, multipins (polyamide 1.3→10 μmole loadings, see www.mimotopes.com) were used for the solid phase synthesis, although any suitable solid phase surface could be chosen. In general, the 1.3 μmole gears were used to provide small scale crude examples for preliminary screening, whilst the 10 μmole crowns were used for scale-up synthesis and purification of preferred examples. Standard coupling and Fmoc deprotection methods were employed (see Grabowska, U. et al, J. Comb. Chem. 2(5), 475-490, 2000. for a thorough description of solid phase multipin methodologies).

Preparation of Initial Assembly

Building Block-linker constructs (e.g. (27), typically 10 mg to 100 mg) were carboxyl activated with 2-(1H-benzotriazole-1-yl)-1,1,3,3-tetramethyluronium hexafluoro phosphate (HBTU, 1 mole equivalent), 1-hydroxybenzotriazole.hydrate (HOBT, 1 mole equivalent) and N-methylmorpholine (NMM, 2 mole equivalents) in dimethylformamide (DMF, typically 1 to 10 mL) for 5 minutes. Amino functionalised DA/MDA crowns or HEMA gears (10 μmole per crown/1.2 μmole per gear, 0.33 mole equivalent of total surface amino functionalisation compared to activated construct) were added, followed by additional DMF to cover the solid phase surface. The loading reaction was left overnight. Following overnight loading, crowns/gears were taken through standard cycles washing, Fmoc deprotection and loading quantification (see Grabowska, U. et al) to provide loaded Building Block-linker constructs (e.g. (28)).

Coupling Cycles

The coupling of standard Fmoc-aminoacids (10 or 20 mole equivalent) were performed via carboxyl activated with 2-(1H-benzotriazole-1-yl)-1,1,3,3-tetramethyluronium hexafluoro phosphate (HBTU, 10 or 20 mole equivalent), 1-hydroxybenzotriazole.hydrate (HOBT, 10 or 20 mole equivalent) and N-methylmorpholine (NMM, 20 or 40 mole equivalents) in dimethylformamide, with pre-activation for 5 minutes. Activated species were dispensed to the appropriate wells of a polypropylene 96-well plate (Beckman, 1 mL wells, 500 μL solution per well for crowns or 250 μL solution per well for gears) in a pattern required for synthesis. Loaded free amino Building Block-linker constructs (e.g. (28)) were added and the coupling reaction left overnight. Following overnight coupling, crowns/gears were taken through standard cycles washing and Fmoc deprotection (see Grabowska, U. et al). Identical activation and coupling conditions were used for the coupling of a range of carboxylic acids (R—COOH). Alternatively, chloroformates e.g. morpholine-4-carbonylchloride (10 mole equivalent), were coupled in DMF with the addition of NMM (10 mole equivalents).

Acidolytic Cleavage Cycle

A mixture of 95% TFA/5% water was pre-dispensed into two polystyrene 96-well plates (Beckman, 1 mL wells, 600 μL solution per well for crowns or 300 μL solution per well for gears) in a pattern corresponding to that of the synthesis. The completed multipin assembly was added to the first plate (mother plate), the block covered in tin foil and cleaved for 2 hours. The cleaved multipin assembly was then removed from the first plate and added to the second plate (washing plate) for 15 minutes. The spent multipin assembly was then discarded and the mother 1 washing plates evaporated on an HT4 GeneVac plate evaporator.

Analysis and Purification of Cleaved Examples

(a) Ex 1.2 μmole Gears. 100 μL dimethylsulphoxide (DMSO) was added to each post cleaved and dried washing plate well, thoroughly mixed, transferred to the corresponding post cleaved and dried mother plate well and again thoroughly mixed. 10 μL of this DMSO solution was diluted to 100 μL with a 90% acetonitrile/10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 nm UV analysis). This provided an approximately 10 mM DMSO stock solution of good quality crude examples for preliminary protease inhibitory screening.
(b) Ex 10 μmole Crowns. 500 μL of a 90% acetonitrile/10% 0.1% aq TFA mixture was added to each washing plate well, thoroughly mixed, transferred to the corresponding mother plate well and again thoroughly mixed. 5 μL of this solution was diluted to 100 μL with a 90% acetonitrile/10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 nm WV analysis). The polystyrene blocks containing crude examples were then lyophilised.
(c) Individual examples (ex (b)) were re-dissolved in a 1:1 mixture of 0.1% aq TFA/acetonitrile (1 mL) and purified by semi-preparative HPLC (Phenomenex Jupiter C₄, 5μ, 300 A, 250×10 mm, a 25-90% B in A gradient over 25 mins, 4.0 mL/min, 215 nm UV detection). Fractions were lyophilised into pre-tarred glass sample vials to provide purified examples (typically 2 to 4 mg, 40 to 80% yield).
(d) Purified examples were dissolved in an appropriate volume of DMSO to provide a 10 mM stock solution, for accurate protease inhibitory screening.

EXAMPLES 1-248 were prepared using the general solid phase descriptions above and are inhibitors of cathepsin K with Ki ranging from 1-5000 nM;

### EXAMPLE 1

### (3aR,6aS)-N-[(1S)-1-(4-Benzoyl-6-oxo-hexahydro-pyrrolo[3,2-b]pyrrole-1-carbonyl)-3-methyl-butyl]-4-tert-butylbenzamide

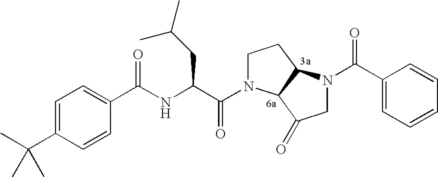

Following the general details from Scheme 1, the required bicycle building block (3aS,6aR) 3-Oxo-hexahydro-pyrrolo[3,2-b]pyrrole-1,4-dicarboxylic acid 1-tert-butyl ester 4-(9H-fluoren-9-ylmethyl) ester (6) was prepared in 8 steps as follows;
(1) Preparation of (2S,3S) 3-hydroxypyrrolidine-1,2-dicarboxylic acid 1-(9H-fluoren-9-ylmethyl) ester.
Trans-3-hydroxy-L-proline (10.0 g, 76.3 mmole) was added to a vigorously stirred, ice-cooled solution of sodium carbonate (16.90 g, 160.2 mmole) in water (100 mL). 1,4-Dioxan (75 mL) was added providing an opaque but mobile mixture. 9-Fluorenylmethyl chloroformate (20.31 g, 80 mmole) in 1,4-dioxan (75 mL) was added over 1 hr, then the ice-cooling removed and the mixture stirred at RT for an additional 2 hr. Additional water (300 mL) was added, the reaction mixture washed with chloroform (2×250 mL) and the combined organic layers discarded. The aqueous phase was acidified with 1N HCl to ˜pH 2, providing a thick opaque mixture. The acidified aqueous mixture was extracted with chloroform (2×500 mL) and the now clear aqueous phase discarded. The opaque combined chloroform layers were dried (Na₂SO₄), filtered and reduced in vacuo to provide batch 1 (5.70 g). The residual precipitate (a mixture of product and drying agent) was triturated with hot methanol (2×250 mL) and the combined methanol solutions reduced in vacuo to provide batch 2 (10.25 g). Batch 1 and 2 were individually analysed by TLC (single UV spot, Rf=0.15, 20% MeOH in CHCl₃), and HPLC-MS (single main UV peak with Rt=7.069 mins, 354.2 [M+H]⁺, 376.2 [M+Na]⁺) and found to be identical, giving a combined yield of 15.95 g (45.2 mmole, 59.2%). Analysis by ¹H and ¹³C NMR showed the presence of cis and trans geometrical isomers around the 3° amide bond.

δ_{H} (DMSO-d₆ at 298 K); 1.80-2.02 (2H_{γ}, m), 3.49-3.62 (2H_{δ}, m), 4.12-4.38 (H_{α}, H_{β}, Fmoc H-9 and CH₂, m), 5.55/5.62 (OH), 7.30-7.31 (2H aromatic, Fmoc H-2 and H-7), 7.35-7.37 (2H aromatic, Fmoc H-3 and H-6), 7.43-7.45 (2H aromatic, Fmoc H-1 and H-8), 7.63-7.65 (2H aromatic, Fmoc H-4 and H-5), 12.8-13.0 (COOH); δ_{C} (DMSO d₆ at 298 K); 31.70/32.70 (d, C_{γ}), 44.68/45.32 (d, C_{δ}), 46.94/46.97 (u, Fmoc C-9), 67.04/67.33 (d, Fmoc CH₂), 68.24/68.51 (u, C_{α}), 73.12/74.23 (u, C_{β}), 120.49/120.52 (u, Fmoc C-4 and C-5), 125.49/125.58 (u, Fmoc C-1 and C-8), 127.50 (u, Fmoc C-2 and C-7), 128.04 (u, Fmoc C-3 and C-6), 140.99/141.09 (q, Fmoc C-4′ and C-5′), 144.02/144.16 (q, Fmoc C-1′ and C-8′), 154.33/154.54 (q, OCON), 172.10/172.39 (COOH).
(2) Preparation of (2S,3R) 3-hydroxypyrrolidine-1,2-dicarboxylic acid 2-allyl ester 1-(9H-fluoren-9-ylmethyl) ester.
(2S,3S) (3-hydroxy)pyrrolidine-1,2-dicarboxylic acid 1-(9H-fluoren-9-ylmethyl) ester (10.9 g, 30.8 mmole) was dissolved in toluene (75 mL) in a Dean-Stark apparatus. Allyl alcohol (20 mL) was added followed by p-toluenesulphonic acid.hydrate (6.05 g, 31.4 mmole). The mixture was refluxed for 1 hr, cooled and CHCl₃ (300 mL) added. The organic layer was washed with NaHCO₃ (300 mL), 0.1N HCl (300 mL) and brine (300 mL), then dried (Na₂SO₄). Filtration and reduction in vacuo gave a pale yellow foam (13.5 g). The crude foam was purified over silica gel (150 g) eluting with a gradient of heptane:ethyl acetate 3:1→1:1. Desired fractions were combined and reduced in vacuo to a colourless gum yield 10.34 g (26.3 mmole, 85.4%). TLC (single UV spot, Rf=0.30, heptane:ethyl acetate 1:1), analytical HPLC Rt=18.849 mins, HPLC-MS (single main UV peak with Rt=8.354 mins, 394.2 [M+H]⁺, 416.2 [M+Na]⁺).

Analysis by ¹H and ¹³C NMR showed the presence of cis and trans geometrical isomers around the 3° amide bond.

δ_{H} (CDCl₃ at 298 K); 2.00-2.21 (2H_{γ}, m), 2.70/2.85 (OH, b), 3.72-3.81 (2H_{δ}, m), 4.12-4.67 (H_{α}, H_{β}, Fmoc H-9 and CH₂, 2×COOCH₂CH═CH₂, m), 5.20-5.40 (2×COOCH₂CH═CH₂, m), 5.82-5.99 (1×COOCH₂CH═CH₂, m), 7.28-7.33 (2H aromatic, Fmoc H-2 and H-7), 7.34-7.41 (2H aromatic, Fmoc H-3 and H-6), 7.53-7.66 (2H aromatic, Fmoc H-1 and H-8), 7.77-7.81 (2H aromatic, Fmoc H-4 and H-5); δ_{C} (CDCl₃ at 298 K); 32.28/33.04 (d, C_{γ}), 44.98/45.32 (d, C_{δ}), 47.56/47.63 (u, Fmoc C-9), 66.44 (d, COOCH₂CH═CH₂), 68.01/68.11 (d, Fmoc CH₂), 68.32/68.72 (u, C_{α}), 74.49/75.67 (u, C_{β}), 119.20/119.48 (d, COOCH₂CH═CH₂), 120.34/120.37 (u, Fmoc C-4 and C-5), 125.36/125.60 (u, Fmoc C-1 and C-8), 127.47 (u, Fmoc C-2 and C-7), 128.06/128.12 (u, Fmoc C-3 and C-6), 131.79/131.94 (u, COOCH₂CH═CH₂), 141.65/141.71 (q, Fmoc C-4′ and C-5′), 144.12/144.34 (q, Fmoc C-1′ and C-8′), 155.13/155.59 (q, OCON), 170.53/170.55 (COOCH₂CH═CH₂).
(3) Preparation of (2S,3R) 3-azidopyrrolidine-1,2-dicarboxylic acid 2-allyl ester 1-(9H-fluoren-9-ylmethyl) ester.
Diethyl azodicarboxylate (1.24 ml, 7.9 mmol) was added dropwise over 20 minutes to a stirred solution of triphenylphosphine (2.07 g, 7.9 mmol) in tetrahydrofuran (30 ml) at 0° C. The mixture was stirred for 5 minutes at 0° C. then a solution of (2S,3S)-3-hydroxypyrrolidine-1,2-dicarboxylic acid 2-allyl ester 1-(9H-fluoren-9-ylmethyl) ester (2.59 g, 6.6 mmol) and hydrazoic acid (14.3 ml of 0.7M solution in toluene) in tetrahydrofuran (30 ml) was added dropwise over 35 minutes. The mixture was stirred for 5 minutes at 0° C. then at ambient temperature for 14 hours. The solvent was removed in vacuo and the residue purified by flash chromatography over silica gel eluting with a gradient of heptane:ethyl acetate 5:1→3:1. Appropriate fractions were combined and the solvents removed in vacuo to obtain (2S,3R) 3-azidopyrrolidine-1,2-dicarboxylic acid allyl ester 1-(9H-fluoren-9-ylmethyl) ester as a colourless oil (1.45 g, 53%). TLC (single UV spot, Rf=0.30, heptane:ethyl acetate 3:1), analytical HPLC main UV peak with Rt=19.896 mins and HPLC-MS 419.2 [M+H]⁺, 441.2 [M+Na]⁺.

δ_{H} (CDCl₃ at 298 K); 2.08-2.25 (2H, H-4, m), 3.52-3.59 (1H, H-5, m), 3.68-3.76 (1H, H-5, m), 4.15 (0.5H, Fmoc-CH₂, t, J=6.6 Hz), 4.24 (0.5H, Fmoc-CH₂, t, J=7.1 Hz), 4.33-4.38 (2H, H-3 and Fmoc-CH, m), 4.44-4.48 (1.5H, 0.5H-2 and Fmoc-CH, m), 4.51-4.66 (1.5H, 0.5H-2 and CH₂CH═CH₂, m), 4.67-4.70 (1H, CH2CH═CH₂, m), 5.21-5.40 (2H, CH₂CH═CH₂, m), 5.84-5.98 (1H, CH₂CH═CH₂, m), 7.26-7.32 (2H, aromatic, Fmoc H-2 and H-7), 7.37-7.40 (2H, aromatic, Fmoc H-3 and H-6), 7.51-7.60 (2H, aromatic, Fmoc H-1 and H-8), 7.74-7.77 (2H, aromatic, Fmoc H-4 and H-5); δ_{C} (CDCl₃ at 298 K); 29.14/30.13 (d, C-5), 44.40/44.72 (d, C-5), 47.12/47.21 (u, Fmoc-CH), 61.02/61.87 (u, C-3), 61.63/62.07 (u, C-2), 66.17 (d, Fmoc-CH₂), 67.65 (d, CH₂CH═CH₂), 118.86/119.11 (d, CH₂CH═CH₂), 119.94/124.83/124.95/125.05/127.03/127.69 (u, aromatic, Fmoc-CH), 131.384/131.50 (u, CH₂CH═CH₂), 141.29 (q, aromatic Fmoc quaternary carbon b), 143.49/143.65/143.92 (q, aromatic Fmoc quaternary carbon a), 154.07/154.49 (q, Fmoc-CO), 168.62/168.70 (q, allyl-CO).
(4) Preparation of (2S,3R) 3-azidopyrrolidine-1,2-dicarboxylic acid 1-(9H-fluoren-9-ylmethyl) ester.
Dichloromethane (30 ml) then phenyltrihydrosilane (0.81 ml, 6.6 mmol) were added consecutively to a stirred mixture of tetrakistriphenylphosphine palladium(0) (76 mg, 0.066 mmol) and (2S,3R)-3-azidopyrrolidine-1,2-dicarboxylic acid allyl ester 1-(9H-fluoren-9-ylmethyl) ester (1.38 g, 3.3 mmol) under argon. The mixture was stirred for 30 minutes then diluted with chloroform (200 ml) and washed with 0.01M hydrochloric acid (200 ml). The aqueous layer was extracted with chloroform (100 ml), then the combined chloroform layers were dried (Na₂SO₄) and the solvent removed in vacuo. The brown residue was purified by flash chromatography over silica gel eluting with a gradient of heptane:ethyl acetate 3.5:1→0:1 followed by methanol:dichloromethane 1:4. Appropriate fractions were combined and the solvents removed in vacuo to leave (2S,3R) 3-azidopyrrolidine-1,2-dicarboxylic acid 1-(9H-fluoren-9-ylmethyl) ester as a brown foam (890 mg, 71%). TLC (main UV spot, Rf=0.20, methanol:chloroform 1:9), analytical HPLC main UV peak with Rt=16.528 mins and HPLC-MS 379.2 [M+H]⁺, 401.1 [M+Na]⁺, 779.3 [2M+Na]⁺.
(5) Preparation of (2S,3R) 3-aminopyrrolidine-1,2-dicarboxylic acid 1-(9H-fluoren-9-ylmethyl) ester.
Acetic acid was added to a suspension of (2S,3R) 3-azidopyrrolidine-1,2-dicarboxylic acid 1-(9H-fluoren-9-ylmethyl) ester (3.25 g, 8.6 mmol), palladium on carbon (10%, 320 mg) and ethanol (80 ml) under an atmosphere of argon. The mixture was then stirred under an atmosphere of hydrogen for 3.5 hours then the hydrogen was replaced with argon and the suspension stored at 0° C. for 14 hours. A further portion of palladium on carbon (10%, 150 mg) was added then the mixture stirred at ambient temperature for 3 hours under an atmosphere of hydrogen. The catalyst was removed by filtration in vacuo through a pad of celite which was washed with acetic acid:water (1:1, 150 ml). The filtrate was concentrated in vacuo then toluene (50 ml) was added to the residue and solvents removed in vacuo. A further portion of toluene was added (50 ml) and the solvent removed in vacuo to leave an oily residue which was triturated with diethyl ether (125 ml) to obtain (2S,3R) 3-aminopyrrolidine-1,2-dicarboxylic acid 1-(9H-fluoren-9-ylmethyl) ester acetate as a pale brown solid (1.05 g, 30%). Analytical HPLC single UV peak with Rt=12.541 mins and HPLC-MS 353.2 [M+H]⁺, 705.3 [2M+Na]⁺.
(6) Preparation of (2S,3R) 3-tert-Butoxycarbonylamino-pyrrolidine-1,2-dicarboxylic acid 1-(9H-fluoren-9-ylmethyl) ester
A solution of di-tert-butyl dicarbonate (210 mg, 0.96 mmol) in 1,4-dioxan (10 ml) was added to a stirred suspension of (2S,3R)-3-aminopyrrolidine-1,2-dicarboxylic acid 1-(9H-fluoren-9-ylmethyl) ester (360 mg, 0.87 mmol) and sodium carbonate (195 mg, 1.84 mmol) in water (10 ml) and 1,4-dioxan (10 ml) over 1 hour at 0° C. The reaction mixture was stirred for 16 hours at ambient temperature then the majority of solvents were removed in vacuo. The residue was dissolved in dichloromethane (200 ml) and water (100 ml) then acidified to pH˜2.5 using 1M hydrochloric acid. The dichloromethane layer was separated then the aqueous layer extracted with dichloromethane. The combined dichloromethane layers were dried (Na₂SO₄) and the solvent removed in vacuo. The orange-brown residue was purified by flash chromatography over silica gel eluting with a gradient of dichloromethane:methanol 19:1→9:1. Appropriate fractions were combined and the solvents removed in vacuo to leave (2S,3R) 3-tert-butoxycarbonylaminopyrrolidine-1,2-dicarboxylic acid 1-(9H-fluoren-9-ylmethyl) ester as a light brown solid (235 mg, 60%). TLC (single UV spot, Rf=0.25, methanol:chloroform 1:9), analytical HPLC single UV peak with Rt=17.476 mins and HPLC-MS 397.2 [M−Bu+2H]⁺, 475.2 [M+Na]⁺, 927.4 [2M+Na]⁺.

δ_{H} (CDCl₃ at 298 K); 1.35 (2H, brs Me₃C), 1.48 (1H, brs Me₃C), 1.75-2.20 (2H, m, H-4), 2.75-3.85 (4H, m, H-5, H-3, Fmoc-CH), 3.85-4.60 (4H, m, Fmoc-CH₂, H-2 and NH), 6.20-6.75 (0.5H, brs, NH), 7.05-7.90 (8H, aromatic); δ_{C} (d₆-DMSO at 298 K); 1.39 and 1.46 (9H total, each s, Me₃C), 1.70-1.85 (1H, m, H-4), 1.70-1.85 (1H, m, H-4), 3.24-3.35 (1H, m, H-5), 3.44-3.54 (1H, m, H-5), 4.02-4.30 (5H, m, H-2, H-3, Fmoc-CH₂ and Fmoc-CH), 6.80 and 7.0 (1H total, each brs, NH), 7.30-7.98 (8H, aromatic)
(7) Preparation of (2S,3R) 3-tert-Butoxycarbonylamino-2-(2-diazo-acetyl)-pyrrolidine-1-carboxylic acid 9H-fluoren-9-ylmethyl ester.
A solution of iso-butyl chloroformate (68 μl, 0.52 mmol) in dichloromethane (2 ml) and a solution of 4-methylmorpholine (105 μl, 0.95 mmol) in dichloromethane (2 ml) were simultaneously added in portions to a stirred suspension of (2S,3R)-3-tert-butoxycarbonylaminopyrrolidine-1,2-dicarboxylic acid 1-(9H-fluoren-9-ylmethyl) ester (215 mg, 0.48 mmol) in dichloromethane (5 ml) at −15° C. over 20 minutes under an atmosphere of nitrogen. The solution was stirred for 2 hours then additional solutions of iso-butyl chloroformate (15 μl, 0.115 mmol) in dichloromethane (0.5 ml) and 4-methylmorpholine (26 μl, 0.237 mmol) in dichloromethane (0.5 ml) were simultaneously added in one portion. The mixture was stirred for 30 minutes at −15° C. then ethereal diazomethane [˜15 mmol generated from diazald (4.7 g mmol) addition in diethyl ether (75 ml) to sodium hydroxide (5.25 g) in water (7.5 ml)/ethanol (15 ml) at 65° C.] was cautiously added and the resulting yellow solution stirred at room temperature for 16 hrs. Acetic acid (˜1 ml) was cautiously added (until effervescence had ceased), then the mixture was diluted with diethyl ether (100 ml). The ethereal layer was washed with water (3×100 ml), dried (Na₂SO₄) and the solvents removed in vacuo to leave an oily residue (250 mg) which was purified by flash chromatography over silica gel eluting with a gradient of heptane:ethyl acetate 2:1→1:1. Appropriate fractions were combined and the solvents removed in vacuo to leave (2S,3R) 3-tert-butoxycarbonylamino-2-(2-diazoacetyl)pyrrolidine-1-carboxylic acid 9H-fluoren-9-ylmethyl ester as a pale yellow solid (91 mg, 40%). TLC (single UV spot, Rf=0.4, heptane:ethyl acetate 1:1), analytical HPLC main UV peak with Rt=18.363 mins and HPLC-MS 449.2 [M−N₂+H]⁺, 499.2 [M+Na]⁺, 975.5 [2M+Na]⁺.
(8) Cyclisation to (3aS,6aR) 3-Oxo-hexahydro-pyrrolo[3,2-b]pyrrole-1,4-dicarboxylic acid 1-tert-butyl ester 4-(9H-fluoren-9-ylmethyl) ester (6)
A solution of (2S,3R) 3-tert-butoxycarbonylamino-2-(2-diazoacetyl) pyrrolidine-1-carboxylic acid 9H-fluoren-9-ylmethyl ester (100 mg, 0.21 mmol) in chloroform (2.5 ml) was added dropwise over 28 minutes to a stirred suspension of rhodium (II) acetate dimer (10 mg) in toluene (2.5 ml) at 75° C. under an atmosphere of argon. The mixture was stirred for an additional 30 minutes at this temperature then the solvents removed in vacuo to leave an oily residue which was purified by flash chromatography over silica gel eluting with a gradient of hexane:ethyl acetate 3:1→1:1. Appropriate fractions were combined and the solvents removed in vacuo to leave (3aS,6aR) 3-oxo-hexahydropyrrolo[3,2-b]pyrrole-1,4-dicarboxylic acid 1-tert-butyl ester 4-(9H-fluoren-9-ylmethyl) ester as a white solid (28 mg, 30%). TLC (two UV spots, major and minor Rf=0.30 and 0.35 respectively, hexane:ethyl acetate 7:3), analytical HPLC broad group of UV peaks with Rt=20.043-21.472 mins and HPLC-MS 449.2 [M+H]⁺, 471.2 [M+Na]⁺, 919.4 [2M+Na]⁺.

δ_{H} (CDCl₃ at 298 K); (Spectrum poorly resolved) 1.46 (9H, s, Me₃C), 1.85-2.35 (2H, m, H-6), 3.2-5.0 (9H, m, 2×H-2, H-3a, 2×H-5, H-6a, Fmoc-CH₂, Fmoc-CH), 7.2-7.85 (8H, aromatic).

Alternatively, following the general details from Scheme 2, the required bicycle building block (3aS,6aR) 3-Oxo-hexahydro-pyrrolo[3,2-b]pyrrole-1,4-dicarboxylic acid 1-tert-butyl ester 4-(9H-fluoren-9-ylmethyl) ester (6) was prepared following Schemes 19 and 20;

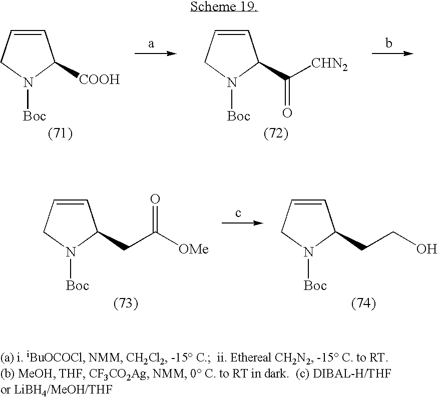

### Preparation of (S)-2-(2-diazoacetyl)-2,5-dihydropyrrole-1-carboxylic acid tert-butyl ester (72)

2,5-Dihydropyrrole-1,2-dicarboxylic acid 1-tert-butyl ester (71) (1.066 g, 5 mmol) was dissolved with stirring in anhydrous dichloromethane (40 ml). The reaction was flushed with nitrogen and cooled to −15° C. iso-Butylchloroformate (0.713 ml, 5.5 mmol) in anhydrous dichloromethane (5 ml) and 4-methylmorpholine (1.099 ml, 10 mmol) in anhydrous dichloromethane (5 ml) were added simultaneously in 1 ml aliquots over 50 minutes. The mixture was stirred for 2.5 hours at −15° C. then ethereal diazomethane [˜15 mmol generated from addition of diazald (4.7 g) in diethyl ether (75 ml) onto sodium hydroxide (5.25 g) in water (7.5 ml)/ethanol (15 ml) at 60° C.] was added to the activated amino acid solution. The mixture was allowed to warm to ambient temperature and stirred for 2.5 hours. A few drops of acetic acid were cautiously added to the mixture, followed by dichloromethane (40 ml). The ethereal layers were washed with aqueous saturated sodium hydrogen carbonate solution (2×40 ml), dried (Na₂SO₄) and the solvents removed in vacuo to leave a yellow residue (1.4 g). Flash chromatography of the residue over silica (35 g) eluting with ethyl acetate:heptane 3:7 gave (S)-2-(2-diazoacetyl)-2,5-dihydropyrrole-1-carboxylic acid tert-butyl ester (72) (1.024 g, 86%). TLC (Single spot, R_{f}=0.47, EtOAc:heptane 1:1), analytical HPLC Rₜ=11.537 min; HPLC-MS 497.2 [2M+Na]⁺; d_{H} (500 MHz, CDCl₃) 1.41-1.51 (9H, m, C(CH₃)₃), 4.11-4.35 (2H, m, BocNCH₂), 4.86-5.05 (1H, m, BocNCH), 5.25-5.50 (1H, m, CHN₂), 5.70-5.80 (1H, m, olefinic CH) and 5.88-6.03 (1H, m, olefinic CH); d_{C} (125 MHz, CDCl₃) 28.3 and 28.4 (C(CH₃)₃), 51.8 and 52.3 (CHN₂), 53.65 and 54.0 (BocNCH₂), 71.5 and 72.3 (BocNCH), 80.6 and 80.9 (OC(CH₃)₃), 126.1 and 126.3 (olefinic CH), 128.35 and 128.5 (olefinic CH), 153.7 and 154.15 (NCO₂), 192.7 and 193.4 (COCHN₂).

### Preparation of (R)-2-methoxycarbonylmethyl-2,5-dihydropyrrole-1-carboxylic acid tert-butyl ester (73)

(S)-2-(2-Diazoacetyl)-2,5-dihydropyrrole-1-carboxylic acid tert-butyl ester (72) (912 mg, 3.85 mmol) was dissolved in tetrahydrofuran (14 ml) and methanol (1.6 ml) then cooled to 0° C. A solution of silver trifluoroacetate (94 mg) in 4-methylmorpholine (1.06 ml) was added, and the mixture allowed to warm to ambient temperature over 6 hours in the dark. Methanol (40 ml) was added, followed by 10% aqueous citric acid solution (100 ml). The majority of the organic solvents were removed in vacuo then the aqueous phase extracted with ethyl acetate (3×40 ml). The combined organic layers were washed with brine (40 ml), dried (Na₂SO₄) and evaporated in vacuo to afford a residue (1.35 g). Flash chromatography of the residue over silica (200 g) eluting with ethyl acetate:hexane 3:17 afforded (R)-2-methoxycarbonylmethyl-2,5-dihydropyrrole-1-carboxylic acid tert-butyl ester (73) as a colourless oil (670 mg, 72%). TLC (Single spot, R_{f}=0.27, EtOAc:hexane 1:4), analytical HPLC Rₜ=15.033 min; HPLC-MS 505.3 [2M+Na]⁺; d_{H} (500 MHz, CDCl₃) 1.44-1.53 (9H, m, C(CH₃)₃), 2.37-2.55 (1H, m, CH₂CO₂Me), 2.90-4.00 (1H, ma, CH₂CO₂Me), 3.63-3.70 (3H, m, OCH₃), 3.97-4.26 (2H, m, BocNCH₂), 4.70-4.90 (1H, m, BocNCH), 5.74-5.89 (2H, m, 2× olefinic CH); d_{C} (125 MHz, CDCl₃) 28.2, 28.3 and 28.5 (C(CH₃)₃), 39.4 and 38.4 (CH₂CO₂Me), 51.5 and 51.6 (OCH₃), 53.3 and 53.5 (BocNCH₂), 60.7 and 60.9 (BocNCH), 79.6 and 80.0 (OC(CH₃)₃), 126.0 and 126.1 (olefinic CH), 129.3 and 129.5 (olefinic CH), 153.9 (NCO₂), 171.5 and 171.7 (CO₂Me).

### Preparation of (R)-2-(2-hydroxyethyl)-2,5-dihydropyrrole-1-carboxylic acid tert-butyl ester (74)

Diisobutylaluminium hydride (1M solution in tetrahydrofuran, 13.6 ml, 13.6 mmol) was added dropwise over 20 minutes to a stirred solution of (R)-2-methoxycarbonylmethyl-2,5-dihydropyrrole-1-carboxylic acid tert-butyl ester (73) (630 mg, 2.61 mmol) in tetrahydrofuran (20 ml) at −78° C. under a nitrogen atmosphere. The mixture was stirred for 2 hours at −78° C. then at ambient temperature for 18 hours. Methanol (11.94 ml) was slowly added to the mixture, followed by ethyl acetate (40 ml) and magnesium sulfate. The resultant slurry was vigorously stirred for 2 hours, then filtered and the solid residue washed with excess ethyl acetate. The filtrate was evaporated in vacuo to afford a residue (1.4 g). Flash chromatography of the residue over silica gel (150 g) eluting with ethyl acetate:hexane 7:13 gave (R)-2-(2-hydroxyethyl)-2,5-dihydropyrrole-1-carboxylic acid tert-butyl ester (74) (430 mg, 77%). TLC (Single spot, R_{f}=0.37, EtOAc:hexane 1:1), analytical HPLC Rₜ=12.161 min; HPLC-MS 236.1 [M+Na]⁺, 449.3 [2M+Na]⁺; [a]_{D}²² −112° (c=1, CHCl₃); d_{H} (500 MHz, CDCl₃) 1.42-1.55 (10H, br. s, C(CH₃)₃ and NCHCH₂), 1.84-1.95 (1H, m, NCHCH₂), 3.60-3.72 (2H, m, CH₂OH), 3.93-4.28 (2H, m, BocNCH₂), 4.53-4.78 (1H, m, BocNCH), 5.67-5.84 (2H, m, 2× olefinic CH); d_{C} (125 MHz, CDCl₃) 28.4 (C(CH₃)₃), 37.4 and 38.7 (CH₂CH₂OH), 53.45 and 53.6 (NCH₂), 59.2 and 59.6 (OCH₂), 61.2 and 61.9 (BocNCH), 79.9 and 80.1 (OC(CH₃)₃), 124.4 and 125.3 (olefinic CH), 130.3 and 131.1 (olefinic CH), 154.4 and 156.0 (NCO₂).

### Alternative preparation of (R)-2-(2-hydroxyethyl)-2,5-dihydropyrrole-1-carboxylic acid tert-butyl ester (74)

Methanol (0.27 ml, 6.7 mmol) was added dropwise to a stirred suspension of lithium borohydride (146 mg, 6.6 mmol) in tetrahydrofuran (3.5 ml) under an atmosphere of argon over 4 minutes, followed by a solution of (R)-2-methoxycarbonylmethyl-2,5-dihydropyrrole-1-carboxylic acid tert-butyl ester (73) (0.8 g, 3.3 mmol) in tetrahydrofuran (8 ml) over 15 minutes. The mixture was stirred for 1 hour then poured into water (25 ml). The product was extracted into dichloromethane (3×20 ml), dried (Na₂SO₄), and the solvents removed in vacuo. The residue was purified by flash chromatography over silica eluting with ethyl acetate:heptane mixtures 0:100 to 25:75 to give (R)-2-(2-hydroxyethyl)-2,5-dihydropyrrole-1-carboxylic acid tert-butyl ester (74) as a colourless oil (0.48 g, 67%), [a]_{D}²² −127° (c=1, CHCl₃).

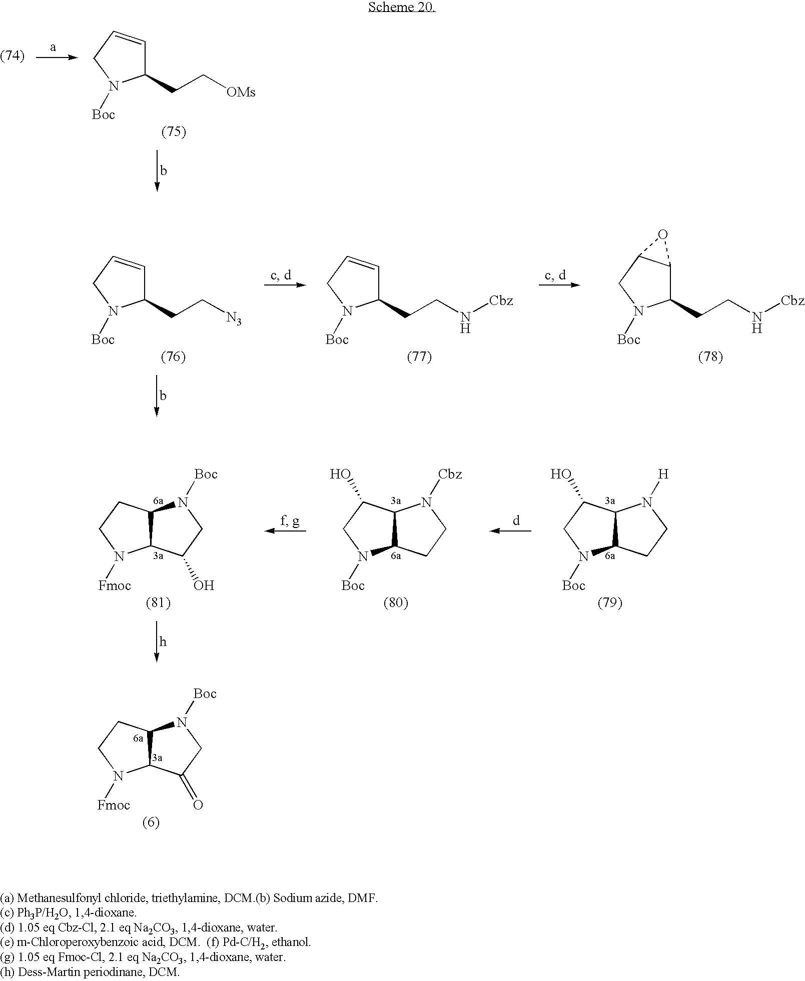

### Preparation of (R)-2-(2-methanesulfonylethyl)-2,5-dihydropyrrole-1-carboxylic acid tert-butyl ester (75)

Triethylamine (2.35 ml, 16.9 mmol) was added dropwise to a stirred solution of (R)-2-(2-hydroxyethyl)-2,5-dihydropyrrole-1-carboxylic acid tert-butyl ester (74) (2.33 g, 10.9 mmol) in dichloromethane (20 ml) at 0° C. over 2 minutes followed by methanesulfonyl chloride (1.27 ml, 16.4 mmol) over 4 minutes. The mixture was stirred for 1 hour at 0° C. then washed with water (170 ml) and brine (170 ml), dried (Na₂SO₄) and the solvents removed in vacuo to leave a residue (3.42 g), which was used without further purification (see below). HPLC-MS 236.0 [M+2H−Bu]⁺, 314.1 [M+Na]⁺, 605.1 [2M+Na]⁺.

### Preparation of (R)-2-(2-azidoethyl)-2,5-dihydropyrrole-1-carboxylic acid tert-butyl ester (76)

Sodium azide (3.55 g, 54.7 mmol) was added to a stirred solution of (R)-2-(2-methanesulfonylethyl)-2,5-dihydropyrrole-1-carboxylic acid tert-butyl ester (75) (prepared as above) in dimethylformamide (45 ml) under an atmosphere of argon. The mixture was stirred at 60° C. for 1.5 hours then the majority of solvents were removed by distillation in vacuo and the residue partitioned between water (200 ml) and ethyl acetate (200 ml). The ethyl acetate layer was washed with brine (150 ml), dried (Na₂SO₄), and the solvents removed in vacuo to leave a residue (2.49 g) which was purified by flash chromatography over silica eluting with ethyl acetate:heptane mixtures 0:100 to 10:90 to give (R)-2-(2-azidoethyl)-2,5-dihydropyrrole-1-carboxylic acid tert-butyl ester (76) as a colourless oil (2.05 g, 79%). TLC (Single spot, R_{f}=0.45, EtOAc:hexane 3:7), analytical HPLC Rₜ=15.910 min; HPLC-MS 139.1 [M+2H−Boc]⁺, 183.1 [M+2H−Bu]⁺, 499.2 [2M+Na]⁺; d_{H} (500 MHz, CDCl₃) 1.40-1.50 (9H, m, C(CH₃)₃), 1.90-2.10 (2H, m, NCHCH₂), 3.17-3.33 (2H, m, CH₂N₃), 3.96-4.27 (2H, m, BocNCH₂), 4.53-4.68 (1H, m, BocNCH), 5.66-5.86 (2H, m, 2× olefinic CH); d_{C} (125 MHz, CDCl₃) 28.3 and 28.5 (C(CH₃)₃), 32.5 and 33.0 (CH₂CH₂N₃), 47.5 and 47.9 (CH₂N₃), 53.6 and 53.8 (BocNCH₂), 62.0 and 62.3 (BocNCH), 79.55 and 79.9 (OC(CH₃)₃), 125.6 and 126.1 (olefinic CH), 128.9 and 129.4 (olefinic CH), 154.2 and 154.3 (NCO₂), followed by (R)-2-(2-hydroxyethyl)-2,5-dihydropyrrole-1-carboxylic acid tert-butyl ester (74) (22 mg, 9%).

### Preparation of (R)-2-(2-benzyloxycarbonylaminoethyl)-2,5-dihydropyrrole-1-carboxylic acid tert-butyl ester (77)

Water (2.1 ml, 118 mmol) was added to a stirred solution of (R)-2-(2-azidoethyl)-2,5-dihydropyrrole-1-carboxylic acid tert-butyl ester (76) (2.8 g, 11.8 mmol) and triphenylphosphine (4.6 g, 17.5 mmol) in tetrahydrofuran (350 ml) under an atmosphere of argon. The solution was heated at 45° C. for 7.5 hours then at ambient temperature for 14 hours. An aliquot (18.5 ml, ˜0.63 mmol) was removed, concentrated in vacuo then azeotroped with toluene (3×10 ml) and used for the preparation of (2R)-2-[2-((2S)-2-benzyloxycarbonylamino-4-methylpentanoylamino) ethyl]-2,5-dihydropyrrole-1-carboxylic acid tert-butyl ester (140) (see Scheme 28). An additional 5.0 ml aliquot was removed for analysis, then the remainder of the solution was concentrated in vacuo to obtain an oily residue. The residue was dissolved in 1,4-dioxane (35 ml) with stirring, ice-cooled and a solution of sodium carbonate (2.45 g, 23.1 mmol) in water (35 ml) was added. Benzyl chloroformate (2.18 g, 1.824 ml, 12.8 mmol) in 1,4-dioxane (10 ml) was then added dropwise over 30 minutes and the mixture stirred for an additional 30 minutes before adding water (250 ml). The aqueous phase was extracted with dichloromethane (2×250 ml) and the combined organic layers were dried (Na₂SO₄), filtered and reduced in vacuo to leave a clear mobile oil (10.2 g). Flash chromatography over silica, eluting with ethyl acetate:heptane mixtures gave (R)-2-(2-benzyloxycarbonylaminoethyl)-2,5-dihydropyrrole-1-carboxylic acid tert-butyl ester (77) (3.58 g, 94%) as a mobile colourless oil. TLC (R_{f}=0.32, EtOAc:heptane 1:1), analytical HPLC single main peak, Rₜ=17.39 min., HPLC-MS 247.1 [M+2H−Boc]⁺, 291.1 [M+2H−Bu]⁺, 347.1 [M+H]⁺, 369.1 [M+Na]⁺, 715.2 [2M+Na]⁺; Elemental analysis C₁₉H₂₆N₂O₄ req. (fnd.) % C 65.87 (65.79), % H 7.56 (7.53), % N 8.09 (7.97); HRMS C₁₉H₂₆N₂O₄Na req. 369.1790, fnd. 369.1803 (3.37 ppm); δ_{H} (500 z, CDCl₃) 1.45 (9H, br. s, C(CH₃)₃), 1.60-1.95 (2H, m, BocNCHCH₂), 3.00-3.44 (2H, m, CH₂NH), 3.90-4.29 (2H, m, BocNCH₂), 4.45-4.81 (1H, m, BocNCH), 5.01-5.16 (2H, m, OCH₂Ph), 5.50-5.83 (2H, m, 2× olefinic CH) and 7.25-7.38 (6H, m, C₆H₅ and NH); δ_{C} (125 MHz, CDCl₃) 28.4 (C(CH₃)₃), 34.4, 34.6 (CH₂CH₂NH), 37.2, 37.6 (CH₂NH), 53.6, 53.7 (BocNCH₂), 61.7, 62.1 (BocNCH), 66.4, 66.6 (OCH₂Ph), 79.6, 79.9 (OC(CH₃)₃), 125.2, 125.9, 127.0, 127.6, 127.9, 128.0, 128.4, 129.5, 130.2 (5× aromatic CH and 2× olefinic CH), 154.3, 155.0, 156.2, 156.5 (NHCO₂ and NCO₂).

### Preparation of (2R)-2-(2-benzyloxycarbonylaminoethyl)-6-oxa-3-aza-bicyclo [3.1.0]hexane-3-carboxylic acid tert-butyl ester (78)

(R)-2-(2-Benzyloxycarbonylaminoethyl)-2,5-dihydropyrrole-1-carboxylic acid tert-butyl ester (77) (3.57 g, 10.3 mmol) was dissolved in anhydrous dichloromethane (60 ml) with stirring and meta-chloroperoxybenzoic acid (27.3 g, 65% reagent, 103 mmol) added. The mixture was stirred at ambient temperature under argon for 16 hours. Dichloromethane (400 ml) was added and the organic phase washed with 10% aqueous w/v solution of sodium hydroxide (2×400 ml), then dried (Na₂SO₄), filtered and reduced in vacuo to leave a clear oil (˜5 g). Flash chromatography over silica, eluting with ethyl acetate:heptane mixtures gave (2R)-2-(2-benzyloxycarbonyl aminoethyl)-6-oxa-3-aza-bicyclo [3.1.0]hexane-3-carboxylic acid-tert-butyl ester (78) (3.57 g, 95.3%) as a mobile colourless oil. TLC (R_{f}=0.36 (minor) and 0.40 (major) (mixture of anti and syn epoxides), EtOAc:heptane 2:1), analytical HPLC single main peak, Rₜ=17.74 min., HPLC-MS 263.1 [M+2H−Boc]⁺, 307.1 [M+2H−Bu]⁺, 363.1 [M+H]⁺, 385.1 [M+Na]⁺, 747.2 [2M+Na]⁺; Elemental analysis C₁₉H₂₆N₂O₅ req. (fnd.) % C 62.97 (62.93), % H 7.23 (7.22), % N 7.73 (7.61); HRMS C₁₉H₂₆N₂O₅Na req. 385.1739, fnd. 385.1725 (−3.82 ppm); d_{H} (500 MHz, CDCl₃) 1.32-1.62 (10H, m, C(CH₃)₃ and CH₂CH₂NH), 1.67-2.00 (1H, m, CH₂CH₂NH), 2.90-4.21 (7H, m, CH₂NH, BocNCHCH, BocNCH₂CH), 4.70-5.17 (2H, m, OCH₂Ph), 5.78-6.05 (1H, m, NH) and 7.27-7.37 (5H, aromatics); d_{C} (125 MHz, CDCl₃) 28.1, 28.3, 28.35 and 28.4 (C(CH₃)₃), 30.8 and 31.2 (CH₂CH₂NH), 37.4 and 37.7 (CH₂NH), 46.15 and 46.6 (BocNCH₂), 53.9, 54.2, 54.9 and 55.8 (2× epoxide CH), 58.1 and 58.2 (BocNCH), 66.5 and 66.7 (OCH₂Ph), 80.3 and 80.7 (OC(CH₃)₃), 128.0, 128.1, 128.2, 128.4, 128.5 (5× aromatic CH), 136.7 (OCH₂C), 155.1, 155.9, 156.3 and 156.6 (NHCO₂ and NCO₂).

### Preparation of (3S,3aS,6aR)-3-hydroxyhexahydropyrrolo[3,2-b]pyrrole-1-carboxylic acid tert-butyl ester (79)

(2R)-2-(2-Benzyloxycarbonylaminoethyl)-6-oxa-3-aza-bicyclo[3.1.0]hexane-3-carboxylic acid tert-butyl ester (78) (3.57 g, 9.86 mmol) was dissolved in ethanol (60 ml), cooled to 0° C. and 10% palladium on charcoal (0.40 g) added. The mixture was stirred, then evacuated and flushed with hydrogen. The mixture was allowed to warm to ambient temperature and after 2.5 hours filtered through celite. The filter cake was washed with ethanol (3×60 ml) and the combined organic filtrates reduced in vacuo to provide crude (3S,3aS,6aR)-3-hydroxyhexahydropyrrolo[3,2-b]pyrrole-1-carboxylic acid tert-butyl ester (79) (˜2.4 g). HPLC-MS 173.1 [M+2H−Bu]⁺, 229.1 [M+H]⁺.

### Preparation of (3S,3aS,6aR)-3-hydroxyhexahydropyrrolo[3,2-b]pyrrole-1,4-dicarboxylic acid 4-benzyl ester 1-tert-butyl ester (80)

Crude (3S,3aS,6aR)-3-hydroxyhexahydropyrrolo[3,2-b]pyrrole-1-carboxylic acid tert-butyl ester (79) (−2.4 g) was dissolved in 1,4-dioxane (30 ml) with stirring, ice-cooled and a solution of sodium carbonate (2.19 g, 20.7 mmol) in water (25 ml) was added. Benzyl chloroformate (1.63 ml, 11.4 mmol) in 1,4-dioxane (15 ml) was then added dropwise over 30 minutes and the mixture stirred for a further 30 minutes. The mixture was then reduced in vacuo by approximately ⅔ volume to leave a mobile pulp. Water (200 ml) was added and the aqueous phase extracted with dichloromethane (2×100 ml). The combined organic layers were dried (Na₂SO₄), filtered and reduced in vacuo to leave a clear mobile oil (3.96 g). Flash chromatography over silica, eluting with ethyl acetate:heptane mixtures gave (3S,3aS,6aR)-3-hydroxyhexahydropyrrolo[3,2-b]pyrrole-1,4-dicarboxylic acid 4-benzyl ester 1-tert-butyl ester (80) (2.16 g, 60.5%) as an opaque gum. TLC (R_{f}=0.15, EtOAc:heptane 1:1), analytical HPLC single main peak, Rₜ=17.15 min., HPLC-MS 263.1 [M+2H−Boc]⁺, 307.1 [M+2H−Bu]⁺, 363.1 [M+H]⁺, 385.1 [M+Na]⁺, 747.2 [2M+Na]⁺; Elemental analysis C₁₉H₂₆N₂O₅ req. (fnd.) % C 62.97 (62.82), % H 7.23 (7.39), % N 7.73 (7.57); HRMS C₁₉H₂₆N₂O₅Na req. 385.1739, fnd. 385.1725 (2.15 ppm); δ_{H} (400 MHz, CD₃CN, T=75° C.) 1.46 (9H, x, C(CH₃)₃), 1.90-2.00 (1H, m obscured by NMR solvent peaks, BocNCHCH₂), 2.14 (1H, dd, J=6.15, 13.15 Hz, BocNCHCH₂), 3.07-3.20 (2H, m, OH+CbzNCH₂), 3.24 (1H, dd, J=3.8, 12.2 Hz, BocNCH₂), 3.51 (1H, d, J=12.2 Hz, BocNCH₂), 3.68 (1H, ddd, J=1.7, 8.6, 10.9 Hz, CbzNCH₂), 4.10 (1H, br. d, J=5.8 Hz, CbzNCH), 4.27 (1H, br. s, CHOH), 4.40-4.46 (1H, m, BocNCR), 5.12 (1H, d, J=12.7 Hz, OCH₂Ph), 5.16 (1H, d, J=12.7 Hz, OCH₂Ph) and 7.42-7.29 (5H, aromatics); δ_{C} (120 MHz, CDCl₃) 28.5 (C(CH₃)₃), 29.7, 30.4, 31.2, 31.9, 32.0 (BocNCHCH₂), 45.5, 45.7 (CbzNCH₂), 53.1, 53.4, 53.5 (BocNCH₂), 60.1, 61.2 (BocNCH), 67.2, 67.6, 68.2, 68.4, 69.0 (OCH₂Ph+Cbz−NCH), 72.7, 73.3, 73.4 (CHOH), 79.9, 80.1 (OC(CH₃)₃), 127.9, 128.0, 128.2, 128.3, 128.5, 128.6, 136.3, 136.4 (aromatics), 154.1, 154.2, 155.2 (2×NCO₂).

### Preparation of (3S,3aS,6aR)-3-hydroxyhexahydropyrrolo[3,2-b]pyrrole-1,4-dicarboxylic acid 1-tert-butyl ester 4-(9H-fluoren-9-ylmethyl) ester (81)

(3S,3aS,6aR)-3-Hydroxyhexahydropyrrolo[3,2-b]pyrrole-1,4-dicarboxylic acid 4-benzyl ester 1-tert-butyl ester (80) (0.54 g, 1.5 mmol) was dissolved in ethanol (10 ml), cooled to 0° C. and 10% palladium on charcoal (0.055 g) added. The mixture was stirred, then evacuated and flushed with hydrogen. The mixture was warmed to ambient temperature and after 2.5 hours filtered through celite. The filter cake was washed with ethanol (3×10 ml) and the combined filtrates reduced in vacuo to provide the crude amine (˜0.36 g). HPLC-MS 173.1 [M+2H−Bu]⁺, 229.1 [M+H]⁺. The crude amine was dissolved in 1,4-dioxane (15 ml) with stirring, ice-cooled and a solution of sodium carbonate (0.33 g, 3.15 mmol) in water (15 ml) was added. 9-Fluorenylmethyl chloroformate (0.463 g, 1.79 mmol) in 1,4-dioxane (10 ml) was added dropwise over 30 minutes and the mixture stirred for a further 30 minutes. Water (200 ml) was then added and the aqueous phase extracted with ethyl acetate (2×100 ml). The combined organic layers were dried (Na₂SO₄), filtered and reduced in vacuo to leave a clear mobile oil (1.02 g). Flash chromatography over silica, eluting with ethyl acetate:heptane mixtures gave (3S,3aS,6aR)-3-hydroxy hexahydropyrrolo[3,2-b]pyrrole-1,4-dicarboxylic acid 1-tert-butyl ester 4-(9H-fluoren-9-ylmethyl) ester (81) (0.64 g, 95%) as a fine white crystalline solid. TLC (R_{f}=0.33, EtOAc:heptane 2:1), analytical HPLC single main peak, Rₜ=19.98 min., HPLC-MS 395.1 [M+2H−Bu]⁺, 451.1 [M+H]⁺, 473.1 [M+Na]⁺, 923.2 [2M+Na]⁺; Elemental analysis C₂₆H₃₀N₂O₅ req. (fnd.) % C 69.31 (69.11), % H 6.71 (7.06), % N 6.22 (5.84); HRMS C₂₆H₃₀N₂O₅Na req. 473.2052, fnd. 473.2053 (0.06 ppm); OH (400 MHz, CD₃CN, T=75° C.) 1.46 (9H, s, C(CH₃)₃), 1.75-1.90 (1H, m, BocNCHCH₂), 2.05-2.13 (1H, m, BocNCHCH₂), 3.02 (1H, m, FmocNCH₂), 3.08-3.20 (1H, m, BocNCH₂), 3.46 (1H, m, BocNCH₂), 3.46-3.60 (1H, m, FmocNCH₂), 3.90-4.15 (2H, m, FmocNCH and CHOH), 4.28 (1H, t, J=6.1 Hz, FmocCH), 4.34-4.40 (1H, m, BocNCH), 4.49 (2H, d, J=6.1 Hz, FmocCH₂), 7.31-7.45 (4H, m, Fmoc aromatics), 7.65 (2H, d, J=7.3 Hz, Fmoc aromatics), 7.83 (2H, d, J=7.5 Hz, Fmoc aromatics); δ_{C} (100 MHz, CDCl₃) 28.45 (C(CH₃)₃), 30.2, 31.2, 32.0 (BocNCHCH₂), 44.8, 45.3, 45.6 (FmocNCH₂), 47.3, 47.4 (FmocCH), 52.8, 53.1, 53.4, 53.5 (BocNCH₂), 60.1, 60.8 (BocNCH), 65.9, 66.2, 67.3 (FmocCH₂), 67.85, 68.4, 68.9 (FmocNCH), 72.5, 72.9, 73.3, 73.6 (CHOH), 79.95 (OC(CH₃)₃), 119.8, 120.0, 124.6, 124.9, 125.0, 127.0, 127.4, 127.8 (Fmoc CH aromatics), 141.3, 141.5, 143.7, 143.8, 144.1 (Fmoc quaternary aromatics), 154.0, 154.3, 155.0, 155.2 (2×NCO₂).

### Alternative preparation of (3S,3aS,6aR)-3-hydroxyhexahydropyrrolo[3,2-b]pyrrole-1,4-dicarboxylic acid 1-tert-butyl ester 4-(9H-fluoren-9-ylmethyl) ester (81)

meta-Chloroperoxybenzoic acid (864 mg, 57-86%) was added to a solution of (R)-2-(2-azidoethyl)-2,5-dihydropyrrole-1-carboxylic acid tert-butyl ester (76) (175 mg, 0.735 mmol) in anhydrous dichloromethane (4 ml). The mixture was stirred at ambient temperature for 7 hours then saturated aqueous sodium hydrogen carbonate solution (40 ml) and dichloromethane (60 ml) were added. The phases were mixed and separated and the organic phase washed with 10% aqueous sodium hydroxide solution (40 ml), dried Na₂SO₄) and evaporated in vacuo to afford a residue (185 mg). The residue was dissolved in ethanol (6.8 ml) and cooled to 0° C. 10% Palladium on carbon (84 mg) was added to the mixture and the atmosphere purged with hydrogen gas. The mixture was stirred overnight under a hydrogen atmosphere at ambient temperature, filtered over celite and the filter cake washed with excess ethyl acetate. The filtrate was concentrated in vacuo, and the residue suspended in a solution of sodium carbonate (193 mg, 1.82 mmol) in water (4 ml). 1,4-Dioxane (2 ml) was added and the mixture cooled to 0° C., then a solution of 9-fluorenylmethyl chloroformate (198 mg, 0.77 mmol) in 1,4-dioxane (2 ml) added in small portions over 40 minutes. The mixture was then allowed to warm to ambient temperature over 40 minutes. Water (40 ml) was added and the product extracted into dichloromethane (3×40 ml). The combined organic layers were dried Na₂SO₄) and evaporated in vacuo to afford a residue (335 mg). Flash chromatography of the residue over silica gel (35 g) eluting with ethyl acetate:heptane mixtures 1:4 followed by 1:1 gave (3S,3aS,6aR)-3-hydroxyhexahydropyrrolo[3,2-b]pyrrole-1,4-dicarboxylic acid 1-tert-butyl ester 4-(9H-fluoren-9-ylmethyl) ester (81) (90 mg, 27%). TLC (Single spot, R_{f}=0.24, EtOAc:heptane 1:1), analytical HPLC Rₜ=16.348 min; HPLC-MS 451.2 [M+H]⁺, 473.2 [M+Na]⁺, 923.4 [2M+Na]⁺; d_{H} (500 MHz, CDCl₃) 1.36-1.49 (9H, s, C(CH₃)₃), 1.65-2.25 (3H, m, BocNCHCH₂ and OH), 2.85-3.68 (5H, m, FmocNCH₂, BocNCH₂ and FmocNCH), 4.05-4.80 (5H, m, OCH₂CH, OCH₂, CHOH and BocNCH), 7.26-7.45 (4H, m, Fmoc aromatic CH), 7.53-7.64 (2H, m, Fmoc aromatic CH) and 7.73-7.80 (2H, m, Fmoc aromatic CH);

### Preparation of (3aS,6aR)-3-oxo-hexahydropyrrolo[3,2-b]pyrrole-1,4-dicarboxylic acid 1-tert-butyl ester 4-(9H-fluoren-9-ylmethyl) ester (6)

(3S,3aS,6aR)-3-Hydroxyhexahydropyrrolo[3,2-b]pyrrole-1,4-dicarboxylic acid 1-tert-butyl ester 4-(9H-fluoren-9-ylmethyl) ester (81) (0.495 g, 1.10 mmol) was dissolved in anhydrous dichloromethane (18 ml) with stirring under argon. Dess-Martin periodinane (0.962 g, 2.27 mmol) was added and the mixture stirred for 4 hours. The mixture was concentrated in vacuo and the residue purified by flash chromatography over silica, eluting with ethyl acetate:heptane mixtures to give (3aS,6aR)-3-oxo-hexahydropyrrolo[3,2-b]pyrrole-1,4-dicarboxylic acid 1-tert-butyl ester 4-(9H-fluoren-9-ylmethyl) ester (6) (0.480 g, 97%) as a white crystalline solid. TLC (R_{f}=0.38, EtOAc:heptane 1:1), analytical HPLC single broad main peak, Rₜ=20.27-21.79 min., HPLC-MS 393.1 [M+2H−Bu)⁺, 449.1 [M+H]⁺, 471.1 [M+Na]⁺, 919.2 [2M+Na]⁺; Elemental analysis C₂₆H₂₈N₂O₅.0.25EtOAc req. (fnd.) % C 68.96 (68.88), % H 6.43 (6.61), % N 5.95 (5.95); HRMS C₂₆H₂₈N₂O₅Na req. 471.1896, fnd. 471.1903 (1.44 ppm); δ_{C} (125 MHz, CDCl₃) 28.36 (C(CH₃)₃), 30.50, 30.93, 31.20 (BocNCHCH₂), 45.68 (FmocNCH₂), 47.20 (FmocCH), 51.71, 52.22 (BocNCH₂), 57.58, 58.64 (BocNCH), 63.03, 63.57 (FmocNCH), 67.70, 68.06 (FmocCH₂), 81.10 (OC(CH₃)₃), 119.94, 124.99, 125.15, 125.29, 127.05, 127.55, 127.71, 127.85 (Fmoc CH aromatics), 143.69, 143.92, 144.23 (Fmoc quaternary aromatics), 153.99, 154.74, 155.04 (2×NCO₂), 206.33, 206.59 (C═O).

Following the general details from Scheme 6, the required bicycle building block (3aS,6aR) 3-Oxo-hexahydro-pyrrolo[3,2-b]pyrrole-1,4-dicarboxylic acid 1-tert-butyl ester 4-(9H-fluoren-9-ylmethyl) ester (6) was converted to building block-linker construct (27) as follows:
A solution of sodium acetate trihydrate (42 mg, 0.311 mmol) in water (0.5 ml) was added to a solution of (3aS,6aR) 3-oxo-hexahydropyrrolo[3,2-b]pyrrole-1,4-dicarboxylic acid 1-tert-butyl ester 4-(9H-fluoren-9-ylmethyl) ester (18.6 mg, 0.0415 mmol) and 4-[[(hydrazinocarbonyl)amino]methyl)cyclohexane carboxylic acid. trifluoroacetate (Murphy, A. M., et al, J. Am. Chem. Soc, 114, 3156-3157, 1992) (68 mg, 0.208 mmol) in ethanol (2.0 ml). Remaining traces of sodium acetate were rinsed into the mixture using a further aliquot of ethanol (1.5 ml) then the reaction heated at 75° C. in a sealed tube for 1 hour. The mixture was stood at ambient temperature for 14 hours then heated at 75° C. for 2.5 hours. The product was extracted into chloroform (60 ml) then washed with hydrochloric acid (0.1M, 2×30 ml), saturated aqueous sodium chloride solution (30 ml) then dried (Na₂SO₄) and the solvent removed in vacuo to leave the product as a pale yellow oil (22.9 mg, 86%). Analytical HPLC has main UV peaks with Rt=19.706 and 21.287 mins and HPLC-MS (main UV peaks each with 646.3 [M+H]⁺).

Following the general details from Scheme 6, the required building block-linker construct (27) was attached to the solid phase providing loaded building block-linker construct (28) as follows:

Building block-linker construct (27) (35.5 μmoles mmoles), 2-(1H-benzotriazole-1-yl)-1,1,3,3-tetramethyluroniumhexafluoro phosphate (HBTU, 13.5 mg, 35.5 μmoles mmoles), 1-hydroxybenzotriazole.hydrate and (HOBT, 5.5 mg, 35.5 μmoles mmoles) were dissolved in dimethylformamide (1.5 mL) and N-methylmorpholine (NMM, 7.8 μL, 71 μmoles mmoles) added. After pre-activation for 5 minutes, free amine gears (10×1.3 μmole) were added and left overnight. The spent coupling solution was then added to free amine gears (6×1.3 μmole) and left overnight. Standard washing and analyses indicated quantitative loading.

Following the general details from Scheme 6, the required loaded building block-linker construct (28) was elaborated on the solid phase as follows:

Loaded construct (28) was elaborated to EXAMPLE 1 (3aR,6aS)-N-[(1S)-1-(4-Benzoyl-6-oxo-hexahydro-pyrrolo[3,2-b]pyrrole-1-carbonyl)-3-methyl-butyl]-4-tert-butylbenzamide by standard Fmoc deprotection and sequential rounds of coupling and washing with the appropriate reagents as follows:—
(i) Fmoc-Leu-OH (2×20 eq, overnight and 4 hr), HBTU, HOBT, NMM activation in DMF
(ii) Standard Fmoc deprotection
(iii) 4-tert-butylbenzoic acid (1×10 eq, overnight), HBTU, HOBT, NMM activation in DMF
(iv) Treatment with 35% TFA in dichloromethane for 30 mins, followed by washing with 2×DMF, 1×2% NMM in DMF, 4×DMF, 4×acetonitrile.
(v) Benzoic anhydride (20 eq) and NMM (5 eq) in DMF for 20 hr.

The crude example was cleaved and analysed (see general techniques). HPLC Rt=18.879-19.62 mins (>90%), HPLC-MS 504.3 [M+H]⁺, 1029.5 [2M+Na]⁺.

The solid phase experimental detailed for EXAMPLE 1 may be followed to couple with a vast range of aminoacids, carboxylic acids, sulphonyl chlorides etc to provide a vast range of analogues of general formula I.

In certain combinations of groupings, the order of solid phase events is amended. For example, when preparing EXAMPLE 194, the U substituent contains an amine group that requires protection via the Boc group, thus the following order of events is utilised:—
(vi) Fmoc-Leu-OH (2×20 eq, overnight and 4 hr), HBTU, HOBT, NMM activation in DMF
(vii) Treatment with 35% TFA in dichloromethane for 30 mins, followed by washing with 2×DMF, 1×2% NMM in DMF, 4×DMF, 4×acetonitrile.
(viii) Benzoic anhydride (20 eq) and NMM (5 eq) in DMF for 20 hr.
(ix) Standard Fmoc deprotection
(x) 4-(4-Carboxyphenyl)-piperazine-1-carboxylic acid tert-butyl ester sodium salt (1×10 eq, overnight), HBTU, HOBT, NMM activation in DMF.
(xi) Standard cleavage

As a further variation, when preparing EXAMPLE 151, the following order of events is utilised
(xii) Fmoc-Leu-OH (2×20 eq, overnight and 4 hr), HBTU, HOBT, NMM activation in DMF
(xiii) Standard Fmoc deprotection
(xiv) 4-Dimethylaminobenzoic acid (1×10 eq, overnight), HBTU, HOBT, NMM activation in DMF
(xv) Washing with 1×DMF, 1×20% piperidine in DMF, 4×DMF, 4×acetonitrile.
(xvi) Treatment with 35% TFA in dichloromethane for 30 mins, followed by washing with 2×DMF, 1×2% NMM in DMF, 4×DMF, 4×acetonitrile.
(xvii) Fmoc-Leu-OH (20 eq, overnight), HBTU, HOBT, NMM activation in DMF.
(xviii) Standard Fmoc deprotection
(xix) Acetic anhydride (50 eq) and NMM (25 eq) in DMF for 1 hr.
(xx) Washing with 1×DMF, 1×20% piperidine in DMF, 4×DMF, 4×acetonitrile.
(xxi) Standard cleavage

As a further variation, when preparing EXAMPLE 80, the following order of events is utilised:—
(xxii) Fmoc-Leu-OH (2×20 eq, overnight and 4 hr), HBTU, HOBT, NMM activation in DMF
(xxiii) Treatment with 35% TFA in dichloromethane for 30 mins, followed by washing with 2×DMF, 1×2% NMM in DMF, 4×DMF, 4×acetonitrile.
(xxiv) Pyridine-2-carboxylic acid (20 eq) and NMM (5 eq) in DMF for 20 hr.
(xxv) Washing with 2×DMF, 1×2% NMM in DMF, 4×DMF, 4×acetonitrile.
(xxvi) Oxidation with m-chloroperbenzoic acid (5 eq) in DCM for 8 hrs.
(xxvii) Washing with 4×DMF, 4×acetonitrile.
(xxviii) Standard Fmoc deprotection
(xxix) 4-Dimethylaminobenzoic acid (1×10 eq, overnight), HBTU, HOBT, NMM activation in DMF.
(xxx) Washing with 1×DMF, 1×20% piperidine in DMF, 4×DMF, 4×acetonitrile.
(xxxi) Standard cleavage

The following examples (2-248) were prepared as detailed for EXAMPLE 1, coupling with the required reagents to provide the full length molecule (see i→v, or vi→xi, or xii→xxi above, or xxii→xxxi above). For step (v), (viii) and (xix) where the anhydride is not readily available, an R²COOH (20 eq)/HBTU (20 eq)/HOBT (20 eq)/NMM (40 eq) mixture in DMF with overnight coupling may be used or an RNHCOCl (20 eq)/NMM (10 eq) mixture in DMF with overnight coupling may be used or an RNCO (20 eq) mixture in DMF with overnight coupling may be used. Following final coupling where the R² or U groups contain a protonatable nitrogen (e.g. pyridyl or 4-dimethylaminobenzoyl), the solid phase intermediate is treated with 20% piperidine in DMF for 10 mins followed by standard washing protocols prior to cleavage.

### EXAMPLE 2

### (3aR,6aS)-N-[(1S)-1-(4-Benzoyl-6-oxo-hexahydro-pyrrolo[3,2-b]pyrrole-1-carbonyl)-3-methyl-butyl]-4-thiophen-2-yl-benzamide

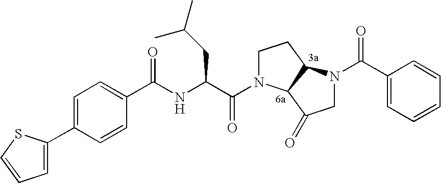

HPLC Rt=18.0-19.2 mins (>90%), HPLC-MS 530.2 [M+H]⁺, 1081.4 [2M+Na]⁺.

### EXAMPLE 3

### (3aR,6aS)-4-tert-Butyl-N-[(1S)-3-methyl-1-(6-oxo-4-phenylacetyl-hexahydro-pyrrolo[3,2-b]pyrrole-1-carbonyl)-butyl]-benzamide

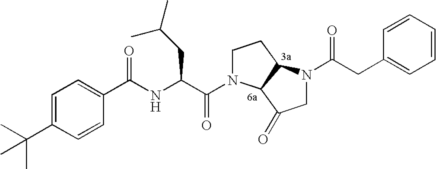

HPLC Rt=19.1-20.2 mins (>90%), HPLC-MS 518.3 [M+H]⁺, 1057.6 [2M+Na]⁺.

### EXAMPLE 4

### (3aR,6aS)-4-tert-Butyl-N-{(1S)-3-methyl-1-[6-oxo-4-(pyridine-2-carbonyl)-hexahydro-pyrrolo[3,2-b]pyrrole-1-carbonyl]-butyl}-benzamide

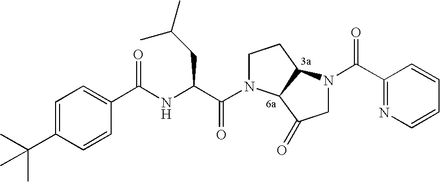

HPLC Rt=17.2-18.1 mins (>90%), HPLC-MS 505.3 [M+H]⁺, 1031.5 [2M+Na]⁺.

### EXAMPLE 5

### (3aR,6aS)-4-tert-Butyl-N-{(1S)-3-methyl-1-[6-oxo-4-(pyridine-3-carbonyl)-hexahydro-pyrrolo[3,2-b]pyrrole-1-carbonyl]-butyl}-benzamide

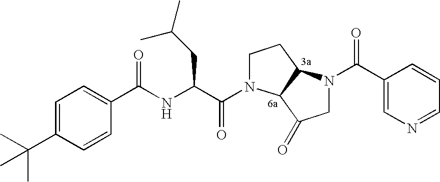

HPLC Rt=15.2-16.4 mins (>90%), HPLC-MS 505.3 [M+H]⁺, 1031.5 [2M+Na]⁺.

### EXAMPLE 6

### (3aR,6aS)-4-tert-Butyl-N-{(1S)-3-methyl-1-[6-oxo-4-(pyridine-4-carbonyl)-hexahydro-pyrrolo[3,2-b]pyrrole-1-carbonyl]-butyl}-benzamide

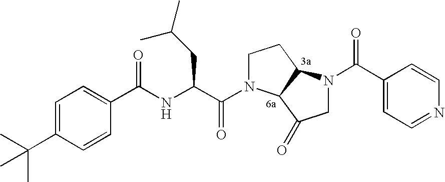

HPLC Rt=15.3-16.4 mins (>90%), HPLC-MS 505.3 [M+H]⁺, 523.3 [M+H+H₂O]⁺.

### EXAMPLE 7

### (3aR,6aS)-Benzo[b]thiophene-2-carboxylic acid [(1S)-1-(4-benzoyl-6-oxo-hexahydro-pyrrolo[3,2-b]pyrrole-1-carbonyl)-3-methyl-butyl]-amide

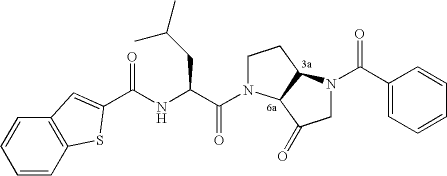

HPLC Rt=16.8-17.9 mins (>90%), HPLC-MS 504.2 [M+H]⁺.

### EXAMPLE 8

### (3aR,6aS)-Quinoline-2-carboxylic acid [(1S)-1-(4-benzoyl-6-oxo-hexahydro-pyrrolo[3,2-b]pyrrole-1-carbonyl)-3-methyl-butyl]-amide

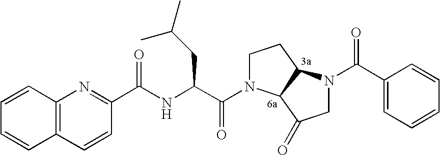

HPLC Rt=17.0-17.8 mins (>90%), HPLC-MS 499.1 [M+H]⁺.

### EXAMPLE 9

### (3aR,6aS)-Benzofuran-2-carboxylic acid [(1S)-1-(4-benzoyl-6-oxo-hexahydropyrrolo[3,2-b]pyrrole-1-carbonyl)-3-methyl-butyl]-amide

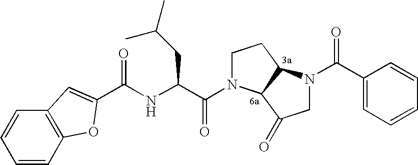

HPLC Rt=16.2-17.7 mins (>90%), HPLC-MS 488.1 [M+H]⁺, 997.2 [2M+Na]⁺.

### EXAMPLE 10

### (3aR,6aS)-3-Methyl-benzofuran-2-carboxylic acid [(1S)-1-(4-benzoyl-6-oxo-hexahydro-pyrrolo[3,2-b]pyrrole-1-carbonyl)-3-methyl-butyl]-amide

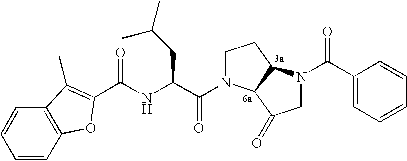

HPLC Rt=17.8-18,9 mins (>85%), HPLC-MS 502.1 [M+H]⁺, 520.1 [M+H+H₂O]⁺.

### EXAMPLE 11

### (3aR,6aS)-N-[(1S)-1-(4-Benzoyl-6-oxo-hexahydro-pyrrolo[3,2-b]pyrrole-1-carbonyl)-butyl]-4-tert-butyl-benzamide

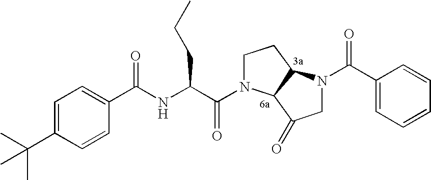

HPLC Rt=17.4-18.2 mins (>90%), HPLC-MS 490.2 [M+H]⁺.

### EXAMPLE 12

### (3aR,6aS)-Quinoxaline-2-carboxylic acid [(1S)-1-(4-benzoyl-6-oxo-hexahydro-pyrrolo[3,2-b]pyrrole-1-carbonyl)-3-methyl-butyl]-amide

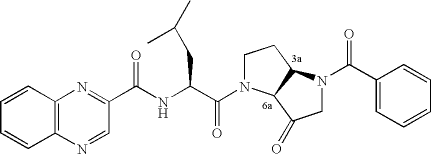

HPLC Rt=15.6-16.7 mins (>90%), HPLC-MS 500.2 [M+H]⁺.

### EXAMPLE 13

### (3aR,6aS)-N-[(1S)-1-(4-Benzoyl-6-oxo-hexahydro-pyrrolo[3,2-b]pyrrole-1-carbonyl)-3-methyl-butyl]-3-phenoxy-benzamide

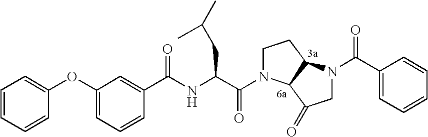

HPLC Rt=18.5-19.7 mins (>80%), HPLC-MS 540.1 [M+H]⁺.

### EXAMPLE 14

### (3aR,6aS)-N-[(1S)-1-(4-Benzoyl-6-oxo-hexahydro-pyrrolo[3,2-b]pyrrole-1-carbonyl)-3-methyl-butyl]-4-dimethylamino-benzamide

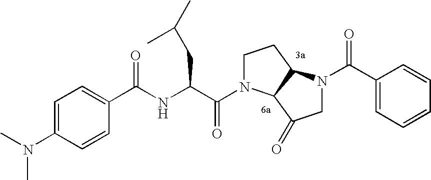

HPLC Rt=12.16 mins (>90%), HPLC-MS 491.2 [M+H]⁺, 509.2 [M+H+H₂O]⁺.

### EXAMPLE 15

### (3aR,6aS)-Benzo[b]thiophene-2-carboxylic acid {(1S)-3-methyl-1-[6-oxo-4-(pyridine-3-carbonyl)-hexahydro-pyrrolo[3,2-b]pyrrole-1-carbonyl]-butyl}-amide

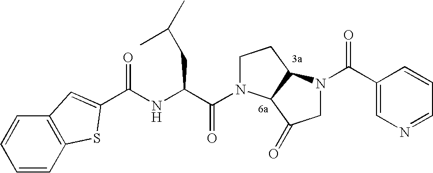

HPLC Rt=13.93 mins (>95%), HPLC-MS 505.2 [M+H]⁺.

### EXAMPLE 16

### (3aR,6aS)-Benzofuran-2-carboxylic acid {(1S)-3-methyl-1-[6-oxo-4-(pyridine-3-carbonyl)-hexahydro-pyrrolo[3,2-b]pyrrole-1-carbonyl]-butyl}-amide

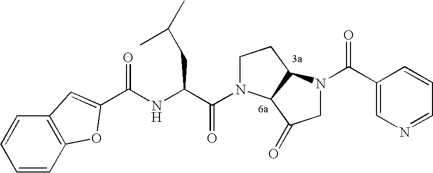

HPLC Rt=13.42 mins (>85%), HPLC-MS 489.2 [M+H]⁺, 999.4 [2M+Na]⁺.

### EXAMPLE 17

### (3aR,6aS)-3-Methyl-benzofuran-2-carboxylic acid {(1S)-3-methyl-1-[6-oxo-4-(pyridine-3-carbonyl)-hexahydro-pyrrolo[3,2-b]pyrrole-1-carbonyl]-butyl}-amide

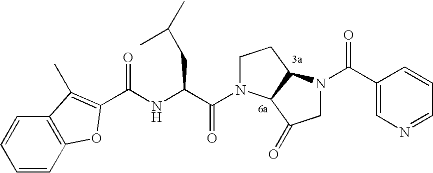

HPLC Rt=14.58 mins (>90%), HPLC-MS 503.2 [M+H]⁺.

### EXAMPLE 18

### (3aR,6aS)-Quinoline-2-carboxylic acid {(1S)-3-methyl-1-[6-oxo-4-(pyridine-3-carbonyl)-hexahydro-pyrrolo[3,2-b]pyrrole-1-carbonyl]-butyl}-amide

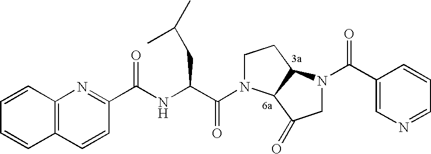

HPLC Rt=14.00 mins (>90%), HPLC-MS 500.2 [M+H]⁺.

### EXAMPLE 19

### (3aR,6aS)-Quinoxaline-2-carboxylic acid {(1S)-3-methyl-1-[6-oxo-4-(pyridine-3-carbonyl)-hexahydro-pyrrolo[3,2-b]pyrrole-1-carbonyl]-butyl}-amide

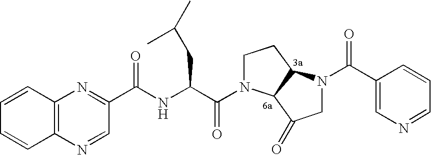

HPLC Rt=12.77 mins (>90%), HPLC-MS 501.2 [M+H]⁺.

### EXAMPLE 20

### (3aR,6aS)-N-{(1S)-3-Methyl-1-[6-oxo-4-(pyridine-3-carbonyl)-hexahydro-pyrrolo[3,2-b]pyrrole-1-carbonyl]-butyl}-3-phenoxy-benzamide

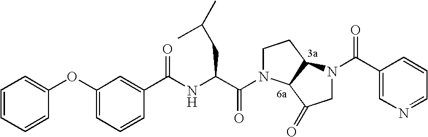

HPLC Rt=16.06 mins (>85%), HPLC-MS 541.2 [M+H]⁺.

### EXAMPLE 21

### (3aR,6aS)-4-Dimethylamino-N-{(1S)-3-methyl-1-[6-oxo-4-(pyridine-3-carbonyl)-hexahydro-pyrrolo[3,2-b]pyrrole-1-carbonyl]-butyl}-benzamide

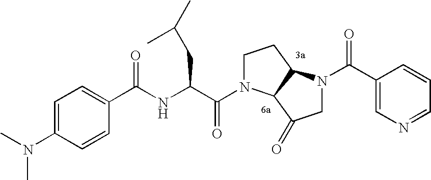

HPLC Rt=8.70 mins (>95%), HPLC-MS 255.6 [M+2H+H₂O]²⁺, 492.2 [M+H]⁺, 510.2 [M+H+H₂O]⁺.

### EXAMPLE 22

### (3aR,6aS)-N-[(1S)-2-(4-Benzoyl-6-oxo-hexahydro-pyrrolo[3,2-b ] pyrrol-1-yl)-2-oxo-1-thiophen-2-ylmethyl-ethyl]-4-tert-butyl-benzamide

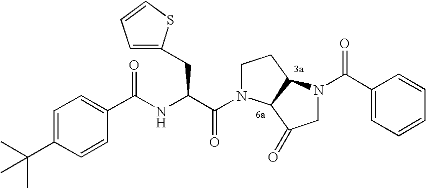

HPLC Rt=18.0-19.0 mins (>80%), HPLC-MS 544.2 [M+H]⁺.

### EXAMPLE 23

### (3aR,6aS)-4-tert-Butyl-N-{(1S)-2-oxo-2-[6-oxo-4-(pyridine-3-carbonyl)-hexahydro-pyrrolo[3,2-b]pyrrole-1-yl]-1-thiophen-2-ylmethyl-ethyl}-benzamide

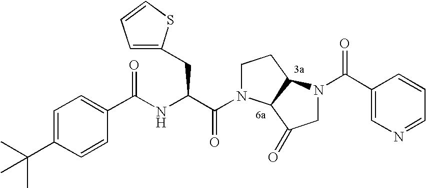

HPLC Rt=15.626 mins (>85%), HPLC-MS 545.2 [M+H]⁺.

### EXAMPLE 24

### (3aR,6aS)-4-tert-Butyl-N-{(1S)-3-methyl-1-[6-oxo-4-(3-phenoxy-benzoyl)-hexahydro-pyrrolo[3,2-b]pyrrole-1-carbonyl]-butyl}-benzamide

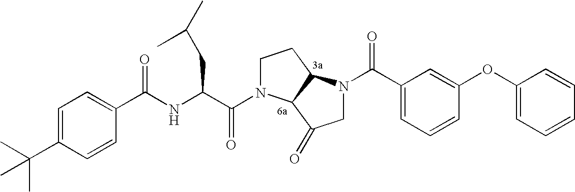

HPLC Rt=22.0-23.2 mins (>75%), HPLC-MS 596.1 [M+H]⁺.

### EXAMPLE 25

### (3aR,6aS)-N-{(1S)-1-[4-(3-Bromo-benzoyl)-6-oxo-hexahydro-pyrrolo[3,2-b]pyrrole-1-carbonyl]-3-methyl-butyl}-4-tert-butyl-benzamide

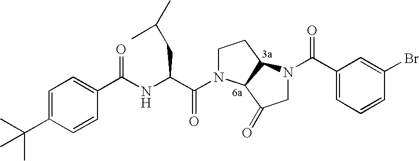

HPLC Rt=20.3-21.5 mins (>80%), HPLC-MS 582.1/584.1 [M+H]⁺.

### EXAMPLE 26

### (3aR,6aS)-4-tert-Butyl-N-{(1S)-3-methyl-1-[6-oxo-4-(4-[1,2,4] triazol-1-yl-benzoyl)-hexahydro-pyrrolo[3,2-b]pyrrole-1-carbonyl]-butyl}-benzamide

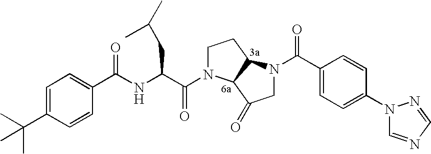

HPLC Rt=17.4-8.7 mins (>80%), HPLC-MS 571.1 [M+H]⁺.

### EXAMPLE 27

### (3aR,6aS)-4-tert-Butyl-N-{(1S)-3-methyl-1-[6-oxo-4-(2-phenyl-thiazole-4-carbonyl)-hexahydro-pyrrolo[3,2-b]pyrrole-1-carbonyl]-butyl}-benzamide

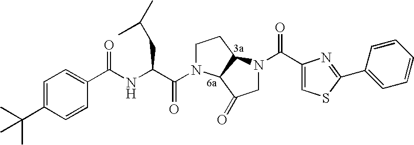

HPLC Rt=21.4-22.7 mins (>80%), HPLC-MS 587.1 [M+H]⁺.

### EXAMPLE 28

### (3aR,6aS)-4-tert-Butyl-N-{(1S)-3-methyl-1-[6-oxo-4-(5-phenyl-thiophene-2-carbonyl)-hexahydro-pyrrolo[3,2-b]pyrrole-1-carbonyl]-butyl}-benzamide

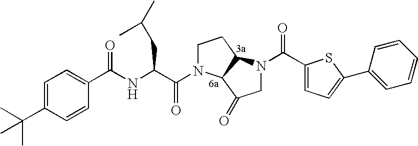

HPLC Rt=22.0-23.0 mins (>70%), HPLC-MS 586.1 [M+H]⁺.

### EXAMPLE 29

### (3aR,6aS)-4-tert-Butyl-N-{(1S)-1-[4-(2,3-dihydro benzo[1,4]dioxine-2-carbonyl)-6-oxo-hexahydro-pyrrolo[3,2-b]pyrrole-1-carbonyl]-3-methyl-butyl}-benzamide

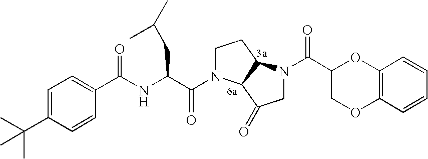

HPLC Rt=20.1-21.3 mins (>80%), HPLC-MS 562.1 [M+H]⁺.

### EXAMPLE 30

### (3aR,6aS)-4-tert-Butyl-N-{(1S)-1-[4-(2,3-dihydro-benzo[1,4]dioxine-6-carbonyl)-6-oxo-hexahydro-pyrrolo[3,2-b]pyrrole-1-carbonyl]-3-methyl-butyl}-benzamide

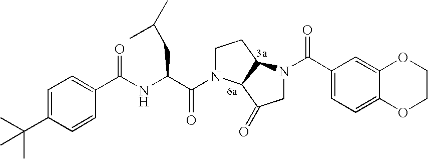

HPLC Rt=18.3-19.1 mins (>85%), HPLC-MS 562.1 [M+H]⁺.

### EXAMPLE 31

### (3aR,6aS)-N-{(1S)-1-[4-(Benzo[b]thiophene-3-carbonyl)-6-oxo-hexahydro-pyrrolo[3,2-b]pyrrole-carbonyl]-3-methyl-butyl}-4-tert-butyl-benzamide

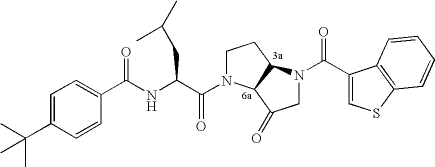

HPLC Rt=20.5-21.8 mins (>80%), HPLC-MS 560.2 [M+H]⁺.

### EXAMPLE 32

### (3aR,6aS)-N-{(1S)-1-[4-(Benzothiazole-6-carbonyl)-6-oxo-hexahydro-pyrrolo[3,2-b]pyrrole-1-carbonyl]-3-methyl-butyl}-4-tert-butyl-benzamide

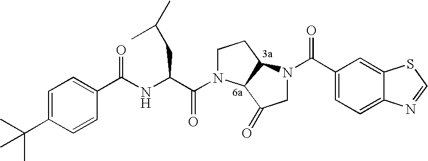

HPLC Rt=17.9-18.9 mins (>75%), HPLC-MS 561.2 [M+H]⁺.

### EXAMPLE 33

### (3aR,6aS)-tert-Butyl-N-{(1)-3-methyl-1-[4-(naphthalene-1-carbonyl)-6-oxo-hexahydro-pyrrolo[3,2-b]pyrrole-1-carbonyl-]-butyl}-benzamide

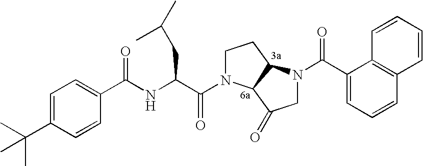

HPLC Rt=20.5-21.7 mins (>80%), HPLC-MS 554.1 [M+H]⁺.

### EXAMPLE 34

### (3aR,6aS)-4-tert-Butyl-N-{(1S)-3-methyl-1-[4-(naphthalene-2-carbonyl)-6-oxo-hexahydro-pyrrolo[3,2-b]pyrrole-1-carbonyl]-butyl}-benzamide

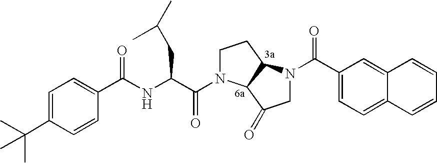

HPLC Rt=20.7-21.8 mins (>75%), HPLC-MS 554.2 [M+H]⁺.

### EXAMPLE 35

### (3aR,6aS)-4-tert-Butyl-N-{(1S)-3-methyl-1-[6-oxo-4-(5-thiophen-2-yl-pyridine-3-carbonyl)-hexahydro-pyrrolo[3,2-b]pyrrole-1-carbonyl]-butyl}-benzamide

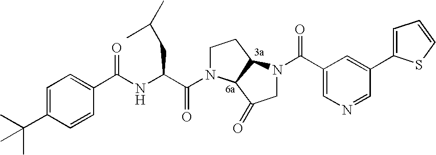

HPLC Rt=18.9-19.8 mins (>70%), HPLC-MS 587.1 [M+H]⁺.

### EXAMPLE 36

### (3aR,6aS)-N-{(1S)-1-[4-(2-Benzyl-benzoyl)-6-oxo-hexahydro-pyrrolo[3,2-b]pyrrole-1-carbonyl]-3-methyl-butyl}-4-tert-butyl-benzamide

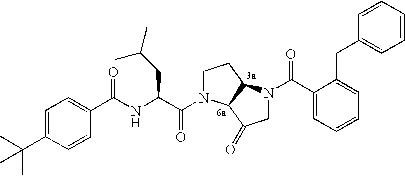

HPLC Rt=21.8-22.9 mins (>80%), HPLC-MS 594.1 [M+H]⁺.

### EXAMPLE 37

### (3aS,6aR)-((1S)-1-{4-[(2S)-2-(4-Dimethylamino-benzoylamino)-4-methyl-pentanoyl]-3-oxo-hexahydro-pyrrolo[3,2-b]pyrrole-1-carbonyl}-3-methyl-butyl)-carbamic acid benzyl ester

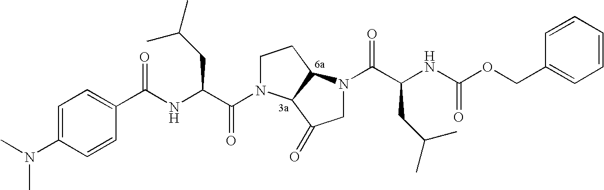

HPLC Rt=17.15 mins (>85%), HPLC-MS 634.3 M+H]⁺.

### EXAMPLE 38

### (3aS,6aR)-(2-{4-[(2S)-2-(4-Dimethylamino-benzoylamino)-4-methyl-pentanoyl]-3-oxo-hexahydro-pyrrolo[3,2-b]pyrrole-1-yl}-2-oxo-ethyl)-carbamic acid benzyl ester

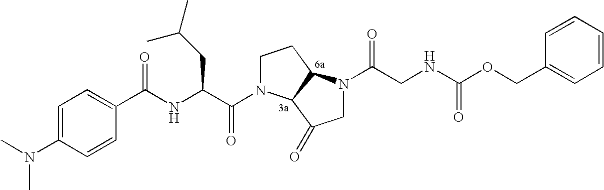

HPLC Rt=13.79 mins (>85%), HPLC-MS 578.3 [M+H]⁺.

### EXAMPLE 39

### (3aR,6aS)-N-[(1S)-1-(4-Benzoyl-6-oxo-hexahydro-pyrrolo[3,2-b]pyrrole-1-carbonyl)-3-methyl-butyl]-4-phenoxy-benzamide

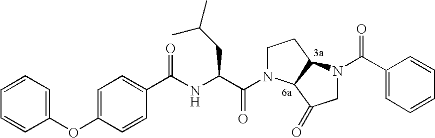

HPLC Rt=17.5-18.2 mins (>75%), HPLC-MS 540.2 [M+H]⁺.

### EXAMPLE 40

### (3aR,6aS)-N-{(1S)-1-[4-(2-Methanesulfonyl-benzoyl)-6-oxo-hexahydro-pyrrolo[3,2-b]pyrrole-1-carbonyl]-3-methyl-butyl}-4-phenoxy-benzamide

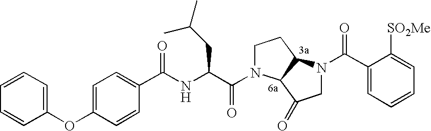

HPLC Rt=16.9-17.9 mins (>75%), HPLC-MS 618.2 [M+H]⁺, 636.2 [M+H+H₂O]⁺.

### EXAMPLE 41

### (3aR,6aS)-N-{(1S)-1-[4-(3-Methanesulfonyl-benzoyl)-6-oxo-hexahydro-pyrrolo[3,2-b]pyrrole-1-carbonyl]-3-methyl-butyl}-4-phenoxy-benzamide

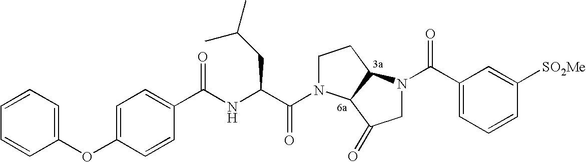

HPLC Rt=16.6-17.7 mins (>70%), HPLC-MS 618.2 [M+H]⁺.

### EXAMPLE 42

### (3aR,6aS)-4-Dimethylamino-N-{(1S)-1-[4-(2-methanesulfonyl-benzoyl)-6-oxo-hexahydro-pyrrolo[3,2-b]pyrrole-1-carbonyl]-3-methyl-butyl}-benzamide

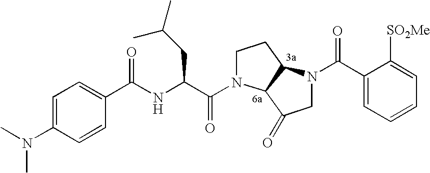

HPLC Rt=13.10 mins (>90%), HPLC-MS 569.3 [M+H]⁺, 1159.4 [2M+Na]⁺.

### EXAMPLE 43

### (3aR,6aS)-4-Dimethylamino-N-{(1S)-1-[4-(3-methanesulfonyl-benzoyl)-6-oxo-hexahydro-pyrrolo[3,2-b]pyrrole-1-carbonyl]-3-methyl-butyl}-benzamide

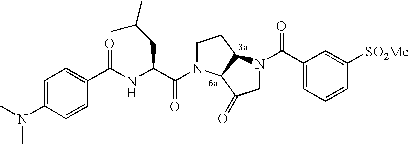

HPLC Rt=11.59 mins (>95%), HPLC-MS 569.2 [M+H]⁺, 587.2 [M+H+H₂O]⁺.

### EXAMPLE 44

### (3aR,6aS)-5-Phenyl-thiophene-2-carboxylic acid [(1S)-1-(4-benzoyl-6-oxo-hexahydro-pyrrolo[3,2-b]pyrrole-1-carbonyl)-3-methyl-butyl]-amide

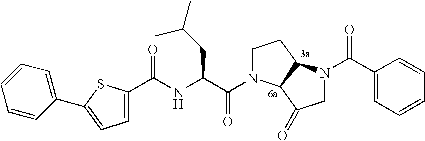

HPLC Rt=17.2-18.1 mins (>75%), HPLC-MS 530.2 [M+H]⁺.

### EXAMPLE 45

### (3aR,6aS)-4-Dimethylamino-N-{(1S)-3-methyl-1-[4-(2-nitro-benzoyl)-6-oxo-hexahydro-pyrrolo[3,2-b]pyrrole-1-carbonyl]-butyl}-benzamide

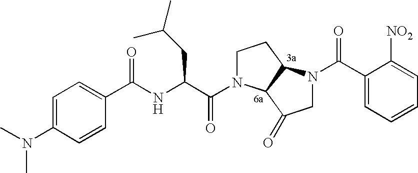

HPLC Rt=12.47 mins (>95%), HPLC-MS 536.2 [M+H]⁺, 554.2 [M+H+H₂O]⁺.

### EXAMPLE 46

### (3aR,6aS)-4-Dimethylamino-N-{(1S)-3-methyl-1-[4-(3-nitro-benzoyl)-6-oxo-hexahydro-pyrrolo[3,2-b]pyrrole-1-carbonyl]-butyl}-benzamide

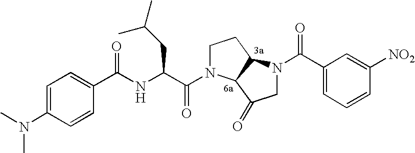

HPLC Rt=13.17 mins (>90%), HPLC-MS 536.2 [M+H]⁺, 554.2 [M+H+H₂O]⁺.

### EXAMPLE 47

### (3aR,6aS)-4-Dimethylamino-N-{(1S)-3-methyl-1-[4-(4-nitro-benzoyl)-6-oxo-hexahydro-pyrrolo[3,2-b]pyrrole-1-carbonyl]-butyl}-benzamide

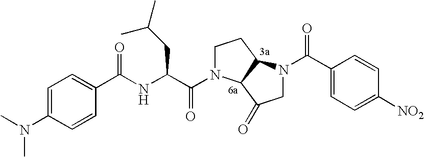

HPLC Rt=13.19 mins (>95%), HPLC-MS 536.2 [M+H]⁺, 554.2 [M+H+H₂O]⁺.

### EXAMPLE 48

### (3aR,6aS)-N-{(1S)-1-[4-(Adamantane-1-carbonyl)-6-oxo-hexahydro-pyrrolo[3,2-b]pyrrole-1-carbonyl]-3-methyl-butyl}-4-dimethylamino-benzamide

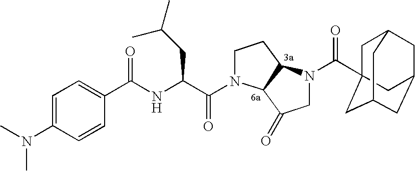

HPLC Rt=16.4-17.1 mins (>50%), HPLC-MS 549.3 [M+H]⁺.

### EXAMPLE 49

### (3aR,6aS)-4-Dimethylamino-N-{(1S)-1-[4-(4-hydroxy-benzoyl)-6-oxo-hexahydro-pyrrolo[3,2-b]pyrrole-1-carbonyl]-3-methyl-butyl}-benzamide

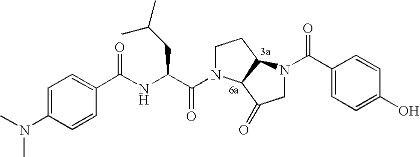

HPLC Rt=11.05 mins (>90%), HPLC-MS 507.2 [M+H]⁺, 525.2 [M+H+H₂O]⁺.

### EXAMPLE 50

### (3aR,6aS)-N-{(1S)-1-[4-(4-Amino-benzoyl)-6-oxo-hexahydro-pyrrolo[3,2-b]pyrrole-1-carbonyl]-3-methyl-butyl}-4-dimethylamino-benzamide

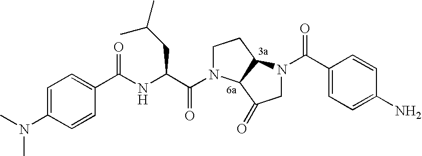

HPLC Rt=9.33 mins (>85%), HPLC-MS 506.2 [M+H]⁺, 524.2 [M+H+H₂O]⁺.

### EXAMPLE 51

### (3aR,6aS)-4-Dimethylamino-N-{(1S)-1-[4-(5-methanesulfonyl-thiophene-2-carbonyl)-6-oxo-hexahydro-pyrrolo[3,2-b]pyrrole-1-carbonyl]-3-methyl-butyl}-benzamide

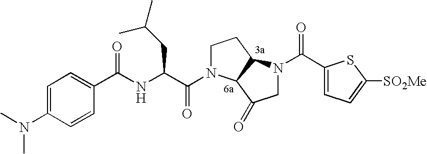

HPLC Rt=12.10 mins (>95%), HPLC-MS 575.1 [M+H]⁺, 593.1 [M+H+H₂O]⁺.

### EXAMPLE 52

### (3aR,6aS)-4-Dimethylamino-N-{(1S)-3-methyl-1-[6-oxo-4-(quinoline-6-carbonyl)-hexahydro-pyrrolo[3,2-b]pyrrole-1-carbonyl]-butyl}-benzamide

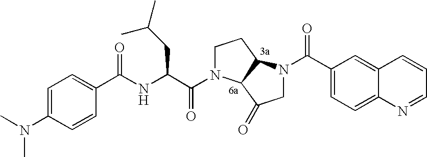

HPLC Rt=9.99 mins (>85%), HPLC-MS 542.2 [M+H]⁺, 560.2 [M+H+H₂O]⁺.

### EXAMPLE 53

### (3aR,6aS)-4-Dimethylamino-N-{(1S)-3-methyl-1-[4-(2-methyl-benzoyl)-6-oxo-hexahydro-pyrrolo[3,2-b]pyrrole-1-carbonyl]-butyl}-benzamide

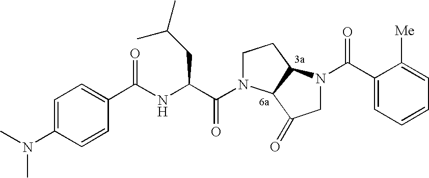

HPLC Rt=13.08 mins (>90%), HPLC-MS 505.2 [M+H]⁺, 523.2 [M+H+H₂O]⁺.

### EXAMPLE 54

### (3aR,6aS)-4-Dimethylamino-N-{(1S)-3-methyl-1-[4-(3-methyl-benzoyl)-6-oxo-hexahydro-pyrrolo[3,2-b]pyrrole-1-carbonyl]-butyl}-benzamide

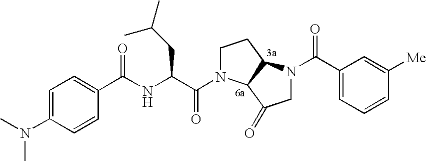

HPLC Rt=13.12 mins (>85%), HPLC-MS 505.2 [M+H]⁺, 523.2 [M+H+H₂O]⁺.

### EXAMPLE 55

### (3aR,6aS)-4-Dimethylamino-N-{(1S)-3-methyl-1-[4-(4-methyl-benzoyl)-6-oxo-hexahydro-pyrrolo[3,2-b]pyrrole-1-carbonyl]-butyl}-benzamide

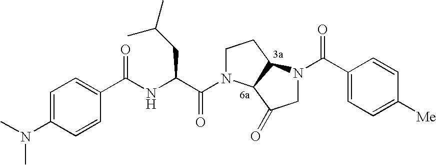

HPLC Rt=13.65 mins (>90%), HPLC-MS 505.2 [M+H]⁺, 523.2 [M+H+H₂O]⁺.

### EXAMPLE 56

### (3aR,6aS)-4-Dimethylamino-N-{(1S)-3-methyl-1-[6-oxo-4-(4-phenoxy-benzoyl)-hexahydro-pyrrolo[3,2-b]pyrrole-1-carbonyl]-butyl}-benzamide

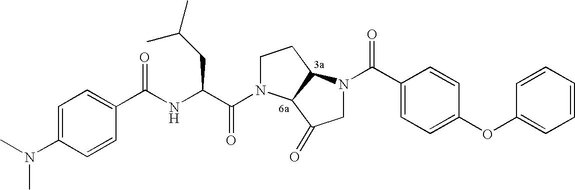

HPLC Rt=16.38 mins (>80%), HPLC-MS 583.2 [M+H]⁺.

### EXAMPLE 57

### (3aR,6aS)-4-Dimethylamino-N-{(1S)-1-[4-(4-fluoro-benzoyl)-6-oxo-hexahydro-pyrrolo[3,2-b]pyrrole-1-carbonyl]-3-methyl-butyl}-benzamide

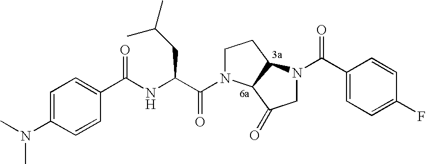

HPLC Rt=12.83 mins (>90%), HPLC-MS 509.1 [M+H]⁺, 527.1 [M+H+H₂O]⁺.

### EXAMPLE 58

### (3aR,6aS)-N-{(1S)-1-[4-(3-Methanesulfonyl-benzoyl)-6-oxo-hexahydro-pyrrolo[3,2-b]pyrrole-1-carbonyl]-3-methyl-butyl}-4-nitro-benzamide

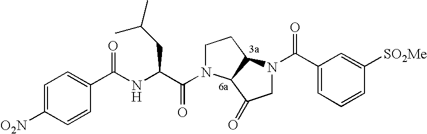

HPLC Rt=13.37 mins (>95%), HPLC-MS 571.1 [M+H]⁺.

### EXAMPLE 59

### (3aR,6aS)-3-Dimethylamino-N-{(1S)-1-[4-(3-methanesulfonyl-benzoyl)-6-oxo-hexahydro-pyrrolo[3,2-b]pyrrole-1-carbonyl]-3-methyl-butyl}-benzamide

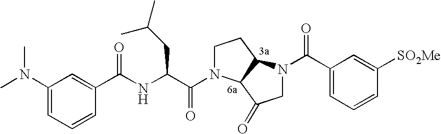

HPLC Rt=10.29 mins (>85%), HPLC-MS 569.1 [M+H]⁺, 587.2 [M+H+H₂O]⁺.

### EXAMPLE 60

### (3aR,6aS)-4-Diethylamino-N-{(1S)-1-[4-(3-methanesulfonyl-benzoyl)-6-oxo-hexahydro-pyrrolo[3,2-b]pyrrole-1-carbonyl]-3-methyl-butyl}-benzamide

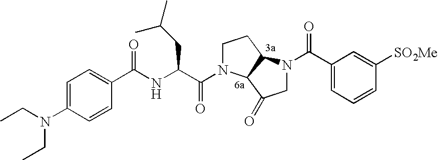

HPLC Rt=11.13 mins (>90%), HPLC-MS 597.2 [M+H]⁺, 615.2 [M+H+H₂O]⁺.

### EXAMPLE 61

### (3aR,6aS)-4-Amino-N-{(1S)-1-[4-(3-methanesulfonyl-benzoyl)-6-oxo-hexahydro-pyrrolo[3,2-b]pyrrole-1-carbonyl]-3-methyl-butyl}-benzamide

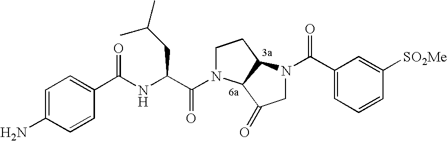

HPLC Rt=9.69 mins (>80%), HPLC-MS 541.1 [M+H]⁺.

### EXAMPLE 62

### (3aR,6aS)-4-Dimethylamino-N-{(1S)-1-[4-(2-fluoro-benzoyl)-6-oxo-hexahydro-pyrrolo[3,2-b]pyrrole-1-carbonyl]-3-methyl-butyl}-benzamide

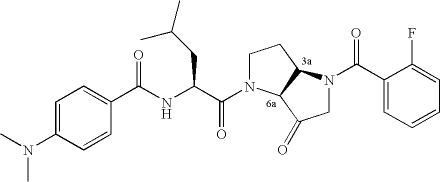

HPLC Rt=9.18 mins (>95%), HPLC-MS 509.2 [M+H]⁺, 527.2 [M+H+H₂O]⁺.

### EXAMPLE 63

### (3aR,6aS)-4-Dimethylamino-N-{(1S)-1-[4-(3-fluoro-benzoyl)-6-oxo-hexahydro-pyrrolo[3,2-b]pyrrole-1-carbonyl]-3-methyl-butyl}-benzamide

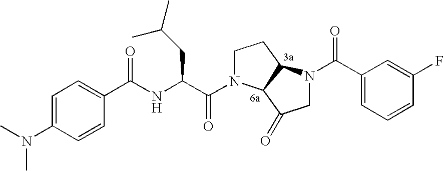

HPLC Rt=10.59 mins (>90%), HPLC-MS 509.2 [M+H]⁺, 527.2 [M+H+H₂O]⁺.

### EXAMPLE 64

### (3aR,6aS)-4-Dimethylamino-N-{(1S)-1-[4-(2-methoxy-benzoyl)-6-oxo-hexahydro-pyrrolo[3,2-b]pyrrole-1-carbonyl]-3-methyl-butyl}-benzamide

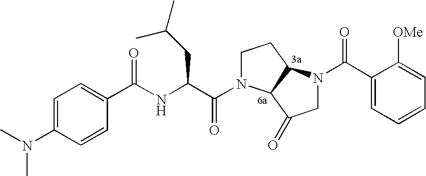

HPLC Rt=11.20 mins (>95%), HPLC-MS 521.2 [M+H]⁺.

### EXAMPLE 65

### (3aR,6aS)-4-Dimethylamino-N-{(1S)-1-[4-(3-methoxy-benzoyl)-6-oxo-hexahydro-pyrrolo[3,2-b]pyrrole-1-carbonyl]-3-methyl-butyl}-benzamide

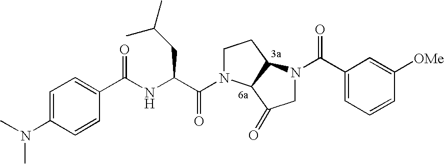

HPLC Rt=12.5 mins (>90%), HPLC-MS 521.2 [M+H]⁺.

### EXAMPLE 66

### (3aR,6aS)-4-Dimethylamino-N-{(1S)-1-[4-(4-methoxy-benzoyl)-6-oxo-hexahydro-pyrrolo[3,2-b]pyrrole-1-carbonyl]-3-methyl-butyl}-benzamide

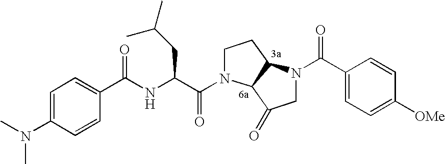

HPLC Rt=13.33 mins (>95%), HPLC-MS 521.1 [M+H]⁺.

### EXAMPLE 67

### (3aR,6aS)-4-Dimethylamino-N-{(1S)-3-methyl-1-[6-oxo-4-(2-trifluoromethyl-benzoyl)-hexahydro-pyrrolo[3,2-b]pyrrole-1-carbonyl]-butyl}-benzamide

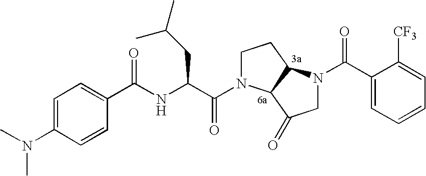

HPLC Rt=10.98 mins (>95%), HPLC-MS 559.2 [M+H]⁺, 577.2 [M+H+H₂O]⁺.

### EXAMPLE 68

### (3aR,6aS)-4-Dimethylamino-N-{(1S)-3-methyl-1-[6-oxo-4-(3-trifluoromethyl-benzoyl)-hexahydro-pyrrolo[3,2-b]pyrrole-1-carbonyl]-butyl}-benzamide

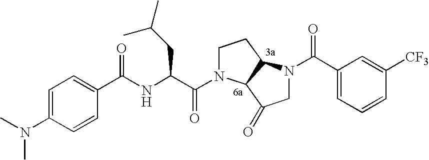

HPLC Rt=12.11 mins (>95%), HPLC-MS 559.2 [M+H]⁺, 577.2 [M+H+H₂O]⁺.

### EXAMPLE 69

### (3aR,6aS)-4-Dimethylamino-N-{(1S)-3-methyl-1-[6-oxo-4-(4 trifluoromethyl-benzoyl)-hexahydro-pyrrolo[3,2-b]pyrrole-1-carbonyl]-butyl}-benzamide

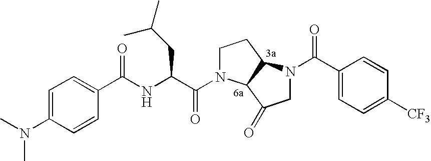

HPLC Rt=9.76 mins (>95%), HPLC-MS 559.2 [M+H]⁺, 577.2 [M+H+H₂O]⁺.

### EXAMPLE 70

### (3aR,6aS)-4-Dimethylamino-N-{(1S)-3-methyl-1-[6-oxo-4-(3-trifluoromethoxy-benzoyl)-hexahydro-pyrrolo[3,2-b]pyrrole-1-carbonyl]-butyl}-benzamide

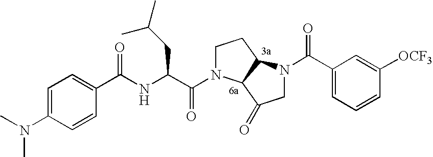

HPLC Rt=10.42 mins (>95%), HPLC-MS 575.2 [M+H]⁺, 593.2 [M+H+H₂O]⁺.

### EXAMPLE 71

### (3aR,6aS)-4-Dimethylamino-N-{(1S)-3-methyl-1-[6-oxo-(4-trifluoromethoxy-benzoyl)-hexahydro-pyrrolo[3,2-b]pyrrole-1-carbonyl]-butyl}-benzamide

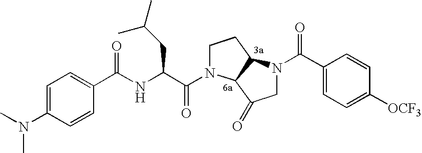

HPLC Rt=10.48 mins (>95%), HPLC-MS 575.2 [M+H]⁺, 593.2 [M+H+H₂O]⁺.

### EXAMPLE 72

### (3aR,6aS)-N-{(1S)-1-[4-(2-Chloro-benzoyl)-6-oxo-hexahydro-pyrrolo[3,2-b]pyrrole-1-carbonyl)-3-methyl-butyl}-4-dimethylamino-benzamide

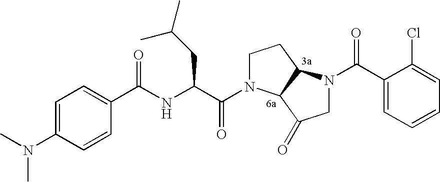

HPLC Rt=9.99 mins (>95%), HPLC-MS 525.2/527.2 [M+H]⁺, 543.2/545.2 [M+H+H₂O]⁺.

### EXAMPLE 73

### (3aR,6aS)-N-{(1S)-1-[4-(3-Chloro-benzoyl)-6-oxo-hexahydro-pyrrolo[3,2-b]pyrrole-1-carbonyl]-3-methyl-butyl}-4-dimethylamino-benzamide

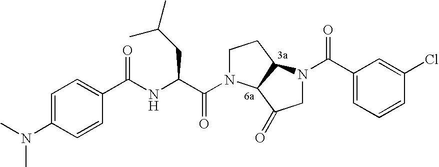

HPLC Rt=11.08 mins (>95%), HPLC-MS 525.2/527.2 [M+H]⁺, 543.2/545.2 [M+H+H₂O]⁺.

### EXAMPLE 74

### (3aR,6aS)-N-{(1S)-1-[4-(4-Chloro-benzoyl)-6-oxo-hexahydro-pyrrolo[3,2-b]pyrrole-1-carbonyl]-3-methyl-butyl}-4-dimethylamino-benzamide

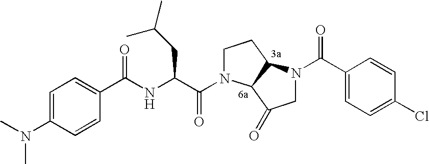

HPLC Rt=11.03 mins (>95%), HPLC-MS 525.2/527.2 [M+H]⁺, 543.2/545.2 [M+H+H₂O]⁺.

### EXAMPLE 75

### (3aR,6aS)-N-{(1S)-1-[4-(4-tert-Butyl-benzoyl)-6-oxo-hexahydro-pyrrolo[3,2-b]pyrrole-1-carbonyl]-3-methyl-butyl}-4-dimethylamino-benzamide

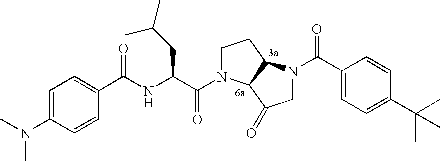

HPLC Rt=11.51 mins (>95%), HPLC-MS 547.3 [M+H]⁺.

### EXAMPLE 76

### (3aS,6aR)-4-{4-[(2S)-2-(4-Dimethylamino-benzoylamino)-4-methyl-pentanoyl]-3-oxo-hexahydro-pyrrolo[3,2-b]pyrrole-1-carbonyl}-benzoic acid methyl ester

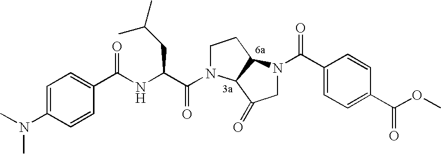

HPLC Rt=9.76 mins (>95%), HPLC-MS 549.2 [M+H]⁺, 567.2 [M+H+H₂O]⁺.

### EXAMPLE 77

### (3aR,6aS)-N-{(1S)-1-[4-(Benzo[1,3]dioxole-5-carbonyl)-6-oxo-hexahydro-pyrrolo[3,2-b]pyrrole-1-carbonyl]-3-methyl-butyl}-4-dimethylamino-benzamide

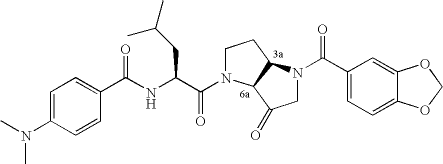

HPLC Rt=12.3 mins (>90%), HPLC-MS 535.2 [M+H]⁺.

### EXAMPLE 78

### (3aR,6aS)-4-Dimethylamino-N-[(1S)-1-(4-diphenylacetyl-6-oxo-hexahydro-pyrrolo[3,2-b]pyrrole-1-carbonyl)-3-methyl-butyl]-benzamide

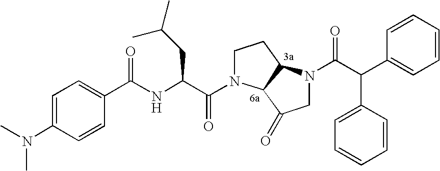

HPLC Rt=14.0-15.1 mins (>85%), HPLC-MS 581.2 [M+H]⁺.

### EXAMPLE 79

### (3aR,6aS)-4-Dimethylamino-N-{(1S)-3-methyl-1-[6-oxo-4-(pyridine-2-carbonyl)-hexahydro-pyrrolo[3,2-b]pyrrole-1-carbonyl]-butyl}-benzamide

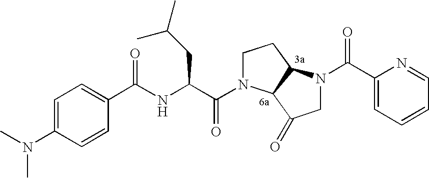

HPLC Rt=6.17 mins (>95%), HPLC-MS 492.2 [M+H]⁺.

### EXAMPLE 80

### (3aR,6aS)-4-Dimethylamino-N-{1S)-3-methyl-1-[6-oxo-4-(1-oxy-pyridine-2-carbonyl)-hexahydro-pyrrolo[3,2-b]pyrrole-1-carbonyl]-butyl}-benzamide

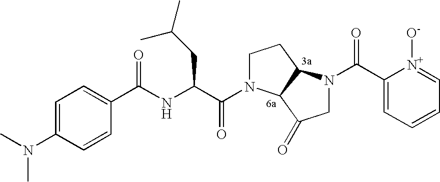

HPLC Rt=5.82 mins (>75%), HPLC-MS 508.2 [M+H]⁺.

Oxidation of the intermediate was performed as detailed in the general soild phase methods prior to compound release from the solid phase.

### EXAMPLE 81

### (3aR,6aS)-4-Dimethylamino-N-{(1S)-3-methyl-1-[6-oxo-4-(1-oxy-pyridine-3-carbonyl)-hexahydro-pyrrolo[3,2-b]pyrrole-1-carbonyl]-butyl}-benzamide

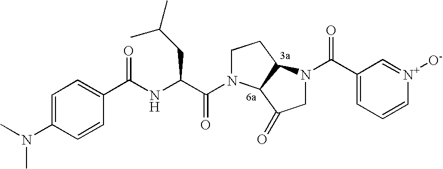

HPLC Rt=4.7 mins (>95%), HPLC-MS 508.2 [M+H]⁺, 526.2 [M+H+H₂O]⁺.

Oxidation of the intermediate was performed as detailed in the general soild phase methods prior to compound release from the solid phase.

### EXAMPLE 82

### (3aR,6aS)-4-Dimethylamino-N-{(1S)-3-methyl-1-[6-oxo-4-(pyridinecarbonyl)-hexahydro-pyrrolo[3,2-b]pyrrole-1-carbonyl]-butyl}-benzamide

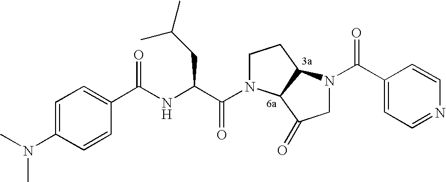

HPLC Rt=4.5 mins (>95%), HPLC-MS 492.2 [M+H]⁺, 510.2 [M+H+H₂O]⁺.

### EXAMPLE 83

### (3aR,6aS)-4-Dimethylamino-N-{(1S)-3-methyl-1-[6-oxo-4-(1-oxy-pyridine-4-carbonyl)-hexahydro-pyrrolo[3,2-b]pyrrole-1-carbonyl]-butyl}-benzamide

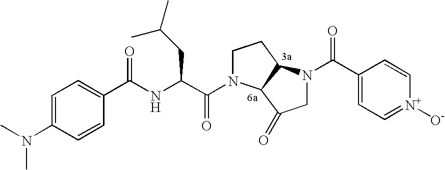

HPLC Rt=4.9 mins (>95%), HPLC-MS 508.2 [M+H]⁺, 526.2 [M+H+H₂O]⁺.

Oxidation of the intermediate was performed as detailed in the general soild phase methods prior to compound release from the solid phase.

### EXAMPLE 84

### (3aR,6aS)-1-(4-Trifluoromethyl-pyrimidin-2-yl)-piperidine-4-carboxylic acid [(1S)-1-(4-benzoyl-6-oxo-hexahydro-pyrrolo[3,2-b]pyrrole-1-carbonyl)-3-methyl-butyl]-amide

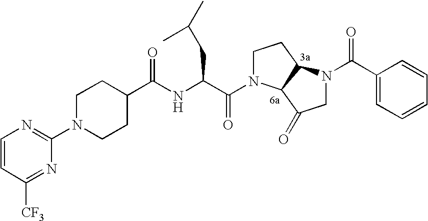

HPLC Rt=16.49 mins (>95%), HPLC-MS 601.2 [M+H]⁺.

### EXAMPLE 85

### (3aR,6aS)-N-[(1S)-1-(4-Benzoyl-6-oxo-hexahydro-pyrrolo[3,2-b]pyrrole-1-carbonyl)-3-methyl-butyl]-4-morpholin-4-yl-benzamide

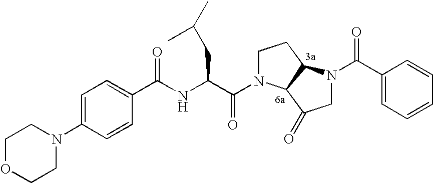

HPLC Rt=9.37 mins (>95%), HPLC-MS 533.2 [M+H]⁺.

### EXAMPLE 86

### (3aR,6aS)-Quinoline-6-carboxylic acid [(1S)-1-(4-benzoyl-6-oxo-hexahydro-pyrrolo[3,2-b]pyrrole-1-carbonyl)-3-methyl-butyl]-amide

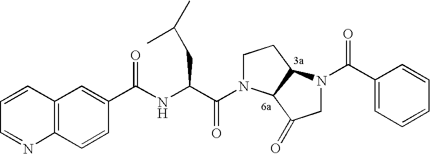

HPLC Rt=11.73 mins (>95%), HPLC-MS 499.2 [M+H]⁺.

### EXAMPLE 87

### (3aR,6aS)-1,2,3,4-Tetrahydro-quinoline-6-carboxylic acid [(1S)-1-(4-benzoyl-6-oxo-hexahydro-pyrrolo[3,2-b]pyrrole-1-carbonyl)-3-methyl-butyl]-amide

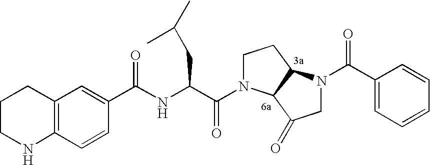

HPLC Rt=9.06 mins (>95%), HPLC-MS 503.2 [M+H]⁺, 521.2 [M+H+H₂O]⁺.

### EXAMPLE 88

### (3aR,6aS)-1H-Indole-6-carboxylic acid [(1S)-1-(4-benzoyl-6-oxo-hexahydro-pyrrolo[3,2-b]pyrrole-1-carbonyl)-3-methyl-butyl]-amide

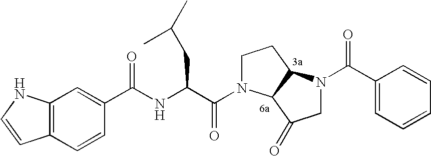

HPLC Rt=10. 13 mins (>85%), HPLC-MS 487.1 [M+H]⁺.

### EXAMPLE 89

### (3aR,6aS)-1H-Indole-5-carboxylic acid [(1S)-1-(4-benzoyl-6-oxo-hexahydro-pyrrolo[3,2-b]pyrrole-1-carbonyl)-3-methyl-butyl]-amide

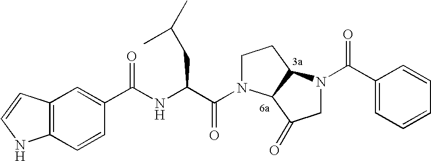

HPLC Rt=9.58 mins (>85%), HPLC-MS 487.2 [M+H]⁺.

### EXAMPLE 90

### (3aR,6aS)-Benzothiazole-6-carboxylic acid [(1S)-1-(4-benzoyl-6-oxo-hexahydro-pyrrolo[3,2-b]pyrrole-1-carbonyl)-3-methyl-butyl]-amide

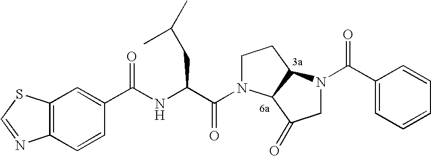

HPLC Rt=14.15 mins (>95%), HPLC-MS 505.1 [M+H]⁺.

### EXAMPLE 91

### (3aR,6aS)-N-[(1S)-1-(4-Benzoyl-6-oxo-hexahydro-pyrrolo[3,2-b]pyrrole-1-carbonyl)-3-methyl-butyl]-4-pyrazol-1-yl-benzamide

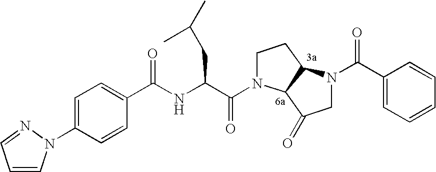

HPLC Rt=10.25 mins (>95%), HPLC-MS 514.2 [M+H]⁺.

### EXAMPLE 92

### (3aR,6aS)-3-Aminomethyl-N-[(1S)-1-(4-benzoyl-6-oxo-hexahydro-pyrrolo[3,2-b]pyrrole-1-carbonyl)-3-methyl-butyl]-benzamide

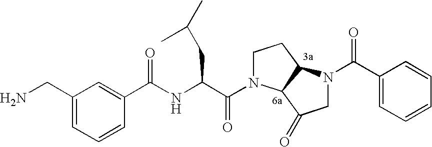

HPLC Rt=7.42 mins (>95%), HPLC-MS 477.2 [M+H]⁺, 495.2 [M+H+H₂O]⁺, 975.3 [2M+Na]⁺.

### EXAMPLE 93

### (3aR,6aS)-4-Aminomethyl-N-[(1S)-1-(4-benzoyl-6-oxo-hexahydro-pyrrolo[3,2-b]pyrrole-1-carbonyl)-3-methyl-butyl]-benzamide

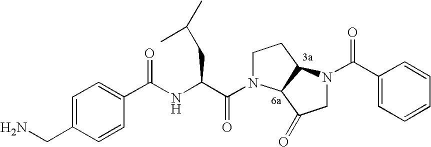

HPLC Rt=6.86 mins (>95%), HPLC-MS 477.2 (M+H)⁺, 495.2 [M+H+H₂O]⁺, 975.3 [2M+Na]⁺.

### EXAMPLE 94

### (3aR,6aS)-4-Dimethylamino-N-{(1S)-3-methyl-1-[4-(6-morpholin-4-yl-pyridine-3-carbonyl)-6-oxo-hexahydro-pyrrolo[3,2-b]pyrrole-1-carbonyl]-butyl}-benzamide

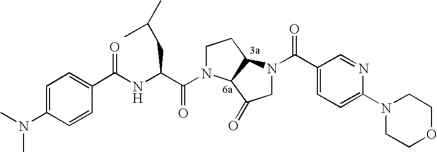

HPLC Rt=4.4 mins (>90%), HPLC-MS 577.2 [M+H]⁺.

### EXAMPLE 95

### (3aR,6aS)-4-Dimethylamino-N-{(1S)-3-methyl-1-[6-oxo-4-(thiophene-2-carbonyl)-hexahydro-pyrrolo[3,2-b]pyrrole-1-carbonyl]-butyl}-benzamide

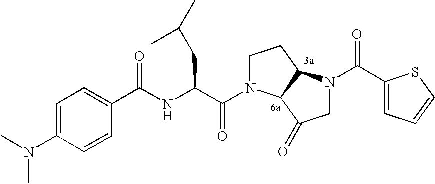

HPLC Rt=7.7 mins (>90%), HPLC-MS 497.1 [M+H]⁺.

### EXAMPLE 96

### (3aR,6aS)-4-Dimethylamino-N-{(1S)-1-[4-(furan-2-carbonyl)-6-oxo-hexahydro-pyrrolo[3,2-b]pyrrole-1-carbonyl]-3-methyl-butyl}-benzamide

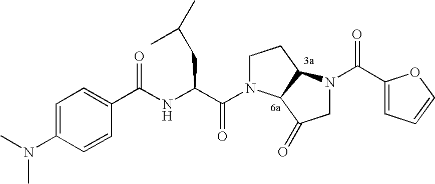

HPLC Rt=7.3 mins (>90%), HPLC-MS 481.2 [M+H]⁺, 499.2 [M+H+H₂O]⁺, 983.3 [2M+Na]⁺.

### EXAMPLE 97

### (3aR,6aS)-4-Dimethylamino-N-{(1S)-1-[4-(furan-3-carbonyl)-6-oxo-hexahydro-pyrrolo[3,2-b]pyrrole-1-carbonyl]-3-methyl-butyl}-benzamide

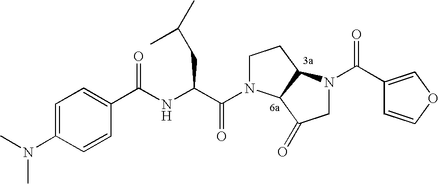

HPLC Rt=7.30 mins (>90%), HPLC-MS 481.2 [M+H]⁺, 499.2 [M+H+H₂O]⁺, 983.3 [2M+Na]⁺.

### EXAMPLE 98

### (3aR,6aS)-4-Dimethylamino-N-{(1S)-1-[4-(1H-indole-5-carbonyl)-6-oxo-hexahydro-pyrrolo[3,2-b]pyrrole-1-carbonyl]-3-methyl-butyl}-benzamide

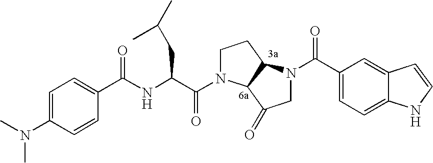

HPLC Rt=9.0 mins (>80%), HPLC-MS 530.2 [M+H]⁺.

### EXAMPLE 99

### (3aR,6aS)-4-Dimethylamino-N-{(1S)-1-[4-(1H-indole-6-carbonyl)-6-oxo-hexahydro-pyrrolo[3,2-b]pyrrole-1-carbonyl]-3-methyl-butyl}-benzamide

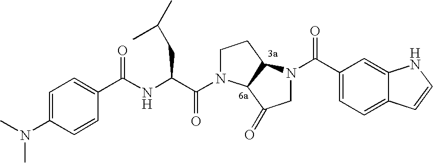

HPLC Rt=7.8 mins (>80%), HPLC-MS 530.2 [M+H]⁺.

### EXAMPLE 100

### (3aR,6aS)-N-{(1S)-1-[4-(Benzo[b]thiophene-2-carbonyl)-6-oxo-hexahydro-pyrrolo[3,2-b]pyrrole-1-carbonyl)-3-methyl-butyl}-4-dimethylamino-benzamide

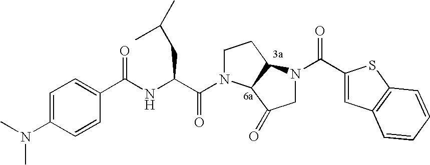

HPLC Rt=12.85 mins (>95%), HPLC-MS 547.2 [M+H]⁺.

### EXAMPLE 101

### (3aR,6aS)-N-{(1S)-1-[4-(Benzofuran-2-carbonyl)-6-oxo-hexahydro-pyrrolo[3,2-b]pyrrole-1-carbonyl]-3-methyl-butyl}-4-dimethylamino-benzamide

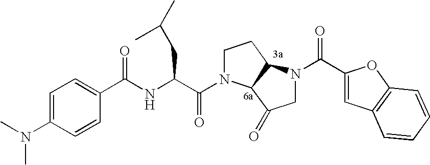

HPLC Rt=11.9 mins (>95%), HPLC-MS 531.2 [M+H]⁺.

### EXAMPLE 102

### (3aR,6aS)-N-{(1S)-1-[4-(Benzo[1,2,5]oxadiazole-5-carbonyl)-6-oxo-hexahydro-pyrrolo[3,2-b]pyrrole-1-carbonyl]-3-methyl-butyl}-4-dimethylamino-benzamide

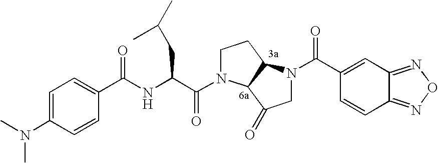

HPLC Rt=7.06 mins (>90%), HPLC-MS 533.2 [M+H]⁺, 551.2 [M+H+H₂O]⁺.

### EXAMPLE 103

### (3aR,6aS)-N-{(1S)-1-[4-(Benzo[b]thiophene-3-carbonyl)-6-oxo-hexahydro-pyrrolo[3,2-b]pyrrole-1-carbonyl]-3-methyl-butyl}-4-dimethylamino-benzamide

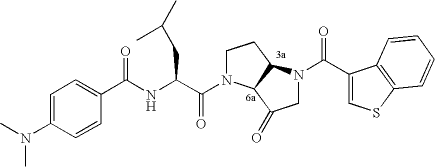

HPLC Rt=11.8 mins (>95%), HPLC-MS 547.2 [M+H]⁺.

### EXAMPLE 104

### (3aR,6aS)-N-[(1S)-1-(4-Cyclohexanecarbonyl-6-oxo-hexahydro-pyrrolo[3,2-b]pyrrole-1-carbonyl)-3-methyl-butyl]-4-dimethylamino-benzamide

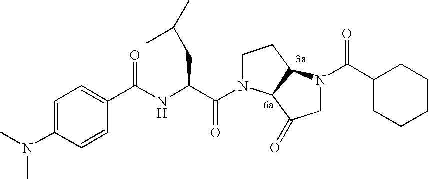

HPLC Rt=12.9-13.9 mins (>90%), HPLC-MS 497.2 [M+H]⁺, 515.2 [M+H+H₂O]⁺.

### EXAMPLE 105

### (3aR,6aS)-4-Dimethylamino-N-{(1S)-3-methyl-1-[4-(4-methyl-pentanoyl)-6-oxo-hexahydro-pyrrolo[3,2-b]pyrrole-1-carbonyl]-butyl}-benzamide

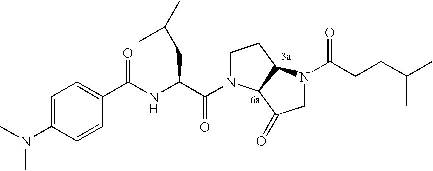

HPLC Rt=13.08 mins (>90%), HPLC-MS 485.2 [M+H]⁺, 503.2 [M+H+H₂O]⁺, 991.4 [2M+Na]⁺,

### EXAMPLE 106

### (3aS,6aR)-{4-[(2S)-2-(4-Dimethylamino-benzoylamino)-4-methyl-pentanoyl]-3-oxo-hexahydro-pyrrolo[3,2-b]pyrrol-1-yl}-4-oxo-butyric acid

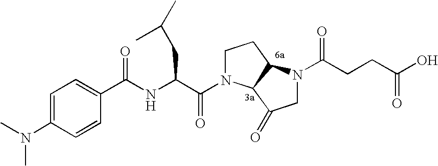

HPLC Rt=4.35 mins (>90%), HPLC-MS 487.2 [M+H]⁺.

### EXAMPLE 107

### (3aS,6aR)-5-{4-[(2S)-2-(4-Dimethylamino-benzoylamino)-4-methyl-pentanoyl]-3-oxo-hexahydro-pyrrolo[3,2-b]pyrrole-1-yl}-5-oxo-pentanoic acid

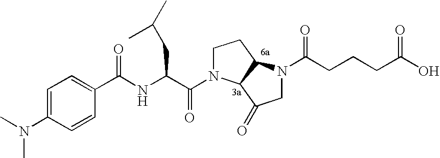

HPLC Rt=4.99 mins (>90%), HPLC-MS 501.2 [M+H]⁺.

### EXAMPLE 108

### (3aR,6aS)-N-{(1S)-1-[4-(3-Amino-propionyl)-6-oxo-hexahydro-pyrrolo[3,2-b]pyrrole-1-carbonyl]-3-methyl-butyl}-4-dimethylamino-benzamide

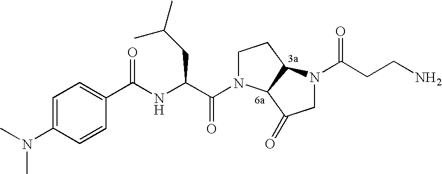

HPLC Rt=3.9 mins (>90%), HPLC-MS 458.2 [M+H]⁺, 915.4 [2M+H]⁺.

### EXAMPLE 109

### (3aS,6aR)-((1R)-1-{4[(2S)-2-(4-Dimethylamino-benzoylamino)-4-methyl-pentanoyl]-3-oxo-hexahydro-pyrrolo[3,2-b]pyrrole-1-carbonyl}-3-methyl-butyl)-carbamic acid benzyl ester

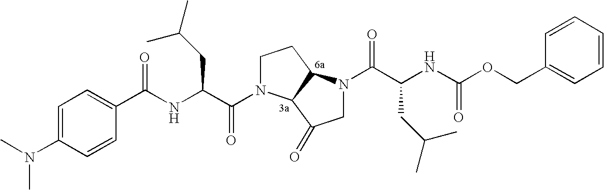

HPLC Rt=13.0-14.2 mins (>90%), HPLC-MS 634.3 [M+H]⁺.

### EXAMPLE 110

### (3aR,6aS)-N-[(1S)-1-(4-Acetyl-6-oxo-hexahydro-pyrrolo[3,2-b]pyrrole-1-carbonyl)-3-methyl-butyl]-4-dimethylamino-benzamide

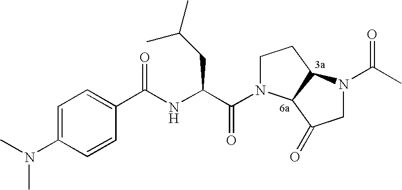

HPLC Rt=4.58 mins (>90%), HPLC-MS 429.2 [M+H]⁺, 451.2 [M+Na]⁺.

### EXAMPLE 111

### (3aR,6aS)-4-Dimethylamino-N-{(1S)-1-[4-(3,5-dimethyl-benzoyl)-6-oxo-hexahydro-pyrrolo[3,2-b]pyrrole-1-carbonyl]-3-methyl-butyl}-benzamide

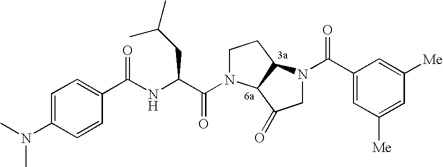

HPLC Rt=15.53 mins (>90%), HPLC-MS 519.3 [M+H]⁺.

### EXAMPLE 112

### (3aR,6aS)-N-{(1S)-1-[4-(3,5-Dimethoxy-benzoyl)-6-oxo-hexahydro pyrrolo[3,2-b]pyrrole-1-carbonyl]-3-methyl-butyl}-4-dimethylamino-benzamide

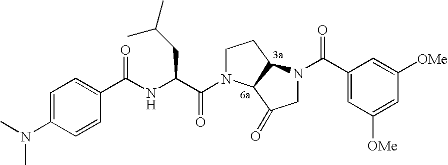 HPLC Rt=14.43 mins (>90%), HPLC-MS 551.2 [M+H]⁺.

### EXAMPLE 113

### (3aR,6aS)-4-Dimethylamino-N-{(1S)-1-[4-(3-fluoromethyl-benzoyl)-6-oxo-hexahydro-pyrrolo[3,2-b]pyrrole-1-carbonyl]-3-methyl-butyl}-benzamide

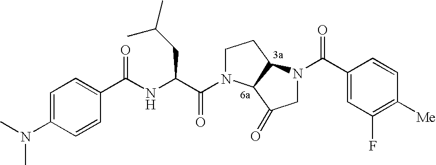

HPLC Rt=15.04 mins (>95%), HPLC-MS 523.2 [M+H]⁺, 541.2 [M+H+H₂O]⁺.

### EXAMPLE 114

### (3aR,6aS)-4-Dimethylamino-N-{(1S)-1-[4-(3,4-dimethyl-benzoyl)-6-oxo-hexahydro-pyrrolo[3,2-b]pyrrole-1-carbonyl]-3-methyl-butyl}-benzamide

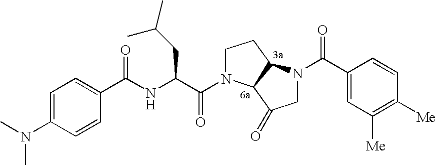

HPLC Rt=15.70 mins (>90%), HPLC-MS 519.3 [M+H]⁺.

### EXAMPLE 115

### (3aR,6aS)-4-Dimethylamino-N-{(1S)-1-[4-(4-fluoro-3-methyl-benzoyl)-6-oxo-hexahydro-pyrrolo[3,2-b]pyrrole-1-carbonyl]-3-methyl-butyl}-benzamide

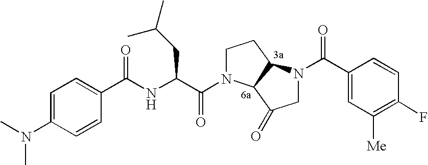

HPLC Rt=15.00 mins (>90%), HPLC-MS 523.2 [M+H]⁺, 541.2 [M+H+H₂O]⁺.

### EXAMPLE 116

### (3aR,6aS)-N-{(1S)-1-[4-(3,4-Difluoro-benzoyl)-6-oxo-hexahydro-pyrrolo[3,2-b]pyrrole-1-carbonyl]-3-methyl-butyl}-4-dimethylamino-benzamide

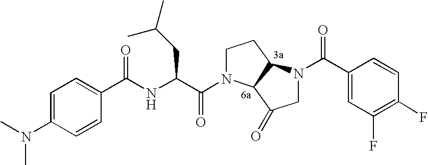

HPLC Rt=14.47 mins (>90%), HPLC-MS 527.2 [M+H]⁺, 545.2 [M+H+H₂O]⁺.

### EXAMPLE 117

### (3aR,6aS)-N-[(1S)-1-(4-Benzoyl-6-oxo-hexahydro-pyrrolo[3,2-b]pyrrole-1-carbonyl)-3-methyl-butyl]-4-(N-oxy-dimethylamino)-benzamide

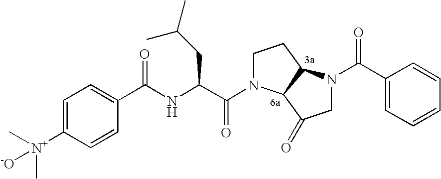

HPLC Rt=11.64 mins (>95%), HPLC-MS 507.2 [M+H]⁺, 525.2 [M+H+H₂O]⁺.

Oxidation of the intermediate was performed as detailed in the general soild phase methods prior to compound release from the solid phase.

### EXAMPLE 118

### (3aR,6aS)-3-Aminomethyl-N-[(1S)-3-methyl-1-(6-oxo-4-phenylacetyl-hexahydro-pyrrolo[3,2-b]pyrrole-1-carbonyl)-butyl]-benzamide

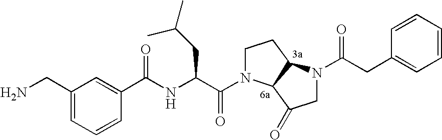

HPLC Rt=12.06 mins (>90%), HPLC-MS 491.2 [M+H]⁺, 509.2 [M+H+H₂O]⁺, 981.4 [2M+H]⁺.

### EXAMPLE 119

### (3aR,6aS)-N-[(1S)-1-(4-Benzenesulfonyl-6-oxo-hexahydro-pyrrolo[3,2-b]pyrrole-1-carbonyl)-3-methyl-butyl]-4-tert-butyl-benzamide

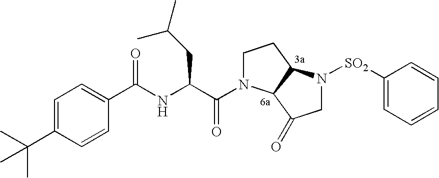

HPLC Rt=21.0-22.4 mins (>75%), HPLC-MS 540.1 [M+H]⁺.

EXAMPLES 119-123 were prepared following the general methods detailed for EXAMPLE 1, but using an alternative building block (3aR,6aS)-4-benzenesulfonyl-6-oxo-hexahydropyrrolo [3,2-b]pyrrole-1-carboxylic acid 9H-fluoren-9-ylmethyl ester (84) prepared following Scheme 21.

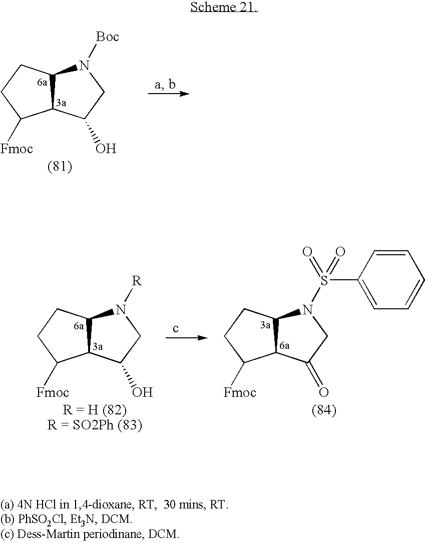

### Preparation of (3aR,6S,6aS)-6-hydroxyhexahydropyrrolo[3,2-b]pyrrole-1-carboxylic acid 9H-fluoren-9-ylmethyl ester hydrochloride (82)

A solution of HCl in 1,4-dioxane (4.0M, 2.0 ml, 8 mmol) was added to (3S,3aS,6aR)-3-hydroxyhexahydropyrrolo[3,2-b]pyrrole-1,4-dicarboxylic acid 1-tert-butyl ester 4-(9H-fluoren-9-ylmethyl) ester (81) (65 mg, 0.14 mmol). The mixture was stirred in a sealed system for 50 minutes then the solvents were removed in vacuo to leave a residue which was azeotroped with diethyl ether (3×10 ml) to obtain (3aR,6S,6aS)-6-hydroxyhexahydropyrrolo[3,2-b]pyrrole-1-carboxylic acid 9H-fluoren-9-ylmethyl ester hydrochloride (82) as a white solid which was used without further purification (see below). HPLC-MS 351.1 [M+H]⁺, 373.1 [M+Na]⁺, 723.2 [2M+Na]⁺; HRMS C₂₁H₂₃N₂O₃Na req. 351.1708, fnd. 351.1712 (0.95 ppm).

### Preparation of (3aR,6S,6aS)-4-benzenesulfonyl-4-hydroxyhexahydropyrrolo [3,2-b]pyrrole-1-carboxylic acid 9H-fluoren-9-ylmethyl ester (83)

Dichloromethane (1.5 ml), benzenesulfonyl chloride (20 μl, 0.16 mmol) then triethylamine (44 μl, 0.32 mmol) were added consecutively whilst stirring to (3aR,6S,6aS)-6-hydroxyhexahydropyrrolo[3,2-b]pyrrole-1-carboxylic acid 9H-fluoren-9-ylmethyl ester hydrochloride (82) (prepared as above, 0.14 mmol) under an atmosphere of argon. The mixture was stirred for 1 hour then the product extracted into ethyl acetate (40 ml), washed with aqueous saturated sodium hydrogen carbonate (40 ml), pH 3 hydrochloric acid (40 ml) and brine (40 ml) then dried (Na₂SO₄), and the solvents removed in vacuo. The residue was purified by flash chromatography over silica eluting with ethyl acetate:heptane mixtures 0:100 to 30:70 to give (3aR,6S,6aS)-4-benzenesulfonyl-6-hydroxyhexahydropyrrolo[3,2-b]pyrrole-1-carboxylic acid 9H-fluoren-9-ylmethyl ester (83) as a white solid (60 mg, 86%). TLC (Single spot, R_{f}=0.40, EtOAc:heptane 1:1), analytical HPLC single main peak Rₜ=20.112 min; HPLC-MS 491.0 [M+H]⁺, 513.0 [M+Na]⁺; Elemental analysis C₂₇H₂₆N₂O₅S req. (fnd.) % C 66.10 (66.02), % H 5.34 (5.36), % N 5.71 (5.61); HRMS C₂₇H₂₆N₂O₅SNa req. 513.1460, fnd. 513.1489 (5.56 ppm).

### Preparation of (3aR,6aS)-4-benzenesulfonyl-6-oxo-hexahydropyrrolo[3,2-b]pyrrole-1-carboxylic acid 9H-fluoren-9-ylmethyl ester (84)

Dess-Martin periodinane (95 mg, 0.22 mmol) was added in portions to a stirred solution of (3aR,6S,6aS)-4-benzenesulfonyl-6-hydroxyhexahydropyrrolo[3,2-b]pyrrole-1-carboxylic acid 9H-fluoren-9-yl methyl ester (83) (55 mg, 0.11 mmol) in dichloromethane (1.5 ml) under an atmosphere of argon over 2 minutes. The mixture was stirred for 3.25 hours then the solvents removed in vacuo to obtain a residue which was purified by flash chromatography over silica eluting with ethyl acetate:heptane mixtures 0:100 to 25:75 to give (3aR,6aS)-4-benzenesulfonyl-6-oxo-hexahydropyrrolo[3,2-b]pyrrole-1-carboxylic acid 9H-fluoren-9-ylmethyl ester (84) as a white solid (44 mg, 82%). TLC (Single spot, R_{f}=0.55, EtOAc:heptane 65:35), analytical HPLC broad peak Rₜ=20.0-21.5 min; HPLC-MS single broad main UV peak, 489.0 [M+H]⁺, 511.0 [M+Na]⁺, 529.0 [M+H₂O+Na]⁺, 999.0 [2M+Na]⁺; C₂₇H₂₄N₂O₅S.0.4CDCl₃ req. (fnd.) % C 61.36 (61.09), % H 4.51 (4.76), % N 5.22 (4.79); HRMS C₂₁H₂₄N₂O₅SNa req. 511.1304, fnd. 511.1615 (2.16 ppm); d_{H} (500 MHz, CDCl₃) mixture of rotamers 2.10-2.28 (2H, m, PhSO₂NCHCH₂), 3.40-3.60 (2H, m, FmocNCH₂), 3.62-3.84 (2H, m, PhSO₂NCH₂), 4.16-4.46 (4H, m, FmocNCH, Fmoc-CH and Fmoc-CH₂), 4.48-4.61 (1H, m, PhSO₂NCR), 7.32-7.90 (13H, m, aromatic); d_{C} (125 MHz, CDCl₃) 31.72, 31.86 (PhSO₂NCHCH₂), 45.41 (FmocNCH₂), 47.15 (Fmoc-CH), 52.62 (PhSO₂NCH₂), 60.17 (PhSO₂NCH), 63.30, 63.52 (FmocNCH), 67.79, 68.12 (Fmoc-CH₂), 119.97, 120.09, 124.94, 127.07, 127.53, 127.74, 127.91, 129.65, 133.75 (aromatic CH), 141.29, 143.40, 143.58, 143.81, 144.12 (quaternary aromatic), 154.93 (NC═O), 203.85, 204.07 (C═O).

Following the general details from Scheme 6, the required bicycle building block (3aR,6aS)-4-benzenesulfonyl-6-oxo-hexahydropyrrolo[3,2-b]pyrrole-1-carboxylic acid 9H-fluoren-9-ylmethyl ester (84) was converted to building block-linker construct (27) (where Pg₂ is phenylsulphonyl) as follows:
A solution of sodium acetate trihydrate (30 mg, 0.221 mmol) in water (0.3 ml) was added to a solution of (3aR,6aS)-1-benzenesulfonyl-6-oxo-hexahydropyrrolo [3,2-b]pyrrole-1-carboxylic acid 9H-fluoren-9-ylmethyl ester (84) (36 mg, 0.074 mmol) and 4-[[(hydrazinocarbonyl)amino]methyl]cyclohexane carboxylic acid. trifluoroacetate (Murphy, A. M., et al, J. Am. Chem. Soc, 114, 3156-3157, 1992) (49 mg, 0.148 mmol) in ethanol (2.1 ml). The reaction heated at 75° C. in a sealed tube for 4.5 hour. The product was extracted into chloroform (50 ml) then washed with hydrochloric acid (0.1M, 2×25 ml), saturated aqueous sodium chloride solution (30 ml) then dried (Na₂SO₄) and the solvent removed in vacuo to leave the product as a white solid (46 mg, 91%). Analytical HPLC has main UV peaks with Rt=19.624 and 21.252 mins and HPLC-MS (main UV peaks each with 686.3 [M+H]⁺).

Following the general details from Scheme 6, the required building block-linker construct (27) was attached to the solid phase providing loaded building block-linker construct (28) following standard loading protocols and indicated quantitative loading.

EXAMPLES 120 to 123 were prepared as detailed for EXAMPLE 119, substituting the appropriate carboxylic acids as required;

### EXAMPLE 120

### (3aR,6aS)-N-[(1S)-1-(4-Benzenesulfonyl-6-oxo-hexahydropyrrolo [3,2-b]pyrrole-1-carbonyl)-3-methyl-butyl]-4-thiophen-2-yl-benzamide

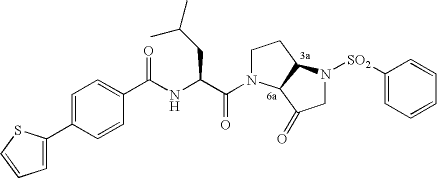

HPLC Rt=20.3-21.7 mins (>75%), HPLC-MS 566.1 [M+H]⁺.

### EXAMPLE 121

### (3aR,6aS)-Benzofuran-2-carboxylic acid [(1S)-1-(4-benzene sulfonyl-6-oxo-hexahydro-pyrrolo[3,2-b]pyrrole-1-carbonyl)-3-methyl-butyl]-amide

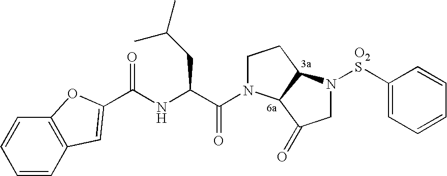

HPLC Rt=18.3-19.9 mins (>95%), HPLC-MS 524.1 [M+H]⁺, 546.2 [M+Na]⁺.

### EXAMPLE 122

### (3aR,6aS)-N-[(1S)-1-(4-Benzenesulfonyl-6-oxo-hexahydropyrrolo [3,2-b]pyrrole-1-carbonyl)-3-methyl-butyl]-4-morpholin-4-yl-benzamide

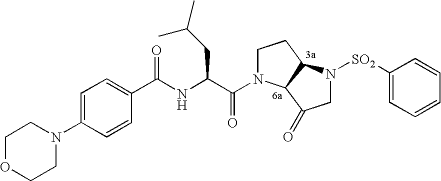

HPLC Rt=16.4-17.8 mins (>90%), HPLC-MS 569.1 [M+H]⁺, 587.1 [M+H+H₂O]⁺.

### EXAMPLE 123

### (3aR,6aS)-3-Aminomethyl-N-[(1S)-1-(4-benzenesulfonyl-6-oxo-hexahydro-pyrrolo[3,2-b]pyrrole-1-carbonyl)-3-methyl-butyl]-benzamide

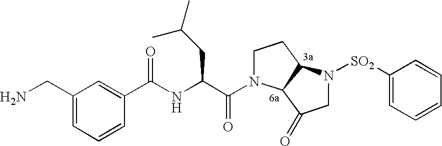

HPLC Rt=13.0-14.1 mins (>90%), HPLC-MS 513.1 [M+H]⁺, 531.1 [M+H+H₂O]⁺.

The following examples were prepared as detailed for EXAMPLE 1, substituting the appropriate carboxylic acids as required;

### EXAMPLE 124

### (3aR,6aS)-4-Dimethylamino-N-{(1S)-3-methyl-1-[6-oxo-4-(3,3,3-trifluoro-propionyl)-hexahydro-pyrrolo[3,2-b]pyrrole-1-carbonyl]-butyl}-benzamide

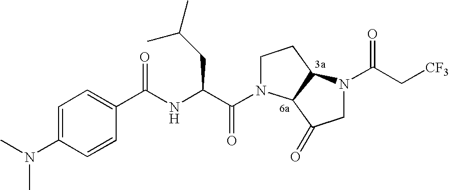

HPLC Rt=10.46 mins (>95%), HPLC-MS 497.2 [M+H]⁺, 515.2 [M+H+H₂O]⁺.

### EXAMPLE 125

### (3aR,6aS)-N-{(1S)-1-[4-(2,2-Difluoro-acetyl)-6-oxo-hexahydro-pyrrolo[3,2-b]pyrrole-1-carbonyl]-3-methyl-butyl}-4-dimethylamino-benzamide

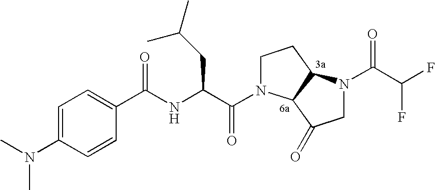

HPLC Rt=9.31 mins (>60%), HPLC-MS 465.1 [M+H]⁺, 483.1 [M+H+H₂O]⁺.

### EXAMPLE 126

### (3aR,6aS)-4-Dimethylamino-N-[(1S)-3-methyl-1-(6-oxo-4-propionyl-hexahydro-pyrrolo[3,2-b]pyrrole-1-carbonyl)-butyl]-benzamide

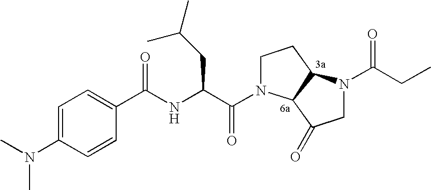

HPLC Rt=9.90 mins (>95%), HPLC-MS 443.2 [M+H]⁺, 461.2 [M+H+H₂O]⁺, 907.3 [2M+Na]⁺.

### EXAMPLE 127

### (3aR,6aS)-N-[(1S)-1-(4-Butyryl-6-oxo-hexahydro-pyrrolo[3,2-b]pyrrole-1-carbonyl)-3-methyl-butyl]-4-dimethylamino-benzamide

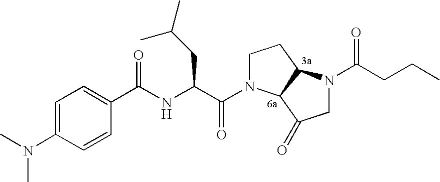

HPLC Rt=10.95 mins (>90%), HPLC-MS 457.2 [M+H]⁺, 475.2 [M+H+H₂O]⁺, 935.3 [2M+Na]⁺.

### EXAMPLE 128

### (3aR,6aS)-4-Dimethylamino-N-[(1S)-3-methyl-1-(6-oxo-4-pentanoyl-hexahydro-pyrrolo[3,2-b]pyrrole-1-carbonyl)-butyl]-benzamide

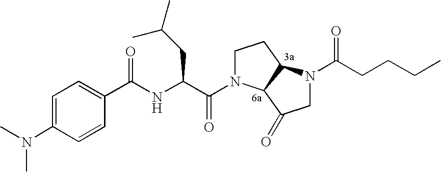

HPLC Rt=12.0-13.1 mins (>90%), HPLC-MS 471.2 [M+H]⁺, 489.2 (M+H+ H₂O]⁺, 963.3 [2M+Na]⁺.

### EXAMPLE 129

### (3aR,6aS)-4-Dimethylamino-N-[(1S)-1-(4-isobutyryl-6-oxo-hexahydro-pyrrolo[3,2-b]pyrrole-1-carbonyl)-3-methyl-butyl]-benzamide

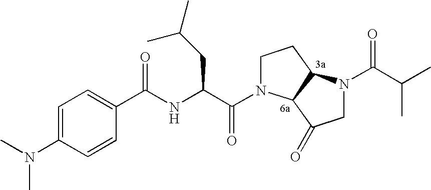

HPLC Rt=10.55 mins (>90%), HPLC-MS 457.2 [M+H]⁺, 475.2 [M+H+H₂O]⁺, 935.4 [2M+Na]⁺.

### EXAMPLE 130

### (3aR,6aS)-4-Dimethylamino-N-{(1S)-3-methyl-1-[4-(3-methyl-butyryl)-6-oxo-hexahydro-pyrrolo[3,2-b]pyrrole-1-carbonyl]-butyl}-benzamide

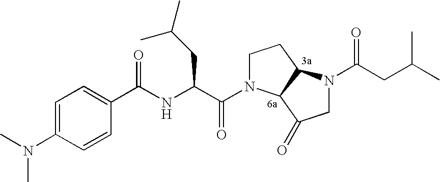

HPLC Rt=11.81 mins (>90%), HPLC-MS 471.2 [M+H]⁺, 489.2 [M+H+H₂O]⁺, 963.4 [2M+Na]⁺.

### EXAMPLE 131

### (3aR,6aS)-N-[(1S)-1-(4-Cyclopropanecarbonyl-6-oxo-hexahydro-pyrrolo[3,2-b]pyrrole-1-carbonyl)-3-methyl-butyl]-4-dimethylamino-benzamide

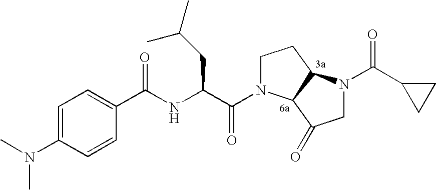

HPLC Rt=110.10 mins (>90%), HPLC-MS 455.2 [M+H]⁺, 473.2 [M+H+H₂O]⁺, 931.3 [2M+Na]⁺.

### EXAMPLE 132

### (3aR,6aS)-N-{(1S)-1-[4-(2-Cyclopropyl-acetyl)-6-oxo-hexahydro-pyrrolo[3,2-b]pyrrole-1-carbonyl]-3-methyl-butyl}-4-dimethylamino-benzamide

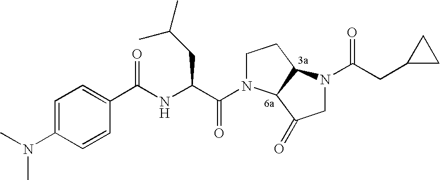

HPLC Rt=11.13 mins (>90%), HPLC-MS 469.2 [M+H]⁺, 487.2 [M+H+H₂O]⁺, 959.3 [2M+Na]⁺.

### EXAMPLE 133

### (3aR,6aS)-N-[(1S)-1-(4-Cyclobutanecarbonyl-6-oxo-hexahydro-pyrrolo[3,2-b]pyrrole-1-carbonyl)-3-methyl-butyl]-4-dimethylamino-benzamide

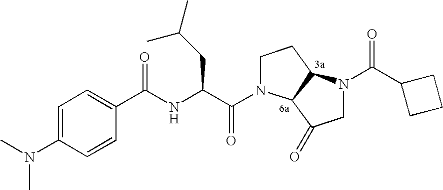

HPLC Rt=11.41 mins (>90%), HPLC-MS 469.2 [M+H]⁺, 487.2 [M+H+H₂O]⁺, 959.4 [2M+Na]⁺.

### EXAMPLE 134

### (3aR,6aS)-N-[(1S)-1-(4-Cyclopentanecarbonyl-6-oxo-hexahydro-pyrrolo[3,2-b]pyrrole-1-carbonyl)-3-methyl-butyl]-4-dimethylamino-benzamide

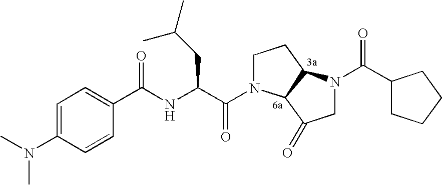

HPLC Rt=12.2-13.1 mins (>90%), HPLC-MS 483.2 [M+H]⁺, 501.2 [M+H+H₂O]⁺, 987.4 [2M+Na]⁺.

### EXAMPLE 135

### (3aR,6aS)-N-[(1S)-1-(4-Cycloheptanecarbonyl-6-oxo-hexahydro-pyrrolo[3,2-b]pyrrole-1-carbonyl)-3-methyl-butyl]-4-dimethylamino-benzamide

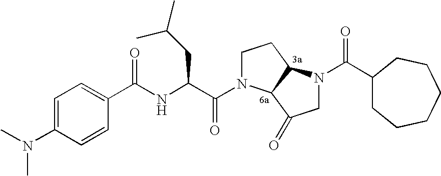

HPLC Rt=14.0-14.9 mins (>85%), HPLC-MS 511.2 [M+H]⁺, 529.3 [M+H+H₂O]⁺.

### EXAMPLE 136

### (3aR,6aS)-4-Dimethylamino-N-[(1S)-1-(4-heptanoyl-6-oxo-hexahydro-pyrrolo[3,2-b]pyrrole-1-carbonyl)-3-methyl-butyl]-benzamide

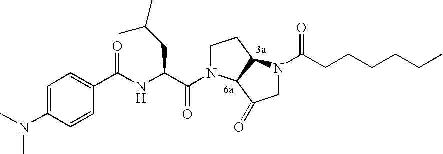

HPLC Rt=14.3-15.2 mins (>80%), HPLC-MS 499.2 [M+H]⁺, 517.2 [M+H+H₂O]⁺.

### EXAMPLE 137

### (3aR,6aS)-N-{(1S)-1-[4-(2-Cyclohexyl-acetyl)-6-oxo-hexahydro-pyrrolo[3,2-b]pyrrole-1-carbonyl]-3-methyl-butyl}-4-dimethylamino-benzamide

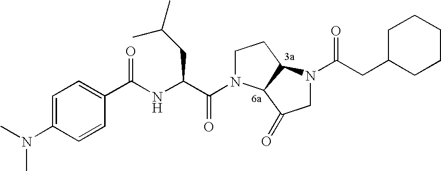

HPLC Rt=14.27 mins (>80%), HPLC-MS 511.2 [M+H]⁺, 529.3 [M+H+H₂O]⁺.

### EXAMPLE 138

### (3aR,6aS)-N-{(1S)-1-[4-(Cyclohex-3-enecarbonyl)-6-oxo-hexahydro-pyrrolo[3,2-b]pyrrole-1-carbonyl]-3-methyl-butyl}-4-dimethylamino-benzamide

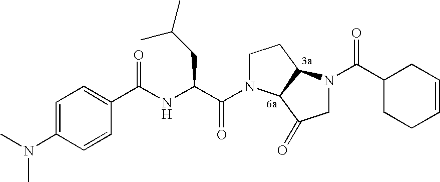

HPLC Rt=12.2-13.3 mins (>80%), HPLC-MS 495.2 [M+H]⁺, 513.2 [M+H+H₂O]⁺.

### EXAMPLE 139

### (3aR,6aS)-4-Dimethylamino-N-{(1S)-1-[4-(3-methoxy-cyclohexanecarbonyl)-6-oxo-hexahydro-pyrrolo[3,2-b]pyrrole-1-carbonyl]-3-methyl-butyl}-benzamide

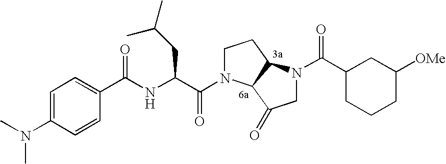

HPLC Rt=11.1-12.8 mins (>90%), HPLC-MS 527.2 [M+H]⁺, 545.3 [M+H+H₂O]⁺.

### EXAMPLE 140

### (3aR,6aS)-4-Dimethylamino-N-{(1S)-3-methyl-1-[6-oxo-4-(piperidine-3-carbonyl)-hexahydro-pyrrolo[3,2-b]pyrrole-1-carbonyl]-butyl}-benzamide

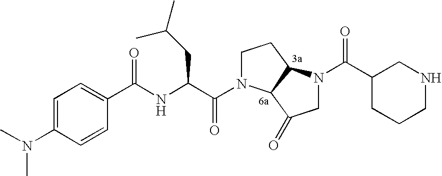

HPLC Rt=7.8-9.3 mins (>75%), HPLC-MS 498.2 [M+H]⁺, 516.2 [M+H+H₂O]⁺, 995.5 [2M+H]⁺.

### EXAMPLE 141

### (3aR,6aS)-4-Dimethylamino-N-{(1S)-3-methyl-1-[4-(3-methyl-pentanoyl)-6-oxo-hexahydro-pyrrolo[3,2-b]pyrrole-1-carbonyl]-butyl}-benzamide

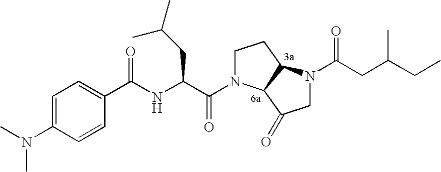

HPLC Rt=12.9-13.8 mins (>75%), HPLC-MS 485.2 [M+H]⁺, 503.3 [M+H+H₂O]⁺, 991.5 [2M+Na]⁺.

### EXAMPLE 142

### (3aR,6aS)-4-Dimethylamino-N-{(1S)-3-methyl-1-[4-(morpholin-4-carbonyl)-6-oxo-hexahydro-pyrrolo[3,2-b]pyrrole-1-carbonyl]-butyl}-benzamide

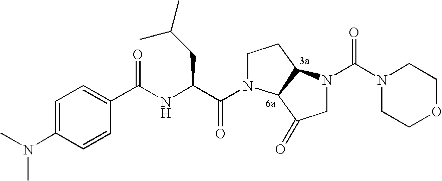

HPLC Rt=9.82 mins (>85%), HPLC-MS 500.2 [M+H]⁺.

### EXAMPLE 143

### (3aR,6aS)-N-{(1S)-1-[4-(3-Benzenesulfonyl-propionyl)-6-oxo-hexahydro-pyrrolo[3,2-b]pyrrole-1-carbonyl]-3-methyl-butyl}-4-dimethylamino-benzamide

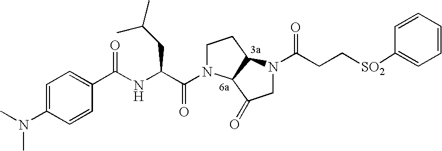

HPLC Rt=12.27 mins (>90%), HPLC-MS 583.2 [M+H]⁺.

### EXAMPLE 144

### (3aR,6aS)-4-Dimethylamino-N-{(1S)-1-[4-(2-methanesulfonyl-acetyl)-6-oxo-hexahydro-pyrrolo[3,2-b]pyrrole-1-carbonyl]-3-methyl-butyl}-benzamide

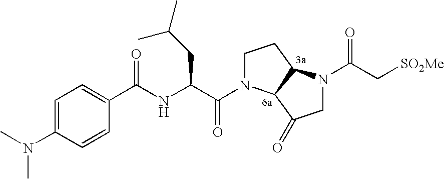

HPLC Rt=8.00 mins (>50%), HPLC-MS 507.1 [M+H]⁺.

### EXAMPLE 145

### (3aS,6aR)-((1S)-2-{4-[(2S)-2-(4-Dimethylamino-benzoylamino)-4-methyl-pentanoyl]-3-oxo-hexahydro-pyrrolo[3,2-b]pyrrole-1-yl}-1-methyl-2-oxo-ethyl)-carbamic acid benzyl ester

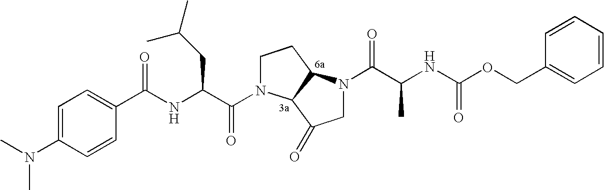

HPLC Rt=12.4-14.0 trims (>85%), HPLC-MS 592.2 [M+H]⁺.

### EXAMPLE 146

### (3aS,6aR)-((1R)-2-{4-[(2S)-2-(4-Dimethylamino-benzoylamino)-4-methyl-pentanoyl]-3-oxo-hexahydro-pyrrolo[3,2-b]pyrrol-1-yl}-1-methyl-2-oxo-ethyl)-carbamic acid benzyl ester

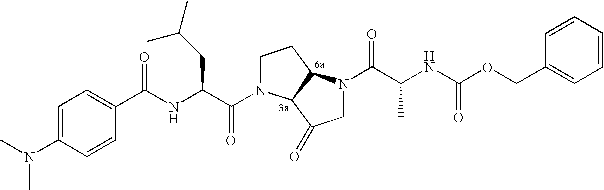

HPLC Rt=12.5-13.9 mins (>75%), HPLC-MS 592.2 [M+H]⁺.

### EXAMPLE 147

### (3aS,6aR)-((1S)-2-{4-[(2S)-2-(4-Dimethylamino-benzoylamino)-4-methyl-pentanoyl]-3-oxo-hexahydro-pyrrolo[3,2-b]pyrrole-1-yl}-1-hydroxymethyl-2-oxo-ethyl)-carbamic acid benzyl ester

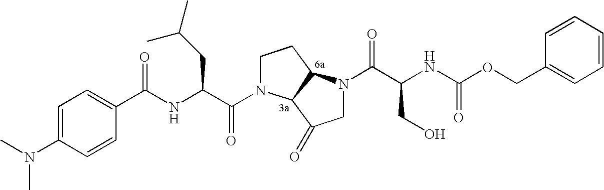

HPLC Rt=11.8-13.3 mins (>80%), HPLC-MS 608.3 [M+H]⁺.

### EXAMPLE 148

### (3aS,6aR)-((1S)-1-Carbamoylmethyl-2-{4-[(2S)-2-(4-dimethyl aminobenzoylamino)-4-methyl-pentanoyl]-3-oxo-hexahydro-pyrrolo[3,2-b]pyrrol-1-yl}-2-oxo-ethyl)-carbamic acid benzyl ester

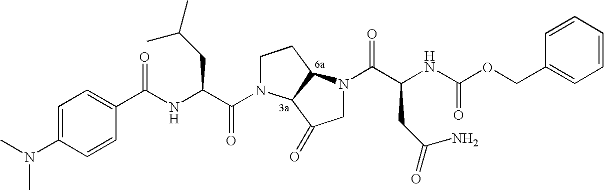

HPLC Rt=11.4-13.2 mins (>75%), HPLC-MS 635.2 [M+H]⁺.

### EXAMPLE 149

### (3aS,6aR)-(3S)-3-Benzyloxycarbonylamino-4-{4-[(2S)-2-(4-dimethylamino-benzoylamino)methyl-pentanoyl)-3-oxo-hexahydro-pyrrolo[3,2-b]pyrrole-1-yl}-4-oxo-butyric acid

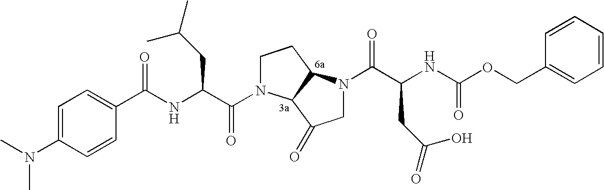

HPLC Rt=12.1-13.5 mins (>85%), HPLC-MS 636.2 [M+H]⁺, 654.3 [M+H+H₂O]⁺.

### EXAMPLE 150

### (3aR,6aS)-N-{(1S)-1-[4-((2S)-2-Amino-4-methyl-pentanoyl)-6-oxo-hexahydro-pyrrolo[3,2-b]pyrrole-1-carbonyl]-3-methyl-butyl}-4-dimethylamino-benzamide

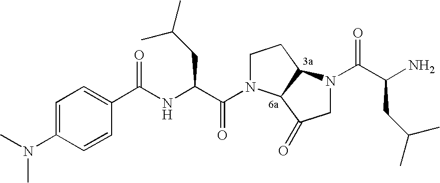

HPLC Rt=11.00 mins (>90%), HPLC-MS 500.2 [M+H]⁺, 999.5 [2M+H]⁺.

### EXAMPLE 151

### (3aR,6aS)-N-{(1S)-1-[4-((2S)-2-Acetylamino-4-methyl-pentanoyl)-6-oxo-hexahydro-pyrrolo[3,2-b]pyrrole-1-carbonyl]-3-methyl-butyl}-4-dimethylamino-benzamide

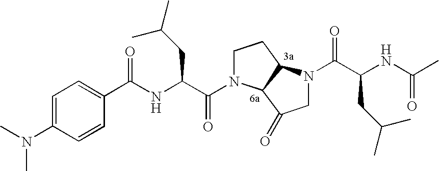

HPLC Rt=11.5-13.0 mins (>90%), HPLC-MS 542.2 [M+H]⁺.

### EXAMPLE 152

### (3aR,6aS)-N-{(1S)-1-[4-((2R)-2-Amino-4-methyl-pentanoyl)-6-oxo-hexahydro-pyrrolo[3,2-b]pyrrole-1-carbonyl]-3-methyl-butyl}-4-dimethylamino-benzamide

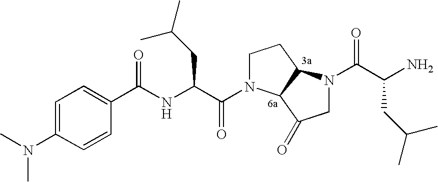

HPLC Rt=8.9-10.8 mins (>90%), HPLC-MS 500.2 [M+H]⁺, 999.5 [2M+H]⁺.

### EXAMPLE 153

### (3aR,6aS)-N-{(1S)-1-[4-((2R)-2-Acetylamino-4-methyl-pentanoyl)-6-oxo-hexahydro-pyrrolo[3,2-b]pyrrole-1-carbonyl]-3-methyl-butyl}-4-dimethylamino-benzamide

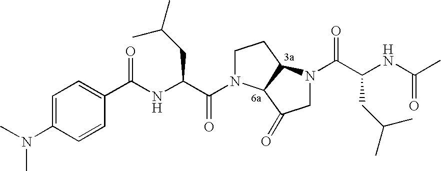

HPLC Rt=10.4-12.2 mins (>85%), HPLC-MS 542.3 [M+H]⁺.

### EXAMPLE 154

### (3aS,6aR)-(2S)-2-{4-[(2S)-2-(4-Dimethylamino-benzoylamino)-4-methyl-pentanoyl]-3-oxo-hexahydro-pyrrolo[3,2-b]pyrrole-1-carbonyl}-pyrrolidine-1-carboxylic acid benzyl ester

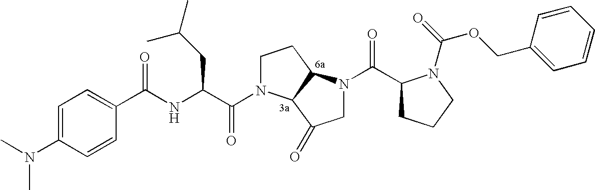

HPLC Rt=1]3.2-14.3 mins (>90%), HPLC-MS 618.2 [M+H]⁺.

### EXAMPLE 155

### (3aS,6aR)-(2R)-2-{4-[(2S)-2-(4-Dimethylamino-benzoylamino)-4-methyl-pentanoyl]-3-oxo-hexahydro-pyrrolo[3,2-b]pyrrole-1-carbonyl}-pyrrolidine-1-carboxylic acid benzyl ester

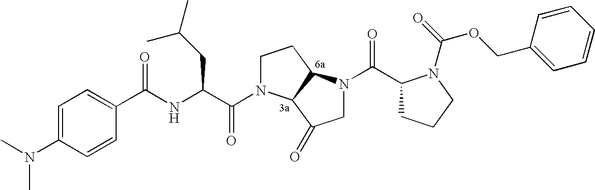

HPLC Rt=13.2-14.2 mins (>90%), HPLC-MS 618.2 [M+H]⁺.

### EXAMPLE 156

### (3aR,6aS)-4-Dimethylamino-N-{(1S)-3-methyl-1-[6-oxo-4-((2S)-pyrrolidine-2-carbonyl)-hexahydro-pyrrolo[3,2-b]pyrrole-1-carbonyl]-butyl}-benzamide

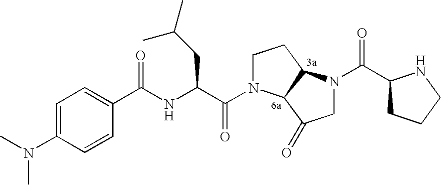

HPLC Rt=8.82 mins (>85%), HPLC-MS 484.2 [M+H]⁺, 967.4 [2M+H]⁺.

### EXAMPLE 157

### (3aR,6aS)-4-Dimethylamino-N-{(1S)-3-methyl-1-[6-oxo-4-((2R)-pyrrolidine-2-carbonyl)-hexahydro-pyrrolo[3,2-b]pyrrole-1-carbonyl]-butyl}-benzamide

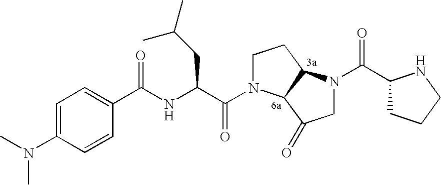

HPLC Rt=7.3-9.1 mins (>85%), HPLC-MS 484.2 [M+H]⁺, 502.2 [M+H+H₂O]⁺, 985.4 [2M+H+H₂O]⁺.

### EXAMPLE 158

### (3aS,6aR)-((S)-1-{4-[(2S)-2-(4-Dimethylamino-benzoylamino)-4-methyl-pentanoyl]-3-oxo-hexahydro-pyrrolo[3,2-b]pyrrole-1-carbonyl}-2-methyl-propyl)-carbamic acid benzyl ester

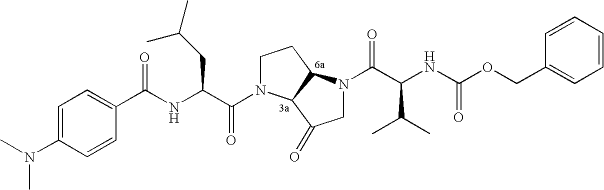

HPLC Rt=15.18 mins (>85%), HPLC-MS 620.3 [M+H]⁺.

### EXAMPLE 159

### (3aS,6aR)-((1S)-1-{4-[(2S)-2-(4-Dimethylamino-benzoylamino)-4-methyl-pentanoyl]-3-oxo-hexahydro-pyrrolo[3,2-b]pyrrole-1-carbonyl}-propyl)-carbamic acid benzyl ester

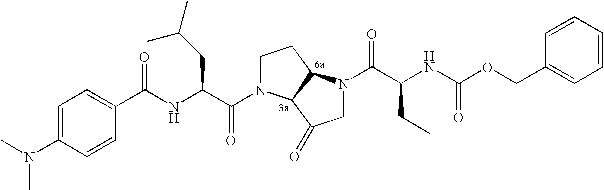

HPLC Rt=14.22 mins (>85%), HPLC-MS 606.2 [M+H]⁺.

### EXAMPLE 160

### (3aS,6aR)-((1S)-1-Benzyl-2-{-[(2S)-2-(4-dimethylamino-benzoylamino)-4-methyl-pentanoyl]-3-oxo-hexahydro-pyrrolo[3,2-b]pyrrole-1-yl}-2-oxo-ethyl)-carbamic acid benzyl ester

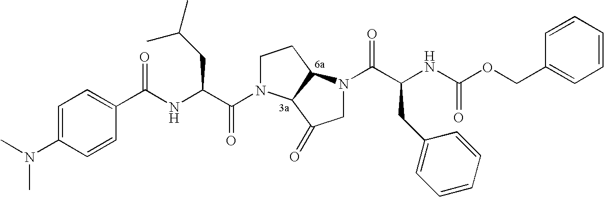

HPLC Rt=16.23 mins (>80%), HPLC-MS 668.2 [M+H]⁺.

### EXAMPLE 161

### (3 aS,6aR)-((1S,2S)-1-{4-[(2S)-2-(4-Dimethylamino-benzoylamino)-4-methyl-pentanoyl]-3-oxo-hexahydro-pyrrolo[3,2-b]pyrrole-1-carbonyl}-2-methyl-butyl)-carbamic acid benzyl ester

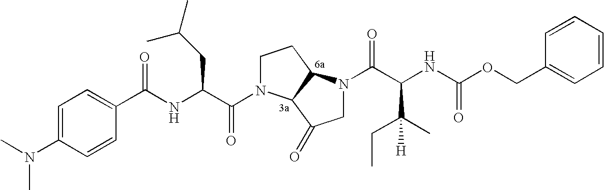

HPLC Rt=16.17 mins (>90%), HPLC-MS 634.3 [M+H]⁺.

### EXAMPLE 162

### (3aR,6aS)-N-{(1S)-1-[4-((2S)-2-Amino-propionyl)-6-oxo-hexahydro-pyrrolo[3,2-b]pyrrole-1-carbonyl]-3-methyl-butyl}-4-dimethylamino-benzamide

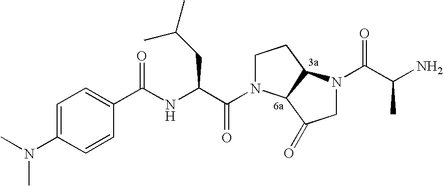

HPLC Rt=8.01 mins (>90%), HPLC-MS 458.2 [M+H]⁺, 937.4 [2M+Na]⁺.

### EXAMPLE 163

### (3aR,6aS)-N-{(1S)-1-[4-((2R)-2-Amino-propionyl)-6-oxo-hexahydro-pyrrolo[3,2-b]pyrrole-1-carbonyl]-3-methyl-butyl}-4-dimethylamino-benzamide

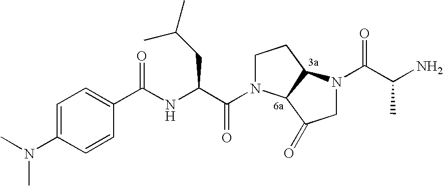

HPLC Rt=6.9-8.3 mins (>90%), HPLC-MS 458.2 [M+H]⁺, 937.4 [2M+Na]⁺.

### EXAMPLE 164

### (3aR,6aS)-4-Dimethylamino-N-{(1S)-3-methyl-1-[6-oxo-4-(thiophene-3-carbonyl)-hexahydro-pyrrolo[3,2-b]pyrrole-1-carbonyl]-butyl}-benzamide

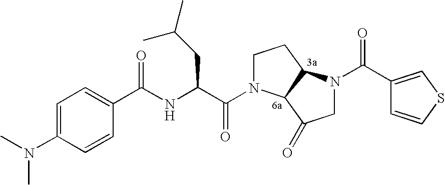

HPLC Rt=11.0-12.0 mins (>90%), HPLC-MS 497.2 [M+H]⁺, 515.2 [M+H+H₂O]⁺.

### EXAMPLE 165

### (3aR,6aS)-N-{(1S)-[4-(3-Acetylamino-thiophene-2-carbonyl)-6-oxo-hexahydro-pyrrolo[3,2-b]pyrrole-1-carbonyl]-3-methyl-butyl}-4-dimethylamino-benzamide

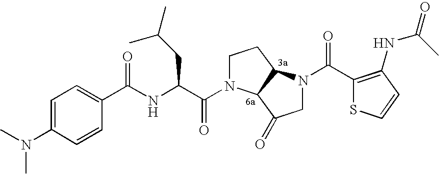

HPLC Rt=10.94 mins (>80%), HPLC-MS 554.2 [M+H]⁺, 572.2 [M+H+H₂O]⁺.

### EXAMPLE 166

### (3aR,6aS)-N-{(1S)-1-[4-(5-Chloro-thiophene-2-carbonyl)-6-oxo-hexahydro-pyrrolo[3,2-b]pyrrole-1-carbonyl]-3-methyl-butyl}-4-dimethylamino-benzamide

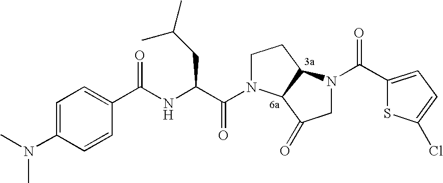

HPLC Rt=13.83 mins (>95%), HPLC-MS 531.1/533.1 [M+H]⁺, 549.1/551.1 [M+H+H₂O]⁺.

### EXAMPLE 167

### (3aS,6aR)-4-[(2S)-2-(4-Dimethylamino-benzoylamino)-4-methyl-pentanoyl]-3-oxo-hexahydro-pyrrolo[3,2-b]pyrrole-1-carboxylic acid phenylamide

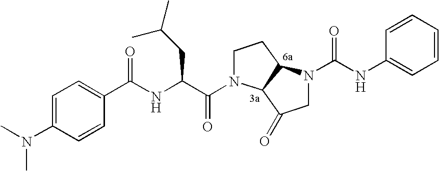

HPLC Rt=11.4-12.1 mins (>95%), HPLC-MS 506.2 [M+H]⁺.

### EXAMPLE 168

### (3aR,6aS)-4-Dimethylamino-N-{(1S)-3-methyl-1-[6-oxo-4-(2-trifluoromethoxy-benzoyl)-hexahydro-pyrrolo[3,2-b]pyrrole-1-carbonyl]-butyl}-benzamide

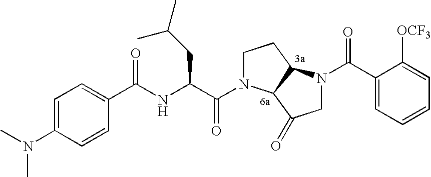

HPLC Rt=14.42 mins (>85%), HPLC-MS 575.2 [M+H]⁺, 593.2 [M+H+H₂O]⁺.

### EXAMPLE 169

### (3aR,6aS)-4-Dimethylamino-N-{(1S)-1-[4-(2-hydroxy-benzoyl)-6-oxo-hexahydro-pyrrolo[3,2-b]pyrrole-1-carbonyl]-3-methyl-butyl}-benzamide

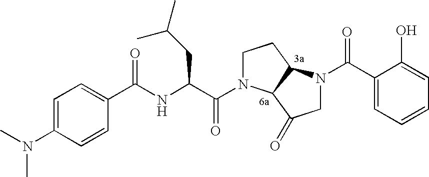

HPLC Rt=7.77 mins (>50%), HPLC-MS 507.2 [M+H]⁺.

### EXAMPLE 170

### (3aR,6aS)-N-[(1S)-1-(4-Benzoyl-6-oxo-hexahydro-pyrrolo[3,2-b]pyrrole-1-carbonyl)-3-methyl-butyl]methyl-benzamide

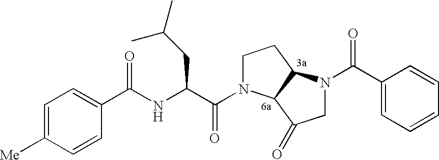

HPLC Rt=14.35 mins (>90%), HPLC-MS 462.2 [M+H]⁺, 945.3 [2M+Na]⁺.

### EXAMPLE 171

### (3aR,6aS)-N-[(1S)-1-(4-Benzoyl-6-oxo-hexahydro-pyrrolo[3,2-b]pyrrole-1-carbonyl)-3-methyl-butyl]-4-ethyl-benzamide

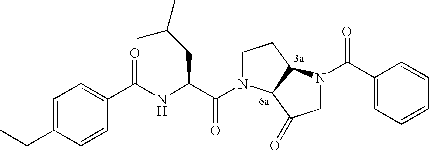

HPLC Rt=15.40 mins (>90%), HPLC-MS 476.2 [M+H]⁺, 973.4 [2M+Na]⁺.

### EXAMPLE 172

### (3aR,6aS)-N-[(1S)-1-(4-Benzoyl-6-oxo-hexahydro-pyrrolo[3,2-b]pyrrole-1-carbonyl)-3-methyl-butyl]-4-isopropyl-benzamide

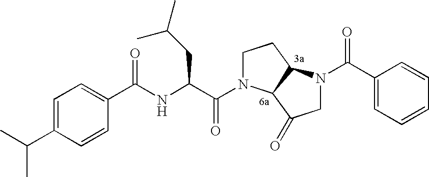

HPLC Rt=16.41 mins (>85%), HPLC-MS 490.2 [M+H]⁺.

### EXAMPLE 173

### (3aR,6aS)-N-[(1S)-1-(4-Benzoyl-6-oxo-hexahydro-pyrrolo[3,2-b]pyrrole-1-carbonyl)-3-methyl-butyl]-4-trifluoromethoxy-benzamide

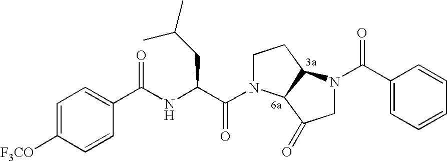

HPLC Rt=16.44 mins (>90%), HPLC-MS 532.1 [M+H]⁺.

### EXAMPLE 174

### (3aR,6aS)-N-[(1S)-1-(4-Benzoyl-6-oxo-hexahydro-pyrrolo[3,2-b]pyrrole-1-carbonyl)-3-methyl-butyl]-benzamide

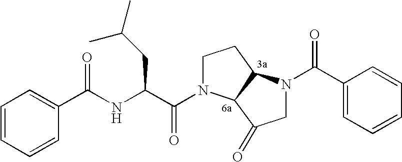

HPLC Rt=13.30 mins (>90%), HPLC-MS 448.2 [M+H]⁺, 470.1 [M+Na]⁺, 917.2 [2M+Na]⁺.

### EXAMPLE 175

### (3aS,6aR)-4-[(2S)-2-(3-Aminomethyl-benzoylamino)-4-methyl-pentanoyl]-3-oxo-hexahydro-pyrrolo[3,2-b]pyrrole-1-carboxylic acid phenylamide

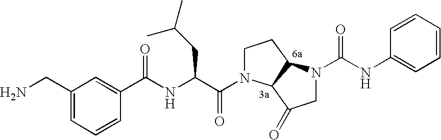

HPLC Rt=10.69 mins (>95%), HPLC-MS 492.2 [M+H]⁺, 983.4 [2M+H]⁺.

### EXAMPLE 176

### (3aR,6aS)-3-Aminomethyl-N-{(1S)-3-methyl-1-[6-oxo-4-((2R)-2-phenyl-propionyl)-hexahydro-pyrrolo[3,2-b]pyrrole-1-carbonyl]-butyl}-benzamide

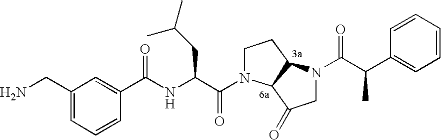

HPLC Rt=12.2-13.8 mins (>90%), HPLC-MS 505.2 [M+H]⁺, 523.2 [M+H+H₂O]⁺.

### EXAMPLE 177

### (3aR,6aS)-3-Aminomethyl-N-{(1S)-3-methyl-1-[6-oxo-4-((2S)-2-phenyl-propionyl)-hexahydro-pyrrolo[3,2-b]pyrrole-1-carbonyl]-butyl}-benzamide

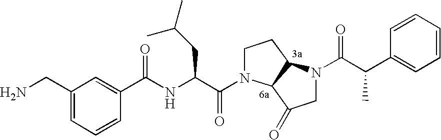

HPLC Rt=11.9-13.5 mins (>85%), HPLC-MS 505.2 [M+H]⁺.

### EXAMPLE 178

### (3aR,6aS)-3-Aminomethyl-N-((1S)-1-{4-[2-(2-fluoro-phenyl)-acetyl]-6-oxo-hexahydro-pyrrolo[3,2-b]pyrrole-1-carbonyl}-3-methyl-butyl)-benzamide

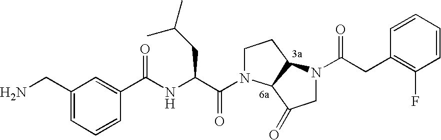

HPLC Rt=11.65 mins (>90%), HPLC-MS 509.2 [M+H]⁺.

### EXAMPLE 179

### (3aR,6aS)-3-Aminomethyl-N-((1S)-1-{4-[2-(3-fluoro-phenyl)-acetyl]-6-oxo-hexahydro-pyrrolo[3,2-b]pyrrole-1-carbonyl}-3-methyl-butyl)-benzamide

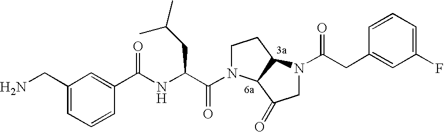

HPLC Rt=11.70 mins (>95%), HPLC-MS 509.2 [M+H]⁺.

### EXAMPLE 180

### (3aR,6aS)-3-Aminomethyl-N-((1S)-1-{4-[2-(4-fluoro-phenyl)-acetyl]-6-oxo-hexahydro-pyrrolo[3,2-b]pyrrole-1-carbonyl}-3-methyl-butyl)-benzamide

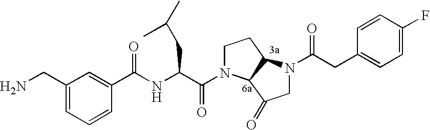

HPLC Rt=11.74 mins (>95%), HPLC-MS 509.2 [M+H]⁺.

### EXAMPLE 181

### (3aR,6aS)-3-Aminomethyl-N-{(1S)-1-[4-((2R)-2-amino-2-phenyl-acetyl)-6-oxo-hexahydro-pyrrolo[3,2-b]pyrrole-1-carbonyl]-3-methyl-butyl}-benzamide

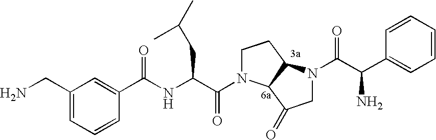

HPLC Rt=8.93 mins (>50%), HPLC-MS 506.2 [M+H]⁺, 524.2 [M+H+H₂O]⁺.

### EXAMPLE 182

### (3aR,6aS)-3-Aminomethyl-N-{(1S)-1-[4-((2S)-2-amino-2-phenyl-acetyl)-6-oxo-hexahydro-pyrrolo[3,2-b]pyrrole-1-carbonyl)-3-methyl-butyl}-benzamide

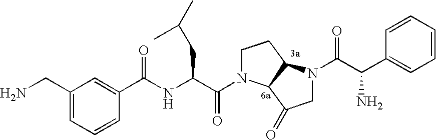

HPLC Rt=7.2-9.3 mins (>60%), HPLC-MS 506.2 [M+H]⁺, 524.2 [M+H+H₂O]⁺.

### EXAMPLE 183

### (3aS,6aR)-(2-{4-[(2S)-2-(4-Dimethylamino-benzoylamino)-4-methyl-pentanoyl]-3-oxo-hexahydro-pyrrolo[3,2-b]pyrrole-1-yl}-2-oxo-ethyl)-carbamic acid methyl ester

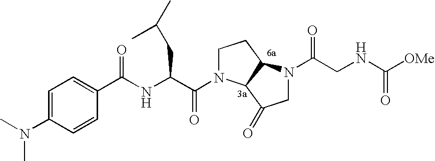

HPLC Rt=7.96 mins (>95%), HPLC-MS 502.2 [M+H]⁺.

### EXAMPLE 184

### (3aR,6aS)-4-Dimethylamino-N-((1S)-3-methyl-1-{6-oxo-4-[2-(toluene-sulfonylamino)-acetyl]-hexahydro-pyrrolo[3,2-b]pyrrole-1-carbonyl}-butyl)-benzamide

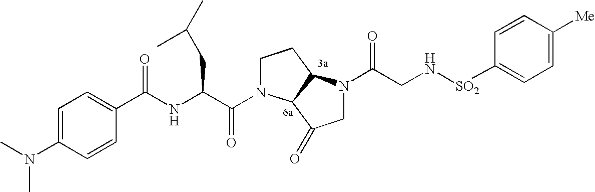

HPLC Rt=13.40 mins (>90%), HPLC-MS 598.2 [M+H]⁺.

### EXAMPLE 185

### (3aR,6aS)-3-Aminomethyl-N-{(1S)-1-[4-(2-methanesulfonyl-benzoyl)-6-oxo-hexahydro-pyrrolo[3,2-b]pyrrole-1-carbonyl]-3-methyl-butyl}-benzamide

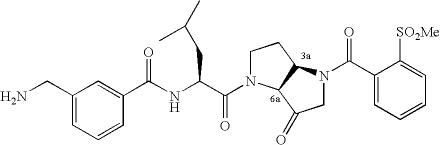

HPLC Rt=9.4-10.4 mins (>85%), HPLC-MS 555.2 [M+H]⁺, 573.2 [M+H+H₂O]⁺.

### EXAMPLE 186

### (3aR,6aS)-N-[(1S)-2-(4-Benzoyl-6-oxo-hexahydro-pyrrolo[3,2-b]pyrrol-1-yl)-1-cyclopropylmethyl-2-oxo-ethyl]-4-dimethylamino-benzamide

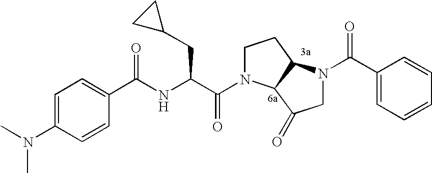

HPLC Rt=11.0-13.0 mins (>85%), HPLC-MS [489.2 M+H]⁺, 507.2 [M+H+H₂O]⁺.

### EXAMPLE 187

### (3aR,6aS)-Thiophene-3-carboxylic acid [(1S)-2-(4-benzoyl-6-oxo-hexahydro-pyrrolo[3,2-b]pyrrole-1-yl)-1-cyclopropylmethyl-2-oxo-ethyl]-amide

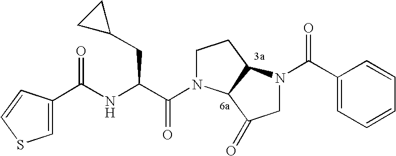

HPLC Rt=12.10 mins (>80%), HPLC-MS 452.1 [M+H]⁺, 925.2 [2M+Na]⁺.

### EXAMPLE 188

### (3aR,6aS)-1-(4-Benzoyl-6-oxo-hexahydro-pyrrolo[3,2-b]pyrrol-1-yl)-4-(3,4-dihydro-1H-isoquinolin-2-yl)-2-isobutyl-butane-1,4-dione

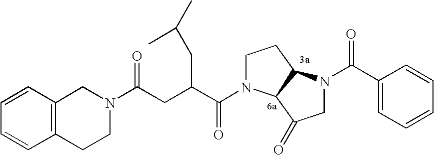

HPLC Rt=15.1-16.5 mins (>80%), HPLC-MS 502.1 [M+H]⁺, 520.2 [M+H+H₂O]⁺.

### EXAMPLE 189

### (3aS,6aR)-1-Benzoyl-4-(2-biphenyl-3-yl-4-methyl-pentanoyl)-hexahydro-pyrrolo[3,2-b]pyrrol-3-one

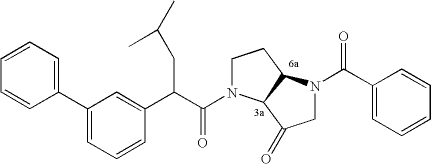

HPLC Rt=18.1-20.1 mins (>80%), HPLC-MS 481.2 [M+H]⁺, 499.2 [M+H+H₂O]⁺, 983.3 [2M+Na]⁺.

### EXAMPLE 190

### (3aS,6aR)-4-(2S-2-(4-tert-Butyl-benzoylamino)-4-methyl-pentanoyl]-3-oxo-hexahydro-pyrrolo[3,2-b]pyrrole-1-carboxylic acid phenylamide

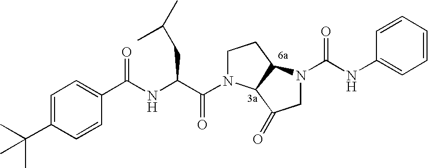

HPLC Rt=16.9-18.4 mins (>85%), HPLC-MS 519.2 [M+H]⁺.

### EXAMPLE 191

### (3aR,6aS)-4-tert-Butyl-N-{(1S)-3-methyl-1-[4-(4-methyl-pentanoyl)-6-oxo-hexahydro-pyrrolo[3,2-b]pyrrole-1-carbonyl]-butyl}-benzamide

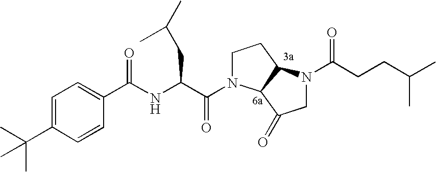

HPLC Rt=16.3-17.5 mins (>85%), HPLC-MS 498.2 [M+H]⁺.

### EXAMPLE 192

### (3aR,6aS)-N-{(1S)-1-[4-((2S)-2-Amino-4-methyl-pentanoyl)-6-oxo-hexahydro-pyrrolo[3,2-b]pyrrole-1-carbonyl]-3-methyl-butyl}-4-tert-butyl-benzamide

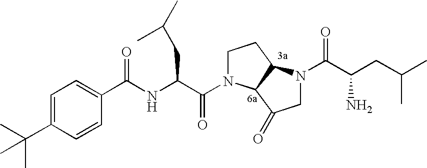

HPLC Rt=16.19 mins (>90%), HPLC-MS 513.3 [M+H]⁺.

### EXAMPLE 193

### (3aR,6aS)-N-[(1S)-1-(4-Benzoyl-6-oxo-hexahydro-pyrrolo[3,2-b]pyrrole-1-carbonyl)-3-methyl-butyl]-4-pyrrolidin-1-yl-benzamide

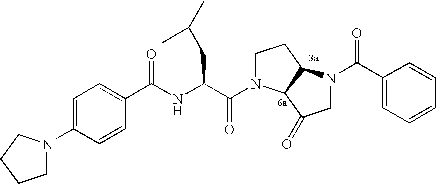

HPLC Rt=17.45 mins (>85%), HPLC-MS 517.1 [M+H]⁺, 535.2 [M+H+H₂O]⁺.

### EXAMPLE 194

### (3aR,6aS)-N-[(1S)-1-(4-Benzoyl-6-oxo-hexahydro-pyrrolo[3,2-b]pyrrole-1-carbonyl)-3-methyl-butyl]-4-piperazin-1-yl-benzamide

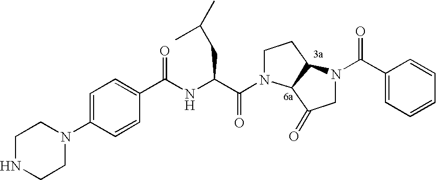

HPLC Rt=11.50 mins (>95%), HPLC-MS 532.1 [M+H]⁺, 550.2 [M+H+H₂O]⁺.

### EXAMPLE 195

### (3aR,6aS)-N-[(1S)-1-(4-Benzoyl-6-oxo-hexahydro-pyrrolo[3,2-b]pyrrole-1-carbonyl)-3-methyl-butyl]-4-dimethylamino-N-methyl-benzamide

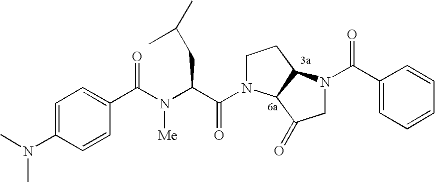

HPLC Rt=18.29 mins (>50%), HPLC-MS 505.1 [M+H]⁺.

### EXAMPLE 196

### (3aR,6aS)-N-[(1S)-1-(4-Benzoyl-6-oxo-hexahydro-pyrrolo[3,2-b]pyrrole-1-carbonyl)-butyl]-4-dimethylamino-benzamide

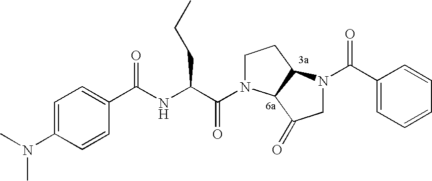

HPLC Rt=11.09 mins (>80%), HPLC-MS 477.1 [M+H]⁺, 495.1 (M+H+H₂O]⁺.

### EXAMPLE 197

### (3aR,6aS)-N-{(1S)-1-[4-((2S)-2-Acetylamino-propionyl)-6-oxo-hexahydro-pyrrolo[3,2-b]pyrrole-1-carbonyl]-3-methyl-butyl}-4-dimethylamino-benzamide

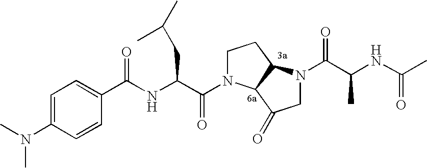

HPLC Rt=9.06 mins (>90%), HPLC-MS 500.1 [M+H]⁺, 518.2 (M+H+H₂O]⁺.

### EXAMPLE 198

### (3aR,6aS)-N-{(1S)-1-[4-(2-Amino-acetyl)-6-oxo-hexahydro-pyrrolo[3,2-b]pyrrole-1-carbonyl]-3-methyl-butyl}-4-dimethylamino-benzamide

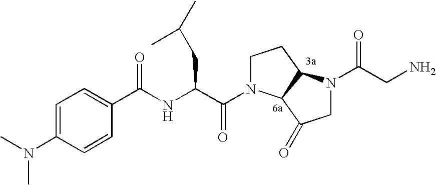

HPLC Rt=7.93 mins (>90%), HPLC-MS 444.1 [M+H]⁺.

### EXAMPLE 199

### (3aR,6aS)-N-{(1S)-1-[4-(2-Acetylamino-acetyl)-6-oxo-hexahydro-pyrrolo[3,2-b]pyrrole-1-carbonyl]-3-methyl-butyl}-4-dimethylamino-benzamide

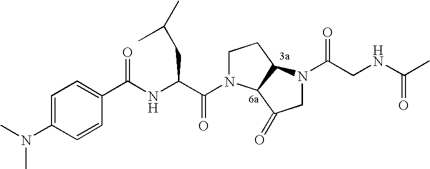

HPLC Rt=8.1 mins (>90%), HPLC-MS 486.1 [M+H]⁺.

### EXAMPLE 200

### (3aR,6aS)-4-Dimethylamino-N-{(1S)-3-methyl-1-[4-(2-methylamino-acetyl)-6-oxo-hexahydro-pyrrolo[3,2-b]pyrrole-1-carbonyl]-butyl}-benzamide

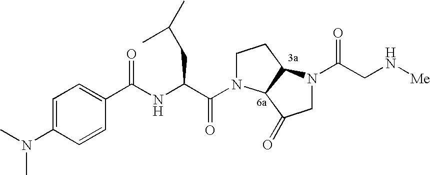

HPLC Rt=7.89 mins (>90%), HPLC-MS 458.2 [M+H]⁺.

### EXAMPLE 201

### (3aR,6aS)-N-((1S)-1-{4-[2-(Acetyl-methyl-amino)-acetyl]-6-oxo-hexahydro-pyrrolo[3,2-b]pyrrole-1-carbonyl}-3-methyl-butyl)-4-dimethylamino-benzamide

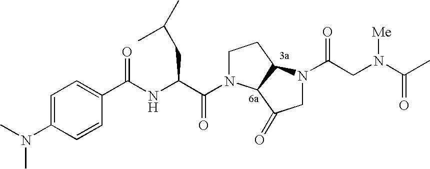

HPLC Rt=8.89 mins (>90%), HPLC-MS 500.2 [M+H]⁺.

### EXAMPLE 202

### (3aR,6aS)-N-{(1S)-1-[4-(3-Acetylamino-propionyl)-6-oxo-hexahydro-pyrrolo[3,2-b]pyrrole-1-carbonyl]-3-methyl-butyl}-4-dimethylamino-benzamide

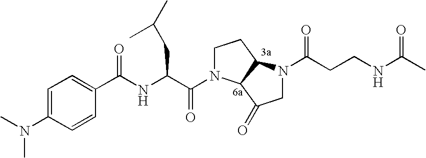

HPLC Rt=8.57 mins (>90%), HPLC-MS 500.1 [M+H]⁺.

### EXAMPLE 203

### (3aR,6aS)-N-{(1S)-1-[4-((2S)-2-Amino-butyryl)-6-oxo-hexahydro-pyrrolo[3,2-b]pyrrole-1-carbonyl]-3-methyl-butyl}-4-dimethylamino-benzamide

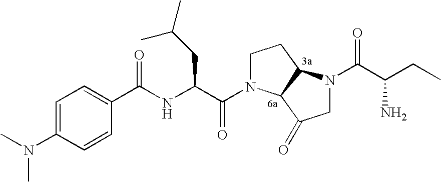

HPLC Rt=9.29 mins (>90%), HPLC-MS 472.2 [M+H]⁺.

### EXAMPLE 204

### (3aR,6aS)-N-{(1S)-1-[4-((2S)-2-Acetylamino-butyryl)-6-oxo-hexahydro-pyrrolo[3,2-b]pyrrole-1-carbonyl]-3-methyl-butyl}-4-dimethylamino-benzamide

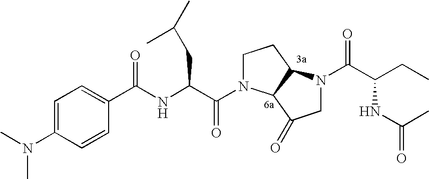

HPLC Rt=10.45 mins (>90%), HPLC-MS 514.2 [M+H]⁺.

### EXAMPLE 205

### (3aR,6aS)-N-{(1S)-1-[4-((2S)-2-Amino-3-methyl-butyryl)-6-oxo-hexahydro-pyrrolo[3,2-b]pyrrole-1-carbonyl]-3-methyl-butyl}-4-dimethylamino-benzamide

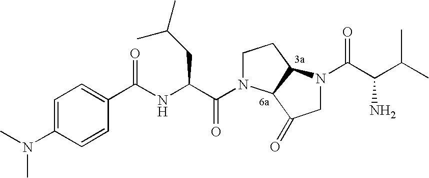

HPLC Rt=9.92 mins (>90%), HPLC-MS 486.2 [M+H]⁺.

### EXAMPLE 206

### (3aR,6aS)-N-{(1S)-1-[((2S)-2-Amino-3-hydroxy-propionyl)-6-oxo-hexahydro-pyrrolo[3,2-b]pyrrole-1-carbonyl]-3-methyl-butyl}-4-dimethylamino-benzamide

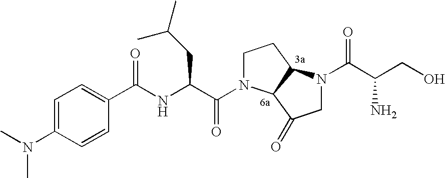

HPLC Rt=7.56 mins (>90%), HPLC-MS 474.1 [M+H]⁺.

### EXAMPLE 207

### (3aR,6aS)-N-{(1S)-1-[4-((2S,3R)-2-Amino-3-hydroxy-butyryl)-6-oxo-hexahydro-pyrrolo[3,2-b]pyrrole-1-carbonyl]-3-methyl-butyl}-4-dimethylamino-benzamide

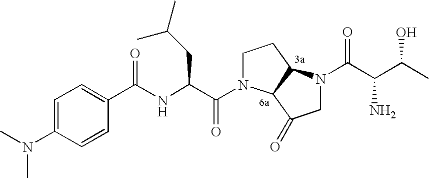

HPLC Rt=8.00 mins (>80%), HPLC-MS 488.1 [M+H]⁺.

### EXAMPLE 208

### (3aR,6aS)-N-{(1S)-1-[4-((2S,3S)-2-Amino-3-hydroxy-butyryl)-6-oxo-hexahydro-pyrrolo[3,2-b]pyrrole-1-carbonyl]-3-methyl-butyl}-4-dimethylamino-benzamide

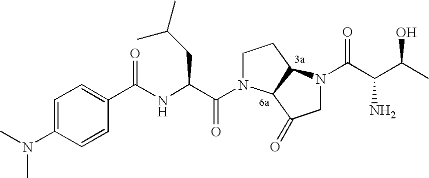

HPLC Rt=8.13 mins (>85%), HPLC-MS 488.1 [M+H]⁺.

### EXAMPLE 209

### (3aR,6aS)-N-{(1S)-1-[4-((2S,3S)-2-Amino-3-methyl-pentanoyl)-6-oxo-hexahydro-pyrrolo[3,2-b]pyrrole-1-carbonyl]-3-methyl-butyl}-4-dimethylamino-benzamide

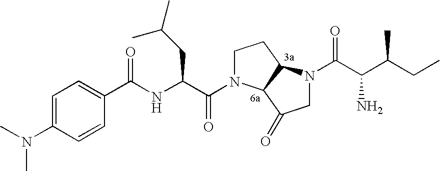

HPLC Rt=10.97 mins (>90%), HPLC-MS 500.2 [M+H]⁺.

### EXAMPLE 210

### (3aR,6aS)-N-{(1S)-1-[4-((2S)-2-Amino-pentanoyl)-6-oxo-hexahydro-pyrrolo[3,2-b]pyrrole-1-carbonyl]-3-methyl-butyl}-4-dimethylamino-benzamide

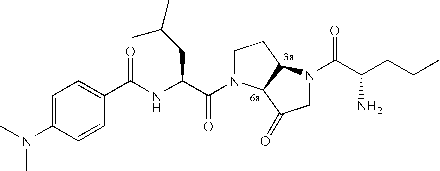

HPLC Rt=10.32 mins (>90%), HPLC-MS 486.2 [M+H]⁺.

### EXAMPLE 211

### (3aR,6aS)-N-{(1S)-1-[4-((2S)-2-Amino-3,3-dimethyl-butyryl)-6-oxo-hexahydro-pyrrolo[3,2-b]pyrrole-1-carbonyl]-3-methyl-butyl}-4-dimethylamino-benzamide

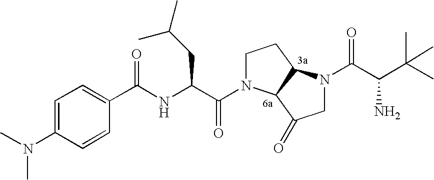

HPLC Rt=11.14 mins (>90%), HPLC-MS 500.2 [M+H]⁺.

### EXAMPLE 212

### (3aR,6aS)-N-{(1S)-1-[4-((2S)-2-Amino-4,4-dimethyl-pentanoyl)-6-oxo-hexahydro-pyrrolo[3,2-b]pyrrole-1-carbonyl]-3-methyl-butyl}-4-dimethylamino-benzamide

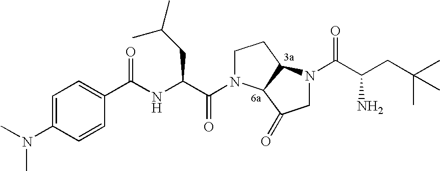

HPLC Rt=12.01 mins (>90%), HPLC-MS 514.2 [M+H]⁺.

### EXAMPLE 213

### (3aR,6aS)-N-{(1S)-1-[4-((2S)-2-Amino-5-methyl-hexanoyl)-6-oxo-hexahydro-pyrrolo[3,2-b]pyrrole-1-carbonyl]-3-methyl-butyl}-4-dimethylamino-benzamide

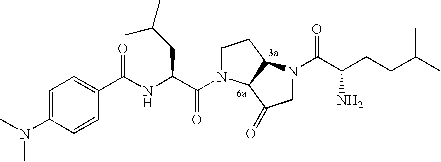

HPLC Rt=12.83 mins (>95%), HPLC-MS 514.2 [M+H]⁺.

### EXAMPLE 214

### (3aR,6aS)-N-{(1S)-1-[4-((2S)-2-Acetylamino-5-methyl-hexanoyl)-6-oxo-hexahydro-pyrrolo[3,2-b]pyrrole-1-carbonyl]-3-methyl-butyl}-4-dimethylamino-benzamide

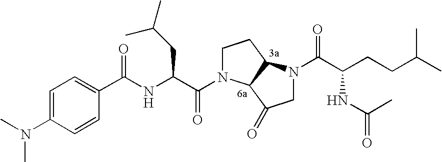

HPLC Rt=14.18 mins (>95%), HPLC-MS 556.2 [M+H]⁺.

### EXAMPLE 215

### (3aR,6aS)-N-{(1S)-1-[4-((2S)-2-Amino-3-cyclohexyl-propionyl)-6-oxo-hexahydro-pyrrolo[3,2-b]pyrrole-1-carbonyl]-3-methyl-butyl}-4-dimethylamino-benzamide

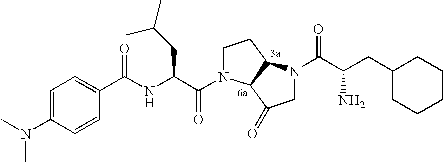

HPLC Rt=13.54 mins (>95%), HPLC-MS 540.2 [M+H]⁺.

### EXAMPLE 216

### (3aR,6aS)-N-{(1S)-1-[4-((2S)-1-Acetyl-pyrrolidine-2-carbonyl)-6-oxo-hexahydro-pyrrolo[3,2-b]pyrrole-1-carbonyl]-3-methyl-butyl}-4-dimethylamino-benzamide

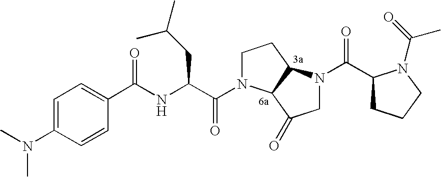

HPLC Rt=9.88 mins (>95%), HPLC-MS 526.2 [M+H]⁺.

### EXAMPLE 217

### (3aR,6aS)-4-Dimethylamino-N-{(1S)-3-methyl-1-[6-oxo-4-((2S)-piperidine-2-carbonyl)-hexahydro-pyrrolo[3,2-b]pyrrole-1-carbonyl]-butyl}-benzamide

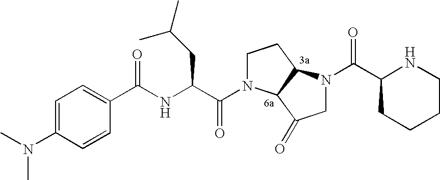

HPLC Rt=9.12 mins (>90%), HPLC-MS 498.2 [M+H]⁺, 995.3 [2M+H]⁺.

### EXAMPLE 218

### (3aR,6aS)-4-Dimethylamino-N-{(1S)-1-[4-((2S,4R)-4-hydroxy-pyrrolidine-2-carbonyl)-6-oxo-hexahydro-pyrrolo[3,2-b]pyrrole-1-carbonyl]-3-methyl-butyl}-benzamide

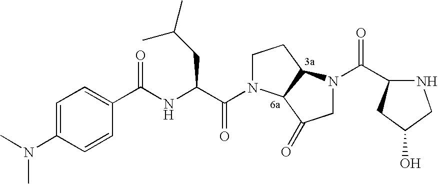

HPLC Rt=8.00 mins (>90%), HPLC-MS 500.2 [M+H]⁺, 999.3 [2M+H]⁺.

### EXAMPLE 219

### (3aR,6aS)-N-{(1S)-1-[4-((2S)-2-Amino-3-benzyloxy-propionyl)-6-oxo-hexahydro-pyrrolo[3,2-b]pyrrole-1-carbonyl]-3-methyl-butyl}-4-dimethylamino-benzamide

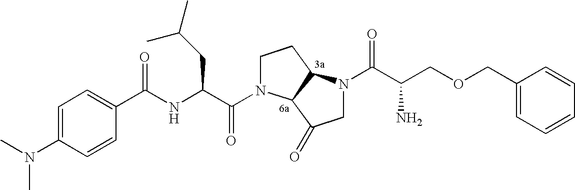

HPLC Rt=11.6-12.7 mins (>85%), HPLC-MS 564.2 [M+H]⁺.

### EXAMPLE 220

### (3aR,6aS)-4-Dimethylamino-N-{(1S)-3-methyl-1-[4-((2S)-4-methyl-2-methylamino-pentanoyl)-6-oxo-hexahydro-pyrrolo[3,2-b]pyrrole-1-carbonyl]-butyl}-benzamide

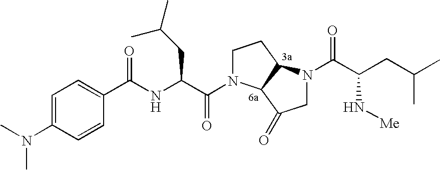

HPLC Rt=12.12 mins (>90%), HPLC-MS 514.2 [M+H]⁺.

### EXAMPLE 221

### (3aR,6aS)-N-((1S)-1-{4-[(2S)-2-(Acetyl-methyl-amino)-4-methyl-pentanoyl]-6-oxo-hexahydro-pyrrolo[3,2-b]pyrrole-1-carbonyl}-3-methyl-butyl)-4-dimethylamino-benzamide

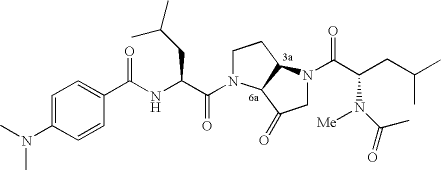

HPLC Rt=13.37 mins (>50%), HPLC-MS 556.2 [M+H]⁺.

### EXAMPLE 222

### (3aR,6aS)-N-{(1S)-1-[4-(1-Amino-cyclopropanecarbonyl)-6-oxo-hexahydro-pyrrolo[3,2-b]pyrrole-1-carbonyl]-3-methyl-butyl}-4-dimethylamino-benzamide

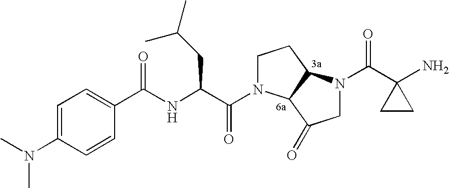

HPLC Rt=8.16 mins (>50%), HPLC-MS 470.2 [M+H]⁺.

### EXAMPLE 223

### (3aR,6aS)-N-{(1S)-1-[4-(1-Amino-cyclobutanecarbonyl)-6-oxo-hexahydro-pyrrolo[3,2-b]pyrrole-1-carbonyl]-3-methyl-butyl}-4-dimethylamino-benzamide

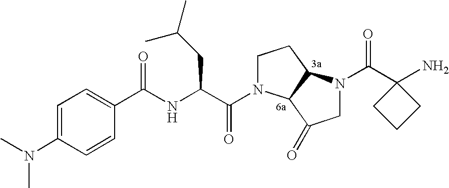

HPLC Rt=8.4-8.9 mins (>75%), HPLC-MS 484.2 [M+H]⁺.

### EXAMPLE 224

### (3aR,6aS)-N-{(1S)-1-[4-(1-Amino-cyclopentanecarbonyl-6-oxo-hexahydro-pyrrolo[3,2-b]pyrrole-1-carbonyl]-3-methyl-butyl}-4-dimethylamino-benzamide

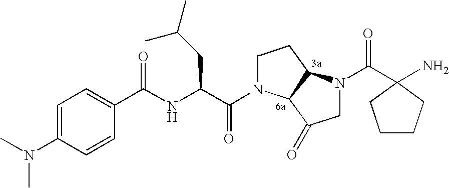

HPLC Rt=8.5-8.9 mins (>40%), HPLC-MS 498.2 [M+H]⁺.

### EXAMPLE 225

### (3aR,6aS)-N-{(1S)-1-[4-(1-Amino-cyclohexanecarbonyl)-6-oxo-hexahydro-pyrrolo[3,2-b]pyrrole-1-carbonyl]-3-methyl-butyl}-4-dimethylamino-benzamide

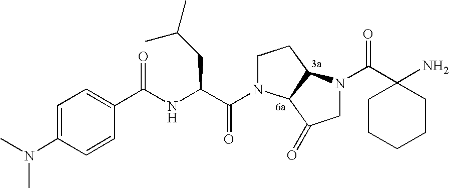

HPLC Rt=9.1-9.5 mins (>25%), HPLC-MS 529.9 [M+H+H₂O]⁺.

### EXAMPLE 226

### (3aR,6aS)-N-{(1S)-1-[4-((2S)-2-Amino-2-cyclohexyl-acetyl)-6-oxo-hexahydro-pyrrolo[3,2-b]pyrrole-1-carbonyl]-3-methyl-butyl}-4-dimethylamino-benzamide

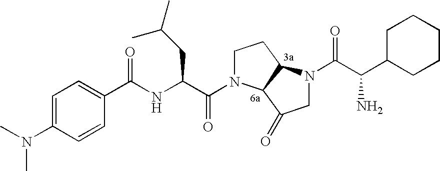

HPLC Rt=12.17 mins (>90%), HPLC-MS 526.2 [M+H]⁺.

### EXAMPLE 227

### (3aR,6aS)-4-Dimethylamino-N-{(1S)-1-[4-(2-dimethylamino-acetyl)-6-oxo-hexahydro-pyrrolo[3,2-b]pyrrole-1-carbonyl]-3-methyl-butyl}-benzamide

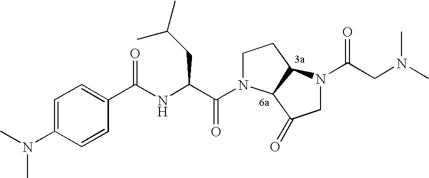

HPLC Rt=7.7-8.1 mins (>90%), HPLC-MS 472.2 [M+H]⁺, 490.2 [M+H+H₂O]⁺.

### EXAMPLE 228

### (3aR,6aS)-N-{(1S)-1-[4-((2S)-2-Amino-4-methyl-pentanoyl)-6-oxo-hexahydro-pyrrolo[3,2-b]pyrrole-1-carbonyl]-3-methyl-butyl}-4-tert-butyl-benzamide

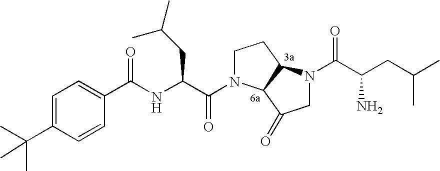

HPLC Rt=15.47 mins (>95%), HPLC-MS 513.2 [M+H]⁺.

### EXAMPLE 229

### (3aR,6aS)-4-Dimethylamino-N-{(1S)-3-methyl-1-[4-(2-methyl-butyryl)-6-oxo-hexahydro-pyrrolo[3,2-b]pyrrole-1-carbonyl]-butyl}-benzamide

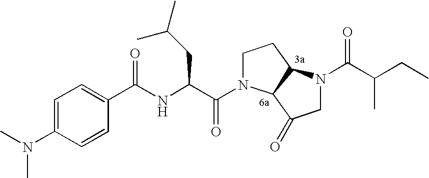

HPLC Rt=11.4-11.5 mins (>80%), HPLC-MS 471.2 [M+H]⁺, 493.2 [M+Na]⁺.

### EXAMPLE 230

### (3aR,6aS)-4-Dimethylamino-N-{(1S)-3-methyl-1-[4-(2-methyl-pentanoyl)-6-oxo-hexahydro-pyrrolo[3,2-b]pyrrole-1-carbonyl]-butyl}-benzamide

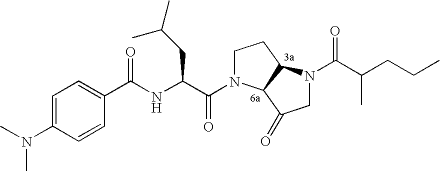

HPLC Rt=12.4-13.3 mins (>80%), HPLC-MS 485.2 [M+H]⁺.

### EXAMPLE 231

### (3aR,6aS)-4-Dimethylamino-N-{(1S)-1-[4-(2-ethyl-butyryl)-6-oxo-hexahydro-pyrrolo[3,2-b]pyrrole-1-carbonyl]-3-methyl-butyl}-benzamide

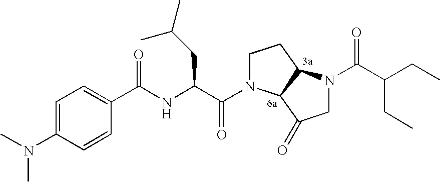

HPLC Rt=12.34 mins (>80%), HPLC-MS 485.2 [M+H]⁺, 991.3 [2M+Na]⁺.

### EXAMPLE 232

### (3aR,6aS)-4-Dimethylamino-N-{(1S)-3-methyl-1-[6-oxo-4-(2-propyl-pentanoyl)-hexahydro-pyrrolo[3,2-b]pyrrole-1-carbonyl]-butyl}-benzamide

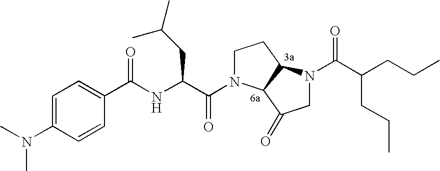

HPLC Rt=14.75 mins (>90%), HPLC-MS 513.2 [M+H]⁺.

### EXAMPLE 233

### (3aR,6aS)-4-Dimethylamino-N-{(1S)-1-[4-((2S)₂-hydroxy-4 methyl-pentanoyl)-6-oxo-hexahydro-pyrrolo[3,2-b]pyrrole-1-carbonyl]-3-methyl-butyl}-benzamide

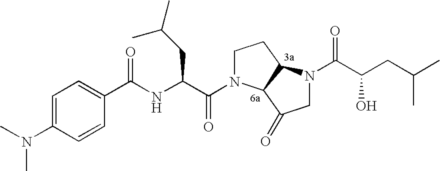

HPLC Rt=12.04 mins (>95%), HPLC-MS 501.2 [M+H]⁺, 519.2 [M+H+H₂O]⁺.

### EXAMPLE 234

### (3aR,6aS)-4-Dimethylamino-N-[(1S)-1-(4-hexanoyl-6-oxo-hexahydro-pyrrolo[3,2-b]pyrrole-1-carbonyl)-3-methyl-butyl]-benzamide

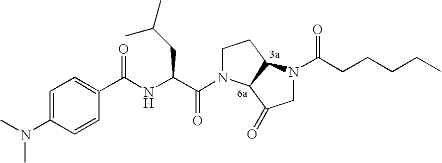

HPLC Rt=13.24 mins (>95%), HPLC-MS 485.2 [M+H]⁺, 503.2 [M+H+H₂O]⁺.

### EXAMPLE 235

### (3aR,6aS)-4-Dimethylamino-N-{(1S)-3-methyl-1-[6-oxo-4-(pyrrolidine-1-carbonyl)-hexahydro-pyrrolo[3,2-b]pyrrole-1-carbonyl]-butyl}-benzamide

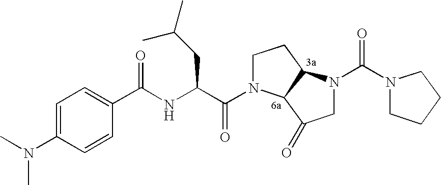

HPLC Rt=11.06 mins (>95%), HPLC-MS 484.1 [M+H]⁺, 989.3 [2M+Na]⁺.

### EXAMPLE 236

### (3aR,6aS)-4-Dimethylamino-N-{(1S)-3-methyl-1-[6-oxo-4-(piperidine-1-carbonyl)-hexahydro-pyrrolo[3,2-b]pyrrole-1-carbonyl]-butyl}-benzamide

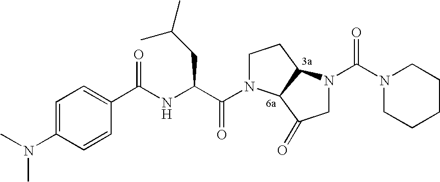

HPLC Rt=12.21 mins (>95%), HPLC-MS 498.2 [M+H]⁺, 516.3 [M+H+H₂O]⁺.

### EXAMPLE 237

### (3aR,6aS)-4-Dimethylamino-N-{(1S)-3-methyl-1-[6-oxo-4-(pyrrolidine-3-carbonyl)-hexahydro-pyrrolo[3,2-b]pyrrole-1-carbonyl]-butyl}-benzamide

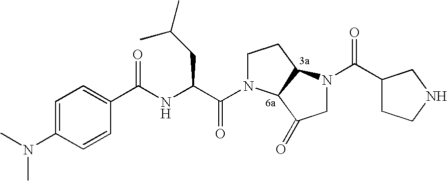

HPLC Rt=8.13 mins (>95%), HPLC-MS 484.1 [M+H]⁺, 502.2 [M+H+H₂O]⁺.

### EXAMPLE 238

### (3aR,6aS)-4-Dimethylamino-N-{(1S)-3-methyl-1-[6-oxo-4-(tetrahydro-furan-2-carbonyl)-hexahydro-pyrrolo[3,2-b]pyrrole-1-carbonyl]-butyl}-benzamide

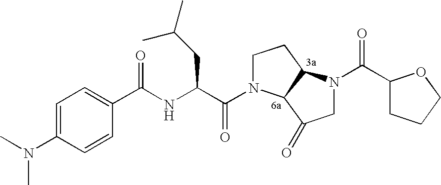

HPLC Rt=9.89 mins (>95%), HPLC-MS 485.1 [M+H]⁺, 503.1 [M+H+H₂O]⁺.

### EXAMPLE 239

### (3aR,6aS)-4-Dimethylamino-N-{(1S)-3-methyl-1-[6-oxo-4-(tetrahydro-furan-3-carbonyl)-hexahydro-pyrrolo[3,2-b]pyrrole-1-carbonyl]-butyl}-benzamide

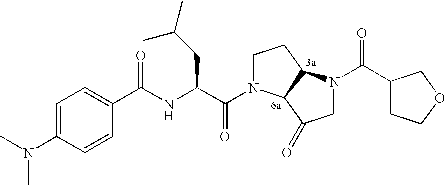

HPLC Rt=10.40 mins (>90%), HPLC-MS 485.1 [M+H]⁺, 503.1 [M+H+H₂O]⁺.

### EXAMPLE 240

### (3aR,6aS)-4-Dimethylamino-N-{(1S)-3-methyl-1-[6-oxo-4-(2-piperidinyl-acetyl)-hexahydro-pyrrolo[3,2-b]pyrrole-1-carbonyl]-butyl}-benzamide

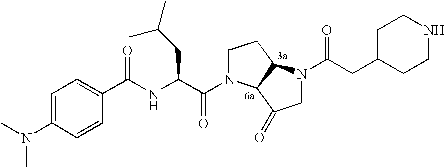

HPLC Rt=8.8-9.6 mins (>90%), HPLC-MS 512.2 [M+H]⁺, 530.2 [M+H+H₂O]⁺.

### EXAMPLE 241

### (3aR,6aS)-4-Dimethylamino-N-{(1S)-3-methyl-1-[6-oxo-4-(2-piperazin-1-yl-acetyl)-hexahydro-pyrrolo[3,2-b]pyrrole-1-carbonyl]-butyl}-benzamide

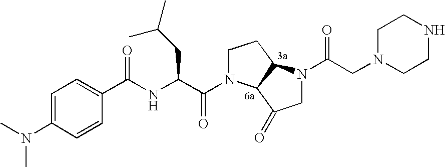

HPLC Rt=7.3-8.2 mins (>85%), HPLC-MS 513.1 [M+H]⁺, 531.2 [M+H+H₂O]⁺.

### EXAMPLE 242

### (3aR,6aS)-4-Dimethylamino-N-{(1S)-3-methyl-1-[6-oxo-4-(piperidine-4-carbonyl)-hexahydro-pyrrolo[3,2-b]pyrrole-1-carbonyl]-butyl}-benzamide

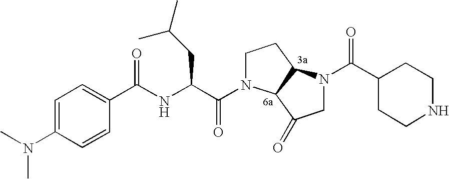

HPLC Rt=8.2-8.9 mins (>90%), HPLC-MS 498.2 [M+H]⁺, 516.2 [M+H+H₂O]⁺, 995.3 [2M+H]⁺,

### EXAMPLE 243

### (3aR,6aS)-N-{(1S)-1-[4-(Cyclopent-3-enecarbonyl)-6-oxo-hexahydro-pyrrolo[3,2-b]pyrrole-1-carbonyl]-3-methyl-butyl}-4-dimethylamino-benzamide

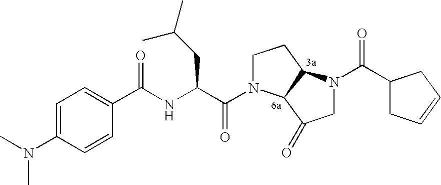

HPLC Rt=11.39 mins (>95%), HPLC-MS 481.2 [M+H]⁺, 983.3 [2M+Na]⁺.

### EXAMPLE 244

### (3aR,6aS)-N-{(1S)-1-[4-(5-Chloro-furan-2-carbonyl)-6-oxo-hexahydro-pyrrolo[3,2-b]pyrrole-1-carbonyl]-3-methyl-butyl}-4-dimethylamino-benzamide

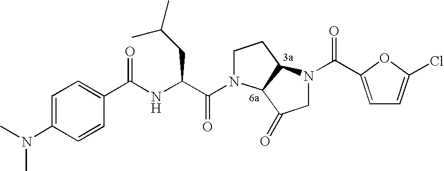

HPLC Rt=13.17 mins (90%), HPLC-MS 515.1 [M+H]⁺, 537.1 [M+Na]⁺.

### EXAMPLE 245

### (3aR,6aS)-N-{(1S)-1-[4-((2S)-2-Acetylamino-pentanoyl)-6-oxo-hexahydro-pyrrolo[3,2-b]pyrrole-1-carbonyl]-3-methyl-butyl}-4-dimethylamino-benzamide

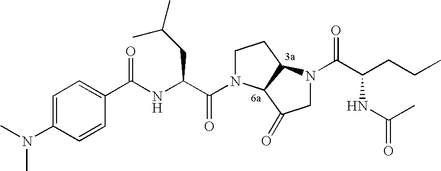

HPLC Rt=11.18 mins (>90%), HPLC-MS 528.2 [M+H]⁺.

### EXAMPLE 246

### (3aR,6aS)-3-Aminomethyl-N-{(1S)-1-[4-((2S)-2-amino-4-methyl-pentanoyl)-6-oxo-hexahydro-pyrrolo[3,2-b]pyrrole-1-carbonyl)-3-methyl-butyl}-benzamide

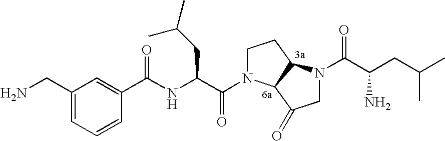

HPLC Rt=8.87 mins (>95%), HPLC-MS 486.2 [M+H]⁺, 971.4 [2M+H]⁺.

### EXAMPLE 247

### (3aR,6aS)-N-[(1S)-1-(4-Benzoyl-6-oxo-hexahydro-pyrrolo[3,2-b]pyrrole-1-carbonyl)-3-methyl-butyl]-3-piperazin-1-yl-benzamide

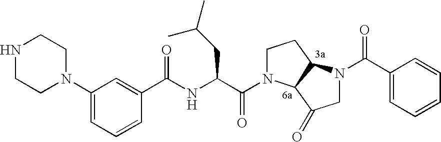

HPLC Rt=10.92 mins (>85%), HPLC-MS 532.1 [M+H]⁺, 550.2 [M+H+H₂O]⁺.

### EXAMPLE 248

### (3aS,6aS)-N-[(1S)-1-(4-Benzoyl-6-oxo-hexahydro-2-oxa-1,4-diaza-pentalene-1-carbonyl)-3-methyl-butyl]-4-dimethylamino-benzamide

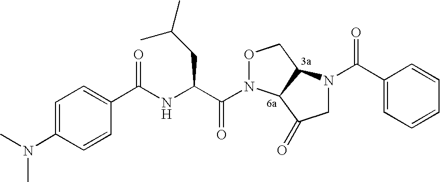

HPLC Rt=13.18 mins (>95%), HPLC-MS 493.1 [M+H]⁺, 511.1 [M+H+H₂O]⁺.

EXAMPLE 248 was prepared following the general methods detailed for EXAMPLE 1, but using an alternative building block (3aS,6aS)-6-oxo-tetrahydro-2-oxa-1,4-diazapentalene-1,4-dicarboxylic acid 4-tert-butyl ester 1-(9H-fluoren-9-ylmethyl) ester (91) prepared following general Scheme 5 and Scheme 22. Following solid phase synthesis, the crude product was purified by semi-preparative HPLC and lyophilised to give EXAMPLE 248.

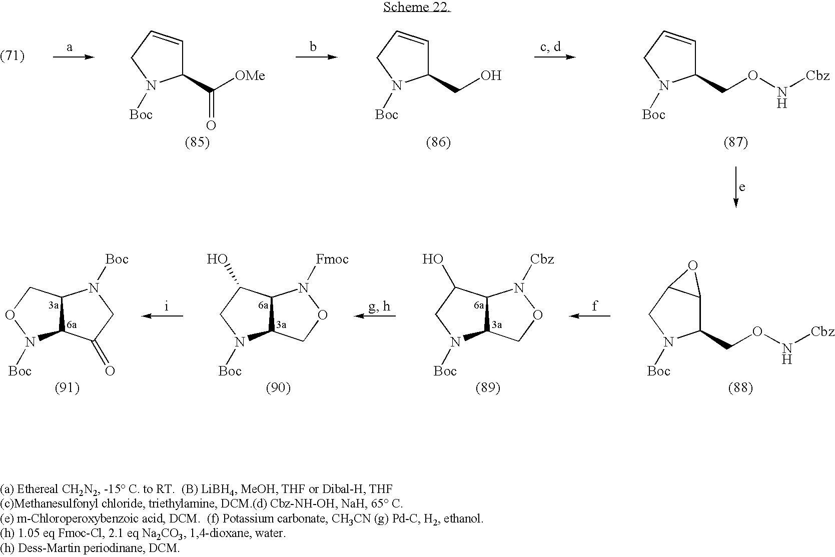

### Preparation of (S)-2,5-dihydropyrrole-1,2-dicarboxylic Acid 1-tert-butyl ester 2-methyl ester (85)

Ethereal diazomethane [˜23 mmol generated from addition of diazald (7.1 g,) in diethyl ether (115 ml) onto sodium hydroxide (8.0 g) in water (14 ml)/ethanol (28 ml) at 65° C.] was added in portions to a stirred solution of (S)-2,5-dihydropyrrole-1,2-dicarboxylic acid 1-tert-butyl ester (71) (ex Bachem, 4.98 g, 23.4 mmol) in dichloromethane (100 ml) at 0° C. over 5 minutes. The solution was stirred for 1 hour at 0° C. then glacial acetic acid (0.5 ml) was added dropwise. The product was extracted into dichloromethane (50 ml) then washed with saturated aqueous sodium hydrogen carbonate solution (100 ml), water (100 ml) and brine (100 ml). The organic layer was dried (Na₂SO₄) and the solvents removed in vacuo to obtain (S)-2,5-dihydropyrrole-1,2-dicarboxylic acid 1-tert-butyl ester 2-methyl ester (85) (4.66 g, 88%), which was used without further purification. TLC (Single spot, R_{f}=0.25, EtOAc:heptane 1:4), HPLC-MS 172.1 [M+2H−Bu]⁺, 250.1 [M+Na]⁺, 477.2 [2M+Na]⁺; C₁₁H₁₇NO₄.0.4H₂O req. (fnd.) % C 56.38 (56.47), % H 7.66 (7.25), % N 5.97 (5.88).

### Preparation of (S)-2-hydroxymethyl-2,5-dihydropyrrole-1-carboxylic acid tert-butyl ester (86)

Methanol (1.66 ml, 41 mmol) was added dropwise to a stirred suspension of lithium borohydride (0.90 g, 41 mmol) in tetrahydrofuran (20 ml) over 2 minutes under an atmosphere of argon, followed by a solution of (S)-2,5-dihydropyrrole-1,2-dicarboxylic acid 1-tert-butyl ester 2-methyl ester (85) (4.65 g, 20.5 mmol) in tetrahydrofuran (50 ml) over 15 minutes. The mixture was stirred for 70 minutes then poured into water (125 ml). The product was extracted into dichloromethane (3×100 ml), dried (Na₂SO₄), and the solvents removed in vacuo. The residue was purified by flash chromatography over silica eluting with ethyl acetate:heptane mixtures 15:85 to 25:75 to give (S)-2-hydroxymethyl-2,5-dihydropyrrole-1-carboxylic acid tert-butyl ester (86) as a colourless oil (3.75 g, 92%), [a]_{D}²² −136° (c=1, CHCl₃). TLC (Single spot, R_{f}=0.30, EtOAc:heptane 2:3), HPLC-MS 222.1 [M+Na]⁺, 421.1 [2M+Na]⁺; C₁₀H₁₇NO₃.0.3H₂O req. (fnd.) % C 58.72 (58.82), % H 8.67 (8.35), % N 6.85 (6.88); d_{H} (500 MHz, CDCl₃) mixture of rotamers (major:minor=4:1) 1.47 and 1.49 (9H total, each s, (CH₃)₃C), 3.55 (0.8H, ddd, J=11.1, 7.7, 1.1 Hz, CH₂OH major), 3.61-3.66 (0.2H, m, CH₂OH minor), 3.77 (1H, m, CH₂OH), 3.95-4.09 (1H, m, H-5), 4.14-4.19 (0.8H, m, H-5 major), 4.24-4.30 (0.2H, m, H-5 minor), 4.58 (0.2H, br. s, H-2 minor), 4.64 (1H, m, OH), 4.69-4.75 (0.8H, m, H-2 major), 5.57-5.63 and 5.78-5.82 (each 0.8H, m, H-3 and H-4 major), 5.65-5.70 and 5.89-5.94 (each 0.2H, m, H-3 and H-4 minor).

### Alternative preparation of (S)-hydroxymethyl-2,5-dihydropyrrole-1-carboxylic acid tert-butyl ester (86)

A solution of diisobutylaluminium hydride (1.0M in tetrahydrofuran, 18.7 ml, 18.7 mmol) was added dropwise to a stirred solution of (S)-2,5-dihydropyrrole-1,2-dicarboxylic acid 1-tert-butyl ester 2-methyl ester (85) (ex Bachem, 1.06 g, 4.67 mmol) in tetrahydrofuran (15 ml) at −70° C. over 45 minutes under an atmosphere of argon. The mixture was stirred for 15 minutes at −70° C. then at ambient temperature for 3.25 hours before cooling to 0° C. and adding ethyl acetate (10 ml) dropwise followed by saturated aqueous sodium potassium tartrate solution (60 ml), ethyl acetate (65 ml) and brine (60 ml). The organic layer was separated then the aqueous layer extracted with ethyl acetate (60 ml). The organic layers were combined then washed with brine (50 ml), dried (MgSO₄), and solvents removed in vacuo to leave a residue which was purified by flash chromatography over silica eluting with ethyl acetate:heptane mixtures 15:85 to 25:75 to give (S)-hydroxymethyl-2,5-dihydropyrrole-1-carboxylic acid tert-butyl ester (86) as a colourless oil (0.34 g, 36%), [a]_{D}²² −120.5° (c=1, CHCl₃). TLC (Single spot, R_{f}=0.30, EtOAc:heptane 2:3), analytical HPLC Rₜ=9.375 min; HPLC-MS 222.1 [M+Na]⁺, 421.2 [2M+Na]⁺.

### Preparation of (S)—(N′-benzyloxycarbonylaminooxymethyl)-2,5-dihydro pyrrole-1-carboxylic acid tert-butyl ester (87)

i) Pyridine (7.6 ml, 94.2 mmol) was added to a solution of (S)-hydroxymethyl-2,5-dihydropyrrole-1-carboxylic acid tert-butyl ester (86) (3.75 g, 18.8 mmol) in dichloromethane whilst stirring at 0° C. followed by methanesulfonyl chloride (1.53 ml, 19.8 mmol) in portions over 10 minutes. The mixture was stirred for 1 hour at 0° C. then at ambient temperature for 14 hours. The product was extracted into dichloromethane (250 ml), washed with ice-chilled hydrochloric acid (1M, 2×125 ml) and aqueous saturated sodium hydrogen carbonate solution (125 ml), dried (Na₂SO₄), and solvents removed in vacuo to leave (S)-methanesulfonyloxymethyl-2,5-dihydropyrrole-1-carboxylic acid tert-butyl ester as an oily residue (5.2 g, 100%) which was used without further purification. TLC (Single spot, R_{f}=0.20, EtOAc:heptane 3:7), analytical HPLC Rₜ=12.785 min; HPLC-MS 222.0 [M+2H−Bu]⁺, 577.1 [2M+Na]⁺; d_{H} (500 MHz, CDCl₃) mixture of rotamers (major:minor=4:3) 1.45 and 1.49 (9H total, each s, (CH₃)₃C), 2.96 (3H, s, SO₂CH₃), 4.02-4.03 (0.43H, m, H-5 minor), 4.04-4.07 (0.57H, m, H-5 major), 4.12-4.57 (3H, m, H-5, CH₂OS), 4.67 (0.43H, br. s, H-2), 4.74 (0.57H, br. s, H-2), 5.73-5.98 (2H, m, H-3 and H-4); d_{C} (125 MHz, CDCl₃) 28.41 (C(CH₃)₃), 36.99, 37.46 (SCH₃), 53.80, 53.95 (C-5), 62.90, 63.02 (C-2), 69.14, 69.34 (CH₂OS), 80.12, 80.62 (C(CH₃)₃), 126.08, 126.16 and 128.27, 128.36 (C-3 and C-4), 153.73, 154.13 (q, NC═O).

ii) Sodium hydride (60% dispersion in oil, 3.0 g, 75.1 mmol) was added to a stirred solution of benzyl N-hydroxycarbamate (13.2 g, 78.8 mmol) in tetrahydrofuran (200 ml) at 0° C. in portions over 30 minutes under an atmosphere of argon. The mixture was stirred for 5 minutes at 0° C. then a solution of (S)-methanesulfonyloxymethyl-2,5-dihydropyrrole-1-carboxylic acid tert-butyl ester (5.2 g, 18.6 mmol, prepared as above) in tetrahydrofuran (175 ml) was added dropwise over 15 minutes. The resulting cloudy suspension was stirred for 1 hour at ambient temperature then at 65° C. for 4 hours, followed by 14 hours at ambient temperature then 7 hours at 65° C. The product was extracted into dichloromethane (250 ml) then cautiously washed with water (250 ml). The aqueous layer was extracted with dichloromethane (250 ml) then the combined organic layers washed with water (3×150 ml) and brine (250 ml), dried (Na₂SO₄), and the solvents removed in vacuo. The residue was purified twice by flash chromatography over silica eluting with ethyl acetate:heptane mixtures 25:75 to 30:70 then 0:100 to 25:75 to give (S)—(N′-benzyloxy carbonylaminooxymethyl)-2,5-dihydropyrrole-1-carboxylic acid tert-butyl ester (87) as a colourless oil (1.67 g, 26%) together with recovered (S)-methanesulfonyloxymethyl-2,5-dihydropyrrole-1-carboxylic acid tert-butyl ester (2.44 g, 47%). Data for (S)—(N′-benzyloxycarbonylaminooxymethyl)-2,5-dihydropyrrole-1-carboxylic acid tert-butyl ester (87). TLC (Single spot, R_{f}=0.35, EtOAc:heptane 2:3), analytical HPLC Rₜ=17.141 min; HPLC-MS 349.1 [M+H]⁺, 371.1 [M+Na]⁺, 719.2 [2M+Na]⁺; Elemental analysis C₁₈H₂₄N₂O₅ req. (fnd.) % C 62.05 (62.18), % H 6.94 (7.05), % N 8.04 (7.90); HRMS C₁₈H₂₄N₂O₅Na req. 371.1583, fnd. 371.1590 (1.83 ppm); d_{H} (500 MHz, CDCl₃) mixture of rotamers (major:minor=2:1) 1.46 (9H, s, (CH₃)₃C), 3.67 (0.67H, dd, J=11.45 and 7.6 Hz, CH₂ONH major), 3.89 (0.33H, dd, J=10.2 and 6.2 Hz, CH₂ONH minor), 3.99 (0.67H, dd, J=11.45 and 3.7 Hz, CH₂ONH major), 3.95-4.10 (1H, m, H-5), 4.08-4.13 (0.33H, m, CH₂ONH minor), 4.21 (0.67H, dd, J=15.7 and 1.8 Hz, H-5 major), 4.20-4.26 (0.33H, m, H-5, minor), 4.63 (0.33H, br. s, H-2 minor), 4.85-4.90 (0.67H, m, H-2 m, major), 5.13-5.18 (2H, m, OCH₂Ph), 5.68-5.73 and 5.82-5.87 (2H, m, H-3 and H-4), 7.30-7.37 (5H, aromatics), 7.52 (0.33H, br. s, NH, minor), 8.69 (0.67H, br. s, NH, major); d_{C} (125 MHz, CDCl₃) 28.40 (C(CH₃)₃), 53.57, 53.78 (C-5), 62.18, 62.76 (C-2), 67.13, 67.55 (OCH₂Ph), 77.27, 77.86 (CH₂ONH), 80.07 (C(CH₃)₃), 126.62, 126.72, 127.47, 127.85, 128.12, 128.24, 128.50, 128.54, 128.58 (C-3, C-4, aromatic CH), 135.48, 135.87, 136.18 (aromatic quaternary), 154.04, 155.24 (CH₂NC═O), 156.94, 157.39 (ONC═O).

### Preparation of (2R)-(N′-benzyloxycarbonylaminooxymethyl)-oxa-3-aza bicyclo[3.1.0]hexane-3-carboxylic acid tert-butyl ester (88)

meta-Chloroperoxybenzoic acid (57-86%, 1.9 g, ˜7.7 mmol) was added in portions under an atmosphere of argon over 15 minutes to a stirred solution of (S)—(N′-benzyloxycarbonylaminooxymethyl)-2,5-dihydropyrrole-1-carboxylic acid tert-butyl ester (87) (600 mg, 1.72 mmol) in dichloromethane (12 ml). The mixture was stirred for 14 hours then the product was extracted into dichloromethane (50 ml), washed with aqueous saturated sodium hydrogen carbonate solution (2×30 ml), dried (Na₂SO₄), and the solvents removed in vacuo. The residue was purified by flash chromatography over silica eluting with ethyl acetate:heptane 0:100 to 30:70 to give (2R)-(N′-benzyloxycarbonylaminooxymethyl)-6-oxa-3azabicyclo[3.1.0]hexane-3-carboxylic acid tert-butyl ester (88) as a colourless oil (390 mg, 62%). TLC (Single spot, R_{f}=0.35, EtOAc:heptane 2:3), analytical HPLC Rₜ=15.733 min; HPLC-MS 265.1 [M+2H−Boc]⁺, 309.0 [M+2H−Bu]⁺, 387.1 [M+Na]⁺, 751.2 [2M+Na]⁺; C₁₈H₂₄N₂O₆.0.4H₂O req. (fnd.) % C 58.21 (58.24), % H 6.73 (6.62), % N 7.54 (7.57); HRMS C₁₈H₂₄N₂O₆Na req. 387.1532, fnd. 387.1534 (0.42 ppm).

### Preparation of (3aS,6S,6aS)-6-hydroxytetrahydro-2-oxa-1,4-diazapentalene-1,4-dicarboxylic acid 1-benzyl ester 4-tert-butyl ester (89)

Potassium carbonate (1.06 g, 7.7 mmol) was added to a stirred solution of (2R)-(N′-benzyloxycarbonylaminooxymethyl)-6-oxa-3-azabicyclo[3.1.0]hexane-3-carboxylic acid tert-butyl ester (88) (280 mg, 0.77 mmol) in acetonitrile (4 ml) under an atmosphere of argon. The suspension was stirred for 5 hours then the product was extracted into dichloromethane (30 ml) and washed with water (10 ml). The aqueous layer was extracted with dichloromethane (10 ml) then the combined organic layers washed with water (10 ml), dried (Na₂SO₄), and the solvents removed in vacuo. The residue was purified by flash chromatography over silica eluting with ethyl acetate:heptane mixtures 0:100 to 30:70 to give (3aS,6S,6aS)-6-hydroxytetrahydro-2-oxa-1,4-diazapentalene-1,4-dicarboxylic acid 1-benzyl ester 4-tert-butyl ester (89) as a colourless oil (141 mg, 50%) together with recovered (1S,2R,5R)-2-(N′-benzyloxycarbonylaminooxymethyl)-6-oxa-3-azabicyclo[3.1.0]hexane-3-carboxylic acid tert-butyl ester (88) as a colourless oil (71 mg, 25%). Data for (3aS,6S,6aS)-6-hydroxytetrahydro-2-oxa-1,4-diazapentalene-1,4-dicarboxylic acid 1-benzyl ester 4-tert-butyl ester (89). TLC (Single spot, R_{f}=0.20, EtOAc:heptane 2:3), analytical HPLC Rₜ=14.994 min; HPLC-MS 265.1 [M+2H−Boc]⁺, 309.1 [M+2H−Bu]⁺, 751.2 [2M+Na]⁺; HRMS C₁₈H₂₄N₂O₆Na req. 387.1532, fnd. 387.1529 (−0.87 ppm); d_{H} (500 MHz, CDCl₃) mixture of rotamers, tentative proton assignment, 1.45 (9H, s, C(CH₃)₃), 2.28 (1H, d, J=3.9 Hz, OH), 3.45-4.81 (7H, m, BocNCHCH₂, BocNCH, BocNCH₂, CHOH, CbzNCH), 5.12-5.26 (2H, m, OCH₂Ph), 7.32-7.42 (5H, aromatics). Data for (1S,2R,5R)-2-(N′-benzyloxycarbonylaminooxymethyl)-6-oxa-3-azabicyclo[3.1.0]hexane-3 caboxylic acid tert-butyl ester (88). TLC (Single spot, R_{f}=0.35, EtOAc:heptane 40:60), HPLC-MS 265.1 [M+2H−Boc]⁺, 309.1 [M+2H−Bu]⁺, 387.1 [M+Na]⁺, 751.2 [2M+Na]⁺; d_{H} (500 MHz, CDCl₃) mixture of rotamers, tentative proton assignment, 1.41 (9H, s, C(CH₃)₃), 3.39-3.72 (3H, m, H-2 and H-5), 3.90-4.41 (4H, m, H-3, H-4 and CH₂ON), 5.12-5.20 (2H, m, OCH₂Ph), 7.31-7.39 (5H, aromatics), 7.60 and 8.0 (0.8H total, each br. s, NH).

### Preparation of (3aS,6S,6aS)-6-hydroxytetrahydro-2-oxa-1,4-diazapentalene-1,4-dicarboxylic acid 4-tert-butyl ester 1-(9H-fluoren-9-ylmethyl) ester (90)

Ethanol (2.5 ml) was cautiously added to a stirred mixture of (3aS,6S,6aS)-6-hydroxytetrahydro-2-oxa-1,4-diazapentalene-1,4-dicarboxylic acid 1-benzyl ester 4-tert-butyl ester (89) (43 mg, 0.118 mmol) and 10% palladium on charcoal (5 mg) under an atmosphere of argon at 0° C. The argon was replaced by hydrogen then the suspension was stirred at ambient temperature for 45 minutes then the hydrogen was replaced by argon before filtering the mixture through celite in vacuo. The filter cake was washed with ethanol (25 ml) then solvents removed in vacuo from the filtrate to leave (3aS,6S,6aS)-6-hydroxyhexahydro-2-oxa-1,4-diazapentalene-4-carboxylic acid tert-butyl ester as an oily residue (28 mg), which was used without further purification. HPLC-MS 175.1 [M+2H−Bu]⁺, 483.2 [2M+Na]⁺. A solution of sodium carbonate (31 mg, 0.295 mmol) in water (1.75 ml) was added whilst stirring to a solution of (3aS,6S,6aS)-6-hydroxyhexahydro-2-oxa-1,4-diazapentalene-4-carboxylic acid tert-butyl ester (28 mg) in 1,4-dioxane (1.0 ml). The mixture was cooled to 0° C. then a solution of Fmoc-Cl (34 mg, 0.132 mmol) in 1,4-dioxane (0.75 ml) was added dropwise over 40 minutes. The mixture was stirred at 0° C. for 2.25 hours then at ambient temperature for 30 minutes. Water (20 ml) was added then the product extracted into dichloromethane (3×15 ml), dried (Na₂SO₄), and the solvents removed in vacuo. The residue was purified by flash chromatography over silica eluting with ethyl acetate:heptane mixtures 0:100 to 30:70 to give (3aS,6S,6aS)-6-hydroxytetrahydro-2-oxa-1,4-diazapentalene-1,4-dicarboxylic acid 4-tert-butyl ester 1-(9H-fluoren-9-ylmethyl) ester (90) as a white solid (40 mg, 75%). TLC (Single spot, R_{f}=0.20, EtOAc:heptane 3:7), analytical HPLC Rt=18.217 min; HPLC-MS 475.1 [+Na]⁺, 927.2 [2M+Na]⁺; C₂₅H₂₈N₂O₆.0.5EtOAc req. (fnd.) % C 65.35 (64.85), % H 6.50 (6.21), % N 5.64 (5.66).

### Preparation of (3aS,6aS)-6-oxo-tetrahydro-2-oxa-1,4-diazapentalene-1,4-dicarboxylic acid 4-tert-butyl ester 1-(9H-fluoren-9-ylmethyl) ester (91)

Dess-Martin periodinane (73 mg, 0.170 mmol) was added to a stirred solution of (3aS,6S,6aS)-6-hydroxytetrahydro-2-oxa-1,4-diazapentalene-1,4-dicaboxylic acid 4-tert-butyl ester 1-(9H-fluoren-9-ylmethyl) ester (90) (39 mg, 0.086 mmol) in dichloromethane (1.25 ml). The mixture was stirred for 2.5 hours, stored at −80° C. for 14 hours, and then purified by flash chromatography over silica eluting with ethyl acetate:heptane mixtures 5:95 to 15:85 to give (3aS,6aS)-6-oxo-tetrahydro-2-oxa-1,4-diazapentalene-1,4-dicarboxylic acid 4-tert-butyl ester 1-(9H-fluoren-9-ylmethyl) ester (91) as a white solid (31 mg, 80%). TLC (Single spot, R_{f}=0.30, EtOAc:heptane 2:3), analytical HPLC broad peak Rₜ=19.57-22.15 min; HPLC-MS single broad main UV peak, 473.1 [M+Na]⁺, 491.1 [M+H₂O+Na]⁺, 923.1 [2M+Na]⁺, 959.1 [2M+2H₂O+Na]⁺; HRMS C₂₅H₂₆N₂O₆Na req. 473.1689, fnd. 473.1690 (0.24 ppm); d_{H} (500 MHz, CDCl₃) mixture of rotamers major:minor 1.5:1, 1.48 (5.4H, s, C(CH₃)₃ major), 1.50 (3.6H, s, C(CH₃)₃ minor), 3.49-3.58 (1H, m, BocNCHCH₂), 3.78-3.92 (2H, m, BocNCH₂), 4.13 (0.4H, d, J=9.5 Hz, BocNCHCH₂ [minor]), 4.20-4.29 (1.6H, m, Fmoc-CH and BocNCHCH₂ [major]), 4.46-4.52 (1H, m, Fmoc-CH₂), 4.60-4.74 (2.4H, m, Fmoc-CH₂, FmocNCH, BocNCH [minor]), 4.83 (0.6H, dd, J=7.5 and 4.3 Hz, BocNCH [major]), 7.29-7.78 (8H, aromatic); d_{C} (125 MHz, CDCl₃) 28.38, 28.31 (C(CH₃)₃), 46.96, 47.05 (Fmoc-CH), 52.40, 52.93 (BocNCH₂), 61.95 (BocNCH), 64.48, 65.31 (FmocNCH), 68.59, 68.76 (Fmoc-CH₂), 77.17, 77.31 (BocNCHCH₂), 81.61 (C(CH₃)₃), 120.02, 125.11, 125.35, 127.21, 127.28, 127.98 (Fmoc aromatic CH), 141.29, 141.33, 143.04, 143.12 (Fmoc quaternary), 153.09, 154.00 (Boc C═O), 157.64 (Fmoc C═O), 204.85, 205.44 (C═O).

Broadly following the general details from Scheme 6, the required bicycle building block (3aS,6aS)-6-oxo-tetrahydro-2-oxa-1,4-diazapentalene-1,4-dicarboxylic acid 4-tert-butyl ester 1-(9H-fluoren-9-ylmethyl) ester (91) was converted to the corresponding equivalent of building block-linker construct (27) (where Pg₂ is tert-butoxycarbonyl) as follows:
A solution of sodium acetate trihydrate (24 mg, 0.173 mmol) in water (0.25 ml) was added to a solution of (3aS,6aS)-6-oxo-tetrahydro-2-oxa-1,4-diazapentalene-1,4-dicarboxylic acid 4-tert-butyl ester 1-(9H-fluoren-9-ylmethyl) ester (91) (26 mg, 0.058 mmol) and 4-[[(hydrazinocarbonyl)amino]methyl]cyclohexane carboxylic acid. trifluoroacetate (Murphy, A. M., et al, J. Am. Chem. Soc, 114, 3156-3157, 1992) (38 mg, 0.116 mmol) in ethanol (1.75 ml). The reaction heated at 75° C. in a sealed tube for 1.5 hour. The product was extracted into chloroform (50 ml) then washed with hydrochloric acid (0.1M, 2×25 ml), saturated aqueous sodium chloride solution (30 ml) then dried (Na₂SO₄) and the solvent removed in vacuo to leave the product as a white solid (37 mg, ˜100%). Analytical HPLC has main UV peaks with Rt=20.223 and 21.596 mins and HPLC-MS (main UV peaks each with 648.2 [M+H]⁺).

Following the general details from Scheme 6, the corresponding building block-linker construct was attached to the solid phase providing loaded building block-linker construct following standard loading protocols and indicated quantitative loading.

EXAMPLES 249a to 249c were prepared entirely by solution phase synthesis methods (broadly defined by the general strategy detailed in Scheme 4) following Schemes 23 to 25 and have utility as inhibitors of cathepsin K with Ki<1000 nM.

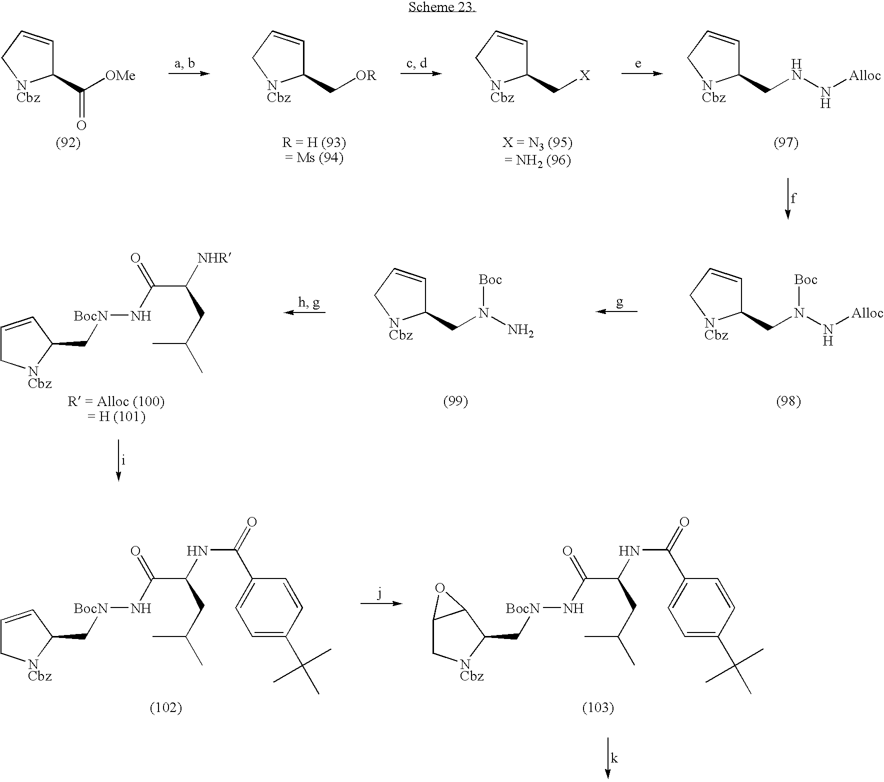

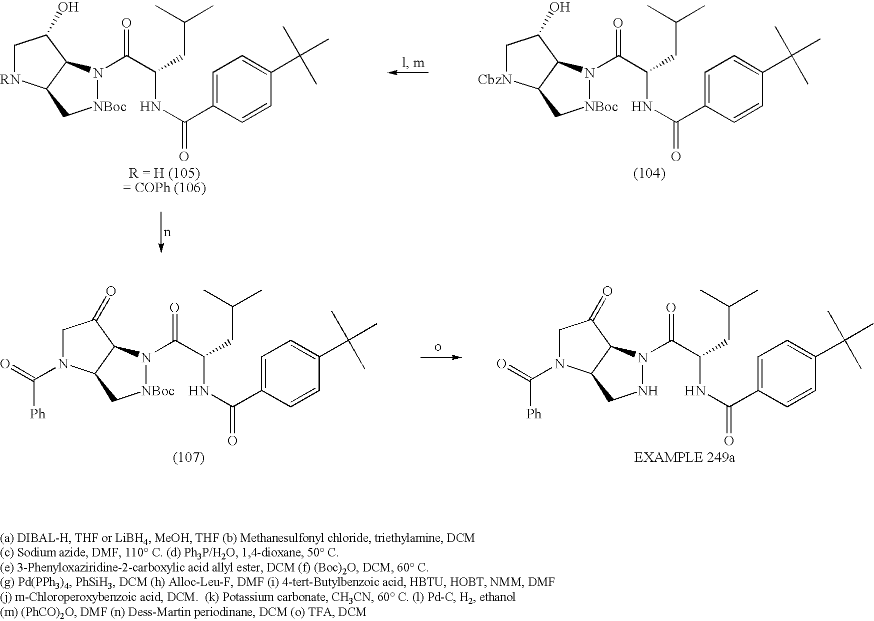

### Preparation of (S)-2,5-dihydropyrrole-1,2-dicarboxylic acid 1-benzyl ester 2-methyl ester (92)

Carbonic acid benzyl ester 2,5-dioxopyrrolidin-1-yl ester (8.45 g, 33.9 mmol) then triethylamine (10.8 ml, 77 mmol) were added dropwise to a stirred solution of (S)-2,5-dihydropyrrole-1,2-dicarboxylic acid 1-benzyl ester 2-methyl ester hydrochloride (5.0 g, 30.6 mmol) and THF:water (1:1, 306 ml) at 0° C. The mixture was stirred at ambient temperature for 12 hours then half of the solvent was removed in vacuo. The product was extracted into tert-butyl methyl ether (3×100 ml) then the combined organic layers were washed with 5% hydrochloric acid (100 ml), 5% aqueous sodium hydrogen carbonate solution (100 ml) and brine (100 ml), dried (MgSO₄), and the solvents removed in vacuo. The residue was purified by flash chromatography over silica eluting with heptane:tert-butyl methyl ether 2:1 to give (S)-2,5-dihydro pyrrole-1,2-dicarboxylic acid 1-benzyl ester 2-methyl ester (92) as a pale yellow oil (7.9 g, 99%). TLC (R_{f}=0.30, heptane:tert-butyl methyl ether 1:1), analytical HPLC single peak with Rₜ=13.935 min, HPLC-MS 262.0 [M+H]⁺, 284.0 [M+Na]⁺, 545.1 [2M+Na]⁺. δ_{H} (500 MHz, CDCl₃) approximately 1:1 mixture of rotamers, 3.57 and 3.64 (3H, each s, OCH₃), 4.22-4.36 (2H, m, H-5), 5.03-5.14 (3H, m, OCH₂Ph and H-2), 5.69-5.78 and 5.92-5.99 (2H, each m, H-3 and H-4), 7.29-7.39 (5H, aromatics); δ_{C} (125 MHz, CDCl₃) δ2.25 and 52.42 (OCH₃), 53.36 and 53.85 (C-5), 66.25 and 66.56 (C-2), 67.09 and 67.16 (PhCH₂O), 124.66, 127.80, 127.91, 127.98, 128.03, 128.40, 128.46, 129.09 and 129.18 (C-3, C-4 and aromatic CH), 136.43 and 136.51 (aromatic quaternary), 153.91 and 154.36 (NC═O), 170.38 and 170.62 (CHC═O).

### Preparation of (S)-2-hydroxymethyl-2,5-dihydropyrrole-1-carboxylic acid benzyl ester (93)

Diisobutylaluminium hydride (1.5M in toluene, 2.62 ml, 3.93 mmol) was added dropwise over 20 minutes to a stirred solution of (S)-2,5-dihydropyrrole-1,2-dicarboxylic acid 1-benzyl ester 2-methyl ester (92) (0.41 g, 1.57 mmol) in THF (15 ml), at −78° C. under an atmosphere of argon. The mixture was stirred for 2 hours at −78° C. then at ambient temperature for 18 hours. Saturated aqueous potassium sodium tartrate solution (40 ml) was added slowly to the mixture, followed by ethyl acetate (40 ml) and magnesium sulphate ˜5 g. The resultant slurry was vigorously stirred for 2 hours, then filtered and the filter cake washed with ethyl acetate. The filtrate was concentrated in vacuo. The residue was purified by flash chromatography over silica eluting with ethyl acetate:heptane 1:4 to give (S)-2-hydroxymethyl-2,5-dihydropyrrole-1-carboxylic acid benzyl ester (93) as a pale yellow oil (130 mg, 36%). TLC (R_{f}=0.30, heptane:ethyl acetate 1:1), analytical HPLC single peak with Rₜ=11.033 min, HPLC-MS 234.1 [M+H]⁺, 256.0 [M+Na]⁺, 489.1 [2M+Na]⁺. d_{H} (500 MHz, CDCl₃) approximately 4:1 mixture of rotamers, 3.58-3.66 (1H, m, CH₂OH), 3.77-3.85 (1H, m, CH₂OH), 4.14-4.32 (3H, m, CH₂OH and H-5), 4.63-4.68 (0.2H, br. s, H-2 minor), 4.76-4.81 (0.8H, m, H-2 major), 5.14-5.21 (2H, m, OCH₂Ph), 5.63-5.66 and 5.81-5.85 (1.6H, m, H-3 and H-4 major), 5.69-5.73 and 5.90-5.96 (0.4H, m, H-3 and H-4 minor), 7.29-7.39 (5H, aromatics); d_{C} (125 MHz, CDCl₃) δ3.98 (major) and 54.59 (minor) (C-5), 64.27 (minor) and 66.65 (major) (CH₂OH), 66.11 (minor) and 68.08 (major) (C-2), 67.41 (PhCH₂O), 126.67, 126.70, 126.96, 127.19, 127.40, 127.62, 127.95, 128.17, 128.54 and 128.60 (C-3, C-4 and aromatic CH), 136.30 (aromatic quaternary), 156.68 (NC═O).

### Alternative preparation of (S)-2-hydroxymethyl-2,5-dihydropyrrole-1-carboxylic acid benzyl ester (93)

Methanol (2.43 ml) followed by a solution of (S)-2,5-dihydropyrrole-1,2-dicarboxylic acid 1-benzyl ester 2-methyl ester (92) (7.90 g, 30.2 mmol) in THF (125 ml) were added dropwise to a stirred suspension of lithium borohydride (1.32 g, 60.5 mmol) in THF (45 ml). The mixture was stirred for 1 hour then water (10 ml) cautiously added dropwise. The product was extracted into tert-butyl methyl ether (3×100 ml) then the combined organic layers dried (MgSO₄), and the solvents removed in vacuo. The residue was purified by flash chromatography over silica eluting with heptane:ethyl acetate 4:1 to give (S)-2-hydroxymethyl-2,5-dihydropyrrole-1-carboxylic acid benzyl ester (93) as a pale yellow oil (6.38 g, 90%). TLC (R_{f}=0.30, heptane:ethyl acetate 1:1), analytical HPLC single peak with Rₜ=11.036 min, HPLC-MS 234.1 [M+H]⁺, 256.0 [M+Na]⁺, 489.1 [2M+Na]⁺.

### Preparation of (S)-2-methanesulfonyloxymethyl-2,5-dihydropyrrole-1-carboxylic acid benzyl ester (94)

Triethylamine (337 μl, 2.4 mmol) was added dropwise to a stirred solution of (S)-2-hydroxymethyl-2,5-dihydropyrrole-1-carboxylic acid benzyl ester (93) (0.35 g, 1.50 mmol) and methanesulfonyl chloride (174 μl, 2.25 mmol) in dichloromethane (10 ml) at 0° C. The mixture was stirred for 30 minutes then washed with water (10 ml) and brine (10 ml), dried (Na₂SO₄), and the solvents removed in vacuo to give (S)-2-methanesulfonyloxymethyl-2,5-dihydropyrrole-1-carboxylic acid benzyl ester (94) as a pale yellow oil (443 mg, 95%) which was used without further purification. TLC (R_{f}=0.30, heptane:ethyl acetate 1:1), analytical HPLC single peak with Rₜ=14.115 min, HPLC-MS 312.0 [M+H]⁺, 334.0 [M+Na]⁺, 645.1 [2M+Na]⁺;

### Preparation of (S)-2-azidomethyl-2,5-dihydropyrrole-1-carboxylic acid benzyl ester (95)

Sodium azide (8.89 g, 137 mmol) was added to a stirred solution of (S)-2-methanesulfonyloxymethyl-2,5-dihydropyrrole-1-carboxylic acid benzyl ester (94) (8.52 g, 27.4 mmol) in DMF (150 ml). The reaction mixture was stirred at 110° C. for 1 hour. The solvent was removed in vacuo then the product extracted into ethyl acetate (300 ml), washed with water (300 ml), brine (200 ml), dried (MgSO₄), and the solvents removed in vacuo. The residue was purified by flash chromatography over silica eluting with heptane:ethyl acetate 9:1 to give (S)-2-azidomethyl-2,5-dihydropyrrole-1-carboxylic acid benzyl ester (95) as a pale yellow oil (5.05 g, 72%). TLC (R_{f}=0.65, heptane:ethyl acetate 1:1), analytical HPLC single peak with Rₜ=17.855 min, HPLC-MS 259.0 [M+H]⁺, 281.0 [M+Na]⁺, 539.1 [2M+Na]⁺.

### Preparation of (S)-2-aminomethyl-2,5-dihydropyrrole-1-carboxylic acid benzyl ester (96)

Triphenylphosphine (3.20 g, 12.2 mmol) was added to a stirred solution of (S)-2-azidomethyl-2,5-dihydropyrrole-1-carboxylic acid benzyl ester (95) (2.10 g, 8.13 mmol) in THF (170 ml) containing water (2 ml). The mixture was stirred at 50° C. for 2.5 hours then at ambient temperature 16 hours. The were solvents removed in vacuo then the residue was purified by flash chromatography over silica eluting with dichloromethane:methanol 99:1 to 95:5 mixtures to give (S)-2-aminomethyl-2,5-dihydropyrrole-1-carboxylic acid benzyl ester (96) as a pale yellow oil (2.15 g) which was contaminated with triphenylphosphine oxide. The pale yellow oil was dissolved in tert-butyl methyl ether (15 ml) then cooled to 0° C. before adding HCl in 1,4-dioxane (4M, 5 ml) followed by iced-water (20 ml). The aqueous layer was extracted with tert-butyl methyl ether (3×20 ml), then the pH adjusted to ˜12 using 1M aqueous sodium hydroxide solution. The product was then extracted into dichloromethane (3×50 ml) and the combined dichloromethane layers were dried (MgSO₄), and the solvents removed in vacuo to give (S)-2-aminomethyl-2,5-dihydropyrrole-1-carboxylic acid benzyl ester (96) as a pale yellow oil (1.40 g, 74%). TLC (R_{f}=0.20, methanol:dichloromethane 1:9), HPLC-MS 233.1 [M+H]⁺, 255.1 [M+Na]⁺, 487.1 [2M+Na]⁺. d_{H} (500 MHz, D₆-DMSO) 1.30-2.10 (1H, br. s, CH₂NH₂), 3.00-3.70 (1H, br. s, CH₂NH₂), 2.64-2.75 (1H, m, CH₂NH₂), 2.79-2.88 (1H, m, CH₂NH₂), 4.00-4.21 (2H, m, H-5), 4.42-4.47 (1H, m, H-2), 5.05-5.16 (2H, m, PhCH₂O), 5.84-6.01 (2H, m, H-4 and H-5), 7.31-7.40 (5H, aromatics); d_{C} (125 MHz, D₆-DMSO) 43.91 and 44.85 (CH₂NH₂), 53.86 and 54.47 (C-5), 65.81 and 65.99 (PhCH₂O), 66.41 and 67.17 (C-2), 126.30, 126.36, 127.55, 127.58, 127.84, 127.86, 128.48, 128.51, 128.83 and 129.06 (C-3, C-4 and aromatic CH), 137.13 and 137.17 (aromatic quaternary), 153.82 and 153.97 (NC═O).

### Preparation of (S)-2-(N′-allyloxycarbonylhydrazinomethyl)-2,5-dihydro pyrrole-1-carboxylic acid benzyl ester (97)

i) Preparation of 3-phenyloxaziridine-2-carboxylic Acid Allyl Ester

Ice-chilled sodium hydroxide (2M, 50 ml, 100 mmol) was added to a vigorously stirred solution of benzaldehyde (5.3 g, 50 mmol) in diethyl ether (50 ml) at ambient temperature, then ice-chilled solutions of hydroxylamine-O-sulfuric acid (6.0 g, 53 mmol) in water (50 ml) and sodium hydroxide (2M, 25 ml, 50 mmol) were added simultaneously over 20 minutes. Allyl chloroformate (5.31 ml, 50 mmol) was added dropwise over 5 minutes then the mixture was stirred at 0° C. for 10 minutes before separating the ethereal layer. The aqueous phase was extracted with diethyl ether (2×25 ml), then the combined organic layers stirred at 0° C. for 10 minutes with a solution of hydroxylamine-O-sulfuric acid (2.5 g, 21 mmol) in water (25 ml). The phases were separated, then the aqueous phase extracted with diethyl ether (2×25 ml). The combined ethereal layers were dried (MgSO₄), and the solvents removed in vacuo. The brown oily residue was purified by flash chromatography over silica eluting with heptane:ethyl acetate 9:1 to give 3-phenyloxaziridine-2-carboxylic acid allyl ester as a pale yellow oil (1.44 g, 14%). TLC (R_{f}=0.7, heptane:ethyl acetate 1:1), HPLC-MS 206.0 [M+H]⁺, 228.1 [M+Na]⁺, 433.0 [2M+Na]⁺.

ii) 3-Phenyloxaziridine-2-carboxylic acid allyl ester (prepared as above, 1.16 g, 5.63 mmol) was added to a stirred solution of (S)-2-aminomethyl-2,5-dihydropyrrole-1-carboxylic acid benzyl ester (96) (0.35 g, 1.50 mmol) in dichloromethane (10 ml). The mixture was stirred for 16 hours then the solvents were removed in vacuo. The residue was purified by flash chromatography over silica eluting with ethyl acetate:heptane mixtures 1:9 to 1:4 to give (S)-2-(N′-allyloxycarbonylhydrazinomethyl)-2,5-dihydropyrrole-1-carboxylic acid benzyl ester (97) as a pale yellow oil (0.18 g, 36%). TLC (R_{f}=0.45, heptane:ethyl acetate 1:1), analytical HPLC Rt=14.789 min, HPLC-MS 332.1 [M+H]⁺, 354.1 [M+Na]⁺, 685.2 [2M+Na]⁺. d_{H} (500 MHz, CDCl₃) mixture of rotamers, tentative assignment of spectrum 2.73-3.40 (2H, m, CH₂NHNH), 4.08-4.80 (5H, m, H-2, H-5 and CH₂CH═CH₂), 4.95-5.35 (4H, m, PhCH₂O and CH₂CH═CH₂), 5.57-5.98 (3H, m, H-4, H-5 and CH₂CH═CH₂), 7.25-7.55 (5H, aromatics); d_{C} (125 Mz, CDCl₃) δ3.61, 53.65, 54.02 and 54.07 (C-5 and CH₂NHNH), 65.24 (C-2), 65.87, 66.21, 66.43, 66.82 and 67.13 (CH₂CH═CH₂ and PhCH₂O), 117.48, 117.83, 118.20 and 118.49 (CH₂CH═CH₂), 127.00, 127.13, 127.70, 127.79, 127.93, 128.02, 128.13, 128.33, 128.52 and 128.54 (C-3, C-4 and aromatic CH), 132.11, 132.17, 132.21, 132.46, 132.54 and 132.68 (CH₂CH═CH₂), 136.65 (aromatic quaternary), 154.52, 154.82, 155.85 and 156.21 (NNHC═O), 158.69 (CH₂NC═O).

### Preparation of (S)-2-(N-tert-butoxycarbonyl-N′-allyloxycarbonylhydrazino methyl)-2,5-dihydropyrrole-1-carboxylic acid benzyl ester (98)

Three portions of Boc anhydride (each 1.20 g, 5.55 mmol) were added at one hour intervals to a stirred solution of (S)-2-(N′-allyloxycarbonylhydrazinomethyl)-2,5-dihydropyrrole-1-carboxylic acid benzyl ester (97) (184 mg, 0.56 mmol) in triethylamine:methanol (1:9, 10 ml). The mixture was stirred at 60° C. for 3 hours then the solvents were removed in vacuo. The residue was purified by flash chromatography over silica eluting with ethyl acetate:heptane mixtures 1:9 to 1:4 to give (S)-2-(N-tert-butoxycarbonyl-N′-allyloxycarbonylhydrazinomethyl)-2,5-dihydropyrrole-1-carboxylic acid benzyl ester (98) as a pale yellow oil (190 mg, 79%). TLC (R_{f}=0.5, heptane:ethyl acetate 1:1), analytical HPLC Rₜ=19.828 min, HPLC-MS 432.2 [M+H]⁺, 454.1 [M+Na]⁺, 885.2 [2M+Na]⁺. d_{H} (500 MHz, CDCl₃) mixture of rotamers approximately 4:1, 1.38-1.45 (9H, br. s, C(CH₃)₃), 3.45-3.74 (2H, m, CH₂NNH), 4.02-4.16 (1H, m, H-5), 4.24-4.38 (1H, m, H-5), 4.54-4.68 (2H, m, CH₂CH═CH₂), 4.72 (minor), 4.84 (major) (1H total, each br. s, H-2), 5.03-5.20 (2H, m, PhCH₂O), 5.18-5.34 (2H, m, CH₂CH═CH₂), 5.76 (1H, br. s, H-3), 5.79-5.85 (1H, m, H-4), 5.84-5.95 (1H, m, CH₂CH═CH₂), 7.28-7.40 (5H, aromatics); d_{C} (125 MHz, CDCl₃) 28.07 (C(CH₃)₃), 53.32 (CH₂NNH), 53.86 (C-5), 62.45 and 62.86 (C-2), 66.21 and 67.02 (CH₂CH═CH₂ and PhCH₂O), 81.03 and 81.43 (C(CH₃)₃), 117.97 and 118.26 (CH₂CH═CH₂), 126.63, 128.00, 128.47 and 128.68 (C-3, C-4 and aromatic CH), 132.22 and 132.42 (CH₂CH═CH₂), 136.48 (aromatic quaternary), 154.96 and 155.57 (NHNHC═O and CH₂NC═O).

### Preparation of (S)-2-(N-tert-butoxycarbonylhydrazinomethyl)-2,5-dihydro pyrrole-1-carboxylic acid benzyl ester (99)

Tetrakis(triphenylphosphine)palladium(0) (10.2 mg, 0.0088 mmol) was added to a stirred solution of (S)-2-(N-tert-butoxycarbonyl-N′-allyloxycarbonylhydrazine methyl)-2,5-dihydropyrrole-1-carboxylic acid benzyl ester (98) (190 mg, 0.44 mmol) in dichloromethane (10 ml) under an atmosphere of argon. Phenylsilane (0.109 ml, 0.88 mmol) was then added dropwise over two minutes. The solution was stirred for 1 hour then the solvents were removed in vacuo. The oily residue was purified by flash chromatography over silica eluting with heptane:tert-butyl methyl ether 9:1 to 0:1 mixtures to give (S)-2-(N-tert-butoxycarbonylhydrazinomethyl)-2,5-dihydropyrrole-1-carboxylic acid benzyl ester (99) as a pale yellow oil (148 mg, 97%). TLC (R_{f}=0.35, heptane:ethyl acetate 1:1), analytical HPLC Rₜ=15.669 min, HPLC-MS 248.1 [M+2H−Boc]⁺, 292.1 [M+2H−Bu]⁺, 370.1 [M+Na]⁺, 717.3 [2M+Na]⁺. d_{H} (500 MHz, CDCl₃) 1.41-1.45 (9H, br. s, C(CH₃)₃), 1.45-1.70 (2H, br. s, NH₂), 3.53-3.95 (2H, m, CH₂NNH₂), 4.03-4.12 (1H, m, H-5), 4.25-4.36 (1H, r, H-5), 4.74-4.91 (1H, m, H-2), 5.04-5.26 (2H, m, PhCH₂O), 5.73-5.87 (2H, m, H-4 and H-5), 7.28-7.41 (5H, aromatics); d_{C} (125 MHz, CDCl₃) 28.26 (C(CH₃)₃), 53.23 (C-5), 53.70 (CH₂NNH₂), 63.24 (C-2), 66.73 and 67.18 (PhCH₂O), 80.25 and 80.53 (C(CH₃)₃), 126.01, 127.82, 127.91, 128.08, 128.22, 128.42 and 128.49 (C-3 C-4, and aromatic CH), 136.69 (aromatic quaternary).

### Preparation of (2S)-2-[N′-((2S)-2-allyloxycarbonylamino-4-methylpentanoyl)-N-tert-butoxycarbonylhydrazinomethyl]-2,5-dihydropyrrole-1-carboxylic acid benzyl ester (100)

(i) Preparation of Alloc-L-Leucine Fluoride (Alloc-Leu-F)

Alloc-L-Leucine (0.90 g, 4.2 mmol) was dissolved in dichloromethane (50 ml) with stirring under nitrogen. (Diethylamino)sulfur trifluoride (DAST, 790 μl, 6.0 mmol) was added and the mixture stirred for 1.75 hours. The mixture was added to iced-water (200 ml) and product extracted into dichloromethane (50 ml), dried (MgSO₄), and reduced in vacuo to a mobile tan oil (0.70 g, 77%). An analytical sample, pre-treated with 10% pyridine in methanol gave HPLC-MS 230.1 [M+H]⁺, 481.1 [2M+Na]⁺ (methyl ester).

(ii) Alloc-Leu-F (prepared as above, 47 mg, 0.21 mmol) was dissolved in dimethylformamide (1.5 ml) then added to (S)-2-(N-tert-butoxycarbonyl-1-hydrazinomethyl)-2,5-dihydropyrrole-1-carboxylic acid benzyl ester (99) (71 mg, 0.20 mmol) under an atmosphere of nitrogen. The solution was stirred for 19 hours then the solvents were removed in vacuo. The residue was purified by flash chromatography over silica eluting with ethyl acetate:heptane mixtures 1:9 to 3:7 to give (2S)-2-[N′-((2S)-2-allyloxycarbonylamino-4-methylpentanoyl)-N-tert-butoxycarbonylhydrazinomethyl]-2,5-dihydropyrrole-1-carboxylic acid benzyl ester (100) as a sticky white solid (69 mg, 63%). TLC (Single spot, R_{f}=0.75, EtOAc:heptane 2:1), analytical HPLC broad double peak Rₜ=21.214 and 21.483 min; HPLC-MS 445.2 [M+2H−Boc]⁺, 489.2 [M+2H−Bu]⁺, 545.2 [M+H]⁺.

### Preparation of (2S)-2-[N′-((2S)-2-amino-4-methylpentanoyl)-N-tert-butoxy carbonylhydrazinomethyl]-2,5-dihydropyrrole-1-carboxylic acid benzyl ester (101)

Dichloromethane (1.5 ml) followed by phenylsilane (32 μl, 0.26 mmol) were consecutively added with stirring under an atmosphere of nitrogen to a mixture of tetrakistriphenylphosphine palladium(0) (3.0 mg, 0.003 mmol) and (2S)-[N′-((2S)-2-allyloxycarbonylamino-4-methylpentanoyl)-N-tert-butoxycarbonyl hydrazinomethyl]-2,5-dihydropyrrole-1-carboxylic acid benzyl ester (100) (70 mg, 0.129 mmol). The solution was stirred for 80 minutes then purified by flash chromatography over silica eluting with methanol:dichloromethane mixtures 0:100 to 5:95 to give (2S)-2-[N′-((2S)-2-amino-4-methylpentanoyl)-N-tert-butoxycarbonylhydrazinomethyl]-2,5-dihydropyrrole-1-carboxylic acid benzyl ester (101) as a colourless oil (57 mg, 96%). TLC (Single spot, R_{f}=0.65, MeOH:dichloromethane 1:9), analytical HPLC Rₜ=16.345 min; HPLC-MS 461.2 [M+H]⁺, 483.2 [M+Na]⁺, 921.4 [2M+H]⁺.

### Preparation of (2S)-2-{N-tert-butoxycarbonyl-N′-[(2S)-2-(4-tert-butylbenzoyl amino)-4-methylpentanoyl]-hydrazinoylmethyl}-2,5-dihydropyrrole-1-carboxylic acid benzyl ester (102)

4-Methylmorpholine (26.6 μl, 0.244 mmol) was added to a solution of HBTU (46 mg, 0.122 mmol), 1-hydroxybenzotriazole monohydrate (18.6 mg, 0.122 mmol) and 4-(tert-butyl)benzoic acid (22 mg, 0.122 mmol) in dimethylformamide (1.5 ml). The solution was stood for 5 minutes then added to (2S)-2-[N′-((2S)-2-amino-4-methylpentanoyl)-N-tert-butoxycarbonylhydrazinomethyl]-2,5-dihydropyrrole-1-carboxylic acid benzyl ester (101) (56 mg, 0.122 mmol). The mixture was stirred for 1 hour then the solvents were removed in vacuo (water bath temperature<33° C.). The residue was dissolved in dichloromethane (15 ml) then washed with pH 3 hydrochloric acid (5 ml), saturated aqueous sodium hydrogen carbonate solution (5 ml) and brine (5 ml), dried (Na₂SO₄), and the solvents removed in vacuo. The yellow residue (108 mg) was purified by flash chromatography over silica eluting with ethyl acetate:heptane mixtures 1:9 to 3:7 to give (2S)-2-{N-tert-butoxycarbonyl-N′-[(2S)-2-(4-tert-butylbenzoylamino)-4-methylpentanoyl]-hydrazinoylmethyl}-2,5-dihydropyrrole-1-carboxylic acid benzyl ester (102) as a sticky white solid (63 mg, 84%). TLC (R_{f}=0.55, EtOAc:heptane 1:1), analytical HPLC Rₜ=24.205 min; HPLC-MS 521.2 [M+2H−Boc]⁺, 621.3 [M+H]⁺.

### Preparation of (2S)-2-{N-tert-butoxycarbonyl-N′-[(2S)-2-(4-tert-butylbenzoyl amino)-4-methylpentanoyl]-hydrazinomethyl}-6-oxa-3-aza-bicyclo[3.1.0]hexane-3-carboxylic acid benzyl ester (103)

A solution of meta-chloroperoxybenzoic acid (57-86%, 196 mg, ˜0.81 mmol) in dichloromethane (1.2 ml) was added to (2S)-2-{N-tert-butoxycarbonyl-N′-[(2S)-2-(4-tert-butylbenzoylamino)-4-methylpentanoyl)-hydrazinoylmethyl}-2,5-dihydropyrrole-1-carboxylic acid benzyl ester (102) (50 mg, 0.081 mmol) under an atmosphere of nitrogen. The solution was stirred for 20 hours then dichloromethane (15 ml) was added and the mixture washed with 5% aqueous sodium hydroxide solution (10 ml), then 10% aqueous sodium hydroxide solution (5 ml), dried (Na₂SO₄), and the solvents removed in vacuo. The residue was purified by flash chromatography over silica eluting with ethyl acetate:heptane mixtures 0:100 to 40:60 to give (2S)-2-{N-tert-butoxycarbonyl-N′-[(2S)-2-(4-tert-butylbenzoylamino)-4-methylpentanoyl]-hydrazino methyl}-6-oxa-3-aza-bicyclo[3.1.0]hexane-3-carboxylic acid benzyl ester (103) as a white solid (39 mg, 75%). TLC (R_{f}=0.30, EtOAc:heptane 2:3), analytical HPLC Rₜ=23.156 min; HPLC-MS 537.2 [M+2H−Boc]⁺, 637.2 [M+H]⁺.

### Preparation of (3aR,6S,6aS)-1-[(2S)-2-(4-tert-butylbenzoylamino)-4-methylpentanoyl]-hydroxyhexahydropyrrolo[3,2-c]pyrazole-2,4-dicarboxylic acid 4-benzyl ester 2-tert-butyl ester (104)

A solution of (2S)-2-{N-tert-butoxycarbonyl-N′-[(2S)-2-(4-tert-butyl benzoylamino)-4-methylpentanoyl]-hydrazinomethyl}-6-oxa-3-aza-bicyclo[3.1.0] hexane-3-carboxylic acid benzyl ester (103) (38.5 mg, 0.061 mmol) in acetonitrile (4.0 ml) was added to potassium carbonate (210 mg, 1.51 mmol). The suspension was placed under an atmosphere of nitrogen then heated at 60° C. whilst stirring for 4.75 hours before being allowed to cool to ambient temperature. The suspension was filtered then the filtrate concentrated in vacuo. The residue was purified by flash chromatography over silica eluting with ethyl acetate:heptane mixtures 0:100 to 50:50 to give (3aR,6S,6aS)-1-[(2S)-2-(4-tert-butylbenzoylamino)methylpentanoyl]-6-hydroxyhexahydropyrrolo[3,2-c]pyrazole-2,4-dicarboxylic acid 4-benzyl ester 2-tert-butyl ester (104) as a white solid (16.2 mg, 42%). TLC (R_{f}=0.30, EtOAc:heptane 1:1), analytical HPLC Rₜ=21.762 min; HPLC-MS 537.2 [M+2H−Boc]⁺, 581.1 [M+2H−Bu]⁺, 637.2 [M+H]⁺.

### Preparation of (3aR,6S,6aS)-4-benzoyl-1-[(2S)-2-(4-tert-butylbenzoylamino)-4-methylpentanoyl]-6-hydroxyhexahydropyrrolo[3,2-c]pyrazole-2-carboxylic acid tert-butyl ester (106)

Under an atmosphere of nitrogen a solution of (3aR,6S,6aS)-1-[(2S)-2-(4-tert-butylbenzoylamino)-4-methylpentanoyl]-6-hydroxyhexahydropyrrolo[3,2-c]pyrazole-2,4-dicarboxylic acid 4-benzyl ester 2-tert-butyl ester (104) (16.0 mg, 0.025 mmol) in ethanol (1.5 ml) was added to 10% palladium on charcoal (10 mg) whilst stirring. The nitrogen was replaced by hydrogen then stirring continued for 30 minutes. The hydrogen was replaced by nitrogen then the mixture filtered through celite. The filter cake was washed with ethanol (40 ml) then the filtrate concentrated in vacuo. The residue was used without further purification. Analytical HPLC Rₜ=18.568 min; HPLC-MS 403.2 [M+2H−Boc]⁺, 503.2 [M+H]⁺ for (3aR,6S,6aS)-1-[(2S)-2-(4-tert-butylbenzoylamino)-4-methylpentanoyl]-6-hydroxyhexahydropyrrolo[3,2-c] pyrazole-2-carboxylic acid tert-butyl ester (105).

Benzoic anhydride (6.0 mg, 0.026 mmol), dimethylformamide (0.3 ml) then 4-methylmorpholine (5.8 μl, 0.053 mmol) were added consecutively to (3aR,6S,6aS)-1-[(2S)-2-(4-tert-butylbenzoylamino)-4-methylpentanoyl]-6-hydroxyhexa hydropyrrolo[3,2-c]pyrazole-2-carboxylic acid tert-butyl ester (105) (−0.025 mmol, prepared as above). The solution was stirred for 65 minutes then the majority of solvents were removed in vacuo. The residue was dissolved in ethyl acetate (10 ml), then washed with saturated aqueous sodium hydrogen carbonate solution (5 ml), pH 3 hydrochloric acid (5 ml) and brine (5 ml), dried (Na₂SO₄), and the solvents removed in vacuo. The residue (15.5 mg) was purified by flash chromatography over silica eluting with ethyl acetate:heptane mixtures 5:95 to 50:50 to give (3aR,6S,6aS)-4-benzoyl-1-[(2S)-2-(4-tert-butylbenzoylamino) methylpentanoyl]-6-hydroxyhexa hydropyrrolo[3,2-c]pyrazole-2-carboxylic acid tert-butyl ester (106) as a white solid (10.3 mg, 68%). TLC (R_{f}=0.25, EtOAc:heptane 1:1), analytical HPLC Rₜ=22.101 min; HPLC-MS 278.1, 507.2 [M+2H−Boc]⁺, 607.2 [M+H]⁺.

### Preparation of (3aR,6aS)-4-benzoyl-1-[(2S)-2-(4-tert-butylbenzoylamino)-4-methylpentanoyl]-6-oxo-hexahydropyrrolo[3,2-c]pyrazole-2-carboxylic acid tert-butyl ester (107)

A solution of Dess-Martin periodinane (54 mg, 0.128 mmol) in dichloromethane (1.25 ml) was added to (3aR,6S,6aS)-4-benzoyl-1-[(2S)-2-(4-tert-butylbenzoylamino)-4-methylpentanoyl]-6-hydroxyhexahydropyrrolo[3,2-c]pyrazole-2-carboxylic acid tert-butyl ester (106) (15.5 mg, 0.026 mmol) under an atmosphere of nitrogen. The mixture was stirred for 4.5 hours then purified by flash chromatography over silica eluting with ethyl acetate:heptane mixtures 1:9 to 2:3 to give (3aR,6aS)-4-benzoyl-1-[(2S)-2-(4-tert-butylbenzoylamino)₄ methylpentanoyl]-6-oxo-hexahydro pyrrolo[3,2-c]pyrazole-2-carboxylic acid tert-butyl ester (107) as a white solid (12.8 mg, 81%). TLC (R_{f}=0.65, EtOAc:heptane 3:2), analytical HPLC broad peak Rₜ=21.02-23.60 min; HPLC-MS single broad main UV peak 274.1, 505.2 [M+2H−Boc]⁺, 549.1 [M+2H−Bu]⁺, 605.2 [M+H]⁺, 623.2 [M+H₂O+H]⁺, 627.2 [M+Na]⁺, 645.2 [M+H₂O+Na]⁺.

### Preparation of (3aR,6aS)-N-[(1S)-1-(4-benzoyl-6-oxo-hexahydropyrrolo[3,2-c] pyrazole-1-carbonyl)-3-methylbutyl]-4-tert-butylbenzamide

### EXAMPLE 249a

Trifluoroacetic acid (0.15 ml) was added to (3aR,6aS)-4-benzoyl-1-[(2S)-2-(4-tert-butylbenzoylamino)-4-methylpentanoyl]-6-oxo-hexahydropyrrolo[3,2-c] pyrazole-2-carboxylic acid tert-butyl ester (107) (10.3 mg, 0.017 mmol) under an atmosphere of nitrogen. The solution was stirred for 45 minutes then cautiously added to saturated aqueous sodium hydrogen carbonate solution (10 ml). The product was extracted into dichloromethane (10 ml) then washed with water (10 ml), dried (Na₂SO₄), and the solvents removed in vacuo. The residue (10 mg) was combined with a second batch of material (0.8 mg, prepared in a similar way to above from 1.14 mg of (3aR,6aS)-4-benzoyl-1-[(2S)-2-(4-tert-butyl benzoylamino)-4-methylpentanoyl]-6-oxo-hexahydropyrrolo[3,2-c]pyrazole-2-carboxylic acid tert-butyl ester (107), then purified by flash chromatography over silica eluting with ethyl acetate:heptane mixtures 20:80 to 65:35 to give (3aR,6aS)-N-[(1S)-1-(4-benzoyl-6-oxo-hexahydropyrrolo[3,2-c]pyrazole-1-carbonyl)-3-methylbutyl]-4-tert-butylbenzamide (EXAMPLE 249a) as an off-white solid (3.49 mg, 37%), together with recovered (3aR,6aS)-4-benzoyl-1-[(2S)-2-(4-tert-butylbenzoylamino)-4-methylpentanoyl]-6-oxo-hexahydropyrrolo[3,2-c]pyrazole-2-carboxylic acid tert-butyl ester (107) (1.83 mg, 16%). Data for (3aR,6aS)-N-[(1S)-1-(4-benzoyl-6-oxo-hexahydropyrrolo[3,2-c]pyrazole-1 carbonyl)-3-methyl butyl]tert-butylbenzamide (EXAMPLE 249a), TLC (R_{f}=0.26, EtOAc:heptane 3:1), analytical HPLC broad peak Rₜ=18.10-19.70 min; HPLC-MS single broad main UV peak 274.1, 505.1 [M+H]⁺, 523.2 [M+H₂O+H]⁺.

An alternative preparation of (3aR,6S,6aS)-4-benzoyl-1-[(2S)-2-(4-tert-butylbenzoylamino)-4-methylpentanoyl]-6-hydroxyhexahydropyrrolo[3,2-c]pyrazole-2-carboxylic acid tert-butyl ester (106) is detailed in Scheme 24.

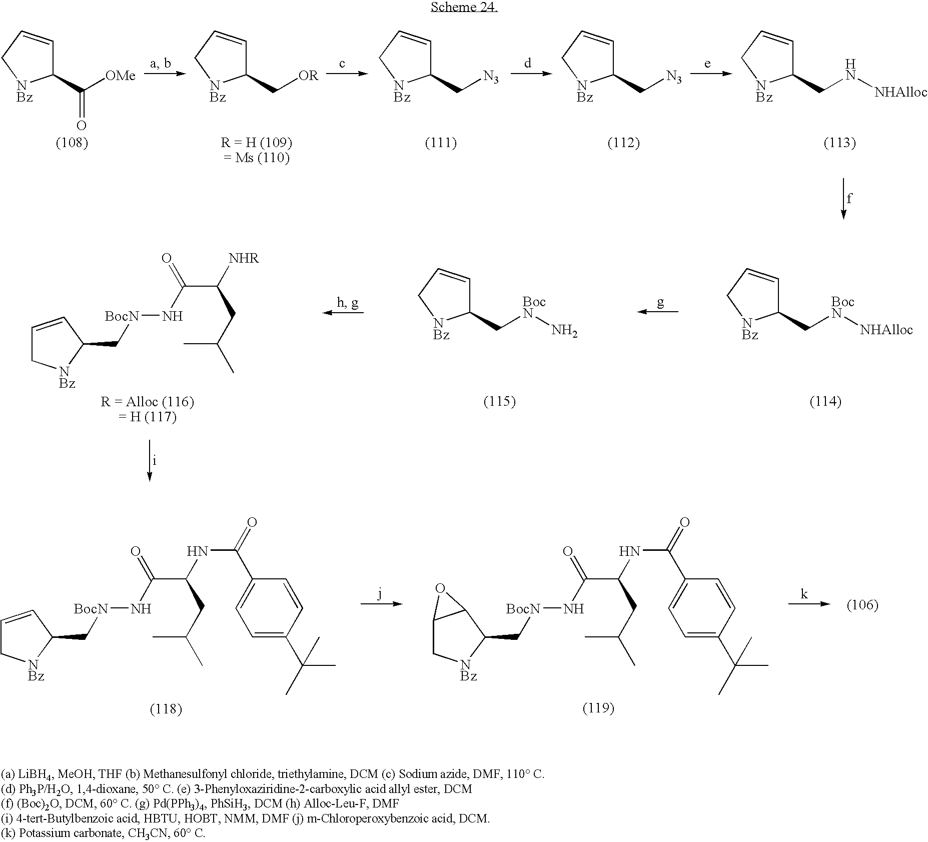

### Preparation of (S)-1-benzoyl-2,5-dihydro-1H-pyrrole-2-carboxylic acid 1-methyl ester (108)

Benzoic anhydride (4.15 g, 18.3 mmol) followed by 4-methylmorpholine (2.82 ml, 25.7 mmol) were consecutively added to a stirred solution of (S)-2,5-dihydro-1H-pyrrole-2-carboxylic acid methyl ester hydrochloride (2.0 g, 12.2 mmol) in DMF (50 ml). The mixture was stirred for 1.5 hours then the solvents were removed in vacuo. The product was extracted into tert-butyl methyl ether (300 ml) then washed with 5% hydrochloric acid (100 ml), 5% aqueous sodium hydrogen carbonate solution (100 ml), and brine (100 ml), dried (MgSO₄), and the solvents removed in vacuo. The residue was purified by flash chromatography over silica eluting with ethyl acetate:heptane 1:4 to give (S)-1-benzoyl-2,5-dihydro-1H-pyrrole-2-carboxylic acid 1-methyl ester (108) as a colourless oil (2.05 g, 73%). TLC (R_{f}=0.25, EtOAc:heptane 1:1); HPLC-MS 232.1, [M+H]⁺, 254.0 [M+Na]⁺, 485.1 [2M+Na]⁺; OH (500 MHz, CDCl₃) 3.78 (3H, s, OCH₃), 4.15-4.22 (1H, m, H-5), 4.41-4.47 (1H, m, H-5), 5.45-5.49 (1H, m, H-2), 5.83-5.95 (2H, m, H-3 and H-4), 7.36-7.58 (5H, aromatics); Sc (125 MHz, CDCl₃) δ2.50 (OCH₃), 55.90 (C-5), 66.39 (C-2), 124.89, 127.01, 128.39, 128.41, 128.60 and 130.24 (C-3, C-4 and aromatic CH), 135.86 (aromatic quaternary), 169.73 and 170.16 (CHC═O and NC═OPh).

### Preparation of (S)-(2-hydroxymethyl-2,5-dihydropyrrole-1-yl)phenyl methanone (109)

Methanol (0.71 ml) followed by a solution of (S)-1-benzoyl-2,5-dihydro-1H-pyrrole-2-carboxylic acid 1-methyl ester (108 (2.05 g, 8.9 mmol) in THF (37 ml) were added dropwise to a stirred suspension of lithium borohydride (390 mg, 17.7 mmol) in THF (13 ml). The mixture was stirred for 1 hour then water (5 ml) was carefully added. The product was extracted into tert-butyl methyl ether (3×50 ml), then the combined-organic layers dried (MgSO₄), and the solvents removed in vacuo to give (S)-(2-hydroxymethyl-2,5-dihydropyrrole-1-yl)phenylmethanone (109) as a pale yellow oil (1.68 g, 93%) which was used without further purification. TLC (R_{f}=0.1, EtOAc:heptane 1:1), HPLC-MS 204.1 [M+H]; d_{H} (500 MHz, CDCl₃) 1.65-1.95 (1H, br. s, CH₂OH), 3.72 (1H, dd J=11.4 and 7.1 Hz, CH₂OH), 3.90 (1H, dd J=11.6 and 2.1 Hz, CH₂OH), 4.11-4.16 (1H, m, H-5), 4.27-4.38 (1H, m, H-5), 5.17-5.21 (1H, m, H-2), 5.74-5.82 (2H, m, H-3 and H-4), 7.38-7.53 (5H, aromatics); d_{C} (125 MHz, CDCl₃) δ6.76 (C-5), 66.44 (CH₂OH), 68.68 (C-2), 126.25, 126.67, 126.93, 126.95, 128.50 and 130.20 (C-3, C-4 and aromatic CH), 136.21 (aromatic quaternary), 172.12 (NC═OPh).

### Preparation of (S)-methanesulfonic acid 1-benzoyl-2,5-dihydro-1H-pyrrol-2-ylmethyl ester (110)

Triethylamine (1.86 ml, 13.2 mmol) was added dropwise to a stirred solution of (S)-(2-hydroxymethyl-2,5-dihydropyrrole-1-yl)phenylmethanone (109) (1.68 g, 8.3 mmol) and methanesulfonyl chloride (0.96 ml, 12.4 mmol) in dichloromethane (30 ml) at 0° C. The mixture was stirred for 30 minutes at ambient temperature then washed with water (100 ml), and brine (100 ml), dried (Na₂SO₄), and the solvents removed in vacuo to give (S)-methanesulfonic acid 1-benzoyl-2,5-dihydro-1H-pyrrol-2-ylmethyl ester (110) as a pale yellow oil (1.88 g, 81%) which was used without further purification. HPLC-MS 282.0 [M+H]⁺, 585.1 [2M+Na]⁺.

### Preparation of (S)-(2-azidomethyl-2,5-dihydropyrrol-1-yl)phenylmethanone (111)

Sodium azide (2.17 g, 33.4 mmol) was added to a stirred solution of (S)-methanesulfonic acid 1-benzoyl-2,5-dihydro-1H-pyrrol-2-ylmethyl ester (110) (1.88 g, 6.68 mmol) in DMF (50 ml). The reaction mixture was stirred at 110° C. for 1 hour. The solvent was removed in vacuo then the product was extracted into ethyl acetate (100 ml), washed with water (100 ml) and brine (100 ml), dried (MgSO₄), and the solvents removed in vacuo. The residue was purified by flash chromatography over silica eluting with heptane:ethyl acetate 9:1 to give (S)-(2-azidomethyl-2,5-dihydropyrrol-1-yl)phenylmethanone (111) as a pale yellow oil (0.96 g, 63%). TLC (R_{f}=0.40, heptane:ethyl acetate 1:1), analytical HPLC Rₜ=13.943 min, HPLC-MS 229.1 [M+H]⁺, 251.1 [M+Na]⁺, 479.1 [2M+Na]⁺.

### Preparation of (S)-(2-aminomethyl-2,5-dihydro-pyrrol-1-yl)phenyl methanone (112)

Triphenylphosphine (1.65 g, 6.30 mmol) was added to a stirred solution of (S)-(2-azidomethyl-2,5-dihydropyrrol-1-yl)phenylmethanone (111) (0.96 g, 4.2 mmol) in THF (87 ml) containing water (1 ml). The mixture was stirred at 50° C. for 24 hours then the solvents were removed in vacuo. The residue was purified by flash chromatography over silica eluting with ethyl acetate:heptane 1:9 then ethyl acetate:methanol 4:1 mixtures to give (S)-(2-aminomethyl-2,5-dihydro-pyrrol-1-yl)phenylmethanone (112) as a pale yellow oil (0.77 g, 91%). TLC (R_{f}=0.1, chloroform:methanol 9:1), analytical HPLC with UV peaks at Rₜ=6.705 and 7.943 min; HPLC-MS 203.1 [M+H]⁺, 427.1 [2M+Na]⁺. d_{H} (500 MHz, D₆-DMSO) 1.30-2.10 (2H, br. s, CH₂NH₂), 2.84 (2H, m, CH₂NH₂), 3.87 (1H, dd, J=14.9 and 1.0 Hz, H-5), 4.26-4.35 (1H, m, H-5), 4.84 (1H, m, H-2), 5.83-6.03 (2H, m, H-4 and H-5), 7.39-7.58 (5H, aromatics); d_{C} (125 MHz, D₆-DMSO) 44.25 (CH₂NH₂), 56.96 (C-5), 67.20 (C-2), 126.50, 126.62, 126.83, 127.51, 128.63, 128.84, 128.96, 129.52, 129.81 and 130.22 (C-3, C-4 and aromatic CH), 137.48 (aromatic quaternary), 169.44 (NC═O).

### Preparation of (S)-N-(1-benzoyl-2,5-dihydro-1H-pyrrol-2-ylmethyl)hydrazine Carboxylic acid allyl ester (113)

3-Phenyloxaziridine-2-carboxylic acid allyl ester (prepared as above, 1.95 g, 9.5 mmol) was added to a stirred solution of (S)-(2-aminomethyl-2,5-dihydro-pyrrol-1-yl)phenylmethanone (112) (0.64 g, 3.17 mmol) in dichloromethane (10 ml). The mixture was stirred for 16 hours then the solvents were removed in vacuo. The oily residue was purified by flash chromatography over silica eluting with ethyl acetate:heptane mixtures 1:9 to 1:4 to give (S)-N-(1-benzoyl-2,5-dihydro-1H-pyrrol-2-ylmethyl)hydrazinecarboxylic acid allyl ester (113) as a pale yellow oil (0.26 g, 27%). TLC (R_{f}=0.45, heptane:ethyl acetate 1:1), HPLC-MS 302.1 [M+H]⁺, 324.1 [M+Na]⁺, 625.1 [2M+Na]⁺.

### Preparation of (S)-N′-(1-benzoyl-2,5-dihydro-1H-pyrrol-2-ylmethyl)-N′-tert-butoxycarbonyl-hydrazinecarboxylic acid allyl ester (114)

Three portions of Boc anhydride (each 1.84 g, 8.1 mmol) were added at one hour intervals to a stirred solution of (S)-N-(1-benzoyl-2,5-dihydro-1H-pyrrol-2-ylmethyl)hydrazinecarboxylic acid allyl ester (113) (0.26 g, 0.85 mmol) in triethylamine:methanol (1:9, 10 ml). The mixture was stirred at 60° C. for 3 hours then the solvents were removed in vacuo. The oily residue was purified by flash chromatography over silica eluting with ethyl acetate:heptane mixtures 0:1 to 1:4 to give (S)-N-(1-benzoyl-2,5-dihydro-1H-pyrrol-2-ylmethyl)-N′-tert-butoxycarbonyl-hydrazinecarboxylic acid allyl ester (114) as a pale yellow oil (96 mg, 280%). TLC (R_{f}=0.25, heptane:ethyl acetate 1:1), analytical HPLC Rₜ=6.025 min; HPLC-MS 402.1 [M+H]⁺, 825.1 [2M+Na]⁺.

### Preparation of (S)-N-(1-benzoyl-2,5-dihydro-1H-pyrrol-2-ylmethyl)hydrazine carboxylic acid tert-butyl ester (115)

Tetrakis(triphenylphosphine)palladium(0) (7.5 mg, 0.0065 mmol) was added to a stirred solution of (S)-N′-(1-benzoyl-2,5-dihydro-1H-pyrrol-2-ylmethyl)-N′-tert-butoxycarbonyl-hydrazinecarboxylic acid allyl ester (114) (131 mg, 0.326 mmol) in dichloromethane (5 ml) under an atmosphere of argon. Phenylsilane (81 μl, 0.65 mmol) was then added dropwise over two minutes. The solution was stirred for 1 hour then the solvents were removed in vacuo. The oily residue was purified by flash chromatography over silica eluting with heptane:tert-butyl methyl ether 9:1 to 0:1 mixtures to give (S)-N-(1-benzoyl-2,5-dihydro-1H-pyrrol-2-ylmethyl)hydrazine carboxylic acid tert-butyl ester (115) as a brown oil (77 mg, 74%). TLC (R_{f}=0.35, heptane:ethyl acetate 1:1), analytical HPLC Rₜ=12.738 min; HPLC-MS 218.1 [M+2H−Boc]⁺, 262.1 [M+2H−Bu]⁺, 318.1 [M+H]⁺, 635.3 [2M+H]⁺, 657.2 [2M+Na]⁺.

### Preparation of N′-((2S)-2-allyloxycarbonylamino-4-methylpentanoyl)-N-((2S)-1-benzoyl-2,5-dihydro-1H-pyrrol-2-ylmethyl)hydrazinecarboxylic acid tert-butyl ester (116)

Alloc-Leu-F (prepared as above, 55 mg, 0.26 mmol) was added to a stirred solution of (S)-2-N-(1-benzoyl-2,5-dihydro-1H-pyrrol-2-ylmethyl)hydrazine carboxylic acid tert-butyl ester (115) (77 mg, 0.24 mmol) in dimethylformamide (1.5 ml) under an atmosphere of nitrogen. The solution was stirred for 4.5 hours then the solvents were removed in vacuo. The residue was purified by flash chromatography over silica eluting with ethyl acetate:heptane mixtures 1:9 to 2:3 to give N′-((2S)-2-allyloxycarbonylamino-4-methylpentanoyl)-N-((2S)-1-benzoyl-2,5-dihydro-1H-pyrrol-2-ylmethyl)hydrazinecarboxylic acid tert-butyl ester (116) as a viscous oil (83 mg, 67%). TLC (Single spot, R_{f}=0.45, EtOAc:heptane 1:1), analytical HPLC Rₜ=19.157 min; HPLC-MS 415.1 [M+2H−Boc]⁺, 459.1 [M+2H−Bu]⁺, 515.2 [M+H]⁺.

### Preparation of N′-((2S)-2-amino-4-methylpentanoyl)-N-((2S)-1-benzoyl-2,5-dihydro-1H-pyrrol-2-ylmethyl)hydrazinecarboxylic acid tert-butyl ester (117)

Dichloromethane (1.5 ml) followed by phenylsilane (39 μl, 0.32 mmol) were consecutively added with stirring under an atmosphere of nitrogen to a mixture of tetrakistriphenylphosphine palladium(0) (3.7 mg, 0.003 mmol) and N′-((2S)-2-allyloxycarbonylamino-4-methylpentanoyl)-N-((2S)-1-benzoyl-2,5-dihydro-1H-pyrrol-2-ylmethyl)hydrazinecarboxylic acid tert-butyl ester (116) (82 mg, 0.16 mmol). The solution was stirred for 1.75 hours then purified by flash chromatography over silica eluting with methanol:dichloromethane mixtures 1:99 to 5:95 to give N′-((2S)-2-amino-4-methylpentanoyl)-N-((2S)-1-benzoyl-2,5-dihydro-1H-pyrrol-2-ylmethyl) hydrazinecarboxylic acid tert-butyl ester (117) as a colourless oil (57 mg, 83%). TLC (Single spot, R_{f}=0.45, MeOH dichloromethane 6:94), analytical HPLC Rt=14.217 min; HPLC-MS 431.1 [M+H]⁺, 861.3 [2M+H]⁺, 883.3 [2M+Na]⁺.

### Preparation of N-((2S)-1-benzoyl-2,5-dihydro-1H-pyrrol-2-ylmethyl)-N′-[(2S)-2-(4-tert-butylbenzoylamino)-4-methylpentanoyl]hydrazinecarboxylic acid tert-butyl ester (118)

4-Methylmorpholine (28.5 μl, 0.26 mmol) was added to a solution of HBTU (49 mg, 0.13 mmol), 1-hydroxybenzotriazole monohydrate (20 mg, 0.13 mmol) and 4-(tert-butyl)benzoic acid (23 mg, 0.13 mmol) in dimethylformamide (1.5 ml). The solution was stood for 5 minutes then added to N′-((2S)-2-amino-4-methylpentanoyl)-N-((2S)-1-benzoyl-2,5-dihydro-1H-pyrrol-2-ylmethyl) hydrazine carboxylic acid tert-butyl ester (117) (56 mg, 0.13 mmol). The mixture was stirred for 1.5 hour then the solvents were removed in vacuo (water bath temperature<33° C.). The residue was dissolved in dichloromethane (15 ml) then washed with pH 3 hydrochloric acid (5 ml), saturated aqueous sodium hydrogen carbonate solution (5 ml) and brine (5 ml), dried (Na₂SO₄), and the solvents removed in vacuo. The yellow residue (102 mg) was purified by flash chromatography over silica eluting with ethyl acetate:heptane mixtures 1:9 to 1:1 to give N-((2S)-1-benzoyl-2,5-dihydro-1H-pyrrol-2-ylmethyl)-N′-[(2S-2-(4-tert-butylbenzoylamino)-4-methylpentanoyl]hydrazinecarboxylic acid tert-butyl ester (118) as a white solid (52 mg, 68%). TLC (R_{f}=0.46, EtOAc:heptane 1:1), analytical HPLC Rₜ=22.310 min; HPLC-MS 491.2 [M+2H−Boc]⁺, 591.2 [M+H]⁺.

### Preparation of N-((2S)-3-benzoyl-6-oxa-3-aza-bicyclo[3.1.0]hex-2-ylmethyl)-N′-[(2S)-2-(4-tert-butylbenzoylamino)-4-methylpentanoyl]hydrazine carboxylic acid tert-butyl ester (119)

A solution of meta-chloroperoxybenzoic acid (57-86%, 210 mg, ˜0.86 mmol) in dichloromethane (1.25 ml) was added to N-((2S)-1-benzoyl-2,5-dihydro-1H-pyrrol-2-ylmethyl)-N′-[(2S)-2-(4-tert-butylbenzoylamino)-4-methylpentanoyl]hydrazine carboxylic acid tert-butyl ester (118) (51 mg, 0.086 mmol) under an atmosphere of nitrogen. The solution was stirred for 20 hours then dichloromethane (15 ml) was added and the mixture washed with 10% aqueous sodium hydroxide solution (10 ml). The aqueous layer was extracted with dichloromethane (5 ml) then the combined organic layers washed with 10% aqueous sodium hydroxide solution (10 ml), dried (Na₂SO₄), and the solvents removed in vacuo. The yellow oily residue was purified by flash chromatography over silica eluting with ethyl acetate:heptane mixtures _{1:9} to 1:1 to give N-((2S)-3-benzoyl-6-oxa-3-aza-bicyclo[3.1.0]hex-2-ylmethyl)-N′-[(2S)-2-(4-tert-butylbenzoylamino)-4-methylpentanoyl]hydrazinecarboxylic acid tert-butyl ester (119) as a white solid (27.5 mg, 52%). TLC (R_{f}=0.25, EtOAc:heptane 1:1), analytical HPLC Rₜ=21.769 min; HPLC-MS 507.2 [M+2H−Boc]⁺, 607.2 [M+H]⁺.

### Preparation of (3aR,6S,6aS)-4-benzoyl-1-[(2S-2-(4-tert-butylbenzoylamino)-4-methylpentanoyl]-6-hydroxyhexahydropyrrolo[3,2-c]pyrazole-2-carboxylic acid tert-butyl ester (106)

A solution of N-((2S)-3-benzoyl-6-oxa-3-aza-bicyclo[3.1.0]hex-2-ylmethyl)-N′-[(2S)-2-(4-tert-butylbenzoylamino)-4-methylpentanoyl]hydrazinecarboxylic acid tert-butyl ester (119) (26 mg, 0.043 mmol) in acetonitrile (2.5 ml) was added to potassium carbonate (150 mg, 1.09 mmol). The suspension was placed under an atmosphere of nitrogen then heated at 60° C. whilst stirring for 3.25 hours before being allowed to cool to ambient temperature. The suspension was filtered and the collected solid washed with acetonitrile (20 ml), then the filtrate was concentrated in vacuo. The residue was dissolved in dichloromethane (15 ml), washed with water (10 ml), dried (Na₂SO₄), and the solvents removed in vacuo. The residue was purified by flash chromatography over silica eluting with ethyl acetate heptane mixtures 1:4 to 1:1 to give (3aR,6S,6aS)-4-benzoyl-1-[(2S)-2-(4-tert-butylbenzoylamino)-4-methylpentanoyl]-6-hydroxyhexahydropyrrolo[3,2-c]pyrazole-2-carboxylic acid tert-butyl ester (106) as a white solid (16.2 mg, 62%). TLC (R_{f}=0.30, EtOAc:heptane 3:2), analytical HPLC Rₜ=22.013 min; HPLC-MS 278.1, 507.2 [M+2H−Boc]⁺, 607.2 [M+H]⁺.

EXAMPLES 249b and 249c were prepared from the intermediate (2S)-2-[N′-((2S)-2-amino-4-methylpentanoyl)-N-tert-butoxycarbonylhydrazinomethyl]-2,5-dihydro pyrrole-1-carboxylic acid benzyl ester (101) following Scheme 25;

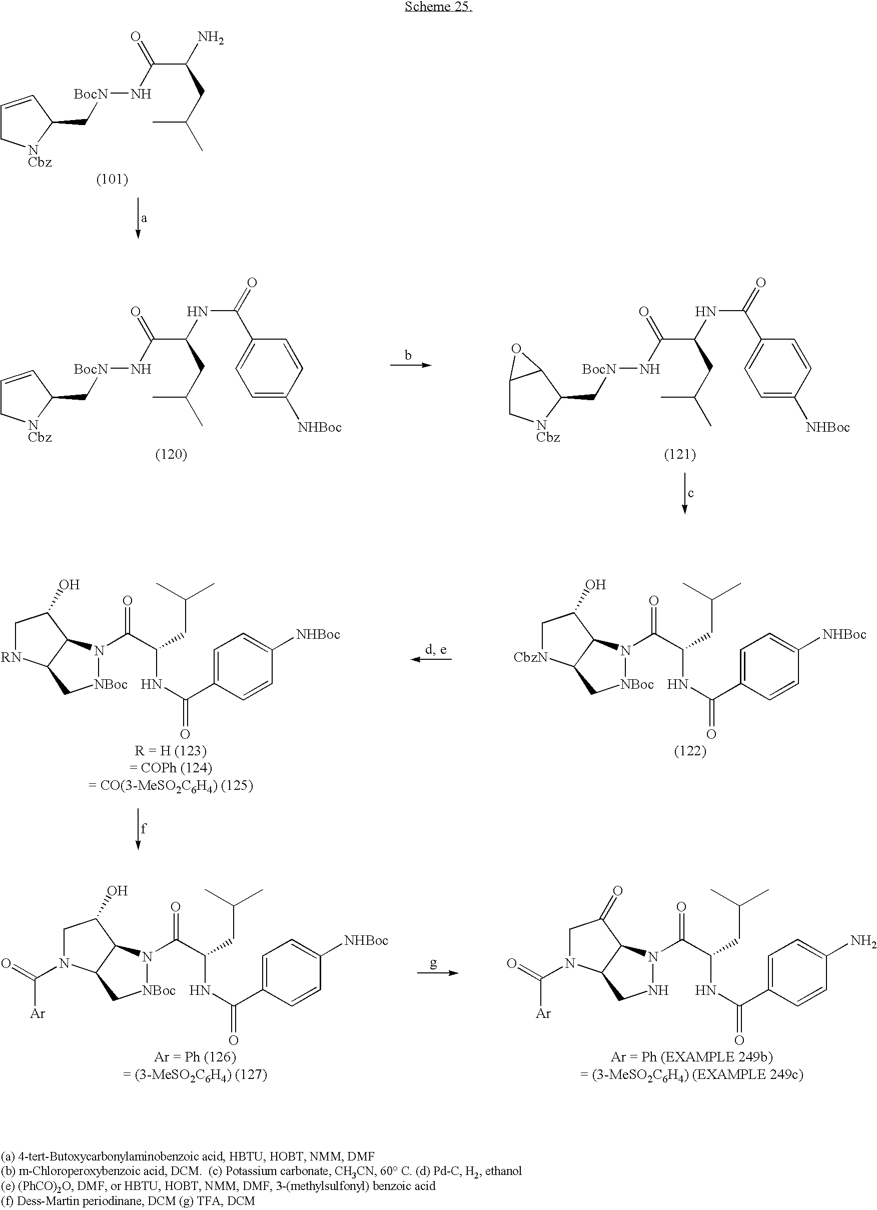

### Preparation of (2S)-2-{N-tert-butoxycarbonyl-N′-[(2S)-2-(4-tert-butoxycarbonylaminobenzoylamino)-4-methylpentanoyl]-hydrazinoyl methyl}-2,5-dihydro pyrrole-1-carboxylic acid benzyl ester (120)

4-Methylmorpholine (44.7 μl, 0.407 mmol) was added to a solution of HBTU (77 mg, 0.204 mmol), 1-hydroxybenzotriazole monohydrate (31 mg, 0.204 mmol) and 4-(tert-butoxycarbonylamino)benzoic acid (48 mg, 0.204 mmol) in dimethylformamide (2.0 ml). The solution was stood for 5 minutes then added to (2S)-2-[N′-((2S)-2-amino-4-methylpentanoyl)-N-tert-butoxycarbonylhydrazino methyl]-2,5-dihydropyrrole-1-carboxylic acid benzyl ester (101) (94 mg, 0.204 mmol). The mixture was stirred for 1 hour 35 minutes then the solvents were removed in vacuo (water bath temperature<37° C.). The residue was dissolved in dichloromethane (15 ml) then washed with pH 3 hydrochloric acid (5 ml), saturated aqueous sodium hydrogen carbonate solution (5 ml) and brine (5 ml), dried (Na₂SO₄), and the solvents removed in vacuo. The residue (166 mg) was purified by flash chromatography over silica eluting with ethyl acetate:heptane mixtures 1:9 to 4:6 to give (2S)-2-{N-tert-butoxycarbonyl-N′-[(2S)-2-(4-tert-butoxycarbonylaminobenzoylamino)-4-methylpentanoyl]-hydrazinoylmethyl}-2,5-dihydropyrrole-1-carboxylic acid benzyl ester (120) as a white solid (116 mg, 84%). TLC (R_{f}=0.48, EtOAc:heptane 1:1), analytical HPLC Rₜ=22.296 min; HPLC-MS 580.4 [M+2H−Boc]⁺, 680.4 [M+H]⁺.

### Preparation of (2S)-2-{N-tert-butoxycarbonyl-N′-[(2S)-2-(4-tert-butoxy carbonylaminobenzoylamino)-4-methylpentanoyl]-hydrazinomethyl}-6-oxa-3-aza-bicyclo [3.1.0]hexane-3-carboxylic acid benzyl ester (121)

A solution of meta-chloroperoxybenzoic acid (57-86%, 411 mg, ˜1.7 mmol) in dichloromethane (2.5 ml) was added to (2S)-2-{N-tert-butoxycarbonyl-N′-[(2S)-2-(4-tert-butoxycarbonylaminobenzoylamino)-4-methylpentanoyl]-hydrazinoyl methyl}-2,5-dihydropyrrole-1-carboxylic acid benzyl ester (120) (115 mg, 0.169 mmol) under an atmosphere of nitrogen. The solution was stirred for 20 hours then dichloromethane (15 ml) was added and the solution twice washed with a mixture of water (5 ml) and aqueous saturated sodium hydrogen carbonate solution (5 ml), dried (Na₂SO₄), and the solvents removed in vacuo. The residue (127 mg) was purified by flash chromatography over silica eluting with ethyl acetate:heptane mixtures 1:9 to 1:1 to give (2S)-2-{N-tert-butoxycarbonyl-N′-[(2S)-2-(4-tert-butoxycarbonylaminobenzoylamino)-4-methylpentanoyl]-hydrazinomethyl}-6-oxa-3-aza-bicyclo[3.1.0] hexane-3-carboxylic acid benzyl ester (121) as a white solid (63.7 mg, 54%). TLC (R_{f}=0.25, EtOAc:heptane 1:1), analytical HPLC Rₜ=21.723 min; HPLC-MS 596.4 [M+2H−Boc]⁺, 696.4 [M+H]⁺, 718.4 [M+Na]⁺.

### Preparation of (3aR,6S,6aS)-1-[(2S)-2-(4-tert-butoxycarbonylamino benzoylamino)-4-methylpentanoyl]-6-hydroxyhexahydropyrrolo[3,2c pyrazole-2,4-dicarboxylic acid 4-benzyl ester 2-tert-butyl ester (122)

A solution of (2S)-2-{N-tert-butoxycarbonyl-N′-[(2S)-2-(4-tert-butoxycarbonyl aminobenzoylamino)-4-methylpentanoyl]-hydrazinomethyl}-6-oxa-3-aza-bicyclo [3.1.0]hexane-3-carboxylic acid benzyl ester (121) (62.2 mg, 0.090 mmol) in acetonitrile (3.0 ml) was added to potassium carbonate (300 mg, 2.17 mmol). The suspension was placed under an atmosphere of nitrogen then heated at 60° C. whilst stirring for 3 hours before being allowed to cool to ambient temperature. The suspension was filtered then the filtrate concentrated in vacuo. The product was extracted into dichloromethane (15 ml) then washed with water (5 ml), dried (Na₂SO₄), and the solvents removed in vacuo. The residue was purified by flash chromatography over silica eluting with ethyl acetate:heptane mixtures 1:4 to 3:2 to give (3aR,6S,6aS)-1-[(2S)-2-(4-tert-butoxycarbonylaminobenzoylamino)-4-methylpentanoyl]-6-hydroxyhexahydropyrrolo[3,2-c]pyrazole-2,4-dicarboxylic acid 4-benzyl ester 2-tert-butyl ester (122) as a white solid (36.7 mg, 59%). TLC (R_{f}=0.28, EtOAc:heptane 1:1), analytical HPLC Rₜ=22.528 min; HPLC-MS 333.3, 596.4 [M+2H−Boc]⁺, 640.4 [M+2H−Bu]⁺, 696.4 [M+H]⁺.

### Preparation of (3aR,6S,6aS)-1-[(2S)-2-(4-tert-butoxycarbonylamino benzoylamino)-4-methylpentanoyl]-6-hydroxyhexahydropyrrolo[3,2-c] pyrazole-2-carboxylic acid tert-butyl ester (123)

Under an atmosphere of nitrogen ethanol (2.6 ml) was added to a mixture of (3aR,6S,6aS)-1-[(2S)-2-(4-tert-butoxycarbonylaminobenzoylamino)-4-methyl pentanoyl]-6-hydroxyhexahydropyrrolo[3,2-c]pyrazole-2,4-dicarboxylic acid 4 benzyl ester 2-tert-butyl ester (122) (35.2 mg, 0.051 mmol) and 10% palladium on charcoal (20 mg) whilst stirring. The nitrogen was replaced by hydrogen then stirring continued for 1 hour. The hydrogen was replaced by nitrogen then the mixture filtered through celite. The filter cake was washed with ethanol (20 ml) then the filtrate concentrated in vacuo. The residue (28.3 mg, 100%) was used without further purification. Analytical HPLC Rₜ=17.437 min; HPLC-MS 462.3 [M+2H−Boc]⁺, 562.4 [M+H]⁺ for (3aR,6S,6aS)-1-[(2S)-2-(4-tert-butoxycarbonylaminobenzoylamino)-4-methylpentanoyl]-6-hydroxyhexahydro pyrrolo[3,2-c]pyrazole-2-carboxylic acid tert-butyl ester (123).

### Preparation of (3aR,6S,6aS)-4-benzoyl-1-[(2S)-2-(4-tert-butoxycarbonyl aminobenzoylamino)-4-methylpentanoyl]hydroxyhexahydropyrrolo[3,2-c]pyrazole-2-carboxylic acid tert-butyl ester (124)

Benzoic anhydride (5.3 mg, 0.024 mmol), dimethylformamide (0.275 ml) then 4-methylmorpholine (5.2 μd, 0.047 mmol) were added consecutively to (3aR,6S,6aS)-1-[(2S)-2-(4-tert-butoxycarbonylaminobenzoylamino)-4-methylpentanoyl]-6-hydroxy hexahydropyrrolo[3,2-c]pyrazole-2-carboxylic acid tert-butyl ester (123) (12.6 mg, 0.0225 mmol, prepared as above). The solution was stirred for 90 minutes then the majority of solvents were removed in vacuo. The residue was dissolved in ethyl acetate (10 ml), then washed with saturated aqueous sodium hydrogen carbonate solution (5 ml), pH 3 hydrochloric acid (5 ml) and brine (5 ml), dried (Na₂SO₄), and the solvents removed in vacuo. The solid white residue (15.4 mg, 100%) was used without further purification. Data for (3aR,6S,6aS)-4-benzoyl-1-[(2S)-2-(4-tert-butoxycarbonylaminobenzoylamino)-4-methyl pentanoyl]-6-hydroxyhexahydro pyrrolo[3,2-c]pyrazole-2-carboxylic acid tert-butyl ester (124): TLC (R_{f}=0.25, EtOAc:heptane 2:1), analytical HPLC Rₜ=20.787 min; HPLC-MS 333.3, 566.4 [M+2H−Boc]⁺, 666.4 [M+H]⁺.

### Preparation of (3aR,6aS)-4-benzoyl-1-[(2S)-2-(4-tert-butoxycarbonylamino benzoylamino)-4-methylpentanoyl]6-oxo-hexahydropyrrolo[3,2-c]pyrazole-2-carboxylic acid tert-butyl ester (126)

A solution of Dess-Martin periodinane (48 mg, 0.113 mmol) in dichloromethane (1.1 ml) was added to (3aR,6S,6aS)-4-benzoyl-1-[(2S)-2-(4-tert-butoxycarbonyl aminobenzoylamino)-4-methylpentanoyl]-6-hydroxyhexahydropyrrolo[3,2-c]pyrazole-2-carboxylic acid tert-butyl ester (124) (15.4 mg, 0.0225 mmol, prepared as above) under an atmosphere of nitrogen. The mixture was stirred for 3 hours then purified by flash chromatography over silica eluting with ethyl acetate:heptane mixtures 1:4 to 1:1 to give (3aR,6aS)-4-benzoyl-1-[(2S)-2-(4-tert-butoxycarbonylaminobenzoylamino)-4-methylpentanoyl]-6-oxo-hexahydro pyrrolo[3,2-c]pyrazole-2-carboxylic acid tert-butyl ester (126) as an off-white solid (9.7 mg, 65%). TLC (R_{f}=0.28, EtOAc:heptane 2:1), analytical HPLC broad peak Rₜ=20.05-22.80 min; HPLC-MS single broad main UV peak 333.2, 664.4 [M+H]⁺, 682.4 [M+H₂O+H]⁺.

### Preparation of (3aR,6aS)-4-amino-N-[(1S)-1-(4-benzoyl-oxo-hexahydro pyrrolo[3,2-c]pyrazole-1-carbonyl)-3-methylbutyl]benzamide

### EXAMPLE 249b

Trifluoroacetic acid (0.05 ml) was added to (3aR,6aS)-4-benzoyl-1-[(2S)-2-(4-tert-butoxycarbonylaminobenzoylamino)-4-methylpentanoyl]-6-oxo-hexahydro pyrrolo[3,2-c]pyrazole-2-carboxylic acid tert-butyl ester (126) (6.4 mg, 9.7 μmol) under an atmosphere of nitrogen. The solution was stirred for 2.5 hours then diluted with dichloromethane (1 ml) and cautiously added to saturated aqueous sodium hydrogen carbonate solution (1 ml). The dichloromethane was separated then washed with water (1 ml). The saturated aqueous sodium hydrogen carbonate solution was extracted with dichloromethane (0.5 ml) which was then washed with the water layer. The combined organic layers were dried (Na₂SO₄), and the solvents removed in vacuo to obtain (3aR,6aS)-4-amino-N-[1-(4-benzoyl-6-oxo-hexahydropyrrolo[3,2-c]pyrazole-1-carbonyl)-3-methylbutyl)benzamide (EXAMPLE 249b) as a red solid (0.72 mg, 16%). Analytical HPLC broad peak Rₜ=11.0-12.1 min; HPLC-MS broad UV peak 464.2 [M+H]⁺, 482.4 [M+H₂O+H]⁺, 949.3 [2M+Na]⁺, 967.4 [2M+H₂O+Na]⁺, 985.3 [2M+2H₂O+Na]⁺.

### Preparation of (3aR,6S,6aS)-1-[(2S)-2-(4-tert-butoxycarbonyl aminobenzoylamino)-4-methylpentanoyl]-6-hydroxy-4-(3-methanesulfonyl benzoyl)hexahydropyrrolo[3,2-]pyrazole-2-carboxylic acid tert-butyl ester (125)

4-Methylmorpholine (5.7 μl, 0.052 mmol) was added to a solution of HBTU (9.9 mg, 0.026 mmol), 1-hydroxybenzotriazole monohydrate (4.0 mg, 0.026 mmol) and 3-(methylsulfonyl)benzoic acid (5.2 mg, 0.026 mmol) in dimethylformamide (0.3 ml). The solution was stood for 5 minutes then added to (3aR,6S,6aS)-1-[(2S)-2-(4-tert-butoxycarbonylaminobenzoylamino)-4-methylpentanoyl]-6-hydroxyhexahydro pyrrolo[3,2-c]pyrazole-2-carboxylic acid tert-butyl ester (123) (14.7 mg, 0.026 mmol, prepared as above). The mixture was stirred for 2.5 hour then the solvents were removed in vacuo (water bath temperature<37° C.). The residue was dissolved in dichloromethane (10 ml) then washed with pH 3 hydrochloric acid (5 ml), saturated aqueous sodium hydrogen carbonate solution (5 ml) and brine (5 ml), dried (Na₂SO₄), and the solvents removed in vacuo. The pale yellow solid residue (19.7 mg, 100%) was used without further purification. Data for (3aR,6S,6aS)-1-[(2S)-2-(4-tert-butoxycarbonylaminobenzoylamino)-4-methylpentanoyl]-6-hydroxy-4-(3-methanesulfonylbenzoyl)hexahydropyrrolo [3,2-c]pyrazole-2-carboxylic acid tert-butyl ester (125): TLC (R_{f}=0.05, EtOAc:heptane 2:1), analytical HPLC Rₜ=19.945 min; HPLC-MS 333.3, 644.3 [M+2H−Boc]⁺, 688.3 [M+2H−Bu]⁺, 744.3 [M+H]⁺, 766.3 [M+Na]⁺.

### Preparation of (3aR,6aS)-1-[(2S)-2-(4-tert-butoxycarbonylaminobenzoyl amino)-4-methylpentanoyl]-4-(3-methanesulfonylbenzoyl)-6-oxohexahydro pyrrolo[3,2-c] pyrazole-2-carboxylic acid tert-butyl ester (127)

A solution of Dess-Martin periodinane (56 mg, 0.132 mmol) in dichloromethane (1.25 ml) was added to (3aR,6S,6aS)-1-[(2S)-2-(4-tert-butoxycarbonylamino benzoylamino)-4-methylpentanoyl]-6-hydroxy-4-(3-methanesulfonylbenzoyl) hexahydropyrrolo[3,2-c]pyrazole-2-carboxylic acid tert-butyl ester (125) (19.7 mg, 0.0262 mmol, prepared as above) under an atmosphere of nitrogen. The mixture was stirred for 4 hours then purified by flash chromatography over silica eluting with ethyl acetate:heptane mixtures 3:7 to 4:1 to give (3aR,6aS)-1-[(2S)-2-(4-tert-butoxycarbonylaminobenzoylamino)-4-methylpentanoyl]-4-(3-methanesulfonylbenzoyl)-6-oxo-hexahydropyrrolo[3,2-c]pyrazole-2-carboxylic acid tert-butyl ester (127) as an off-white solid (11.1 mg, 57%). TLC (R_{f}=0.10, EtOAc:heptane 2:1), analytical HPLC broad peak Rₜ=19.20-21.70 min; HPLC-MS single broad main UV peak 333.2, 642.3 [M+2H−Boc]⁺, 660.3 [M+2H+H₂O— Boc]⁺, 686.3 [M+2H−Bu]⁺, 704.2 [M+2H+H₂O— Bu]⁺, 742.3 [M+H]⁺, 760.3 [M+H₂O+H]⁺.

### Preparation of (3aR,6aS)-4-amino-N-{(1S)-1-[4-(3-methanesulfonylbenzoyl)-6-oxo-hexahydropyrrolo[3,2-c]pyrazole-1-carbonyl]-3-methylbutyl} benzamide

### EXAMPLE 249c

Trifluoroacetic acid (0.05 ml) was added to (3aR,6aS)-1-[(2S)-2-(4-tert-butoxycarbonylaminobenzoylamino)-4-methylpentanoyl]-4-(3-methanesulfonyl benzoyl)-6-oxo-hexahydropyrrolo[3,2-c]pyrazole-2-carboxylic acid tert-butyl ester (127) (4.93 mg, 6.7 μmol) under an atmosphere of nitrogen. The solution was stirred for 2.5 hours then diluted with dichloromethane (1 ml) and cautiously added to saturated aqueous sodium hydrogen carbonate solution (1 ml). The dichloromethane was separated then washed with water (1 ml). The saturated aqueous sodium hydrogen carbonate solution was extracted with dichloromethane (0.5 ml) which was then washed with the water layer. The combined organic layers were dried (Na₂SO₄), and the solvents removed in vacuo to obtain (3aR,6aS)-4-amino-N-{(1S)-1-[4-(3-methanesulfonylbenzoyl)-6-oxo-hexahydropyrrolo [3,2-c]pyrazole-1-carbonyl]-3-methylbutyl}benzamide (EXAMPLE 249c) as a red solid (0.66 mg, 18%). Analytical HPLC broad peak Rₜ=10.2-11.4 min; HPLC-MS broad UV peak 233.1, 542.2 [M+H]⁺, 560.2 [M+H₂O+H]⁺.

EXAMPLES 250 to 295 were prepared as detailed for EXAMPLES 1 and 119, substituting the appropriate carboxylic acids as required and are inhibitors of cathepsin S with Ki ranging from 10-5000 nM;

### EXAMPLE 250

### (3aR,6aS)-Thiophene-3-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

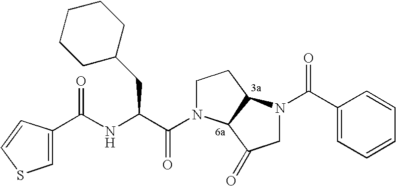

HPLC Rt=16.4-17.2 mins (>90%), HPLC-MS 494.2 [M+H]⁺, 1009.4 [2M+Na]⁺.

### EXAMPLE 251

### (3aR,6aS)-Thiophene-3-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

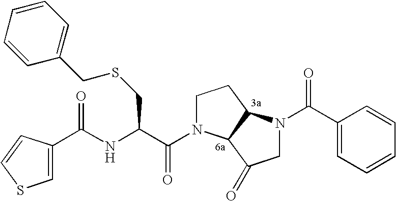

HPLC Rt=14.78 mins (>90%), HPLC-MS 534.1 [M+H]⁺.

### EXAMPLE 252

### (3aR,6aS)-Benzo[b]thiophene-2-carboxylic acid [(1R)-2-(4-benzoyl-6-oxo-hexahydro-pyrrolo[3,2-b]pyrrol-1-yl)-1-benzylsulfanylmethyl-2-oxo-ethyl]-amide

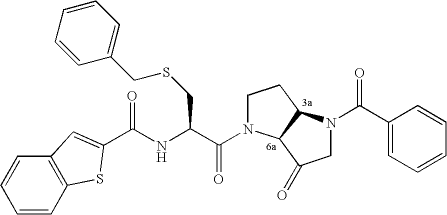

HPLC Rt=18.5-19.8 mins (>85%), HPLC-MS 584.1 [M+H]⁺.

### EXAMPLE 253

### (3aR,6aS)-Benzo[b]thiophene-3-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

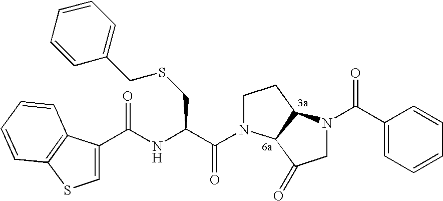

HPLC Rt=17.8-19.0 mins (>80%), HPLC-MS 584.2 [M+H]⁺.

### EXAMPLE 254

### (3aR,6aS)-Benzo[b]thiophene-3-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

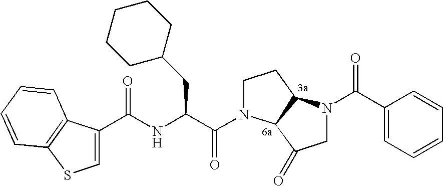

HPLC Rt=19.5-20.5 mins (>75%), HPLC-MS 544.1 [M+H]⁺.

### EXAMPLE 255

### (3aR,6aS)-Thiophene-3-carboxylic acid {(1S)-1-cyclohexylmethyl-2-[4-(naphthalene-1-carbonyl)-6-oxo-hexahydro-pyrrolo[3,2-b]pyrrol-1-yl]-2-oxo-ethyl}-amide

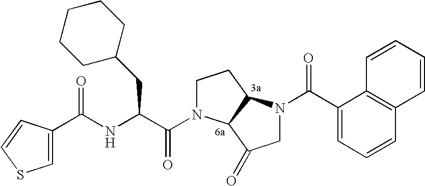

HPLC Rt=18.3-19.5 mins (>80%), HPLC-MS 544.1 [M+H]⁺.

### EXAMPLE 256

### (3aR,6aS)-Thiophene-3-carboxylic acid {(1S)-1-cyclohexylmethyl-2-[4-(naphthalene-2-carbonyl)-6-oxo-hexahydro-pyrrolo[3,2-b]pyrrol-1-yl]-2-oxo-ethyl}-amide

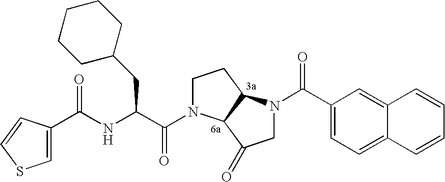

HPLC Rt=18.9-19.7 mins (>85%), HPLC-MS 544.1 [M+H]⁺.

### EXAMPLE 257

### (3aR,6aS)-Thiophene-3-carboxylic acid {(1S)-1-cyclohexylmethyl-2-oxo-2-[6-oxo-4-(4-[1,2,4]triazol-1-yl-benzoyl)-hexahydro-pyrrolo[3,2-b]pyrrole-1-yl]-ethyl}-amide

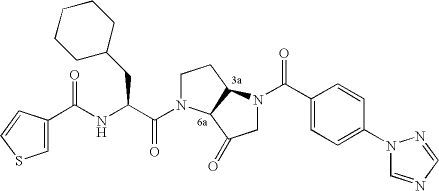

HPLC Rt=15.5-16.5 mins (>85%), HPLC-MS 561.2 [M+H]⁺.

### EXAMPLE 258

### (3aR,6aS)-Thiophene-3-carboxylic acid {(1S)-1-cyclohexylmethyl-2-oxo-2-[6-oxo-4-(5-phenyl-thiophene-2-carbonyl)-hexahydro-pyrrolo[3,2-b]pyrrole-1-yl]-ethyl}-amide

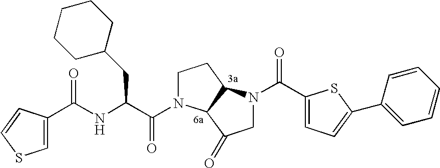

HPLC Rt=19.5-20.2 mins (>85%), HPLC-MS 576.1 [M+H]⁺.

### EXAMPLE 259

### (3aR,6aS)-N-[(1S)-2-(4-Benzoyl-6-oxo-hexahydro-pyrrolo[3,2-b]pyrrol-1-yl)-1-cyclohexylmethyl-2-oxo-ethyl]-3-phenoxy-benzamide

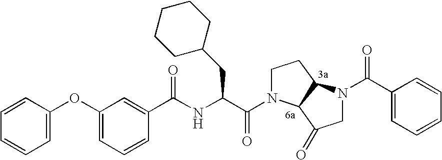

HPLC Rt=19.4-20.3 mins (>85%), —HPLC-MS 580.2 [M+H]⁺.

### EXAMPLE 260

### (3aR,6aS)-N-[(1R)-2-(4-Benzoyl-6-oxo-hexahydro-pyrrolo[3,2-b]pyrrol-1-yl)-1-benzylsulfanylmethyl-2-oxo-ethyl]-3-phenoxy-benzamide

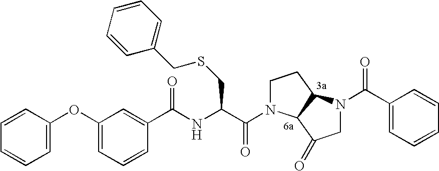

HPLC Rt=18.3-19.6 mins (>90%), HPLC-MS 620.2 [M+H]⁺.

### EXAMPLE 261

### (3aR,6aS)-Thiophene-3-carboxylic acid {(1R)-1-benzylsulfanyl methyl-2-[4-(2-methanesulfonyl-benzoyl)-6-oxo-hexahydro-pyrrolo[3,2-b]pyrrol-1-yl]-2-oxo-ethyl}-amide

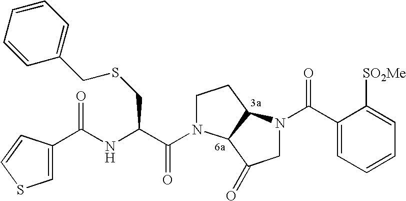

HPLC Rt=14.5-15.2 mins (>80%), HPLC-MS 612.0 [M+H]⁺.

### EXAMPLE 262

### (3aR,6aS)-Thiophene-3-carboxylic acid {(1R)-1-benzylsulfanyl methyl-2-(4-(3-methanesulfonyl-benzoyl)-6-oxo-hexahydro-pyrrolo[3,2-b]pyrrol-1-yl)-2-oxo-ethyl}-amide

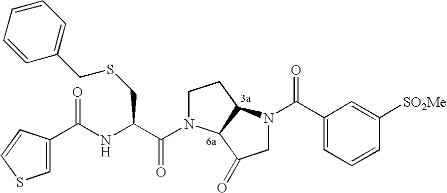

HPLC Rt=14.09 mins (>90%), HPLC-MS 612.1 [M+H]⁺.

### EXAMPLE 263

### (3aR,6aS)-Thiophene-3-carboxylic acid {(1R)-1-benzylsulfanyl methyl-2-[4-(2-nitro-benzoyl)-6-oxo-hexahydro-pyrrolo[3,2-b]pyrrole-1-yl]-2-oxo-ethyl}-amide

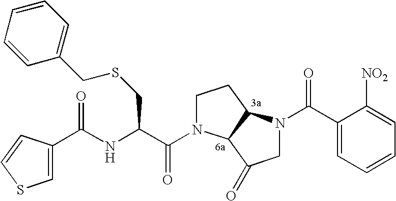

HPLC Rt=14.93 mins (>90%), HPLC-MS 579.0 [M+H]⁺.

### EXAMPLE 264

### (3aR,6aS)-Thiophene-3-carboxylic acid {(1R)-1-benzylsulfanyl methyl-2-[4-(3-nitro-benzoyl)-6-oxo-hexahydro-pyrrolo[3,2-b]pyrrole-1-yl]-2-oxo-ethyl}-amide

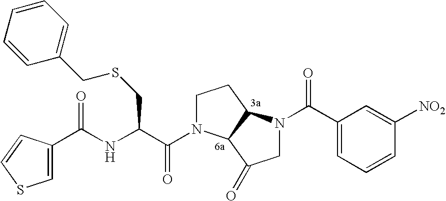

HPLC Rt=15.53 mins (>85%), HPLC-MS 579.0 [M+H]⁺.

### EXAMPLE 265

### (3aR,6aS)-Thiophene-3-carboxylic acid {(1R)-1-benzylsulfanyl methyl-2-[4-(4-nitro-benzoyl)-6-oxo-hexahydro-pyrrolo[3,2-b]pyrrole-1-yl]-2-oxo-ethyl}-amide

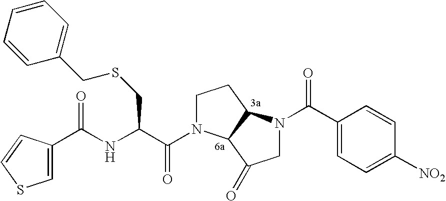

HPLC Rt=15.41 mins (>90%), HPLC-MS 579.1 [M+H]⁺.

### EXAMPLE 266

### (3aR,6aS)-Thiophene-3-carboxylic acid {(1R)-1-benzylsulfanyl methyl-2-[4-(hexahydro-2,5-methano-pentalene-3a-carbonyl)-6-oxo-hexahydro-pyrrolo[3,2-b]pyrrole-1-yl]-2-oxo-ethyl}-amide

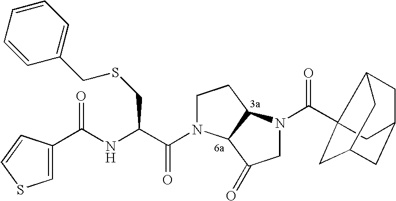

HPLC Rt=17.0-18.2 mins (>80%), HPLC-MS 578.1 [M+H]⁺.

### EXAMPLE 267

### (3aR,6aS)-Thiophene-3-carboxylic acid {(1R)-1-benzylsulfanyl methyl-2-[4-(4-hydroxy-benzoyl)-6-oxo-hexahydro-pyrrolo[3,2-b]pyrrol-1-yl]-2-oxo-ethyl}-amide

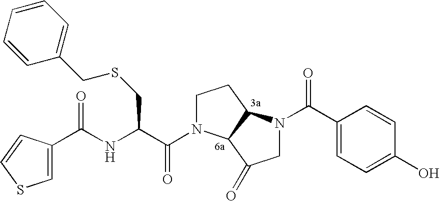

HPLC Rt=13.43 mins (>80%), HPLC-MS 550.0 [M+H]⁺.

### EXAMPLE 268

### (3aR,6aS)-Thiophene-3-carboxylic acid {(1R)-2-(4-(4-amino-benzoyl)-6-oxo-hexahydro-pyrrolo[3,2-b]pyrrol-1-yl]-1-benzylsulfanylmethyl-2-oxo-ethyl}-amide

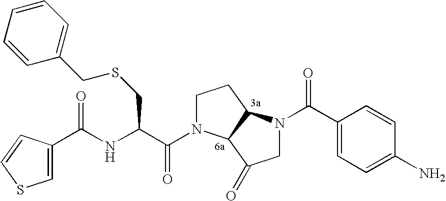

HPLC Rt=11.8-12.4 mins (>75%), HPLC-MS 549.1 [M+H]⁺.

### EXAMPLE 269

### (3aR,6aS)-N-[(1R)-2-(4-Benzoyl-6-oxo-hexahydro-pyrrolo[3,2-b]pyrrol-1-yl)-1-benzylsulfanylmethyl-2-oxo-ethyl]-2,2-diphenyl-acetamide

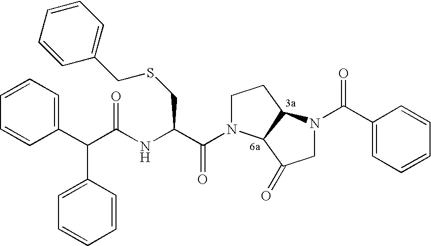

HPLC Rt=19.1-20.3 mins (>90%), HPLC-MS 618.2 [M+H]⁺.

### EXAMPLE 270

### (3aR,6aS)-N-[(1S)-2-(4-Benzoyl-6-oxo-hexahydro-pyrrolo[3,2-b]pyrrol-1-yl)-1-cyclohexylmethyl-2-oxo-ethyl]-2,2-diphenyl-acetamide

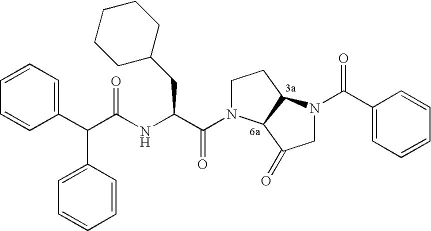

HPLC Rt=19.9-21.0 mins (>90%), HPLC-MS 578.3 [M+H]⁺.

### EXAMPLE 271

### (3aR,6aS)-N-{(1S)-1-[4-(Benzo[b]thiophene-3-carbonyl)-6-oxo-hexahydro-pyrrolo[3,2-b]pyrrole-1-carbonyl]-3-methyl-butyl}-2,2-diphenyl-acetamide

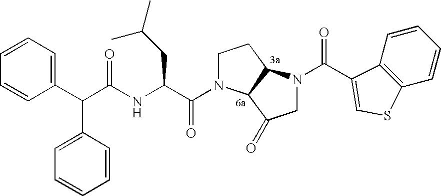

HPLC Rt=19.4-20.7 mins (>90%), HPLC-MS 594.2 [M+H]⁺.

### EXAMPLE 272

### (3aR,6aS)-Thiophene-3-carboxylic acid {(1R)-1-benzylsulfanyl methyl-2-oxo-2-[6-oxo-4-(thiophene-3-carbonyl)-hexahydro-pyrrolo[3,2-b]pyrrol-1-yl]-ethyl}-amide

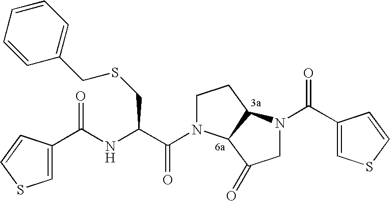

HPLC Rt=14.56 mins (>90%), HPLC-MS 540.0 [M+H]⁺.

### EXAMPLE 273

### (3aR,6aS)-Thiophene-3-carboxylic acid [(1R)-1-benzylsulfanyl methyl-2-(4-cyclohexanecarbonyl-6-oxo-hexahydro-pyrrolo[3,2-b]pyrrol-1-yl)-2-oxo-ethyl]-amide

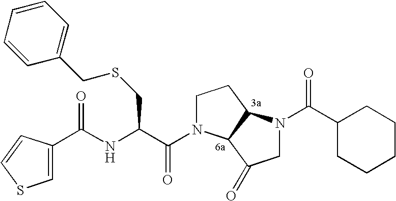

HPLC Rt=15.5-16.4 mins (>75%), HPLC-MS 540.1 [M+H]⁺.

### EXAMPLE 274

### (3aR,6aS)-Thiophene-3-carboxylic acid {(1R)-1-benzylsulfanyl methyl-2-[4-(2-methyl-benzoyl)-6-oxo-hexahydro-pyrrolo[3,2-b]pyrrole-1-yl]-2-oxo-ethyl}-amide

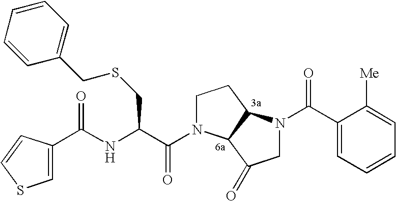

HPLC Rt=16.0-17.9 mins (>90%), HPLC-MS 548.1 [M+H]⁺.

### EXAMPLE 275

### (3aR,6aS)-Thiophene-3-carboxylic acid {(1R)-1-benzylsulfanyl methyl-2-[4-(3-methyl-benzoyl)-6-oxo-hexahydro-pyrrolo[3,2-b]pyrrol-1-yl]-2-oxo-ethyl}-amide

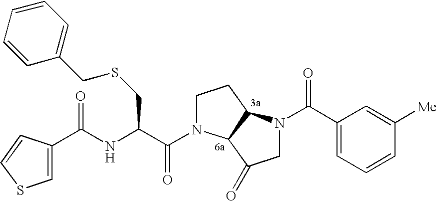

HPLC Rt=16.3-17.8 mins (>90%), HPLC-MS 548.1 [M+H]⁺.

### EXAMPLE 276

### (3aR,6aS)-Thiophene-3-carboxylic acid {(1R)-1-benzylsulfanyl methyl-2-[4-(4-methyl-benzoyl)-6-oxo-hexahydro-pyrrolo[3,2-b]pyrrole-1-yl]-2-oxo-ethyl}-amide

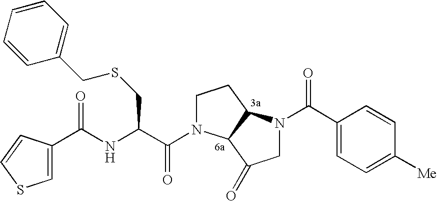

HPLC Rt=15.5-16.8 mins (>90%), HPLC-MS 548.1 [M+H]⁺.

### EXAMPLE 277

### (3aR,6aS)-Morpholine-4-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

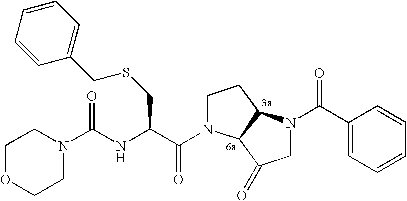

HPLC Rt=12.5-13.8 mins (>80%), HPLC-MS 537.1 [M+H]⁺.

### EXAMPLE 278

### (3aR,6aS)-3-Aminomethyl-N-[(1R)-2-(4-benzoyl-6-oxo-hexahydro-pyrrolo[3,2-b]pyrrole-1-yl)-1-benzylsulfanylmethyl-2-oxo-ethyl]-benzamide

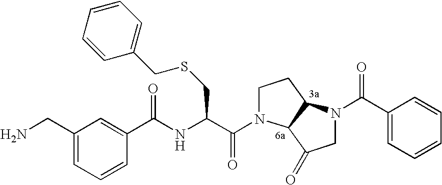

HPLC Rt=12.0-13.4 mins (>80%), HPLC-MS 557.2 [M+H]⁺.

### EXAMPLE 279

### (3aR,6aS)-N-{(1S)-1-Cyclohexylmethyl-2-[4-(2-methanesulfonyl-benzoyl)-6-oxo-hexahydro-pyrrolo[3,2-b]pyrrole-1-yl]-2-oxo-ethyl}-2,2-diphenyl-acetamide

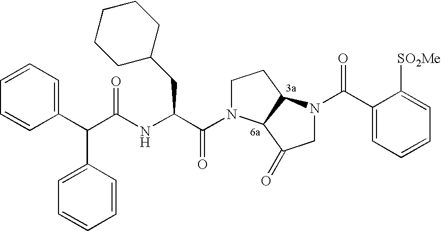

HPLC Rt=20.1-21.4 mins (>90%), HPLC-MS 656.2 [M # H)⁺.

### EXAMPLE 280

### (3aR,6aS)-Morpholine-4-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

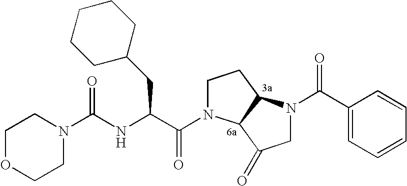

HPLC Rt=14.96 mins (>95%), HPLC-MS 497.2 [M+H]⁺, 515.2 [M+H+H₂O]⁺.

### EXAMPLE 281

### (3aR,6aS)-Benzothiazole-6-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

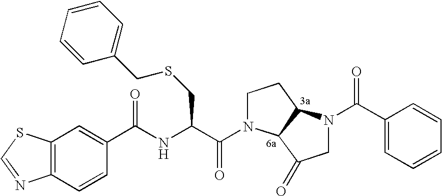

HPLC Rt=15.15 mins (>85%), HPLC-MS 585.1 [M+H]⁺, 603.1 [M+H+H₂O]⁺.

### EXAMPLE 282

### (3aR,6aS)-Thiophene-2-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

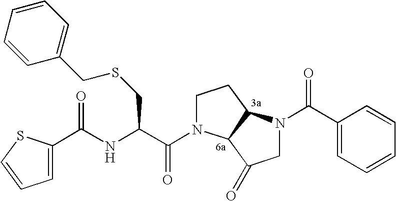

HPLC Rt=15.06 mins (>85%), HPLC-MS 534.0 [M+H]⁺.

### EXAMPLE 283

### (3aR,6aS)-Furan-2-carboxylic acid [(1R)-2-(4-benzoyl-6-oxo-hexahydro-pyrrolo[3,2-b]pyrrole-1-yl)-1-benzylsulfanylmethyl-2-oxo-ethyl)-amide

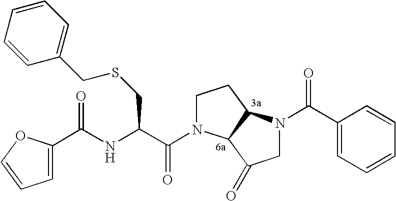

HPLC Rt=14.1-15.4 mins (>80%), HPLC-MS 518.1 [M+H]⁺, 536.1 [M+H+H₂O]⁺.

### EXAMPLE 284

### (3aR,6aS)-Furan-3-carboxylic acid [(1R)-2-(4-benzoyl-6-oxo-hexahydro-pyrrolo[3,2-b]pyrrole-1-yl)-1-benzylsulfanylmethyl-2-oxo-ethyl]-amide

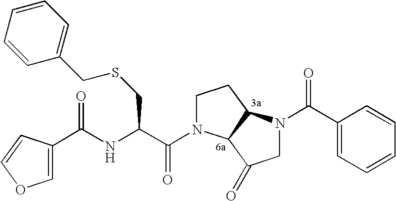

HPLC Rt=15.7-17.3 mins (>85%), HPLC-MS 518.1 [M+H]⁺.

### EXAMPLE 285

### (3aR,6aS)-N-[(1R)-2-(4-Benzoyl-6-oxo-hexahydro-pyrrolo[3,2-b] pyrrol-1-yl)-1-benzylsulfanylmethyl-2-oxo-ethyl]-benzamide

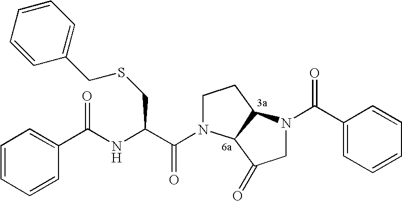

HPLC Rt=15.34 mins (>90%), HPLC-MS 528.1 [M+H]⁺.

### EXAMPLE 286

### (3aR,6aS)-Benzothiazole-6-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

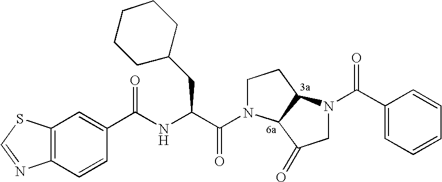

HPLC Rt=18.25 mins (>90%), HPLC-MS 5452 [M+H]⁺.

### EXAMPLE 287

### (3aR,6aS)-Thiophene-2-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

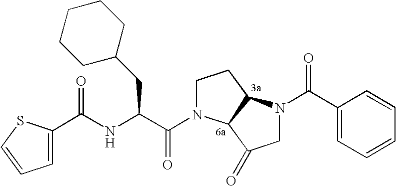

HPLC Rt=18.28 mins (>90%), HPLC-MS 494.1 [M+H]⁺.

### EXAMPLE 288

### (3aR,6aS)-Furan-2-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

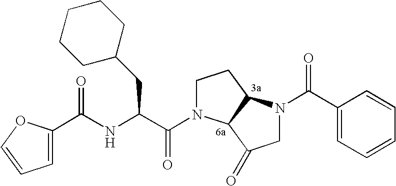

HPLC Rt=16.66 mins (>90%), HPLC-MS 478.1 [M+H]⁺, 977.3 [2M+Na]⁺.

### EXAMPLE 289

### (3aR,6aS)-Furan-3-carboxylic acid [(1S)-2-(4-benzoyl-6-oxo-hexahydro-pyrrolo[3,2-b]pyrrole-1-yl)-1-cyclohexylmethyl-2-oxo-ethyl]-amide

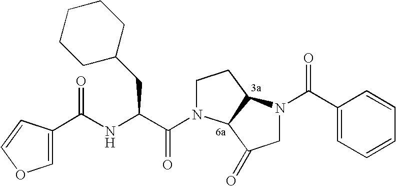

HPLC Rt=16.37 mins (>90%), HPLC-MS 478.1 [M+H]⁺, 977.3 [2M+Na]⁺.

### EXAMPLE 290

### (3aR,6aS)-N-[(1S)-2-(4-Benzoyl-6-oxo-hexahydro-pyrrolo[3,2-b]pyrrol-1-yl)-1-cyclohexylmethyl-2-oxo-ethyl]-benzamide

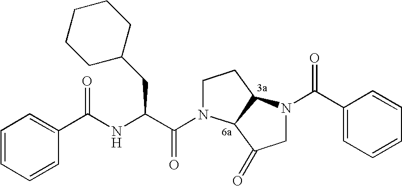

HPLC Rt=16.71 mins (>95%), HPLC-MS 488.2 [M+H]⁺, 997.3 [2M+Na]⁺.

### EXAMPLE 291

### (3aR,6aS)-Thiophene-3-carboxylic acid {(1S)-1-cyclohexylmethyl-2-[4-(2-methyl-benzoyl)-6-oxo-hexahydro-pyrrolo[3,2-b]pyrrol-1-yl]-2-oxo-ethyl}-amide

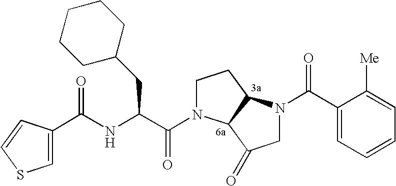

HPLC Rt=15.65 mins (>95%), HPLC-MS 508.2 [M+H]⁺, 526.2 [M+H+H₂O]⁺.

### EXAMPLE 292

### (3aR,6aS)-Thiophene-3-carboxylic acid {(1S)-1-cyclohexylmethyl-2-[4-(3-methyl-benzoyl)-6-oxo-hexahydro-pyrrolo[3,2-b]pyrrol-1-yl]-2-oxo-ethyl}-amide

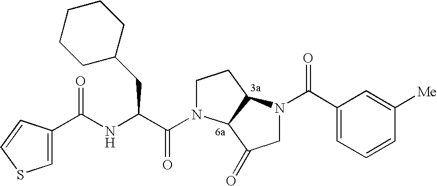

HPLC Rt=16.10 mins (>90%), HPLC-MS 508.1 [M+H]⁺.

### EXAMPLE 293

### (3aR,6aS)-Thiophene-3-carboxylic acid {(1S)-1-cyclohexylmethyl-2-[4-(4-methyl-benzoyl)-6-oxo-hexahydro-pyrrolo[3,2-b]pyrrol-1-yl]-2-oxo-ethyl}-amide

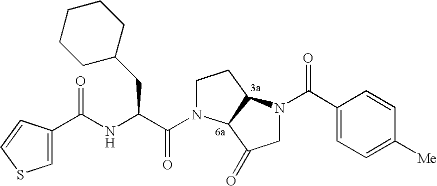

HPLC Rt=15.88 mins (>90%), HPLC-MS 508.1 [M+H]⁺.

### EXAMPLE 294

### (3aR,6aS)-Thiophene-3-carboxylic acid [(1R)-2-(4-benzoyl-6-oxo-hexahydro-pyrrolo[3,2-b]pyrrole-1-yl)-1-methanesulfonylmethyl-2-oxo-ethyl]-amide

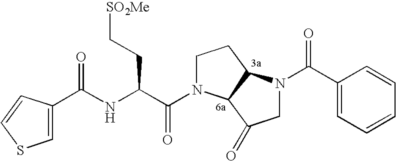

HPLC Rt=8.61 mins (>90%), HPLC-MS 504.1 [M+H]⁺.

### EXAMPLE 295

### (3aR,6aS)-Furan-2-carboxylic acid {(1S)-2-[4-((2S)-2-acetylamino-4-methyl-pentanoyl)-6-oxo-hexahydro-pyrrolo[3,2-b]pyrrole-1-yl]-1-cyclohexylmethyl-2-oxo-ethyl}-amide

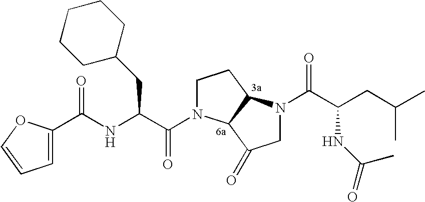

HPLC Rt=14.35 mins (>95%), HPLC-MS 529.2 [M+H]⁺.

In addition, EXAMPLES 7, 8, 9, 10, 12, 13, 14, 16, 17, 19, 20, 37, 39, 59, 61, 65, 85, 86, 87, 88, 89, 90, 91, 92, 93, 98, 103, 123, 145, 151, 154, 158, 159, 161, 164, 170, 171, 172, 173, 174, 185, 187, 193, 194, 245, 247, 249a, 249b, 249c, 298, 303, 310, 314, 315, 316, 317, 323, 329, 330, 334, 335 and 340 have utility as inhibitors of cathepsin S with Ki less than 5000 nM.

EXAMPLES 296 to 345 were prepared as detailed for EXAMPLES 1 and 119, substituting the appropriate carboxylic acids as required and are inhibitors of cathepsin L with Ki ranging from 10-5000 nM;

### EXAMPLE 296

### (3aR,6aS)-Naphthalene-1-carboxylic acid [(1S)-2-(4-benzoyl-6-oxo-hexahydro-pyrrolo[3,2-b]pyrrole-1-yl)-1-(4-hydroxybenzyl)-2-oxo-ethyl]-amide

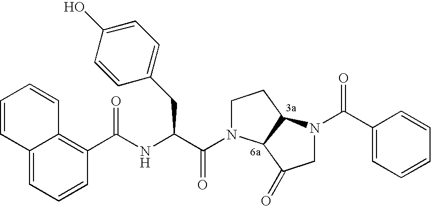

HPLC Rt=13.5-14.4 mins (>90%), HPLC-MS 548.2 [M+H]⁺, 1117.4 [2M+Na]⁺.

### EXAMPLE 297

### (3aR,6aS)-Quinoline-8-carboxylic acid [(1S)-2-(4-benzoyl-6-oxo-hexahydro-pyrrolo[3,2-b]pyrrole-1-yl)-1-(4-hydroxy-benzyl)-2-oxo-ethyl]-amide

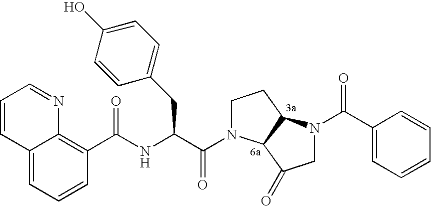

HPLC Rt=11.79 mins (>85%), HPLC-MS 549.2 [M+H]⁺, 567.3 [M+H+H₂O]⁺.

### EXAMPLE 298

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-3-bromo-benzamide

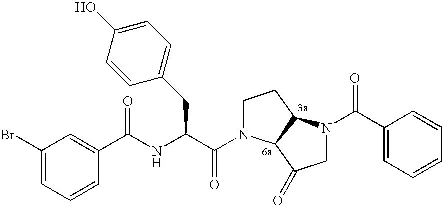

HPLC Rt=13.21 mins (>95%), HPLC-MS 576.1/578.1 [M+H]⁺.

### EXAMPLE 299

### (3aR,6aS)-Isoquinoline-1-carboxylic acid [(1S)-2-(4-benzoyl-6-oxo-hexahydro-pyrrolo[3,2-b]pyrrol-1-yl)-1-(4-hydroxy-benzyl)-2-oxo-ethyl]-amide

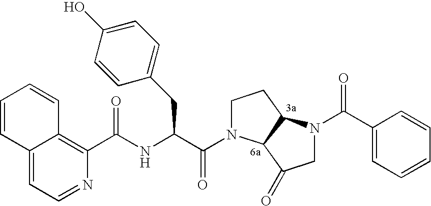

HPLC Rt=14.1-15.1 mins (>85%), HPLC-MS 549.2 [M+H]⁺, 567.2 [M+H+H₂O]⁺.

### EXAMPLE 300

### (3aR,6aS)-Benzo[b]thiophene-2-carboxylic acid [(1S)-2-(4-benzoyl-6-oxo-hexahydro-pyrrolo[3,2-b]pyrrole-1-yl)-1-(4-hydroxy-benzyl)-2-oxo-ethyl]-amide

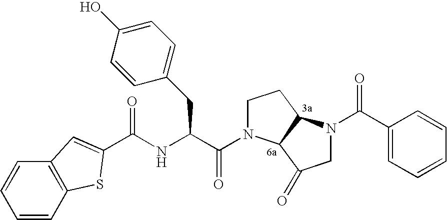

HPLC Rt=14.8-15.6 mins (>85%), HPLC-MS 554.2 [M+H]⁺.

### EXAMPLE 301

### (3aR,6aS)-Naphthalene-1-carboxylic acid {(1S)-1-(4-hydroxy-benzyl)-2-[4-(naphthalene-1-carbonyl)-6-oxo-hexahydro-pyrrolo[3,2-b]pyrrol-1-yl]-2-oxo-ethyl}-amide

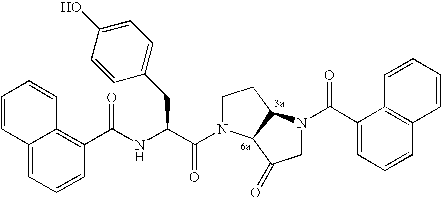

HPLC Rt=15.5-16.8 mins (>85%), HPLC-MS 598.2 [M+H]⁺, 616.2 [M+H+H₂O]⁺.

### EXAMPLE 302

### (3aR,6aS)-Naphthalene-1-carboxylic acid {(1S)-1-(4-hydroxy-benzyl)-2-[4-(naphthalene-2-carbonyl)-6-oxo-hexahydro-pyrrolo[3,2-b]pyrrol-1-yl]-2-oxo-ethyl}-amide

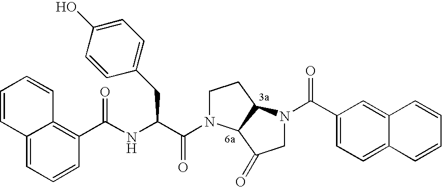

HPLC Rt=16.0-16.9 mins (>90%), HPLC-MS 598.2 [M+H]⁺.

### EXAMPLE 303

### (3aR,6aS)-3-Bromo-N-{(1S)-1-(4-hydroxy-benzyl)-2-[4-(2-methane sulfonyl-benzoyl)-6-oxo-hexahydro-pyrrolo[3,2-b]pyrrole-1-yl]-2-oxo-ethyl}-benzamide

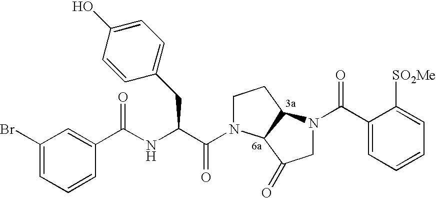

HPLC Rt=12.66 mins (>90%), HPLC-MS 654.0/656.0 [M+H]⁺.

### EXAMPLE 304

### (3aR,6aS)-3-Bromo-N-{(1S)-1-(4-hydroxy-benzyl)-2-[4-(3-methane sulfonyl-benzoyl)-6-oxo-hexahydro-pyrrolo[3,2-b]pyrrole-1-yl]-2-oxo-ethyl}-benzamide

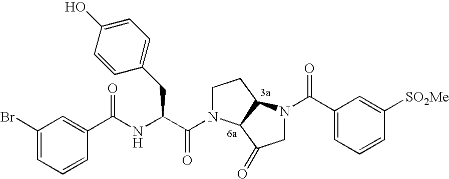

HPLC Rt=12.95 mins (>75%), HPLC-MS 654.0/656.0 [M+H]⁺.

### EXAMPLE 305

### (3aR,6aS)-3-Bromo-N-{(1S)-1-(4-hydroxy-benzyl)-2-[4-(3-nitro-benzoyl)-6-oxo-hexahydro-pyrrolo[3,2-b]pyrrole-1-yl]-2-oxo-ethyl}-benzamide

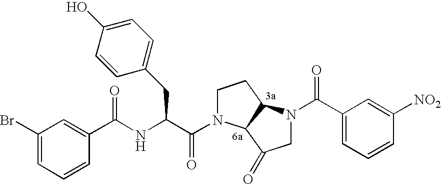

HPLC Rt=13.52 mins (>90%), HPLC-MS 621.0/623.0 [M+H]⁺.

### EXAMPLE 306

### (3aR,6aS)-3-Bromo-N-{(1S)-1-(4-hydroxy-benzyl)-2-[4 (4-nitro-benzoyl)-6-oxo-hexahydro-pyrrolo[3,2-b]pyrrole-1-yl]-2-oxo-ethyl)-benzamide

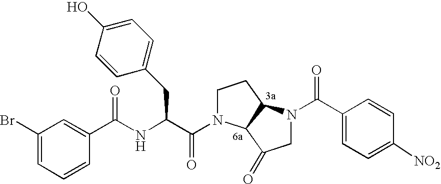

HPLC Rt=13.50 mins (>95%), HPLC-MS 621.0/623.0 [M+H]⁺.

### EXAMPLE 307

### (3aR,6aS)-N-[(1S)-2-[4-(Adamantane-1-carbonyl)-6-oxo-hexa hydro-pyrrolo[3,2-b]pyrrole-1-yl)-1-(4-hydroxy-benzyl)-2-oxo-ethyl]-3-bromo-benzamide

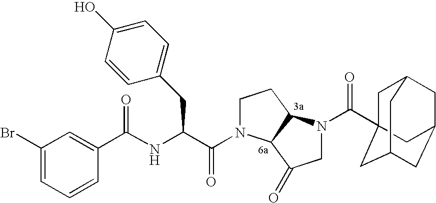

HPLC Rt=15.9-17.3 mins (>50%), HPLC-MS 634.0/636.0 [M+H]⁺.

### EXAMPLE 308

### (3aR,6aS)-3-Bromo-N-[(1S)-2-[4-(hexahydro-2,5-methano-pentalene-3a-carbonyl)-6-oxo-hexahydro-pyrrolo[3,2-b]pyrrole-1-yl]-1-(4-hydroxy-benzyl)-2-oxo-ethyl]-benzamide

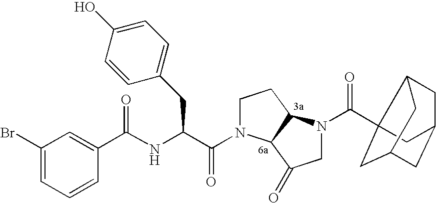

HPLC Rt=14.7-16.2 mins (>80%), HPLC-MS 620.0/622.0 [M+H]⁺, 638.1/640.1 [M+H+H₂O]⁺.

### EXAMPLE 309

### (3aR,6aS)-3-Bromo-N-[(1S)-2-[4-(4-hydroxy-benzoyl)-6-oxo-hexa hydro-pyrrolo[3,2-b]pyrrole-1-yl]-1-(4-hydroxy-benzyl)-2-oxo-ethyl]-benzamide

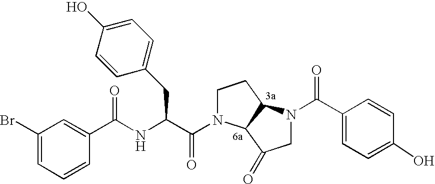

HPLC Rt=11.92 mins (>80%), HPLC-MS 592.0/594.0 [M+H]⁺.

### EXAMPLE 310

### (3aR,6aS)-N-[(1S)-2-[4-(4-Amino-benzoyl)-6-oxo-hexahydro-pyrrolo[3,2-b]pyrrole-1-yl]-1-(4-hydroxy-benzyl)-2-oxo-ethyl]-3-bromo-benzamide

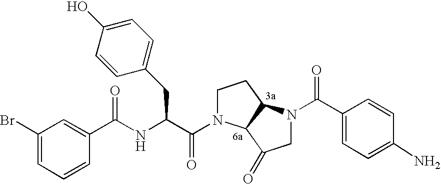

HPLC Rt=9.9-10.6 mins (>75%), HPLC-MS 591.0/593.0 [M+H]⁺, 609.0/611.0 [M+H+H₂O]⁺.

### EXAMPLE 311

### (3aR,6aS)-3-Bromo-N-{(1S)-1-(4-hydroxy-benzyl)-2-oxo-2-[6-oxo-4-(quinoline-6-carbonyl)-hexahydro-pyrrolo[3,2-b]pyrrol-1-yl]-ethyl}-benzamide

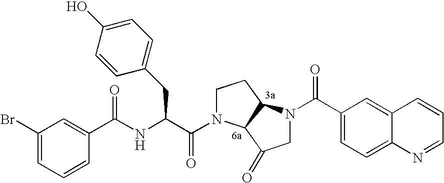

HPLC Rt=11.13 mins (>75%), HPLC-MS 627.0/629.0 [M+H]⁺, 645.0/647.0 [M+H+H₂O]⁺.

### EXAMPLE 312

### (3aR,6aS)-4,5-Dimethyl-furan-2-carboxylic acid {(1S)-1-(4-hydroxy-benzyl)-2-[4-(2-methanesulfonyl-benzoyl)-6-oxo-hexahydro-pyrrolo[3,2-b]pyrrol-1-yl]-2-oxo-ethyl}-amide

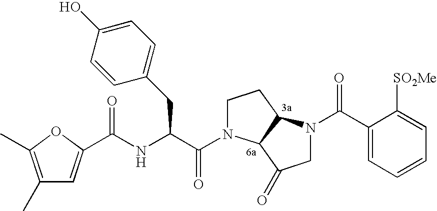

HPLC Rt=11.43 mins (>90%), HPLC-MS 594.1 [M+H]⁺.

### EXAMPLE 313

### (3aR,6aS)-2-Ethyl-5-methyl-2H-pyrazole-3-carboxylic acid {(1S)-1-(4-hydroxy-benzyl)-2-[4-(2-methanesulfonylbenzoyl)-6-oxo-hexahydropyrrolo[3,2-b]pyrrole-1-yl]-2-oxo-ethyl}-amide

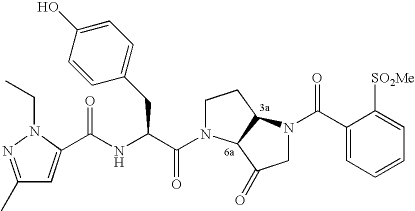

HPLC Rt=10.03 mins (>90%), HPLC-MS 608.1 [M+H]⁺, 626.1 [M+H+H₂O]⁺.

### EXAMPLE 314

### (3aR,6aS)-N-[(1S)-2-[Benzo[b]thiophene-3-carbonyl)-6-oxo-hexahydro-pyrrolo[3,2-b]pyrrole-1-yl]-1-(3-methyl-benzyl)-2-oxo-ethyl]-2,2-diphenyl-acetamide

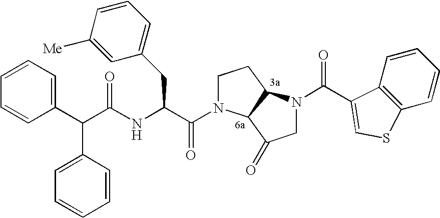

HPLC Rt=20.2-21.4 mins (>95%), HPLC-MS 642.2 [M+H]⁺.

### EXAMPLE 315

### (3aR,6aS)-N-[(1S)-2-[4-(Benzo[b]thiophene-3-carbonyl)-6-oxo-hexahydro-pyrrolo[3,2-b]pyrrole-1-yl]-1-(3-fluoro-benzyl)-2-oxo-ethyl]-2,2-diphenyl-acetamide

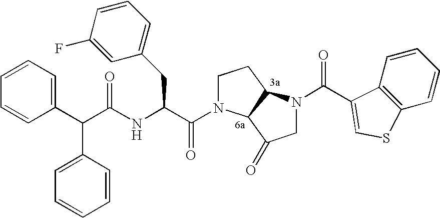

HPLC Rt=19.7-21.0 mins (>90%), HPLC-MS 646.2 [M+H]⁺.

### EXAMPLE 316

### (3aR,6aS)-N-[(1S)-2-[4-(Benzo[b]thiophene-3-carbonyl)-6-oxo-hexahydro-pyrrolo[3,2-b]pyrrole-1-yl]-1-(3,5-difluoro-benzyl)-2-oxo-ethyl]-2,2-diphenyl-acetamide

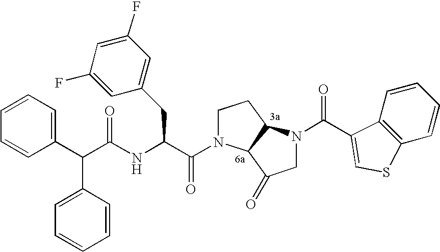

HPLC Rt=20.1-21.3 mins (>90%), HPLC-MS 664.2 [M+H]⁺.

### EXAMPLE 317

### (3aR,6aS)-Benzothiazole-6-carboxylic acid [(1S)-2-(4-benzoyl-6-oxo-hexahydro-pyrrolo[3,2-b]pyrrole-1-yl)-1-(4-hydroxy-benzyl)-2-oxo-ethyl]-amide

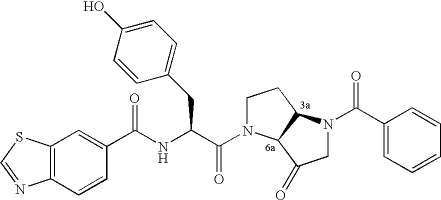

HPLC Rt=12.02 mins (>95%), HPLC-MS 555.1 [M+H]⁺.

### EXAMPLE 318

### (3aR,6aS)-4-Aminomethyl-N-[(1S)-2-(4-benzoyl-6-oxo-hexahydro-pyrrolo[3,2-b]pyrrol-1-yl)-1-(hydroxy-benzyl)-2-oxo-ethyl]-benzamide

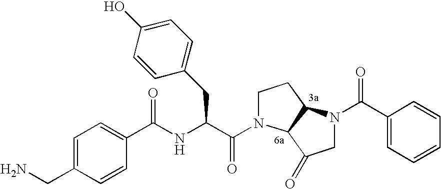

HPLC Rt=8.92 mins (>95%), HPLC-MS 527.2 [M+H]⁺, 545.2 [M+H+H₂O]⁺.

### EXAMPLE 319

### (3aR,6aS)-3-Aminomethyl-N-[(1S)-2-(4-benzoyl-6-oxo-hexahydro-pyrrolo[3,2-b]pyrrole-1-yl)-1-(4-hydroxy-benzyl)-2-oxo-ethyl]-benzamide

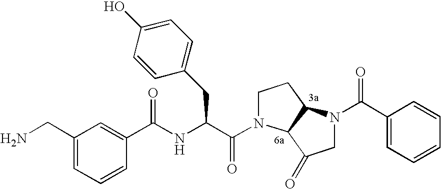

HPLC Rt=9.14 mins (>°/0), HPLC-MS 527.2 [M+H]⁺, 545.3 [M+H+H₂O]⁺.

### EXAMPLE 320

### (3aR,6aS)-N-[(1S)-2-(4-Benzenesulfonyl-6-oxo-hexahydro-pyrrolo[3,2-b]pyrrol-1-yl)-1-(4-hydroxy-benzyl)-2-oxo-ethyl]-3-bromo-benzamide

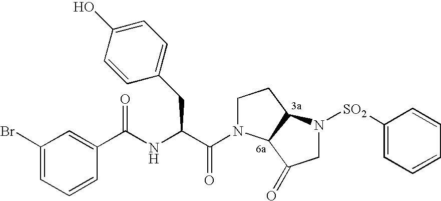

HPLC Rt=16.1-17.9 mins (>90%), HPLC-MS 612.0/614.0 [M+H]⁺.

### EXAMPLE 321

### (3aR,6aS)-4-Dimethylamino-N-{(1S)-1-(4-hydroxy-benzyl)-2-[4-(3-nitro-benzoyl)-6-oxo-hexahydro-pyrrolo[3,2-b]pyrrole-1-yl]-2-oxo-ethyl}-benzamide

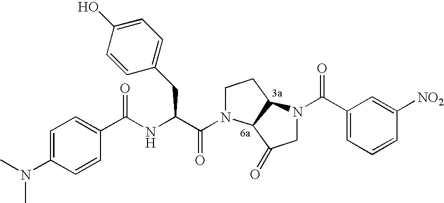

HPLC Rt=10.75 mins (>80%), HPLC-MS 586.2 [M+H]⁺, 604.2 [M+H+H₂O]⁺.

### EXAMPLE 322

### (3aR,6aS)-4-Dimethylamino-N-[(1S)-2-[((3-fluoro-benzoyl)-6-oxo-hexahydro-pyrrolo[3,2-b]pyrrole-1-yl]-1-(4-hydroxy-benzyl)-2-oxo-ethyl]-benzamide

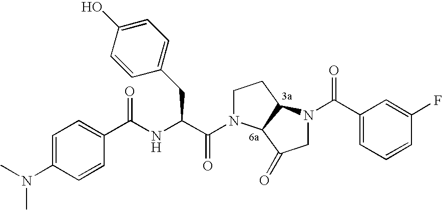

HPLC Rt=10.59 mins (>90%), HPLC-MS 559.2 [M+H]⁺, 577.2 [M+H+H₂O]⁺.

### EXAMPLE 323

### (3aR,6aS)-N-[(1S)-2-[4-(Benzo[b]thiophene-3-carbonyl)-6-oxo-hexahydro-pyrrolo[3,2-b]pyrrole-1-yl]-1-(4-hydroxy-benzyl)-2-oxo-ethyl]-3-bromo-benzamide

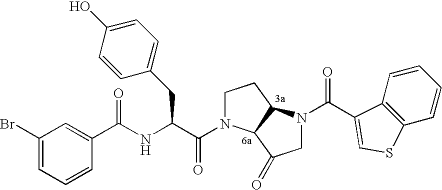

HPLC Rt=14.61 mins (>90%), HPLC-MS 632.0/634.0 (M+H]⁺.

### EXAMPLE 324

### (3aR,6aS)-3-Bromo-N-{(1S)-1-(4-hydroxy-benzyl)-2-oxo-2-[6-oxo-4-(thiophene-2-carbonyl)-hexahydro-pyrrolo[3,2-b]pyrrol-1-yl]-ethyl}-benzamide

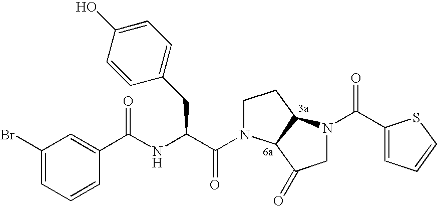

HPLC Rt=12.93 mins (>90%), HPLC-MS 582.0/584.0 [M+H]⁺.

### EXAMPLE 325

### (3aR,6aS)-3-Bromo-N-[(1S)-2-(4-cyclohexanecarbonyl-6-oxo-hexahydro-pyrrolo[3,2-b]pyrrole-1-yl)-1-(4-hydroxy-benzyl)-2-oxo-ethyl]-benzamide

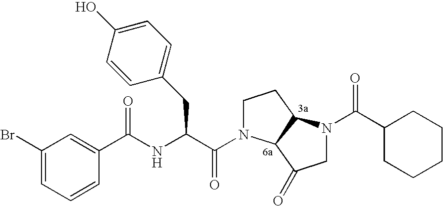

HPLC Rt=15.0-17.0 mins (>90%), HPLC-MS 582.1/584.1 [M+H]⁺.

### EXAMPLE 326

### (3aR,6aS)-3-Bromo-N-{(1S)-1-(4-hydroxy-benzyl)-2-[4-(2-methyl-benzoyl)-6-oxo-hexahydro-pyrrolo[3,2-b]pyrrole-1-yl]-2-oxo-ethyl}-benzamide

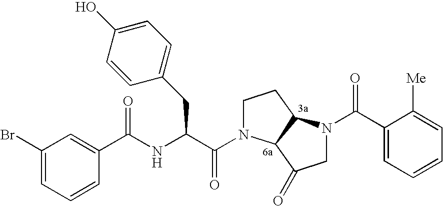

HPLC Rt=14.4-16.1 mins (>90%), HPLC-MS 590.0/592.0 [M+H]⁺.

### EXAMPLE 327

### (3aR,6aS)-3-Bromo-N-{(1S)-1-(4-hydroxy-benzyl)-2-[4-(3-methyl-benzoyl)-6-oxo-hexahydro-pyrrolo[3,2-b]pyrrol-1-yl]-2-oxo-ethyl}-benzamide

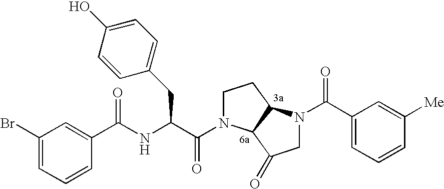

HPLC Rt=14.4-15.8 mins (>90%), HPLC-MS 590.0/592.0 [M+H]⁺.

### EXAMPLE 328

### (3aR,6aS)-3-Bromo-N-{(1S)-1-(4-hydroxy-benzyl)-2-[4-(4-methyl-benzoyl)-6-oxo-hexahydro-pyrrolo[3,2-b]pyrrole-1-yl]-2-oxo-ethyl}-benzamide

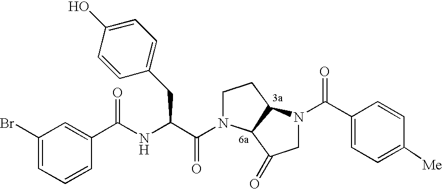

HPLC Rt=13.8-14.9 mins (>90%), HPLC-MS 590.0/592.0 [M+H]⁺.

### EXAMPLE 329

### (3aR,6aS)-N-[(1S)-2-[4-(Benzo[b]thiophene-3-carbonyl)-6-oxo-hexahydro-pyrrolo[3,2-b]pyrrole-1-yl]-1-(4-hydroxy-benzyl)-2-oxo-ethyl]-2,2-diphenyl-acetamide

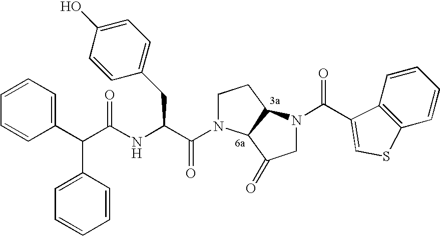

HPLC Rt=17.0-18.5 mins (>80%), HPLC-MS 644.1 [M+H]⁺.

### EXAMPLE 330

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-2,2-diphenyl-acetamide

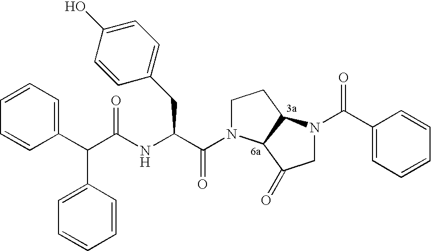

HPLC Rt=16.4-17.5 mins (>85%), HPLC-MS 588.2 [M+H]⁺.

### EXAMPLE 331

### (3aR,6aS)-3-Aminomethyl-N-[(1S)-2-[4-(benzo[b]thiophene-3-carbonyl)-6-oxo-hexahydro-pyrrolo[3,2-b]pyrrole-1-yl]-1-(4-hydroxy-benzyl)-2-oxo-ethyl]-benzamide

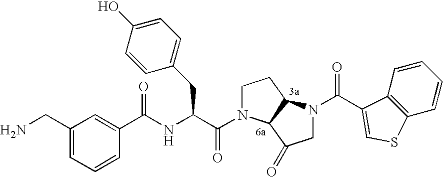

HPLC Rt=10.82 mins (>90%), HPLC-MS 583.1 [M+H]⁺.

### EXAMPLE 332

### (3aR,6aS)-3-Aminomethyl-N-{(1S)-1-(4-hydroxy-benzyl)-2-[4-(2-methanesulfonyl-benzoyl)-6-oxo-hexahydro-pyrrolo[3,2-b]pyrrole-1-yl]-2-oxo-ethyl}-benzamide

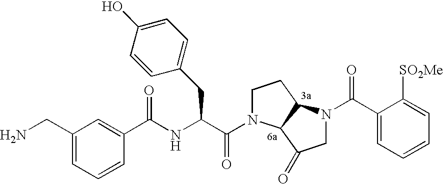

HPLC Rt=8.16 mins (>85%), HPLC-MS 605.1 [M+H]⁺, 623.2 [M+H+H₂O]⁺.

### EXAMPLE 333

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-benzamide

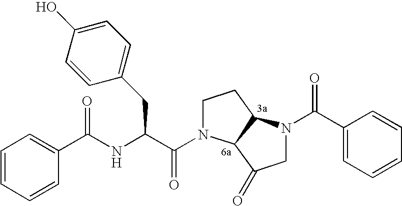

HPLC Rt=11.79 mins (>95%), HPLC-MS 498.1 [M+H]⁺.

### EXAMPLE 334

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-3-chloro-benzamide

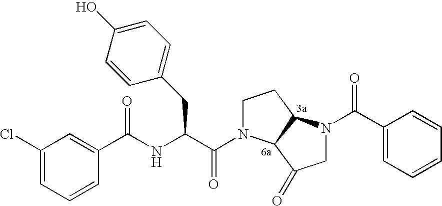

HPLC Rt=13.64 mins (>95%), HPLC-MS 532.1/534.1 [M+H]⁺.

### EXAMPLE 335

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-3-fluoro-benzamide

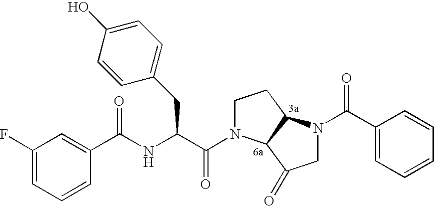

HPLC Rt=12.10 mins (>90%), HPLC-MS 516.1 [M+H]⁺.

### EXAMPLE 336

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-nicotinamide

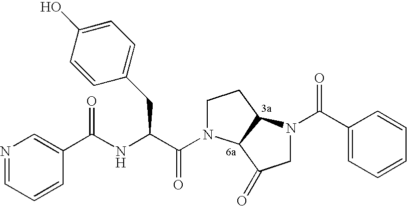

HPLC Rt=8.16 mins (>95%), HPLC-MS 499.1 [M+H]⁺, 997.2 [2M+H]⁺.

### EXAMPLE 337

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl)-3-bromo-4-methyl-benzamide

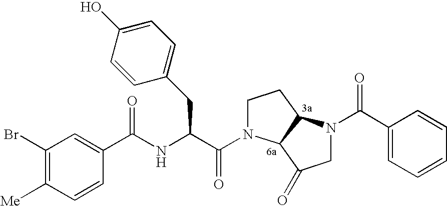

HPLC Rt=14.0-15.1 mins (>85%), HPLC-MS 590.0/592.0 [M+H]⁺.

### EXAMPLE 338

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-3-methoxy-benzamide

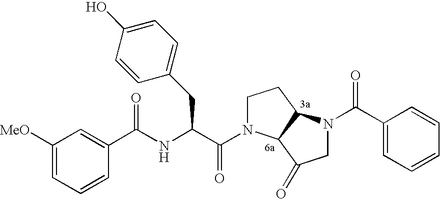

HPLC Rt=11.81 mins (>95%), HPLC-MS 528.1 [M+H]⁺.

### EXAMPLE 339

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl]-3-trifluoromethoxy-benzamide

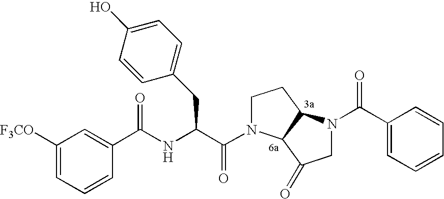

HPLC Rt=14.21 mins (>90%), HPLC-MS 582.1 [M+H]⁺, 600.2 [M+H+H₂O]⁺.

### EXAMPLE 340

### (3aR,6aS)-N-[(1S)-1-(4-Benzoyl-6-oxo-hexahydro-pyrrolo[3,2-b]pyrrole-1-carbonyl)-3-methyl-butyl]-3-bromo-benzamide

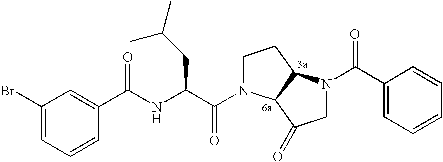

HPLC Rt=15.0-16.1 mins (>85%), HPLC-MS 526.1/528.1 [M+H]⁺.

### EXAMPLE 341

### (3aR,6aS)-N-[(1S)-2-(4-Benzoyl-6-oxo-hexahydro-pyrrolo[3,2-b]pyrrol-1-yl)-1-(3-methyl-benzyl)-2-oxo-ethyl]-3-bromo-benzamide

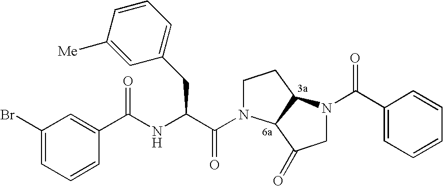

HPLC Rt=17.0-18.3 mins (>85%), HPLC-MS 574.0/576.0[M+H]⁺.

### EXAMPLE 342

### (3aR,6aS)-N-[(1S)-2-(4-Benzoyl-6-oxo-hexahydro-pyrrolo[3,2-b]pyrrol-1-yl)-1-(3-fluoro-benzyl)-2-oxo-ethyl]-3-bromo-benzamide

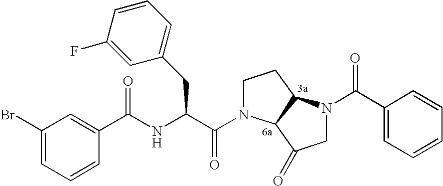

HPLC Rt=15.8-17.1 mins (>90%), HPLC-MS 578.0/580.0 [M+H]⁺.

### EXAMPLE 343

### (3aR,6aS)-N-[(1S)-2-(4-Benzoyl-6-oxo-hexahydro-pyrrolo[3,2-b]pyrrol-1-yl)-1-(3,5-difluoro-benzyl)-2-oxo-ethyl]-3-bromo-benzamide

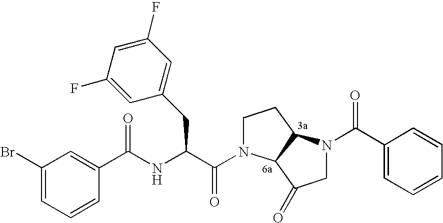

HPLC Rt=15.9-17.3 mins (>90%), HPLC-MS 596.0/598.0 (M+H]⁺.

### EXAMPLE 344

### (3aR,6aS)-N-[(1S)-2-(4-Benzoyl-6-oxo-hexahydro-pyrrolo[3,2-b] pyrrol-1-yl)-1-(4-fluoro-benzyl)-2-oxo-ethyl]-3-bromo-benzamide

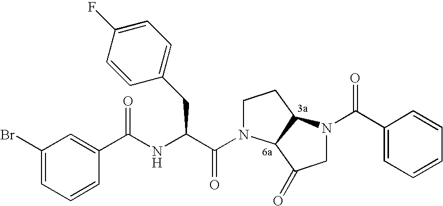

HPLC Rt=17.1-18.2 mins (>90%), HPLC-MS 578.0/580.0 [M+H]⁺.

### EXAMPLE 345

### (3aR,6aS)-N-[(1S)-2-[4-((2S)-2-Acetylamino-4-methyl-pentanoyl)-6-oxo-hexahydro-pyrrolo[3,2-b]pyrrol-1-yl]-1-(4-hydroxy-benzyl)-2-oxo-ethyl]-3-bromo-benzamide

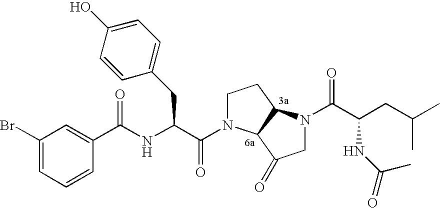

HPLC Rt=12.73 mins (>90%), HPLC-MS 627.0/629.0 [M+H]⁺.

In addition, EXAMPLES 7, 8, 9, 10, 12, 14, 15, 16, 17, 18, 28, 31, 37, 59, 63, 65, 68, 73, 85, 86, 87, 88, 89, 90, 91, 92, 93, 98, 103, 104, 111, 113, 117, 118, 145, 151, 154, 158, 159, 161, 164, 170, 171, 172, 173, 174, 175, 178, 179, 180, 181, 182, 185, 193, 194, 204, 216, 244, 245, 246, 247, 249a, 249b, 249c, 250, 251, 254, 255, 256, 258, 259, 261, 262, 268, 269, 270, 271, 272, 273, 275, 278, 280, 281, 282, 285, 286, 287, 288, 289, 290, 291, 292, 293, 295, 346, 356, 357, 358 and 359 have utility as inhibitors of cathepsin L with Ki less than 5000 nM.

EXAMPLES 346 to 359 were prepared as detailed for EXAMPLES 1 and 119, substituting the appropriate carboxylic acids as required and are inhibitors of cruzain and cruzipains with Ki ranging from 10-5000 nM;

### EXAMPLE 346

### (3aR,6aS)-N-[(1S)-2-(4-Benzoyl-6-oxo-hexahydro-pyrrolo[3,2-b]pyrrol-1-yl)-1-(4-hydroxybenzyl)-2-oxo-ethyl]-4-tert-butyl-benzamide

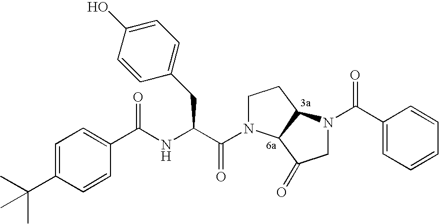

HPLC Rt=16.0-17.1 mins (>90%), HPLC-MS 554.3 [M+H]⁺, 1129.5 [2M+Na]⁺.

### EXAMPLE 347

### (3aR,6aS)-4-tert-Butyl-N-{(1-(4-fluoro-benzyl)-2-oxo-2-[6-oxo-4-(pyridin-3-carbonyl)-hexahydro-pyrrolo[3,2-b]pyrrole-1-yl]-ethyl}-benzamide

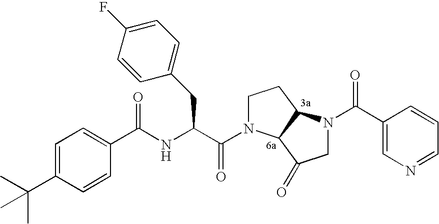

HPLC Rt=16.82 mins (>90%), HPLC-MS 557.2 [M+H]⁺.

### EXAMPLE 348

### (3aR,6aS)-4-tert-Butyl-N-{(1S)-1-(4-fluoro-benzyl)-2-oxo-2-[6-oxo-4-(pyridine-4-carbonyl)-hexahydro-pyrrolo[3,2-b]pyrrole-1-yl]-ethyl}-benzamide

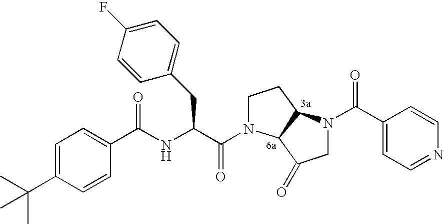

HPLC Rt=16.44 mins (>85%), HPLC-MS 557.2 [M+H]⁺, 575.3 [M+H+H₂O]⁺.

### EXAMPLE 349

### (3aR,6aS)-4-tert-Butyl-N-{(1S)-1-(4-fluoro-benzyl)-2-[4-(naphthalene-1-carbonyl)-6-oxo-hexahydro-pyrrolo[3,2-b]pyrrole-1-yl]-2-oxo-ethyl}-benzamide

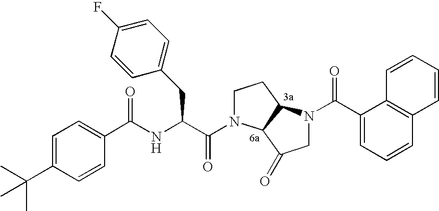

HPLC Rt=19.6-20.8 mins (>80%), HPLC-MS 606.1 [M+H]⁺.

### EXAMPLE 350

### (3aR,6aS)-4-tert-Butyl-N-{(1S)-1-(4-fluoro-benzyl)-2-[4-(naphthalene-2-carbonyl)-6-oxo-hexahydro-pyrrolo[3,2-b]pyrrole-1-yl]-2-oxo-ethyl}-benzamide

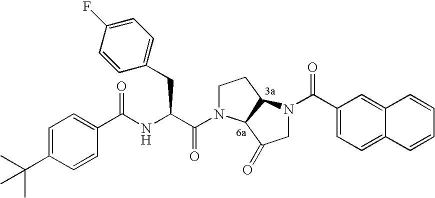

HPLC Rt=21.0-22.0 mins (>85%), HPLC-MS 606.1 [M+H]⁺.

### EXAMPLE 351

### (3aR,6aS)-4-tert-Butyl-N-{(1S)-1-(4-hydroxy-benzyl)-2-oxo-2-[6-oxo-4-(pyridine-3-carbonyl)-hexahydro-pyrrolo[3,2-b]pyrrole-1-yl]-ethyl}-benzamide

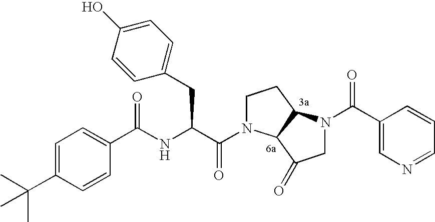

HPLC Rt=14.27 mins (>85%), HPLC-MS 555.3 [M+H]⁺, 573.3 [M+H+H₂O]⁺.

### EXAMPLE 352

### (3aR,6aS)-4-tert-Butyl-N-{(1S)-1-(4-hydroxy-benzyl)-2-oxo-2-[6-oxo-4-(pyridine-4-carbonyl)-hexahydro-pyrrolo[3,2-b]pyrrole-1-yl]-ethyl}-benzamide

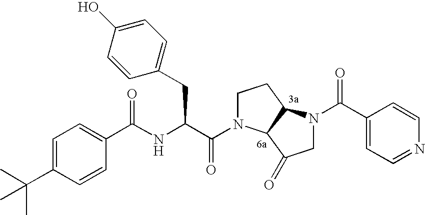

HPLC Rt=14.35 mins (>90%), HPLC-MS 555.2 [M+H]⁺, 573.3 [M+H+H₂O]⁺.

### EXAMPLE 353

### (3aR,6aS)-4-tert-Butyl-N-{(1S)-1-(4-hydroxy-benzyl)-2-[4-(naphthalene-1-carbonyl)-6-oxo-hexahydro-pyrrolo[3,2-b]pyrrole-1-yl]-2-oxo-ethyl}-benzamide

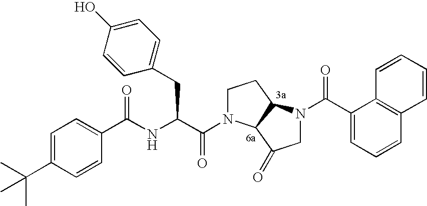

HPLC Rt=17.8-18.7 mins (>85%), HPLC-MS 604.2 [M+H]⁺.

### EXAMPLE 354

### (3aR,6aS)-4-tert-Butyl-N-{(1S)-1-(4-hydroxy-benzyl)-2-[4-(naphthalene-2-carbonyl)-6-oxo-hexahydro-pyrrolo[3,2-b]pyrrole-1-yl]-2-oxo-ethyl}-benzamide

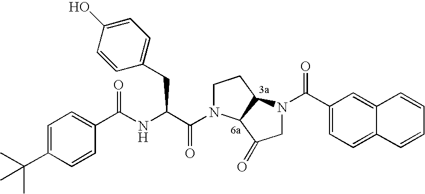

HPLC Rt=17.3-18.2 mins (>85%), HPLC-MS 604.1 [M+H]⁺.

### EXAMPLE 355

### (3aR,6aS)-Quinoline-4-carboxylic acid [(1S)-2-(4-benzoyl-6-oxo-hexahydro-pyrrolo[3,2-b]pyrrole-1-yl)-1-(4-hydroxy-benzyl)-2-oxo-ethyl]-amide

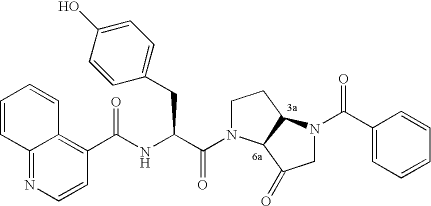

HPLC Rt=10.10 mins (>90%), HPLC-MS 549.2 [M+H]⁺, 567.2 [M+H+H₂O]⁺.

### EXAMPLE 356

### (3aR,6aS)-N-[(1S)-2-(4-Benzoyl-6-oxo-hexahydro-pyrrolo[3,2-b]pyrrol-1-yl)-1-(4-hydroxy-benzyl)-2-oxo-ethyl)-4-dimethylamino-benzamide

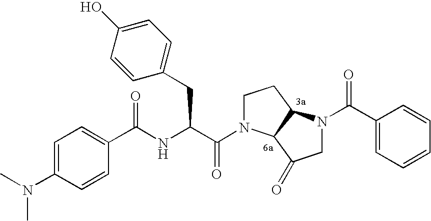

HPLC Rt=10.34 mins (>90%), HPLC-MS 541.2 [M+H]⁺.

### EXAMPLE 357

### (3aR,6aS)-4-Dimethylamino-N-{(1S)-1-(4-hydroxy-benzyl)-2-[4-(2-methanesulfonyl-benzoyl)-6-oxo-hexahydro-pyrrolo[3,2-b]pyrrole-1-yl]-2-oxo-ethyl}-benzamide

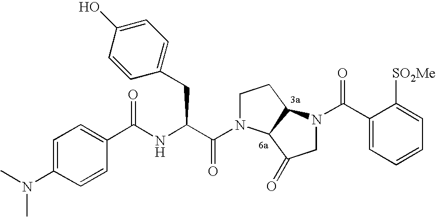

HPLC Rt=9.78 mins (>90%), HPLC-MS 619.2 [M+H]⁺, 637.2 [M+H+H₂O]⁺.

### EXAMPLE 358

### (3aR,6aS)-4-Dimethylamino-N-{(1S)-1-(4-hydroxy-benzyl)-2-[4-(3-methanesulfonyl-benzoyl)-6-oxo-hexahydro-pyrrolo[3,2-b]pyrrol-1-yl]-2-oxo-ethyl}-benzamide

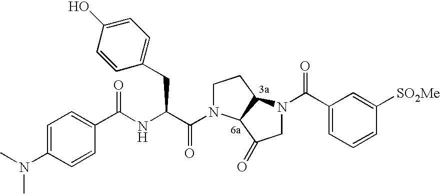

HPLC Rt=9.95 mins (>80%), HPLC-MS 619.2 [M+H]⁺, 637.2 [M+H+H₂O]⁺.

### EXAMPLE 359

### (3aR,6aS)-4-Dimethylamino-N-{(1S)-1-(4-hydroxy-benzyl)-2-oxo-2-[6-oxo-4-(thiophene-2-carbonyl)-hexahydro-pyrrolo[3,2-b]pyrrol-1-yl]-ethyl}-benzamide

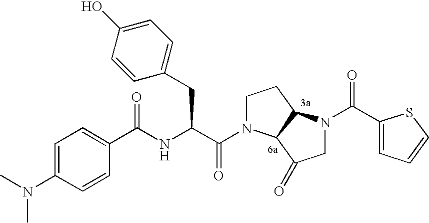

HPLC Rt=10.69 mins (>85%), HPLC-MS 547.2 [M+H]⁺, 565.2 (M+H+ H₂O]⁺.

In addition, EXAMPLES 1, 2, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 39, 42, 43, 44, 46, 47, 48, 49, 50, 51, 53, 54, 55, 56, 58, 59, 60, 61, 62, 63, 64, 65, 66, 68, 69, 70, 71, 72, 73, 74, 77, 79, 80, 81, 82, 85, 86, 87, 88, 89, 90, 91, 92, 93, 94, 95, 96, 97, 98, 99, 100, 101, 102, 103, 104, 105, 111, 113, 114, 115, 116, 117, 118, 128, 130, 132, 133, 134, 135, 136, 137, 138, 139, 140, 141, 142, 145, 147, 150, 151, 154, 156, 157, 158, 159, 160, 161, 162, 164, 165, 166, 167, 168, 170, 171, 172, 173, 174, 175, 180, 183, 184, 185, 190, 191, 192, 193, 194, 196, 197, 199, 200, 203, 204, 205, 206, 207, 208, 209, 210, 212, 214, 216, 217, 218, 220, 221, 223, 224, 226, 227, 228, 233, 235, 238, 240, 241, 242, 244, 245, 246, 247, 248, 249a, 249b, 249c, 254, 255, 256, 260, 261, 271, 286, 287, 288, 290, 292, 295, 296, 297, 298, 299, 300, 301, 302, 303, 304, 305, 307, 308, 309, 313, 314, 315, 316, 317, 318, 319, 321, 322, 323, 324, 325, 327, 328, 329, 330, 331, 332, 333, 334, 335, 336, 337, 338, 339, 342, 343, 344 and 345 have utility as inhibitors of cruzain and cruzipains with Ki less than 5000 nM.

EXAMPLE 360 was prepared as detailed for EXAMPLE 1 substituting the appropriate carboxylic acids as required and is an inhibitor of Leismania mexicana CPB protease with Ki less than 5000 nM;

### EXAMPLE 360

### (3aR,6aS)-4-tert-Butyl-N-[(1S)-1-(4-methyl-benzyl)-2-oxo-2-(6-oxo-4-phenylacetyl-hexahydro-pyrrolo[3,2-b]pyrrole-1-yl)-ethyl]-benzamide

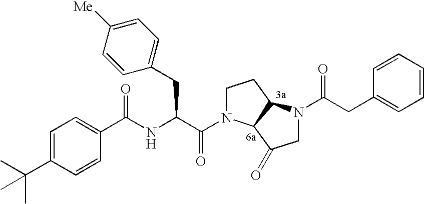

HPLC Rt=19.5-20.6 mins (>80%), HPLC-MS 566.3 [M+H]⁺.

In addition, EXAMPLES 1, 2, 3, 5, 6, 13, 14, 16, 17, 20, 21, 24, 25, 26, 27, 28, 29, 31, 32, 34, 35, 37, 39, 42, 43, 44, 46, 47, 48, 50, 56, 57, 59, 60, 62, 63, 64, 65, 66, 68, 69, 70, 71, 72, 73, 74, 75, 76, 77, 82, 85, 86, 87, 88, 89, 90, 91, 92, 93, 95, 96, 97, 98, 99, 100, 101, 102, 103, 104, 105, 111, 112, 113, 114, 115, 116, 117, 118, 119, 120, 128, 130, 132, 134, 135, 136, 137, 138, 139, 141, 142, 145, 147, 148, 150, 151, 154, 158, 159, 160, 161, 164, 165, 166, 167, 170, 171, 172, 173, 174, 175, 176, 177, 178, 179, 180, 181, 182, 185, 190, 191, 192, 193, 194, 201, 204, 207, 208, 209, 212, 213, 214, 215, 216, 220, 230, 233, 234, 235, 245, 246, 247, 248, 249a, 249b, 249c, 271, 278, 286, 295, 314, 315, 316, 318, 319, 329, 330, 331, 332 and 345 have utility as inhibitors of Leismania mexicana CPB protease with Ki less than 5000 nM.

In addition, EXAMPLES 2, 7, 14, 15, 27, 28, 31, 34, 35, 39, 40, 41, 43, 46, 48, 54, 57, 58, 59, 60, 61, 73, 74, 77, 87, 90, 91, 99, 102, 103, 104, 113, 135, 141, 151, 158, 166, 173, 194, 247, 249a, 249b, 249c, 251, 252, 253, 254, 255, 258, 259, 261, 262, 264, 266, 267, 269, 271, 273, 275, 279, 281, 286, 290, 292, 296, 298, 299, 300, 301, 304, 305, 307, 308, 309, 310, 314, 315, 316, 323, 325, 327, 329 and 330 have utility as inhibitors of cathepsin B with Ki less than 5000 nM.

Solution Phase Syntheses

Alternative strategies to the solid phase techniques described for EXAMPLES 1→360 above are broadly outlined in Schemes 15, 17 and 18. Here, building blocks may be prepared for solution phase syntheses for example 3-Oxo-hexahydro-pyrrolo[3,2-b]pyrrole-1,4-dicarboxylic acid 1-benzyl ester 4-tert-butyl ester (analogue of compound 53, Scheme 15), prepared in 7 steps as follows:

### (1) Preparation of (2S,3S) 3-Hydroxypyrrolidine-1,2-dicarboxylic acid 1-tert-butyl ester

Boc anhydride (2.95 g, 13.5 mmol) was added to a stirred solution of the (2S,3S)-3-hydroxypyrrolidine-2-carboxylic acid (1.61 g, 12.3 mmol) and sodium carbonate (1.3 g, 12.3 mmol) in a mixture of dioxane (25 ml) and water (12.3 ml). The mixture was stirred for 1.5 h at ambient temperature then evaporated under reduced pressure to afford a residue (−10 ml). The residue was diluted with water (30 ml) then extracted with ethyl acetate (40 ml). The aqueous phase was acidified (pH˜2.5) with dilute aqueous hydrochloric acid (0.1 M) then extracted with chloroform (4×50 ml). The combined organic layers were dried (Na₂SO₄) and evaporated under reduced pressure to afford (2S,3S)-3-hydroxypyrrolidine-1,2-dicarboxylic acid 1-tert-butyl ester as a white crystalline solid (2.39 g, ˜85%), HPLC-MS (single main peak, 254.1 [M+Na]⁺ and 485.2 [M+H]⁺).

### (2) Preparation of (2S,3S) 3-Hydroxypyrrolidine-1,2-dicarboxylic acid 2-allyl ester 1-tert-butyl ester

A solution of allyl bromide (26 ml, 301 mmol) and tricaprylmethylammonium chloride (38.4 ml, 86.1 mmol) in dichloromethane (307 ml) was added to a stirred solution of (2S,3S) 3-hydroxypyrrolidine-1,2-dicarboxylic acid 1-tert-butyl ester (19.89 g, 86.1 mmol) and sodium hydrogen carbonate (7.23 g, 86.1 mmol) in water (307 ml). The biphasic mixture was vigorously stirred overnight then diluted with water (100 ml) and the product extracted into dichloromethane (3×200 ml). The combined organic layers were dried (Na₂SO₄) and evaporated under reduced pressure to afford a residue. Flash chromatography of the residue over silica gel (400 g) using ethyl acetate:heptane (1:4) as the eluent afforded (2S,3S) 3-hydroxypyrrolidine-1,2-dicarboxylic acid 2-allyl ester 1-tert-butyl ester (9.4 g, 40%), TLC (single spot, R_{f}=0.28, 50% ethyl acetate in heptane), HPLC-MS (single main peak, 294.1 [M+Na]⁺, 565.3 [2M+Na]⁺); δ_{H} (400 MHz, CDCl₃) 1.41 and 1.46 (combined integration 9H, 2×s, C(CH₃)₃ of geometric isomers), 1.87-1.97 (1H, m, 4-H), 2.06-2.18 (1H, m, 4-H), 2.28-2.36 (1H, m, OH), 3.55-3.71 (2H, m, 5-H₂), 4.20 and 4.32 (combined integration of 1H, 2×s, 2-H geometric isomers), 4.46 (1H, br. s, 3-H), 4.57-4.73 (1H, m, OCH₂), 5.25-5.37 (2H, m, OCH₂CHCH₂) and 5.86-5.98 (1H, m, OCH₂CHCH₂); δ_{C} (100 MHz, CDCl₃) 28.6 and 28.7 (C(CH₃)₃), 32.5 and 32.95 (C-4), 44.5 and 44.9 (C-5), 66.2 (OCH₂), 68.3 (C-2), 74.6 and 75.7 (C-3), 80.6 (OC(CH₃)₃), 118.95 and 119.4 (OCH₂CHCH₂), 131.9 (OCH₂CHCH₂), 154.25 and 154.9 (NCO₂), 170.8 and 171.1 (CO₂Allyl).

### (3) Preparation of (2S,3R) 3-Azido-pyrrolidine-1,2-dicarboxylic acid 2-allyl ester 1-tert-buytl ester

Diethyl azodicarboxylate (0.62 ml, 4.0 mmol) was added dropwise over 25 minutes to a stirred solution of (2S,3S) 3-hydroxypyrrolidine-1,2-dicarboxylic acid 2-allyl ester 1-tert-butyl ester (895 mg, 3.3 mmol) and triphenylphosphine (1.08 g, 4.1 mmol) in tetrahydrofuran (30 ml) at 0° C. under an atmosphere of argon. The solution was stirred for 5 minutes then diphenylphosphoryl azide (0.89 ml, 4.1 mmol) was added dropwise over 10 minutes. The mixture was stirred for 10 minutes at 0° C. then for 24 hours at ambient temperature before removing solvents in vacuo to obtain a residue which was purified by flash chromatography over silica gel eluting with a gradient of heptane:ethyl acetate 3:1→1:1. Appropriate fractions were combined and the solvents removed in vacuo to obtain (2S,3R) 3-azidopyrrolidine-1,2-dicarboxylic acid 2-allyl ester 1-tert-butyl ester as a colourless oil (850 mg, 88%) which was contaminated with 5% of 4,5-dihydropyrrole-1,2-dicarboxylic acid 2-allyl ester 1-tert-butyl ester. TLC (main spot, R_{f}=0.70, heptane:ethyl acetate 1:1), HPLC-MS 197.1 [M−Boc+H]⁺, 241.1 [M−Bu+2H]⁺, 319.2 [M+Na]⁺, 615.3 [2M+Na]⁺; δ_{H} (CDCl₃ at 298 K); (Doubling up of peaks in spectrum due to restricted rotation around Fmoc amide bond) 1.45 and 1.49 (1.8 and 1.2H respectively, Me₃C, each s), 2.10-2.24 (2H, H-4, m), 3.44-3.52 and 3.60-3.73 (each 1H, H-5, m), 4.31-4.40 (1H, H-3, m), 4.45 and 4.54 (0.6 and 0.4H respectively, H-2, each d, J=7.6 Hz), 4.66-4.78 (2H, CH2CH═CH₂, m), 5.28-5.33 (1H, CH₂CH═CH₂, m), 5.42 (1H, CH₂CH═CH₂, dd, J=17.2 and 3.7 Hz), 5.91-6.04 (1H, CH₂CH═CH₂); δ_{C} (CDCl₃ at 298 K); 28.40 (u, Me₃C), 29.40/30.18 (d, C-4), 44.22/44.62 (d, C-5), 61.21/61.86/61.98/62.23 (u, C-2 and C-3), 65.81/66.10 (d, CH₂CH═CH₂), 80.76/81.24 (q, Me₃C), 118.79/119.21 (d, CH₂CH═CH₂), 131.82/131.87 (u, CH₂CH═CH₂), 153.53/154.11 (q, OCON), 169.19/169.38 (q, CO₂CH₂).

### (4) Preparation of (2S,3R) 3-Benzyloxycarbonylaminopyrrolidine-1,2-dicarboxylic acid 1-tert-butyl ester 2-propyl ester

Palladium (10 wt. % on carbon powder, 300 mg) was added portion wise to a solution of (2S,3R) 3-azidopyrrolidine-1,2-dicarboxylic acid 2-allyl ester 1-tert-butyl ester (790 mg, 2.66 mmol) in ethanol (20 ml) at 0° C. The mixture was stirred for 2 h under an atmosphere of hydrogen at ambient temperature, then filtered over celite and concentrated in vacuo to afford a residue (540 mg). The residue was suspended in dioxane (6 ml) then a solution of sodium carbonate (529 mg, 5 mmol) in water (12 ml) added. The mixture was cooled to 0° C. then a solution of benzyloxy chloroformate (0.314 ml) in dioxane (6 ml) added portion wise over 40 min at 0° C. The mixture was stirred for 30 min at 0° C. then at ambient temperature for 40 min. Water (150 ml) was added and the products extracted with chloroform (3×100 ml), dried (Na₂SO₄) and evaporated under reduced pressure to afford a residue. Flash chromatography of the residue over silica (100 g) using ethyl acetate:heptane (1:4) as the eluent afforded (2S,3R) 3-benzyl oxycarbonylaminopyrrolidine-1,2-dicarboxylic acid 1-tert-butyl ester 2-propyl ester (240 mg, 22%), TLC (single spot, R_{f}=0.47, 50% ethyl acetate in heptane); analytical HPLC Rₜ=18.06 min, HPLC-MS (single main peak, 429.2 [M+Na]⁺, 835.4 [2M+Na]⁺); δ_{H} (400 MHz, CDCl₃) 0.90 (3H, t, J=7.3 Hz, CH₃), 1.41 and 1.45 (combined integration 9H, 2×s, C(CH₃)₃ of geometric isomers), 1.46-1.66 (OCH₂CH₂CH₃), 1.89-2.00 (1H, m, 4-H₂), 2.12-2.25 (1H, m, 4-H₂), 3.32-3.46 (1H, m, 5-H), 3.55-3.72 (1H, m, 5-H), 3.99-4.07 (1H, m, 3-H), 4.46-4.55 (OCH₂CH₂CH₃), 4.88-5.17 (combined integration of 3H, m, 2-H and OCH₂Ph), and 7.29-7.42 (6H, m, C₆H₅ and NB); δ_{C} (100 MHz, CDCl₃) 10.5 (CH₃), 22.10 (OCH₂CH₂), 28.41 and 28.50 (C(CH₃)₃), 29.4 and 30.5 (C-4), 43.9 and 44.3 (C-5), 52.8 (C-3), 60.5 and 61.3 (C-2), 66.9, 67.0 and 67.2 (OCH₂Ph and OCH₂), 80.5 (OC(CH₃)₃), 128.3, 128.4 and 128.7 (o-, m- and p-C₆H₅), 136.2 (NHCOOCH₂C), 153.8 and 155.7 (NCO₂ and NHCO₂) and 171.1 (CO₂Propyl).

### (5) Preparation of (2S,3R) 3-Benzyloxycarbonylaminopyrrolidine-1,2-dicarboxylic acid 1-tert-butyl ester

A solution of sodium hydroxide (185 mg, 4.6 mmol) in water (1.6 ml) was added to a solution of (2S,3R) 3-benzyloxycarbonylaminopyrrolidine-1,2-dicarboxylic acid 1-tert-butyl ester 2-propyl ester (229 mg, 0.56 mmol) in ethanol (6 ml). The mixture was stirred for 8 h at ambient temperature. Water (50 ml) was added and the ethanol removed under reduced pressure. The aqueous residue was acidified to pH=2 by the addition of dilute aqueous hydrochloric acid (0.1 M). The mixture was then extracted with ethyl acetate (3×50 ml), dried Na₂SO₄) and evaporated under reduced pressure to afford (2S,3R) 3-benzyloxycarbonylaminopyrrolidine-1,2-dicarboxylic acid 1-tert-butyl ester (200 mg, 98%), analytical HPLC Rₜ=14.03 min, HPLC-MS (single main peak, 387.2 [M+Na]⁺, 751.4 [2M+Na]⁺); δ_{H} (400 MHz, CDCl₃) 1.35 and 1.37 (combined integration 9H, 2×s, C(CH₃)₃ of geometric isomers), 1.86-2.15 (2H, m, 4-H₂), 3.14-3.71 (2H, m, 5-H₂), 4.20-4.49 (2H, m, 2-H and 3-H), 5.02-5.22 (2H, m, OCH₂Ph), and 7.18-7.37 (6H, m, C₆H₅ and NH); δ_{C} (100 MHz, CDCl₃) 28.3, 28.4, 28.5 and 29.2 (C(CH₃)₃ and C-4), 43.8 and 44.3 (C-5), 52.3 and 53.1 (C-3), 61.0 and 61.4 (C-2), 67.2 and 68.4 (OCH₂Ph), 80.6 and 80.8 (OC(CH₃)₃), 128.2, 128.5 and 128.6 and 128.7 (o-, m- and p-C₆H₅), 135.5 (NHCOOCH₂C), 153.8 and 154.3 (NCO₂), 158.6 (NHCO₂), 175.6 and 175.9 (CO₂H).

### (6) Preparation of (2S,3R) 3-Benzyloxycarbonylamino-2-(2-diazoacetyl) pyrrolidine-1-carboxylic acid tert-butyl ester

(2S,3R) 3-benzyloxycarbonylaminopyrrolidine-1,2-dicarboxylic acid 1-tert-butyl ester (161 mg, 0.443 mmol) was dissolved with stirring in anhydrous dichloromethane (18 ml). The reaction was flushed with nitrogen and cooled to −15° C. iso-Butylchloroformate (0.063 ml, 0.487 mmol) in anhydrous dichloromethane (1.5 ml) and N-methylmorpholine (0.097 ml, 0.886 mmol) in anhydrous dichloromethane (1.5 ml) were added simultaneously in 0.5 ml aliquots over 15 min. The mixture was stirred for 45 min at −15° C. then ethereal diazomethane (generated from addition of diazald (4.7 g, ˜15 mmol) in diethyl ether (75 ml) onto sodium hydroxide (5.25 g) in water (7.5 ml)/ethanol (15 ml) at 60° C.] was added to the activated amino acid solution. The mixture was allowed to warm to ambient temperature and stirred for 24 h. A few drops of acetic acid were added to the mixture, followed by diethyl ether (200 ml). The ethereal layer was washed with saturated aqueous sodium hydrogen carbonate (80 ml), dried (Na₂SO₄) and the solvents removed under reduced pressure to give a yellow residue (300 mg). Flash chromatography of the residue over silica (35 g) using ethyl acetate:heptane (1:3) afforded (2S,3R) 3-benzyloxycarbonylamino-2-(2-diazoacetyl)pyrrolidine-1-carboxylic acid tert-butyl ester (150 mg, 87%), TLC (single spot, R_{f}=0.29, 50% ethyl acetate in heptane); analytical HPLC Rt=15.15 min, HPLC-MS (single main peak, 411.2 [M+Na]⁺, 799.4 [2M+Na]⁺).

### (7) Preparation of (2S,3R) 3-Oxohexahydropyrrolo[3,2-b]pyrrole-1,4-dicarboxylic acid 1-benzyl ester 4-tert-butyl ester

A solution of (2S,3R) 3-benzyloxycarbonylamino-2-(2-diazoacetyl) pyrrolidine-1-carboxylic acid tert-butyl ester (90 mg, 0.23 mmol) was added over 1 h to a refluxing solution of rhodium(II) acetate dimer (2 mg, 0.0046 mmol) in dichloromethane (3 ml). The mixture was heated under reflux for 2 h and the solvent then removed in vacuo. The residue was diluted with tetrahydrofuran (40 ml) then filtered through a pad of celite. The filtrate was then concentrated in vacuo to afford a residue (106 mg). Flash chromatography of the residue over silica (35 g) using ethyl acetate:heptane (1:3) afforded (2S,3R) 3-oxo-hexahydropyrrolo[3,2-b]pyrrole-1,4-dicarboxylic acid 1-benzyl ester 4-tert-butyl ester (14 mg, 17%), TLC (two possible rotameric spots, R_{f}=0.30 and 0.23, 50% ethyl acetate in heptane), analytical HPLC Rₜ=15.479 min (−60%), 16.393 (−10%), 17.197 (˜10%), 18.085 (˜10%) and 21.665 (−10%); HPLC-MS (4 main peaks, 361.0=[M+H]⁺, 379=[M+H₃O]⁺, 401.0 [M+H₂O+Na]⁺, [2M+H]⁺=721.0, [2M+Na]⁺=743.0, [2[M+H₂O]^{+Na]}⁺=779.0); δ_{H} (500 MHz, CDCl₃) 1.40-1.50 (9H, m, C(CH₃)₃), 1.78-1.86 (1H, m, NCH₂CH₂), 2.30-2.47 (1H, m, NCH₂CH₂), 3.90-4.06 (2H, m, NCH₂), 5.05-5.20 (4H, m, OCH₂, BocNCHCO and CbzNCH), 5.44 (1H, d, J=2.2 Hz, CbzNCH₂), 5.56 (1H, br. s, CbzNCH₂) and 7.30-7.37 (5H, m, C₆H₅); δ_{C} (126 MHz, CDCl₃) 27.5 (C(CH₃)₃), 29.6 and 32.2 (NCH₂CH₂), 57.7 (CbzNCH), 59.1 (NCH₂), 67.0 (OCH₂), 77.2 (BocNCH), 83.5 (OC(CH₃)₃), 110.7 (CbzNCH₂), 128.0, 128.2 and 128.5 (o-, m- and p-C₆H₅), 136.1 (OCH₂C of Cbz), 147.6, 151.4 155.7 and 167.1 (2×NCO₂ and CbzNCH₂CO).

### (8) Preparation of (2S,3R) 3-Oxohexahydropyrrolo[3,2-b]pyrrole-1-carboxylic acid benzyl ester hydrochloride salt (analogue of compound 54)

(2S,3R) 3-oxo-hexahydropyrrolo[3,2-b]pyrrole-1,4-dicarboxylic acid 1-benzyl ester 4-tert-butyl ester (9 mg, 0.025 mmol) was dissolved in 4M HCl in dioxane (1.25 ml) then stirred for 1 h at room temperature. The mixture was concentrated in vacuo then toluene (10 ml) added. The mixture was concentrated once again in vacuo and the procedure repeated to afford a residue (10 mg, ˜100%), HPLC-MS (single main peak, 261.1 [M+H]⁺ and 279.1 [M+H₃O]⁺).

Alternatively, a useful building block for solution phase synthesis is (3S,3aS,6aR)-3-hydroxyhexahydropyrrolo[3,2-b]pyrrole-1,4-dicarboxylic acid 4-benzyl ester 1-tert-butyl ester (80) described earlier in Scheme 20. The utility of building block (80) is detailed in alternative syntheses of EXAMPLES 1 and 14, through Scheme 26, which is an example of the general synthetic strategy detailed in Scheme 18.

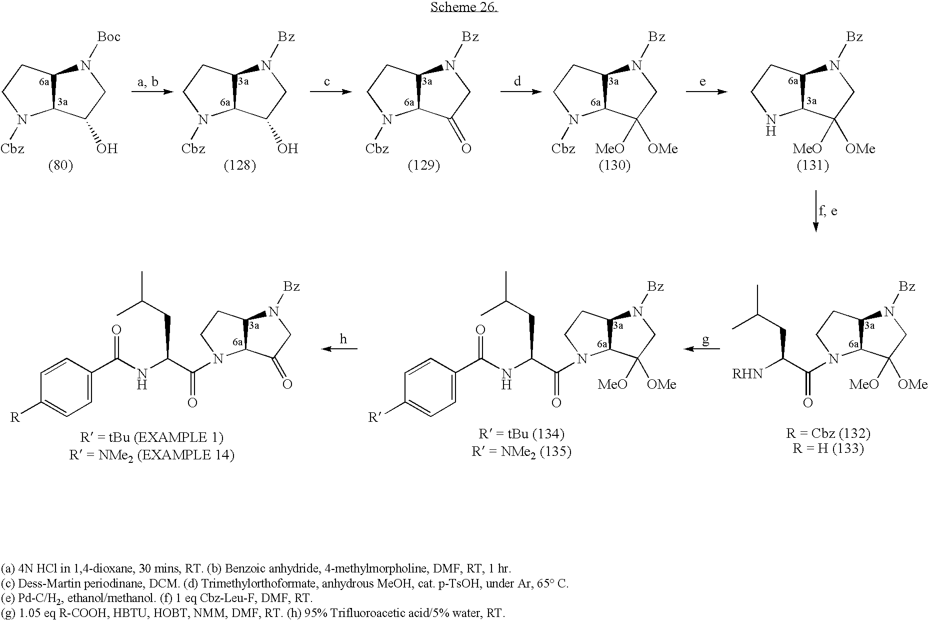

### Preparation of (3aR,6S,6aS)-benzoyl-6-hydroxyhexahydropyrrolo[3,2-b]pyrrole-1-carboxylic acid benzyl ester (128)

A solution of HCl in 1,4-dioxane (4.0M, 11 ml, 44 mmol) was added to (3S,3aS,6aR)-3-hydroxyhexahydropyrrolo[3,2-b]pyrrole-1,4-dicarboxylic acid 4-benzyl ester 1-tert-butyl ester (80) (450 mg, 1.24 mmol). The solution was stirred for 65 minutes whereupon a white suspension formed. The solvents were removed in vacuo and the residue azeotroped with diethyl ether (3×15 ml) and then dimethylformamide (10 ml) and benzoic anhydride (295 mg, 1.31 mmol) added. The solution was placed under an atmosphere of argon then 4-methylmorpholine (0.29 ml, 2.6 mmol) was added to the solution dropwise whilst stirring over 0.5 minutes. The mixture was stirred for 1.75 hours then the solvents were removed in vacuo. The residue was dissolved in ethyl acetate (100 ml) then washed with saturated aqueous sodium hydrogen carbonate solution (100 ml), pH 3 hydrochloric acid (100 ml) then brine (100 ml). The organic layer was dried (Na₂SO₄) and evaporated in vacuo to afford (3aR,6S,6aS)-4-benzoyl-6-hydroxyhexahydropyrrolo[3,2-b]pyrrole-1-carboxylic acid benzyl ester (128) as a pale yellow gum (465 mg), which was used without further purification. Analytical HPLC Rₜ=14.967 min; HPLC-MS 367.1 [M+H]⁺, 733.1 [2M+H]⁺; Elemental analysis C₂₁H₂₂N₂O₄.0.4EtOAc req. (fnd.) % C 67.62 (67.73), % H 6.33 (6.17), % N 6.98 (7.08); HRMS C₂₁H₂₂N₂O₄Na req. 389.1477, fnd. 389.1476 (−0.40 ppm); d_{H} (500 MHz, CDCl₃) approximately 3:1 mixture of rotamers, 2.10-2.21 (1H, m, BzNCHCH₂), 2.24-2.36 (1H, m, BzNCHCH₂), 3.20-3.35 (1H, m, CbzNCH₂), 3.35-3.66 (2H, m, BzNCH₂), 3.74-3.80 (1H, m, CbzNCH₂), 4.16-4.20 (1H, m, CbzNCR), 4.38-4.42 (1H, br. s; CHOH), 4.94-5.04 (1H, m, BzNCB), 5.08-5.22 (2H, m, OCH₂Ph), 7.30-7.52 (10H, aromatic CB); d_{C} (125 MHz, CDCl₃) 31.02, 30.40 (BzNCHCH₂), 45.73, 45.86 (CbzNCH₂), 56.43, 56.74 (BzNCH₂), 60.58, 61.49 (BzNCH), 66.68, 67.33 (OCH₂Ph), 66.92, 67.76 (CbzNCH), 73.01, 73.86 (CHOH), 126.77, 127.48, 127.99, 128.23, 128.28, 128.45, 128.56, 128.87, 130.02, 130.38 and 134.53 (CH aromatics), 136.16, 136.23, 136.33 (aromatic quaternary), 154.26, 154.97 (CbzC═O), 170.06, 171.19 (BzC═O).

### Preparation of (3aR,6aS)-4-benzoyl-6-oxo-hexahydropyrrolo[3,2-b]pyrrole-1-carboxylic acid benzyl ester (129)

(3aR,6S,6aS)-4-Benzoyl-6-hydroxyhexahydropyrrolo[3,2-b]pyrrole-1-carboxylic acid benzyl ester (128) (0.78 g, 2.13 mmol) was dissolved in dichloromethane (20 ml) with stirring under argon. Dess-Martin periodinane (1.804 g, 4.26 mmol) was added and the mixture stirred for 16 hours. The mixture was concentrated in vacuo and the residue purified by flash chromatography over silica, eluting with ethyl acetate:heptane mixtures to give (3aR,6aS)-4-benzoyl-6-oxo-hexahydropyrrolo[3,2-b]pyrrole-1-carboxylic acid benzyl ester (129) (0.61 g, 78%) as an off-white gum. TLC (R_{f}=0.27, EtOAc:heptane 3:1), analytical HPLC single main peak, Rₜ=14.65-16.30 min., HPLC-MS 365.1 [M+H]⁺, 383.1 [M+H+H₂O]⁺; Elemental analysis C₂₁H₂₀N₂O₄.1.2H₂O req. (fnd.) % C 65.38 (65.12), % H 5.85 (5.65), % N 7.26 (6.95>; HRMS C₂₁H₂₀N₂O₄Na req. 387.1321, fnd. 387.1324 (0.76 ppm); Sc (125 MHz, CDCl₃) 30.41, 30.89, 31.23 (BzNCHCH₂), 45.75 (CbzNCH₂), 54.55, 63.04 (BzNCH+CbzNCH), 57.91, 58.45, 58.99, 59.73 (BzNCH₂), 67.60, 68.07 (OCH₂Ph), 127.00, 127.38, 127.48, 127.98, 128.11, 128.48, 128.62, 128.74, 130.48, 130.83 (CH aromatics), 135.07, 136.14 (quaternary aromatics), 154.54, 155.03 (CbzCO₂), 170.58 (BzCO), 204-207 (broad, C═O).

### Preparation of (3aR,6aS)-4-benzoyl-6,6-dimethoxyhexahydropyrrolo[3,2-b]pyrrole-1-carboxylic acid benzyl ester (130)

(3aR,6aS)-4-Benzoyl-6-oxo-hexahydropyrrolo[3,2-b]pyrrole-1-carboxylic acid benzyl ester (129) (0.60 g, 1.65 mmol) was dissolved in methanol (10 ml) with stirring. Trimethylorthoformate (1.8 ml, 16.5 mmol) was added followed by para-toluenesulfonic acid (40 mg) and the mixture heated under argon at 65° C. for 16 hours. The mixture was reduced in vacuo to leave a dark oil (0.8 g). Flash chromatography over silica, eluting with ethyl acetate:heptane mixtures gave (3aR,6aS)-4-benzoyl-6,6-dimethoxyhexahydropyrrolo[3,2-b]pyrrole-1-carboxylic acid benzyl ester (130) (0.52 g, 77%) as a fine white crystalline solid. TLC (R_{f}=0.40, EtOAc:heptane 3:1), analytical HPLC single main peak, Rₜ=18.22 min., HPLC-MS 411.1 [M+H]⁺, 433.1 [M+Na]⁺, 843.1 [2M+Na]⁺; HRMS C₂₃H₂₆N₂O₅Na req. 433.1739, fnd. 433.1727 (−2.94 ppm); δ_{H} (500 MHz, CDCl₃) 1.96-2.07, 2.15-2.22 (2H, m, BzNCHCH₂), 3.04-3.42 (6H, m, 2×OCH₃), 3.25 (1H, m, CbzNCH₂), 3.4 (1H, m, BzNCH₂), 3.58-3.67 (1H, m, BzNCH₂), 3.96-4.07 (1H, m, CbzNCH₂), 4.35-4.58 (1H, m, CbzNC), 4.98-5.26 (3H, BzNCH+OCH₂Ph), 7.28-7.49 (10H aromatics); δ_{C} (125 MHz, CDCl₃) 32.27, 32.59 (BzNCHCH₂), 46.74 (CbzNCH₂), 49.36, 51.10, 51.59 (2×OCH₃), 54.59, 56.08 (BzNCH₂), 60.77, 61.08 (BzNCH), 62.47 (CbzNCH), 67.28 (OCH₂Ph), 106.76, 107.02 (C(OCH₃)₂), 126.84, 127.35, 127.90, 128.06, 128.39, 130.05, 130.38 (CH aromatics), 135.91, 136.48 (quaternary aromatics), 155.44 (CbzCO₂), 169.54 (BzCO).

### Preparation of (3aS,6aR)-(3,3-dimethoxyhexahydropyrrolo[3,2-b]pyrrol-1-yl)phenylmethanone (131)

Methanol (15 ml) was added cautiously dropwise to a stirred mixture of (3aR,6aS)-4-benzoyl-6,6-dimethoxyhexahydropyrrolo[3,2-b]pyrrole-1-carboxylic acid benzyl ester (130) (0.48 g, 1.17 mmol) and 10% palladium on charcoal (100 mg) at 0° C. under an atmosphere of argon over 10 minutes. The argon was then replaced by an atmosphere of hydrogen and stirring continued at ambient temperature for 85 minutes before replacing the hydrogen with argon and adding ethanol (30 ml). The mixture was filtered under reduced pressure through celite and the filter cake washed with methanol (25 ml) then ethanol (70 ml). Solvents were removed from the filtrate in vacuo to obtain (3aS,6aR)-(3,3-dimethoxyhexahydropyrrolo[3,2-b]pyrrole-1-yl) phenylmethanone (131) as a colourless oil (340 mg), which was used without further purification. HPLC-MS 277.1 [M+H]⁺, 553.2 [2M+H]⁺.

### Preparation of (3aR,6aS)-[(1S)-1-(4-benzoyl-6,6-dimethoxyhexahydropyrrol [3,2-b]pyrrole-1-carbonyl)-3-methylbutyl]carbamic acid benzyl ester (132)

(i) Preparation of Cbz-L-Leucine Fluoride

Cbz-L-Leucine (1.115 g, 4.2 mmol) was dissolved in dichloromethane (50 ml) with stirring under argon. (Diethylamino)sulfur trifluoride (DAST, 792 μl, 6.0 mmol) was added and the mixture stirred for 40 minutes. Ice-cooled water (200 ml) was added to the mixture and the organic layer separated, dried (Na₂SO₄) and reduced in vacuo to a mobile tan oil (1.14 g). An analytical sample, pre-treated with 10% pyridine in methanol for 15 minutes gave HPLC-MS 266.1 [M+H]⁺ (acid, <5%), 280.1 [M+H]⁺, 302.1 [M+Na]⁺, 581.1 [2M+Na]⁺ (methyl ester, ˜95%).

(ii) Crude (3aS,6aR)-(3,3-dimethoxyhexahydropyrrolo[3,2-b]pyrrole-1-yl)phenyl methanone (131) (˜1.17 mmol) was dissolved in anhydrous dimethylformamide (5 ml) with stirring. Cbz-L-Leucine fluoride (0.33 g, 1.23 mmol) was added and the mixture stirred under argon for 1 hour. The mixture was reduced in vacuo to a semi-mobile dark oil (˜1.0 g). Flash chromatography over silica, eluting with ethyl acetate:heptane mixtures gave (3aR,6aS)-[(1S)-1-(4-benzoyl-6,6-dimethoxyhexahydro pyrrolo[3,2-b]pyrrole-1-carbonyl)-3-methylbutyl]carbamic acid benzyl ester (132) (0.55 g, 90%) as an off-white crystalline solid. TLC (R_{f}=0.35, EtOAc:heptane 3:1), analytical HPLC single main peak, Rₜ=19.396 min., HPLC-MS 524.1 [M+H]⁺, 546.1 [M+Na]⁺, 1069.2 [2M+Na]⁺; Elemental analysis C₂₉H₃₇N₃O₆ req. (fnd.) % C 66.52 (66.26), % H 7.12 (7.30), % N 8.02 (7.86); HRMS C₂₉H₃₇N₃O₆Na req. 546.2580, fnd. 546.2584 (0.67 ppm); δ_{H} (500 MHz, CDCl₃) 0.92-1.04 (6H, m, 2× Leu δCH₃), 1.45-1.55 (2H, m, Leu βCH₂), 1.73-1.84 (1H, m, Leu γCH), 1.92-1.99, 2.10-2.16, 2.22-2.30 (2H, 4:6:10, m, BzNCHCH₂), 2.94, 3.19, 3.23 and 3.40 (6H total, each s, C(OCH₃)₂), 3.14-3.38 (2H, m, 1×BzNCH₂ and 1×CbzLeuNCH₂), 3.60-3.68 (1H, 4:6, each d, J=12 Hz, 1×BzNCH₂), 4.03-4.10, 4.11-4.18 (1H, 4:6, m, 1×CbzLeuNCH₂), 4.33 (0.4H, d, J=6.3 Hz, 0.4×CbzLeuNCE), 4.5-4.65 (0.6H, m, 0.6× Leu aCH), 4.82 (0.6H, d, J=6.45 Hz, 0.6×CbzLeuNCH), 4.87-4.93 (0.4H, m, 0.4× Leu aCH), 5.0-5.14 (3H, m, BzNCH+OCH₂Ph), 5.42 (0.6H, d, J=8.3 Hz, LeuNH), 5.57 (0.4H, d, J=8.9 Hz, LeuNH), 7.3-7.5 (10H, aromatics); δ_{C} (125 MHz, CDCl₃) 21.99, 22.20, 22.67, 23.06, 23.67 (2× Leu δCH₃), 24.37, 24.57 (Leu γCH), 31.58, 31.86, 33.26 (BzNCHCH₂), 42.78 (Leu βCH₂), 44.12, 45.79, 47.05 (CbzLeuNCH₂), 49.31, 49.99 (1×OCH₃), 51.18, 51.27, 51.30, 51.47 (1×OCH₃+Leu αCH), 55.55, 57.03 (BzNCH₂), 59.69, 61.32 (BzNCH), 60.30, 61.04 (CbzLeuNCH), 66.39, 66.88(OCH₂Ph), 106.27, 107.11 (C(OCH₃)₂), 126.84, 127.32, 127.42, 127.87, 127.94, 128.09, 128.43, 128.48, 130.18, 130.43, 130.50 (CH aromatics), 135.68, 135.81, 136.28, 136.73 (quaternary aromatics), 155.58, 156.21 (CbzCO₂), 169.56, 169.62 (BzCO), 172.35, 173.36 (Leu C═O).

### Preparation of (2S,3aR,6aS)-2-amino-1-(4-benzoyl-6,6-dimethoxyhexahydro pyrrolo[3,2-b]pyrrole-1-yl)-4-methylpentan-1-one (133)

Methanol (15 ml) was added cautiously dropwise to 10% palladium on charcoal (75 mg) at 0° C. under an atmosphere of argon over 10 minutes whilst stirring followed by a solution of (3aR,6aS)-[(1S)-1-(4-benzoyl-6,6-dimethoxy hexahydropyrrolo[3,2-b]pyrrole-1-carbonyl)-3-methylbutyl]carbamic acid benzyl ester (132) (0.52 g, 0.99 mmol) in methanol (15 ml). The argon was then replaced by an atmosphere of hydrogen and stirring continued at ambient temperature for 5.5 hours. A suspension of 10% palladium on charcoal (15 mg) in methanol (1 ml) was added and stirring continued for 2.25 hours. The hydrogen was replaced by argon then ethanol (100 ml) was added before filtering the mixture through celite. The filter cake was washed with ethanol (100 ml) then the filtrate separated into two equal portions before concentrating separately in vacuo to obtain two identical batches of (2S,3aR,6aS)-2-amino-1-(4-benzoyl-6,6-dimethoxy hexahydropyrrolo[3,2-b]pyrrole-1-yl)-4-methyl pentan-1-one (133). TLC (Single spot, R_{f}=0.05, EtOAc:heptane 9:1), HPLC-MS 390.2 [M+H]⁺, 801.2 [2M+Na]⁺ as white solids which were contaminated with approximately 5% of (3aR,6aS)-[(1S)-1-(4-benzoyl-6,6-dimethoxyhexahydropyrrolo[3,2-b]pyrrole-1-carbonyl)-3-methylbutyl]carbamic acid benzyl ester (132) starting material. Each batch was used without further purification (see preparations of (134) and (135) below).

### Preparation of (3aR,6aS)-N-[(1S)-1-(4-benzoyl-6,6-dimethoxy hexa hydropyrrolo[3,2-b]pyrrole-1-carbonyl)-3-methylbutyl]-4-tert-butyl benzamide (134)

4-Methylmorpholine (0.109 ml, 0.994 mmol) was added to a solution of HBTU (189 mg, 0.497 mmol), 1-hydroxybenzotriazole monohydrate (76 mg, 0.497 mmol) and 4-(tert-butyl)benzoic acid (88 mg, 0.497 mmol) in dimethylformamide (12.5 ml). The solution was stood for 5 minutes then added to (2S,3aR,6aS)-2-amino-1-(4-benzoyl-6,6-dimethoxyhexahydropyrrolo[3,2-b]pyrrole-1-yl)-4-methylpentan-1-one (133) (prepared as above, 0.497 mmol). The mixture was stirred at ambient temperature for 1 hour 50 minutes then the solvents were removed in vacuo. The residue was dissolved in dichloromethane (60 ml) then washed with pH 3 hydrochloric acid (40 ml), saturated aqueous sodium hydrogen carbonate solution (40 ml) and brine (40 ml), then dried (Na₂SO₄) and the solvents removed in vacuo. The residue was purified by flash chromatography over silica eluting with ethyl acetate:heptane mixtures 0:100 to 50:50 to give (3aR,6aS)-N-[(1S)-1-(4-benzoyl-6,6-dimethoxyhexahydro pyrrolo [3,2-b]pyrrole-1-carbonyl)-3-methylbutyl]-4-tert-butyl benzamide (134) as a white solid (230 mg) which contained approximately 5% of (3aR,6aS)-[(1S)-1-(4-benzoyl-6,6-dimethoxyhexahydropyrrolo[3,2-b]pyrrole-1-carbonyl)-3-methylbutyl] carbamic acid benzyl ester (132) The latter compound was removed prior to the hydrolysis step (i.e. preparation of EXAMPLE 1 below) by dissolving (220 mg) in methanol (11 ml) then adding to a stirred suspension of 10% palladium on charcoal (70 mg) in ethanol (11 ml) under an atmosphere of argon at 0° C. The argon was then replaced by hydrogen and the mixture stirred at ambient temperature for 80 minutes, then water (11 ml) was added and the mixture filtered through celite. The filter cake was washed with ethanol (200 ml) then the filtrate concentrated in vacuo to give an oily solid (3aR,6aS)-N-[(1S)-1-(4-benzoyl-6,6-dimethoxyhexahydropyrrolo[3,2-b]pyrrole-1-carbonyl)-3-methylbutyl]-4-tert-butylbenzamide (134) (203 mg) which was used without her purification. TLC (Single spot, R_{f}=0.65, EtOAc in heptane 9:1), analytical HPLC Rₜ=21.412 min; HPLC-MS 550.2 [M+H]⁺; Elemental analysis C₃₂H₄₃N₃O₅ req. (fnd.) % C 69.92 (69.52), % H 7.88 (8.12), % N 7.64 (7.40); HRMS C₃₂H₄₄N₃O₅ req. 550.3281, fnd. 550.3284 (0.55 ppm).

### Preparation of (3aR,6aS)-N-[(1S)-1-(4-benzoyl-6,6-dimethoxyhexahydro pyrrolo[3,2-b]pyrrole-1-carbonyl)-3-methylbutyl]-4 dimethylaminobenzamide (135)

4-Methylmorpholine (0.109 ml, 0.994 mmol) was added to a solution of HBTU (189 mg, 0.497 mmol), 1-hydroxybenzotriazole monohydrate (76 mg, 0.497 mmol) and 4-(dimethylamino)benzoic acid (82 mg, 0.497 mmol) in dimethylformamide (12.5 ml). The solution was stood for 5 minutes then added to (2S,3aR,6aS)-2-amino-1-(4-benzoyl-6,6-dimethoxyhexahydropyrrolo[3,2-b]pyrrol-1-yl)-4-methylpentan-1-one (133) (prepared as above, 0.497 mmol). The mixture was stirred at ambient temperature for 7 hours then the solvents removed in vacuo (water bath temperature<26° C.) to obtain a volume of approximately 3 ml. Stirring was continued for 1.25 hours then the remaining solvent was removed in vacuo. The residue was dissolved in dichloromethane (60 ml) then washed with pH 3 hydrochloric acid (40 ml), saturated aqueous sodium hydrogen carbonate solution (40 ml) and brine (40 ml), then dried (Na₂SO₄) and the solvents removed in vacuo. The residue was purified by flash chromatography over silica eluting with ethyl acetate:heptane mixtures 0:100 to 65:35 to give (3aR,6aS)-N-[(1S)-1-(4-benzoyl-6,6-dimethoxyhexahydropyrrolo[3,2-b]pyrrole-1-carbonyl)-3-methylbutyl]-4-dimethylamino benzamide (135) as a white solid (180 mg, 68%). TLC (Single spot, R_{f}=0.30, EtOAc:heptane 9:1), analytical HPLC Rₜ=15.789 min; HPLC-MS 537.2 [M+H]⁺; Elemental analysis C₃₀H₃₀N₄O₅.0.4EtOAc req. (fnd.) % C 66.41 (66.60), % H 7.62 (7.92), % N 9.80 (9.51); HRMS C₃₀H₄₀N₄O₅Na req. 559.2896, fnd. 559.2902 (0.95 ppm).

### Preparation of (3aR,6aS)-N-[(1S)-1-(4-benzoyl-oxo-hexahydropyrrolo[3,2-b]pyrrole-carbonyl)-3-methylbutyl]-4-tert-butylbenzamide (EXAMPLE 1)

(3aR,6aS)-N-[(1S)-1-(4-Benzoyl-6,6-dimethoxyhexahydropyrrolo[3,2-b]pyrrole-1-carbonyl)-3-methyl butyl]-4-tert-butylbenzamide (134) (0.19 g, 0.345 mmol) was dissolved in ice-cooled trifluoroacetic acid/water (95:5 v/v, 10 ml) with stirring. The ice-bath was removed and the mixture stirred at ambient temperature for 3.5 hours. The mixture was then reduced in vacuo and evaporated from diethyl ether (2×10 ml) to give a semi-mobile tan gum (0.3 g). Flash chromatography over silica, eluting with ethyl acetate:heptane mixtures gave (3aR,6aS)-N-[(1S)-1-(4-benzoyl-6-oxo-hexahydropyrolo[3,2-b]pyrrole-1-carbonyl)-3-methylbutyl]-4-tert-butyl benzamide (EXAMPLE 1) (0.042 g, 24%) as a white solid. TLC (R_{f}=0.45, EtOAc:heptane 9:1), analytical HPLC single broad main peak, Rₜ=19.43-21.37 min., HPLC-MS 504.1 [M+H]⁺; Elemental analysis C₃₀H₃₇N₃O₄.0.5TFA req. (fnd.) % C 66.45 (66.04), % H 6.75 (7.19), % N 7.50 (7.24); HRMS C₃₀H₃₇N₃O₄Na req. 526.2682, fnd. 526.2677 (−0.96 ppm); d_{H} (500 MHz, CDCl₃) Tentative assignment of peaks due to presence of rotamers 0.95 (3H, d, J=6.5 Hz, Leu δCH₃), 1.01 (3H, d, J=6.2 Hz, Leu δCH₃), 1.31 (9H, s, C(CH₃)₃), 1.58-1.81 (3H, m, Leu βCH₂ and Leu γCH), 1.85-2.73 (2H, m, BzNCHCH₂), 3.55-3.69 (1H, m, BzNCHCH₂CH₂), 3.85-5.20 (6H, m, BzNCHCH₂CH₂, BzNCH₂C(═O)CH and Leu αCH), 6.70-6.89 (1H, m, NH), 7.40-7.52 (7H, m, COC₆H₅ and CHCHCC(CH₃)₃), 7.65-7.76 (2H, m, CHCHCC(CH₃)₃); δ_{C} (500 MHz, CDCl₃) 22.06, 23.28 (2× Leu δCH₃), 24.82 (Leu γCH), 31.11, 31.14 (C(CH₃)₃), 31.67, 31.86 (BzNCHCH₂), 34.89, 34.92 (C(CH₃)₃), 42.43 (Leu βCH₂), 46.10 (BzNCHCH2CH₂), 48.93 (Leu αCH), 60.2 (BzNCH₂), 61.0 (BzNCH or BzNCH₂C(═O)CH), 68.2 (BzNCH or BzNCH₂C(═O)CH), 125.41, 125.49, 125.54, 126.91, 126.97, 127.11, 127.46, 128.33, 128.79, 130.84 (CH aromatics), 130.70, 131.16, 135.0 (quaternary aromatics), 155.35 (CC(CH₃)₃), 167.07, 170.67, 172.61 (3×NC═O).

### Preparation of (3aR,6aS)-N-[(1S)-1-(4-benzoyl-6-oxo-hexahydropyrrolo[3,2-b]pyrrol-1-carbonyl)-3-methylbutyl]-4-dimethylamiobenzamide (EXAMPLE 14)

Water (1.75 ml) was added dropwise to a stirred solution of (3aR,6aS)-N-[(1S)-1-(4-benzoyl-6,6-dimethoxyhexahydropyrrolo[3,2-b]pyrrole-1-carbonyl)-3-methylbutyl]-4-dimethylaminobenzamide (135) (175 mg, 0.327 mmol) in trifluoroacetic acid (17.5 ml) at 0° C. over 3 minutes. The solution was then stirred at ambient temperature for 17 hours then the solvents removed in vacuo (water bath<25° C.). The residue was azeotroped with diethyl ether (25 ml) then the residue purified by flash chromatography over silica eluting with ethyl acetate: heptane mixtures 0:100 to 70:30 to give (3aR,6aS)-N-[(1S)-1-(4-benzoyl-6-oxo-hexahydropyrrolo[3,2-b]pyrrole-1-carbonyl)-3-methylbutyl]-4-dimethylaminobenzamide (EXAMPLE 14) as a white solid (113.6 mg, 71%). TLC (Single spot, R_{f}=0.25, EtOAc:heptane 9:1), analytical HPLC Rₜ=14.241 min; HPLC-MS 491.2 [M+H]⁺; C₂₈H₃₄N₄O₄.0.5TFA req. (fnd.) % C 63.64 (63.07), % H 6.35 (6.76), % N 10.23 (9.91); HRMS C₂₈H₃₄N₄O₄Na req. 513.2478, fnd. 513.2492 (2.72 ppm); d_{H} (500 MHz, CDCl₃) Tentative assignment of peaks due to presence of rotamers 0.94 (3H, d, J=6.5 Hz, Leu δCH₃), 0.99 (3H, d, J=6.2 Hz, Leu δCH₃), 1.46-1.83 (3H, m, Leu βCH₂ and Leu γCH), 1.90-2.70 (2H, m, BzNCHCH₂), 2.99 (6H, s, N(CH₃)₂), 3.45-3.69 (1H, m, BzNCHCH₂CH₂), 3.90-5.25 (6H, m, BzNCHCH₂CH₂, BzNCH₂C(═O)CH and Leu aCH), 6.58-6.75 (1H, m, NH), 6.60-6.68 (2H, m, CHCN(CH₃)₂), 7.35-7.55 (5H, m, COC₆H₅), 7.60-7.73 (2H, m, CHCHCN(CH₃)₂); δ_{C} (500 MHz, CDCl₃) 22.13, 23.25 (2× Leu δCH₃), 24.78 (Leu γCH), 31.65, 31.85 (BzNCHCH₂), 40.04, 40.09, 40.13 (N(CH₃)₂), 42.35 (Leu γCH₂), 46.12 (BzNCHCH₂CH₂), 48.79 (Leu αCH), 59.99 (BzNCH₂), 60.80 (BZNCH or BzNCH₂C(═O)CH), 67.7 (BzNCH or BzNCH₂C(═O)CH), 110.89, 111.00, 111.04, 111.35 (CHCN(CH₃)₂), 120.17 (CCHCHCN(CH₃)₂), 126.92, 127.12, 127.48, 128.31, 128.63, 128.67, 128.77, 129.00, 130.81, 130.96 (CH aromatics), 135.0 (quaternary aromatics), 152.61 (CN(CH₃)₂), 167.21, 170.69, 173.01 (3×NC═O).

Alternatively, a useful building block for solution phase synthesis is (3aR,6S,6aS)-4-benzoyl-6-hydroxyhexahydropyrrolo[3,2-b]pyrrole-1-carboxylic acid benzyl ester (128) described earlier in Scheme 26. The utility of building block (128) is detailed in an alternative synthesis of EXAMPLE 1, through Scheme 27, which is an example of the general synthetic strategy detailed in Scheme 17.

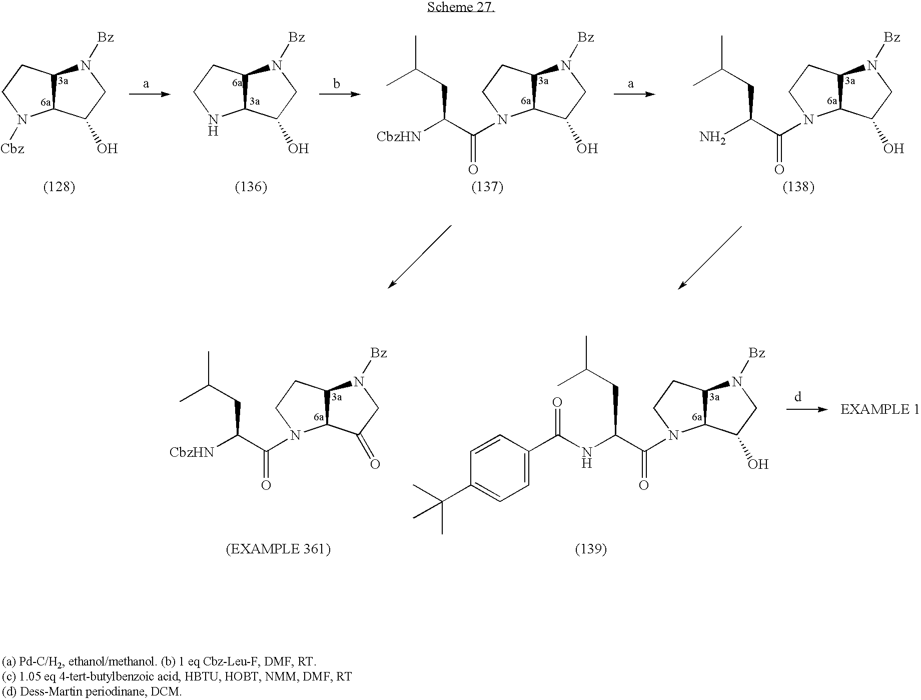

### Preparation of (3S,3aS,6aR)-(3-hydroxyhexahydropyrrolo[3,2-b]pyrrol-1-yl)phenylmethanone (136)

Ethanol (5 ml) was added cautiously dropwise to 10% palladium on charcoal (50 mg) at 0° C. under an atmosphere of argon over 10 minutes whilst stirring followed by a solution of (3aR,6S,6aS)-4-benzoyl-6-hydroxy hexahydropyrrolo[3,2-b]pyrrole-1-carboxylic acid benzyl ester (128) (465 mg, 1.27 mmol prepared as above) in ethanol (10 ml). The argon was then replaced by an atmosphere of hydrogen and stirring continued at ambient temperature for 4.5 hours. The hydrogen was then replaced by argon and 10% palladium on charcoal (20 mg) was added at 0° C. The argon was then replaced with hydrogen and stirring was continued for 4 hours. The hydrogen was replaced by argon then the mixture was filtered through celite. The filter cake was washed with ethanol (75 ml) then the filtrate concentrated in vacuo to obtain (3S,3aS,6aR)-(3-hydroxyhexahydropyrrolo[3,2-b]pyrrole-1-yl)phenylmethanone (136) as a colourless oil (309 mg) which was used without further purification. HPLC-MS 233.1 [M+H]⁺, 465.1 [2M+H]⁺.

### Preparation of (3aR,6aS)[(1S)-1-((6S)-4-benzoyl-hydroxyhexa hydropyrrolo[3,2-b]pyrrole-1-carbonyl)-3-methylbutyl]carbamic acid benzyl ester (137)

Cbz-Leu-F (350 mg, 1.31 mmol) was dissolved in dimethylformamide (5 ml) then added to (3S,3aS,6aR)-(3-hydroxyhexahydropyrrolo[3,2-b]pyrrole-1-yl)phenyl methanone (136) (304 mg, 1.24 mmol, prepared as above) under an atmosphere of argon. The solution was stirred for 1.25 hours then the solvents removed in vacuo. The residue was purified by flash chromatography over silica eluting with ethyl acetate:heptane mixtures 0:100 to 80:20 to give (3aR,6aS)-[(1S)-1-((6S)-4 benzoyl-6-hydroxyhexahydropyrrolo[3,2-b]pyrrole-1-carbonyl)-3-methylbutyl] carbamic acid benzyl ester (137) as a white solid (402 mg, 68%). TLC (Single spot, R_{f}=0.10, EtOAc:heptane 65:35), analytical HPLC Rₜ=16.803 min; HPLC-MS 480.2 [M+H]⁺, 981.3 [2M+Na]⁺; HRMS C₂₇H₃₃N₃O₅Na req. 502.2318, fnd. 502.2311 (−1.44 ppm).

### Alternative preparation of (3aR,6aS)-[(1S)-1-(6S)-4-(benzoyl-6-hydroxyhexa hydropyrrolo[3,2-b]pyrrole-1-carbonyl)-3-methylbutyl]carbamic acid benzyl ester (137)

Dimethylformamide (1 ml) was added to a mixture of (3S,3aS,6aR)-(3-hydroxyhexahydropyrrolo[3,2-b]pyrrole-1-yl)phenylmethanone (136) (24 mg, 0.087 mmol, prepared as above) and Cbz-Leu-OSuc (32 mg, 0.088 mmol) under an atmosphere of argon. The solution was stirred for 20 hours then the solvents removed in vacuo to obtain a residue which was dissolved in dichloromethane (20 nm) then washed with water (10 ml), dried (Na₂SO₄) and the solvents removed in vacuo. The residue was purified by flash chromatography over silica eluting with ethyl acetate:heptane mixtures 30:70 to 80:20 to give (3aR,6aS)-[(1S)-1-((6S)-4-benzoyl-6-hydroxyhexahydropyrrolo[3,2-b]pyrrole-1-carbonyl)-3-methyl butyl]carbamic acid benzyl ester (137) as a white solid (25 mg, 60%). TLC (Single spot, R_{f}=0.10, EtOAc:heptane 65:35), analytical HPLC Rₜ=17.301 min; HPLC-MS 480.2 [M+H]⁺, 981.3 [2M+Na]⁺; C₂₇H₃₃N₃O₅.0.2EtOAc req. (fnd.) % C 67.20 (67.03), % H 7.02 (7.16), % N 8.45 (8.27); d_{H} (500 MHz, CDCl₃) mixture of rotamers, tentative assignment of proton 1.2-2.4 (11H, m, 2×Leu δCH₃, Leu βCH₂, Leu γCH, BzNCHCH₂), 3.3-4.0 (4H, m, BzNCH₂, CbzLeuNCH₂), 4.2-5.0 (4H, BzNCH, CbzLeuNCH, CHOH, Leu αCH), 5.0-5.1 (2H, OCH₂Ph), 5.4 (1H, d, J=8.3 Hz, NH), 7.4-7.6 (10H, aromatic); d_{C} (125 MHz, CDCl₃) 21.73, 21.89 and 23.22, 23.36 (2× Leu δCH₃), 24.59, 24.67 (Leu γCH), 31.86 (BzNCHCH₂), 42.02, 42.22 (Leu PCH₂), 46.52 (CbzLeuNCH₂), 50.94, 51.02 (Leu αCH), 56.58 (BzNCH₂), 59.72 (BzNCH), 67.00 (OCH₂Ph), 67.98 (CbzLeuNCH), 75.25 (CHOH), 127.34, 128.02, 128.18, 128.28, 128.36, 128.52, 130.34 (aromatic CH), 136.09, 136.18 (aromatic quaternary), 156.18 (NHC═O), 170.08 (PhC═O), 172.32 (CH₂NC═O).

### Preparation of (2S,3aR,6aS)-2-amino-1-((6S)-4-benzoyl-6-hydroxyhexahydro pyrrolo[3,2-b]pyrrole-1-yl)-4-methylpentan-1-one (138)

Ethanol (15 ml) was added cautiously to a stirred mixture of (3aR,6aS)-[(1S)-1-((6S)-4-benzoyl-6-hydroxyhexahydropyrrolo[3,2-b]pyrrole-1-carbonyl)-3-methylbutyl] carbamic acid benzyl ester (137) (370 mg, 0.77 mmol) and 10% palladium on charcoal (50 mg) at 0° C. under an atmosphere of argon. The argon was replaced by an atmosphere of hydrogen then stirring continued at ambient temperature for 1.75 hours. The hydrogen was replaced by argon then the mixture was cooled to 0° C. before adding a further portion of 10% palladium on charcoal (20 mg). The argon was replaced by hydrogen then stirring continued at ambient temperature for 5.25 hours. The hydrogen was replaced by argon then the mixture was cooled to 0° C. before adding a further portion of 10% palladium on charcoal (20 mg). The argon was replaced by hydrogen then stirring continued at ambient temperature for 14 hours. The hydrogen was replaced by argon then the mixture was cooled to 0° C. before adding a further portion of 10% palladium on charcoal (10 mg). The argon was replaced by hydrogen then stirring continued at ambient temperature for 2 hours. The hydrogen was replaced by argon then the mixture was diluted with ethanol (60 ml) and filtered through celite. The filter cake was washed with ethanol (40 ml) then the filtrate concentrated in vacuo. The residue was azeotroped with ethyl acetate (35 ml) to obtain (2S,3aR,6aS)-2-amino-1-((6S)-4-benzoyl-6-hydroxyhexahydropyrrolo[3,2-b]pyrrole-1-yl)-methylpentan-1-one (138) as an oily white solid (270 mg), which was used without further purification. HPLC-MS 346.2 [M+H]⁺, 713.3 [2M+Na]⁺.

### Preparation of (3aR,6aS)-N-[(1-(1S)-1-((6S)-4-benzoyl-6-hydroxyhexahydro-pyrrolo[3,2-b]pyrrole-1-carbonyl)-3-methylbutyl-4-tert-butyl-benzamide (139)

4-Methylmorpholine (0.17 ml, 1.55 mmol) was added to a solution of HBTU (293 mg, 0.77 mmol), 1-hydroxybenzotriazole monohydrate (118 mg, 0.77 mmol) and 4-(tert-butyl)benzoic acid (138 mg, 0.77 mmol) in dimethylformamide (7.5 ml). The solution was stood for 5 minutes then added to (2S,3aR,6aS)-2-amino-1-((6S)-4-benzoyl-6-hydroxyhexahydropyrrolo[3,2-b]pyrrole-1-yl]-4-methylpentan-1-one (138) (prepared as above, 0.77 mmol). The mixture was stirred at ambient temperature for 1 hour 5 minutes then the solvents removed in vacuo (water bath temperature<28° C.). The residue was dissolved in dichloromethane (75 ml) then washed with pH 3 hydrochloric acid (60 ml), saturated aqueous sodium hydrogen carbonate solution (60 ml) and brine (60 ml), dried (Na₂SO₄) and the solvents removed in vacuo. The residue (512 mg) was purified by flash chromatography over silica eluting with ethyl acetate:heptane mixtures 0:100 to 85:15 to give (3aR,6aS)-N-[(1S)-1-((6S)-4-benzoyl-6-hydroxyhexahydro-pyrrolo[3,2-b]pyrrole-1-carbonyl)-3-methylbutyl]-4-tert-butyl-benzamide (139) as a white solid (263 mg, 68%). TLC (Single spot, R_{f}=0.15, EtOAc:heptane 9:1), analytical HPLC Rₜ=19.340 min; HPLC-MS 506.2[M+H]⁺; C₃₀H₃₉N₃O₄.0.5EtOAc req. (fnd.) % C 69.97 (69.86), % H 7.89 (7.87), % N 7.65 (7.88); HRMS C₃₀H₃₉N₃O₄Na req. 528.2838, fnd. 528.2818 (−3.89 ppm).

### Preparation of (3aR,6aS)-N-[(1S)-1-(4-benzoyl-6-oxo-hexahydropyrrolo[3,2-b]pyrrole-1-carbonyl)-3-methylbutyl]-4-tert-butylbenzamide (EXAMPLE 1)

A solution of (3aR,6aS)-N-[(1S)-1-((6S)-4-benzoyl-6-hydroxyhexahydro-pyrrolo[3,2-b]pyrrole-1-carbonyl)-3-methylbutyl]-4-tert-butyl-benzamide (139) (174 mg, 0.345 mmol) in dichloromethane (10 ml) was added to Dess-Martin periodinane (292 mg, 0.689 mmol) under an atmosphere of argon whilst stirring over 2.5 minutes. The mixture was stirred for 3 minutes then trifluoroacetic acid (53 μl, 0.689 mmol) was added. The mixture was stirred for 14 hours then solvents removed in vacuo. The residue was purified by flash chromatography over silica eluting with ethyl acetate:heptane mixtures 0:100 to 55:45 to give (3aR,6aS)-N-[(1S)-1-(4-benzoyl-6-oxo-hexahydropyrrolo[3,2-b]pyrrole-1-carbonyl)-3-methylbutyl]-4-tert-butylbenzamide (EXAMPLE 1) as a white solid (128 mg, 74%). TLC (Single spot, R_{f}=0.20, EtOAc:heptane 9:1), analytical HPLC broad peak Rₜ=19.2-20.6 min; HPLC-MS single broad UV peak, 504.1 [M+H]⁺.

### Preparation of (3aR,6aS)-[(1S)-1-(4-benzoyl-6-oxo-hexahydropyrrolo[3,2-b]pyrrole-1-carbonyl)-3-methylbutyl]carbamic acid benzyl ester (EXAMPLE 361)

A solution of (3aR,6aS)-[(1S)-1-((6S)-4-benzoyl-6-hydroxyhexa hydropyrrolo[3,2-b]pyrrole-1-carbonyl)-3-methylbutyl]carbamic acid benzyl ester (137) (15.0 mg, 0.031 mmol) in dichloromethane (0.75 ml) was added dropwise to Dess-Martin periodinane (26.6 mg, 0.063 mmol) under an atmosphere of argon whilst stirring over 1 minute. The solution was stirred for 4.5 hours then purified by flash chromatography over silica eluting with ethyl acetate:heptane mixtures 0:100 to 80:20 to give (3aR,6aS)-[(1S)-1-(4-benzoyl-6-oxo-hexahydropyrrolo[3,2-b]pyrrole-1-carbonyl)-3-methylbutyl]carbamic acid benzyl ester (EXAMPLE 361) as a white solid (8.8 mg, 58%). TLC (Single spot, R_{f}=0.35, EtOAc:heptane 9:1), analytical HPLC broad peak Rₜ=17.7-19.5 min; HPLC-MS single broad UV peak, 478.1 [M+H]⁺, 977.2 [2M+Na]⁺; HRMS C₂₇H₃₁N₃O₅Na req. 500.2161, fnd. 500.2168 (1.26 ppm).

Alternatively, the general synthetic strategy detailed in Scheme 28 involves construction of an extended compound prior to intramolecular ring closure to the 5,5-cis bicycle as the penultimate step. As detailed in Scheme 20, the building block (R)-2-(2-azidoethyl)-2,5 dihydropyrrole-1-carboxylic acid tert-butyl ester (76) may be reduced to the primary amine, which in Scheme 28 is directly acylated with a protected aminoacid. Following epoxidation, then conversion of the aminoacid protecting group to a suitable capping group, with all of the potency and specificity components now in place, ring-closure and oxidation provides the final inhibitor compound. The utility of such synthetic flexibility is detailed in an alternativesynthesis of EXAMPLE 1, through Scheme 28.

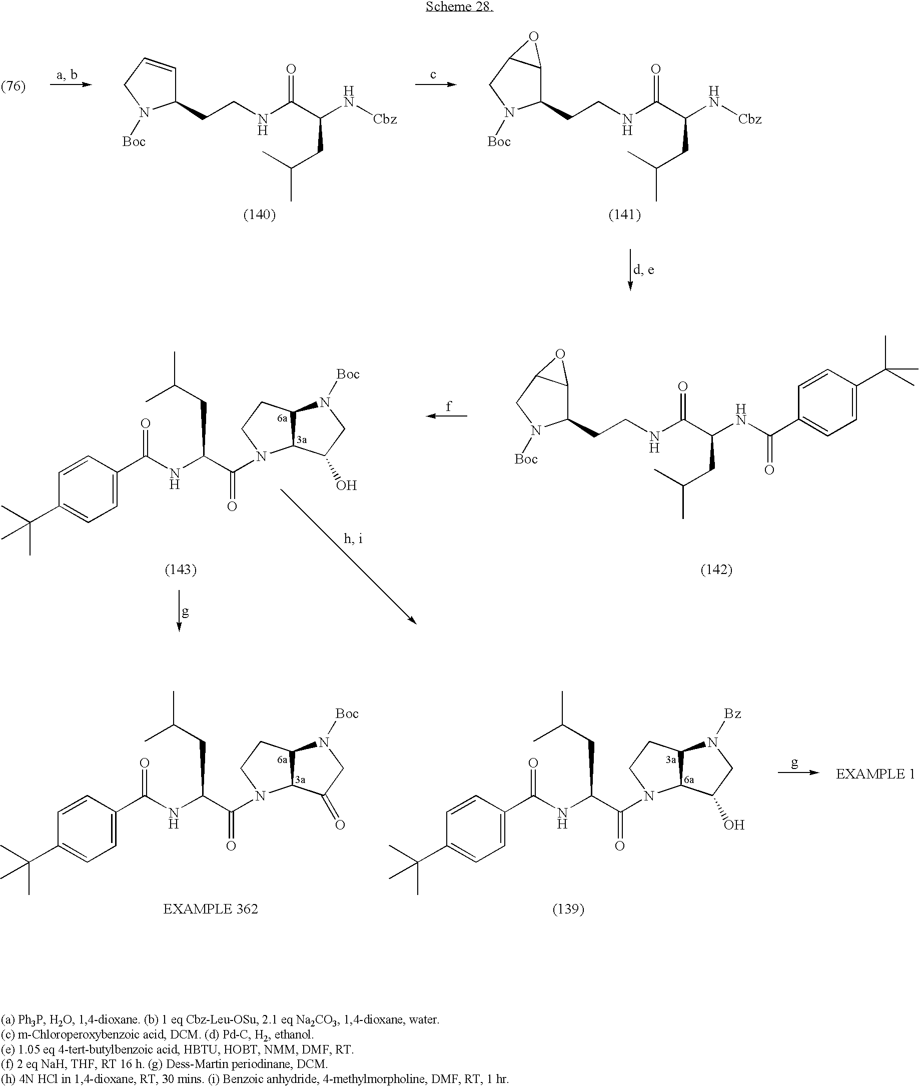

### Preparation of (2R)-2-[2-((2S)-2-benzyloxycarbonylamino-4-methylpentanoyl amino)ethyl]-2,5-dihydro pyrrole-1-carboxylic acid tert-butyl ester (140)

(R)-2-(2-Aminoethyl)-2,5-dihydropyrrole-1-carboxylic acid tert-butyl ester (see preparation of (77) above, ˜0.63 mmol) was dissolved in 1,4-dioxane (10 ml) with stirring, ice-cooled and a solution of sodium carbonate (0.14 g, 1.32 mmol) in water (10 ml) was added. Cbz-L-Leu-OSu (0.251 g, 0.693 mmol) in 1,4-dioxane (10 ml) was added dropwise over 30 minutes, then the ice bath removed and the mixture stirred for a further 30 minutes. Water (100 ml) was then added and the aqueous phase extracted with dichloromethane (2×100 ml). The combined organic layers were dried (Na₂SO₄), filtered and reduced in vacuo to leave a clear gum (0.54 g). Flash chromatography over silica, eluting with ethyl acetate:heptane mixtures gave (2R)-2-[2-((2S)-2-benzyloxycarbonylamino-4-methylpentanoylamino)ethyl]-2,5-dihydro pyrrole-1-carboxylic acid tert-butyl ester (140) (0.21 g, 72%) as a clear oil. TLC (R_{f}=0.30, EtOAc:heptane 1:1), analytical HPLC single main peak, Rₜ=20.326 min., HPLC-MS 360.1 [M+2H−Boc]⁺, 404.1 [M+2H−Bu]⁺, 460.2 [M+H]⁺, 482.1 [M+Na]⁺, 941.2 [2M+Na]⁺; Elemental analysis C₂₅H₃₇N₃O₅ req. (fnd.) % C 65.34 (65:14), % H 8.11 (8.19), % N 9.14 (9.07); HRMS C₂₅H₃₇N₃O₅Na req. 482.2631, fnd. 482.2620 (−2.33 ppm).

### Preparation of (2R)-2-[2-((2S)-2-benzyloxycarbonylamino-4-methylpentanoyl amino)ethyl]-6-oxa-3-azabicyclo[3.1.0]hexane-3-carboxylic acid tert-butyl ester (141)

(2R)-2-[2-((2S)-2-Benzyloxycarbonylamino-4-methylpentanoylamino)ethyl]-2,5-di hydropyrrole-1-carboxylic acid tert-butyl ester (140) (0.20 g, 0.435 mmol) was dissolved in dichloromethane (5 ml) with stirring then meta-chloroperoxybenzoic acid (65% reagent, 1.15 g, 4.35 mmol) added. The mixture was stirred at ambient temperature under argon for 16 hours. Dichloromethane (100 ml) was added and the organic phase washed with 10% w/v aqueous sodium hydroxide solution (2×100 ml), then dried (Na₂SO₄), filtered and reduced in vacuo to leave an oily solid (0.19 g). Flash chromatography over silica, eluting with ethyl acetate:heptane mixtures gave (2R)-2-[2-((2S)-2-benzyloxycarbonylamino-4-methylpentanoyl amino)ethyl]-6-oxa-3-azabicyclo[3.1.0]hexane-3-carboxlic acid tert-butyl ester (141) (0.19 g, 92%) as an opaque gum. TLC (R_{f}=0.35 (major) and 0.42 (minor) (mixture of anti and syn epoxides), EtOAc:heptane 3:1), analytical HPLC single main peak, Rₜ=19.21 min., HPLC-MS 376.1 [M+2H−Boc]⁺, 420.1 [M+2H−Bu]⁺, 476.1 [M+H]⁺, 498.1 [M+Na]⁺, 973.2 [2M+Na]⁺; Elemental analysis C₂₅H₃₇N₃O₆ req. (fnd.) % C 63.14 (63.11), % H 7.84 (7.96), % N 8.84 (8.80); HRMS C₂₅H₃₇N₃O₆Na req. 498.2580, fnd. 498.2602 (4.34 ppm).

### Preparation of (2R)-2-{2-[(2S)-2-(4-tert-butylbenzoylamino)-4-methyl pentanoylamino]ethyl}-6-oxa-3-aza-bicyclo[3.1.0]hexane-3-carboxylic acid tert-butyl ester (142)

(i) (2R)-2-[2-((2S)-2-Benzyloxycarbonylamino-4-methylpentanoylamino)ethyl]-6-oxa-3-azabicyclo [3.1.0]hexane-3-carboxlic acid tert-butyl ester (141) (0.17 g, 0.357 mmol) was dissolved in ethanol (5 ml), cooled to 0° C. and 10% palladium on charcoal (0.034 g) added. The mixture was stirred, then evacuated and flushed with hydrogen. The mixture was allowed to warm to ambient temperature, stirred for 45 minutes then filtered through celite. The filter cake was washed with ethanol (3×10 ml) and the combined organic layers reduced in vacuo to provide the crude free amine, which was used without further purification. HPLC-MS 342.2 [M+H]⁺, 683.3 [2M+H]⁺, 705.3 [2M+Na]⁺.

(ii) The crude free amine was dissolved in anhydrous dimethylformamide (3 ml) with stirring and 2-(1H-benzotriazole-1-yl)-1,1,3,3-tetramethyluroniumhexafluoro phosphate (HBTU, 0.136 g, 0.357 mmol) and 1-hydroxybenzotriazole monohydrate (HOBT, 0.0548 g, 0.357 mmol) added. 4-Methylmorpholine (78.6 μl, 0.715 mmol) was added and the mixture stirred for 1.5 hours, then reduced in vacuo. The residue was dissolved in dichloromethane (50 ml) and washed with pH 3 hydrochloric acid (50 ml), saturated sodium hydrogen carbonate solution (50 ml) and brine (50 ml). The organic phase was dried ((Na₂SO₄), filtered and reduced in vacuo to leave a pale yellow gum (0.19 g). Flash chromatography over silica, eluting with ethyl acetate:heptane mixtures gave (2R)-2-{2-[(2S)-2-(4-tert-butylbenzoylamino)-4-methylpentanoylamino]ethyl}-6-oxa-3-aza-bicyclo[3.1.0] hexane-3-carboxylic acid tert-butyl ester (142) (0.08 g, 45%) as a white crystalline solid. TLC (R_{f}=0.26, EtOAc:heptane 3:1), analytical HPLC single main peak, Rₜ=21.195 min. HPLC-MS 446.2 [M+2H−Bu]⁺, 502.3 [M+H]⁺, 524.2 [M+Na]⁺; Elemental analysis C₂₈H₄₃N₃O₅ req. (fnd.) % C 67.04 (67.07), % H 8.64 (8.96), % N 8.38 (7.87); HRMS C₂₈H₄₃N₃O₅Na req. 524.3100, fnd. 524.3086 (−2.81 ppm).

### Preparation of (3S,3aS,6aR)-4-[(2S)-2-(4-tert-butylbenzoylamino)-4-methylpentanoyl]-3-hydroxyhexahydropyrrolo[3,2-b]pyrrole-1-carboxylic acid tert-butyl ester (143)

(2R)-2-{2-[(2S)-2-(4-tert-Butylbenzoylamino)-4-methylpentanoylamino]ethyl}-6-oxa-3-aza-bicyclo [3.1.0]hexane-3-carboxylic acid tert-butyl ester (142) (0.06 g, 0.12 mmol) was dissolved in tetrahydrofuran (3 ml) with stirring under nitrogen and ice-cooled. Sodium hydride (60% dispersion in oil, 0.010 g, 0.25 mmol) was added over 1 minute and the mixture stirred at ambient temperature for 16 hours. Water (10 ml) was added, then saturated aqueous ammonium chloride solution (5 ml) and the product extracted into ethyl acetate (2×25 ml). The combined organic layers were dried (Na₂SO₄), filtered and reduced in vacuo to leave a clear film (0.06 g). Flash chromatography over silica, eluting with ethyl acetate:heptane mixtures gave (3S,3aS,6aR)-4-[(2S)-2-(4-tert-butylbenzoylamino)-4-methylpentanoyl]-3-hydroxyhexa hydropyrrolo[3,2-b]pyrrole-1-carboxylic acid tert-butyl ester (143) (0.021 g, 35%) as a white solid. TLC (R_{f}=0.40, EtOAc:heptane 2:1), HPLC-MS 502.3 [M+H]⁺, 524.2 [M+Na]⁺.

A second product fraction contaminated by starting epoxide (˜25% by WV analysis) was obtained as a white solid (0.0239 g).

### Preparation of (3aS,6aR)-4-[(2S)-2-(4-tert-butylbenzoylamino)-4-methyl pentanoyl]-3-oxo-hexahydropyrrolo[3,2-b]pyrrole-1-carboxylic acid tert-butyl ester (EXAMPLE 362)

(3S,3aS,6aR)-4-[(2S)-2-(4-tert-Butylbenzoylamino)-4-methylpentanoyl]-3-hydroxy hexahydropyrrolo[3,2-b]pyrrole-1-carboxylic acid tert-butyl ester (143) (0.021 g, 0.042 mmol) was dissolved in dichloromethane (2 ml) with stirring under argon. Dess-Martin periodinane (0.0373 g, 0.088 mmol) was added and the mixture stirred for 16 hours. The mixture was reduced in vacuo and the residue purified by flash chromatography over silica, eluting with ethyl acetate:heptane mixtures to give (3aS,6aR)-4-[(2S)-2-(4-tert-butylbenzoylamino)-4-methylpentanoyl]-3-oxo-hexahydro pyrrolo[3,2-b]pyrrole-1-carboxylic acid tert-butyl ester (EXAMPLE 362) (0.015 g, 71%) as an off-white gum. TLC (R_{f}=0.53, EtOAc:heptane 2:1), analytical HPLC single broad main peak, Rₜ=20.6-22.5 min., HPLC-MS 500.1 [M+H]⁺. HRMS C₂₈H₄₁N₃O₅Na req. 522.2944, fnd. 522.2952 (1.49 ppm); δ_{H} (500 z, CDCl₃) 0.85-1.03 (7H, m, 2× Leu δCH₃+Leu γCH), 1.32 (9H, s, (CH₃)₃CPh), 1.47 (9H, s, (CH₃)₃COCO), 1.6-1.8 (2H, m, Leu βCH₂), 2.03-2.15/2.33-2.45 (2H, b, BocNCHCH₂), 3.50-3.60 (1H, m, LeuNCH₂), 3.75-3.82 (1H, m, BocNCH₂), 3.93-4.02 (1H, m, BocNCH₂), 4.02-4.08 (1H, m, LeuNCH₂), 4.58-4.80 (1H, b, BocNCH or LeuNCH), 4.96-4.98 (1H, b, BocNCH or LeuNCR), 5.0-5.06/5.25-5.30 (1H, bm, Leu αCH), 6.83-6.93 (1H, b, LeuNH), 7.42-7.45 (2H, d, J=8.5 Hz, (CH₃)₃C—C—CH═CH), 7.72-7.75 (2H, d, J=8.5 Hz, (CH₃)₃C—C—CH═CH); δ_{C} (125 MHz, CDCl₃) 22.27, 23.48 (2×Leu δCH₃), 24.78 (Leu γCH), 28.32 ((CH₃)₃COCO), 29.64, 31.82 (BocNCHCH₂), 31.09 ((CH₃)₃CPh), 34.88 ((CH₃)₃CPh), 42.50 (Leu βCH₂), 45.95 (LeuNCH₂), 49.09, 49.67 (Leu αCH), 52.37 (BocNCH₂), 56.82 (BocNCH), 62.92 (LeuNCH), 81.16 ((CH₃)₃COCO), 125.34, 125.44 ((CH₃)₃C—C—CH═CH), 126.89, 126.95 ((CH₃)₃C—C—CH═CH), 130.83 (quatemnary aromatics), 155.26 ((CH₃)₃COCO), 167.01 (CH₃)₃CPhCO), 172.28 ((Leu C═O).

### Preparation of (3aR,6aS)-N-[(1S)-1-((6S)-4-benzoyl-6-hydroxyhexahydro pyrrolo[3,2-b]pyrrole-1-carbonyl)-3-methylbuty]-4-tert-butylbenzamide (139)

(i) (3S,3aS,6aR)-4-[(2S)-2-(4-tert-Butylbenzoylamino)-4-methylpentanoyl]-3-hydroxyhexahydro pyrrolo[3,2-b]pyrrole-1-carboxylic acid tert-butyl ester (143) (0.023 g, 0.046 mmol) was dissolved in 4.0M HCl in 1,4-dioxane (2 ml, 8 mmol) with stirring. After 45 minutes the solvents were removed in vacuo and the residue triturated then evaporated from diethyl ether (3×3 ml) to leave (3aR,6aS)-4-tert-butyl-N-[(1S)-1-((6S)-6-hydroxyhexahydropyrrolo[3,2-b]pyrrole-1-carbonyl)-3-methylbutyl]benzamide hydrochloride as a white solid which was used without further purification; HPLC-MS 402.2 [M+H]⁺, 424.2 [M+Na]⁺, 803.4 [2M+H]⁺, 825.4 [2M+Na]⁺.

(ii) (3aR,6aS)-4-tert-Butyl-N-[(1S)-1-((6S)-6-hydroxyhexahydropyrrolo[3,2-b]pyrrole-1-carbonyl)-3-methylbutyl]benzamide hydrochloride (prepared as above, ˜0.048 mmol) was dissolved in dimethylformamide (2 ml) with stirring, then benzoic anhydride (0.0114 g, 0.05 mmol) added followed by 4-methylmorpholine (11.1 μl, 0.0102 g, 0.101 mmol). After 1 hour, ethyl acetate (25 ml) was added and the organics washed with saturated aqueous sodium hydrogen carbonate solution (25 ml) solution, pH 3 hydrochloric acid (25 ml), and brine (25 ml). The organics were dried (Na₂SO₄), filtered and reduced in vacuo to a colourless film (3aR,6aS)-N-[(1S)-1-((6S)-4-benzoyl-6-hydroxyhexahydropyrrolo[3,2-b]pyrrole-1-carbonyl)-3-methylbuty]-4-tert-butylbenzamide (139) (0.028 g). TLC (R_{f}=0.16, EtOAc:heptane 4:1), analytical HPLC single broad main peak, Rₜ=19.15 min., HPLC-MS 506.2 [M+H]⁺, 528.2 [M+Na]⁺.

Oxidation to EXAMPLE 1 is as detailed in Scheme 27.

### EXAMPLE A

### Assays for Cysteine Protease Activity

The compounds of this invention may be tested in one of a number of literature based biochemical assays that are designed to elucidate the characteristics of compound inhibition. The data from these types of assays enables compound potency and the rates of reaction to be measured and quantified. This information, either alone or in combination with other information, would allow the amount of compound required to produce a given pharmacological effect to be determined.

General Materials and Methods

Unless otherwise stated, all general chemicals and biochemicals were purchased from either the Sigma Chemical Company, Poole, Dorset, U.K or from Fisher Scientific UK, Loughborough, Leicestershire, U.K. Absorbance assays were carried out in flat-bottomed 96-well plates (Spectra; Greiner Bio-One Ltd., Stonehouse, Gloucestershire, U.K.) using a SpectraMax PLUS384 plate reader (Molecular Devices, Crawley, U.K.). Fluorescence high throughput assays were carried out in either 384-well microtitre plates (Corning Costar 3705 plates, Fisher Scientific) or 96-well ‘U’ bottomed Microfluor W1 microtitre plates (Thermo Labsystems, Ashford, Middlesex, U.K.). Fluorescence assays were monitored using a SpectraMax Gemini fluorescence plate reader (Molecular Devices). For substrates employing either a 7-amino-4-methylcoumarin (AMC) or a 7-amino-4-trifluoromethylcoumarin (AFC) fluorophore, assays were monitored at an excitation wavelength of 365 nm and an emission wavelength of 450 nm and the fluorescence plate reader calibrated with AMC. For substrates employing a 3-amino-benzoyl (Abz) fluorophore, assays were monitored at an excitation wavelength of 310 nm and an emission wavelength of 445 nm; the fluorescence plate reader calibrated with 3-amino-benzamide (Fluka). Unless otherwise indicated, all the peptidase substrates were purchased from Bachem UK, St Helens, Merseyside, UK. Substrates utilizing fluorescence resonance energy transfer methodology (i.e. FRET-based substrates) were synthesized at Incenta Limited using published methods (Atherton & Sheppard, Solid Phase Peptide Synthesis, IRL Press, Oxford, U.K, 1989) and employed Abz (2-aminobenzoyl) as the fluorescence donor and 3-nitro-tyrosine [Tyr(NO₂)] as the fluorescence quencher (Meldal, M. and Breddam, K., Anal. Biochem, 195, 141-147, 1991). Hydroxyethylpiperazine ethanesulfonate (HEPES), tris-hydroxylmethyl aminomethane (tris) base, bis-tris-propane and all the biological detergents (e.g. CHAPS, zwittergents, etc.) were purchased from CN Biosciences UK, Beeston, Nottinghamshire, U.K. Glycerol was purchased from Amersham Pharmacia Biotech, Little Chalfont, Buckinghamshire, U.K. Stock solutions of substrate or inhibitor were made up to 10 mM in 100% dimethylsulfoxide (DMSO) (Rathburns, Glasgow, U.K.) and diluted as appropriately required. In all cases the DMSO concentration in the assays was maintained at less than 1% (vol./vol.).

Assay protocols were based on literature precedent (Table 4; Barrett, A. J., Rawlings, N. D. and Woessner, J. F., 1998, Handbook of Proteolytic Enzymes, Academic Press, London and references therein) and modified as required to suit local assay protocols. Enzyme was added as required to initiate the reaction and the activity, as judged by the change in fluorescence upon conversion of substrate to product, was monitored over time. All assays were carried out at 25±1° C.

TABLE 4
The enzyme assays described herein were carried out according to
literature precedents.

| Enzyme | Buffer | Substrate | Reference |
|---|---|---|---|
| Cathepsin B | I | Z-Phe-Arg-AMC | a, b |
| Cathepsin H | II | Bz-Phe-Val-Arg-AMC | a, b |
| Cathepsin L | I | Ac-Phe-Arg-AMC | b, c |
| Cathepsin S | I | Boc-Val-Leu-Lys-AMC | c, d |
| Caspase 1 | III | Ac-Leu-Glu-His-Asp-AMC | e |
| Caspase 2 | III | Z-Val-Asp-Val-Ala-Asp-AFC | f |
| Caspase 3 | III | Ac-Asp-Glu-Val-Asp-AMC | g, h |
| Caspase 4 | III | Suc-Tyr-Val-Ala-Asp-AMC | f |
| Caspase 5 | III | Ac-Leu-Glu-His-Asp-AMC | |
| Caspase 6 | III | Ac-Val-Glu-Ile-Asp-AMC | i, j, k |
| Caspase 7 | III | Ac-Asp-Glu-Val-Asp-AMC | |
| Caspase 8 | III | Ac-Ile-Glu-Thr-Asp-AMC | l |
| Caspase 9 | III | Ac-Leu-Glu-His-Asp-AMC | |
| Caspase 10 | III | Ac-Ile-Glu-Thr-Asp-AMC | |
| Cruzipain | IV | D-Val-Leu-Lys-AMC | m, n |
| CPB2.8ΔCTE | XI | Pro-Phe-Arg-AMC | q |
| S. Aureus | I | Abz-Ile-Ala-Ala-Pro- | o |
| Extracellular | | Tyr(NO₂)-Glu-NH₂ | |
| cysteine | | | |
| peptidase | | | |
| Clostripain | | Z-Gly-Gly-Arg-AMC | p |
| FMDV LP | V | Abz-Arg-Lys-Leu-Lys-Gly- | r |
| | | Ala-Gly-Ser-Tyr(NO₂)-Glu- | |
| | | NH₂ | |
| Trypsin | VI | Z-Gly-Gly-Arg-AMC | s |
| Calpain μ | VII | Abz-Ala-Asn-Leu-Gly-Arg-Pro- | t |
| | | Ala-Leu-Tyr(NO₂)-Asp-NH₂ | |
| Calpain m | VIII | Abz-Lys-Leu-Cys(Bzl)-Phe-Ser- | t |
| | | Lys-Gln-Tyr(NO₂)-Asp-NH₂ | |
| Cathepsin K | IX | Z-Phe-Arg-AMC | u |
| Cathepsin X | X | | v, w |

I: 10 mM BTP, pH 6.5 containing 1 mM EDTA, 5 mM 2-mercaptoethanol and 1 mM CaCl₂
II: 10 mM BTP, pH 6.5 containing 1 mM EDTA, 142 mM NaCl, 1 mM DTT, 1 mM CaCl₂, 0.035 mM Zwittergent 3-16
III: 50 mM HEPES pH 7.2, 10% Glycerol, 0.1% CHAPS, 142 mM NaCl, 1 mM EDTA, 5 mM DTT
IV: 100 mM sodium phosphate, pH 6.75 containing 1 mM EDTA and 10 mM L-cysteine
V: 50 mM tris.acetate, pH 8.4 containing 1 mM EDTA, 10 mM L-cysteine and 0.25% (w/v) CHAPS
VI: 10 mM HEPES, pH 8.0 containing 5 mM CaCl₂
VII: 10 mM HEPES, pH 7.5 containing 2 mM 2-mercaptoethanol and 100 μM CaCl₂
VIII: 10 mM HEPES, pH 7.5 containing 2 mM 2-mercaptoethanol and 200 μM CaCl₂
IX: 100 mM sodium acetate; pH 5.5 containing 10 mM L-cysteine and 1 mM EDTA
X: 100 mM sodium acetate; pH 5.5 containing 10 mM L-cysteine; 0.05% (w/v) Brij 35 and 1 mM EDTA
XI: 100 mM sodium acetate; pH 5.5 containing 10 mM L-cysteine; 142 mM sodium chloride and 1 mM EDTA
a Barrett, A. J., Biochem. J., 187, 909-912, 1980
b Barret, A. J. and Kirschke, H., Methods Enzymol., 80, 535-561, 1981
c Quibell, M. and Taylor, S., WO0069855, 2000
d Bromme, D., Steinert, ., Freibe, S., Fittkau, S., Wiederanders, B., and Kirschke, H., Biochem. J., 264, 475-481, 1989
e Rano T. A., et. al., Chem. Biol., 4, 149, 1997
f Talanian, R. V., et. al., J. Biol. Chem., 272, 9677, 1997
g Lazebnik, Y. A., Kaufmann, S. H., Desnoyers, S., Poirer, G. G. and Earnshaw, W. C., Nature., 371, 768-774, 1994
h Han, Z., et. al., J. Biol. Chem., 272, 13432, 1997
i Takahashi, A., et. al., PNAS, 93, 8395, 1996
j Martins, L. M., et. al., J. Biol. Chem., 272, 7421, 1997
k Nagata, S., Cell., 88, 355, 1997
l Harris, J. L., et. al., J. Biol. Chem., 273, 27364, 1998
m Cazzulo, J. J., Cazzulo Franke, M. C., Martinez, J. and Franke de Cazzulo, B. M., Biochim. Biophys. Acta., 1037, 186-191, 1990
n Cazzulo, J. J., Bravo, M., Raimondi, A., Engstrom, U., Lindeberg, G. and Hellman, U., Cell Mol. Biol., 42, 691-696, 1996
o Potempa, J., Dubin, A., Korzus, G. and Travis, J., Biochem. J., 263, 2664-2667, 1988
p Kembhavi, A. A., Buttle, D. J., Rauber, P. and Barrett, A. J., FEBS Lett., 283, 277-280, 1991
q Alves, L. C., et. al., Mol. Biochem. Parasitol, 116, 1-9, 2001.
r Guarné, et.al., J. Mol. Biol., 302, 1227-1240, 2000.
s Halfon and Craik, (Barret, Rawlings and Woessner, eds.), in Handbook of Proteolytic Enzymes, Academic Press, London, 12-21, 1998.
t Sasaki, et. al., (1984), J. Biol. Chem., 259, 12489-12494, 1984.
u Bossard, M. J., et. al., , J. Biol. Chem., 21, 12517-12524, 1996
v Santamaria, I., et. al., J. Biol. Chem., 273, 16816-16823, 1998
w Klemencic, J, et al., Eur.J.Biochem., 267, 5404-5412, 2000

Trypanosoma cruzi Cruzipain Peptidase Activity Assays

Wild-type cruzipain, derived from Trypanosoma cruzi Dm28 epimastigotes, was obtained from Dr. Julio Scharfstein (Instituto de Biofisica Carlos Chagas Filho, Universidade Federal do Rio de Janeiro, Rio de Janeiro, Brazil). Activity assays were carried out in 100 mM sodium phosphate, pH 6.75 containing 1 mM EDTA and 10 mM L-cysteine using 2.5 nM enzyme. Ac-Phe-Arg-AMC (K_{M}^{app˜} 12 μM) and D-Val-leu-Lys-AMC (K_{M}^{app˜} 4 μM) were used as the substrates. Routinely, Ac-FR-AMC was used at a concentration equivalent to K_{M}^{app} and D-Val-Leu-Lys-AMC was used at a concentration of 25 μM. The rate of conversion of substrate to product was derived from the slope of the increase in fluorescence monitored continuously over time.

Leishmania mexicana Cysteine Protease B (CPB) Peptidase Activity Assays

Wild-type recombinant CPB without the C-terminal extention (i.e. CPB2.8ΔCTE; Sanderson, S. J., et. al., Biochem. J., 347, 383-388, 2000) was obtained from Dr. Jeremy Mottram (Wellcome Centre for Molecular Parasitology, The Anderson College, University of Glasgow, Glasgow, U.K.). Activity assays were carried out in 100 mM sodium acetate; pH 5.5 containing 1 mM EDTA; 200 mM NaCl and 10 mM DTT (Alves, L. C., et. al., Mol. Biochem. Parasitol, 116, 1-9, 2001) using 0.25 nM enzyme. Pro-Phe-Arg-AMC (K_{M}^{app˜} 38 μM) was used as the substrate at a concentration equivalent to K_{M}^{app}. The rate of conversion of substrate to product was derived from the slope of the increase in fluorescence monitored continuously over time.

Cathepsin Peptidase Activity Assays

Bovine cathepsin S, human cathepsin L, human cathepsin H and human cathepsin B were obtained from CN Biosciences. Recombinant human cathepsin S, human cathepsin K and human cathepsin X were obtained from Dr. Boris Turk (Josef Stefan Institute, Ljubljana, Slovenia). Unless otherwise stated, all peptidase activity assays were carried out in 10 mM bis-tris-propane (BTP), pH 6.5 containing 1 mM EDTA, 5 mM 2-mercaptoethanol and 1 mM CaCl₂. Human cathepsin H activity assays were carried out in 10 mM BTP pH 6.5, 142 mM NaCl₂, 1 mM CaCl₂, 1 mM EDTA, 1 mM DTT, 0.035 mM Zwittergent 3-16. Human cathepsin K assays were carried out in 100 mM sodium acetate; pH 5.5 containing 20 mM L-cysteine and 1 mM EDTA (Bossard, M. J., et. al., J. Biol. Chem., 21, 12517-12524, 1996). Human cathepsin X assays were carried out in 100 mM sodium acetate; pH 5.5 containing 20 mM L-cysteine; 0.05% (w/v) Brij 35 and 1 mM EDTA (Santamaria, I., et. al., J. Biol. Chem., 273, 16816-16823, 1998; Klemencic, J, et al., Eur. J. Biochem., 267, 5404-5412, 2000). The final enzyme concentrations used in the assays were 0.5 nM bovine cathepsin S, 1 nM cathepsin L, 0.1 nM cathepsin B, 0.25 nM Cathepsin K; 1 nM cathepsin X and 10 nM cathepsin H. For the inhibition assays, the substrates used for cathepsin S, cathepsin L, cathepsin B, cathepsin K and cathepsin H were boc-Val-Leu-Lys-AMC (K_{M}^{app˜} 30 μM), Ac-Phe-Arg-AMC (K_{M}^{app˜} 20 μM, Z-Phe-Arg-AMC (K_{M}^{app˜} 40 μM), Z-Leu-Arg-AMC (K_{M}^{app˜} 2 μM); Bz-Phe-Val-Arg-AMC (K_{M}^{app˜} 150 μM) respectively. In each case the substrate concentration used in each assay was equivalent to the K_{M}^{app}. The rate of conversion of substrate to product was derived from the slope of the increase in fluorescence monitored continuously over time.

Trypsin Peptidase Activity Assays

Human pancreatic trypsin (iodination grade; CN Biosciences) activity assays were carried out in 10 mM HEPES, pH 8.0 containing 5 mM CaCl₂ using 0.1 nM trypsin. For the inhibition assays, Z-Gly-Gly-Arg-AMC (K_{M}^{app·} 84 μM) was used as the substrate at a concentration equivalent to K_{M}^{app}. The rate of conversion of substrate to product was derived from the slope of the increase in fluorescence monitored continuously over time.

Clostripain Peptidase Activity Assays

Clostripain (Sigma) activity assays were carried out in 10 mM BTP, pH 6.5 containing 1 mM EDTA, 5 mM 2-mercaptoethanol and 1 mM CaCl₂ using 0.3 nM enzyme. For the inhibition assays, Z-Gly-Gly-Arg-AMC (K_{M}^{app˜} 100 μM) was used as the substrate at a concentration equivalent to K_{M}^{app}. The rate of conversion of substrate to product was derived from the slope of the increase in fluorescence monitored continuously over time.

Calpain Peptidase Activity Assays

Calpain (human erythrocyte μ-calpain and porcine kidney m-calpain; CN Biosciences) activity assays were carried out in 10 mM HEPES, pH 7.5 containing 2 mM 2-mercaptoethanol and CaCl₂ using 25 nM of either enzyme (Sasaki, et. al., J. Biol. Chem., 259, 12489-12494, 1984). For μ-calpain inhibition assays, the buffer contained 100 μM CaCl₂ and Abz-Ala-Asn-Leu-Gly-Arg-Pro-Ala-Leu-Tyr(NO₂)-Asp-NH₂ (K_{M}^{app˜} 20 μM; Incenta Limited) was used as the substrate. For m-calpain inhibition assays, the assay buffer contained 200 μM CaCl₂ and Abz-Lys-Leu-Cys(Bzl)-Phe-Ser-Lys-Gln-Tyr(NO₂)-Asp-NH₂ (K_{M}^{app˜} 22 μM; Incenta Limited) was used as the substrate. In both cases the substrate concentration employed in the assays was equivalent to the K_{M}^{app}. The rate of conversion of substrate to product was derived from the slope of the increase in fluorescence monitored continuously over time.

Extracellular S. aureus V8 Cysteine Peptidase (Staphylopain) Peptidase Activity Assays

S. aureus V8 was obtained from Prof S. Aridson, Karolinska Institute, Stockholm, Sweden. Extracellular S. aureus V8 cysteine peptidase (staphylopain) activity assays were carried out using partially purified S. aureus V8 culture supernatant (obtained from Dr. Peter Lambert, Aston University, Birmingham, U.K.). Activity assays were carried out in 10 mM BTP, pH 6.5 containing 1 mM EDTA, 5 mM 2-mercaptoethanol and 1 mM CaCl₂ using two-times diluted partially purified extract. For the inhibition assays, Abz-Ile-Ala-Ala-Pro-Tyr(NO₂)-Glu-NH₂ (K_{M}^{app˜} 117 μM; Incenta Limited) was used as the substrate at a concentration equivalent to K_{M}^{app}. The rate of conversion of substrate to product was derived from the slope of the increase in fluorescence monitored continuously over time.

Foot-and-Mouth Disease Leader Peptidase (FMDV-LP) Activity Assays

Recombinant wild-type FMDV-LP was obtained from Dr. Tim Skern (Institut für Medizinische Biochemie, Abteilung für Biochemie, Universtät Wien, Wien, Austria). Activity assays were carried out in 50 mM tris.acetate, pH 8.4 containing 1 mM EDTA, 10 mM L-cysteine and 0.25% (w/v) CHAPS using 10 nM enzyme. For the inhibition assays, Abz-Arg-Lys-Leu-Lys-Gly-Ala-Gly-Ser-Tyr(NO₂)-Glu-NH₂ (K_{M}^{app˜} 51 μM, Incenta Limited) was used as the substrate at a concentration equivalent to K_{M}^{app}. The rate of conversion of substrate to product was derived from the slope of the increase in fluorescence monitored continuously over time.

Caspase Peptidase Activity Assays

Caspases 1-10 were obtained from CN Biosciences or BioVision Inc. (Mountain View, Calif., USA) and all assays were carried out in 50 mM HEPES; pH 7.2, 10% (v/v) glycerol, 0.1% (w/v) CHAPS, 142 mM NaCl, 1 mM EDTA, 5 mM dithiothreitol (DTT) using 0.1-1 Upper assay. For caspase 1, Ac-Leu-Glu-His-Asp-AMC was used as the substrate; for caspase 2, Z-Val-Asp-Val-Ala-Asp-AFC was used as the substrate; for caspase 3, Ac-Asp-Glu-Val-Asp-AMC was used as the substrate; for caspase 4, Suc-Tyr-Val-Ala-Asp-AMC was used as the substrate; for caspase 5, Ac-Leu-Glu-His-Asp-AMC was used as the substrate; for caspase 6, Ac-Val-Glu-Ile-Asp-AMC was used as the substrate; for caspase 7, Ac-Asp-Glu-Val-Asp-AMC was used as the substrate; for caspase 8, Ac-Ile-Glu-Thr-Asp-AMC was used as the substrate; for caspase 9, Ac-Leu-Glu-His-Asp-AMC was used as the substrate; for caspase 10, Ac-Ile-Glu-Tbr-Asp-AMC was used as the substrate (Nicholson, D. W. and Thornberry, N. A., TIBS, 22, 299-306, 1997; Stennicke, H. R. and Salvesen, G. S., J. Biol. Chem., 272(41), 25719-25723, 1997; Talanian, R. V., et. al., J. Biol. Chem., 272(15), 9677-9682, 1997; Wolf, B. B. and Green, D. R., J. Biol. Chem., 274(29), 20049-20052, 1999). The rate of conversion of substrate to product was derived from the slope of the increase in fluorescence monitored continuously over time.

Measurement of the Apparent Macroscopic Binding (Michaelis) Constants (K_{M}^{app}) for Substrates

The apparent macroscopic binding constant (K_{M}^{app}) for each substrate was calculated, from the dependence of enzyme activity as a function of substrate concentration. The observed rates were plotted on the ordinate against the related substrate concentration on the abscissa and the data fitted by direct regression analysis (Prism v 3.02; GraphPad, San Diego, USA) using Equation 1 (Cornish-Bowden, A. Fundamentals of enzyme kinetics Portland Press; 1995, 93-128.).

$${v}_{i} = \frac{{V}_{max}^{app} \cdot [ {S}_{o} ]}{[ {S}_{o} ] + {K}_{M}^{app}} \qquad ( 1 )$$

In Equation 1 ‘vᵢ’ is the observed initial rate, ‘Vₘₐₓ^{app}’ is the observed maximum activity at saturating substrate concentration, ‘K_{M}^{app}’ is the apparent macroscopic binding (Michaelis) constant for the substrate, ‘[S₀]’ is the initial substrate concentration.

Measurement of the Inhibition Constants

The apparent inhibition constant (Kᵢ) for each compound was determined on the basis that inhibition was reversible and occurred by a pure-competitive mechanism. The Kᵢ values were calculated, from the dependence of enzyme activity as a function of inhibitor concentration, by direct regression analysis (Prism v 3.02) using Equation 2 (Cornish-Bowden, A., 1995.).

$${v}_{i} = \frac{{V}_{max}^{app} \cdot [ S ]}{[ S ] + \{ {K}_{M}^{app} \cdot ( [ I ] / {K}_{i} ) \}} \qquad ( 2 )$$

In Equation 2 ‘vᵢ’ is the observed residual activity, ‘Vₘₐₓ^{app}’ is the observed maximum activity (i.e. in the absence of inhibitor), ‘K_{M}^{app}’ is the apparent macroscopic binding (Michaelis) constant for the substrate, ‘[S]’ is the initial substrate concentration, ‘K₁’ is the apparent dissociation constant and ‘[I]’ is the inhibitor concentration.

In situations where the apparent dissociation constant (Kᵢ^{app}) approached the enzyme concentrations, the Kᵢ^{app} values were calculated using a quadratic solution in the form described by Equation 3 (Morrison, J. F. Trends Biochem. Sci., 7, 102-105, 1982; Morrison, J. F. Biochim. Biophys. Acta, 185, 269-286, 1969; Stone, S. R. and Hofsteenge, J. Biochemistry, 25, 4622-4628, 1986).

$${v}_{i} = \frac{\begin{matrix}F \{ {E}_{o} - {I}_{o} - {K}_{i}^{app} + \\ \sqrt{( {E}_{o} - {I}_{o} - {K}_{i}^{app} ) + 4 \cdot {K}_{i}^{app} \cdot {E}_{o}} \}\end{matrix}}{2} \qquad ( 3 )$$

Kᵢ^{app}=Kᵢ(1+[Sₒ]/K_{M}^{app}) (4)

In Equation 3 ‘v₁’ is the observed residual activity, ‘F’ is the difference between the maximum activity (i.e. in the absence of inhibitor) and minimum enzyme activity, ‘Eₒ’ is the total enzyme concentration, ‘Kᵢ^{app}’ is the apparent dissociation constant and ‘Iₒ’ is the inhibitor concentration. Curves were fitted by non-linear regression analysis (Prism) using a fixed value for the enzyme concentration. Equation 4 was used to account for the substrate kinetics, where ‘K₁’ is the inhibition constant, ‘[Sₒ]’ is the initial substrate concentration and ‘K_{M}^{app}’ is the apparent macroscopic binding (Michaelis) constant for the substrate (Morrison, 1982).

The Second-Order Rate of Reaction of Inhibitor with Enzyme

Where applicable, the concentration dependence of the observed rate of reaction (k_{obs}) of each compound with enzyme was analysed by determining the rate of enzyme inactivation under pseudo-first order conditions in the presence of substrate (Morrison, J. F., TIBS, 102-105, 1982; Tian, W. X. and Tsou, C. L., Biochemistry, 21, 1028-1032, 1982; Morrison, J. F. and Walsh, C. T., from Meister (Ed.), Advances in Enzymol., 61, 201-301, 1988; Tsou, C. L., from Meister (Ed.), Advances in Enzymol., 61, 381-436, 1988;). Assays were carried out by addition of various concentrations of inhibitor to assay buffer containing substrate. Assays were initiated by the addition of enzyme to the reaction mixture and the change in fluorescence monitored over time. During the course of the assay less than 10% of the substrate was consumed.

$$F = {v}_{s} t + \frac{( {v}_{o} - {v}_{s} ) ⌊ 1 - {ⅇ}^{( {k}_{obs} \cdot t )} ⌋}{{k}_{obs}} + D \qquad ( 5 )$$

The activity fluorescence progress curves were fitted by non-linear regression analysis (Prism) using Eq. 5 (Morrison, 1969; Morrison, 1982); where ‘F’ is the fluorescence response, ‘t’ is time, ‘vₒ’ is the initial velocity, ‘vₛ’ is the equilibrium steady-state velocity, ‘k_{obs}’ is the observed pseudo first-order rate constant and ‘D’ is the intercept at time zero (i.e. the ordinate displacement of the curve). The second order rate constant was obtained from the slope of the line of a plot of k_{obs} versus the inhibitor concentration (i.e. k_{obs}/[I]). To correct for substrate kinetics, Eq. 6 was used, where ‘[Sₒ]’ is the iniitial substrate concentration and ‘K_{M}^{app}’ is the apparent macroscopic binding (Michaelis) constant for the substrate.

$${k}_{inact} = \frac{{k}_{obs} ( 1 + [ {S}_{o} ] / {K}_{M}^{app} )}{[ I ]} \qquad ( 6 )$$

Compounds of the invention were tested by the above described assays and observed to exhibit cathepsin K inhibitory activity or inhibitory activity against an alternative CA C1 cysteine protease with an in vitro Ki inhibitory constant of less than or equal to 100 μM. Exemplary inhibition data for examples of the invention are given in Table 5.

TABLE 5
Exemplary inhibition data (Ki expressed as μM).

| Example | Human | | Bovine | Human | |
|---|---|---|---|---|---|
| N° | Cathepsin K | Cruzipain | Cathepsin S | Cathepsin L | CPB |
| 2 | <0.01 | >0.3 | >1 | >3 | >0.2 |
| 296 | >50 | >1 | >5 | <0.2 | >5 |
| 250 | >5 | >5 | <0.1 | >1 | >5 |
| 346 | >8 | <0.2 | >10 | >3 | >5 |

Human Osteoclast Resorption Assay

Bone resorption was studied using a model where human osteoclast precursor cells were cultured on bovine bone slices for 9 days and allowed to differentiate into bone-resorbing osteoclasts. The formed mature osteoclasts were then allowed to resorb bone. The assay was performed by Pharmatest Services Ltd, Itainen Pitkakatu 4C, Turku, Finland. After the culture period, bone collagen degradation products were quantified from the culture medium as an index of bone resorption. Inhibitor compounds were added into the cell cultures after the differentiation period and their effects on the resorbing activity of mature osteoclasts were determined. The studies included a baseline group without added compounds and a positive control group where a potent cathepsin K inhibitor E-64 was added.

Human peripheral blood monocytes were suspended to culture medium and allowed to attach to bovine bone slices. The bone slices were transferred into 96-well tissue culture plates containing culture medium with appropriate amounts of important growth factors favouring osteoclast differentiation, including M-CSF, RANK-ligand and TGF-β. The cells were incubated in a CO₂ incubator in humidified atmosphere of 95% air and 5% carbon dioxide at 37° C. At day 7 when osteoclast differentiation was complete, the culture medium was replaced with culture medium containing conditions favouring osteoclast activity. The cell culture was continued for an additional 2 days, during which the formed mature osteoclasts were allowed to resorb bone in the presence of vehicle, control inhibitor (E64) or test compounds. At the end of the culture, bone collagen degradation products released into the culture medium were determined using a commercially available ELISA method (CrossLaps® for culture, Nordic Bioscience, Herlev, Denmark) as an index of bone resorption (see Bagger, Y. Z. et al, J. Bone. Miner. Res. 14 (suppl. 1), S370).

In this assay, selected EXAMPLES of the invention exhibited more than 70% inhibition of bone resorption at a concentration of 100 nM.

1. A compound of general formula (I) 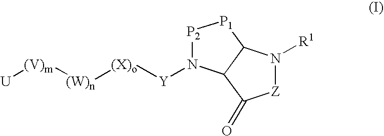
wherein:
Z=CR³R⁴, where R³ and R⁴ are independently chosen from C₀₋₇-alkyl, C₃₋₆-cycloalkyl, and Ar—C₀₋₇-alkyl,
P₁=CR⁵R⁶, where R⁵ and R⁶ are independently chosen from C₀₋₇-alkyl, C₃₋₆-cycloalkyl, Ar—C₀₋₇-alkyl, O—C₀₋₇-alkyl, O—C₃₋₆-cycloalkyl, O—Ar—C₀₋₇-alkyl, S—C₀₋₇-alkyl, S—C₃₋₆-cycloalkyl, S—Ar—C₀₋₇-alkyl, NH—C₀₋₇-alkyl, NH—C₃₋₆-cycloalkyl, NH—Ar—C₀₋₇-alkyl, N(C₀₋₇-alkyl)₂, N(C₃₋₆-cycloalkyl)₂, and N(Ar—C₀₋₇-alkyl)₂;
P₂=O, CR⁷R⁸ or NR⁹, where R⁷ and R⁸ are independently chosen from C₀₋₇-alkyl, C₃₋₆-cycloalkyl, and Ar—C₀₋₇-alkyl, and R⁹ is chosen from C₀₋₇-alkyl, C₃₋₆-cycloalkyl and Ar—C₀₋₇-alkyl;
Y=CR¹⁰R¹¹—C(O), CR¹⁰R¹¹—C(S), CR¹⁰R¹¹—S(O), or CR¹⁰R¹¹—SO₂ where R¹⁰ and R¹¹ are independently chosen from C₀₋₇-alkyl, C₃₋₆-cycloalkyl and Ar—C₀₋₇-alkyl, or Y is 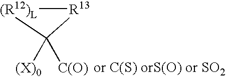
where L is an integer from one to four and R¹² and R¹³ are each CR¹⁴R¹⁵ where R¹⁴ and R¹⁵ are each independently chosen from C₀₋₇-alkyl, C₃₋₆-cycloalkyl, Ar—C₀₋₇-alkyl, and halogen; and for each R¹² and R¹³ either R¹⁴ or R¹⁵ (but not both R¹⁴ and R¹⁵) may additionally be chosen from O—C₀₋₇-alkyl, O—C₃₋₆-cycloalkyl, O—Ar—C₀₋₇-alkyl, S—C₀₋₇-alkyl, S—C₃₋₆-cycloalkyl, S—Ar—C₀₋₇-alkyl, NH—C₀₋₇-alkyl, NH—C₃₋₆-cycloalkyl, NH—Ar—C₀₋₇-alkyl, N—(C₀₋₇-alkyl)₂, N—(C₃₋₆-cycloalkyl)₂, and N—(Ar—C₀₋₇-alkyl)₂;
(X)ₒ=CR¹⁶R¹⁷, where R¹⁶ and R¹⁷ are each independently chosen from C₀₋₇-alkyl, C₃₋₆-cycloalkyl, and Ar—C₀₋₇-alkyl, and o is an integer from zero to three;
(W)ₙ=O, S, C(O), S(O), S(O)₂, or NR¹⁸, where R¹⁸ is chosen from C₀₋₇-alkyl, C₃₋₆-cycloalkyl, and Ar—C₀₋₇-alkyl, and n is zero or one;
(V)ₘ═C(O), C(S), S(O), S(O)₂, S(O)₂NH, OC(O), NHC(O), NHS(O), NHS(O)₂, OC(O)NH, C(O)NH, CR¹⁹R²⁰, C═N—C(O)—OR¹⁹, or C═N—C(O)—NHR¹⁹, where R¹⁹ and R²⁰ are each independently chosen from C₀₋₇-alkyl, C₃₋₆-cycloalkyl, and Ar—C₀₋₇-alkyl, and m is an integer from zero to three, provided that when m is greater than one, (V)ₘ contains a maximum of one carbonyl or one sulphonyl group;
U=a stable 5- to 7-membered monocyclic or a stable 8- to 11-membered bicyclic ring which is either saturated or unsaturated and which includes zero to four heteroatoms, and wherein U is chosen from: 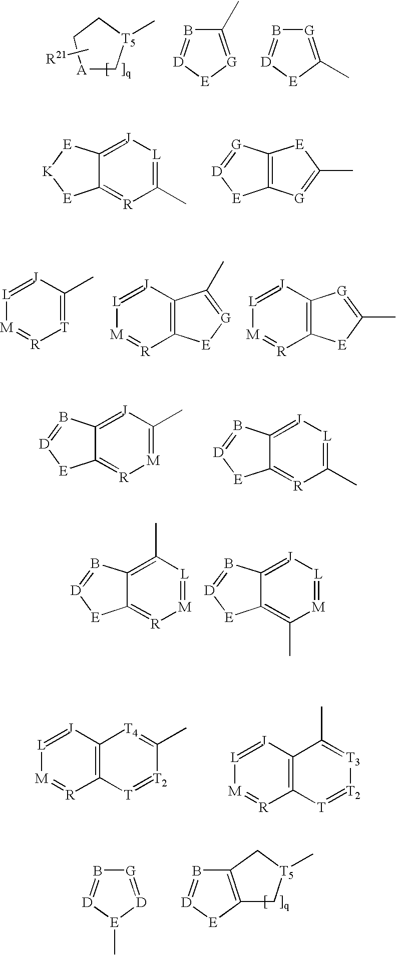 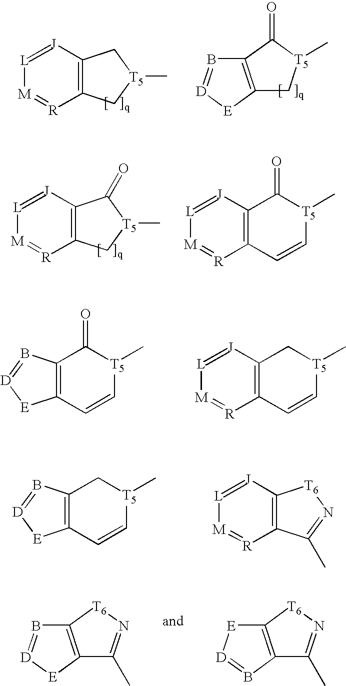
wherein R²¹ is:
C₀₋₇-alkyl, C₃₋₆-Cycloalkyl, Ar—C₀₋₇-alkyl, O—C₀₋₇-alkyl, O—C₃₋₆-cycloalkyl, O—Ar—C₀₋₇-alkyl, S—C₀₋₇-alkyl, S—C₃₋₆-cycloalkyl, S—Ar—C₀₋₇-alkyl, SO₂—C₀₋₇-alkyl, SO₂—C₃₋₆-cycloalkyl, SO₂—Ar—C₀₋₇-alkyl, NH—C₀₋₇-alkyl, NH—C₃₋₆-cycloalkyl, NH—Ar—C₀₋₇-alkyl, N(C₀₋₇-alkyl)₂, N(C₃₋₆-cycloalkyl)₂ or N(Ar—C₀₋₇-alkyl)₂; or, when part of a CHR²¹ or CR²¹ group, R²¹ may be halogen;
A is chosen from:
CH₂, CHR²¹, O, S, SO₂, NR²² or N-oxide (N→O), where R²¹ is as defined above; and R²² is chosen from C₀₋₇-alkyl, C₃₋₆-cycloalkyl, and Ar—C₀₋₇-alkyl;
B, D, and G are each independently chosen from:
CR²¹, where R²¹ is as defined above, N or N-oxide (N→O);
E is chosen from:
CH₂, CHR²¹, O, S, SO₂, NR²², and N-oxide (N→O), where R²¹ and R²² are defined as above;
K is chosen from:
CH₂, or CHR²², where R²² is defined as above;
J, L, M, R, T, T₂, T₃ and T₄ are independently chosen from:
CR²′ where R²¹ is as defined above, N and N-oxide (N→O);
T₅ is chosen from:
CH and N;
T₆ is chosen from:
NR²², SO₂, OC(O), C(O), and NR²²C(O);
q is a number from one to three, thereby defining a 5-, 6- or 7-membered ring;
R¹═R²C(O), R²OC(O), R²NQC(O), R²SO₂, where R² is chosen from C₁₋₇-alkyl, C₃₋₆-cycloalkyl, and Ar—C₀₋₇-alkyl, and Q is C₀₋₇-alkyl;
provided that when Y is other than CR¹⁰R¹¹—C(O) or when U is: 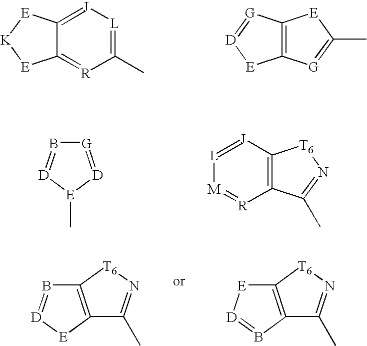
R¹ may also be C₀₋₇-alkyl, C₃₋₆-cycloalkyl or Ar—C₀₋₇-alkyl;
wherein C₀ is hydrogen:
or a salt, hydrate, solvate, complexe or prodrug thereof.

2. A compound as claimed in claim 1 wherein independently or in any combination:
Z is CH₂;
P¹ is CH₂; and
P² is CH₂, O or NH.

3. A compound as claimed in claim 1 wherein R² is Ar—C₀₋₂-alkyl.

4. A compound as claimed in claim 3, wherein R² is: 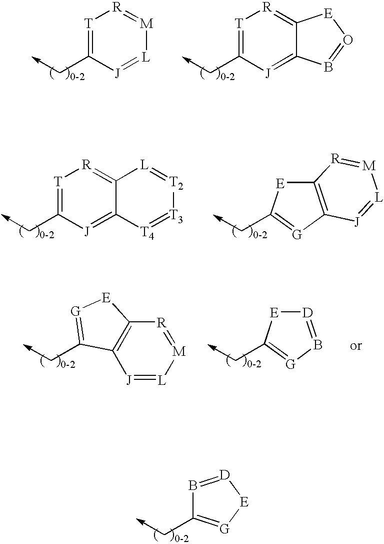
where B, D, G, E, J, L, M, R, T, T₂, T₃, and T₄, are as previously defined.

5. A compound as claimed in claim 1, wherein R² is Ar—C₀₋₁-alkyl.

6. A compound as claimed in claim 5, wherein R² is: 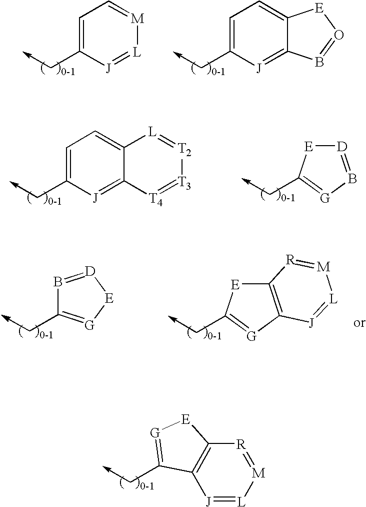
where B, D, G, E, J, L, M, T₂, T₃, and T₄, B, D, G and E are as previously defined.

7. A compound as claimed in claim 1 wherein R² comprises a monocyclic Ar—C₀₋₁-alkyl.

8. A compound as claimed in claim 7, wherein R² forms part of an R¹ group selected from: 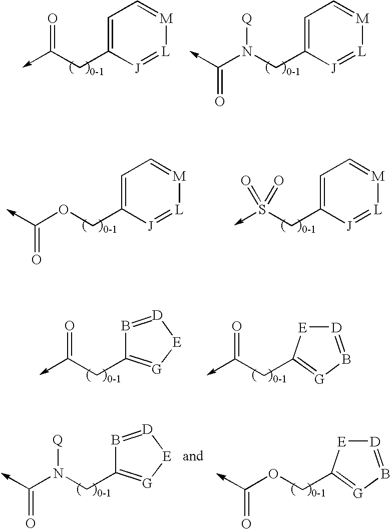
wherein:
B, D, E, G, J, L, and M, are as defined above;
R²¹ is chosen from hydrogen, methyl, methoxy, ethyl, isopropyl, trifluoromethyl, trifluoromethoxy, F, Cl, and SO₂Me; and
Q is chosen from hydrogen and methyl.

9. A compound as claimed in claim 1 or claim 2 wherein R² is C₃₋₇-alkyl which optionally includes an —O— or —NH— as part of the chain and which is either unsubstituted or is substituted with one or more NH₂, NHMe, NHC(O)CH₃, NMeC(O)CH₃, OH, or OMe groups.

10. A compound as claimed in claim 9 wherein when R² is a C₃₋₆-alkyl group which is branched at the α-position or which includes an NH₂, NHMe, NHC(O)CH₃, NMeC(O)CH₃, OH, or OMe substituent at the α-position.

11. A compound as claimed in claim 1 or claim 2 wherein R² is cyclopropyl, cyclobutyl, cyclopentyl, cyclohexyl, pyrrolidine, piperidine, morpholine, tetrahydrofuran, cyclopentene, cyclopentadiene, cyclohexadiene or piperazine, wherein nitrogen-containing rings may be N-substituted with C₁₋₄ alkyl, phenyl, or benzyl.

12. A compound as claimed in claim 1 or claim 2, wherein R² is a C₃₋₄-cycloalkyl group, wherein the ring system is either connected directly to the remainder of the R¹ moiety or there is one intervening methylene group.

13. A compound as claimed in claim 1 or claim 2 wherein R¹ is: benzoyl; pyridine-2-carbonyl; 1-oxy-pyridine-2-carbonyl; pyridine-3-carbonyl; 1-oxy-pyridine-3-carbonyl; pyridine-4-carbonyl; 1-oxy-pyridine-4-carbonyl; phenyl sulphonyl; pyridine-2-sulphonyl; 1-oxy-pyridine-2-sulphonyl; pyridine-3-sulphonyl; 1-oxy-pyridine-3-sulphonyl; pyridine-4-sulphonyl; 1-oxy-pyridine-4-sulphonyl; phenylacetyl; phenylcarbamoyl; isobutylcarbamoyl; phenyloxycarbonyl; isobutyloxycarbonyl; pyrrolidine-N-carbonyl; piperidine-N-carbonyl; morpholin-N-carbonyl; piperazine-N-carbonyl; 4-methyl-piperazine-N-carbonyl; (4-methyl-piperazin-1-yl)-acetoyl; piperazin-1-yl-acetoyl; furan-2-carbonyl; 5-chlorofuran-2-carbonyl; thiophene-2-carbonyl; 5-chlorothiophene-2-carbonyl; furan-3-carbonyl; thiophene-3-carbonyl; cyclopentoyl; cyclohexoyl; cyclopent-3-enoyl; cyclopentylmethylcarbonyl; cyclohexylmethylcarbonyl; pyrrolidine-2-carbonyl; N-acetyl-pyrrolidine-2-carbonyl; piperidine-2-carbonyl; N-acetyl-piperidine-2-carbonyl; tetrahydrofuran-2-carbonyl; 1-aminocyclobutanoyl; 1-aminocyclopentanoyl; 1-aminocyclohexanoyl; N-acetyl-1-aminocyclobutanoyl; N-acetyl-1-aminocyclopentanoyl; N-acetyl-1-aminocyclohexanoyl; 1-hydroxycyclobutanoyl; 1-hydroxycyclopentanoyl; 1-hydroxycyclohexanoyl; 1-methoxycyclobutanoyl; 1-methoxycyclopentanoyl; 1-methoxycyclohexanoyl; aminocyclopentylacetoyl; aminocyclohexylacetoyl; N-acetylaminocyclopentylacetoyl; N-acetylaminocyclohexylacetoyl; 2-acetylaminopropionoyl; 2-acetylaminoethanoyl; 2-acetyl-N-methylaminoethanoyl; N,N-dimethylaminoacetoyl; 2-aminobutanoyl; N-acetyl-2-aminobutanoyl; 2-amino-3-methylbutanoyl; N-acetyl-2-amino-3-methylbutanoyl; 2-amino-3,3-dimethylbutanoyl; N-acetyl-2-amino-3,3-dimethylbutanoyl; 2-amino-3-methylpentanoyl; N-acetyl-2-amino-3-methylpentanoyl; pentanoyl; 3-methylpentanoyl; 4-methylpentanoyl; 2-amino-4-methylpentanoyl; N-acetyl-2-amino-4-methylpentanoyl; 2-amino-4,4-dimethylpentanoyl; N-acetyl-2-amino-4,4-dimethylpentanoyl; 2-aminopentanoyl; N-acetyl-2-aminopentanoyl; 2-amino-5-methylhexanoyl; N-acetyl-2-amino-5-methylhexanoyl; 2-hydroxy-3-methylbutanoyl; 2-methoxy-3-methylbutanoyl; 2-hydroxy-3,3-dimethylbutanoyl; 2-methoxy-3,3-dimethylbutanoyl; 2-hydroxy-3-methylpentanoyl; 2-methoxy-3-10 methylpentanoyl; 2-hydroxy-4-methylpentanoyl; 2-methoxy-4-methylpentanoyl; 2-hydroxy-4,4-dimethylpentanoyl; 2-methoxy-4,4-dimethylpentanoyl; 2-hydroxypentanoyl; 2-methoxypentanoyl; 2-hydroxy-5-methylhexanoyl; or 2-methoxy-5-methylhexanoyl;

14. A compound as claimed in claim 1, wherein, in the group (X)ₒ, each of R¹⁶ and R¹⁷ is independently selected from the group consisting of C₀₋₇-alkyl and Ar—C₀₋₇-alkyl.

15. A compound as claimed in claim 14, wherein, in the group (X)ₒ, R¹⁶ is hydrogen; R¹⁷ is chosen from hydrogen, C₁₋₄-alkyl, and Ar—C₁₋₄-alkyl;
wherein the aryl group of Ar—C₁₋₄ alkyl may optionally be substituted with R²¹;
wherein C₁₋₄-alkyl may optionally be substituted with OH, NR²²R²², COOR²², or CONR²²; and
wherein each R²¹ and R²² is as previously defined.

16. A compound as claimed in claim 15 wherein, in the group (X)ₒ, R¹⁶ is hydrogen and R¹⁷ is chosen from hydrogen and C₁₋₄-alkyl.

17. A compound as claimed in claim 16 wherein, in the group (X)ₒ, R¹⁶ and R¹⁷ are hydrogen and o is zero or one.

18. A compound as claimed in claim 1 wherein, in the group (W)ₙ, W is chosen from O, S, SO₂, S(O), C(O) and NR¹⁸, where R¹⁸ is C₀₋₇-alkyl; and n is zero or one.

19. A compound as claimed in claim 18, wherein, in the group (W)ₙ, W is chosen from O, S, SO₂, C(O) and NH; and n is zero or one.

20. A compound as claimed in claim 19 wherein, in the group (W)ₙ, W is C(O) or NH; and n is zero or one.

21. A compound as claimed in claim 20 wherein in the group (W)ₙ, W is NH and n is zero or one.

22. A compound as claimed in claim 1 wherein, in the group (V)ₘ, V is chosen from C(O), OC(O), NHC(O), C(O)NH, CHR²⁰, C═N—C(O)—OR¹⁹ and C═N—C(O)—NHR¹⁹; R¹⁹ is chosen from C₀₋₇-alkyl, C₃₋₆-cycloalkyl, and Ar—C₀₋₇-alkyl; R²⁰ is C₀₋₄-alkyl; and m is zero or one.

23. A compound as claimed in claim 1, wherein the V and W substituent combination comprises:

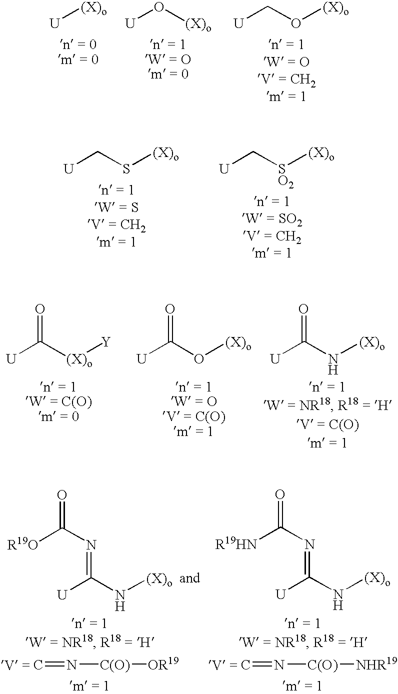

24. A compound as claimed in claim 1 wherein the V, W, and X substituent combination comprises:

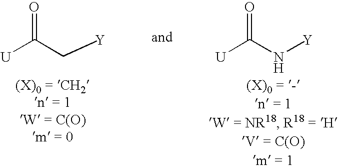

25. A compound as claimed in claim 1 wherein Y is CHR¹¹CO; where R¹¹ is selected from C₀₋₇-alkyl, Ar—C₀₋₇-alkyl, and C₃₋₆-cycloalkyl; or
wherein Y comprises the group: 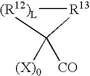
where R¹² and R¹³ are each CR¹⁴R¹⁵ and each R¹⁴ and R¹⁵ is, independently, selected from C₀₋₇-alkyl and Ar—C₀₋₇-alkyl; and L is an integer from one to four.

26. A compound as claimed in claim 25 wherein Y is selected from the group consisting of: 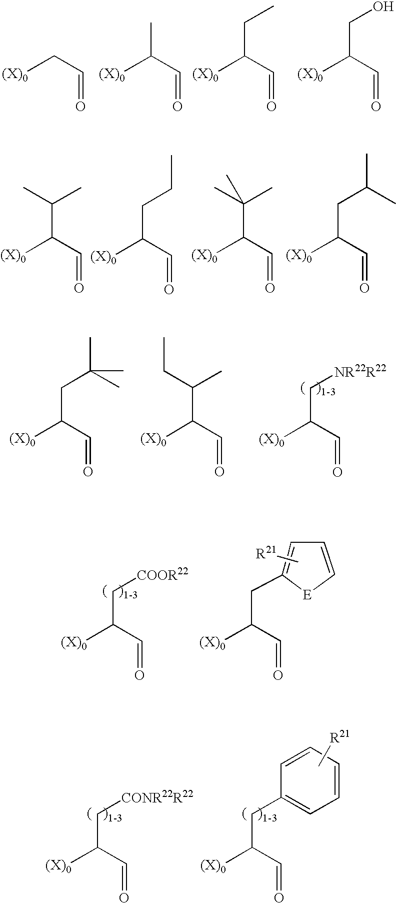 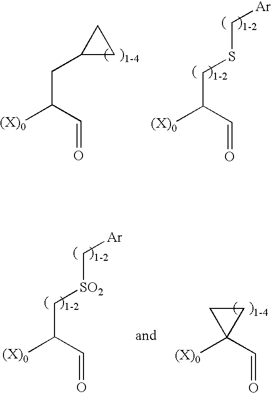
wherein E, R²¹, R²² and Ar are as defined previously.

27. A compound as claimed in claim 25, wherein, in the group Y, R¹¹ is C₁₋₄-alkyl, or cycloalkyl-1-carbonyl or Ar—C₁₋₄-alkyl;
wherein the C₁₋₄-alkyl may be substituted with cycloalkylmethyl or halogen; the aryl group may be substituted with R²¹; and
where R²¹ is as previously defined.

28. A compound as claimed in claim 27, wherein, in the group Y, R¹¹ is a straight or branched alkyl group, optionally substituted with one or more halogen substitutents.

29. A compound as claimed in claim 28, wherein in the group Y, R¹¹ is ArCH₂—, where the aromatic ring is an optionally substituted monocyclic heterocycle.

30. A compound as claimed in claim 25 wherein Y is selected from the group consisting from: 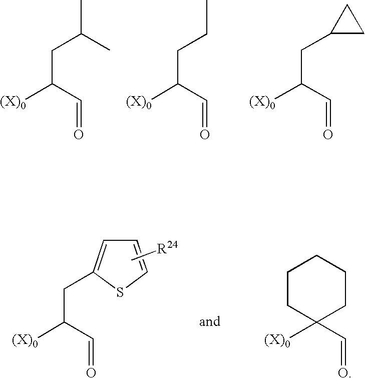
wherein R²⁴ is chosen from hydrogen, methyl, methoxy, ethyl, isopropyl, F, and Cl; and
wherein any of the alkyl groups may optionally be substituted with one or more F or Cl.

31. A compound as claimed in claim 1, U comprises: an optionally substituted 5- or 6-membered saturated or unsaturated monocyclic ring which optionally includes zero to four heteroatoms and an optionally substituted 8 to 10-membered saturated or unsaturated bicyclic ring which optionally includes zero to four heteroatoms, wherein the monocyclic or bicyclic ring is selected from the group consisting of 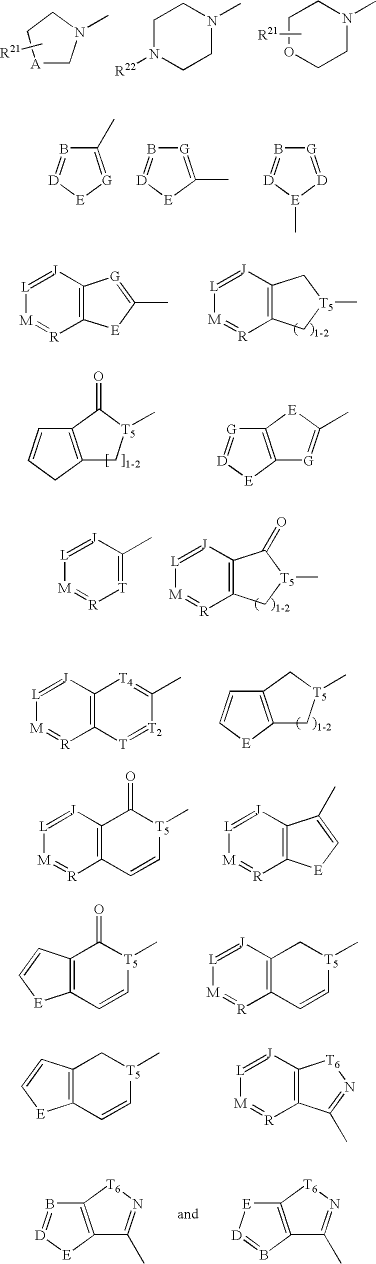
wherein R²¹, R²², A, B, D, E, G, J, L, M, R, T, T₂, T₄, T₅, and T₆ are as previously defined.

32. (canceled)

33. A compound as claimed in claim 31, wherein U comprises a bulky alkyl or aryl group at the para position of an aryl; a meta or para 5,6-biaryl, where Ar is as previously defined; a 6,6 or 6,5 or 5,5-fused aromatic ring, where Ar is as previously defined; or a 4-substituted piperazine; wherein U is selected from the group consisting of: 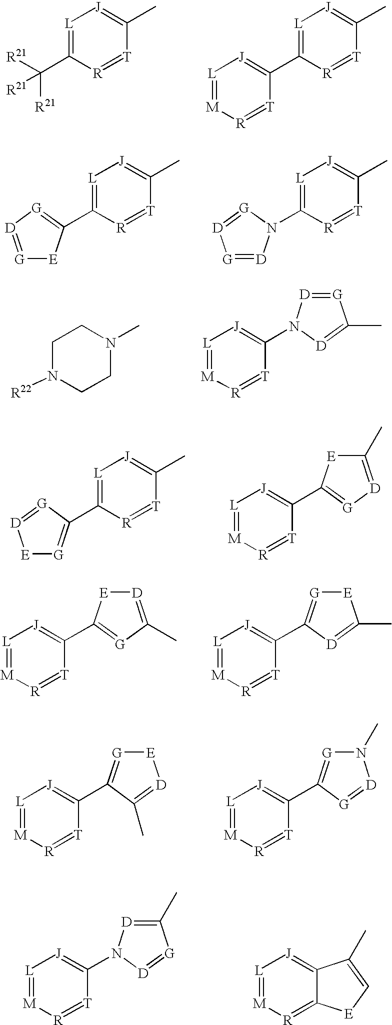 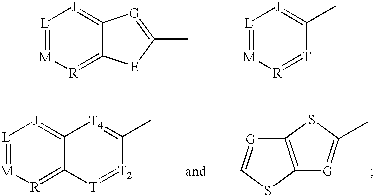
wherein R²¹, R²², D, E, G, J, L, M, R, T, T₂, and T₄ are as defined previously.

34. (canceled)

35. A compound as claimed in claim 33, wherein U comprises a 6-membered aromatic ring containing a bulky alkyl or aryl group at the para position; a meta or para-biaryl; a 6,6 or 6,5 or 5,5-fused aromatic ring; or a 4-substituted piperazine wherein U is selected from the group consisting of: 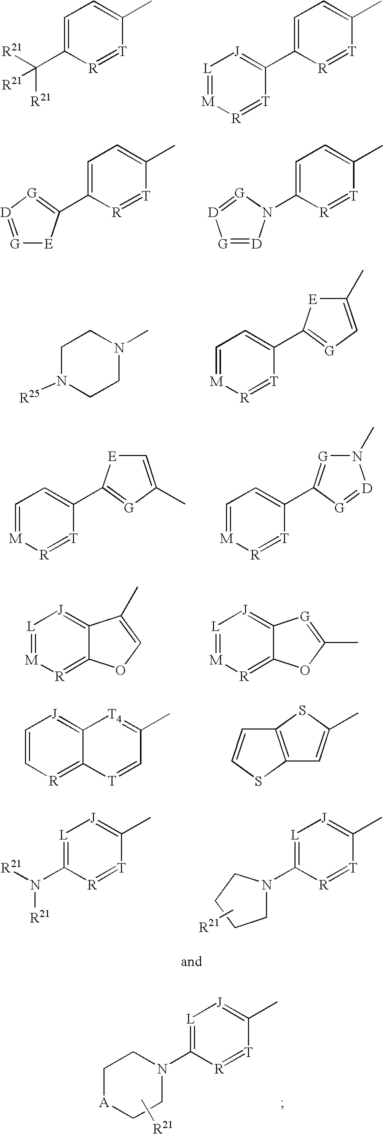
wherein R²⁵ is chosen from hydrogen, C₁₋₂-alkyl or Ar—C₀₋₂-alkyl; and
wherein R²¹, D, E, G, J, L, M, R, T, and T₄ are as previously defined.

36. (canceled)

37. A compound as claimed in claim 35, wherein U comprises: 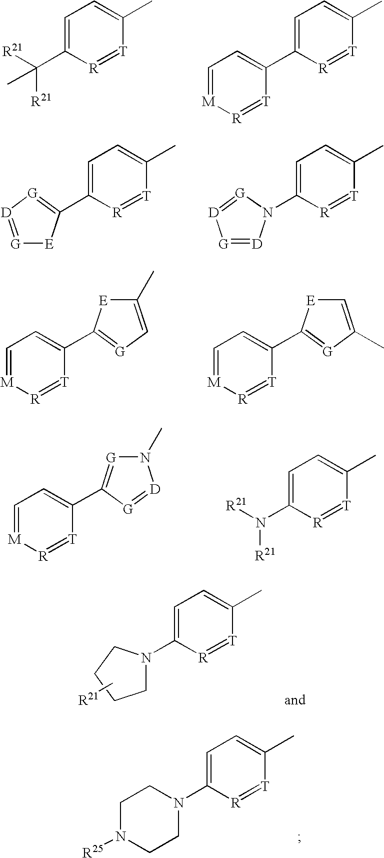
wherein R²¹, R²⁵, D, E, G, M, R, and T are as previously defined.

38. A compound as claimed in claim 1 which is an inhibitor of cathepsin S and wherein Y is selected from the group consisting of: 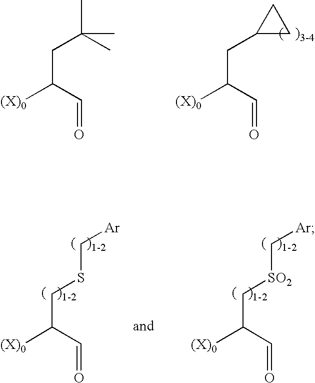
wherein (X)ₒ is as previously defined.

39. A compound as claimed in claim 38, wherein U comprises an optionally substituted 5-membered unsaturated heterocycle, a 6,5- or 5,5- or 5,6-fused aromatic ring, where Ar is as previously defined; or a morpholine; wherein U is selected from the group consisting of: 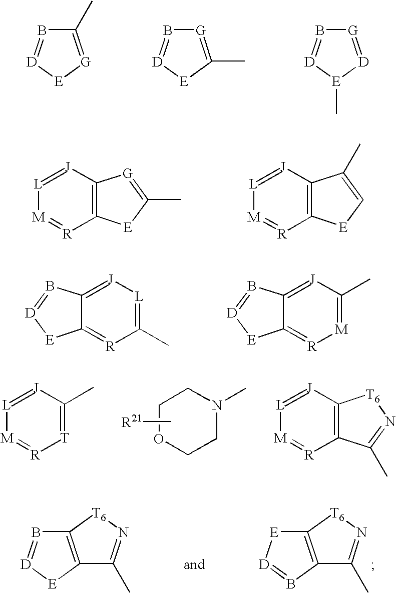
wherein R¹¹, B, D, E, G, J, L, M, R, and T₆ are as previously defined.

40. (canceled)

41. A compound as claimed in claim 40, wherein U comprises an optionally substituted 5-membered unsaturated heterocycle or a 6,5- or 5,5-fused aromatic ring wherein U is selected from the group consisting of: 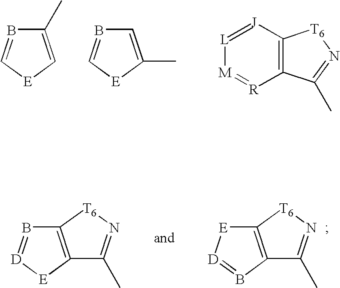
wherein B, D, E, J, L, M, R, and T₆ are as previously defined.

42. (canceled)

43. A compound as claimed in claim 1 wherein Y is: 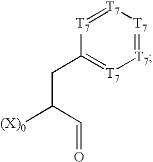
wherein T₇ is chosen from CH, and CR²¹ where R²¹ is as previously defined.

44. A compound as claimed in claim 43, wherein, within the T₇ substituent, each R²¹ independently is chosen from Me, F, Cl, OH, and OMe.

45. A compound as claimed in claim 43, wherein U comprises an optionally substituted 5-membered unsaturated heterocycle, a 6,6- or 6,5- or 5,6-fused aromatic ring, or a meta-substituted Ar; wherein U is selected from the group consisting of: 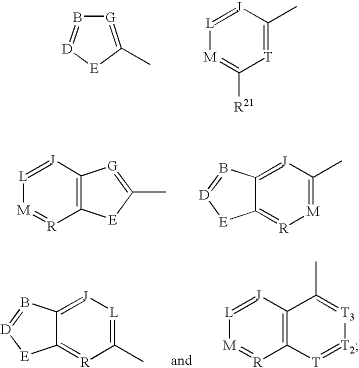
wherein R²¹, B, D, E, G, J, L, M, R, T, T₂, and T₃ are as previously defined.

46. (canceled)

47. A compound as claimed in claim 43, wherein U comprises a substituted 5-membered unsaturated heterocycle, a 6,6-fused aromatic ring, where Ar is as previously defined, or a meta-substituted Ar; wherein U is selected from the group consisting of: 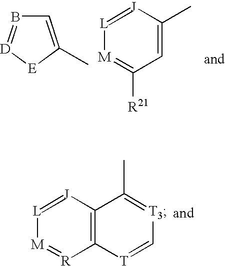
wherein
E is oxygen or N-ethyl,
D is nitrogen or CCH₃,
B is nitrogen or CCH₃,
R²¹ is halogen, OMe, CF₃, OCF₃, or CH₂NH₂; and
wherein J, L, M, R, T, and T₃ are as previously defined.

48. (canceled)

49. A compound as claimed in claim 1 which is a cis-bicyclic isomer.

50. A compound selected from the group consisting of:
(3aR,6aS)-N-{(1S)-3-Methyl-1-[6-oxo-4-(pyridine-2-carbonyl)-hexahydropyrrolo [3,2-b]pyrrole-1-carbonyl]-butyl}-benzamide;
(3aR,6aS)-N-{(1S)-3-Methyl-1-[6-oxo-4-(pyridine-2-carbonyl)-hexahydropyrrolo [3,2-c]pyrazole-1-carbonyl]-butyl}-benzamide;
(3aS,6aS)-N-{(1S)-3-Methyl-1-[6-oxo-4-(pyridine-2-carbonyl)-hexahydro-2-oxa-1,4-diaza-pentalene-1-carbonyl]-butyl}-benzamide.
Naphthalene-2-carboxylic acid {3-methyl-1-[6-oxo-4-(pyridine-2-sulfonyl)hexahydro-pyrrolo[3,2-b]pyrrole-1-carbonyl]-butyl}-amide;
Quinoline-2-carboxylic acid {3-methyl-1-[6-oxo-4-(pyridine-2-sulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
Benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
Biphenyl-4-carboxylic acid {3-methyl-1-[6-oxo-4-(pyridine-2-sulfonyl)-hexahydro-pyrrolo[3,2-b]pyrrole-1-carbonyl]-butyl}-amide;
4-tert-Butyl-N-{3-methyl-1-[6-oxo-4-(pyridine-2-sulfonyl)-hexahydro-pyrrolo[3,2-b]pyrrole-1-carbonyl]-butyl}-benzamide;
Dimethylamino-N-{3-methyl-1-[6-oxo-4-(pyridine-2-sulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
{3-Methyl-1-[6-oxo-4-(pyridine-2-sulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(pyridine-2-sulfonyl)-hexahydro-pyrrolo[3,2-b]pyrrole-1-carbonyl]-butyl}-amide;
Quinoxaline-2-carboxylic acid {3-methyl-1-[6-oxo-4-(pyridine-2-sulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(pyridine-2-sulfonyl)-hexahydro-pyrrolo[3,2-b]pyrrole-1-carbonyl]-butyl}-amide;
Quinoline-6-carboxylic acid {3-methyl-1-[6-oxo-4-(pyridine-2-sulfonyl)-hexahydro-pyrrolo[3,2-b]pyrrole-1-carbonyl]-butyl}-amide;
Furan-2-carboxylic acid {3-methyl-1-[6-oxo-4-(pyridine-2-sulfonyl)-hexahydro-pyrrolo[3,2-b]pyrrole-1-carbonyl]-butyl}-amide;
Thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
N-{3-Methyl-1-[6-oxo-4-(pyridine-2-sulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(pyridine-2-sulfonyl)-hexahydro-pyrrolo[3,2-b]pyrrole-1-carbonyl]-butyl}-amide;
Thiophene-3-carboxylic acid {3-methyl-1-[6-oxo-4-(pyridine-2-sulfonyl)-hexahydro-pyrrolo[3,2-b]pyrrole-1-carbonyl]-butyl}-amide;
N-{3-Methyl-1-[6-oxo-4-(pyridine-2-sulfonyl)-hexahydro-pyrrolo[3,2-b]pyrrole-1-carbonyl]-butyl}-4-phenoxy-benzamide;
N-{3-Methyl-1-[6-oxo-4-(pyridine-2-sulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(pyridine-2-sulfonyl)-hexahydro-pyrrolo[3,2-b]pyrrole-1-carbonyl]-butyl}-benzamide;
4-Methoxy-N-{3-methyl-1-[6-oxo-4-(pyridine-2-sulfonyl)-hexahydro-pyrrolo[3,2-b]pyrrole-1-carbonyl]-butyl}-benzamide;
4-Isopropyl-N-{3-methyl-1-[6-oxo-4-(pyridine-2-sulfonyl)-hexahydro-pyrrolo[3,2-b]pyrrole-1-carbonyl]-butyl}-benzamide;
N-{3-Methyl-1-[6-oxo-4-(pyridine-2-sulfonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(pyridine-2-sulfonyl)-hexahydro-pyrrolo[3,2-b]pyrrole-1-carbonyl]-butyl}-benzamide;
N-{3-Methyl-1-[6-oxo-4-(pyridine-2-sulfonyl)-hexahydro-pyrrolo[3,2-b]pyrrole-1-carbonyl]-butyl}-4-thiophen-2-yl-benzamide;
N-{3-Methyl-1-[6-oxo-4-(pyridine-2-sulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
N-{3-Methyl-1-[6-oxo-4-(pyridine-2-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(pyridine-2-sulfonyl)-hexahydro-pyrrolo[3,2-b]pyrrole-1-carbonyl]-butyl}-amide;
4-Difluoromethoxy-N-{3-methyl-1-[6-oxo-4-(pyridine-2-sulfonyl)-hexahydro-pyrrolo[3,2-b]pyrrole-1-carbonyl]-butyl}-benzamide;
N-{3-Methyl-1-[6-oxo-4-(pyridine-2-sulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(pyridine-2-sulfonyl)hexahydro-pyrrolo[3,2-b]pyrrole-1-carbonyl]-butyl}-amide;
Quinoline-2-carboxylic acid {1-[6-oxo-4-(pyridine-2-sulfonyl)-hexahydro-pyrrolo[3,2-b]pyrrole-1-carbonyl]-butyl}-amide;
Benzo[b]thiophene-2-carboxylic acid {1-[6-oxo-4-(pyridine-2-sulfonyl)-hexahydro-pyrrolo[3,2-b]pyrrole-1-carbonyl]-butyl}-amide;
Benzofuran-2-carboxylic acid 1-[6-oxo-4-(pyridine-2-sulfonyl)-hexahydro-pyrrolo[3,2-b]pyrrole-1-carbonyl]-butyl}-amide;
Biphenyl-4-carboxylic acid {1-[6-oxo-4-(pyridine-2-sulfonyl)-hexahydro-pyrrolo[3,2-b]pyrrole-1-carbonyl]-butyl}-amide;
4-tert-Butyl-N-{1-[6-oxo-4-(pyridine-2-sulfonyl)-hexahydro-pyrrolo[3,2-b]pyrrole-1-carbonyl]-butyl}-benzamide;
4-Dimethylamino-N-{1-[6-oxo-4-(pyridine-2-sulfonyl)-hexahydro-pyrrolo[3,2-b]pyrrole-1-carbonyl]-butyl}-benzamide;
7-Methoxy-benzofuran-2-carboxylic acid {1-[6-oxo-4-(pyridine-2-sulfonyl)-hexahydro-pyrrolo[3,2-b]pyrrole-1-carbonyl]-butyl}-amide;
{1-[6-oxo-4-(pyridine-2-sulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(pyridine-2-sulfonyl)-hexahydro-pyrrolo[3,2-b]pyrrole-1-carbonyl]-butyl}-amide;
Thieno[3,2-b]thiophene-2-carboxylic acid {1-[6-oxo-4-(pyridine-2-sulfonyl)-hexahydro-pyrrolo[3,2-b]pyrrole-1-carbonyl]-butyl}-amide;
3-Methyl-benzofuran-2-carboxylic acid {1-[6-oxo-4-(pyridine-2-sulfonyl)-hexahydro-pyrrolo[3,2-b]pyrrole-1-carbonyl]-butyl}-amide;
Quinoxaline-2-carboxylic acid {1-[6-oxo-4-(pyridine-2-sulfonyl)-hexahydro-pyrrolo[3,2-b]pyrrole-1-carbonyl]-butyl}-amide;
Benzo[1,3]dioxole-5-carboxylic acid {1-[6-oxo-4-(pyridine-2-sulfonyl)-hexahydro-pyrrolo[3,2-b]pyrrole-1-carbonyl]-butyl}-amide;
Quinoline-6-carboxylic acid {1-[6-oxo-4-(pyridine-2-sulfonyl)-hexahydro-pyrrolo[3,2-b]pyrrole-1-carbonyl]-butyl}-amide;
Furan-2-carboxylic acid {1-[6-oxo-4-(pyridine-2-sulfonyl)-hexahydro-pyrrolo[3,2-b]pyrrole-1-carbonyl]-butyl}-amide;
Thiophene-2-carboxylic acid {1-[6-oxo-4-(pyridine-2-sulfonyl)-hexahydro-pyrrolo[3,2-b]pyrrole-1-carbonyl]-butyl}-amide;
N-{1-[6-oxo-4-(pyridine-2-sulfonyl)-hexahydro-pyrrolo[3,2-b]pyrrole-1-carbonyl]-butyl}-4-trifluoromethyl-benzamide;
Furan-3-carboxylic acid {1-[6-oxo-4-(pyridine-2-sulfonyl)-hexahydro-pyrrolo[3,2-b]pyrrole-1-carbonyl]-butyl}-amide;
Thiophene-3-carboxylic acid {1-[6-oxo-4-(pyridine-2-sulfonyl)-hexahydro-pyrrolo[3,2-b]pyrrole-1-carbonyl]-butyl}-amide;
N-{1-[6-oxo-4-(pyridine-2-sulfonyl)-hexahydro-pyrrolo[3,2-b]pyrrole-1-carbonyl]-butyl}-4-phenoxy-benzamide;
N-{1-[6-oxo-4-(pyridine-2-sulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(pyridine-2-sulfonyl)-hexahydro-pyrrolo[3,2-b]pyrrole-1-carbonyl]-butyl}-benzamide;
4-Methoxy-N-{1-[6-oxo-4-(pyridine-2-sulfonyl)-hexahydro-pyrrolo[3,2-b]pyrrole-1-carbonyl]-butyl}-benzamide;
4-Isopropyl-N-{1-[6-oxo-4-(pyridine-2-sulfonyl)-hexahydro-pyrrolo[3,2-b]pyrrole-1-carbonyl]-butyl}-benzamide;
N-{1-[6-oxo-4-(pyridine-2-sulfonyl)-hexahydro-pyrrolo[3,2-b]pyrrole-1-carbonyl]-butyl}-4-vinyl-benzamide;
4-Imidazol-1-yl-N-{1-[6-oxo-4-(pyridine-2-sulfonyl)-hexahydro-pyrrolo[3,2-b]pyrrole-1-carbonyl]-butyl}-benzamide;
N-{1-[6-oxo-4-(pyridine-2-sulfonyl)-hexahydro-pyrrolo[3,2-b]pyrrole-1-carbonyl]-butyl}-4-thiophen-2-yl-benzamide;
N-{1-[6-oxo-4-(pyridine-2-sulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(pyridine-2-sulfonyl)-hexahydro-pyrrolo[3,2-b]pyrrole-1-carbonyl]-butyl}-amide;
N-{1-[6-oxo-4-(pyridine-2-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(pyridine-2-sulfonyl)-hexahydro-pyrrolo[3,2-b]pyrrole-1-carbonyl]-butyl}-amide;
4-Difluoromethoxy-N-{1-[6-oxo-4-(pyridine-2-sulfonyl)-hexahydro-pyrrolo[3,2-b]pyrrole-1-carbonyl]-butyl}-benzamide;
N-{1-[6-oxo-4-(pyridine-2-sulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(1-oxy-pyridine-2-sulfonyl)hexahydro-pyrrolo[3,2-b]pyrrole-1-carbonyl]-butyl}-amide;
Quinoline-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Biphenyl-4-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
4-tert-Butyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridine-2-sulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
{3-Methyl-1-[6-oxo-4-(1-oxy-pyridine-2-sulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Quinoxaline-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Quinoline-6-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Furan-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
N-{3-Methyl-1-[6-oxo-4-(1-oxy-pyridine-2-sulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
Thiophene-3-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
N-{3-Methyl-1-[6-oxo-4-(1-oxy-pyridine-2-sulfonyl)-hexahydro-pyrrolo[3,2-b]pyrrole-1-carbonyl]-butyl}-4-phenoxy-benzamide;
N-{3-Methyl-1-[6-oxo-4-(1-oxy-pyridine-2-sulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
4-Methoxy-N-{3-methyl-1-[6-oxo-4-(1-oxy-pyridine-2-sulfonyl)-hexahydro-pyrrolo[3,2-b]pyrrole-1-carbonyl]-butyl}-benzamide;
4-Isopropyl-N-{3-methyl-1-[6-oxo-4-(1-oxy-pyridine-2-sulfonyl)-hexahydro-pyrrolo[3,2-b]pyrrole-1-carbonyl]-butyl}-benzamide;
N-{3-Methyl-1-[6-oxo-4-(1-oxy-pyridine-2-sulfonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(1-oxy-pyridine-2-sulfonyl)-hexahydro-pyrrolo[3,2-b]pyrrole-1-carbonyl]-butyl}-benzamide;
N-{3-Methyl-1-[6-oxo-4-(1-oxy-pyridine-2-sulfonyl)-hexahydro-pyrrolo[3,2-b]pyrrole-1-carbonyl]-butyl}-4-thiophen-2-yl-benzamide;
N-{3-Methyl-1-[6-oxo-4-(1-oxy-pyridine-2-sulfonyl)-hexahydro-1 pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(1-oxy-pyridine-2-sulfonyl)-hexahydro-pyrrolo[3,2-b]pyrrole-1-carbonyl]-butyl}-amide;
N-{3-Methyl-1-[6-oxo-4-(1-oxy-pyridine-2-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(1-oxy-2 pyridine-2-sulfonyl)-hexahydro-pyrrolo[3,2-b]pyrrole-1-carbonyl]-butyl}-amide;
4-Difluoromethoxy-N-{3-methyl-1-[6-oxo-4-(1-oxy-pyridine-2-sulfonyl)-hexahydro-pyrrolo[3,2-b]pyrrole-1-carbonyl]-butyl}-benzamide;
N-{3-Methyl-1-[6-oxo-4-(1-oxy-pyridine-2-sulfonyl)-hexahy dro-pyrrolo[3,2-b]pyrrole-1-carbonyl]-butyl}-4-morpholin-4-yl-benzamide;
Naphthalene-2-carboxylic acid {1-[6-oxo-4-(1-oxy-pyridine-2-sulfonyl)hexahydro-pyrrolo[3,2-b]pyrrole-1-carbonyl]-butyl}-amide;
Quinoline-2-carboxylic acid {1-[6-oxo-4-(1-oxy-pyridine-2-sulfonyl)-hexahydro-pyrrolo[3,2-b]pyrrole-1-carbonyl]-butyl}-amide;
Benzo[b]thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
Benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
Biphenyl-4-carboxylic acid {1-[6-oxo-4-(1-oxy-pyridine-2-sulfonyl)-hexahydro-pyrrolo[3,2-b]pyrrole-1-carbonyl]-butyl}-amide;
4-tert-Butyl-N-{1-[6-oxo-4-(1-oxy-pyridine-2-sulfonyl)-hexahydro-pyrrolo[3,2-b]pyrrole-1-carbonyl]-butyl}-benzamide;
4-Dimethylamino-N-{1-[6-oxo-4-(1-oxy-pyridine-2-sulfonyl)-hexahydro-pyrrolo[3,2-b]pyrrole-1-carbonyl]-butyl}-benzamide;
7-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
{1-[6-oxo-4-(1-oxy-pyridine-2-sulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
Thieno[3,2-b]thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
3-Methyl-benzofuran-2-carboxylic acid {1-[6-oxo-4-(1-oxy-pyridine-2-sulfonyl)-hexahydro-pyrrolo[3,2-b]pyrrole-1-carbonyl]-butyl}-amide;
Quinoxaline-2-carboxylic acid {1-[6-oxo-4-(1-oxy-pyridine-2-sulfonyl)-hexahydro-pyrrolo[3,2-b]pyrrole-1-carbonyl]-butyl}-amide;
Benzo[1,3]dioxole-5-carboxylic acid {1-[6-oxo-4-(1-oxy-pyridine-2-sulfonyl)-hexahydro-pyrrolo[3,2-b]pyrrole-1-carbonyl]-butyl}-amide;
Quinoline-6-carboxylic acid {1-[6-oxo-4-(1-oxy-pyridine-2-sulfonyl)-hexahydro-pyrrolo[3,2-b]pyrrole-1-carbonyl]-butyl}-amide;
Furan-2-carboxylic acid {1-[6-oxo-4-(1-oxy-pyridine-2-sulfonyl)-hexahydro-pyrrolo[3,2-b]pyrrole-1-carbonyl]-butyl}-amide;
Thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
N-{1-[6-oxo-4-(1-oxy-pyridine-2-sulfonyl)-hexahydro-pyrrolo[3,2-b]pyrrole-1-carbonyl]-butyl}-4-trifluoromethyl-benzamide;
Furan-3-carboxylic acid {1-[6-oxo-4-(1-oxy-pyridine-2-sulfonyl)-hexahydro-pyrrolo[3,2-b]pyrrole-1-carbonyl]-butyl}-amide;
Thiophene-3-carboxylic acid {1-[6-oxo-4-(1-oxy-pyridine-2-sulfonyl)-hexahydro-pyrrolo[3,2-b]pyrrole-1-carbonyl]-butyl}-amide;
N-{1-[6-oxo-4-(1-oxy-pyridine-2-sulfonyl)-hexahydro-pyrrolo[3,2-b]pyrrole-1-carbonyl]-butyl}-4-phenoxy-benzamide;
N-{1-[6-oxo-4-(1-oxy-pyridine-2-sulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(1-oxy-pyridine-2-sulfonyl)-hexahydro-pyrrolo[3,2-b]pyrrole-1-carbonyl]-butyl}-benzamide;
4-Methoxy-N-{1-[6-oxo-4-(1-oxy-pyridine-2-sulfonyl)-hexahydro-pyrrolo[3,2-b]pyrrole-1-carbonyl]-butyl}-benzamide;
4-Isopropyl-N-{1-[6-oxo-4-(1-oxy-pyridine-2-sulfonyl)-hexahydro-pyrrolo[3,2-b]pyrrole-1-carbonyl]-butyl}-benzamide;
N-{1-[6-oxo-4-(1-oxy-pyridine-2-sulfonyl)-hexahydro-pyrrolo[3,2-b]pyrrole-1-carbonyl]-butyl}-4-vinyl-benzamide;
4-Imidazol-1-yl-N-{1-[6-oxo-4-(1-oxy-pyridine-2-sulfonyl)-hexahydro-pyrrolo[3,2-b]pyrrole-1-carbonyl]-butyl}-benzamide;
N-{1-[6-oxo-4-(1-oxy-pyridine-2-sulfonyl)-hexahydro-pyrrolo[3,2-b]pyrrole-1-carbonyl]-butyl}-4-thiophen-2-yl-benzamide;
N-{1-[6-oxo-4-(1-oxy-pyridine-2-sulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
N-{1-[6-oxo-4-(1-oxy-pyridine-2-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(1-oxy-pyridine-2-sulfonyl)-hexahydro-pyrrolo[3,2-b]pyrrole-1-carbonyl]-butyl}-amide;
4-Difluoromethoxy-N-{1-[6-oxo-4-(1-oxy-pyridine-2-sulfonyl)-3 hexahydro-pyrrolo[3,2-b]pyrrole-1-carbonyl]-butyl}-benzamide;
N-{1-[6-oxo-4-(1-oxy-pyridine-2-sulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(pyridine-3-sulfonyl)hexahydro-pyrrolo[3,2-b]pyrrole-1-carbonyl]-butyl}-amide;
Quinoline-2-carboxylic acid {3-methyl-1-[6-oxo-4-(pyridine-3-sulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
-573-Benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
Biphenyl-4-carboxylic acid {3-methyl-1-[6-oxo-4-(pyridine-3-sulfonyl)-hexahydro-pyrrolo[3,2-b]pyrrole-1-carbonyl]-butyl}-amide;
4-tert-Butyl-N-{3-methyl-1-[6-oxo-4-(pyridine-3-sulfonyl)-hexahy dro-pyrrolo[3,2-b]pyrrole-1-carbonyl]-butyl}-benzamide;
4-Dimethylamino-N-{3-methyl-1-[6-oxo-4-(pyridine-3-sulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
{3-Methyl-1-[6-oxo-4-(pyridine-3-sulfonyl)-hexahy dro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(pyridine-3-sulfonyl)-hexahydro-pyrrolo[3,2-b]pyrrole-1-carbonyl]-butyl}-amide;
Quinoxaline-2-carboxylic acid {3-methyl-1-[6-oxo-4-(pyridine-3-sulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(pyridine-3-sulfonyl)-hexahydro-pyrrolo[3,2-b]pyrrole-1-carbonyl]-butyl}-amide;
Quinoline-6-carboxylic acid {3-methyl-1-[6-oxo-4-(pyridine-3-sulfonyl)-hexahydro-pyrrolo[3,2-b]pyrrole-1-carbonyl]-butyl}-amide;
Furan-2-carboxylic acid {3-methyl-1-[6-oxo-4-(pyridine-3-sulfonyl)-hexahydro-pyrrolo[3,2-b]pyrrole-1-carbonyl]-butyl}-amide;
Thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
N-{3-Methyl-1-[6-oxo-4-(pyridine-3-sulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(pyridine-3-sulfonyl)-hexahydro-pyrrolo[3,2-b]pyrrole-1-carbonyl]-butyl}-amide;
Thiophene-3-carboxylic acid {3-methyl-1-[6-oxo-4-(pyridine-3-sulfonyl)-hexahydro-pyrrolo[3,2-b]pyrrole-1-carbonyl]-butyl}-amide;
N-{3-Methyl-1-[6-oxo-4-(pyridine-3-sulfonyl)-hexahydro-pyrrolo[3,2-b]pyrrole-1-carbonyl]-butyl}-4-phenoxy-benzamide;
N-{3-Methyl-1-[6-oxo-4-(pyridine-3-sulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(pyridine-3-sulfonyl)-hexahydro-pyrrolo[3,2-b]pyrrole-1-carbonyl]-butyl}-benzamide;
4-Methoxy-N-{3-methyl-1-[6-oxo-4-(pyridine-3-sulfonyl)-hexahydro-1 pyrrolo[3,2-b]pyrrole-1-carbonyl]-butyl}-benzamide;
4-Isopropyl-N-{3-methyl-1-[6-oxo-4-(pyridine-3-sulfonyl)-hexahydro-pyrrolo[3,2-b]pyrrole-1-carbonyl]-butyl}-benzamide;
N-{3-Methyl-1-[6-oxo-4-(pyridine-3-sulfonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(pyridine-3-sulfonyl)-hexahydro-pyrrolo[3,2-b]pyrrole-1-carbonyl]-butyl}-benzamide;
N-{3-Methyl-1-[6-oxo-4-(pyridine-3-sulfonyl)-hexahydro-pyrrolo[3,2-b]pyrrole-1-carbonyl]-butyl}-4-thiophen-2-yl-benzamide;
N-{3-Methyl-1-[6-oxo-4-(pyridine-3-sulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
N-{3-Methyl-1-[6-oxo-4-(pyridine-3-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(pyridine-3-sulfonyl)-hexahydro-pyrrolo[3,2-b]pyrrole-1-carbonyl]-butyl}-amide;
4-Difluoromethoxy-N-{3-methyl-1-[6-oxo-4-(pyridine-3-sulfonyl)-hexahydro-pyrrolo[3,2-b]pyrrole-1-carbonyl]-butyl}-benzamide;
N-{3-Methyl-1-[6-oxo-4-(pyridine-3-sulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(pyridine-3-sulfonyl)hexahydro-pyrrolo[3,2-b]pyrrole-1-carbonyl]-butyl}-amide;
Quinoline-2-carboxylic acid {1-[6-oxo-4-(pyridine-3-sulfonyl)-hexahydro-pyrrolo[3,2-b]pyrrole-1-carbonyl]-butyl}-amide;
Benzo[b]thiophene-2-carboxylic acid {1-[6-oxo-4-(pyridine-3-sulfonyl)-hexahydro-pyrrolo[3,2-b]pyrrole-1-carbonyl]-butyl}-amide;
Benzofuran-2-carboxylic acid {1-[6-oxo-4-(pyridine-3-sulfonyl)-hexahydro-pyrrolo[3,2-b]pyrrole-1-carbonyl]-butyl}-amide;
Biphenyl-4-carboxylic acid {1-[6-oxo-4-(pyridine-3-sulfonyl)-hexahydro-pyrrolo[3,2-b]pyrrole-1-carbonyl]-butyl}-amide;
4-tert-Butyl-N-{1-[6-oxo-4-(pyridine-3-sulfonyl)-hexahydro-pyrrolo[3,2-b]pyrrole-1-carbonyl]-butyl}-benzamide;
4-Dimethylamino-N-{1-[6-oxo-4-(pyridine-3-sulfonyl)-hexahydro-pyrrolo[3,2-b]pyrrole-1-carbonyl]-butyl}-benzamide;
7-Methoxy-benzofuran-2-carboxylic acid {1-[6-oxo-4-(pyridine-3-sulfonyl)-hexahydro-pyrrolo[3,2-b]pyrrole-1-carbonyl]-butyl}-amide;
{1-[6-oxo-4-(pyridine-3-sulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(pyridine-3-sulfonyl)-hexahydro-pyrrolo[3,2-b]pyrrole-1-carbonyl]-butyl}-amide;
Thieno[3,2-b]thiophene-2-carboxylic acid {1-[6-oxo-4-(pyridine-3-sulfonyl)-hexahydro-pyrrolo[3,2-b]pyrrole-1-carbonyl]-butyl}-amide;
3-Methyl-benzofuran-2-carboxylic acid {1-[6-oxo-4-(pyridine-3-sulfonyl)-hexahydro-pyrrolo[3,2-b]pyrrole-1-carbonyl]-butyl}-amide;
Quinoxaline-2-carboxylic acid {1-[6-oxo-4-(pyridine-3-sulfonyl)-hexahydro-pyrrolo[3,2-b]pyrrole-1-carbonyl]-butyl}-amide;
Benzo[1,3]dioxole-5-carboxylic acid {1-[6-oxo-4-(pyridine-3-sulfonyl)-hexahydro-pyrrolo[3,2-b]pyrrole-1-carbonyl]-butyl}-amide;
Quinoline-6-carboxylic acid {1-[6-oxo-4-(pyridine-3-sulfonyl)-hexahydro-pyrrolo[3,2-b]pyrrole-1-carbonyl]-butyl}-amide;
Furan-2-carboxylic acid {1-[6-oxo-4-(pyridine-3-sulfonyl)-hexahydro-pyrrolo[3,2-b]pyrrole-1-carbonyl]-butyl}-amide;
Thiophene-2-carboxylic acid {1-[6-oxo-4-(pyridine-3-sulfonyl)-hexahydro-pyrrolo[3,2-b]pyrrole-1-carbonyl]-butyl}-amide;
N-{1-[6-oxo-4-(pyridine-3-sulfonyl)-hexahydro-pyrrolo[3,2-b]pyrrole-1-carbonyl]-butyl}-4-trifluoromethyl-benzamide;
Furan-3-carboxylic acid {1-[6-oxo-4-(pyridine-3-sulfonyl)-hexahydro-pyrrolo[3,2-b]pyrrole-1-carbonyl]-butyl}-amide;
Thiophene-3-carboxylic acid {1-[6-oxo-4-(pyridine-3-sulfonyl)-hexahydro-pyrrolo[3,2-b]pyrrole-1-carbonyl]-butyl}-amide;
N-{1-[6-oxo-4-(pyridine-3-sulfonyl)-hexahydro-pyrrolo[3,2-b]pyrrole-1-carbonyl]-butyl}-4-phenoxy-benzamide;
N-{1-[6-oxo-4-(pyridine-3-sulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(pyridine-3-sulfonyl)-hexahydro-pyrrolo[3,2-b]pyrrole-1-carbonyl]-butyl}-benzamide;
4-Methoxy-N-{1-[6-oxo-4-(pyridine-3-sulfonyl)-hexahydro-pyrrolo[3,2-b]pyrrole-1-carbonyl]-butyl}-benzamide;
4-Isopropyl-N-{1-[6-oxo-4-(pyridine-3-sulfonyl)-hexahydro-pyrrolo[3,2-b]pyrrole-1-carbonyl]-butyl}-benzamide;
N-{-[6-oxo-4-(pyridine-3-sulfonyl)-hexahydro-pyrrolo[3,2-b]pyrrole-1-carbonyl]-butyl}-4-vinyl-benzamide;
4-Imidazol-1-yl-N-{1-[6-oxo-4-(pyridine-3-sulfonyl)-hexahydro-pyrrolo[3,2-b]pyrrole-1-carbonyl]-butyl}-benzamide;
N-{1-[6-oxo-4-(pyridine-3-sulfonyl)-hexahydro-pyrrolo[3,2-b]pyrrole-1-3 carbonyl]-butyl}-4-thiophen-2-yl-benzamide;
N-{1-[6-oxo-4-(pyridine-3-sulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(pyridine-3-sulfonyl)-hexahydro-pyrrolo[3,2-b]pyrrole-1-carbonyl]-butyl}-amide;
N-{1-[6-oxo-4-(pyridine-3-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(pyridine-3-sulfonyl)-hexahydro-pyrrolo[3,2-b]pyrrole-1-carbonyl]-butyl}-amide;
4-Difluoromethoxy-N-{1-[6-oxo-4-(pyridine-3-sulfonyl)-hexahydro-pyrrolo[3,2-b]pyrrole-1-carbonyl]-butyl}-benzamide;
N-{1-[6-oxo-4-(pyridine-3-sulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(1-oxy-pyridine-3-sulfonyl)hexahydro-pyrrolo[3,2-b]pyrrole-1-carbonyl]-butyl}-amide;
Quinoline-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Biphenyl-4-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
4-tert-Butyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridine-3-sulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
{3-Methyl-1-[6-oxo-4-(1-oxy-pyridine-3-sulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Quinoxaline-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Quinoline-6-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Furan-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
N-{3-Methyl-1-[6-oxo-4-(1-oxy-pyridine-3-sulfonyl)-hexahy dro-pyrrolo[3,2-b]pyrrole-1-carbonyl]-butyl}-4-trifluoromethyl-benzamide;
Furan-3-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
Thiophene-3-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
N-{3-Methyl-1-[6-oxo-4-(1-oxy-pyridine-3-sulfonyl)-hexahy dro-pyrrolo[3,2-b]pyrrole-1-carbonyl]-butyl}-4-phenoxy-benzamide;
N-{3-Methyl-1-[6-oxo-4-(1-oxy-pyridine-3-sulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
4-Methoxy-N-{3-methyl-1-[6-oxo-4-(1-oxy-pyridine-3-sulfonyl)-hexahydro-pyrrolo[3,2-b]pyrrole-1-carbonyl]-butyl}-benzamide;
4-Isopropyl-N-{3-methyl-1-[6-oxo-4-(1-oxy-pyridine-3-sulfonyl)-hexahydro-pyrrolo[3,2-b]pyrrole-1-carbonyl]-butyl}-benzamide;
N-{3-Methyl-1-[6-oxo-4-(1-oxy-pyridine-3-sulfonyl)-hexahy dro-pyrrolo[3,2-b]pyrrole-1-carbonyl]-butyl}-4-vinyl-benzamide;
4-Imidazol-1-yl-N-{3-methyl-1-[6-oxo-4-(1-oxy-pyridine-3-sulfonyl)-hexahydro-pyrrolo[3,2-b]pyrrole-1-carbonyl]-butyl}-benzamide;
N-{3-Methyl-1-[6-oxo-4-(1-oxy-pyridine-3-sulfonyl)-hexahy dro-pyrrolo[3,2-b]pyrrole-1-carbonyl]-butyl}-4-thiophen-2-yl-benzamide;
N-{3-Methyl-1-[6-oxo-4-(1-oxy-pyridine-3-sulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(1-oxy-pyridine-3-sulfonyl)-hexahydro-pyrrolo[3,2-b]pyrrole-1-carbonyl]-butyl}-amide;
N-{3-Methyl-1-[6-oxo-4-(1-oxy-pyridine-3-sulfonyl)-hexahy dro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(1-oxy-1 pyridine-3-sulfonyl)-hexahydro-pyrrolo[3,2-b]pyrrole-1-carbonyl]-butyl}-amide;
4-Difluoromethoxy-N-{3-methyl-1-[6-oxo-4-(1-oxy-pyridine-3-sulfonyl)-hexahydro-pyrrolo[3,2-b]pyrrole-1-carbonyl]-butyl}-benzamide;
N-{3-Methyl-1-[6-oxo-4-(1-oxy-pyridine-3-sulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(1-oxy-pyridine-3-sulfonyl)hexahydro-pyrrolo[3,2-b]pyrrole-1-carbonyl]-butyl}-amide;
Quinoline-2-carboxylic acid {1-[6-oxo-4-(1-oxy-pyridine-3-sulfonyl)-hexahydro-pyrrolo[3,2-b]pyrrole-1-carbonyl]-butyl}-amide;
Benzo[b]thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
Benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
Biphenyl-4-carboxylic acid {1-[6-oxo-4-(1-oxy-pyridine-3-sulfonyl)-hexahydro-pyrrolo[3,2-b]pyrrole-1-carbonyl]-butyl}-amide;
4-tert-Butyl-N-{1-[6-oxo-4-(1-oxy-pyridine-3-sulfonyl)-hexahydro-pyrrolo[3,2-b]pyrrole-1-carbonyl]-butyl}-benzamide;
4-Dimethylamino-N-{1-[6-oxo-4-(1-oxy-pyridine-3-sulfonyl)-hexahydro-pyrrolo[3,2-b]pyrrole-1-carbonyl]-butyl}-benzamide;
7-Methoxy-benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
{1-[6-oxo-4-(1-oxy-pyridine-3-sulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
Thieno[3,2-b]thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
3-Methyl-benzofuran-2-carboxylic acid {1-[6-oxo-4-(1-oxy-pyridine-3-sulfonyl)-hexahydro-pyrrolo[3,2-b]pyrrole-1-carbonyl]-butyl}-amide;
Quinoxaline-2-carboxylic acid {1-[6-oxo-4-(1-oxy-pyridine-3-sulfonyl)-hexahydro-pyrrolo[3,2-b]pyrrole-1-carbonyl]-butyl}-amide;
Benzo[1,3]dioxole-5-carboxylic acid {1-[6-oxo-4-(1-oxy-pyridine-3-sulfonyl)-hexahydro-pyrrolo[3,2-b]pyrrole-1-carbonyl]-butyl}-amide;
Quinoline-6-carboxylic acid {1-[6-oxo-4-(1-oxy-pyridine-3-sulfonyl)-hexahydro-pyrrolo[3,2-b]pyrrole-1-carbonyl]-butyl}-amide;
Furan-2-carboxylic acid {1-[6-oxo-4-(1-oxy-pyridine-3-sulfonyl)-hexahydro-pyrrolo[3,2-b]pyrrole-1-carbonyl]-butyl}-amide;
Thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
N-{1-[6-oxo-4-(1-oxy-pyridine-3-sulfonyl)-hexahydro-pyrrolo[3,2-b]pyrrole-1-carbonyl]-butyl}-4-trifluoromethyl-benzamide;
Furan-3-carboxylic acid {1-[6-oxo-4-(1-oxy-pyridine-3-sulfonyl)-hexahydro-pyrrolo[3,2-b]pyrrole-1-carbonyl]-butyl}-amide;
Thiophene-3-carboxylic acid {1-[6-oxo-4-(1-oxy-pyridine-3-sulfonyl)-hexahydro-pyrrolo[3,2-b]pyrrole-1-carbonyl]-butyl}-amide;
N-{1-[6-oxo-4-(1-oxy-pyridine-3-sulfonyl)-hexahydro-pyrrolo[3,2-b]pyrrole-1-carbonyl]-butyl}-4-phenoxy-benzamide;
N-{1-[6-oxo-4-(1-oxy-pyridine-3-sulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(1-oxy-pyridine-3-sulfonyl)-hexahydro-pyrrolo[3,2-b]pyrrole-1-carbonyl]-butyl}-benzamide;
4-Methoxy-N-{1-[6-oxo-4-(1-oxy-pyridine-3-sulfonyl)-hexahydro-pyrrolo[3,2-b]pyrrole-1-carbonyl]-butyl}-benzamide;
4-Isopropyl-N-{1-[6-oxo-4-(1-oxy-pyridine-3-sulfonyl)-hexahydro-pyrrolo[3,2-b]pyrrole-1-carbonyl]-butyl}-benzamide;
N-{1-[6-oxo-4-(1-oxy-pyridine-3-sulfonyl)-hexahydro-pyrrolo[3,2-b]pyrrole-1-carbonyl]-butyl}-4-vinyl-benzamide;
4-Imidazol-1-yl-N-{1-[6-oxo-4-(1-oxy-pyridine-3-sulfonyl)-hexahydro-pyrrolo[3,2-b]pyrrole-1-carbonyl]-butyl}-benzamide;
N-{1-[6-oxo-4-(1-oxy-pyridine-3-sulfonyl)-hexahydro-pyrrolo[3,2-b]pyrrole-1-carbonyl]-butyl}-4-thiophen-2-yl-benzamide;
N-{1-[6-oxo-4-(1-oxy-pyridine-3-sulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
N-{1-[6-oxo-4-(1-oxy-pyridine-3-sulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(1-oxy-pyridine-3-sulfonyl)-hexahydro-pyrrolo[3,2-b]pyrrole-1-carbonyl]-butyl}-amide;
4-Difluoromethoxy-N-{1-[6-oxo-4-(1-oxy-pyridine-3-sulfonyl)-hexahydro-pyrrolo[3,2-b]pyrrole-1-carbonyl]-butyl}-benzamide;
N-{1-[6-oxo-4-(1-oxy-pyridine-3-sulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(pyridine-2-carbonyl)hexahydro-pyrrolo[3,2-b]pyrrole-1-carbonyl]-butyl}-amide;
Quinoline-2-carboxylic acid {3-methyl-1-[6-oxo-4-(pyridine-2-carbonyl)-hexahydro-pyrrolo[3,2-b]pyrrole-1-carbonyl]-butyl}-amide;
Benzo[b]thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
Benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
Biphenyl-4-carboxylic acid {3-methyl-1-[6-oxo-4-(pyridine-2-carbonyl)-hexahydro-pyrrolo[3,2-b]pyrrole-1-carbonyl]-butyl}-amide;
4-tert-Butyl-N-{3-methyl-1-[6-oxo-4-(pyridine-2-carbonyl)-hexahy dro-pyrrolo[3,2-b]pyrrole-1-carbonyl]-butyl}-benzamide;
4-Dimethylamino-N-{3-methyl-1-[6-oxo-4-(pyridine-2-carbonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
{3-Methyl-1-[6-oxo-4-(pyridine-2-carbonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
3-Methyl-benzofuran-2-carboxylic acid {3-methyl-1-[6-oxo-4-(pyridine-2-carbonyl)-hexahydro-pyrrolo[3,2-b]pyrrole-1-carbonyl]-butyl}-amide;
Quinoxaline-2-carboxylic acid {3-methyl-1-[6-oxo-4-(pyridine-2-carbonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(pyridine-2-carbonyl)-hexahydro-pyrrolo[3,2-b]pyrrole-1-carbonyl]-butyl}-amide;
Quinoline-6-carboxylic acid {3-methyl-1-[6-oxo-4-(pyridine-2-carbonyl)-hexahydro-pyrrolo[3,2-b]pyrrole-1-carbonyl]-butyl}-amide;
Furan-2-carboxylic acid {3-methyl-1-[6-oxo-4-(pyridine-2-carbonyl)-hexahydro-pyrrolo[3,2-b]pyrrole-1-carbonyl]-butyl}-amide;
Thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
N-{3-Methyl-1-[6-oxo-4-(pyridine-2-carbonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(pyridine-2-carbonyl)-hexahydro-pyrrolo[3,2-b]pyrrole-1-carbonyl]-butyl}-amide;
Thiophene-3-carboxylic acid {3-methyl-1-[6-oxo-4-(pyridine-2-carbonyl)-hexahydro-pyrrolo[3,2-b]pyrrole-1-carbonyl]-butyl}-amide;
N-{3-Methyl-1-[6-oxo-4-(pyridine-2-carbonyl)-hexahydro-pyrrolo[3,2-b]pyrrole-1-carbonyl]-butyl}-4-phenoxy-benzamide;
N-{3-Methyl-1-[6-oxo-4-(pyridine-2-carbonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(pyridine-2-carbonyl)-hexahydro-pyrrolo[3,2-b]pyrrole-1-carbonyl]-butyl}-benzamide;
4-Methoxy-N-{3-methyl-1-[6-oxo-4-(pyridine-2-carbonyl)-hexahydro-pyrrolo[3,2-b]pyrrole-1-carbonyl]-butyl}-benzamide;
4-Isopropyl-N-{3-methyl-1-[6-oxo-4-(pyridine-2-carbonyl)-hexahydro-pyrrolo[3,2-b]pyrrole-1-carbonyl]-butyl}-benzamide;
N-{3-Methyl-1-[6-oxo-4-(pyridine-2-carbonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(pyridine-2-carbonyl)-1 hexahydro-pyrrolo[3,2-b]pyrrole-1-carbonyl]-butyl}-benzamide;
N-{3-Methyl-1-[6-oxo-4-(pyridine-2-carbonyl)-hexahydro-pyrrolo[3,2-b]pyrrole-1-carbonyl]-butyl}-4-thiophen-2-yl-benzamide;
N-{3-Methyl-1-[6-oxo-4-(pyridine-2-carbonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
N-{3-Methyl-1-[6-oxo-4-(pyridine-2-carbonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(pyridine-2-carbonyl)-hexahydro-pyrrolo[3,2-b]pyrrole-1-carbonyl]-butyl}-amide;
4-Difluoromethoxy-N-{3-methyl-1-[6-oxo-4-(pyridine-2-carbonyl)-hexahydro-pyrrolo[3,2-b]pyrrole-1-carbonyl]-butyl}-benzamide;
N-{3-Methyl-1-[6-oxo-4-(pyridine-2-carbonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(pyridine-2-carbonyl)hexahydro-pyrrolo[3,2-b]pyrrole-1-carbonyl]-butyl}-amide;
Quinoline-2-carboxylic acid {1-[6-oxo-4-(pyridine-2-carbonyl)-hexahydro-pyrrolo[3,2-b]pyrrole-1-carbonyl]-butyl}-amide;
Benzo[b]thiophene-2-carboxylic acid {1-[6-oxo-4-(pyridine-2-carbonyl)-hexahydro-pyrrolo[3,2-b]pyrrole-1-carbonyl]-butyl}-amide;
Benzofuran-2-carboxylic acid {1-[6-oxo-4-(pyridine-2-carbonyl)-hexahydro-pyrrolo[3,2-b]pyrrole-1-carbonyl]-butyl}-amide;
Biphenyl-4-carboxylic acid {1-[6-oxo-4-(pyridine-2-carbonyl)-hexahydro-pyrrolo[3,2-b]pyrrole-1-carbonyl]-butyl}-amide;
4-tert-Butyl-N-{1-[6-oxo-4-(pyridine-2-carbonyl)-hexahydro-pyrrolo[3,2-b]pyrrole-1-carbonyl]-butyl}-benzamide;
4-Dimethylamino-N-{1-[6-oxo-4-(pyridine-2-carbonyl)-hexahydro-pyrrolo[3,2-b]pyrrole-1-carbonyl]-butyl}-benzamide;
7-Methoxy-benzofuran-2-carboxylic acid {1-[6-oxo-4-(pyridine-2-carbonyl)-hexahydro-pyrrolo[3,2-b]pyrrole-1-carbonyl]-butyl}-amide;
{1-[6-oxo-4-(pyridine-2-carbonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(pyridine-2-carbonyl)-hexahydro-pyrrolo[3,2-b]pyrrole-1-carbonyl]-butyl}-amide;
Thieno[3,2-b]thiophene-2-carboxylic acid {1-[6-oxo-4-(pyridine-2-carbonyl)-hexahydro-pyrrolo[3,2-b]pyrrole-1-carbonyl]-butyl}-amide;
3-Methyl-benzofuran-2-carboxylic acid {1-[6-oxo-4-(pyridine-2-carbonyl)-hexahydro-pyrrolo[3,2-b]pyrrole-1-carbonyl]-butyl}-amide;
Quinoxaline-2-carboxylic acid {1-[6-oxo-4-(pyridine-2-carbonyl)-hexahydro-pyrrolo[3,2-b]pyrrole-1-carbonyl]-butyl}-amide;
Benzo[1,3]dioxole-5-carboxylic acid {1-[6-oxo-4-(pyridine-2-carbonyl)-hexahydro-pyrrolo[3,2-b]pyrrole-1-carbonyl]-butyl}-amide;
Quinoline-6-carboxylic acid {1-[6-oxo-4-(pyridine-2-carbonyl)-hexahydro-pyrrolo[3,2-b]pyrrole-1-carbonyl]-butyl}-amide;
Furan-2-carboxylic acid {1-[6-oxo-4-(pyridine-2-carbonyl)-hexahydro-pyrrolo[3,2-b]pyrrole-1-carbonyl]-butyl}-amide;
Thiophene-2-carboxylic acid {1-[6-oxo-4-(pyridine-2-carbonyl)-hexahydro-pyrrolo[3,2-b]pyrrole-1-carbonyl]-butyl}-amide;
N-{1-[6-oxo-4-(pyridine-2-carbonyl)-hexahydro-pyrrolo[3,2-b]pyrrole-1-carbonyl]-butyl}-4-trifluoromethyl-benzamide;
Furan-3-carboxylic acid {1-[6-oxo-4-(pyridine-2-carbonyl)-hexahydro-pyrrolo[3,2-b]pyrrole-1-carbonyl]-butyl}-amide;
Thiophene-3-carboxylic acid {1-[6-oxo-4-(pyridine-2-carbonyl)-hexahydro-pyrrolo[3,2-b]pyrrole-1-carbonyl]-butyl}-amide;
N-{1-[6-oxo-4-(pyridine-2-carbonyl)-hexahydro-pyrrolo[3,2-b]pyrrole-1-carbonyl]-butyl}-4-phenoxy-benzamide;
N-{1-[6-oxo-4-(pyridine-2-carbonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(pyridine-2-carbonyl)-hexahydro-pyrrolo[3,2-b]pyrrole-1-carbonyl]-butyl}-benzamide;
4-Methoxy-N-{1-[6-oxo-4-(pyridine-2-carbonyl)-hexahydro-pyrrolo[3,2-b]pyrrole-1-carbonyl]-butyl}-benzamide;
4-Isopropyl-N-{1-[6-oxo-4-(pyridine-2-carbonyl)-hexahydro-pyrrolo[3,2-b]pyrrole-1-carbonyl]-butyl}-benzamide;
N-{1-[6-oxo-4-(pyridine-2-carbonyl)-hexahydro-pyrrolo[3,2-b]pyrrole-1-carbonyl]-butyl}-4-vinyl-benzamide;
4-Imidazol-1-yl-N-{1-[6-oxo-4-(pyridine-2-carbonyl)-hexahydro-pyrrolo[3,2-b]pyrrole-1-carbonyl]-butyl}-benzamide;
N-{1-[6-oxo-4-(pyridine-2-carbonyl)-hexahydro-pyrrolo[3,2-b]pyrrole-1-carbonyl]-butyl}-4-thiophen-2-yl-benzamide;
N-{1-[6-oxo-4-(pyridine-2-carbonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(pyridine-2-carbonyl)-hexahydro-pyrrolo[3,2-b]pyrrole-1-carbonyl]-butyl}-amide;
N-{1-[6-oxo-4-(pyridine-2-carbonyl)-hexahydro-pyrrolo[3,2-b]pyrrole-1-3 carbonyl]-butyl}-4-trifluoromethoxy-benzamide;
5-Pyridin-2-yl-thiophene-2-carboxylic acid {1-[6-oxo-4-(pyridine-2-carbonyl)-hexahydro-pyrrolo[3,2-b]pyrrole-1-carbonyl]-butyl}-amide;
4-Difluoromethoxy-N-{1-[6-oxo-4-(pyridine-2-carbonyl)-hexahydro-pyrrolo[3,2-b]pyrrole-1-carbonyl]-butyl}-benzamide;
N-{1-[6-oxo-4-(pyridine-2-carbonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(2-pyridin-3-yl-acetyl)hexahydro-pyrrolo[3,2-b]pyrrole-1-carbonyl]-butyl}-amide;
Quinoline-2-carboxylic acid {3-methyl-1-[6-oxo-4-(2-pyri din-3-yl-acetyl)-hexahydro-pyrrolo[3,2-b]pyrrole-1-carbonyl]-butyl}-amide;
Benzo[b]thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Biphenyl-4-carboxylic acid {3-methyl-1-[6-oxo-4-(2-pyridin-3-yl-acetyl)-hexahydro-pyrrolo[3,2-b]pyrrole-1-carbonyl]-butyl}-amide;
4-tert-Butyl-N-{3-methyl-1-[6-oxo-4-(2-pyridin-3-yl-acetyl)-hexahydro-pyrrolo[3,2-b]pyrrole-1-carbonyl]-butyl}-benzamide;
4-Dimethylamino-N-{3-methyl-1-[6-oxo-4-(2-pyri din-3-yl-acetyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
{3-Methyl-1-[6-oxo-4-(2-pyridin-3-yl-acetyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
3-Methyl-benzofuran-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Quinoxaline-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Benzo[1,3]dioxole-5-carboxylic acid {3-methyl-1-[6-oxo-4-(2-pyri din-3-yl-acetyl)-hexahydro-pyrrolo[3,2-b]pyrrole-1-carbonyl]-butyl}-amide;
Quinoline-6-carboxylic acid {3-methyl-1-[6-oxo-4-(2-pyridin-3-yl-acetyl)-hexahydro-pyrrolo[3,2-b]pyrrole-1-carbonyl]-butyl}-amide;
Furan-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
Thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
N-{3-Methyl-1-[6-oxo-4-(2-pyridin-3-yl-acetyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(2-pyridin-3-yl-acetyl)-hexahydro-pyrrolo[3,2-b]pyrrole-1-carbonyl]-butyl}-amide;
Thiophene-3-carboxylic acid {3-methyl-1-[6-oxo-4-(2-pyridin-3-yl-acetyl)-hexahydro-pyrrolo[3,2-b]pyrrole-1-carbonyl]-butyl}-amide;
N-{3-Methyl-1-[6-oxo-4-(2-pyridin-3-yl-acetyl)-hexahydro-pyrrolo[3,2-b]pyrrole-1-carbonyl]-butyl}-4-phenoxy-benzamide;
N-{3-Methyl-1-[6-oxo-4-(2-pyridin-3-yl-acetyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(2-pyridin-3-yl-acetyl)-hexahydro-pyrrolo[3,2-b]pyrrole-1-carbonyl]-butyl}-benzamide;
4-Methoxy-N-{3-methyl-1-[6-oxo-4-(2-pyridin-3-yl-acetyl)-hexahydro-pyrrolo[3,2-b]pyrrole-1-carbonyl]-butyl}-benzamide;
4-Isopropyl-N-{3-methyl-1-[6-oxo-4-(2-pyridin-3-yl-acetyl)-hexahydro-pyrrolo[3,2-b]pyrrole-1-carbonyl]-butyl}-benzamide;
N-{3-Methyl-1-[6-oxo-4-(2-pyridin-3-yl-acetyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(2-pyridin-3-yl-acetyl)-hexahydro-pyrrolo[3,2-b]pyrrole-1-carbonyl]-butyl}-benzamide;
N-{3-Methyl-1-[6-oxo-4-(2-pyridin-3-yl-acetyl)-hexahydro-pyrrolo[3,2-b]pyrrole-1-carbonyl]-butyl}-4-thiophen-2-yl-benzamide;
N-{3-Methyl-1-[6-oxo-4-(2-pyridin-3-yl-acetyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
N-{3-Methyl-1-[6-oxo-4-(2-pyridin-3-yl-acetyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(2-pyridin-3-yl-acetyl)-hexahydro-pyrrolo[3,2-b]pyrrole-1-carbonyl]-butyl}-amide;
4-Difluoromethoxy-N-{3-methyl-1-[6-oxo-4-(2-pyridin-3-yl-acetyl)-hexahydro-pyrrolo[3,2-b]pyrrole-1-carbonyl]-butyl}-benzamide;
N-{3-Methyl-1-[6-oxo-4-(2-pyridin-3-yl-acetyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(2-pyridin-3-yl-acetyl)hexahydro-pyrrolo[3,2-b]pyrrole-1-carbonyl]-butyl}-amide;
Quinoline-2-carboxylic acid {1-[6-oxo-4-(2-pyridin-3-yl-acetyl)-hexahydro-pyrrolo[3,2-b]pyrrole-1-carbonyl]-butyl}-amide;
Benzo[b]thiophene-2-carboxylic acid {1-[6-oxo-4-(2-pyridin-3-yl-acetyl)-1-hexahydro-pyrrolo[3,2-b]pyrrole-1-carbonyl]-butyl}-amide;
Benzofuran-2-carboxylic acid {1-[6-oxo-4-(2-pyridin-3-yl-acetyl)-hexahydro-pyrrolo[3,2-b]pyrrole-1-carbonyl]-butyl}-amide;
Biphenyl-4-carboxylic acid {1-[6-oxo-4-(2-pyridin-3-yl-acetyl)-hexahydro-pyrrolo[3,2-b]pyrrole-1-carbonyl]-butyl}-amide;
4-tert-Butyl-N-{1-[6-oxo-4-(2-pyridin-3-yl-acetyl)-hexahydro-pyrrolo[3,2-b]pyrrole-1-carbonyl]-butyl}-benzamide;
4-Dimethylamino-N-{1-[6-oxo-4-(2-pyridin-3-yl-acetyl)-hexahydro-pyrrolo[3,2-b]pyrrole-1-carbonyl]-butyl}-benzamide;
7-Methoxy-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
{1-[6-oxo-4-(2-pyridin-3-yl-acetyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
Thieno[3,2-b]thiophene-2-carboxylic acid {1-[6-oxo-4-(2-pyridin-3-yl-acetyl)-hexahydro-pyrrolo[3,2-b]pyrrole-1-carbonyl]-butyl}-amide;
3-Methyl-benzofuran-2-carboxylic acid {1-[6-oxo-4-(2-pyridin-3-yl-acetyl)-hexahydro-pyrrolo[3,2-b]pyrrole-1-carbonyl]-butyl}-amide;
Quinoxaline-2-carboxylic acid {1-[6-oxo-4-(2-pyridin-3-yl-acetyl)-4 hexahydro-pyrrolo[3,2-b]pyrrole-1-carbonyl]-butyl}-amide;
Benzo[1,3]dioxole-5-carboxylic acid {1-[6-oxo-4-(2-pyridin-3-yl-acetyl)-hexahydro-pyrrolo[3,2-b]pyrrole-1-carbonyl]-butyl}-amide;
Quinoline-6-carboxylic acid {1-[6-oxo-4-(2-pyridin-3-yl-acetyl)-hexahydro-pyrrolo[3,2-b]pyrrole-1-carbonyl]-butyl}-amide;
Furan-2-carboxylic acid {1-[6-oxo-4-(2-pyridin-3-yl-acetyl)-hexahydro-pyrrolo[3,2-b]pyrrole-1-carbonyl]-butyl}-amide;
Thiophene-2-carboxylic acid {1-[6-oxo-4-(2-pyridin-3-yl-acetyl)-hexahydro-pyrrolo[3,2-b]pyrrole-1-carbonyl]-butyl}-amide;
N-{1-[6-oxo-4-(2-pyridin-3-yl-acetyl)-hexahydro-pyrrolo[3,2-b]pyrrole-1-carbonyl]-butyl}-4-trifluoromethyl-benzamide;
Furan-3-carboxylic acid {1-[6-oxo-4-(2-pyridin-3-yl-acetyl)-hexahydro-1 pyrrolo[3,2-b]pyrrole-1-carbonyl]-butyl}-amide;
Thiophene-3-carboxylic acid {1-[6-oxo-4-(2-pyridin-3-yl-acetyl)-hexahydro-pyrrolo[3,2-b]pyrrole-1-carbonyl]-butyl}-amide;
N-{1-[6-oxo-4-(2-pyridin-3-yl-acetyl)-hexahydro-pyrrolo[3,2-b]pyrrole-1-carbonyl]-butyl}-4-phenoxy-benzamide;
N-{1-[6-oxo-4-(2-pyridin-3-yl-acetyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(2-pyridin-3-yl-acetyl)-hexahydro-pyrrolo[3,2-b]pyrrole-1-carbonyl]-butyl}-benzamide;
4-Methoxy-N-{1-[6-oxo-4-(2-pyridin-3-yl-acetyl)-hexahydro-pyrrolo[3,2-b]pyrrole-1-carbonyl]-butyl}-benzamide;
4-Isopropyl-N-{1-[6-oxo-4-(2-pyridin-3-yl-acetyl)-hexahydro-pyrrolo[3,2-b]pyrrole-1-carbonyl]-butyl}-benzamide;
N-{1-[6-oxo-4-(2-pyridin-3-yl-acetyl)-hexahydro-pyrrolo[3,2-b]pyrrole-1-carbonyl]-butyl}-4-vinyl-benzamide;
4-Imidazol-1-yl-N-{1-[6-oxo-4-(2-pyridin-3-yl-acetyl)-hexahydro-pyrrolo[3,2-b]pyrrole-1-carbonyl]-butyl}-benzamide;
N-{1-[6-oxo-4-(2-pyridin-3-yl-acetyl)-hexahydro-pyrrolo[3,2-b]pyrrole-1-carbonyl]-butyl}-4-thiophen-2-yl-benzamide;
N-{1-[6-oxo-4-(2-pyridin-3-yl-acetyl)-hexahydro-pyrrolo[3,2-b]pyrrole-1-4-carbonyl]-butyl}-4-oxazol-5-yl-benzamide;
5-Phenyl-thiophene-2-carboxylic acid {1-[6-oxo-4-(2-pyridin-3-yl-acetyl)-hexahydro-pyrrolo[3,2-b]pyrrole-1-carbonyl]-butyl}-amide;
N-{1-[6-oxo-4-(2-pyridin-3-yl-acetyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(2-pyridin-3-yl-acetyl)-hexahydro-pyrrolo[3,2-b]pyrrole-1-carbonyl]-butyl}-amide;
4-Difluoromethoxy-N-{1-[6-oxo-4-(2-pyridin-3-yl-acetyl)-hexahydro-pyrrolo[3,2-b]pyrrole-1-carbonyl]-butyl}-benzamide;
N-{1-[6-oxo-4-(2-pyridin-3-yl-acetyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(pyridin-2-ylmethanesulfonyl)hexahydro-pyrrolo[3,2-b]pyrrole-1-carbonyl]-butyl}-amide;
Quinoline-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Biphenyl-4-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
4-tert-Butyl-N-{3-methyl-1-[6-oxo-4-(pyri din-2-ylmethanesulfonyl)-3 hexahydro-pyrrolo[3,2-b]pyrrole-1-carbonyl]-butyl}-benzamide;
4-Dimethylamino-N-{3-methyl-1-[6-oxo-4-(pyridin-2-ylmethanesulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
{3-Methyl-1-[6-oxo-4-(pyridin-2-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Quinoxaline-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Quinoline-6-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Furan-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
N-{3-Methyl-1-[6-oxo-4-(pyridin-2-ylmethanesulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
Thiophene-3-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
N-{3-Methyl-1-[6-oxo-4-(pyridin-2-ylmethanesulfonyl)-hexahydro-pyrrolo[3,2-b]pyrrole-1-carbonyl]-butyl}-4-phenoxy-benzamide;
N-{3-Methyl-1-[6-oxo-4-(pyridin-2-ylmethanesulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(pyridin-2-ylmethanesulfonyl)-hexahydro-pyrrolo[3,2-b]pyrrole-1-carbonyl]-butyl}-benzamide;
4-Methoxy-N-{3-methyl-1-[6-oxo-4-(pyridin-2-ylmethanesulfonyl)-hexahydro-pyrrolo[3,2-b]pyrrole-1-carbonyl]-butyl}-benzamide;
4-Isopropyl-N-{3-methyl-1-[6-oxo-4-(pyridin-2-ylmethanesulfonyl)-hexahydro-pyrrolo[3,2-b]pyrrole-1-carbonyl]-butyl}-benzamide;
N-{3-Methyl-1-[6-oxo-4-(pyridin-2-ylmethanesulfonyl)-hexahydro-1 pyrrolo[3,2-b]pyrrole-1-carbonyl]-butyl}-4-vinyl-benzamide;
4-Imidazol-1-yl-N-{3-methyl-1-[6-oxo-4-(pyridin-2-ylmethanesulfonyl)-hexahydro-pyrrolo[3,2-b]pyrrole-1-carbonyl]-butyl}-benzamide;
N-{3-Methyl-1-[6-oxo-4-(pyridin-2-ylmethanesulfonyl)-hexahydro-pyrrolo[3,2-b]pyrrole-1-carbonyl]-butyl}-4-thiophen-2-yl-benzamide;
N-{3-Methyl-1-[6-oxo-4-(pyridin-2-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
N-{3-Methyl-1-[6-oxo-4-(pyridin-2-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(pyridin-2-ylmethanesulfonyl)-hexahydro-pyrrolo[3,2-b]pyrrole-1-carbonyl]-butyl}-amide;
4-Difluoromethoxy-N-{3-methyl-1-[6-oxo-4-(pyridin-2-ylmethanesulfonyl)-hexahydro-pyrrolo[3,2-b]pyrrole-1-carbonyl]-butyl}-benzamide;
N-{3-Methyl-1-[6-oxo-4-(pyridin-2-ylmethanesulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(pyridin-2-ylmethanesulfonyl)hexahydro-pyrrolo[3,2-b]pyrrole-1-carbonyl]-butyl}-amide;
Quinoline-2-carboxylic acid {1-[6-oxo-4-(pyridin-2-ylmethanesulfonyl)-hexahydro-pyrrolo[3,2-b]pyrrole-1-carbonyl]-butyl}-amide;
Benzo[b]thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
Benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
Biphenyl-4-carboxylic acid {1-[6-oxo-4-(pyridin-2-ylmethanesulfonyl)-hexahydro-pyrrolo[3,2-b]pyrrole-1-carbonyl]-butyl}-amide;
4-tert-Butyl-N-{1-[6-oxo-4-(pyridin-2-ylmethanesulfonyl)-hexahydro-1 pyrrolo[3,2-b]pyrrole-1-carbonyl]-butyl}-benzamide;
4-Dimethylamino-N-{1-[6-oxo-4-(pyridin-2-ylmethanesulfonyl)-hexahydro-pyrrolo[3,2-b]pyrrole-1-carbonyl]-butyl}-benzamide;
7-Methoxy-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
{1-[6-oxo-4-(pyridin-2-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
Thieno[3,2-b]thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
3-Methyl-benzofuran-2-carboxylic acid {1-[6-oxo-4-(pyridin-2-ylmethanesulfonyl)-hexahydro-pyrrolo[3,2-b]pyrrole-1-carbonyl]-butyl}-amide;
Quinoxaline-2-carboxylic acid {1-[6-oxo-4-(pyridin-2-ylmethanesulfonyl)-hexahydro-pyrrolo[3,2-b]pyrrole-1-carbonyl]-butyl}-amide;
Benzo[1,3]dioxole-5-carboxylic acid {1-[6-oxo-4-(pyridin-2-ylmethanesulfonyl)-hexahydro-pyrrolo[3,2-b]pyrrole-1-carbonyl]-butyl}-amide;
Quinoline-6-carboxylic acid {1-[6-oxo-4-(pyridin-2-ylmethanesulfonyl)-hexahydro-pyrrolo[3,2-b]pyrrole-1-carbonyl]-butyl}-amide;
Furan-2-carboxylic acid {1-[6-oxo-4-(pyridin-2-ylmethanesulfonyl)-hexahydro-pyrrolo[3,2-b]pyrrole-1-carbonyl]-butyl}-amide;
Thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
N-{1-[6-oxo-4-(pyridin-2-ylmethanesulfonyl)-hexahydro-pyrrolo[3,2-b]pyrrole-1-carbonyl]-butyl}-4-trifluoromethyl-benzamide;
Furan-3-carboxylic acid {1-[6-oxo-4-(pyridin-2-ylmethanesulfonyl)-hexahydro-pyrrolo[3,2-b]pyrrole-1-carbonyl]-butyl}-amide;
Thiophene-3-carboxylic acid {1-[6-oxo-4-(pyridin-2-ylmethanesulfonyl)-1 hexahydro-pyrrolo[3,2-b]pyrrole-1-carbonyl]-butyl}-amide;
N-{1-[6-oxo-4-(pyridin-2-ylmethanesulfonyl)-hexahydro-pyrrolo[3,2-b]pyrrole-1-carbonyl]-butyl}-4-phenoxy-benzamide;
N-{1-[6-oxo-4-(pyridin-2-ylmethanesulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(pyridin-2-ylmethanesulfonyl)-hexahydro-pyrrolo[3,2-b]pyrrole-1-carbonyl]-butyl}-benzamide;
4-Methoxy-N-{1-[6-oxo-4-(pyridin-2-ylmethanesulfonyl)-hexahydro-pyrrolo[3,2-b]pyrrole-1-carbonyl]-butyl}-benzamide;
4-Isopropyl-N-{1-[6-oxo-4-(pyridin-2-ylmethanesulfonyl)-hexahydro-pyrrolo[3,2-b]pyrrole-1-carbonyl]-butyl}-benzamide;
N-{1-[6-oxo-4-(pyridin-2-ylmethanesulfonyl)-hexahydro-pyrrolo[3,2-b]pyrrole-1-carbonyl]-butyl}-4-vinyl-benzamide;
4-Imidazol-1-yl-N-{1-[6-oxo-4-(pyridin-2-ylmethanesulfonyl)-hexahydro-pyrrolo[3,2-b]pyrrole-1-carbonyl]-butyl}-benzamide;
N-{1-[6-oxo-4-(pyridin-2-ylmethanesulfonyl)-hexahydro-pyrrolo[3,2-b]pyrrole-1-carbonyl]-butyl}-4-thiophen-2-yl-benzamide;
N-{1-[6-oxo-4-(pyridin-2-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
N-{1-[6-oxo-4-(pyridin-2-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(pyridin-2-ylmethanesulfonyl)-hexahydro-pyrrolo[3,2-b]pyrrole-1-carbonyl]-butyl}-amide;
4-Difluoromethoxy-N-{1-[6-oxo-4-(pyridin-2-ylmethanesulfonyl)-hexahydro-pyrrolo[3,2-b]pyrrole-1-carbonyl]-butyl}-benzamide;
N-{1-[6-oxo-4-(pyridin-2-ylmethanesulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(pyridin-3-ylmethanesulfonyl)hexahydro-pyrrolo[3,2-b]pyrrole-1-carbonyl]-butyl}-amide;
Quinoline-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Biphenyl-4-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
4-tert-Butyl-N-{3-methyl-1-[6-oxo-4-(pyri din-3-ylmethanesulfonyl)-3 hexahydro-pyrrolo[3,2-b]pyrrole-1-carbonyl]-butyl}-benzamide;
4-Dimethylamino-N-{3-methyl-1-[6-oxo-4-(pyridin-3-ylmethanesulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
{3-Methyl-1-[6-oxo-4-(pyridin-3-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Quinoxaline-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Quinoline-6-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Furan-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
N-{3-Methyl-1-[6-oxo-4-(pyridin-3-ylmethanesulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
Thiophene-3-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
N-{3-Methyl-1-[6-oxo-4-(pyridin-3-ylmethanesulfonyl)-hexahydro-pyrrolo[3,2-b]pyrrole-1-carbonyl]-butyl}-4-phenoxy-benzamide;
N-{3-Methyl-1-[6-oxo-4-(pyridin-3-ylmethanesulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(pyridin-3-ylmethanesulfonyl)-hexahydro-pyrrolo[3,2-b]pyrrole-1-carbonyl]-butyl}-benzamide;
4-Methoxy-N-{3-methyl-1-[6-oxo-4-(pyridin-3-ylmethanesulfonyl)-hexahydro-pyrrolo[3,2-b]pyrrole-1-carbonyl]-butyl}-benzamide;
4-Isopropyl-N-{3-methyl-1-[6-oxo-4-(pyridin-3-ylmethanesulfonyl)-hexahydro-pyrrolo[3,2-b]pyrrole-1-carbonyl]-butyl}-benzamide;
N-{3-Methyl-1-[6-oxo-4-(pyridin-3-ylmethanesulfonyl)-hexahydro-1 pyrrolo[3,2-b]pyrrole-1-carbonyl]-butyl}-4-vinyl-benzamide;
4-Imidazol-1-yl-N-{3-methyl-1-[6-oxo-4-(pyridin-3-ylmethanesulfonyl)-hexahydro-pyrrolo[3,2-b]pyrrole-1-carbonyl]-butyl}-benzamide;
N-{3-Methyl-1-[6-oxo-4-(pyridin-3-ylmethanesulfonyl)-hexahydro-pyrrolo[3,2-b]pyrrole-1-carbonyl]-butyl}-4-thiophen-2-yl-benzamide;
N-{3-Methyl-1-[6-oxo-4-(pyridin-3-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
N-{3-Methyl-1-[6-oxo-4-(pyridin-3-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(pyridin-3-ylmethanesulfonyl)-hexahydro-pyrrolo[3,2-b]pyrrole-1-carbonyl]-butyl}-amide;
4-Difluoromethoxy-N-{3-methyl-1-[6-oxo-4-(pyridin-3-ylmethanesulfonyl)-hexahydro-pyrrolo[3,2-b]pyrrole-1-carbonyl]-butyl}-benzamide;
N-{3-Methyl-1-[6-oxo-4-(pyridin-3-ylmethanesulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(pyridin-3-ylmethanesulfonyl)hexahydro-pyrrolo[3,2-b]pyrrole-1-carbonyl]-butyl}-amide;
Quinoline-2-carboxylic acid {1-[6-oxo-4-(pyridin-3-ylmethanesulfonyl)-hexahydro-pyrrolo[3,2-b]pyrrole-1-carbonyl]-butyl}-amide;
Benzo[b]thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
Benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
Biphenyl-4-carboxylic acid {1-[6-oxo-4-(pyridin-3-ylmethanesulfonyl)-hexahydro-pyrrolo[3,2-b]pyrrole-1-carbonyl]-butyl}-amide;
4-tert-Butyl-N-{1-[6-oxo-4-(pyridin-3-ylmethanesulfonyl)-hexahydro-1 pyrrolo[3,2-b]pyrrole-1-carbonyl]-butyl}-benzamide;
4-Dimethylamino-N-{1-[6-oxo-4-(pyridin-3-ylmethanesulfonyl)-hexahydro-pyrrolo[3,2-b]pyrrole-1-carbonyl]-butyl}-benzamide;
7-Methoxy-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
{1-[6-oxo-4-(pyridin-3-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
Thieno[3,2-b]thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
3-Methyl-benzofuran-2-carboxylic acid {1-[6-oxo-4-(pyridin-3-ylmethanesulfonyl)-hexahydro-pyrrolo[3,2-b]pyrrole-1-carbonyl]-butyl}-amide;
Quinoxaline-2-carboxylic acid {1-[6-oxo-4-(pyridin-3-ylmethanesulfonyl)-hexahydro-pyrrolo[3,2-b]pyrrole-1-carbonyl]-butyl}-amide;
Benzo[1,3]dioxole-5-carboxylic acid {1-[6-oxo-4-(pyridin-3-ylmethanesulfonyl)-hexahydro-pyrrolo[3,2-b]pyrrole-1-carbonyl]-butyl}-amide;
Quinoline-6-carboxylic acid {1-[6-oxo-4-(pyridin-3-ylmethanesulfonyl)-hexahydro-pyrrolo[3,2-b]pyrrole-1-carbonyl]-butyl}-amide;
Furan-2-carboxylic acid {1-[6-oxo-4-(pyridin-3-ylmethanesulfonyl)-hexahydro-pyrrolo[3,2-b]pyrrole-1-carbonyl]-butyl}-amide;
Thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
N-{1-[6-oxo-4-(pyridin-3-ylmethanesulfonyl)-hexahydro-pyrrolo[3,2-b]pyrrole-1-carbonyl]-butyl}-4-trifluoromethyl-benzamide;
Furan-3-carboxylic acid {1-[6-oxo-4-(pyridin-3-ylmethanesulfonyl)-hexahydro-pyrrolo[3,2-b]pyrrole-1-carbonyl]-butyl}-amide;
Thiophene-3-carboxylic acid {1-[6-oxo-4-(pyridin-3-ylmethanesulfonyl)-1 hexahydro-pyrrolo[3,2-b]pyrrole-1-carbonyl]-butyl}-amide;
N-{1-[6-oxo-4-(pyridin-3-ylmethanesulfonyl)-hexahydro-pyrrolo[3,2-b]pyrrole-1-carbonyl]-butyl}-4-phenoxy-benzamide;
N-{1-[6-oxo-4-(pyridin-3-ylmethanesulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(pyridin-3-ylmethanesulfonyl)-hexahydro-pyrrolo[3,2-b]pyrrole-1-carbonyl]-butyl}-benzamide;
4-Methoxy-N-{1-[6-oxo-4-(pyridin-3-ylmethanesulfonyl)-hexahydro-pyrrolo[3,2-b]pyrrole-1-carbonyl]-butyl}-benzamide;
4-Isopropyl-N-{1-[6-oxo-4-(pyridin-3-ylmethanesulfonyl)-hexahydro-pyrrolo[3,2-b]pyrrole-1-carbonyl]-butyl}-benzamide;
N-{1-[6-oxo-4-(pyridin-3-ylmethanesulfonyl)-hexahydro-pyrrolo[3,2-b]pyrrole-1-carbonyl]-butyl}-4-vinyl-benzamide;
4-Imidazol-1-yl-N-{1-[6-oxo-4-(pyridin-3-ylmethanesulfonyl)-hexahydro-pyrrolo[3,2-b]pyrrole-1-carbonyl]-butyl}-benzamide;
N-{1-[6-oxo-4-(pyridin-3-ylmethanesulfonyl)-hexahydro-pyrrolo[3,2-b]pyrrole-1-carbonyl]-butyl}-4-thiophen-2-yl-benzamide;
N-{1-[6-oxo-4-(pyridin-3-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
N-{1-[6-oxo-4-(pyridin-3-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(pyridin-3-ylmethanesulfonyl)-hexahydro-pyrrolo[3,2-b]pyrrole-1-carbonyl]-butyl}-amide;
4-Difluoromethoxy-N-{1-[6-oxo-4-(pyridin-3-ylmethanesulfonyl)-hexahydro-pyrrolo[3,2-b]pyrrole-1-carbonyl]-butyl}-benzamide;
N-{1-[6-oxo-4-(pyridin-3-ylmethanesulfonyl)-hexahydro-pyrrolo[3,2-b]pyrrole-1-carbonyl]-butyl}-4-morpholin-4-yl-benzamide;
Naphthalene-2-carboxylic acid {3-methyl-1-[6-oxo-4-(1-oxy-pyridin-2-1 ylmethanesulfonyl)hexahydro-pyrrolo[3,2-b]pyrrole-1-carbonyl]-butyl}-amide;
Quinoline-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Biphenyl-4-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
4-tert-Butyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
{3-Methyl-1-[6-oxo-4-(1-oxy-pyridin-2-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Quinoxaline-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Quinoline-6-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Furan-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-trifluoromethyl-benzamide;
Furan-3-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
Thiophene-3-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-phenoxy-benzamide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
4-Methoxy-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
4-Isopropyl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-thiophen-2-yl-benzamide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-2-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(1-oxy-pyridin-2-ylmethanesulfonyl)-hexahydro-pyrrolo[3,2-b]pyrrole-1-carbonyl]-butyl}-amide;
4-Difluoromethoxy-N-{3-methyl-1-[6-oxo-4-(1-oxy-pyridin-2-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-2-ylmethanesulfonyl)-hexahy dro-pyrrolo[3,2-b]pyrrole-1-carbonyl]-butyl}-4-morpholin-4-yl-benzamide;
Naphthalene-2-carboxylic acid {1-[6-oxo-4-(1-oxy-pyridin-2-ylmethanesulfonyl)hexahydro-pyrrolo[3,2-b]pyrrole-1-carbonyl]-butyl}-amide;
Quinoline-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Benzo[b]thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Biphenyl-4-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
4-tert-Butyl-N-{1-[6-oxo-4-(1-oxy-pyridin-2-ylmethanesulfonyl)-hexahydro-pyrrolo[3,2-b]pyrrole-1-carbonyl]-butyl}-benzamide;
4-Dimethylamino-N-{1-[6-oxo-4-(1-oxy-pyridin-2-ylmethanesulfonyl)-2 hexahydro-pyrrolo[3,2-b]pyrrole-1-carbonyl]-butyl}-benzamide;
7-Methoxy-benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
{1-[6-oxo-4-(1-oxy-pyridin-2-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(1-oxy-pyridin-2-3 ylmethanesulfonyl)-hexahydro-pyrrolo[3,2-b]pyrrole-1-carbonyl]-butyl}-amide;
Thieno[3,2-b]thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
3-Methyl-benzofuran-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Quinoxaline-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Benzo[1,3]dioxole-5-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Quinoline-6-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Furan-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
N-{1-[6-oxo-4-(1-oxy-pyridin-2-ylmethanesulfonyl)-hexahydro-pyrrolo[3,2-b]pyrrole-1-carbonyl]-butyl}-4-trifluoromethyl-benzamide;
Furan-3-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
Thiophene-3-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
N-{1-[6-oxo-4-(1-oxy-pyridin-2-ylmethanesulfonyl)-hexahydro-pyrrolo[3,2-b]pyrrole-1-carbonyl]-butyl}-4-phenoxy-benzamide;
N-{1-[6-oxo-4-(1-oxy-pyridin-2-ylmethanesulfonyl)-hexahydro-pyrrolo[3,2-b]pyrrole-1-carbonyl]-butyl}-4-(4-methyl-piperazin-1-yl)-benzamide;
4-Methyl-N-{1-[6-oxo-4-(1-oxy-pyridin-2-ylmethanesulfonyl)-hexahydro-pyrrolo[3,2-b]pyrrole-1-carbonyl]-butyl}-benzamide;
4-Methoxy-N-{1-[6-oxo-4-(1-oxy-pyridin-2-ylmethanesulfonyl)-hexahydro-pyrrolo[3,2-b]pyrrole-1-carbonyl]-butyl}-benzamide;
4-Isopropyl-N-{1-[6-oxo-4-(1-oxy-pyridin-2-ylmethanesulfonyl)-hexahydro-pyrrolo[3,2-b]pyrrole-1-carbonyl]-butyl}-benzamide;
N-{1-[6-oxo-4-(1-oxy-pyridin-2-ylmethanesulfonyl)-hexahydro-pyrrolo[3,2-b]pyrrole-1-carbonyl]-butyl}-4-vinyl-benzamide;
4-Imidazol-1-yl-N-{1-[6-oxo-4-(1-oxy-pyridin-2-ylmethanesulfonyl)-hexahydro-pyrrolo[3,2-b]pyrrole-1-carbonyl]-butyl}-benzamide;
N-{1-[6-oxo-4-(1-oxy-pyridin-2-ylmethanesulfonyl)-hexahydro-pyrrolo[3,2-b]pyrrole-1-carbonyl]-butyl}-4-thiophen-2-yl-benzamide;
N-{1-[6-oxo-4-(1-oxy-pyridin-2-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
N-{1-[6-oxo-4-(1-oxy-pyridin-2-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(1-oxy-pyridin-2-ylmethanesulfonyl)-hexahydro-pyrrolo[3,2-b]pyrrole-1-carbonyl]-butyl}-amide;
4-Difluoromethoxy-N-{1-[6-oxo-4-(1-oxy-pyridin-2-ylmethanesulfonyl)-hexahydro-pyrrolo[3,2-b]pyrrole-1-carbonyl]-butyl}-benzamide;
N-{1-[6-oxo-4-(1-oxy-pyridin-2-ylmethanesulfonyl)-hexahydro-2 pyrrolo[3,2-b]pyrrole-1-carbonyl]-butyl}-4-morpholin-4-yl-benzamide;
Naphthalene-2-carboxylic acid {3-methyl-1-[6-oxo-4-(1-oxy-pyridin-3-ylmethanesulfonyl)hexahydro-pyrrolo[3,2-b]pyrrole-1-carbonyl]-butyl}-amide;
Quinoline-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Benzo[b]thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Biphenyl-4-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
4-tert-Butyl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
7-Methoxy-benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
{3-Methyl-1-[6-oxo-4-(1-oxy-pyridin-3-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Thieno[3,2-b]thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
3-Methyl-benzofuran-2-carboxylic acid {3-methyl-1-[6-oxo-4-(1-oxy-2 pyridin-3-ylmethanesulfonyl)-hexahydro-pyrrolo[3,2-b]pyrrole-1-carbonyl]-butyl}-amide;
Quinoxaline-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Benzo[1,3]dioxole-5-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Quinoline-6-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Furan-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-3-ylmethanesulfonyl)-hexahy dro-pyrrolo[3,2-b]pyrrole-1-carbonyl]-butyl}-4-trifluoromethyl-benzamide;
Furan-3-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
Thiophene-3-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-phenoxy-benzamide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-(4-methyl-piperazin-1-yl)-benzamide;
4-Methyl-N-{3-methyl-1-[6-oxo-4-(1-oxy-pyridin-3-ylmethanesulfonyl)-hexahydro-pyrrolo[3,2-b]pyrrole-1-carbonyl]-butyl 3-benzamide;
4-Methoxy-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
4-Isopropyl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-vinyl-benzamide;
4-Imidazol-1-yl-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-3-ylmethanesulfonyl)-hexahy dro-pyrrolo[3,2-b]pyrrole-1-carbonyl]-butyl}-4-thiophen-2-yl-benzamide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {3-methyl-1-[6-oxo-4-(1-oxy-pyridin-3-ylmethanesulfonyl)-hexahydro-pyrrolo[3,2-b]pyrrole-1-carbonyl]-butyl}-amide;
4-Difluoromethoxy-N-{3-methyl-1-[6-oxo-4-(1-oxy-pyridin-3-ylmethanesulfonyl)-hexahydro-pyrrolo[3,2-b]pyrrole-1-carbonyl]-butyl}-benzamide;
N-{3-Methyl-1-[6-oxo-4-(1-oxy-pyridin-3-ylmethanesulfonyl)-hexahydro-pyrrolo[3,2-b]pyrrole-1-carbonyl]-butyl}-4-morpholin-4-yl-benzamide;
Naphthalene-2-carboxylic acid {1-[6-oxo-4-(1-oxy-pyridin-3-ylmethanesulfonyl)hexahydro-pyrrolo[3,2-b]pyrrole-1-carbonyl]-butyl}-amide;
Quinoline-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Benzo[b]thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Biphenyl-4-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
4-tert-Butyl-N-{1-[6-oxo-4-(1-oxy-pyridin-3-ylmethanesulfonyl)-hexahydro-pyrrolo[3,2-b]pyrrole-1-carbonyl]-butyl}-benzamide;
4-Dimethylamino-N-{1-[6-oxo-4-(1-oxy-pyridin-3-ylmethanesulfonyl)-hexahydro-pyrrolo[3,2-b]pyrrole-1-carbonyl]-butyl}-benzamide;
7-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
{1-[6-oxo-4-(1-oxy-pyridin-3-ylmethanesulfonyl)-hexahydro-pyrrolo[3,2-b]pyrrole-1-carbonyl]-butyl}-carbamic acid benzyl ester;
5-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Thieno[3,2-b]thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
3-Methyl-benzofuran-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Quinoxaline-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Benzo[1,3]dioxole-5-carboxylic acid {1-[6-oxo-4-(1-oxy-pyridin-3-1 ylmethanesulfonyl)-hexahydro-pyrrolo[3,2-b]pyrrole-1-carbonyl]-butyl}-amide;
Quinoline-6-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Furan-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
N-{1-[6-oxo-4-(1-oxy-pyridin-3-ylmethanesulfonyl)-hexahydro-pyrrolo[3,2-b]pyrrole-1-carbonyl]-butyl}-4-trifluoromethyl-benzamide;
Furan-3-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
Thiophene-3-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
N-{1-[6-oxo-4-(1-oxy-pyridin-3-ylmethanesulfonyl)-hexahydro-pyrrolo[3,2-b]pyrrole-1-carbonyl]-butyl}-4-phenoxy-benzamide;
N-{1-[6-oxo-4-(1-oxy-pyridin-3-ylmethanesulfonyl)-hexahydro-4 pyrrolo[3,2-b]pyrrole-1-carbonyl]-butyl}-4-(4-methyl-piperazin-1-yl)-benzamide;
4-Methyl-N-{1-[6-oxo-4-(1-oxy-pyridin-3-ylmethanesulfonyl)-hexahydro-pyrrolo[3,2-b]pyrrole-1-carbonyl]-butyl}-benzamide;
4-Methoxy-N-{1-[6-oxo-4-(1-oxy-pyridin-3-ylmethanesulfonyl)-hexahydro-pyrrolo[3,2-b]pyrrole-1-carbonyl]-butyl}-benzamide;
4-Isopropyl-N-{1-[6-oxo-4-(1-oxy-pyridin-3-ylmethanesulfonyl)-hexahydro-pyrrolo[3,2-b]pyrrole-1-carbonyl]-butyl}-benzamide;
N-{1-[6-oxo-4-(1-oxy-pyridin-3-ylmethanesulfonyl)-hexahydro-pyrrolo[3,2-b]pyrrole-1-carbonyl]-butyl}-4-vinyl-benzamide;
4-Imidazol-1-yl-N-{1-[6-oxo-4-(1-oxy-pyridin-3-ylmethanesulfonyl)-hexahydro-pyrrolo[3,2-b]pyrrole-1-carbonyl]-butyl}-benzamide;
N-{1-[6-oxo-4-(1-oxy-pyridin-3-ylmethanesulfonyl)-hexahydro-pyrrolo[3,2-b]pyrrole-1-carbonyl]-butyl}-4-thiophen-2-yl-benzamide;
N-{1-[6-oxo-4-(1-oxy-pyridin-3-ylmethanesulfonyl)-hexahydro-pyrrolo[3,2-b]pyrrole-1-carbonyl]-butyl}-4-oxazol-5-yl-benzamide;
5-Phenyl-thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
N-{1-[6-oxo-4-(1-oxy-pyridin-3-ylmethanesulfonyl)-hexahydro-pyrrolo[3,2-b]pyrrole-1-carbonyl]-butyl}-4-trifluoromethoxy-benzamide;
5-Pyridin-2-yl-thiophene-2-carboxylic acid {1-[6-oxo-4-(1-oxy-pyridin-3-ylmethanesulfonyl)-hexahydro-pyrrolo[3,2-b]pyrrole-1-carbonyl]-butyl}-amide;
4-Difluoromethoxy-N-{1-[6-oxo-4-(1-oxy-pyridin-3-ylmethanesulfonyl)-hexahydro-pyrrolo[3,2-b]pyrrole-1-carbonyl]-butyl}-benzamide;
N-{1-[6-oxo-4-(1-oxy-pyridin-3-ylmethanesulfonyl)-hexahydro-pyrrolo[3,2-b]pyrrole-1-carbonyl]-butyl}-4-morpholin-4-yl-benzamide;
4-tert-Butyl-N-{1-[6-oxo-4-(pyridine-2-sulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(pyridine-2-sulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(pyridine-2-sulfonyl)-hexahydro-pyrrolo[3,2-b]pyrrole-1-carbonyl]-cyclohexyl}-amide;
3-Methyl-benzofuran-2-carboxylic acid {1-[6-oxo-4-(pyridine-2-sulfonyl)-hexahydro-pyrrolo[3,2-b]pyrrole-1-carbonyl]-cyclohexyl}-amide;
N-{1-[6-oxo-4-(pyridine-2-sulfonyl)-hexahydro-pyrrolo[3,2-b]pyrrole-1-carbonyl]-cyclohexyl}-4-phenoxy-benzamide;
N-{1-[6-oxo-4-(pyridine-2-sulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(pyridine-2-sulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(pyridine-2-sulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(pyridine-2-sulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(pyridine-2-sulfonyl)-hexahydro-pyrrolo[3,2-b]pyrrole-1-yl]-ethyl}-amide;
N-{1-cyclopropylmethyl-2-oxo-2-[6-oxo-4-(pyridine-2-sulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(pyridine-2-sulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(pyridine-2-sulfonyl)-hexahydro-pyrrolo[3,2-b]pyrrol-1-yl]-1-thiophen-2-ylmethyl-ethyl}-benzamide;
5-Methoxy-benzofuran-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(pyridine-2-sulfonyl)-hexahydro-pyrrolo[3,2-b]pyrrole-1-yl]-1-thiophen-2-ylmethyl-ethyl}-amide;
N-{2-oxo-2-[6-oxo-4-(pyridine-2-sulfonyl)-hexahydro-pyrrolo[3,2-b]pyrrole-1-yl]-1-thiophen-2-ylmethyl-ethyl}-4-phenoxy-benzamide;
N-{2-oxo-2-[6-oxo-4-(pyridine-2-sulfonyl)-hexahydro-pyrrolo[3,2-b]pyrrole-1-yl]-1-thiophen-2-ylmethyl-ethyl}-4-thiophen-2-yl-benzamide;
4-tert-Butyl-N-{1-[6-oxo-4-(pyridine-3-sulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(pyridine-3-sulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(pyridine-3-sulfonyl)-hexahydro-pyrrolo[3,2-b]pyrrole-1-carbonyl]-cyclohexyl}-amide;
3-Methyl-benzofuran-2-carboxylic acid {1-[6-oxo-4-(pyridine-3-sulfonyl)-hexahydro-pyrrolo[3,2-b]pyrrole-1-carbonyl]-cyclohexyl}-amide;
N-{1-[6-oxo-4-(pyridine-3-sulfonyl)-hexahydro-pyrrolo[3,2-b]pyrrole-1-carbonyl]-cyclohexyl}-4-phenoxy-benzamide;
N-{1-[6-oxo-4-(pyridine-3-sulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(pyridine-3-sulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(pyridine-3-sulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(pyridine-3-sulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(pyridine-3-sulfonyl)-hexahydro-pyrrolo[3,2-b]pyrrole-1-yl]-ethyl}-amide;
N-{1-cyclopropylmethyl-2-oxo-2-[6-oxo-4-(pyridine-3-sulfonyl)-hexahydro-pyrrolo [3,2-b]pyrrole-1-yl]-ethyl}-4-phenoxy-benzamide;
N-{1-cyclopropylmethyl-2-oxo-2-[6-oxo-4-(pyridine-3-sulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(pyridine-3-sulfonyl)-hexahydro-pyrrolo[3,2-b]pyrrol-1-yl]-1-thiophen-2-ylmethyl-ethyl}-benzamide;
5-Methoxy-benzofuran-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(pyridine-3-sulfonyl)-hexahydro-pyrrolo[3,2-b]pyrrole-1-yl]-1-thiophen-2-ylmethyl-ethyl}-amide;
N-{2-oxo-2-[6-oxo-4-(pyridine-3-sulfonyl)-hexahydro-pyrrolo[3,2-b]pyrrole-1-yl]-1-thiophen-2-ylmethyl-ethyl}-4-phenoxy-benzamide;
N-{2-oxo-2-[6-oxo-4-(pyridine-3-sulfonyl)-hexahydro-pyrrolo[3,2-b]pyrrole-1-yl]-1-thiophen-2-ylmethyl-ethyl}-4-thiophen-2-yl-benzamide;
4-tert-Butyl-N-{1-[6-oxo-4-(1-oxy-pyridine-2-sulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
3-Methyl-benzofuran-2-carboxylic acid {1-[6-oxo-4-(1-oxy-pyridine-2-sulfonyl)-hexahydro-pyrrolo[3,2-b]pyrrole-1-carbonyl]-cyclohexyl}-amide;
N-{1-[6-oxo-4-(1-oxy-pyridine-2-sulfonyl)-hexahydro-pyrrolo[3,2-b]pyrrole-1-carbonyl]-cyclohexyl}-4-phenoxy-benzamide;
N-{1-[6-oxo-4-(1-oxy-pyridine-2-sulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(1-oxy-pyridine-3 2-sulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(1-oxy-pyridine-2-sulfonyl)-hexahydro-pyrrolo[3,2-b]pyrrole-1-yl]-ethyl}-amide;
N-{1-cyclopropylmethyl-2-oxo-2-[6-oxo-4-(1-oxy-pyridine-2-sulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(1-oxy-pyridine-2-sulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-benzamide;
5-Methoxy-benzofuran-2-carboxylic acid {2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(1-oxy-1 pyridine-2-sulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(1-oxy-pyridine-2-sulfonyl)-hexahydro-pyrrolo[3,2-b]pyrrole-1-yl]-1-thiophen-2-ylmethyl-ethyl}-amide;
N-{2-oxo-2-[6-oxo-4-(1-oxy-pyridine-2-sulfonyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(1-oxy-pyridine-2-sulfonyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(1-oxy-pyridine-3-sulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
3-Methyl-benzofuran-2-carboxylic acid {1-[6-oxo-4-(1-oxy-pyridine-3-sulfonyl)-hexahydro-pyrrolo[3,2-b]pyrrole-1-carbonyl]-cyclohexyl}-amide;
N-{1-[6-oxo-4-(1-oxy-pyridine-3-sulfonyl)-hexahydro-pyrrolo[3,2-b]pyrrole-1-carbonyl]-cyclohexyl}-4-phenoxy-benzamide;
N-{1-[6-oxo-4-(1-oxy-pyridine-3-sulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(1-oxy-pyridine-3-sulfonyl)-hexahydro-pyrrolo[3,2-b]pyrrole-1-yl]-ethyl}-amide;
N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(1-oxy-pyridine-3-sulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-benzamide;
5-Methoxy-benzofuran-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(1-oxy-pyridine-3-sulfonyl)-hexahydro-pyrrolo[3,2-b]pyrrole-1-yl]-1-thiophen-2-ylmethyl-ethyl}-amide;
N-{2-oxo-2-[6-oxo-4-(1-oxy-pyridine-3-sulfonyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(1-oxy-pyridine-3-sulfonyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(pyridine-2-carbonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(pyridine-2-carbonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(pyridine-2-carbonyl)-hexahydro-pyrrolo[3,2-b]pyrrole-1-carbonyl]-cyclohexyl}-amide;
3-Methyl-benzofuran-2-carboxylic acid {1-[6-oxo-4-(pyridine-2-carbonyl)-hexahydro-pyrrolo[3,2-b]pyrrole-1-carbonyl]-cyclohexyl}-amide;
N-{1-[6-oxo-4-(pyridine-2-carbonyl)-hexahydro-pyrrolo[3,2-b]pyrrole-1-carbonyl]-cyclohexyl}-4-phenoxy-benzamide;
N-{1-[6-oxo-4-(pyridine-2-carbonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(pyridine-2-carbonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(pyridine-2-carbonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(pyridine-2-carbonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(pyridine-2-carbonyl)-hexahydro-pyrrolo[3,2-b]pyrrole-1-yl]-ethyl}-amide;
N-{1-cyclopropylmethyl-2-oxo-2-[6-oxo-4-(pyridine-2-carbonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(pyridine-2-carbonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(pyridine-2-carbonyl)-hexahydro-pyrrolo[3,2-b]pyrrol-1-yl]-1-thiophen-2-ylmethyl-ethyl}-benzamide;
5-Methoxy-benzofuran-2-carboxylic acid {2-oxo-2-[6-oxo-4-(pyridine-2-carbonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(pyridine-2-carbonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(pyridine-2-carbonyl)-hexahydro-pyrrolo[3,2-b]pyrrole-1-yl]-1-thiophen-2-ylmethyl-ethyl}-amide;
N-{2-oxo-2-[6-oxo-4-(pyridine-2-carbonyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(pyridine-2-carbonyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(2-pyridin-3-yl-acetyl)-hexahydro-pyrrolo[3,2-b]pyrrole-1-carbonyl]-cyclohexyl}-benzamide;
5-Methoxy-benzofuran-2-carboxylic acid {1-[6-oxo-4-(2-pyridin-3-yl-acetyl)-hexahydro-pyrrolo[3,2-b]pyrrole-1-carbonyl]-cyclohexyl}-amide;
Thieno[3,2-b]thiophene-2-carboxylic acid {1-[6-oxo-4-(2-pyridin-3-yl-acetyl)-hexahydro-pyrrolo[3,2-b]pyrrole-1-carbonyl]-cyclohexyl}-amide;
3-Methyl-benzofuran-2-carboxylic acid {1-[6-oxo-4-(2-pyridin-3-yl-acetyl)-hexahydro-pyrrolo[3,2-b]pyrrole-1-carbonyl]-cyclohexyl}-amide;
N-{1-[6-oxo-4-(2-pyridin-3-yl-acetyl)-hexahydro-pyrrolo[3,2-b]pyrrole-1-carbonyl]-cyclohexyl}-4-phenoxy-benzamide;
N-{1-[6-oxo-4-(2-pyridin-3-yl-acetyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(2-pyridin-3-yl-acetyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(2-pyridin-3-yl-acetyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(2-pyridin-3-yl-acetyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(2-pyridin-3-yl-acetyl)-hexahydro-pyrrolo[3,2-b]pyrrole-1-yl]-ethyl}-amide;
N-{1-cyclopropylmethyl-2-oxo-2-[6-oxo-4-(2-pyridin-3-yl-acetyl)-hexahydro-pyrrolo[3,2-b]pyrrole-1-yl]-ethyl}-4-phenoxy-benzamide;
N-{1-cyclopropylmethyl-2-oxo-2-[6-oxo-4-(2-pyridin-3-yl-acetyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(2-pyridin-3-yl-acetyl)-hexahydro-pyrrolo[3,2-b]pyrrol-1-yl]-1-thiophen-2-ylmethyl-ethyl}-benzamide;
5-Methoxy-benzofuran-2-carboxylic acid {2-oxo-2-[6-oxo-4-(2-pyridin-3-yl-acetyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(2-pyridin-3-yl-acetyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(2-pyridin-3-yl-acetyl)-hexahydro-pyrrolo[3,2-b]pyrrole-1-yl]-1-thiophen-2-ylmethyl-ethyl}-amide;
N-{2-oxo-2-[6-oxo-4-(2-pyridin-3-yl-acetyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(2-pyridin-3-yl-acetyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(pyridin-2-ylmethanesulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
3-Methyl-benzofuran-2-carboxylic acid {1-[6-oxo-4-(pyridin-2-ylmethanesulfonyl)-hexahydro-pyrrolo[3,2-b]pyrrole-1-carbonyl]-cyclohexyl}-amide;
N-{1-[6-oxo-4-(pyridin-2-ylmethanesulfonyl)-hexahydro-pyrrolo[3,2-b]pyrrole-1-carbonyl]-cyclohexyl}-4-phenoxy-benzamide;
N-{1-[6-oxo-4-(pyridin-2-ylmethanesulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(pyridin-2-ylmethanesulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(pyridin-2-ylmethanesulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(pyridin-2-ylmethanesulfonyl)-hexahydro-pyrrolo[3,2-b]pyrrole-1-yl]-ethyl}-amide;
N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(pyridin-2-ylmethanesulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-benzamide;
5-Methoxy-benzofuran-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(pyridin-2-ylmethanesulfonyl)-hexahydro-pyrrolo[3,2-b]pyrrole-1-yl]-1-thiophen-2-ylmethyl-ethyl}-amide;
N-{2-oxo-2-[6-oxo-4-(pyridin-2-ylmethanesulfonyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(pyridin-2-ylmethanesulfonyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(pyridin-3-ylmethanesulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
3-Methyl-benzofuran-2-carboxylic acid {1-[6-oxo-4-(pyridin-3-ylmethanesulfonyl)-hexahydro-pyrrolo[3,2-b]pyrrole-1-carbonyl]-cyclohexyl}-amide;
N-{1-[6-oxo-4-(pyridin-3-ylmethanesulfonyl)-hexahydro-pyrrolo[3,2-b]pyrrole-1-carbonyl]-cyclohexyl}-4-phenoxy-benzamide;
N-{1-[6-oxo-4-(pyridin-3-ylmethanesulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(pyridin-3-ylmethanesulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(pyridin-3-ylmethanesulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(pyridin-3-ylmethanesulfonyl)-hexahydro-pyrrolo[3,2-b]pyrrole-1-yl]-ethyl}-amide;
N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(pyridin-3-ylmethanesulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-1-benzamide;
5-Methoxy-benzofuran-2-carboxylic acid {2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(pyridin-3-ylmethanesulfonyl)-hexahydro-pyrrolo[3,2-b]pyrrole-1-yl]-1-thiophen-2-ylmethyl-ethyl}-amide;
N-{2-oxo-2-[6-oxo-4-(pyridin-3-ylmethanesulfonyl)-hexahydro-pyrrolo[3,2-b]pyrrol-1-yl]-1-thiophen-2-ylmethyl-ethyl}-4-phenoxy-benzamide;
N-{2-oxo-2-[6-oxo-4-(pyridin-3-ylmethanesulfonyl)-hexahydro-pyrrolo[3,2-b]pyrrol-1-yl]-1-thiophen-2-ylmethyl-ethyl}-4-thiophen-2-yl-benzamide;
4-tert-Butyl-N-{1-[6-oxo-4-(1-oxy-pyridin-2-ylmethanesulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
3-Methyl-benzofuran-2-carboxylic acid {1-[6-oxo-4-(1-oxy-pyridin-2-ylmethanesulfonyl)-hexahydro-pyrrolo[3,2-b]pyrrole-1-carbonyl]-cyclohexyl}-amide;
N-{1-[6-oxo-4-(1-oxy-pyridin-2-ylmethanesulfonyl)-hexahydro-pyrrolo[3,2-b]pyrrole-1-carbonyl]-cyclohexyl}-4-phenoxy-benzamide;
N-{1-[6-oxo-4-(1-oxy-pyridin-2-ylmethanesulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
N-{1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(1-oxy-pyridin-2-ylmethanesulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-benzamide;
5-Methoxy-benzofuran-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-amide;
N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-4-phenoxy-benzamide;
N-{2-oxo-2-[6-oxo-4-(1-oxy-pyridin-2-ylmethanesulfonyl)-hexahydro-pyrrolo[3,2-b]pyrrole-1-yl]-1-thiophen-2-ylmethyl-ethyl}-4-thiophen-2-yl-benzamide;
4-tert-Butyl-N-{1-[6-oxo-4-(1-oxy-pyridin-3-ylmethanesulfonyl)-hexahydro-pyrrolo[3,2-b]pyrrole-1-carbonyl]-cyclohexyl}-benzamide;
5-Methoxy-benzofuran-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
Thieno[3,2-b]thiophene-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
3-Methyl-benzofuran-2-carboxylic acid {1-[6-oxo-4-(1-oxy-pyridin-3-ylmethanesulfonyl)-hexahydro-pyrrolo[3,2-b]pyrrole-1-carbonyl]-cyclohexyl}-amide;
N-{1-[6-oxo-4-(1-oxy-pyridin-3-ylmethanesulfonyl)-hexahydro-pyrrolo[3,2-b]pyrrole-1-carbonyl]-cyclohexyl}-4-phenoxy-benzamide;
N-{1-[6-oxo-4-(1-oxy-pyridin-3-ylmethanesulfonyl)-hexahydro-pyrrolo[3,2-b]pyrrole-1-carbonyl]-cyclohexyl}-4-thiophen-2-yl-benzamide;
4-tert-Butyl-N-{1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-benzamide;
5-Methoxy-benzofuran-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
Thieno[3,2-b]thiophene-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
3-Methyl-benzofuran-2-carboxylic acid {1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
N-{1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-4-phenoxy-benzamide;
N-{1-cyclopropylmethyl-2-oxo-2-[6-oxo-4-(1-oxy-pyridin-3-ylmethanesulfonyl)-hexahydro-pyrrolo[3,2-b]pyrrole-1-yl]-ethyl}-4-thiophen-2-yl-benzamide;
4-tert-Butyl-N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-benzamide;
5-Methoxy-benzofuran-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
Thieno[3,2-b]thiophene-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
3-Methyl-benzofuran-2-carboxylic acid {2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-amide;
N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-4-phenoxy-benzamide;
N-{2-oxo-2-[6-oxo-4-(1-oxy-pyridin-3-ylmethanesulfonyl)-hexahydro-pyrrolo[3,2-b]pyrrole-1-yl]-1-thiophen-2-ylmethyl-ethyl}-4-thiophen-2-yl-benzamide;
2-Isobutyl-4-morpholin-4-yl-1-[6-oxo-4-(pyridine-2-sulfonyl)-hexahydro-pyrrolo[3,2-b]pyrrole-1-yl]-butane-1,4-dione;
2-Isobutyl-1-[6-oxo-4-(pyridine-2-sulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(pyridine-2-sulfonyl)-hexahydro-pyrrolo[3,2-b]pyrrole-1-yl]-butane-1,4-dione;
2-Isobutyl-1-[6-oxo-4-(pyridine-2-sulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(pyridine-2-sulfonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(pyridine-2-sulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(pyridine-2-sulfonyl)-hexahydro-pyrrolo[3,2-b]pyrrole-1-yl]-butane-1,4-dione;
2-Isobutyl-4-morpholin-4-yl-1-[6-oxo-4-(pyridine-3-sulfonyl)-hexahydro-pyrrolo[3,2-b]pyrrole-1-yl]-butane-1,4-dione;
2-Isobutyl-1-[6-oxo-4-(pyridine-3-sulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(pyridine-3-sulfonyl)-hexahydro-pyrrolo[3,2-b]pyrrole-1-yl]-butane-1,4-dione;
2-Isobutyl-1-[6-oxo-4-(pyridine-3-sulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(pyridine-3-sulfonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(pyridine-3-sulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(pyridine-3-sulfonyl)-4 hexahydro-pyrrolo[3,2-b]pyrrole-1-yl]-butane-1,4-dione;
2-Isobutyl-4-morpholin-4-yl-1-[6-oxo-4-(1-oxy-pyridine-2-sulfonyl)-hexahydro-pyrrolo[3,2-b]pyrrole-1-yl]-butane-1,4-dione;
2-Isobutyl-1-[6-oxo-4-(1-oxy-pyridine-2-sulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(1-oxy-pyridine-2-sulfonyl)-hexahydro-pyrrolo[3,2-b]pyrrole-1-yl]-butane-1,4-dione;
2-Isobutyl-1-[6-oxo-4-(1-oxy-pyridine-2-sulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(1-oxy-pyridine-2-sulfonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(1-oxy-pyridine-2-sulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(1-oxy-pyridine-2-sulfonyl)-hexahydro-pyrrolo[3,2-b]pyrrole-1-yl]-butane-1,4-dione;
2-Isobutyl-4-morpholin-4-yl-1-[6-oxo-4-(1-oxy-pyridine-3-sulfonyl)-hexahydro-pyrrolo[3,2-b]pyrrole-1-yl]-butane-1,4-dione;
2-Isobutyl-1-[6-oxo-4-(1-oxy-pyridine-3-sulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(1-oxy-pyridine-3-sulfonyl)-hexahydro-pyrrolo[3,2-b]pyrrole-1-yl]-butane-1,4-dione;
2-Isobutyl-1-[6-oxo-4-(1-oxy-pyridine-3-sulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(1-oxy-pyridine-3-sulfonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(1-oxy-pyridine-3-sulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(1-oxy-pyridine-3-sulfonyl)-hexahydro-pyrrolo[3,2-b]pyrrole-1-yl]-butane-1,4-dione;
2-Isobutyl-4-morpholin-4-yl-1-[6-oxo-4-(pyridine-2-carbonyl)-hexahydro-pyrrolo[3,2-b]pyrrole-1-yl]-butane-1,4-dione;
2-Isobutyl-1-[6-oxo-4-(pyridine-2-carbonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(pyridine-2-carbonyl)-hexahydro-pyrrolo[3,2-b]pyrrole-1-yl]-butane-1,4-dione;
2-Isobutyl-1-[6-oxo-4-(pyridine-2-carbonyl)-hexahydro-pyrrolo[3,2-b]pyrrol-1-yl]-4-(4-phenyl-piperazin-1-yl)-butane-1,4-dione;
2-Isobutyl-1-[6-oxo-4-(pyridine-2-carbonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(pyridine-2-carbonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(pyridine-2-carbonyl)-hexahydro-pyrrolo[3,2-b]pyrrole-1-yl]-butane-1,4-dione;
2-Isobutyl-4-morpholin-4-yl-1-[6-oxo-4-(2-pyridin-3-yl-acetyl)-1 hexahydro-pyrrolo[3,2-b]pyrrole-1-yl]-butane-1,4-dione;
2-Isobutyl-1-[6-oxo-4-(2-pyridin-3-yl-acetyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(2-pyridin-3-yl-acetyl)-hexahydro-pyrrolo[3,2-b]pyrrole-1-yl]-butane-1,4-dione;
2-Isobutyl-1-[6-oxo-4-(2-pyridin-3-yl-acetyl)-hexahydro-pyrrolo[3,2-b]pyrrol-1-yl]-4-(4-phenyl-piperazin-1-yl)-butane-1,4-dione;
2-Isobutyl-1-[6-oxo-4-(2-pyridin-3-yl-acetyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(2-pyridin-3-yl-acetyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(2-pyridin-3-yl-acetyl)-hexahydro-pyrrolo[3,2-b]pyrrole-1-yl]-butane-1,4-dione;
2-Isobutyl-4-morpholin-4-yl-1-[6-oxo-4-(pyridin-2-ylmethanesulfonyl)-hexahydro-pyrrolo[3,2-b]pyrrole-1-yl]-butane-1,4-dione;
2-Isobutyl-1-[6-oxo-4-(pyridin-2-ylmethanesulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(pyridin-2-ylmethanesulfonyl)-hexahydro-pyrrolo[3,2-b]pyrrole-1-yl]-butane-1,4-dione;
2-Isobutyl-1-[6-oxo-4-(pyridin-2-ylmethanesulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(pyridin-2-ylmethanesulfonyl)-hexahydro-pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(pyridin-2-ylmethanesulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(pyridin-2-ylmethanesulfonyl)-hexahydro-pyrrolo[3,2-b]pyrrole-1-yl]-butane-1,4-dione;
2-Isobutyl-4-morpholin-4-yl-1-[6-oxo-4-(pyridin-3-ylmethanesulfonyl)-hexahydro-pyrrolo[3,2-b]pyrrole-1-yl]-butane-1,4-dione;
2-Isobutyl-1-[6-oxo-4-(pyridin-3-ylmethanesulfonyl)-hexahydro-pyrrolo[3,2-b]pyrrol-1-yl]-4-piperazin-1-yl-butane-1,4-dione;
2-Isobutyl-4-(4-methyl-piperazin-1-yl)-1-[6-oxo-4-(pyridin-3-ylmethanesulfonyl)-hexahydro-pyrrolo[3,2-b]pyrrole-1-yl]-butane-1,4-dione;
2-Isobutyl-1-[6-oxo-4-(pyridin-3-ylmethanesulfonyl)-hexahydro-pyrrolo[3,2-b]pyrrol-1-yl]-4-(4-phenyl-piperazin-1-yl)-butane-1,4-dione;
2-Isobutyl-1-[6-oxo-4-(pyridin-3-ylmethanesulfonyl)-hexahydro-2 pyrrolo[3,2-b]pyrrol-1-yl]-4-(3,4,4a,8a-tetrahydro-1H-isoquinolin-2-yl)-butane-1,4-dione;
2-Isobutyl-1-[6-oxo-4-(pyridin-3-ylmethanesulfonyl)-hexahydro-pyrrolo[3,2-b]pyrrol-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(pyridin-3-ylmethanesulfonyl)-hexahydro-pyrrolo[3,2-b]pyrrole-1-yl]-butane-1,4-dione;
2-Isobutyl-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione;
2-Isobutyl-1-[6-oxo-4-(1-oxy-pyridin-2-ylmethanesulfonyl)-hexahydro-pyrrolo[3,2-b]pyrrole-1-yl]-4-piperazin-1-yl-butane-1,4-dione;
2-Isobutyl-4-(4-methyl-piperazin-1-yl)-1-[6-oxo-4-(1-oxy-pyridin-2-ylmethanesulfonyl)-hexahydro-pyrrolo[3,2-b]pyrrole-1-yl]-butane-1,4-dione;
2-Isobutyl-1-[6-oxo-4-(1-oxy-pyridin-2-ylmethanesulfonyl)-hexahydro-4 pyrrolo[3,2-b]pyrrole-1-yl]-4-(4-phenyl-piperazin-1-yl)-butane-1,4-dione;
2-Isobutyl-1-[6-oxo-4-(1-oxy-pyridin-2-ylmethanesulfonyl)-hexahydro-pyrrolo[3,2-b]pyrrole-1-yl]-4-(3,4,4a,8a-tetrahydro-1H-isoquinolin-2-yl)-butane-1,4-dione;
2-Isobutyl-1-[6-oxo-4-(1-oxy-pyridin-2-ylmethanesulfonyl)-hexahydro-pyrrolo[3,2-b]pyrrole-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(1-oxy-pyridin-2-ylmethanesulfonyl)-hexahydro-pyrrolo[3,2-b]pyrrole-1-yl]-butane-1,4-dione;
2-Isobutyl-4-morpholin-4-yl-1-[6-oxo-4-(1-oxy-pyridin-3-ylmethanesulfonyl)-hexahydro-pyrrolo[3,2-b]pyrrole-1-yl]-butane-1,4-dione;
2-Isobutyl-1-[6-oxo-4-(1-oxy-pyridin-3-ylmethanesulfonyl)-hexahydro-pyrrolo[3,2-b]pyrrole-1-yl]-4-piperazin-1-yl-butane-1,4-dione;
2-Isobutyl-4-(4-methyl-piperazin-1-yl)-1-[6-oxo-4-(1-oxy-pyridin-3-ylmethanesulfonyl)-hexahydro-pyrrolo[3,2-b]pyrrole-1-yl]-butane-1,4-dione;
2-Isobutyl-1-[6-oxo-4-(1-oxy-pyridin-3-ylmethanesulfonyl)-hexahydro-pyrrolo[3,2-b]pyrrole-1-yl]-4-(4-phenyl-piperazin-1-yl)-butane-1,4-dione;
2-Isobutyl-1-[6-oxo-4-(1-oxy-pyridin-3-ylmethanesulfonyl)-hexahydro-pyrrolo[3,2-b]pyrrole-1-yl]-4-(3,4,4a,8a-tetrahydro-1H-isoquinolin-2-yl)-butane-1,4-dione;
2-Isobutyl-1-[6-oxo-4-(1-oxy-pyridin-3-ylmethanesulfonyl)-hexahydro-pyrrolo[3,2-b]pyrrole-1-yl]-4-(1,3,3a,7a-tetrahydro-isoindol-2-yl)-butane-1,4-dione;
4-(4-Benzyl-piperazin-1-yl)-2-isobutyl-1-[6-oxo-4-(1-oxy-pyridin-3-ylmethanesulfonyl)-hexahydro-pyrrolo[3,2-b]pyrrole-1-yl]-butane-1,4-dione;
4-(2-Biphenyl-3-yl-4-methyl-pentanoyl)-1-(pyridine-2-sulfonyl)-hexahydro-pyrrolo[3,2-b]pyrrol-3-one;
4-(2-Biphenyl-3-yl-4-methyl-pentanoyl)-1-(pyridine-3-sulfonyl)-hexahydro-pyrrolo[3,2-b]pyrrol-3-one;
4-(2-Biphenyl-3-yl-4-methyl-pentanoyl)-1-(1-oxy-pyridine-2-sulfonyl)-hexahydro-pyrrolo[3,2-b]pyrrol-3-one;
4-(2-Biphenyl-3-yl-4-methyl-pentanoyl)-1-(1-oxy-pyridine-3-sulfonyl)-4 hexahydro-pyrrolo[3,2-b]pyrrol-3-one;
4-(2-Biphenyl-3-yl-4-methyl-pentanoyl)-1-(pyridine-2-carbonyl)-hexahydro-pyrrolo[3,2-b]pyrrol-3-one;
4-(2-Biphenyl-3-yl-4-methyl-pentanoyl)-1-(2-pyridin-3-yl-acetyl)-hexahydro-pyrrolo[3,2-b]pyrrol-3-one;
4-(2-Biphenyl-3-yl-4-methyl-pentanoyl)-1-(pyridin-2-ylmethanesulfonyl)-hexahydro-pyrrolo[3,2-b]pyrrol-3-one;
4-(2-Biphenyl-3-yl-4-methyl-pentanoyl)-1-(pyridin-3-ylmethanesulfonyl)-hexahydro-pyrrolo[3,2-b]pyrrol-3-one;
4-(2-Biphenyl-3-yl-4-methyl-pentanoyl)-1-(1-oxy-pyridin-2-ylmethanesulfonyl)-hexahydro-pyrrolo[3,2-b]pyrrol-3-one;
4-(2-Biphenyl-3-yl-4-methyl-pentanoyl)-1-(1-oxy-pyridin-3-1 ylmethanesulfonyl)-hexahydro-pyrrolo[3,2-b]pyrrol-3-one;
4-[4-Methyl-2-(3-pyridin-2-yl-phenyl)-pentanoyl]-1-(pyridine-2-sulfonyl)-hexahydro-pyrrolo[3,2-b]pyrrol-3-one;
4-[4-Methyl-2-(3-pyridin-2-yl-phenyl)-pentanoyl]-1-(pyridine-3-sulfonyl)-hexahydro-pyrrolo[3,2-b]pyrrol-3-one;
4-[4-Methyl-2-(3-pyridin-2-yl-phenyl)-pentanoyl]-1-(1-oxy-pyridine-2-sulfonyl)-hexahydro-pyrrolo[3,2-b]pyrrol-3-one;
4-[4-Methyl-2-(3-pyridin-2-yl-phenyl)-pentanoyl]-1-(1-oxy-pyridine-3-sulfonyl)-hexahydro-pyrrolo[3,2-b]pyrrol-3-one;
4-[4-Methyl-2-(3-pyridin-2-yl-phenyl)-pentanoyl]-1-(pyridine-2-carbonyl)-hexahydro-pyrrolo[3,2-b]pyrrol-3-one;
4-[4-Methyl-2-(3-pyridin-2-yl-phenyl)-pentanoyl]-1-(2-pyridin-3-yl-acetyl)-hexahydro-pyrrolo[3,2-b]pyrrol-3-one;
4-[4-Methyl-2-(3-pyridin-2-yl-phenyl)-pentanoyl]-1-(pyridin-2-ylmethanesulfonyl)-hexahydro-pyrrolo[3,2-b]pyrrol-3-one;
4-[4-Methyl-2-(3-pyridin-2-yl-phenyl)-pentanoyl]-1-(pyridin-3-ylmethanesulfonyl)-hexahydro-pyrrolo[3,2-b]pyrrol-3-one;
4-[4-Methyl-2-(3-pyridin-2-yl-phenyl)-pentanoyl]-1-(1-oxy-pyridin-2-ylmethanesulfonyl)-hexahydro-pyrrolo[3,2-b]pyrrol-3-one;
4-[4-Methyl-2-(3-pyridin-2-yl-phenyl)-pentanoyl]-1-(1-oxy-pyridin-3-4 ylmethanesulfonyl)-hexahydro-pyrrolo[3,2-b]pyrrol-3-one;
4-[4-Methyl-2-(3-pyridin-3-yl-phenyl)-pentanoyl]-1-(pyridine-2-sulfonyl)-hexahydro-pyrrolo[3,2-b]pyrrol-3-one;
4-[4-Methyl-2-(3-pyridin-3-yl-phenyl)-pentanoyl]-1-(pyridine-3-sulfonyl)-hexahydro-pyrrolo[3,2-b]pyrrol-3-one;
4-[4-Methyl-2-(3-pyridin-3-yl-phenyl)-pentanoyl]-1-(1-oxy-pyridine-2-sulfonyl)-hexahydro-pyrrolo[3,2-b]pyrrol-3-one;
4-[4-Methyl-2-(3-pyridin-3-yl-phenyl)-pentanoyl]-1-(1-oxy-pyridine-3-sulfonyl)-hexahydro-pyrrolo[3,2-b]pyrrol-3-one;
4-[4-Methyl-2-(3-pyridin-3-yl-phenyl)-pentanoyl]-1-(pyridine-2-carbonyl)-hexahydro-pyrrolo[3,2-b]pyrrol-3-one;
4-[4-Methyl-2-(3-pyridin-3-yl-phenyl)-pentanoyl]-1-(2-pyridin-3-yl-acetyl)-hexahydro-pyrrolo[3,2-b]pyrrol-3-one;
4-[4-Methyl-2-(3-pyridin-3-yl-phenyl)-pentanoyl]-1-(pyridin-2-ylmethanesulfonyl)-hexahydro-pyrrolo[3,2-b]pyrrol-3-one;
4-[4-Methyl-2-(3-pyridin-3-yl-phenyl)-pentanoyl]-1-(pyridin-3-ylmethanesulfonyl)-hexahydro-pyrrolo[3,2-b]pyrrol-3-one;
4-[4-Methyl-2-(3-pyridin-3-yl-phenyl)-pentanoyl]-1-(1-oxy-pyridin-2-ylmethanesulfonyl)-hexahydro-pyrrolo[3,2-b]pyrrol-3-one;
4-[4-Methyl-2-(3-pyridin-3-yl-phenyl)-pentanoyl]-1-(1-oxy-pyridin-3-ylmethanesulfonyl)-hexahydro-pyrrolo[3,2-b]pyrrol-3-one;
4-[4-Methyl-2-(3-pyridin-4-yl-phenyl)-pentanoyl]-1-(pyridine-2-sulfonyl)-hexahydro-pyrrolo[3,2-b]pyrrol-3-one;
4-[4-Methyl-2-(3-pyridin-4-yl-phenyl)-pentanoyl]-1-(pyridine-3-sulfonyl)-hexahydro-pyrrolo[3,2-b]pyrrol-3-one;
4-[4-Methyl-2-(3-pyridin-4-yl-phenyl)-pentanoyl]-1-(1-oxy-pyridine-2-sulfonyl)-hexahydro-pyrrolo[3,2-b]pyrrol-3-one;
4-[4-Methyl-2-(3-pyridin-4-yl-phenyl)-pentanoyl]-1-(1-oxy-pyridine-3-sulfonyl)-hexahydro-pyrrolo[3,2-b]pyrrol-3-one;
4-[4-Methyl-2-(3-pyridin-4-yl-phenyl)-pentanoyl]-1-(pyridine-2-carbonyl)-hexahydro-pyrrolo[3,2-b]pyrrol-3-one;
4-[4-Methyl-2-(3-pyridin-4-yl-phenyl)-pentanoyl]-1-(2-pyridin-3-yl-acetyl)-hexahydro-pyrrolo[3,2-b]pyrrol-3-one;
4-[4-Methyl-2-(3-pyridin-4-yl-phenyl)-pentanoyl]-1-(pyridin-2-ylmethanesulfonyl)-hexahydro-pyrrolo[3,2-b]pyrrol-3-one;
4-[4-Methyl-2-(3-pyridin-4-yl-phenyl)-pentanoyl]-1-(pyridin-3-ylmethanesulfonyl)-hexahydro-pyrrolo[3,2-b]pyrrol-3-one;
4-[4-Methyl-2-(3-pyridin-4-yl-phenyl)-pentanoyl]-1-(1-oxy-pyridin-2-ylmethanesulfonyl)-hexahydro-pyrrolo[3,2-b]pyrrol-3-one;
4-[4-Methyl-2-(3-pyridin-4-yl-phenyl)-pentanoyl]-1-(1-oxy-pyridin-3-ylmethanesulfonyl)-hexahydro-pyrrolo[3,2-b]pyrrol-3-one;
Furan-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
Thiophene-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(pyridine-2-sulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(pyridine-2-sulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(pyridine-2-sulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(pyridine-2-sulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(pyridine-2-sulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(pyridine-2-sulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(pyridine-2-sulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(pyridine-2-sulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(pyridine-2-sulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(pyridine-2-sulfonyl)-hexahydro-pyrrolo[3,2-b]pyrrole-1-yl]-ethyl}-amide;
Furan-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
Thiophene-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(pyridine-3-sulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(pyridine-3-sulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(pyridine-3-sulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(pyridine-3-sulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(pyridine-3-sulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(pyridine-3-sulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(pyridine-3-sulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(pyridine-3-sulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(pyridine-3-sulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(pyridine-3-sulfonyl)-hexahydro-pyrrolo[3,2-b]pyrrole-1-yl]-ethyl}-amide;
Furan-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
Thiophene-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(1-oxy-pyridine-2-sulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(1-oxy-pyridine-2-sulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(1-oxy-pyridine-2-sulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(1-oxy-pyridine-2-sulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(1-oxy-pyridine-2-sulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(1-oxy-pyridine-2-sulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(1-oxy-pyridine-2-sulfonyl)-hexahydro-pyrrolo[3,2-b]pyrrole-1-yl]-ethyl}-amide;
Furan-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
Thiophene-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(1-oxy-pyridine-3-sulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(1-oxy-pyridine-3-sulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(1-oxy-pyridine-3-sulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(1-oxy-pyridine-3-sulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(1-oxy-pyridine-3-sulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(1-oxy-pyridine-3-sulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(1-oxy-pyridine-3-sulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(1-oxy-pyridine-3-sulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(1-oxy-pyridine-3-sulfonyl)-hexahydro-pyrrolo[3,2-b]pyrrol-1-yl]-ethyl}-amide;
Biphenyl-4-carboxylic acid {1-(4-fluoro-benzyl)-2-oxo-2-[6-oxo-4-(1-oxy-pyridine-3-sulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(1-oxy-pyridine-3-sulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(1-oxy-pyridine-3-sulfonyl)-hexahydro-pyrrolo[3,2-b]pyrrole-1-yl]-ethyl}-amide;
Furan-3-carboxylic acid {3,3-dimethyl-1-[6-oxo-4-(pyridine-2-carbonyl)-hexahydro-pyrrolo[3,2-b]pyrrole-1-carbonyl]-butyl}-amide;
Thiophene-3-carboxylic acid {3,3-dimethyl-1-[6-oxo-4-(pyridine-2-carbonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(pyridine-2-carbonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(pyridine-2-carbonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(pyridine-2-carbonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(pyridine-2-carbonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(pyridine-2-carbonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(pyridine-2-carbonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(pyridine-2-carbonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(pyridine-2-carbonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(pyridine-2-carbonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(pyridine-2-carbonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(pyridine-2-carbonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(pyridine-2-carbonyl)-hexahydro-pyrrolo[3,2-b]pyrrole-1-yl]-ethyl}-amide;
Furan-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
Thiophene-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(2-pyridin-3-yl-acetyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(2-pyridin-3-yl-acetyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(2-pyridin-3-yl-acetyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(2-pyridin-3-yl-acetyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(2-pyridin-3-yl-acetyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(2-pyridin-3-yl-acetyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(2-pyridin-3-yl-acetyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(2-pyridin-3-yl-acetyl)-hexahydro-pyrrolo[3,2-b]pyrrole-1-yl]-ethyl}-amide;
Furan-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
Thiophene-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(pyridin-2-ylmethanesulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(pyridin-2-ylmethanesulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(pyridin-2-ylmethanesulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(pyridin-2-ylmethanesulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(pyridin-2-ylmethanesulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(pyridin-2-ylmethanesulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(pyridin-2-ylmethanesulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(pyridin-2-ylmethanesulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(pyridin-2-ylmethanesulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(pyridin-2-ylmethanesulfonyl)-hexahydro-pyrrolo[3,2-b]pyrrole-1-yl]-ethyl}-amide;
Furan-3-carboxylic acid {3,3-dimethyl-1-[6-oxo-4-(pyridin-3-ylmethanesulfonyl)-hexahydro-pyrrolo[3,2-b]pyrrole-1-carbonyl]-butyl}-amide;
Thiophene-3-carboxylic acid {3,3-dimethyl-1-[6-oxo-4-(pyridin-3-ylmethanesulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(pyridin-3-ylmethanesulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(pyridin-3-ylmethanesulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(pyridin-3-ylmethanesulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(pyridin-3-ylmethanesulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(pyridin-3-ylmethanesulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(pyridin-3-ylmethanesulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(pyridin-3-ylmethanesulfonyl)-hexahydro-pyrrolo[3,2-b]pyrrole-1-yl]-ethyl}-amide;
Furan-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
Thiophene-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(1-oxy-pyridin-2-ylmethanesulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(1-oxy-pyridin-2-ylmethanesulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
4-tert-Butyl-N-(1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(1-oxy-pyridin-2-ylmethanesulfonyl)-hexahydro-pyrrolo[3,2-b]pyrrole-1-yl]-ethyl}amide;
Biphenyl-4-carboxylic acid {1-(4-methoxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(1-oxy-pyridin-2-ylmethanesulfonyl)-hexahydro-pyrrolo[3,2-b]pyrrole-1-yl]-ethyl}-amide;
Furan-3-carboxylic acid {3,3-dimethyl-1-[6-oxo-4-(1-oxy-pyridin-3-1 ylmethanesulfonyl)-hexahydro-pyrrolo[3,2-b]pyrrole-1-carbonyl]-butyl}-amide;
Thiophene-3-carboxylic acid {3,3-dimethyl-1-[6-oxo-4-(1-oxy-pyridin-3-ylmethanesulfonyl)-hexahydro-pyrrolo[3,2-b]pyrrole-1-carbonyl]-butyl}-amide;
Benzo[b]thiophene-3-carboxylic acid {3,3-dimethyl-1-[6-oxo-4-(1-oxy-pyridin-3-ylmethanesulfonyl)-hexahydro-pyrrolo[3,2-b]pyrrole-1-carbonyl]-butyl}-amide;
Furan-3-carboxylic acid {1-cyclohexylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
Thiophene-3-carboxylic acid {1-cyclohexylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
Benzo[b]thiophene-3-carboxylic acid {1-cyclohexylmethyl-2-oxo-2-[6-oxo-4-(1-oxy-pyridin-3-ylmethanesulfonyl)-hexahydro-pyrrolo[3,2-b]pyrrole-1-yl]-ethyl}-amide;
Naphthalene-1-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
Quinoline-8-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
4-tert-Butyl-N-{1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
4-tert-Butyl-N-{1-(4-fluoro-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
4-tert-Butyl-N-{1-(4-methoxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
4-tert-Butyl-N-{1-(3-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-benzamide;
Biphenyl-4-carboxylic acid {1-(4-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
Biphenyl-4-carboxylic acid {1-(4-fluoro-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
Biphenyl-4-carboxylic acid {1-(4-methoxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
Biphenyl-4-carboxylic acid {1-(3-hydroxy-benzyl)-2-oxo-2-[6-oxo-4-(1-oxy-pyridin-3-ylmethanesulfonyl)-hexahydro-pyrrolo[3,2-b]pyrrole-1-yl]-ethyl}-amide;
2-Cyclohexylmethyl-4-morpholin-4-yl-1-[6-oxo-4-(pyridine-2-sulfonyl)-hexahydro-pyrrolo[3,2-b]pyrrole-1-yl]-butane-1,4-dione;
2-Cyclohexylmethyl-4-morpholin-4-yl-1-[6-oxo-4-(pyridine-3-sulfonyl)-hexahydro-pyrrolo[3,2-b]pyrrole-1-yl]-butane-1,4-dione;
2-Cyclohexylmethyl-4-morpholin-4-yl-1-[6-oxo-4-(1-oxy-pyridine-2-sulfonyl)-hexahydro-pyrrolo[3,2-b]pyrrole-1-yl]-butane-1,4-dione;
2-Cyclohexylmethyl-4-morpholin-4-yl-1-[6-oxo-4-(1-oxy-pyridine-3-sulfonyl)-hexahydro-pyrrolo[3,2-b]pyrrole-1-yl]-butane-1,4-dione;
2-Cyclohexylmethyl-4-morpholin-4-yl-1-[6-oxo-4-(pyridine-2-carbonyl)-hexahydro-pyrrolo[3,2-b]pyrrole-1-yl]-butane-1,4-dione;
2-Cyclohexylmethyl-4-morpholin-4-yl-1-[6-oxo-4-(2-pyridin-3-yl-acetyl)-hexahydro-pyrrolo[3,2-b]pyrrole-1-yl]-butane-1,4-dione;
2-Cyclohexylmethyl-4-morpholin-4-yl-1-[6-oxo-4-(pyridin-2-ylmethanesulfonyl)-hexahydro-pyrrolo[3,2-b]pyrrole-1-yl]-butane-1,4-dione;
2-Cyclohexylmethyl-4-morpholin-4-yl-1-[6-oxo-4-(pyridin-3-ylmethanesulfonyl)-hexahydro-pyrrolo[3,2-b]pyrrole-1-yl]-butane-1,4-dione;
2-Cyclohexylmethyl-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione;
2-Cyclohexylmethyl-4-morpholin-4-yl-1-[6-oxo-4-(1-oxy-pyridin-3-ylmethanesulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(pyridine-2-sulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(pyridine-3-sulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(1-oxy-pyridine-2-sulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(1-oxy-pyridine-3-sulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(pyridine-2-carbonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(2-pyridin-3-yl-acetyl)-hexahydro-pyrrolo[3,2-b]pyrrole-1-yl]-butane-1,4-dione;
2-(2,2-Dimethyl-propyl)-4-morpholin-4-yl-1-[6-oxo-4-(pyridin-2-ylmethanesulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(pyridin-3-ylmethanesulfonyl)-hexahydro-pyrrolo[3,2-b]pyrrole-1-yl]-butane-1,4-dione;
2-(2,2-Dimethyl-propyl)-4-morpholin-4-yl-1-[6-oxo-4-(1-oxy-pyridin-2-ylmethanesulfonyl)-hexahydro-pyrrolo[3,2-b]pyrrole-1-yl]-butane-1,4-dione; and
2-(2,2-Dimethyl-propyl)-4-morpholin-4-yl-1-[6-oxo-4-(1-oxy-pyridin-3-ylmethanesulfonyl)-hexahydro-pyrrolo[3,2-b]pyrrole-1-yl]-butane-1,4-dione.

51. A compound selected from the grout consisting of:
N-[1-(4-Benzoyl-6-oxo-hexahydro-pyrrolo[3,2-b]pyrrole-1-carbonyl)-3-methyl-butyl]-4-tert-butylbenzamide;
4-tert-Butyl-N-{3-methyl-1-[6-oxo-4-(1-oxy-pyridine-2-carbonyl)-hexahydro-pyrrolo[3,2-b]pyrrole-1-carbonyl]-butyl}-benzamide;
4-tert-Butyl-N-{3-methyl-1-[6-oxo-4-(pyridine-3-carbonyl)-hexahydro-pyrrolo[3,2-b]pyrrole-1-carbonyl]-butyl}-benzamide;
4-tert-Butyl-N-{3-methyl-1-[6-oxo-4-(1-oxy-pyridine-3-carbonyl)-hexahydro-pyrrolo[3,2-b]pyrrole-1-carbonyl]-butyl}-benzamide;
4-tert-Butyl-N-{3-methyl-1-[6-oxo-4-(pyridine-4-carbonyl)-hexahydro-pyrrolo[3,2-b]pyrrole-1-carbonyl]-butyl}-benzamide;
4-tert-Butyl-N-{3-methyl-1-[6-oxo-4-(1-oxy-pyridine-4-carbonyl)-hexahydro-pyrrolo[3,2-b]pyrrole-1-carbonyl]-butyl}-benzamide;
N-[1-(4-Benzenesulfonyl-6-oxo-hexahydro-pyrrolo[3,2-b]pyrrole-1-carbonyl)-3-methyl-butyl]-4-tert-butyl-benzamide;
4-tert-Butyl-N-{3-methyl-1-[6-oxo-4-(pyridine-4-sulfonyl)-hexahydro-pyrrolo[3,2-b]pyrrole-1-carbonyl]-butyl}-benzamide;
4-tert-Butyl-N-{3-methyl-1-[6-oxo-4-(1-oxy-pyridine-4-sulfonyl)-hexahydro-pyrrolo[3,2-b]pyrrole-1-carbonyl]-butyl}-benzamide;
4-tert-Butyl-N-[3-methyl-1-(6-oxo-4-phenylacetyl-hexahydro-pyrrolo[3,2-b]pyrrole-1-carbonyl)-butyl]-benzamide;
4-[2-(4-tert-Butyl-benzoylamino)-4-methyl-pentanoyl]-3-oxo-hexahydro-pyrrolo[3,2-b]pyrrole-1-carboxylic acid phenylamide;
4-[2-(4-tert-Butyl-benzoylamino)-4-methyl-pentanoyl]-3-oxo-hexahydro-pyrrolo[3,2-b]pyrrole-1-carboxylic acid isobutyl-amide;
4-[2-(4-tert-Butyl-benzoylamino)-4-methyl-pentanoyl]-3-oxo-hexahydro-pyrrolo[3,2-b]pyrrole-1-carboxylic acid phenyl ester;
4-[2-(4-tert-Butyl-benzoylamino)-4-methyl-pentanoyl]-3-oxo-hexahydro-pyrrolo[3,2-b]pyrrole-1-carboxylic acid isobutyl ester;
4-tert-Butyl-N-{3-methyl-1-[6-oxo-4-(pyrrolidine-1-carbonyl)-hexahydro-pyrrolo[3,2-b]pyrrole-1-carbonyl]-butyl}-benzamide;
4-tert-Butyl-N-{3-methyl-1-[6-oxo-4-(piperidine-1-carbonyl)-hexahydro-pyrrolo[3,2-b]pyrrole-1-carbonyl]-butyl}-benzamide;
4-tert-Butyl-N-{3-methyl-1-[4-(morpholin-4-carbonyl)-6-oxo-hexahydro-pyrrolo[3,2-b]pyrrole-1-carbonyl]-butyl}-benzamide;
4-tert-Butyl-N-{3-methyl-1-[6-oxo-4-(piperazine-1-carbonyl)-hexahydro-pyrrolo[3,2-b]pyrrole-1-carbonyl]-butyl}-benzamide;
4-tert-Butyl-N-{3-methyl-1-[4-(4-methyl-piperazine-1-carbonyl)-6-oxo-hexahydro-pyrrolo[3,2-b]pyrrole-1-carbonyl]-butyl}-benzamide;
4-tert-Butyl-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
4-tert-Butyl-N-{3-methyl-1-[6-oxo-4-(2-piperazin-1-yl-acetyl)-hexahydro-pyrrolo[3,2-b]pyrrole-1-carbonyl]-butyl}-benzamide;
4-tert-Butyl-N-{1-[4-(furan-2-carbonyl)-6-oxo-hexahydro-pyrrolo[3,2-b]pyrrole-1-carbonyl]-3-methyl-butyl}-benzamide;
4-tert-Butyl-N-{1-[4-(5-chloro-furan-2-carbonyl)-6-oxo-hexahydro-pyrrolo[3,2-b]pyrrole-1-carbonyl]-3-methyl-butyl}-benzamide;
4-tert-Butyl-N-{1-[4-(thiophene-2-carbonyl)-6-oxo-hexahydro-pyrrolo[3,2-b]pyrrole-1-carbonyl]-3-methyl-butyl}-benzamide;
4-tert-Butyl-N-{1-[4-(5-chloro-thiophene-2-carbonyl)-6-oxo-hexahydro-pyrrolo[3,2-b]pyrrole-1-carbonyl]-3-methyl-butyl}-benzamide;
4-tert-Butyl-N-{1-[4-(furan-3-carbonyl)-6-oxo-hexahydro-pyrrolo[3,2-b]pyrrole-1-carbonyl]-3-methyl-butyl}-benzamide;
4-tert-Butyl-N-{1-[4-(thiophene-3-carbonyl)-6-oxo-hexahydro-pyrrolo[3,2-b]pyrrole-1-carbonyl]-3-methyl-butyl}-benzamide;
4-tert-Butyl-N-[1-(4-cyclopentanecarbonyl-6-oxo-hexahydro-pyrrolo[3,2-b]pyrrole-1-carbonyl)-3-methyl-butyl]-benzamide;
4-tert-Butyl-N-[1-(4-cyclohexanecarbonyl-6-oxo-hexahydro-pyrrolo[3,2-b]pyrrole-1-carbonyl)-3-methyl-butyl]-benzamide;
4-tert-Butyl-N-{1-[4-(cyclopent-3-enecarbonyl)-6-oxo-hexahydro-pyrrolo[3,2-b]pyrrole-1-carbonyl]-3-methyl-butyl}-benzamide;
4-tert-Butyl-N-{1-[4-(2-cyclopentyl-acetyl)-6-oxo-hexahydro-pyrrolo[3,2-b]pyrrole-1-carbonyl]-3-methyl-butyl}-benzamide;
4-tert-Butyl-N-{1-[4-(2-cyclohexyl-acetyl)-6-oxo-hexahydro-pyrrolo[3,2-b]pyrrole-1-carbonyl]-3-methyl-butyl}-benzamide;
4-tert-Butyl-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-tert-butyl-benzamide;
4-tert-Butyl-N-{3-methyl-1-[6-oxo-4-(piperidine-2-carbonyl)-hexahydro-pyrrolo[3,2-b]pyrrole-1-carbonyl]-butyl}-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-tert-butyl-benzamide;
4-tert-Butyl-N-{3-methyl-1-[6-oxo-4-(tetrahydro-furan-2-carbonyl)-hexahydro-pyrrolo[3,2-b]pyrrole-1-carbonyl]-butyl}-benzamide;
N-{1-[4-(1-Amino-cyclobutanecarbonyl)-6-oxo-hexahydro-pyrrolo[3,2-b]pyrrole-1-carbonyl]-3-methyl-butyl}-4-tert-butyl-benzamide;
N-{1-[4-(1-Amino-cyclopentanecarbonyl)-6-oxo-hexahydro-pyrrolo[3,2-b]pyrrole-1-carbonyl]-3-methyl-butyl}-4-tert-butyl-benzamide;
N-{1-[4-(1-Amino-cyclohexanecarbonyl)-6-oxo-hexahydro-pyrrolo[3,2-b]pyrrole-1-carbonyl]-3-methyl-butyl}-4-tert-butyl-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-tert-butyl-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-tert-butyl-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-tert-butyl-benzamide;
4-tert-Butyl-N-{1-[4-(1-hydroxy-cyclobutanecarbonyl)-6-oxo-hexahydro-pyrrolo[3,2-b]pyrrole-1-carbonyl]-3-methyl-butyl}-benzamide;
4-tert-Butyl-N-{1-[4-(1-hydroxy-cyclopentanecarbonyl)-6-oxo-hexahydro-pyrrolo[3,2-b]pyrrole-1-carbonyl]-3-methyl-butyl}-benzamide;
4-tert-Butyl-N-{1-[4-(1-hydroxy-cyclohexanecarbonyl)-6-oxo-hexahydro-pyrrolo[3,2-b]pyrrole-1-carbonyl]-3-methyl-butyl}-benzamide;
4-tert-Butyl-N-{1-[4-(1-methoxy-cyclobutanecarbonyl)-6-oxo-hexahydro-pyrrolo[3,2-b]pyrrole-1-carbonyl]-3-methyl-butyl}-benzamide;
4-tert-Butyl-N-{1-[4-(1-methoxy-cyclopentanecarbonyl)-6-oxo-hexahydro-pyrrolo[3,2-b]pyrrole-1-carbonyl]-3-methyl-butyl}-benzamide;
4-tert-Butyl-N-{1-[4-(1-methoxy-cyclohexanecarbonyl)-6-oxo-hexahydro-pyrrolo[3,2-b]pyrrole-1-carbonyl]-3-methyl-butyl}-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-tert-butyl-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-tert-butyl-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-tert-butyl-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-tert-butyl-benzamide;
N-{1-[4-(2-Acetylamino-propionyl)-6-oxo-hexahydro-pyrrolo[3,2-b]pyrrole-1-carbonyl]-3-methyl-butyl}-4-tert-butyl-benzamide;
N-{1-[4-(2-Acetylamino-acetyl)-6-oxo-hexahydro-pyrrolo[3,2-b]pyrrole-1-carbonyl]-3-methyl-butyl}-4-tert-butyl-benzamide;
N-(1-{4-[2-(Acetyl-methyl-amino)-acetyl]-6-oxo-hexahydro-pyrrolo[3,2-b]pyrrole-1-carbonyl}-3-methyl-butyl)-4-tert-butyl-benzamide;
4-tert-Butyl-N-{1-[4-(2-dimethylamino-acetyl)-6-oxo-hexahydro-pyrrolo[3,2-b]pyrrole-1-carbonyl]-3-methyl-butyl}-benzamide;
N-{1-[4-(2-Amino-butyryl)-6-oxo-hexahydro-pyrrolo[3,2-b]pyrrole-1-carbonyl]-3-methyl-butyl}-4-tert-butyl-benzamide;
N-{1-[4-(2-Acetylamino-butyryl)-6-oxo-hexahydro-pyrrolo[3,2-b]pyrrole-1-carbonyl]-3-methyl-butyl}-4-tert-butyl-benzamide;
N-{1-[4-(2-amino-3-methyl-butyryl)-6-oxo-hexahydro-pyrrolo[3,2-b]pyrrole-1-carbonyl]-3-methyl-butyl}-4-tert-butyl-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-tert-butyl-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-tert-butyl-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-tert-butyl-benzamide;
N-{1-[4-(2-Amino-3-methyl-pentanoyl)-6-oxo-hexahydro-pyrrolo[3,2-b]pyrrole-1-carbonyl]-3-methyl-butyl}-4-tert-butyl-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-tert-butyl-benzamide;
4-tert-Butyl-N-[3-methyl-1-(6-oxo-4-pentanoyl-hexahydro-pyrrolo[3,2-b]pyrrole-1-carbonyl)-butyl]-benzamide;
4-tert-Butyl-N-{3-methyl-1-[4-(3-methyl-pentanoyl)-6-oxo-hexahydro-pyrrolo[3,2-b]pyrrole-1-carbonyl]-butyl}-benzamide;
4-tert-Butyl-N-{3-methyl-1-[4-(4-methyl-pentanoyl)-6-oxo-hexahydro-pyrrolo[3,2-b]pyrrole-1-carbonyl]-butyl}-benzamide;
N-{1-[4-(2-Amino-4-methyl-pentanoyl)-6-oxo-hexahydro-pyrrolo[3,2-b]pyrrole-1-carbonyl]-3-methyl-butyl}-4-tert-butyl-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-tert-butyl-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-tert-butyl-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-tert-butyl-benzamide;
N-{1-[4-(2-Amino-pentanoyl)-6-oxo-hexahydro-pyrrolo[3,2-b]pyrrole-1-carbonyl]-3-methyl-butyl}-4-tert-butyl-benzamide;
N-{1-[4-(2-Acetylamino-pentanoyl)-6-oxo-hexahydro-pyrrolo[3,2-b]pyrrole-1-carbonyl]-3-methyl-butyl}-4-tert-butyl-benzamide;
N-{1-[4-(2-Amino-5-methyl-hexanoyl)-6-oxo-hexahydro-pyrrolo[3,2-b]pyrrole-1-carbonyl]-3-methyl-butyl}-4-tert-butyl-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-tert-butyl-benzamide;
N-{1-[4-(2-hydroxy-3-methyl-butyryl)-6-oxo-hexahydro-pyrrolo[3,2-b]pyrrole-1-carbonyl]-3-methyl-butyl}-4-tert-butyl-benzamide;
N-{1-[4-(2-methoxy-3-methyl-butyryl)-6-oxo-hexahydro-pyrrolo[3,2-b]pyrrole-1-carbonyl]-3-methyl-butyl}-4-tert-butyl-benzamide;
4-tert-Butyl-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
4-tert-Butyl-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
4-tert-Butyl-N-{1-[4-(2-hydroxy-3-methyl-pentanoyl)-6-oxo-hexahydro-pyrrolo[3,2-b]pyrrole-1-carbonyl]-3-methyl-butyl}-benzamide;
4-tert-Butyl-N-{1-[4-(2-methoxy-3-methyl-pentanoyl)-6-oxo-hexahydro-pyrrolo[3,2-b]pyrrole-1-carbonyl]-3-methyl-butyl}-benzamide;
4-tert-Butyl-N-{1-[4-(2-hydroxy-4-methyl-pentanoyl)-6-oxo-hexahydro-pyrrolo[3,2-b]pyrrole-1-carbonyl]-3-methyl-butyl}-benzamide;
4-tert-Butyl-N-{1-[4-(2-methoxy-4-methyl-pentanoyl)-6-oxo-hexahydro-pyrrolo[3,2-b]pyrrole-1-carbonyl]-3-methyl-butyl}-benzamide;
4-tert-Butyl-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
4-tert-Butyl-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
4-tert-Butyl-N-{1-[4-(2-hydroxy-pentanoyl)-6-oxo-hexahydro-pyrrolo[3,2-b]pyrrole-1-carbonyl]-3-methyl-butyl}-benzamide;
4-tert-Butyl-N-{1-[4-(2-methoxy-pentanoyl)-6-oxo-hexahydro-pyrrolo[3,2-b]pyrrole-1-carbonyl]-3-methyl-butyl}-benzamide;
4-tert-Butyl-N-{1-[4-(2-hydroxy-5-methyl-hexanoyl)-6-oxo-hexahydro-pyrrolo[3,2-b]pyrrole-1-carbonyl]-3-methyl-butyl}-benzamide;
4-tert-Butyl-N-{1-[4-(2-methoxy-5-methyl-hexanoyl)-6-oxo-hexahydro-pyrrolo[3,2-b]pyrrole-1-carbonyl]-3-methyl-butyl}-benzamide;
N-[1-(4-Benzoyl-6-oxo-hexahydro-pyrrolo[3,2-b]pyrrole-1-carbonyl)-3-methyl-butyl]-4-dimethylaminobenzamide;
4-Dimethylamino-N-{3-methyl-1-[6-oxo-4-(1-oxy-pyridine-2-carbonyl)-hexahydro-pyrrolo[3,2-b]pyrrole-1-carbonyl]-butyl}-benzamide;
4-Dimethylamino-N-{3-methyl-1-[6-oxo-4-(pyridine-3-carbonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridine-3-carbonyl)-hexahydro-pyrrolo[3,2-b]pyrrole-1-carbonyl]-butyl}-benzamide;
4-Dimethylamino-N-{3-methyl-1-[6-oxo-4-(pyridine-4-carbonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridine-4-carbonyl)-hexahydro-pyrrolo[3,2-b]pyrrole-1-carbonyl]-butyl}-benzamide;
N-[1-(4-Benzenesulfonyl-6-oxo-hexahydro-pyrrolo[3,2-b]pyrrole-1-carbonyl)-3-methyl-butyl]-4-dimethylamino-benzamide;
4-Dimethylamino-N-{3-methyl-1-[6-oxo-4-(pyridine-4-sulfonyl)-hexahydro-pyrrolo[3,2-b]pyrrole-1-carbonyl]-butyl}-benzamide;
4-Dimethylamino-N-{3-methyl-1-[6-oxo-4-(1-oxy-pyridine-4-sulfonyl)-hexahydro-pyrrolo[3,2-b]pyrrole-1-carbonyl]-butyl}-benzamide;
4-Dimethylamino-N-[3-methyl-1-(6-oxo-4-phenylacetyl-hexahydro-pyrrolo[3,2-b]pyrrole-1-carbonyl)-butyl]-benzamide;
4-[2-(4-Dimethylamino-benzoylamino)-4-methyl-pentanoyl]-3-oxo-hexahydro-pyrrolo[3,2-b]pyrrole-1-carboxylic acid phenylamide;
4-[2-(4-Dimethylamino-benzoylamino)-4-methyl-pentanoyl]-3-oxo-hexahydro-pyrrolo[3,2-b]pyrrole-1-carboxylic acid isobutyl-amide;
4-[2-(4-Dimethylamino-benzoylamino)-4-methyl-pentanoyl]-3-oxo-hexahydro-pyrrolo[3,2-b]pyrrole-1-carboxylic acid phenyl ester;
4-[2-(4-Dimethylamino-benzoylamino)-4-methyl-pentanoyl]-3-oxo-hexahydro-pyrrolo[3,2-b]pyrrole-1-carboxylic acid isobutyl ester;
4-Dimethylamino-N-{3-methyl-1-[6-oxo-4-(pyrrolidine-1-carbonyl)-hexahydro-pyrrolo[3,2-b]pyrrole-1-carbonyl]-butyl}-benzamide;
4-Dimethylamino-N-{3-methyl-1-[6-oxo-4-(piperidine-1-carbonyl)-hexahydro-pyrrolo[3,2-b]pyrrole-1-carbonyl]-butyl}-benzamide;
4-Dimethylamino-N-{3-methyl-1-[4-(morpholin-4-carbonyl)-6-oxo-hexahydro-pyrrolo[3,2-b]pyrrole-1-carbonyl]-butyl}-benzamide;
4-Dimethylamino-N-{3-methyl-1-[6-oxo-4-(piperazine-1-carbonyl)-hexahydro-pyrrolo[3,2-b]pyrrole-1-carbonyl]-butyl}-benzamide;
4-Dimethylamino-N-{3-methyl-1-[4-(4-methyl-piperazine-1-carbonyl)-6-oxo-hexahydro-pyrrolo[3,2-b]pyrrole-1-carbonyl]-butyl}-benzamide;
4-Dimethylamino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
4-Dimethylamino-N-{3-methyl-1-[6-oxo-4-(2-piperazin-1-yl-acetyl)-hexahydro-pyrrolo[3,2-b]pyrrole-1-carbonyl]-butyl}-benzamide;
4-Dimethylamino-N-{1-[4-(furan-2-carbonyl)-6-oxo-hexahydro-pyrrolo[3,2-b]pyrrole-1-carbonyl]-3-methyl-butyl}-benzamide;
4-Dimethylamino-N-{1-[4-(5-chloro-furan-2-carbonyl)-6-oxo-hexahydro-pyrrolo[3,2-b]pyrrole-1-carbonyl]-3-methyl-butyl}-benzamide;
4-Dimethylamino-N-{1-[4-(thiophene-2-carbonyl)-6-oxo-hexahydro-pyrrolo[3,2-b]pyrrole-1-carbonyl]-3-methyl-butyl}-benzamide;
4-Dimethylamino-N-{1-[4-(5-chloro-thiophene-2-carbonyl)-6-oxo-hexahydro-pyrrolo[3,2-b]pyrrole-1-carbonyl]-3-methyl-butyl}-benzamide;
4-Dimethylamino-N-{1-[4-(furan-3-carbonyl)-6-oxo-hexahydro-pyrrolo[3,2-b]pyrrole-1-carbonyl]-3-methyl-butyl}-benzamide;
4-Dimethylamino-N-{1-[4-(thiophene-3-carbonyl)-6-oxo-hexahydro-pyrrolo[3,2-b]pyrrole-1-carbonyl]-3-methyl-butyl}-benzamide;
4-Dimethylamino-N-[1-(4-cyclopentanecarbonyl-6-oxo-hexahydro-pyrrolo[3,2-b]pyrrole-1-carbonyl)-3-methyl-butyl]-benzamide;
4-Dimethylamino-N-[1-(4-cyclohexanecarbonyl-6-oxo-hexahydro-pyrrolo[3,2-b]pyrrole-1-carbonyl)-3-methyl-butyl]-benzamide;
4-Dimethylamino-N-{1-[4-(cyclopent-3-enecarbonyl)-6-oxo-hexahydro-pyrrolo[3,2-b]pyrrole-1-carbonyl]-3-methyl-butyl}-benzamide;
4-Dimethylamino-N-{1-[4-(2-cyclopentyl-acetyl)-6-oxo-hexahydro-pyrrolo[3,2-b]pyrrole-1-carbonyl]-3-methyl-butyl}-benzamide;
4-Dimethylamino-N-{1-[4-(2-cyclohexyl-acetyl)-6-oxo-hexahydro-pyrrolo[3,2-b]pyrrole-1-carbonyl]-3-methyl-butyl}-benzamide;
4-Dimethylamino-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-dimethylamino-benzamide;
4-Dimethylamino-N-{3-methyl-1-[6-oxo-4-(piperidine-2-carbonyl)-hexahydro-pyrrolo[3,2-b]pyrrole-1-carbonyl]-butyl}-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-dimethylamino-benzamide;
4-Dimethylamino-N-{3-methyl-1-[6-oxo-4-(tetrahydro-furan-2-carbonyl)-hexahydro-pyrrolo[3,2-b]pyrrole-1-carbonyl]-butyl}-benzamide;
N-{1-[4-(1-Amino-cyclobutanecarbonyl)-6-oxo-hexahydro-pyrrolo[3,2-b]pyrrole-1-carbonyl]-3-methyl-butyl}-4-dimethylamino-benzamide;
N-{1-[4-(1-Amino-cyclopentanecarbonyl)-6-oxo-hexahydro-pyrrolo[3,2-b]pyrrole-1-carbonyl]-3-methyl-butyl}-4-dimethylamino-benzamide;
N-{1-[4-(1-Amino-cyclohexanecarbonyl)-6-oxo-hexahydro-pyrrolo[3,2-b]pyrrole-1-carbonyl]-3-methyl-butyl}-4-dimethylamino-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-dimethylamino-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-dimethylamino-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-dimethylamino-benzamide;
4-Dimethylamino-N-{1-[4-(1-hydroxy-cyclobutanecarbonyl)-6-oxo-hexahydro-pyrrolo[3,2-b]pyrrole-1-carbonyl]-3-methyl-butyl}-benzamide;
4-Dimethylamino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
4-Dimethylamino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
4-Dimethylamino-N-{1-[4-(1-methoxy-cyclobutanecarbonyl)-6-oxo-hexahydro-pyrrolo[3,2-b]pyrrole-1-carbonyl]-3-methyl-butyl}-benzamide;
4-Dimethylamino-N-{1-[4-(1-methoxy-cyclopentanecarbonyl)-6-oxo-hexahydro-pyrrolo[3,2-b]pyrrole-1-carbonyl]-3-methyl-butyl}-benzamide;
4-Dimethylamino-N-{1-[4-(1-methoxy-cyclohexanecarbonyl)-6-oxo-hexahydro-pyrrolo[3,2-b]pyrrole-1-carbonyl]-3-methyl-butyl}-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-dimethylamino-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-dimethylamino-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-dimethylamino-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-dimethylamino-benzamide;
N-{1-[4-(2-Acetylamino-propionyl)-6-oxo-hexahydro-pyrrolo[3,2-b]pyrrole-1-carbonyl]-3-methyl-butyl}-4-dimethylamino-benzamide;
N-{1-[4-(2-Acetylamino-acetyl)-6-oxo-hexahydro-pyrrolo[3,2-b]pyrrole-1-carbonyl]-3-methyl-butyl}-4-dimethylamino-benzamide;
N-(1-{4-[2-(Acetyl-methyl-amino)-acetyl]-6-oxo-hexahydro-pyrrolo[3,2-b]pyrrole-1-carbonyl}-3-methyl-butyl)-4-dimethylamino-benzamide;
4-Dimethylamino-N-{1-[4-(2-dimethylamino-acetyl)-6-oxo-hexahydro-pyrrolo[3,2-b]pyrrole-1-carbonyl]-3-methyl-butyl}-benzamide;
N-{1-[4-(2-Amino-butyryl)-6-oxo-hexahydro-pyrrolo[3,2-b]pyrrole-1-carbonyl]-3-methyl-butyl}-4-dimethylamino-benzamide;
N-{1-[4-(2-Acetylamino-butyryl)-6-oxo-hexahydro-pyrrolo[3,2-b]pyrrole-1-carbonyl]-3-methyl-butyl}-4-dimethylamino-benzamide;
N-{1-[4-(2-amino-3-methyl-butyryl)-6-oxo-hexahydro-pyrrolo[3,2-b]pyrrole-1-carbonyl]-3-methyl-butyl}-4-dimethylamino-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-dimethylamino-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-dimethylamino-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-dimethylamino-benzamide;
N-{1-[4-(2-Amino-3-methyl-pentanoyl)-6-oxo-hexahydro-pyrrolo[3,2-b]pyrrole-1-carbonyl]-3-methyl-butyl}-4-dimethylamino-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-dimethylamino-benzamide;
4-Dimethylamino-N-[3-methyl-1-(6-oxo-4-pentanoyl-hexahydro-pyrrolo[3,2-b]pyrrole-1-carbonyl)-butyl]-benzamide;
4-Dimethylamino-N-{3-methyl-1-[4-(3-methyl-pentanoyl)-6-oxo-hexahydro-pyrrolo[3,2-b]pyrrole-1-carbonyl]-butyl}-benzamide;
4-Dimethylamino-N-{3-methyl-1-[4-(4-methyl-pentanoyl)-6-oxo-hexahydro-pyrrolo[3,2-b]pyrrole-1-carbonyl]-butyl}-benzamide;
N-{1-[4-(2-Amino-4-methyl-pentanoyl)-6-oxo-hexahydro-pyrrolo[3,2-b]pyrrole-1-carbonyl]-3-methyl-butyl}-4-dimethylamino-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-dimethylamino-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-dimethylamino-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-dimethylamino-benzamide;
N-{1-[4-(2-Amino-pentanoyl)-6-oxo-hexahydro-pyrrolo[3,2-b]pyrrole-1-carbonyl]-3-methyl-butyl}-4-dimethylamino-benzamide;
N-{1-[4-(2-Acetylamino-pentanoyl)-6-oxo-hexahydro-pyrrolo[3,2-b]pyrrole-1-carbonyl]-3-methyl-butyl}-4-dimethylamino-benzamide;
N-{1-[4-(2-Amino-5-methyl-hexanoyl)-6-oxo-hexahydro-pyrrolo[3,2-b]pyrrole-1-carbonyl]-3-methyl-butyl}-4-dimethylamino-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-dimethylamino-benzamide;
N-{1-[4-(2-hydroxy-3-methyl-butyryl)-6-oxo-hexahydro-pyrrolo[3,2-b]pyrrole-1-carbonyl]-3-methyl-butyl}-4-dimethylamino-benzamide;
N-{1-[4-(2-methoxy-3-methyl-butyryl)-6-oxo-hexahydro-pyrrolo[3,2-b]pyrrole-1-carbonyl]-3-methyl-butyl}-4-dimethylamino-benzamide;
4-Dimethylamino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
4-Dimethylamino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
4-Dimethylamino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
4-Dimethylamino-N-{1-[4-(2-methoxy-3-methyl-pentanoyl)-6-oxo-hexahydro-pyrrolo[3,2-b]pyrrole-1-carbonyl]-3-methyl-butyl}-benzamide;
4-Dimethylamino-N-{1-[4-(2-hydroxy-4-methyl-pentanoyl)-6-oxo-hexahydro-pyrrolo[3,2-b]pyrrole-1-carbonyl]-3-methyl-butyl}-benzamide;
4-Dimethylamino-N-{1-[4-(2-methoxy-4-methyl-pentanoyl)-6-oxo-hexahydro-pyrrolo[3,2-b]pyrrole-1-carbonyl]-3-methyl-butyl}-benzamide;
4-Dimethylamino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
4-Dimethylamino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
4-Dimethylamino-N-{1-[4-(2-hydroxy-pentanoyl)-6-oxo-hexahydro-pyrrolo[3,2-b]pyrrole-1-carbonyl]-3-methyl-butyl}-benzamide;
4-Dimethylamino-N-{1-[4-(2-methoxy-pentanoyl)-6-oxo-hexahydro-pyrrolo[3,2-b]pyrrole-1-carbonyl]-3-methyl-butyl}-benzamide;
4-Dimethylamino-N-{1-[4-(2-hydroxy-5-methyl-hexanoyl)-6-oxo-hexahydro-pyrrolo[3,2-b]pyrrole-1-carbonyl]-3-methyl-butyl}-benzamide;
4-Dimethylamino-N-{1-[4-(2-methoxy-5-methyl-hexanoyl)-6-oxo-hexahydro-pyrrolo[3,2-b]pyrrole-1-carbonyl]-3-methyl-butyl}-benzamide;
N-[1-(4-Benzoyl-6-oxo-hexahydro-pyrrolo[3,2-b]pyrrole-1-carbonyl)-3-methyl-butyl]-4-thiophen-2-yl-benzamide;
N-{3-methyl-1-[6-oxo-4-(1-oxy-pyridine-2-carbonyl)-hexahydro-pyrrolo[3,2-b]pyrrole-1-carbonyl]-butyl}-4-thiophen-2-yl-benzamide;
N-{3-methyl-1-[6-oxo-4-(pyridine-3-carbonyl)-hexahydro-pyrrolo[3,2-b]pyrrole-1-carbonyl]-butyl}-4-thiophen-2-yl-benzamide;
N-{3-methyl-1-[6-oxo-4-(1-oxy-pyridine-3-carbonyl)-hexahydro-pyrrolo[3,2-b]pyrrole-1-carbonyl]-butyl}-4-thiophen-2-yl-benzamide;
N-{3-methyl-1-[6-oxo-4-(pyridine-4-carbonyl)-hexahydro-pyrrolo[3,2-b]pyrrole-1-carbonyl]-butyl}-4-thiophen-2-yl-benzamide;
N-{3-methyl-1-[6-oxo-4-(1-oxy-pyridine-4-carbonyl)-hexahydro-pyrrolo[3,2-b]pyrrole-1-carbonyl]-butyl}-4-thiophen-2-yl-benzamide;
N-[1-(4-Benzenesulfonyl-6-oxo-hexahydro-pyrrolo[3,2-b]pyrrole-1-carbonyl)-3-methyl-butyl]-4-thiophen-2-yl-benzamide;
N-{3-methyl-1-[6-oxo-4-(pyridine-4-sulfonyl)-hexahydro-pyrrolo[3,2-b]pyrrole-1-carbonyl]-butyl}-4-thiophen-2-yl-benzamide;
N-{3-methyl-1-[6-oxo-4-(1-oxy-pyridine-4-sulfonyl)-hexahydro-pyrrolo[3,2-b]pyrrole-1-carbonyl]-butyl}-4-thiophen-2-yl-benzamide;
N-[3-methyl-1-(6-oxo-4-phenylacetyl-hexahydro-pyrrolo[3,2-b]pyrrole-1-carbonyl)-butyl]-4-thiophen-2-yl-benzamide;
4-[4-Methyl-2-(4-thiophen-2-yl-benzoylamino)-pentanoyl]-3-oxo-hexahydro-pyrrolo[3,2-b]pyrrole-1-carboxylic acid phenylamide;
4-[4-Methyl-2-(4-thiophen-2-yl-benzoylamino)-pentanoyl]-3-oxo-hexahydro-pyrrolo[3,2-b]pyrrole-1-carboxylic acid isobutyl-amide;
4-[4-Methyl-2-(4-thiophen-2-yl-benzoylamino)-pentanoyl]-3-oxo-hexahydro-pyrrolo[3,2-b]pyrrole-1-carboxylic acid phenyl ester;
4-[4-Methyl-2-(4-thiophen-2-yl-benzoylamino)-pentanoyl]-3-oxo-hexahydro-pyrrolo[3,2-b]pyrrole-1-carboxylic acid isobutyl ester;
N-{3-methyl-1-[6-oxo-4-(pyrrolidine-1-carbonyl)-hexahydro-pyrrolo[3,2-b]pyrrole-1-carbonyl]-butyl}-4-thiophen-2-yl-benzamide;
N-{3-methyl-1-[6-oxo-4-(piperidine-1-carbonyl)-hexahydro-pyrrolo[3,2-b]pyrrole-1-carbonyl]-butyl}-4-thiophen-2-yl-benzamide;
N-{3-methyl-1-[4-(morpholin-4-carbonyl)-6-oxo-hexahydro-pyrrolo[3,2-b]pyrrole-1-carbonyl]-butyl}-4-thiophen-2-yl-benzamide;
N-{3-methyl-1-[6-oxo-4-(piperazine-1-carbonyl)-hexahydro-pyrrolo[3,2-b]pyrrole-1-carbonyl]-butyl}-4-thiophen-2-yl-benzamide;
N-{3-methyl-1-[4-(4-methyl-piperazine-1-carbonyl)-6-oxo-hexahydro-pyrrolo[3,2-b]pyrrole-1-carbonyl]-butyl}-4-thiophen-2-yl-benzamide;
N-(3-methyl-1-{4-[2-(4-methyl-piperazin-1-yl)-acetyl]-6-oxo-hexahydro-pyrrolo[3,2-b]pyrrole-1-carbonyl}-butyl)-4-thiophen-2-yl-benzamide;
N-{3-methyl-1-[6-oxo-4-(2-piperazin-1-yl-acetyl)-hexahydro-pyrrolo[3,2-b]pyrrole-1-carbonyl]-butyl}-4-thiophen-2-yl-benzamide;
N-{1-[4-(furan-2-carbonyl)-6-oxo-hexahydro-pyrrolo[3,2-b]pyrrole-1-carbonyl]-3-methyl-butyl}-4-thiophen-2-yl-benzamide;
N-{1-[4-(5-chloro-furan-2-carbonyl)-6-oxo-hexahydro-pyrrolo[3,2-b]pyrrole-1-carbonyl]-3-methyl-butyl}-4-thiophen-2-yl-benzamide;
N-{1-[4-(thiophene-2-carbonyl)-6-oxo-hexahydro-pyrrolo[3,2-b]pyrrole-1-carbonyl]-3-methyl-butyl}-4-thiophen-2-yl-benzamide;
N-{1-[4-(5-chloro-thiophene-2-carbonyl)-6-oxo-hexahydro-pyrrolo[3,2-b]pyrrole-1-carbonyl]-3-methyl-butyl}-4-thiophen-2-yl-benzamide;
N-{1-[4-(furan-3-carbonyl)-6-oxo-hexahydro-pyrrolo[3,2-b]pyrrole-1-carbonyl]-3-methyl-butyl}-4-thiophen-2-yl-benzamide;
N-{1-[4-(thiophene-3-carbonyl)-6-oxo-hexahydro-pyrrolo[3,2-b]pyrrole-1-carbonyl]-3-methyl-butyl}-4-thiophen-2-yl-benzamide;
N-[1-(4-cyclopentanecarbonyl-6-oxo-hexahydro-pyrrolo[3,2-b]pyrrole-1-carbonyl)-3-methyl-butyl]-4-thiophen-2-yl-benzamide;
N-[1-(4-cyclohexanecarbonyl-6-oxo-hexahydro-pyrrolo[3,2-b]pyrrole-1-carbonyl)-3-methyl-butyl]-4-thiophen-2-yl-benzamide;
N-{1-[4-(cyclopent-3-enecarbonyl)-6-oxo-hexahydro-pyrrolo[3,2-b]pyrrole-1-carbonyl]-3-methyl-butyl}-4-thiophen-2-yl-benzamide;
N-{1-[4-(2-cyclopentyl-acetyl)-6-oxo-hexahydro-pyrrolo[3,2-b]pyrrole-1-carbonyl]-3-methyl-butyl}-4-thiophen-2-yl-benzamide;
N-{1-[4-(2-cyclohexyl-acetyl)-6-oxo-hexahydro-pyrrolo[3,2-b]pyrrole-1-carbonyl]-3-methyl-butyl}-4-thiophen-2-yl-benzamide;
N-{3-methyl-1-[6-oxo-4-(pyrrolidine-2-carbonyl)-hexahydro-pyrrolo[3,2-b]pyrrole-1-carbonyl]-butyl}-4-thiophen-2-yl-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-thiophen-2-yl-benzamide;
N-{3-methyl-1-[6-oxo-4-(piperidine-2-carbonyl)-hexahydro-pyrrolo[3,2-b]pyrrole-1-carbonyl]-butyl}-4-thiophen-2-yl-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-thiophen-2-yl-benzamide;
N-{3-methyl-1-[6-oxo-4-(tetrahydro-furan-2-carbonyl)-hexahydro-pyrrolo[3,2-b]pyrrole-1-carbonyl]-butyl}-4-thiophen-2-yl-benzamide;
N-{1-[4-(1-Amino-cyclobutanecarbonyl)-6-oxo-hexahydro-pyrrolo[3,2-b]pyrrole-1-carbonyl]-3-methyl-butyl}-4-thiophen-2-yl-benzamide;
N-{1-[4-(1-Amino-cyclopentanecarbonyl)-6-oxo-hexahydro-pyrrolo[3,2-b]pyrrole-1-carbonyl]-3-methyl-butyl}-4-thiophen-2-yl-benzamide;
N-{1-[4-(1-Amino-cyclohexanecarbonyl)-6-oxo-hexahydro-pyrrolo[3,2-b]pyrrole-1-carbonyl]-3-methyl-butyl}-4-thiophen-2-yl-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-thiophen-2-yl-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-thiophen-2-yl-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-thiophen-2-yl-benzamide;
N-{1-[4-(1-hydroxy-cyclobutanecarbonyl)-6-oxo-hexahydro-pyrrolo[3,2-b]pyrrole-1-carbonyl]-3-methyl-butyl}-4-thiophen-2-yl-benzamide;
N-{1-[4-(1-hydroxy-cyclopentanecarbonyl)-6-oxo-hexahydro-pyrrolo[3,2-b]pyrrole-1-carbonyl]-3-methyl-butyl}-4-thiophen-2-yl-benzamide;
N-{1-[4-(1-hydroxy-cyclohexanecarbonyl)-6-oxo-hexahydro-pyrrolo[3,2-b]pyrrole-1-carbonyl]-3-methyl-butyl}-4-thiophen-2-yl-benzamide;
N-{1-[4-(1-methoxy-cyclobutanecarbonyl)-6-oxo-hexahydro-pyrrolo[3,2-b]pyrrole-1-carbonyl]-3-methyl-butyl}-4-thiophen-2-yl-benzamide;
N-{1-[4-(1-methoxy-cyclopentanecarbonyl)-6-oxo-hexahydro-pyrrolo[3,2-b]pyrrole-1-carbonyl]-3-methyl-butyl}-4-thiophen-2-yl-benzamide;
N-{1-[4-(1-methoxy-cyclohexanecarbonyl)-6-oxo-hexahydro-pyrrolo[3,2-b]pyrrole-1-carbonyl]-3-methyl-butyl}-4-thiophen-2-yl-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-thiophen-2-yl-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-propionyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-acetyl)-6-oxo-hexahydro-pyrrolo[3,2-b]pyrrole-1-carbonyl]-3-methyl-butyl}-4-thiophen-2-yl-benzamide;
N-(1-{4-[2-(Acetyl-methyl-amino)-acetyl]-6-oxo-hexahydro-pyrrolo[3,2-b]pyrrole-1-carbonyl}-3-methyl-butyl]-4-thiophen-2-yl-benzamide;
N-{1-[4-(2-dimethylamino-acetyl)-6-oxo-hexahydro-pyrrolo[3,2-b]pyrrole-1-carbonyl]-3-methyl-butyl}-4-thiophen-2-yl-benzamide;
N-{1-[4-(2-Amino-butyryl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-butyryl)-6-oxo-hexahydro-pyrrolo[3,2-b]pyrrole-1-carbonyl]-3-methyl-butyl}-4-thiophen-2-yl-benzamide;
N-{1-[4-(2-amino-3-methyl-butyryl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-thiophen-2-yl-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-thiophen-2-yl-benzamide;
N-{1-[4-(2-Amino-3-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-thiophen-2-yl-benzamide;
N-[3-methyl-1-(6-oxo-4-pentanoyl-hexahydro-pyrrolo[3,2-b]pyrrole-1-carbonyl)-butyl]-4-thiophen-2-yl-benzamide;
N-{3-methyl-1-[4-(3-methyl-pentanoyl)-6-oxo-hexahydro-pyrrolo[3,2-b]pyrrole-1-carbonyl]-butyl}-4-thiophen-2-yl-benzamide;
N-{3-methyl-1-[4-(4-methyl-pentanoyl)-6-oxo-hexahydro-pyrrolo[3,2-b]pyrrole-1-carbonyl]-butyl}-4-thiophen-2-yl-benzamide;
N-{1-[4-(2-Amino-4-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Amino-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-pentanoyl)-6-oxo-hexahydro-pyrrolo[3,2-b]pyrrole-1-carbonyl]-3-methyl-butyl}-4-thiophen-2-yl-benzamide;
N-{1-[4-(2-Amino-5-methyl-hexanoyl)-6-oxo-hexahydro-pyrrolo[3,2-b]pyrrole-1-carbonyl]-3-methyl-butyl}-4-thiophen-2-yl-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-thiophen-2-yl-benzamide;
N-{1-[4-(2-hydroxy-3-methyl-butyryl)-6-oxo-hexahydro-pyrrolo[3,2-b]pyrrole-1-carbonyl]-3-methyl-butyl}-4-thiophen-2-yl-benzamide;
N-{1-[4-(2-methoxy-3-methyl-butyryl)-6-oxo-hexahydro-pyrrolo[3,2-b]pyrrole-1-carbonyl]-3-methyl-butyl}-4-thiophen-2-yl-benzamide;
N-{1-[4-(2-hydroxy-3,3-dimethyl-butyryl)-6-oxo-hexahydro-pyrrolo[3,2-b]pyrrole-1-carbonyl]-3-methyl-butyl}-4-thiophen-2-yl-benzamide;
N-{1-[4-(2-methoxy-3,3-dimethyl-butyryl)-6-oxo-hexahydro-pyrrolo[3,2-b]pyrrole-1-carbonyl]-3-methyl-butyl}-4-thiophen-2-yl-benzamide;
N-{1-[4-(2-hydroxy-3-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-methoxy-3-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-hydroxy-4-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-methoxy-4-methyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-hydroxy-4,4-dimethyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-methoxy-4,4-dimethyl-pentanoyl)-6-oxo-hexahydro-pyrrolo[3,2-b]pyrrole-1-carbonyl]-3-methyl-butyl}-4-thiophen-2-yl-benzamide;
N-{1-[4-(2-hydroxy-pentanoyl)-6-oxo-hexahydro-pyrrolo[3,2-b]pyrrole-1-carbonyl]-3-methyl-butyl}-4-thiophen-2-yl-benzamide;
N-{1-[4-(2-methoxy-pentanoyl)-6-oxo-hexahydro-pyrrolo[3,2-b]pyrrole-1-carbonyl]-3-methyl-butyl}-4-thiophen-2-yl-benzamide;
N-{1-[4-(2-hydroxy-5-methyl-hexanoyl)-6-oxo-hexahydro-pyrrolo[3,2-b]pyrrole-1-carbonyl]-3-methyl-butyl}-4-thiophen-2-yl-benzamide;
N-{1-[4-(2-methoxy-5-methyl-hexanoyl)-6-oxo-hexahydro-pyrrolo[3,2-b]pyrrole-1-carbonyl]-3-methyl-butyl}-4-thiophen-2-yl-benzamide;
5-Phenyl-thiophene-2-carboxylic acid-[1-(4-Benzoyl-6-oxo-hexahydro-pyrrolo[3,2-b]pyrrole-1-carbonyl)-3-methyl-butyl]-amide;
5-Phenyl-thiophene-2-carboxylic acid-{3-methyl-1-[6-oxo-4-(pyridine-2-carbonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-2-carbonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(pyridine-3-carbonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-3-carbonyl)-hexahydro-pyrrolo[3,2-b]pyrrole-1-carbonyl]-butyl}-amide;
5-Phenyl-thiophene-2-carboxylic acid-{(3-methyl-1-[6-oxo-4-(pyridine-4-carbonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-4-carbonyl)-hexahydro-pyrrolo[3,2-b]pyrrole-1-carbonyl]-butyl}-amide;
5-Phenyl-thiophene-2-carboxylic acid-[1-(4-Benzenesulfonyl-6-oxo-hexahydro-pyrrolo[3,2-b]pyrrole-1-carbonyl)-3-methyl-butyl]-amide;
5-Phenyl-thiophene-2-carboxylic acid-{3-methyl-1-[6-oxo-4-(pyridine-2-sulfonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-2-sulfonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(pyridine-3-sulfonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-3-sulfonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(pyridine-4-sulfonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(1-oxy-pyridine-4-sulfonyl)-hexahydro-pyrrolo[3,2-b]pyrrole-1-carbonyl]-butyl}-amide;
5-Phenyl-thiophene-2-carboxylic acid-[3-methyl-1-(6-oxo-4-phenylacetyl-hexahydro-pyrrolo[3,2-b]pyrrole-1-carbonyl)-butyl]-amide;
4-{4-Methyl-2-[(5-phenyl-thiophene-2-carbonyl)-amino]-pentanoyl}-3-oxo-hexahydro-pyrrolo[3,2-b]pyrrole-1-carboxylic acid phenylamide;
4-{4-Methyl-2-[(5-phenyl-thiophene-2-carbonyl)-amino]-pentanoyl}-3-oxo-hexahydro-pyrrolo[3,2-b]pyrrole-1-carboxylic acid isobutyl-amide;
4-{4-Methyl-2-[(5-phenyl-thiophene-2-carbonyl)-amino]-pentanoyl}-3-oxo-hexahydro-pyrrolo[3,2-b]pyrrole-1-carboxylic acid phenyl ester;
4-{4-Methyl-2-[(5-phenyl-thiophene-2-carbonyl)-amino]-pentanoyl}-3-oxo-hexahydro-pyrrolo[3,2-b]pyrrole-1-carboxylic acid isobutyl ester;
5-Phenyl-thiophene-2-carboxylic acid-{3-methyl-1-[6-oxo-4-(pyrrolidine-1-carbonyl)-hexahydro-pyrrolo[3,2-b]pyrrole-1-carbonyl]-butyl}-amide;
5-Phenyl-thiophene-2-carboxylic acid-{3-methyl-1-[6-oxo-4-(piperidine-1-carbonyl)-hexahydro-pyrrolo[3,2-b]pyrrole-1-carbonyl]-butyl}-amide;
5-Phenyl-thiophene-2-carboxylic acid-{3-methyl-1-[4-(morpholin-4-carbonyl)-6-oxo-hexahydro-pyrrolo[3,2-b]pyrrole-1-carbonyl]-butyl}-amide;
5-Phenyl-thiophene-2-carboxylic acid-{3-methyl-1-[6-oxo-4-(piperazine-1-carbonyl)-hexahydro-pyrrolo[3,2-b]pyrrole-1-carbonyl]-butyl}-amide;
5-Phenyl-thiophene-2-carboxylic acid-{3-methyl-1-[4-(4-methyl-piperazine-1-carbonyl)-6-oxo-hexahydro-pyrrolo[3,2-b]pyrrole-1-carbonyl]-butyl}-amide;
5-Phenyl-thiophene-2-carboxylic acid-(3-methyl-1-{4-[2-(4-methyl-piperazin-1-yl)-acetyl]-6-oxo-hexahydro-pyrrolo[3,2-b]pyrrole-1-carbonyl}-butyl)-amide;
5-Phenyl-thiophene-2-carboxylic acid-{3-methyl-1-[6-oxo-4-(2-piperazin-1-yl-acetyl)-hexahydro-pyrrolo[3,2-b]pyrrole-1-carbonyl]-butyl}-amide;
5-Phenyl-thiophene-2-carboxylic acid-{1-[4-(furan-2-carbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(5-chloro-furan-2-carbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(thiophene-2-carbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(5-chloro-thiophene-2-carbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(furan-3-carbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(thiophene-3-carbonyl)-6-oxo-hexahydro-pyrrolo[3,2-b]pyrrole-1-carbonyl]-3-methyl-butyl}-amide;
5-Phenyl-thiophene-2-carboxylic acid-[1-(4-cyclopentanecarbonyl-6-oxo-hexahydro-pyrrolo[3,2-b]pyrrole-1-carbonyl)-3-methyl-butyl]-amide;
5-Phenyl-thiophene-2-carboxylic acid-[1-(4-cyclohexanecarbonyl-6-oxo-hexahydro-pyrrolo[3,2-b]pyrrole-1-carbonyl)-3-methyl-butyl]-amide;
5-Phenyl-thiophene-2-carboxylic acid-{1-[4-(cyclopent-3-enecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-cyclopentyl-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-cyclohexyl-acetyl)-6-oxo-hexahydro-pyrrolo[3,2-b]pyrrole-1-carbonyl]-3-methyl-butyl}-amide;
5-Phenyl-thiophene-2-carboxylic acid-{3-methyl-1-[6-oxo-4-(pyrrolidine-2-carbonyl)-hexahydro-pyrrolo[3,2-b]pyrrole-1-carbonyl]-butyl}-amide;
5-Phenyl-thiophene-2-carboxylic acid-{1-[4-(1-Acetyl-pyrrolidine-2-carbonyl)-6-oxo-hexahydro-pyrrolo[3,2-b]pyrrole-1-carbonyl]-3-methyl-butyl}-amide;
5-Phenyl-thiophene-2-carboxylic acid-{3-methyl-1-[6-oxo-4-(piperidine-2-carbonyl)-hexahydro-pyrrolo[3,2-b]pyrrole-1-carbonyl]-butyl}-amide;
5-Phenyl-thiophene-2-carboxylic acid-{1-[4-(1-Acetyl-piperidine-2-carbonyl)-6-oxo-hexahydro-pyrrolo[3,2-b]pyrrole-1-carbonyl]-3-methyl-butyl}-amide;
5-Phenyl-thiophene-2-carboxylic acid-{3-methyl-1-[6-oxo-4-(tetrahydro-furan-2-carbonyl)-hexahydro-pyrrolo[3,2-b]pyrrole-1-carbonyl]-butyl}-amide;
5-Phenyl-thiophene-2-carboxylic acid-{1-[4-(1-Amino-cyclobutanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-Amino-cyclopentanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-Amino-cyclohexanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-Acetylamino-cyclobutanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-Acetylamino-cyclopentanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-Acetylamino-cyclohexanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-hydroxy-cyclobutanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-hydroxy-cyclopentanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-hydroxy-cyclohexanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-methoxy-cyclobutanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-methoxy-cyclopentanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(1-methoxy-cyclohexanecarbonyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-2-cyclopentyl-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-2-cyclohexyl-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-2-cyclopentyl-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-2-cyclohexyl-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-propionyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-acetyl)-6-oxo-hexahydro-pyrrolo[3,2-b]pyrrole-1-carbonyl]-3-methyl-butyl}-amide;
5-Phenyl-thiophene-2-carboxylic acid-(1-{4-[2-(Acetyl-methyl-amino)-acetyl]-6-oxo-hexahydro-pyrrolo[3,2-b]pyrrole-1-carbonyl}-3-methyl-butyl)-amide;
5-Phenyl-thiophene-2-carboxylic acid-{1-[4-(2-dimethylamino-acetyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-amino-3-methyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-3-methyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-amino-3,3-dimethyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-3,3-dimethyl-butyryl)-6-oxo-hexahydro-pyrrolo[3,2-b]pyrrole-1-carbonyl]-3-methyl-butyl 1-amide;
5-Phenyl-thiophene-2-carboxylic acid-{1-[4-(2-Amino-3-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-3-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-[3-methyl-1-(6-oxo-4-pentanoyl-hexahydro-pyrrolo[3,2-b]pyrrole-1-carbonyl)-butyl]-amide;
5-Phenyl-thiophene-2-carboxylic acid-{3-methyl-1-[4-(3-methyl-pentanoyl)-6-oxo-hexahydro-pyrrolo[3,2-b]pyrrole-1-carbonyl]-butyl}-amide;
5-Phenyl-thiophene-2-carboxylic acid-{3-methyl-1-[4-(4-methyl-pentanoyl)-6-oxo-hexahydro-pyrrolo[3,2-b]pyrrole-1-carbonyl]-butyl}-amide;
5-Phenyl-thiophene-2-carboxylic acid-{1-[4-(2-Amino-4-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-4-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-4,4-dimethyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-4,4-dimethyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-pentanoyl)-6-oxo-hexahydro-pyrrolo[3,2-b]pyrrole-1-carbonyl]-3-methyl-butyl}-amide;
5-Phenyl-thiophene-2-carboxylic acid {1-[4-(2-Acetylamino-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Amino-5-methyl-hexanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-Acetylamino-5-methyl-hexanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-hydroxy-3-methyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-3-methyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-hydroxy-3,3-dimethyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-3,3-dimethyl-butyryl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-hydroxy-3-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-3-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-hydroxy-4-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-4-methyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-hydroxy-4,4-dimethyl-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-4,4-dimethyl-pentanoyl)-6-oxo-hexahydro-pyrrolo[3,2-b]pyrrole-1-carbonyl]-3-methyl-butyl}-amide;
5-Phenyl-thiophene-2-carboxylic acid=-{1-[4-(2-hydroxy-pentanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-pentanoyl)-6-oxo-hexahydro-pyrrolo[3,2-b]pyrrole-1-carbonyl]-3-methyl-butyl}-amide;
5-Phenyl-thiophene-2-carboxylic acid-1-[4-(2-hydroxy-5-methyl-hexanoyl)-6-oxo-hexahydro-pyrrolo[3,2-b]pyrrole-1-carbonyl]-3-methyl-butyl}-amide;
5-Phenyl-thiophene-2-carboxylic acid-{1-[4-(2-methoxy-5-methyl-hexanoyl)-6-oxo-hexahydro-pyrrolo[3,2-b]pyrrole-1-carbonyl]-3-methyl-butyl}-amide;
N-[1-(4-Benzoyl-6-oxo-hexahydro-pyrrolo[3,2-b]pyrrole-1-carbonyl)-3-methyl-butyl]-4-pyrrolidin-1-ylbenzamide;
N-{3-methyl-1-[6-oxo-4-(pyridine-2-carbonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-2-carbonyl)-hexahydro-pyrrolo[3,2-b]pyrrole-1-carbonyl]-butyl}-4-pyrrolidin-1-yl-benzamide;
N-{3-methyl-1-[6-oxo-4-(pyridine-3-carbonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-3-carbonyl)-hexahydro-pyrrolo[3,2-b]pyrrole-1-carbonyl]-butyl}-4-pyrrolidin-1-yl-benzamide;
N-{3-methyl-1-[6-oxo-4-(pyridine-4-carbonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-4-carbonyl)-hexahydro-pyrrolo[3,2-b]pyrrole-1-carbonyl]-butyl}-4-pyrrolidin-1-yl-benzamide;
N-[1-(4-Benzenesulfonyl-6-oxo-hexahydro-pyrrolo[3,2-b]pyrrole-1-carbonyl)-3-methyl-butyl]-4-pyrrolidin-1-yl-benzamide;
N-{3-methyl-1-[6-oxo-4-(pyridine-2-sulfonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-2-sulfonyl)-hexahydro-pyrrolo[3,2-b]pyrrole-1-carbonyl]-butyl}-4-pyrrolidin-1-yl-benzamide;
N-{3-methyl-1-[6-oxo-4-(pyridine-3-sulfonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-3-sulfonyl)-hexahydro-pyrrolo[3,2-b]pyrrole-1-carbonyl]-butyl}-4-pyrrolidin-1-yl-benzamide;
N-{3-methyl-1-[6-oxo-4-(pyridine-4-sulfonyl)-hexahydro-pyrrolo[3,2-b]pyrrole-1-carbonyl]-butyl}-4-pyrrolidin-1-yl-benzamide;
N-{3-methyl-1-[6-oxo-4-(1-oxy-pyridine-4-sulfonyl)-hexahydro-pyrrolo[3,2-b]pyrrole-1-carbonyl]-butyl}-4-pyrrolidin-1-yl-benzamide;
N-[3-methyl-1-(6-oxo-4-phenylacetyl-hexahydro-pyrrolo[3,2-b]pyrrole-1-carbonyl)-butyl]-4-pyrrolidin-1-yl-benzamide;
4-[2-(4-pyrrolidin-1-yl-benzoylamino)-4-methyl-pentanoyl]-3-oxo-hexahydro-pyrrolo[3,2-b]pyrrole-1-carboxylic acid phenylamide;
4-[2-(4-pyrrolidin-1-yl-benzoylamino)-4-methyl-pentanoyl]-3-oxo-hexahydro-pyrrolo[3,2-b]pyrrole-1-carboxylic acid isobutyl-amide;
4-[2-(4-pyrrolidin-1-yl-benzoylamino)-4-methyl-pentanoyl]-3-oxo-hexahydro-pyrrolo[3,2-b]pyrrole-1-carboxylic acid phenyl ester;
4-[2-(4-pyrrolidin-1-yl-benzoylamino)-4-methyl-pentanoyl]-3-oxo-hexahydro-pyrrolo[3,2-b]pyrrole-1-carboxylic acid isobutyl ester;
N-{3-methyl-1-[6-oxo-4-(pyrrolidine-1-carbonyl)-hexahydro-pyrrolo[3,2-b]pyrrole-1-carbonyl]-butyl}-4-pyrrolidin-1-yl-benzamide;
N-{3-methyl-1-[6-oxo-4-(piperidine-1-carbonyl)-hexahydro-pyrrolo[3,2-b]pyrrole-1-carbonyl]-butyl}-4-pyrrolidin-1-yl-benzamide;
N-{3-methyl-1-[4-(morpholin-4-carbonyl)-6-oxo-hexahydro-pyrrolo[3,2-b]pyrrole-1-carbonyl]-butyl}-4-pyrrolidin-1-yl-benzamide;
N-{3-methyl-1-[6-oxo-4-(piperazine-1-carbonyl)-hexahydro-pyrrolo[3,2-b]pyrrole-1-carbonyl]-butyl}-4-pyrrolidin-1-yl-benzamide;
N-{3-methyl-1-[4-(4-methyl-piperazine-1-carbonyl)-6-oxo-hexahydro-pyrrolo[3,2-b]pyrrole-1-carbonyl]-butyl}-4-pyrrolidin-1-yl-benzamide;
N-(3-methyl-1-{4-[2-(4-methyl-piperazin-1-yl)-acetyl]-6-oxo-hexahydro-pyrrolo[3,2-b]pyrrole-1-carbonyl}-butyl)-4-pyrrolidin-1-yl-benzamide;
N-{3-methyl-1-[6-oxo-4-(2-piperazin-1-yl-acetyl)-hexahydro-pyrrolo[3,2-b]pyrrole-1-carbonyl]-butyl}-4-pyrrolidin-1-yl-benzamide;
N-{1-[4-(furan-2-carbonyl)-6-oxo-hexahydro-pyrrolo[3,2-b]pyrrole-1-carbonyl]-3-methyl-butyl}-4-pyrrolidin-1-yl-benzamide;
N-{1-[4-(5-chloro-furan-2-carbonyl)-6-oxo-hexahydro-pyrrolo[3,2-b]pyrrole-1-carbonyl]-3-methyl-butyl}-4-pyrrolidin-1-yl-benzamide;
N-{1-[4-(thiophene-2-carbonyl)-6-oxo-hexahydro-pyrrolo[3,2-b]pyrrole-1-carbonyl]-3-methyl-butyl}-4-pyrrolidin-1-yl-benzamide;
N-{1-[4-(5-chloro-thiophene-2-carbonyl)-6-oxo-hexahydro-pyrrolo[3,2-b]pyrrole-1-carbonyl]-3-methyl-butyl}-4-pyrrolidin-1-yl-benzamide;
N-{1-[4-(furan-3-carbonyl)-6-oxo-hexahydro-pyrrolo[3,2-b]pyrrole-1-carbonyl]-3-methyl-butyl}-4-pyrrolidin-1-yl-benzamide;
N-{1-[4-(thiophene-3-carbonyl)-6-oxo-hexahydro-pyrrolo[3,2-b]pyrrole-1-carbonyl]-3-methyl-butyl}-4-pyrrolidin-1-yl-benzamide;
N-[1-(4-cyclopentanecarbonyl-6-oxo-hexahydro-pyrrolo[3,2-b]pyrrole-1-carbonyl)-3-methyl-butyl]-4-pyrrolidin-1-yl-benzamide;
N-[1-(4-cyclohexanecarbonyl-6-oxo-hexahydro-pyrrolo[3,2-b]pyrrole-1-carbonyl)-3-methyl-butyl]-4-pyrrolidin-1-yl-benzamide;
N-{1-[4-(cyclopent-3-enecarbonyl)-6-oxo-hexahydro-pyrrolo[3,2-b]pyrrole-1-carbonyl]-3-methyl-butyl}-4-pyrrolidin-1-yl-benzamide;
N-{1-[4-(2-cyclopentyl-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-cyclohexyl-acetyl)-6-oxo-hexahydro-pyrrolo[3,2-b]pyrrole-1-carbonyl]-3-methyl-butyl}-4-pyrrolidin-1-yl-benzamide;
4-Pyrrolidin-1-yl-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-pyrrolidin-1-yl-benzamide;
N-{3-methyl-1-[6-oxo-4-(piperidine-2-carbonyl)-hexahydro-pyrrolo[3,2-b]pyrrole-1-carbonyl]-butyl}-4-pyrrolidin-1-yl-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-pyrrolidin-1-yl-benzamide;
N-{3-methyl-1-[6-oxo-4-(tetrahydro-furan-2-carbonyl)-hexahydro-pyrrolo[3,2-b]pyrrole-1-carbonyl]-butyl}-4-pyrrolidin-1-yl-benzamide;
N-{1-[4-(1-Amino-cyclobutanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-Amino-cyclopentanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-Amino-cyclohexanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-hydroxy-cyclobutanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-hydroxy-cyclopentanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-hydroxy-cyclohexanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-methoxy-cyclobutanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-methoxy-cyclopentanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(1-methoxy-cyclohexanecarbonyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-propionyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-acetyl)-6-oxo-hexahydro-pyrrolo[3,2-b]pyrrole-1-carbonyl]-3-methyl-butyl}-4-pyrrolidin-1-yl-benzamide;
N-(1-{4-[2-(Acetyl-methyl-amino)-acetyl]-6-oxo-hexahydro-pyrrolo[3,2-b]pyrrole-1-carbonyl}-3-methyl-butyl]-4-pyrrolidin-1-yl-benzamide;
N-{1-[4-(2-dimethylamino-acetyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-amino-3-methyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-3-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-pyrrolidin-1-yl-benzamide;
N-[3-methyl-1-(6-oxo-4-pentanoyl-hexahydro-pyrrolo[3,2-b]pyrrole-1-carbonyl)-butyl]-4-pyrrolidin-1-yl-benzamide;
N-{3-methyl-1-[4-(3-methyl-pentanoyl)-6-oxo-hexahydro-pyrrolo[3,2-b]pyrrole-1-carbonyl]-butyl}-4-pyrrolidin-1-yl-benzamide;
N-{3-methyl-1-[4-(4-methyl-pentanoyl)-6-oxo-hexahydro-pyrrolo[3,2-b]pyrrole-1-carbonyl]-butyl}-4-pyrrolidin-1-yl-benzamide;
N-{1-[4-(2-Amino-4-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Amino-5-methyl-hexanoyl)-6-oxo-hexahydro-pyrrolo[3,2-b]pyrrole-1-carbonyl]-3-methyl-butyl}-4-pyrrolidin-1-yl-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-3-methyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-3-methyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-3,3-dimethyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-3,3-dimethyl-butyryl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-3-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-3-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-4-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-4-methyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-4,4-dimethyl-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-pentanoyl)-6-oxo-hexahydro-pyrrolo[3,2-b]pyrrole-1-carbonyl]-3-methyl-butyl}-4-pyrrolidin-1-yl-benzamide;
N-{1-[4-(2-hydroxy-5-methyl-hexanoyl)-6-oxo-hexahydro-pyrrolo[3,2-b]pyrrole-1-carbonyl]-3-methyl-butyl}-4-pyrrolidin-1-yl-benzamide;
N-{1-[4-(2-methoxy-5-methyl-hexanoyl)-6-oxo-hexahydro-pyrrolo[3,2-b]pyrrole-1-carbonyl]-3-methyl-butyl}-4-pyrrolidin-1-yl-benzamide;
N-[1-(4-Benzoyl-6-oxo-hexahydro-pyrrolo[3,2-b]pyrrole-1-carbonyl)-3-methyl-butyl]-4-morpholin-4-ylbenzamide;
N-{3-methyl-1-[6-oxo-4-(1-oxy-pyridine-2-carbonyl)-hexahydro-pyrrolo[3,2-b]pyrrole-1-carbonyl]-butyl}-4-morpholin-4-yl-benzamide;
N-{3-methyl-1-[6-oxo-4-(pyridine-3-carbonyl)-hexahydro-pyrrolo[3,2-b]pyrrole-1-carbonyl]-butyl}-4-morpholin-4-yl-benzamide;
N-{3-methyl-1-[6-oxo-4-(1-oxy-pyridine-3-carbonyl)-hexahydro-pyrrolo[3,2-b]pyrrole-1-carbonyl]-butyl}-4-morpholin-4-yl-benzamide;
N-{3-methyl-1-[6-oxo-4-(pyridine-4-carbonyl)-hexahydro-pyrrolo[3,2-b]pyrrole-1-carbonyl]-butyl}-4-morpholin-4-yl-benzamide;
N-{3-methyl-1-[6-oxo-4-(1-oxy-pyridine-4-carbonyl)-hexahydro-pyrrolo[3,2-b]pyrrole-1-carbonyl]-butyl}-4-morpholin-4-yl-benzamide;
N-[1-(4-Benzenesulfonyl-6-oxo-hexahydro-pyrrolo[3,2-b]pyrrole-1-carbonyl)-3-methyl-butyl]-4-morpholin-4-yl-benzamide;
N-{3-methyl-1-[6-oxo-4-(pyridine-4-sulfonyl)-hexahydro-pyrrolo[3,2-b]pyrrole-1-carbonyl]-butyl}-4-morpholin-4-yl-benzamide;
N-{3-methyl-1-[6-oxo-4-(1-oxy-pyridine-4-sulfonyl)-hexahydro-pyrrolo[3,2-b]pyrrole-1-carbonyl]-butyl}-4-morpholin-4-yl-benzamide;
N-[3-methyl-1-(6-oxo-4-phenylacetyl-hexahydro-pyrrolo[3,2-b]pyrrole-1-carbonyl)-butyl]-4-morpholin-4-yl-benzamide;
4-[2-(4-morpholin-4-yl-benzoylamino)-4-methyl-pentanoyl]-3-oxo-hexahydro-pyrrolo[3,2-b]pyrrole-1-carboxylic acid phenylamide;
4-[2-(4-morpholin-4-yl-benzoylamino)-4-methyl-pentanoyl]-3-oxo-hexahydro-pyrrolo[3,2-b]pyrrole-1-carboxylic acid isobutyl-amide;
4-[2-(4-morpholin-4-yl-benzoylamino)-4-methyl-pentanoyl]-3-oxo-hexahydro-pyrrolo[3,2-b]pyrrole-1-carboxylic acid phenyl ester;
4-[2-(4-morpholin-4-yl-benzoylamino)-4-methyl-pentanoyl]-3-oxo-hexahydro-pyrrolo[3,2-b]pyrrole-1-carboxylic acid isobutyl ester;
N-{3-methyl-1-[6-oxo-4-(pyrrolidine-1-carbonyl)-hexahydro-pyrrolo[3,2-b]pyrrole-1-carbonyl]-butyl}-4-morpholin-4-yl-benzamide;
N-{3-methyl-1-[6-oxo-4-(piperidine-1-carbonyl)-hexahydro-pyrrolo[3,2-b]pyrrole-1-carbonyl]-butyl}-4-morpholin-4-yl-benzamide;
N-{3-methyl-1-[4-(morpholin-4-carbonyl)-6-oxo-hexahydro-pyrrolo[3,2-b]pyrrole-1-carbonyl]-butyl}-4-morpholin-4-yl-benzamide;
N-{3-methyl-1-[6-oxo-4-(piperazine-1-carbonyl)-hexahydro-pyrrolo[3,2-b]pyrrole-1-carbonyl]-butyl}-4-morpholin-4-yl-benzamide;
N-{3-methyl-1-[4-(4-methyl-piperazine-1-carbonyl)-6-oxo-hexahydro-pyrrolo[3,2-b]pyrrole-1-carbonyl]-butyl}-4-morpholin-4-yl-benzamide;
N-(3-methyl-1-{4-[2-(4-methyl-piperazin-1-yl)-acetyl]-6-oxo-hexahydro-pyrrolo[3,2-b]pyrrole-1-carbonyl}-butyl)-4-morpholin-4-yl-benzamide;
N-{3-methyl-1-[6-oxo-4-(2-piperazin-1-yl-acetyl)-hexahydro-pyrrolo[3,2-b]pyrrole-1-carbonyl]-butyl}-4-morpholin-4-yl-benzamide;
N-{1-[4-(furan-2-carbonyl)-6-oxo-hexahydro-pyrrolo[3,2-b]pyrrole-1-carbonyl]-3-methyl-butyl}-4-morpholin-4-yl-benzamide;
N-{1-[4-(5-chloro-furan-2-carbonyl)-6-oxo-hexahydro-pyrrolo[3,2-b]pyrrole-1-carbonyl]-3-methyl-butyl}-4-morpholin-4-yl-benzamide;
N-{1-[4-(thiophene-2-carbonyl)-6-oxo-hexahydro-pyrrolo[3,2-b]pyrrole-1-carbonyl]-3-methyl-butyl}-4-morpholin-4-yl-benzamide;
N-{1-[4-(5-chloro-thiophene-2-carbonyl)-6-oxo-hexahydro-pyrrolo[3,2-b]pyrrole-1-carbonyl]-3-methyl-butyl}-4-morpholin-4-yl-benzamide;
N-{1-[4-(furan-3-carbonyl)-6-oxo-hexahydro-pyrrolo[3,2-b]pyrrole-1-carbonyl]-3-methyl-butyl}-4-morpholin-4-yl-benzamide;
N-{1-[4-(thiophene-3-carbonyl)-6-oxo-hexahydro-pyrrolo[3,2-b]pyrrole-1-carbonyl]-3-methyl-butyl}-4-morpholin-4-yl-benzamide;
N-[1-(4-cyclopentanecarbonyl-6-oxo-hexahydro-pyrrolo[3,2-b]pyrrole-1-carbonyl)-3-methyl-butyl]-4-morpholin-4-yl-benzamide;
N-[1-(4-cyclohexanecarbonyl-6-oxo-hexahydro-pyrrolo[3,2-b]pyrrole-1-carbonyl)-3-methyl-butyl]-4-morpholin-4-yl-benzamide;
N-{1-[4-(cyclopent-3-enecarbonyl)-6-oxo-hexahydro-pyrrolo[3,2-b]pyrrole-1-carbonyl]-3-methyl-butyl}-4-morpholin-4-yl-benzamide;
N-{1-[4-(2-cyclopentyl-acetyl)-6-oxo-hexahydro-pyrrolo[3,2-b]pyrrole-1-carbonyl]-3-methyl-butyl}-4-morpholin-4-yl-benzamide;
N-{1-[4-(2-cyclohexyl-acetyl)-6-oxo-hexahydro-pyrrolo[3,2-b]pyrrole-1-carbonyl]-3-methyl-butyl}-4-morpholin-4-yl-benzamide;
4-morpholin-4-yl-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-morpholin-4-yl-benzamide;
N-{3-methyl-1-[6-oxo-4-(piperidine-2-carbonyl)-hexahydro-pyrrolo[3,2-b]pyrrole-1-carbonyl]-butyl}-4-morpholin-4-yl-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-morpholin-4-yl-benzamide;
N-{3-methyl-1-[6-oxo-4-(tetrahydro-furan-2-carbonyl)-hexahydro-pyrrolo[3,2-b]pyrrole-1-carbonyl]-butyl}-4-morpholin-4-yl-benzamide;
N-{1-[4-(1-Amino-cyclobutanecarbonyl)-6-oxo-hexahydro-pyrrolo[3,2-b]pyrrole-1-carbonyl]-3-methyl-butyl}-4-morpholin-4-yl-benzamide;
N-{1-[4-(1-Amino-cyclopentanecarbonyl)-6-oxo-hexahydro-pyrrolo[3,2-b]pyrrole-1-carbonyl]-3-methyl-butyl}-4-morpholin-4-yl-benzamide;
N-{1-[4-(1-Amino-cyclohexanecarbonyl)-6-oxo-hexahydro-pyrrolo[3,2-b]pyrrole-1-carbonyl]-3-methyl-butyl}-4-morpholin-4-yl-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-morpholin-4-yl-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-morpholin-4-yl-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-morpholin-4-yl-benzamide;
N-{1-[4-(1-hydroxy-cyclobutanecarbonyl)-6-oxo-hexahydro-pyrrolo[3,2-b]pyrrole-1-carbonyl]-3-methyl-butyl}-4-morpholin-4-yl-benzamide;
N-{1-[4-(1-hydroxy-cyclopentanecarbonyl)-6-oxo-hexahydro-pyrrolo[3,2-b]pyrrole-1-carbonyl]-3-methyl-butyl}-4-morpholin-4-yl-benzamide;
N-{1-[4-(1-hydroxy-cyclohexanecarbonyl)-6-oxo-hexahydro-pyrrolo[3,2-b]pyrrole-1-carbonyl]-3-methyl-butyl}-4-morpholin-4-yl-benzamide;
N-{1-[4-(1-methoxy-cyclobutanecarbonyl)-6-oxo-hexahydro-pyrrolo[3,2-b]pyrrole-1-carbonyl]-3-methyl-butyl}-4-morpholin-4-yl-benzamide;
N-{1-[4-(1-methoxy-cyclopentanecarbonyl)-6-oxo-hexahydro-pyrrolo[3,2-b]pyrrole-1-carbonyl]-3-methyl-butyl}-4-morpholin-4-yl-benzamide;
N-{1-[4-(1-methoxy-cyclohexanecarbonyl)-6-oxo-hexahydro-pyrrolo[3,2-b]pyrrole-1-carbonyl]-3-methyl-butyl}-4-morpholin-4-yl-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-morpholin-4-yl-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-propionyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-acetyl)-6-oxo-hexahydro-pyrrolo[3,2-b]pyrrole-1-carbonyl]-3-methyl-butyl}-4-morpholin-4-yl-benzamide;
N-(1-{4-[2-(Acetyl-methyl-amino)-acetyl]-6-oxo-hexahydro-pyrrolo[3,2-b]pyrrole-1-carbonyl}-3-methyl-butyl]-4-morpholin-4-yl-benzamide;
N-{1-[4-(2-dimethylamino-acetyl)-6-oxo-hexahydro-pyrrolo[3,2-b]pyrrole-1-carbonyl]-3-methyl-butyl}-4-morpholin-4-yl-benzamide;
N-{1-[4-(2-Amino-butyryl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-butyryl)-6-oxo-hexahydro-pyrrolo[3,2-b]pyrrole-1-carbonyl]-3-methyl-butyl}-4-morpholin-4-yl-benzamide;
N-{1-[4-(2-amino-3-methyl-butyryl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-morpholin-4-yl-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-morpholin-4-yl-benzamide;
N-{1-[4-(2-Amino-3-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-morpholin-4-yl-benzamide;
N-[3-methyl-1-(6-oxo-4-pentanoyl-hexahydro-pyrrolo[3,2-b]pyrrole-1-carbonyl)-butyl]-4-morpholin-4-yl-benzamide;
N-{3-methyl-1-[4-(3-methyl-pentanoyl)-6-oxo-hexahydro-pyrrolo[3,2-b]pyrrole-1-carbonyl]-butyl}-4-morpholin-4-yl-benzamide;
N-{3-methyl-1-[4-(4-methyl-pentanoyl)-6-oxo-hexahydro-pyrrolo[3,2-b]pyrrole-1-carbonyl]-butyl}-4-morpholin-4-yl-benzamide;
N-{1-[4-(2-Amino-4-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Amino-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-pentanoyl)-6-oxo-hexahydro-pyrrolo[3,2-b]pyrrole-1-carbonyl]-3-methyl-butyl}-4-morpholin-4-yl-benzamide;
N-{1-[4-(2-Amino-5-methyl-hexanoyl)-6-oxo-hexahydro-pyrrolo[3,2-b]pyrrole-1-carbonyl]-3-methyl-butyl}-4-morpholin-4-yl-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-morpholin-4-yl-benzamide;
N-{1-[4-(2-hydroxy-3-methyl-butyryl)-6-oxo-hexahydro-pyrrolo[3,2-b]pyrrole-1-carbonyl]-3-methyl-butyl}-4-morpholin-4-yl-benzamide;
N-{1-[4-(2-methoxy-3-methyl-butyryl)-6-oxo-hexahydro-pyrrolo[3,2-b]pyrrole-1-carbonyl]-3-methyl-butyl}-4-morpholin-4-yl-benzamide;
N-{1-[4-(2-hydroxy-3,3-dimethyl-butyryl)-6-oxo-hexahydro-pyrrolo[3,2-b]pyrrole-1-carbonyl]-3-methyl-butyl}-4-morpholin-4-yl-benzamide;
N-{1-[4-(2-methoxy-3,3-dimethyl-butyryl)-6-oxo-hexahydro-pyrrolo[3,2-4b]pyrrole-1-carbonyl]-3-methyl-butyl}-4-morpholin-4-yl-benzamide;
N-{1-[4-(2-hydroxy-3-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-methoxy-3-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-hydroxy-4-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-methoxy-4-methyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-hydroxy-4,4-dimethyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-methoxy-4,4-dimethyl-pentanoyl)-6-oxo-hexahydro-pyrrolo[3,2-b]pyrrole-1-carbonyl]-3-methyl-butyl}-4-morpholin-4-yl-benzamide;
N-{1-[4-(2-hydroxy-pentanoyl)-6-oxo-hexahydro-pyrrolo[3,2-b]pyrrole-1-carbonyl]-3-methyl-butyl}-4-morpholin-4-yl-benzamide;
N-{1-[4-(2-methoxy-pentanoyl)-6-oxo-hexahydro-pyrrolo[3,2-b]pyrrole-1-carbonyl]-3-methyl-butyl}-4-morpholin-4-yl-benzamide;
N-{1-[4-(2-hydroxy-5-methyl-hexanoyl)-6-oxo-hexahydro-pyrrolo[3,2-b]pyrrole-1-carbonyl]-3-methyl-butyl}-4-morpholin-4-yl-benzamide;
N-{1-[4-(2-methoxy-5-methyl-hexanoyl)-6-oxo-hexahydro-pyrrolo[3,2-b]pyrrole-1-carbonyl]-3-methyl-butyl}-4-morpholin-4-yl-benzamide;
N-[1-(4-Benzoyl-6-oxo-hexahydro-pyrrolo[3,2-b]pyrrole-1-carbonyl)-3-methyl-butyl]-4-piperazin-1-ylbenzamide;
N-{3-methyl-1-[6-oxo-4-(pyridine-2-carbonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-2-carbonyl)-hexahydro-pyrrolo[3,2-b]pyrrole-1-carbonyl]-butyl}-4-piperazin-1-yl-benzamide;
N-{3-methyl-1-[6-oxo-4-(pyridine-3-carbonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-3-carbonyl)-hexahydro-pyrrolo[3,2-b]pyrrole-1-carbonyl]-butyl}-4-piperazin-1-yl-benzamide;
N-{3-methyl-1-[6-oxo-4-(pyridine-4-carbonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-4-carbonyl)-hexahydro-pyrrolo[3,2-b]pyrrole-1-carbonyl]-butyl}-4-piperazin-1-yl-benzamide;
N-[1-(4-Benzenesulfonyl-6-oxo-hexahydro-pyrrolo[3,2-b]pyrrole-1-carbonyl)-3-methyl-butyl]-4-piperazin-1-yl-benzamide;
N-{3-methyl-1-[6-oxo-4-(pyridine-2-sulfonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-2-sulfonyl)-hexahydro-pyrrolo[3,2-b]pyrrole-1-carbonyl]-butyl}-4-piperazin-1-yl-benzamide;
N-{3-methyl-1-[6-oxo-4-(pyridine-3-sulfonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-3-sulfonyl)-hexahydro-pyrrolo[3,2-b]pyrrole-1-carbonyl]-butyl}-4-piperazin-1-yl-benzamide;
N-{3-methyl-1-[6-oxo-4-(pyridine-4-sulfonyl)-hexahydro-pyrrolo[3,2-b]pyrrole-1-carbonyl]-butyl}-4-piperazin-1-yl-benzamide;
N-{3-methyl-1-[6-oxo-4-(1-oxy-pyridine-4-sulfonyl)-hexahydro-pyrrolo[3,2-b]pyrrole-1-carbonyl]-butyl}-4-piperazin-1-yl-benzamide;
N-[3-methyl-1-(6-oxo-4-phenylacetyl-hexahydro-pyrrolo[3,2-b]pyrrole-1-carbonyl)-butyl]-4-piperazin-1-yl-benzamide;
4-[2-(4-piperazin-1-yl-benzoylamino)-4-methyl-pentanoyl]-3-oxo-hexahydro-pyrrolo[3,2-b]pyrrole-1-carboxylic acid phenylamide;
4-[2-(4-piperazin-1-yl-benzoylamino)-4-methyl-pentanoyl]-3-oxo-hexahydro-pyrrolo[3,2-b]pyrrole-1-carboxylic acid isobutyl-amide;
4-[2-(4-piperazin-1-yl-benzoylamino)-4-methyl-pentanoyl]-3-oxo-hexahydro-pyrrolo[3,2-b]pyrrole-1-carboxylic acid phenyl ester;
4-[2-(4-piperazin-1-yl-benzoylamino)-4-methyl-pentanoyl]-3-oxo-hexahydro-pyrrolo[3,2-b]pyrrole-1-carboxylic acid isobutyl ester;
N-{3-methyl-1-[6-oxo-4-(pyrrolidine-1-carbonyl)-hexahydro-pyrrolo[3,2-b]pyrrole-1-carbonyl]-butyl}-4-piperazin-1-yl-benzamide;
N-{3-methyl-1-[6-oxo-4-(piperidine-1-carbonyl)-hexahydro-pyrrolo[3,2-b]pyrrole-1-carbonyl]-butyl}-4-piperazin-1-yl-benzamide;
N-{3-methyl-1-[4-(morpholin-4-carbonyl)-6-oxo-hexahydro-pyrrolo[3,2-b]pyrrole-1-carbonyl]-butyl}-4-piperazin-1-yl-benzamide;
N-{3-methyl-1-[6-oxo-4-(piperazine-1-carbonyl)-hexahydro-pyrrolo[3,2-b]pyrrole-1-carbonyl]-butyl}-4-piperazin-1-yl-benzamide;
N-{3-methyl-1-[4-(4-methyl-piperazine-1-carbonyl)-6-oxo-hexahydro-pyrrolo[3,2-b]pyrrole-1-carbonyl]-butyl}-4-piperazin-1-yl-benzamide;
N-(3-methyl-1-{4-[2-(4-methyl-piperazin-1-yl)-acetyl]-6-oxo-hexahydro-pyrrolo[3,2-b]pyrrole-1-carbonyl}-butyl)-4-piperazin-1-yl-benzamide;
N-{3-methyl-1-[6-oxo-4-(2-piperazin-1-yl-acetyl)-hexahydro-pyrrolo[3,2-b]pyrrole-1-carbonyl]-butyl}-4-piperazin-1-yl-benzamide;
N-{1-[4-(furan-2-carbonyl)-6-oxo-hexahydro-pyrrolo[3,2-b]pyrrole-1-carbonyl]-3-methyl-butyl}-4-piperazin-1-yl-benzamide;
N-{1-[4-(5-chloro-furan-2-carbonyl)-6-oxo-hexahydro-pyrrolo[3,2-b]pyrrole-1-carbonyl]-3-methyl-butyl}-4-piperazin-1-yl-benzamide;
N-{1-[4-(thiophene-2-carbonyl)-6-oxo-hexahydro-pyrrolo[3,2-b]pyrrole-1-carbonyl]-3-methyl-butyl}-4-piperazin-1-yl-benzamide;
N-{1-[4-(5-chloro-thiophene-2-carbonyl)-6-oxo-hexahydro-pyrrolo[3,2-b]pyrrole-1-carbonyl]-3-methyl-butyl}-4-piperazin-1-yl-benzamide;
N-{1-[4-(furan-3-carbonyl)-6-oxo-hexahydro-pyrrolo[3,2-b]pyrrole-1-carbonyl]-3-methyl-butyl}-4-piperazin-1-yl-benzamide;
N-{1-[4-(thiophene-3-carbonyl)-6-oxo-hexahydro-pyrrolo[3,2-b]pyrrole-1-carbonyl]-3-methyl-butyl}-4-piperazin-1-yl-benzamide;
N-[1-(4-cyclopentanecarbonyl-6-oxo-hexahydro-pyrrolo[3,2-b]pyrrole-1-carbonyl)-3-methyl-butyl]-4-piperazin-1-yl-benzamide;
N-[1-(4-cyclohexanecarbonyl-6-oxo-hexahydro-pyrrolo[3,2-b]pyrrole-1-carbonyl)-3-methyl-butyl]-4-piperazin-1-yl-benzamide;
N-{1-[4-(cyclopent-3-enecarbonyl)-6-oxo-hexahydro-pyrrolo[3,2-b]pyrrole-1-carbonyl]-3-methyl-butyl}-4-piperazin-1-yl-benzamide;
N-{1-[4-(2-cyclopentyl-acetyl)-6-oxo-hexahydro-pyrrolo[3,2-b]pyrrole-1-carbonyl]-3-methyl-butyl}-4-piperazin-1-yl-benzamide;
N-{1-[4-(2-cyclohexyl-acetyl)-6-oxo-hexahydro-pyrrolo[3,2-b]pyrrole-1-carbonyl]-3-methyl-butyl}-4-piperazin-1-yl-benzamide;
4-piperazin-1-yl-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-piperazin-1-yl-benzamide;
N-{3-methyl-1-[6-oxo-4-(piperidine-2-carbonyl)-hexahydro-pyrrolo[3,2-b]pyrrole-1-carbonyl]-butyl}-4-piperazin-1-yl-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-piperazin-1-yl-benzamide;
N-{3-methyl-1-[6-oxo-4-(tetrahydro-furan-2-carbonyl)-hexahydro-pyrrolo[3,2-b]pyrrole-1-carbonyl]-butyl}-4-piperazin-1-yl-benzamide;
N-{1-[4-(1-Amino-cyclobutanecarbonyl)-6-oxo-hexahydro-pyrrolo[3,2-b]pyrrole-1-carbonyl]-3-methyl-butyl}-4-piperazin-1-yl-benzamide;
N-{1-[4-(1-Amino-cyclopentanecarbonyl)-6-oxo-hexahydro-pyrrolo[3,2-b]pyrrole-1-carbonyl]-3-methyl-butyl}-4-piperazin-1-yl-benzamide;
N-{1-[4-(1-Amino-cyclohexanecarbonyl)-6-oxo-hexahydro-pyrrolo[3,2-b]pyrrole-1-carbonyl]-3-methyl-butyl}-4-piperazin-1-yl-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-piperazin-1-yl-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-piperazin-1-yl-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-piperazin-1-yl-benzamide;
N-{1-[4-(1-hydroxy-cyclobutanecarbonyl)-6-oxo-hexahydro-pyrrolo[3,2-b]pyrrole-1-carbonyl]-3-methyl-butyl}-4-piperazin-1-yl-benzamide;
N-{1-[4-(1-hydroxy-cyclopentanecarbonyl)-6-oxo-hexahydro-pyrrolo[3,2-b]pyrrole-1-carbonyl]-3-methyl-butyl}-4-piperazin-1-yl-benzamide;
N-{1-[4-(1-hydroxy-cyclohexanecarbonyl)-6-oxo-hexahydro-pyrrolo[3,2-b]pyrrole-1-carbonyl]-3-methyl-butyl}-4-piperazin-1-yl-benzamide;
N-{1-[4-(1-methoxy-cyclobutanecarbonyl)-6-oxo-hexahydro-pyrrolo[3,2-b]pyrrole-1-carbonyl]-3-methyl-butyl}-4-piperazin-1-yl-benzamide;
N-{1-[4-(1-methoxy-cyclopentanecarbonyl)-6-oxo-hexahydro-pyrrolo[3,2-b]pyrrole-1-carbonyl]-3-methyl-butyl}-4-piperazin-1-yl-benzamide;
N-{1-[4-(1-methoxy-cyclohexanecarbonyl)-6-oxo-hexahydro-pyrrolo[3,2-b]pyrrole-1-carbonyl]-3-methyl-butyl}-4-piperazin-1-yl-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-piperazin-1-yl-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-propionyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-acetyl)-6-oxo-hexahydro-pyrrolo[3,2-b]pyrrole-1-carbonyl]-3-methyl-butyl}-4-piperazin-1-yl-benzamide;
N-(1-{4-[2-(Acetyl-methyl-amino)-acetyl]-6-oxo-hexahydro-pyrrolo[3,2-b]pyrrole-1-carbonyl}-3-methyl-butyl)-4-piperazin-1-yl-benzamide;
N-{1-[4-(2-dimethylamino-acetyl)-6-oxo-hexahydro-pyrrolo[3,2-b]pyrrole-1-carbonyl]-3-methyl-butyl}-4-piperazin-1-yl-benzamide;
N-{1-[4-(2-Amino-butyryl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-butyryl)-6-oxo-hexahydro-pyrrolo[3,2-b]pyrrole-1-carbonyl]-3-methyl-butyl}-4-piperazin-1-yl-benzamide;
N-{1-[4-(2-amino-3-methyl-butyryl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-piperazin-1-yl-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-piperazin-1-yl-benzamide;
N-{1-[4-(2-Amino-3-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-piperazin-1-yl-benzamide;
N-[3-methyl-1-(6-oxo-4-pentanoyl-hexahydro-pyrrolo[3,2-b]pyrrole-1-carbonyl)-butyl]-4-piperazin-1-yl-benzamide;
N-{3-methyl-1-[4-(3-methyl-pentanoyl)-6-oxo-hexahydro-pyrrolo[3,2-b]pyrrole-1-carbonyl]-butyl}-4-piperazin-1-yl-benzamide;
N-{3-methyl-1-[4-(4-methyl-pentanoyl)-6-oxo-hexahydro-pyrrolo[3,2-b]pyrrole-1-carbonyl]-butyl}-4-piperazin-1-yl-benzamide;
N-{1-[4-(2-Amino-4-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Amino-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-pentanoyl)-6-oxo-hexahydro-pyrrolo[3,2-b]pyrrole-1-carbonyl]-3-methyl-butyl}-4-piperazin-1-yl-benzamide;
N-{1-[4-(2-Amino-5-methyl-hexanoyl)-6-oxo-hexahydro-pyrrolo[3,2-b]pyrrole-1-carbonyl]-3-methyl-butyl}-4-piperazin-1-yl-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-piperazin-1-yl-benzamide;
N-{1-[4-(2-hydroxy-3-methyl-butyryl)-6-oxo-hexahydro-pyrrolo[3,2-b]pyrrole-1-carbonyl]-3-methyl-butyl}-4-piperazin-1-yl-benzamide;
N-{1-[4-(2-methoxy-3-methyl-butyryl)-6-oxo-hexahydro-pyrrolo[3,2-b]pyrrole-1-carbonyl]-3-methyl-butyl}-4-piperazin-1-yl-benzamide;
N-{1-[4-(2-hydroxy-3,3-dimethyl-butyryl)-6-oxo-hexahydro-pyrrolo[3,2-b]pyrrole-1-carbonyl]-3-methyl-butyl}-4-piperazin-1-yl-benzamide;
N-{1-[4-(2-methoxy-3,3-dimethyl-butyryl)-6-oxo-hexahydro-pyrrolo[3,2-b]pyrrole-1-carbonyl]-3-methyl-butyl}-4-piperazin-1-yl-benzamide;
N-{1-[4-(2-hydroxy-3-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-methoxy-3-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-hydroxy-4-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-methoxy-4-methyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-hydroxy-4,4-dimethyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-methoxy-4,4-dimethyl-pentanoyl)-6-oxo-hexahydro-pyrrolo[3,2-b]pyrrole-1-carbonyl]-3-methyl-butyl}-4-piperazin-1-yl-benzamide;
N-{1-[4-(2-hydroxy-pentanoyl)-6-oxo-hexahydro-pyrrolo[3,2-b]pyrrole-1-carbonyl]-3-methyl-butyl}-4-piperazin-1-yl-benzamide;
N-{1-[4-(2-methoxy-pentanoyl)-6-oxo-hexahydro-pyrrolo[3,2-b]pyrrole-1-carbonyl]-3-methyl-butyl}-4-piperazin-1-yl-benzamide;
N-{1-[4-(2-hydroxy-5-methyl-hexanoyl)-6-oxo-hexahydro-pyrrolo[3,2-b]pyrrole-1-carbonyl]-3-methyl-butyl}-4-piperazin-1-yl-benzamide;
N-{1-[4-(2-methoxy-5-methyl-hexanoyl)-6-oxo-hexahydro-pyrrolo[3,2-b]pyrrole-1-carbonyl]-3-methyl-butyl}-4-piperazin-1-yl-benzamide;
N-[1-(4-Benzoyl-6-oxo-hexahydro-pyrrolo[3,2-b]pyrrole-1-carbonyl)-3-methyl-butyl]-4-(4-methyl-piperazin-1-yl)benzamide;
N-{3-methyl-1-[6-oxo-4-(1-oxy-pyridine-2-carbonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(pyridine-3-carbonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(1-oxy-pyridine-3-carbonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(pyridine-4-carbonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(1-oxy-pyridine-4-carbonyl)-hexahydro-pyrrolo[3,2-b]pyrrole-1-carbonyl]-butyl}-4-(4-methyl-piperazin-1-yl)-benzamide;
N-[1-(4-Benzenesulfonyl-6-oxo-hexahydro-pyrrolo[3,2-b]pyrrole-1-carbonyl)-3-methyl-butyl]-4-(4-methyl-piperazin-1-yl)-benzamide;
N-{3-methyl-1-[6-oxo-4-(pyridine-4-sulfonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(1-oxy-pyridine-4-sulfonyl)-hexahydro-pyrrolo[3,2-b]pyrrole-1-carbonyl]-butyl}-4-(4-methyl-piperazin-1-yl)-benzamide;
N-[3-methyl-1-(6-oxo-4-phenylacetyl-hexahydro-pyrrolo[3,2-b]pyrrole-1-carbonyl)-butyl]-4-(4-methyl-piperazin-1-yl)-benzamide;
4-[2-(4-(4-methyl-piperazin-1-yl)-benzoylamino)-4-methyl-pentanoyl]-3-oxo-hexahydro-pyrrolo[3,2-b]pyrrole-1-carboxylic acid phenylamide;
4-[2-(4-(4-methyl-piperazin-1-yl)-benzoylamino)-4-methyl-pentanoyl]-3-oxo-hexahydro-pyrrolo[3,2-b]pyrrole-1-carboxylic acid isobutyl-amide;
4-[2-(4-(4-methyl-piperazin-1-yl)-benzoylamino)-4-methyl-pentanoyl]-3-oxo-hexahydro-pyrrolo[3,2-b]pyrrole-1-carboxylic acid phenyl ester;
4-[2-(4-(4-methyl-piperazin-1-yl)-benzoylamino)-4-methyl-pentanoyl]-3-oxo-hexahydro-pyrrolo[3,2-b]pyrrole-1-carboxylic acid isobutyl ester;
N-{3-methyl-1-[6-oxo-4-(pyrrolidine-1-carbonyl)-hexahydro-pyrrolo[3,2-b]pyrrole-1-carbonyl]-butyl}-4-(4-methyl-piperazin-1-yl)-benzamide;
N-{3-methyl-1-[6-oxo-4-(piperidine-1-carbonyl)-hexahydro-pyrrolo[3,2-b]pyrrole-1-carbonyl]-butyl}-4-(4-methyl-piperazin-1-yl)-benzamide;
N-{3-methyl-1-[4-(morpholin-4-carbonyl)-6-oxo-hexahydro-pyrrolo[3,2-b]pyrrole-1-carbonyl]-butyl}-4-(4-methyl-piperazin-1-yl)-benzamide;
N-{3-methyl-1-[6-oxo-4-(piperazine-1-carbonyl)-hexahydro-pyrrolo[3,2-b]pyrrole-1-carbonyl]-butyl}-4-(4-methyl-piperazin-1-yl)-benzamide;
N-{3-methyl-1-[4-(4-methyl-piperazine-1-carbonyl)-6-oxo-hexahydro-pyrrolo[3,2-b]pyrrole-1-carbonyl]-butyl}-4-(4-methyl-piperazin-1-yl)-benzamide;
N-(3-methyl-1-{4-[2-(4-methyl-piperazin-1-yl)-acetyl]-6-oxo-hexahydro-pyrrolo[3,2-b]pyrrole-1-carbonyl}-butyl]-4-(4-methyl-piperazin-1-yl)-benzamide;
N-{3-methyl-1-[6-oxo-4-(2-piperazin-1-yl-acetyl)-hexahydro-pyrrolo[3,2-b]pyrrole-1-carbonyl]-butyl}-4-(4-methyl-piperazin-1-yl)-benzamide;
N-{1-[4-(furan-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(5-chloro-furan-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(thiophene-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(5-chloro-thiophene-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(furan-3-carbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(thiophene-3-carbonyl)-6-oxo-hexahydro-pyrrolo[3,2-b]pyrrole-1-carbonyl]-3-methyl-butyl}-4-(4-methyl-piperazin-1-yl)-benzamide;
N-[1-(4-cyclopentanecarbonyl-6-oxo-hexahydro-pyrrolo[3,2-b]pyrrole-1-carbonyl)-3-methyl-butyl]-4-(4-methyl-piperazin-1-yl)-benzamide;
N-[1-(4-cyclohexanecarbonyl-6-oxo-hexahydro-pyrrolo[3,2-b]pyrrole-1-carbonyl)-3-methyl-butyl]-4-(4-methyl-piperazin-1-yl)-benzamide;
N-{1-[4-(cyclopent-3-enecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
4-(4-methyl-piperazin-1-yl)-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{3-methyl-1-[6-oxo-4-(piperidine-2-carbonyl)-hexahydro-pyrrolo[3,2-b]pyrrole-1-carbonyl]-butyl}-4-(4-methyl-piperazin-1-yl)-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-(4-methyl-piperazin-1-yl)-benzamide;
N-{3-methyl-1-[6-oxo-4-(tetrahydro-furan-2-carbonyl)-hexahydro-pyrrolo[3,2-b]pyrrole-1-carbonyl]-butyl}-4-(4-methyl-piperazin-1-yl)-benzamide;
N-{1-[4-(1-Amino-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-Amino-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-Amino-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-hydroxy-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-hydroxy-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-hydroxy-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-methoxy-cyclobutanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-methoxy-cyclopentanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(1-methoxy-cyclohexanecarbonyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-propionyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-acetyl)-6-oxo-hexahydro-pyrrolo[3,2-b]pyrrole-1-carbonyl]-3-methyl-butyl}-4-(4-methyl-piperazin-1-yl)-benzamide;
N-(1-{4-[2-(Acetyl-methyl-amino)-acetyl]-6-oxo-hexahydro-pyrrolo[3,2-b]pyrrole-1-carbonyl}-3-methyl-butyl)-4-(4-methyl-piperazin-1-yl)-benzamide;
N-{1-[4-(2-dimethylamino-acetyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-amino-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-[3-methyl-1-(6-oxo-4-pentanoyl-hexahydro-pyrrolo[3,2-b]pyrrole-1-carbonyl)-butyl]-4-(4-methyl-piperazin-1-yl)-benzamide;
N-{3-methyl-1-[4-(3-methyl-pentanoyl)-6-oxo-hexahydro-pyrrolo[3,2-b]pyrrole-1-carbonyl]-butyl}-4-(4-methyl-piperazin-1-yl)-benzamide;
N-{3-methyl-1-[4-(4-methyl-pentanoyl)-6-oxo-hexahydro-pyrrolo[3,2-b]pyrrole-1-carbonyl]-butyl}-4-(4-methyl-piperazin-1-yl)-benzamide;
N-{1-[4-(2-Amino-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Amino-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-3-methyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-3,3-dimethyl-butyryl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-3-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-4-methyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-4,4-dimethyl-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-pentanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-hydroxy-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
N-{1-[4-(2-methoxy-5-methyl-hexanoyl)-6-oxo-hexahydro-pyrrolo[3,2-b]pyrrole-1-carbonyl]-3-methyl-butyl}-4-(4-methyl-piperazin-1-yl)-benzamide;
N-[1-(4-Benzoyl-6-oxo-hexahydro-pyrrolo[3,2-b]pyrrole-1-carbonyl)-3-methyl-butyl]-4-aminobenzamide;
4-Amino-N-{3-methyl-1-[6-oxo-4-(pyridine-2-carbonyl)-hexahydro-pyrrolo[3,2-b]pyrrole-1-carbonyl]-butyl}-benzamide;
4-Amino-N-{3-methyl-1-[6-oxo-4-(1-oxy-pyridine-2-carbonyl)-hexahydro-pyrrolo[3,2-b]pyrrole-1-carbonyl]-butyl}-benzamide;
4-Amino-N-{3-methyl-1-[6-oxo-4-(pyridine-3-carbonyl)-hexahydro-pyrrolo[3,2-b]pyrrole-1-carbonyl]-butyl}-benzamide;
4-Amino-N-{3-methyl-1-[6-oxo-4-(1-oxy-pyridine-3-carbonyl)-hexahydro-pyrrolo[3,2-b]pyrrole-1-carbonyl]-butyl}-benzamide;
4-Amino-N-{3-methyl-1-[6-oxo-4-(pyridine-4-carbonyl)-hexahydro-pyrrolo[3,2-b]pyrrole-1-carbonyl]-butyl}-benzamide;
4-Amino-N-{3-methyl-1-[6-oxo-4-(1-oxy-pyridine-4-carbonyl)-hexahydro-pyrrolo[3,2-b]pyrrole-1-carbonyl]-butyl}-benzamide;
N-[1-(4-Benzenesulfonyl-6-oxo-hexahydro-pyrrolo[3,2-b]pyrrole-1-carbonyl)-3-methyl-butyl]-4-amino-benzamide;
4-Amino-N-{3-methyl-1-[6-oxo-4-(pyridine-2-sulfonyl)-hexahydro-pyrrolo[3,2-b]pyrrole-1-carbonyl]-butyl}-benzamide;
4-Amino-N-{3-methyl-1-[6-oxo-4-(1-oxy-pyridine-2-sulfonyl)-hexahydro-pyrrolo[3,2-b]pyrrole-1-carbonyl]-butyl}-benzamide;
4-Amino-N-{3-methyl-1-[6-oxo-4-(pyridine-3-sulfonyl)-hexahydro-pyrrolo[3,2-b]pyrrole-1-carbonyl]-butyl}-benzamide;
4-Amino-N-{3-methyl-1-[6-oxo-4-(1-oxy-pyridine-3-sulfonyl)-hexahydro-pyrrolo[3,2-b]pyrrole-1-carbonyl]-butyl}-benzamide;
4-Amino-N-{3-methyl-1-[6-oxo-4-(pyridine-4-sulfonyl)-hexahydro-pyrrolo[3,2-b]pyrrole-1-carbonyl]-butyl}-benzamide;
4-Amino-N-{3-methyl-1-[6-oxo-4-(1-oxy-pyridine-4-sulfonyl)-hexahydro-pyrrolo[3,2-b]pyrrole-1-carbonyl]-butyl}-benzamide;
4-Amino-N-[3-methyl-1-(6-oxo-4-phenylacetyl-hexahydro-pyrrolo[3,2-b]pyrrole-1-carbonyl)-butyl]-benzamide;
4-[2-(4-amino-benzoylamino)-4-methyl-pentanoyl]-3-oxo-hexahydro-pyrrolo[3,2-b]pyrrole-1-carboxylic acid phenylamide;
4-[2-(4-amino-benzoylamino)-4-methyl-pentanoyl]-3-oxo-hexahydro-pyrrolo[3,2-b]pyrrole-1-carboxylic acid isobutyl-amide;
4-[2-(4-amino-benzoylamino)-4-methyl-pentanoyl]-3-oxo-hexahydro-pyrrolo[3,2-b]pyrrole-1-carboxylic acid phenyl ester;
4-[2-(4-amino-benzoylamino)-4-methyl-pentanoyl]-3-oxo-hexahydro-pyrrolo[3,2-b]pyrrole-1-carboxylic acid isobutyl ester;
4-Amino-N-{3-methyl-1-[6-oxo-4-(pyrrolidine-1-carbonyl)-hexahydro-pyrrolo[3,2-b]pyrrole-1-carbonyl]-butyl}-benzamide;
4-Amino-N-{3-methyl-1-[6-oxo-4-(piperidine-1-carbonyl)-hexahydro-pyrrolo[3,2-b]pyrrole-1-carbonyl]-butyl}-benzamide;
4-Amino-N-{3-methyl-1-[4-(morpholin-4-carbonyl)-6-oxo-hexahydro-pyrrolo[3,2-b]pyrrole-1-carbonyl]-butyl}-benzamide;
4-Amino-N-{3-methyl-1-[6-oxo-4-(piperazine-1-carbonyl)-hexahydro-pyrrolo[3,2-b]pyrrole-1-carbonyl]-butyl}-benzamide;
4-Amino-N-{3-methyl-1-[4-(4-methyl-piperazine-1-carbonyl)-6-oxo-hexahydro-pyrrolo[3,2-b]pyrrole-1-carbonyl]-butyl}-benzamide;
4-Amino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
4-Amino-N-{3-methyl-1-[6-oxo-4-(2-piperazin-1-yl-acetyl)-hexahydro-pyrrolo[3,2-b]pyrrole-1-carbonyl]-butyl}-benzamide;
4-Amino-N-{1-[4-(furan-2-carbonyl)-6-oxo-hexahydro-pyrrolo[3,2-b]pyrrole-1-carbonyl]-3-methyl-butyl}-benzamide;
4-Amino-N-{1-[4-(5-chloro-furan-2-carbonyl)-6-oxo-hexahydro-pyrrolo[3,2-b]pyrrole-1-carbonyl]-3-methyl-butyl}-benzamide;
4-Amino-N-{1-[4-(thiophene-2-carbonyl)-6-oxo-hexahydro-pyrrolo[3,2-b]pyrrole-1-carbonyl]-3-methyl-butyl}-benzamide;
4-Amino-N-{1-[4-(5-chloro-thiophene-2-carbonyl)-6-oxo-hexahydro-pyrrolo[3,2-b]pyrrole-1-carbonyl]-3-methyl-butyl}-benzamide;
4-Amino-N-{1-[4-(furan-3-carbonyl)-6-oxo-hexahydro-pyrrolo[3,2-b]pyrrole-1-carbonyl]-3-methyl-butyl}-benzamide;
4-Amino-N-{1-[4-(thiophene-3-carbonyl)-6-oxo-hexahydro-pyrrolo[3,2-b]pyrrole-1-carbonyl]-3-methyl-butyl}-benzamide;
4-Amino-N-[1-(4-cyclopentanecarbonyl-6-oxo-hexahydro-pyrrolo[3,2-b]pyrrole-1-carbonyl)-3-methyl-butyl]-benzamide;
4-Amino-N-[1-(4-cyclohexanecarbonyl-6-oxo-hexahydro-pyrrolo[3,2-b]pyrrole-1-carbonyl)-3-methyl-butyl]-benzamide;
4-Amino-N-{1-[4-(cyclopent-3-enecarbonyl)-6-oxo-hexahydro-pyrrolo[3,2-b]pyrrole-1-carbonyl]-3-methyl-butyl}-benzamide;
4-Amino-N-{1-[4-(2-cyclopentyl-acetyl)-6-oxo-hexahydro-pyrrolo[3,2-b]pyrrole-1-carbonyl]-3-methyl-butyl}-benzamide;
4-Amino-N-{1-[4-(2-cyclohexyl-acetyl)-6-oxo-hexahydro-pyrrolo[3,2-b]pyrrole-1-carbonyl]-3-methyl-butyl}-benzamide;
4-Amino-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-amino-benzamide;
4-Amino-N-{3-methyl-1-[6-oxo-4-(piperidine-2-carbonyl)-hexahydro-pyrrolo[3,2-b]pyrrole-1-carbonyl]-butyl}-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-amino-benzamide;
4-Amino-N-{3-methyl-1-[6-oxo-4-(tetrahydro-furan-2-carbonyl)-hexahydro-pyrrolo[3,2-b]pyrrole-1-carbonyl]-butyl}-benzamide;
N-{1-[4-(1-Amino-cyclobutanecarbonyl)-6-oxo-hexahydro-pyrrolo[3,2-b]pyrrole-1-carbonyl]-3-methyl-butyl}-4-Amino-benzamide;
N-{1-[4-(1-Amino-cyclopentanecarbonyl)-6-oxo-hexahydro-pyrrolo[3,2-b]pyrrole-1-carbonyl]-3-methyl-butyl}-4-amino-benzamide;
N-{1-[4-(1-Amino-cyclohexanecarbonyl)-6-oxo-hexahydro-pyrrolo[3,2-b]pyrrole-1-carbonyl]-3-methyl-butyl}-4-amino-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-amino-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-amino-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-amino-benzamide;
4-Amino-N-{1-[4-(1-hydroxy-cyclobutanecarbonyl)-6-oxo-hexahydro-pyrrolo[3,2-b]pyrrole-1-carbonyl]-3-methyl-butyl}-benzamide;
4-Amino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
4-Amino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
4-Amino-N-{1-[4-(1-methoxy-cyclobutanecarbonyl)-6-oxo-hexahydro-pyrrolo[3,2-b]pyrrole-1-carbonyl]-3-methyl-butyl}-benzamide;
4-Amino-N-{1-[4-(1-methoxy-cyclopentanecarbonyl)-6-oxo-hexahydro-pyrrolo[3,2-b]pyrrole-1-carbonyl]-3-methyl-butyl}-benzamide;
4-Amino-N-{1-[4-(1-methoxy-cyclohexanecarbonyl)-6-oxo-hexahydro-pyrrolo[3,2-b]pyrrole-1-carbonyl]-3-methyl-butyl}-benzamide;
N-{1-[4-(2-amino-2-cyclopentyl-acetyl)-6-oxo-hexahydro-pyrrolo[3,2-b]pyrrole-1-carbonyl]-3-methyl-butyl}-4-amino-benzamide;
N-{1-[4-(2-amino-2-cyclohexyl-acetyl)-6-oxo-hexahydro-pyrrolo[3,2-b]pyrrole-1-carbonyl]-3-methyl-butyl}-4-amino-benzamide;
N-{1-[4-(2-acetylamino-2-cyclopentyl-acetyl)-6-oxo-hexahydro-pyrrolo[3,2-b]pyrrole-1-carbonyl]-3-methyl-butyl}-4-amino-benzamide;
N-{1-[4-(2-acetylamino-2-cyclohexyl-acetyl)-6-oxo-hexahydro-pyrrolo[3,2-b]pyrrole-1-carbonyl]-3-methyl-butyl}-4-amino-benzamide;
N-{1-[4-(2-acetylamino-propionyl)-6-oxo-hexahydro-pyrrolo[3,2-b]pyrrole-1-carbonyl]-3-methyl-butyl}-4-amino-benzamide;
N-{1-[4-(2-acetylamino-acetyl)-6-oxo-hexahydro-pyrrolo[3,2-b]pyrrole-1-carbonyl]-3-methyl-butyl}-4-amino-benzamide;
N-(1-{4-[2-(acetyl-methyl-amino)-acetyl]-6-oxo-hexahydro-pyrrolo[3,2-b]pyrrole-1-carbonyl}-3-methyl-butyl)-4-amino-benzamide;
4-Amino-N-{1-[4-(2-dimethylamino-acetyl)-6-oxo-hexahydro-pyrrolo[3,2-b]pyrrole-1-carbonyl]-3-methyl-butyl}-benzamide;
N-{1-[4-(2-amino-butyryl)-6-oxo-hexahydro-pyrrolo[3,2-b]pyrrole-1-carbonyl]-3-methyl-butyl}-4-amino-benzamide;
N-{1-[4-(2-acetylamino-butyryl)-6-oxo-hexahydro-pyrrolo[3,2-b]pyrrole-1-carbonyl]-3-methyl-butyl}-4-amino-benzamide;
N-{1-[4-(2-amino-3-methyl-butyryl)-6-oxo-hexahydro-pyrrolo[3,2-b]pyrrole-1-carbonyl]-3-methyl-butyl}-4-amino-benzamide;
N-{1-[4-(2-acetylamino-3-methyl-butyryl)-6-oxo-hexahydro-pyrrolo[3,2-b]pyrrole-1-carbonyl]-3-methyl-butyl}-4-amino-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-amino-benzamide;
N-{1-[4-(2-acetylamino-3,3-dimethyl-butyryl)-6-oxo-hexahydro-pyrrolo[3,2-b]pyrrole-1-carbonyl]-3-methyl-butyl}-4-amino-benzamide;
N-{1-[4-(2-amino-3-methyl-pentanoyl)-6-oxo-hexahydro-pyrrolo[3,2-b]pyrrole-1-carbonyl]-3-methyl-butyl}-4-amino-benzamide;
N-{1-[4-(2-acetylamino-3-methyl-pentanoyl)-6-oxo-hexahydro-pyrrolo[3,2-b]pyrrole-1-carbonyl]-3-methyl-butyl}-4-amino-benzamide;
4-Amino-N-[3-methyl-1-(6-oxo-4-pentanoyl-hexahydro-pyrrolo[3,2-b]pyrrole-1-carbonyl)-butyl]-benzamide;
4-Amino-N-{3-methyl-1-[4-(3-methyl-pentanoyl)-6-oxo-hexahydro-pyrrolo[3,2-b]pyrrole-1-carbonyl]-butyl}-benzamide;
4-Amino-N-{3-methyl-1-[4-(4-methyl-pentanoyl)-6-oxo-hexahydro-pyrrolo[3,2-b]pyrrole-1-carbonyl]-butyl}-benzamide;
N-{1-[4-(2-amino-4-methyl-pentanoyl)-6-oxo-hexahydro-pyrrolo[3,2-b]pyrrole-1-carbonyl]-3-methyl-butyl}-4-amino-benzamide;
N-{1-[4-(2-acetylamino-4-methyl-pentanoyl)-6-oxo-hexahydro-pyrrolo[3,2-b]pyrrole-1-carbonyl]-3-methyl-butyl}-4-amino-benzamide;
N-{1-[4-(2-amino-4,4-dimethyl-pentanoyl)-6-oxo-hexahydro-pyrrolo[3,2-b]pyrrole-1-carbonyl]-3-methyl-butyl}-4-amino-benzamide;
N-{1-[4-(2-acetylamino-4,4-dimethyl-pentanoyl)-6-oxo-hexahydro-pyrrolo[3,2-b]pyrrole-1-carbonyl]-3-methyl-butyl}-4-amino-benzamide;
N-{1-[4-(2-amino-pentanoyl)-6-oxo-hexahydro-pyrrolo[3,2-b]pyrrole-1-carbonyl]-3-methyl-butyl}-4-amino-benzamide;
N-{1-[4-(2-acetylamino-pentanoyl)-6-oxo-hexahydro-pyrrolo[3,2-b]pyrrole-1-carbonyl]-3-methyl-butyl}-4-amino-benzamide;
N-{1-[4-(2-amino-5-methyl-hexanoyl)-6-oxo-hexahydro-pyrrolo[3,2-b]pyrrole-1-carbonyl]-3-methyl-butyl}-4-amino-benzamide;
N-{1-[4-(2-acetylamino-5-methyl-hexanoyl)-6-oxo-hexahydro-pyrrolo[3,2-b]pyrrole-1-carbonyl]-3-methyl-butyl}-4-amino-benzamide;
N-{1-[4-(2-hydroxy-3-methyl-butyryl)-6-oxo-hexahydro-pyrrolo[3,2-b]pyrrole-1-carbonyl]-3-methyl-butyl}-4-amino-benzamide;
N-{1-[4-(2-methoxy-3-methyl-butyryl)-6-oxo-hexahydro-pyrrolo[3,2-b]pyrrole-1-carbonyl]-3-methyl-butyl}-4-amino-benzamide;
4-Amino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
4-Amino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
4-Amino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
4-Amino-N-{1-[4-(2-methoxy-3-methyl-pentanoyl)-6-oxo-hexahydro-pyrrolo[3,2-b]pyrrole-1-carbonyl]-3-methyl-butyl}-benzamide;
4-Amino-N-{1-[4-(2-hydroxy-4-methyl-pentanoyl)-6-oxo-hexahydro-pyrrolo[3,2-b]pyrrole-1-carbonyl]-3-methyl-butyl}-benzamide;
4-Amino-N-{1-[4-(2-methoxy-4-methyl-pentanoyl)-6-oxo-hexahydro-pyrrolo[3,2-b]pyrrole-1-carbonyl]-3-methyl-butyl}-benzamide;
4-Amino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
4-Amino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
4-Amino-N-{1-[4-(2-hydroxy-pentanoyl)-6-oxo-hexahydro-pyrrolo[3,2-b]pyrrole-1-carbonyl]-3-methyl-butyl}-benzamide;
4-Amino-N-{1-[4-(2-methoxy-pentanoyl)-6-oxo-hexahydro-pyrrolo[3,2-b]pyrrole-1-carbonyl]-3-methyl-butyl}-benzamide;
4-Amino-N-{1-[4-(2-hydroxy-5-methyl-hexanoyl)-6-oxo-hexahydro-pyrrolo[3,2-b]pyrrole-1-carbonyl]-3-methyl-butyl}-benzamide;
4-Amino-N-{1-[4-(2-methoxy-5-methyl-hexanoyl)-6-oxo-hexahydro-pyrrolo[3,2-b]pyrrole-1-carbonyl]-3-methyl-butyl}-benzamide;
N-[1-(4-Benzoyl-6-oxo-hexahydro-pyrrolo[3,2-b]pyrrole-1-carbonyl)-3-methyl-butyl]-4-diethylaminobenzamide;
4-Diethylamino-N-{3-methyl-1-[6-oxo-4-(pyridine-2-carbonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-2-carbonyl)-hexahydro-pyrrolo[3,2-b]pyrrole-1-carbonyl]-butyl}-benzamide;
4-Diethylamino-N-{3-methyl-1-[6-oxo-4-(pyridine-3-carbonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-3-carbonyl)-hexahydro-pyrrolo[3,2-b]pyrrole-1-carbonyl]-butyl}-benzamide;
4-Diethylamino-N-{3-methyl-1-[6-oxo-4-(pyridine-4-carbonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-4-carbonyl)-hexahydro-pyrrolo[3,2-b]pyrrole-1-carbonyl]-butyl}-benzamide;
N-[1-(4-Benzenesulfonyl-6-oxo-hexahydro-pyrrolo[3,2-b]pyrrole-1-carbonyl)-3-methyl-butyl]-4-diethylamino-benzamide;
4-Diethylamino-N-{3-methyl-1-[6-oxo-4-(pyridine-2-sulfonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-2-sulfonyl)-hexahydro-pyrrolo[3,2-b]pyrrole-1-carbonyl]-butyl}-benzamide;
4-Diethylamino-N-{3-methyl-1-[6-oxo-4-(pyridine-3-sulfonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-3-sulfonyl)-hexahydro-pyrrolo[3,2-b]pyrrole-1-carbonyl]-butyl}-benzamide;
4-Diethylamino-N-{3-methyl-1-[6-oxo-4-(pyridine-4-sulfonyl)-hexahydro-pyrrolo[3,2-b]pyrrole-1-carbonyl]-butyl}-benzamide;
4-Diethylamino-N-{3-methyl-1-[6-oxo-4-(1-oxy-pyridine-4-sulfonyl)-hexahydro-pyrrolo[3,2-b]pyrrole-1-carbonyl]-butyl}-benzamide;
4-Diethylamino-N-[3-methyl-1-(6-oxo-4-phenylacetyl-hexahydro-pyrrolo[3,2-b]pyrrole-1-carbonyl)-butyl]-benzamide;
4-[2-(4-diethylamino-benzoylamino)-4-methyl-pentanoyl]-3-oxo-hexahydro-pyrrolo[3,2-b]pyrrole-1-carboxylic acid phenylamide;
4-[2-(4-diethylamino-benzoylamino)-4-methyl-pentanoyl]-3-oxo-hexahydro-pyrrolo[3,2-b]pyrrole-1-carboxylic acid isobutyl-amide;
4-[2-(4-diethylamino-benzoylamino)-4-methyl-pentanoyl]-3-oxo-hexahydro-pyrrolo[3,2-b]pyrrole-1-carboxylic acid phenyl ester;
4-[2-(4-diethylamino-benzoylamino)-4-methyl-pentanoyl]-3-oxo-hexahydro-pyrrolo[3,2-b]pyrrole-1-carboxylic acid isobutyl ester;
4-Diethylamino-N-{3-methyl-1-[6-oxo-4-(pyrrolidine-1-carbonyl)-hexahydro-pyrrolo[3,2-b]pyrrole-1-carbonyl]-butyl}-benzamide;
4-Diethylamino-N-{3-methyl-1-[6-oxo-4-(piperidine-1-carbonyl)-hexahydro-pyrrolo[3,2-b]pyrrole-1-carbonyl]-butyl}-benzamide;
4-Diethylamino-N-{3-methyl-1-[4-(morpholin-4-carbonyl)-6-oxo-hexahydro-pyrrolo[3,2-b]pyrrole-1-carbonyl]-butyl}-benzamide;
4-Diethylamino-N-{3-methyl-1-[6-oxo-4-(piperazine-1-carbonyl)-hexahydro-pyrrolo[3,2-b]pyrrole-1-carbonyl]-butyl}-benzamide;
4-Diethylamino-N-{3-methyl-1-[4-(4-methyl-piperazine-1-carbonyl)-6-oxo-hexahydro-pyrrolo[3,2-b]pyrrole-1-carbonyl]-butyl}-benzamide;
4-Diethylamino-N-(3-methyl-1-{4-[2-(4-methyl-piperazin-1-yl)-acetyl]-6-oxo-hexahydro-pyrrolo[3,2-b]pyrrole-1-carbonyl}-butyl)-benzamide;
4-Diethylamino-N-{3-methyl-1-[6-oxo-4-(2-piperazin-1-yl-acetyl)-hexahydro-pyrrolo[3,2-b]pyrrole-1-carbonyl]-butyl}-benzamide;
4-Diethylamino-N-{1-[4-(furan-2-carbonyl)-6-oxo-hexahydro-pyrrolo[3,2-b]pyrrole-1-carbonyl]-3-methyl-butyl}-benzamide;
4-Diethylamino-N-{1-[4-(5-chloro-furan-2-carbonyl)-6-oxo-hexahydro-pyrrolo[3,2-b]pyrrole-1-carbonyl]-3-methyl-butyl}-benzamide;
4-Diethylamino-N-{1-[4-(thiophene-2-carbonyl)-6-oxo-hexahydro-pyrrolo[3,2-b]pyrrole-1-carbonyl]-3-methyl-butyl}-benzamide;
4-Diethylamino-N-{1-[4-(5-chloro-thiophene-2-carbonyl)-6-oxo-hexahydro-pyrrolo[3,2-b]pyrrole-1-carbonyl]-3-methyl-butyl}-benzamide;
4-Diethylamino-N-{1-[4-(furan-3-carbonyl)-6-oxo-hexahydro-pyrrolo[3,2-b]pyrrole-1-carbonyl]-3-methyl-butyl}-benzamide;
4-Diethylamino-N-{1-[4-(thiophene-3-carbonyl)-6-oxo-hexahydro-pyrrolo[3,2-b]pyrrole-1-carbonyl]-3-methyl-butyl}-benzamide;
4-Diethylamino-N-[1-(4-cyclopentanecarbonyl-6-oxo-hexahydro-pyrrolo[3,2-b]pyrrole-1-carbonyl)-3-methyl-butyl]-benzamide;
4-Diethylamino-N-[1-(4-cyclohexanecarbonyl-6-oxo-hexahydro-pyrrolo[3,2-b]pyrrole-1-carbonyl)-3-methyl-butyl]-benzamide;
4-Diethylamino-N-{1-[4-(cyclopent-3-enecarbonyl)-6-oxo-hexahydro-pyrrolo[3,2-b]pyrrole-1-carbonyl]-3-methyl-butyl}-benzamide;
4-Diethylamino-N-{1-[4-(2-cyclopentyl-acetyl)-6-oxo-hexahydro-pyrrolo[3,2-b]pyrrole-1-carbonyl]-3-methyl-butyl}-benzamide;
4-Diethylamino-N-{1-[4-(2-cyclohexyl-acetyl)-6-oxo-hexahydro-pyrrolo[3,2-b]pyrrole-1-carbonyl]-3-methyl-butyl}-benzamide;
4-Diethylamino-N-{3-methyl-1-[6-oxo-4-(pyrrolidine-2-carbonyl)-hexahydro-pyrrolo[3,2-b]pyrrole-1-carbonyl]-butyl}-benzamide;
N-{1-[4-(1-Acetyl-pyrrolidine-2-carbonyl)-6-oxo-hexahydro-pyrrolo[3,2-b]pyrrole-1-carbonyl]-3-methyl-butyl}-4-diethylamino-benzamide;
4-Diethylamino-N-{3-methyl-1-[6-oxo-4-(piperidine-2-carbonyl)-hexahydro-pyrrolo[3,2-b]pyrrole-1-carbonyl]-butyl}-benzamide;
N-{1-[4-(1-Acetyl-piperidine-2-carbonyl)-6-oxo-hexahydro-pyrrolo[3,2-b]pyrrole-1-carbonyl]-3-methyl-butyl}-4-diethylamino-benzamide;
4-Diethylamino-N-{3-methyl-1-[6-oxo-4-(tetrahydro-furan-2-carbonyl)-hexahydro-pyrrolo[3,2-b]pyrrole-1-carbonyl]-butyl}-benzamide;
N-{1-[4-(1-Amino-cyclobutanecarbonyl)-6-oxo-hexahydro-pyrrolo[3,2-b]pyrrole-1-carbonyl]-3-methyl-butyl}-4-diethylamino-benzamide;
N-{1-[4-(1-Amino-cyclopentanecarbonyl)-6-oxo-hexahydro-pyrrolo[3,2-b]pyrrole-1-carbonyl]-3-methyl-butyl}-4-diethylamino-benzamide;
N-{1-[4-(1-Amino-cyclohexanecarbonyl)-6-oxo-hexahydro-pyrrolo[3,2-b]pyrrole-1-carbonyl]-3-methyl-butyl}-4-diethylamino-benzamide;
N-{1-[4-(1-Acetylamino-cyclobutanecarbonyl)-6-oxo-hexahydro-pyrrolo[3,2-b]pyrrole-1-carbonyl]-3-methyl-butyl}-4-diethylamino-benzamide;
N-{1-[4-(1-Acetylamino-cyclopentanecarbonyl)-6-oxo-hexahydro-pyrrolo[3,2-b]pyrrole-1-carbonyl]-3-methyl-butyl}-4-diethylamino-benzamide;
N-{1-[4-(1-Acetylamino-cyclohexanecarbonyl)-6-oxo-hexahydro-pyrrolo[3,2-b]pyrrole-1-carbonyl]-3-methyl-butyl}-4-diethylamino-benzamide;
4-Diethylamino-N-{1-[4-(1-hydroxy-cyclobutanecarbonyl)-6-oxo-hexahydro-pyrrolo[3,2-b]pyrrole-1-carbonyl]-3-methyl-butyl}-benzamide;
4-Diethylamino-N-{1-[4-(1-hydroxy-cyclopentanecarbonyl)-6-oxo-hexahydro-pyrrolo[3,2-b]pyrrole-1-carbonyl]-3-methyl-butyl}-benzamide;
4-Diethylamino-N-{1-[4-(1-hydroxy-cyclohexanecarbonyl)-6-oxo-hexahydro-pyrrolo[3,2-b]pyrrole-1-carbonyl]-3-methyl-butyl}-benzamide;
4-Diethylamino-N-{1-[4-(1-methoxy-cyclobutanecarbonyl)-6-oxo-hexahydro-pyrrolo[3,2-b]pyrrole-1-carbonyl]-3-methyl-butyl}-benzamide;
4-Diethylamino-N-{1-[4-(1-methoxy-cyclopentanecarbonyl)-6-oxo-hexahydro-pyrrolo[3,2-b]pyrrole-1-carbonyl]-3-methyl-butyl}-benzamide;
4-Diethylamino-N-{1-[4-(1-methoxy-cyclohexanecarbonyl)-6-oxo-hexahydro-pyrrolo[3,2-b]pyrrole-1-carbonyl]-3-methyl-butyl}-benzamide;
N-{1-[4-(2-Amino-2-cyclopentyl-acetyl)-6-oxo-hexahydro-pyrrolo[3,2-b]pyrrole-1-carbonyl]-3-methyl-butyl}-4-diethylamino-benzamide;
N-{1-[4-(2-Amino-2-cyclohexyl-acetyl)-6-oxo-hexahydro-pyrrolo[3,2-b]pyrrole-1-carbonyl]-3-methyl-butyl}-4-diethylamino-benzamide;
N-{1-[4-(2-Acetylamino-2-cyclopentyl-acetyl)-6-oxo-hexahydro-pyrrolo[3,2-b]pyrrole-1-carbonyl]-3-methyl-butyl}-4-diethylamino-benzamide;
N-{1-[4-(2-Acetylamino-2-cyclohexyl-acetyl)-6-oxo-hexahydro-pyrrolo[3,2-b]pyrrole-1-carbonyl]-3-methyl-butyl}-4-diethylamino-benzamide;
N-{1-[4-(2-Acetylamino-propionyl)-6-oxo-hexahydro-pyrrolo[3,2-b]pyrrole-1-carbonyl]-3-methyl-butyl}-4-diethylamino-benzamide;
N-{1-[4-(2-Acetylamino-acetyl)-6-oxo-hexahydro-pyrrolo[3,2-b]pyrrole-1-carbonyl]-3-methyl-butyl}-4-diethylamino-benzamide;
N-(1-{4-[2-(Acetyl-methyl-amino)-acetyl]-6-oxo-hexahydro-pyrrolo[3,2-b]pyrrole-1-carbonyl}-3-methyl-butyl)-4-diethylamino-benzamide;
4-Diethylamino-N-{1-[4-(2-dimethylamino-acetyl)-6-oxo-hexahydro-pyrrolo[3,2-b]pyrrole-1-carbonyl]-3-methyl-butyl}-benzamide;
N-{1-[4-(2-Amino-butyryl)-6-oxo-hexahydro-pyrrolo[3,2-b]pyrrole-1-carbonyl]-3-methyl-butyl}-4-diethylamino-benzamide;
N-{1-[4-(2-Acetylamino-butyryl)-6-oxo-hexahydro-pyrrolo[3,2-b]pyrrole-1-carbonyl]-3-methyl-butyl}-4-diethylamino-benzamide;
N-{1-[4-(2-amino-3-methyl-butyryl)-6-oxo-hexahydro-pyrrolo[3,2-b]pyrrole-1-carbonyl]-3-methyl-butyl}-4-diethylamino-benzamide;
N-{1-[4-(2-Acetylamino-3-methyl-butyryl)-6-oxo-hexahydro-pyrrolo[3,2-b]pyrrole-1-carbonyl]-3-methyl-butyl}-4-diethylamino-benzamide;
N-{1-[4-(2-amino-3,3-dimethyl-butyryl)-6-oxo-hexahydro-pyrrolo[3,2-b]pyrrole-1-carbonyl]-3-methyl-butyl}-4-diethylamino-benzamide;
N-{1-[4-(2-Acetylamino-3,3-dimethyl-butyryl)-6-oxo-hexahydro-pyrrolo[3,2-b]pyrrole-1-carbonyl]-3-methyl-butyl}-4-diethylamino-benzamide;
N-{1-[4-(2-Amino-3-methyl-pentanoyl)-6-oxo-hexahydro-pyrrolo[3,2-b]pyrrole-1-carbonyl]-3-methyl-butyl}-4-diethylamino-benzamide;
N-{1-[4-(2-Acetylamino-3-methyl-pentanoyl)-6-oxo-hexahydro-pyrrolo[3,2-b]pyrrole-1-carbon yl]-3-methyl-butyl}-4-diethylamino-benzamide;
4-Diethylamino-N-[3-methyl-1-(6-oxo-4-pentanoyl-hexahydro-pyrrolo[3,2-b]pyrrole-1-carbonyl)-butyl]-benzamide;
4-Diethylamino-N-{3-methyl-1-[4-(3-methyl-pentanoyl)-6-oxo-hexahydro-pyrrolo[3,2-b]pyrrole-1-carbonyl]-butyl}-benzamide;
4-Diethylamino-N-{3-methyl-1-[4-(4-methyl-pentanoyl)-6-oxo-hexahydro-pyrrolo[3,2-b]pyrrole-1-carbonyl]-butyl}-benzamide;
N-{1-[4-(2-Amino-4-methyl-pentanoyl)-6-oxo-hexahydro-pyrrolo[3,2-b]pyrrole-1-carbonyl]-3-methyl-butyl}-4-diethylamino-benzamide;
N-{1-[4-(2-Acetylamino-4-methyl-pentanoyl)-6-oxo-hexahydro-pyrrolo[3,2-b]pyrrole-1-carbonyl]-3-methyl-butyl}-4-diethylamino-benzamide;
N-{1-[4-(2-Amino-4,4-dimethyl-pentanoyl)-6-oxo-hexahydro-pyrrolo[3,2-b]pyrrole-1-carbonyl]-3-methyl-butyl}-4-diethylamino-benzamide;
N-{1-[4-(2-Acetylamino-4,4-dimethyl-pentanoyl)-6-oxo-hexahydro-pyrrolo[3,2-b]pyrrole-1-carbonyl]-3-methyl-butyl}-4-diethylamino-benzamide;
N-{1-[4-(2-Amino-pentanoyl)-6-oxo-hexahydro-pyrrolo[3,2-b]pyrrole-1-carbonyl]-3-methyl-butyl}-4-diethylamino-benzamide;
N-{1-[4-(2-Acetylamino-pentanoyl)-6-oxo-hexahydro-pyrrolo[3,2-b]pyrrole-1-carbonyl]-3-methyl-butyl}-4-diethylamino-benzamide;
N-{1-[4-(2-Amino-5-methyl-hexanoyl)-6-oxo-hexahydro-pyrrolo[3,2-b]pyrrole-1-carbonyl]-3-methyl-butyl}-4-diethylamino-benzamide;
N-{1-[4-(2-Acetylamino-5-methyl-hexanoyl)-6-oxo-hexahydro-pyrrolo[3,2-b]pyrrole-1-carbonyl]-3-methyl-butyl}-4-diethylamino-benzamide;
N-{1-[4-(2-hydroxy-3-methyl-butyryl)-6-oxo-hexahydro-pyrrolo[3,2-b]pyrrole-1-carbonyl]-3-methyl-butyl}-4-diethylamino-benzamide;
N-{1-[4-(2-methoxy-3-methyl-butyryl)-6-oxo-hexahydro-pyrrolo[3,2-b]pyrrole-1-carbonyl]-3-methyl-butyl}-4-diethylamino-benzamide;
4-Diethylamino-N-{1-[4-(2-hydroxy-3,3-dimethyl-butyryl)-6-oxo-hexahydro-pyrrolo[3,2-b]pyrrole-1-carbonyl]-3-methyl-butyl}-benzamide;
4-Diethylamino-N-{1-[4-(2-methoxy-3,3-dimethyl-butyryl)-6-oxo-hexahydro-pyrrolo[3,2-b]pyrrole-1-carbonyl]-3-methyl-butyl}-benzamide;
4-Diethylamino-N-{1-[4-(2-hydroxy-3-methyl-pentanoyl)-6-oxo-hexahydro-pyrrolo[3,2-b]pyrrole-1-carbonyl]-3-methyl-butyl}-benzamide;
4-Diethylamino-N-{1-[4-(2-methoxy-3-methyl-pentanoyl)-6-oxo-hexahydro-pyrrolo[3,2-b]pyrrole-1-carbonyl]-3-methyl-butyl}-benzamide;
4-Diethylamino-N-{1-[4-(2-hydroxy-4-methyl-pentanoyl)-6-oxo-hexahydro-pyrrolo[3,2-b]pyrrole-1-carbonyl]-3-methyl-butyl}-benzamide;
4-Diethylamino-N-{1-[4-(2-methoxy-4-methyl-pentanoyl)-6-oxo-hexahydro-pyrrolo[3,2-b]pyrrole-1-carbonyl]-3-methyl-butyl}-benzamide;
4-Diethylamino-N-{1-[4-(2-hydroxy-4,4-dimethyl-pentanoyl)-6-oxo-hexahydro-pyrrolo[3,2-b]pyrrole-1-carbonyl]-3-methyl-butyl}-benzamide;
4-Diethylamino-N-{1-[4-(2-methoxy-4,4-dimethyl-pentanoyl)-6-oxo-hexahydro-pyrrolo[3,2-b]pyrrole-1-carbonyl]-3-methyl-butyl}-benzamide;
4-Diethylamino-N-{1-[4-(2-hydroxy-pentanoyl)-6-oxo-hexahydro-pyrrolo[3,2-b]pyrrole-1-carbonyl]-3-methyl-butyl}-benzamide;
4-Diethylamino-N-{1-[4-(2-methoxy-pentanoyl)-6-oxo-hexahydro-pyrrolo[3,2-b]pyrrole-1-carbonyl]-3-methyl-butyl}-benzamide;
4-Diethylamino-N-{1-[4-(2-hydroxy-5-methyl-hexanoyl)-6-oxo-hexahydro-pyrrolo[3,2-b]pyrrole-1-carbonyl]-3-methyl-butyl}-benzamide; and
4-Diethylamino-N-{1-[4-(2-methoxy-5-methyl-hexanoyl)-6-oxo-hexahydro-pyrrolo[3,2-b]pyrrole-1-carbonyl]-3-methyl-butyl}-benzamide.

52. A method of validating a known or putative cysteine protease inhibitor as a therapeutic target, the method comprising:
(a) assessing the in vitro binding of a compound as claimed in claim 1 to an isolated known or putative cysteine protease, providing a measure of potency; and optionally, one or more of the steps of:
(b) assessing the binding of the compound as claimed in claim 1 to closely related homologous proteases of the target and general housekeeping proteases to provide a measure of selectivity;
(c) monitoring a cell-based functional marker of a particular cysteine protease activity, in the presence of the compound; and
(d) monitoring an animal model-based functional marker of a particular cysteine protease activity in the presence of the compound.

53. (canceled)

54. (canceled)

55. (canceled)

56. (canceled)

57. (canceled)

58. A pharmaceutical or veterinary composition comprising one or more compounds as claimed in claim 1 and a pharmaceutically or veterinarily acceptable carrier.

59. (canceled)

60. A method of treating a patient suffering from a disease involving excessive bone loss the method comprising administering an effective amount of a compound as claimed in claim 1 to a patient in need of such treatment.

61. The method of claim 60, wherein the disease involving excessive bone loss is selected from the group consisting of osteoperosis, gingival disease, Paget's disease, hypercalcaemia of malignancy and metabolic bone disease.

62. The method of claim 61, wherein the gingival disease is selected from the group consisting of gingivitis and periodontitis.

63. A method of treating a patient suffering from disease involving matrix or cartilage degradation the method comprising administering an effective amount of a compound as claimed in claim 1 to a patient in need of such treatment.

64. The method of claim 63, wherein the disease is selected from the group consisting of osteoarthritis and rheumatoid arthritis.

65. A method of treating a patient suffering from neoplastic disease, the method comprising administering an effective amount of a compound as claimed in claim 1 to a patient in need of such treatment.

66. A method of treating a patient suffering from a disease or infection where an elevated level of cysteine protease is implicated the method comprising administering an effective amount of a compound as claimed in claim 1 to a patient in need of such treatment.

67. The method of claim 66, wherein the source of infection is selected from the group consisting of Pneumocystis carinii, Trypsanoma cruzi, Trypsanoma brucei brucei. Leishmania mexicana, Clostridium histolyticum, Staphylococcus aureua, foot and mouth disease virus, and Crithidia fusiculata.

68. The method of claim 66, wherein the disease is selected from the group consisting of an autoimmune disease, schistosomiasis, malaria, tumor metastasis, metachromatic leukodystrophy, mucular dystrophy, and amytrophy.

69. The method of claim 60, wherein the patient is a human.

70. The method of claim 63, wherin the patient is a human.

71. The method of claim 65, wherein the patient is a human.

72. The method of claim 67, wherein the patient is a human.

73. The compound of claim 13, wherein each of R¹⁶ and R¹⁷ is independently selected from the group consisting of hydrogen, a straight or branched alkyl chain, a straight or branched heteroalkyl chain, an optionally substituted arylalkyl chain or an optionally substituted arylheteroalkyl chain.

74. The compound of claim 26, wherein the halogen substituent is fluorine.
